(19) United States
(12) Patent Application Publication
(10) Pub. No.: US 2002/0061569 A1
(43) Pub. Date: May 23, 2002

(54) Identification of essential genes in prokaryotes

(76) Inventors: Robert Haselbeck, San Diego, CA (US); Kari L. Ohlsen, San Diego, CA (US); Judith W. Zyskind, La Jolla, CA (US); Daniel Wall, San Diego, CA (US); John D. Trawick, La Mesa, CA (US); Grant J. Carr, Escondido, CA (US); Robert T. Yamamoto, San Diego, CA (US); H. Howard Xu, San Diego, CA (US)

Correspondence Address: KNOBBE MARTENS OLSON & BEAR LLP, 620 NEWPORT CENTER DRIVE, SIXTEENTH FLOOR, NEWPORT BEACH, CA, 92660, US

(21) Appl. No.: 09/815,242

(22) Filed: Mar. 21, 2001

Related U.S. Application Data
(60) Provisional application No. 60/191,078, filed on Mar. 21, 2000.
(60) Provisional application No. 60/206,848, filed on May 23, 2000.
(60) Provisional application No. 60/207,727, filed on May 26, 2000.
(60) Provisional application No. 60/242,578, filed on Oct. 23, 2000.
(60) Provisional application No. 60/253,625, filed on Nov. 27, 2000.
(60) Provisional application No. 60/257,931, filed on Dec. 22, 2000.
(60) Provisional application No. 60/269,308, filed on Feb. 16, 2001.

(51) Int. Cl. C12N 9/00; C12Q 1/68; C07H 21/04; C07K 14/435; C12P 21/02; C12N 5/06

(52) U.S. Cl. 435/183; 530/350; 435/6; 536/23.2; 435/69.1; 435/320.1; 435/325

(57) ABSTRACT

The sequences of antisense nucleic acids which inhibit the proliferation of prokaryotes are disclosed. Cell-based assays which employ the antisense nucleic acids to identify and develop antibiotics are also disclosed. The antisense nucleic acids can also be used to identify proteins required for proliferation, express these proteins or portions thereof, obtain antibodies capable of specifically binding to the expressed proteins, and to use those expressed proteins as a screen to isolate candidate molecules for rational drug discovery programs. The nucleic acids can also be used to screen for homologous nucleic acids that are required for proliferation in cells other than Staphylococcus aureus, Salmonella typhimurium, Klebsiella pneumoniae, and Pseudomonas aeruginosa. The nucleic acids of the present invention can also be used in various assay systems to screen for proliferation required genes in other organisms.

### RELATED APPLICATIONS

This application claims priority from U.S. Provisional Patent Application Ser. No. 60/191,078, filed Mar. 21, 2000, U.S. Provisional Patent Application Ser. No. 60/206,848, filed May 23, 2000, U.S. Provisional Patent Application Ser. No. 60/207,727, filed May 26, 2000, U.S. Provisional Patent Application Ser. No. 60/242,578, filed Oct. 23, 2000, U.S. Provisional Patent Application Ser. No. 60/253,625, filed Nov. 27, 2000, U.S. Provisional Patent Application Ser. No. 60/257,931, filed Dec. 22, 2000, and U.S. Provisional Patent Application Ser. No. 60/269,308, filed Feb. 16, 2001 the disclosures of which are incorporated herein by reference in their entireties.

### SEQUENCE LISTING

The present application is being filed along with duplicate copies of a CD-ROM marked “Copy 1” and “Copy 2” containing a Sequence Listing in electronic format. The duplicate copies of the CD-ROM each contain a file entitled SEQLIST_FINAL_{—}9PM created on Mar. 20, 2001 which is 37,487,912 bytes in size. The information on these duplicate CD-ROMs is incorporated herein by reference in its entirety.

### BACKGROUND OF THE INVENTION

Since the discovery of penicillin, the use of antibiotics to treat the ravages of bacterial infections has saved millions of lives. With the advent of these “miracle drugs,” for a time it was popularly believed that humanity might, once and for all, be saved from the scourge of bacterial infections. In fact, during the 1980s and early 1990s, many large pharmaceutical companies cut back or eliminated antibiotics research and development. They believed that infectious disease caused by bacteria finally had been conquered and that markets for new drugs were limited. Unfortunately, this belief was overly optimistic.

The tide is beginning to turn in favor of the bacteria as reports of drug resistant bacteria become more frequent. The United States Centers for Disease Control announced that one of the most powerful known antibiotics, vancomycin, was unable to treat an infection of the common Staphylococcus aureus (staph). This organism is commonly found in our environment and is responsible for many nosocomial infections. The import of this announcement becomes clear when one considers that vancomycin was used for years to treat infections caused by Staphylococcus species as well as other stubborn strains of bacteria. In short, bacteria are becoming resistant to our most powerful antibiotics. If this trend continues, it is conceivable that we will return to a time when what are presently considered minor bacterial infections are fatal diseases.

Over-prescription and improper prescription habits by some physicians have caused an indiscriminate increase in the availability of antibiotics to the public. The patients are also partly responsible, since they will often improperly use the drug, thereby generating yet another population of bacteria that is resistant, in whole or in part, to traditional antibiotics.

The bacterial pathogens that have haunted humanity remain, in spite of the development of modern scientific practices to deal with the diseases that they cause. Drug resistant bacteria are now an increasing threat to the health of humanity. A new generation of antibiotics is needed to once again deal with the pending health threat that bacteria present.

### Discovery of New Antibiotics

As more and more bacterial strains become resistant to the panel of available antibiotics, new antibiotics are required to treat infections. In the past, practitioners of pharmacology would have to rely upon traditional methods of drug discovery to generate novel, safe and efficacious compounds for the treatment of disease. Traditional drug discovery methods involve blindly testing potential drug candidate-molecules, often selected at random, in the hope that one might prove to be an effective treatment for some disease. The process is painstaking and laborious, with no guarantee of success. Today, the average cost to discover and develop a new drug exceeds US $500 million, and the average time from laboratory to patient is 15 years. Improving this process, even incrementally, would represent a huge advance in the generation of novel antimicrobial agents.

Newly emerging practices in drug discovery utilize a number of biochemical techniques to provide for directed approaches to creating new drugs, rather than discovering them at random. For example, gene sequences and proteins encoded thereby that are required for the proliferation of a cell or microorganism make excellent targets since exposure of bacteria to compounds active against these targets would result in the inactivation of the cell or microorganism. Once a target is identified, biochemical analysis of that target can be used to discover or to design molecules that interact with and alter the functions of the target. Use of physical and computational techniques to analyze structural and biochemical properties of targets in order to derive compounds that interact with such targets is called rational drug design and offers great potential. Thus, emerging drug discovery practices use molecular modeling techniques, combinatorial chemistry approaches, and other means to produce and screen and/or design large numbers of candidate compounds.

Nevertheless, while this approach to drug discovery is clearly the way of the future, problems remain. For example, the initial step of identifying molecular targets for investigation can be an extremely time consuming task. It may also be difficult to design molecules that interact with the target by using computer modeling techniques. Furthermore, in cases where the function of the target is not known or is poorly understood, it may be difficult to design assays to detect molecules that interact with and alter the functions of the target. To improve the rate of novel drug discovery and development, methods of identifying important molecular targets in pathogenic cells or microorganisms and methods for identifying molecules that interact with and alter the functions of such molecular targets are urgently required.

Staphylococcus aureus is a Gram positive microorganism which is the causative agent of many infectious diseases. Local infection by Staphylococcus aureus can cause abscesses on skin and cellulitis in subcutaneous tissues and can lead to toxin-related diseases such as toxic shock and scalded skin syndromes. Staphylococcus aureus can cause serious systemic infections such as osteomyelitis, endocarditis, pneumonia, and septicemia. Staphylococcus aureus is also a common cause of food poisoning, often arising from contact between prepared food and infected food industry workers. Antibiotic resistant strains of Staphylococcus aureus have recently been identified, including those that are now resistant to all available antibiotics, thereby severely limiting the options of care available to physicians.

Pseudomonas aerginosa is an important Gram-negative opportunistic pathogen. It is the most common Gram-negative found in nosocomial infections. P. aeruginosa is responsible for 16% of nosocomial pneumonia cases, 12% of hospital-acquired urinary tract infections, 8% of surgical wound infections, and 10% of bloodstream infections. Immunocompromised patients, such as neutropenic cancer and bone marrow transplant patients, are particular susceptible to opportunistic infections. In this group of patients, P. aeruginosa is responsible for pneumonia and septicemia with attributable deaths reaching 30%. P. aeruginosa is also one of the most common and lethal pathogens responsible for ventilator-associated pneumonia in intubated patients, with directly attributable death rates reaching 38%. Although P. aeruginosa outbreaks in bum patients are rare, it is associated with 60% death rates. In the AIDS population, P. aerginosa is associated with 50% of deaths. Cystic fibrosis patients are characteristically susceptible to chronic infection by P. aeruginosa, which is responsible for high rates of illness and death. Current antibiotics work poorly for CF infections (Van Delden & Igelwski. 1998. Emerging Infectious Diseases 4:551-560; references therein).

The gram-negative enteric bacterial genus, Salmonella, encompasses at least 2 species. One of these, S. enterica, is divided into multiple subspecies and thousands of serotypes or serovars (Brenner, et al. 2000 J. Clin. Microbiol. 38:2465-2467). The S. enterica human pathogens include serovars Typhi, Paratyphi, Typhimurium, Cholerasuis, and many others deemed so closely related that they are variants of a widespread species. Worldwide, disease in humans caused by Salmonella is a very serious problem. In many developing countries, S. enterica ser. Typhi still causes often-fatal typhoid fever. This problem has been reduced or eliminated in wealthy industrial states. However, enteritis induced by Salmonella is widespread and is the second most common disease caused by contaminated food in the United States (Edwards, B H 1999 “Salmonella and Shigella species” Clin. Lab Med. 19(3):469-487). Though usually self-limiting in healthy individuals, others such as children, seniors, and those with compromising illnesses can be at much greater risk of serious illness and death.

Some S. enterica serovars (e.g. Typhimurium) cause a localized infection in the gastrointestinal tract. Other serovars (i.e. Typhi and Paratyphi) cause a much more serious systemic infection. In animal models, these roles can be reversed which has allowed the use of the relatively safe S. enterica ser. Typhimurium as a surrogate in mice for the typhoid fever agent, S. enterica ser. Typhi. In mice, S. enterica ser Typhimurium causes a systemic infection similar in outcome to typhoid fever. Years of study of the Salmonella have led to the identification of many determinants of virulence in animals and humans. Salmonella is interesting in its ability to localize to and invade the intestinal epithelium, induce morphologic changes in target cells via injection of certain cell-remodeling proteins, and to reside intracellularly in membrane-bound vesicles (Wallis, T S and Galyov, EE 2000 “Molecular basis of Salmonella-induced enteritis.” Molec. Microb. 36:997-1005; Falkow, S “The evolution of pathogenicity in Escherichia, Shigella, and Salmonella,” Chap. 149 in Neidhardt, et al. eds pp 2723-2729; Gulig, P A “Pathogenesis of Systemic Disease,” Chap. 152 in Neidhardt, et al. ppp 2774-2787). The immediate infection often results in a severe watery diarrhea but Salmonella also can establish and maintain a subclinical carrier state in some individuals. Spread is via food contaminated with sewage.

The gene products implicated in Salmonella pathogenesis include type three secretion systems (TTSS), proteins affecting cytoplasmic structure of the target cells, many proteins carrying out functions necessary for survival and proliferation of Salmonella in the host, as well as “traditional” factors such as endotoxin and secreted exotoxins. Additionally, there must be factors mediating species-specific illnesses. Despite this most of the genomes of S. enterica ser. Typhi (see http://www.sanger.ac.uk/Proiects/S_typhi/ for the genome database) and S. enterica ser. Typhimurium (see http://genome.wustl.edu/gsc/bacterial/salmonella.shtml for the genome database) are highly conserved and are mutually useful for gene identification in multiple serovars. The Salmonella are a complex group of enteric bacteria causing disease similar to but distinct from other gram-negative enterics such as E. coli and have been a focus of biomedical research for the last century.

Enterococcus faecalis, a Gram-positive bacterium, is by far the most common member of the enterococci to cause infections in humans. Enterococcus faecium generally accounts for less than 20% of clinical isolates. Enterococci infections are mostly hospital-acquired though they are also associated with some community-acquired infections. Of nosocomial infections enterococci account for 12% of bacteremia, 15% of surgical wound infections, 14% of urinary tract infections, and 5 to 15% of endocarditis cases (Huycke, M. M., D. F., Sahm and M. S. Gilmore. 1998. Emerging Infectious Diseases 4:239-249). Additionally enterococci are frequently associated with intraabdominal and pelvic infections. Enterococci infections are often hard to treat because they are resistant to a vast array of antimicrobial drugs, including aminoglycosides, penicillin, ampicillin and vancomycin. The development of multiple-drug resistant (MDR) enterococci has made this bacteria a major concern for treating nosocomial infections.

These reasons underscore the urgency of developing new antibiotics that are effective against Staphylococcus aureus, Salmonella typhimurium, Klebsiella pneumoniae, Pseudomonas aeruginosa, and Enterococcus faecalis. Accordingly, there is an urgent need for more novel methods to identify and characterize bacterial genomic sequences that encode gene products involved in proliferation, and are thereby potential new targets for antibiotic development. Prior to the present invention, the discovery of Staphylococcus aureus, Salmonella typhimurium, Klebsiella pneumoniae, and Pseudomonas aerginosa and Enterococcus faecalis genes required for proliferation of the microorganism was a painstaking and slow process. While the detection of new cellular drug targets within a Staphylococcus aureus, Salmonella typhimurium, Klebsiella pneumoniae, Pseudomonas aeruginosa or Enterococcus faecalis cell is key for novel antibiotic development, the current methods of drug target discovery available prior to this invention have required painstaking processes requiring years of effort.

### SUMMARY OF THE INVENTION

Some aspects of the present invention are described in the numbered paragraphs below.

1. A purified or isolated nucleic acid sequence comprising a nucleotide sequence consisting essentially of one of SEQ ID NOs: 8-3795, wherein expression of said nucleic acid inhibits proliferation of a cell.

2. The nucleic acid sequence of Paragraph 1, wherein said nucleotide sequence is complementary to at least a portion of a coding sequence of a gene whose expression is required for proliferation of a cell.

3. The nucleic acid of Paragraph 1, wherein said nucleic acid sequence is complementary to at least a portion of a nucleotide sequence of an RNA required for proliferation of a cell.

4. The nucleic acid of Paragraph 3, wherein said RNA is an RNA comprising a sequence of nucleotides encoding more than one gene product.

5. A purified or isolated nucleic acid comprising a fragment of one of SEQ ID NOs.: 8-3795, said fragment selected from the group consisting of fragments comprising at least 10, at least 20, at least 25, at least 30, at least 50 and more than 50 consecutive nucleotides of one of SEQ ID NOs: 8-3795.

6. The fragment of Paragraph 5, wherein said fragment is included in a nucleic acid obtained from an organism selected from the group consisting of Anaplasma marginale, Aspergillus fumigatus, Bacillus anthracis, Bacterioides fragilis Bordetella pertussis, Burkholderia cepacia, Campylobacter jejuni, Candida albicans, Candida glabrata (also called Torulopsis glabrata), Candida tropicalis, Candida parapsilosis, Candida guilliermondii, Candida krusei, Candida kefyr (also called Candida pseudotropicalis), Candida dubliniensis, Chlamydia pneumoniae, Chlamydia trachomatus, Clostridium botulinum, Clostridium difficile, Clostridium perfringens, Coccidiodes immitis, Corynebacterium diptheriae, Cryptococcus neoformans, Enterobacter cloacae, Enterococcus faecalis, Enterococcus faecium, Escherichia coli, Haemophilus influenzae, Helicobacter pylori, Histoplasma capsulatum, Klebsiella pneumoniae, Listeria monocytogenes, Mycobacterium leprae, Mycobacterium tuberculosis, Neisseria gonorrhoeae, Neisseria meningitidis, Nocardia asteroides, Pasteurella haemolytica, Pasteurella multocida, Pneumocystis carinii, Proteus vulgaris, Pseudomonas aeruginosa, Salmonella bongori, Salmonella cholerasuis, Salmonella enterica, Salmonella paratyphi, Salmonella typhi, Salmonella typhimurium, Staphylococcus aureus, Listeria monocytogenes, Moxarella catarrhalis, Shigella boydii, Shigella dysenteriae, Shigella flexneri, Shigella sonnei, Staphylococcus epidermidis, Streptococcus pneumoniae, Streptococcus mutans, Treponema pallidum, Yersinia enterocolitica, Yersinia pestis and any species falling within the genera of any of the above species.

7. The fragment of Paragraph 5, wherein said fragment is included in a nucleic acid obtained from an organism other than Escherichia coli.

8. A vector comprising a promoter operably linked to the nucleic acid of any one of Paragraphs 1-7.

9. The vector of Paragraph 8, wherein said promoter is active in a microorganism selected from the group consisting of Anaplasma marginale, Aspergillus fumigatus, Bacillus anthracis, Bacterioides fragilis Bordetella pertussis, Burkholderia cepacia, Campylobacter jejuni, Candida albicans, Candida glabrata (also called Torulopsis glabrata), Candida tropicalis, Candida parapsilosis, Candida guilliermondii, Candida krusei, Candida kefyr (also called Candida pseudotropicalis), Candida dubliniensis, Chlamydia pneumoniae, Chlamydia trachomatus, Clostridium botulinum, Clostridium difficile, Clostridium perfringens, Coccidiodes immitis, Corynebacterium diptheriae, Cryptococcus neoformans, Enterobacter cloacae, Enterococcus faecalis, Enterococcus faecium, Escherichia coli, Haemophilus influenzae, Helicobacter pylori, Histoplasma capsulatum, Klebsiella pneumoniae, Listeria monocytogenes, Mycobacterium leprae, Mycobacterium tuberculosis, Neisseria gonorrhoeae, Neisseria meningitidis, Nocardia asteroides, Pasteurella haemolytica, Pasteurella multocida, Pneumocystis carinii, Proteus vulgaris, Pseudomonas aeruginosa, Salmonella bongori, Salmonella cholerasuis, Salmonella enterica, Salmonella paratyphi, Salmonella typhi, Salmonella typhimurium, Staphylococcus aureus, Listeria monocytogenes, Moxarella catarrhalis, Shigella boydii, Shigella dysenteriae, Shigella flexneri, Shigella sonnei, Staphylococcus epidermidis, Streptococcus pneumoniae, Streptococcus mutans, Treponema pallidum, Yersinia enterocolitica, Yersinia pestis and any species falling within the genera of any of the above species.

10. A host cell containing the vector of Paragraph 8 or Paragraph 9.

11. A purified or isolated antisense nucleic acid comprising a nucleotide sequence complementary to at least a portion of an intragenic sequence, intergenic sequence, sequences spanning at least a portion of two or more genes, 5′ noncoding region, or 3′ noncoding region within an operon comprising a proliferation-required gene whose activity or expression is inhibited by an antisense nucleic acid comprising the nucleotide sequence of one of SEQ ID NOs.: 8-3795.

12. The purified or isolated antisense nucleic acid of Paragraph 11, wherein said antisense nucleic acid is complementary to a nucleic acid from an organism selected from the group consisting of Anaplasma marginale, Aspergillus fumigatus, Bacillus anthracis, Bacterioides fragilis Bordetella pertussis, Burkholderia cepacia, Campylobacter jejuni, Candida albicans, Candida glabrata (also called Torulopsis glabrata), Candida tropicalis, Candida parapsilosis, Candida guilliermondii, Candida krusei, Candida kefyr (also called Candida pseudotropicalis), Candida dubliniensis, Chlamydia pneumoniae, Chlamydia trachomatus, Clostridium botulinum, Clostridium difficile, Clostridium perfringens, Coccidiodes immitis, Corynebacterium diptheriae, Cryptococcus neoformans, Enterobacter cloacae, Enterococcus faecalis, Enterococcus faecium, Escherichia coli, Haemophilus influenzae, Helicobacter pylori, Histoplasma capsulatum, Klebsiella pneumoniae, Listeria monocytogenes, Mycobacterium leprae, Mycobacterium tuberculosis, Neisseria gonorrhoeae, Neisseria meningitidis, Nocardia asteroides, Pasteurella haemolytica, Pasteurella multocida, Pneumocystis carinii, Proteus vulgaris, Pseudomonas aeruginosa, Salmonella bongori, Salmonella cholerasuis, Salmonella enterica, Salmonella paratyphi, Salmonella typhi, Salmonella typhimurium, Staphylococcus aureus, Listeria monocytogenes, Moxarella catarrhalis, Shigella boydii, Shigella dysenteriae, Shigella flexneri, Shigella sonnei, Staphylococcus epidermidis, Streptococcus pneumoniae, Streptococcus mutans, Treponema pallidum, Yersinia enterocolitica, Yersinia pestis and any species falling within the genera of any of the above species.

13. The purified or isolated antisense nucleic acid of Paragraph 11, wherein said nucleotide sequence is complementary to a nucleotide sequence of a nucleic acid from an organism other than E. coli.

14. The purified or isolated antisense nucleic acid of Paragraph 11, wherein said proliferation-required gene comprises a nucleotide sequence selected from the group consisting of SEQ ID NOS.: 3796-3800, 3806-4860, 5916-10012.

15. A purified or isolated nucleic acid comprising a nucleotide sequence having at least 70% identity to a nucleotide sequence selected from the group consisting of SEQ ID NOs.: 8-3795, fragments comprising at least 25 consecutive nucleotides of SEQ ID NOs.: 8-3795, the nucleotide sequences complementary to SEQ ID NOs.: 8-3795 and the sequences complementary to fragments comprising at least 25 consecutive nucleotides of SEQ ID NOs.: 8-3795 as determined using BLASTN version 2.0 with the default parameters.

16. The purified or isolated nucleic acid of Paragraph 15, wherein said nucleic acid is obtained from an organism selected from the group consisting of Anaplasma marginale, Aspergillus fumigatus, Bacillus anthracis, Bacterioides fragilis Bordetella pertussis, Burkholderia cepacia, Campylobacter jejuni, Candida albicans, Candida glabrata (also called Torulopsis glabrata), Candida tropicalis, Candida parapsilosis, Candida guilliermondii, Candida krusei, Candida kefyr (also called Candida pseudotropicalis), Candida dubliniensis, Chlamydia pneumoniae, Chlamydia trachomatus, Clostridium botulinum, Clostridium difficile, Clostridium perfringens, Coccidiodes immitis, Corynebacterium diptheriae, Cryptococcus neoformans, Enterobacter cloacae, Enterococcus faecalis, Enterococcus faecium, Escherichia coli, Haemophilus influenzae, Helicobacter pylori, Histoplasma capsulatum, Klebsiella pneumoniae, Listeria monocytogenes, Mycobacterium leprae, Mycobacterium tuberculosis, Neisseria gonorrhoeae, Neisseria meningitidis, Nocardia asteroides, Pasteurella haemolytica, Pasteurella multocida, Pneumocystis carinii, Proteus vulgaris, Pseudomonas aeruginosa, Salmonella bongori, Salmonella cholerasuis, Salmonella enterica, Salmonella paratyphi, Salmonella typhi, Salmonella typhimurium, Staphylococcus aureus, Listeria monocytogenes, Moxarella catarrhalis, Shigella boydii, Shigella dysenteriae, Shigella flexneri, Shigella sonnei, Staphylococcus epidermidis, Streptococcus pneumoniae, Streptococcus mutans, Treponema pallidum, Yersinia enterocolitica, Yersinia pestis and any species falling within the genera of any of the above species.

17. The nucleic acid of Paragraph 15, wherein said nucleic acid is obtained from an organism other than E. coli.

18. A vector comprising a promoter operably linked to a nucleic acid encoding a polypeptide whose expression is inhibited by an antisense nucleic acid comprising a nucleotide sequence of any one of SEQ ID NOs.: 8-3795.

19. The vector of Paragraph 18, wherein said nucleic acid encoding said polypeptide is obtained from an organism selected from the group consisting of Anaplasma marginale, Aspergillus fumigatus, Bacillus anthracis, Bacterioides fragilis Bordetella pertussis, Burkholderia cepacia, Campylobacter jejuni, Candida albicans, Candida glabrata (also called Torulopsis glabrata), Candida tropicalis, Candida parapsilosis, Candida guilliermondii, Candida krusei, Candida kefyr (also called Candida pseudotropicalis), Candida dubliniensis, Chlamydia pneumoniae, Chlamydia trachomatus, Clostridium botulinum, Clostridium difficile, Clostridium perfringens, Coccidiodes immitis, Corynebacterium diptheriae, Cryptococcus neoformans, Enterobacter cloacae, Enterococcus faecalis, Enterococcus faecium, Escherichia coli, Haemophilus influenzae, Helicobacter pylori, Histoplasma capsulatum, Klebsiella pneumoniae, Listeria monocytogenes, Mycobacterium leprae, Mycobacterium tuberculosis, Neisseria gonorrhoeae, Neisseria meningitidis, Nocardia asteroides, Pasteurella haemolytica, Pasteurella multocida, Pneumocystis carinii, Proteus vulgaris, Pseudomonas aeruginosa, Salmonella bongori, Salmonella cholerasuis, Salmonella enterica, Salmonella paratyphi, Salmonella typhi, Salmonella typhimurium, Staphylococcus aureus, Listeria monocytogenes, Moxarella catarrhalis, Shigella boydii, Shigella dysenteriae, Shigella flexneri, Shigella sonnei, Staphylococcus epidermidis, Streptococcus pneumoniae, Streptococcus mutans, Treponema pallidum, Yersinia enterocolitica, Yersinia pestis and any species falling within the genera of any of the above species.

20. The vector of Paragraph 18, wherein said nucleotide sequence encoding said polypeptide is obtained from an organism other than E. coli.

21. A host cell containing the vector of Paragraph 18.

22. The vector of Paragraph 18, wherein said polypeptide comprises a polypeptide comprising an amino acid sequence selected from the group consisting of SEQ ID NOs: 3801-3805, 4861-5915, 10013-14110.

23. The vector of Paragraph 18, wherein said promoter is operably linked to a nucleic acid comprising a nucleotide sequence selected from the group consisting of SEQ ID NOS.: 3796-3800, 3806-4860, 5916-10012.

24. A purified or isolated polypeptide comprising a polypeptide whose expression is inhibited by an antisense nucleic acid comprising a nucleotide sequence of any one of SEQ ID NOs.: 8-3795, or a fragment selected from the group consisting of fragments comprising at least 5, at least 10, at least 20, at least 30, at least 40, at least 50, at least 60 or more than 60 consecutive amino acids of one of the said polypeptides.

25. The polypeptide of Paragraph 24, wherein said polypeptide comprises an amino acid sequence of any one of SEQ ID NOs.: 3801-3805, 4861-5915, 10013-14110 or a fragment comprising at least 5, at least 10, at least 20, at least 30, at least 40, at least 50, at least 60 or more than 60 consecutive amino acids of a polypeptide comprising an amino acid sequence selected from the group consisting of SEQ ID NOs.: 3801-3805, 4861-5915, 10013-14110.

26. The polypeptide of Paragraph 24, wherein said polypeptide is obtained from an organism selected from the group consisting of Anaplasma marginale, Aspergillus fumigatus, Bacillus anthracis, Bacterioides fragilis Bordetella pertussis, Burkholderia cepacia, Campylobacter jejuni, Candida albicans, Candida glabrata (also called Torulopsis glabrata), Candida tropicalis, Candida parapsilosis, Candida guilliermondii, Candida krusei, Candida kefyr (also called Candida pseudotropicalis), Candida dubliniensis, Chlamydia pneumoniae, Chlamydia trachomatus, Clostridium botulinum, Clostridium difficile, Clostridium perfringens, Coccidiodes immitis, Corynebacterium diptheriae, Cryptococcus neoformans, Enterobacter cloacae, Enterococcus faecalis, Enterococcus faecium, Escherichia coli, Haemophilus influenzae, Helicobacter pylori, Histoplasma capsulatum, Klebsiella pneumoniae, Listeria monocytogenes, Mycobacterium leprae, Mycobacterium tuberculosis, Neisseria gonorrhoeae, Neisseria meningitidis, Nocardia asteroides, Pasteurella haemolytica, Pasteurella multocida, Pneumocystis carinii, Proteus vulgaris, Pseudomonas aeruginosa, Salmonella bongori, Salmonella cholerasuis, Salmonella enterica, Salmonella paratyphi, Salmonella typhi, Salmonella typhimurium, Staphylococcus aureus, Listeria monocytogenes, Moxarella catarrhalis, Shigella boydii, Shigella dysenteriae, Shigella flexneri, Shigella sonnei, Staphylococcus epidermidis, Streptococcus pneumoniae, Streptococcus mutans, Treponema pallidum, Yersinia enterocolitica, Yersinia pestis and any species falling within the genera of any of the above species.

27. The polypeptide of Paragraph 24, wherein said polypeptide is obtained from an organism other than E. coli.

28. A purified or isolated polypeptide comprising a polypeptide having at least 25% amino acid identity to a polypeptide whose expression is inhibited by a nucleic acid comprising a nucleotide sequence selected from the group consisting of SEQ ID NOs.: 8-3795, or at least 25% amino acid identity to a fragment comprising at least 10, at least 20, at least 30, at least 40, at least 50, at least 60 or more than 60 consecutive amino acids of a polypeptide whose expression is inhibited by a nucleic acid comprising a nucleotide sequence selected from the group consisting of SEQ ID NOs.: 8-3795 as determined using FASTA version 3.0t78 with the default parameters.

29. The polypeptide of Paragraph 28, wherein said polypeptide has at least 25% identity to a polypeptide comprising one of SEQ ID NOs: 3801-3805, 4861-5915, 10013-14110 or at least 25% identity to a fragment comprising at least 5, at least 10, at least 20, at least 30, at least 40, at least 50, at least 60 or more than 60 consecutive amino acids of a polypeptide comprising one of SEQ ID NOs.: 3801-3805, 4861-5915, 10013-14110 as determined using FASTA version 3.0t78 with the default parameters.

30. The polypeptide of Paragraph 28, wherein said polypeptide is obtained from an organism selected from the group consisting of Anaplasma marginale, Aspergillus fumigatus, Bacillus anthracis, Bacterioides fragilis Bordetella pertussis, Burkholderia cepacia, Campylobacter jejuni, Candida albicans, Candida glabrata (also called Torulopsis glabrata), Candida tropicalis, Candida parapsilosis, Candida guilliermondii, Candida krusei, Candida kefyr (also called Candida pseudotropicalis), Candida dubliniensis, Chlamydia pneumoniae, Chlamydia trachomatus, Clostridium botulinum, Clostridium difficile, Clostridium perfringens, Coccidiodes immitis, Corynebacterium diptheriae, Cryptococcus neoformans, Enterobacter cloacae, Enterococcus faecalis, Enterococcus faecium, Escherichia coli, Haemophilus influenzae, Helicobacter pylori, Histoplasma capsulatum, Klebsiella pneumoniae, Listeria monocytogenes, Mycobacterium leprae, Mycobacterium tuberculosis, Neisseria gonorrhoeae, Neisseria meningitidis, Nocardia asteroides, Pasteurella haemolytica, Pasteurella multocida, Pneumocystis carinii, Proteus vulgaris, Pseudomonas aeruginosa, Salmonella bongori, Salmonella cholerasuis, Salmonella enterica, Salmonella paratyphi, Salmonella typhi, Salmonella typhimurium, Staphylococcus aureus, Listeria monocytogenes, Moxarella catarrhalis, Shigella boydii, Shigella dysenteriae, Shigella flexneri, Shigella sonnei, Staphylococcus epidermidis, Streptococcus pneumoniae, Streptococcus mutans, Treponema pallidum, Yersinia enterocolitica, Yersinia pestis and any species falling within the genera of any of the above species.

31. The polypeptide of Paragraph 28, wherein said polypeptide is obtained from an organism other than E. coli.

32. An antibody capable of specifically binding the polypeptide of one of Paragraphs 28-31.

33. A method of producing a polypeptide, comprising introducing a vector comprising a promoter operably linked to a nucleic acid comprising a nucleotide sequence encoding a polypeptide whose expression is inhibited by an antisense nucleic acid comprising one of SEQ ID NOs.: 8-3795 into a cell.

34. The method of Paragraph 33, further comprising the step of isolating said polypeptide.

35. The method of Paragraph 33, wherein said polypeptide comprises an amino acid sequence selected from the group consisting of SEQ ID NOs.: 3801-3805, 4861-5915, 10013-14110.

36. The method of Paragraph 33, wherein said nucleic acid encoding said polypeptide is obtained from an organism selected from the group consisting of Anaplasma marginale, Aspergillus fumigatus, Bacillus anthracis, Bacterioides fragilis Bordetella pertussis, Burkholderia cepacia, Campylobacter jejuni, Candida albicans, Candida glabrata (also called Torulopsis glabrata), Candida tropicalis, Candida parapsilosis, Candida guilliermondii, Candida krusei, Candida kefyr (also called Candida pseudotropicalis), Candida dubliniensis, Chlamydia pneumoniae, Chlamydia trachomatus, Clostridium botulinum, Clostridium difficile, Clostridium perfringens, Coccidiodes immitis, Corynebacterium diptheriae, Cryptococcus neoformans, Enterobacter cloacae, Enterococcus faecalis, Enterococcus faecium, Escherichia coli, Haemophilus influenzae, Helicobacter pylori, Histoplasma capsulatum, Klebsiella pneumoniae, Listeria monocytogenes, Mycobacterium leprae, Mycobacterium tuberculosis, Neisseria gonorrhoeae, Neisseria meningitidis, Nocardia asteroides, Pasteurella haemolytica, Pasteurella multocida, Pneumocystis carinii, Proteus vulgaris, Pseudomonas aeruginosa, Salmonella bongori, Salmonella cholerasuis, Salmonella enterica, Salmonella paratyphi, Salmonella typhi, Salmonella typhimurium, Staphylococcus aureus, Listeria monocytogenes, Moxarella catarrhalis, Shigella boydii, Shigella dysenteriae, Shigella flexneri, Shigella sonnei, Staphylococcus epidermidis, Streptococcus pneumoniae, Streptococcus mutans, Treponema pallidum, Yersinia enterocolitica, Yersinia pestis and any species falling within the genera of any of the above species.

37. The method of Paragraph 33, wherein said nucleic acid encoding said polypeptide is obtained from an organism other than E. coli.

38. The method of Paragraph 33, wherein said promoter is operably linked to a nucleic acid comprising a nucleotide sequence selected from the group consisting of SEQ ID NOS.: 3796-3800, 3806-4860, 5916-10012.

39. A method of inhibiting proliferation of a cell in an individual comprising inhibiting the activity or reducing the amount of a gene product whose expression is inhibited by an antisense nucleic acid comprising a nucleotide sequence selected from the group consisting of SEQ ID NOs.: 8-3795 or inhibiting the activity or reducing the amount of a nucleic acid encoding said gene product.

40. The method of Paragraph 39, wherein said method comprises inhibiting said activity or reducing said amount of a gene product in an organism selected from the group consisting of Anaplasma marginale, Aspergillus fumigatus, Bacillus anthracis, Bacterioides fragilis Bordetella pertussis, Burkholderia cepacia, Campylobacter jejuni, Candida albicans, Candida glabrata (also called Torulopsis glabrata), Candida tropicalis, Candida parapsilosis, Candida guilliermondii, Candida krusei, Candida kefyr (also called Candida pseudotropicalis), Candida dubliniensis, Chlamydia pneumoniae, Chlamydia trachomatus, Clostridium botulinum, Clostridium difficile, Clostridium perfringens, Coccidiodes immitis, Corynebacterium diptheriae, Cryptococcus neoformans, Enterobacter cloacae, Enterococcus faecalis, Enterococcus faecium, Escherichia coli, Haemophilus influenzae, Helicobacter pylori, Histoplasma capsulatum, Klebsiella pneumoniae, Listeria monocytogenes, Mycobacterium leprae, Mycobacterium tuberculosis, Neisseria gonorrhoeae, Neisseria meningitidis, Nocardia asteroides, Pasteurella haemolytica, Pasteurella multocida, Pneumocystis carinii, Proteus vulgaris, Pseudomonas aeruginosa, Salmonella bongori, Salmonella cholerasuis, Salmonella enterica, Salmonella paratyphi, Salmonella typhi, Salmonella typhimurium, Staphylococcus aureus, Listeria monocytogenes, Moxarella catarrhalis, Shigella boydii, Shigella dysenteriae, Shigella flexneri, Shigella sonnet, Staphylococcus epidermidis, Streptococcus pneumoniae, Streptococcus mutans, Treponema pallidum, Yersinia enterocolitica, Yersinia pestis and any species falling within the genera of any of the above species.

41. The method of Paragraph 39, wherein said method comprises inhibiting said activity or reducing said amount of a gene product in an organism other than E. coli.

42. The method of Paragraph 39, wherein said gene product is present in an organism other than E. coli.

43. The method of Paragraph 39, wherein said gene product comprises a polypeptide comprising a sequence selected from the group consisting of SEQ ID NOs.: 3801-3805, 4861-5915, 10013-14110.

44. A method for identifying a compound which influences the activity of a gene product required for proliferation, said gene product comprising a gene product whose expression is inhibited by an antisense nucleic acid comprising a nucleotide sequence selected from the group consisting of SEQ ID NOs.: 8-3795, said method comprising:

contacting said gene product with a candidate compound; and

determining whether said compound influences the activity of said gene product.

45. The method of Paragraph 44, wherein said gene product is from an organism selected from the group consisting of Anaplasma marginale, Aspergillus fumigatus, Bacillus anthracis, Bacterioides fragilis Bordetella pertussis, Burkholderia cepacia, Campylobacter jejuni, Candida albicans, Candida glabrata (also called Torulopsis glabrata), Candida tropicalis, Candida parapsilosis, Candida guilliermondii, Candida krusei, Candida kefyr (also called Candida pseudotropicalis), Candida dubliniensis, Chlamydia pneumoniae, Chlamydia trachomatus, Clostridium botulinum, Clostridium difficile, Clostridium perfringens, Coccidiodes immitis, Corynebacterium diptheriae, Cryptococcus neoformans, Enterobacter cloacae, Enterococcus faecalis, Enterococcus faecium, Escherichia coli, Haemophilus influenzae, Helicobacter pylori, Histoplasma capsulatum, Klebsiella pneumoniae, Listeria monocytogenes, Mycobacterium leprae, Mycobacterium tuberculosis, Neisseria gonorrhoeae, Neisseria meningitidis, Nocardia asteroides, Pasteurella haemolytica, Pasteurella multocida, Pneumocystis carinii, Proteus vulgaris, Pseudomonas aeruginosa, Salmonella bongori, Salmonella cholerasuis, Salmonella enterica, Salmonella paratyphi, Salmonella typhi, Salmonella typhimurium, Staphylococcus aureus, Listeria monocytogenes, Moxarella catarrhalis, Shigella boydii, Shigella dysenteriae, Shigella flexneri, Shigella sonnei, Staphylococcus epidermidis, Streptococcus pneumoniae, Streptococcus mutans, Treponema pallidum, Yersinia enterocolitica, Yersinia pestis and any species falling within the genera of any of the above species.

46. The method of Paragraph 44, wherein said gene product is from an organism other than E coli.

47. The method of Paragraph 44, wherein said gene product is a polypeptide and said activity is an enzymatic activity.

48. The method of Paragraph 44, wherein said gene product is a polypeptide and said activity is a carbon compound catabolism activity.

49. The method of Paragraph 44, wherein said gene product is a polypeptide and said activity is a biosynthetic activity.

50. The method of Paragraph 44, wherein said gene product is a polypeptide and said activity is a transporter activity.

51. The method of Paragraph 44, wherein said gene product is a polypeptide and said activity is a transcriptional activity.

52. The method of Paragraph 44, wherein said gene product is a polypeptide and said activity is a DNA replication activity.

53. The method of Paragraph 44, wherein said gene product is a polypeptide and said activity is a cell division activity.

54. The method of Paragraph 44, wherein said gene product is an RNA.

55. The method of Paragraph 44, wherein said gene product is a polypeptide comprising an amino acid sequence selected from the group consisting of SEQ ID NOs.: 3801-3805, 4861-5915, 10013-14110.

56. A compound identified using the method of Paragraph 44.

57. A method for identifying a compound or nucleic acid having the ability to reduce the activity or level of a gene product required for proliferation, said gene product comprising a gene product whose activity or expression is inhibited by an antisense nucleic acid comprising a nucleotide sequence selected from the group consisting of SEQ ID NOs.: 8-3795, said method comprising:

(a) contacting a target gene or RNA encoding said gene product with a candidate compound or nucleic acid; and

(b) measuring an activity of said target.

58. The method of Paragraph 57, wherein said target gene or RNA is from an organism selected from the group consisting of Anaplasma marginale, Aspergillus fumigatus, Bacillus anthracis, Bacterioides fragilis Bordetella pertussis, Burkholderia cepacia, Campylobacter jejuni, Candida albicans, Candida glabrata (also called Torulopsis glabrata), Candida tropicalis, Candida parapsilosis, Candida guilliermondii, Candida krusei, Candida kefyr (also called Candida pseudotropicalis), Candida dubliniensis, Chlamydia pneumoniae, Chlamydia trachomatus, Clostridium botulinum, Clostridium difficile, Clostridium perfringens, Coccidiodes immitis, Corynebacterium diptheriae, Cryptococcus neoformans, Enterobacter cloacae, Enterococcus faecalis, Enterococcus faecium, Escherichia coli, Haemophilus influenzae, Helicobacter pylori, Histoplasma capsulatum, Klebsiella pneumoniae, Listeria monocytogenes, Mycobacterium leprae, Mycobacterium tuberculosis, Neisseria gonorrhoeae, Neisseria meningitidis, Nocardia asteroides, Pasteurella haemolytica, Pasteurella multocida, Pneumocystis carinii, Proteus vulgaris, Pseudomonas aeruginosa, Salmonella bongori, Salmonella cholerasuis, Salmonella enterica, Salmonella paratyphi, Salmonella typhi, Salmonella typhimurium, Staphylococcus aureus, Listeria monocytogenes, Moxarella catarrhalis, Shigella boydii, Shigella dysenteriae, Shigella flexneri, Shigella sonnei, Staphylococcus epidermidis, Streptococcus pneumoniae, Streptococcus mutans, Treponema pallidum, Yersinia enterocolitica, Yersinia pestis and any species falling within the genera of any of the above species.

59. The method of Paragraph 57, wherein said target gene or RNA is from an organism other than E. coli.

60. The method of Paragraph 57, wherein said gene product is from an organism other than E. coli.

61. The method of Paragraph 57, wherein said target is a messenger RNA molecule and said activity is translation of said messenger RNA.

62. The method of Paragraph 57, wherein said target is a messenger RNA molecule and said activity is transcription of a gene encoding said messenger RNA.

63. The method of Paragraph 57, wherein said target is a gene and said activity is transcription of said gene.

64. The method of Paragraph 57, wherein said target is a nontranslated RNA and said activity is processing or folding of said nontranslated RNA or assembly of said nontranslated RNA into a protein/RNA complex.

65. The method of Paragraph 57, wherein said target is a messenger RNA molecule encoding a polypeptide comprising an amino acid sequence selected from the group consisting of SEQ ID NOs.: 3801-3805, 4861-5915, 10013-14110.

66. The method of Paragraph 57, wherein said target comprises a nucleic acid selected from the group consisting of SEQ ID NOS.: 3796-3800, 3806-4860, 5916-10012.

67. A compound or nucleic acid identified using the method of Paragraph 57.

68. A method for identifying a compound which reduces the activity or level of a gene product required for proliferation of a cell, wherein the activity or expression of said gene product is inhibited by an antisense nucleic acid comprising a nucleotide sequence selected from the group consisting of SEQ ID NOs.: 8-3795, said method comprising the steps of:

(a) providing a sublethal level of an antisense nucleic acid comprising a nucleotide sequence complementary to a nucleic acid comprising a nucleotide sequence encoding said gene product in a cell to reduce the activity or amount of said gene product in said cell, thereby producing a sensitized cell;

(b) contacting said sensitized cell with a compound; and

(c) determining the degree to which said compound inhibits proliferation of said sensitized cell relative to a cell which does not contain said antisense nucleic acid.

69. The method of Paragraph 68, wherein said determining step comprises determining whether said compound inhibits the growth of said sensitized cell to a greater extent than said compound inhibits the growth of a nonsensitized cell.

70. The method of Paragraph 68, wherein said cell is a Gram positive bacterium.

71. The method of Paragraph 68, wherein said Gram positive bacterium is selected from the group consisting of Staphylococcus species, Streptococcus species, Enterococcus species, Mycobacterium species, Clostridium species, and Bacillus species.

72. The method of Paragraph 68, wherein said bacterium is Staphylococcus aureus.

73. The method of Paragraph 72, wherein said Staphylococcus species is coagulase negative.

74. The method of Paragraph 72, wherein said bacterium is selected from the group consisting of Staphylococcus aureus RN450 and Staphylococcus aureus RN4220.

75. The method of Paragraph 68, wherein said cell is an organism selected from the group consisting of Anaplasma marginale, Aspergillus fumigatus, Bacillus anthracis, Bacterioides fragilis Bordetella pertussis, Burkholderia cepacia, Campylobacter jejuni, Candida albicans, Candida glabrata (also called Torulopsis glabrata), Candida tropicalis, Candida parapsilosis, Candida guilliermondii, Candida krusei, Candida kefyr (also called Candida pseudotropicalis), Candida dubliniensis, Chlamydia pneumoniae, Chlamydia trachomatus, Clostridium botulinum, Clostridium difficile, Clostridium perfringens, Coccidiodes immitis, Corynebacterium diptheriae, Cryptococcus neoformans, Enterobacter cloacae, Enterococcus faecalis, Enterococcus faecium, Escherichia coli, Haemophilus influenzae, Helicobacter pylori, Histoplasma capsulatum, Klebsiella pneumoniae, Listeria monocytogenes, Mycobacterium leprae, Mycobacterium tuberculosis, Neisseria gonorrhoeae, Neisseria meningitidis, Nocardia asteroides, Pasteurella haemolytica, Pasteurella multocida, Pneumocystis carinii, Proteus vulgaris, Pseudomonas aeruginosa, Salmonella bongori, Salmonella cholerasuis, Salmonella enterica, Salmonella paratyphi, Salmonella typhi, Salmonella typhimurium, Staphylococcus aureus, Listeria monocytogenes, Moxarella catarrhalis, Shigella boydii, Shigella dysenteriae, Shigella flexneri, Shigella sonnei, Staphylococcus epidermidis, Streptococcus pneumoniae, Streptococcus mutans, Treponema pallidum, Yersinia enterocolitica, Yersinia pestis and any species falling within the genera of any of the above species.

76. The method of Paragraph 68, wherein said cell is not an E. coli cell.

77. The method of Paragraph 68, wherein said gene product is from an organism other than E. coli.

78. The method of Paragraph 68, wherein said antisense nucleic acid is transcribed from an inducible promoter.

79. The method of Paragraph 68, further comprising the step of contacting said cell with a concentration of inducer which induces transcription of said antisense nucleic acid to a sublethal level.

80. The method of Paragraph 68, wherein growth inhibition is measured by monitoring optical density of a culture growth solution.

81. The method of Paragraph 68, wherein said gene product is a polypeptide.

82. The method of Paragraph 81, wherein said polypeptide comprises an amino acid sequence selected from the group consisting of SEQ ID NOs.: 3801-3805, 4861-5915, 10013-14110.

83. The method of Paragraph 68, wherein said gene product is an RNA.

84. The method of Paragraph 68, wherein nucleic acid encoding said gene product comprises a nucleotide sequence selected from the group consisting of SEQ ID NOS.: 3796-3800, 3806-4860, 5916-10012.

85. A compound identified using the method of Paragraph 68.

86. A method for inhibiting cellular proliferation comprising introducing an effective amount of a compound with activity against a gene whose activity or expression is inhibited by an antisense nucleic acid comprising a nucleotide sequence selected from the group consisting of SEQ ID NOs.: 8-3795 or a compound with activity against the product of said gene into a population of cells expressing said gene.

87. The method of Paragraph 86, wherein said compound is an antisense nucleic acid comprising a nucleotide sequence selected from the group consisting of SEQ ID NOs.: 8-3795, or a proliferation-inhibiting portion thereof.

88. The method of Paragraph 86, wherein said proliferation inhibiting portion of one of SEQ ID NOs.: 8-3795 is a fragment comprising at least 10, at least 20, at least 25, at least 30, at least 50 or more than 51 consecutive nucleotides of one of SEQ ID NOs.: 8-3795.

89. The method of Paragraph 86, wherein said population is a population of Gram positive bacteria.

90. The method of Paragraph 89, wherein said population of Gram positive bacteria is selected from the group consisting of a population of Staphylococcus species, Streptococcus species, Enterococcus species, Mycobacterium species, Clostridium species, and Bacillus species.

91. The method of Paragraph 86, wherein said population is a population of Staphylococcus aureus.

92. The method of Paragraph 91, wherein said population is a population of a bacterium selected from the group consisting of Staphylococcus aureus RN450 and Staphylococcus aureus RN4220.

93. The method of Paragraph 86, wherein said population is a population of a bacterium selected from the group consisting of Anaplasma marginale, Aspergillus fumigatus, Bacillus anthracis, Bacterioides fragilis Bordetella pertussis, Burkholderia cepacia, Campylobacter jejuni, Candida albicans, Candida glabrata (also called Torulopsis glabrata), Candida tropicalis, Candida parapsilosis, Candida guilliermondii, Candida krusei, Candida kefyr (also called Candida pseudotropicalis), Candida dubliniensis, Chlamydia pneumoniae, Chlamydia trachomatus, Clostridium botulinum, Clostridium difficile, Clostridium perfringens, Coccidiodes immitis, Corynebacterium diptheriae, Cryptococcus neoformans, Enterobacter cloacae, Enterococcus faecalis, Enterococcus faecium, Escherichia coli, Haemophilus influenzae, Helicobacter pylori, Histoplasma capsulatum, Klebsiella pneumoniae, Listeria monocytogenes, Mycobacterium leprae, Mycobacterium tuberculosis, Neisseria gonorrhoeae, Neisseria meningitidis, Nocardia asteroides, Pasteurella haemolytica, Pasteurella multocida, Pneumocystis carinii, Proteus vulgaris, Pseudomonas aeruginosa, Salmonella bongori, Salmonella cholerasuis, Salmonella enterica, Salmonella paratyphi, Salmonella typhi, Salmonella typhimurium, Staphylococcus aureus, Listeria monocytogenes, Moxarella catarrhalis, Shigella boydii, Shigella dysenteriae, Shigella flexneri, Shigella sonnei, Staphylococcus epidermidis, Streptococcus pneumoniae, Streptococcus mutans, Treponema pallidum, Yersinia enterocolitica, Yersinia pestis and any species falling within the genera of any of the above species.

94. The method of Paragraph 86, wherein said population is a population of an organism other than E. coli.

95. The method of Paragraph 86, wherein said product of said gene is from an organism other than E. coli.

96. The method of Paragraph 86, wherein said gene encodes a polypeptide comprising an amino acid sequence selected from the group consisting of SEQ ID NOs.: 3801-3805,4861-5915, 10013-14110.

97. The method of Paragraph 86, wherein said gene comprises a nucleotide sequence selected from the group consisting of SEQ ID NOS.: 3796-3800, 3806-4860, 5916-10012.

98. A composition comprising an effective concentration of an antisense nucleic acid comprising a nucleotide sequence selected from the group consisting of SEQ ID NOs.: 8-3795, or a proliferation-inhibiting portion thereof in a pharmaceutically acceptable carrier.

99. The composition of Paragraph 98, wherein said proliferation-inhibiting portion of one of SEQ ID NOs.: 8-3795 comprises at least 20, at least 25, at least 30, at least 50 or more than 50 consecutive nucleotides of one of SEQ ID NOs.: 8-3795.

100. A method for inhibiting the activity or expression of a gene in an operon required for proliferation wherein the activity or expression of at least one gene in said operon is inhibited by an antisense nucleic acid comprising a sequence selected from the group consisting of SEQ ID NOs.: 8-3795, said method comprising contacting a cell in a cell population with an antisense nucleic acid complementary to at least a portion of said operon.

101. The method of Paragraph 100, wherein said antisense nucleic acid comprises a nucleotide sequence selected from the group consisting of SEQ ID NOs.: 8-3795 or a proliferation-inhibiting portion thereof.

102. The method of Paragraph 100, wherein said cell is selected from the group consisting of Anaplasma marginale, Aspergillus fumigatus, Bacillus anthracis, Bacterioides fragilis Bordetella pertussis, Burkholderia cepacia, Campylobacter jejuni, Candida albicans, Candida glabrata (also called Torulopsis glabrata), Candida tropicalis, Candida parapsilosis, Candida guilliermondii, Candida krusei, Candida kefyr (also called Candida pseudotropicalis), Candida dubliniensis, Chlamydia pneumoniae, Chlamydia trachomatus, Clostridium botulinum, Clostridium difficile, Clostridium perfringens, Coccidiodes immitis, Corynebacterium diptheriae, Cryptococcus neoformans, Enterobacter cloacae, Enterococcus faecalis, Enterococcus faecium, Escherichia coli, Haemophilus influenzae, Helicobacter pylori, Histoplasma capsulatum, Klebsiella pneumoniae, Listeria monocytogenes, Mycobacterium leprae, Mycobacterium tuberculosis, Neisseria gonorrhoeae, Neisseria meningitidis, Nocardia asteroides, Pasteurella haemolytica, Pasteurella multocida, Pneumocystis carinii, Proteus vulgaris, Pseudomonas aeruginosa, Salmonella bongori, Salmonella cholerasuis, Salmonella enterica, Salmonella paratyphi, Salmonella typhi, Salmonella typhimurium, Staphylococcus aureus, Listeria monocytogenes, Moxarella catarrhalis, Shigella boydii, Shigella dysenteriae, Shigella flexneri, Shigella sonnei, Staphylococcus epidermidis, Streptococcus pneumoniae, Streptococcus mutans, Treponema pallidum, Yersinia enterocolitica, Yersinia pestis and any species falling within the genera of any of the above species.

103. The method of Paragraph 100, wherein said cell is not an E. coli cell.

104. The method of Paragraph 100, wherein said gene is from an organism other than E. coli.

105. The method of Paragraph 100, wherein said cell is contacted with said antisense nucleic acid by introducing a plasmid which expresses said antisense nucleic acid into said cell population.

106. The method of Paragraph 100, wherein said cell is contacted with said antisense nucleic acid by introducing a phage which encodes said antisense nucleic acid into said cell population.

107. The method of Paragraph 100, wherein said cell is contacted with said antisense nucleic acid by expressing said antisense nucleic acid from the chromosome of cells in said cell population.

108. The method of Paragraph 100, wherein said cell is contacted with said antisense nucleic acid by introducing a promoter adjacent to a chromosomal copy of said antisense nucleic acid such that said promoter directs the transcription of said antisense nucleic acid.

109. The method of Paragraph 100, wherein said cell is contacted with said antisense nucleic acid by introducing a retron which expresses said antisense nucleic acid into said cell population.

110. The method of Paragraph 100, wherein said cell is contacted with said antisense nucleic acid by introducing a ribozyme into said cell-population, wherein a binding portion of said ribozyme comprises said antisense nucleic acid.

111. The method of Paragraph 100, wherein said cell is contacted with said antisense nucleic acid by introducing a liposome comprising said antisense nucleic acid into said cell.

112. The method of Paragraph 100, wherein said cell is contacted with said antisense nucleic acid by electroporation of said antisense nucleic acid into said cell.

113. The method of Paragraph 100, wherein said antisense nucleic acid is a fragment comprising at least 10, at least 20, at least 25, at least 30, at least 50 or more than 50 consecutive nucleotides of one of SEQ ID NOs.: 8-3795.

114. The method of Paragraph 100 wherein said antisense nucleic acid is a synthetic oligonucleotide.

115. The method of Paragraph 100, wherein said gene comprises a nucleotide sequence selected from the group consisting of SEQ ID NOS.: 3796-3800, 3806-4860, 5916-10012.

116. A method for identifying a gene which is required for proliferation of a cell comprising:

(a) contacting a cell with an antisense nucleic acid comprising a nucleotide sequence selected from the group consisting of SEQ ID NOs.: 8-3795, wherein said cell is a cell other than the organism from which said nucleic acid was obtained;

(b) determining whether said nucleic acid inhibits proliferation of said cell; and

(c) identifying the gene in said cell which encodes the mRNA which is complementary to said antisense nucleic acid or a portion thereof.

117. The method of Paragraph 116, wherein said cell is selected from the group consisting of Staphylococcus species, Streptococcus species, Enterococcus species, Mycobacterium species, Clostridium species, and Bacillus species.

118. The method of Paragraph 116 wherein said cell is selected from the group consisting of Anaplasma marginale, Aspergillus fumigatus, Bacillus anthracis, Bacterioides fragilis Bordetella pertussis, Burkholderia cepacia, Campylobacter jejuni, Candida albicans, Candida glabrata (also called Torulopsis glabrata), Candida tropicalis, Candida parapsilosis, Candida guilliermondii, Candida krusei, Candida kefyr (also called Candida pseudotropicalis), Candida dubliniensis, Chlamydia pneumoniae, Chlamydia trachomatus, Clostridium botulinum, Clostridium difficile, Clostridium perfringens, Coccidiodes immitis, Corynebacterium diptheriae, Cryptococcus neoformans, Enterobacter cloacae, Enterococcus faecalis, Enterococcus faecium, Escherichia coli, Haemophilus influenzae, Helicobacter pylori, Histoplasma capsulatum, Klebsiella pneumoniae, Listeria monocytogenes, Mycobacterium leprae, Mycobacterium tuberculosis, Neisseria gonorrhoeae, Neisseria meningitidis, Nocardia asteroides, Pasteurella haemolytica, Pasteurella multocida, Pneumocystis carinii, Proteus vulgaris, Pseudomonas aeruginosa, Salmonella bongori, Salmonella cholerasuis, Salmonella enterica, Salmonella paratyphi, Salmonella typhi, Salmonella typhimurium, Staphylococcus aureus, Listeria monocytogenes, Moxarella catarrhalis, Shigella boydii, Shigella dysenteriae, Shigella flexneri, Shigella sonnei, Staphylococcus epidermidis, Streptococcus pneumoniae, Streptococcus mutans, Treponema pallidum, Yersinia enterocolitica, Yersinia pestis and any species falling within the genera of any of the above species.

119. The method of Paragraph 116, wherein said cell is not E. coli.

120. The method of Paragraph 116, further comprising operably linking said antisense nucleic acid to a promoter which is functional in said cell, said promoter being included in a vector, and introducing said vector into said cell.

121. A method for identifying a compound having the ability to inhibit proliferation of a cell comprising:

(a) identifying a homolog of a gene or gene product whose activity or level is inhibited by a nucleic acid comprising a nucleotide sequence selected from the group consisting of SEQ ID NOs. 8-3795 in a test cell, wherein said test cell is not the cell from which said nucleic acid was obtained;

(b) identifying an inhibitory nucleic acid sequence which inhibits the activity of said homolog in said test cell;

(c) contacting said test cell with a sublethal level of said inhibitory nucleic acid, thus sensitizing said cell;

(d) contacting the sensitized cell of step (c) with a compound; and

(e) determining the degree to which said compound inhibits proliferation of said sensitized cell relative to a cell which does not contain said inhibitory nucleic acid.

122. The method of Paragraph 121, wherein said determining step comprises determining whether said compound inhibits proliferation of said sensitized test cell to a greater extent than said compound inhibits proliferation of a nonsensitized test cell.

123. The method of Paragraph 121, wherein step (a) comprises identifying a nucleic acid homologous to a gene or gene product whose activity or level is inhibited by a nucleic acid selected from the group consisting of SEQ ID NOs. 8-3795 or a nucleic acid encoding a homologous polypeptide to a polypeptide whose activity or level is inhibited by a nucleic acid selected from the group consisting of SEQ ID NOs. 8-3795 by using an algorithm selected from the group consisting of BLASTN version 2.0 with the default parameters and FASTA version 3.0t78 algorithm with the default parameters to identify said homologous nucleic acid or said nucleic acid encoding a homologous polypeptide in a database.

124. The method of Paragraph 121 wherein said step (a) comprises identifying a homologous nucleic acid or a nucleic acid comprising a sequence of nucleotides encoding a homologous polypeptide by identifying nucleic acids which hybridize to said nucleic acid selected from the group consisting of SEQ ID NOs. 8-3795 or the complement of said nucleic acid selected from the group consisting of SEQ ID NOs. 8-3795.

125. The method of Paragraph 121 wherein step (a) comprises expressing a nucleic acid selected from the group consisting of SEQ ID NOs. 8-3795 in said test cell.

126. The method of Paragraph 121, wherein step (a) comprises identifying a homologous nucleic acid or a nucleic acid encoding a homologous polypeptide in a test cell selected from the group consisting of Anaplasma marginale, Aspergillus fumigatus, Bacillus anthracis, Bacterioides fragilis Bordetella pertussis, Burkholderia cepacia, Campylobacter jejuni, Candida albicans, Candida glabrata (also called Torulopsis glabrata), Candida tropicalis, Candida parapsilosis, Candida guilliermondii, Candida krusei, Candida kefyr (also called Candida pseudotropicalis), Candida dubliniensis, Chlamydia pneumoniae, Chlamydia trachomatus, Clostridium botulinum, Clostridium difficile, Clostridium perfringens, Coccidiodes immitis, Corynebacterium diptheriae, Cryptococcus neoformans, Enterobacter cloacae, Enterococcus faecalis, Enterococcus faecium, Escherichia coli, Haemophilus influenzae, Helicobacter pylori, Histoplasma capsulatum, Klebsiella pneumoniae, Listeria monocytogenes, Mycobacterium leprae, Mycobacterium tuberculosis, Neisseria gonorrhoeae, Neisseria meningitidis, Nocardia asteroides, Pasteurella haemolytica, Pasteurella multocida, Pneumocystis carinii, Proteus vulgaris, Pseudomonas aeruginosa, Salmonella bongori, Salmonella cholerasuis, Salmonella enterica, Salmonella paratyphi, Salmonella typhi, Salmonella typhimurium, Staphylococcus aureus, Listeria monocytogenes, Moxarella catarrhalis, Shigella boydii, Shigella dysenteriae, Shigella flexneri, Shigella sonnei, Staphylococcus epidermidis, Streptococcus pneumoniae, Streptococcus mutans, Treponema pallidum, Yersinia enterocolitica, Yersinia pestis and any species falling within the genera of any of the above species.

127. The method of Paragraph 121, wherein step (a) comprises identifying a homologous nucleic acid or a nucleic acid encoding a homologous polypeptide in a test cell other than E coli.

128. The method of Paragraph 121, wherein said inhibitory nucleic acid is an antisense nucleic acid.

129. The method of Paragraph 121, wherein said inhibitory nucleic acid comprises an antisense nucleic acid to a portion of said homolog.

130. The method of Paragraph 121, wherein said inhibitory nucleic acid comprises an antisense nucleic acid to a portion of the operon encoding said homolog.

131. The method of Paragraph 121, wherein the step of contacting the cell with a sublethal level of said inhibitory nucleic acid comprises directly contacting the surface of said cell with said inhibitory nucleic acid.

132. The method of Paragraph 121, wherein the step of contacting the cell with a sublethal level of said inhibitory nucleic acid comprises transcribing an antisense nucleic acid complementary to at least a portion of the RNA transcribed from said homolog in said cell.

133. The method of Paragraph 121, wherein said gene product comprises a polypeptide comprising an amino acid sequence selected from the group consisting of SEQ ID NOs.: 3801-3805, 4861-5915, 10013-14110.

134. The method of Paragraph 121, wherein said gene comprises a nucleotide sequence selected from the group consisting of SEQ ID NOS.: 3796-3800, 3806-4860, 5916-10012.

135. A compound identified using the method of Paragraph 121.

136. A method of identifying a compound having the ability to inhibit proliferation comprising:

(a) contacting a test cell with a sublethal level of a nucleic acid comprising a nucleotide sequence selected from the group consisting of SEQ ID NOs. 8-3795 or a portion thereof which inhibits the proliferation of the cell from which said nucleic acid was obtained, thus sensitizing said test cell;

(b) contacting the sensitized test cell of step (a) with a compound; and

(c) determining the degree to which said compound inhibits proliferation of said sensitized test cell relative to a cell which does not contain said nucleic acid.

137. The method of Paragraph 136, wherein said determining step comprises determining whether said compound inhibits proliferation of said sensitized test cell to a greater extent than said compound inhibits proliferation of a nonsensitized test cell.

138. A compound identified using the method of Paragraph 136.

139. The method of Paragraph 136, wherein said test cell is selected from the group consisting of Anaplasma marginale, Aspergillus fumigatus, Bacillus anthracis, Bacterioides fragilis Bordetella pertussis, Burkholderia cepacia, Campylobacter jejuni, Candida albicans, Candida glabrata (also called Torulopsis glabrata), Candida tropicalis, Candida parapsilosis, Candida guilliermondii, Candida krusei, Candida kefyr (also called Candida pseudotropicalis), Candida dubliniensis, Chlamydia pneumoniae, Chlamydia trachomatus, Clostridium botulinum, Clostridium difficile, Clostridium perfringens, Coccidiodes immitis, Corynebacterium diptheriae, Cryptococcus neoformans, Enterobacter cloacae, Enterococcus faecalis, Enterococcus faecium, Escherichia coli, Haemophilus influenzae, Helicobacter pylori, Histoplasma capsulatum, Klebsiella pneumoniae, Listeria monocytogenes, Mycobacterium leprae, Mycobacterium tuberculosis, Neisseria gonorrhoeae, Neisseria meningitidis, Nocardia asteroides, Pasteurella haemolytica, Pasteurella multocida, Pneumocystis carinii, Proteus vulgaris, Pseudomonas aeruginosa, Salmonella bongori, Salmonella cholerasuis, Salmonella enterica, Salmonella paratyphi, Salmonella typhi, Salmonella typhimurium, Staphylococcus aureus, Listeria monocytogenes, Moxarella catarrhalis, Shigella boydii, Shigella dysenteriae, Shigella flexneri, Shigella sonnei, Staphylococcus epidermidis, Streptococcus pneumoniae, Streptococcus mutans, Treponema pallidum, Yersinia enterocolitica, Yersinia pestis and any species falling within the genera of any of the above species.

140. The method of Paragraph 136, wherein the test cell is not E. coli.

141. A method for identifying a compound having activity against a biological pathway required for proliferation comprising:

(a) sensitizing a cell by providing a sublethal level of an antisense nucleic acid complementary to a nucleic acid encoding a gene product required for proliferation, wherein the activity or expression of said gene product is inhibited by an antisense nucleic acid comprising a nucleotide sequence selected from the group consisting of SEQ ID NOs.: 8-3795, in said cell to reduce the activity or amount of said gene product;

(b) contacting the sensitized cell with a compound; and

(c) determining the degree to which said compound inhibits the growth of said sensitized cell relative to a cell which does not contain said antisense nucleic acid.

142. The method of Paragraph 141, wherein said determining step comprises determining whether said compound inhibits the growth of said sensitized cell to a greater extent than said compound inhibits the growth of a nonsensitized cell.

143. The method of Paragraph 141, wherein said cell is selected from the group consisting of bacterial cells, fungal cells, plant cells, and animal cells.

144. The method of Paragraph 141, wherein said cell is a Gram positive bacterium.

145. The method of Paragraph 144, wherein said Gram positive bacterium is selected from the group consisting of Staphylococcus species, Streptococcus species, Enterococcus species, Mycobacterium species, Clostridium species, and Bacillus species.

146. The method of Paragraph 145, wherein said Gram positive bacterium is Staphylococcus aureus.

147. The method of Paragraph 146, wherein said Gram positive bacterium is selected from the group consisting of Staphylococcus aureus RN450 and Staphylococcus aureus RN4220.

148. The method of Paragraph 141, wherein said cell is selected from the group consisting of Anaplasma marginale, Aspergillus fumigatus, Bacillus anthracis, Bacterioides fragilis Bordetella pertussis, Burkholderia cepacia, Campylobacter jejuni, Candida albicans, Candida glabrata (also called Torulopsis glabrata), Candida tropicalis, Candida parapsilosis, Candida guilliermondii, Candida krusei, Candida kefyr (also called Candida pseudotropicalis), Candida dubliniensis, Chlamydia pneumoniae, Chlamydia trachomatus, Clostridium botulinum, Clostridium difficile, Clostridium perfringens, Coccidiodes immitis, Corynebacterium diptheriae, Cryptococcus neoformans, Enterobacter cloacae, Enterococcus faecalis, Enterococcus faecium, Escherichia coli, Haemophilus influenzae, Helicobacter pylori, Histoplasma capsulatum, Klebsiella pneumoniae, Listeria monocytogenes, Mycobacterium leprae, Mycobacterium tuberculosis, Neisseria gonorrhoeae, Neisseria meningitidis, Nocardia asteroides, Pasteurella haemolytica, Pasteurella multocida, Pneumocystis carinii, Proteus vulgaris, Pseudomonas aeruginosa, Salmonella bongori, Salmonella cholerasuis, Salmonella enterica, Salmonella paratyphi, Salmonella typhi, Salmonella typhimurium, Staphylococcus aureus, Listeria monocytogenes, Moxarella catarrhalis, Shigella boydii, Shigella dysenteriae, Shigella flexneri, Shigella sonnet, Staphylococcus epidermidis, Streptococcus pneumoniae, Streptococcus mutans, Treponema pallidum, Yersinia enterocolitica, Yersinia pestis and any species falling within the genera of any of the above species.

149. The method of Paragraph 141, wherein said cell is not an E. coli cell.

150. The method of Paragraph 141, wherein said gene product is from an organism other than E. coli.

151. The method of Paragraph 141, wherein said antisense nucleic acid is transcribed from an inducible promoter.

152. The method of Paragraph 141, further comprising contacting the cell with an agent which induces transcription of said antisense nucleic acid from said inducible promoter, wherein said antisense nucleic acid is transcribed at a sublethal level.

153. The method of Paragraph 141, wherein inhibition of proliferation is measured by monitoring the optical density of a liquid culture.

154. The method of Paragraph 141, wherein said gene product comprises a polypeptide comprising an amino acid sequence selected from the group consisting of SEQ ID NOs.: 3801-3805, 4861-5915, 10013-14110.

155. The method of Paragraph 141, wherein said nucleic acid encoding said gene product comprises a nucleotide sequence selected from the group consisting of SEQ ID NOS.: 3796-3800, 3806-4860, 5916-10012.

156. A compound identified using the method of Paragraph 141.

157. A method for identifying a compound having the ability to inhibit cellular proliferation comprising:

(a) contacting a cell with an agent which reduces the activity or level of a gene product required for proliferation of said cell, wherein said gene product is a gene product whose activity or expression is inhibited by an antisense nucleic acid comprising a nucleotide sequence selected from the group consisting of SEQ ID NOs.: 8-3795;

(b) contacting said cell with a compound; and

(c) determining whether said compound reduces proliferation of said contacted cell by acting on said gene product.

158. The method of Paragraph 157, wherein said determining step comprises determining whether said compound reduces proliferation of said contacted cell to a greater extent than said compound reduces proliferation of cells which have not been contacted with said agent.

159. The method of Paragraph 157, wherein said cell is selected from the group consisting of Anaplasma marginale, Aspergillus fumigatus, Bacillus anthracis, Bacterioides fragilis Bordetella pertussis, Burkholderia cepacia, Campylobacter jejuni, Candida albicans, Candida glabrata (also called Torulopsis glabrata), Candida tropicalis, Candida parapsilosis, Candida guilliermondii, Candida krusei, Candida kefyr (also called Candida pseudotropicalis), Candida dubliniensis, Chlamydia pneumoniae, Chlamydia trachomatus, Clostridium botulinum, Clostridium difficile, Clostridium perfringens, Coccidiodes immitis, Corynebacterium diptheriae, Cryptococcus neoformans, Enterobacter cloacae, Enterococcus faecalis, Enterococcus faecium, Escherichia coli, Haemophilus influenzae, Helicobacter pylori, Histoplasma capsulatum, Klebsiella pneumoniae, Listeria monocytogenes, Mycobacterium leprae, Mycobacterium tuberculosis, Neisseria gonorrhoeae, Neisseria meningitidis, Nocardia asteroides, Pasteurella haemolytica, Pasteurella multocida, Pneumocystis carinii, Proteus vulgaris, Pseudomonas aeruginosa, Salmonella bongori, Salmonella cholerasuis, Salmonella enterica, Salmonella paratyphi, Salmonella typhi, Salmonella typhimurium, Staphylococcus aureus, Listeria monocytogenes, Moxarella catarrhalis, Shigella boydii, Shigella dysenteriae, Shigella flexneri, Shigella sonnei, Staphylococcus epidermidis, Streptococcus pneumoniae, Streptococcus mutans, Treponema pallidum, Yersinia enterocolitica, Yersinia pestis and any species falling within the genera of any of the above species.

160. The method of Paragraph 157, wherein said cell is not an E. coli cell.

161. The method of Paragraph 157, wherein said gene product is from an organism other than E. coli.

162. The method of Paragraph 157, wherein said agent which reduces the activity or level of a gene product required for proliferation of said cell comprises an antisense nucleic acid to a gene or operon required for proliferation.

163. The method of Paragraph 157, wherein said agent which reduces the activity or level of a gene product required for proliferation of said cell comprises a compound known to inhibit growth or proliferation of a cell.

164. The method of Paragraph 157, wherein said cell contains a mutation which reduces the activity or level of said gene product required for proliferation of said cell.

165. The method of Paragraph 157, wherein said mutation is a temperature sensitive mutation.

166. The method of Paragraph 157, wherein said gene product comprises a polypeptide comprising an amino acid sequence selected from the group consisting of SEQ ID NOs.: 3801-3805, 4861-5915, 10013-14110.

167. A compound identified using the method of Paragraph 157.

168. A method for identifying the biological pathway in which a proliferation-required gene or its gene product lies, wherein said gene or gene product comprises a gene or gene product whose activity or expression is inhibited by an antisense nucleic acid comprising a sequence selected from the group consisting of SEQ ID NOs.: 8-3795, said method comprising:

(a) providing a sublethal level of an antisense nucleic acid which inhibits the activity of said proliferation-required gene or gene product in a test cell;

(b) contacting said test cell with a compound known to inhibit growth or proliferation of a cell, wherein the biological pathway on which said compound acts is known; and

(c) determining the degree to which said proliferation of said test cell is inhibited relative to a cell which was not contacted with said compound.

169. The method of Paragraph 168, wherein said determining step comprises determining whether said test cell has a substantially greater sensitivity to said compound than a cell which does not express said sublethal level of said antisense nucleic acid.

170. The method of Paragraph 168, wherein said gene product comprises a polypeptide comprising an amino acid sequence selected from the group consisting of SEQ ID NOs.: 3801-3805, 4861-5915, 10013-14110.

171. The method of Paragraph 168, wherein said test cell is selected from the group consisting of Anaplasma marginale, Aspergillus fumigatus, Bacillus anthracis, Bacterioides fragilis Bordetella pertussis, Burkholderia cepacia, Campylobacter jejuni, Candida albicans, Candida glabrata (also called Torulopsis glabrata), Candida tropicalis, Candida parapsilosis, Candida guilliermondii, Candida krusei, Candida kefyr (also called Candida pseudotropicalis), Candida dubliniensis, Chlamydia pneumoniae, Chlamydia trachomatus, Clostridium botulinum, Clostridium difficile, Clostridium perfringens, Coccidiodes immitis, Corynebacterium diptheriae, Cryptococcus neoformans, Enterobacter cloacae, Enterococcus faecalis, Enterococcus faecium, Escherichia coli, Haemophilus influenzae, Helicobacter pylori, Histoplasma capsulatum, Klebsiella pneumoniae, Listeria monocytogenes, Mycobacterium leprae, Mycobacterium tuberculosis, Neisseria gonorrhoeae, Neisseria meningitidis, Nocardia asteroides, Pasteurella haemolytica, Pasteurella multocida, Pneumocystis carinii, Proteus vulgaris, Pseudomonas aeruginosa, Salmonella bongori, Salmonella cholerasuis, Salmonella enterica, Salmonella paratyphi, Salmonella typhi, Salmonella typhimurium, Staphylococcus aureus, Listeria monocytogenes, Moxarella catarrhalis, Shigella boydii, Shigella dysenteriae, Shigella flexneri, Shigella sonnei, Staphylococcus epidermidis, Streptococcus pneumoniae, Streptococcus mutans, Treponema pallidum, Yersinia enterocolitica, Yersinia pestis and any species falling within the genera of any of the above species.

172. The method of Paragraph 168, wherein said test cell is not an E. coli cell.

173. The method of Paragraph 168, wherein said gene product is from an organism other than E. coli.

174. A method for determining the biological pathway on which a test compound acts comprising:

(a) providing a sublethal level of an antisense nucleic acid complementary to a proliferation-required nucleic acid in a first cell, wherein the activity or expression of said proliferation-required nucleic acid is inhibited by an antisense nucleic acid comprising a sequence selected from the group consisting of SEQ ID NOs.: 8-3795 and wherein the biological pathway in which said proliferation-required nucleic acid or a protein encoded by said proliferation-required nucleic acid lies is known,

(b) contacting said first cell with said test compound; and

(c) determining the degree to which said test compound inhibits proliferation of said first cell relative to a cell which does not contain said antisense nucleic acid.

175. The method of Paragraph 174, wherein said determining step comprises determining whether said first cell has a substantially greater sensitivity to said test compound than a cell which does not express said sublethal level of said antisense nucleic acid.

176. The method of Paragraph 174, further comprising:

(d) providing a sublethal level of a second antisense nucleic acid complementary to a second proliferation-required nucleic acid in a second cell, wherein said second proliferation-required nucleic acid is in a different biological pathway than said proliferation-required nucleic acid in step (a); and

(e) determining whether said second cell does not have a substantially greater sensitivity to said test compound than a cell which does not express said sublethal level of said second antisense nucleic acid, wherein said test compound is specific for the biological pathway against which the antisense nucleic acid of step (a) acts if said first cell has a substantially greater sensitivity to said test compound than said second cell.

177. The method of Paragraph 174, wherein said first cell is selected from the group consisting of Anaplasma marginale, Aspergillus fumigatus, Bacillus anthracis, Bacterioides fragilis Bordetella pertussis, Burkholderia cepacia, Campylobacter jejuni, Candida albicans, Candida glabrata (also called Torulopsis glabrata), Candida tropicalis, Candida parapsilosis, Candida guilliermondii, Candida krusei, Candida kefyr (also called Candida pseudotropicalis), Candida dubliniensis, Chlamydia pneumoniae, Chlamydia trachomatus, Clostridium botulinum, Clostridium difficile, Clostridium perfringens, Coccidiodes immitis, Corynebacterium diptheriae, Cryptococcus neoformans, Enterobacter cloacae, Enterococcus faecalis, Enterococcus faecium, Escherichia coli, Haemophilus influenzae, Helicobacter pylori, Histoplasma capsulatum, Klebsiella pneumoniae, Listeria monocytogenes, Mycobacterium leprae, Mycobacterium tuberculosis, Neisseria gonorrhoeae, Neisseria meningitidis, Nocardia asteroides, Pasteurella haemolytica, Pasteurella multocida, Pneumocystis carinii, Proteus vulgaris, Pseudomonas aeruginosa, Salmonella bongori, Salmonella cholerasuis, Salmonella enterica, Salmonella paratyphi, Salmonella typhi, Salmonella typhimurium, Staphylococcus aureus, Listeria monocytogenes, Moxarella catarrhalis, Shigella boydii, Shigella dysenteriae, Shigella flexneri, Shigella sonnei, Staphylococcus epidermidis, Streptococcus pneumoniae, Streptococcus mutans, Treponema pallidum, Yersinia enterocolitica, Yersinia pestis and any species falling within the genera of any of the above species.

178. The method of Paragraph 174, wherein said first cell is not an E. coli cell.

179. The method of Paragraph 174, wherein said proliferation-required nucleic acid is from an organism other than E. coli.

180. A purified or isolated nucleic acid comprising a sequence selected from the group consisting of SEQ ID NOs.: 8-3795.

181. A compound which interacts with a gene eorgene product whose activity or expression is inhibited by an antisense nucleic acid comprising a nucleotide sequence of one of SEQ ID NOs.: 8-3795 to inhibit proliferation.

182. The compound of Paragraph 181, wherein said gene product is a polypeptide comprising one of SEQ ID NOs.: 3801-3805, 4861-5915, 10013-14110.

183. The compound of Paragraph 181, wherein said gene comprises a nucleotide sequence selected from the group consisting of SEQ ID NOS.: 3796-3800, 3806-4860, 5916-10012.

184. A compound which interacts with a gene product whose expression is inhibited by an antisense nucleic acid comprising a nucleotide sequence of one of SEQ ID NOs.: 8-3795 to inhibit proliferation.

185. A method for manufacturing an antibiotic comprising the steps of:

screening one or more candidate compounds to identify a compound that reduces the activity or level of a gene product required for proliferation, said gene product comprising a gene product whose activity or expression is inhibited by an antisense nucleic acid comprising a nucleotide sequence selected from the group consisting of SEQ ID NOs.: 8-3795; and

manufacturing the compound so identified.

186. The method of Paragraph 185, wherein said screening step comprises performing any one of the methods of Paragraphs 44, 68, 121, 136, 141, and 157.

187. The method of Paragraph 185, wherein said gene product is a polypeptide comprising one of SEQ ID NOs:3801-3805, 4861-5915, 10013-14110.

188. A method for inhibiting proliferation of a cell in a subject comprising administering an effective amount of a compound that reduces the activity or level of a gene product required for proliferation of said cell, said gene product comprising a gene product whose activity or expression is inhibited by an antisense nucleic acid comprising a nucleotide sequence selected from the group consisting of SEQ ID NOs.: 8-3795 to said subject.

189. The method of Paragraph 188 wherein said subject is selected from the group consisting of vertebrates, mammals, avians, and human beings.

190. The method of Paragraph 188, wherein said gene product comprises a polypeptide comprising an amino acid sequence selected from the group consisting of SEQ ID NOs.: 3801-3805, 4861-5915, 10013-14110.

191. The method of Paragraph 188, wherein said cell is selected from the group consisting of Anaplasma marginale, Aspergillus fumigatus, Bacillus anthracis, Bacterioides fragilis Bordetella pertussis, Burkholderia cepacia, Campylobacter jejuni, Candida albicans, Candida glabrata (also called Torulopsis glabrata), Candida tropicalis, Candida parapsilosis, Candida guilliermondii, Candida krusei, Candida kefyr (also called Candida pseudotropicalis), Candida dubliniensis, Chlamydia pneumoniae, Chlamydia trachomatus, Clostridium botulinum, Clostridium difficile, Clostridium perfringens, Coccidiodes immitis, Corynebacterium diptheriae, Cryptococcus neoformans, Enterobacter cloacae, Enterococcus faecalis, Enterococcus faecium, Escherichia coli, Haemophilus influenzae, Helicobacter pylori, Histoplasma capsulatum, Klebsiella pneumoniae, Listeria monocytogenes, Mycobacterium leprae, Mycobacterium tuberculosis, Neisseria gonorrhoeae, Neisseria meningitidis, Nocardia asteroides, Pasteurella haemolytica, Pasteurella multocida, Pneumocystis carinii, Proteus vulgaris, Pseudomonas aeruginosa, Salmonella bongori, Salmonella cholerasuis, Salmonella enterica, Salmonella paratyphi, Salmonella typhi, Salmonella typhimurium, Staphylococcus aureus, Listeria monocytogenes, Moxarella catarrhalis, Shigella boydii, Shigella dysenteriae, Shigella flexneri, Shigella sonnei, Staphylococcus epidermidis, Streptococcus pneumoniae, Streptococcus mutans, Treponema pallidum, Yersinia enterocolitica, Yersinia pestis and any species falling within the genera of any of the above species.

192. The method of Paragraph 188, wherein said cell is not E. coli.

193. The method of Paragraph 188, wherein said gene product is from an organism other than E. coli.

194. A purified or isolated nucleic acid consisting essentially of the coding sequence of one of SEQ ID NOs: 3796-3800, 3806-4860, 5916-10012.

195. A fragment of the nucleic acid of Paragraph 8, said fragment comprising at least 10, at least 20, at least 25, at least 30, at least 50 or more than 50 consecutive nucleotides of one of SEQ ID NOs: 3796-3800, 3806-4860, 5916-10012.

196. A purified or isolated nucleic acid comprising a nucleic acid having at least 70% nucleotide sequence identity to a nucleotide sequence selected from the group consisting of SEQ ID NOs.: 3796-3800, 3806-4860, 5916-10012, fragments comprising at least 25 consecutive nucleotides of SEQ ID NOs.: 3796-3800, 3806-4860, 5916-10012, the nucleotide sequences complementary to SEQ ID NOs.:3796-3800, 3806-4860, 5916-10012, and the nucleotide sequences complementary to fragments comprising at least 25 consecutive nucleotides of SEQ ID NOs.: 3796-3800, 3806-4860, 5916-10012 as determined using BLASTN version 2.0 with the default parameters.

197. The nucleic acid of Paragraph 196, wherein said nucleic acid is from an organism selected from the group consisting of Anaplasma marginale, Aspergillus fumigatus, Bacillus anthracis, Bacterioides fragilis Bordetella pertussis, Burkholderia cepacia, Campylobacter jejuni, Candida albicans, Candida glabrata (also called Torulopsis glabrata), Candida tropicalis, Candida parapsilosis, Candida guilliermondii, Candida krusei, Candida kefyr (also called Candida pseudotropicalis), Candida dubliniensis, Chlamydia pneumoniae, Chlamydia trachomatus, Clostridium botulinum, Clostridium difficile, Clostridium perfringens, Coccidiodes immitis, Corynebacterium diptheriae, Cryptococcus neoformans, Enterobacter cloacae, Enterococcus faecalis, Enterococcus faecium, Escherichia coli, Haemophilus influenzae, Helicobacter pylori, Histoplasma capsulatum, Klebsiella pneumoniae, Listeria monocytogenes, Mycobacterium leprae, Mycobacterium tuberculosis, Neisseria gonorrhoeae, Neisseria meningitidis, Nocardia asteroides, Pasteurella haemolytica, Pasteurella multocida, Pneumocystis carinii, Proteus vulgaris, Pseudomonas aeruginosa, Salmonella bongori, Salmonella cholerasuis, Salmonella enterica, Salmonella paratyphi, Salmonella typhi, Salmonella typhimurium, Staphylococcus aureus, Listeria monocytogenes, Moxarella catarrhalis, Shigella boydii, Shigella dysenteriae, Shigella flexneri, Shigella sonnei Staphylococcus epidermidis, Streptococcus pneumoniae, Streptococcus mutans, Treponema pallidum, Yersinia enterocolitica, Yersinia pestis and any species falling within the genera of any of the above species.

198. The nucleic acid of Paragraph 196, wherein said nucleic acid is from an organism other than E. coli.

199. A method of inhibiting proliferation of a cell comprising inhibiting the activity or reducing the amount of a gene product in said cell or inhibiting the activity or reducing the amount of a nucleic acid encoding said gene product in said cell, wherein said gene product is selected from the group consisting of a gene product having having at least 70% nucleotide sequence identity as determined using BLASTN version 2.0 with the default parameters to a gene product whose expression is inhibited by an antisense nucleic acid comprising a nucleotide sequence selected from the group consisting of SEQ ID NOs.: 8-3795, a gene product encoded by a nucleic acid having at least 70% nucleotide sequence identity as determined using BLASTN version 2.0 with the default parameters to a nucleic acid encoding a gene product whose expression is inhibited by an antisense nucleic acid comprising a nucleotide sequence selected from the group consisting of SEQ ID NOs:8-3795, a gene product having at least 25% amino acid identity as determined using FASTA version 3.0t78 with the default parameters to a gene product whose expression is inhibited by an antisense nucleic acid comprising a nucleotide sequence selected from the group consisting of SEQ ID NOs.: 8-3795, a gene product encoded by a nucleic acid which hybridizes to a nucleic acid comprising a nucleotide sequence selected from the croup consisting of SEQ ID NOs.: 8-3795 under stringent conditions, a gene product encoded by a nucleic acid which hybridizes to a nucleic acid comprising a nucleotide sequence selected from the group consisting of SEQ ID NOs.: 8-3795 under moderate conditions, and a gene product whose activity may be complemented by the gene product whose activity is inhibited by a nucleic acid comprising a nucleotide sequence selected from the group consisting of SEQ ID NOs: 8-3795.

200. The method of Paragraph 199, wherein said method comprises inhibiting said activity or reducing said amount of said gene product or inhibiting the activity or reducing the amount of a nucleic acid encoding said gene product in an organism selected from the group consisting of Anaplasma marginale, Aspergillus fumigatus, Bacillus anthracis, Bacterioides fragilis Bordetella pertussis, Burkholderia cepacia, Campylobacter jejuni, Candida albicans, Candida glabrata (also called Torulopsis glabrata), Candida tropicalis, Candida parapsilosis, Candida guilliermondii, Candida krusei, Candida kefyr (also called Candida pseudotropicalis), Candida dubliniensis, Chlamydia pneumoniae, Chlamydia trachomatus, Clostridium botulinum, Clostridium difficile, Clostridium perfringens, Coccidiodes immitis, Corynebacterium diptheriae, Cryptococcus neoformans, Enterobacter cloacae, Enterococcus faecalis, Enterococcus faecium, Escherichia coli, Haemophilus influenzae, Helicobacter pylori, Histoplasma capsulatum, Klebsiella pneumoniae, Listeria monocytogenes, Mycobacterium leprae, Mycobacterium tuberculosis, Neisseria gonorrhoeae, Neisseria meningitidis, Nocardia asteroides, Pasteurella haemolytica, Pasteurella multocida, Pneumocystis carinii, Proteus vulgaris, Pseudomonas aeruginosa, Salmonella bongori, Salmonella cholerasuis, Salmonella enterica, Salmonella paratyphi, Salmonella typhi, Salmonella typhimurium, Staphylococcus aureus, Listeria monocytogenes, Moxarella catarrhalis, Shigella boydii, Shigella dysenteriae, Shigella flexneri, Shigella sonnei, Staphylococcus epidermidis, Streptococcus pneumoniae, Streptococcus mutans, Treponema pallidum, Yersinia enterocolitica, Yersinia pestis and any species falling within the genera of any of the above species.

201. The method of Paragraph 199, wherein said method comprises inhibiting said activity or reducing said amount of said gene product or inhibiting the activity or reducing the amount of a nucleic acid encoding said gene product in an organism other than E. coli.

202. The method of Paragraph 199, wherein said gene product is from an organism other than E. coli.

203. The method of Paragraph 199, wherein said gene product comprises a polypeptide selected from the group consisting of a polypeptide having at least 25% amino acid identity as determined using FASTA version 3.0t78 to a polypeptide selected from the group consisting of SEQ ID NOs.: 3801-3805, 4861-5915, 10013-14110 and a polypeptide whose activity may be complemented by a polypeptide selected from the group consisting of SEQ ID NOs: 3801-3805, 4861-5915, 10013-14110.

204. The method of Paragraph 199, wherein said gene product is encoded by a nucleic acid selected from the group consisting of a nucleic acid comprising a nucleic acid having at least 70% nucleotide sequence identity as determined using BLASTN version 2.0 with the default parameters to a nucleotide sequence selected from the group consisting of SEQ ID NOS.: 3796-3800, 3806-4860, 5916-10012, a nucleic acid comprising a nucleotide sequence which hybridizes to a sequence selected from the group consisting of SEQ ID NOS.: 3796-3800, 3806-4860, 5916-10012 under stringent conditions, and a nucleic acid comprising a nucloetide sequence which hybridizes to a nucleotide sequence selected from the group consisting of SEQ ID NOS.: 3796-3800, 3806-4860, 5916-10012 under moderate condtions.

205. A method for identifying a compound which influences the activity of a gene product required for proliferation comprising:

contacting a candidate compound with a gene product selected from the group consisting of a gene product having at least 70% nucleotide sequence identity as determined using BLASTN version 2.0 with the default parameters to a gene product whose expression is inhibited by an antisense nucleic acid comprising a nucleotide sequence selected from the group consisting of SEQ ID NOs.: 8-3795, a gene product encoded by a nucleic acid having at least 70% nucleotide sequence identity as determined using BLASTN version 2.0 with the default parameters to a nucleic acid encoding a gene product whose expression is inhibited by an antisense nucleic acid comprising a nucleotide sequence selected from the group consisting of SEQ ID NOs:8-3795, a gene product having at least 25% amino acid identity as determined using FASTA version 3.0t78 with the default parameters to a gene product whose expression is inhibited by an antisense nucleic acid comprising a nucleotide sequence selected from the group consisting of SEQ ID NOs.: 8-3795, a gene product encoded by a nucleic acid comprising a nucleotide sequence which hybridizes to a nucleic acid selected from the group consisting of SEQ ID NOs.: 8-3795 under stringent ,conditions, a gene product encoded by a nucleic acid comprising a nucleotide sequence which hybridizes to a nucleic acid selected from the group consisting of SEQ ID NOs.: 8-3795 under moderate conditions, and a gene product whose activity may be complemented by the gene product whose activity is inhibited by a nucleic acid selected from the group consisting of SEQ ID NOs: 8-3795; and

determining whether said candidate compound influences the activity of said gene product.

206. The method of Paragraph 205, wherein said gene product is from an organism selected from the group consisting of Anaplasma marginale, Aspergillus fumigatus, Bacillus anthracis, Bacterioides fragilis Bordetella pertussis, Burkholderia cepacia, Campylobacter jejuni, Candida albicans, Candida glabrata (also called Torulopsis glabrata), Candida tropicalis, Candida parapsilosis, Candida guilliermondii, Candida krusei, Candida kefyr (also called Candida pseudotropicalis), Candida dubliniensis, Chlamydia pneumoniae, Chlamydia trachomatus, Clostridium botulinum, Clostridium difficile, Clostridium perfringens, Coccidiodes immitis, Corynebacterium diptheriae, Cryptococcus neoformans, Enterobacter cloacae, Enterococcus faecalis, Enterococcus faecium, Escherichia coli, Haemophilus influenzae, Helicobacter pylori, Histoplasma capsulatum, Klebsiella pneumoniae, Listeria monocytogenes, Mycobacterium leprae, Mycobacterium tuberculosis, Neisseria gonorrhoeae, Neisseria meningitidis, Nocardia asteroides, Pasteurella haemolytica, Pasteurella multocida, Pneumocystis carinii, Proteus vulgaris, Pseudomonas aeruginosa, Salmonella bongori, Salmonella cholerasuis, Salmonella enterica, Salmonella paratyphi, Salmonella typhi, Salmonella typhimurium, Staphylococcus aureus, Listeria monocytogenes, Moxarella catarrhalis, Shigella boydii, Shigella dysenteriae, Shigella flexneri, Shigella sonnei, Staphylococcus epidermidis, Streptococcus pneumoniae, Streptococcus mutans, Treponema pallidum, Yersinia enterocolitica, Yersinia pestis and any species falling within the genera of any of the above species.

207. The method of Paragraph 205, wherein said gene product is from an organism other than E. coli.

208. The method of Paragraph 205, wherein said gene product is a polypeptide selected from the group consisting of a polypeptide having at least 25% amino acid identity as determined using FASTA version 3.0t78 to a polypeptide selected from the group consisting of SEQ ID NOs.: 3801-3805, 4861-5915, 10013-14110 and a polypeptide whose activity may be complemented by a polypeptide selected from the group consisting of SEQ ID NOs: 3801-3805, 4861-5915, 10013-14110.

209. The method of Paragraph 205, wherein said gene product is encoded by a nucleic acid selected from the group consisting of a nucleic acid comprising a nucleic acid having at least 70% nucleotide sequence identity as determined using BLASTN version 2.0 with the default parameters to a nucleotide sequence selected from the group consisting of SEQ ID NOS.: 3796-3800, 3806-4860, 5916-10012, a nucleic acid which hybridizes to a sequence selected from the group consisting of SEQ ID NOS.: 3796-3800, 3806-4860, 5916-10012 under stringent conditions, and a nucleic acid which hybridizes to a sequence selected from the group consisting of SEQ ID NOS.: 3796-3800, 3806-4860, 5916-10012 under moderate condtions.

210. A compound identified using the method of Paragraph 205.

211. A method for identifying a compound or nucleic acid having the ability to reduce the activity or level of a gene product required for proliferation comprising:

(a) providing a target that is a gene or RNA, wherein said target comprises a nucleic acid that encodes a gene product selected from the group consisting of a gene product having having at least 70% nucleotide sequence identity as determined using BLASTN version 2.0 with the default parameters to a gene product whose expression is inhibited by an antisense nucleic acid comprising a nucleotide sequence selected from the group consisting of SEQ ID NOs.: 8-3795, a gene product encoded by a nucleic acid having at least 70% nucleic acid identity as determined using BLASTN version 2.0 with the default parameters to a nucleic acid encoding a gene product whose expression is inhibited by an antisense nucleic acid comprising a nucleotide sequence selected from the group consisting of SEQ ID NOs:8-3795, a gene product having at least 25% amino acid identity as determined using FASTA version 3.0t78 with the default parameters to a gene product whose expression is inhibited by an antisense nucleic acid comprising a sequence selected from the group consisting of SEQ ID NOs.: 8-3795, a gene product encoded by a nucleic acid comprising a nucleotide sequence which hybridizes to a nucleic acid selected from the group consisting of SEQ ID NOs.: 8-3795 under stringent conditions, a gene product encoded by a nucleic acid comprising a nucleotide sequence which hybridizes to a nucleic acid selected from the group consisting of SEQ ID NOs.: 8-3795 under moderate conditions, and a gene product whose activity may be complemented by the gene product whose activity is inhibited by a nucleic acid selected from the group consisting of SEQ ID NOs: 8-3795;

(b) contacting said target with a candidate compound or nucleic acid; and

(c) measuring an activity of said target.

212. The method of Paragraph 211, wherein said target gene or RNA is from an organism selected from the group consisting of Anaplasma marginale, Aspergillus fumigatus, Bacillus anthracis, Bacterioides fragilis Bordetella pertussis, Burkholderia cepacia, Campylobacter jejuni, Candida albicans, Candida glabrata (also called Torulopsis glabrata), Candida tropicalis, Candida parapsilosis, Candida guilliermondii, Candida krusei, Candida kefyr (also called Candida pseudotropicalis), Candida dubliniensis, Chlamydia pneumoniae, Chlamydia trachomatus, Clostridium botulinum, Clostridium difficile, Clostridium perfringens, Coccidiodes immitis, Corynebacterium diptheriae, Cryptococcus neoformans, Enterobacter cloacae, Enterococcus faecalis, Enterococcus faecium, Escherichia coli, Haemophilus influenzae, Helicobacter pylori, Histoplasma capsulatum, Klebsiella pneumoniae, Listeria monocytogenes, Mycobacterium leprae, Mycobacterium tuberculosis, Neisseria gonorrhoeae, Neisseria meningitidis, Nocardia asteroides, Pasteurella haemolytica, Pasteurella multocida, Pneumocystis carinii, Proteus vulgaris, Pseudomonas aeruginosa, Salmonella bongori, Salmonella cholerasuis, Salmonella enterica, Salmonella paratyphi, Salmonella typhi, Salmonella typhimurium, Staphylococcus aureus, Listeria monocytogenes, Moxarella catarrhalis, Shigella boydii, Shigella dysenteriae, Shigella flexneri, Shigella sonnei, Staphylococcus epidermidis, Streptococcus pneumoniae, Streptococcus mutans, Treponema pallidum, Yersinia enterocolitica, Yersinia pestis and any species falling within the genera of any of the above species.

213. The method of Paragraph 211, wherein said target gene or RNA is from an organism other than E. coli.

214. The method of Paragraph 211, wherein said gene product is from an organism other than E. coli.

215. The method of Paragraph 211, wherein said target is a messenger RNA molecule and said activity is translation of said messenger RNA.

216. The method of Paragraph 211, wherein said compound is a nucleic acid and said activity is translation of said gene product.

217. The method of Paragraph 211, wherein said target is a gene and said activity is transcription of said gene.

218. The method of Paragraph 211, wherein said target is a nontranslated RNA and said activity is processing or folding of said nontranslated RNA or assembly of said nontranslated RNA into a protein/RNA complex.

219. The method of Paragraph 211, wherein said target gene is a messenger RNA molecule encoding a polypeptide selected from the group consisting of a polypeptide having at least 25% amino acid identity as determined using FASTA version 3.0t78 to a polypeptide selected from the group consisting of SEQ ID NOs.: 3801-3805, 4861-5915, 10013-14110 and a polypeptide whose activity may be complemented by a polypeptide selected from the group consisting of SEQ ID NOs: 3801-3805, 4861-5915, 10013-14110.

220. The method of Paragraph 11, wherein said target gene comprises a nucleic acid selected from the group consisting of a nucleic acid comprising a nucleic acid having at least 70% nucleotide sequence identity as determined using BLASTN version 2.0 with the default parameters to a nucleotide sequence selected from the group consisting of SEQ ID NOS.: 3796-3800, 3806-4860, 5916-10012, a nucleic acid which hybridizes to a sequence selected from the group consisting of SEQ ID NOS.: 3796-3800, 3806-4860, 5916-10012 under stringent conditions, and a nucleic acid which hybridizes to a sequence selected from the group consisting of SEQ ID NOS.: 3796-3800, 3806-4860, 5916-10012 under moderate condtions.

221. A compound or nucleic acid identified using the method of Paragraph 211.

222. A method for identifying a compound which reduces the activity or level of a gene product required for proliferation of a cell comprising:

(a) providing a sublethal level of an antisense nucleic acid complementary to a nucleic acid encoding said gene product in a cell to reduce the activity or amount of said gene product in said cell, thereby producing a sensitized cell, wherein said gene product is selected from the group consisting of a gene product having having at least 70% nucleic acid identity as determined using BLASTN version 2.0 with the default parameters to a gene product whose expression is inhibited by an antisense nucleic acid comprising a nucleotide sequence selected from the group consisting of SEQ ID NOs.: 8-3795, a gene product encoded by a nucleic acid having at least 70% nucleotide sequence identity as determined using BLASTN version 2.0 with the default parameters to a nucleic acid encoding a gene product whose expression is inhibited by an antisense nucleic acid comprising a nucleotide sequence selected from the group consisting of SEQ ID NOs:8-3795, a gene product having at least 25% amino acid identity as determined using FASTA version 3.0t78 with the default parameters to a gene product whose expression is inhibited by an antisense nucleic acid comprising a nucleotide sequence selected from the group consisting of SEQ ID NOs.: 8-3795, a gene product encoded by a nucleic acid comprising a nucleotide sequence which hybridizes to a nucleic acid selected from the group consisting of SEQ ID NOs.: 8-3795 under stringent conditions, a gene product encoded by a nucleic acid comprising a nucleotide sequence which hybridizes to a nucleic acid comprising a nucleotide sequence selected from the group consisting of SEQ ID NOs.: 8-3795 under moderate conditions, and a gene product whose activity may be complemented by the gene product whose activity is inhibited by a nucleic acid selected from the group consisting of SEQ ID NOs: 8-3795;

(b) contacting said sensitized cell with a compound; and

(c) determining the degree to which said compound inhibits the growth of said sensitized cell relative to a cell which does not contain said antisense nucleic acid.

223. The method of Paragraph 222, wherein said determining step comprises determining whether said compound inhibits the growth of said sensitized cell to a greater extent than said compound inhibits the growth of a nonsensitized cell.

224. The method of Paragraph 222, wherein said sensitized cell is a Gram positive bacterium.

225. The method of Paragraph 224, wherein said Gram positive bacterium is selected from the group consisting of Staphylococcus species, Streptococcus species, Enterococcus species, Mycobacterium species, Clostridium species, and Bacillus species.

226. The method of Paragraph 225, wherein said bacterium is Staphylococcus aureus.

227. The method of Paragraph 224, wherein said Staphylococcus species is coagulase negative.

228. The method of Paragraph 226, wherein said bacterium is selected from the group consisting of Staphylococcus aureus RN450 and Staphylococcus aureus RN4220.

229. The method of Paragraph 222, wherein said sensitized cell is an organism selected from the group consisting of Anaplasma marginale, Aspergillus fumigatus, Bacillus anthracis, Bacterioides fragilis Bordetella pertussis, Burkholderia cepacia, Campylobacter jejuni, Candida albicans, Candida glabrata (also called Torulopsis glabrata), Candida tropicalis, Candida parapsilosis, Candida guilliermondii, Candida krusei, Candida kefyr (also called Candida pseudotropicalis), Candida dubliniensis, Chlamydia pneumoniae, Chlamydia trachomatus, Clostridium botulinum, Clostridium difficile, Clostridium perfringens, Coccidiodes immitis, Corynebacterium diptheriae, Cryptococcus neoformans, Enterobacter cloacae, Enterococcus faecalis, Enterococcus faecium, Escherichia coli, Haemophilus influenzae, Helicobacter pylori, Histoplasma capsulatum, Klebsiella pneumoniae, Listeria monocytogenes, Mycobacterium leprae, Mycobacterium tuberculosis, Neisseria gonorrhoeae, Neisseria meningitidis, Nocardia asteroides, Pasteurella haemolytica, Pasteurella multocida, Pneumocystis carinii, Proteus vulgaris, Pseudomonas aeruginosa, Salmonella bongori, Salmonella cholerasuis, Salmonella enterica, Salmonella paratyphi, Salmonella typhi, Salmonella typhimurium, Staphylococcus aureus, Listeria monocytogenes, Moxarella catarrhalis, Shigella boydii, Shigella dysenteriae, Shigella flexneri, Shigella sonnei, Staphylococcus epidermidis, Streptococcus pneumoniae, Streptococcus mutans, Treponema pallidum, Yersinia enterocolitica, Yersinia pestis and any species falling within the genera of any of the above species.

230. The method of Paragraph 222, wherein said cell is an organism other than E. coli.

231. The method of Paragraph 222, wherein said gene product is from an organism other than E. coli.

232. The method of Paragraph 222, wherein said antisense nucleic acid is transcribed from an inducible promoter.

233. The method of Paragraph 222, further comprising the step of contacting said cell with a concentration of inducer which induces transcription of said antisense nucleic acid to a sublethal level.

234. The method of Paragraph 222, wherein growth inhibition is measured by monitoring optical density of a culture medium.

235. The method of Paragraph 222, wherein said gene product is a polypeptide.

236. The method of Paragraph 235, wherein said polypeptide comprises a polypeptide selected from the group consisting of a polypeptide having at least 25% amino acid identity as determined using FASTA version 3.0t78 to a polypeptide selected from the group consisting of SEQ ID NOs.: 3801-3805, 4861-5915, 10013-14110 and a polypeptide whose activity may be complemented by a polypeptide selected from the group consisting of SEQ ID NOs: 3801-3805, 4861-5915, 10013-14110.

237. The method of Paragraph 222, wherein said gene product is an RNA.

238. The method of Paragraph 222, wherein said nucleic acid encoding said gene product comprises a nucleic acid selected from the group consisting of a nucleic acid comprising a nucleic acid having at least 70% nucleic acid identity as determined using BLASTN version 2.0 with the default parameters to a sequence selected from the group consisting of SEQ ID NOS.: 3796-3800, 3806-4860, 5916-10012, a nucleic acid which hybridizes to a sequence selected from the group consisting of SEQ ID NOS.: 3796-3800, 3806-4860, 5916-10012 under stringent conditions, and a nucleic acid which hybridizes to a sequence selected from the group consisting of SEQ ID NOS.: 3796-3800, 3806-4860, 5916-10012 under moderate condtions.

239. A compound identified using the method of Paragraph 222.

240. A method for inhibiting cellular proliferation comprising introducing a compound with activity against a gene product or a compound with activity against a gene encoding said gene product into a population of cells expressing said gene product, wherein said gene product is selected from the group consisting of a gene product having at least 70% nucleotide sequence identity as determined using BLASTN version 2.0 with the default parameters to a gene product whose expression is inhibited by an antisense nucleic acid comprising a nucleotide sequence selected from the group consisting of SEQ ID NOs.: 8-3795, a gene product encoded by a nucleic acid having at least 70% nucleotide sequence identity as determined using BLASTN version 2.0 with the default parameters to a nucleic acid encoding a gene product whose expression is inhibited by an antisense nucleic acid comprising a nucleotide sequence selected from the group consisting of SEQ ID NOs:8-3795, a gene product having at least 25% amino acid identity as determined using FASTA version 3.0t78 with the default parameters to a gene product whose expression is inhibited by an antisense nucleic acid comprising a nucleotide sequence selected from the group consisting of SEQ ID NOs.: 8-3795, a gene product encoded by a nucleic acid comprising a nucleotide sequence which hybridizes to a nucleic acid selected from the group consisting of SEQ ID NOs.: 8-3795 under stringent conditions, a gene product encoded by a nucleic acid comprising a nucleotide sequence which hybridizes to a nucleic acid selected from the group consisting of SEQ ID NOs.: 8-3795 under moderate conditions, and a gene product whose activity may be complemented by the gene product whose activity is inhibited by a nucleic acid selected from the group consisting of SEQ ID NOs: 8-3795.

241. The method of Paragraph 240, wherein said compound is an antisense nucleic acid comprising a nucleotide sequence selected from the group consisting of SEQ ID NOs.: 8-3795, or a proliferation-inhibiting portion thereof.

242. The method of Paragraph 240, wherein said proliferation inhibiting portion of one of SEQ ID NOs.: 8-3795 is a fragment comprising at least 10, at least 20, at least 25, at least 30, at least 50 or more than 51 consecutive nucleotides of one of SEQ ID NOs.: 8-3795.

243. The method of Paragraph 240, wherein said population is a population of Gram positive bacteria.

244. The method of Paragraph 243, wherein said population of Gram positive bacteria is selected from the group consisting of a population of Staphylococcus species, Streptococcus species, Enterococcus species, Mycobacterium species, Clostridium species, and Bacillus species.

245. The method of Paragraph 243, wherein said population is a population of Staphylococcus aureus.

246. The method of Paragraph 245, wherein said population is a population of a bacterium selected from the group consisting of Staphylococcus aureus RN450 and Staphylococcus aureus RN4220.

247. The method of Paragraph 240, wherein said population is a population of a bacterium selected from the group consisting of Anaplasma marginale, Aspergillus fumigatus, Bacillus anthracis, Bacterioides fragilis Bordetella pertussis, Burkholderia cepacia, Campylobacter jejuni, Candida albicans, Candida glabrata (also called Torulopsis glabrata), Candida tropicalis, Candida parapsilosis, Candida guilliermondii, Candida krusei, Candida kefyr (also called Candida pseudotropicalis), Candida dubliniensis, Chlamydia pneumoniae, Chlamydia trachomatus, Clostridium botulinum, Clostridium difficile, Clostridium perfringens, Coccidiodes immitis, Corynebacterium diptheriae, Cryptococcus neoformans, Enterobacter cloacae, Enterococcus faecalis, Enterococcus faecium, Escherichia coli, Haemophilus influenzae, Helicobacter pylori, Histoplasma capsulatum, Klebsiella pneumoniae, Listeria monocytogenes, Mycobacterium leprae, Mycobacterium tuberculosis, Neisseria gonorrhoeae, Neisseria meningitidis, Nocardia asteroides, Pasteurella haemolytica, Pasteurella multocida, Pneumocystis carinii, Proteus vulgaris, Pseudomonas aeruginosa, Salmonella bongori, Salmonella cholerasuis, Salmonella enterica, Salmonella paratyphi, Salmonella typhi, Salmonella typhimurium, Staphylococcus aureus, Listeria monocytogenes, Moxarella catarrhalis, Shigella boydii, Shigella dysenteriae, Shigella flexneri, Shigella sonnet, Staphylococcus epidermidis, Streptococcus pneumoniae, Streptococcus mutans, Treponema pallidum, Yersinia enterocolitica, Yersinia pestis and any species falling within the genera of any of the above species.

248. The method of Paragraph 240, wherein said population is a population of an organism other than E. coli.

249. The method of Paragraph 240, wherein said product of said gene is from an organism other than E. coli.

250. The method of Paragraph 240, wherein said gene product is selected from the group consisting of a polypeptide having at least 25% amino acid identity as determined using FASTA version 3.0t78 to a polypeptide selected from the group consisting of SEQ ID NOs.: 3801-3805, 4861-5915, 10013-14110 and a polypeptide whose activity may be complemented by a polypeptide selected from the group consisting of SEQ ID NOs: 3801-3805, 4861-5915, 10013-14110.

251. The method of Paragraph 240, wherein said gene comprises a nucleic acid selected from the group consisting of a nucleic acid comprising a nucleic acid having at least 70% nucleotide sequence identity as determined using BLASTN version 2.0 with the default parameters to a nucleotide sequence selected from the group consisting of SEQ ID NOS.: 3796-3800, 3806-4860, 5916-10012, a nucleic acid comprising a nucleotide sequence which hybridizes to a nucleotide sequence selected from the group consisting of SEQ ID NOS.: 3796-3800, 3806-4860, 5916-10012 under stringent conditions, and a nucleic acid comprising a nucleotide sequence which hybridizes to a nucleotide sequence selected from the group consisting of SEQ ID NOS.: 3796-3800, 3806-4860, 5916-10012 under moderate condtions.

252. A preparation comprising an effective concentration of an antisense nucleic acid in a pharmaceutically acceptable carrier wherein said antisense nucleic acid is selected from the group consisting of a nucleic acid comprising a sequence having at least 70% nucleotide sequence identity as determined using BLASTN version 2.0 with the default parameters to a nucleotide sequence selected from the group consisting of SEQ ID NOs.: 8-3795 or a proliferation-inhibiting portion thereof, a nucleic acid comprising a nucleotide sequence which hybridizes to a nucleic acid selected from the group consisting of SEQ ID NOs.: 8-3795 under stringent conditions, and a nucleic acid comprising a nucleotide sequence which hybridizes to a nucleic acid selected from the group consisting of SEQ ID NOs.: 8-3795 under moderate conditions.

253. The preparation of Paragraph 252, wherein said proliferation-inhibiting portion of one of SEQ ID NOs.: 8-3795 comprises at least 10, at least 20, at least 25, at least 30, at least 50 or more than 50 consecutive nucleotides of one of SEQ ID NOs.: 8-3795.

254. A method for inhibiting the activity or expression of a gene in an operon which encodes a gene product required for proliferation comprising contacting a cell in a cell population with an antisense nucleic acid comprising at least a proliferation-inhibiting portion of said operon in an antisense orientation, wherein said gene product is selected from the group consisting of a gene product having at least 70% nucleotide sequence identity as determined using BLASTN version 2.0 with the default parameters to a gene product whose expression is inhibited by an antisense nucleic acid comprising a nucleotide sequence selected from the group consisting of SEQ ID NOs.: 8-3795, a gene product encoded by a nucleic acid having at least 70% nucleotide sequence identity as determined using BLASTN version 2.0 with the default parameters to a nucleic acid encoding a gene product whose expression is inhibited by an antisense nucleic acid comprising a nucleotide sequence selected from the group consisting of SEQ ID NOs:8-3795, a gene product having at least 25% amino acid identity as determined using FASTA version 3.0t78 with the default parameters to a gene product whose expression is inhibited by an antisense nucleic acid comprising a nucleotide sequence selected from the group consisting of SEQ ID NOs.: 8-3795, a gene product encoded by a nucleic acid comprising a nucleotide sequence which hybridizes to a nucleic acid selected from the group consisting of SEQ ID NOs.: 8-3795 under stringent conditions, a gene product encoded by a nucleic acid comprising a nucleotide sequence which hybridizes to a nucleic acid selected from the group consisting of SEQ ID NOs.: 8-3795 under moderate conditions, and a gene product whose activity may be complemented by the gene product whose activity is inhibited by a nucleic acid selected from the group consisting of SEQ ID NOs: 8-3795.

255. The method of Paragraph 254, wherein said antisense nucleic acid comprises a nucleotide sequence having at least 70% nucleotide sequence identity as determined using BLASTN version 2.0 with the default parameters to a nucleotide seqence selected from the group consisting of SEQ ID NOs.: 8-3795, a proliferation inhibiting portion thereof, a nucleic acid comprising a nucleotide sequence which hybridizes to a nucleic acid selected from the group consisting of SEQ ID NOs.: 8-3795 under stringent conditions, and a nucleic acid which comprising a nucleotide sequence which hybridizes to a nucleic acid selected from the group consisting of SEQ ID NOs.: 8-3795 under moderate conditions.

256. The method of Paragraph 254, wherein said cell is selected from the group consisting of Anaplasma marginale, Aspergillus fumigatus, Bacillus anthracis, Bacterioides fragilis Bordetella pertussis, Burkholderia cepacia, Campylobacter jejuni, Candida albicans, Candida glabrata (also called Torulopsis glabrata), Candida tropicalis, Candida parapsilosis, Candida guilliermondii, Candida krusei, Candida kefyr (also called Candida pseudotropicalis), Candida dubliniensis, Chlamydia pneumoniae, Chlamydia trachomatus, Clostridium botulinum, Clostridium difficile, Clostridium perfringens, Coccidiodes immitis, Corynebacterium diptheriae, Cryptococcus neoformans, Enterobacter cloacae, Enterococcus faecalis, Enterococcus faecium, Escherichia coli, Haemophilus influenzae, Helicobacter pylori, Histoplasma capsulatum, Klebsiella pneumoniae, Listeria monocytogenes, Mycobacterium leprae, Mycobacterium tuberculosis, Neisseria gonorrhoeae, Neisseria meningitidis, Nocardia asteroides, Pasteurella haemolytica, Pasteurella multocida, Pneumocystis carinii, Proteus vulgaris, Pseudomonas aeruginosa, Salmonella bongori, Salmonella cholerasuis, Salmonella enterica, Salmonella paratyphi, Salmonella typhi, Salmonella typhimurium, Staphylococcus aureus, Listeria monocytogenes, Moxarella catarrhalis, Shigella boydii, Shigella dysenteriae, Shigella flexneri, Shigella sonnet, Staphylococcus epidermidis, Streptococcus pneumoniae, Streptococcus mutans, Treponema pallidum, Yersinia enterocolitica, Yersinia pestis and any species falling within the genera of any of the above species.

257. The method of Paragraph 254, wherein said cell is not an E. coli cell.

258. The method of Paragraph 254, wherein said gene is from an organism other than E. coli.

259. The method of Paragraph 254, wherein said cell is contacted with said antisense nucleic acid by introducing a plasmid which transcribes said antisense nucleic acid into said cell population.

260. The method of Paragraph 254, wherein said cell is contacted with said antisense nucleic acid by introducing a phage which transcribes said antisense nucleic acid into said cell population.

261. The method of Paragraph 254, wherein said cell is contacted with said antisense nucleic acid by transcribing said antisense nucleic acid from the chromosome of cells in said cell population.

262. The method of Paragraph 254, wherein said cell is contacted with said antisense nucleic acid by introducing a promoter adjacent to a chromosomal copy of said antisense nucleic acid such that said promoter directs the synthesis of said antisense nucleic acid.

263. The method of Paragraph 254, wherein said cell is contacted with said antisense nucleic acid by introducing a retron which expresses said antisense nucleic acid into said cell population.

264. The method of Paragraph 254, wherein said cell is contacted with said antisense nucleic acid by introducing a ribozyme into said cell-population, wherein a binding portion of said ribozyme is complementary to said antisense oligonucleotide.

265. The method of Paragraph 254, wherein said cell is contacted with said antisense nucleic acid by introducing a liposome comprising said antisense oligonucleotide into said cell.

266. The method of Paragraph 254, wherein said cell is contacted with said antisense nucleic acid by electroporation of said antisense nucleic acid into said cell.

267. The method of Paragraph 254, wherein said antisense nucleic acid has at least 70% nucleotide sequence identity as determined using BLASTN version 2.0 with the default parameters to a nucleotide sequence comprising at least 10, at least 20, at least 25, at least 30, at least 50 or more than 50 consecutive nucleotides of one of SEQ ID NOs.: 8-3795.

268. The method of Paragraph 254 wherein said antisense nucleic acid is a synthetic oligonucleotide.

269. The method of Paragraph 254, wherein said gene comprises a nucleic acid selected from the group consisting of a nucleic acid comprising a nucleic acid having at least 70% nucleotide sequence identity as determined using BLASTN version 2.0 with the default parameters to a nucleotide sequence selected from the group consisting of SEQ ID NOS.: 3796-3800, 3806-4860, 5916-10012, a nucleic acid -comprising a nucleotide sequence which hybridizes to a sequence selected from the group consisting of SEQ ID NOS.: 3796-3800, 3806-4860, 5916-10012 under stringent conditions, and a nucleic acid comprising a nucleotide sequence which hybridizes to a nucleotide sequence selected from the group consisting of SEQ ID NOS.: 3796-3800, 3806-4860, 5916-10012 under moderate condtions.

270. A method for identifying a gene which is required for proliferation of a cell comprising:

(a) contacting a cell with an antisense nucleic acid selected from the group consisting of a nucleic acid at least 70% nucleotide sequence identity as determined using BLASTN version 2.0 with the default parameters to a nucleotide sequence selected from the group consisting of SEQ ID NOs.: 8-3795 or a proliferation-inhibiting portion thereof, a nucleic acid comprising a nucleotide sequence which hybridizes to a nucleic acid selected from the group consisting of SEQ ID NOs.: 8-3795 under stringent conditions, and a nucleic acid comprising a nucleotide sequence which hybridizes to a nucleic acid selected from the group consisting of SEQ ID NOs.: 8-3795 under moderate conditions, wherein said cell is a cell other than the organism from which said nucleic acid was obtained;

(b) determining whether said nucleic acid inhibits proliferation of said cell; and

(c) identifying the gene in said cell which encodes the mRNA which is complementary to said antisense nucleic acid or a portion thereof.

271. The method of Paragraph 270, wherein said cell is selected from the group consisting of Staphylococcus species, Streptococcus species, Enterococcus species, Mycobacterium species, Clostridium species, and Bacillus species.

272. The method of Paragraph 270 wherein said cell is selected from the group consisting of Anaplasma marginale, Aspergillus fumigatus, Bacillus anthracis, Bacterioides fragilis Bordetella pertussis, Burkholderia cepacia, Campylobacter jejuni, Candida albicans, Candida glabrata (also called Torulopsis glabrata), Candida tropicalis, Candida parapsilosis, Candida guilliermondii, Candida krusei, Candida kefyr (also called Candida pseudotropicalis), Candida dubliniensis, Chlamydia pneumoniae, Chlamydia trachomatus, Clostridium botulinum, Clostridium difficile, Clostridium perfringens, Coccidiodes immitis, Corynebacterium diptheriae, Cryptococcus neoformans, Enterobacter cloacae, Enterococcus faecalis, Enterococcus faecium, Escherichia coli, Haemophilus influenzae, Helicobacter pylori, Histoplasma capsulatum, Klebsiella pneumoniae, Listeria monocytogenes, Mycobacterium leprae, Mycobacterium tuberculosis, Neisseria gonorrhoeae, Neisseria meningitidis, Nocardia asteroides, Pasteurella haemolytica, Pasteurella multocida, Pneumocystis carinii, Proteus vulgaris, Pseudomonas aeruginosa, Salmonella bongori, Salmonella cholerasuis, Salmonella enterica, Salmonella paratyphi, Salmonella typhi, Salmonella typhimurium, Staphylococcus aureus, Listeria monocytogenes, Moxarella catarrhalis, Shigella boydii, Shigella dysenteriae, Shigella flexneri, Shigella sonnei, Staphylococcus epidermidis, Streptococcus pneumoniae, Streptococcus mutans, Treponema pallidum, Yersinia enterocolitica, Yersinia pestis and any species falling within the genera of any of the above species.

273. The method of Paragraph 270, wherein said cell is not E. coli.

274. The method of Paragraph 270, further comprising operably linking said antisense nucleic acid to a promoter which is functional in said cell, said promoter being included in a vector, and introducing said vector into said cell.

275. A method for identifying a compound having the ability to inhibit proliferation of a cell comprising:

(a) identifying a homolog of a gene or gene product whose activity or level is inhibited by an antisense nucleic acid in a test cell, wherein said test cell is not the microorgaism from which the antisense nucleic acid was obtained, wherein said antisense nucleic acid is selected from the group consisting of a nucleic acid having at least 70% nucleotide sequence identity as determined using BLASTN version 2.0 with the default parameters to a nucleotide sequence selected from the group consisting of SEQ ID NOs. 8-3795, a nucleic acid comprising a nucleotide sequence which hybridizes to a nucleic acid selected from the group consisting of SEQ ID NOs.: 8-3795 under stringent conditions, and a nucleic acid comprising a nucleotide sequence which hybridizes to a nucleic acid selected from the group consisting of SEQ ID NOs.: 8-3795 under moderate conditions;

(b) identifying an inhibitory nucleic acid sequence which inhibits the activity of said homolog in said test cell;

(c) contacting said test cell with a sublethal level of said inhibitory nucleic acid, thus sensitizing said cell;

(d) contacting the sensitized cell of step (c) with a compound; and

(e) determining the degree to which said compound inhibits proliferation of said sensitized cell relative to a cell which does not express said inhibitory nucleic acid.

276. The method of Paragraph 275, wherein said determining step comprises determining whether said compound inhibits proliferation of said sensitized test cell to a greater extent than said compound inhibits proliferation of a nonsensitized test cell.

277. The method of Paragraph 275, wherein step (a) comprises identifying a homologous nucleic acid to a gene or gene product whose activity or level is inhibited by a nucleic acid having at least 70% nucleotide sequence identity as determined using BLASTN version 2.0 with the default parameters to a nucleotide sequence selected from the group consisting of SEQ ID NOs. 8-3795 or a nucleic acid encoding a homologous polypeptide to a polypeptide whose activity or level is inhibited by a nucleic acid having at least 70% nucleotide sequence identity as determined using BLASTN version 2.0 with the default parameters to a nucleotide sequence selected from the group consisting of SEQ ID NOs. 8-3795 by using an algorithm selected from the group consisting of BLASTN version 2.0 with the default parameters and FASTA version 3.0t78 algorithm with the default parameters to identify said homologous nucleic acid or said nucleic acid encoding a homologous polypeptide in a database.

278. The method of Paragraph 275 wherein said step (a) comprises identifying a homologous nucleic acid or a nucleic acid encoding a homologous polypeptide by identifying nucleic acids comprising nucleotide sequences which hybridize to said nucleic acid having at least 70% nucleotide sequence identity as determined using BLASTN version 2.0 with the default parameters to a nucleotide sequence selected from the group consisting of SEQ ID NOs. 8-3795 or the complement of the nucleotide sequence of said nucleic acid selected from the group consisting of SEQ ID NOs. 8-3795.

279. The method of Paragraph 275 wherein step (a) comprises expressing a nucleic acid having at least 70% nucleic acid identity as determined using BLASTN version 2.0 with the default parameters to a sequence selected from the group consisting of SEQ ID NOs. 8-3795 in said test cell.

280. The method of Paragraph 275, wherein step (a) comprises identifying a homologous nucleic acid or a nucleic acid encoding a homologous polypeptide in an test cell selected from the group consisting of Anaplasma marginale, Aspergillus fumigatus, Bacillus anthracis, Bacterioides fragilis Bordetella pertussis, Burkholderia cepacia, Campylobacter jejuni, Candida albicans, Candida glabrata (also called Torulopsis glabrata), Candida tropicalis, Candida parapsilosis, Candida guilliermondii, Candida krusei, Candida kefyr (also called Candida pseudotropicalis), Candida dubliniensis, Chlamydia pneumoniae, Chlamydia trachomatus, Clostridium botulinum, Clostridium difficile, Clostridium perfringens, Coccidiodes immitis, Corynebacterium diptheriae, Cryptococcus neoformans, Enterobacter cloacae, Enterococcus faecalis, Enterococcus faecium, Escherichia coli, Haemophilus influenzae, Helicobacter pylori, Histoplasma capsulatum, Klebsiella pneumoniae, Listeria monocytogenes, Mycobacterium leprae, Mycobacterium tuberculosis, Neisseria gonorrhoeae, Neisseria meningitidis, Nocardia asteroides, Pasteurella haemolytica, Pasteurella multocida, Pneumocystis carinii, Proteus vulgaris, Pseudomonas aeruginosa, Salmonella bongori, Salmonella cholerasuis, Salmonella enterica, Salmonella paratyphi, Salmonella typhi, Salmonella typhimurium, Staphylococcus aureus, Listeria monocytogenes, Moxarella catarrhalis, Shigella boydii, Shigella dysenteriae, Shigella flexneri, Shigella sonnei, Staphylococcus epidermidis, Streptococcus pneumoniae, Streptococcus mutans, Treponema pallidum, Yersinia enterocolitica, Yersinia pestis and any species falling within the genera of any of the above species.

281. The method of Paragraph 275, wherein step (a) comprises identifying a homologous nucleic acid or a nucleic acid encoding a homologous polypeptide in a test cell other than E. coli.

282. The method of Paragraph 275, wherein said inhibitory nucleic acid is an antisense nucleic acid.

283. The method of Paragraph 275, wherein said inhibitory nucleic acid comprises an antisense nucleic acid to a portion of said homolog.

284. The method of Paragraph 275, wherein said inhibitory nucleic acid comprises an antisense nucleic acid to a portion of the operon encoding said homolog.

285. The method of Paragraph 275, wherein the step of contacting the cell with a sublethal level of said inhibitory nucleic acid comprises directly contacting said cell with said inhibitory nucleic acid.

286. The method of Paragraph 275, wherein the step of contacting the cell with a sublethal level of said inhibitory nucleic acid comprises expressing an antisense nucleic acid to said homolog in said cell.

287. The method of Paragraph 275, wherein said gene product comprises a polypeptide comprising an amino acid sequence selected from the group consisting of SEQ ID NOs.: 3801-3805, 4861-5915, 10013-14110.

288. The method of Paragraph 275, wherein said gene comprises a nucleic acid selected from the group consisting of a nucleic acid comprising a nucleic acid having at least 70% nucleotide sequence identity as determined using BLASTN version 2.0 with the default parameters to a sequence selected from the group consisting of SEQ ID NOS.: 3796-3800, 3806-4860, 5916-10012, a nucleic acid comprising a nucleotide sequence which hybridizes to a nucleotide sequence selected from the group consisting of SEQ ID NOS.: 3796-3800, 3806-4860, 5916-10012 under stringent conditions, and a nucleic acid comprising a nucleotide sequence which hybridizes to a nucleotide sequence selected from the group consisting of SEQ ID NOS.: 3796-3800, 3806-4860, 5916-10012 under moderate condtions.

289. A compound identified using the method of Paragraph 275.

290. A method of identifying a compound having the ability to inhibit proliferation comprising:

(a) sensitizing a test cell by contacting said test cell with a sublethal level of an antisense nucleic acid, wherein said antisense nucleic acid is selected from the group consisting of a nucleic acid having at least 70% nucleotide sequence identity as determined using BLASTN version 2.0 with the default parameters to a nucleotide sequence selected from the group consisting of SEQ ID NOs. 8-3795 or a portion thereof which inhibits the proliferation of the cell from which said nucleic acid was obtained, a nucleic acid comprising a nucleotide sequence which hybridizes to a nucleic acid selected from the group consisting of SEQ ID NOs.: 8-3795 under stringent conditions, and a nucleic acid comprising a nucleotide sequence which hybridizes to a nucleic acid selected from the group consisting of SEQ ID NOs.: 8-3795 under moderate conditionst;

(b) contacting the sensitized test cell of step (a) with a compound; and

(c) determining the degree to which said compound inhibits proliferation of said sensitized test cell relative to a cell which does not contain said antisense nucleic acid.

291. The method of Paragraph 290, wherein said determining step comprises determining whether said compound inhibits proliferation of said sensitized test cell to a greater extent than said compound inhibits proliferation of a nonsensitized test cell.

292. A compound identified using the method of Paragraph 290.

293. The method of Paragraph 290, wherein said test cell is selected from the group consisting of Anaplasma marginale, Aspergillus fumigatus, Bacillus anthracis, Bacterioides fragilis Bordetella pertussis, Burkholderia cepacia, Campylobacter jejuni, Candida albicans, Candida glabrata (also called Torulopsis glabrata), Candida tropicalis, Candida parapsilosis, Candida guilliermondii, Candida krusei, Candida kefyr (also called Candida pseudotropicalis), Candida dubliniensis, Chlamydia pneumoniae, Chlamydia trachomatus, Clostridium botulinum, Clostridium difficile, Clostridium perfringens, Coccidiodes immitis, Corynebacterium diptheriae, Cryptococcus neoformans, Enterobacter cloacae, Enterococcus faecalis, Enterococcus faecium, Escherichia coli, Haemophilus influenzae, Helicobacter pylori, Histoplasma capsulatum, Klebsiella pneumoniae, Listeria monocytogenes, Mycobacterium leprae, Mycobacterium tuberculosis, Neisseria gonorrhoeae, Neisseria meningitidis, Nocardia asteroides, Pasteurella haemolytica, Pasteurella multocida, Pneumocystis carinii, Proteus vulgaris, Pseudomonas aeruginosa, Salmonella bongori, Salmonella cholerasuis, Salmonella enterica, Salmonella paratyphi, Salmonella typhi, Salmonella typhimurium, Staphylococcus aureus, Listeria monocytogenes, Moxarella catarrhalis, Shigella boydii, Shigella dysenteriae, Shigella flexneri, Shigella sonnei, Staphylococcus epidermidis, Streptococcus pneumoniae, Streptococcus mutans, Treponema pallidum, Yersinia enterocolitica, Yersinia pestis and any species falling within the genera of any of the above species.

294. The method of Paragraph 290, wherein the test cell is not E. coli.

295. A method for identifying a compound having activity against a biological pathway required for proliferation comprising:

(a) sensitizing a cell by providing a sublethal level of an antisense nucleic acid complementary to a nucleic acid encoding a gene product required for proliferation, wherein said gene product is selected from the group consisting of a gene product having at least 70% nucleotide sequence identity as determined using BLASTN version 2.0 with the default parameters to a gene product whose expression is inhibited by an antisense nucleic acid comprising a nucleotide sequence selected from the group consisting of SEQ ID NOs.: 8-3795, a gene product encoded by a nucleic acid having at least 70% nucleotide sequence identity as determined using BLASTN version 2.0 with the default parameters to a nucleic acid encoding a gene product whose expression is inhibited by an antisense nucleic acid comprising a nucleotide sequence selected from the group consisting of SEQ ID NOs:8-3795, a gene product having at least 25% amino acid identity as determined using FASTA version 3.0t78 with the default parameters to a gene product whose expression is inhibited by an antisense nucleic acid comprising a nucleotide sequence selected from the group consisting of SEQ ID NOs.: 8-3795, a gene product encoded by a nucleic acid comprising a nucleotide sequence which hybridizes to a nucleic acid selected from the group consisting of SEQ ID NOs.: 8-3795 under stringent conditions, a gene product encoded by a nucleic acid comprising a nucleotide sequence which hybridizes to a nucleic acid selected from the group consisting of SEQ ID NOs.: 8-3795 under moderate conditions, and a gene product whose activity may be complemented by the gene product whose activity is inhibited by a nucleic acid selected from the group consisting of SEQ ID NOs: 8-3795;

(b) contacting the sensitized cell with a compound; and

(c) determining the extent to which said compound inhibits the growth of said sensitized cell relative to a cell which does not contain said antisense nucleic acid.

296. The method of Paragraph 295, wherein said determining step comprises determining whether said compound inhibits the growth of said sensitized cell to a greater extent than said compound inhibits the growth of a nonsensitized cell.

297. The method of Paragraph 295, wherein said cell is selected from the group consisting of bacterial cells, fungal cells, plant cells, and animal cells.

298. The method of Paragraph 295, wherein said cell is a Gram positive bacterium.

299. The method of Paragraph 298, wherein said Gram positive bacterium is selected from the group consisting of Staphylococcus species, Streptococcus species, Enterococcus species, Mycobacterium species, Clostridium species, and Bacillus species.

300. The method of Paragraph 299, wherein said Gram positive bacterium is Staphylococcus aureus.

301. The method of Paragraph 298, wherein said Gram positive bacterium is selected from the group consisting of Staphylococcus aureus RN450 and Staphylococcus aureus RN4220.

302. The method of Paragraph 295, wherein said cell is selected from the group consisting of Anaplasma marginale, Aspergillus fumigatus, Bacillus anthracis, Bacterioides fragilis Bordetella pertussis, Burkholderia cepacia, Campylobacter jejuni, Candida albicans, Candida glabrata (also called Torulopsis glabrata), Candida tropicalis, Candida parapsilosis, Candida guilliermondii, Candida krusei, Candida kefyr (also called Candida pseudotropicalis), Candida dubliniensis, Chlamydia pneumoniae, Chlamydia trachomatus, Clostridium botulinum, Clostridium difficile, Clostridium perfringens, Coccidiodes immitis, Corynebacterium diptheriae, Cryptococcus neoformans, Enterobacter cloacae, Enterococcus faecalis, Enterococcus faecium, Escherichia coli, Haemophilus influenzae, Helicobacter pylori, Histoplasma capsulatum, Klebsiella pneumoniae, Listeria monocytogenes, Mycobacterium leprae, Mycobacterium tuberculosis, Neisseria gonorrhoeae, Neisseria meningitidis, Nocardia asteroides, Pasteurella haemolytica, Pasteurella multocida, Pneumocystis carinii, Proteus vulgaris, Pseudomonas aeruginosa, Salmonella bongori, Salmonella cholerasuis, Salmonella enterica, Salmonella paratyphi, Salmonella typhi, Salmonella typhimurium, Staphylococcus aureus, Listeria monocytogenes, Moxarella catarrhalis, Shigella boydii, Shigella dysenteriae, Shigella flexneri, Shigella sonnei, Staphylococcus epidermidis, Streptococcus pneumoniae, Streptococcus mutans, Treponema pallidum, Yersinia enterocolitica, Yersinia pestis and any species falling within the genera of any of the above species.

303. The method of Paragraph 295, wherein said cell is not an E. coli cell.

304. The method of Paragraph 295, wherein said gene product is from an organism other than E. coli.

305. The method of Paragraph 295, wherein said antisense nucleic acid is transcribed from an inducible promoter.

306. The method of Paragraph 305, further comprising contacting the cell with an agent which induces expression of said antisense nucleic acid from said inducible promoter, wherein said antisense nucleic acid is expressed at a sublethal level.

307. The method of Paragraph 295, wherein inhibition of proliferation is measured by monitoring the optical density of a liquid culture.

308. The method of Paragraph 295, wherein said gene product comprises a polypeptide having at least 25% amino acid identity as determined using FASTA version 3.0t78 with the default parameters to a sequence selected from the group consisting of SEQ ID NOs.: 3801-3805, 4861-5915, 10013-14110.

309. The method of Paragraph 295, wherein said nucleic acid encoding said gene product comprises a nucleic acid selected from the group consisting of a nucleic acid comprising a nucleic acid having at least 70% nucleotide sequence identity as determined using BLASTN version 2.0 with the default parameters to a nucleotide sequence selected from the group consisting of SEQ ID NOS.: 3796-3800, 3806-4860, 5916-10012, a nucleic acid comprising a nucleotide sequence which hybridizes to a nucleotide sequence selected from the group consisting of SEQ ID NOS.: 3796-3800, 3806-4860, 5916-10012 under stringent conditions, and a nucleic acid comprising a nucleotide sequence which hybridizes to a nucleotide sequence selected from the group consisting of SEQ ID NOS.: 3796-3800, 3806-4860, 5916-10012 under moderate condtions.

310. A compound identified using the method of Paragraph 295.

311. A method for identifying a compound having the ability to inhibit cellular proliferation comprising:

(a) contacting a cell with an agent which reduces the activity or level of a gene product required for proliferation of said cell, wherein said gene product is selected from the group consisting of a gene product having at least 70% nucleotide sequence identity as determined using BLASTN version 2.0 with the default parameters to a gene product whose expression is inhibited by an antisense nucleic acid comprising a nucleotide sequence selected from the group consisting of SEQ ID NOs.: 8-3795, a gene product encoded by a nucleic acid having at least 70% nucleotide sequence identity as determined using BLASTN version 2.0 with the default parameters to a nucleic acid encoding a gene product whose expression is inhibited by an antisense nucleic acid comprising a nucleotide sequence selected from the group consisting of SEQ ID NOs:8-3795, a gene product having at least 25% amino acid identity as determined using FASTA version 3.0t78 with the default parameters to a gene product whose expression is inhibited by an antisense nucleic acid comprising a nucleotide sequence selected from the group consisting of SEQ ID NOs.: 8-3795, a gene product encoded by a nucleic acid comprising a nucleotide sequence which hybridizes to a nucleic acid selected from the group consisting of SEQ ID NOs.: 8-3795 under stringent conditions, a gene product encoded by a nucleic acid comprising a nucleotide sequence which hybridizes to a nucleic acid selected from the group consisting of SEQ ID NOs.: 8-3795 under moderate conditions, and a gene product whose activity may be complemented by the gene product whose activity is inhibited by a nucleic acid selected from the group consisting of SEQ ID NOs: 8-3795;

(b) contacting said cell with a compound; and

(c) determining the degree to which said compound reduces proliferation of said contacted cell relative to a cell which was not contacted with said agent.

312. The method of Paragraph 311, wherein said determining step comprises determining whether said compound reduces proliferation of said contacted cell to a greater extent than said compound reduces proliferation of cells which have not been contacted with said agent.

313. The method of Paragraph 311, wherein said cell is selected from the group consisting of Anaplasma marginale, Aspergillus fumigatus, Bacillus anthracis, Bacterioides fragilis Bordetella pertussis, Burkholderia cepacia, Campylobacter jejuni, Candida albicans, Candida glabrata (also called Torulopsis glabrata), Candida tropicalis, Candida parapsilosis, Candida guilliermondii, Candida krusei, Candida kefyr (also called Candida pseudotropicalis), Candida dubliniensis, Chlamydia pneumoniae, Chlamydia trachomatus, Clostridium botulinum, Clostridium difficile, Clostridium perfringens, Coccidiodes immitis, Corynebacterium diptheriae, Cryptococcus neoformans, Enterobacter cloacae, Enterococcus faecalis, Enterococcus faecium, Escherichia coli, Haemophilus influenzae, Helicobacter pylori, Histoplasma capsulatum, Klebsiella pneumoniae, Listeria monocytogenes, Mycobacterium leprae, Mycobacterium tuberculosis, Neisseria gonorrhoeae, Neisseria meningitidis, Nocardia asteroides, Pasteurella haemolytica, Pasteurella multocida, Pneumocystis carinii, Proteus vulgaris, Pseudomonas aeruginosa, Salmonella bongori, Salmonella cholerasuis, Salmonella enterica, Salmonella paratyphi, Salmonella typhi, Salmonella typhimurium, Staphylococcus aureus, Listeria monocytogenes, Moxarella catarrhalis, Shigella boydii, Shigella dysenteriae, Shigella flexneri, Shigella sonnei, Staphylococcus epidermidis, Streptococcus pneumoniae, Streptococcus mutans, Treponema pallidum, Yersinia enterocolitica, Yersinia pestis and any species falling within the genera of any of the above species.

314. The method of Paragraph 311, wherein said cell is not an E. coli cell.

315. The method of Paragraph 311, wherein said gene product is from an organism other than E. coli.

316. The method of Paragraph 311, wherein said agent which reduces the activity or level of a gene product required for proliferation of said cell comprises an antisense nucleic acid to a gene or operon required for proliferation.

317. The method of Paragraph 311, wherein said agent which reduces the activity or level of a gene product required for proliferation of said cell comprises a compound known to inhibit growth or proliferation of a cell.

318. The method of Paragraph 311, wherein said cell contains a mutation which reduces the activity or level of said gene product required for proliferation of said cell.

319. The method of Paragraph 311, wherein said mutation is a temperature sensitive mutation.

320. The method of Paragraph 311, wherein said gene product comprises a gene product comprises a polypeptide having at least 25% amino acid identity as determined using FASTA version 3.0t78 with the default parameters to an amino acid sequence selected from the group consisting of SEQ ID NOs.: 3801-3805, 4861-5915, 10013-14110.

321. A compound identified using the method of Paragraph 311.

322. A method for identifying the biological pathway in which a proliferation-required gene product or a gene encoding a proliferation-required gene product lies comprising:

(a) providing a sublethal level of an antisense nucleic acid which inhibits the activity or reduces the level of said gene encoding a proliferation-required gene product or said said proliferation-required gene product in a test cell, wherein said proliferation-required gene product is selected from the group consisting of a gene product having at least 70% nucleotide sequence identity as determined using BLASTN version 2.0 with the default parameters to a gene product whose expression is inhibited by an antisense nucleic acid comprising a nucleotide sequence selected from the group consisting of SEQ ID NOs.: 8-3795, a gene product encoded by a nucleic acid having at least 70% nucleotide sequence identity as determined using BLASTN version 2.0 with the default parameters to a nucleic acid encoding a gene product whose expression is inhibited by an antisense nucleic acid comprising a nucleotide sequence selected from the group consisting of SEQ ID NOs:8-3795, a gene product having at least 25% amino acid identity as determined using FASTA version 3.0t78 with the default parameters to a gene product whose expression is inhibited by an antisense nucleic acid comprising a nucleotide sequence selected from the group consisting of SEQ ID NOs.: 8-3795, a gene product encoded by a nucleic acid comprising a nucleotide sequence which hybridizes to a nucleic acid selected from the group consisting of SEQ ID NOs.: 8-3795 under stringent conditions, a gene product encoded by a nucleic acid comprising a nucleotide sequence which hybridizes to a nucleic acid selected from the group consisting of SEQ ID NOs.: 8-3795 under moderate conditions, and a gene product whose activity may be complemented by the gene product whose activity is inhibited by a nucleic acid selected from the group consisting of SEQ ID NOs: 8-3795;

(b) contacting said test cell with a compound known to inhibit growth or proliferation of a cell, wherein the biological pathway on which said compound acts is known; and

(c) determining the degree to which said compound inhibits proliferation of said test cell relative to a cell which does not contain said antisense nucleic acid.

323. The method of Paragraph 322, wherein said determining step comprises determining whether said test cell has a substantially greater sensitivity to said compound than a cell which does not express said sublethal level of said antisense nucleic acid.

324. The method of Paragraph 322, wherein said gene product comprises a polypeptide having at least 25% amino acid identity as determined using FASTA version 3.0t78 with the default parameters to an amino acid sequence selected from the group consisting of SEQ ID NOs.: 3801-3805, 4861-5915, 10013-14110.

325. The method of Paragraph 322, wherein said test cell is selected from the group consisting of Anaplasma marginale, Aspergillus fumigatus, Bacillus anthracis, Bacterioides fragilis Bordetella pertussis, Burkholderia cepacia, Campylobacter jejuni, Candida albicans, Candida glabrata (also called Torulopsis glabrata), Candida tropicalis, Candida parapsilosis, Candida guilliermondii, Candida krusei, Candida kefyr (also called Candida pseudotropicalis), Candida dubliniensis, Chlamydia pneumoniae, Chlamydia trachomatus, Clostridium botulinum, Clostridium difficile, Clostridium perfringens, Coccidiodes immitis, Corynebacterium diptheriae, Cryptococcus neoformans, Enterobacter cloacae, Enterococcus faecalis, Enterococcus faecium, Escherichia coli, Haemophilus influenzae, Helicobacter pylori, Histoplasma capsulatum, Klebsiella pneumoniae, Listeria monocytogenes, Mycobacterium leprae, Mycobacterium tuberculosis, Neisseria gonorrhoeae, Neisseria meningitidis, Nocardia asteroides, Pasteurella haemolytica, Pasteurella multocida, Pneumocystis carinii, Proteus vulgaris, Pseudomonas aeruginosa, Salmonella bongori, Salmonella cholerasuis, Salmonella enterica, Salmonella paratyphi, Salmonella typhi, Salmonella typhimurium, Staphylococcus aureus, Listeria monocytogenes, Moxarella catarrhalis, Shigella boydii, Shigella dysenteriae, Shigella flexneri, Shigella sonnei, Staphylococcus epidermidis, Streptococcus pneumoniae, Streptococcus mutans, Treponema pallidum, Yersinia enterocolitica, Yersinia pestis and any species falling within the genera of any of the above species.

326. The method of Paragraph 322, wherein said test cell is not an E. coli cell.

327. The method of Paragraph 322, wherein said gene product is from an organism other than E. coli.

328. A method for determining the biological pathway on which a test compound acts comprising:

(a) providing a sublethal level of an antisense nucleic acid complementary to a proliferation-required nucleic acid in a cell, thereby producing a sensitized cell, wherein said antisense nucleic acid is selected from the group consisting of a nucleic acid having at least 70% nucleotide sequence identity as determined using BLASTN version 2.0 with the default parameters to a nucleotide sequence selected from the group consisting of SEQ ID NOs:8-3795 or a proliferation-inhibiting portion thereof a nucleic acid comprising a nucleotide sequence which hybridizes to a nucleic acid selected from the group consisting of SEQ ID NOs.: 8-3795 under stringent conditions, and a nucleic acid comprising a nucleotide sequence which hybridizes to a nucleic acid selected from the group consisting of SEQ ID NOs.: 8-3795 under moderate conditions and wherein the biological pathway in which said proliferation-required nucleic acid or a protein encoded by said proliferation-required polypeptide lies is known,

(b) contacting said cell with said test compound; and

(c) determining the degree to which said compound inhibits proliferation of said sensitized cell relative to a cell which does not contain said antisense nucleic acid.

329. The method of Paragraph 328, wherein said determining step comprises determining whether said sensitized cell has a substantially greater sensitivity to said test compound than a cell which does not express said sublethal level of said antisense nucleic acid.

330. The method of Paragraph 328, further comprising:

(d) providing a sublethal level of a second antisense nucleic acid complementary to a second proliferation-required nucleic acid in a second cell, wherein said second proliferation-required nucleic acid is in a different biological pathway than said proliferation-required nucleic acid in step (a); and

(e) determining whether said second cell does not have a substantially greater sensitivity to said test compound than a cell which does not express said sublethal level of said second antisense nucleic acid, wherein said test compound is specific for the biological pathway against which the antisense nucleic acid of step (a) acts if said sensitized cell has substantially greater sensitivity to said test compound than said second cell.

331. The method of Paragraph 328, wherein said sensitized cell is selected from the group consisting of Anaplasma marginale, Aspergillus fumigatus, Bacillus anthracis, Bacterioides fragilis Bordetella pertussis, Burkholderia cepacia, Campylobacter jejuni, Candida albicans, Candida glabrata (also called Torulopsis glabrata), Candida tropicalis, Candida parapsilosis, Candida guilliermondii, Candida krusei, Candida kefyr (also called Candida pseudotropicalis), Candida dubliniensis, Chlamydia pneumoniae, Chlamydia trachomatus, Clostridium botulinum, Clostridium difficile, Clostridium perfringens, Coccidiodes immitis, Corynebacterium diptheriae, Cryptococcus neoformans, Enterobacter cloacae, Enterococcus faecalis, Enterococcus faecium, Escherichia coli, Haemophilus influenzae, Helicobacter pylori, Histoplasma capsulatum, Klebsiella pneumoniae, Listeria monocytogenes, Mycobacterium leprae, Mycobacterium tuberculosis, Neisseria gonorrhoeae, Neisseria meningitidis, Nocardia asteroides, Pasteurella haemolytica, Pasteurella multocida, Pneumocystis carinii, Proteus vulgaris, Pseudomonas aeruginosa, Salmonella bongori, Salmonella cholerasuis, Salmonella enterica, Salmonella paratyphi, Salmonella typhi, Salmonella typhimurium, Staphylococcus aureus, Listeria monocytogenes, Moxarella catarrhalis, Shigella boydii, Shigella dysenteriae, Shigella flexneri, Shigella sonnei, Staphylococcus epidermidis, Streptococcus pneumoniae, Streptococcus mutans, Treponema pallidum, Yersinia enterocolitica, Yersinia pestis and any species falling within the genera of any of the above species.

332. The method of Paragraph 328, wherein said sensitized cell is not an E. coli cell.

333. The method of Paragraph 328, wherein said proliferation-required nucleic acid is from an organism other than E. coli.

334. A compound which inhibits proliferation by interacting with a gene encoding a gene product required for proliferation or with a gene product required for proliferation, wherein said gene product is selected from the group consisting of a gene product having at least 70% nucleotide sequence identity as determined using BLASTN version 2.0 with the default parameters to a gene product whose expression is inhibited by an antisense nucleic acid comprising a nucleotide sequence selected from the group consisting of SEQ ID NOs.: 8-3795, a gene product encoded by a nucleic acid having at least 70% nucleotide sequence identity as determined using BLASTN version 2.0 with the default parameters to a nucleic acid encoding a gene product whose expression is inhibited by an antisense nucleic acid comprising a nucleotide sequence selected from the group consisting of SEQ ID NOs:8-3795, a gene product having at least 25% amino acid identity as determined using FASTA version 3.0t78 with the default parameters to a gene product whose expression is inhibited by an antisense nucleic acid comprising a nucleotide sequence selected from the group consisting of SEQ ID NOs.: 8-3795, a gene product encoded by a nucleic acid comprising a nucleotide sequence which hybridizes to a nucleic acid selected from the group consisting of SEQ ID NOs.: 8-3795 under stringent conditions, a gene product encoded by a nucleic acid comprising a nucleotide sequence which hybridizes to a nucleic acid selected from the group consisting of SEQ ID NOs.: 8-3795 under moderate conditions, and a gene product whose activity may be complemented by the gene product whose activity is inhibited by a nucleic acid selected from the group consisting of SEQ ID NOs: 8-3795.

335. The compound of Paragraph 334, wherein said gene product comprises a polypeptide having at least 25% amino acid identity as determined using FASTA version 3.0t78 with the default parameters to a sequence selected from the group consisting of SEQ ID NOs.: 3801-3805, 4861-5915, 10013-14110.

336. The compound of Paragraph 334, wherein said gene comprises a nucleic acid selected from the group consisting of a nucleic acid comprising a nucleic acid having at least 70% nucleotide sequence identity as determined using BLASTN version 2.0 with the default parameters to a nucleotide sequence selected from the group consisting of SEQ ID NOS.: 3796-3800, 3806-4860, 5916-10012, a nucleic acid comprising a nucleotide sequence which hybridizes to a nucleotide sequence selected from the group consisting of SEQ ID NOS.: 3796-3800, 3806-4860, 5916-10012 under stringent conditions, and a nucleic acid comprising a nucleotide sequence which hybridizes to a nucleotide sequence selected from the group consisting of SEQ ID NOS.: 3796-3800, 3806-4860, 5916-10012 under moderate condtions.

337. A method for manufacturing an antibiotic comprising the steps of:

screening one or more candidate compounds to identify a compound that reduces the activity or level of a gene product required for proliferation wherein said gene product is selected from the group consisting of a gene product having at least 70% nucleotide sequence identity as determined using BLASTN version 2.0 with the default parameters to a gene product whose expression is inhibited by an antisense nucleic acid comprising a nucleotide sequence selected from the group consisting of SEQ ID NOs.: 8-3795, a gene product encoded by a nucleic acid having at least 70% nucleotide sequence identity as determined using BLASTN version 2.0 with the default parameters to a nucleic acid encoding a gene product whose expression is inhibited by an antisense nucleic acid comprising a nucleotide sequence selected from the group consisting of SEQ ID NOs:8-3795, a gene product having at least 25% amino acid identity as determined using FASTA version 3.0t78 with the default parameters to a gene product whose expression is inhibited by an antisense nucleic acid comprising a nucleotide sequence selected from the group consisting of SEQ ID NOs.: 8-3795, a gene product encoded by a nucleic acid comprising a nucleotide sequence which hybridizes to a nucleic acid selected from the group consisting of SEQ ID NOs.: 8-3795 under stringent conditions, a gene product encoded by a nucleic acid comprising a nucleotide sequence which hybridizes to a nucleic acid selected from the group consisting of SEQ ID NOs.: 8-3795 under moderate conditions, and a gene product whose activity may be complemented by the gene product whose activity is inhibited by a nucleic acid selected from the group consisting of SEQ ID NOs: 8-3795; and

manufacturing the compound so identified.

338. The method of Paragraph 337, wherein said screening step comprises performing any one of the methods of Paragraphs 205, 211, 222, 275, 290, 295, 311.

339. The method of Paragraph 337, wherein said gene product comprises a polypeptide having at least 25% amino acid identity as determined using FASTA version 3.0t78 with the default parameters to an amino acid sequence selected from the group consisting of SEQ ID NOs.: 3801-3805, 4861-5915, 10013-14110.

340. A method for inhibiting proliferation of a cell in a subject comprising administering an effective amount of a compound that reduces the activity or level of a gene product required for proliferation of said cell, wherein said gene product is selected from the group consisting of a gene product having at least 70% nucleotide sequence identity as determined using BLASTN version 2.0 with the default parameters to a gene product whose expression is inhibited by an antisense nucleic acid comprising a nucleotide sequence selected from the group consisting of SEQ ID NOs.: 8-3795, a gene product encoded by a nucleic acid having at least 70% nucleotide sequence identity as determined using BLASTN version 2.0 with the default parameters to a nucleic acid encoding a gene product whose expression is inhibited by an antisense nucleic acid comprising a nucleotide sequence selected from the group consisting of SEQ ID NOs:8-3795, a gene product having at least 25% amino acid identity as determined using FASTA version 3.0t78 with the default parameters to a gene product whose expression is inhibited by an antisense nucleic acid comprising a nucleotide sequence selected from the group consisting of SEQ ID NOs.: 8-3795, a gene product encoded by a nucleic acid comprising a nucleotide sequence which hybridizes to a nucleic acid selected from the group consisting of SEQ ID NOs.: 8-3795 under stringent conditions, a gene product encoded by a nucleic acid comprising a nucleotide sequence which hybridizes to a nucleic acid selected from the group consisting of SEQ ID NOs.: 8-3795 under moderate conditions, and a gene product whose activity may be complemented by the gene product whose activity is inhibited by a nucleic acid selected from the group consisting of SEQ ID NOs: 8-3795.

341. The method of Paragraph 340 wherein said subject is selected from the group consisting of vertebrates, mammals, avians, and human beings.

342. The method of Paragraph 340, wherein said gene product comprises a polypeptide having at least 25% amino acid identity as determined using FASTA version 3.0t78 with the default parameters to an amino acid sequence selected from the group consisting of SEQ ID NOs.: 3801-3805, 4861-5915, 10013-14110.

343. The method of Paragraph 340, wherein said cell is selected from the group consisting of Anaplasma marginale, Aspergillus fumigatus, Bacillus anthracis, Bacterioides fragilis Bordetella pertussis, Burkholderia cepacia, Campylobacter jejuni, Candida albicans, Candida glabrata (also called Torulopsis glabrata), Candida tropicalis, Candida parapsilosis, Candida guilliermondii, Candida krusei, Candida kefyr (also called Candida pseudotropicalis), Candida dubliniensis, Chlamydia pneumoniae, Chlamydia trachomatus, Clostridium botulinum, Clostridium difficile, Clostridium perfringens, Coccidiodes immitis, Corynebacterium diptheriae, Cryptococcus neoformans, Enterobacter cloacae, Enterococcus faecalis, Enterococcus faecium, Escherichia coli, Haemophilus influenzae, Helicobacter pylori, Histoplasma capsulatum, Klebsiella pneumoniae, Listeria monocytogenes, Mycobacterium leprae, Mycobacterium tuberculosis, Neisseria gonorrhoeae, Neisseria meningitidis, Nocardia asteroides, Pasteurella haemolytica, Pasteurella multocida, Pneumocystis carinii, Proteus vulgaris, Pseudomonas aeruginosa, Salmonella bongori, Salmonella cholerasuis, Salmonella enterica, Salmonella paratyphi, Salmonella typhi, Salmonella typhimurium, Staphylococcus aureus, Listeria monocytogenes, Moxarella catarrhalis, Shigella boydii, Shigella dysenteriae, Shigella flexneri, Shigella sonnei, Staphylococcus epidermidis, Streptococcus pneumoniae, Streptococcus mutans, Treponema pallidum, Yersinia enterocolitica, Yersinia pestis and any species falling within the genera of any of the above species.

344. The method of Paragraph 340, wherein said cell is not E. coli.

345. The method of Paragraph 340, wherein said gene product is from an organism other than E. coli.

### Definitions

By “biological pathway” is meant any discrete cell function or process that is carried out by a gene product or a subset of gene products. Biological pathways include anabolic, catabolic, enzymatic, biochemical and metabolic pathways as well as pathways involved in the production of cellular structures such as cell walls. Biological pathways that are usually required for proliferation of cells or microorganisms include, but are not limited to, cell division, DNA synthesis and replication, RNA synthesis (transcription), protein synthesis (translation), protein processing, protein transport, fatty acid biosynthesis, electron transport chains, cell wall synthesis, cell membrane production, synthesis and maintenance, and the like.

By “inhibit activity of a gene or gene product” is meant having the ability to interfere with the function of a gene or gene product in such a way as to decrease expression of the gene, in such a way as to reduce the level or activity of a product of .the gene or in such a way as to inhibit the interaction of the gene or gene product with other biological molecules required for its activity. Agents which inhibit the activity of a gene include agents that inhibit transcription of the gene, agents that inhibit processing of the transcript of the gene, agents that reduce the stability of the transcript of the gene, and agents that inhibit translation of the mRNA transcribed from the gene. In microorganisms, agents which inhibit the activity of a gene can act to decrease expression of the operon in which the gene resides or alter the folding or processing of operon RNA so as to reduce the level or activity of the gene product. The gene product can be a non-translated RNA such as ribosomal RNA, a translated RNA (mRNA) or the protein product resulting from translation of the gene mRNA. Of particular utility to the present invention are antisense RNAs that have activities against the operons or genes to which they specifically hybridze.

By “activity against a gene product” is meant having the ability to inhibit the function or to reduce the level or activity of the gene product in a cell. This includes, but is not limited to, inhibiting the enzymatic activity of the gene product or the ability of the gene product to interact with other biological molecules required for its activity, including inhibiting the gene product's assembly into a multimeric structure.

By “activity against a protein” is meant having the ability to inhibit the function or to reduce the level or activity of the protein in a cell. This includes, but is not limited to, inhibiting the enzymatic activity of the protein or the ability of the protein to interact with other biological molecules required for its activity, including inhibiting the protein's assembly into a multimeric structure.

By “activity against a nucleic acid” is meant having the ability to inhibit the function or to reduce the level or activity of the nucleic acid in a cell. This includes, but is not limited to, inhibiting the ability of the nucleic acid interact with other biological molecules required for its activity, including inhibiting the nucleic acid's assembly into a multimeric structure.

By “activity against a gene” is meant having the ability to inhibit the function or expression of the gene in a cell. This includes, but is not limited to, inhibiting the ability of the gene to interact with other biological molecules required for its activity.

By “activity against an operon” is meant having the ability to inhibit the function or reduce the level of one or more products of the operon in a cell. This includes, but is not limited to, inhibiting the enzymatic activity of one or more products of the operon or the ability of one or more products of the operon to interact with other biological molecules required for its activity.

By “antibiotic” is meant an agent which inhibits the proliferation of a cell or microorganism.

By “E. coli or Escherichia coli” is meant Escherichia coli or any organism previously categorized as a species of Shigella including Shigella boydii, Shigella flexneri, Shigella dysenteriae, Shigella sonnei, Shigella 2A.

By “homologous coding nucleic acid” is meant a nucleic acid homologous to a nucleic acid encoding a gene product whose activity or level is inhibited by a nucleic acid selected from the group consisting of SEQ ID NOs.: 8-3795 or a portion thereof. In some embodiments, the homologous coding nucleic acid may have at least 97%, at least 95%, at least 90%, at least 85%, at least 80%, or at least 70% nucleotide sequence identity to a nucleotide sequence selected from the group consisting of SEQ ID NOS.: 3796-3800, 3806-4860, 5916-10012 and fragments comprising at least 10, 15, 20, 25, 30, 35, 40, 50, 75, 100, 150, 200, 300, 400, or 500 consecutive nucleotides thereof. In other embodiments the homologous coding nucleic acids may have at least 97%, at least 95%, at least 90%, at least 85%, at least 80%, or at least 70% nucleotide sequence identity to a nucleotide sequence selected from the group consisting of the nucleotide sequences complementary to one of SEQ ID NOs.: 8-3795 and fragments comprising at least 10, 15, 20, 25, 30, 35, 40, 50, 75, 100, 150, 200, 300, 400, or 500 consecutive nucleotides thereof. Identity may be measured using BLASTN version 2.0 with the default parameters or tBLASTX with the default parameters. (Altschul, S. F. et al. Gapped BLAST and PSI-BLAST: A New Generation of Protein Database Search Programs, Nucleic Acid Res. 25: 3389-3402 (1997), the disclosure of which is incorporated herein by reference in its entirety) Alternatively a “homologuous coding nucleic acid” could be identified by membership of the gene of interest to a functional orthologue cluster. All other members of that orthologue cluster would be considered homologues. Such a library of functional orthologue clusters can be found at http://www.ncbi.nlm.nih.gov/COG. A gene can be classified into a cluster of orthologous groups or COG by using the COGNITOR program available at the above web site, or by direct BLASTP comparison of the gene of interest to the members of the COGs and analysis of these results as described by Tatusov, R. L., Galperin, M. Y., Natale, D. A. and Koonin, E. V. (2000) The COG database: a tool for genome-scale analysis of protein functions and evolution. Nucleic Acids Research v. 28 n. 1, pp33-36.

The term “homologous coding nucleic acid” also includes nucleic acids comprising nucleotide sequences which encode polypeptides having at least 99%, 95%, at least 90%, at least 85%, at least 80%, at least 70%, at least 60%, at least 50%, at least 40% or at least 25% maino acid identity or similarity to a polypeptide comprising the amino acid sequence of one of SEQ IDNOs: 3801-3805, 4861-5915, 10013-14110 or to a polypeptpide whose expression is inhibited by a nucleic acid comprising a nucleotide sequence of one of SEQ ID NOs: 8-3795 or fragments comprising at least 5, 10, 15, 20, 25, 30, 35, 40, 50, 75, 100, or 150 consecutive amino acids thereof as determined using the FASTA version 3.0t78 algorithm with the default parameters. Alternatively, protein identity or similarity may be identified using BLASTP with the default parameters, BLASTX with the default parameters, TBLASTN with the default parameters, or tBLASTX with the default parameters. (Altschul, S. F. et al. Gapped BLAST and PSI-BLAST: A New Generation of Protein Database Search Programs, Nucleic Acid Res.

25: 3389-3402 (1997), the disclosure of which is incorporated herein by reference in its entirety).

The term “homologous coding nucleic acid” also includes coding nucleic acids which hybridize under stringent conditions to a nucleic acid selected from the group consisting of the nucleotide sequences complementary to one of SEQ ID NOS.: 3796-3800, 3806-4860, 5916-10012 and coding nucleic acids comprising nucleotide sequences which hybridize under stringent conditions to a fragment comprising at least 10, 15, 20, 25, 30, 35, 40, 50, 75, 100, 150, 200, 300, 400, or 500 consecutive nucleotides of the sequences complementary to one of SEQ ID NOS.: 3796-3800, 3806-4860, 5916-10012 As used herein, “stringent conditions” means hybridization to filter-bound nucleic acid in 6× SSC at about 45° C. followed by one or more washes in 0.1× SSC/0.2% SDS at about 68° C. Other exemplary stringent conditions may refer, e.g., to washing in 6× SSC/0.05% sodium pyrophosphate at 37° C., 48° C., 55° C., and 60° C. as appropriate for the particular probe being used.

The term “homologous coding nucleic acid” also includes coding nucleic acids comprising nucleotide sequences which hybridize under moderate conditions to a nucleotide sequence selected from the group consisting of the sequences complementary to one of SEQ ID NOS.: 3796-3800, 3806-4860, 5916-10012 and coding nucleic acids comprising nucleotide sequences which hybridize under moderate conditions to a fragment comprising at least 10, 15, 20, 25, 30, 35, 40, 50, 75, 100, 150, 200, 300, 400, or 500 consecutive nucleotides of the sequences complementary to one of SEQ ID NOS.: 3796-3800, 3806-4860, 5916-10012. As used herein, “moderate conditions” means hybridization to filter-bound DNA in 6× sodium chloride/sodium citrate (SSC) at about 45° C. followed by one or more washes in 0.2× SSC/0.1% SDS at about 42-65° C.

The term “homologous coding nucleic acids” also includes nucleic acids comprising nucleotide sequences which encode a gene product whose activity may be complemented by a gene encoding a gene product whose activity is inhibited by a nucleic acid comprising a nucleotide sequence selected from the group consisting of SEQ ID NOs.: 8-3795. In some embodiments, the homologous coding nucleic acids may encode a gene product whose activity is complemented by the gene product encoded by a nucleic acid comprising a nucleotide sequence selected from the group consisting of SEQ ID NOS.: 3796-3800, 3806-4860, 5916-10012. In other embodiments, the homologous coding nucleic acids may comprise a nucleotide sequence encode a gene product whose activity is complemented by one of the polypeptides of SEQ ID NOs. 3745-4773.

The term “homologous antisense nucleic acid” includes nucleic acids comprising a nucleotide sequence having at least 97%, at least 95%, at least 90%, at least 85%, at least 80%, or at least 70% nucleotide sequence identity to a nucleotide sequence selected from the group consisting of one of the sequences of SEQ ID NOS. 8-3795 and fragments comprising at least 10, 15, 20, 25, 30, 35, 40, 50, 75, 100, 150, 200, 300, 400, or 500 consecutive nucleotides thereof. Homologous antisense nucleic acids may also comprising nucleotide sequences which have at least 97%, at least 95%, at least 90%, at least 85%, at least 80%, or at least 70% nucleotide sequence identity to a nucleotide sequence selected from the group consisting of the sequences complementary to one of sequences of SEQ ID NOS.: 3796-3800, 3806-4860, 5916-10012 and fragments comprising at least 10, 15, 20, 25, 30, 35, 40, 50, 75, 100, 150, 200, 300, 400, or 500 consecutive nucleotides thereof. Nucleic acid identity may be determined as described above.

The term “homologous antisense nucleic acid” also includes antisense nucleic acids comprising nucleotide sequences which hybridize under stringent conditions to a nucleotide sequence complementary to one of SEQ ID NOs.: 8-3795 and antisens nucleic acids comprising nucleotide sequences which hybridize under stringent conditions to a fragment comprising at least 10, 15, 20, 25, 30, 35, 40, 50, 75, 100, 150, 200, 300, 400, or 500 consecutive nucleotides of the sequence complementary to one of SEQ ID NOs. 8-3795. Homologous antisense nucleic acids also include antisense nucleic acids comprising nucleotide sequences which hybridize under stringent conditions to a nucleotide sequence selected from the group consisting of SEQ ID NOS.: 3796-3800, 3806-4860, 5916-10012 and antisense nucleic acids comprising nucleotide sequences which hybridize under stringent conditions to a fragment comprising at least 10, 15, 20,25, 30, 35, 40, 50, 75, 100, 150, 200, 300, 400, or 500 consecutive nucleotides of one of SEQ ID NOS.: 3796-3800, 3806-4860, 5916-10012.

The term “homologous antisense nucleic acid” also includes antisense nucleic acids comprising nucleotide sequences which hybridize under moderate conditions to a nucleotide sequence complementary to one of SEQ ID NOs.: 8-3795 and antisens nucleic acids comprising nucleotide seuqences which hybridize under moderate conditions to a fragment comprising at least 10, 15,20, 25, 30, 35, 40, 50, 75, 100, 150, 200, 300, 400, or 500 consecutive nucleotides of the sequence complementary to one of SEQ ID NOs. 8-3795. Homologous antisense nucleic acids also include antisense nucleic acids comprising nucleotide seuqences which hybridize under moderate conditions to a nucleotide sequence selected from the group consisting of SEQ ID NOS.: 3796-3800, 3806-4860, 5916-10012 and antisense nucleic acids which comprising nucleotide sequences hybridize under moderate conditions to a fragment comprising at least 10, 15, 20, 25, 30, 35, 40, 50, 75, 100, 150, 200, 300, 400, or 500 consecutive nucleotides of one of SEQ ID NOS.: 3796-3800, 3806-4860, 5916-10012.

By “homologous polypeptide” is meant a polypeptide homologous to a polypeptide whose activity or level is inhibited by a nucleic acid comprising a nucleotide sequence selected from the group consisting of SEQ ID NOs.: 8-3795 or by a homologous antisense nucleic acid. The term “homologous polypeptide” includes polypeptides having at least 99%, 95%, at least 90%, at least 85%, at least 80%, at least 70%, at least 60%, at least 50%, at least 40% or at least 25% amino acid identity or similarity to a polypeptide whose activity or level is inhibited by a nucleic acid selected from the group consisting of SEQ ID NOs: 8-3795 or by a homologous antisense nucleic acid, or polypeptides having at least 99%, 95%, at least 90%, at least 85%, at least 80%, at least 70%, at least 60%, at least 50%, at least 40% or at least 25% amino acid identity or similarity to a polypeptide to a fragment comprising at least 5, 10, 15, 20, 25, 30, 35, 40, 50, 75, 100, or 150 consecutive amino acids of a polypeptide whose activity or level is inhibited by a nucleic acid selected from the group consisting of SEQ ID NOs.: 8-3795 or by a homologous antisense nucleic acid. Identity or similarity may be determined using the FASTA version 3.0t78 algorithm with the default parameters. Alternatively, protein identity or similarity may be identified using BLASTP with the default parameters, BLASTX with the default parameters, or TBLASTN with the default parameters. (Altschul, S. F. et al. Gapped BLAST and PSI-BLAST: A New Generation of Protein Database Search Programs, Nucleic Acid Res. 25: 3389-3402 (1997), the disclosure of which is incorporated herein by reference in its entirety).

The term homologous polypeptide also includes polypeptides having at least 99%, 95%, at least 90%, at least 85%, at least 80%, at least 70%, at least 60%, at least 50%, at least 40% or at least 25% amino acid identity or similarity to a polypeptide selected from the group consisting of SEQ ID NOs: 3801-3805, 4861-5915, 10013-14110 and polypeptides having at least 99%, 95%, at least 90%, at least 85%, at least 80%, at least 70%, at least 60%, at least 50%, at least 40% or at least 25% amino acid identity or similarity to a fragment comprising at least 5, 10, 15, 20, 25, 30, 35, 40, 50, 75, 100, or 150 consecutive amino acids of a polypeptide selected from the group consisting of SEQ ID NOs: 3801-3805, 4861-5915, 10013-14110.

The invention also includes polynucleotides, preferably DNA molecules, that hybridize to one of the nucleic acids of SEQ ID NOs.: 8-3795, SEQ ID NOs.: 3796-3800, 3806-4860, 5916-10012 or the complements of any of the preceding nucleic acids. Such hybridization may be under stringent or moderate conditions as defined above or under other conditions which permit specific hybridization. The nucleic acid molecules of the invention that hybridize to these DNA sequences include oligodeoxynucleotides (“oligos”) which hybridize to the target gene under highly stringent or stringent conditions. In general, for oligos between 14 and 70 nucleotides in length the melting temperature (Tm) is calculated using the formula:

Tm(° C.)=81.5+16.6(log[monovalent cations (molar)]+0.41 (% G+C)−(500/N)

where N is the length of the probe. If the hybridization is carried out in a solution containing formamide, the melting temperature may be calculated using the equation:

Tm(° C.)=81.5+16.6(log[monovalent cations (molar)]+0.41(% G+C)−(0.61) (% formamide)−(500/N)

where N is the length of the probe. In general, hybridization is carried out at about 20-25 degrees below Tm (for DNA-DNA hybrids) or about 10-15 degrees below Tm (for RNA-DNA hybrids).

Other hybridization conditions are apparent to those of skill in the art (see, for example, Ausubel, F. M. et al., eds., 1989, Current Protocols in Molecular Biology, Vol. I, Green Publishing Associates, Inc. and John Wiley & Sons, Inc., New York, at pp. 6.3.1-6.3.6 and 2.10.3, the disclosure of which is incorporated herein by reference in its entirety).

The term, Salmonella, is the generic name for a large group of gram-negative enteric bacteria that are closely related to Escherichia coli. The diseases caused by Salmonella are often due to contamination of foodstuffs or the water supply and affect millions of people each year. Traditional methods of Salmonella taxonomy were based on assigning a separate species name to each serologically distinguishable strain (Kauffmann, F 1966 The bacteriology of the Enterobacteriaceae. Munksgaard, Copenhagen). Serology of Salmonella is based on surface antigens (O [somatic] and H [flagellar]). Over 2,400 serotypes or serovars of Salmonella are known (Popoff, et al. 2000 Res. Microbiol. 151:63-65). Therefore, each serotype was considered to be a separate species and often given names, accordingly (e.g. S. paratyphi, S. typhimurium, S. typhi, S. enteriditis, etc.).

However, by the 1970s and 1980s it was recognized that this system was not only cumbersome, but also inaccurate. Then, many Salmonella species were lumped into a single species (all serotypes and subgenera I, II, and IV and all serotypes of Arizona) with a second subspecies, S. bongorii also recognized (Crosa, et al., 1973, J. Bacteriol. 115:307-315). Though species designations are based on the highly variable surface antigens, the Salmonella are very similar otherwise with a major exception being pathogenicity determinants.

There has been some debate on the correct name for the Salmonella species. Currently (Brenner, et al. 2000 J. Clin. Microbiol. 38:2465-2467), the accepted name is Salmonella enterica. S. enterica is divided into six subspecies (I, S. enterica subsp. enterica; II, S. enterica, subsp. salamae; IIIa, S. enterica subsp. arizonae; IIIb, S. enterica subsp. diarizonae; IV, S. enterica subsp. houtenae; and VI, S. enterica subsp. indica). Within subspecies I, serotypes are used to distinguish each of the serotypes or serovars (e.g. S. enterica serotype Enteriditis, S. enterica serotype Typhimurium, S. enterica serotype Typhi, and S. enterica serotype Choleraesuis, etc.). Current convention is to spell this out on first usage (Salmonella enterica ser. Typhimurium) and then use an abbreviated form (Salmonella Typhimurium or S. Typhimurium). Note, the genus and species names (Salmonella enterica) are italicized but not the serotype/serovar name (Typhimurium). Because the taxonomic committees have yet to officially approve of the actual species name, this latter system is what is employed by the CDC (Brenner, et al. 2000 J. Clin. Microbiol. 38:2465-2467). Due to the concerns of both taxonomic priority and medical importance, some of these serotypes might ultimately receive full species designations (S. typhi would be the most notable).

Therefore, as used herein “Salmonella enterica or S. enterica” includes serovars Typhi, Typhimurium, Paratyphi, Choleraesuis, etc.” However, appeals of the “official” name are in process and the taxonomic designations may change (S. choleraesuis is the species name that could replace S. enterica based solely on priority).

By “identifying a compound” is meant to screen one or more compounds in a collection of compounds such as a combinatorial chemical library or other library of chemical compounds or to characterize a single compound by testing the compound in a given assay and determining whether it exhibits the desired activity.

By “inducer” is meant an agent or solution which, when placed in contact with a cell or microorganism, increases transcription, or inhibitor and/or promoter clearance/fidelity, from a desired promoter.

As used herein, “nucleic acid” means DNA, RNA, or modified nucleic acids. Thus, the terminology “the nucleic acid of SEQ ID NO: X” or “the nucleic acid comprising the nucleotide sequence” includes both the DNA sequence of SEQ ID NO: X and an RNA sequence in which the thymidines in the DNA sequence have been substituted with uridines in the RNA sequence and in which the deoxyribose backbone of the DNA sequence has been substituted with a ribose backbone in the RNA sequence. Modified nucleic acids are nucleic acids having nucleotides or structures which do not occur in nature, such as nucleic acids in which the internucleotide phosphate residues with methylphosphonates, phosphorothioates, phosphoramidates, and phosphate esters. Nonphosphate internucleotide analogs such as siloxane bridges, carbonate brides, thioester bridges, as well as many others known in the art may also be used in modified nucleic acids. Modified nucleic acids may also comprise, (x-anomeric nucleotide units and modified nucleotides such as 1,2-dideoxy-d-ribofuranose, 1,2-dideoxy-1-phenylribofuranose, and N⁴, N⁴-ethano-5-methyl-cytosine are contemplated for use in the present invention. Modified nucleic acids may also be peptide nucleic acids in which the entire deoxyribose-phosphate backbone has been exchanged with a chemically completely different, but structurally homologous, polyamide (peptide) backbone containing 2-aminoethyl glycine units.

As used herein, “sub-lethal” means a concentration of an agent below the concentration required to inhibit all cell growth.

### BRIEF DESCRIPTION OF THE DRAWINGS

FIG. 1 is an IPTG dose response curve in E. coli transformed with an IPTG-inducible plasmid containing either an antisense clone to the E. coli ribosomal protein rplW (AS-rplW) which is required for protein synthesis and essential for cell proliferation, or an antisense clone to the elaD (AS-elaD) gene which is not known to be involved in protein synthesis and which is also essential for proliferation.

FIG. 2A is a tetracycline dose response curve in E. coli transformed with an IPTG-inducible plasmid containing antisense to rplW (AS-rplW) in the absence (0) or presence of IPTG at concentrations that result in 20% and 50% growth inhibition.

FIG. 2B is a tetracycline dose response curve in E. coli transformed with an IPTG-inducible plasmid containing antisense to elaD (AS-elaD)in the absence (0) or presence of IPTG at concentrations that result in 20% and 50% growth inhibition.

FIG. 3 is a graph showing the fold increase in tetracycline sensitivity of E. coli transfected with antisense clones to essential ribosomal proteins L23 (AS-rplW) and L7/L12 and L10 (AS-rplLrplJ). Antisense clones to genes known to not be directly involved in protein synthesis, atpB/E (AS-atpB/E), visC (AS-visC), elaD (AS-elaD), yohH (AS-yohH), are much less sensitive to tetracycline.

FIG. 4 illustrates the results of an assay in which Staphylococcus aureus cells transcribing an antisense nucleic acid complementary to the gyrB gene encoding the β subunit of gyrase were contacted with several antibiotics whose targets were known.

### DETAILED DESCRIPTION OF THE PREFERRED EMBODIMENTS

The present invention describes a group of prokaryotic genes and gene families required for cellular proliferation. Exemplary genes and gene families from Staphylococcus aureus, Salmonella typhimurium, Klebsiella pneumoniae, Pseudomonas aeruginosa and Enterococcus faecalis, Escherichia coli, Enterococcus faecalis, Haemophilus influenzae, Helicobacter pylori, Klebsiella pneumoniae, Pseudomonas aeruginosa, Staphylococcus aureus, and Salmonella typhi are provided. A proliferation-required gene or gene family is one where, in the absence or substantial reduction of a gene transcript and/or gene product, growth or viability of the cell or microorganism is reduced or eliminated. Thus, as used herein, the terminology “proliferation-required” or “required for proliferation” encompasses instances where the absence or substantial reduction of a gene transcript and/or gene product completely eliminates cell growth as well as instances where the absence of a gene transcript and/or gene product merely reduces cell growth. These proliferation-required genes can be used as potential targets for the generation of new antimicrobial agents. To achieve that goal, the present invention also encompasses assays for analyzing proliferation-required genes and for identifying compounds which interact with the gene and/or gene products of the proliferation-required genes. In addition, the present invention contemplates the expression of genes and the purification of the proteins encoded by the nucleic acid sequences identified as required proliferation genes and reported herein. The purified proteins can be used to generate reagents and screen small molecule libraries or other candidate compound libraries for compounds that can be further developed to yield novel antimicrobial compounds.

The present invention also describes methods for identification of nucleotide sequences homologous to these genes and polypeptides described herein, including nucleic acids comprising nucleotide sequences homologous to the nucleic acids of SEQ ID NOS.: 3796-3800, 3806-4860, 5916-10012 and polypeptides homologous to the polypeptides of SEQ ID NOs.: 3801-3805, 4861-5915, 10013-14110. For example, these sequences may be used to identify homologous coding nucleic acids, homologous antisense nucleic acids, or homologous polypeptides in microorganisms such as Anaplasma marginale, Aspergillus fumigatus, Bacillus anthracis, Bacterioides fragilis Bordetella pertussis, Burkholderia cepacia, Campylobacter jejuni, Candida albicans, Candida glabrata (also called Torulopsis glabrata), Candida tropicalis, Candida parapsilosis, Candida guilliermondii, Candida krusei, Candida kefyr (also called Candida pseudotropicalis), Candida dubliniensis, Chlamydia pneumoniae, Chlamydia trachomatus, Clostridium botulinum, Clostridium difficile, Clostridium perfringens, Coccidiodes immitis, Corynebacterium diptheriae, Cryptococcus neoformans, Enterobacter cloacae, Enterococcus faecalis, Enterococcus faecium, Escherichia coli, Haemophilus influenzae, Helicobacter pylori, Histoplasma capsulatum, Klebsiella pneumoniae, Listeria monocytogenes, Mycobacterium leprae, Mycobacterium tuberculosis, Neisseria gonorrhoeae, Neisseria meningitidis, Nocardia asteroides, Pasteurella haemolytica, Pasteurella multocida, Pneumocystis carinii, Proteus vulgaris, Pseudomonas aeruginosa, Salmonella bongori, Salmonella cholerasuis, Salmonella enterica, Salmonella paratyphi, Salmonella typhi, Salmonella typhimurium, Staphylococcus aureus, Listeria monocytogenes, Moxarella catarrhalis, Shigella boydii, Shigella dysenteriae, Shigella flexneri, Shigella sonnei, Staphylococcus epidermidis, Streptococcus pneumoniae, Streptococcus mutans, Treponema pallidum, Yersinia enterocolitica, Yersinia pestis or any species falling within the genera of any of the above species. In some embodiments, the homologous coding nucleic acids, homologus antisense nucleic acids, or homologous polypeptides are identified in an organism other than E. coli.

The homologous coding nucleic acids, homologous antisense nucleic acids, or homologous polypeptides, may then be used in each of the methods described herein, including methods to identify compounds which inhibit the proliferation of the organism containing the homologous coding nucleic acid, homologous antisense nucleic acid or homologous polypeptide, methods of inhibiting the growth of the organism containing the homologous coding nucleic acid, homologus antisense nucleic acid or homologous polypeptide, methods of identifying compounds which influence the activity or level of a gene product required for proliferation of the organism containing the homologous coding nucleic acid, homologous antisense nucleic acid or homologous polypeptide, methods for identifying compounds or nucleic acids having the ability to reduce the level or activity of a gene product required for proliferation of the organism containing the homologous coding nucleic acid, homologous antisense nucleic acid or homologous polypeptide, methods of inhibiting the activity or expression of a gene in an operon required for proliferation of the organism containing the homologous coding nucleic acid, homologous antisense nucleic acid or homologous polypeptide, methods for identifying a gene required proliferation of the organism containing the homologous coding nucleic acid, homologous antisense nucleic acid or homologous polypeptide, methods for identifying the biological pathway in which a gene or gene product required for proliferation of the organism containing the homologous coding nucleic acid, homologous antisense nucleic acid or homologous polypeptide lies, methods for identifying compounds having activity against biological pathway required for proliferation of the organism containing the homologous coding nucleic acid, homologous antisense nucleic acid or homologous polypeptide, methods for determining the biological pathway on which a test compound acts, and methods of inhibiting the proliferation of the organism containing the homologous coding nucleic acid, homologous antisense nucleic acid or homologous polypeptide in a subject. In some embodiments of the present invention, the methods are performed using an organism, other than E. coli or a gene or gene product from an organism other than E. coli.

The present invention utilizes a novel method to identify proliferation-required sequences. Generally, a library of nucleic acid sequences from a given source are subcloned or otherwise inserted immediately downstream of an inducible promoter on an appropriate vector, such as a Staphylococcus aureus/E. coli or Pseudomonas aeruginosa/E. coli shuttle vector, or a vector which will replicate in both Salmonella typhimurium and Klebsiella pneumoniae, or other vector or shuttle vector capable of functioning in the intended organism., thus forming an expression library. It is generally preferred that expression is directed by a regulatable promoter sequence such that expression level can be adjusted by addition of variable concentrations of an inducer molecule or of an inhibitor molecule to the medium. Temperature activated promoters, such as promoters regulated by temperature sensitive repressors, such as the lambda C₁₈₅₇ repressor, are also envisioned. Although the insert nucleic acids may be derived from the chromosome of the cell or microorganism into which the expression vector is to be introduced, because the insert is not in its natural chromosomal location, the insert nucleic acid is an exogenous nucleic acid for the purposes of the discussion herein. The term “expression” is defined as the production of a sense or antisense RNA molecule from a gene, gene fragment, genomic fragment, chromosome, operon or portion thereof. Expression can also be used to refer to the process of peptide or polypeptide synthesis. An expression vector is defined as a vehicle by which a ribonucleic acid (RNA) sequence is transcribed from a nucleic acid sequence carried within the expression vehicle. The expression vector can also contain features that permit translation of a protein product from the transcribed RNA message expressed from the exogenous nucleic acid sequence carried by the expression vector. Accordingly, an expression vector can produce an RNA molecule as its sole product or the expression vector can produce a RNA molecule that is ultimately translated into a protein product.

Once generated, the expression library containing the exogenous nucleic acid sequences is introduced into a population of cells (such as the organism from which the exogenous nucleic acid sequences were obtained) to search for genes that are required for bacterial proliferation. Because the library molecules are foreign, in context, to the population of cells, the expression vectors and the nucleic acid segments contained therein are considered exogenous nucleic acid.

Expression of the exogenous nucleic acid fragments in the test population of cells containing the expression library is then activated. Activation of the expression vectors consists of subjecting the cells containing the vectors to conditions that result in the expression of the exogenous nucleic acid sequences carried by the expression library. The test population of cells is then assayed to determine the effect of expressing the exogenous nucleic acid fragments on the test population of cells. Those expression vectors that negatively impacted the growth of the cells upon induction of expression of the random sequences contained therein were identified, isolated, and purified for further study.

A variety of assays are contemplated to identify nucleic acid sequences that negatively impact growth upon expression. In one embodiment, growth in cultures expressing exogenous nucleic acid sequences and growth in cultures not expressing these sequences is compared. Growth measurements are assayed by examining the extent of growth by measuring optical densities. Alternatively, enzymatic assays can be used to measure bacterial growth rates to identify exogenous nucleic acid sequences of interest. Colony size, colony morphology, and cell morphology are additional factors used to evaluate growth of the host cells. Those cultures that fail to grow or grow at a reduced rate under expression conditions are identified as containing an expression vector encoding a nucleic acid fragment that negatively affects a proliferation-required gene.

Once exogenous nucleic acids of interest are identified, they are analyzed. The first step of the analysis is to acquire the nucleotide sequence of the nucleic acid fragment of interest. To achieve this end, the insert in those expression vectors identified as containing a nucleotide sequence of interest is sequenced, using standard techniques well known in the art. The next step of the process is to determine the source of the nucleotide sequence. As used herein “source” means the genomic region containing the cloned fragment.

Determination of the gene(s) corresponding to the nucleotide sequence was achieved by comparing the obtained sequence data with databases containing known protein and nucleotide sequences from various microorganisms. Thus, initial gene identification was made on the basis of significant sequence similarity or identity to either characterized or predicted Staphylococcus aureus, Salmonella typhimurium, Klebsiella pneumoniae, Pseudomonas aeruginosa or Enterococcus faecalis genes or their encoded proteins and/or homologues in other species.

The number of nucleotide and protein sequences available in database systems has been growing exponentially for years. For example, the complete nucleotide sequences of Caenorhabditis elegans and several bacterial genomes, including E. coli, Aeropyrum pernix, Aquifex aeolicus, Archaeoglobus fulgidus, Bacillus subtilis, Borrelia burgdorferi, Chlamydia pneumoniae, Chlamydia trachomatis, Clostridium tetani, Corynebacterium diptheria, Deinococcus radiodurans, Haemophilus influenzae, Helicobacter pylori 26695, Helicobacter pylori J99, Methanobacterium thermoautotrophicum, Methanococcus jannaschii, Mycobacterium tuberculosis, Mycoplasma genitalium, Mycoplasma pneumoniae, Pseudomonas aeruginosa, Pyrococcus abyssi, Pyrococcus horikoshii, Rickettsia prowazekii, Synechocystis PCC6803, Thermotoga maritima, Treponema pallidum, Bordetella pertussis, Campylobacter jejuni, Clostridium acetobutylicum, Mycobacterium tuberculosis CSU#93, Neisseria gonorrhoeae, Neisseria meningitidis, Pseudomonas aeruginosa, Pyrobaculum aerophilum, Pyrococcus furiosus, Rhodobacter capsulatus, Salmonella typhimurium, Streptococcus mutans, Streptococcus pyogenes, Ureaplasma urealyticum and Vibrio cholera are available. This nucleotide sequence information is stored in a number of databanks, such as GenBank, the National Center for Biotechnology Information (NCBI), the Genome Sequencing Center (http:Hlgenome.wustl.edu/gsc/salmonella.shtml), and the Sanger Centre (http://www.sanger.ac.uk/projects/S_typhi) which are publicly available for searching.

A variety of computer programs are available to assist in the analysis of the sequences stored within these databases. FASTA, (W. R. Pearson (1990) “Rapid and Sensitive Sequence Comparison with FASTP and FASTA” Methods in Enzymology 183:63-98), Sequence Retrieval System (SRS), (Etzold & Argos, SRS an indexing and retrieval tool for flat file data libraries. Comput. Appl. Biosci. 9:49-57, 1993) are two examples of computer programs that can be used to analyze sequences of interest. In one embodiment of the present invention, the BLAST family of computer programs, which includes BLASTN version 2.0 with the default parameters, or BLASTX version 2.0 with the default parameters, is used to analyze nucleotide sequences.

BLAST, an acronym for “Basic Local Alignment Search Tool,” is a family of programs for database similarity searching. The BLAST family of programs includes: BLASTN, a nucleotide sequence database searching program, BLASTX, a protein database searching program where the input is a nucleic acid sequence; and BLASTP, a protein database searching program. BLAST programs embody a fast algorithm for sequence matching, rigorous statistical methods for judging the significance of matches, and various options for tailoring the program for special situations. Assistance in using the program can be obtained by e-mail at blastincbi.nlm.nih.gov. tBLASTX can be used to translate a nucleotide sequence in all three potential reading frames into an amino acid sequence.

Bacterial genes are often transcribed in polycistronic groups. These groups comprise operons, which are a collection of genes and intergenic sequences under common regulation. The genes of an operon are transcribed on the same MRNA and are often related functionally. Given the nature of the screening protocol, it is possible that the identified exogenous nucleic acid corresponds to a gene or portion thereof with or without adjacent noncoding sequences, an intragenic sequence (i.e. a sequence within a gene), an intergenic sequence (i.e. a sequence between genes), a nucleotide sequence spanning at least a portion of two or more genes, a 5′ noncoding region or a 3′ noncoding region located upstream or downstream from the actual nucleotide sequence that is required for bacterial proliferation. Accordingly, it is often desirable to determine which gene(s) that is encoded within the operon is individually required for proliferation.

In one embodiment of the present invention, an operon is identified and then dissected to determine which gene or genes are required for proliferation. Operons can be identified by a variety of means known to those in the art. For example, the RegulonDB DataBase described by Huerta et al. (Nucl. Acids Res. 26:55-59, 1998), which may also be found on the website http://www.cifn.unam.mx/Computational_Biology/regulondb/, the disclosures of which are incorporated herein by reference in their entireties, provides information about operons in Escherichia coli. The Subtilist database (http://bioweb.pasteur.fr/GenoList/SubtiList), (Moszer, I., Glaser, P. and Danchin, A. (1995) Microbiology 141: 261-268 and Moszer, 1 (1998) FEBS Letters 430: 28-36, the disclosures of which are incorporated herein in their entireties), may also be used to predict operons. This database lists genes from the fully sequenced, Gram-positive bacteria, Bacillus subtilis, together with predicted promoters and terminator sites. This information can be used in conjunction with the Staphylococcus aureus genomic sequence data to predict operons and thus produce a list of the genes affected by the antisense nucleic acids of the present invention. The Pseudomonas aerginosa web site (http://www.pseudomonas.com) can be used to help predict operon organization in this bacterium. The databases available from the Genome Sequencing Center (http:/Hgenome.wustl.edu/gsc/salmonella.shtml), and the Sanger Centre (http:/Hwww.sanger.ac.uk/projects/S typhi) may be used to predict operons in Salmonella typhimurium. The TIGR microbial database has an incomplete version of the E. faecalis genome http://www.tigr.org/cgi-bin/BlastSearch/blast.cai?organism=_e faecalis. One can take a nucleotide sequence and BLAST it for homologs.

A number of techniques that are well known in the art can be used to dissect the operon. Analysis of RNA transcripts by Northern blot or primer extension techniques are commonly used to analyze operon transcripts. In one aspect of this embodiment, gene disruption by homologous recombination is used to individually inactivate the genes of an operon that is thought to contain a gene required for proliferation.

Several gene disruption techniques have been described for the replacement of a functional gene with a mutated, non-functional (null) allele. These techniques generally involve the use of homologous recombination. One technique using homologous recombination in Staphylococcus aureus is described in Xia et a. 1999, Plasmid 42: 144-149, the disclosure of which is incorporated herein by reference in its entirety. This technique uses crossover PCR to create a null allele with an in-frame deletion of the coding region of a target gene. The null allele is constructed in such a way that nucleotide sequences adjacent to the wild type gene are retained. These homologous sequences surrounding the deletion null allele provide targets for homologous recombination so that the wild type gene on the Staphylococcus aureus chromosome can be replaced by the constructed null allele. This method can be used with other bacteria as well, including Salmonella and Klebsiella species. Similar gene disruption methods that employ the counter selectable marker sacb (Schweizer, H. P., Klassen, T. and Hoang, T. (1996) Mol. Biol. of Pseudomonas. ASM press, 229-237, the disclosure of which is incorporated herein by reference in its entirety) are available for Pseudomonas, Salmonella and Klebsiella species. E. faecalis genes can be disrupted by recombining in a non-replicating plasmid that contains an internal fragment to that gene (Leboeuf, C., L. Leblanc, Y. Auffray and A. Hartke. 2000. J. Bacteriol. 182:5799-5806, the disclosure of which is incorporated herein by reference in its entirety).

The crossover PCR amplification product is subcloned into a suitable vector having a selectable marker, such as a drug resistance marker. In some embodiments the vector may have an origin of replication which is functional in E. coli or another organism distinct from the organism in which homologous recombination is to occur, allowing the plasmid to be grown in E. coli or the organism other than that in which homologous recombination is to occur, but may lack an origin of replication functional in Staphylococcus aureus, Salmonella typhimurium, Klebsiella pneumoniae, Pseudomonas aeruginosa and Enterococcus faecalis, Escherichia coli, Enterococcus faecalis, Haemophilus influenzae, Helicobacter pylori, Klebsiella pneumoniae, Pseudomonas aeruginosa, Staphylococcus aureus, or Salmonella typhi such that selection of the selectable marker requires integration of the vector into the homologous region of the Staphylococcus aureus, Salmonella typhimurium, Klebsiella pneumoniae, Pseudomonas aeruginosa and Enterococcus faecalis, Escherichia coli, Enterococcus faecalis, Haemophilus influenzae, Helicobacter pylori, Klebsiella pneumoniae, Pseudomonas aeruginosa, Staphylococcus aureus, or Salmonella typhi chromosome. Usually a single crossover event is responsible for this integration event such that the Staphylococcus aureus, Salmonella typhimurium, Klebsiella pneumoniae, Pseudomonas aeruginosa and Enterococcus faecalis, Escherichia coli, Enterococcus faecalis, Haemophilus influenzae, Helicobacter pylori, Klebsiella pneumoniae, Pseudomonas aeruginosa, Staphylococcus aureus, or Salmonella typhi chromosome now contains a tandem duplication of the target gene consisting of one wild type allele and one deletion null allele separated by vector sequence. Subsequent resolution of the duplication results in both removal of the vector sequence and either restoration of the wild type gene or replacement by the in-frame deletion. The latter outcome will not occur if the gene should prove essential. A more detailed description of this method is provided in Example 5 below. It will be appreciated that this method may be practiced with any of the nucleic acids or organisms described herein.

Recombinant DNA techniques can be used to express the entire coding sequences of the gene identified as required for proliferation, or portions thereof. The over-expressed proteins can be used as reagents for further study. The identified exogenous sequences are isolated, purified, and cloned into a suitable expression vector using methods well known in the art. If desired, the nucleic acids can contain the nucleotide sequences encoding a signal peptide to facilitate secretion of the expressed protein.

Expression of fragments of the bacterial genes identified as required for proliferation is also contemplated by the present invention. The fragments of the identified genes can encode a polypeptide comprising at least 5, at least 10, at least 15, at least 20, at least 25, at least 30, at least 35, at least 40, at least 45, at least 50, at least 55, at least 60, at least 65, at least 75, or more than 75 consecutive amino acids of a gene complementary to one of the identified sequences of the present invention. The nucleic acids inserted into the expression vectors can also contain endogenous sequences upstream and downstream of the coding sequence.

When expressing the encoded protien of the idnetified required for bacterial proliferation or a fragment thereof, the nucleotide sequence to be expressed is operably linked to a promoter in an expression vector using conventional cloning technology. The expression vector can be any of the bacterial, insect, yeast, or mammalian expression systems known in the art. Commercially available vectors and expression systems are available from a variety of suppliers including Genetics Institute (Cambridge, Mass.), Stratagene (La Jolla, Calif.), Promega (Madison, Wis.), and Invitrogen (San Diego, Calif.). If desired, to enhance expression and facilitate proper protein folding, the codon usage and codon bias of the sequence can be optimized for the particular expression organism in which the expression vector is introduced, as explained by Hatfield, et al., U.S. Pat. No. 5,082,767, incorporated herein by this reference. Fusion protein expression systems are also contemplated by the present invention.

Following expression of the protein encoded by the identified exogenous nucleic acid, the protein may be purified. Protein purification techniques are well known in the art. Proteins encoded and expressed from identified exogenous nucleic acids can be partially purified using precipitation techniques, such as precipitation with polyethylene glycol. Alternatively, epitope tagging of the protein can be used to allow simple one step purification of the protein. In addition, chromatographic methods such as ion-exchange chromatography, gel filtration, use of hydroxyapaptite columns, immobilized reactive dyes, chromatofocusing, and use of high-performance liquid chromatography, may also be used to purify the protein. Electrophoretic methods such as one-dimensional gel electrophoresis, high-resolution two-dimensional polyacrylamide electrophoresis, isoelectric focusing, and others are contemplated as purification methods. Also, affinity chromatographic methods, comprising antibody columns, ligand presenting columns and other affinity chromatographic matrices are contemplated as purification methods in the present invention.

The purified proteins produced from the gene coding sequences identified as required for proliferation can be used in a variety of protocols to generate useful antimicrobial reagents. In one embodiment of the present invention, antibodies are generated against the proteins expressed from the identified exogenous nucleic acids. Both monoclonal and polyclonal antibodies can be generated against the expressed proteins. Methods for generating monoclonal and polyclonal antibodies are well known in the art. Also, antibody fragment preparations prepared from the produced antibodies discussed above are contemplated.

In addition, the purified protein, fragments thereof, or derivatives thereof may be administered to an individual in a pharmaceutically acceptable carrier to induce an immune response against the protein. Preferably, the immune response is a protective immune response which protects the individual. Methods for determining appropriate dosages of the protein and pharmaceutically acceptable carriers may be determined empiracally and are familiar to those skilled in the art.

Another application for the purified proteins of the present invention is to screen small molecule libraries for candidate compounds active against the various target proteins of the present invention. Advances in the field of combinatorial chemistry provide methods, well known in the art, to produce large numbers of candidate compounds that can have a binding, or otherwise inhibitory effect on a target protein. Accordingly, the screening of small molecule libraries for compounds with binding affinity or inhibitory activity for a target protein produced from an identified gene is contemplated by the present invention.

The present invention further contemplates utility against a variety of other pathogenic microorganisms in addition to Staphylococcus aureus, Salmonella typhimurium, Klebsiella pneumoniae, Pseudomonas aeruginosa and Enterococcus faecalis, Escherichia coli, Enterococcus faecalis, Haemophilus influenzae, Helicobacter pylori, Klebsiella pneumoniae, Pseudomonas aeruginosa, Staphylococcus aureus, or Salmonella typhi. For example, homologous coding nucleic acids, homologous antisense nucleic acids or homologous polypeptides from other pathogenic microorganisms (including nucleic acids homologous to the nucleic acids of SEQ ID NOs.: 3796-3800, 3806-4860, 5916-10012, nucleic acids homologous to the antisense nucleic acids of SEQ ID NOs.: 8-3795, and polypeptides homologous to the polypeptides of SEQ ID NOs.: 3801-3805, 4861-5915, 10013-14110) may be identified using methods such as those described herein. The homologous coding nucleic acids, homologous antisense nucleic acids or homologous polypeptides may be used to identify compounds which inhibit the proliferation of these other pathogenic microorganisms using methods such as those described herein.

For example, the proliferation-required nucleic acids, antisense nucleic acids, and polypeptides from Staphylococcus aureus, Salmonella typhimurium, Klebsiella pneumoniae, Pseudomonas aeruginosa and Enterococcus faecalis, Escherichia coli, Enterococcus faecalis, Haemophilus influenzae, Helicobacter pylori, Klebsiella pneumoniae, Pseudomonas aeruginosa, Staphylococcus aureus, or Salmonella typhi described herein (including the nucleic acids of SEQ ID NOs.: 3796-3800, 3806-4860, 5916-10012, the antisense nucleic acids of SEQ ID NOs: 8-3795, and the polypeptides of SEQ ID NOs.: 3801-3805, 4861-5915, 10013-14110) may be used to identify homologous coding nucleic acids, homologous antisense nucleic acids or homologous polypeptides required for proliferation in prokaryotes and eukaryotes. For example, nucleic acids or polypeptides required for the proliferation of protists, such as Plasmodium spp.; plants; animals, such as Entamoeba spp. and Contracaecum spp; and fungi including Candida spp., (e.g., Candida albicans), Cryptococcus neoformans, and Aspergillus fumigatus may be identified. In one embodiment of the present invention, monera, specifically bacteria, including both Gram positive and Gram negative bacteria, are probed in search of novel gene sequences required for proliferation. Likewise, homologous antisense nucleic acids which may be used to inhibit growth of these organisms or to identify antibiotics may also be identified. These embodiments are particularly important given the rise of drug resistant bacteria.

The number of bacterial species that are becoming resistant to existing antibiotics is growing. A partial list of these microorganisms includes: Escherichia spp., such as E. coli, Enterococcus spp, such as E. faecalis; Pseudomonas spp., such as P. aeruginosa, Clostridium spp., such as C. botulinum, Haemophilus spp., such as H. influenzae, Enterobacter spp., such as E. cloacae, Vibrio spp., such as V. cholera; Moraxala spp., such as M. catarrhalis; Streptococcus spp., such as S. pneumoniae, Neisseria spp., such as N. gonorrhoeae; Mycoplasma spp., such as Mycoplasma pneumoniae; Salmonella typhimurium; Helicobacter pylori; Escherichia coli; and Mycobacterium tuberculosis. The genes and polypeptides identified as required for the proliferation of Staphylococcus aureus, Salmonella typhimurium, Klebsiella pneumoniae, Pseudomonas aeruginosa and Enterococcus faecalis, Escherichia coli, Enterococcus faecalis, Haemophilus influenzae, Helicobacter pylori, Klebsiella pneumoniae, Pseudomonas aeruginosa, Staphylococcus aureus, or Salmonella typhi (including the nucleic acids of SEQ ID NOs.: 3796-3800, 3806-4860, 5916-10012, the sequences complementary to the nucleic acids of SEQ ID NOs.: 3796-3800, 3806-4860, 5916-10012, and the polypeptides of SEQ ID NOs.: 3801-3805, 4861-5915, 10013-14110) can be used to identify homologous coding nucleic acids or homologous polypeptides required for proliferation from these and other organisms using methods such as nucleic acid hybridization and computer database analysis. Likewise, the antisense nucleic acids which inhibit proliferation of Staphylococcus aureus, Salmonella typhimurium, Klebsiella pneumoniae, Pseudomonas aeruginosa and Enterococcus faecalis, Escherichia coli, Enterococcus faecalis, Haemophilus influenzae, Helicobacter pylori, Klebsiella pneumoniae, Pseudomonas aeruginosa, Staphylococcus aureus, or Salmonella typhi (including the antisense nucleic acids of SEQ ID NOs.: 8-3795 or the sequences complementary thereto) may also be used to identify antisense nucleic acids which inhibit proliferation of these and other microorganisms or cells using nucleic acid hybridization or computer database analysis.

In one embodiment of the present invention, the nucleic acid sequences from Staphylococcus aureus, Salmonella typhimurium, Klebsiella pneumoniae, Pseudomonas aeruginosa and Enterococcus faecalis, Escherichia coli, Enterococcus faecalis, Haemophilus influenzae, Helicobacter pylori, Klebsiella pneumoniae, Pseudomonas aeruginosa, Staphylococcus aureus, or Salmonella typhii (including the nucleic acids of SEQ ID NOs.: 3796-3800, 3806-4860, 5916-10012 and the antisense nucleic acids of SEQ ID NOs. 8-3795) are used to screen genomic libraries generated from Staphylococcus aureus, Salmonella typhimurium, Klebsiella pneumoniae, Pseudomonas aeruginosa and Enterococcus faecalis, Escherichia coli, Enterococcus faecalis, Haemophilus influenzae, Helicobacter pylori, Klebsiella pneumoniae, Pseudomonas aeruginosa, Staphylococcus aureus, or Salmonella typhi and other bacterial species of interest. For example, the genomic library may be from Gram positive bacteria, Gram negative bacteria or other organisms including Anaplasma marginale, Aspergillus fumigatus, Bacillus anthracis, Bacterioides fragilis Bordetella pertussis, Burkholderia cepacia, Campylobacter jejuni, Candida albicans, Candida glabrata (also called Torulopsis glabrata), Candida tropicalis, Candida parapsilosis, Candida guilliermondii, Candida krusei, Candida kefyr (also called Candida pseudotropicalis), Candida dubliniensis, Chlamydia pneumoniae, Chlamydia trachomatus, Clostridium botulinum, Clostridium difficile, Clostridium perfringens, Coccidiodes immitis, Corynebacterium diptheriae, Cryptococcus neoformans, Enterobacter cloacae, Enterococcus faecalis, Enterococcus faecium, Escherichia coli, Haemophilus influenzae, Helicobacter pylori, Histoplasma capsulatum, Klebsiella pneumoniae, Listeria monocytogenes, Mycobacterium leprae, Mycobacterium tuberculosis, Neisseria gonorrhoeae, Neisseria meningitidis, Nocardia asteroides, Pasteurella haemolytica, Pasteurella multocida, Pneumocystis carinii, Proteus vulgaris, Pseudomonas aeruginosa, Salmonella bongori, Salmonella cholerasuis, Salmonella enterica, Salmonella paratyphi, Salmonella typhi, Salmonella typhimurium, Staphylococcus aureus, Listeria monocytogenes, Moxarella catarrhalis, Shigella boydii, Shigella dysenteriae, Shigella flexneri, Shigella sonnei, Staphylococcus epidermidis, Streptococcus pneumoniae, Streptococcus mutans, Treponema pallidum, Yersinia enterocolitica, Yersinia pestis or any species falling within the genera of any of the above species, including coagulase negative species of Staphylococcus. In some embodiments, the genomic library may be from an organism other than E. coli. Standard molecular biology techniques are used to generate genomic libraries from various cells or microorganisms. In one aspect, the libraries are generated and bound to nitrocellulose paper. The identified exogenous nucleic acid sequences of the present invention can then be used as probes to screen the libraries for homologous sequences.

For example, the libraries may be screened to identify homologous coding nucleic acids or homologous antisense nucleic acids comprising nucleotide sequences which hybridize under stringent conditions to a nucleic acid selected from the group consisting of SEQ ID NOs.: 8-3795, nucleic acids comprising nucleotide sequences which hybridize under stringent conditions to a fragment comprising at least 10, 15, 20, 25, 30, 35, 40, 50, 75, 100, 150, 200,300, 400, or 500 consecutive nucleotides of one of SEQ ID .NOs. 8-3795, nucleic acids comprising nucleotide sequences which hybridize under stringent conditions to a nucleic acid complementary to one of SEQ ID NOs. 8-3795, nucleic acids comprising nucleotide sequences which hybridize under stringent conditions to a fragment comprising at least 10, 15, 20, 25, 30, 35, 40, 50, 75, 100, 150, 200, 300, 400, or 500 consecutive nucleotides of the sequence complementary to one of SEQ ID NOs. 8-3795, nucleic acids comprising nucleotide sequences which hybridize under stringent conditions to a nucleic acid selected from the group consisting of SEQ ID NOS.: 3796-3800, 3806-4860, 5916-10012, nucleic acids comprising nucleotide sequences which hybridize under stringent conditions to a fragment comprising at least 10, 15, 20, 25, 30, 35, 40, 50, 75, 100, 150, 200, 300, 400, or 500 consecutive nucleotides of one of SEQ ID NOS.: 3796-3800, 3806-4860, 5916-10012, nucleic acids comprising nucleotide sequences which hybridize under stringent conditions to a nucleic acid complementary to one of SEQ ID NOS.: 3796-3800, 3806-4860, 5916-10012, nucleic acids comprising nucleotide sequences which hybridize under stringent conditions to a fragment comprising at least 10, 15, 20, 25, 30, 35, 40, 50, 75, 100, 150, 200, 300,400, or 500 consecutive nucleotides of the sequence complementary to one of SEQ ID NOS.: 3796-3800, 3806-4860, 5916-10012, nucleic acids comprising nucleotide sequences which hybridize under stringent conditions to a nucleic acid selected from the group consisting of SEQ ID NOS.: 3796-3800, 3806-4860, 5916-10012, and nucleic acids comprising nucleotide sequences which hybridize under stringent conditions to a fragment comprising at least 10, 15, 20, 25, 30, 35, 40, 50, 75, 100, 150, 200, 300, 400, or 500 consecutive nucleotides of one of SEQ ID NOS.: 3796-3800, 3806-4860, 5916-10012.

The libraries may also be screened to identify homologous nucleic coding nucleic acids or homologous antisense nucleic acids comprising nucleotide sequences which hybridize under moderate conditions to a nucleic acid selected from the group consisting of SEQ ID NOs.: 8-3795, nucleic acids comprising nucleotide sequences which hybridize under moderate conditions to a fragment comprising at least 10, 15, 20, 25, 30, 35, 40, 50, 75, 100, 150, 200, 300, 400, or 500 consecutive nucleotides of one of SEQ ID NOs. 8-3795, nucleic acids comprising nucleotide sequences which hybridize under moderate conditions to a nucleic acid complementary to one of SEQ ID NOs. 8-3795, nucleic acids comprising nucleotide sequences which hybridize under moderate conditions to a fragment comprising at least 10, 15, 20, 25, 30, 35, 40, 50, 75, 100, 150, 200, 300, 400, or 500 consecutive nucleotides of the sequence complementary to one of SEQ ID NOs. 8-3795, nucleic acids comprising nucleotide sequences which hybridize under moderate conditions to a nucleic acid selected from the group consisting of SEQ ID NOS.: 3796-3800, 3806-4860, 5916-10012, nucleic acids comprising nucleic acid sequences which hybridize under moderate conditions to a fragment comprising at least 10, 15, 20, 25, 30, 35, 40, 50, 75, 100, 150,200, 300, 400, or 500 consecutive nucleotides of one of SEQ ID NOS.: 3796-3800, 3806-4860, 5916-10012, nucleic acids comprising nucleotide sequences which hybridize under moderate conditions to a nucleic acid complementary to one of SEQ ID NOS.: 3796-3800, 3806-4860, 5916-10012 and nucleic acids comprising nucleotide sequences which hybridize under moderate conditions to a fragment comprising at least 10, 15, 20,25, 30, 35, 40, 50, 75, 100, 150, 200, 300, 400, or 500 consecutive nucleotides of the sequence complementary to one of SEQ ID NOS.: 3796-3800, 3806-4860, 5916-10012.

The homologous nucleic coding nucleic acids, homologous antisense nucleic acids or homologous polypeptides identified as above can then be used as targets or tools for the identification of new, antimicrobial compounds using methods such as those described herein. In some embodiments, the homologous coding nucleic acids, homologous antisense nucleic acids, or homologous polypeptides may be used to identify compounds with activity against more than one microorganism.

For example, the preceding methods may be used to isolate homologous coding nucleic acids or homologous antisense nucleic acids comprising a nucleotide sequence with at least 97%, at least 95%, at least 90%, at least 85%, at least 80%, or at least 70% nucleotide sequence identity to a nucleotide sequence selected from the group consisting of one of the sequences of SEQ ID NOS. 8-3795, fragments comprising at least 10, 15, 20, 25, 30, 35, 40, 50, 75, 100, 150, 200, 300, 400, or 500 consecutive nucleotides thereof, and the sequences complementary thereto. The preceding methods may also be used to isolate homologous coding nucleic acids or homologous antisense nucleic acids comprising a nucleotide sequence with at least 97%, at least 95%, at least 90%, at least 85%, at least 80%, or at least 70% nucleotide sequence identity to a nucleotide sequence selected from the group consisting of one of the nucleotide sequences of SEQ ID NOS.: 3796-3800, 3806-4860, 5916-10012, fragments comprising at least 10, 15, 20, 25, 30, 35, 40, 50, 75, 100, 150, 200, 300, 400, or 500 consecutive nucleotides thereof, and the sequences complementary thereto. In some embodiments, the preceding methods may be used to isolate homologous coding nucleic acids or homologous antisense nucleic acids comprising a nucleotide sequence with at least 97%, at least 95%, at least 90%, at least 85%, at least 80%, or at least 70% nucleotide sequence identity to a nucleic acid sequence selected from the group consisting of one of the sequences of SEQ ID NOS. 3796-3800, 3806-4860, 5916-10012, fragments comprising at least 10, 15, 20,25, 30, 35, 40, 50, 75, 100, 150, 200, 300, 400, or 500 consecutive nucleotides thereof, and the sequences complementary thereto. Identity may be measured using BLASTN version 2.0 with the default parameters. (Altschul, S. F. et al. Gapped BLAST and PSI-BLAST: A New Generation of Protein Database Search Programs, Nucleic Acid Res. 25: 3389-3402 (1997), the disclosure of which is incorporated herein by reference in its entirety). For example, the homologous polynucleotides may comprise a coding sequence which is a naturally occurring allelic variant of one of the coding sequences described herein. Such allelic variants may have a substitution, deletion or addition of one or more nucleotides when compared to the nucleic acids of SEQ ID NOs: 8-3795, SEQ ID NOS.: 3796-3800, 3806-4860, 5916-10012 or the nucleotide sequences complementary thereto.

Additionally, the above procedures may be used to isolate homologous coding nucleic acids which encode polypeptides having at least 99%, 95%, at least 90%, at least 85%, at least 80%, at least 70%, at least 60%, at least 50%, at least 40% or at least 25% amino acid identity or similarity to a polypeptide comprising the sequence of one of SEQ ID NOs: 3801-3805, 4861-5915, 10013-14110 or to a polypeptpide whose expression is inhibited by a nucleic acid of one of SEQ ID NOs: 8-3795 or fragments comprising at least 5, 10, 15, 20,25, 30, 35, 40, 50, 75, 100, or 150 consecutive amino acids thereof as determined using the FASTA version 3.0t78 algorithm with the default parameters. Alternatively, protein identity or similarity may be identified using BLASTP with the default parameters, BLASTX with the default parameters, or TBLASTN with the default parameters. (Altschul, S. F. et al. Gapped BLAST and PSI-BLAST: A New Generation of Protein Database Search Programs, Nucleic Acid Res. 25: 3389-3402 (1997), the disclosure of which is incorporated herein by reference in its entirety).

Alternatively, homologous coding nucleic acids, homologous antisense nucleic acids or homologous polypeptides may be identified by searching a database to identify sequences having a desired level of nucleotide or amino acid sequence homology to a nucleic acid or polypeptide involved in proliferation or an antisense nucleic acid to a nucleic acid involved in microbial proliferation. A variety of such databases are available to those skilled in the art, including GenBank and GenSeq. In some embodiments, the databases are screened to identify nucleic acids with at least 97%, at least 95%, at least 90%, at least 85%, at least 80%, or at least 70% nucleotide sequence identity to a nucleic acid required for proliferation, an antisense nucleic acid which inhibits proliferation, or a portion of a nucleic acid required for proliferation or a portion of an antisense nucleic acid which inhibits proliferation. For example, homologous coding sequences may be identified by using a database to identify nucleic acids homologous to one of SEQ ID Nos. 8-3795, homologous to fragments comprising at least 10, 15, 20, 25, 30, 35, 40, 50, 75, 100, 150, 200, 300, 400, or 500 consecutive nucleotides thereof, nucleic acids homologous to one of SEQ ID NOS.: 3796-3800, 3806-4860, 5916-10012, homologous to fragments comprising at least 10, 15, 20, 25, 30, 35,40, 50, 75, 100, 150, 200, 300,400, or 500 consecutive nucleotides of one of SEQ ID NOS.: 3796-3800, 3806-4860, 5916-10012, nucleic acids homologous to one of SEQ ID Nos. 8-3795, homologous to fragments comprising at least 10, 15, 20,25,30, 35, 40, 50, 75, 100, 150,200, 300, 400, or 500 consecutive nucleotides thereof or nucleic acids homologous to the sequences complementary to any of the preceding nucleic acids. In other embodiments, the databases are screened to identify polypeptides having at least 99%, 95%, at least 90%, at least 85%, at least 80%, at least 70%, at least 60%, at least 50%, at least 40% or at least 25% amino acid sequence identity or similarity to a polypeptide involved in proliferation or a portion thereof. For example, the database may be screened to identify polypeptides homologous to a polypeptide comprising one of SEQ ID NOs: 3801-3805, 4861-5915, 10013-14110, a polypeptide whose expression is inhibited by a nucleic acid of one of SEQ ID NOs: 8-3795 or homologous to fragments comprising at least 5, 10, 15, 20, 25, 30, 35, 40, 50, 75, 100, or 150 consecutive amino acids of any of the preceding polypeptides. In some embodiments, the database may be screened to identify homologous coding nucleic acids, homologous antisense nucleic acids or homologous polypeptides from cells or microorganisms other than the Staphylococcus aureus, Salmonella typhimurium, Klebsiella pneumoniae, Pseudomonas aeruginosa and Enterococcus faecalis, Escherichia coli, Enterococcus faecalis, Haemophilus influenzae, Helicobacter pylori, Klebsiella pneumoniae, Pseudomonas aeruginosa, Staphylococcus aureus, or Salmonella typhi species from which they were obtained. For example the database may be screened to identify homologous coding nucleic acids, homologous antisense nucleic acids or homologous polypeptides from microorganisms such as Anaplasma marginale, Aspergillus fumigatus, Bacillus anthracis, Bacterioides fragilis Bordetella pertussis, Burkholderia cepacia, Campylobacter jejuni, Candida albicans, Candida glabrata (also called Torulopsis glabrata), Candida tropicalis, Candida parapsilosis, Candida guilliermondii, Candida krusei, Candida kefyr (also called Candida pseudotropicalis), Candida dubliniensis, Chlamydia pneumoniae, Chlamydia trachomatus, Clostridium botulinum, Clostridium difficile, Clostridium perfringens, Coccidiodes immitis, Corynebacterium diptheriae, Cryptococcus neoformans, Enterobacter cloacae, Enterococcus faecalis, Enterococcus faecium, Escherichia coli, Haemophilus influenzae, Helicobacter pylori, Histoplasma capsulatum, Klebsiella pneumoniae, Listeria monocytogenes, Mycobacterium leprae, Mycobacterium tuberculosis, Neisseria gonorrhoeae, Neisseria meningitidis, Nocardia asteroides, Pasteurella haemolytica, Pasteurella multocida, Pneumocystis carinii, Proteus vulgaris, Pseudomonas aeruginosa, Salmonella bongori, Salmonella cholerasuis, Salmonella enterica, Salmonella paratyphi, Salmonella typhi, Salmonella typhimurium, Staphylococcus aureus, Listeria monocytogenes, Moxarella catarrhalis, Shigella boydii, Shigella dysenteriae, Shigella flexneri, Shigella sonnei, Staphylococcus epidermidis, Streptococcus pneumoniae, Streptococcus mutans, Treponema pallidum, Yersinia enterocolitica, Yersinia pestis or any species falling within the genera of any of the above species, including coagulase negative Staphylococcus. In some embodiments, the homologous coding nucleic acids, homologous antisense nucleic acids, or homologous polypeptides are from an organism other than E. coli.

In another embodiment, gene expression arrays and microarrays can be employed. Gene expression arrays are high density arrays of DNA samples deposited at specific locations on a glass chip, nylon membrane, or the like. Such arrays can be used by researchers to quantify relative gene expression under different conditions. Gene expression arrays are used by researchers to help identify optimal drug targets, profile new compounds, and determine disease pathways. An example of this technology is found in U.S. Pat. No. 5,807,522, which is hereby incorporated by reference.

It is possible to study the expression of all genes in the genome of a particular microbial organism using a single array. For example, the arrays may consist of 12×24 cm nylon filters containing PCR products corresponding to ORFs from Staphylococcus aureus, Salmonella typhimurium, Klebsiella pneumoniae, Pseudomonas aeruginosa and Enterococcus faecalis, Escherichia coli Enterococcus faecalis, Haemophilus influenzae, Helicobacter pylori, Klebsiella pneumoniae, Pseudomonas aeruginosa, Staphylococcus aureus, or Salmonella typhi (including the nucleic acids of SEQ ID NOs.: 3796-3800, 3806-4860, 5916-10012). 10 ngs of each PCR product are spotted every 1.5 mm on the filter. Single stranded labeled cDNAs are prepared for hybridization to the array (no second strand synthesis or amplification step is done) and placed in contact with the filter. Thus the labeled cDNAs are of “antisense” orientation. Quantitative analysis is done by phosphorimager.

Hybridization of cDNA made from a sample of total cell mRNA to such an array followed by detection of binding by one or more of various techniques known to those in the art results in a signal at each location on the array to which cDNA hybridized. The intensity of the hybridization signal obtained at each location in the array thus reflects the amount of mRNA for that specific gene that was present in the sample. Comparing the results obtained for mRNA isolated from cells grown under different conditions thus allows for a comparison of the relative amount of expression of each individual gene during growth under the different conditions.

Gene expression arrays may be used to analyze the total mRNA expression pattern at various time points after induction of an antisense nucleic acid complementary to a proliferation-required gene. Analysis of the expression pattern indicated by hybridization to the array provides information on other genes whose expression is influenced by antisense expression. For example, if the antisense is complementary to a gene for ribosomal protein L7/L12 in the 50S subunit, levels of other mRNAs may be observed to increase, decrease or stay the same following expression of antisense to the L7/L12 gene. If the antisense is complementary to a different 50S subunit ribosomal protein mRNA (e.g. L25), a different mRNA expression pattern may result. Thus, the mRNA expression pattern observed following expression of an antisense nucleic acid comprising a nucleotide sequence complementary to a proliferation required gene may identify other proliferation-required nucleic acids. In addition, the mRNA expression patterns observed when the bacteria are exposed to candidate drug compounds or known antibiotics may be compared to those observed with antisense nucleic acids comprising a nucleotide sequence complementary to a proliferation-required nucleic acid. If the mRNA expression pattern observed with the candidate drug compound is similar to that observed with the antisense nucleic acid, the drug compound may be a promising therapeutic candidate. Thus, the assay would be useful in assisting in the selection of promising candidate drug compounds for use in drug development.

In cases where the source of nucleic acid deposited on the array and the source of the nucleic acid being hybridized to the array are from two different cells or microorganisms, gene expression arrays can identify homologous nucleic acids in the two cells or microorganisms.

The present invention also contemplates additional methods for screening other microorganisms for proliferation-required genes. In one aspect of this embodiment, an antisense nucleic acid comprising a nucleotide sequence complementary to the proliferation-required sequences from Staphylococcus aureus, Salmonella typhimurium, Klebsiella pneumoniae, Pseudomonas aeruginosa and Enterococcus faecalis, Escherichia coli, Enterococcus faecalis, Haemophilus influenzae, Helicobacter pylori, Klebsiella pneumoniae, Pseudomonas aeruginosa, Staphylococcus aureus, or Salmonella typhi or a portion thereof is transcribed in an antisense orientation in such a way as to alter the level or activity of a nucleic acid required for proliferation of an autologous or heterologous cell or microorganism. For example, the antisense nucleic acid may be a homologous antisense nucleic acid such as an antisense nucleic acid homologous to the nucleotide sequence complementary to one of SEQ ID NOs.: 3796-3800, 3806-4860, 5916-10012, an antisense nucleic acid comprising a nucleotide sequence homologous to one of SEQ ID Nos.: 8-3795, or an antisense nucleic acid comprising a nucleotide sequence complementary to a portion of any of the preceding nucleic acids. The cell or microorganism transcribing the homologous antisense nucleic acid may be used in a cell-based assay, such as those described herein, to identify candidate antibiotic compounds. In another embodiment, the conserved portions of nucleotide sequences identified as proliferation-required can be used to generate degenerate primers for use in the polymerase chain reaction (PCR). The PCR technique is well known in the art. The successful production of a PCR product using degenerate probes generated from the nucleotide sequences identified herein indicates the presence of a homologous gene sequence in the species being screened. This homologous gene is then isolated, expressed, and used as a target for candidate antibiotic compounds. In another aspect of this embodiment, the homologous gene (for example a homologous coding nucleic acid )thus identified, or a portion thereof, is transcribed in an autologous cell or microorganism or in a heterologous cell or microorganism in an antisense orientation in such a way as to alter the level or activity of a homologous gene required for proliferation in the autologous or heterologous cell or microorganism. Alternatively, a homologous antisense nucleic acid may be transcribed in an autologous or heterologous cell or microorganism in such a way as to alter the level or activity of a gene product required for proliferation in the autologous or heterologous cell or microorganism.

The nucleic acids homologous to the genes required for the proliferation of Staphylococcus aureus, Salmonella typhimurium, Klebsiella pneumoniae, Pseudomonas aeruginosa and Enterococcus faecalis, Escherichia coli, Enterococcus faecalis, Haemophilus influenzae, Helicobacter pylori, Klebsiella pneumoniae, Pseudomonas aeruginosa, Staphylococcus aureus, or Salmonella typhi or the sequences complementary thereto may be used to identify homologous coding nucleic acids or homologous antisense nucleic acids from cells or microorganisms other than Staphylococcus aureus, Salmonella typhimurium, Klebsiella pneumoniae, Pseudomonas aeruginosa and Enterococcus faecalis, Escherichia coli, Enterococcus faecalis, Haemophilus influenzae, Helicobacter pylori, Klebsiella pneumoniae, Pseudomonas aeruginosa, Staphylococcus aureus, or Salmonella typhi to inhibit the proliferation of cells or microorganisms other than Staphylococcus aureus, Salmonella typhimurium, Klebsiella pneumoniae, Pseudomonas aeruginosa and Enterococcus faecalis, Escherichia coli, Enterococcus faecalis, Haemophilus influenzae, Helicobacter pylori, Klebsiella pneumoniae, Pseudomonas aeruginosa, Staphylococcus aureus, or Salmonella typhi by inhibiting the activity or reducing the amount of the identified homologous coding nucleic acid or homologous polypeptide in the cell or microorganism other than Staphylococcus aureus, Salmonella typhimurium, Klebsiella pneumoniae, Pseudomonas aeruginosa Enterococcus faecalis, Escherichia coli, Enterococcus faecalis, Haemophilus influenzae, Helicobacter pylori, or Salmonella typhi or to identify compounds which inhibit the growth of cells or microorganisms other than Staphylococcus aureus, Salmonella typhimurium, Klebsiella pneumoniae, Pseudomonas aeruginosa, Enterococcus faecalis, Escherichia coli, Enterococcus faecalis, Haemophilus influenzae, Helicobacter pylori, or Salmonella typhi as described below. For example, the nucleic acids homologous to proliferation-required genes from Staphylococcus aureus, Salmonella typhimurium, Klebsiella pneumoniae, Pseudomonas aeruginosa and Enterococcus faecalis, Escherichia coli, Enterococcus faecalis, Haemophilus influenzae, Helicobacter pylori, Klebsiella pneumoniae, Pseudomonas aeruginosa, Staphylococcus aureus, or Salmonella typhi or the sequences complementary thereto may be used to identify compounds which inhibit the growth of Anaplasma marginale, Aspergillus fumigatus, Bacillus anthracis, Bacterioides fragilis Bordetella pertussis, Burkholderia cepacia, Campylobacter jejuni, Candida albicans, Candida glabrata (also called Torulopsis glabrata), Candida tropicalis, Candida parapsilosis, Candida guilliermondii, Candida krusei, Candida kefyr (also called Candida pseudotropicalis), Candida dubliniensis, Chlamydia pneumoniae, Chlamydia trachomatus, Clostridium botulinum, Clostridium difficile, Clostridium perfringens, Coccidiodes immitis, Corynebacterium diptheriae, Cryptococcus neoformans, Enterobacter cloacae, Enterococcus faecalis, Enterococcus faecium, Escherichia coli, Haemophilus influenzae, Helicobacter pylori, Histoplasma capsulatum, Klebsiella pneumoniae, Listeria monocytogenes, Mycobacterium leprae, Mycobacterium tuberculosis, Neisseria gonorrhoeae, Neisseria meningitidis, Nocardia asteroides, Pasteurella haemolytica, Pasteurella multocida, Pneumocystis carinii, Proteus vulgaris, Pseudomonas aeruginosa, Salmonella bongori, Salmonella cholerasuis, Salmonella enterica, Salmonella paratyphi, Salmonella typhi, Salmonella typhimurium, Staphylococcus aureus, Listeria monocytogenes, Moxarella catarrhalis, Shigella boydii, Shigella dysenteriae, Shigella flexneri, Shigella sonnei, Staphylococcus epidermidis, Streptococcus pneumoniae, Streptococcus mutans, Treponema pallidum, Yersinia enterocolitica, Yersinia pestis and any species falling within the genera of any of the above species. In some embodiments of the present invention, the nucleic acids homologous to proliferation-required sequences from Staphylococcus aureus, Salmonella typhimurium, Klebsiella pneumoniae, Pseudomonas aeruginosa and Enterococcus faecalis, Escherichia coli, Enterococcus faecalis, Haemophilus influenzae, Helicobacter pylori, Klebsiella pneumoniae, Pseudomonas aeruginosa, Staphylococcus aureus, or Salmonella typhi (including nucleic acids homologous to one of SEQ ID NOs.: 3796-3800, 3806-4860, 5916-10012) or the sequences complementary thereto (including nucleic acids homologous to one of SEQ ID NOs.: 8-3795) are used to identify proliferation-required sequences in an organism other than E. coli.

In another embodiment of the present invention, antisense nucleic acids complementary to the sequences identified as required for proliferation or portions thereof (including antisense nucleic acids comprising a nucleotide sequence complementary to one of SEQ ID NOs.: 3796-3800, 3806-4860, 5916-10012 or portions thereof, such as the nucleic acids of SEQ ID NOs.: 8-3795) are transferred to vectors capable of function within a species other than the species from which the sequences were obtained. For example, the vector may be functional in Anaplasma marginale, Aspergillus fumigatus, Bacillus anthracis, Bacterioides fragilis Bordetella pertussis, Burkholderia cepacia, Campylobacter jejuni, Candida albicans, Candida glabrata (also called Torulopsis glabrata), Candida tropicalis, Candida parapsilosis, Candida guilliermondii, Candida krusei, Candida kefyr (also called Candida pseudotropicalis), Candida dubliniensis, Chlamydia pneumoniae, Chlamydia trachomatus, Clostridium botulinum, Clostridium difficile, Clostridium perfringens, Coccidiodes immitis, Corynebacterium diptheriae, Cryptococcus neoformans, Enterobacter cloacae, Enterococcus faecalis, Enterococcus faecium, Escherichia coli, Haemophilus influenzae, Helicobacter pylori, Histoplasma capsulatum, Klebsiella pneumoniae, Listeria monocytogenes, Mycobacterium leprae, Mycobacterium tuberculosis, Neisseria gonorrhoeae, Neisseria meningitidis, Nocardia asteroides, Pasteurella haemolytica, Pasteurella multocida, Pneumocystis carinii, Proteus vulgaris, Pseudomonas aeruginosa, Salmonella bongori, Salmonella cholerasuis, Salmonella enterica, Salmonella paratyphi, Salmonella typhi, Salmonella typhimurium, Staphylococcus aureus, Listeria monocytogenes, Moxarella catarrhalis, Shigella boydii, Shigella dysenteriae, Shigella flexneri, Shigella sonnei, Staphylococcus epidermidis, Streptococcus pneumoniae, Streptococcus mutans, Treponema pallidum, Yersinia enterocolitica, Yersinia pestis or any species falling within the genera of any of the above species. In some embodiments of the present invention, the vector may be functional in an organism other than E. coli. As would be appreciated by one of ordinary skill in the art, vectors may contain certain elements that are species specific. These elements can include promoter sequences, operator sequences, repressor genes, origins of replication, ribosomal binding sequences, termination sequences, and others. To use the antisense nucleic acids, one of ordinary skill in the art would know to use standard molecular biology techniques to isolate vectors containing the sequences of interest from cultured bacterial cells, isolate and purify those sequences, and subclone those sequences into a vector adapted for use in the species of bacteria to be screened.

Vectors for a variety of other species are known in the art. For example, numerous vectors which function in E. coli are known in the art. Also, Pla et al. have reported an expression vector that is functional in a number of relevant hosts including: Salmonella typhimurium, Pseudomonas putida, and Pseudomonas aeruginosa. J. Bacteriol. 172(8):4448-55 (1990). Brunschwig and Darzins (Gene (1992) 111:35-4, the disclosure of which is incorporated herein by reference in its entirety) described a shuttle expression vector for Pseudomonas aeruginosa. Similarly many examples exist of expression vectors that are freely transferable among various Gram-positive microorganisms. Expression vectors for Enterococcus faecalis may be engineered by incorporating suitable promoters into a pAK80 backbone (Israelsen, H., S. M. Madsen, A. Vrang, E. B. Hansen and E. Johansen. 1995. Appl. Environ. Microbiol. 61:2540-2547, the disclosure of which is incorporated herein by reference in its entirety).

Following the subcloning of the antisense nucleic acids complementary to proliferation-required sequences from Staphylococcus aureus, Salmonella typhimurium, Klebsiella pneumoniae, Pseudomonas aeruginosa and Enterococcus faecalis, Escherichia coli, Enterococcus faecalis, Haemophilus influenzae, Helicobacter pylori, Klebsiella pneumoniae, Pseudomonas aeruginosa, Staphylococcus aureus, or Salmonella typhi or portions thereof into a vector functional in a second cell or microorganism of interest (i.e. a cell or microorganism other than the one from which the identified nucleic acids were obtained), the antisense nucleic acids are conditionally transcribed to test for bacterial growth inhibition. The nucleotide sequences of the nucleic acids from Staphylococcus aureus, Salmonella typhimurium, Klebsiella pneumoniae, Pseudomonas aeruginosa, Enterococcus faecalis, Escherichia coli Enterococcus faecalis, Haemophilus influenzae, Helicobacter pylori, or Salmonella typhi that, when transcribed, inhibit growth of the second cell or microorganism are compared to the known genomic sequence of the second cell or microorganism to identify the homologous gene from the second organism. If the homologous sequence from the second cell or microorganism is not known, it may be identified and isolated by hybridization to the proliferation-required Staphylococcus aureus, Salmonella typhimurium, Klebsiella pneumoniae, Pseudomonas aeruginosa, Enterococcus faecalis Escherichia coli, Enterococcus faecalis, Haemophilus influenzae, Helicobacter pylori, or Salmonella typhi sequence of interest or by amplification using PCR primers based on the proliferation-required nucleotide sequence of interest as described above. In this way, sequences which may be required for the proliferation of the second cell or microorganism may be identified. For example, the second microorganism may be Anaplasma marginale, Aspergillus fumigatus, Bacillus anthracis, Bacterioides fragilis Bordetella pertussis, Burkholderia cepacia, Campylobacter jejuni, Candida albicans, Candida glabrata (also called Torulopsis glabrata), Candida tropicalis, Candida parapsilosis, Candida guilliermondii, Candida krusei, Candida kefyr (also called Candida pseudotropicalis), Candida dubliniensis, Chlamydia pneumoniae, Chlamydia trachomatus, Clostridium botulinum, Clostridium difficile, Clostridium perfringens, Coccidiodes immitis, Corynebacterium diptheriae, Cryptococcus neoformans, Enterobacter cloacae, Enterococcus faecalis, Enterococcus faecium, Escherichia coli, Haemophilus influenzae, Helicobacter pylori, Histoplasma capsulatum, Klebsiella pneumoniae, Listeria monocytogenes, Mycobacterium leprae, Mycobacterium tuberculosis, Neisseria gonorrhoeae, Neisseria meningitidis, Nocardia asteroides, Pasteurella haemolytica, Pasteurella multocida, Pneumocystis carinii, Proteus vulgaris, Pseudomonas aeruginosa, Salmonella bongori, Salmonella cholerasuis, Salmonella enterica, Salmonella paratyphi, Salmonella typhi, Salmonella typhimurium, Staphylococcus aureus, Listeria monocytogenes, Moxarella catarrhalis, Shigella boydii, Shigella dysenteriae, Shigella flexneri, Shigella sonnei, Staphylococcus epidermidis, Streptococcus pneumoniae, Streptococcus mutans, Treponema pallidum, Yersinia enterocolitica, Yersinia pestis or any species falling within the genera of any of the above species. In some embodiments of the present invention, the second microorganism is an organism other than E. coli.

The homologous nucleic acid sequences from the second cell or microorganism which are identified as described above may then be operably linked to a promoter, such as an inducible promoter, in an antisense orientation and introduced into the second cell or microorganism. The techniques described herein for identifying Staphylococcus aureus, Salmonella typhimurium, Klebsiella pneumoniae, Pseudomonas aeruginosa and Enterococcus faecalis, Escherichia coli, Enterococcus faecalis, Haemophilus influenzae, Helicobacter pylori, Klebsiella pneumoniae, Pseudomonas aeruginosa, Staphylococcus aureus, or Salmonella typhi genes required for proliferation may thus be employed to determine whether the identified nucleotide sequences from a second cell or microorganism inhibit the proliferation of the second cell or microorganism. For example, the second microorganism may be Anaplasma marginale, Aspergillus fumigatus, Bacillus anthracis, Bacterioides fragilis Bordetella pertussis, Burkholderia cepacia, Campylobacter jejuni, Candida albicans, Candida glabrata (also called Torulopsis glabrata), Candida tropicalis, Candida parapsilosis, Candida guilliermondii, Candida krusei, Candida kefyr (also called Candida pseudotropicalis), Candida dubliniensis, Chlamydia pneumoniae, Chlamydia trachomatus, Clostridium botulinum, Clostridium difficile, Clostridium perfringens, Coccidiodes immitis, Corynebacterium diptheriae, Cryptococcus neoformans, Enterobacter cloacae, Enterococcus faecalis, Enterococcus faecium, Escherichia coli, Haemophilus influenzae, Helicobacter pylori, Histoplasma capsulatum, Klebsiella pneumoniae, Listeria monocytogenes, Mycobacterium leprae, Mycobacterium tuberculosis, Neisseria gonorrhoeae, Neisseria meningitidis, Nocardia asteroides, Pasteurella haemolytica, Pasteurella multocida, Pneumocystis carinii, Proteus vulgaris, Pseudomonas aeruginosa, Salmonella bongori, Salmonella cholerasuis, Salmonella enterica, Salmonella paratyphi, Salmonella typhi, Salmonella typhimurium, Staphylococcus aureus, Listeria monocytogenes, Moxarella catarrhalis, Shigella boydii, Shigella dysenteriae, Shigella flexneri, Shigella sonnei, Staphylococcus epidermidis, Streptococcus pneumoniae, Streptococcus mutans, Treponema pallidum, Yersinia enterocolitica, Yersinia pestis or any species falling within the genera of any of the above species. In some embodiments of the present invention, the second microorganism may be an organism other than E. coli.

Antisense nucleic acids required for the proliferation of microorganisms other than Staphylococcus aureus, Salmonella typhimurium, Klebsiella pneumoniae, Pseudomonas aeruginosa, Enterococcus faecalis, Escherichia coli, Enterococcus faecalis, Haemophilus influenzae, Helicobacter pylori, or Salmonella typhi or the genes corresponding thereto, may also be hybridized to a microarray containing the Staphylococcus aureus, Salmonella typhimurium, Klebsiella pneumoniae, Pseudomonas aeruginosa, Enterococcus faecalis ORFs, Escherichia coli, Enterococcus faecalis, Haemophilus influenzae, Helicobacter pylori, and Salmonella typhi (including the nucleic acids of SEQ ID NOs.: 3796-3800, 3806-4860, 5916-10012) to gauge the homology between the Staphylococcus aureus, Salmonella typhimurium, Klebsiella pneumoniae, Pseudomonas aeruginosa, Enterococcus faecalis, Escherichia coli, Enterococcus faecalis, Haemophilus influenzae, Helicobacter pylori, or Salmonella typhi sequences and the proliferation-required nucleic acids from other cells or microorganisms. For example, the proliferation-required nucleic acid may be from Anaplasma marginale, Aspergillus fumigatus, Bacillus anthracis, Bacterioides fragilis Bordetella pertussis, Burkholderia cepacia, Campylobacter jejuni, Candida albicans, Candida glabrata (also called Torulopsis glabrata), Candida tropicalis, Candida parapsilosis, Candida guilliermondii, Candida krusei, Candida kefyr (also called Candida pseudotropicalis), Candida dubliniensis, Chlamydia pneumoniae, Chlamydia trachomatus, Clostridium botulinum, Clostridium difficile, Clostridium perfringens, Coccidiodes immitis, Corynebacterium diptheriae, Cryptococcus neoformans, Enterobacter cloacae, Enterococcus faecalis, Enterococcus faecium, Escherichia coli, Haemophilus influenzae, Helicobacter pylori, Histoplasma capsulatum, Klebsiella pneumoniae, Listeria monocytogenes, Mycobacterium leprae, Mycobacterium tuberculosis, Neisseria gonorrhoeae, Neisseria meningitidis, Nocardia asteroides, Pasteurella haemolytica, Pasteurella multocida, Pneumocystis carinii, Proteus vulgaris, Pseudomonas aeruginosa, Salmonella bongori, Salmonella cholerasuis, Salmonella enterica, Salmonella paratyphi, Salmonella typhi, Salmonella typhimurium, Staphylococcus aureus, Listeria monocytogenes, Moxarella catarrhalis, Shigella boydii, Shigella dysenteriae, Shigella flexneri, Shigella sonnei, Staphylococcus epidermidis, Streptococcus pneumoniae, Streptococcus mutans, Treponema pallidum, Yersinia enterocolitica, Yersinia pestis or any species falling within the genera of any of the above species. In some embodiments of the present invention, the proliferation-required nucleotide sequences from Staphylococcus aureus, Salmonella typhimurium, Klebsiella pneumoniae, Pseudomonas aeruginosa, Enterococcus faecalis, Escherichia coli, Enterococcus faecalis, Haemophilus influenzae, Helicobacter pylori, Salmonella typhi or homologous nucleic acids are used to identify proliferation-required sequences in an organism other than E. coli. In some embodiments of the present invention, the proliferation-required sequences may be from an organism other than E. coli. The proliferation-required nucleic acids from a cell or microorganism other than Staphylococcus aureus, Salmonella typhimurium, Klebsiella pneumoniae, Pseudomonas aeruginosa, Enterococcus faecalis, Escherichia coli, Enterococcus faecalis, Haemophilus influenzae, Helicobacter pylori, or Salmonella typhi may be hybridized to the array under a variety of conditions which permit hybridization to occur when the probe has different levels of homology to the nucleotide sequence on the microarray. This would provide an indication of homology across the cells or microorganisms as well as clues to other possible essential genes in these cells or microorganisms.

In still another embodiment, the antisense nucleic acids of the present invention (including the antisense nucelic acids of SEQ ID NOs. 8-3795 or homologous antisense nucleic acids) that inhibit bacterial growth or proliferation can be used as antisense therapeutics for killing bacteria. The antisense sequences can be complementary to one of SEQ ID NOs.: 3796-3800, 3806-4860, 5916-10012, homologous nucleic acids, or portions thereof. Alternatively, antisense therapeutics can be complementary to operons in which proliferation-required genes reside (i.e. the antisense nucleic acid may hybridize to a nucleotide sequence of any gene in the operon in which the proliferation-required genes reside). Further, antisense therapeutics can be complementary to a proliferation-required gene or portion thereof with or without adjacent noncoding sequences, an intragenic sequence (i.e. a sequence within a gene), an intergenic sequence (i.e. a sequence between genes), a sequence spanning at least a portion of two or more genes, a 5′ noncoding region or a 3′ noncoding region located upstream or downstream from the actual sequence that is required for bacterial proliferation or an operon containing a proliferation-required gene.

In addition to therapeutic applications, the present invention encompasses the use of nucleic acids complementary to nucleic acids required for proliferation as diagnostic tools. For example, nucleic acid probes comprising nucleotide sequences complementary to proliferation-required sequences that are specific for particular species of cells or microorganisms can be used as probes to identify particular microorganism species or cells in clinical specimens. This utility provides a rapid and dependable method by which to identify the causative agent or agents of a bacterial infection. This utility would provide clinicians the ability to accurately identify the species responsible for the infection and amdminister a compound effective against it. In an extension of this utility, antibodies generated against proteins translated from mRNA transcribed from proliferation-required sequences can also be used to screen for specific cells or microorganisms that produce such proteins in a species-specific manner.

Other embodiments of the present invention include methods of identifying compounds which inhibit the activity of gene products required for cellular proliferation using rational drug design. As discussed in more detail below, in such methods, the structure of the gene product is determined using techniques such as x-ray crystallography or computer modeling. Compounds are screened to identify those which have a structure which would allow them to interact with the gene product or a portion thereof to inhibit its activity. The compounds may be obtained using any of a variety of methods familiar to those skilled in the art, including combinatorial chemistry. In some embodiments, the compounds may be obtained from a natural product library. In some embodiments, compounds having a structure which allows them to interact with the active site of a gene product, such as the active site of an enzyme, or with a portion of the gene product which interacts with another biomolecule to form a complex are identified. If desired, lead compounds may be identified and further optimized to provide compounds which are highly effective against the gene product.

The following examples teach the genes of the present invention and a subset of uses for the genes identified as required for proliferation. These examples are illustrative only and are not intended to limit the scope of the present invention.

### EXAMPLES

The following examples are directed to the identification and exploitation of genes required for proliferation. Methods of gene identification are discussed as well as a variety of methods to utilize the identified sequences. It will be appreciated that any of the antisense nucleic acids, proliferartion-required genes or proliferation-required gene products described herein, or portions thereof, may be used in the procedures described below, including the antisense nucleic acids of SEQ ID NOs.: 8-3795, the nucleic acids of SEQ ID NOS.: 3796-3800, 3806-4860, 5916-10012, or the polypeptides of SEQ ID NOs.: 3801-3805, 4861-5915, 10013-14110. Likewise, homologous coding nucleic acids or portions thereof, may be used in any of the procedures described below.

Genes Identified as Required for Proliferation of Staphylococcus aureus, Salmonella typhimurium, Klebsiella pneumoniae, Pseudomonas aeruginosa or Enterococcus faecalis

Genomic fragments were operably linked to an inducible promoter in a vector and assayed for growth inhibition activity. Example 1 describes the examination of a library of genomic fragments cloned into vectors comprising inducible promoters. Upon induction with xylose or IPTG, the vectors produced an RNA molecule corresponding to the subcloned genomic fragments. In those instances where the genomic fragments were in an antisense orientation with respect to the promoter, the transcript produced was complementary to at least a portion of an MRNA (messenger RNA) encoding a Staphylococcus aureus, Salmonella typhimurium, Klebsiella pneumoniae, Pseudomonas aeruginosa or Enterococcus faecalis gene product such that they interacted with sense mRNA produced from various Staphylococcus aureus, Salmonella typhimurium, Klebsiella pneumoniae, Pseudomonas aeruginosa or Enterococcus faecalis genes and thereby decreased the translation efficiency or the level of the sense messenger RNA thus decreasing production of the protein encoded by these sense mRNA molecules. In cases where the sense mRNA encoded a protein required for proliferation, bacterial cells containing a vector from which transcription from the promoter had been induced failed to grow or grew at a substantially reduced rate. Additionally, in cases where the transcript produced was complementary to at least a portion of a non-translated RNA and where that non-translated RNA was required for proliferation, bacterial cells containing a vector from which transcription from the promoter had been induced also failed to grow or grew at a substantially reduced rate.

### Example 1

### Inhibition of Bacterial Proliferation after Induction of Antisense Expression

Nucleic acids involved in proliferation of Staphylococcus aureus, Salmonella typhimurium, and Klebsiella pneumoniae were identified as follows. Randomly generated fragments of Staphylococcus aureus, Salmonella typhimurium, Klebsiella pneumoniae, Pseudomonas aeruginosa or Enterococcus faecalis genomic DNA were transcribed from inducible promoters.

In the case of Staphylococcus aureus, a novel inducible promoter system, XylT5, comprising a modified T5 promoter fused to the xylO operater from the xyla promoter of Staphylococcus aureus was used. The promoter is described in U.S. Provisional Patent Application Ser. No. 60/259,434, the disclosure of which is incorporated herein by reference in its entirety. Transcription from this hybrid promoter is inducible by xylose.

Randomly generated fragments of Salmonella typhimurium genomic DNA were transcribed from an IPTG inducible promoter in pLEX5BA (Krause et al., J. Mol. Biol.

274: 365 (1997) or a derivative thereof. Randomly generated fragements of Klebsiella pneumoniae genomic DNA were expressed from an IPTG inducible promoter in pLEX5BA-Kan. To construct pLEX5BA-kan, pLEX5BA was digested to completion with ClaI in order to remove the bla gene. Then the plasmid was treated with a partial NotI digestion and blunted with T4 DNA polymerase. A 3.2 kbp fragment was then gel purified and ligated to a blunted 1.3 kbp kan gene from pKant. Kan resistant transformants were selected on Kan plates. Orientation of the kan gene was checked by SmaI digestion. A clone, which had the kan gene in the same orientation as the bla gene, was used to identify genes required for proliferation of Klebsiella pneumoniae.

Randomly generated fragments of Pseudomonas aeruginosa genomic DNA were trancribed from a two-component inducible promoter system. Integrated on the chromosome was the T7 RNA polymerase gene regulated by lacUV5/lacO (Brunschwig, E. and Darzins, A. 1992. Gene 1 11:35-41, the disclosure of which is incorporated herein by reference in its entirety). On a separate plasmid, a T7 gene 10 promoter, which is transcribed by T7 RNA polymerase, was fused with a lacO operator followed by a multiple cloning site.

Should the genomic DNA downstream of the promoter contain, in an antisense orientation, at least a portion of an MRNA or a non-translated RNA encoding a gene product involved in proliferation, then induction of transcription from the promoter will result in detectable inhibition of proliferation.

In the case of Staphylococcus aureus, a shotgun library of Staphylococcus aureus genomic fragments was cloned into the vector pXyIT5-P15a, which harbors the Xy1T5 inducible promoter. The vector was linearized at a unique BamHI site immediately downstream of the XyIT5 promoter/operator. The linearized vector was treated with shrimp alkaline phosphatase to prevent reclosure of the linearized ends. Genomic DNA isolated from Staphylococcus aureus strain RN450 was fully digested with the restriction enzyme Sau3A, or, alternatively, partially digested with DNase I and “blunt-ended” by incubating with T4 DNA polymerase. Random genomic fragments between 200 and 800 base pairs in length were selected by gel purification. The size-selected genomic fragments were added to the linearized and dephosphorylated vector at a molar ratio of 0.1 to 1, and ligated to form a shotgun library.

The ligated products were transformed into electrocompetent E. coli strain XL1-Blue MRF (Stratagene) and plated on LB medium with supplemented with carbenicillin at 100 μg/ml. Resulting colonies numbering 5×10⁵ or greater were scraped and combined, and were then subjected to plasmid purification.

The purified library was then transformed into electrocompetent Staphylococcus aureus RN4220. Resulting transformants were plated on agar containing LB+0.2% glucose (LBG medium)+chloramphenicol at 15 μg/ml (LBG+CM15 medium) in order to generate 100 to 150 platings at 500 colonies per plating. The colonies were subjected to robotic picking and arrayed into wells of 384 well culture dishes. Each well contained 100 μl of LBG+CM15 liquid medium. Inoculated 384 well dishes were incubated 16 hours at 37° C., and each well was robotically gridded onto solid LBG+CM15 medium with or without 2% xylose. Gridded plates were incubated 16 hours at 37° C., and then manually scored for arrayed colonies that were growth-compromised in the presence of xylose.

Arrayed colonies that were growth-sensitive on medium containing 2% xylose, yet were able to grow on similar medium lacking xylose, were subjected to further growth sensitivity analysis as follows: Colonies from the plate lacking xylose were manually picked and inoculated into individual wells of a 96 well culture dish containing LBG+CM15, and were incubated for 16 hours at 37° C. These cultures were robotically diluted 1/100 into fresh medium and allowed to incubate for 4 hours at 37° C., after which they were subjected to serial dilutions in a 384 well array and then gridded onto media containing 2% xylose or media lacking xylose. After growth for 16 hours at 37° C., the arrays that resulted on the two media were compared to each other. Clones that grew similarly at all dilutions on both media were scored as a negative and were no longer considered. Clones that grew on xylose medium but failed to grow at the same serial dilution on the non-xylose plate were given a score based on the differential, i.e. should the clone grow at a serial dilution of 10⁴ or less on the xylose plate and grow at a serial dilution of 10⁸ or less on the non-xylose plate, then the corresponding clone received a score of “4” representing the log difference in growth observed.

For Salmonella typhimurium and Klebsiella pneumoniae growth curves were carried out by back diluting cultures 1:200 into fresh media containing 1 mM IPTG or media lacking IPTG and measuring the OD₄₅₀ every 30 minutes (min). To study the effects of transcriptional induction on solid medium, 10², 10³, 10⁴, 10⁵, 10⁶, 10⁷ and 10⁸ fold dilutions of overnight cultures were prepared. Aliquots of from 0.5 to 3 μl of these dilutions were spotted on selective agar plates with or without 1 mM IPTG. After overnight incubation, the plates were compared to assess the sensitivity of the clones to IPTG.

Nucleic acids involved in proliferation of Pseudomonas aeruginosa were identified as follows. Randomly generated fragments of Pseudomonas aeruginosa genomic DNA were transcribed from a two-component inducible promoter system. Integrated on the chromosome was the T7 RNA polymerase gene regulated by lacUV5/lacO (Brunschwig, E. and Darzins, A. 1992. Gene 111:35-41). On an expression plasmid there was a T7 gene 10 promoter, which is transcribed by T7 RNA polymerase, fused with a lacO operator followed by a multiple cloning site. Transcription from this hybrid promoter is inducible by IPTG. Should the genomic DNA downstream of the promoter contain, in an antisense orientation, at least a portion of an mRNA encoding a gene product involved in proliferation, then induction of expression from the promoter will result in detectable inhibition of proliferation.

A shotgun library of Pseudomonas aeruginosa genomic fragments was cloned into the vectors pEP5, pEP5S, or other similarly constructed vectors which harbor the T7lacO inducible promoter. The vector was linearized at a unique SmaI site immediately downstream of the T7lacO promoter/operator. The linearized vector was treated with shrimp alkaline phosphatase to prevent reclosure of the linearized ends. Genomic DNA isolated from Pseudomonas aeruginosa strain PAO1 was partially digested with DNase I and “blunt-ended” by incubating with T4 DNA polymerase. Random genomic fragments between 200 and 800 base pairs in length were selected by gel purification. The size-selected genomic fragments were added to the linearized and dephosphorylated vector at a molar ratio of 2 to 1, and ligated to form a shotgun library.

The ligated products were transformed into electrocompetent E. coli strain XL1-Blue MRF (Stratagene) and plated on LB medium with carbenicillin at 100 μg/ml or Streptomycin 100 μg/ml. Resulting colonies numbering 5×10⁵ or greater were scraped and combined, and were then subjected to plasmid purification.

The purified library was then transformed into electrocompetent Pseudomonas aeruginosa strain PAO1. Resulting transformants were plated on LB agar with carbenicillin at 100 μg/ml or Streptomycin 40 μg/ml in order to generate 100 to 150 platings at 500 colonies per plating. The colonies were subjected to robotic picking and arrayed into wells of 384 well culture dishes. Each well contained 100 μl of LB+CB 100 or Streptomycin 40 liquid medium. Inoculated 384 well dishes were incubated 16 hours at room temperature, and each well was robotically gridded onto solid LB+CB100 or Streptomycin 40 medium with or without 1 mM IPTG. Gridded plates were incubated 16 hours at 37° C., and then manually scored for arrayed colonies that were growth-compromised in the presence of IPTG.

Arrayed colonies that were growth-sensitive on medium containing 1 mM IPTG, yet were able to grow on similar medium lacking IPTG, were subjected to further growth sensitivity analysis as follows: Colonies from the plate lacking IPTG were manually picked and inoculated into individual wells of a 96 well culture dish containing LB+CB100 or Streptomycin 40, and were incubated for 16 hours at 30° C. These cultures were robotically diluted 1/100 into fresh medium and allowed to incubate for 4 hours at 37° C., after which they were subjected to serial dilutions in a 384 well array and then gridded onto media with and without 1 mM IPTG. After growth for 16 hours at 37° C., the arrays of serially diluted spots that resulted were compared between the two media. Clones that grew similarly at all dilutions on both media were scored as a negative and were no longer considered. Clones that grew on IPTG medium but failed to grow at the same serial dilution on the non-IPTG plate were given a score based on the differential, i.e. should the clone grow at a serial dilution of 10⁴ or less on the IPTG plate and grow at a serial dilution of 10⁸ or less on the IPTG plate, then the corresponding clone received a score of “4” representing the log difference in growth observed.

Following the identification of those vectors that, upon induction, negatively impacted Pseudomonas aeruginosa growth or proliferation, the inserts or nucleic acid fragments contained in those vectors were isolated for subsequent characterization. Vectors of interest were subjected to nucleic acid sequence determination.

Nucleic acids involved in proliferation of E. faecalis were identified as follows. Randomly generated fragments of genomic DNA were expressed from the vectors pEPEF3 or pEPEF14, which contain the CP25 or P59 promoter, respectively, regulated by the xy1 operator/repressor. Should the genomic DNA downstream of the promoter contain, in an antisense orientation, at least a portion of a mRNA encoding a gene product involved in proliferation, then induction of expression from the promoter will result in detectable inhibition of proliferation.

A shotgun library of E. faecalis genomic fragments was cloned into the vector pEPEF3 or pEPEF14, which harbor xylose inducible promoters. The vector was linearized at a unique SmaI site immediately downstream of the promoter/operator. The linearized vector was treated with alkaline phosphatase to prevent reclosure of the linearized ends. Genomic DNA isolated from E. faecalis strain OG1RF was partially digested with DNase I and “blunt-ended” by incubating with T4 DNA polymerase. Random genomic fragments between 200 and 800 base pairs in length were selected by gel purification. The size-selected genomic fragments were added to the linearized and dephosphorylated vector at a molar ratio of 2 to 1, and ligated to form a shotgun library.

The ligated products were transformed into electrocompetent E. coli strain TOP10 cells (Invitrogen) and plated on LB medium with erythromycin (Erm) at 150 μg/ml. Resulting colonies numbering 5×10⁵ or greater were scraped and combined, and were then subjected to plasmid purification.

The purified library was then transformed into electrocompetent E. faecalis strain OGIRF. Resulting transformants were plated on Todd-Hewitt (TH) agar with erythromycin at 10 μg/ml in order to generate 100 to 150 platings at 500 colonies per plating. The colonies were subjected to robotic picking and arrayed into wells of 384 well culture dishes. Each well contained 100 μl of THB+Erm 10 μg/ml. Inoculated 384 well dishes were incubated 16 hours at room temperature, and each well was robotically gridded onto solid TH agar+Erm with or without 5% xylose. Gridded plates were incubated 16 hours at 37° C., and then manually scored for arrayed colonies that were growth-compromised in the presence of xylose.

Arrayed colonies that were growth-sensitive on medium containing 5% xylose, yet were able to grow on similar medium lacking xylose, were subjected to further growth sensitivity analysis. Colonies from the plate lacking xylose were manually picked and inoculated into individual wells of a 96 well culture dish containing THB+Erm 10, and were incubated for 16 hours at 30° C. These cultures were robotically diluted 1/100 into fresh medium and allowed to incubate for 4 hours at 37° C., after which they were subjected to serial dilution on plates containing 5% xylose or plates lacking xylose. After growth for 16 hours at 37° C., the arrays of serially diluted spots that resulted were compared between the two media. Colonies that grew similarly on both media were scored as a negative and corresponding colonies were no longer considered. Colonies on xylose medium that failed to grow to the same serial dilution compared to those on the non-xylose plate were given a score based on the differential. For example, colonies on xylose medium that only grow to a serial dilution of −4 while they were able to grow to −8 on the non-xylose plate, then the corresponding transformant colony received a score of “4” representing the log difference in growth observed.

Following the identification of those vectors that, upon induction, negatively impacted E. faecalis growth or proliferation, the inserts or nucleic acid fragments contained in those expression vectors were isolated for subsequent characterization. The inserts in the vectors of interest were subjected to nucleotide sequence determination.

It will be appreciated that other restriction enzymes and other endonucleases or methodologies may be used to generate random genomic fragments. In addition, random genomic fragments may be generated by mechanical shearing. Sonication and nebulization are two such techniques commonly used for mechanical shearing of DNA.

### Example 2

### Nucleotide Sequence Determination of Identified Clones Transribing Nucleic Acid Fragments with Detrimental Effects on Staphylococcus aureus, Salmonella typhimurium, Klebsiella pneumoniae, Pseudomonas aeruginosa or Enterococcus faecalis Proliferation

Plasmids from clones that received a dilution plating score of “2” or greater were isolated to obtain the genomic DNA insert responsible for growth inhibition as follows. Staphylococcus aureus were grown in standard laboratory media (LB or TB with 15 ug/ml Chloramphenicol to select for the plasmid). Growth was carried out at 37° C. overnight in culture tubes or 2 ml deep well microtiter plates.

Lysis of Staphylococcus aureus was performed as follows. Cultures (2-5 ml) were centrifuged and the cell pellets resuspended in 1.5 mg/ml solution of lysostaphin (20 μl/ml of original culture) followed by addition of 250 μl of resuspension buffer (Qiagen). Alternatively, cell pellets were resuspended directly in 250 μl of resuspension buffer (Qiagen) to which 5-20 μl of a 1 mg/ml lysostaphin solution were added.

DNA was isolated using Qiagen miniprep kits or Wizard (Qiagen) miniprep kits according to the instructions provided by the manufacturer.

The genomic DNA inserts were amplified from the purified plasmids by PCR as follows.

1 μl of Qiagen purified plasmid was put into a total reaction volume of 25 μl Qiagen Hot Start PCR mix. For Staphylococcus aureus, the following primers were used in the PCR reaction:

| | |
|---|---|
| pXylT5F: CAGCAGTCTGAGTTATAAAATAG | (SEQ ID NO: 1) |
| LexL TGTTTTATCAGACCGCTT | (SEQ ID NO: 2) |

Similar methods were conducted for Salmonella typhimurium and Klebsiella pneumoniae. For Salmonella typhimurium and Klebsiella pneumoniae the following primers were used:

| | |
|---|---|
| 5′-TGTTTTATCAGACCGCTT-3′ | (SEQ ID NO: 2) |
| and | |
| 5′-ACAATTTCACACAGCCTC-3′ | (SEQ ID NO: 4) |

PCR was carried out in a PE GenAmp with the following cycle times:

Step 1. 95° C. 15 min

Step 2. 94° C. 45sec

Step 3. 54° C. 45 sec

Step 4. 72° C. 1 minute

Step 5. Return to step 2, 29 times

Step 6. 72° C. 10 minutes

Step 7. 4° C. hold

The PCR products were cleaned using Qiagen Qiaquick PCR plates according to the manufacturer's instructions.

For Pseudomonas aeruginosa, plasmids from transformant colonies that received a dilution plating score of “2” or greater were isolated to obtain the genomic DNA insert responsible for growth inhibition as follows. Pseudomonas aeruginosa were grown in standard laboratory media (LB with carbenicillin at 100 μg/ml or Streptomycin 40 μg/ml to select for the plasmid). Growth was carried out at 30° C. overnight in 100 ul culture wells in microtiter plates. To amplify insert DNA 2 ul of culture were placed into 25 ul Qiagen Hot Start PCR mix. PCR reactions were in 96 well microtiter plates. For plasmid pEP5S the following primers were used in the PCR reaction:

| | |
|---|---|
| T7L1+: GTCGGCGATATAGGCGCCAGCAACCG | (SEQ ID NO: 5) |
| pStrA3: ATAATCGAGCATGAGTATCATACG | (SEQ ID NO: 6) |

PCR was carried out in a PE GenAmp with the following cycle times:

Step 1. 95° C. 15 min

Step 2. 94° C. 45 sec

Step 3. 54° C. 45 sec

Step 4. 72° C. 1 minute

Step 5. Return to step 2, 29 times

Step 6. 72° C. 10 minutes

Step 7. 4° C. hold

The PCR products were cleaned using Qiagen Qiaquick PCR plates according to the manufacturer's instructions.

The purified PCR products were then directly cycle sequenced with Qiagen Hot Start PCR mix. The following primers were used in the sequencing reaction:

| | |
|---|---|
| T7/L2: ATGCGTCCGGCGTAGAGGAT | (SEQ ID NO: 7) |

PCR was carried out in a PE GenAmp with the following cycle times:

Step 1. 94° C. 15 min

Step 2. 96° C. 10 sec

Step 3. 50° C. 5 sec

Step 4. 60° C. 4 min

Step 5. Return to step 2, 24 times

Step 6. 4° C. hold

The PCR products were cleaned using Qiagen Qiaquick PCR plates according to the manufacturer's instructions.

For E. faecalis, plasmids from transformant colonies that received a dilution plating score of “2” or greater were isolated to obtain the genomic DNA insert responsible for growth inhibition as follows. E. faecalis were grown in THB 10 μg/ml Erm at 30° C. overnight in 100 ul culture wells in microtiter plates. To amplify insert DNA 2 ul of culture were placed into 25 μl Qiagen Hot Start PCR mix. PCR reactions were in 96 well microtiter plates. The following primers were used in the PCR reaction:

| | |
|---|---|
| pXylT5: CAGCAGTCTGAGTTATAAAATAG | (SEQ ID NO: 1) |
| and the | |

PCR was carried out in a PE GenAmp with the following cycle times:

Step 1. 95° C. 15 min

Step 2. 94° C. 45 sec

Step 3. 54° C. 45 sec

Step 4. 72° C. 1 minute

Step 5. Return to step 2, 29 times

Step 6. 72° C. 10 minutes

Step 7. 4° C. hold

The PCR products were cleaned using Qiagen Qiaquick PCR plates according to the manufacturer's instructions.

The purified PCR products were then directly cycle sequenced with Qiagen Hot Start PCR mix. The following primers were used in the PCR reaction:

| | |
|---|---|
| pXylT5: CAGCAGTCTGAGTTATAAAATAG | (SEQ ID NO: 1) |

PCR was carried out in a PE GenAmp with the following cycle times:

Step 1. 94° C. 15 min

Step 2. 96° C. 10 sec

Step 3. 50° C. 5 sec

Step 4. 60° C. 4 min

Step 5. Return to step 2, 24 times

Step 6. 4° C. hold

The PCR products were cleaned using Qiagen Qiaquick PCR plates according to the manufacturer's instructions.

The amplified genomic DNA inserts from each of the above procedures were subjected to automated sequencing. Sequence identification numbers (SEQ ID NOs) and clone names for the identified inserts are listed in Table IA and discussed below.

### Example 3

### Comparison of Isolated Nucleic Acids to Known Sequences

The nucleotide sequences of the subcloned fragments from Staphylococcus aureus, Salmonella typhimurium, Klebsiella pneumoniae, Pseudomonas aeruginosa or Enterococcus faecalis obtained from the expression vectors discussed above were compared to known sequences from Staphylococcus aureus, Salmonella typhimurium, Klebsiella pneumoniae, Pseudomonas aeruginosa or Enterococcus faecalis and other microorganisms as follows. First, to confirm that each clone originated from one location on the chromosome and was not chimeric, the nucleotide sequences of the selected clones were compared against the Staphylococcus aureus, Salmonella typhimurium, Klebsiella pneumoniae, Pseudomonas aeruginosa or Enterococcus faecalis genomic sequences to align the clone to the correct position on the chromosome. The NCBI BLASTN v 2.0.9 program was used for this comparison, and the incomplete Staphylococcus aureus genomic sequences licensed from TIGR, as well as the NCBI nonredundant GenBank database were used as the source of genomic data. Salmonella typhimurium sequences were compared to sequences available from the Genome Sequencing Center (http://genome.wustl.edu/gsc/salmonella.shtml), and the Sanger Centre (http://www.sanger.ac.uk/projects/S_typhi). Pseudomonas aeruginosa sequences were compared to a proprietary database and the NCBI GenBank database. The E. faecalis sequences were compared to a proprietary database.

The BLASTN analysis was performed using the default parameters except that the filtering was turned off. No further analysis was performed on inserts which resulted from the ligation of multiple fragments.

In general, antisense molecules and their complementary genes are identified as follows. First, all possible full length open reading frames (ORFs) are extracted from available genomic databases. Such databases include the GenBank nonredundant (nr) database, the unfinished genome database available from TIGR and the PathoSeq database developed by Incyte Genomics. The latter database comprises over 40 annotated bacterial genomes including complete ORF analysis. If databases are incomplete with regard to the bacterial genome of interest, it is not necessary to extract all ORFs in the genome but only to extract the ORFs within the portions of the available genomic sequences which are complementary to the clones of interest. Computer algorithms for identifying ORFs, such as GeneMark, are available and well known to those in the art. Comparison of the clone DNA to the complementary ORF(s) allows determination of whether the clone is a sense or antisense clone. Furthermore, each ORF extracted from the database can be compared to sequences in well annotated databases including the GenBank (nr) protein database, SWISSPROT and the like. A description of the gene or of a closely related gene in a closely related microorganism is often available in these databases. Similar methods are used to identify antisense clones corresponding to genes encoding non-translated RNAs.

In order to generate the gene identification data compiled in Table IB, each of the cloned nucleic acid sequences discussed above corresponding to SEQ ID NO.s 8-3795 was used to identify the corresponding Staphylococcus aureus, Salmonella typhimurium, Klebsiella pneumoniae, Pseudomonas aeruginosa or Enterococcus faecalis ORFs in the PathoSeq v.4.1 (March 2000 release) database of microbial genomic sequences. For this purpose, the NCBI BLASTN 2.0.9 computer algorithm was used. The default parameters were used except that filtering was turned off. The default parameters for the BLASTN and BLASTX analyses were:

Expectation value (e)=10

Alignment view options: pairwise

Filter query sequence (DUST with BLASTN, SEG with others)=T

Cost to open a gap (zero invokes behavior)=0

Cost to extend a gap (zero invokes behavior)=0

X dropoff value for gapped alignment (in bits) (zero invokes behavior)=0

Show GI's in deflines=F

Penalty for a nucleotide mismatch (BLASTN only)=!3

Reward for a nucleotide match (BLASTN only)=1

Number of one-line descriptions (V)=500

Number of alignments to show (B)=250

Threshold for extending hits=default

Perform gapped alignment (not available with BLASTX)=T

Query Genetic code to use=1

DB Genetic code (for TBLAST[nx] only=1

Number of processors to use=1

SeqAlign file

Believe the query defline=F

Matrix=BLOSUM62

Word Size=default

Effective length of the database (use zero for the real size)=0

Number of best hits from a region to keep=100

Length of region used to judge hits=20

Effective length of the search space (use zero for the real size)=0

Query strands to search against database (for BLAST[nx] and TBLASTX), 3 is both, 1 is top, 2 is bottom=3

Produce HTML output=F

Alternatively, ORFs were identified and refined by conducting a survey of the public and private data sources. Full-length gene protein and nucleotide sequences for these organisms were assembled from various sources. For Pseudomonas aeruginosa, gene sequences were adopted from the Pseudomonas genome sequencing project (downloaded from http://www.pseudomonas.com). For Klebsiella pneumoniae, Staphylococcus aureus, Streptococcus pneumoniae and Salmonella typhi, genomic sequences from PathoSeq v 4.1 (Mar 2000 release) was reanalyzed for ORFs using the gene finding software GeneMark v 2.4a, which was purchased from GenePro Inc. 451 Bishop St., N. W., Suite B, Atlanta, Ga., 30318, USA.

Antisense clones were identified as those clones for which transcription from the inducible promoter would result in the expression of an RNA antisense to a complementary ORF, intergenic or intragenic sequence. Those clones containing single inserts and that caused growth sensitivity upon induction are listed in Table IA. ORFs complementary to the antisense nucleic acids, and their encoded polypeptides, are listed in Table IB.

The gene descriptions in the PathoSeq database derive from annotations available in the public sequence databases described above. Where a clone was found to share significant sequence identity to two or more adjacent ORFs, it was listed once for each ORF and the PathoSeq information for each ORF was compiled in Table IB.

Table IA lists the SEQ ID NOs. and clone names of the inserts which inhibited proliferation and the organism in which the clone was identified. This information was used to identify the ORFs (SEQ ID NOs.: 3796-3800, 3806-4860, 5916-10012) whose gene products (SEQ ID NOs. 3801-3805, 4861-5915, 10013-14110) were inhibited by the nucleic acids comprising the nucleotide sequences of SEQ ID NOs. 8-3795. Table IB lists the clone name, the SEQ ID NO. of the antisense clone (in the column labelled Clone SEQ ID), the PathoSeq Locus containing the clone, the SEQ ID of the ORF identified in PathoSeq (in the column labelled Gene Seq ID (protein), the refined full length gene (column labelled genemarked gene), and the SEQ ID NO of the protein encoded by the refined full length gene (column labelled full length ORF protein SEQ ID).

Table IC provides a cross reference between PathoSeq Gene Locus listed in Table IB, the SEQ ID NOs. of the PathoSeq proteins and the SEQ ID NOs. of the nucleic acids which encode them.

It will be appreciated that ORFs may also be identified using databases other than PathoSeq. For example, the ORFs may be identified using the methods described in U.S. Provisional Patent Application Ser. No. 60/191,078, filed Mar. 21, 2000, the disclosure of which is incorporated herein by reference in its entirety.

### Example 4

### Identification of Genes and their Corresponding Operons Affected by Antisense Inhibition

Once the genes involved in Staphylococcus aureus, Salmonella typhimurium, Klebsiella pneumoniae, Pseudomonas aeruginosa or Enterococcus faecalis proliferation are identified as described above, the operons in which these genes lie may be identified by comparison with known microbial genomes. Since bacterial genes are transcribed in a polycistronic manner, the antisense inhibition of a single gene in an operon might affect the expression of all the other genes on the operon or the genes downstream from the single gene identified. Accordingly, each of the genes contained within an operon may be analyzed for their effect on proliferation.

Operons are predicted by looking for all adjacent genes in a genomic region that lie in the same orientation with no large noncoding gaps in between. First, full-length ORFs complementary to the antisense molecules are identified as described above. Adjacent ORFs are then identified and their relative orientation determined either by directly analyzing the genomic sequences surrounding the ORFs complementary to the antisense clones or by extracting adjacent ORFs from the collection obtained through whole genome ORF analysis described above followed by ORF alignment. Operons predicted in this way may be confirmed by comparison to the arrangement of the homologous nucleic acids in the Bacillus subtilis complete genome sequence, as reported by the genome database compiled at Institut Pasteur Subtilist Release RI 5.1 (Jun. 24, 1999) which can be found at htt ://bioweb.pasteur.fr/GenoList/SubtiList/. The Bacillus subtilis genome is the only fully sequenced and annotated genome from a Gram-positive microorganism, and appears to have a high level of similarity to Staphylococcus aureus both at the level of conservation of gene sequence and genomic organization including operon structure. Operons for Salmonella typhimurium and Klebsiella pneumoniae may be identified by comparison with E. coli, Haemophilus, or Pseudomonas sequences. The Pseudomonas aeruginosa web site (http://www.pseudomonas.com) can also be used to help predict operon organization in this bacterium.

Extensive DNA sequences of Salmonella typhimurium are available through the Salmonella Genome Center (Washington University, St. Louis, Mo.) the Sanger Centre (United Kingdom) and the PathoSeq database (Incyte ). Annotation of some of the DNA sequences in some of the aforementioned databases is lacking, but comparisons may be made to E. coli using tools such as BLASTX.

Public or proprietary databases may be used to analyzed E. faecalis sequences as well as sequences from the organisms listed above.

The results of such an analysis as applied to clone number S1M10000001A05 from Staphylococcus aureus are listed in Table II. Table II lists the SEQ ID NOs. of the Staphylococcus aureus genes involved in proliferation, the SEQ ID NOs. of the proteins encoded by these genes, and the clone name containing the nucleic acid which inhibits Staphylococcus aureus proliferation. In addition, Table II lists those other genes located on the operon included in the Staphylococcus aureus genomic sequence determined as described above. For each of the genes described in Table II, the microoganism containing the most closely related homolog, identified in one of the public databases, is also indicated in Table II.

TABLE II

| | | | | | Organism |
|---|---|---|---|---|---|
| | | | | | used for |
| DNA | Protein | Molecule | | | identification |
| Seq ID | Seq ID | number | Clone name | Gene | of gene |
| 3796 | 3801 | SaXA001 | S1M10000001A05 | ytmI | B. subtilis |
| 3797 | 3802 | | | nirR | S. carnosus |
| 3798 | 3803 | | | nirB | S. carnosus |
| 3799 | 3804 | | | nirD | S. carnosus |
| 3800 | 3805 | | | sirB | S. carnosus |

The preceding analyses may be conducted for each of the sequences which are listed in Table IA which inhibit proliferation and the ORFs listed in Table IB and Table IC. Once the full length ORFs and/or the operons containing them have been identified using the methods described above, they can be obtained from a genomic library by performing a PCR amplification using primers at each end of the desired sequence. Those skilled in the art will appreciate that a comparison of the ORFs to homologous sequences in other cells or microorganisms will facilitate confirmation of the start and stop codons at the ends of the ORFs.

In some embodiments, the primers may contain restriction sites which facilitate the insertion of the gene or operon into a desired vector. For example, the gene may be inserted into an expression vector and used to produce the proliferation-required protein as described below. Other methods for obtaining the full length ORFs and/or operons are familiar to those skilled in the art. For exmaple, natural restriction sites may be employed to insert the full length ORFs and/or operons into a desired vector.

### Example 5

### Identification of Individual Genes within an Operon Required for Proliferation

The following example illustrates a method for determining if a targeted gene within an operon is required for cell proliferation by replacing the targeted allele in the chromosome with an in-frame deletion of the coding region of the targeted gene.

Deletion inactivation of a chromosomal copy of a gene in Staphylococcus aureus, Salmonella typhimurium, Klebsiella pneumoniae, Pseudomonas aeruginosa, Enterococcus faecalis, Escherichia coli, Enterococcus faecalis, Haemophilus influenzae, Helicobacter pylori, or Salmonella typhi can be accomplished by integrative gene replacement. The principles of this method were described in Xia, M., et al. 1999 Plasmid 42:144-149 and Hamilton, C. M., et al 1989. J Bacteriol. 171: 4617-4622, the disclosures of which are incorporated herein by reference in their entireties. A similar gene disruption method is available for Pseudomonas aeruginosa, except the counter selectable marker is sacB (Schweizer, H. P., Klassen, T. and Hoang, T. (1996) Mol. Biol. of Pseudomonas. ASM press, 229-237, the disclosure of which is incorporated herein by reference in its entirety). In this approach, a mutant allele of the targeted gene is constructed by way of an in-frame deletion and introduced into the chromosome using a suicide vector. This results in a tandem duplication comprising a deleted (null) allele and a wild type allele of the target gene. Cells in which the vector sequences have been deleted are isolated using a counter-selection technique. Removal of the vector sequence from the chromosomal insertion results in either restoration of the wild-type target sequence or replacement of the wild type sequence with the deletion (null) allele. E. faecalis genes can be disrupted using a suicide vector that contains an internal fragment to a gene of interest. With the appropriate selection this plasmid will homologously recombine into the chromosome (Nallapareddy, S. R., X. Qin, G. M. Weinstock, M. Hook, B. E. Murray. 2000. Infect. Immun. 68:5218-5224, the disclosure of which is incorporated herein by reference).

The resultant population of Staphylococcus aureus, Salmonella typhimurium, Klebsiella pneumoniae, Pseudomonas aeruginosa, Enterococcus faecalis, Escherichia coli, Enterococcus faecalis, Haemophilus influenzae, Helicobacter pylori, or Salmonella typhi colonies can then be evaluated to determine whether the target sequence is required for proliferation by PCR amplification of the affected target sequence. If the targeted gene is not required for proliferation, then PCR analysis will show that roughly equal numbers of colonies have retained either the wild-type or the mutant allele. If the targeted gene is required for proliferation, then only wild-type alleles will be recovered in the PCR analysis.

The method of cross-over PCR is used to generate the mutant allele by amplification of nucleotide sequences flanking but not including the coding region of the gene of interest, using specifically designed primers such that overlap between the resulting two PCR amplification products allows them to hybridize. Further PCR amplification of this hybridization product using primers representing the extreme 5′ and 3′ ends can produce an amplification product containing an in-frame deletion of the coding region but retaining substantial flanking sequences.

For Staphylococcus aureus, this amplification product is subcloned into the suicide vector pSA3182 (Xia, M., et al. 1999 Plasmid 42:144-149, the disclosure of which is incorporated herein by reference in its entirety) which is host-dependent for autonomous replication. This vector includes a tetC tetracycline-resistance marker and the origin of replication of the well-known Staphylococcus aureus plasmid pT181 (Mojumdar, M and Kahn, S. A., Characterisation of the Tetracycline Resistance Gene of Plasmid pT181, J. Bacteriol. 170: 5522 (1988), the disclosure of which is incorporated herein by reference in its entirety). The vector lacks the repC gene which is required for autonomous replication of the vector at the pT181 origin. This vector can be propagated in a Staphylococcus aureus host strain such as SA3528, which expresses repC in trans. Once the amplified truncated target gene sequence is cloned and propagated in the pSA3182 vector, it can then be introduced into a repC minus strain such as RN4220 (Kreiswirth, B. N. et al., The Toxic Shock Syndrome Exotoxin Structural Gene is Not Detectably Transmitted by a Prophage, Nature 305:709-712 (1983), the disclosure of which is incorporated herein by reference in its entirety) by electroporation with selection for tetracycline resistance. In this strain, the vector must integrate by homologous recombination at the targeted gene in the chromosome to impart drug resistance. This results in a inserted truncated copy of the allele, followed by pSA3182 vector sequence, and finally an intact and functional allele of the targeted gene.

Once a tetracycline resistant Staphylococcus aureus strain is isolated using the above technique and shown to include truncated and wild-type alleles of the targeted gene as described above, a second plasmid, pSA7592 (Xia, M., et al. 1999 Plasmid 42:144-149, the disclosure of which is incorporated herein by reference in its entirety) is introduced into the strain by electroporation. This gene includes an erythromycin resistance gene and a repC gene that is expressed at high levels. Expression of repC in these transformants is toxic due to interference of normal chromosomal replication at the integrated pT181 origin of replication. This selects for strains that have removed the vector sequence by homologous recombination, resulting in either of two outcomes: The selected cells either possess a wild-type allele of the targeted gene or a gene in which the wild-type allele has been replaced by the engineered in-frame deletion of the truncated allele.

PCR amplification can be used to determine the genetic outcome of the above process in the resulting erythromycin resistant, tet sensitive transformant colonies. If the targeted gene is not required for cellular replication, then PCR evidence for both wild-type and mutant alleles will be found among the population of resultant transformants. However, if the targeted gene is required for cellular proliferation, then only the wild-type form of the gene will be evident among the resulting transformants.

Similarly, for Salmonella typhimurium, Klebsiella pneumoniae, Pseudomonas aeruginosa or Enterococcus faecalis, Escherichia coli Enterococcus faecalis, Haemophilus influenzae, Helicobacter pylori, or Salmonella typhi the PCR products containing the mutant allele of the target sequence may be introduced into an appropriate knockout vector and cells in which the wild type target has been disrupted are selected using the appropriate methodology.

The above methods have the advantage that insertion of an in-frame deletion mutation is far less likely to cause downstream polar effects on genes in the same operon as the targeted gene. However, it will be appreciated that other methods for disrupting Staphylococcus aureus, Salmonella typhimurium, Klebsiella pneumoniae, Pseudomonas aeruginosa, Enterococcus faecalis, Escherichia coli, Enterococcus faecalis, Haemophilus influenzae, Helicobacter pylori, or Salmonella typhi genes which are familiar to those skilled in the art may also be used.

Each gene in the operon may be disrupted using the methodology above to determine whether it is required for proliferation.

### Example 6

### Expression of the Proteins Encoded by Genes Identified as Required for Staphylococcus aureus, Salmonella typhimurium, Klebsiella pneumoniae, Pseudomonas aeruginosa, Enterococcus faecalis, Escherichia coli, Enterococcus faecalis, Haemophilus influenzae, Helicobacter pylori, or Salmonella typhi Proliferation

The following is provided as one exemplary method to express the proliferation-required proteins idenfied as described above. The proliferation-required proteins may be expressed using any of the bacterial, insect, yeast, or mammalian expression systems known in the art. In some embodiments, the proliferation-required proteins encoded by the identified nucleotide sequences described above (including the proteins of SEQ ID NOs.: 3801-3805,4861-5915, 10013-14110 encoded by the nucleic acids of SEQ ID NOs.: 3796-3800, 3806-4860, 5916-10012 are expressed using expression systems designed either for E. coli or for Staphylococcus aureus, Salmonella typhimurium, Klebsiella pneumoniae, Pseudomonas aeruginosa, Enterococcus faecalis, Enterococcus faecalis, Haemophilus influenzae, Helicobacter pylori, or Salmonella typhi. First, the initiation and termination codons for the gene are identified. If desired, methods for improving translation or expression of the protein are well known in the art. For example, if the nucleic acid encoding the polypeptide to be expressed lacks a methionine codon to serve as the initiation site, a strong Shine-Delgarno sequence, or a stop codon, these nucleotide sequences can be added. Similarly, if the identified nucleic acid lacks a transcription termination signal, this nucleotide sequence can be added to the construct by, for example, splicing out such a sequence from an appropriate donor sequence. In addition, the coding sequence may be operably linked to a strong constitutive promoter or an inducible promoter if desired. The identified nucleic acid or portion thereof encoding the polypeptide to be expressed is obtained by, for example, PCR from the bacterial expression vector or genome using oligonucleotide primers complementary to the identified nucleic acid or portion thereof and containing restriction endonuclease sequences appropriate for inserting the coding sequences into the vector such that the coding sequences can be expressed from the vector's promoter. Alternatively, other conventional cloning techniques may be used to place the coding sequence under the control of the promoter. In some embodiments, a termination signal may be located downstream of the coding sequence such that transcription of the coding sequence ends at an appropriate position.

Several expression vector systems for protein expression in E. coli are well known and available to those knowledgeable in the art. The coding sequence may be inserted into any of these vectors and placed under the control of the promoter. The expression vector may then be transformed into DH5α or some other E. coli strain suitable for the over expression of proteins.

Alternatively, an expression vector encoding a protein required for proliferation of Staphylococcus aureus, Salmonella typhimurium, Klebsiella pneumoniae, Pseudomonas aeruginosa, Enterococcus faecalis, Escherichia coli, Enterococcus faecalis, Haemophilus influenzae, Helicobacter pylori, or Salmonella typhi may be introduced into Staphylococcus aureus, Salmonella typhimurium, Klebsiella pneumoniae, Pseudomonas aeruginosa, Enterococcus faecalis, Escherichia coli, Enterococcus faecalis, Haemophilus influenzae, Helicobacter pylori, or Salmonella typhi. Protocols for introducing nucleic acids into these organisms are well known in the art. For example, the protocols described in J. C. Lee “Electroporation of Staphylococci” from Methods in Molecular Biology vol 47: Electroporation Protocols for Microorganisms Edited by: J. A. Nickoloff Humana Press Inc., Totowa, N.J. pp209-216, the disclosure of which is incorporated herein by reference in its entirety, may be used to introduce nucleic acids into Staphylococcus aureus. Nucleic acids may also be introduced into Salmonella typhimurium, Klebsiella pneumoniae, Pseudomonas aeruginosa or Enterococcus faecalis using methods familiar to those skilled in the art. Positive transformants are selected after growing the transformed cells on plates containing an antibiotic to which the vector confers resistance. In one embodiment, Staphylococcus aureus is transformed with an expression vector in which the coding sequence is operably linked to the T5 promoter containing a xylose operator such that expression of the encoded protein is inducible with xylose.

In one embodiment, the protein is expressed and maintained in the cytoplasm as the native sequence. In an alternate embodiment, the expressed protein can be modified to include a protein tag that allows for differential cellular targeting, such as to the periplasmic space of Gram-negative or Gram-positive expression hosts or to the exterior of the cell (i.e., into the culture medium). In some embodiments, the osmotic shock cell lysis method described in Chapter 16 of Current Protocols in Molecular Biology, Vol. 2, (Ausubel, et al., Eds.) John Wiley & Sons, Inc. (1997) may be used to liberate the polypeptide from the cell. In still another embodiment, such a protein tag could also facilitate purification of the protein from either fractionated cells or from the culture medium by affinity chromatography. Each of these procedures can be used to express a proliferation-required protein.

Expressed proteins, whether in the culture medium or liberated from the periplasmic space or the cytoplasm, are then purified or enriched from the supernatant using conventional techniques such as ammonium sulfate precipitation, standard chromatography, immunoprecipitation, immunochromatography, size exclusion chromatography, ion exchange chromatography, and HPLC. Alternatively, the polypeptide may be secreted from the host cell in a sufficiently enriched or pure state in the supernatant or growth media of the host cell to permit it to be used for its intended purpose without further enrichment. The purity of the protein product obtained can be assessed using techniques such as SDS PAGE, which is a protein resolving technique well known to those skilled in the art. Coomassie, silver staining or staining with an antibody are typical methods used to visualize the protein of interest.

Antibodies capable of specifically recognizing the protein of interest can be generated using synthetic peptides using methods well known in the art. See, Antibodies: A Laboratory Manual, (Harlow and Lane, Eds.) Cold Spring Harbor Laboratory (1988). For example, 15-mer peptides having an amino acid sequence encoded by the appropriate identified gene sequence of interest or portion thereof can be chemically synthesized. The synthetic peptides are injected into mice to generate antibodies to the polypeptide encoded by the identified nucleic acid sequence of interest or portion thereof. Alternatively, samples of the protein expressed from the expression vectors discussed above can be purified and subjected to amino acid sequencing analysis to confirm the identity of the recombinantly expressed protein and subsequently used to raise antibodies. An Example describing in detail the generation of monoclonal and polyclonal antibodies appears in Example 7.

The protein encoded by the identified nucleic acid of interest or portion thereof can be purified using standard immunochromatography techniques. In such procedures, a solution containing the secreted protein, such as the culture medium or a cell extract, is applied to a column having antibodies against the secreted protein attached to the chromatography matrix. The secreted protein is allowed to bind the immunochromatography column. Thereafter, the column is washed to remove non-specifically bound proteins. The specifically-bound secreted protein is then released from the column and recovered using standard techniques. These procedures are well known in the art.

In an alternative protein purification scheme, the identified nucleic acid of interest or portion thereof can be incorporated into expression vectors designed for use in purification schemes employing chimeric polypeptides. In such strategies the coding sequence of the identified nucleic acid of interest or portion thereof is inserted in-frame with the gene encoding the other half of the chimera. The other half of the chimera can be maltose binding protein (MBP) or a nickel binding polypeptide encoding sequence. A chromatography matrix having maltose or nickel attached thereto is then used to purify the chimeric protein. Protease cleavage sites can be engineered between the MBP gene or the nickel binding polypeptide and the identified expected gene of interest, or portion thereof. Thus, the two polypeptides of the chimera can be separated from one another by protease digestion.

One useful expression vector for generating maltose binding protein fuision proteins is pMAL (New England Biolabs), which encodes the malE gene. In the pMa1 protein fusion system, the cloned gene is inserted into a pMa1 vector downstream from the malE gene. This results in the expression of an MBP-fusion protein. The fusion protein is purified by affinity chromatography. These techniques as described are well known to those skilled in the art of molecular biology.

### Example 7

### Production of an Antibody to an Isolated Staphylococcus aureus, Salmonella typhimurium, Klebsiella pneumoniae, Pseudomonas aeruginosa Enterococcus faecalis, Escherichia coli, Enterococcus faecalis, Haemophilus influenzae, Helicobacter pylori, or Salmonella typhi Protein

Substantially pure protein or polypeptide (including one of the polypeptides of SEQ ID NOs.: 3801-3805, 4861-5915, 10013-14110) is isolated from the transformed cells as described in Example 6. The concentration of protein in the final preparation is adjusted, for example, by concentration on a 10,000 molecular weight cut off AMICON filter device (Millipore, Bedford, Mass.), to the level of a few micrograms/ml. Monoclonal or polyclonal antibody to the protein can then be prepared as follows:

Monoclonal Antibody Production by Hybridoma Fusion

Monoclonal antibody to epitopes of any of the peptides identified and isolated as described can be prepared from murine hybridomas according to the classical method of Kohler, G. and Milstein, C., Nature 256:495 (1975) or any of the well-known derivative methods thereof. Briefly, a mouse is repetitively inoculated with a few micrograms of the selected protein or peptides derived therefrom over a period of a few weeks. The mouse is then sacrificed, and the antibody-producing cells of the spleen isolated. The spleen cells are fused by means of polyethylene glycol with mouse myeloma cells, and the excess unfused cells are destroyed by growth of the system on selective medium comprising aminopterin (HAT medium). The successfully-fused cells are diluted and aliquots of the dilution placed in wells of a microtiter plate where growth of the culture is continued. Antibody-producing clones are identified by detection of antibody in the supernatant fluid of the wells by immunoassay procedures, such as ELISA, as described by Engvall, E., “Enzyme immunoassay ELISA and EMIT,” Meth. Enzymol. 70:419 (1980), and derivative methods thereof. Selected positive clones can be expanded and their monoclonal antibody product harvested for use. Detailed procedures for monoclonal antibody production are described in Davis, L. et al. Basic Methods in Molecular Biology Elsevier, New York. Section 21-2.

Polyclonal Antibody Production by Immunization

Polyclonal antiserum containing antibodies to heterogeneous epitopes of a single protein or a peptide can be prepared by immunizing suitable animals with the expressed protein or peptides derived therefrom described above, which can be unmodified or modified to enhance immunogenicity. Effective polyclonal antibody production is affected by many factors related both to the antigen and the host species. For example, small molecules tend to be less immunogenic than larger molecules and can require the use of carriers and adjuvant. Also, host animals vary in response to site of inoculations and dose, with both inadequate or excessive doses of antigen resulting in low titer antisera. Small doses (ng level) of antigen administered at multiple intradermal sites appears to be most reliable. An effective immunization protocol for rabbits can be found in Vaitukaitis, J. et al. J. Clin. Endocrinol. Metab. 33:988-991 (1971).

Booster injections can be given at regular intervals, and antiserum harvested when antibody titer thereof, as determined semi-quantitatively, for example, by double immunodiffusion in agar against known concentrations of the antigen, begins to fall. See, for example, Ouchterlony, O. et al., Chap. 19 in: Handbook of Experimental Immunology D. Wier (ed) Blackwell (1973). Plateau concentration of antibody is usually in the range of 0.1 to 0.2 mg/ml of serum (about 12 EM). Affinity of the antisera for the antigen is determined by preparing competitive binding curves, as described, for example, by Fisher, D., Chap. 42 in: Manual of Clinical Immunology, 2d Ed. (Rose and Friedman, Eds.) Amer. Soc. For Microbiol., Washington, D.C. (1980).

Antibody preparations prepared according to either protocol are useful in quantitative immunoassays which determine concentrations of antigen-bearing substances in biological samples; they are also used semi-quantitatively or qualitatively to identify the presence of antigen in a biological sample. The antibodies can also be used in therapeutic compositions for killing bacterial cells expressing the protein.

### Example 8

### Screening Chemical Libraries

A. Protein-based Assays

Having isolated and expressed bacterial proteins shown to be required for bacterial proliferation, the present invention further contemplates the use of these expressed target proteins in assays to screen libraries of compounds for potential drug candidates. The generation of chemical libraries is well known in the art. For example, combinatorial chemistry can be used to generate a library of compounds to be screened in the assays described herein. A combinatorial chemical library is a collection of diverse chemical compounds generated by either chemical synthesis or biological synthesis by combining a number of chemical “building block” reagents. For example, a linear combinatorial chemical library such as a polypeptide library is formed by combining amino acids in every possible combination to yield peptides of a given length. Millions of chemical compounds theoretically can be synthesized through such combinatorial mixings of chemical building blocks. For example, one commentator observed that the systematic, combinatorial mixing of 100 interchangeable chemical building blocks results in the theoretical synthesis of 100 million tetrameric compounds or 10 billion pentameric compounds. (Gallop et al., “Applications of Combinatorial Technologies to Drug Discovery, Background and Peptide Combinatorial Libraries,” Journal of Medicinal Chemistry, Vol. 37, No. 9, 1233-1250 (1994). Other chemical libraries known to those in the art may also be used, including natural product libraries.

Once generated, combinatorial libraries can be screened for compounds that possess desirable biological properties. For example, compounds which may be useful as drugs or to develop drugs would likely have the ability to bind to the target protein identified, expressed and purified as discussed above. Further, if the identified target protein is an enzyme, candidate compounds would likely interfere with the enzymatic properties of the target protein. For example, the enzymatic function of a target protein may be to serve as a protease, nuclease, phosphatase, dehydrogenase, transporter protein, transcriptional enzyme, and any other type of enzyme known or unknown. Thus, the present invention contemplates using the protein products described above to screen combinatorial chemical libraries.

In one example, the target protein is a serine protease and the substrate of the enzyme is known. The present example is directed towards the analysis of libraries of compounds to identify compounds that function as inhibitors of the target enzyme. First, a library of small molecules is generated using methods of combinatorial library formation well known in the art. U.S. Pat. Nos. 5,463,564 and 5,574,656, to Agrafiotis, et al., entitled “System and Method of Automatically Generating Chemical Compounds with Desired Properties,” the disclosures of which are incorporated herein by reference in their entireties, are two such teachings. Then the library compounds are screened to identify those compounds that possess desired structural and functional properties. U.S. Pat. No. 5,684,711, the disclosure of which is incorporated herein by reference in its entirety, also discusses a method for screening libraries.

To illustrate the screening process, the target polypeptide and chemical compounds of the library are combined with one another and permitted to interact with one another. A labeled substrate is added to the incubation. The label on the substrate is such that a detectable signal is emitted from the products of the substrate molecules that result from the activity of the target polypeptide. The emission of this signal permits one to measure the effect of the combinatorial library compounds on the enzymatic activity of target enzymes by comparing it to the signal emitted in the absence of combinatorial library compounds. The characteristics of each library compound are encoded so that compounds demonstrating activity against the enzyme can be analyzed and features common to the various compounds identified can be isolated and combined into future iterations of libraries.

Once a library of compounds is screened, subsequent libraries are generated using those chemical building blocks that possess the features shown in the first round of screen to have activity against the target enzyme. Using this method, subsequent iterations of candidate compounds will possess more and more of those structural and functional features required to inhibit the function of the target enzyme, until a group of enzyme inhibitors with high specificity for the enzyme can be found. These compounds can then be further tested for their safety and efficacy as antibiotics for use in manmmals.

It will be readily appreciated that this particular screening methodology is exemplary only. Other methods are well known to those skilled in the art. For example, a wide variety of screening techniques are known for a large number of naturally-occurring targets when the biochemical function of the target protein is known. For example, some techniques involve the generation and use of small peptides to probe and analyze target proteins both biochemically and genetically in order to identify and develop drug leads. Such techniques include the methods described in PCT publications No. WO9935494, WO9819162, WO9954728, the disclosures of which are incorporated herein by reference in their entireties. Other techniques utilize natural product libraries or libraries of larger molecules such as proteins.

It will be appreciated that the above protein-based assays may be performed with any of the proliferation-required polypeptides from Staphylococcus aureus, Salmonella typhimurium, Klebsiella pneumoniae, Pseudomonas aeruginosa, Enterococcus faecalis, Escherichia coli, Enterococcus faecalis, Haemophilus influenzae, Helicobacter pylori, or Salmonella typhi (including the polypeptides of SEQ ID NOs.: 3801-3805, 4861-5915, 10013-14110) or portions thereof. In addition, the above protein-based assays may be performed with homologous polypeptides or portions thereof.

B. Cell-based Assays

Current cell-based assays used to identify or to characterize compounds for drug discovery and development frequently depend on detecting the ability of a test compound to modulate the activity of a target molecule located within a cell or located on the surface of a cell. An advantage of cell-based assays is that they allow the effect of a compound on a target molecule's activity to be detected within the physiologically relevant environment of the cell as opposed to an in vitro environment. Most often such target molecules are proteins such as enzymes, receptors and the like. However, target molecules may also include other molecules such as DNAs, lipids, carbohydrates and RNAs including messenger RNAs, ribosomal RNAs, tRNAs, regulatory RNAs and the like. A number of highly sensitive cell-based assay methods are available to those of skill in the art to detect binding and interaction of test compounds with specific target molecules. However, these methods are generally not highly effective when the test compound binds to or otherwise interacts with its target molecule with moderate or low affinity. In addition, the target molecule may not be readily accessible to a test compound in solution, such as when the target molecule is located inside the cell or within a cellular compartment. Thus, current cell-based assay methods are limited in that they are not effective in identifying or characterizing compounds that interact with their targets with moderate to low affinity or compounds that interact with targets that are not readily accessible.

The cell-based assay methods of the present invention have substantial advantages over current cell-based assays. These advantages derive from the use of sensitized cells in which the level or activity of at least one proliferation-required gene product (the target molecule) has been specifically reduced to the point where the presence or absence of its function becomes a rate-determining step for cellular proliferation. Bacterial, fungal, plant, or animal cells can all be used with the present method. Such sensitized cells become much more sensitive to compounds that are active against the affected target molecule. Thus, cell-based assays of the present invention are capable of detecting compounds exhibiting low or moderate potency against the target molecule of interest because such compounds are substantially more potent on sensitized cells than on non-sensitized cells. The effect may be such that a test compound may be two to several times more potent, at least 10 times more potent, at least 20 times more potent, at least 50 times more potent, at least 100 times more potent, at least 1000 times more potent, or even more than 1000 times more potent when tested on the sensitized cells as compared to the non-sensitized cells. The proliferation-required nucleic acids or polypeptides from Staphylococcus aureus, Salmonella typhimurium, Klebsiella pneumoniae, Pseudomonas aeruginosa, Enterococcus faecalis, Escherichia coli, Enterococcus faecalis, Haemophilus influenzae, Helicobacter pylori, or Salmonella typhi, or portions thereof, may be employed in any of the cell-based assays described herein. Similarly, homologous coding nucleic acids, homologous antisense nucleic acids, or homologous polypeptides or portions of the homologous nucleic acids or homologous polypeptides, may be employed in any of the cell-based assays described herein.

Due in part to the increased appearance of antibiotic resistance in pathogenic microorganisms and to the significant side-effects associated with some currently used antibiotics, novel antibiotics acting at new targets are highly sought after in the art. Yet, another limitation in the current art related to cell-based assays is the problem of repeatedly identifying hits against the same kinds of target molecules in the same limited set of biological pathways. This may occur when compounds acting at such new targets are discarded, ignored or fail to be detected because compounds acting at the “old” targets are encountered more frequently and are more potent than compounds acting at the new targets. As a result, the majority of antibiotics in use currently interact with a relatively small number of target molecules within an even more limited set of biological pathways.

The use of sensitized cells of the current invention provides a solution to the above problem in two ways. First, desired compounds acting at a target of interest, whether a new target or a previously known but poorly exploited target, can now be detected above the “noise” of compounds acting at the “old” targets due to the specific and substantial increase in potency of such desired compounds when tested on the sensitized cells of the current invention. Second, the methods used to sensitize cells to compounds acting at a target of interest may also sensitize these cells to compounds acting at other target molecules within the same biological pathway. For example, expression of an antisense molecule to a gene encoding a ribosomal protein is expected to sensitize the cell to compounds acting at that ribosomal protein and may also sensitize the cells to compounds acting at any of the ribosomal components (proteins or rRNA) or even to compounds acting at any target which is part of the protein synthesis pathway. Thus an important advantage of the present invention is the ability to reveal new targets and pathways that were previously not readily accessible to drug discovery methods.

Sensitized cells of the present invention are prepared by reducing the activity or level of a target molecule. The target molecule may be a gene product, such as an RNA or polypeptide produced from the proliferation-required nucleic acids from Staphylococcus aureus, Salmonella typhimurium, Klebsiella pneumoniae, Pseudomonas aeruginosa, Enterococcus faecalis, Escherichia coli, Enterococcus faecalis, Haemophilus influenzae, Helicobacter pylori, or Salmonella typhi (including a gene product produced from the nucleic acids of SEQ ID NOs.: 3796-3800, 3806-4860, 5916-10012, such as the polypeptides of SEQ ID NOs.: 3801-3805, 4861-5915, 10013-14110) or from homologous nucleic acids. For example, the target molecule may be one of the polypeptides of SEQ ID NOs. 3801-3805, 4861-5915, 10013-14110 or a homologous polypeptide. Alternatively, the target may be a gene product such as an RNA or polypeptide which is produced from a sequence within the same operon as the proliferation-required nucleic acids from Staphylococcus aureus, Salmonella typhimurium, Klebsiella pneumoniae, Pseudomonas aeruginosa, Enterococcus faecalis, Escherichia coli, Enterococcus faecalis, Haemophilus influenzae, Helicobacter pylori, or Salmonella typhi or from homologous nucleic acids. In addition, the target may be an RNA or polypeptide in the same biological pathway as the proliferation-required nucleic acids from Staphylococcus aureus, Salmonella typhimurium, Klebsiella pneumoniae, Pseudomonas aeruginosa, Enterococcus faecalis, Escherichia coli, Enterococcus faecalis, Haemophilus influenzae, Helicobacter pylori, or Salmonella typhi or from homologous nucleic acids. Such biological pathways include, but are not limited to, enzymatic, biochemical and metabolic pathways as well as pathways involved in the production of cellular structures such the cell wall.

Current methods employed in the arts of medicinal and combinatorial chemistries are able to make use of structure-activity relationship information derived from testing compounds in various biological assays including direct binding assays and cell-based assays. Occasionally compounds are directly identified in such assays that are sufficiently potent to be developed as drugs. More often, initial hit compounds exhibit moderate or low potency. Once a hit compound is identified with low or moderate potency, directed libraries of compounds are synthesized and tested in order to identify more potent leads. Generally these directed libraries are combinatorial chemical libraries consisting of compounds with structures related to the hit compound but containing systematic variations including additions, subtractions and substitutions of various structural features. When tested for activity against the target molecule, structural features are identified that either alone or in combination with other features enhance or reduce activity. This information is used to design subsequent directed libraries containing compounds with enhanced activity against the target molecule. After one or several iterations of this process, compounds with substantially increased activity against the target molecule are identified and may be further developed as drugs. This process is facilitated by use of the sensitized cells of the present invention since compounds acting at the selected targets exhibit increased potency in such cell-based assays, thus; more compounds can now be characterized providing more useful information than would be obtained otherwise.

Thus, it is now possible using cell-based assays of the present invention to identify or characterize compounds that previously would not have been readily identified or characterized including compounds that act at targets that previously were not readily exploited using cell-based assays. The process of evolving potent drug leads from initial hit compounds is also substantially improved by the cell-based assays of the present invention because, for the same number of test compounds, more structure-function relationship information is likely to be revealed.

The method of sensitizing a cell entails selecting a suitable gene or operon. A suitable gene or operon is one whose transcription and/or expression is required for the proliferation of the cell to be sensitized. The next step is to introduce into the cells to be sensitized, an antisense RNA capable of hybridizing to the suitable gene or operon or to the RNA encoded by the suitable gene or operon. Introduction of the antisense RNA can be in the form of a vector in which antisense RNA is produced under the control of an inducible promoter. The amount of antisense RNA produced is modulated by varying an inducer concentration to which the cell is exposed and thereby varying the activity of the promoter driving transcription of the antisense RNA. Thus, cells are sensitized by exposing them to an inducer concentration that results in a sub-lethal level of antisense RNA expression. The requisite maount of inducer may be derived empiracally by one of skill in the art.

In one embodiment of the cell-based assays, antisense nucleic acids complementary to the identified Staphylococcus aureus, Salmonella typhimurium, Klebsiella pneumoniae, Pseudomonas aeruginosa, Enterococcus faecalis, Escherichia coli, Enterococcus faecalis, Haemophilus influenzae, Helicobacter pylori, or Salmonella typhi nucleotide sequences or portions thereof (including antisense nucleic acids comprising a nucleotide sequence complementary to one of SEQ ID NOs.: 3796-3800, 3806-4860, 5916-10012, and the antisense nucleic acids of SEQ ID NOs.: 8-3795 or antisense nucleic acids comprising a nucleotide sequence complementary to portions of the foregoing nucleic acids thereof), antisense nucleic complementary to homologous coding nucleic acids or portions thereof or homologous antisense nucleic acids are used to inhibit the production of a proliferation-required protein. Vectors producing antisense RNA complementary to identified genes required for proliferation, or portions thereof, are used to limit the concentration of a proliferation-required protein without severely inhibiting growth. The proliferation-required protein may be one of the proteins of SEQ ID NOs.: 3801-3805, 4861-5915, 10013-14110 or a homologous polypeptide. To achieve that goal, a growth inhibition dose curve of inducer is calculated by plotting various doses of inducer against the corresponding growth inhibition caused by the antisense expression. From this curve, the concentration of inducer needed to achieve various percentages of antisense induced growth inhibition, from 1 to 100% can be determined.

A variety of different regulatable promoters may be used to produce the antisense nucleic acid. Transcription from the regulatable promoters may be modulated by controlling the activity of a transcription factor repressor which acts at the regulatable promoter. For example, if transcription is modulated by affecting the activity of a repressor, the choice of inducer to be used depends on the repressor/operator responsible for regulating transcription of the antisense nucleic acid. If the regulatable promoter comprises a T5 promoter fused to a xylO (xylose operator; e.g. derived from Staphylococcus xylosis (Schnappinger, D. et al., FEMS Microbiol. Let. 129: 121-128 (1995), the disclosure of which is incorporated herein by reference in its entirety) then transcription of the antisense nucleic acid may be regulated by a xylose repressor. The xylose repressor may be provided by ectoptic expression within an S. aureus cell of an exogenous xylose repressor gene, e.g. derived from S. xylosis DNA. In such cases transcription of antisense RNA from the promoter is inducible by adding xylose to the medium and the promoter is thus “xylose inducible.” Similarly, IPTG inducible promoters may be used. For example, the highest concentration of the inducer that does not reduce the growth rate significantly can be estimated from the curve. Cellular proliferation can be monitored by growth medium turbidity via OD measurements. In another example, the concentration of inducer that reduces growth by 25% can be predicted from the curve. In still another example, a concentration of inducer that reduces growth by 50% can be calculated. Additional parameters such as colony forming units (cfu) can be used to measure cellular viability.

Cells to be assayed are exposed to the above-determined concentrations of inducer. The presence of the inducer at this sub-lethal concentration reduces the amount of the proliferation required gene product to a sub-optimal amount in the cell that will still support growth. Cells grown in the presence of this concentration of inducer are therefore specifically more sensitive to inhibitors of the proliferation-required protein or RNA of interest or to inhibitors of proteins or RNAs in the same biological pathway as the proliferation-required protein or RNA of interest but not to inhibitors of unrelated proteins or RNAs.

Cells pretreated with sub-inhibitory concentrations of inducer and thus containing a reduced amount of proliferation-required target gene product are then used to screen for compounds that reduce cell growth. The sub-lethal concentration of inducer may be any concentration consistent with the intended use of the assay to identify candidate compounds to which the cells are more sensitive. For example, the sub-lethal concentration of the inducer may be such that growth inhibition is at least about 5%, at least about 8%, at least about 10%, at least about 20%, at least about 30%, at least about 40%, at least about 50%, at least about 60% at least about 75%, or more. Cells which are pre-sensitized using the preceding method are more sensitive to inhibitors of the target protein because these cells contain less target protein to inhibit than do wild-type cells.

It will be appreciated that the above cell-based assays may be performed using antisense nucleic acids comprising a nucleotide sequence complementary to any of the proliferation-required nucleic acids from Staphylococcus aureus, Salmonella typhimurium, Klebsiella pneumoniae, Pseudomonas aeruginosa, Enterococcus faecalis, Escherichia coli, Enterococcus faecalis, Haemophilus influenzae, Helicobacter pylori, or Salmonella typhi, or portions thereof, antisense nucleic acids complementary to homologous coding nucleic acids or portions thereof or homologous antisense nucleic acids. In this way, the level or activity of a target, such as any of the proliferation-required polypeptides from Staphylococcus aureus, Salmonella typhimurium, Klebsiella pneumoniae, Pseudomonas aeruginosa, Enterococcus faecalis, Escherichia coli, Enterococcus faecalis, Haemophilus influenzae, Helicobacter pylori, or Salmonella typhi, or homologous polypeptides.

In another embodiment of the cell-based assays of the present invention, the level or activity of a proliferation required gene product is reduced using a mutation, such as a temperature sensitive mutation, in the gene encoding a gene product required for proliferation and an antisense nucleic acid comprising a nucleotide sequence complementary to the gene encoding the gene product required for proliferation or a portion thereof. Growing the cells at an intermediate temperature between the permissive and restrictive temperatures of the temperature sensitive mutant where the mutation is in a proliferation-required gene produces cells with reduced activity of the proliferation-required gene product. The antisense RNA complementary to the proliferation-required sequence further reduces the activity of the proliferation required gene product. Drugs that may not have been found using either the temperature sensitive mutation or the antisense nucleic acid alone may be identified by determining whether cells in which transcription of the antisense nucleic acid has been induced and which are grown at a temperature between the permissive temperature and the restrictive temperature are substantially more sensitive to a test compound than cells in which expression of the antisense nucleic acid has not been induced and which are grown at a permissive temperature. Also drugs found previously from either the antisense nucleic acid alone or the temperature sensitive mutation alone may have a different sensitivity profile when used in cells combining the two approaches, and that sensitivity profile may indicate a more specific action of the drug in inhibiting one or more activities of the gene product.

Temperature sensitive mutations may be located at different sites within the gene and correspond to different domains of the protein. For example, the dnaB gene of Escherichia coli encodes the replication fork DNA helicase. DnaB has several domains, including domains for oligomerization, ATP hydrolysis, DNA binding, interaction with primase, interaction with DnaC, and interaction with DnaA [(Biswas, E. E. and Biswas, S. B. 1999. Mechanism and DnaB helicase of Escherichia coli: structural domains involved in ATP hydrolysis, DNA binding, and oligomerization. Biochem. 38:10919-10928; Hiasa, H. and Marians, K. J. 1999. Initiation of bidirectional replication at the chromosomal origin is directed by the interaction between helicase and primase. J. Biol. Chem. 274:27244-27248; San Martin, C., Radermacher, M., Wolpensinger, B., Engel, A., Miles, C. S., Dixon, N. E., and Carazo, J. M. 1998. Three-dimensional reconstructions from cryoelectron microscopy images reveal an intimate complex between helicase DnaB and its loading partner DnaC. Structure 6:501-9; Sutton, M. D., Carr, K. M., Vicente, M., and Kaguni, J. M. 1998. Escherichia coli DnaA protein. The N-terminal domain and loading of DnaB helicase at the E. coli chromosomal origin. J. Biol. Chem. 273:34255-62.), the disclosures of which are incorporated herein by reference in their entireties]. Temperature sensitive mutations in different domains of DnaB confer different phenotypes at the restrictive temperature, which include either an abrupt stop or slow stop in DNA replication with or without DNA breakdown (Wechsler, J. A. and Gross, J. D. 1971. Escherichia coli mutants temperature-sensitive for DNA synthesis. Mol. Gen. Genetics 113:273-284, the disclosure of which is incorporated herein by reference in its entirety) and termination of growth or cell death. Combining the use of temperature sensitive mutations in the dnaB gene that cause cell death at the restrictive temperature with an antisense to the dnaB gene could lead to the discovery of very specific and effective inhibitors of one or a subset of activities exhibited by DnaB.

It will be appreciated that the above method may be performed with any mutation which reduces but does not eliminate the activity or level of the gene product which is required for proliferation.

It will be appreciated that the above cell-based assays may be performed using mutations in, such as temperature sensitive mutations, and antisense nucleic acids comprising a nucleotide sequence complementary to any of the genes encoding proliferation-required gene products from from Staphylococcus aureus, Salmonella typhimurium, Klebsiella pneumoniae, Pseudomonas aeruginosa, Enterococcus faecalis, Escherichia coli, Enterococcus faecalis, Haemophilus influenzae, Helicobacter pylori, or Salmonella typhi, or portions thereof (including the nucleic acids of SEQ ID NOs.: 3796-3800, 3806-4860, 5916-10012), mutations in and antisense nucleic acids complementary to homologous coding nucleic acids or portions thereof or homologous antisense nucleic acids. In this way, the level or activity of a target, such as any of the proliferation-required polypeptides from Staphylococcus aureus, Salmonella typhimurium, Klebsiella pneumoniae, Pseudomonas aeruginosa, Enterococcus faecalis, Escherichia coli, Enterococcus faecalis, Haemophilus influenzae, Helicobacter pylori, or Salmonella typhi (including the polypeptides of SEQ ID NOs.: 3801-3805, 4861-5915, 10013-14110), or homologous polypeptides may be reduced.

When screening for antimicrobial agents against a gene product required for proliferation, growth inhibition of cells containing a limiting amount of that proliferation-required gene product can be assayed. Growth inhibition can be measured by directly comparing the amount of growth, measured by the optical density of the growth medium, between an experimental sample and a control sample. Alternative methods for assaying cell proliferation include measuring green fluorescent protein (GFP) reporter construct emissions, various enzymatic activity assays, and other methods well known in the art.

It will be appreciated that the above method may be performed in solid phase, liquid phase or a combination of the two. For example, cells grown on nutrient agar containing the inducer of the antisense construct may be exposed to compounds spotted onto the agar surface. If desired, the cells may be grown on agar containing varying concentrations of the inducer. A compound's effect may be judged from the diameter of the resulting killing zone, the area around the compound application point in which cells do not grow. Multiple compounds may be transferred to agar plates and simultaneously tested using automated and semi-automated equipment including but not restricted to multi-channel pipettes (for example the Beckman Multimek) and multi-channel spotters (for example the Genomic Solutions Flexys). In this way multiple plates and thousands to millions of compounds may be tested per day.

The compounds may also be tested entirely in liquid phase using microtiter plates as described below. Liquid phase screening may be performed in microtiter plates containing 96, 384, 1536 or more wells per microtiter plate to screen multiple plates and thousands to millions of compounds per day. Automated and semi-automated equipment may be used for addition of reagents (for example cells and compounds) and determination of cell density.

### Example 9

### Cell-based Assay Using Antisense Complementary to Genes Encoding Ribosomal Proteins

The effectiveness of the above cell-based assay was validated using constructs transribing antisense RNA to the proliferation required E. coli genes rplL, rplJ, and rplW encoding ribosomal proteins L7/L12, L10 and L23 respectively. These proteins are essential components of the protein synthesis apparatus of the cell and as such are required for proliferation. These constructs were used to test the effect of antisense transcription on cell sensitivity to antibiotics known to bind to the ribosome and thereby inhibit protein synthesis. Constructs transcribing antisense RNA to several other genes (elaD, visC, yohH, and atpE/B), the products of which are not involved in protein synthesis were used for comparison.

First, pLex5BA (Krause et al., J. Mol. Biol. 274: 365 (1997), the disclosure of which is incorporated herein by reference in its entirety) vectors containing antisense constructs to either rplW or to elaD were introduced into separate E. coli cell populations. Vector introduction is a technique well known to those of ordinary skill in the art. The vectors of this example contain IPTG inducible promoters that drive the transcription of the antisense RNA in the presence of the inducer. However, those skilled in the art will appreciate that other inducible promoters may also be used. Suitable vectors are also well known in the art. Antisense clones to genes encoding different ribosomal proteins or to genes encoding proteins that are not involved in protein synthesis were utilized to test the effect of antisense transcription on cell sensitivity to the antibiotics known to bind to ribosomal proteins and inhibit protein synthesis. Antisense nucleic acids comprising a nucleotide sequence complementarty to the elaD, atpB&atpE, visC and yohH genes are referred to as AS-elaD, AS-atpB/E, AS-visC, AS-yohH respectively. These genes are not known to be involved in protein synthesis. Antisense nucleic acids to the rplL, rplL&rplJ and rplW genes are referred to as AS-rplL, AS-rplL/J, and AS-rplW respectively. These genes encode ribosomal proteins L7/L12 (rplL) L10 (rplJ) and L23 (rplW). Vectors containing these antisense nucleic acids were introduced into separate E. coli cell populations.

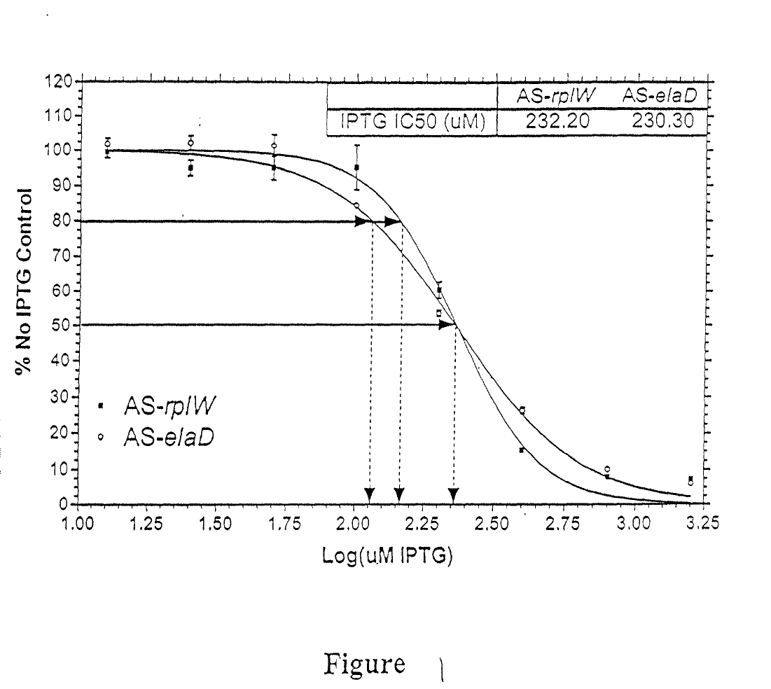

The cell populations containing vectors producing AS-elaD or AS-rplW were exposed to a range of IPTG concentrations in liquid medium to obtain the growth inhibitory dose curve for each clone (FIG. 1). First, seed cultures were grown to a particular turbidity measured by the optical density (OD) of the growth solution. The OD of the solution is directly related to the number of bacterial cells contained therein. Subsequently, sixteen 200 μl liquid medium cultures were grown in a 96 well microtiter plate at 37° C. with a range of IPTG concentrations in duplicate two-fold serial dilutions from 1600 uM to 12.5 μM (final concentration). Additionally, control cells were grown in duplicate without IPTG. These cultures were started from an inoculum of equal amounts of cells derived from the same initial seed culture of a clone of interest. The cells were grown for up to 15 hours and the extent of growth was determined by measuring the optical density of the cultures at 600 nm. When the control culture reached mid-log phase the percent growth (relative to the control culture) for each of the IPTG containing cultures was plotted against the log concentrations of IPTG to produce a growth inhibitory dose response curve for the IPTG. The concentration of IPTG that inhibits cell growth to 50% (IC₅₀) as compared to the 0 mM IPTG control (0% growth inhibition) was then calculated from the curve. Under these conditions, an amount of antisense RNA was produced that reduced the expression levels of rplW or elaD to a degree such that growth of cells containing their respective antisense vectors was inhibited by 50%.

Alternative methods of measuring growth are also contemplated. Examples of these methods include measurements of proteins, the expression of which is engineered into the cells being tested and can readily be measured. Examples of such proteins include green fluorescent protein (GFP), luciferase, and various enzymes.

Cells were pretreated with the selected concentration of IPTG and then used to test the sensitivity of cell populations to tetracycline, erythromycin and other known protein synthesis inhibitors. FIG. 1 is an IPTG dose response curve in E. coli transformed with an IPTG-inducible plasmid containing either an antisense clone to the E. coli rplW gene (AS-rplW) which encodes ribosomal protein L23 which is required for protein synthesis and essential for cell proliferation, or an antisense clone to the elaD (AS-elaD) gene which is not known to be involved in protein synthesis.

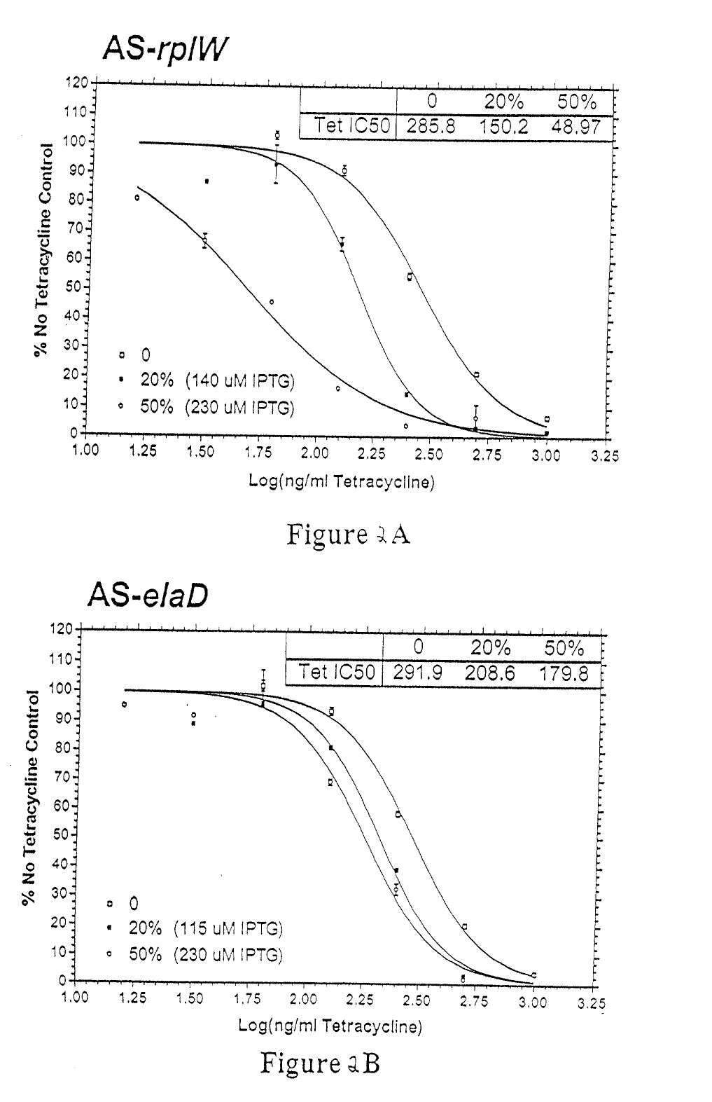

An example of a tetracycline dose response curve is shown in FIGS. 2A and 2B for the rplW and elaD genes, respectively. Cells were grown to log phase and then diluted into medium alone or medium containing IPTG at concentrations which give 20% and 50% growth inhibition as determined by IPTG dose response curves. After 2.5 hours, the cells were diluted to a final OD₆₀₀ of 0.002 into 96 well plates containing (1) +/−IPTG at the same concentrations used for the 2.5 hour pre-incubation; and (2) serial two-fold dilutions of tetracycline such that the final concentrations of tetracycline range from 1 μg/ml to 15.6 ng/ml and 0 μg/ml. The 96 well plates were incubated at 37° C. and the OD₆₀₀ was read by a plate reader every 5 minutes for up to 15 hours. For each IPTG concentration and the no IPTG control, tetracycline dose response curves were determined when the control (absence of tetracycline) reached 0.1 OD₆₀₀.

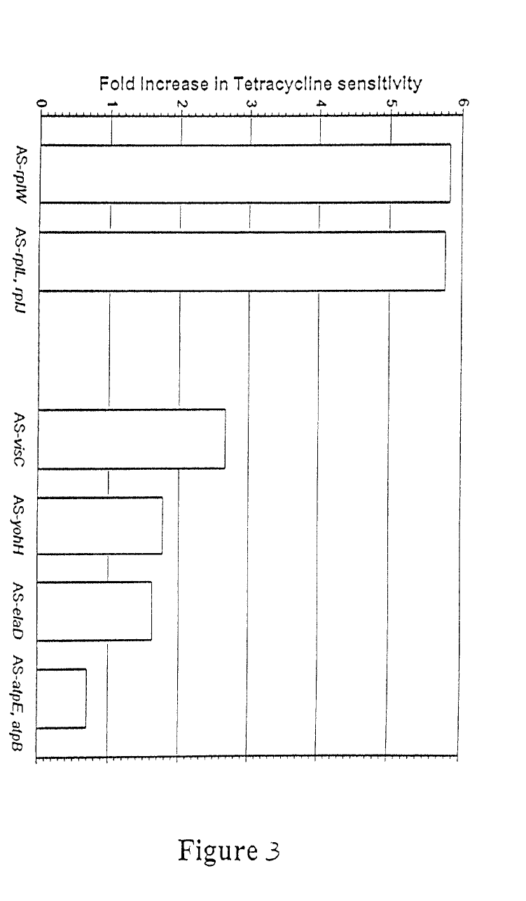

To compare tetracycline sensitivity with and without IPTG, tetracycline IC₅₀, were determined from the dose response curves (FIGS. 3A-B). Cells transcribing antisense nucleic acids AS-rplL or AS-rplW to genes encoding ribosomal proteins L7/L 12 and L23 respectively showed increased sensitivity to tetracycline (FIG. 2A) as compared to cells with reduced levels of the elaD gene product (AS-elaD) (FIG. 2B). FIG. 3 shows a summary bar chart in which the ratios of tetracycline IC₅₀s determined in the presence of IPTG which gives 50% growth inhibition versus tetracycline IC50S determined without IPTG (fold increase in tetracycline sensitivity) were plotted. Cells with reduced levels of either L7/L 12 (encoded by genes rplL, rplJ) or L23 (encoded by the rplW gene) showed increased sensitivity to tetracycline (FIG. 3). Cells expressing antisense to genes not known to be involved in protein synthesis (AS-atpB/E, AS-visC, AS-elaD, AS-yohH) did not show the same increased sensitivity to tetracycline, validating the specificity of this assay (FIG. 3).

In addition to the above, it has been observed in initial experiments that clones transcribing antisense RNA to genes involved in protein synthesis (including genes encoding ribosomal proteins L7/L12 & L10, L7/L12 alone, L22, and L18, as well as genes encoding rRNA and Elongation Factor G) have increased sensitivity to the macrolide, erythromycin, whereas clones transcribing antisense to the non-protein synthesis genes elaD, atpB/E and visC do not. Furthermore, the clone transcribing antisense to rplL and rplJ (AS-rplL/J) does not show increased sensitivity to nalidixic acid and ofloxacin, antibiotics which do not inhibit protein synthesis.

The results with the ribosomal protein genes rplL, rplJ, and rplW as well as the initial results using various other antisense clones and antibiotics show that limiting the concentration of an antibiotic target makes cells more sensitive to the antimicrobial agents that specifically interact with that protein. The results also show that these cells are sensitized to antimicrobial agents that inhibit the overall function in which the protein target is involved but are not sensitized to antimicrobial agents that inhibit other functions. It will be appreciated that the cell-based assays described above may be implemented using the Staphylococcus aureus, Salmonella typhimurium, Klebsiella pneumoniae, Pseudomonas aeruginosa, Enterococcus faecalis, Escherichia coli, Enterococcus faecalis, Haemophilus influenzae, Helicobacter pylori, or Salmonella typhi antisense nucleotide sequences which inhibit the activity of genes required for proliferation described herein (including the antisense nucleic acids of SEQ ID NOs.: 8-3795) or antisense nucleic acids comprising nucleotide sequences which are complementary to the sequences of SEQ ID NOs.: 3796-3800, 3806-4860, 5916-10012 or portions thereof.

It will be appreciated that the above cell-based assays may be performed using antisense nucleic acids complementary to any of the proliferation-required nucleic acids from Staphylococcus aureus, Salmonella typhimurium, Klebsiella pneumoniae, Pseudomonas aeruginosa, Enterococcus faecalis, Escherichia coli, Enterococcus faecalis, Haemophilus influenzae, Helicobacter pylori, or Salmonella typhi, or portions thereof, antisense nucleic acids complementary to homologous coding nucleic acids or portions thereof, or homologous antisense nucleic acids. In this way, the level or activity of a target, such as any of the proliferation-required polypeptides from Staphylococcus aureus, Salmonella typhimurium, Klebsiella pneumoniae, Pseudomonas aeruginosa, Enterococcus faecalis, Escherichia coli, Enterococcus faecalis, Haemophilus influenzae, Helicobacter pylori, or Salmonella typhi, or homologous polypeptides may be reduced.

The cell-based assay described above may also be used to identify the biological pathway in which a proliferation-required nucleic acid or its gene product lies. In such methods, cells transcribing a sub-lethal level of antisense to a target proliferation-required nucleic acid and control cells in which transcription of the antisense has not been induced are contacted with a panel of antibiotics known to act in various pathways. If the antibiotic acts in the pathway in which the target proliferation-required nucleic acid or its gene product lies, cells in which transcription of the antisense has been induced will be more sensitive to the antibiotic than cells in which expression of the antisense has not been induced.

As a control, the results of the assay may be confirmed by contacting a panel of cells transcribing antisense nucleic acids to many different proliferation-required genes including the target proliferation-required gene. If the antibiotic is acting specifically, heightened sensitivity to the antibiotic will be observed only in the cells transcribing antisense to a target proliferation-required gene (or cells expressing antisense to other proliferation-required genes in the same pathway as the target proliferation-required gene) but will not be observed generally in all cells expressing antisense to proliferation-required genes.

It will be appreciated that the above cell-based assays may be performed using antisense nucleic acids complementary to any of the proliferation-required nucleic acids from Staphylococcus aureus, Salmonella typhimurium, Klebsiella pneumoniae, Pseudomonas aeruginosa, Enterococcus faecalis, Escherichia coli, Enterococcus faecalis, Haemophilus influenzae, Helicobacter pylori, or Salmonella typhi , (including antisense nucleic acids complementary to SEQ ID NOs: 3796-3800, 3806-4860, 5916-10012, or the antisense nucleic acids of SEQ ID NOs.: 8-3795) or portions thereof, antisense nucleic acids comprising nucleotide sequences complementary to homologous coding nucleic acids or portions thereof, or homologous antisense nucleic acids In this way, the level or activity of a target, such as any of the proliferation-required polypeptides from Staphylococcus aureus, Salmonella typhimurium, Klebsiella pneumoniae, Pseudomonas aeruginosa, Enterococcus faecalis, Escherichia coli, Enterococcus faecalis, Haemophilus influenzae, Helicobacter pylori, or Salmonella typhi (including the polypeptides of SEQ ID NOs.: 3801-3805, 4861-5915, 10013-14110), or homologous polypeptides may be reduced.

Similarly, the above method may be used to determine the pathway on which a test compound, such as a test antibiotic acts. A panel of cells, each of which transcribes an antisense to a proliferation-required nucleic acid in a known pathway, is contacted with a compound for which it is desired to determine the pathway on which it acts. The sensitivity of the panel of cells to the test compound is determined in cells in which transcription of the antisense has been induced and in control cells in which expression of the antisense has not been induced. If the test compound acts on the pathway on which an antisense nucleic acid acts, cells in which expression of the antisense has been induced will be more sensitive to the compound than cells in which expression of the antisense has not been induced. In addition, control cells in which expression of antisense to proliferation-required genes in other pathways has been induced will not exhibit heightened sensitivity to the compound. In this way, the pathway on which the test compound acts may be determined.

It will be appreciated that the above cell-based assays may be performed using antisense nucleic acids comprising nucleotide sequences complementary to any of the proliferation-required nucleic acids from Staphylococcus aureus, Salmonella typhimurium, Klebsiella pneumoniae, Pseudomonas aeruginosa, Enterococcus faecalis, Escherichia coli, Enterococcus faecalis, Haemophilus influenzae, Helicobacter pylori, or Salmonella typhi (including antisense nucleic acids complementary to SEQ ID NOs: 3796-3800, 3806-4860, 5916-10012, such as the antisense nucleic acids of SEQ ID NOs.: 8-3795) or portions thereof, antisense nucleic acids complementary to homologous coding nucleic acids or portions thereof, or homologous antisense nucleic acids In this way, the level or activity of a target, such as any of the proliferation-required polypeptides from Staphylococcus aureus, Salmonella typhimurium, Klebsiella pneumoniae, Pseudomonas aeruginosa, Enterococcus faecalis, Escherichia coli, Enterococcus faecalis, Haemophilus influenzae, Helicobacter pylori, or Salmonella typhi (including the polypeptides of SEQ ID NOs.: 3801-3805, 4861-5915, 10013-14110) or homologous polypeptides may be reduced.

The Example below provides one method for performing such assays.

### Example 10

### Identification of the Pathway in which a Proliferation-Required Gene Lies or the Pathway on which an Antibiotic Acts

A. Preparation of Bacterial Stocks for Assay

To provide a consistent source of cells to screen, frozen stocks of host bacteria containing the desired antisense construct are prepared using standard microbiological techniques. For example, a single clone of the microorganism can be isolated by streaking out a sample of the original stock onto an agar plate containing nutrients for cell growth and an antibiotic for which the antisense construct contains a selectable marker which confers resistance. After overnight growth an isolated colony is picked from the plate with a sterile needle and transferred to an appropriate liquid growth medium containing the antibiotic required for maintenance of the plasmid. The cells are incubated at 30° C. to 37° C. with vigorous shaking for 4 to 6 hours to yield a culture in exponential growth. Sterile glycerol is added to 15% (volume to volume) and 100 μL to 500 μL aliquots are distributed into sterile cryotubes, snap frozen in liquid nitrogen, and stored at −80° C. for future assays.

B. Growth of Bacteria for Use in the Assay

A day prior to an assay, a stock vial is removed from the freezer, rapidly thawed (37° C. water bath) and a loop of culture is streaked out on an agar plate containing nutrients for cell growth and an antibiotic to which the selectable marker of the antisense construct confers resistance. After overnight growth at 37° C., ten randomly chosen, isolated colonies are transferred from the plate (sterile inoculum loop) to a sterile tube containing 5 mL of LB medium containing the antibiotic to which the antisense vector confers resistance. After vigorous mixing to form a homogeneous cell suspension, the optical density of the suspension is measured at 600 rm (OD₆₀₀) and if necessary an aliquot of the suspension is diluted into a second tube of 5 mL, sterile, LB medium plus antibiotic to achieve an OD₆₀₀≦0.02 absorbance units. The culture is then incubated at 37° C. for 1-2 hrs with shaking until the OD₆₀₀ reaches OD 0.2-0.3. At this point the cells are ready to be used in the assay.

C. Selection of Media to be Used in Assay

Two-fold dilution series of the inducer are generated in culture media containing the appropriate antibiotic for maintenance of the antisense construct. Several media are tested side by side and three to four wells are used to evaluate the effects of the inducer at each concentration in each media. For example, LB broth, TBD broth and Muller-Hinton media may be tested with the inducer xylose at the following concentrations, 5 mM, 10 mM, 20 mM, 40 mM, 80 mM, 120 mM and 160 mM. Equal volumes of test media-inducer and cells are added to the wells of a 384 well microtiter plate and mixed. The cells are prepared as described above and diluted 1:100 in the appropriate media containing the test antibiotic immediately prior to addition to the microtiter plate wells. For a control, cells are also added to several wells of each media that do not contain inducer, for example 0 mM xylose. Cell growth is monitored continuously by incubation at 37° C. in a microtiter plate reader monitoring the OD₆₀₀ of the wells over an 18-hour period. The percent inhibition of growth produced by each concentration of inducer is calculated by comparing the rates of logarithmic growth against that exhibited by cells growing in medium without inducer. The medium yielding greatest sensitivity to inducer is selected for use in the assays described below.

D. Measurement of Test Antibiotic Sensitivity in the Absence of Antisense Construct Induction

Two-fold dilution series of antibiotics of known mechanism of action are generated in the culture medium selected for further assay development that has been supplemented with the antibiotic used to maintain the construct. A panel of test antibiotics known to act on different pathways is tested side by side with three to four wells being used to evaluate the effect of a test antibiotic on cell growth at each concentration. Equal volumes of test antibiotic and cells are added to the wells of a 384 well microtiter plate and mixed. Cells are prepared as described above using the medium selected for assay development supplemented with the antibiotic required to maintain the antisense construct and are diluted 1:100 in identical medium immediately prior to addition to the microtiter plate wells. For a control, cells are also added to several wells that lack antibiotic, but contain the solvent used to dissolve the antibiotics. Cell growth is monitored continuously by incubation at 37° C. in a microtiter plate reader monitoring the OD₆₀₀ of the wells over an 18-hour period. The percent inhibition of growth produced by each concentration of antibiotic is calculated by comparing the rates of logarithmic growth against that exhibited by cells growing in medium without antibiotic. A plot of percent inhibition against log[antibiotic concentration] allows extrapolation of an IC₅₀ value for each antibiotic.

E. Measurement of Test Antibiotic Sensitivity in the Presence of Antisense Construct Inducer

The culture medium selected for use in the assay is supplemented with inducer at concentrations shown to inhibit cell growth by 50% and 80% as described above, as well as the antibiotic used to maintain the construct. Two-fold dilution series of the panel of test antibiotics used above are generated in each of these media. Several antibiotics are tested side by side in each medium with three to four wells being used to evaluate the effects of an antibiotic on cell growth at each concentration. Equal volumes of test antibiotic and cells are added to the wells of a 384 well microtiter plate and mixed. Cells are prepared as described above using the medium selected for use in the assay supplemented with the antibiotic required to maintain the antisense construct. The cells are diluted 1:100 into two 50 mL aliquots of identical medium containing concentrations of inducer that have been shown to inhibit cell growth by 50% and 80% respectively and incubated at 37° C. with shaking for 2.5 hours. Immediately prior to addition to the microtiter plate wells, the cultures are adjusted to an appropriate OD₆₀₀ (typically 0.002) by dilution into warm (37° C.) sterile medium supplemented with identical concentrations of the inducer and antibiotic used to maintain the antisense construct. For a control, cells are also added to several wells that contain solvent used to dissolve test antibiotics but which contain no antibiotic. Cell growth is monitored continuously by incubation at 37° C. in a microtiter plate reader monitoring the OD₆₀₀ of the wells over an 18-hour period. The percent inhibition of growth produced by each concentration of antibiotic is calculated by comparing the rates of logarithmic growth against that exhibited by cells growing in medium without antibiotic. A plot of percent inhibition against log[antibiotic concentration] allows extrapolation of an IC₅₀ value for each antibiotic.

F. Determining the Specificity of the Test Antibiotics

A comparison of the IC₅₀s generated by antibiotics of known mechanism of action under antisense induced and non-induced conditions allows the pathway in which a proliferation-required nucleic acid lies to be identified. If cells expressing an antisense nucleic acid comprising a nucleotide sequence complementary to a proliferation-required gene are selectively sensitive to an antibiotic acting via a particular pathway, then the gene against which the antisense acts is involved in the pathway on which the antibiotic acts.

G. Identification of Pathway in which a Test Antibiotic Acts

As discussed above, the cell-based assay may also be used to determine the pathway against which a test antibiotic acts. In such an analysis, the pathways against which each member of a panel of antisense nucleic acids acts are identified as described above. A panel of cells, each containing an inducible vector which transcribes an antisense nucleic acid comprising a nucleotide sequence complementary to a gene in a known proliferation-required pathway, is contacted with a test antibiotic for which it is desired to determine the pathway on which it acts under inducing and non-inducing conditions. If heightened sensitivity is observed in induced cells transcribing antisense complementary to a gene in a particular pathway but not in induced cells transcribing antisense nucleic acids comprising nucleotide sequences complementary to genes in other pathways, then the test antibiotic acts against the pathway for which heightened sensitivity was observed.

One skilled in the art will appreciate that further optimization of the assay conditions, such as the concentration of inducer used to induce antisense transcription and/or the growth conditions used for the assay (for example incubation temperature and medium components) may further increase the selectivity and/or magnitude of the antibiotic sensitization exhibited.

It will be appreciated that the above cell-based assays may be performed using antisense nucleic acids comprising nucleotide sequences complementary to any of the proliferation-required nucleic acids from Staphylococcus aureus, Salmonella typhimurium, Klebsiella pneumoniae, Pseudomonas aeruginosa, Enterococcus faecalis, Escherichia coli, Enterococcus faecalis, Haemophilus influenzae, Helicobacter pylori, or Salmonella typhi, (including antisense nucleic acids comprising nucleotide sequences complemenatary to SEQ ID NOs: 3796-3800, 3806-4860, 5916-10012, such as the antisense nucleic acids of SEQ ID NOs.: 8-3795) or portions thereof, antisense nucleic acids complementary to homologous coding nucleic acids or portions thereof or homologous antisense nucleic acids In this way, the level or activity of a target, such as any of the proliferation-required polypeptides from Staphylococcus aureus, Salmonella typhimurium, Klebsiella pneumoniae, Pseudomonas aeruginosa, Enterococcus faecalis, Escherichia coli, Enterococcus faecalis, Haemophilus influenzae, Helicobacter pylori, or Salmonella typhi (including the polypeptides of SEQ ID NOs.: 3801-3805, 4861-5915, 10013-14110), or homologous polypeptides may be reduced.

The following example confirms the effectiveness of the methods described above.

### Example 11

### Identification of the Biological Pathway in which a Proliferation-Required Gene Lies

The effectiveness of the above assays was validated using proliferation-required genes from E. coli which were identified using procedures similar to those described above. Antibiotics of various chemical classes and modes of action were purchased from Sigma Chemicals (St. Louis, Mo.). Stock solutions were prepared by dissolving each antibiotic in an appropriate aqueous solution based on information provided by the manufacturer. The final working solution of each antibiotic contained no more than 0.2% (w/v) of any organic solvent. To determine their potency against a bacterial strain engineered for transcription of an antisense comprising a nucleotide sequence complementary to a proliferation-required 50S ribosomal protein, each antibiotic was serially diluted two- or three- fold in growth medium supplemented with the appropriate antibiotic for maintenance of the antisense construct. At least ten dilutions were prepared for each antibiotic. 25 μL aliquots of each dilution were transferred to discrete wells of a 384-well microplate (the assay plate) using a multi-channel pipette. Quadruplicate wells were used for each dilution of an antibiotic under each treatment condition (plus and minus inducer). Each assay plate contained twenty wells for cell growth controls (growth medium replacing antibiotic), ten wells for each treatment (plus and minus inducer, in this example IPTG). Assay plates were usually divided into the two treatments: half the plate containing induced cells and an appropriate concentrations of inducer (in this example IPTG) to maintain the state of induction, the other half containing non-induced cells in the absence of IPTG.

Cells for the assay were prepared as follows. Bacterial cells containing a construct, from which transcription of antisense nucleic acid comprising a nucleotide sequence complementary to rplL and rplJ (AS-rplL/J), which encode proliferation-required 50S ribosomal subunit proteins, is inducible in the presence of IPTG, were grown into exponential growth (OD₆₀₀ 0.2 to 0.3) and then diluted 1:100 into fresh medium containing either 400 μM or 0 μM inducer (IPTG). These cultures were incubated at 37° C. for 2.5 hr. After a 2.5 hr incubation, induced and non-induced cells were respectively diluted into an assay medium at a final OD₆₀₀ value of 0.0004. The medium contained an appropriate concentration of the antibiotic for the maintenance of the antisense construct. In addition, the medium used to dilute induced cells was supplemented with 800 μM IPTG so that addition to the assay plate would result in a final IPTG concentration of 400 μM. Induced and non-induced cell suspensions were dispensed (25 μl/well) into the appropriate wells of the assay plate as discussed previously. The plate was then loaded into a plate reader, incubated at constant temperature, and cell growth was monitored in each well by the measurement of light scattering at 595 nm. Growth was monitored every 5 minutes until the cell culture attained a stationary growth phase. For each concentration of antibiotic, a percentage inhibition of growth was calculated at the time point corresponding to mid-exponential growth for the associated control wells (no antibiotic, plus or minus IPTG). For each antibiotic and condition (plus or minus IPTG), a plot of percent inhibition versus log of antibiotic concentration was generated and the IC₅₀ determined. A comparison of the IC₅₀ for each antibiotic in the presence and absence of IPTG revealed whether induction of the antisense construct sensitized the cell to the mechanism of action exhibited by the antibiotic. Cells which exhibited a statistically significant decrease in the IC₅₀ value in the presence of inducer were considered to have an increased sensitivity to the test antibiotic.

The results are provided in the table below, which lists the classes and names of the antibiotics used in the analysis, the targets of the antibiotics, the IC₅₀ in the absence of IPTG, the IC₅₀ in the presence of IPTG, the concentration units for the IC₅₀s, the fold increase in IC₅₀ in the presence of IPTG, and whether increased sensitivity was observed in the presence of IPTG.

TABLE III
Effect of Expression of Antisense RINA to rylL and rplJ on Antibiotic Sensitivity

| | | | | | Fold | |
|---|---|---|---|---|---|---|
| | | IC₅₀ | IC₅₀ | Conc. | Increase in | Sensitivity |
| ANTIBIOTIC CLASS /Names | TARGET | (−IPTG) | (+IPTG) | Unit | Sensitivity | Increased? |
| PROTEIN SYNTHESIS INHIBITOR | | | | | | |
| AMINOGLYCOSIDES | | | | | | |
| Gentamicin | 30S ribosome function | 2715 | 19.19 | ng/ml | 141 | Yes |
| Streptomycin | 30S ribosome function | 11280 | 161 | ng/ml | 70 | Yes |
| Spectinomycin | 30S ribosome function | 18050 | <156 | ng/ml | Yes | |
| Tobramycin | 30S ribosome function | 3594 | 70.58 | ng/ml | 51 | Yes |
| MACROLIDES | 50S ribosome function | 7467 | 187 | ng/ml | 40 | Yes |
| Erythromycin | | | | | | |
| AROMATIC POYKETIDES | | | | | | |
| Tetracycline | 30S ribosome function | 199.7 | 1.83 | ng/ml | 109 | Yes |
| Minocycline | 30S ribosome function | 668.4 | 3.897 | ng/ml | 172 | Yes |
| Doxycycline | 30S ribosome function | 413.1 | 27.81 | ng/ml | 15 | Yes |
| OTHER PROTEIN SYNTHESIS | | | | | | |
| INHIBITORS | | | | | | |
| Fusidic acid | Elongation Factor G function | 59990 | 641 | ng/ml | 94 | Yes |
| Chloramphenicol | 30S ribosome function | 465.4 | 1.516 | ng/ml | 307 | Yes |
| Lincomycin | 50S ribosome function | 47150 | 324.2 | ng/ml | 145 | Yes |
| OTHER ANTIBIOTIC MECHANISMS | | | | | | |
| B-LACTAMS | | | | | | |
| Cefoxitin | Cell wall biosynthesis | 2782 | 2484 | ng/ml | 1 | No |
| Cefotaxime | Cell wall biosynthesis | 24.3 | 24.16 | ng/ml | 1 | No |
| DNA SYNTHESIS INHIBITORS | | | | | | |
| Nalidixic acid | DNA Gyrase activity | 6973 | 6025 | ng/ml | 1 | No |
| Ofloxacin | DNA Gyrase activity | 49.61 | 45.89 | ng/ml | 1 | No |
| OTHER | | | | | | |
| Bacitracin | Cell membrane function | 4077 | 4677 | mg/ml | 1 | No |
| | Dihydrofolate Reductase | | | | | |
| Trimethoprim | activity | 128.9 | 181.97 | ng/ml | 1 | No |
| Vancomycin | Cell wall biosynthesis | 145400 | 72550 | ng/ml | 2 | No |

The above results demonstrate that induction of an antisense RNA complementary to genes encoding 50S ribosomal subunit proteins results in a selective and highly significant sensitization of cells to antibiotics that inhibit ribosomal function and protein synthesis. The above results further demonstrate that induction of an antisense to an essential gene sensitizes a cell or microorganism to compounds that interfere with that gene product's biological role. This sensitization is restricted to compounds that interfere with pathways associated with the targeted gene and its product.

It will be appreciated that the above cell-based assays may be performed using antisense nucleic acids complementary to any of the proliferation-required nucleic acids from Staphylococcus aureus, Salmonella typhimurium, Klebsiella pneumoniae, Pseudomonas aeruginosa, Enterococcus faecalis, Escherichia coli, Enterococcus faecalis, Haemophilus influenzae, Helicobacter pylori, or Salmonella typhi (including antisense nucleic acids complementary to SEQ ID NOs. 3796-3800, 3806-4860, 5916-10012, such as the antisense nucleic acids of SEQ ID NOs.: 8-3795) or portions thereof, antisense nucleic acids complementary to homologous coding nucleic acids or portions thereof or homologous antisense nucleic acids. In this way, the level or activity of a target, such as any of the proliferation-required polypeptides from Staphylococcus aureus, Salmonella typhimurium, Klebsiella pneumoniae, Pseudomonas aeruginosa, Enterococcus faecalis, Escherichia coli, Enterococcus faecalis, Haemophilus influenzae, Helicobacter pylori, or Salmonella typhi, (including the polypeptides of SEQ ID NOs.: 3801-3805, 4861-5915, 10013-14110), or homologous polypeptides may be reduced.

Example 11A below describes an analysis performed in Staphylococcus aureus.

### Example 11A

### Identification of the Biological Pathway in which a Gene Required for Proliferation of Staphylococcus aureus Lies

Antibiotics of various chemical classes and modes of action were purchased from chemical suppliers, for example Sigma Chemicals (St. Louis, Mo.). Stock solutions were prepared by dissolving each antibiotic in an appropriate aqueous solution based on information provided by the manufacturer. The final working solution of each antibiotic contained no more than 0.2% (w/v) of any organic solvent.

To determine its potency against a bacterial strain containing an antisense nucleic acid comprising a nucleotide sequence complementary to the nucleotide sequence encoding the Beta subunit of DNA gyrase (which is required for proliferation) under the control of a xylose inducible promoter, each antibiotic was serially diluted two- or three- fold in growth medium supplemented with the appropriate antibiotic for maintenance of the antisense construct. At least ten dilutions were prepared for each antibiotic.

Aliquots (25 μL) of each dilution were transferred to discrete wells of a 384-well microplate (the assay plate) using a multi-channel pipette. Quadruplicate wells were used for each dilution of an antibiotic under each treatment condition (plus and minus inducer). Each assay plate contained twenty wells for cell growth controls (growth medium, no antibiotic), ten wells for each treatment (plus and minus inducer, xylose, in this example). Half the assay plate contained induced cells (in this example Staphylococcus aureus cells) and appropriate concentrations of inducer (xylose, in this example) to maintain the state of induction while the other half of the assay plate contained non-induced cells maintained in the absence of inducer.

Preparation of Bacterial Cells

Cells of a bacterial clone containing a construct in which transcription of antisense comprising a nucleotide sequence complementary to the sequence encoding the Beta subunit of DNA gyrase under the control of the xylose inducible promoter (S1M10000001F08) were grown into exponential growth (OD₆₀₀ 0.2 to 0.3) and then diluted 1:100 into fresh medium containing either 12 mM or 0 mM inducer (xylose). These cultures were incubated at 37° C. for 2.5 hr. The presence of inducer (xylose) in the medium initiates and maintains production of antisense RNA from the antisense construct. After a 2.5 hr incubation, induced and non-induced cells were respectively diluted into an assay medium containing an appropriate concentration of the antibiotic for the maintenance of the antisense construct. In addition, medium used to dilute induced cells was supplemented with 24 mM xylose so that addition to the assay plate would result in a final xylose concentration of 12 mM. The cells were diluted to a final OD₆₀₀ value of 0.0004.

Induced and non-induced cell suspensions were dispensed (25 μl/well) into the appropriate wells of the assay plate as discussed previously. The plate was then loaded into a plate reader and incubated at constant temperature while cell growth was monitored in each well by the measurement of light scattering at 595 nm. Growth was monitored every 5 minutes until the cell culture attained a stationary growth phase. For each concentration of antibiotic, a percentage inhibition of growth was calculated at the time point corresponding to mid-exponential growth for the associated control wells (no antibiotic, plus or minus xylose). For each antibiotic and condition (plus or minus xylose), plots of percent inhibition versus Log of antibiotic concentration were generated and IC₅₀s determined.

A comparison of each antibiotic's IC₅₀ in the presence and absence of inducer ( xylose, in this example) reveals whether induction of the antisense construct sensitized the cell to the antibiotic's mechanism of action. If the antibiotic acts against the β subunit of DNA gyrase, the IC₅₀ of induced cells will be significantly lower than the IC₅₀ of uninduced cells.

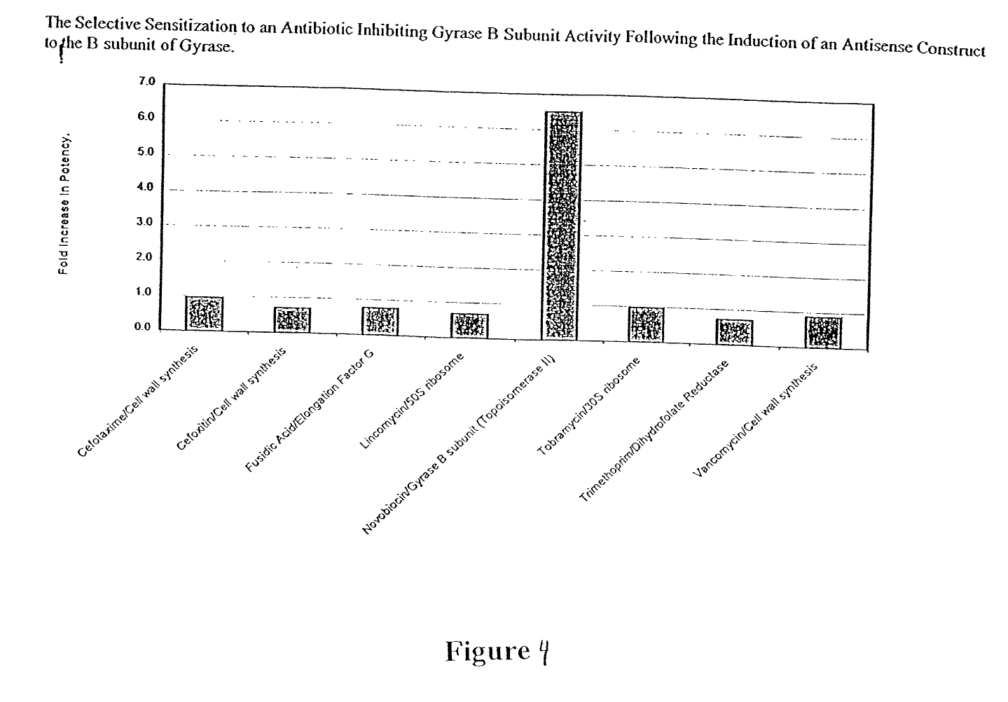

FIG. 4 lists the antibiotics tested, their targets, and their fold increase in potency between induced cells and uninduced cells. As illustrated in FIG. 4, the potency of cefotaxime, cefoxitin, fusidic acid, lincomycin, tobramycin, trimethoprim and vancomycin, each of which act on targets other than the β subunit of gyrase, was not significantly different in induced cells as compared to uninduced cells. However, the potency of novobiocin, which is known to act against the Beta subunit of DNA gyrase, was significantly different between induced cells and uninduced cells.

Thus, induction of an antisense nucleic acid comprising a nucleotide sequence complementary to the sequence encoding the β subunit of gyrase results in a selective and significant sensitization of Staphylococcus aureus cells to an antibiotic which inhibits the activity of this protein. Furthermore, the results demonstrate that induction of an antisense construct to an essential gene sensitizes a cell or microorganism to compounds that interfere with that gene product's biological role. This sensitization is apparently restricted to compounds that interfere with the targeted gene and its product.

It will be appreciated that the above cell-based assays may be performed using antisense nucleic acids complementary to any of the proliferation-required nucleic acids from Staphylococcus aureus, Salmonella typhimurium, Klebsiella pneumoniae, Pseudomonas aeruginosa, Enterococcus faecalis, Escherichia coli, Enterococcus faecalis, Haemophilus influenzae, Helicobacter pylori, or Salmonella typhi (including antisense nucleic acids complementary to SEQ ID NOs.: 3796-3800, 3806-4860, 5916-10012, such as the antisense nucleic acids of SEQ ID NOs. 8-3795), or portions thereof, antisense nucleic acids complementary to homologous coding nucleic acids or portions thereof, or homologous antisense nucleic acids. In this way, the level or activity of a target, such as any of the proliferation-required polypeptides from Staphylococcus aureus, Salmonella typhimurium, Klebsiella pneumoniae, Pseudomonas aeruginosa, Enterococcus faecalis Escherichia coli, Enterococcus faecalis, Haemophilus influenzae, Helicobacter pylori, or Salmonella typhi, or homologous polypeptides may be reduced.

Assays utilizing antisense constructs to essential genes or portions thereof can be used to identify compounds that interfere with the activity of those gene products. Such assays could be used to identify drug leads, for example antibiotics.

Panels of cells transcribing different antisense nucleic acids can be used to characterize the point of intervention of a compound affecting an essential biochemical pathway including antibiotics with no known mechanism of action.

Assays utilizing antisense constructs to essential genes can be used to identify compounds that specifically interfere with the activity of multiple targets in a pathway. Such constructs can be used to simultaneously screen a sample against multiple targets in one pathway in one reaction (Combinatorial HTS).

Furthermore, as discussed above, panels of antisense construct-containing cells may be used to characterize the point of intervention of any compound affecting an essential biological pathway including antibiotics with no known mechanism of action.

It will be appreciated that the above cell-based assays may be performed using antisense nucleic acids complementary to any of the proliferation-required nucleic acids from Staphylococcus aureus, Salmonella typhimurium, Klebsiella pneumoniae, Pseudomonas aeruginosa, Enterococcus faecalis, Escherichia coli, Enterococcus faecalis, Haemophilus influenzae, Helicobacter pylori, or Salmonella typhi (including antisense nucleic acids comprising nucleotide sequences complementary to SEQ ID NOs.: 3796-3800, 3806-4860, 5916-10012, such as the antisense nucleic acids of SEQ ID NOs. 8-3795), or portions thereof, antisense nucleic acids complementary to homologous coding nucleic acids or portions thereof, or homologous antisense nucleic acids. In this way, the level or activity of a target, such as any of the proliferation-required polypeptides from Staphylococcus aureus, Salmonella typhimurium, Klebsiella pneumoniae, Pseudomonas aeruginosa Enterococcus faecalis, Escherichia coli, Enterococcus faecalis, Haemophilus influenzae, Helicobacter pylori, or Salmonella typhi or homologous polypeptides may be reduced.

Another embodiment of the present invention is a method for determining the pathway against which a test antibiotic compound is active, in which the activity of target proteins or nucleic acids involved in proliferation-required pathways is reduced by contacting cells with a sub-lethal concentration of a known antibiotic which acts against the target protein or nucleic acid. In one embodiment, the target protein or nucleic acid corresponds to a proliferation-required nucleic acid identified using the methods described above, such as the polypeptides of SEQ ID NOs.: 3801-3805, 4861-5915, 10013-14110, or homologous polypeptides. The method is similar to those described above for determining which pathway a test antibiotic acts against, except that rather than reducing the activity or level of a proliferation-required gene product using a sub-lethal level of antisense to a proliferation-required nucleic acid, the sensitized cell is generated by reducing the activity or level of the proliferation-required gene product using a sub-lethal level of a known antibiotic which acts against the proliferation required gene product. Heightened sensitivity determines the pathway on which the test compound is active.

Interactions between drugs which affect the same biological pathway have been described in the literature. For example, Mecillinam (Amdinocillin) binds to and inactivates the penicillin binding protein 2 (PBP2, product of the mrdA in E. coli). This antibiotic interacts with other antibiotics that inhibit PBP2 as well as antibiotics that inhibit other penicillin binding proteins such as PBP3 [(Gutmann, L., Vincent, S., Billot-Klein, D., Acar, J. F., Mrena, E., and Williamson, R. (1986) Involvement of penicillin-binding protein 2 with other penicillin-binding proteins in lysis of Escherichia coli by some beta-lactam antibiotics alone and in synergistic lytic effect of amdinocillin (mecillinam). Antimicrobial Agents & Chemotherapy, 30:906-912), the disclosure of which is incorporated herein by reference in its entirety]. Interactions between drugs could, therefore, involve two drugs that inhibit the same target protein or nucleic acid or inhibit different proteins or nucleic acids in the same pathway [(Fukuoka, T., Domon, H., Kakuta, M., Ishii, C., Hirasawa, A., Utsui, Y., Ohya, S., and Yasuda, H. (1997) Combination effect between panipenem and vancomycin on highly methicillin-resistant Staphylococcus aureus. Japan. J. Antibio. 50:411-419; Smith, C. E., Foleno, B. E., Barrett, J. F., and Frosc, M. B. (1997) Assessment of the synergistic interactions of levofloxacin and ampicillin against Enterococcus faecium by the checkerboard agar dilution and time-kill methods. Diagnos. Microbiol. Infect. Disease 27:85-92; den Hollander, J. G., Horrevorts, A. M., van Goor, M. L., Verbrugh, H. A., and Mouton, J. W. (1997) Synergism between tobramycin and ceftazidime against a resistant Pseudomonas aeruginosa strain, tested in an in vitro pharmacokinetic model. Antimicrobial Agents & Chemotherapy. 41:95-110), the disclosure of all of which are incorporated herein by reference in their entireties].

Two drugs may interact even though they inhibit different targets. For example, the proton pump inhibitor, Omeprazole, and the antibiotic, Amoxycillin, two synergistic compounds acting together, can cure Helicobacter pylori infection [(Gabryelewicz, A., Laszewicz, W., Dzieniszewski, J., Ciok, J., Marlicz, K., Bielecki, D., Popiela, T., Legutko, J., Knapik, Z., Poniewierka, E. (1997) Multicenter evaluation of dual-therapy (omeprazol and amoxycillin) for Helicobacter pylori-associated duodenal and gastric ulcer (two years of the observation). J. Physiol. Pharmacol. 48 Suppl 4:93-105), the disclosure of which is incorporated herein by reference in its entirety].

The growth inhibition from the sub-lethal concentration of the known antibiotic may be at least about 5%, at least about 8%, at least about 10%, at least about 20%, at least about 30%, at least about 40%, at least about 50%, at least about 60%, or at least about 75%, or more.

Alternatively, the sub-lethal concentration of the known antibiotic may be determined by measuring the activity of the target proliferation-required gene product rather than by measuring growth inhibition.

Cells are contacted with a combination of each member of a panel of known antibiotics at a sub-lethal level and varying concentrations of the test antibiotic. As a control, the cells are contacted with varying concentrations of the test antibiotic alone. The IC₅₀ of the test antibiotic in the presence and absence of the known antibiotic is determined. If the IC₅₀s in the presence and absence of the known drug are substantially similar, then the test drug and the known drug act on different pathways. If the IC₅₀s are substantially different, then the test drug and the known drug act on the same pathway.

It will be appreciated that the above cell-based assays may be performed using a sub-lethal concentration of a known antibiotic which acts against the product of any of the proliferation-required nucleic acids from Staphylococcus aureus, Salmonella typhimurium, Klebsiella pneumoniae, Pseudomonas aeruginosa, Enterococcus faecalis, Escherichia coli, Enterococcus faecalis, Haemophilus influenzae, Helicobacter pylori, or Salmonella typhi (including the products of SEQ ID NOs: 3796-3800, 3806-4860, 5916-10012, or portions thereof, or the products of homologous coding nucleic acids or portions thereof . In this way, the level or activity of a target, such as any of the proliferation-required polypeptides from Staphylococcus aureus, Salmonella typhimurium, Klebsiella pneumoniae, Pseudomonas aeruginosa, Enterococcus faecalis, Escherichia coli, Enterococcus faecalis, Haemophilus influenzae, Helicobacter pylori, or Salmonella typhi (including the polypeptides of SEQ ID NOs.: 3801-3805, 4861-5915, 10013-14110), or homologous polypeptides may be reduced.

Another embodiment of the present invention is a method for identifying a candidate compound for use as an antibiotic in which the activity of target proteins or nucleic acids involved in proliferation-required pathways is reduced by contacting cells with a sub-lethal concentration of a known antibiotic which acts against the target protein or nucleic acid. In one embodiment, the target protein or nucleic acid is a target protein or nucleic acid corresponding to a proliferation-required nucleic acid identified using the methods described above. The method is similar to those described previously herein for identifying candidate compounds for use as antibiotics except that rather than reducing the activity or level of a proliferation-required gene product using a sub-lethal level of antisense to a proliferation-required nucleic acid, the activity or level of the proliferation-required gene product is reduced using a sub-lethal level of a known antibiotic which acts against the proliferation required gene product.

The growth inhibition from the sub-lethal concentration of the known antibiotic may be at least about 5%, at least about 8%, at least about 10%, at least about 20%, at least about 30%, at least about 40%, at least about 50%, at least about 60%, or at least about 75%, or more.

Alternatively, the sub-lethal concentration of the known antibiotic may be determined by measuring the activity of the target proliferation-required gene product rather than by measuring growth inhibition.

In order to characterize test compounds of interest, cells are contacted with a panel of known antibiotics at a sub-lethal level and one or more concentrations of the test compound. As a control, the cells are contacted with the same concentrations of the test compound alone. The IC₅₀ of the test compound in the presence and absence of the known antibiotic is determined. If the IC₅₀ of the test compound is substantially different in the presence and absence of the known drug then the test compound is a good candidate for use as an antibiotic. As discussed above, once a candidate compound is identified using the above methods its structure may be optimized using standard techniques such as combinatorial chemistry.

Representative known antibiotics which may be used in each of the above methods are provided in Table IV below. However, it will be appreciated that other antibiotics may also be used.

TABLE IV
Antibiotics and Their Targets

| | | RESISTANT |
|---|---|---|
| ANTIBIOTIC | INHIBITS/TARGET | MUTANTS |
| Inhibitors of Transcription | | |
| Rifamycin, Rifampicin | Inhibits initiation of transcription/β- | rpoB, crp, cyaA |
| Rifabutin Rifaximin | subunit RNA polymerase, rpoB | |
| Streptolydigin | Accelerates transcription chain | rpoB |
| | termination/β-subunit RNA | |
| | polymerase | |
| Streptovaricin | an acyclic ansamycin, inhibits RNA | rpoB |
| | polymerase | |
| Actinomycin D + EDTA | Intercalates between 2 successive G- | pldA |
| | C pairs, rpoB, inhibits RNA | |
| | synthesis | |
| Inhibitors of Nucleic | | |
| Acid Metabolism | | |
| Quinolones, | α subunit gyrase and/or | gyrAorB, icd, sloB |
| Nalidixic acid | topoisomerase IV, gyrA | |
| Oxolinic acid | | |
| Fluoroquinolones | α subunit gyrase, gyrA and/or | gyrA |
| Ciprofloxacin, | topoisomerase IV (probable target in | norA (efflux in |
| Norfloxacin | Staph) | Staph) |
| | | hipQ |
| Coumerins | Inhibits ATPase activity of β-subunit | |
| Novobiocin | gyrase, gyrB | gyrB, cysB, cysE, |
| | | nov, ompA |
| Coumermycin | Inhibits ATPase activity of β-subunit | gyrB, hisW |
| | gyrase, gyrB | |
| Albicidin | DNA synthesis | tsx (nucleoside |
| | | channel) |
| Metronidazole | Causes single-strand breaks in DNA | nar |
| Inhibitors of Metabolic | | |
| Pathways | | |
| Sulfonamides, | blocks synthesis of | folP, gpt, pabA, |
| Sulfanilamide | dihydrofolate,dihydro-pteroate | pabB, pabC |
| | synthesis, folP | |
| Trimethoprim, | Inhibits dihydrofolate reductase, | folA, thyA |
| | folA | |
| Showdomycin | Nucleoside analogue capable of | nupC, pnp |
| | alkylating sulfhydryl groups, inhibito | |
| | of thymidylate synthetase | |
| Thiolactomycin | type II fatty acid synthase inhibitor | emrB |
| | | fadB, emrB due to |
| | | gene dosage |
| Psicofuranine | Adenosine glycoside antibiotic, | guaA,B |
| | target is GMP synthetase | |
| Triclosan | Inhibits fatty acid synthesis | fabI (envM) |
| Diazoborines Isoniazid, | heterocyclic, contain boron, inhibit | fabI (envM) |
| Ethionamide | fatty acid synthesis, enoyl-ACP | |
| | reductase, fabI | |
| Inhibitors of Translation | | |
| Phenylpropanoids | Binds to ribosomal peptidyl transfer | |
| Chloramphenicol, | center preventing peptide | rrn, cm/A, marA, |
| | translocation/binds to S6, L3, L6, | ompF, ompR |
| | L14, L16, L25, L26, L27, but | |
| | preferentially to L16 | |
| Tetracyclines, type II | Binding to 305 ribosomal subunit, “A | clmA (cmr), mar, |
| polyketides | site | ompF |
| Minocycline | on 30S subunit, blocks peptide | |
| Doxycycline | elongation, strongest binding to S7 | |
| Macrolides (type I | Binding to 50 S ribosomal subunit, | |
| polyketides) | 23S rRNA, blocks peptide | |
| Erythromycin, | translocation, L15, L4, L12 | rrn, rplC, rplD, |
| Carbomycin, | | rplV, mac |
| Spiramycin etc | | |
| Aminoglycosides | Irreversible binding to 30S | |
| Streptomycin, | ribosomal subunit, prevents | rpsL, strC,M, ubiF |
| | translation or causes mistranslation | atpA-E, ecfB, |
| Neomycin | of mRNA/16S rRNA | hemAC,D,E,G, |
| | | topA, |
| | | rpsC,D,E, rrn, speB |
| Spectinomycin | | atpA-atpE, cpxA, |
| Kanamycin | | ecfB, hemA,B,L, |
| | | topA |
| Kasugamycin | | ksgA,B,C,D, |
| | | rplB,K, |
| Gentamicin, | | rpsI,N,M,R |
| Amikacin | | rplF, ubiF |
| Paromycin | | cpxA |
| | | rpsL |
| Lincosamides | Binding to 50 S ribosomal subunit, | |
| Lincomycin, | blocks peptide translocation | linB, rplN,O, rpsG |
| Clindamycin | | |
| Streptogramins | 2 components, Streptogramins | |
| Virginiamycin, | A&B, bind to the 50S ribosomal | |
| Pristinamycin | subunit blocking peptide | |
| Synercid: quinupristin/ | translocation and peptide bond | |
| dalfopristin | formation | |
| Fusidanes | Inhibition of elongation factor G | fusA |
| Fusidic Acid | (EF-G) prevents peptide | |
| | translocation | |
| Kirromycin (Mocimycin) | Inhibition of elongation factor TU | tufA,B |
| | (EF-Tu), prevents peptide bond | |
| | formation | |
| Pulvomycin | Binds to and inhibits EF-TU | |
| Thiopeptin | Sulfur-containing antibiotic, inhibits | rplE |
| | protein synthesis,EF-G | |
| Tiamulin | Inhibits protein synthesis | rplC, rplD |
| Negamycin | Inhibits termination process of | prfB |
| | protein synthesis | |
| Oxazolidinones Linezolid | 23S rRNA | |
| Isoniazid | | |
| | | pdx |
| Nitrofurantoin | Inhibits protein synthesis, | nfnA, B |
| | nitroreductases convert | |
| | nitrofurantoin to highly reactive | |
| | electrophilic intermediates which | |
| | attack bacterial ribosomal | |
| | proteins non-specifically | |
| Pseudomonic Acids | Inhibition of isoleucyl tRNA | ileS |
| Mupirocin (Bactroban) | synthetase-used for Staph, topical | |
| | cream, nasal spray | |
| Indolmycin | Inhibits tryptophanyl-tRNA | trpS |
| | synthetase | |
| Viomycin | | rrmA (23S rRNA |
| | | methyltransferase; |
| | | mutant has slow |
| | | growth rate, slow |
| | | chain elongation |
| | | rate, and |
| | | viomycin |
| | | resistance) |
| Thiopeptides | Binds to L11-23S RNA complex | |
| Thiostrepton | Inhibits GTP hydrolysis by EF-G | |
| | Stimulates GTP hydrolysis by EF-G | |
| Micrococcin | | |
| Inhibitors of | | |
| Cell Walls/Membranes | | |
| β-lactams | Inhibition of one or more cell wall | |
| Penicillin, Ampicillin | transpeptidases, endopeptidases, | |
| | and glycosidases (PBPs), of the 12 | ampC, ampD, |
| Methicillin, | PBPs only 2 are essential: mrdA | ampE, envZ, |
| | (PBP2) and ftsI (pbpB, PBP3) | galU, hipA, |
| | hipQ, ompC, | |
| | ompF, ompR, | |
| Cephalosporins, | | ptsI, rfa, tolD, |
| Mecillinam | Binds to and inactivates PBP2 | tolE |
| (amdinocillin) | (mrdA) | tonB |
| | Inactivates PBP3 (ftsl) | alaS, argS, crp, |
| Aztreonam | | cyaA, envB, |
| (Furazlocillin) | mrdA,B, | |
| | | mreB, C,D |
| Bacilysin, Tetaine | Dipeptide, inhib glucosamine | dppA |
| | synthase | |
| Glycopeptides Vancomycin, | Inhib G+ cell wall syn, binds to | |
| | terminal D-ala-D-ala of | |
| | pentapeptide, | |
| Polypeptides Bacitracin | Prevents dephosphorylation and | |
| | regeneration of lipid carrier | rfa |
| Cyclic lipopeptide | Disrupts multiple aspects of | |
| Daptomycin, | membrane function, including | |
| | peptidoglycan synthesis, | |
| | lipoteichoic acid synthesis, and the | |
| | bacterial membrane potential | |
| Cyclic polypeptides | Surfactant action disrupts cell | pmrA |
| Polymixin, | membrane lipids, binds lipid A | |
| | mioety of LPS | |
| Fosfomycin, | Analogue of P-enolpyruvate, | murA, crp, cyaA |
| | inhibits 1^{st} step in peptidoglycan | glpT, hipA, ptsI, |
| | synthesis - UDP-N- | uhpT |
| | acetyiglucosamine enolpyruvyl | |
| | transferase, murA. Also acts as | |
| | Immunosuppressant | |
| Cycloserine | Prevents formation of D-ala dimer, | hipA, cycA |
| | inhibits D-ala ligase, ddlA, B | |
| Alafosfalin | phosphonodipeptide, cell wall | pepA, tpp |
| | synthesis inhibitor, potentiator of | |
| | β-lactams | |
| Inhibitors of Protein | | |
| Processing/Transport | | |
| Globomycin | Inhibits signal peptidase II | lpp, dnaE |
| | (cleaves prolipoproteins | |
| | subsequent to lipid modification, | |
| | lspA | |

It will be appreciated that the above cell-based assays may be performed using a sub-lethal concentration of a known antibiotic which acts against the product of any of the proliferation-required nucleic acids from Staphylococcus aureus, Salmonella typhimurium, Klebsiella pneumoniae, Pseudomonas aeruginosa, Enterococcus faecalis, Escherichia coli Enterococcus faecalis, Haemophilus influenzae, Helicobacter pylori, or Salmonella typhi, or portions thereof, or homologous nucleic acids. In this way, the level or activity of a target, such as any of the proliferation-required polypeptides from Staphylococcus aureus, Salmonella typhimurium, Klebsiella pneumoniae, Pseudomonas aeruginosa, Enterococcus faecalis, Escherichia coli, Enterococcus faecalis, Haemophilus influenzae, Helicobacter pylori, or Salmonella typhi, or homologous polypeptides may be reduced.

### Example 12

### Transfer of Exogenous Nucleic Acid Sequences to other Bacterial Species

The ability of an antisense molecule identified in a first organism to inhibit the proliferation of a second organism (thereby confirming that a gene in the second organism which is homologous to the gene from the first organism is required for proliferation of the second organism) was validated using antisense nucleic acids which inhibit the growth of E. coli which were identified using methods similar to those described above. Expression vectors which inhibited growth of E. coli upon induction of antisense RNA expression with IPTG were transformed directly into Enterobacter cloacae, Klebsiella pneumonia or Salmonella typhimurium. The transformed cells were then assayed for growth inhibition according to the method of Example 1. After growth in liquid culture, cells were plated at various serial dilutions and a score determined by calculating the log difference in growth for INDUCED vs. UNINDUCED antisense RNA expression as determined by the maximum 10 fold dilution at which a colony was observed. The results of these experiments are listed below in Table V. If there was no effect of antisense RNA expression in a microorganism, the clone is minus in Table V. In contrast, a positive in Table V means that at least 10 fold more cells were required to observe a colony on the induced plate than on the non-induced plate under the conditions used and in that microorganism.

TABLE V
Sensitivity of Other Microorganisms to Antisense Nucleic Acids
That Inhibit Proliferation in E. coli

| Mol. No. | S. typhimurium | E. cloacae | K. pneumoniae |
|---|---|---|---|
| EcXA001 | + | + | − |
| EcXA004 | + | − | − |
| EcXA005 | + | + | + |
| EcXA006 | − | − | − |
| EcXA007 | − | + | − |
| EcXA008 | + | − | + |
| EcXA009 | − | − | − |
| EcXA010 | + | + | + |
| EcXA011 | − | + | − |
| EcXA012 | − | + | − |
| EcXA013 | + | + | + |
| EcXA014 | + | + | − |
| EcXA015 | + | + | + |
| EcXA016 | + | + | + |
| EcXA017 | + | + | + |
| EcXA018 | + | + | + |
| EcXA019 | + | + | + |
| EcXA020 | + | + | + |
| EcXA021 | + | + | + |
| EcXA023 | + | + | + |
| EcXA024 | + | − | + |
| EcXA025 | − | − | − |
| EcXA026 | + | + | − |
| EcXA027 | + | + | − |
| EcXA028 | + | − | − |
| EcXA029 | − | − | − |
| EcXA030 | + | + | + |
| EcXA031 | + | − | − |
| EcXA032 | + | + | − |
| EcXA033 | + | + | + |
| EcXA034 | + | + | + |
| EcXA035 | − | − | − |
| EcXA036 | + | − | + |
| EcXA037 | + | + | − |
| EcXA038 | + | + | + |
| EcXA039 | + | − | − |
| EcXA041 | + | + | + |
| EcXA042 | − | + | + |
| EcXA043 | − | − | − |
| EcXA044 | − | − | − |
| EcXA045 | + | + | + |
| EcXA046 | − | − | − |
| EcXA047 | + | + | − |
| EcXA048 | − | − | − |
| EcXA049 | + | − | − |
| EcXA050 | − | − | − |
| EcXA051 | + | − | − |
| EcXA052 | + | − | − |
| EcXA053 | + | + | + |
| EcXA054 | − | − | + |
| EcXA055 | + | − | − |
| EcXA056 | + | − | + |
| EcXA057 | + | + | − |
| EcXA058 | − | − | − |
| EcXA059 | + | + | + |
| EcXA060 | − | − | − |
| EcXA061 | − | − | − |
| EcXA062 | − | − | − |
| EcXA063 | + | + | − |
| EcXA064 | − | − | − |
| EcXA065 | + | + | − |
| EcXA066 | − | − | − |
| EcXA067 | − | + | − |
| EcXA068 | − | − | − |
| EcXA069 | − | + | − |
| EcXA070 | − | − | − |
| EcXA071 | + | − | − |
| EcXA072 | + | − | + |
| EcXA073 | + | + | + |
| EcXA074 | + | + | + |
| EcXA075 | + | − | − |
| EcXA076 | − | + | − |
| EcXA077 | + | + | − |
| EcXA079 | + | + | + |
| EcXA080 | + | − | − |
| EcXA082 | − | + | − |
| EcXA083 | − | − | − |
| EcXA084 | − | + | − |
| EcXA086 | − | − | − |
| EcXA087 | − | − | − |
| EcXA088 | − | − | − |
| EcXA089 | − | − | − |
| EcXA090 | − | − | − |
| EcXA091 | − | − | − |
| EcXA092 | − | − | − |
| EcXA093 | − | − | − |
| EcXA094 | + | + | + |
| EcXA095 | + | + | − |
| EcXA096 | − | − | − |
| EcXA097 | + | − | − |
| EcXA098 | + | − | − |
| EcXA099 | − | − | − |
| EcXA100 | − | − | − |
| EcXA101 | − | − | − |
| EcXA102 | − | − | − |
| EcXA103 | − | + | − |
| EcXA104 | + | + | + |
| EcXA106 | + | + | − |
| EcXA107 | − | − | − |
| EcXA108 | − | − | − |
| EcXA109 | − | − | − |
| EcXA110 | + | + | − |
| EcXA111 | − | − | − |
| EcXA112 | − | + | − |
| EcXA113 | + | + | + |
| EcXA114 | − | + | − |
| EcXA115 | − | + | − |
| EcXA116 | + | + | − |
| EcXA117 | + | − | − |
| EcXA118 | − | − | − |
| EcXA119 | + | + | − |
| EcXA120 | − | − | − |
| EcXA121 | − | − | − |
| EcXA122 | + | − | + |
| EcXA123 | + | − | − |
| EcXA124 | − | − | − |
| EcXA125 | − | − | − |
| EcXA126 | − | − | − |
| EcXA127 | + | + | − |
| EcXA128 | − | − | − |
| EcXA129 | − | + | − |
| EcXA130 | + | + | − |
| EcXA132 | − | − | − |
| EcXA133 | − | − | − |
| EcXA136 | − | − | − |
| EcXA137 | − | − | − |
| EcXA138 | + | − | − |
| EcXA139 | − | − | − |
| EcXA140 | + | − | − |
| EcXA141 | + | − | − |
| EcXA142 | − | − | − |
| EcXA143 | − | + | − |
| EcXA144 | + | + | − |
| EcXA145 | − | − | − |
| EcXA146 | − | − | − |
| EcXA147 | − | − | − |
| EcXA148 | − | − | − |
| EcXA149 | + | + | + |
| EcXA150 | − | − | − |
| EcXA151 | + | − | − |
| EcXA152 | − | − | − |
| EcXA153 | + | + | − |
| ExXA154 | − | − | − |
| EcXA155 | − | − | ND |
| EcXA156 | − | + | − |
| EcXA157 | − | − | − |
| EcXA158 | − | − | − |
| EcXA159 | + | − | − |
| EcXA160 | + | − | − |
| EcXA162 | − | − | − |
| EcXA163 | − | − | − |
| EcXA164 | − | − | − |
| EcXA165 | − | − | − |
| EcXA166 | − | − | − |
| EcXA167 | − | − | − |
| EcXA168 | − | − | − |
| EcXA169 | − | + | − |
| EcXA171 | − | − | − |
| EcXA172 | − | − | − |
| EcXA173 | − | − | − |
| EcXA174 | − | − | − |
| EcXA175 | − | − | − |
| EcXA176 | − | − | − |
| EcXA178 | − | − | − |
| EcXA179 | − | − | − |
| EcXA180 | + | − | − |
| EcXA181 | − | − | − |
| EcXA182 | − | − | − |
| EcXA183 | − | − | − |
| EcXA184 | − | − | − |
| EcXA185 | − | − | − |
| EcXA186 | − | − | − |
| EcXA187 | + | + | + |
| EcXA189 | + | − | − |
| EcXA190 | + | + | + |
| EcXA191 | + | + | − |
| EcXA192 | − | + | − |

Thus, the ability of an antisense nucleic acid which inhibits the proliferation of Staphylococcus aureus, Salmonella typhimurium, Klebsiella pneumoniae, Pseudomonas aeruginosa, Enterococcus faecalis, Escherichia coli, Enterococcus faecalis, Haemophilus influenzae, Helicobacter pylori, or Salmonella typhi to inhibit the growth of other organims may be evaluated by transforming the antisense nucleic acid directly into species other than the organism from which they were obtained. In particular, the ability of the antisense nucleic acid to inhibit the growth of Anaplasma marginale, Aspergillus fumigatus, Bacillus anthracis, Bacterioides fragilis Bordetella pertussis, Burkholderia cepacia, Campylobacter jejuni, Candida albicans, Candida glabrata (also called Torulopsis glabrata), Candida tropicalis, Candida parapsilosis, Candida guilliermondii, Candida krusei, Candida kefyr (also called Candida pseudotropicalis), Candida dubliniensis, Chlamydia pneumoniae, Chlamydia trachomatus, Clostridium botulinum, Clostridium difficile, Clostridium perfringens, Coccidiodes immitis, Corynebacterium diptheriae, Cryptococcus neoformans, Enterobacter cloacae, Enterococcus faecalis, Enterococcus faecium, Escherichia coli, Haemophilus influenzae, Helicobacter pylori, Histoplasma capsulatum, Klebsiella pneumoniae, Listeria monocytogenes, Mycobacterium leprae, Mycobacterium tuberculosis, Neisseria gonorrhoeae, Neisseria meningitidis, Nocardia asteroides, Pasteurella haemolytica, Pasteurella multocida, Pneumocystis carinii, Proteus vulgaris, Pseudomonas aeruginosa, Salmonella bongori, Salmonella cholerasuis, Salmonella enterica, Salmonella paratyphi, Salmonella typhi, Salmonella typhimurium, Staphylococcus aureus, Listeria monocytogenes, Moxarella catarrhalis, Shigella boydii, Shigella dysenteriae, Shigella flexneri, Shigella sonnei, Staphylococcus epidermidis, Streptococcus pneumoniae, Streptococcus mutans, Treponema pallidum, Yersinia enterocolitica, Yersinia pestis or any species falling within the genera of any of the above species. may be evaluated. In some embodiments of the present invention, the ability of the antisense nucleic acid to inhibit the growth of an organism other than E. coli may be evaluated. In such embodiments, the antisense nucleic acids are inserted into expression vectors functional in the organisms in which the antisense nucleic acids are evaluated.

It will be appreciated that the above methods for evaluating the ability of an antisense nucleic acid to inhibit the proliferation of a heterologous organism may be performed using antisense nucleic acids complementary to any of the proliferation-required nucleic acids from Staphylococcus aureus, Salmonella typhimurium, Klebsiella pneumoniae, Pseudomonas aeruginosa, Enterococcus faecalis, Escherichia coli, Enterococcus faecalis, Haemophilus influenzae, Helicobacter pylori, or Salmonella typhi (including antisense nucleic acids complementary to SEQ ID NOs.: 3796-3800, 3806-4860, 5916-10012, such as the antisense nucleic acids of SEQ ID NOs.: 8-3795) or portions thereof, antisense nucleic acids complementary to homologous coding nucleic acids or portions thereof, or homologous antisense nucleic acids.

Those skilled in the art will appreciate that a negative result in a heterologous cell or microorganism does not mean that that cell or microorganism is missing that gene nor does it mean that the gene is unessential. However, a positive result means that the heterologous cell or microorganism contains a homologous gene which is required for proliferation of that cell or microorganism. The homologous gene may be obtained using the methods described herein. Those cells that are inhibited by antisense may be used in cell-based assays as described herein for the identification and characterization of compounds in order to develop antibiotics effective in these cells or microorganisms. Those skilled in the art will appreciate that an antisense molecule which works in the microorganism from which it was obtained will not always work in a heterologous cell or microorganism.

### Example 12A

### Transfer of Exogenous Nucleic Acid Sequences to other Bacterial Species Using the Staphylococcus aureus, Salmonella typhimurium, Klebsiella pneumoniae, Pseudomonas aeruginosa, Enterococcus faecalis, Escherichia coli, Enterococcus faecalis, Haemophilus influenzae, Helicobacter pylori, or Salmonella typhi Expression Vectors or Expression Vectors Functional in Bacterial Species other than Staphylococcus aureus, Salmonella typhimurium, Klebsiella pneumoniae, Pseudomonas aeruginosa, Enterococcus faecalis, Escherichia coli, Enterococcus faecalis, Haemophilus influenzae, Helicobacter pylori, or Salmonella typhi.

The antisense nucleic acids that inhibit the growth of Staphylococcus aureus, Salmonella typhimurium, Klebsiella pneumoniae, Pseudomonas aeruginosa, Enterococcus faecalis, Escherichia coli, Enterococcus faecalis, Haemophilus influenzae, Helicobacter pylori, or Salmonella typhi, or portions thereof, may also be evaluated for their ability to inhibit the growth of cells or microorganisms other than Staphylococcus aureus, Salmonella typhimurium, Klebsiella pneumoniae, Pseudomonas aeruginosa, Enterococcus faecalis, Escherichia coli, Enterococcus faecalis, Haemophilus influenzae, Helicobacter pylori, or Salmonella typhi. For example, the antisense nucleic acids that inhibit the growth of Staphylococcus aureus, Salmonella typhimurium, Klebsiella pneumoniae, Pseudomonas aeruginosa, Enterococcus faecalis, Escherichia coli, Enterococcus faecalis, Haemophilus influenzae, Helicobacter pylori, or Salmonella typhi may be evaluated for their ability to inhibit the growth of other organisms. In particular, the ability of the antisense nucleic acid to inhibit the growth of Anaplasma marginale, Aspergillus fumigatus, Bacillus anthracis, Bacterioides fragilis Bordetella pertussis, Burkholderia cepacia, Campylobacter jejuni, Candida albicans, Candida glabrata (also called Torulopsis glabrata), Candida tropicalis, Candida parapsilosis, Candida guilliermondii, Candida krusei, Candida kefyr (also called Candida pseudotropicalis), Candida dubliniensis, Chlamydia pneumoniae, Chlamydia trachomatus, Clostridium botulinum, Clostridium difficile, Clostridium perfringens, Coccidiodes immitis, Corynebacterium diptheriae, Cryptococcus neoformans, Enterobacter cloacae, Enterococcus faecalis, Enterococcus faecium, Escherichia coli, Haemophilus influenzae, Helicobacter pylori, Histoplasma capsulatum, Klebsiella pneumoniae, Listeria monocytogenes, Mycobacterium leprae, Mycobacterium tuberculosis, Neisseria gonorrhoeae, Neisseria meningitidis, Nocardia asteroides, Pasteurella haemolytica, Pasteurella multocida, Pneumocystis carinii Proteus vulgaris, Pseudomonas aeruginosa, Salmonella bongori, Salmonella cholerasuis, Salmonella enterica, Salmonella paratyphi, Salmonella typhi, Salmonella typhimurium, Staphylococcus aureus, Listeria monocytogenes, Moxarella catarrhalis, Shigella boydii, Shigella dysenteriae, Shigella flexneri, Shigella sonnei, Staphylococcus epidermidis, Streptococcus pneumoniae, Streptococcus mutans, Treponema pallidum, Yersinia enterocolitica, Yersinia pestis or any species falling within the genera of any of the above species may be evaluated. In some embodiments of the present invention, the ability of the antisense nucleic acid to inhibit the growth of an organism other than E. coli may be evaluated.

In such methods, expression vectors in which the expression of an antisense nucleic acid that inhibits the growth of Staphylococcus aureus, Salmonella typhimurium, Klebsiella pneumoniae, Pseudomonas aeruginosa, Enterococcus faecalis, Escherichia coli, Enterococcus faecalis, Haemophilus influenzae, Helicobacter pylori, or Salmonella typhi is under the control of an inducible promoter are introduced into the cells or microorganisms in which they are to be evaluated. In some embodiments, the antisense nucleic acids may be evaluated in cells or microorganisms which are closely related to Staphylococcus aureus, Salmonella typhimurium, Klebsiella pneumoniae, Pseudomonas aeruginosa, Enterococcus faecalis, Escherichia coli, Enterococcus faecalis, Haemophilus influenzae, Helicobacter pylori, or Salmonella typhi. The ability of these antisense nucleic acids to inhibit the growth of the related cells or microorganisms in the presence of the inducer is then measured.

For example, thirty-nine antisense nucleic acids which inhibited the growth of Staphylococcus aureus were identified using methods such as those described herein and were inserted into an expression vector such that their expression was under the control of a xylose-inducible Xyl-T5 promoter. A vector with Green Fluorescent Protein (GFP) under control of the Xyl-T5 promoter was used to show that expression from the Xyl-T5 promoter in Staphylococcus epidermidis was comparable to that in Staphylococcus aureus.

The vectors were introduced into Staphylococcus epidermidis by electroporation as follows: Staphylococcus epidermidis was grown in liquid culture to mid-log phase and then harvested by centrifugation. The cell pellet was resuspended in 1/3 culture volume of ice-cold EP buffer (0.625 M sucrose, 1 mM MgCl₂, pH=4.0), and then harvested again by centrifugation. The cell pellet was then resuspended with 1/40 volume EP buffer and allowed to incubate on ice for 1 hour. The cells were then frozen for storage at −80° C. For electroporation, 50 μl of thawed electrocompetent cells were combined with 0.5 μg plasmid DNA and then subjected to an electrical pulse of 10 kV/cm, 25 uFarads, 200 ohm using a biorad gene pulser electroporation device. The cells were immediately resuspended with 200 μl outgrowth medium and incubated for 2 hours prior to plating on solid growth medium with drug selection to maintain the plasmid vector. Colonies resulting from overnight growth of these platings were selected, cultured in liquid medium with drug selection, and then subjected to dilution plating analysis as described for Staphylococcus aureus in Example 10 above to test growth sensitivity in the presence of the inducer xylose.

The results are shown in Table VI below. The first column indicates the Molecule Number of the Staphylococcus aureus antisense nucleic acid which was introduced into Staphylococcus epidermidis. The second column indicates whether the antisense nucleic acid inhibited the growth of Staphylococcus epidermidis, with a indicating that growth was inhibited. Of the 39 Staphylococcus aureus antisense nucleic acids evaluated, 20 inhibited the growth of Staphylococcus epidermidis.

TABLE VI
Sensitivity of Other Microorganisms to Antisense Nucleic Acids
That Inhibit Proliferation of Staphylococcus aureus

| Mol. No. | S. epidermidis |
|---|---|
| SaXA005 | + |
| SaXA007 | + |
| SaXA008 | + |
| SaXA009 | + |
| SaXA010 | − |
| SaXA011 | − |
| SaXA012 | − |
| SaXA013 | − |
| SaXA015 | + |
| SaXA017 | − |
| SaXA022 | + |
| SaXA023 | − |
| SaXA024 | − |
| SaXA025 | + |
| SaXA026 | + |
| SaXA027 | − |
| SaXA027b | − |
| SaXA02c | − |
| SaXA028 | − |
| SaXA029 | + |
| SaXA030 | − |
| SaXA032 | + |
| SaXA033 | + |
| SaXA034 | − |
| SaXA035 | + |
| SaXA037 | − |
| SaXA039 | − |
| SaXA042 | − |
| SaXA043 | − |
| SaXA044 | − |
| SaXA045 | + |
| SaXA051 | + |
| SaXA053 | − |
| SaXA056b | − |
| SaXA059a | + |
| SaXA060 | − |
| SaXA061 | + |
| SaXA062 | + |
| SaXA063 | − |
| SaXA065 | − |

Although the results shown above were obtained using a subset of the nucleic acids of the present invention, it will be appreciated that similar analyses may be performed using the other nucleic acids of the present invention to determine whether they inhibit the proliferation of cells or microorganisms other than Staphylococcus aureus, Salmonella typhimurium, Klebsiella pneumoniae, Pseudomonas aeruginosa, Enterococcus faecalis, Escherichia coli, Enterococcus faecalis, Haemophilus influenzae, Helicobacter pylori, or Salmonella typhi.

Thus, it will be appreciated that the above methods for evaluating the ability of an antisense nucleic acid to inhibit the proliferation of a heterologous organism may be performed using antisense nucleic acids complementary to any of the proliferation-required nucleic acids from Staphylococcus aureus, Salmonella typhimurium, Klebsiella pneumoniae, Pseudomonas aeruginosa, Enterococcus faecalis, Escherichia coli, Enterococcus faecalis, Haemophilus influenzae, Helicobacter pylori, or Salmonella typhi, (including antisense nucleic acids complementary to SEQ ID NOs.: 3796-3800, 3806-4860, 5916-10012, such as the antisense nucleic acids of SEQ ID NOs.: 8-3795) or portions thereof, antisense nucleic acids complementary to homologous coding nucleic acids or portions thereof, or homologous antisense nucleic acids.

### Example 12C

As a demonstration of the methodology required to find homologues to an essential gene, nine prokaryotic organisms were analyzed and compared in detail. First, the most reliable source of gene sequences for each organism was assessed by conducting a survey of the public and private data sources. The nine organisms studied are Escherichia coli, Haemophilus influenzae, Helicobacter pylori, Klebsiella pneumoniae, Pseudomonas aeruginosa, Staphylococcus aureus, Streptococcus pneumoniae and Salmonella typhi. Full-length gene protein and nucleotide sequences for these organisms were assembled from various sources. For Escherichia coli, Haemophilus influenzae and Helicobacter pylori, gene sequences were adopted from the public sequencing projects, and derived from the GenPept 115 database (available from NCBI). For Pseudomonas aeruginosa, gene sequences were adopted from the Pseudomonas genome sequencing project (downloaded from http://www.pseudomonas.com). For Klebsiella pneumoniae, Staphylococcus aureus, Streptococcus pneumoniae and Salmonella typhi, genomic sequences from PathoSeq v 4.1 (Mar 2000 release) was reanalyzed for ORFs using the gene finding software GeneMark v 2.4a, which was purchased from GenePro Inc. 451 Bishop St., N.W., Suite B, Atlanta, Ga., 30318, USA.

Subsequently, the essential genes found by the antisense methodology were compared to the derived proteomes of interest, in order to find all the homologous genes to a given gene. This comparison was done using the FASTA program v3.3. Genes were considered homologues if they were greater than 25% identical and the alignment between the two genes covered more than 70% of the length of one of the genes. The best homologue for each of the nine organisms, defined as the most significantly scoring match which also fulfilled the above criteria, was reported in Table VIIA. Table VIIA lists the best ORF identified as described above (column labelled LOCUSID), the SEQ ID, % identity, and the amount of the protein which aligns well with the query sequence (coverage) for the gene identified in each of the nine organisms evaluated as described above.

Table VIIB lists the PathoSeq cluster ID for genes identified as being required for proliferation in Enterococcus faecalis, Escherichia coli, Pseudomonas aeruginosa, and Staphylococcus aureus using the methods described herein. As indicated in the column labelled PathoSeq cluster ID, these sequences share homology to one another and were consequently grouped within the same PathoSeq cluster. Thus, the methods described herein identified genes required for proliferation in several species which share homology.

TABLE VIIA

| | | Escherichia | Enterococcus | Haemophilus | Helicobacter | Klebsiella | Pseudomonas | Staphylococcus | Streptococcus | Salmonella | | | |
|---|---|---|---|---|---|---|---|---|---|---|---|---|---|
| LOCUSID | Data | coli | faecalis | influenzae | pylori | pneumoniae | aeruginosa | aureus | pneumoniae | typhi | | | |
| EFA100001 | SeqID | 10430 | 10618 | 10998 | 11603 | 11739 | | 12309 | 13524 | 14040 | | | |
| | IDENTITY | 27% | 100% | 28% 28% | 29% | | 52% | 55% | 28% | | | | |
| | COVERAGE | 99% | 100% | 101% | 79% | 77% | | 98% | 98% | 98% | | | |
| EFA100023 | SeqID | | 10505 | | | | | 12860 | 13392 | | | | |
| | IDENTITY | | 100% | | | | | 27% | 39% | | | | |
| | COVERAGE | | 100% | | | | | 95% | 101% | | | | |
| EFA100065 | SeqID | 10322 | 10813 | 11177 | 11351 | | 12018 | 12820 | 13186 | 13733 | | | |
| | IDENTITY | 49% | 100% | 49% | 44% | | 48% | 59% | 65% | 48% | | | |
| | COVERAGE | 96% | 100% | 95% | 96% | | 97% | 97% 98% | 96% | | | | |
| EFA100151 | SeqID | 10128 | 10516 | 11247 | 11340 | | 11891 | 12529 | 13362 | | | | |
| | IDENTITY | 50% | 100% | 37% | 46% | | 49% | 54% | 51% | | | | |
| | COVERAGE | 99% | 100% | 100% | 100% | | 100% | 99% | 100% | | | | |
| EFA100157 | SeqID | | 10673 | | 11448 | 12352 | 13176 | | | | | | |
| | IDENTITY | | 100% | | 39% | 64% | 74% | | | | | | |
| | COVERAGE | | 100% | | 98% | 98% | 99% | | | | | | |
| EFA100165 | SeqID | 10031 | 10637 | 11189 | 11564 | | 12009 | 12614 | 13399 | 14078 | | | |
| | IDENTITY | 31% | 100% | 33% | 28% | | 32% | 29% | 27% | 29% | | | |
| | COVERAGE | 97% | 100% | 98% | 100% | | 96% | 90% | 96% | 97% | | | |
| EFA100190 | SeqID | 10364 | 10480 | 11061 | 11408 | 11659 | 11996 | 12444 | 13232 | 13966 | | | |
| | IDENTITY | 54% | 100% | 57% | 55% | 55% | 54% | 78% | 80% | 54% | | | |
| | COVERAGE | 100% | 101% | 100% | 99% | 90% | 100% | 101% | 101% | 101% | | | |
| EFA100194 | SeqID | 10336 | 10540 | 11120 | 11426 | | 11989 | 12230 | 13222 | 14096 | | | |
| | IDENTITY | 60% | 100% | 62% | 62% | | 60% | 85% | 86% | 61% | | | |
| | COVERAGE | 100% | 101% | 100% | 102% | 100% | 101% | 92% | 101% | | | | |
| EFA100200 | SeqID | 10323 | 10798 | 11193 | | | 12020 | 12527 | 13561 | 13731 | | | |
| | IDENTITY | 39% | 100% | 38% | | | 40% | 50% | 59% | 39% | | | |
| | COVERAGE | 85% | 100% | 87% | | | 85% | 85% | 88% | 85% | | | |
| EFA100210 | SeqID | 10352 | 10560 | 11104 | 11439 | | 5171 | 12260 | 13204 | 13968 | | | |
| | IDENTITY | 53% | 100% | 53% | 53% | | 54% | 74% | 93% | 53% | | | |
| | COVERAGE | 95% | 101% | 95% | 94% | | 95% | 101% | 94% | 95% | | | |
| EFA100211 | SeqID | 10351 | 10523 | 11105 | 11438 | | 11992 | 12214 | 13205 | | | | |
| | IDENTITY | 46% | 100% | 46% | 39% | | 43% | 69% | 63% | | | | |
| | COVERAGE | 87% | 101% | 87% | 81% | | 87% | 97% | 81% | _______ | | | |
| EFA100289 | SeqID | 10284 | 10810 | | | | 11827 | | 13245 | | | | |
| | IDENTITY | 30% | 100% | | | | 31% | | 25% | | | | |
| | COVERAGE | 85% | 100% | | | | 90% | | 84% | | | | |
| EFA100295 | SeqID | 10045 | 10517 | 11174 | 11601 | | 11937 | 12390 | 13616 | 13911 | | | |
| | IDENTITY | 43% | 100% | 41% | 41% | | 45% | 44% | 45% | 43% | | | |
| | COVERAGE | 92% | 101% | 95% | 97% | | 97% | 99% | 94% | 72% | | | |
| EFA100312 | SeqID | | 10641 | | | | | 12178 | | | | | |
| | IDENTITY | | 100% | | | | | 33% | | | | | |
| | COVERAGE | | 100% | | | | | 88% | | | | | |
| EFA100329 | SeqID | | 10782 | | | | | | | | | | |
| | IDENTITY | | 100% | | | | | | | | | | |
| | COVERAGE | | 100% | | | | | | | | | | |
| EFA100394 | SeqID | 10465 | 10675 | 11238 | 11563 | | 11961 | 13003 | 13684 | 13853 | | | |
| | IDENTITY | 43% | 100% | 43% | 42% | | 44% | 66% | 72% | 44% | | | |
| | COVERAGE | 108% | 100% | 109% | 101% | | 108% | 99% | 100% | 108% | | | |
| EFA100397 | SeqID | 10027 | 10773 | 11185 | | | 12012 | 12396 | 13478 | 14074 | | | |
| | IDENTITY | 31% | 100% | 29% | | | 29% | 43% | 46% | 31% | | | |
| | COVERAGE | 96% | 100% | 98% | | | 93% | 91% | 97% | 93% | | | |
| EFA100399 | SeqID | 10295 | 10766 | 11196 | 11483 | | 11791 | 12281 | 13413 | 13739 | | | |
| | IDENTITY | 63% | 100% | 59% | 59% | | 58% | 72% | 76% | 63% | | | |
| | COVERAGE | 98% | 100% | 98% | 99% | | 101% | 99% | 100% | 98% | | | |
| EFA100426 | SeqID | 10224 | 10702 | | | 11638 | | 12139 | 13348 | 13957 | | | |
| | IDENTITY | 28% | 100% | | | 29% | | 42% | 41% | 28% | | | |
| | COVERAGE | 99% | 101% | 99% | | 91% | 109% | 99% | | | | | |
| EFA100478 | SeqID | 10486 | 11135 | 11338 | | | 12986 | 13184 | | | | | |
| | IDENTITY | 100% | 29% | 31% | | | 44% | 43% | | | | | |
| | COVERAGE | 100% | 72% | 70% | | | 99% | 98% | | | | | |
| EFA100615 | SeqID | 10501 | 11139 | | | 12028 | 12641 | 13331 | | | | | |
| | IDENTITY | 100% | 44% | | | 47% | 61% | 78% | | | | | |
| | COVERAGE | 100% | 82% | | | 81% | 100% | 100% | | | | | |
| EFA100617 | SeqID | 10314 | 10764 | 11216 | 11391 | | 5198 | 12322 | 13381 | 13765 | | | |
| | IDENTITY | 43% | 100% | 43% | 44% | | 51% | 63% | 69% | 44% | | | |
| | COVERAGE | 95% | 100% | 96% | 78% | | 73% | 84% | 82% | 93% | | | |
| EFA100641 | SeqID | 10205 | 10793 | | | | 11896 | 12862 | 13334 | | | | |
| | IDENTITY | 28% | 100% | | | | 31% | 50% | 32% | | | | |
| | COVERAGE | 79% | 100% | | | | 74% | 85% | 82% | | | | |
| EFA100642 | SeqID | | 10792 | 11520 | 12023 | 12493 | 13367 | | | | | | |
| | IDENTITY | | 100% | 46% | 46% | 73% | 69% | | | | | | |
| | COVERAGE | | 100% | 100% | 101% | 100% | 100% | | | | | | |
| EFA100668 | SeqID | 10026 | 10679 | 11184 | 11613 | | 12013 | 12891 | 13505 | 14073 | | | |
| | IDENTITY | 28% | 100% | 28% | 29% | | 28% | 29% | 50% | 27% | | | |
| | COVERAGE | 83% | 100% | 76% | 78% | | 92% | 82% | 99% | 95% | | | |
| EFA100689 | SeqID | | 10717 | | | | | 12523 | 13698 | | | | |
| | IDENTITY | | 100% | | 33% | 33% | | | | | | | |
| | COVERAGE | | 100% | | | | | 100% | 100% | | | | |
| EFA100704 | SeqID | 10362 | 10482 | 11059 | 11415 | | 11995 | 12442 | 13171 | 13964 | | | |
| | IDENTITY 78% | 100% | 78% | 77% | | 75% | 90% | 78% | 77% | | | | |
| | COVERAGE | 100% | 100% | 100% | 101% | | 101% | 100% | 101% | 100% | | | |
| EFA100739 | SeqID | 10111 | 10537 | 11052 | 11429 | 11651 | 11876 | 12228 | 13220 | 14010 | | | |
| | IDENTITY 71% | 100% | 69% | 63% | 70% | 71% | 84% | 84% | 70% | | | | |
| | COVERAGE | 83% | 101% | 83% | 86% | 87% | 83% | 87% | 87% | 87% | | | |
| EFA100740 | SeqID | 10075 | 10536 | 11008 | 11348 | 11633 | 11942 | 12227 | 13219 | 13717 | | | |
| | IDENTITY 45% | 100% | 47% | 30% | 45% | 48% | 64% | 60% | 44% | | | | |
| | COVERAGE | 94% | 100% | 94% | 93% | 94% | 82% | 94% | 93% | 94% | | | |
| EFA100741 | SeqID | 10339 | 10535 | 11118 | 11430 | | 11991 | 12226 | 13218 | 14098 | | | |
| | IDENTITY 40% | 100% | 37% | 34% | | 39% | 48% | 60% | 40% | | | | |
| | COVERAGE | 103% | 100% | | 102% | 101% | 102% | 101% | 100% | 103% | | | |
| EFA100742 | SeqID | 10340 | 10534 | 11116 | 11431 | | 5160 | 12225 | 13217 | 14099 | | | |
| | IDENTITY 52% | 100% | 52% | 39% | | 46% | 79% | 88% | 52% | | | | |
| | COVERAGE | 99% | 101% | 99% | 92% | | 99% | 101% | 101% | 99% | | | |
| EFA100748 | SeqID | 10287 | 10483 | 11004 | 11523 | 11690 | 11944 | 12595 | | 13868 | | | |
| | IDENTITY 41% | 100% | 39% | 29% | 42% | 44% | 52% | | 41% | | | | |
| | COVERAGE | 99% | 100% | 99% | 94% | 98% | 100% | 100% | | 100% | | | |
| EFA100756 | SeqID | 10112 | 10575 | | 11396 | | 11875 | 12327 | 13343 | 14009 | | | |
| | IDENTITY 49% | 100% | 43% | | 45% | 64% | 62% | 47% | | | | | |
| | COVERAGE | 75% | 102% | | 75% | | 81% | 94% | 94% | 75% | | | |
| EFA100757 | SeqID | 10155 | 10897 | | | | | | | | | | |
| | IDENTITY | 27% | 100% | | | | | | | | | | |
| | COVERAGE | 85% | 100% | | | | | | | | | | |
| EFA100783 | SeqID | 10035 | 10811 | 10986 | | 11543 | 11953 | 12738 | 13261 | 13914 | | | |
| | IDENTITY | 32% | 100% | 34% | 86% | | 37% | 77% | 75% | 31% | | | |
| | COVERAGE | 104% | 100% | 83% | 100% | | 78% | 100% | 99% | 99% | | | |
| EFA100795 | SeqID | 10863 | | | | | | 13416 | | | | | |
| | IDENTITY | 100% | | | | | | 50% | | | | | |
| | COVERAGE | 101% | | | | | | 101% | | | | | |
| EFA100798 | SeqID | 10382 | 10818 | 11153 | 11550 | | 11775 | | 13641 | | | | |
| | IDENTITY | 62% | 100% | 61% | 56% | | 63% | | 85% | | | | |
| | COVERAGE | 95% | 100% | 95% | 89% | | 92% | | 96% | | | | |
| EFA100811 | SeqID | | 10546 | | | | | 12236 | 13439 | | | | |
| | IDENTITY | | 100% | | | | | 48% | 58% | | | | |
| | COVERAGE | | 101% | | | | | 98% | 99% | | | | |
| EFA100870 | SeqID | 10439 | 10627 | 11036 | 11410 | | 5179 | 12446 | 13646 | 14042 | | | |
| | IDENTITY | 47% | 100% | 46% | 52% | | 46% | 72% | 78% | 46% | | | |
| | COVERAGE | 114% | 100% | 117% | 79% | | 116% | 99% | 98% | 114% | | | |
| EFA100914 | SeqID | 10399 | 10579 | 11018 | 11617 | 11758 | 12111 | 12368 | 13230 | 14065 | | | |
| | IDENTITY | 40% | 100% | 40% | 34% | 40% | 40% | 59% | 63% | 40% | | | |
| | COVERAGE | 102% | 100% | 102% | 101% | 102% | 102% | 101% | 95% | 102% | | | |
| EFA100919 | SeqID | 10269 | 10491 | 11127 | 11419 | | 11809 | 12556 | 13594 | 13874 | | | |
| | IDENTITY | 44% | 100% | 45% | 40% | | 46% | 55% | 63% | 45% | | | |
| | COVERAGE | 101% | 100% | 101% | 99% | | 101% | 101% | 100% | 101% | | | |
| EFA100955 | SeqID | 10333 | 10542 | 11123 | 11582 | 11627 | 5158 | 12232 | 13224 | 14093 | | | |
| | IDENTITY | 48% | 100% | 48% | 42% | 49% | 43% | 65% | 76% | 48% | | | |
| | COVERAGE | 98% | 101% | 98% | 98% | 79% | 98% | 99% | 101% | 98% | | | |
| EFA100970 | SeqID | | 10906 | | | | | | | | | | |
| | IDENTITY | | 100% | | | | | | | | | | |
| | COVERAGE | | 100% | | | | | | | | | | |
| EFA100978 | SeqID | 10334 | 10541 | 11122 | 11583 | | 11987 | 12231 | 13223 | 14094 | | | |
| | IDENTITY | 46% | 100% | 46% | 35% | | 45% | 71% | 70% | 46% | | | |
| | COVERAGE | 100% | 100% | 99% | 98% | | 102% | 101% | 100% | 100% | | | |
| EFA100991 | SeqID | 10221 | 10681 | 11210 | 11607 | 11668 | 11801 | 12289 | 13191 | 14027 | | | |
| | IDENTITY | 42% | 100% | 40% | 29% | 42% | 39% | 49% | 56% | 30% | | | |
| | COVERAGE | 91% | 100% | 93% | 98% | 94% | 91% | 93% | 92% | 93% | | | |
| EFA101022 | SeqID | 10260 | 10875 | 10982 | 11401 | | 11945 | 12715 | 13251 | 14086 | | | |
| | IDENTITY | 59% | 100% | 58% | 50% | | 61% | 76% | 86% | 56% | | | |
| | COVERAGE | 85% | 101% | 85% | 88% | | 85% | 85% | 89% | 89% | | | |
| EFA101060 | SeqID | | 10722 | | 11575 | 11646 | 11957 | 12504 | 13554 | | | | |
| | IDENTITY | | 100% | | 35% | 37% | 34% | 71% | 67% | | | | |
| | COVERAGE | | 101% | 83% | 77% | 97% | 100% | 101% | | | | | |
| EFA101086 | SeqID | 10315 | 10763 | 11215 | 11454 | 11716 | 12052 | 12953 | 13662 | 13764 | | | |
| | IDENTITY | 37% | 100% | 37% | 27% | 38% | 35% | 57% | 55% | 36% | | | |
| | COVERAGE | 91% | 100% | 89% | 98% | 91% | 92% | 98% | 95% | 93% | | | |
| EFA101120 | SeqID | 10017 | 10687 | 11219 | 11331 | | 12057 | 12505 | 13498 | 14012 | | | |
| | IDENTITY | 30% | 100% | 31% | 27% | | 29% | 26% | 64% | 29% | | | |
| | COVERAGE | 102% | 100% | 102% | 74% | | 103% | 99% | 98% | 103% | | | |
| EFA101121 | SeqID | | 10686 | | | | | 12606 | 13600 | | | | |
| | IDENTITY | | 100% | | 38% | 50% | | | | | | | |
| | COVERAGE | | 100% | | 98% | 99% | | | | | | | |
| EFA101123 | SeqID | 10420 | 10748 | 11131 | 11478 | 11629 | 11820 | 12674 | 13265 | 13783 | | | |
| | IDENTITY | 43% | 100% | 39% | 33% | 43% | 40% | 70% | 70% | 42% | | | |
| | COVERAGE | 98% | 100% | 97% | 97% | 94% | 96% | 99% | 100% | 98% | | | |
| EFA101141 | SeqID | 10436 | 10614 | 11071 | 11573 | | 5181 | 12450 | 13246 | 14045 | | | |
| | IDENTITY | 35% | 100% | 40% | 35% | | 40% | 60% | 70% | 31% | | | |
| | COVERAGE | 94% | 101% | 96% | 95% | 95% | | 98% | 101% | 96% | | | |
| EFA101150 | eqID | 10174 | 10719 | 11221 | 11556 | | 11880 | 12985 | 13385 | 13943 | | | |
| | IDENTITY | 35% | 100% | 36% | 26% | | 33% | 45% | 58% | 36% | | | |
| | COVERAGE | 100% | 100% | 100% | 102% | 100% | 100% | 100% | 73% | | | | |
| EFA101159 | SeqID | 10359 | 10543 | 11097 | 11442 | | 5176 | 12235 | 13197 | 13974 | | | |
| | IDENTITY | 55% | 100% | 52% | 48% | 49% | 58% | 89% | 53% | | | | |
| | COVERAGE | 100% | 101% | 100% | 81% | | 101% | 99% | 99% | 100% | | | |
| EFA101160 | SeqID | 10358 | 10549 | 11098 | 11595 | | 5175 | 12240 | 13198 | 13973 | | | |
| | IDENTITY | 43% | 100% | 43% | 33% | | 45% | 62% | 74% | 43% | | | |
| | COVERAGE | 92% | 100% | 92% | 96% | | 92% | 100% | 100% | 93% | | | |
| EFA101161 | SeqID | 10357 | 10551 | 11099 | | | 11994 | 12242 | 13199 | 13972 | | | |
| | IDENTITY | 39% | 100% | 35% | | | 37% | 69% | 66% | 36% | | | |
| | COVERAGE | 86% | 101% | 99% | | | 96% | 93% | 103% | 100% | | | |
| EFA101162 | SeqID | 10356 | 10555 | 11100 | 11441 | 11679 | 11993 | 12249 | 13200 | 13971 | | | |
| | IDENTITY | 58% | 100% | 58% | 59% | 59% | 57% | 78% | 84% | 58% | | | |
| | COVERAGE | 100% | 100% | 100% | 100% | 100% | 99% | 100% | 100% | 100% | | | |
| EFA101163 | SeqID | 10355 | 10557 | 11101 | 11594 | | 5174 | 12255 | 13201 | | | | |
| | IDENTITY | 66% | 100% | 68% | 60% | | 70% | 84% | 90% | | | | |
| | COVERAGE | 100% | 101% | 99% | 97% | | 100% | 101% | 100% | | | | |
| EFA101164 | SeqID | 10354 | 10558 | 11102 | 11593 | | 5173 | 12258 | 13202 | 13970 | | | |
| | IDENTITY | 55% | 100% | 58% | 47% | | 57% | 66% | 81% | 55% | | | |
| | COVERAGE | 91% | 101% | 91% | 91% | | 85% | 91% | 97% | 91% | | | |
| EFA101165 | SeqID | 10353 | 10559 | 11103 | 11592 | | 517212259 | 13203 13969 | | | | | |
| | IDENTITY | 59% | 100% | 60% | 52% | | 61% | 78% | 88% | 59% | | | |
| | COVERAGE | 95% | 100% | 95% | 99% | | 95% | 100% | 100% | 95% | | | |
| EFA101169 | SeqID | 10133 | 10574 | 11091 | | | 12025 | 12516 | | 13849 | | | |
| | IDENTITY | 27% | 100% | 28% | | | 26% | 41% | | 27% | | | |
| | COVERAGE | 93% | 100% | 97% | | | 94% | 100% | | 93% | | | |
| EFA101253 | SeqID | 10389 | 10852 | 11065 | 11551 | | 11838 | 13072 | 13457 | | | | |
| | IDENTITY | 43% | 100% | 42% | 31% | | 39% | 54% | 67% | | | | |
| | COVERAGE | 97% | 100% | 97% | 96% | | 99% | 97% | 99% | | | | |
| EFA101257 | SeqID | 10124 | 10917 | 10976 | 11484 | | 11914 | 12528 | 13357 | 14037 | | | |
| | IDENTITY | 40% | 100% | 39% | 39% | | 37% | 39% | 58% | 38% | | | |
| | COVERAGE | 99% | 100% | 99% | 101% | 97% | 97% | 100% | 101% | | | | |
| EFA101258 | SeqID | 10127 | 10918 | 10973 | 11513 | | 11892 | 12802 | 13358 | 13871 | | | |
| | IDENTITY | 40% | 100% | 40% | 39% | | 36% | 41% | 66% | 29% | | | |
| | COVERAGE | 97% | 101% | 96% | 95% | | 96% | 92% | 95% | 92% | | | |
| EFA101322 | SeqID | | 10620 | | | | | 12534 | 13328 | | | | |
| | IDENTITY | | 100% | | 66% | 65% | | | | | | | |
| | COVERAGE | | 100% | | | | | 86% | 86% | | | | |
| EFA101339 | SeqID | | 10743 | 11448 | | | 12326 | 13391 | | | | | |
| | IDENTITY | | 100% | 33% | | | 46% | 60% | | | | | |
| | COVERAGE | | 100% | | 97% | | | 98% | 98% | | | | |
| EFA101340 | SeqID | | 10745 | | | | | | | | | | |
| | IDENTITY | | 100% | | | | | | | | | | |
| | COVERAGE | | 102% | | | | | | | | | | |
| EFA101354 | SeqID | 10047 | 10648 | 11089 | 11608 | | 11935 | 12617 13345 | 13913 | | | | |
| | IDENTITY | 33% | 100% | 33% | 32% | | 34% | 38% | 36% | 32% | | | |
| | COVERAGE | 101% | 100% | 104% | 101% | 104% | 97% | 100% | 101% | | | | |
| EFA101370 | SeqID | | 10738 | | | | | 13126 | | | | | |
| | IDENTITY | | 100% | | | | | 31% | | | | | |
| | COVERAGE | | 101% | | | | | 98% | | | | | |
| EFA101403 | SeqID | | 10662 | | | | | 12941 | | | | | |
| | IDENTITY | | 100% | | | | | 34% | | | | | |
| | COVERAGE | | 100% | | | | | 100% | | | | | |
| EFA101404 | SeqID | 10210 | 10663 | 11214 | 11554 | | 11921 | 12135 13418 | 13925 | | | | |
| | IDENTITY | 29% | 100% | 28% | 39% | | 27% | 59% | 64% | 30% | | | |
| | COVERAGE | 99% | 100% | 102% | 98% | | 100% | 99% | 99% | 99% | | | |
| EFA101409 | SeqID | 10350 | 10524 | 11106 | 11437 | | 5170 | 12215 | 13207 | | | | |
| | IDENTITY | 54% | 100% | 58% | 44% | | 53% | 81% | 87% | | | | |
| | COVERAGE | 83% | 101% | 80% | 86% | | 91% | 91% | 91% | _______ | | | |
| EFA101410 | SeqID | 10349 | 10525 | 11107 | 11436 | | 5169 | 12216 | 13208 | 14108 | | | |
| | IDENTITY | 62% | 100% | 64% | 63% | | 66% | 90% | 90% | 62% | | | |
| | COVERAGE | 101% | 101% | 101% | 100% | 100% | 101% | 101% | 102% | | | | |
| EFA101411 | SeqID | 10348 | 10526 | 11108 | | | 5168 | 12217 | 13209 | 14107 | | | |
| | IDENTITY | 50% | 100% | 43% | | | 49% | 66% | 71% | 46% | | | |
| | COVERAGE | 97% | 101% | 97% | | | 93% | 96% | 99% | 97% | | | |
| EFA101412 | SeqID | 10347 | 10527 | 11109 | 11589 | 11654 | 5167 | 12218 | 13210 | 14106 | | | |
| | IDENTITY | 60% | 100% | 59% | 52% | 61% | 58% | 85% | 83% | 60% | | | |
| COVERAGE | 100% | 101% | 100% | 98% | 101% | 99% | 92% | 100% | 101% | | | | |
| EFA101414 | SeqID | 10345 | 10528 | 11111 | 11435 | | 5165 | 12219 | 13212 | 14104 | | | |
| | IDENTITY | 49% | 100% | 47% | 42% | | 46% | 79% | 81% | 49% | | | |
| | COVERAGE | 99% | 101% | 99% | 99% | | 100% | 101% | 101% | 101% | | | |
| EFA101415 | SeqID | 10344 | 10529 | 11112 | 11434 | | 5164 | 12220 | 13213 | 14103 | | | |
| IDENTITY | 47% | 100% | 50% | 39% | | 49% | 63% | 74% | 47% | | | | |
| COVERAGE | 98% | 101% | 98% | 100% | 98% | 101% | 101% | 98% | | | | | |
| EFA101416 | SeqID | 10343 | 10530 | 11113 | 11433 | | 5163 | 12221 | 13214 | 14102 | | | |
| | IDENTITY | 50% | 100% | 48% | 42% | | 52% | 68% | 82% | 51% | | | |
| | COVERAGE | 97% | 101% | 97% | 91% | | 94% | 99% | 101% | 98% | | | |
| EFA101417 | SeqID | 10342 | 10531 | 11114 | 11432 | | 5162 | 12222 | 13215 | 14101 | | | |
| | IDENTITY | 55% | 100% | 56% | 61% | | 52% | 72% | 85% | 55% | | | |
| | COVERAGE | 100% | 101% | 95% | 84% | | 92% | 95% | 94% | 100% | | | |
| EFA101424 | SeqID | 10220 | 10784 | 11276 | | 11765 | 11950 | 12350 | 13280 | 13934 | | | |
| | IDENTITY | 44% | 100% | 38% | | 34% | 36% | 65% | 79% | 41% | | | |
| | COVERAGE | 99% | 101% | 97% | | 73% | 78% | 101% | 99% | 99% | | | |
| EFA101425 | SeqID | 10240 | 10785 | 11275 | | | 11925 | 12351 13281 | 13863 | | | | |
| | IDENTITY | 49% | 100% | 50% | | | 39% | 63% | 78% | 47% | | | |
| | COVERAGE | 99% | 100% | 99% | | | 99% | 100% | 100% | 84% | | | |
| EFA101477 | SeqID | 10263 | 10861 | 10965 | 11562 | | 11948 | 13066 | 13525 | 14089 | | | |
| | IDENTITY | 52% | 100% | 50% | 41% | | 49% | 59% | 72% | 50% | | | |
| | COVERAGE | 91% | 100% | 95% | 91% | | 95% | 94% | 91% | 91% | | | |
| EFA101536 | SeqID | 10281 | 10823 | | | | | | | | | | |
| | IDENTITY | 30% | 100% | | | | | | | | | | |
| | COVERAGE | 86% | 100% | | | | | | | | | | |
| EFA101540 | SeqID | 10041 | 10487 | 11149 | 11456 | | 11941 | 12314 | 13438 | 13907 | | | |
| | IDENTITY | 51% | 100% | 50% | 50% | | 49% | 73% | 76% | 51% | | | |
| | COVERAGE | 92% | 100% | 90% | 86% | | 92% | 92% | 99% | 92% | | | |
| EFA101541 | SeqID | 10042 | 10488 | 11150 | 11620 | | 11940 | 12742 | 13437 | 13908 | | | |
| | IDENTITY | 41% | 100% | 45% | 35% | | 44% | 63% | 44% | 41% | | | |
| | COVERAGE | 100% | 100% | 98% | 121% | | 101% | 100% | 116% | 100% | | | |
| EFA101583 | SeqID | | 10593 | | | | | | | | | | |
| | IDENTITY | | 100% | | | | | | | | | | |
| | COVERAGE | | 100% | | | | | | | | | | |
| EFA101670 | SeqID | | 10511 | | | | | | | | | | |
| | IDENTITY | | 100% | | | | | | | | | | |
| | COVERAGE | | 100% | | | | | | | | | | |
| EFA101682 | SeqID | 10238 | 10789 | 11178 | 11517 | | 11829 | 12811 | 13673 | 13864 | | | |
| | IDENTITY | 45% | 100% | 45% | 40% | | 44% | 57% | 57% | 45% | | | |
| | COVERAGE | 97% | 100% | 98% | 95% | | 91% | 96% | 95% | 97% | | | |
| EFA101685 | SeqID | | 10791 | | 11369 | | 12022 | 12492 | 13368 | | | | |
| | IDENTITY | | 100% | | 47% | | 51% | 62% | 69% | | | | |
| | COVERAGE | | 100% | | 92% | | 98% | 97% | 99% | | | | |
| EFA101686 | SeqID | 10237 | 10940 | 10999 | 11325 | | 11901 | 12456 | 13455 | 13956 | | | |
| | IDENTITY | 39% | 100% | 37% | 37% | | 36% | 64% | 63% | 38% | | | |
| | COVERAGE | 99% | 100% | 99% | 99% | | 99% | 99% | 99% | 99% | | | |
| EFA101695 | SeqID | 10204 | 10629 | 11017 | 11479 | 11715 | 12106 | 12560 | 13284 | 13928 | | | |
| | IDENTITY | 34% | 100% | 32% | 34% | 31% | 35% | 51% | 75% | 34% | | | |
| | COVERAGE | 104% | 100% | 106% | 76% | 93% | 101% | 100% | 99% | 105% | | | |
| EFA101736 | SeqID | 10219 | 10775 | 11024 | | | 11924 | 12300 | 13340 | 13976 | | | |
| | IDENTITY | 33% | 100% | 29% | | | 27% | 35% | 32% | 28% | | | |
| | COVERAGE | 100% | 100% | 100% | | | 99% | 98% | 99% | 100% | | | |
| EFA101737 | SeqID | 10218 | 10778 | 11023 | | | 11923 | 12301 | 13341 | 13774 | | | |
| | IDENTITY | 39% | 100% | 37% | | | 42% | 43% | 43% | 58% | | | |
| | COVERAGE | 98% | 100% | 98% | | | 98% | 100% | 103% | 96% | | | |
| EFA101753 | SeqID | 10134 | 10552 | 11211 | | | 11895 | 12151 | 13693 | 13826 | | | |
| | IDENTITY | 36% | 100% | 37% | | | 36% | 50% | 50% | 37% | | | |
| | COVERAGE | 91% | 100% | 89% | | | 90% | 94% | 99% | 91% | | | |
| EFA101765 | SeqID | | 10587 | | | | | 13010 | 13353 | | | | |
| | IDENTITY | | 100% | | | | | 28% | 35% | | | | |
| | COVERAGE | | 100% | | 98% | 97% | | | | | | | |
| EFA101790 | SeqID | 10414 | 10803 | 11085 | | | 11915 | 12306 | | 13747 | | | |
| | IDENTITY | 42% | 100% | 41% | | | 39% | 46% | | 41% | | | |
| | COVERAGE | 101% | 100% | 101% | 101% | 101% | | 101% | | | | | |
| EFA101791 | SeqID | 10804 | | | | | 12359 | | | | | | |
| | IDENTITY | 100% | | | | | 37% | | | | | | |
| | COVERAGE | 101% | | | | | 77% | | | | | | |
| EFA101792 | SeqID | 10030 | 10805 | 11188 | 11458 | | 5187 | 12360 | 13333 | 14077 | | | |
| | IDENTITY | 31% | 100% | 32% | 27% | | 33% | 34% | 47% | 31% | | | |
| | COVERAGE | 98% | 100% | 96% | 98% | | 99% | 101% | 100% | 98% | | | |
| EFA101795 | SeqID | 10329 | 10922 | 11159 | 11322 | | 12062 | 12581 | 13363 | 13886 | | | |
| | IDENTITY | 34% | 100% | 36% | 36% | | 37% | 36% | 47% | 32% | | | |
| | COVERAGE | 98% | 101% | 98% | 99% | | 98% | 98% | 99% | 97% | | | |
| EFA101797 | SeqID | 10330 | 10924 | 11160 | 11321 | | 12063 | 13127 | 13364 | 13885 | | | |
| | IDENTITY | 53% | 100% | 52% | 49% | | 55% | 59% | 74% | 53% | | | |
| | COVERAGE | 98% | 100% | 98% | 98% | | 98% | 98% | 99% | 98% | | | |
| EFA101799 | SeqID | 10048 | 10926 | 11014 | 11339 | | 11934 | 12908 | 13366 | 13897 | | | |
| | IDENTITY | 53% | 100% | 55% | 49% | | 55% | 54% | 66% | 54% | | | |
| | COVERAGE | 97% | 100% | 97% | 94% | | 97% | 97% | 97% | 97% | | | |
| EFA101833 | SeqID | 10429 | 10720 | | 11335 | | 12039 | 12340 | 13451 | 14072 | | | |
| | IDENTITY | 31% | 100% | | 36% | | 35% | 51% | 59% | 31% | | | |
| | COVERAGE | 79% | 100% | | 92% | | 89% | 92% | 91% | 79% | | | |
| EFA101868 | SeqID | | 10829 | | | | | | | | | | |
| | IDENTITY | | 100% | | | | | | | | | | |
| | COVERAGE | | 100% | | | | | | | | | | |
| EFA101872 | SeqID | 10305 | 10815 | 11044 | 11343 | 11639 | 11797 | 12568 | 13288 | 13779 | | | |
| | IDENTITY | 62% | 100% | 62% | 38% | 61% | 60% | 93% | 92% | 62% | | | |
| | COVERAGE | 86% | 102% | 86% | 86% | 79% | 95% | 97% | 102% | 86% | | | |
| EFA101873 | SeqID | | 10816 | | | | 11796 | | | | | | |
| | IDENTITY | | 100% | | | | 36% | | | | | | |
| | COVERAGE | | 101% | | | | 94% | | | | | | |
| EFA101892 | SeqID | 10454 | 10506 | 11048 | 11281 | | 12005 | 12142 | 13190 | 14021 | | | |
| | IDENTITY | 47% | 100% | 47% | 41% | | 53% | 49% | 46% | 47% | | | |
| | COVERAGE | 100% | 101% | 100% | 97% | | 100% | 101% | 100% 100% | | | | |
| EFA101924 | SeqID | | 10891 | | 11532 | | | 12331 | 13463 | | | | |
| | IDENTITY | | 100% | | 36% | | | 65% | 65% | | | | |
| | COVERAGE | | 100% | | 101% | | | 100% | 94% | | | | |
| EFA101925 | SeqID | | 10893 | | | | | 12332 | | | | | |
| | IDENTITY | | 100% | | | | | 59% | | | | | |
| | COVERAGE | | 100% | | | | | 99% | | | | | |
| EFA101963 | SeqID | 10034 | 10848 | 11148 | 11536 | | 12006 | 12552 | 13648 | 13901 | | | |
| | IDENTITY | 48% | 100% | 47% | 49% | | 47% | 57% | 69% | 48% | | | |
| | COVERAGE | 105% | 100% | 105% | 99% | | 108% | 101% | 100% | 105% | | | |
| EFA102006 | SeqID | | 10580 | | | | 11830 | 12804 | 13315 | | | | |
| | IDENTITY | | 100% | | | | 33% | 42% | 43% | | | | |
| | COVERAGE | | 100% | 84% | 99% | 95% | | | | | | | |
| EFA102022 | SeqID | 10313 | 10881 | 11224 | 11502 | 11754 | 12051 | 12324 | 13485 | 13767 | | | |
| | IDENTITY | 53% | 100% | 53% | 51% | 54% | 55% | 78% | 78% | 52% | | | |
| | COVERAGE | 88% | 101% | 88% | 87% | 89% | 88% | 89% | 89% | 89% | | | |
| EFA102023 | SeqID | 10312 | 10882 | 10989 | 11576 | 11755 | 12050 | 12325 | 13699 | 13768 | | | |
| | IDENTITY | 51% | 100% | 50% | 38% | 50% | 50% | 63% | 70% | 50% | | | |
| | COVERAGE | 98% | 100% | 99% | 99% | 84% | 97% | 99% | 99% | 97% | | | |
| EFA102091 | SeqID | 10363 | 10481 | 11060 | 11568 | | 11858 | 12443 | 13233 | 13965 | | | |
| | IDENTITY | 60% | 100% | 61% | 63% | | 62% | 75% | 86% | 59% | | | |
| | COVERAGE | 101% | 100% | 101% | 100% | 101% | 100% | 100% | 101% | | | | |
| EFA102110 | SeqID | 10193 | 10841 | 11255 | | | 12082 | | 13430 | 13752 | | | |
| | IDENTITY | 32% | 100% | 34% | | | 34% | | 62% | 32% | | | |
| | COVERAGE | 103% | 100% | 94% | | | 100% | | 100% | 99% | | | |
| EFA102183 | SeqID | 10393 | 10952 | 11057 | 11330 | | 11774 | 12695 | 13420 | 13920 | | | |
| | IDENTITY | 55% | 100% | 54% | 50% | | 54% | 67% | 78% | 55% | | | |
| | COVERAGE | 84% | 100% | 86% | 85% | | 86% | 98% | 100% | 84% | | | |
| EFA102185 | SeqID | 10458 | 10950 | 11051 | 11421 | 11632 | 12075 | 12413 | 13501 | 13858 | | | |
| | IDENTITY | 27% | 100% | 29% | 29% | 28% | 29% | 63% | 73% | 27% | | | |
| | COVERAGE | 93% | 101% | 90% | 94% | 93% | 91% | 91% | 96% | 93% | | | |
| EFA102186 | SeqID | 10448 | 10949 | 10995 | 11579 | | | 12412 | 13543 | 13817 | | | |
| | IDENTITY | 29% | 100% | 29% | 27% | | | 53% | 60% | 30% | | | |
| | COVERAGE | 92% | 101% | 90% | 94% | | | 101% | 92% | 90% | | | |
| EFA102205 | SeqID | 10108 | 10769 | 10985 | 11375 | | | | 13375 | 13997 | | | |
| | IDENTITY | 46% | 100% | 38% | 56% | | | | 55% | 37% | | | |
| | COVERAGE | 71% | 102% | 82% | 73% | | | | 96% | 104% | | | |
| EFA102253 | SeqID | 10275 | 10727 | 11175 | 11320 | | 11933 | 12372 | 13376 | 13865 | | | |
| | IDENTITY | 53% | 100% | 55% | 48% | | 53% | 67% | 80% | 54% | | | |
| | COVERAGE | 100% | 100% | 101% | 101% | | 101% | 100% | 99% | 96% | | | |
| EFA102282 | SeqID | | 10729 | | | | | 12607 | 13424 | | | | |
| | IDENTITY | | 100% | | | | | 40% | 46% | | | | |
| | COVERAGE | | 101% | | | | | 81% | 76% | | | | |
| EFA102338 | SeqID | 10250 | 10651 | 11012 | 11488 | | 11954 | 12940 | 13272 | 13705 | | | |
| | IDENTITY | 39% | 100% | 38% | 35% | | 39% | 42% | 50% | 38% | | | |
| | COVERAGE | 95% | 100% | 92% | 86% | | 98% | 99% | 99% | 99% | | | |
| EFA102350 | SeqID | | 10632 | | | | | | | | | | |
| | IDENTITY | | 100% | | | | | | | | | | |
| | COVERAGE | | 101% | | | | | | | | | | |
| EFA102351 | SeqID | | 10634 | | | | | 12795 | 13406 | | | | |
| | IDENTITY | | 100% | | | | | 33% | 38% | | | | |
| | COVERAGE | | 100% | | | | | 97% | 101% | | | | |
| EFA102352 | SeqID | 10028 | 10635 | 11186 | 11328 | 11691 | 12011 | 12347 | 13409 | 14075 | | | |
| | IDENTITY | 40% | 100% | 39% | 35% | 40% | 39% | 51% | 55% | 40% | | | |
| | COVERAGE | 101% | 100% | 101% | 101% | 101% | 101% | 99% | 100% | 101% | | | |
| EFA102353 | SeqID | 10029 | 10636 | 11187 | 11329 | | 12010 | 12348 | 13398 | 14076 | | | |
| | IDENTITY | 32% | 100% | 34% | 28% | | 32% | 50% | 61% | 31% | | | |
| | COVERAGE | 99% | 100% | 99% | 83% | | 98% | 98% | 99% | 99% | | | |
| EFA102389 | SeqID | 10378 | 10904 | 11094 | | | 11781 | 12126 | 13263 | | | | |
| | IDENTITY | 41% | 100% | 42% | | | 40% | 54% | 52% | | | | |
| | COVERAGE | 97% | 100% | 83% | | | 98% | 82% | 100% | | | | |
| EFA102453 | SeqID | 10931 | 10995 | 11579 | 11762 | | 12412 | 13502 | 13819 | | | | |
| | IDENTITY | 100% | 29% | 33% | 33% | | 54% | 54% | 29% | | | | |
| | COVERAGE | 101% | 101% | 88% | 105% | | 101% | 101% | 96% | | | | |
| EFA102501 | SeqID | 10438 | 10626 | 11037 | 11410 | | 11997 | 12447 | 13187 | 14043 | | | |
| | IDENTITY | 45% | 100% | 44% | 40% | | 44% | 75% | 76% | 45% | | | |
| | COVERAGE | 112% | 100% | 111% | 114% | | 113% | 93% | 96% | 112% | | | |
| EFA102502 | SeqID | 10439 | 10627 | 11036 | 11410 | | 5179 | 12446 | 13646 | 14042 | | | |
| | IDENTITY | 47% | 100% | 46% | 52% | | 46% | 72% | 78% | 46% | | | |
| | COVERAGE | 114% | 100% | 117% | 79% | | 116% | 99% | 98% | 114% | | | |
| EFA102503 | SeqID | 10016 | 10643 | | 11446 | | 12027 | 12995 | 13481 | 13947 | | | |
| | IDENTITY | 45% | 100% | | 37% | | 43% | 61% | 65% | 41% | | | |
| | COVERAGE | 99% | 100% | | 101% | | 101% | 98% | 100% | 85% | | | |
| EFA102518 | SeqID | 10288 | 10647 | | | 11681 | | 12248 | 13229 | 13881 | | | |
| | IDENTITY | 33% | 100% | | | 50% | | 34% | 54% | 32% | | | |
| | COVERAGE | 105% | 100% | | | 71% | | 102% | 100% | 105% | | | |
| EFA102541 | SeqID | 10327 | 10602 | 11241 | 11471 | | 5188 | 12237 | 13356 | 13729 | | | |
| | IDENTITY | 59% | 100% | 59% | 49% | | 59% | 69% | 82% | 56% | | | |
| | COVERAGE | 77% | 101% | 77% | 73% | | 77% | 77% | 81% | 77% | | | |
| EFA102542 | SeqID | 10326 | 10603 | 11240 | 11288 | | 12016 | 12238 | 13361 | 13732 | | | |
| | IDENTITY | 75% | 100% | 70% | 67% | | 75% | 77% | 100% | 76% | | | |
| | COVERAGE | 95% | 105% | 95% | 100% | | 95% | 105% | 100% | 100% | | | |
| EFA102549 | SeqID | 10338 | 10538 | 11117 | 11428 | | 5159 | | | | | | |
| | IDENTITY | 63% | 100% | 63% | 71% | | 68% | | | | | | |
| | COVERAGE | 100% | 103% | 100% | 100% | | 100% | | | | | | |
| EFA102551 | SeqID | 10337 | 10539 | 11119 | 11427 | 11688 | 11990 | 12229 | 13221 | 14097 | | | |
| | IDENTITY | 59% | 100% | 61% | 58% | 30% | 62% | 75% | 81% | 58% | | | |
| | COVERAGE | 96% | 101% | 91% | 99% | 74% | 96% | 101% | 101% | 96% | | | |
| EFA102554 | SeqID | 10341 | 10532 | 11115 | | | 5161 | 12223 | 13216 | | | | |
| | IDENTITY | 45% | 100% | 40% | | | 42% | 62% | 63% | | | | |
| | COVERAGE | 93% | 102% | 93% | | | 97% | 102% | 100% | | | | |
| EFA102655 | SeqID | 10049 | 10733 | 11086 | 11305 | | 11813 | 12952 | 13228 | 13898 | | | |
| | IDENTITY | 47% | 100% | 47% | 42% | | 48% | 57% | 60% | 47% | | | |
| | COVERAGE | 97% | 100% | 99% | 99% | | 99% | 98% | 108% | 97% | | | |
| EFA102656 | SeqID | | 10734 | | | | | 12321 | 13668 | | | | |
| | IDENTITY | | 100% | | | | | 55% | 55% | | | | |
| | COVERAGE | | 100% | | | | | 100% | 100% | | | | |
| EFA102698 | SeqID | 10082 | 10909 | 10956 | | | 11807 | | | 14011 | | | |
| | IDENTITY | 56% | 100% | 60% | | | 31% | | | 55% | | | |
| | COVERAGE | 96% | 100% | 96% | | | 96% | | | 96% | | | |
| EFA102728 | SeqID | 10459 | 10948 | 11050 | 11420 | | 12074 | 12411 | 13503 | 13859 | | | |
| | IDENTITY | 51% | 100% | 53% | 52% | | 54% | 76% | 81% | 52% | | | |
| | COVERAGE | 89% | 101% | 89% | 73% | | 82% | 96% | 100% | 90% | | | |
| EFA102736 | SeqID | 10285 | 10556 | 11205 | 11300 | | 11943 | | 13401 | | | | |
| | IDENTITY | 53% | 100% | 52% | 44% | | 51% | | 71% | | | | |
| | COVERAGE | 98% | 100% | 100% | 98% | | 100% | | 99% | | | | |
| EFA102764 | SeqID | 10201 | 10478 | 11054 | | | | 12590 | 13425 | 13822 | | | |
| | IDENTITY | 72% | 100% | 56% | | | | 68% | 80% | 71% | | | |
| | COVERAGE | 99% | 100% | 99% | | | | 99% | 100% | 99% | | | |
| EFA102774 | SeqID | 10142 | 10896 | 11261 | 11362 | | 12040 | 12150 | 13235 | 13978 | | | |
| | IDENTITY | 50% | 100% | 52% | 52% | | 51% | 68% | 74% | 50% | | | |
| | COVERAGE | 96% | 100% | 96% | 94% | | 95% | 98% | 97% | 96% | | | |
| EFA102780 | SeqID | 10395 | 10908 | 11167 | 11616 | | 11772 | 12701 | 13552 | | | | |
| | IDENTITY | 49% | 100% | 46% | 37% | | 51% | 51% | 46% | | | | |
| | COVERAGE | 77% | 100% | 76% | 77% | | 75% | 101% | 98% | | | | |
| EFA102788 | SeqID | 10176 | 10661 | 11223 | 11297 | | 11882 | 12630 | 13303 | 13941 | | | |
| | IDENTITY | 59% | 100% | 61% | 54% | | 63% | 70% | 81% | 59% | | | |
| | COVERAGE | 94% | 101% | 93% | 97% | | 94% | 93% | 96% | 94% | | | |
| EFA102802 | SeqID | 10274 | 10854 | 11154 | 11298 | | 11932 | 13128 | 13313 | 13866 | | | |
| | IDENTITY | 66% | 100% | 64% | 58% | | 64% | 74% | 83% | 65% | | | |
| | COVERAGE | 99% | 100% | 100% | 96% | | 100% | 100% | 100% | 99% | | | |
| EFA102813 | SeqID | 10191 | 10878 | 11005 | 11347 | | 11815 | 12816 | 13492 | 13754 | | | |
| | IDENTITY | 54% | 100% | 53% | 51% | | 52% | 64% | 65% | 53% | | | |
| | COVERAGE | 100% | 100% | 100% | 99% | | 99% | 99% | 99% | 100% | | | |
| EFA102915 | SeqID | 10297 | 10640 | 10964 | 11323 | | 11783 | 13090 | 13664 | 13737 | | | |
| | IDENTITY | 27% | 100% | 32% | 30% | | 31% | 50% | 52% | 28% | | | |
| | COVERAGE | 100% | 100% | 100% | 90% | | 100% | 98% | 99% | 100% | | | |
| EFA103021 | SeqID | 10434 | 10612 | 11039 | 11413 | | 11999 | 12451 | 13517 | | | | |
| | IDENTITY | 65% | 100% | 66% | 60% | | 62% | 86% | 86% | | | | |
| | COVERAGE | 101% | 101% | 101% | 99% | | 101% | 101% | 99% | | | | |
| EFA103033 | SeqID | 10221 | 10681 | 11210 | 11607 | 11668 | 11801 | 12289 | 13191 | 14027 | | | |
| | IDENTITY | 42% | 100% | 40% | 29% | 42% | 39% | 49% | 56% | 30% | | | |
| | COVERAGE | 91% | 100% | 93% | 98% | 94% | 91% | 93% | 92% | 93% | | | |
| EFA103038 | SeqID | 10435 | 10613 | 11038 | 11412 | | 11998 | 12784 | 13397 | 14046 | | | |
| | IDENTITY | 54% | 100% | 52% | 56% | | 51% | 73% | 73% | 53% | | | |
| | COVERAGE | 99% | 100% | 100% | 99% | | 100% | 100% | 100% | 99% | | | |
| EFA103039 | SeqID | 10293 | 10850 | 11041 | 11482 | 11728 | 11793 | 12541 | 13377 | 13741 | | | |
| | IDENTITY | 45% | 100% | 46% | 44% | 40% | 46% | 73% | 69% | 45% | | | |
| | COVERAGE | 99% | 100% | 101% | 98% | 99% | 99% | 102% | 101% | 99% | | | |
| EFA103062 | SeqID | 10437 | 10615 | 11072 | 11572 | | 5180 | 12449 | 13247 | 14044 | | | |
| | IDENTITY | 59% | 100% | 64% | 54% | | 65% | 64% | 68% | 59% | | | |
| | COVERAGE | 101% | 101% | 102% | 102% | | 101% | 99% | 101% | 102% | | | |
| EFA103081 | SeqID | 10262 | 10862 | 10984 | 11403 | | 11947 | | 13415 | 14090 | | | |
| | IDENTITY | 41% | 100% | 41% | 40% | | 41% | | 74% | 40% | | | |
| | COVERAGE | 85% | 101% | 83% | 82% | | 80% | | 95% | 85% | | | |
| EFA103174 | SeqID | 10251 | 10689 | 10969 | 11370 | | 11955 | 12600 | 13518 | 13703 | | | |
| | IDENTITY | 32% | 100% | 32% | 37% | | 33% | 63% | 77% | 33% | | | |
| | COVERAGE | 93% | 100% | 94% | 95% | | 96% | 100% | 100% | 92% | | | |
| EFA103210 | SeqID | 10071 | 10688 | 11019 | 11371 | | 11850 | 12601 | 13319 | 13945 | | | |
| | IDENTITY | 56% | 100% | 63% | 39% | | 57% | 79% | 76% | 57% | | | |
| | COVERAGE | 97% | 101% | 98% | 99% | | 97% | 99% | 101% | 99% | | | |
| EFA103268 | SeqID | 10365 | 10479 | 11062 | 11409 | | 5178 | 12445 | 13231 | 13967 | | | |
| | IDENTITY | 69% | 100% | 70% | 68% | | 70% | 83% | 93% | 70% | | | |
| | COVERAGE | 100% | 101% | 100% | 100% | 99% | 101% | 101% | 101% | | | | |
| EFA103295 | SeqID | 10319 | 10633 | 11140 | 11493 | | 12029 | 12640 | 13320 | 13771 | | | |
| | IDENTITY | 66% | 100% | 58% | 58% | | 70% | 79% | 86% | 60% | | | |
| | COVERAGE | 77% | 101% | 85% | 85% | | 77% | 100% | 96% | 92% | | | |
| EFA103348 | SeqID | | 10873 | 10983 | 11402 | | 11946 | | | | | | |
| | IDENTITY | | 100% | 39% | 59% | | 39% | | | | | | |
| | COVERAGE | | 103% | 82% | 85% | | 82% | | | | | | |
| EFA103365 | SeqID | 10360 | 10533 | 11096 | 11443 | 11643 | 5177 | 12224 | 13196 | 13975 | | | |
| | IDENTITY | 57% | 100% | 58% | 53% | 58% | 58% | 82% | 82% | 58% | | | |
| | COVERAGE | 100% | 101% | 100% | 97% | 100% | 100% | 88% | 101% | 100% | | | |
| EFA103375 | SeqID | 10177 | 10660 | 11222 | 11296 | | 5120 | 12628 | 13302 | | | | |
| | IDENTITY | 50% | 100% | 52% | 36% | | 50% | 66% | 78% | | | | |
| | COVERAGE | 82% | 102% | 82% | 97% | | 94% | 102% | 102% | | | | |
| EFA103504 | SeqID | 10320 | 10671 | 11141 | 11492 | | 12030 | 12638 | 13322 | 13766 | | | |
| | IDENTITY | 42% | 100% | 45% | 41% | | 48% | 63% | 81% | 41% | | | |
| | COVERAGE | 97% | 101% | 97% | 96% | | 97% | 98% | 100% | 100% | | | |
| EFA103508 | SeqID | | 10672 | | | | | | 13321 | | | | |
| | IDENTITY | | 100% | | | | | | 30% | | | | |
| | COVERAGE | | 100% | | | | | | 80% | | | | |
| EFA103571 | SeqID | 10335 | 10879 | 11121 | 11425 | | 11988 | 12578 | 13240 | 14095 | | | |
| | IDENTITY | 45% | 100% | 47% | 48% | | 47% | 67% | 68% | 45% | | | |
| | COVERAGE | 102% 100% | 102% | 103% | | 102% | 99% | 100% | 102% | | | | |
| EFA103786 | SeqID | | 10806 | | | | | 12361 | | | | | |
| | IDENTITY | | 100% | | | | | 59% | | | | | |
| | COVERAGE | | 100% | | | | | 94% | | | | | |
| SAU100040 | SeqID | | | | | | | 12533 | | | | | |
| | IDENTITY | | | | | | | 100% | | | | | |
| | COVERAGE | | | | | | | 101% | | | | | |
| SAU100053 | SeqID | 10366 | 10504 | 11075 | 11376 | 11723 | 11855 | 12143 | 13318 | 13814 | | | |
| | IDENTITY | 32% | 46% | 30% | 32% | 33% | 33% | 100% | 48% | 32% | | | |
| | COVERAGE | 97% | 100% | 99% | 81% | 84% | 81% | 100% | 100% | 97% | | | |
| SAU100056 | SeqID | | 10930 | | | | | 12577 | 13477 | | | | |
| | IDENTITY | | 39% | | | | | 100% | 33% | | | | |
| | COVERAGE | | 98% | | | | | 100% | 100% | | | | |
| SAU100059 | SeqID | 10213 | 10598 | 11161 | 11528 | 11750 | 12064 | 12652 | 13433 | 13929 | | | |
| | IDENTITY | 28% | 70% | 26% | 26% | 27% | 28% | 100% | 25% | 28% | | | |
| | COVERAGE | 71% | 97% | 95% | 95% | 71% | 96% | 100% | 95% | 71% | | | |
| SAU100062 | SeqID | 10430 | 10618 | 10998 | 11603 | 11739 | | 12309 | 13294 | 14040 | | | |
| | IDENTITY | 27% | 52% | 29% | 29% | 31% | | 100% | 53% | 28% | | | |
| | COVERAGE | 103% | 96% | 103% | 77% | 76% | | 100% | 97% | 102% | | | |
| SAU100077 | SeqID | | 10565 | | | | | 12520 | 13464 | | | | |
| | IDENTITY | | 64% | | | | | 100% | 62% | | | | |
| | COVERAGE | | 102% | | | | | 100% | 102% | | | | |
| SAU100112 | SeqID | 10059 | | | 11477 | 11702 | 12096 | 12634 | | 13895 | | | |
| | IDENTITY | 49% | | | 52% | 53% | 46% | 100% | | 49% | | | |
| | COVERAGE | 97% | | | 100% | 77% | 100% | 100% | | 97% | | | |
| SAU100114 | SeqID | 10152 | 10515 | 11279 | 11302 | | 11851 | 12535 | 13387 | 13824 | | | |
| | IDENTITY | 44% | 51% | 43% | 45% | | 43% | 100% | 25% | 43% | | | |
| | COVERAGE | 98% | 98% | 98% | 98% | | 98% | 100% | 102% | 98% | | | |
| SAU100118 | SeqID | | 10903 | | | | 11828 | 12125 | 13262 | | | | |
| | IDENTITY | | 41% | | | | 27% | 100% | 37% | | | | |
| | COVERAGE | | 101% | | | | 100% | 100% | 101% | | | | |
| SAU100123 | SeqID | 10258 | 10628 | 11134 | 11489 | | 5192 | 12526 | 13421 | 14088 | | | |
| | IDENTITY | 52% | 43% | 53% | 47% | | 52% | 100% | 45% | 52% | | | |
| | COVERAGE | 98% | 100% | 97% | 96% | | 98% | 100% | 82% | 98% | | | |
| SAU100131 | SeqID | 10466 | | 11274 | | | 11960 | 12517 | | 13854 | | | |
| | IDENTITY | 35% | | 33% | | | 40% | 100% | | 35% | | | |
| | COVERAGE | 71% | | 97% | | | 70% | 100% | | 71% | | | |
| SAU100133 | SeqID | 10311 | 10493 | 10990 | 11308 | 11703 | 11885 | 12574 | 13412 | 13769 | | | |
| | IDENTITY | 34% | 44% | 34% | 33% | 30% | 31% | 100% | 43% | 34% | | | |
| | COVERAGE | 79% | 99% | 80% | 78% | 82% | 79% | 100% | 99% | 79% | | | |
| SAU100139 | SeqID | 10355 | 10557 | 11101 | 11594 | | 5174 | 12255 | 13201 | | | | |
| | IDENTITY | 65% | 84% | 66% | 64% | | 63% | 100% | 86% | | | | |
| | COVERAGE | 85% | 86% | 81% | 83% | | 84% | 101% | 85% | | | | |
| SAU100140 | SeqID | 10354 | 10558 | 11102 | 11440 | | 5173 | 12258 | 13202 | 13970 | | | |
| | IDENTITY | 54% | 66% | 54% | 40% | | 48% | 100% | 63% | 54% | | | |
| | COVERAGE | 93% | 91% | 93% | 94% | | 93% | 101% | 91% | 93% | | | |
| SAU100141 | SeqID | 10353 | 10559 | 11103 | 11592 | | 5172 | 12259 | 13203 | 13969 | | | |
| | IDENTITY | 55% | 78% | 58% | 54% | | 57% | 100% | 74% | 55% | | | |
| | COVERAGE | 96% | 101% | 96% | 96% | | 96% | 100% | 100% | 96% | | | |
| SAU100157 | SeqID | 10364 | 10480 | 11061 | 11408 | 11659 | 11996 | 12444 | 13232 | 13966 | | | |
| | IDENTITY | 60% | 78% | 60% | 55% | 62% | 57% | 100% | 77% | 60% | | | |
| | COVERAGE | 100% | 101% | 100% | 99% | 88% | 100% | 101% | 101% | 101% | | | |
| SAU100158 | SeqID | 10363 | 10481 | 11060 | 11568 | | 11858 | 12443 | 13233 | 13965 | | | |
| | IDENTITY | 60% | 75% | 59% | 63% | | 59% | 100% | 77% | 58% | | | |
| | COVERAGE | 98% | 97% | 98% | 97% | | 98% | 100% | 97% | 99% | | | |
| SAU100162 | SeqID | 10069 | 10630 | 11239 | 11382 | | 11971 | 12583 | 13597 | 14084 | | | |
| | IDENTITY | 43% | 49% | 44% | 37% | | 43% | 100% | 46% | 43% | | | |
| | COVERAGE | 92% | 89% | 88% | 80% | | 83% | 100% | 89% | 93% | | | |
| SAU100175 | SeqID | 10250 | 10651 | 11012 | | | 11954 | 12582 | 13272 | 13705 | | | |
| | IDENTITY | 34% | 42% | 38% | | | 34% | 100% | 42% | 35% | | | |
| | COVERAGE | 98% | 100% | 93% | | | 93% | 100% | 102% | 99% | | | |
| SAU100182 | SeqID | | | | | | | 12362 | | | | | |
| | IDENTITY | | | | | | | 100% | | | | | |
| | COVERAGE | | | | | | | 101% | | | | | |
| SAU100186 | SeqID | 10043 | 10489 | 11124 | 11423 | | 11939 | 12317 | 13355 | 13909 | | | |
| | IDENTITY | 46% | 61% | 44% | 46% | | 45% | 100% | 54% | 45% | | | |
| | COVERAGE | 99% | 99% | 99% | 98% | | 100% | 101% | 99% | 101% | | | |
| SAU100198 | SeqID | | | | 11445 | | | 12120 | 13414 | | | | |
| | IDENTITY | | | | 29% | | | 100% | 29% | | | | |
| | COVERAGE | | | | 78% | | | 101% | 79% | | | | |
| SAU100227 | SeqID | | 10765 | | | | | 12525 | | | | | |
| | IDENTITY | | 36% | | | | | 100% | | | | | |
| | COVERAGE | | 100% | | | | | 100% | | | | | |
| SAU100242 | SeqID | 10097 | | 11201 | | | 11836 | 12336 | | 14056 | | | |
| | IDENTITY | 65% | | 62% | | | 65% | 100% | | 65% | | | |
| | COVERAGE | 94% | | 96% | | | 95% | 100% | 94% | | | | |
| SAU100246 | SeqID | | 10821 | | | | | 12496 | 13490 | | | | |
| | IDENTITY | | 35% | | | | | 100% | 38% | | | | |
| | COVERAGE | | 101% | | | | | 101% | 93% | | | | |
| SAU100251 | SeqID | | | | | | 12363 | | | | | | |
| | IDENTITY | | | | | | 100% | | | | | | |
| | COVERAGE | | | | | | | 100% | | | | | |
| SAU100265 | SeqID | 10469 | | | | | | 12122 | | | | | |
| | IDENTITY | 37% | | | | | | 100% | | | | | |
| | COVERAGE | 88% | | | | | | 100% | | | | | |
| SAU100266 | SeqID | | | | | | | 12256 | | | | | |
| | IDENTITY | | | | | | | 100% | | | | | |
| | COVERAGE | | | | | | | 101% | | | | | |
| SAU100272 | SeqID | | 10617 | | | | | 12141 | | | | | |
| | IDENTITY | | 26% | | | | | 100% | | | | | |
| | COVERAGE | | 104% | | | | | 100% | | | | | |
| SAU100275 | SeqID | 10041 | 10487 | 11149 | 11621 | | 11941 | 12314 | 13438 | 13907 | | | |
| | IDENTITY | 52% | 73% | 47% | 51% | | 51% | 100% | 65% | 51% | | | |
| | COVERAGE | 88% | 94% | 93% | 98% | | 90% | 100% | 98% | 88% | | | |
| SAU100300 | SeqID | 10434 | 10612 | 11039 | 11413 | | 11999 | 12451 | 13517 | | | | |
| | IDENTITY | 67% | 86% | 68% | 63% | | 65% | 100% | 82% | | | | |
| | COVERAGE | 99% | 99% | 99% | 97% | | 99% | 101% | 97% | | | | |
| SAU100301 | SeqID | 10433 | 10624 | 11083 | 11414 | | 12000 | 12452 | 13168 | | | | |
| | IDENTITY | 41% | 58% | 41% | 35% | | 42% | 100% | 51% | | | | |
| | COVERAGE | 99% | 98% | 102% | 96% | | 98% | 101% | 97% | | | | |
| SAU100302 | SeqID | 10432 | | 11082 | | | 12001 | 12453 | | | | | |
| | IDENTITY | 25% | | 34% | | | 31% | 100% | | | | | |
| | COVERAGE | 92% | | 93% | | | 103% | 102% | | | | | |
| SAU100305 | SeqID | 10311 | 10774 | 10990 | | | 11885 | 12397 | 13491 | 13769 | | | |
| | IDENTITY | 40% | 50% | 38% | | | 40% | 100% | 49% | 40% | | | |
| | COVERAGE | 94% | 99% | 94% | | | 92% | 100% | 101% | 94% | | | |
| SAU100307 | SeqID | 10392 | 10725 | 10954 | | 11685 | | 12313 | 13252 | 13919 | | | |
| | IDENTITY | 28% | 32% | 29% | | 28% | | 100% | 29% | 28% | | | |
| | COVERAGE | 99% | 100% | 99% | | 99% | | 100% | 99% | 99% | | | |
| SAU100308 | SeqID | 10013 | 10814 | 10963 | | | | 12312 | 13244 | 13711 | | | |
| | IDENTITY | 26% | 44% | 30% | | | | 100% | 40% | 27% | | | |
| | COVERAGE | 90% | 86% | 86% | | | | 100% | 92% | 90% | | | |
| SAU100313 | SeqID | | 10757 | | | | | 12661 | 13293 | | | | |
| | IDENTITY | | 46% | | | | | 100% | 43% | | | | |
| | COVERAGE | | 99% | | | | | 100% | 100% | | | | |
| SAU100315 | SeqID | 10419 | 10802 | 11136 | 11326 | 11727 | 12087 | 12358 | 13521 | 13791 | | | |
| | IDENTITY | 54% | 73% | 53% | 53% | 55% | 53% | 100% | 74% | 54% | | | |
| | COVERAGE | 96% | 96% | 96% | 96% | 82% | 97% | 100% | 91% | 96% | | | |
| SAU100323 | SeqID | 10216 | 10855 | | | | | 12575 | | 13933 | | | |
| | IDENTITY | 32% | 71% | | | | | 100% | | 34% | | | |
| | COVERAGE | 88% | 99% | | | | | 100% | | 88% | | | |
| SAU100347 | SeqID | 10895 | 10961 | | | 12077 | 12334 | 13206 | | | | | |
| | IDENTITY | 44% | 30% | | | 30% | 100% | 42% | | | | | |
| | COVERAGE | 106% | 84% | | | 100% | 100% | 100% | | | | | |
| SAU100355 | SeqID | | 10683 | | | | | 12155 | 13300 | | | | |
| | IDENTITY | | 42% | | | | | 100% | 31% | | | | |
| | COVERAGE | | 93% | | | | | _100% | 109% | | | | |
| SAU100359 | SeqID | | 10757 | | | | | 12239 | 13293 | | | | |
| | IDENTITY | | 52% | | | | | 100% | 43% | | | | |
| | COVERAGE | | 97% | | | | | 100% | 99% | | | | |
| SAU100381 | SeqID | 10411 | 10674 | | | | 11903 | 12276 | | 14031 | | | |
| | IDENTITY | 28% | 29% | 33% | | | | 100% | | 28% | | | |
| | COVERAGE | 101% | 99% | | | | 92% | 100% | | 101% | | | |
| SAU100389 | SeqID | 10473 | 10737 | | 11374 | | | 12279 | 13344 | | | | |
| | IDENTITY | 27% | 50% | | 41% | | | 100% | 27% | | | | |
| | COVERAGE | 75% | 95% | | 99% | | | 100% | 71% | | | | |
| SAU100401 | SeqID | 10090 | 10706 | 10980 | | 11641 | | 12576 | | 14053 | | | |
| | IDENTITY | 31% | 30% | 27% | | 33% | | 100% | | 31% | | | |
| | COVERAGE | 95% | 99% | 95% | | 95% | | 101% | | 99% | | | |
| SAU100412 | SeqID | 10102 | 10563 | 11194 | 11360 | | 5150 | 12197 | 13468 | | | | |
| | IDENTITY | 31% | 42% | 30% | 33% | | 35% | 100% | 40% | | | | |
| | COVERAGE | 74% | 100% | 80% | 74% | | 73% | 100% | 97% | | | | |
| SAU100414 | SeqID | 10453 | 10556 | 11205 | 11300 | | 11943 | 12148 | 13401 | 13872 | | | |
| | IDENTITY | 60% | 80% | 61% | 60% | | 67% | 100% | 76% | 60% | | | |
| | COVERAGE | 96% | 99% | 98% | 99% | | 91% | 101% | 96% | 96% | | | |
| SAU100432 | SeqID | 10436 | 10614 | 11071 | 11411 | | 5181 | 12450 | 13246 | 14045 | | | |
| | IDENTITY | 34% | 60% | 33% | 31% | | 39% | 100% | 55% | 31% | | | |
| | COVERAGE | 98% | 98% | 100% | 95% | | 99% | 101% | 98% | 98% | | | |
| SAU100433 | SeqID | 10437 | 10615 | 11072 | 11572 | | 5180 | 12449 | 13247 | 14044 | | | |
| | IDENTITY | 58% | 64% | 63% | 57% | | 58% | 100% | 69% | 58% | | | |
| | COVERAGE | 97% | 99% | 98% | 99% | | 98% | 101% | 99% | 98% | | | |
| SAU100436 | SeqID | | 10569 | | | | | 12154 | 13393 | | | | |
| | IDENTITY | | 27% | | | | | 100% | 27% | | | | |
| | COVERAGE | | 100% | | | | | 100% | 100% | | | | |
| SAU100443 | SeqID | 10272 | 10894 | 11081 | | | 11930 | 12333 | 13515 | 13869 | | | |
| | IDENTITY | 40% | 52% | 39% | | | 38% | 100% | 45% | 40% | | | |
| | COVERAGE | 92% | 100% | 96% | | | 92%100% | 100% | 92% | | | | |
| SAU100444 | SeqID | 10440 | 10583 | 11016 | 11540 | | 11967 | 12392 | 13403 | 14041 | | | |
| | IDENTITY | 29% | 30% | 41% | 41% | | 28% | 100% | 52% | 29% | | | |
| | COVERAGE | 75% | 88% | 94% | 90% | | 81%100% | 91% | 75% | | | | |
| SAU100475 | SeqID | | 10927 | | | | 11911 | 12337 | | | | | |
| | IDENTITY | | 33% | | | | 30% | 100% | | | | | |
| | COVERAGE | | 101% | | | | 101% 100% | | | | | | |
| SAU100478 | SeqID | | | 11273 | | | | 12605 | | | | | |
| | IDENTITY | | | 25% | | | | 100% | | | | | |
| | COVERAGE | | | 96% | | | | 100% | | | | | |
| SAU100489 | SeqID | 10332 | 10685 | 11074 | 11580 | 11729 | 11778 | 12566 | 13298 | 14100 | | | |
| | IDENTITY | 33% | 33% | 31% | 34% | 34% | 29% | 100% | 34% | 33% | | | |
| | COVERAGE | 101% | 102% | 99% | 94% | 101% | 99% | 100% | 97% | 94% | | | |
| SAU100496 | SeqID | | 10744 | | | | | 12484 | | | | | |
| | IDENTITY | | 40% | | | | | 100% | | | | | |
| | COVERAGE | | 80% | | | | | 100% | | | | | |
| SAU100497 | SeqID | 10245 | 10709 | 11171 | 11395 | | 11792 | 12140 | | 13740 | | | |
| | IDENTITY | 46% | 59% | 49% | 44% | | 48% | 100% | | 45% | | | |
| | COVERAGE | 99% | 101% | 99% | 100% | | 99% | 100% | | 100% | | | |
| SAU100514 | SeqID | 10215 | | | 11388 | | 12036 | 12626 | | 13932 | | | |
| | IDENTITY | 52% | | | 34% | | 51% | 100% | | 51% | | | |
| | COVERAGE | 93% | | | 95% | | 98% | 100% | | 95% | | | |
| SAU100521 | SeqID | 10251 | | 10969 | 11370 | | 11955 | 12600 | | 13703 | | | |
| | IDENTITY | 43% | | 39% | 34% | | 39% | 100% | | 42% | | | |
| | COVERAGE | 104% | 108% | 103% | 103% | | 100% | 104% | | | | | |
| SAU100522 | SeqID | 10114 | | 11206 | | 11680 | 11904 | 12599 | | 14007 | | | |
| | IDENTITY | 36% | | 34% | | 30% | 36% | 100% | | 35% | | | |
| | COVERAGE | 91% | | 89% | | 80% | 90% | 100% | | 91% | | | |
| SAU100527 | SeqID | 10298 | 10721 | 10996 | | | 11782 | 12341 | 13452 | 13736 | | | |
| | IDENTITY | 44% | 48% | 42% | | | 41% | 100% | 43% | 45% | | | |
| | COVERAGE | 98% | 97% | 99% | | | 98% | 101% | 98% | 97% | | | |
| SAU100528 | SeqID | | 10521 | | | | | 12507 | 13470 | | | | |
| | IDENTITY | | 30% | | | | | 100% | 33% | | | | |
| | COVERAGE | | 83% | | | | | 101% | 71% | | | | |
| SAU100532 | SeqID | 10235 | 10645 | 11128 | 11389 | | | 12580 | 13193 | 13744 | | | |
| | IDENTITY | 39% | 47% | 29% | 34% | | | 100% | 40% | 31% | | | |
| | COVERAGE | 101% | 100% | 72% | 90% | | | 100% | 97% | 72% | | | |
| SAU100542 | SeqID | 10371 | 11070 | 11422 | | 12017 | 12532 | 13444 | 13806 | | | | |
| | IDENTITY | 52% | 51% | 46% | | 31% | 100% | 35% | 52% | | | | |
| | COVERAGE | 100% | 98% | 98% | | 102% | 100% | 102% | 100% | | | | |
| SAU100546 | SeqID | 10359 | 11097 | 11596 | | 5176 | 12235 | 13197 | 13974 | | | | |
| | IDENTITY | 43% | 46% | 34% | | 47% | 100% | 66% | 46% | | | | |
| | COVERAGE | 97% | 97% | 90% | | 99% | 100% | 99% | 91% | | | | |
| SAU100547 | SeqID | 10358 | 10549 | 11098 | 11595 | | 5175 | 12240 | 13198 | 13973 | | | |
| | IDENTITY | 41% | 62% | 39% | 40% | | 46% | 100% | 63% | 41% | | | |
| | COVERAGE | 92% | 100% | 97% | 96% | | 97% | 100% | 100% | 93% | | | |
| SAU100557 | SeqID | | 10928 | | | | | 12565 | 13651 | | | | |
| | IDENTITY | | 50% | | | | | 100% | 49% | | | | |
| | COVERAGE | | 99% | | | | | 100% | 99% | | | | |
| SAU100582 | SeqID | | | | | | | 12503 | | | | | |
| | IDENTITY | | | | | | | 100% | | | | | |
| | COVERAGE | | | | | | | 100% | | | | | |
| SAU100590 | SeqID | | | | | | | 12121 | | | | | |
| | IDENTITY | | | | | | | 100% | | | | | |
| | COVERAGE | | | | | | | 100% | | | | | |
| SAU100595 | SeqID | 10051 | 10832 | | 11464 | | 12109 | 12547 | 13174 | 13722 | | | |
| | IDENTITY | 47% | 66% | | 42% | | 50% | 100% | 46% | 42% | | | |
| | COVERAGE | 88% | 89% | | 89% | | 93% | 100% | 90% | 91% | | | |
| SAU100596 | SeqID | 10050 | 10833 | 11067 | 11624 | 11656 | 12110 | 12548 | 13173 | 13720 | | | |
| | IDENTITY | 36% | 50% | 31% | 41% | 38% | 42% | 100% | 30% | 32% | | | |
| | COVERAGE | 99% | 99% | 100% | 92% | 89% | 95% | 100% | 106% | 95% | | | |
| SAU100601 | SeqID | | | | | | | 12616 | | | | | |
| | IDENTITY | | | | | | | 100% | | | | | |
| | COVERAGE | | | | | | | 100% | | | | | |
| SAU100608 | SeqID | 10032 | 10870 | 11190 | 11349 | | 12008 | 12293 | 13507 | 14079 | | | |
| | IDENTITY | 30% | 61% | 29% | 29% | | 34% | 100% | 50% | 28% | | | |
| | COVERAGE | 102% | 96% | 100% | 98% | | 87% | 100% | 96% | 104% | | | |
| SAU100610 | SeqID | | | | | | | 12294 | | | | | |
| | IDENTITY | | | | | | | 100% | | | | | |
| | COVERAGE | | | | | | | 100% | | | | | |
| SAU100613 | SeqID | 10378 | 10904 | 11094 | | | 11781 | 12126 | 13589 | | | | |
| | IDENTITY | 44% | 54% | 43% | | | 46% | 100% | 49% | | | | |
| | COVERAGE | 91% | 88% | 93% | | | 73% | 100% | 89% | | | | |
| SAU100617 | SeqID | | 10502 | | | | | 12295 | 13314 | | | | |
| | IDENTITY | | 26% | | | | | 100% | 25% | | | | |
| | COVERAGE | | 91% | | | | | 100% | 91% | | | | |
| SAU100633 | SeqID | 10079 | 10589 | | | 11698 | 5107 | 12515 | 13644 | 13724 | | | |
| | IDENTITY | 27% | 42% | | | 25% | 29% | 100% | 35% | 26% | | | |
| | COVERAGE | 92% | 103% | | | 89% | 101% | 100% | 105% | 103% | | | |
| SAU100646 | SeqID | 10051 | 10570 | | 11464 | | 12109 | 12168 | 13174 | 14109 | | | |
| | IDENTITY | 50% | 48% | | 46% | | 49% | 100% | 42% | 50% | | | |
| | COVERAGE | 95% | 94% | | 97% | | 95% | 100% | 95% | 96% | | | |
| SAU100658 | SeqID | 10322 | 10813 | 11177 | 11351 | | 12018 | 12388 | 13186 | 13733 | | | |
| | IDENTITY | 49% | 59% | 49% | 46% | | 48% | 100% | 58% | 49% | | | |
| | COVERAGE | 100% | 100% | 100% | 100% | | 100% | 100% | 100% | 100% | | | |
| SAU100659 | SeqID | 10045 | 10923 | 11174 | 11601 | | 11937 | 12390 | 13616 | 13911 | | | |
| | IDENTITY | 47% | 54% | 45% | 40% | | 46% | 100% | 56% | 44% | | | |
| | COVERAGE | 92% | 92% | 95% | 103% | | 97% | 101% | 95% | 81% | | | |
| SAU100679 | SeqID | 10303 | | 10997 | 11453 | 11713 | 11799 | 12137 | 13329 | 13757 | | | |
| | IDENTITY | 32% | | 31% | 32% | 33% | 35% | 100% | 42% | 35% | | | |
| | COVERAGE | 96% | | 99% | 106% | 96% | 97% | 100% | 104% | 96% | | | |
| SAU100684 | SeqID | 10412 | | | 11486 | | 12097 | 12632 | | 13749 | | | |
| | IDENTITY | 46% | | | 40% | | 46% | 100% | | 46% | | | |
| | COVERAGE | 97% | | | 99% | | 99% | 100% | | 97% | | | |
| SAU100685 | SeqID | | | | | | | 12633 | | | | | |
| | IDENTITY | | | | | | | 100% | | | | | |
| | COVERAGE | | | | | | | 100% | | | | | |
| SAU100689 | SeqID | | 10694 | | | | | 12323 | 13311 | | | | |
| | IDENTITY | | 55% | | | | | 100% | 46% | | | | |
| | COVERAGE | | 98% | | | | | 100% | 96% | | | | |
| SAU100702 | SeqID | | 10655 | | | | | 12196 | 13671 | | | | |
| | IDENTITY | | 46% | | | | | 100% | 41% | | | | |
| | COVERAGE | | 97% | | | | | 100% | 91% | | | | |
| SAU100710 | SeqID | | | | | | 11908 | 12546 | | | | | |
| | IDENTITY | | | | | | 27% | 100% | | | | | |
| | COVERAGE | | | | | | 73% | 101% | | | | | |
| SAU100714 | SeqID | 10465 | 10675 | 11238 | 11563 | | 11961 | 12635 | 13382 | 13853 | | | |
| | IDENTITY | 48% | 66% | 41% | 41% | | 44% | 100% | 60% | 48% | | | |
| | COVERAGE | 108% | 100% | 110% | 102% | | 108% | 103% | 101% | 108% | | | |
| SAU100731 | SeqID | 10071 | 10688 | 11019 | 11371 | | 11850 | 12601 | 13319 | 13945 | | | |
| | IDENTITY | 62% | 79% | 67% | 40% | | 63% | 100% | 76% | 60% | | | |
| | COVERAGE | 99% | 100% | 100% | | 101% | 99% | 101% | 100% | 101% | | | |
| SAU100733 | SeqID | 10415 | | | 11611 | 11636 | 12084 | 12602 | | 13746 | | | |
| | IDENTITY | 41% | | | 33% | 42% | 42% | 100% | | 39% | | | |
| | COVERAGE | 95% | | | 92% | 74% | 95% | 100% | | 95% | | | |
| SAU100734 | SeqID | 10321 | 10573 | 11142 | 11306 | | 12031 | 12603 | 13273 | 13734 | | | |
| | IDENTITY | 28% | 36% | 29% | 27% | | 28% | 100% | 31% | 29% | | | |
| | COVERAGE | 98% | 95% | 97% | 90% | | 93% | 100% | 72% | 101% | | | |
| SAU100736 | SeqID | | 10585 | | | | | 12391 | 13404 | | | | |
| | IDENTITY | | 27% | | | | | 100% | 26% | | | | |
| | COVERAGE | | 97% | | | | | 100% | 97% | | | | |
| SAU100738 | SeqID | 10188 | 10847 | 10953 | 11600 | 11634 | 11907 | 12624 | 13169 | 13981 | | | |
| | IDENTITY | 48% | 45% | 46% | 42% | 48% | 51% | 100% | 45% | 49% | | | |
| | COVERAGE | 97% | 98% | 98% | 97% | 94% | 97% | 100% | 97% | 97% | | | |
| SAU100741 | SeqID | 10081 | 10591 | | 11459 | | 11776 | 12409 | | 13714 | | | |
| | IDENTITY | 65% | 50% | | 35% | | 54% | 100% | | 66% | | | |
| | COVERAGE | 100% | 101% | | 82% | | 100% | 101% | | 101% | | | |
| SAU100745 | SeqID | 10442 | 10484 | 11202 | 11607 | 11733 | 11906 | 12596 | 13453 | 13847 | | | |
| | IDENTITY | 34% | 53% | 35% | 31% | 35% | 34% | 100% | 49% | 35% | | | |
| | COVERAGE | 98% | 97% | 100% | 99% | 101% | 98% | 100% | 98% | 101% | | | |
| SAU100747 | SeqID | | 10749 | | | | | 12597 | 13266 | | | | |
| | IDENTITY | | 32% | | | | | 100% | 31% | | | | |
| | COVERAGE | | 74% | | | | | 100% | 73% | | | | |
| SAU100751 | SeqID | 10425 | 10866 | 11080 | | 11747 | 11927 | 12335 | 13431 | 13788 | | | |
| | IDENTITY | 62% | 64% | 59% | | 62% | 62% | 100% | 63% | 61% | | | |
| | COVERAGE | 99% | 99% | 98% | | 87% | 99% | 100% | 99% | 99% | | | |
| SAU100752 | SeqID | 10140 | | | | | 11976 | 12524 | | 14022 | | | |
| | IDENTITY | 31% | | | | | 35% | 100% | | 38% | | | |
| | COVERAGE | 71% | | | | | 82% | 100% | 72% | | | | |
| SAU100767 | SeqID | 10290 | | | | | 12094 | 12579 | | 13875 | | | |
| | IDENTITY | 43% | | | | | 42% | 100% | | 42% | | | |
| | COVERAGE | 100% | | | | | 90% | 100% | | 100% | | | |
| SAU100771 | SeqID | 10084 | | | | | 11821 | 12545 | 13306 | 13710 | | | |
| | IDENTITY | 30% | | | | | 29% | 100% | 28% | 26% | | | |
| | COVERAGE | 88% | | | | | 80% | 101% | 90% | 94% | | | |
| SAU100773 | SeqID | 10055 | 10758 | 11093 | 11336 | 11763 | 11928 | 12377 | 13250 | | | | |
| | IDENTITY | 47% | 70% | 41% | 41% | 46% | 51% | 100% | 70% | | | | |
| | COVERAGE | 94% | 100% | 98% | 96% | 94% | 93% | 101% | 96% | | | | |
| SAU100776 | SeqID | | | | | | | 12482 | | | | | |
| | IDENTITY | | | | | | | 100% | | | | | |
| | COVERAGE | | | | | | | 100% | | | | | |
| SAU100778 | SeqID | 10083 | | 10957 | | | 11970 | 12514 | | 14062 | | | |
| | IDENTITY | 52% | | 52% | | | 45% | 100% | | 47% | | | |
| | COVERAGE | 89% | | 89% | | | 88% | 100% | | 89% | | | |
| SAU100793 | SeqID | | | | | | | 12188 | 13392 | | | | |
| | IDENTITY | | | | | | | 100% | 27% | | | | |
| | COVERAGE | | | | | | | 100% | 103% | | | | |
| SAU100794 | SeqID | 10203 | | | | | | 12189 | | | | | |
| | IDENTITY | 25% | | | | | | 100% | | | | | |
| | COVERAGE | 101% | | | | | 100% | | | | | | |
| SAU100799 | SeqID | | | | | | | 12682 | | | | | |
| | IDENTITY | | | | | | | 100% | | | | | |
| | COVERAGE | | | | | | | 100% | | | | | |
| SAU100808 | SeqID | | | | | | | 12345 | | 14081 | | | |
| | IDENTITY | | | | | | | 100% | | 35% | | | |
| | COVERAGE | | | | | | | 100% | | 70% | | | |
| SAU100810 | SeqID | 10070 | | | | | 11824 | 12343 | | 14080 | | | |
| | IDENTITY | 51% | | | | | 49% | 100% | | 50% | | | |
| | COVERAGE | 94% | | | | | 96% | 100% | | 96% | | | |
| SAU100813 | SeqID | 10314 | 10764 | 11216 | 11501 | | 5198 | 12322 | 13381 | 13765 | | | |
| | IDENTITY | 47% | 63% | 47% | 45% | | 48% | 100% | 58% | 50% | | | |
| | COVERAGE | 98% | 94% | 100% | 91% | | 92% | 100% | 95% | 92% | | | |
| SAU100831 | SeqID | 10376 | 10741 | 11058 | | | 12093 | 12403 | 13349 | 13811 | | | |
| | IDENTITY | 42% | 58% | 42% | | | 42% | 100% | 51% | 42% | | | |
| | COVERAGE | 97% | 98% | 102% | | | 98% | 100% | 98% | 101% | | | |
| SAU100836 | SeqID | | | | | | | 12212 | | | | | |
| | IDENTITY | | | | | | | 100% | | | | | |
| | COVERAGE | | | | | | | 100% | | | | | |
| SAU100838 | SeqID | | | | | | | 12211 | | | | | |
| | IDENTITY | | | | | | | 100% | | | | | |
| | COVERAGE | | | | | | | 100% | | | | | |
| SAU100839 | SeqID | | 10794 | | | | | 12210 | 13183 | | | | |
| | IDENTITY | | 42% | | | | | 100% | 44% | | | | |
| | COVERAGE | | 100% | | | | 100% | 100% | | | | | |
| SAU100843 | SeqID | 10126 | 10921 | 10974 | 11342 | | | 12328 | 13601 | 14092 | | | |
| | IDENTITY | 26% | 28% | 28% | 28% | | | 100% | 26% | 26% | | | |
| | COVERAGE | 101% | 73% | 101% | 102% | | | 100% | 100% | 104% | | | |
| SAU100845 | SeqID | | | | | | | 12329 | | | | | |
| | IDENTITY | | | | | | | 100% | | | | | |
| | COVERAGE | | | | | | | 100% | | | | | |
| SAU100858 | SeqID | 10256 | 10776 | | 11367 | 11719 | | 12401 | 13472 | 13796 | | | |
| | IDENTITY | 37% | 48% | | 35% | 37% | | 100% | 39% | 39% | | | |
| | COVERAGE | 106% | 98% | | 103% | 106% | | 101% | 100% | 106% | | | |
| SAU100859 | SeqID | 10446 | 10777 | 11254 | 11548 | | 12071 | 12402 | 13473 | 14026 | | | |
| | IDENTITY | 33% | 38% | 33% | 35% | | 34% | 100% | 38% | 32% | | | |
| | COVERAGE | 94% | 94% | 95% | 96% | | 94% | 100% | 92% | 95% | | | |
| SAU100865 | SeqID | 10252 | 10877 | 11010 | 11406 | | 11956 | 12648 | 13506 | 13704 | | | |
| | IDENTITY | 39% | 49% | 41% | 28% | | 44% | 100% | 48% | 38% | | | |
| | COVERAGE | 100% | 99% | 100% | 101% | 99% | 100% | 99% | 100% | | | | |
| SAU100866 | SeqID | 10191 | 10878 | 11005 | 11347 | | 11815 | 12553 | 13492 | 13754 | | | |
| | IDENTITY | 54% | 64% | 51% | 51% | | 53% | 100% | 57% | 55% | | | |
| | COVERAGE | 100% | 100% | | 100% | 100% | 100% | 100% | | | | | |
| SAU100879 | SeqID | | | | | | | 12483 | | | | | |
| | IDENTITY | | | | | | | 100% | | | | | |
| | COVERAGE | | | | | | | 100% | | | | | |
| SAU100880 | SeqID | 10429 | 10720 | | 11335 | | 12039 | 12340 | 13451 | 14072 | | | |
| | IDENTITY | 31% | 51% | | 35% | | 36% | 100% | 45% | 32% | | | |
| | COVERAGE | 81% | 95% | | 97% | | 81% | 100% | 99% | 85% | | | |
| SAU100882 | SeqID | 10322 | 10750 | 11177 | 11351 | | 12018 | 12374 | 13330 | 13733 | | | |
| | IDENTITY | 43% | 54% | 42% | 40% | | 45% | 100% | 52% | 43% | | | |
| | COVERAGE | 98% | 98% | 98% | 99% | | 98% | 100% | 98% | 98% | | | |
| SAU100885 | SeqID | 10410 | 10754 | 11001 | 11509 | | 12095 | 12376 | 14032 | | | | |
| | IDENTITY | 52% | 67% | 53% | 52% | | 53% | 100% | 52% | | | | |
| | COVERAGE | 93% | 74% | 94% | 96% | | 92% | 100% | 93% | | | | |
| SAU100886 | SeqID | 10224 | 10701 | 11213 | 11357 | | 11905 | 12139 | 13348 | 13957 | | | |
| | IDENTITY | 38% | 60% | 38% | 36% | | 36% | 100% | 52% | 38% | | | |
| | COVERAGE | 97% | 83% | 93% | 99% | | 104% | 100% | 102% | 98% | | | |
| SAU100887 | SeqID | 10393 | 10952 | 11057 | 11330 | | 11774 | 12138 | 13342 | 13920 | | | |
| | IDENTITY | 50% | 51% | 50% | 49% | | 48% | 100% | 70% | 50% | | | |
| | COVERAGE | 85% | 96% | 82% | 83% | | 83% | 100% | 96% | 85% | | | |
| SAU100899 | SeqID | | | | | | | 12277 | | | | | |
| | IDENTITY | | | | | | | 100% | | | | | |
| | COVERAGE | | | | | | | 100% | | | | | |
| SAU100901 | SeqID | | | | | | | 12278 | | | | | |
| | IDENTITY | | | | | | | 100% | | | | | |
| | COVERAGE | | | | | | | 100% | | | | | |
| SAU100916 | SeqID | 10209 | 10887 | | | | | 12394 | | 13876 | | | |
| | IDENTITY | 32% | 34% | | | | | 100% | | 32% | | | |
| | COVERAGE | 75% | 72% | | | | | 101% | | 75% | | | |
| SAU100920 | SeqID | 10060 | 10772 | 11191 | 11530 | 11756 | 11983 | 12395 | | 13896 | | | |
| | IDENTITY | 43% | 48% | 31% | 28% | 40% | 30% | 100% | | 43% | | | |
| | COVERAGE | 91% | 86% | 87% | 91% | 86% | 90% | 100% | | 91% | | | |
| SAU100921 | SeqID | 10027 | 10773 | 11185 | | 12012 | 12396 | 13478 | 14074 | | | | |
| | IDENTITY | 32% | 43% | 33% | | 33% | 100% | 34% | 32% | | | | |
| | COVERAGE | 101% | 96% | 96% | | | 96% | 100% | 98% | 101% | | | |
| SAU100932 | SeqID | 10095 | | 11271 | | | 11834 | 12615 | | 14055 | | | |
| | IDENTITY | 39% | | 36% | | | 39% | 100% | | 39% | | | |
| | COVERAGE | 101% | | 101% | | | 102% | 100% | 101% | | | | |
| SAU100944 | SeqID | 10017 | 10687 | 11219 | 11506 | | 12057 | 12505 | 13498 | 14012 | | | |
| | IDENTITY | 37% | 26% | 36% | 36% | | 39% | 100% | 27% | 39% | | | |
| | COVERAGE | 80% | 108% | 79% | 79% | | 83% | 100% | 83% | 80% | | | |
| SAU100952 | SeqID | | 10717 | | | | | 12523 | 13312 | | | | |
| | IDENTITY | | 33% | | | | | 100% | 31% | | | | |
| | COVERAGE | | 104% | | | | | 100% | 102% | | | | |
| SAU100959 | SeqID | | 10704 | | | | | 12485 | 13504 | | | | |
| | IDENTITY | | 58% | | | | | 100% | 49% | | | | |
| | COVERAGE | | 99% | | | | | 100% | 101% | | | | |
| SAU100961 | SeqID | 10320 | 10671 | 11141 | 11312 | | 12030 | 12638 | 13322 | 13766 | | | |
| | IDENTITY | 42% | 63% | 47% | 40% | | 50% | 100% | 57% | 42% | | | |
| | COVERAGE | 98% | 99% | 98% | 97% | | 98% | 101% | 101% | 99% | | | |
| SAU100962 | SeqID | | 11299 | | | 12639 | 13577 | | | | | | |
| | IDENTITY | | 28% | | | 100% | 26% | | | | | | |
| | COVERAGE | | 80% | | | 101% | 92% | | | | | | |
| SAU100963 | SeqID | 10319 | 10633 | 11140 | 11493 | | 12029 | 12640 | 13320 | 13771 | | | |
| | IDENTITY | 60% | 79% | 59% | 61% | | 63% | 100% | 81% | 60% | | | |
| | COVERAGE | 84% | 96% | 81% | 81% | | 84% | 101% | 92% | 88% | | | |
| SAU100964 | SeqID | | 10501 | 11139 | | | 12028 | 12641 | 13331 | | | | |
| | IDENTITY | | 61% | 45% | | | 47% | 100% | 60% | | | | |
| | COVERAGE | | 101% | 76% | | | 77% | 100% | 101% | | | | |
| SAU100965 | SeqID | | | | | | | 12642 | | | | | |
| | IDENTITY | | | | | | | 100% | | | | | |
| | COVERAGE | | | | | | | 101% | | | | | |
| SAU100970 | SeqID | 10128 | 10516 | 11247 | 11512 | | 11891 | 12529 | 13362 | | | | |
| | IDENTITY | 52% | 54% | 39% | 47% | | 52% | 100% | 46% | | | | |
| | COVERAGE | 99% | 99% | 100% | 100% | 99% | 100% | 99% | | | | | |
| SAU100996 | SeqID | | 10686 | | 11350 | | | 12606 | 13600 | | | | |
| | IDENTITY | | 38% | | 34% | | | 100% | 39% | | | | |
| | COVERAGE | | 97% | | 73% | | | 100% | 96% | | | | |
| SAU101006 | SeqID | 10185 | 10572 | 11022 | 11473 | | 5122 | 12190 | | 13820 | | | |
| | IDENTITY | 29% | 40% | 31% | 26% | | 26% | 100% | | 30% | | | |
| | COVERAGE | 84% | 98% | 87% | 94% | | 79% | 100% | 91% | | | | |
| SAU101020 | SeqID | | | | | | | 12710 | | | | | |
| | IDENTITY | | | | | | | 100% | | | | | |
| | COVERAGE | | | | | | | 100% | | | | | |
| SAU101024 | SeqID | | | | | | | 12711 | | | | | |
| | IDENTITY | | | | | | | 100% | | | | | |
| | COVERAGE | | | | | | | 101% | | | | | |
| SAU101028 | SeqID | 10034 | 10848 | 11148 | 11364 | | 12006 | 12552 | 13471 | 13901 | | | |
| | IDENTITY | 46% | 57% | 43% | 46% | | 46% | 100% | 55% | 45% | | | |
| | COVERAGE | 106% | 101% | 107% | 100% | | 108% | 100% | 100% | 106% | | | |
| SAU101034 | SeqID | | 10578 | | | | | 12608 | 13654 | | | | |
| | IDENTITY | | 36% | | | | | 100% | 37% | | | | |
| | COVERAGE | | 80% | | | | | 100% | 71% | | | | |
| SAU101038 | SeqID | | 10716 | | | | 11822 | 12521 | 13428 | | | | |
| | IDENTITY | | 42% | | | | 35% | 100% | 36% | | | | |
| | COVERAGE | | 96% | | | | 78% | 101% | 103% | | | | |
| SAU101039 | SeqID | | | | | | | 12522 | | | | | |
| | IDENTITY | | | | | | | 100% | | | | | |
| | COVERAGE | | | | | | | 100% | | | | | |
| SAU101065 | SeqID | 10221 | 10681 | 11210 | 11607 | 11668 | 11801 | 12289 | 13191 | 14027 | | | |
| | IDENTITY | 37% | 49% | 40% | 28% | 38% | 36% | 100% | 46% | 31% | | | |
| | COVERAGE | 98% | 103% | 100% | 108% | 97% | 98% | 100% | 102% | 98% | | | |
| SAU101067 | SeqID | | 10682 | | | | | 12290 | 13394 | | | | |
| | IDENTITY | | 41% | | | | | 100% | 40% | | | | |
| | COVERAGE | | 100% | | | | | 100% | 99% | | | | |
| SAU101070 | SeqID | | 10770 | | | | | 12291 | 13380 | | | | |
| | IDENTITY | | 40% | | | | | 100% | 32% | | | | |
| | COVERAGE | | 89% | | | | | 100% | 82% | | | | |
| SAU101084 | SeqID | 10066 | | 11156 | | | 11974 | 12283 | | | | | |
| | IDENTITY | 36% | | 34% | | | 35% | 100% | | | | | |
| | COVERAGE | 90% | | 102% | | | 92% | 100% | | | | | |
| SAU101085 | SeqID | 10170 | | 11263 | 11462 | | 11973 | 12284 | 13225 | 13993 | | | |
| | IDENTITY | 37% | | 34% | 37% | | 38% | 100% | 47% | 32% | | | |
| | COVERAGE | 89% | | 88% | 94% | | 94% | 100% | 101% | 88% | | | |
| SAU101086 | SeqID | | | | 11366 | | 11972 | 12285 | 13666 | | | | |
| | IDENTITY | | | | 42% | | 34% | 100% | 49% | | | | |
| | COVERAGE | | | | 74% | | 94% | 100% | 101% | | | | |
| SAU101090 | SeqID | | 10755 | | | | | 12191 | 13188 | | | | |
| | IDENTITY | | 36% | | | | | 100% | 31% | | | | |
| | COVERAGE | | 97% | | | | | 100% | 97% | | | | |
| SAU101092 | SeqID | 10450 | 10567 | | | | 11847 | 12192 | | | | | |
| | IDENTITY | 35% | 33% | | | | 30% | 100% | | | | | |
| | COVERAGE | 71% | 96% | | | | 72% | 100% | | | | | |
| SAU101104 | SeqID | 10135 | 10768 | 11248 | 11404 | 11732 | 11869 | 12195 | 13482 | 13827 | | | |
| | IDENTITY | 38% | 45% | 39% | 37% | 37% | 42% | 100% | 38% | 37% | | | |
| | COVERAGE | 98% | 100% | 100% | 92% | 99% | 99% | 100% | 96% | 99% | | | |
| SAU101143 | SeqID | 10040 | | 11157 | 11315 | | 11968 | 12502 | | 13906 | | | |
| | IDENTITY | 47% | | 27% | 43% | | 44% | 100% | | 47% | | | |
| | COVERAGE | 99% | | 82% | 98% | | 100% | 100% | | 99% | | | |
| SAU101145 | SeqID | | 10548 | | | | 12070 | 12299 | | | | | |
| | IDENTITY | | 42% | | | | 43% | 100% | | | | | |
| | COVERAGE | | 98% | | | | 96% | 101% | | | | | |
| SAU101155 | SeqID | 10287 | 10697 | 11077 | 11352 | 11690 | 11944 | 12310 | 13549 | 13868 | | | |
| | IDENTITY | 43% | 49% | 40% | 30% | 42% | 42% | 100% | 37% | 43% | | | |
| | COVERAGE | 95% | 95% | 95% | 86% | 95% | 94% | 100% | 76% | 95% | | | |
| SAU101156 | SeqID | 10426 | 10698 | 11032 | 11333 | | 12083 | 12311 | | 13790 | | | |
| | IDENTITY | 56% | 63% | 60% | 52% | | 58% | 100% | | 55% | | | |
| | COVERAGE | 96% | 101% | 96% | 97% | | 96% | 101% | | 96% | | | |
| SAU101159 | SeqID | | 10891 | | 11532 | | | 12331 | 13463 | | | | |
| | IDENTITY | | 65% | | 36% | | | 100% | 54% | | | | |
| | COVERAGE | | 100% | | 100% | | | 100% | 104% | | | | |
| SAU101175 | SeqID | | | | | | | 12213 | | | | | |
| | IDENTITY | | | | | | | 100% | | | | | |
| | COVERAGE | | | | | | | 101% | | | | | |
| SAU101180 | SeqID | 10061 | 10888 | | | | 11910 | 12656 | | | | | |
| | IDENTITY | 38% | 50% | | | | 37% | 100% | | | | | |
| | COVERAGE | 72% | 89% | | | | 70% | 100% | | | | | |
| SAU101183 | SeqID | | 10843 | | | | | 12304 | | | | | |
| | IDENTITY | | 42% | | | | | 100% | | | | | |
| | COVERAGE | | 102% | | | | 100% | | | | | | |
| SAU101184 | SeqID | 10477 | 10711 | 11218 | 11376 | 11735 | 12033 | 12305 | 13499 | 13709 | | | |
| | IDENTITY | 37% | 46% | 36% | 30% | 38% | 35% | 100% | 44% | 38% | | | |
| | COVERAGE | 86% | 100% | 102% | 85% | 82% | 85% | 100% | 98% | 82% | | | |
| SAU101189 | SeqID | | | | | | | 12264 | | | | | |
| | IDENTITY | | | | | | | 100% | | | | | |
| | COVERAGE | | | | | | | 100% | | | | | |
| SAU101197 | SeqID | 10180 | 10787 | 11024 | | | 11924 | 12300 | 13340 | 13976 | | | |
| | IDENTITY | 31% | 44% | 31% | | | 27% | 100% | 46% | 30% | | | |
| | COVERAGE | 98% | 98% | 101% | | | 100% | 100% | 98% | 98% | | | |
| SAU101198 | SeqID | 10218 | 10786 | 11023 | | | 11923 | 12301 | 13341 | | | | |
| | IDENTITY | 43% | 50% | 43% | | | 41% | 100% | 46% | | | | |
| | COVERAGE | 74% | 98% | 73% | | | 75% | 100% | 102% | | | | |
| SAU101199 | SeqID | 10088 | 10742 | 10970 | | | 11949 | 12302 | 13178 | 14052 | | | |
| | IDENTITY | 29% | 40% | 31% | | | 36% | 100% | 37% | 30% | | | |
| | COVERAGE | 97% | 86% | 94% | | | 97% | 100% | 87% | 98% | | | |
| SAU101220 | SeqID | 10286 | 10864 | | | | | 12645 | 13390 | 13870 | | | |
| | IDENTITY | 32% | 37% | | | | | 100% | 39% | 31% | | | |
| | COVERAGE | 74% | 81% | | | | | 100% | 99% | 74% | | | |
| SAU101224 | SeqID | | | | 11533 | | | 12647 | | | | | |
| | IDENTITY | | | | 28% | | | 100% | | | | | |
| | COVERAGE | | | | 77% | | | 100% | | | | | |
| SAU101226 | SeqID | | 10837 | | | 11658 | 11825 | 12298 | 13296 | 13721 | | | |
| | IDENTITY | | 52% | | | 28% | 37% | 100% | 27% | 27% | | | |
| | COVERAGE | | 96% | | | 75% | 90% | 100% | 77% | 77% | | | |
| SAU101231 | SeqID | 10301 | 10513 | | | | 12079 | 12303 | | 13759 | | | |
| | IDENTITY | 32% | 61% | | | | 32% | 100% | | 31% | | | |
| | COVERAGE | 101% | 100% | | | | 73% | 101% | | 106% | | | |
| SAU101235 | SeqID | | 10616 | 11087 | | | | 12561 | 13486 | | | | |
| | IDENTITY | | 37% | 27% | | | | 100% | | 35% | | | |
| | COVERAGE | | 84% | 90% | | | | 100% | | 97% | | | |
| SAU101236 | SeqID | 10089 | 10500 | | | 11673 | 11951 | 12564 | 13474 | | | | |
| | IDENTITY | 42% | 55% | | | 29% | 39% | 100% | | 35% | | | |
| | COVERAGE | 101% | 77% | | | 108% | 100% | 100% | 103% | | | | |
| SAU101239 | SeqID | | | | 11361 | | | 12570 | | | | | |
| | IDENTITY | | | | 33% | | | 100% | | | | | |
| | COVERAGE | | | | 98% | | | 100% | | | | | |
| SAU101240 | SeqID | | | | | | | 12573 | | | | | |
| | IDENTITY | | | | | | | 100% | | | | | |
| | COVERAGE | | | | | | | 101% | | | | | |
| SAU101242 | SeqID | 10335 | 10879 | 11121 | 11425 | | 11988 | 12578 | 13240 | 14095 | | | |
| | IDENTITY | 48% | 67% | 47% | 48% | | 47% | 100% | 55% | 47% | | | |
| | COVERAGE | 104% | 101% | 104% | 105% | | 104% | 101% | 101% | 105% | | | |
| SAU101247 | SeqID | | 10919 | | | | 11984 | 12512 | 13359 | | | | |
| | IDENTITY | | 32% | | | | 36% | 100% | | 33% | | | |
| | COVERAGE | | 91% | | | | 90% | 100% | 85% | | | | |
| SAU101262 | SeqID | 1013710735 | | 11399 | | 11922 | 12488 | 13238 | 13837 | | | | |
| | IDENTITY | 28% | 70% | | 47% | | 33% | 100% | 67% | 28% | | | |
| | COVERAGE | 73% | 100% | | 101% | | 97% | 100% | 100% | 73% | | | |
| SAU101266 | SeqID | 10238 | 10789 | 11178 | 11517 | | 11829 | 12490 | 13317 | 13864 | | | |
| | IDENTITY | 45% | 57% | 46% | 41% | | 43% | 100% | 51% | 44% | | | |
| | COVERAGE | 100% | 99% | 100% | 98% | | 89% | 100% | 98% | 100% | | | |
| SAU101267 | SeqID | | | | | | | 12364 | | | | | |
| | IDENTITY | | | | | | | 100% | | | | | |
| | COVERAGE | | | | | | | 100% | | | | | |
| SAU101270 | SeqID | 10175 | 10718 | 11220 | 11324 | | 11881 | 12365 | 13383 | 13942 | | | |
| | IDENTITY | 50% | 62% | 47% | 45% | | 52% | 100% | 61% | 50% | | | |
| | COVERAGE | 96% | 99% | 97% | 93% | | 97% | 100% | 98% | 96% | | | |
| SAU101271 | SeqID | 10174 | 10719 | 11221 | 11556 | | 11880 | 12366 | 13385 | 13943 | | | |
| | IDENTITY | 37% | 46% | 36% | 25% | | 35% | 100% | 46% | 37% | | | |
| | COVERAGE | 100% | 102% | 100% | 100% | | 100% | 100% | 101% | 75% | | | |
| SAU101275 | SeqID | 10232 | 10684 | 10981 | 11521 | 11708 | 11845 | 12604 | 13299 | 13954 | | | |
| | IDENTITY | 35% | 57% | 38% | 33% | 34% | 34% | 100% | 57% | 35% | | | |
| | COVERAGE | 95% | 101% | 93% | 98% | 96% | 94% | 100% | 101% | 95% | | | |
| SAU101286 | SeqID | | 10884 | | | | | 12292 | 13189 | | | | |
| | IDENTITY | | 47% | | | | | 100% | 40% | | | | |
| | COVERAGE | | 100% | | | | | 101% | 99% | | | | |
| SAU101293 | SeqID | | | | | | | 12631 | | | | | |
| | IDENTITY | | | | | | | 100% | | | | | |
| | COVERAGE | | | | | | | 101% | | | | | |
| SAU101300 | SeqID | | 10751 | | | | | 12557 | 13194 | | | | |
| | IDENTITY | | 57% | | | | | 100% | 54% | | | | |
| | COVERAGE | | 93% | | | | | 101% | 90% | | | | |
| SAU101301 | SeqID | | 10752 | | | | 11785 | 12558 | 13195 | | | | |
| | IDENTITY | | 57% | | | | 27% | 100% | 54% | | | | |
| | COVERAGE | | 96% | | | | 94% | 101% | 99% | | | | |
| SAU101302 | SeqID | | 10753 | | 11317 | | | 12559 | 13611 | | | | |
| | IDENTITY | | 49% | | 33% | | | 100% | 26% | | | | |
| | COVERAGE | | 101% | | 86% | | | 101% | 72% | | | | |
| SAU101310 | SeqID | 10330 | 10924 | 11160 | 11321 | | 12063 | 12562 | 13364 | 13885 | | | |
| | IDENTITY | 47% | 52% | 48% | 43% | | 47% | 100% | 51% | 47% | | | |
| | COVERAGE | 98% | 98% | 98% | 98% | | 98% | 100% | 98% | 98% | | | |
| SAU101311 | SeqID | 10094 | | 11278 | | 11859 | 12563 | | 13891 | | | | |
| | IDENTITY | 46% | | 46% | | 42% | 100% | | 46% | | | | |
| | COVERAGE | 98% | | 98% | | 96% | 100% | | 95% | | | | |
| SAU101320 | SeqID | 10263 | 10861 | 10965 | 11562 | | 11948 | 12128 | 13254 | 14089 | | | |
| | IDENTITY | 50% | 59% | 49% | 39% | | 51% | 100% | 56% | 49% | | | |
| | COVERAGE | 100% | 99% | 99% | 100% | | 99% | 100% | 97% | 100% | | | |
| SAU101327 | SeqID | 10018 | 10710 | 11147 | | | 11779 | 12612 | 13495 | 14014 | | | |
| | IDENTITY | 35% | 46% | 43% | | | 34% | 100% | 35% | 35% | | | |
| | COVERAGE | 100% | 97% | 101% | | | 92% | 101% | 99% | 100% | | | |
| SAU101339 | SeqID | 10093 | 10520 | | 11365 | | 11839 | 12399 | 13405 | 13888 | | | |
| | IDENTITY | 55% | 30% | | 26% | | 54% | 100% | 27% | 45% | | | |
| | COVERAGE | 99%74% | | 74% | | 97% | 100% | 76% | 99% | | | | |
| SAU101340 | SeqID | 10092 | | | | | 11840 | 12400 | | 13889 | | | |
| | IDENTITY | 37% | | | | | 35% | 100% | | 39% | | | |
| | COVERAGE | 106% | | | | | 101% | 101% | | 104% | | | |
| SAU101341 | SeqID | 10230 | 10925 | 11212 | 11385 | | 11898 | 12618 | 13365 | 13952 | | | |
| | IDENTITY | 47% | 55% | 48% | 48% | | 45% | 100% | 48% | 47% | | | |
| | COVERAGE | 93% | 92% | 92% | 98% | | 92% | 100% | 100% | 93% | | | |
| SAU101343 | SeqID | 10422 | 10649 | 11162 | | 11721 | | 12619 | 13346 | 13785 | | | |
| | IDENTITY | 50% | 55% | 49% | | 50% | | 100% | 58% | 51% | | | |
| | COVERAGE | 99% | 100% | 99% | | 99% | | 100% | 92% | 99% | | | |
| SAU101344 | SeqID | 10171 | 10650 | 11252 | | | 11826 | 12620 | 13347 | 13755 | | | |
| | IDENTITY | 48% | 62% | 40% | | | 37% | 100% | 44% | 38% | | | |
| | COVERAGE | 81% | 88% | 79% | | | 82% | 100% | 79% | 81% | | | |
| SAU101346 | SeqID | 10058 | | | 11282 | | 11803 | 12621 | | 13894 | | | |
| | IDENTITY | 36% | | | 35% | | 43% | 100% | | 36% | | | |
| | COVERAGE | 99% | | | 103% | | 99% | 100% | | 99% | | | |
| SAU101347 | SeqID | 10139 | | 11163 | 11283 | | 11877 | 12622 | 13259 | 13839 | | | |
| | IDENTITY | 63% | | 29% | 62% | | 62% | 100% | 30% | 62% | | | |
| | COVERAGE | 100% | | 96% | 101% | | 100% | 100% | 91% | 100% | | | |
| SAU101350 | SeqID | 10184 | 10508 | | 11318 | | 12069 | 12487 | 13286 | 13982 | | | |
| | IDENTITY | 61% | 56% | | 32% | | 46% | 100% | 55% | 60% | | | |
| | COVERAGE | 95% | 98% | | 81% | | 100% | 100% | 97% | 97% | | | |
| SAU101351 | SeqID | | 10507 | | | | | 12486 | 13285 | | | | |
| | IDENTITY | | 60% | | | | | 100% | 59% | | | | |
| | COVERAGE | | 96% | | | | | 100% | 96% | | | | |
| SAU101360 | SeqID | 10138 | 10571 | 10977 | 11598 | 11684 | 11878 | 12555 | 13175 | 13838 | | | |
| | IDENTITY | 56% | 70% | 54% | 35% | 55% | 58% | 100% | 71% | 56% | | | |
| | COVERAGE | 98% | 101% | 98% | 97% | 88% | 98% | 100% | 101% | 98% | | | |
| SAU101365 | SeqID | 10269 | 10491 | 11127 | 11577 | 11809 | 12556 | 13295 | 13874 | | | | |
| | IDENTITY | 45% | 55% | 44% | 40% | 45% | 100% | 50% | 45% | | | | |
| | COVERAGE | 101% | 101% | 101% | 99% | 101% | 100% | 100% | 101% | | | | |
| SAU101366 | SeqID | 10147 | 10654 | | | | | 12266 | 13179 | 13843 | | | |
| | IDENTITY | 49% | 73% | | | | | 100% | 56% | 48% | | | |
| | COVERAGE | 99% | 98% | | | | | 100% | 99% | 99% | | | |
| SAU101369 | SeqID | | | | | | | 12274 | | | | | |
| | IDENTITY | | | | | | | 100% | | | | | |
| | COVERAGE | | | | | | | 100% | | | | | |
| SAU101371 | SeqID | | | | 11372 | | 11902 | 12275 | 13243 | | | | |
| | IDENTITY | | | | 40% | | 32% | 100% | 34% | | | | |
| | COVERAGE | | | | 86% | | 79% | 100% | 77% | | | | |
| SAU101381 | SeqID | 10373 | | | | | | 12145 | 13432 | | | | |
| | IDENTITY | 26% | | | | | | 100% | 41% | | | | |
| | COVERAGE | 98% | | | | | | 100% | 99% | | | | |
| SAU101382 | SeqID | 10239 | 10707 | 11179 | 11292 | 11635 | 11879 | 12146 | 13657 | 13862 | | | |
| | IDENTITY | 53% | 60% | 50% | 42% | 39% | 53% | 100% | 63% | 52% | | | |
| | COVERAGE | 98% | 99% | 97% | 97% | 79% | 98% | 100% | 96% | 98% | | | |
| SAU101383 | SeqID | 10317 | 10625 | 11226 | 11418 | | 12055 | 12147 | 13422 | 13761 | | | |
| | IDENTITY | 37% | 39% | 36% | 26% | | 38% | 100% | 37% | 39% | | | |
| | COVERAGE | 102% | 90% | 97% | 98% | | 94% | 100% | 112% | 94% | | | |
| SAU101385 | SeqID | 10403 | 10830 | 11030 | 11368 | 11640 | 12115 | 12385 | 13508 | 14067 | | | |
| | IDENTITY | 33% | 52% | 31% | 27% | 32% | 29% | 100% | 38% | 32% | | | |
| | COVERAGE | 99% | 90% | 92% | 89% | 96% | 98% | 100% | 92% | 99% | | | |
| SAU101387 | SeqID | 10402 | 10839 | | 11549 | | 12114 | 12386 | 13509 | 14068 | | | |
| | IDENTITY | 27% | 35% | | 27% | | 27% | 100% | 32% | 27% | | | |
| | COVERAGE | 87% | 88% | | 71% | | 87% | 101% | 90% | 87% | | | |
| SAU101389 | SeqID | 10401 | 10801 | 11029 | 11400 | | 12113 | 12387 | 13510 | 14069 | | | |
| | IDENTITY | 55% | 72% | 57% | 60% | | 57% | 100% | 74% | 55% | | | |
| | COVERAGE | 98% | 99% | 99% | 100% | | 98% | 100% | 94% | 98% | | | |
| SAU101398 | SeqID | 10313 | 10881 | 11224 | 11502 | 11754 | 12051 | 12324 | 13485 | 13767 | | | |
| | IDENTITY | 55% | 78% | 54% | 51% | 57% | 56% | 100% | 68% | 54% | | | |
| | COVERAGE | 100% | 101% | 100% | 99% | 101% | 100% | 101% | 101% | 101% | | | |
| SAU101399 | SeqID | 10312 | 10882 | 10989 | 11416 | 11755 | 12050 | 12325 | 13699 | 13768 | | | |
| | IDENTITY | 50% | 63% | 48% | 38% | 51% | 51% | 100% | 58% | 49% | | | |
| | COVERAGE | 99% | 100% | 98% | 97% | 85% | 97% | 100% | 99% | 99% | | | |
| SAU101400 | SeqID | | 10743 | | 11448 | | | 12326 | 13391 | | | | |
| | IDENTITY | | 46% | | 32% | | | 100% | 41% | | | | |
| | COVERAGE | | 96% | | 95% | | | 100% | 96% | | | | |
| SAU101408 | SeqID | 10267 | 10509 | | | | | 12308 | 13278 | 14050 | | | |
| | IDENTITY | 37% | 43% | | | | | 100% | 42% | 39% | | | |
| | COVERAGE | 100% | 99% | | | | | 100% | 101% | 100% | | | |
| SAU101421 | SeqID | | 10676 | | | | | 12498 | | | | | |
| | IDENTITY | | 38% | | | | | 100% | | | | | |
| | COVERAGE | | 93% | | | | | 100% | | | | | |
| SAU101427 | SeqID | | | | | | | 12500 | 13234 | | | | |
| | IDENTITY | | | | | | | 100% | 48% | | | | |
| | COVERAGE | | | | | | | 100% | 100% | | | | |
| SAU101432 | SeqID | | | 11046 | 11286 | 11744 | 12065 | 12184 | 13538 | | | | |
| | IDENTITY | | | 57% | 60% | 63% | 68% | 100% | 26% | | | | |
| | COVERAGE | | | 99% | 100% | 101% | 99% | 101% | 73% | | | | |
| SAU101436 | SeqID | 10271 | | 11045 | 11285 | | 12067 | 12183 | | 13873 | | | |
| | IDENTITY | 27% | | 62% | 61% | | 59% | 100% | | 27% | | | |
| | COVERAGE | 90% | | 99% | 97% | | 98% | 100% | | 90% | | | |
| SAU101438 | SeqID | 10146 | 10825 | 11042 | | | | 12379 | 13337 | 13842 | | | |
| | IDENTITY | 30% | 29% | 29% | | | | 100% | 27% | 30% | | | |
| | COVERAGE | 88% | 94% | 89% | | | | 100% | 94% | 88% | | | |
| SAU101444 | SeqID | 10254 | 10827 | 11144 | 11301 | | 12034 | 12381 | 13335 | 13792 | | | |
| | IDENTITY | 60% | 66% | 57% | 54% | | 60% | 100% | 61% | 59% | | | |
| | COVERAGE | 100% | 101% | 100% | 100% | | 100% | 100% | 99% | 100% | | | |
| SAU101445 | SeqID | 10248 | 10828 | 11207 | | | 12037 | 12382 | 13408 | 13949 | | | |
| | IDENTITY | 52% | 70% | 52% | | | 54% | 100% | 72% | 51% | | | |
| | COVERAGE | 99% | 100% | 96% | | | 99% | 100% | 100% | 100% | | | |
| SAU101446 | SeqID | 10411 | 10674 | | | | 11903 | 12383 | | 14031 | | | |
| | IDENTITY | 50% | 59% | | | | 33% | 100% | | 50% | | | |
| | COVERAGE | 98% | 100% | | | | 97% | 100% | | 99% | | | |
| SAU101447 | SeqID | | | | | | | 12683 | | | | | |
| | IDENTITY | | | | | | | 100% | | | | | |
| | COVERAGE | | | | | | | 101% | | | | | |
| SAU101452 | SeqID | | | | | | | 12684 | | | | | |
| | IDENTITY | | | | | | | 100% | | | | | |
| | COVERAGE | | | | | | | 100% | | | | | |
| SAU101455 | SeqID | | | | | | | 12686 | | | | | |
| | IDENTITY | | | | | | | 100% | | | | | |
| | COVERAGE | | | | | | | 100% | | | | | |
| SAU101461 | SeqID | | 10705 | | | | 11790 | 12680 | | | | | |
| | IDENTITY | | 54% | | | | 26% | 100% | | | | | |
| | COVERAGE | | 93% | | | | 86% | 101% | | | | | |
| SAU101463 | SeqID | 10268 | 10708 | | | | 11919 | 12679 | 13584 | 14051 | | | |
| | IDENTITY | 29% | 45% | | | | 26% | 100% | 26% | 29% | | | |
| | COVERAGE | 77% | 98% | | | | 91% | 101% | 88% | 77% | | | |
| SAU101476 | SeqID | 10469 | 10905 | 12254 | 13454 | 13905 | | | | | | | |
| | IDENTITY | 38% | 29% | 100% | 25% | 26% | | | | | | | |
| | COVERAGE | 84% | 94% | | | 100% | 95% | 73% | | | | | |
| SAU101481 | SeqID | 10125 | 10920 | 10975 | 11290 | | 11894 | 12130 | 13580 | | | | |
| | IDENTITY | 40% | 39% | 40% | 32% | | 39% | 100% | 41% | | | | |
| | COVERAGE | 93% | 95% | 96% | 93% | | 96% | 100% | 96% | | | | |
| SAU101482 | SeqID | 10126 | 10921 | 10974 | 11342 | 11738 | 11893 | 12123 | 13360 | 14092 | | | |
| | IDENTITY | 55% | 51% | 52% | 44% | 36% | 52% | 100% | 48% | 37% | | | |
| | COVERAGE | 98% | 100% | 98% | 98% | 77% | 98% | 100% | 99% | 101% | | | |
| SAU101483 | SeqID | 10127 | 10918 | 10973 | 11341 | | 11892 | 12124 | 13674 | 13871 | | | |
| | IDENTITY | 65% | 41% | 59% | 58% | | 61% | 100% | 51% | 31% | | | |
| | COVERAGE | 88% | 90% | 90% | 90% | | 87% | 101% | 92% | 94% | | | |
| SAU101488 | SeqID | | 10730 | | | | 11868 | 12164 | 13450 | 13799 | | | |
| | IDENTITY | | 28% | | | | 25% | 100% | 33% | 28% | | | |
| | COVERAGE | | 95% | | | | 74% | 100% | 98% | 73% | | | |
| SAU101491 | SeqID | | 10580 | | | | | 12165 | 13315 | | | | |
| | IDENTITY | | 42% | | | | | 100% | 42% | | | | |
| | COVERAGE | | 104% | | | | | 100% | 95% | | | | |
| SAU101492 | SeqID | 10073 | 10581 | 11020 | 11284 | | 11831 | 12166 | 13323 | 13715 | | | |
| | IDENTITY | 38% | 52% | 37% | 29% | | 37% | 100% | 43% | 38% | | | |
| | COVERAGE | 98% | 101% | 98% | 78% | | 94% | 101% | 85% | 98% | | | |
| SAU101493 | SeqID | 10074 | | 11021 | 11381 | | 11832 | 12167 | 13564 | 13716 | | | |
| | IDENTITY | 42% | | 41% | 30% | | 43% | 100% | 64% | 44% | | | |
| | COVERAGE | 96% | | 97% | 94% | | 98% | 101% | 91% | 96% | | | |
| SAU101495 | SeqID | 1003010805 | 11188 | 11458 | | 5187 | 12360 | 13333 | 14077 | | | | |
| | IDENTITY | 32% | 34% | 36% | 29% | | 33% | 100% | 32% | 32% | | | |
| | COVERAGE | 92% | 92% | 90% | 86% | | 90% | 100% | 94% | 92% | | | |
| SAU101497 | SeqID | | 10806 | | | | | 12361 | | | | | |
| | IDENTITY | | 59% | | | | | 100% | | | | | |
| | COVERAGE | | 100% | | | | | 100% | | | | | |
| SAU101509 | SeqID | 10121 | | | | 11712 | | 12418 | 13249 | | | | |
| | IDENTITY | 34% | | | | 36% | | 100% | 49% | | | | |
| | COVERAGE | 104% | | | | 104% | | 100% | 83% | | | | |
| SAU101526 | SeqID | | 10901 | | | | | 12179 | 13465 | | | | |
| | IDENTITY | | 38% | | | | | 100% | 34% | | | | |
| | COVERAGE | | 88% | | | | | 100% | 89% | | | | |
| SAU101529 | SeqID | | | | | | | 12544 | | | | | |
| | IDENTITY | | | | | | | 100% | | | | | |
| | COVERAGE | | | | | | | 100% | | | | | |
| SAU101541 | SeqID | 10024 | 10631 | 11182 | 11526 | | 12014 | 12344 | 13647 | 14019 | | | |
| | IDENTITY | 41% | 63% | 42% | 38% | | 42% | 100% | 59% | 40% | | | |
| | COVERAGE | 101% | 100% | 101% | 98% | | 101% | 100% | 101% | 100% | | | |
| SAU101543 | SeqID | 10025 | 10634 | 11183 | | | 11867 | 12346 | 13406 | 14091 | | | |
| | IDENTITY | 26% | 33% | 27% | | | 27% | 100% | 32% | 28% | | | |
| | COVERAGE | 78% | 97% | 78% | | | 73% | 100% | 96% | 76% | | | |
| SAU101545 | SeqID | 10029 | 10636 | 11187 | 11329 | | 12010 | 12348 | 13633 | 14076 | | | |
| | IDENTITY | 31% | 50% | 32% | 27% | | 28% | 100% | 47% | 30% | | | |
| | COVERAGE | 98% | 99% | 97% | 83% | | 97% | 100% | 97% | 98% | | | |
| SAU101546 | SeqID | | 10638 | | | | | 12349 | | | | | |
| | IDENTITY | | 27% | | | | | 100% | | | | | |
| | COVERAGE | | 80% | | | | | 100% | | | | | |
| SAU101549 | SeqID | 10443 | 10762 | 11228 | | 11767 | 12049 | 12549 | 13460 | 14030 | | | |
| | IDENTITY | 40% | 38% | 30% | | 38% | 29% | 100% | 39% | 38% | | | |
| | COVERAGE | 70% | 95% | 88% | | 70% | 92% | 102% | 92% | 70% | | | |
| SAU101551 | SeqID | 10172 | 10490 | 11194 | 11360 | | 12019 | 12550 | 13326 | 13939 | | | |
| | IDENTITY | 52% | 77% | 26% | 27% | | 26% | 100% | 76% | 52% | | | |
| | COVERAGE | 97% | 98% | 98% | 89% | | 96% | 100% | 98% | 97% | | | |
| SAU101554 | SeqID | | 10485 | | 11485 | | | 12551 | 13672 | | | | |
| | IDENTITY | | 48% | | 26% | | | 100% | 46% | | | | |
| | COVERAGE | | 83% | | 81% | | | 101% | 91% | | | | |
| SAU101561 | SeqID | 10400 | 10937 | 11073 | 11355 | 11759 | 12112 | 12149 | 13307 | 14064 | | | |
| | IDENTITY | 44% | 57% | 44% | 38% | 42% | 44% | 100% | 49% | 43% | | | |
| | COVERAGE | 99% | 99% | 99% | 100% | 99% | 100% | 100% | 99% | 99% | | | |
| SAU101565 | SeqID | 10134 | 10552 | 11211 | | | 11895 | 12151 | 13448 | 13826 | | | |
| | IDENTITY | 37% | 50% | 35% | | | 36% | 100% | 44% | 36% | | | |
| | COVERAGE | 93% | 96% | 94% | | | 92% | 100% | 99% | 92% | | | |
| SAU101567 | SeqID | | | | | | | 12144 | | | | | |
| | IDENTITY | | | | | | | 100% | | | | | |
| | COVERAGE | | | | | | | 100% | | | | | |
| SAU101570 | SeqID | 10037 | 10690 | 11208 | | 11700 | 11835 | 12584 | 13563 | 13900 | | | |
| | IDENTITY | 32% | 48% | 31% | | 34% | 33% | 100% | 37% | 30% | | | |
| | COVERAGE | 100% | 100% | 99% | | 95% | 102% | 100% | 100% | 100% | | | |
| SAU101571 | SeqID | | 10691 | | | | 11917 | 12585 | 13308 | | | | |
| | IDENTITY | | 45% | | | | 33% | 100% | 31% | | | | |
| | COVERAGE | | 98% | | | | 94% | 100% | 97% | | | | |
| SAU101572 | SeqID | 10068 | 10692 | | | 11689 | 11864 | 12586 | 13309 | 14083 | | | |
| | IDENTITY | 26% | 56% | | | 46% | 43% | 100% | 45% | 25% | | | |
| | COVERAGE | 75% | 101% | | | 89% | 96% | 100% | 98% | 75% | | | |
| SAU101573 | SeqID | 10096 | 10693 | 11270 | | | 11865 | 12587 | | 14054 | | | |
| | IDENTITY | 31% | 49% | 35% | | | 30% | 100% | | 31% | | | |
| | COVERAGE | 98% | 103% | 98% | | | 101% | 100% | | 98% | | | |
| SAU101574 | SeqID | | | | | | | 12588 | | | | | |
| | IDENTITY | | | | | | | 100% | | | | | |
| | COVERAGE | | | | | | | 101% | | | | | |
| SAU101575 | SeqID | | 10869 | | | | | 12589 | 13638 | | | | |
| | IDENTITY | | 31% | | | | | 100% | 27% | | | | |
| | COVERAGE | | 98% | | | | | 100% | 96% | | | | |
| SAU101576 | SeqID | | 10762 | | | | 12049 | 12554 | 13460 | | | | |
| | IDENTITY | | 32% | | | | 29% | 100% | 39% | | | | |
| | COVERAGE | | 93% | | | | 98% | 102% | 98% | | | | |
| SAU101586 | SeqID | | | | | | | 12598 | 13487 | | | | |
| | IDENTITY | | | | | | | 100% | 34% | | | | |
| | COVERAGE | | | | | | | 101% | 78% | | | | |
| SAU101592 | SeqID | 10249 | 10605 | 10987 | 11555 | 11741 | 11952 | 12406 | 13283 | 13950 | | | |
| | IDENTITY | 51% | 74% | 53% | 53% | 51% | 52% | 100% | 70% | 51% | | | |
| | COVERAGE | 101% | 100% | 100% | 100% | 101% | 101% | 100% | 100% | 101% | | | |
| SAU101599 | SeqID | | | | | | | 12478 | | | | | |
| | IDENTITY | | | | | | | 100% | | | | | |
| | COVERAGE | | | | | | | 100% | | | | | |
| SAU101610 | SeqID | 10449 | | | 11390 | | 12048 | 12629 | | 13816 | | | |
| | IDENTITY | 38% | | | 38% | | 40% | 100% | | 38% | | | |
| | COVERAGE | 105% | | | 101% | | 99% | 100% | | 105% | | | |
| SAU101612 | SeqID | | | | | | | 12637 | | | | | |
| | IDENTITY | | | | | | | 100% | | | | | |
| | COVERAGE | | | | | | | 100% | | | | | |
| SAU101614 | SeqID | 10167 | 10678 | 11262 | 11534 | | 11978 | 12649 | 13462 | 13851 | | | |
| | IDENTITY | 49% | 55% | 29% | 29% | | 39% | 100% | 53% | 48% | | | |
| | COVERAGE | 100% | 98% | 93% | 94% | | 95% | 100% | 99% | 100% | | | |
| SAU101616 | SeqID | 10186 | 10667 | | 11407 | 11695 | 11872 | 12432 | | 13903 | | | |
| | IDENTITY | 33% | 28% | | 32% | 29% | 34% | 100% | | 33% | | | |
| | COVERAGE | 102% | 99% | | 88% | 104% | 96% | | 100% | 100% | | | |
| SAU101622 | SeqID | 10162 | | | 11619 | 11710 | 12104 | 12430 | | 13832 | | | |
| | IDENTITY | 69% | | | 29% | 67% | 43% | 100% | | 70% | | | |
| | COVERAGE | 100% | | | 104% | 78% | 101% | 100% | | 100% | | | |
| SAU101624 | SeqID | 10193 | | 11255 | 11316 | | | 12429 | 13430 | 13752 | | | |
| | IDENTITY | 26% | | 27% | 38% | | | 100% | 26% | 26% | | | |
| | COVERAGE | 101% | | 106% | 97% | | | 100% | 103% | 107% | | | |
| SAU101630 | SeqID | | | | | | | 12410 | | | | | |
| | IDENTITY | | | | | | | 100% | | | | | |
| | COVERAGE | | | | | | | 100% | | | | | |
| SAU101632 | SeqID | | | | | | | 12407 | | | | | |
| | IDENTITY | | | | | | | 100% | | | | | |
| | COVERAGE | | | | | | | 100% | | | | | |
| SAU101637 | SeqID | | 10886 | | | | | 12201 | 13384 | | | | |
| | IDENTITY | | 44% | | | | | 100% | 38% | | | | |
| | COVERAGE | | 99% | | | | | 101% | 98% | | | | |
| SAU101641 | SeqID | 10223 | | | | | 11918 | 12193 | | | | | |
| | IDENTITY | 51% | | | | | 53% | 100% | | | | | |
| | COVERAGE | 92% | | | | | 95% | 100% | | | | | |
| SAU101651 | SeqID | | 10790 | | 11552 | | 12021 | 12491 | 13369 | | | | |
| | IDENTITY | | 38% | | 28% | | 34% | 100% | 42% | | | | |
| | COVERAGE | | 97% | | 89% | | 90% | 101% | 100% | | | | |
| SAU101652 | SeqID | | 10791 | | 11369 | | 12022 | 12492 | 13368 | | | | |
| | IDENTITY | | 62% | | 49% | | 50% | 100% | 56% | | | | |
| | COVERAGE | | 97% | | 91% | | 95% | 100% | 98% | | | | |
| SAU101653 | SeqID | | 10792 | | 11520 | | 12023 | 12493 | 13367 | | | | |
| | IDENTITY | | 73% | | 46% | | 49% | 100% | 63% | | | | |
| | COVERAGE | | 100% | | 100% | | 100% | 100% | 100% | | | | |
| SAU101655 | SeqID | 10205 | 10793 | | | | 11896 | 12494 | 13334 | | | | |
| | IDENTITY | 31% | 50% | | | | 30% | 100% | 33% | | | | |
| | COVERAGE | 84% | 97% | | | | 83% | 100% | 93% | | | | |
| SAU101663 | SeqID | | | | | | | 12261 | | | | | |
| | IDENTITY | | | | | | | 100% | | | | | |
| | COVERAGE | | | | | | | 100% | | | | | |
| SAU101664 | SeqID | 10202 | 10512 | 11138 | | | 11863 | 12262 | 13685 | 13823 | | | |
| | IDENTITY | 37% | 41% | 36% | | | 38% | 100% | 38% | 36% | | | |
| | COVERAGE | 98% | 97% | 108% | | | 106% | 101% | 105% | 98% | | | |
| SAU101674 | SeqID | 10067 | | | | | 11846 | 12594 | | 14082 | | | |
| | IDENTITY | 27% | | | | | 27% | 100% | | 27% | | | |
| | COVERAGE | 103% | | | | | 101% | 100% | | 103% | | | |
| SAU101679 | SeqID | 10190 | 10644 | 11055 | 11398 | | 12105 | 12593 | 13264 | 13756 | | | |
| | IDENTITY | 41% | 53% | 42% | 36% | | 45% | 100% | 45% | 40% | | | |
| | COVERAGE | 90% | 100% | 99% | 86% | | 90% | 100% | 98% | 90% | | | |
| SAU101681 | SeqID | 10464 | 10746 | | | | 11861 | 12592 | 13419 | 13987 | | | |
| | IDENTITY | 39% | 46% | | | | 31% | 100% | 44% | 40% | | | |
| | COVERAGE | 100% | 102% | | | | 95% | 100% | 102% | 97% | | | |
| SAU101682 | SeqID | 10156 | 10670 | 11265 | | | | 12591 | 13488 | 13884 | | | |
| | IDENTITY | 28% | 30% | 28% | | | | 100% | 34% | 26% | | | |
| | COVERAGE | 94% | 96% | 102% | | | | 100% | 80% | 94% | | | |
| SAU101685 | SeqID | | 10590 | | | | 11920 | 12152 | 13396 | | | | |
| | IDENTITY | | 26% | | | | 37% | 100% | 56% | | | | |
| | COVERAGE | | 88% | | | | 97% | 100% | 100% | | | | |
| SAU101717 | SeqID | 10129 | 10586 | 11027 | 11610 | | 11890 | 12131 | 13352 | 14070 | | | |
| | IDENTITY | 33% | 51% | 35% | 31% | | 38% | 100% | 49% | 34% | | | |
| | COVERAGE | 101% | 100% | 93% | 70% | | 99% | 100% | 93% | 101% | | | |
| SAU101724 | SeqID | 10309 | 10588 | 11268 | 11337 | | 12015 | 12136 | 13678 | 13772 | | | |
| | IDENTITY | 44% | 44% | 41% | 36% | | 43% | 100% | 45% | 43% | | | |
| | COVERAGE | 97% | 99% | 97% | 87% | | 80% | 100% | 98% | 97% | | | |
| SAU101726 | SeqID | 10130 | 10664 | 11026 | 11461 | | 11889 | 12134 | 13550 | 14071 | | | |
| | IDENTITY | 37% | 50% | 42% | 36% | | 40% | 100% | 48% | 41% | | | |
| | COVERAGE | 101% | 100% | 101% | 101% | | 100% | 100% | 100% | 77% | | | |
| SAU101727 | SeqID | | 10665 | | | | | 12133 | 13551 | | | | |
| | IDENTITY | | 50% | | | | | 100% | 49% | | | | |
| | COVERAGE | | 101% | | | | | 101% | 101% | | | | |
| SAU101728 | SeqID | 10019 | 10666 | 11053 | | 11734 | 11800 | 12132 | 13182 | 14015 | | | |
| | IDENTITY | 34% | 54% | 35% | | 35% | 34% | 100% | 53% | 34% | | | |
| | COVERAGE | 86% | 95% | 88% | | 85% | 90% | 100% | 94% | 86% | | | |
| SAU101736 | SeqID | 10225 | | | | | 11817 | 12519 | | 13958 | | | |
| | IDENTITY | 28% | | | | | 38% | 100% | | 29% | | | |
| | COVERAGE | 72% | | | | | 99% | | | | | 100% | 72% |
| SAU101737 | SeqID | | | | 11405 | | 11817 | 12518 | | | | | |
| | IDENTITY | | | | 32% | | 30% | 100% | | | | | |
| | COVERAGE | | | | 78% | | 96% | 101% | | | | | |
| SAU101744 | SeqID | | 10562 | | | | | 12367 | | | | | |
| | IDENTITY | | 44% | | | | | 100% | | | | | |
| | COVERAGE | | 101% | | | | | 100% | | | | | |
| SAU101751 | SeqID | 10474 | 10606 | | | 11671 | | 12448 | 13165 | 13706 | | | |
| | IDENTITY | 30% | 46% | | | 30% | | 100% | 45% | 31% | | | |
| | COVERAGE | 85% | 100% | | | 82% | | 100% | 99% | 79% | | | |
| SAU101752 | SeqID | 10438 | 10626 | 11037 | 11410 | | 11997 | 12447 | 13187 | 14043 | | | |
| | IDENTITY | 46% | 75% | 47% | 40% | | 45% | 100% | 69% | 46% | | | |
| | COVERAGE | 115% | 99% | 114% | 120% | | 116% | 100% | 99% | 115% | | | |
| SAU101754 | SeqID | 10439 | 10627 | 11036 | 11571 | | 5179 | 12446 | 13646 | 14042 | | | |
| | IDENTITY | 46% | 72% | 46% | 53% | | 46% | 100% | 68% | 46% | | | |
| | COVERAGE | 116% | 100% | 117% | 80% | | 118% | 100% | 101% | 116% | | | |
| SAU101756 | SeqID | 10365 | 10479 | 11062 | 11409 | | 5178 | 12445 | 13231 | 13967 | | | |
| | IDENTITY | 65% | 83% | 66% | 65% | | 68% | 100% | 82% | 65% | | | |
| | COVERAGE | 91% | 93% | 91% | 91% | | 91% | 101% | 93% | 93% | | | |
| SAU101771 | SeqID | 10220 | 10784 | 11276 | | 11765 | 11950 | 12350 | 13280 | 13934 | | | |
| | IDENTITY | 43% | 65% | 37% | | 35% | 36% | 100% | 67% | 41% | | | |
| | COVERAGE | 91% | 101% | 77% | | 82% | 80% | 101% | 98% | 91% | | | |
| SAU101772 | SeqID | 10240 | 10785 | 11275 | 11294 | | 11925 | 12351 | 13281 | 13863 | | | |
| | IDENTITY | 50% | 63% | 51% | 27% | | 38% | 100% | 61% | 48% | | | |
| | COVERAGE | 100% | 101% | 100% | 77% | | 100% | 100% | 101% | 84% | | | |
| SAU101777 | SeqID | | 10673 | | 11448 | | | 12352 | 13176 | | | | |
| | IDENTITY | | 64% | | 43% | | | 100% | 62% | | | | |
| | COVERAGE | | 97% | | 88% | | | 100% | 98% | | | | |
| SAU101781 | SeqID | | 10495 | | | | 11917 | 12353 | 13308 | | | | |
| | IDENTITY | | 67% | | | | 38% | 100% | 28% | | | | |
| | COVERAGE | | | 99% | | | | 93% | 100% | 85% | | | |
| SAU101782 | SeqID | | 10496 | | | 11689 | 11916 | 12354 | 13309 | | | | |
| | IDENTITY | | 75% | | | 44% | 41% | 100% | 40% | | | | |
| | COVERAGE | | 100% | | | 89% | 99% | 100% | 96% | | | | |
| SAU101784 | SeqID | 10037 | 10498 | 11208 | | 11700 | 11866 | 12355 | 13563 | 13900 | | | |
| | IDENTITY | 44% | 65% | 45% | | 35% | 42% | 100% | 37% | 44% | | | |
| | COVERAGE | 97% | 100% | 97% | | 92% | 99% | 100% | 99% | 97% | | | |
| SAU101790 | SeqID | 10350 | 10524 | 11106 | 11437 | | 5170 | 12215 | 13207 | | | | |
| | IDENTITY | 51% | 81% | 55% | 48% | | 55% | 100% | 79% | | | | |
| | COVERAGE | 86% | 99% | 86% | 86% | | 90% | 101% | 99% | | | | |
| SAU101791 | SeqID | 10349 | 10525 | 11107 | 11436 | | 5169 | 12216 | 13208 | 14108 | | | |
| | IDENTITY | 67% | 90% | 69% | 62% | | 66% | 100% | 89% | 67% | | | |
| | COVERAGE | 101% | 101% | 101% | 100% | | 100% | 101% | 101% | 102% | | | |
| SAU101792 | SeqID | 10348 | 10526 | 11108 | | | 5168 | 12217 | 13209 | 14107 | | | |
| | IDENTITY | 53% | 66% | 52% | | | 49% | 100% | 68% | 50% | | | |
| | COVERAGE | 96% | 94% | 95% | | | 97% | 101% | 94% | 96% | | | |
| SAU101793 | SeqID | 10347 | 10527 | 11109 | 11589 | 11654 | 5167 | 12218 | 13210 | 14106 | | | |
| | IDENTITY | 64% | 85% | 65% | 51% | 64% | 63% | 100% | 79% | 64% | | | |
| | COVERAGE | 100% | 101% | 99% | 99% | 101% | 99% | 101% | 100% | 101% | | | |
| SAU101795 | SeqID | 10345 | 10528 | 11111 | 11435 | | 5165 | 12219 | 13212 | 14104 | | | |
| | IDENTITY | 51% | 79% | 47% | 44% | | 44% | 100% | 76% | 51% | | | |
| | COVERAGE | 99% | 101% | 99% | 98% | | 100% | 101% | 101% | 101% | | | |
| SAU101797 | SeqID | 10343 | 10530 | 11113 | 11433 | | 5163 | 12221 | 13214 | 14102 | | | |
| | IDENTITY | 45% | 68% | 41% | 41% | | 48% | 100% | 66% | 46% | | | |
| | COVERAGE | 100% | 101% | 99% | 93% | | 96% | 101% | 101% | 101% | | | |
| SAU101798 | SeqID | 10342 | 10531 | 11114 | 11432 | | 5162 | 12222 | 13215 | 14101 | | | |
| | IDENTITY | 55% | 72% | 55% | 62% | | 52% | 100% | 66% | 55% | | | |
| | COVERAGE | 99% | 95% | 99% | 87% | | 99% | 101% | 96% | 99% | | | |
| SAU101799 | SeqID | 10341 | 10532 | 11115 | | | 5161 | 12223 | 13216 | | | | |
| | IDENTITY | 51% | 62% | 42% | | | 42% | 100% | 69% | | | | |
| | COVERAGE | 100% | 102% | 100% | | | 97% | 102% | 98% | | | | |
| SAU101800 | SeqID | 10340 | 10534 | 11116 | 11431 | | 5160 | 12225 | 13217 | 14099 | | | |
| | IDENTITY | 47% | 79% | 46% | 40% | | 42% | 100% | 84% | 47% | | | |
| | COVERAGE | 99% | 101% | 99% | 90% | | 99% | 101% | 101% | 99% | | | |
| SAU101802 | SeqID | 10075 | 10536 | 11008 | 11348 | 11633 | 11942 | 12227 | 13219 | 13717 | | | |
| | IDENTITY | 48% | 64% | 52% | 31% | 47% | 53% | 100% | 56% | 47% | | | |
| | COVERAGE | 97% | 97% | 97% | 93% | 97% | 84% | 100% | 96% | 97% | | | |
| SAU101803 | SeqID | 10111 | 10537 | 11052 | 11429 | 11651 | 11876 | 12228 | 13220 | 14010 | | | |
| | IDENTITY | 71% | 84% | 71% | 60% | 70% | 71% | 100% | 82% | 70% | | | |
| | COVERAGE | 97% | 101% | 97% | 100% | 101% | 97% | 101% | 101% | 101% | | | |
| SAU101805 | SeqID | 10337 | 10539 | 11119 | 11427 | | 11990 | 12229 | 13221 | 14097 | | | |
| | IDENTITY | 53% | 75% | 52% | 58% | | 60% | 100% | 74% | 52% | | | |
| | COVERAGE | 96% | 101% | 99% | 99% | | 96% | 101% | 101% | 96% | | | |
| SAU101806 | SeqID | 10336 | 10540 | 11120 | 11426 | | 11989 | 12230 | 13222 | 14096 | | | |
| | IDENTITY | 62% | 85% | 64% | 60% | | 61% | 100% | 85% | 63% | | | |
| | COVERAGE | 100% | 101% | 100% | 102% | | 100% | 101% | 92% | 101% | | | |
| SAU101807 | SeqID | 10334 | 10541 | 11122 | 11583 | | 11987 | 12231 | 13223 | 14094 | | | |
| | IDENTITY | 42% | 71% | 42% | 37% | | 42% | 100% | 58% | 42% | | | |
| | COVERAGE | 99% | 100% | 99% | 94% | | 99% | 100% | 99% | 99% | | | |
| SAU101808 | SeqID | 10333 | 10542 | 11123 | 11582 | 11627 | 5158 | 12232 | 13224 | 14093 | | | |
| | IDENTITY | 48% | 65% | 49% | 46% | 48% | 45% | 100% | 67% | 48% | | | |
| | COVERAGE | 98% | 103% | 98% | 99% | 78% | 98% | 101% | 106% | 98% | | | |
| SAU101810 | SeqID | 10053 | 10544 | 11229 | 11625 | 11666 | 11909 | 12233 | 13441 | 14110 | | | |
| | IDENTITY | 35% | 52% | 34% | 32% | 36% | 33% | 100% | 47% | 36% | | | |
| | COVERAGE | 76% | 88% | 78% | 77% | 73% | 72% | 100% | 88% | 73% | | | |
| SAU101811 | SeqID | 10196 | 10545 | 11068 | 11463 | 11666 | 11888 | 12234 | 13440 | 13721 | | | |
| | IDENTITY | 38% | 49% | 33% | 32% | 33% | 32% | 100% | 45% | 34% | | | |
| | COVERAGE | 78% | 87% | 82% | 82% | 83% | 82% | 100% | 87% | 76% | | | |
| SAU101814 | SeqID | 10327 | 10602 | 11241 | 11471 | 11655 | 5188 | 12237 | 13356 | 13729 | | | |
| | IDENTITY | 58% | 69% | 57% | 47% | 56% | 55% | 100% | 65% | 56% | | | |
| | COVERAGE | 94% | 96% | 94% | 92% | 71% | 97% | 101% | 99% | 94% | | | |
| SAU101815 | SeqID | 10326 | | 11240 | 11288 | | 12016 | 12238 | 13361 | 13732 | | | |
| | IDENTITY | 49% | | 48% | 46% | | 53% | 100% | 69% | 51% | | | |
| | COVERAGE | 98% | | 98% | 93% | | 93% | 101% | 99% | 99% | | | |
| SAU101818 | SeqID | | | 11231 | 11307 | | 11814 | 12369 | 13494 | | | | |
| | IDENTITY | | | 32% | 33% | | 31% | 100% | 35% | | | | |
| | COVERAGE | | | 95% | 90% | | 96% | 101% | 93% | | | | |
| SAU101824 | SeqID | 10158 | | | | | 12004 | 12371 | | | | | |
| | IDENTITY | 33% | | | | | 28% | 100% | | | | | |
| | COVERAGE | 71% | | | | | 75% | 100% | | | | | |
| SAU101833 | SeqID | 10207 | 10747 | 11040 | 11481 | | 11794 | 12373 | 13388 | 13775 | | | |
| | IDENTITY | 42% | 49% | 28% | 44% | | 35% | 100% | 46% | 44% | | | |
| | COVERAGE | 100% | 102% | 95% | 107% | | 117% | 100% | 103% | 89% | | | |
| SAU101839 | SeqID | 10398 | 10849 | 11236 | | | 12100 | 12495 | 13291 | 13924 | | | |
| | IDENTITY | 30% | 33% | 32% | | | 25% | 100% | 32% | 28% | | | |
| | COVERAGE | 94% | 78% | 90% | | | 98% | 100% | 83% | 94% | | | |
| SAU101842 | SeqID | 10105 | 10942 | 11075 | 11376 | 11723 | 11855 | 12510 | 13445 | 13999 | | | |
| | IDENTITY | 45% | 70% | 33% | 48% | 33% | 47% | 100% | 65% | 45% | | | |
| | COVERAGE | 98% | 95% | 95% | 99% | 94% | 97% | 100% | 82% | 99% | | | |
| SAU101845 | SeqID | 10231 | 10739 | | 11567 | | 11899 | 12506 | 13544 | 13953 | | | |
| | IDENTITY | 30% | 47% | | 40% | | 26% | 100% | 43% | 28% | | | |
| | COVERAGE | 101% | 102% | | 102% | | 101% | 100% | 102% | 101% | | | |
| SAU101849 | SeqID | 10015 | 10740 | 11209 | 11472 | | 12058 | 12567 | 13379 | 13713 | | | |
| | IDENTITY | 56% | 77% | 54% | 56% | | 56% | 100% | 75% | 56% | | | |
| | COVERAGE | 103% | 99% | 103% | 101% | | 103% | 100% | 98% | 104% | | | |
| SAU101857 | SeqID | | | | | | | 12569 | | | | | |
| | IDENTITY | | | | | | | 100% | | | | | |
| | COVERAGE | | | | | | | 100% | | | | | |
| SAU101862 | SeqID | 10257 | 10817 | 10955 | 11334 | | 11802 | 12571 | 13305 | 13797 | | | |
| | IDENTITY | 40% | 63% | 40% | 33% | | 39% | 100% | 62% | 39% | | | |
| | COVERAGE | 98% | 100% | 98% | 101% | | 98% | 100% | 99% | 98% | | | |
| SAU101864 | SeqID | | | | | | | 12572 | | | | | |
| | IDENTITY | | | | | | | 100% | | | | | |
| | COVERAGE | | | | | | | 100% | | | | | |
| SAU101865 | SeqID | 10044 | 10834 | 11151 | 11417 | | 11938 | 12318 | 13227 | 13910 | | | |
| | IDENTITY | 43% | 58% | 45% | 40% | | 40% | 100% | 54% | 41% | | | |
| | COVERAGE | 85% | 88% | 88% | 87% | | 87% | 100% | 88% | 88% | | | |
| SAU101866 | SeqID | | 10835 | | | | 11873 | 12319 | 13586 | | | | |
| | IDENTITY | | 42% | | | | 29% | 100% | 40% | | | | |
| | COVERAGE | | 102% | | | | 99% | 100% | 100% | | | | |
| SAU101868 | SeqID | 10049 | 10733 | 11086 | 11305 | | 11813 | 12320 | 13228 | 13898 | | | |
| | IDENTITY | 45% | 56% | 45% | 42% | | 48% | 100% | 49% | 45% | | | |
| | COVERAGE | 101% | 99% | 101% | 96% | | 100% | 100% | 108% | 99% | | | |
| SAU101869 | SeqID | | 10734 | | | | | 12321 | 13668 | | | | |
| | IDENTITY | | 55% | | | | | 100% | 49% | | | | |
| | COVERAGE | | 100% | | | | | 100% | 101% | | | | |
| SAU101876 | SeqID | | | | | | | 12169 | | | | | |
| | IDENTITY | | | | | | | 100% | | | | | |
| | COVERAGE | | | | | | | 101% | | | | | |
| SAU101881 | SeqID | 10325 | | | | | 12081 | 12162 | | 13728 | | | |
| | IDENTITY | 42% | | | | | 41% | 100% | | 42% | | | |
| | COVERAGE | 98% | | | | | 97% | 100% | | 98% | | | |
| SAU101882 | SeqID | 10246 | 10824 | | | 11743 | 12080 | 12163 | | 13727 | | | |
| | IDENTITY | 33% | 30% | | | 31% | 31% | 100% | | 33% | | | |
| | COVERAGE | 96% | 89% | | | 73% | 94% | 100% | | 95% | | | |
| SAU101890 | SeqID | 10374 | | 11125 | | | 12091 | 12280 | | 13809 | | | |
| | IDENTITY | 53% | | 49% | | | 47% | 100% | | 53% | | | |
| | COVERAGE | 91% | | 92% | | | 93% | 100% | | 91% | | | |
| SAU101891 | SeqID | 10295 | 10766 | 11196 | 11483 | | 11791 | 12281 | 13413 | 13739 | | | |
| | IDENTITY | 63% | 72% | 62% | 60% | | 58% | 100% | 67% | 64% | | | |
| | COVERAGE | 91% | 91% | 90% | 90% | | 93% | 100% | 92% | 91% | | | |
| SAU101893 | SeqID | 10300 | 10724 | | | 11748 | 11981 | 12282 | 13290 | 13825 | | | |
| | IDENTITY | 46% | 47% | | | 41% | 35% | 100% | 40% | 43% | | | |
| | COVERAGE | 87% | 100% | | | 78% | 93% | 100% | 95% | 96% | | | |
| SAU101904 | SeqID | 10047 | 10648 | 11089 | 11451 | | 11935 | 12617 | 13345 | 13913 | | | |
| | IDENTITY | 34% | 38% | 33% | 31% | | 31% | 100% | 34% | 33% | | | |
| | COVERAGE | 98% | 101% | 102% | 105% | | 104% | 100% | 93% | 98% | | | |
| SAU101907 | SeqID | 10362 | 10482 | 11059 | 11415 | | 11995 | 12442 | 13171 | 13964 | | | |
| | IDENTITY | 75% | 90% | 76% | 74% | | 73% | 100% | 75% | 74% | | | |
| | COVERAGE | 100% | 101% | 100% | 101% | | 101% | 100% | 101% | 100% | | | |
| SAU101909 | SeqID | 10390 | | 11249 | 11346 | | 11789 | 12441 | | 14063 | | | |
| | IDENTITY | 41% | | 32% | 29% | | 36% | 100% | | 32% | | | |
| | COVERAGE | 99% | | 88% | 90% | | 93% | 100% | | 73% | | | |
| SAU101910 | SeqID | 10199 | | | | | 11818 | 12440 | | | | | |
| | IDENTITY | 56% | | | | | 60% | 100% | | | | | |
| | COVERAGE | 97% | | | | | 97% | 100% | | | | | |
| SAU101915 | SeqID | | 10838 | | | | | 12439 | | | | | |
| | IDENTITY | | 26% | | | | | 100% | | | | | |
| | COVERAGE | | 90% | | | | | 100% | | | | | |
| SAU101922 | SeqID | | | | | | | 12438 | | | | | |
| | IDENTITY | | | | | | | 100% | | | | | |
| | COVERAGE | | | | | | | 100% | | | | | |
| SAU101948 | SeqID | | | | | | | 12709 | | | | | |
| | IDENTITY | | | | | | | 100% | | | | | |
| | COVERAGE | | | | | | | 100% | | | | | |
| SAU101966 | SeqID | 10101 | 10561 | 11007 | 11538 | 11705 | 11897 | 12186 | | 14003 | | | |
| | IDENTITY | 45% | 31% | 32% | 37% | 43% | 45% | 100% | | 45% | | | |
| | COVERAGE | 88% | 91% | 92% | 86% | 88% | 88% | 101% | | 88% | | | |
| SAU101968 | SeqID | 10106 | 10568 | 11242 | 11480 | | 11965 | 12187 | | 13998 | | | |
| | IDENTITY | 30% | 31% | 33% | 27% | | 30% | 100% | | 31% | | | |
| | COVERAGE | 90% | 92% | 90% | 88% | | 83% | 100% | | 76% | | | |
| SAU101991 | SeqID | | 10938 | | | | | 12454 | 13500 | | | | |
| | IDENTITY | | 40% | | | | | 100% | 25% | | | | |
| | COVERAGE | | 101% | | | | | 101% | 80% | | | | |
| SAU101995 | SeqID | 10388 | 10939 | 11066 | 11575 | 11646 | 11957 | 12455 | 13386 | | | | |
| | IDENTITY | 46% | 47% | 49% | 58% | 46% | 57% | 100% | 51% | | | | |
| | COVERAGE | 72% | 78% | 73% | 72% | 72% | 76% | 100% | 74% | | | | |
| SAU101996 | SeqID | 10237 | 10940 | 10999 | 11325 | | 11901 | 12456 | 13455 | 13956 | | | |
| | IDENTITY | 38% | 64% | 36% | 38% | | 35% | 100% | 58% | 37% | | | |
| | COVERAGE | 98% | 99% | 98% | 98% | | 99% | 100% | 100% | 98% | | | |
| SAU101999 | SeqID | 10476 | 10941 | 11259 | 11304 | | 12035 | 12423 | 13241 | 13708 | | | |
| | IDENTITY | 48% | 61% | 46% | 49% | | 51% | 100% | 64% | 48% | | | |
| | COVERAGE | 97% | 98% | 98% | 91% | | 96% | 100% | 97% | 97% | | | |
| SAU102001 | SeqID | 10258 | 10628 | 11134 | 11489 | | 11787 | 12424 | 13636 | 14088 | | | |
| | IDENTITY | 47% | 58% | 47% | 43% | | 49% | 100% | 46% | 47% | | | |
| | COVERAGE | 105% | 98% | 106% | 105% | | 98% | 100% | 98% | 105% | | | |
| SAU102002 | SeqID | | | | | | | 12425 | | | | | |
| | IDENTITY | | | | | | | 100% | | | | | |
| | COVERAGE | | | | | | | 100% | | | | | |
| SAU102003 | SeqID | | | | | | | 12426 | | | | | |
| | IDENTITY | | | | | | | 100% | | | | | |
| | COVERAGE | | | | | | | 101% | | | | | |
| SAU102006 | SeqID | | | 11267 | 11555 | | | 12427 | 13260 | | | | |
| | IDENTITY | | | 44% | 28% | | | 100% | 47% | | | | |
| | COVERAGE | | | 92% | 74% | | | 101% | 105% | | | | |
| SAU102007 | SeqID | | | 11266 | | | | 12428 | 13258 | | | | |
| | IDENTITY | | | 60% | | | | 100% | 61% | | | | |
| | COVERAGE | | | 97% | | | | 100% | 97% | | | | |
| SAU102032 | SeqID | | | | | | 12086 | 12198 | | 13989 | | | |
| | IDENTITY | | | | | | 62% | 100% | | 58% | | | |
| | COVERAGE | | | | | | 99% | 100% | | 75% | | | |
| SAU102035 | SeqID | 10299 | 10933 | 10974 | 11514 | | 11860 | 12199 | 13360 | 13763 | | | |
| | IDENTITY | 60% | 50% | 26% | 29% | | 41% | 100% | 31% | 56% | | | |
| | COVERAGE | 98% | 99% | 85% | 84% | | 97% | 100% | 86% | 99% | | | |
| SAU102044 | SeqID | 10141 | 10916 | 11011 | 11344 | | 12041 | 12414 | 13447 | 13977 | | | |
| | IDENTITY | 56% | 67% | 59% | 50% | | 58% | 100% | 69% | 56% | | | |
| | COVERAGE | 100% | 102% | 100% | 101% | | 101% | 100% | 102% | 100% | | | |
| SAU102046 | SeqID | 10103 | 10723 | | | | 12089 | 12415 | | 14001 | | | |
| | IDENTITY | 32% | 28% | | | | 29% | 100% | | 29% | | | |
| | COVERAGE | 74% | 86% | | | | 90% | 100% | | 89% | | | |
| SAU102049 | SeqID | 10427 | 10518 | 10962 | 11291 | | 11784 | 12416 | 13652 | 13781 | | | |
| | IDENTITY | 36% | 39% | 49% | 40% | | 41% | 100% | 46% | 36% | | | |
| | COVERAGE | 101% | 99% | 97% | 99% | | 100% | 100% | 98% | 101% | | | |
| SAU102054 | SeqID | 10280 | 10494 | 11095 | 11356 | 11676 | 11856 | 12417 | | 13877 | | | |
| | IDENTITY | 53% | 50% | 55% | 51% | 53% | 55% | 100% | | 53% | | | |
| | COVERAGE | 100% | 79% | 100% | 100% | 70% | 100% | 100% | | 100% | | | |
| SAU102059 | SeqID | 10085 | 10771 | 11152 | 11622 | | 11969 | 12286 | 13226 | 14059 | | | |
| | IDENTITY | 43% | 72% | 43% | 40% | | 41% | 100% | 72% | 40% | | | |
| | COVERAGE | 107% | 100% | 107% | 102% | | 109% | 100% | 71% | 89% | | | |
| SAU102067 | SeqID | 10380 | 10564 | 11155 | | | 11795 | 12287 | 13407 | 13798 | | | |
| | IDENTITY | 32% | 52% | 31% | | | 28% | 100% | 44% | 31% | | | |
| | COVERAGE | 95% | 98% | 98% | | | 97% | 100% | 98% | 94% | | | |
| SAU102068 | SeqID | | 10680 | | | | | 12288 | | | | | |
| | IDENTITY | | 29% | | | | | 100% | | | | | |
| | COVERAGE | | 101% | | | | | 100% | | | | | |
| SAU102102 | SeqID | | | | | | | 12696 | | | | | |
| | IDENTITY | | | | | | | 100% | | | | | |
| | COVERAGE | | | | | | | 100% | | | | | |
| SAU102113 | SeqID | | 10641 | | | | | 12178 | | | | | |
| | IDENTITY | | 34% | | | | | 100% | | | | | |
| | COVERAGE | | 110% | | | | | 101% | | | | | |
| SAU102116 | SeqID | | 10642 | | | | | 12180 | 13480 | | | | |
| | IDENTITY | | 29% | | | | | 100% | 31% | | | | |
| | COVERAGE | | 85% | | | | | 100% | 81% | | | | |
| SAU102117 | SeqID | 10016 | 10643 | | 11604 | | 12027 | 12181 | 13481 | 13947 | | | |
| | IDENTITY | 43% | 61% | | 38% | | 42% | 100% | 55% | 41% | | | |
| | COVERAGE | 101% | 100% | | 102% | | 103% | 100% | 100% | 85% | | | |
| SAU102129 | SeqID | | 10859 | | | | | 12176 | 13400 | | | | |
| | IDENTITY | | 60% | | | | | 100% | 56% | | | | |
| | COVERAGE | | 98% | | | | | 100% | 99% | | | | |
| SAU102132 | SeqID | | 10760 | | | | | 12177 | 13304 | | | | |
| | IDENTITY | | 39% | | | | | 100% | 41% | | | | |
| | COVERAGE | | 101% | | | | | 100% | 101% | | | | |
| SAU102142 | SeqID | 10154 | | | | | | 12457 | | | | | |
| | IDENTITY | 37% | | | | | | 100% | | | | | |
| | COVERAGE | 99% | | | | | | 100% | | | | | |
| SAU102143 | SeqID | 10154 | | | | | | 12458 | | | | | |
| | IDENTITY | 32% | | | | | | 100% | | | | | |
| | COVERAGE | 100% | | | | | | 100% | | | | | |
| SAU102144 | SeqID | | | | | | | 12459 | | | | | |
| | IDENTITY | | | | | | | 100% | | | | | |
| | COVERAGE | | | | | | | 100% | | | | | |
| SAU102162 | SeqID | | | | | | | 12462 | | | | | |
| | IDENTITY | | | | | | | 100% | | | | | |
| | COVERAGE | | | | | | | 100% | | | | | |
| SAU102165 | SeqID | | | | | | | 12460 | | | | | |
| | IDENTITY | | | | | | | 100% | | | | | |
| | COVERAGE | | | | | | | 100% | | | | | |
| SAU102200 | SeqID | | | | | | | 12665 | | | | | |
| | IDENTITY | | | | | | | 100% | | | | | |
| | COVERAGE | | | | | | | | 101% | | | | |
| SAU102201 | SeqID | | | | | | | 12666 | | | | | |
| | IDENTITY | | | | | | | 100% | | | | | |
| | COVERAGE | | | | | | | 101% | | | | | |
| SAU102222 | SeqID | 10447 | 10797 | 10994 | 11358 | | 11986 | 12511 | 13192 | 13818 | | | |
| | IDENTITY | 58% | 68% | 58% | 52% | | 59% | 100% | 67% | 58% | | | |
| | COVERAGE | 99% | 99% | 99% | 99% | | 99% | 100% | 99% | 99% | | | |
| SAU102231 | SeqID | 10323 | 10798 | 11193 | | | 12020 | 12527 | 13561 | 13731 | | | |
| | IDENTITY | 41% | 50% | 42% | | | 38% | 100% | 46% | 41% | | | |
| | COVERAGE | 94% | 93% | 89% | | | 94% | 100% | 99% | 94% | | | |
| SAU102232 | SeqID | 10100 | 10799 | | | 11687 | | 12530 | 13562 | 14004 | | | |
| | IDENTITY | 36% | 40% | | | 35% | | 100% | 42% | 34% | | | |
| | COVERAGE | 75% | 79% | | | 74% | | 100% | 79% | 75% | | | |
| SAU102233 | SeqID | | 10800 | | | | | 12531 | 13496 | | | | |
| | IDENTITY | | 61% | | | | | 100% | 45% | | | | |
| | COVERAGE | | 98% | | | | | 100% | 91% | | | | |
| SAU102241 | SeqID | 10163 | 10845 | | | | | 12539 | | | | | |
| | IDENTITY | 28% | 43% | | | | | 100% | | | | | |
| | COVERAGE | 74% | 99% | | | | | 100% | | | | | |
| SAU102242 | SeqID | 10188 | 10847 | 10953 | 11600 | 11634 | 11907 | 12540 | 13593 | 13981 | | | |
| | IDENTITY | 47% | 72% | 44% | 38% | 47% | 47% | 100% | 70% | 47% | | | |
| | COVERAGE | 100% | 99% | 101% | 100% | 98% | 100% | 100% | 100% | 100% | | | |
| SAU102246 | SeqID | 10274 | 10854 | 11154 | 11476 | | 11932 | 12542 | 13313 | 13866 | | | |
| | IDENTITY | 59% | 74% | 60% | 54% | | 62% | 100% | 81% | 58% | | | |
| | COVERAGE | 99% | 100% | 97% | 96% | | 100% | 100% | 101% | 99% | | | |
| SAU102247 | SeqID | | | | | | | 12543 | 13180 | | | | |
| | IDENTITY | | | | | | | 100% | 28% | | | | |
| | COVERAGE | | | | | | | 101% | 74% | | | | |
| SAU102252 | SeqID | 10300 | 10677 | | | 11748 | 11981 | 12241 | 13290 | 13825 | | | |
| | IDENTITY | 39% | 48% | | | 39% | 37% | 100% | 43% | 41% | | | |
| | COVERAGE | 79% | 93% | | | 73% | 91% | 100% | 95% | 98% | | | |
| SAU102256 | SeqID | 10451 | | | 11515 | | | 12243 | 13531 | | | | |
| | IDENTITY | 33% | | | 32% | | | 100% | 75% | | | | |
| | COVERAGE | 97% | | | 97% | | | 101% | 101% | | | | |
| SAU102257 | SeqID | 10451 | | | 11515 | | | 12244 | 13274 | | | | |
| | IDENTITY | 38% | | | 29% | | | 100% | 85% | | | | |
| | COVERAGE | 81% | | | 75% | | | 101% | 101% | | | | |
| SAU102259 | SeqID | | 10844 | | | | | 12245 | 13519 | 13782 | | | |
| | IDENTITY | | 65% | | | | | 100% | 72% | 25% | | | |
| | COVERAGE | | 97% | | | | | 100% | 97% | 87% | | | |
| SAU102260 | SeqID | 10182 | 10646 | | | 11682 | | 12246 | 13275 | 13984 | | | |
| | IDENTITY | 34% | 37% | | | 32% | | 100% | 83% | 32% | | | |
| | COVERAGE | 96% | 87% | | | 96% | | 101% | 100% | 87% | | | |
| SAU102261 | SeqID | 10183 | 10731 | | | | | 12247 | 13276 | 13983 | | | |
| | IDENTITY | 25% | 30% | | | | | 100% | 74% | 26% | | | |
| | COVERAGE | 79% | 80% | | | | | 100% | 99% | 79% | | | |
| SAU102262 | SeqID | 10270 | 10759 | | | 11724 | | 12248 | 13277 | 13881 | | | |
| | IDENTITY | 35% | 39% | | | 31% | | 100% | 82% | 34% | | | |
| | COVERAGE | 104% | 103% | | | | 84% | | 100% | 100% | 104% | | |
| SAU102264 | SeqID | 10160 | | | | | 5103 | 12250 | | 13830 | | | |
| | IDENTITY | 45% | | | | | 44% | 100% | | 43% | | | |
| | COVERAGE | 100% | | | | | 100% | 100% | | 101% | | | |
| SAU102265 | SeqID | | | | | | 11926 | 12251 | | | | | |
| | IDENTITY | | | | | | 37% | 100% | | | | | |
| | COVERAGE | | | | | | 100% | 100% | | | | | |
| SAU102268 | SeqID | | | | | | | 12252 | | | | | |
| | IDENTITY | | | | | | | 100% | | | | | |
| | COVERAGE | | | | | | | 101% | | | | | |
| SAU102270 | SeqID | | | | | | | 12253 | | | | | |
| | IDENTITY | | | | | | | 100% | | | | | |
| | COVERAGE | | | | | | | 100% | | | | | |
| SAU102280 | SeqID | | | | | | | 12378 | | | | | |
| | IDENTITY | | | | | | | 100% | | | | | |
| | COVERAGE | | | | | | | 100% | | | | | |
| SAU102281 | SeqID | 10316 | | 11227 | 11469 | | 12054 | 12384 | 13497 | 13762 | | | |
| | IDENTITY | 45% | | 48% | 39% | | 45% | 100% | 61% | 44% | | | |
| | COVERAGE | 99% | | 99% | 100% | | 99% | 100% | 100% | 99% | | | |
| SAU102283 | SeqID | 10260 | 10875 | 10982 | 11560 | | 11945 | 12119 | 13251 | 14086 | | | |
| | IDENTITY | 41% | 59% | 43% | 41% | | 41% | 100% | 54% | 41% | | | |
| | COVERAGE | 88% | 88% | 88% | 92% | | 95% | 102% | 88% | 88% | | | |
| SAU102284 | SeqID | | | | | | | 12389 | | | | | |
| | IDENTITY | | | | | | | 100% | | | | | |
| | COVERAGE | | | | | | | 100% | | | | | |
| SAU102286 | SeqID | 10385 | 10595 | | | | | 12393 | 13688 | | | | |
| | IDENTITY | 37% | 42% | | | | | 100% | 39% | | | | |
| | COVERAGE | 104% | 99% | | | | | 100% | 101% | | | | |
| SAU102287 | SeqID | 10220 | 10594 | 11025 | | 11663 | 11925 | 12398 | 13427 | 13934 | | | |
| | IDENTITY | 42% | 45% | 40% | | 39% | 41% | 100% | 41% | 39% | | | |
| | COVERAGE | 81% | 95% | 88% | | 89% | 84% | 101% | 94% | 83% | | | |
| SAU102292 | SeqID | 10399 | 10579 | 11018 | 11455 | 11758 | 12111 | 12368 | 13230 | 14065 | | | |
| | IDENTITY | 41% | 59% | 40% | 37% | 41% | 42% | 100% | 57% | 41% | | | |
| | COVERAGE | 101% | 100% | 101% | 100% | 101% | 101% | 100% | 94% | 101% | | | |
| SAU102294 | SeqID | | | | | | | 12610 | | | | | |
| | IDENTITY | | | | | | | 100% | | | | | |
| | COVERAGE | | | | | | | 100% | | | | | |
| SAU102297 | SeqID | 10405 | 10912 | 11063 | 11303 | | 12117 | 12704 | 13686 | 14066 | | | |
| | IDENTITY | 52% | 66% | 51% | 46% | | 50% | 100% | 64% | 48% | | | |
| | COVERAGE | 99% | 100% | 100% | 99% | | 98% | 100% | 100% | 77% | | | |
| SAU102298 | SeqID | 10404 | 10914 | 11031 | | 11686 | 12116 | 12705 | 13255 | | | | |
| | IDENTITY | 36% | 62% | 33% | | 35% | 28% | 100% | 54% | | | | |
| | COVERAGE | 72% | 99% | 87% | 89% | 87% | | 100% | 100% | | | | |
| SAU102308 | SeqID | 10077 | 10577 | 11248 | 11625 | 11732 | 12032 | 12706 | 13350 | 13995 | | | |
| | IDENTITY | 38% | 46% | 37% | 33% | 39% | 38% | 100% | 45% | 39% | | | |
| | COVERAGE | 88% | 100% | 86% | 87% | 88% | 90% | 100% | 100% | 95% | | | |
| SAU102318 | SeqID | 10122 | 10795 | | | | 11806 | 12707 | 13242 | 14039 | | | |
| | IDENTITY | 32% | 75% | | | | 37% | 100% | 63% | 31% | | | |
| | COVERAGE | 90% | 97% | | | | 72% | 100% | 97% | 89% | | | |
| SAU102333 | SeqID | 10057 | 10550 | | | | 12102 | 12657 | 13316 | 13829 | | | |
| | IDENTITY | 41% | 43% | | | | 40% | 100% | 31% | 38% | | | |
| | COVERAGE | 96% | 97% | | | | 96% | 100% | 90% | 95% | | | |
| SAU102334 | SeqID | 10056 | | | | | 12101 | 12658 | | | | | |
| | IDENTITY | 50% | | | | | 50% | 100% | | | | | |
| | COVERAGE | 91% | | | | | 92% | 100% | | | | | |
| SAU102336 | SeqID | | | | | | | 12659 | | | | | |
| | IDENTITY | | | | | | | 100% | | | | | |
| | COVERAGE | | | | | | | 101% | | | | | |
| SAU102340 | SeqID | | | | | | | 12660 | | | | | |
| | IDENTITY | | | | | | | 100% | | | | | |
| | COVERAGE | | | | | | | 100% | | | | | |
| SAU102345 | SeqID | | | | | | 11843 | 12655 | | | | | |
| | IDENTITY | | | | | | 37% | 100% | | | | | |
| | COVERAGE | 86% | | | | | | 101% | | | | | |
| SAU102350 | SeqID | | | | | | | 12433 | | | | | |
| | IDENTITY | | | | | | | 100% | | | | | |
| | COVERAGE | | | | | | | 101% | | | | | |
| SAU102352 | SeqID | | 10657 | | | | | 12434 | 13426 | | | | |
| | IDENTITY | | 55% | | | | | 100% | 39% | | | | |
| | COVERAGE | | 100% | | | | | 100% | 91% | | | | |
| SAU102355 | SeqID | | 10726 | | | | | 12435 | | | | | |
| | IDENTITY | | 39% | | | | | 100% | | | | | |
| | COVERAGE | | 87% | | | | | 100% | | | | | |
| SAU102356 | SeqID | 10227 | 10669 | 11203 | 11546 | | 11805 | 12436 | 13324 | 13960 | | | |
| | IDENTITY | 43% | 60% | 45% | 48% | | 43% | 100% | 56% | 43% | | | |
| | COVERAGE | 95% | 100% | 95% | 98% | | 95% | 100% | 99% | 95% | | | |
| SAU102378 | SeqID | | | | | | | 12437 | | | | | |
| | IDENTITY | | | | | | | 100% | | | | | |
| | COVERAGE | | | | | | | 100% | | | | | |
| SAU102380 | SeqID | | | | | | 11870 | 12265 | | | | | |
| | IDENTITY | | | | | | 32% | 100% | | | | | |
| | COVERAGE | | | | | | 71% | 100% | | | | | |
| SAU102388 | SeqID | 10367 | | 11157 | 11386 | | 11808 | 12267 | | 13802 | | | |
| | IDENTITY | 36% | | 33% | 27% | | 39% | 100% | | 36% | | | |
| | COVERAGE | 96% | | 90% | 101% | | 99% | 100% | | 96% | | | |
| SAU102389 | SeqID | 10063 | 10547 | 10988 | | | 11837 | 12268 | 13395 | 13917 | | | |
| | IDENTITY | 33% | 59% | 31% | | | 36% | 100% | 35% | 33% | | | |
| | COVERAGE | 99% | 97% | 97% | | | 95% | 100% | 98% | 99% | | | |
| SAU102390 | SeqID | 10192 | | | | 11678 | | 12269 | | 13753 | | | |
| | IDENTITY | 41% | | | | 26% | | 100% | | 42% | | | |
| | COVERAGE | 100% | | | | 97% | | 101% | | 100% | | | |
| SAU102392 | SeqID | 10131 | 10500 | | | 11673 | 11951 | 12270 | 13474 | | | | |
| | IDENTITY | 50% | 42% | | | 32% | 42% | 100% | 42% | | | | |
| | COVERAGE | 73% | 80% | | | 80% | 74% | 100% | 76% | | | | |
| SAU102394 | SeqID | | 10807 | | | | | 12271 | | | | | |
| | IDENTITY | | 32% | | | | | 100% | | | | | |
| | COVERAGE | | 102% | | | | | 100% | | | | | |
| SAU102396 | SeqID | 10243 | 10809 | | | | | 12272 | 13467 | 13794 | | | |
| | IDENTITY | 37% | 62% | | | | | 100% | 27% | 37% | | | |
| | COVERAGE | 101% | 99% | | | | | 100% | 98% | 98% | | | |
| SAU102401 | SeqID | | | | | | | 12209 | | | | | |
| | IDENTITY | | | | | | | 100% | | | | | |
| | COVERAGE | | | | | | | | 100% | | | | |
| SAU102417 | SeqID | | 10934 | | | | 12068 | 12204 | | | | | |
| | IDENTITY | | 31% | | | | 25% | 100% | | | | | |
| | COVERAGE | | 79% | | | | 72% | 100% | | | | | |
| SAU102418 | SeqID | | | | | 11760 | | 12205 | | | | | |
| | IDENTITY | | | | | 25% | | 100% | | | | | |
| | COVERAGE | | | | | 89% | | 100% | | | | | |
| SAU102420 | SeqID | | | | | | | 12206 | | | | | |
| | IDENTITY | | | | | | | 100% | | | | | |
| | COVERAGE | | | | | | | 100% | | | | | |
| SAU102422 | SeqID | 10308 | | | | 11665 | 11977 | 12207 | | 13776 | | | |
| | IDENTITY | 30% | | | | 30% | 27% | 100% | | 31% | | | |
| | COVERAGE | 92% | | | | 72% | 93% | 100% | | 92% | | | |
| SAU102423 | SeqID | | | 11084 | 11491 | | 12099 | 12208 | | | | | |
| | IDENTITY | | | 27% | 25% | | 27% | 100% | | | | | |
| | COVERAGE | | | 94% | 92% | | 93% | 100% | | | | | |
| SAU102433 | SeqID | 10395 | 10908 | 11167 | 11616 | | 11772 | 12701 | 13552 | | | | |
| | IDENTITY | 42% | 51% | 39% | 37% | | 52% | 100% | 44% | | | | |
| | COVERAGE | 101% | 100% | 100% | 73% | | 72% | 100% | 98% | | | | |
| SAU102434 | SeqID | 10394 | 10907 | 11166 | | | 11773 | 12700 | 13446 | 13921 | | | |
| | IDENTITY | 26% | 44% | 28% | | | 26% | 100% | 40% | 27% | | | |
| | COVERAGE | 99% | 100% | 99% | | | 100% | 100% | 101% | 99% | | | |
| SAU102437 | SeqID | 10393 | 10952 | 11057 | 11330 | | 11774 | 12695 | 13420 | 13920 | | | |
| | IDENTITY | 55% | 67% | 57% | 51% | | 55% | 100% | 64% | 56% | | | |
| | COVERAGE | 86% | 99% | 88% | 86% | | 87% | 100% | 99% | 86% | | | |
| SAU102440 | SeqID | | | | | 12085 | 12692 | | 13990 | | | | |
| | IDENTITY | | | | | 41% | 100% | | 39% | | | | |
| | COVERAGE | | | | | 98% | 100% | | 99% | | | | |
| SAU102447 | SeqID | | 10947 | | | | | 12685 | 13436 | | | | |
| | IDENTITY | | 38% | | | | | 100% | 32% | | | | |
| | COVERAGE | | 98% | | | | | 100% | 98% | | | | |
| SAU102448 | SeqID | 10460 | 10946 | 11049 | 11332 | | 12073 | 12681 | 13435 | 13860 | | | |
| | IDENTITY | 32% | 55% | 31% | 35% | | 34% | 100% | 46% | 32% | | | |
| | COVERAGE | 101% | 102% | 101% | 101% | | 101% | 101% | 102% | 102% | | | |
| SAU102449 | SeqID | 10445 | 10945 | 11253 | 11444 | 11731 | 12072 | 12677 | 13434 | 14028 | | | |
| | IDENTITY | 45% | 55% | 43% | 35% | 43% | 44% | 100% | 51% | 45% | | | |
| | COVERAGE | 97% | 98% | 98% | 99% | 76% | 97% | 100% | 100% | 97% | | | |
| SAU102450 | SeqID | 10456 | 10943 | 11264 | 11487 | | 12076 | 12675 | 13237 | 13857 | | | |
| | IDENTITY | 47% | 70% | 46% | 43% | | 47% | 100% | 68% | 47% | | | |
| | COVERAGE | 100% | 100% | 100% | | 99% | 99% | 100% | 100% | 100% | | | |
| SAU102452 | SeqID | 10420 | 10748 | 11143 | 11478 | 11629 | 11820 | 12674 | 13265 | 13783 | | | |
| | IDENTITY | 41% | 70% | 37% | 32% | 40% | 40% | 100% | 62% | 38% | | | |
| | COVERAGE | 97% | 98% | 97% | 97% | 94% | 97% | 100% | 100% | 99% | | | |
| SAU102453 | SeqID | | 10749 | | | | 12107 | 12669 | 13266 | | | | |
| | IDENTITY | | 43% | | | | 29% | 100% | 41% | | | | |
| | COVERAGE | | 101% | | | | 70% | 100% | 71% | | | | |
| SAU102460 | SeqID | 10063 | 10547 | 10988 | | | 11837 | 12171 | 13395 | 13917 | | | |
| | IDENTITY | 34% | 35% | 34% | | | 34% | 100% | 34% | 34% | | | |
| | COVERAGE | 98% | 100% | 100% | | | 100% | 100% | 101% | 98% | | | |
| SAU102469 | SeqID | 10217 | | | | | 12172 | | | | | | |
| | IDENTITY | 58% | | | | | 100% | | | | | | |
| | COVERAGE | 98% | | | | | 100% | | | | | | |
| SAU102473 | SeqID | | 10868 | | | | | 12173 | 13475 | | | | |
| | IDENTITY | | 28% | | | | | 100% | 35% | | | | |
| | COVERAGE | | 88% | | | | | 100% | 83% | | | | |
| SAU102474 | SeqID | | 10713 | 10971 | | | | 12174 | 13476 | 14025 | | | |
| | IDENTITY | | 26% | 26% | | | | 100% | 26% | 27% | | | |
| | COVERAGE | | 96% | 105% | | | | 100% | 89% | 97% | | | |
| SAU102476 | SeqID | | | | | | | 12175 | | | | | |
| | IDENTITY | | | | | | | 100% | | | | | |
| | COVERAGE | | | | | | | 100% | | | | | |
| SAU102479 | SeqID | 10306 | | | | | | 12405 | | | | | |
| | IDENTITY | 26% | | | | | | 100% | | | | | |
| | COVERAGE | 84% | | | | | | 100% | | | | | |
| SAU102480 | SeqID | 10310 | 10935 | | | | 11871 | 12404 | | 13770 | | | |
| | IDENTITY | 28% | 33% | | | | 30% | 100% | | 27% | | | |
| | COVERAGE | 100% | 88% | | | | 100% | 100% | | 100% | | | |
| SAU102481 | SeqID | 10289 | 10831 | | | | | 12422 | | 13879 | | | |
| | IDENTITY | 26% | 29% | | | | | 100% | | 26% | | | |
| | COVERAGE | 102% | 94% | | | | | 101% | | 102% | | | |
| SAU102485 | SeqID | 10457 | 10890 | | | | | 12421 | 13512 | 13961 | | | |
| | IDENTITY | 28% | 53% | | | | | 100% | 56% | 60% | | | |
| | COVERAGE | 86% | 100% | | | | | 100% | 99% | 93% | | | |
| SAU102486 | SeqID | 10294 | 10889 | 11025 | | | | 12420 | 13513 | 13962 | | | |
| | IDENTITY | 36% | 38% | 27% | | | | 100% | 42% | 37% | | | |
| | COVERAGE | 95% | 97% | 95% | | | | 101% | 93% | 95% | | | |
| SAU102487 | SeqID | | | | | | | 12419 | | | | | |
| | IDENTITY | | | | | | | 100% | | | | | |
| | COVERAGE | | | | | | | 100% | | | | | |
| SAU102498 | SeqID | 10241 | 10597 | 10974 | 11342 | 11706 | 11842 | 12688 | 13387 | 14092 | | | |
| | IDENTITY | 36% | 35% | 35% | 33% | 37% | 38% | 100% | 35% | 36% | | | |
| | COVERAGE | 93% | 94% | 93% | 92% | 94% | 94% | 100% | 93% | 93% | | | |
| SAU102502 | SeqID | | | | | | 12060 | 12689 | | | | | |
| | IDENTITY | | | | | | 26% | 100% | | | | | |
| | COVERAGE | | | | | | 85% | 100% | | | | | |
| SAU102503 | SeqID | | | | | | 12059 | 12690 | | | | | |
| | IDENTITY | | | | | | 32% | 100% | | | | | |
| | COVERAGE | | | | | | 92% | 100% | | | | | |
| SAU102526 | SeqID | | | | | | | 12691 | | | | | |
| | IDENTITY | | | | | | | 100% | | | | | |
| | COVERAGE | | | | | | | 100% | | | | | |
| SAU102527 | SeqID | 10352 | 10560 | 11104 | 11439 | | 5171 | 12260 | 13204 | 13968 | | | |
| | IDENTITY | 54% | 74% | 55% | 56% | | 58% | 100% | 75% | 54% | | | |
| | COVERAGE | 93% | 101% | 93% | 94% | | 93% | 101% | 94% | 93% | | | |
| SAU102531 | SeqID | | | | | | 10765 | 12667 | | | | | |
| | IDENTITY | | 34% | | | | | 100% | | | | | |
| | COVERAGE | | 102% | | | | | 100% | | | | | |
| SAU102541 | SeqID | 10076 | 10520 | 11000 | 11498 | | 11966 | 12668 | 13405 | 13718 | | | |
| | IDENTITY | 41% | 49% | 38% | 37% | | 44% | 100% | 45% | 41% | | | |
| | COVERAGE | 93% | 102% | 91% | 93% | | 100% | 100% | 81% | 93% | | | |
| SAU102551 | SeqID | | | 11013 | 11353 | | 11816 | 12672 | 13271 | | | | |
| | IDENTITY | | | 47% | 38% | | 39% | 100% | 41% | | | | |
| | COVERAGE | | | 87% | 84% | | 84% | 101% | 95% | | | | |
| SAU102554 | SeqID | | 10494 | | | | | 12673 | 13466 | | | | |
| | IDENTITY | | 47% | | | | | 100% | 44% | | | | |
| | COVERAGE | | 99% | | | | | 100% | 98% | | | | |
| SAU102575 | SeqID | 10166 | | 11232 | 11618 | | 11777 | 12609 | | 13836 | | | |
| | IDENTITY | 28% | | 29% | 35% | | 30% | 100% | | 27% | | | |
| | COVERAGE | 98% | | 91% | 99% | | 96% | 100% | | 98% | | | |
| SAU102578 | SeqID | 1045910948 | 11050 | 11420 | | 12074 | 12411 | 13503 | 13859 | | | | |
| | IDENTITY | 59% | 76% | 60% | 51% | | 65% | 100% | 73% | 59% | | | |
| | COVERAGE | 88% | 95% | 88% | 89% | | 81% | 101% | 94% | 89% | | | |
| SAU102584 | SeqID | | | | | | | 12537 | | | | | |
| | IDENTITY | | | | | | | 100% | | | | | |
| | COVERAGE | | | | | | | 100% | | | | | |
| SAU102585 | SeqID | | | | | | | 12611 | | | | | |
| | IDENTITY | | | | | | | 100% | | | | | |
| | COVERAGE | | | | | | | 100% | | | | | |
| SAU102593 | SeqID | | 10889 | | | | | 12463 | 13513 | | | | |
| | IDENTITY | | 27% | | | | | 100% | 27% | | | | |
| | COVERAGE | | 87% | | | | | 100% | 88% | | | | |
| SAU102598 | SeqID | 10187 | | 10958 | | 11710 | 11979 | 12464 | | 13833 | | | |
| | IDENTITY | 30% | | 32% | | 27% | 31% | 100% | | 31% | | | |
| | COVERAGE | 102% | | 85% | | 75% | 92% | 100% | | 86% | | | |
| SAU102599 | SeqID | 10206 | 10944 | 10958 | 11619 | | 11975 | 12466 | 13653 | 13773 | | | |
| | IDENTITY | 36% | 26% | 30% | 30% | | 33% | 100% | 32% | 32% | | | |
| | COVERAGE | 89% | 76% | 93% | 73% | | 79% | 100% | 77% | 101% | | | |
| SAU102601 | SeqID | 10273 | | 11076 | | 11722 | 11931 | 12467 | 13256 | 13867 | | | |
| | IDENTITY | 27% | | 30% | | 28% | 28% | 100% | 51% | 27% | | | |
| | COVERAGE | 95% | | 93% | | 95% | 93% | 100% | 97% | 92% | | | |
| SAU102602 | SeqID | 10356 | 10555 | 11100 | 11441 | 11679 | 11993 | 12249 | 13200 | 13971 | | | |
| | IDENTITY | 58% | 78% | 61% | 57% | 59% | 60% | 100% | 77% | 58% | | | |
| | COVERAGE | 100% | 100% | 100% | 100% | 100% | 99% | 100% | 100% | 99% | | | |
| SAU102603 | SeqID | | | | | | | 12469 | | | | | |
| | IDENTITY | | | | | | | 100% | | | | | |
| | COVERAGE | | | | | | | 100% | | | | | |
| SAU102605 | SeqID | | 10836 | | | | | 12470 | | | | | |
| | IDENTITY | | 47% | | | | | 100% | | | | | |
| | COVERAGE | | 96% | | | | | 100% | | | | | |
| SAU102606 | SeqID | 10273 | | 11076 | | 11722 | 11931 | 12471 | 13256 | 13867 | | | |
| | IDENTITY | 27% | | 30% | | 27% | 25% | 100% | 50% | 26% | | | |
| | COVERAGE | 95% | | 92% | | 95% | 93% | 100% | 97% | 94% | | | |
| SAU102607 | SeqID | | | | | | | 12472 | 13579 | | | | |
| | IDENTITY | | | | | | | 100% | 43% | | | | |
| | COVERAGE | | | | | | | 100% | 98% | | | | |
| SAU102609 | SeqID | | | | | | | 12473 | | | | | |
| | IDENTITY | | | | | | | 100% | | | | | |
| | COVERAGE | | | | | | | 100% | | | | | |
| SAU102610 | SeqID | | | | | | | 12474 | | | | | |
| | IDENTITY | | | | | | | 100% | | | | | |
| | COVERAGE | | | | | | | 100% | | | | | |
| SAU102613 | SeqID | 10461 | | 11272 | | | | 12475 | | 13988 | | | |
| | IDENTITY | 26% | | 28% | | | | 100% | | 26% | | | |
| | COVERAGE | 97% | | 95% | | | | 100% | | 97% | | | |
| SAU102614 | SeqID | 10211 | 10600 | | | | | 12476 | | 13927 | | | |
| | IDENTITY | 33% | 55% | | | | | 100% | | 32% | | | |
| | COVERAGE | 89% | 100% | | | | | 100% | | 89% | | | |
| SAU102615 | SeqID | 10234 | 10601 | | | 11720 | 12098 | 12477 | | 13926 | | | |
| | IDENTITY | 32% | 40% | | | 32% | 26% | 100% | | 31% | | | |
| | COVERAGE | 98% | 100% | | | 92% | 87% | 100% | | 100% | | | |
| SAU102620 | SeqID | | | | | | | 12479 | | | | | |
| | IDENTITY | | | | | | | 100% | | | | | |
| | COVERAGE | | | | | | | 100% | | | | | |
| SAU102621 | SeqID | 10288 | 10519 | | | 11724 | | 12480 | 13370 | 13881 | | | |
| | IDENTITY | 61% | 62% | | | 58% | | 100% | 59% | 61% | | | |
| | COVERAGE | 100% | 101% | | | 81% | | 100% | 101% | 100% | | | |
| SAU102629 | SeqID | | 10885 | | | | | 12481 | | | | | |
| | IDENTITY | | 26% | | | | | 100% | | | | | |
| | COVERAGE | | 108% | | | | | 100% | | | | | |
| SAU102631 | SeqID | | 10522 | | | 11657 | 11841 | 12712 | | | | | |
| | IDENTITY | | 27% | | | 44% | 32% | 100% | | | | | |
| | COVERAGE | | 83% | | | 83% | 81% | 100% | | | | | |
| SAU102636 | SeqID | | | | | | | 12650 | 13696 | | | | |
| | IDENTITY | | | | | | | 100% | 29% | | | | |
| | COVERAGE | | | | | | | 100% | 102% | | | | |
| SAU102637 | SeqID | | | | | | | 12651 | 13697 | | | | |
| | IDENTITY | | | | | | | 100% | 39% | | | | |
| | COVERAGE | | | | | | | 100% | 98% | | | | |
| SAU102652 | SeqID | | | | | | | 12653 | | | | | |
| | IDENTITY | | | | | | | 100% | | | | | |
| | COVERAGE | | | | | | | 101% | | | | | |
| SAU102658 | SeqID | 10283 | 10910 | 11064 | | | 12090 | 12654 | 13514 | 13855 | | | |
| | IDENTITY | 45% | 54% | 42% | | | 39% | 100% | 49% | 41% | | | |
| | COVERAGE | 97% | 92% | 97% | | | 97% | 100% | 96% | 100% | | | |
| SAU102663 | SeqID | 10304 | 10840 | 11043 | 11626 | | 11798 | 12158 | 13172 | 13780 | | | |
| | IDENTITY | 43% | 58% | 44% | 34% | | 45% | 100% | 56% | 41% | | | |
| | COVERAGE | 99% | 99% | 96% | 95% | | 91% | 100% | 97% | 99% | | | |
| SAU102669 | SeqID | 10022 | 10756 | 11257 | | | 12045 | 12160 | 13371 | 14035 | | | |
| | IDENTITY | 42% | 26% | 43% | | | 41% | 100% | 54% | 41% | | | |
| | COVERAGE | 96% | 91% | 95% | | | 94% | 100% | 95% | 93% | | | |
| SAU102671 | SeqID | 10409 | | 11079 | 11319 | 11683 | 12043 | 12161 | 13373 | 14033 | | | |
| | IDENTITY | 34% | | 32% | 44% | 35% | 56% | 100% | 69% | 33% | | | |
| | COVERAGE | 91% | | 91% | 96% | 74% | 99% | 100% | 96% | 91% | | | |
| SAU102674 | SeqID | 10020 | | 11164 | | 11648 | 5127 | 12156 | | 14016 | | | |
| | IDENTITY | 55% | | 54% | | 46% | 55% | 100% | | 53% | | | |
| | COVERAGE | 102% | | 103% | | 101% | 105% | 101% | | 102% | | | |
| SAU102693 | SeqID | 10178 | 10659 | | 11474 | | 11883 | 12627 | 13301 | 13940 | | | |
| | IDENTITY | 53% | 74% | | 38% | | 49% | 100% | 61% | 49% | | | |
| | COVERAGE | 82% | 87% | | 86% | | 86% | 101% | 90% | 72% | | | |
| SAU102694 | SeqID | 10177 | 10660 | 11222 | 11296 | | 5120 | 12628 | 13302 | | | | |
| | IDENTITY | 48% | 66% | 50% | 44% | | 55% | 100% | 60% | | | | |
| | COVERAGE | 97% | 102% | 97% | 94% | | 94% | 102% | 102% | | | | |
| SAU102725 | SeqID | 10418 | 10514 | 11137 | 11507 | | 12088 | 12338 | 13378 | 13789 | | | |
| | IDENTITY | 40% | 72% | 39% | 38% | | 37% | 100% | 66% | 40% | | | |
| | COVERAGE | 96% | 100% | 96% | 103% | | 104% | 100% | 100% | 96% | | | |
| SAU102764 | SeqID | 10179 | 10929 | 11234 | 11295 | | 11884 | 12625 | 13484 | 13938 | | | |
| | IDENTITY | 44% | 67% | 42% | 41% | | 42% | 100% | 63% | 43% | | | |
| | COVERAGE | 99% | 99% | 99% | 90% | | 97% | 100% | 99% | 99% | | | |
| SAU102812 | SeqID | | 10860 | | | | 12127 | 13253 | | | | | |
| | IDENTITY | | 48% | | | | 100% | 49% | | | | | |
| | COVERAGE | | 100% | | | | 101% | 96% | | | | | |
| SAU102870 | SeqID | 10113 | 10880 | | | | | 12170 | 13270 | 14008 | | | |
| | IDENTITY | 29% | 35% | | | | | 100% | 29% | 28% | | | |
| | COVERAGE | 92% | 83% | | | | | 100% | 93% | 87% | | | |
| SAU102880 | SeqID | 10360 | 10533 | 11096 | 11443 | 11643 | 5177 | 12224 | 13196 | 13975 | | | |
| | IDENTITY | 60% | 82% | 61% | 57% | 61% | 58% | 100% | 85% | 61% | | | |
| | COVERAGE | 100% | 101% | 100% | 97% | 100% | 100% | 101% | 101% | 100% | | | |
| SAU102881 | SeqID | 10357 | 10551 | 11099 | | | 11994 | 12242 | 13199 | 13972 | | | |
| | IDENTITY | 38% | 69% | 37% | | | 38% | 100% | 54% | 38% | | | |
| | COVERAGE | 89% | 98% | 89% | | | 89% | 101% | 102% | 89% | | | |
| SAU102883 | SeqID | 10396 | | 11168 | 11449 | | 12118 | 12702 | 13181 | | | | |
| | IDENTITY | 63% | | 70% | 60% | | 65% | 100% | 76% | | | | |
| | COVERAGE | 86% | | 88% | 86% | | 86% | 102% | 90% | | | | |
| SAU102905 | SeqID | | 10732 | 11217 | 11373 | | | 12273 | | | | | |
| | IDENTITY | | 31% | 26% | 38% | | | 100% | | | | | |
| | COVERAGE | | 92% | 80% | 87% | | | 100% | | | | | |
| SAU102909 | SeqID | 10042 | 10488 | 11150 | 11457 | 11637 | 11940 | 12315 | 13437 | 13908 | | | |
| | IDENTITY | 59% | 68% | 60% | 69% | 59% | 60% | 100% | 73% | 59% | | | |
| | COVERAGE | 95% | 95% | 95% | 130% | 95% | 98% | 101% | 124% | 95% | | | |
| SAU102933 | SeqID | 10448 | 10949 | 10995 | 11579 | 11762 | 11985 | 12412 | 13502 | 13817 | | | |
| | IDENTITY | 33% | 53% | 35% | 32% | 31% | 29% | 100% | 50% | 31% | | | |
| | COVERAGE | 104% | 113% | 101% | 108% | 107% | 101% | 101% | 101% | 103% | | | |
| SAU102936 | SeqID | 10236 | 10872 | | | | 11804 | 12356 | | 13955 | | | |
| | IDENTITY | 33% | 66% | | | | 60% | 100% | | 33% | | | |
| | COVERAGE | 97% | 100% | | | | 96% | 101% | | 98% | | | |
| SAU102942 | SeqID | 10136 | 10492 | 11230 | | 11696 | | 12296 | 13339 | 13834 | | | |
| | IDENTITY | 52% | 55% | 43% | | 50% | | 100% | 51% | 51% | | | |
| | COVERAGE | 100% | 100% | 99% | | 99% | | 100% | 99% | 99% | | | |
| SAU102944 | SeqID | | | | | | | 12468 | 13257 | | | | |
| | IDENTITY | | | | | | | 100% | 42% | | | | |
| | COVERAGE | | | | | | | 100% | 99% | | | | |
| SAU102979 | SeqID | 10014 | | 10979 | 11384 | | 11936 | 12536 | 13429 | 13712 | | | |
| | IDENTITY | 33% | | 37% | 32% | | 41% | 100% | 33% | 33% | | | |
| | COVERAGE | 88% | | 87% | 87% | | 87% | 100% | 87% | 90% | | | |
| SAU102983 | SeqID | | 10883 | | | | | 12676 | 13269 | | | | |
| | IDENTITY | | 28% | | | | | 100% | 27% | | | | |
| | COVERAGE | | 70% | | | | | 100% | 76% | | | | |
| SAU102992 | SeqID | 10176 | 10661 | 11223 | 11297 | | 11882 | 12630 | 13303 | 13941 | | | |
| | IDENTITY | 62% | 70% | 62% | 48% | | 59% | 100% | 63% | 61% | | | |
| | COVERAGE | 99% | 92% | 99% | 97% | | 99% | 101% | 99% | 101% | | | |
| SAU103010 | SeqID | | | | | | | 12194 | | | | | |
| | IDENTITY | | | | | | | 100% | | | | | |
| | COVERAGE | | | | | | | 100% | | | | | |
| SAU103024 | SeqID | | | | | 11670 | 12042 | 12200 | | | | | |
| | IDENTITY | | | | | 44% | 26% | 100% | | | | | |
| | COVERAGE | | | | | 89% | 72% | 101% | | | | | |
| SAU103025 | SeqID | | | | | | | 12202 | | | | | |
| | IDENTITY | | | | | | | 100% | | | | | |
| | COVERAGE | | | | | | | 100% | | | | | |
| SAU103037 | SeqID | | 10867 | | | | | 12613 | 13267 | | | | |
| | IDENTITY | | 27% | | | | | 100% | 26% | | | | |
| | COVERAGE | | 99% | | | | | 101% | 86% | | | | |
| SAU103077 | SeqID | | | | | | | 12408 | | | | | |
| | IDENTITY | | | | | | | 100% | | | | | |
| | COVERAGE | | | | | | | 100% | | | | | |
| SAU103115 | SeqID | | | | | | | 12508 | 13469 | | | | |
| | IDENTITY | | | | | | | 100% | 32% | | | | |
| | COVERAGE | | | | | | | 101% | 101% | | | | |
| SAU103 | 144 | SeqID | | 10936 | | | | | 12663 | | | | |
| | IDENTITY | | 42% | | | | | 100% | | | | | |
| | COVERAGE | | 84% | | | | | 100% | | | | | |
| SAU103159 | SeqID | 10110 | 10783 | 11134 | 11489 | | 11787 | 12670 | 13411 | 13994 | | | |
| | IDENTITY | 43% | 48% | 38% | 48% | | 48% | 100% | 63% | 43% | | | |
| | COVERAGE | 115% | 100% | 112% | 117% | | 98% | 100% | 101% | 116% | | | |
| SAU103169 | SeqID | | | | | | | 12678 | 13239 | | | | |
| | IDENTITY | | | | | | | 100% | 34% | | | | |
| | COVERAGE | | | | | | | 100% | 84% | | | | |
| SAU103175 | SeqID | 10157 | | | | | | 12687 | | | | | |
| | IDENTITY | 36% | | | | | | 100% | | | | | |
| | COVERAGE | 96% | | | | | | 100% | | | | | |
| SAU103191 | SeqID | | | | | | | 12465 | 13332 | | | | |
| | IDENTITY | | | | | | | 100% | 42% | | | | |
| | COVERAGE | | | | | | | 102% | 75% | | | | |
| SAU103204 | SeqID | | | | | | | 12499 | | | | | |
| | IDENTITY | | | | | | | 100% | | | | | |
| | COVERAGE | | | | | | | 101% | | | | | |
| SAU103226 | SeqID | | | | | | | 12713 | | | | | |
| | IDENTITY | | | | | | | 100% | | | | | |
| | COVERAGE | | | | | | | 100% | | | | | |
| SAU103232 | SeqID | 10368 | | | | 11704 | 11848 | 12697 | | 13803 | | | |
| | IDENTITY | 36% | | | | 35% | 48% | 100% | | 35% | | | |
| | COVERAGE | 102% | | | | 98% | 101% | 101% | | 102% | | | |
| SAU200006 | SeqID | 10033 | 10639 | 11192 | 11553 | | 12007 | 12723 | 13479 | | | | |
| | IDENTITY | 53% | 70% | 47% | 43% | | 50% | 100% | 65% | | | | |
| | COVERAGE | 78% | 80% | 84% | 82% | | 89% | 100% | 77% | | | | |
| SAU200028 | SeqID | | | | | | | 12694 | | | | | |
| | IDENTITY | | | | | | | 100% | | | | | |
| | COVERAGE | | | | | | | 100% | | | | | |
| SAU200030 | SeqID | 10372 | 10553 | 11056 | 11447 | 11672 | 12092 | 12745 | 13449 | 13807 | | | |
| | IDENTITY | 42% | 74% | 39% | 43% | 41% | 35% | 100% | 73% | 42% | | | |
| | COVERAGE | 84% | 98% | 84% | 93% | 86% | 93% | 102% | 95% | 84% | | | |
| SAU200058 | SeqID | | 10621 | | | | | 12719 | 13327 | | | | |
| | IDENTITY | | 39% | | | | | 100% | 37% | | | | |
| | COVERAGE | | 79% | | | | | 101% | 78% | | | | |
| SAU200059 | SeqID | 10259 | 10622 | 10978 | | | 12026 | 12720 | 13325 | 14087 | | | |
| | IDENTITY | 31% | 33% | 32% | | | 36% | 100% | 40% | 31% | | | |
| | COVERAGE | 73% | 97% | 73% | | | 74% | 100% | 96% | 73% | | | |
| SAU200088 | SeqID | 10262 | | 10984 | 11403 | | 11947 | 12724 | 13415 | 14090 | | | |
| | IDENTITY | 51% | | 56% | 57% | | 45% | 100% | 68% | 49% | | | |
| | COVERAGE | 82% | | 91% | 93% | | 93% | 102% | 100% | 82% | | | |
| SAU200242 | SeqID | | 10712 | | | | | 12734 | | | | | |
| | IDENTITY | | 28% | | | | | 100% | | | | | |
| | COVERAGE | | 99% | | | | | 100% | | | | | |
| SAU200297 | SeqID | 10109 | 10756 | 11257 | | | 11982 | 12739 | 13371 | 13996 | | | |
| | IDENTITY | 33% | 64% | 34% | | | 33% | 100% | 33% | 32% | | | |
| | COVERAGE | 95% | 100% | 98% | | | 95% | 100% | 95% | 95% | | | |
| SAU200345 | SeqID | | | | | | | 12751 | | | | | |
| | IDENTITY | | | | | | | 100% | | | | | |
| | COVERAGE | | | | | | | 100% | | | | | |
| SAU200392 | SeqID | 10164 | 10584 | 10968 | 11566 | | 11912 | 12755 | | 13892 | | | |
| | IDENTITY | 26% | 30% | 25% | 27% | | 33% | 100% | | 26% | | | |
| | COVERAGE | 97% | 80% | 96% | 98% | | 93% | 100% | | 98% | | | |
| SAU200468 | SeqID | 10201 | 10478 | 11054 | | 12061 | 12937 | 13425 | 13822 | | | | |
| | IDENTITY | 78% | 75% | 62% | | 36% | 100% | 76% | 78% | | | | |
| | COVERAGE | 74% | 75% | 74% | | 81% | 101% | 75% | 74% | | | | |
| SAU200558 | SeqID | 10039 | 10728 | 11277 | | | 12046 | 12777 | 13423 | 13904 | | | |
| | IDENTITY | 28% | 31% | 26% | | | 30% | 100% | 32% | 29% | | | |
| | COVERAGE | 72% | 102% | 80% | | | 75% | 100% | 99% | 72% | | | |
| SAU200561 | SeqID | | | | | | | 12693 | | | | | |
| | IDENTITY | | | | | | | 100% | | | | | |
| | COVERAGE | | | | | | | 100% | | | | | |
| SAU200564 | SeqID | 10099 | | 11170 | 11602 | 11645 | 11788 | 12780 | | 13992 | | | |
| | IDENTITY | 33% | | 31% | 31% | 34% | 32% | 100% | | 34% | | | |
| | COVERAGE | 87% | | 87% | 82% | 86% | 93% | 100% | | 87% | | | |
| SAU200565 | SeqID | 10098 | | 11250 | 11386 | | 11786 | 12781 | | 13991 | | | |
| | IDENTITY | 32% | | 34% | 35% | | 39% | 100% | | 33% | | | |
| | COVERAGE | 97% | | 96% | 98% | | 97% | 100% | | 97% | | | |
| SAU200593 | SeqID | 10435 | 10613 | 11038 | 11412 | | 11998 | 12784 | 13397 | 14046 | | | |
| | IDENTITY | 53% | 73% | 50% | 53% | | 52% | 100% | 64% | 52% | | | |
| | COVERAGE | 99% | 100% | 99% | 98% | | 100% | 100% | 99% | 99% | | | |
| SAU200628 | SeqID | 10173 | 10856 | | | | | 12790 | 13297 | 13937 | | | |
| | IDENTITY | 32% | 31% | | | | | 100% | 29% | 34% | | | |
| | COVERAGE | 92% | 97% | | | | | 100% | 97% | 94% | | | |
| SAU200685 | SeqID | | | | | | | 12801 | 13185 | | | | |
| | IDENTITY | | | | | | | 100% | 31% | | | | |
| | COVERAGE | | | | | | | 100% | 94% | | | | |
| SAU200721 | SeqID | 10208 | 10582 | 11015 | 11541 | | | 12797 | 13681 | 13922 | | | |
| | IDENTITY | 40% | 33% | 41% | 36% | | | 100% | 42% | 41% | | | |
| | COVERAGE | 92% | 79% | 99% | 94% | | | 100% | 100% | 94% | | | |
| SAU200725 | SeqID | 10118 | 10761 | 10966 | | | 11780 | 12933 | 13632 | 14020 | | | |
| | IDENTITY | 30% | 46% | 30% | | | 25% | 100% | 47% | 29% | | | |
| | COVERAGE | 98% | 100% | 97% | | | 98% | 100% | 100% | 98% | | | |
| SAU200731 | SeqID | 10283 | 10822 | 11064 | | | 12090 | 12342 | 13514 | 13855 | | | |
| | IDENTITY | 55% | 54% | 44% | | | 43% | 100% | 51% | 46% | | | |
| | COVERAGE | 99% | 100% | 98% | | | 98% | 100% | 100% | 99% | | | |
| SAU200740 | SeqID | 10318 | 10554 | 11225 | 11393 | | 12056 | 12798 | 13695 | 13760 | | | |
| | IDENTITY | 48% | 56% | 48% | 49% | | 50% | 100% | 55% | 48% | | | |
| | COVERAGE | 86% | 102% | 86% | 73% | | 87% | 100% | 93% | 86% | | | |
| SAU200752 | SeqID | | | | | | | 12809 | | | | | |
| | IDENTITY | | | | | | | 100% | | | | | |
| | COVERAGE | | | | | | | 100% | | | | | |
| SAU200914 | SeqID | 10383 | 10714 | | | 11747 | 11927 | 12837 | 13431 | 13788 | | | |
| | IDENTITY | 26% | 28% | | | 27% | 27% | 100% | 25% | 25% | | | |
| | COVERAGE | 96% | 98% | | | 79% | 90% | 100% | 91% | 90% | | | |
| SAU200916 | SeqID | | | | | | | 12838 | | | | | |
| | IDENTITY | | | | | | | 100% | | | | | |
| | COVERAGE | | | | | | | 100% | | | | | |
| SAU200928 | SeqID | 10439 | 10627 | 11036 | 11571 | | 5179 | 12815 | 13646 | 14042 | | | |
| | IDENTITY | 54% | 73% | 55% | 53% | | 49% | 100% | 69% | 54% | | | |
| | COVERAGE | 86% | 99% | 87% | 86% | | 102% | 100% | 100% | 86% | | | |
| SAU200934 | SeqID | 10212 | 10780 | | | | 11964 | 12842 | | 13835 | | | |
| | IDENTITY | 44% | 60% | | | | 42% | 100% | | 42% | | | |
| | COVERAGE | 72% | 93% | | | | 82% | 100% | | 88% | | | |
| SAU200949 | SeqID | | | | | | | 12846 | | | | | |
| | IDENTITY | | | | | | | 100% | | | | | |
| | COVERAGE | | | | | | | 100% | | | | | |
| SAU200960 | SeqID | | | | 11500 | | 11886 | 12431 | | | | | |
| | IDENTITY | | | | 42% | | 33% | 100% | | | | | |
| | COVERAGE | | | | 70% | | 91% | 102% | | | | | |
| SAU200994 | SeqID | 10036 | 10497 | 11270 | | | 11865 | 12935 | 13310 | 14054 | | | |
| | IDENTITY | 36% | 62% | 32% | | | 37% | 100% | 35% | 33% | | | |
| | COVERAGE | 100% | 101% | 100% | | | 102% | 100% | 73% | 99% | | | |
| SAU201 | 167 | SeqID | | 10779 | | | | | 12887 | | | | |
| | IDENTITY | | 37% | | | | | 100% | | | | | |
| | COVERAGE | | 98% | | | | | 100% | | | | | |
| SAU201168 | SeqID | | 10819 | | | | | 12889 | 13626 | | | | |
| | IDENTITY | | 53% | | | | | 100% | 56% | | | | |
| | COVERAGE | | 102% | | | | | 100% | 100% | | | | |
| SAU201184 | SeqID | 10448 | 10715 | 10995 | 11579 | | 11985 | 12807 | 13502 | 13819 | | | |
| | IDENTITY | 40% | 52% | 35% | 37% | | 37% | 100% | 53% | 32% | | | |
| | COVERAGE | 70% | 108% | 97% | 82% | | 70% | 101% | 111% | 111% | | | |
| SAU201197 | SeqID | 10330 | 10924 | 11160 | 11321 | | 5215 | 12938 | 13364 | 13885 | | | |
| | IDENTITY | 58% | 66% | 60% | 53% | | 58% | 100% | 63% | 58% | | | |
| | COVERAGE | 99% | 99% | 99% | 98% | | 99% | 101% | 96% | 99% | | | |
| SAU201225 | SeqID | | 10812 | 11090 | | | | 12896 | 13170 | | | | |
| | IDENTITY | | 41% | 33% | | | | 100% | 38% | | | | |
| | COVERAGE | | 93% | 80% | | | | 100% | 87% | | | | |
| SAU201236 | SeqID | 10026 | 10679 | 11184 | 11613 | | 12013 | 12891 | 13505 | 14073 | | | |
| | IDENTITY | 32% | 29% | 33% | 33% | | 34% | 100% | 30% | 32% | | | |
| | COVERAGE | 92% | 96% | 93% | 89% | | 95% | 100% | 95% | 90% | | | |
| SAU201301 | SeqID | | | | | | | 12899 | | | | | |
| | IDENTITY | | | | | | | 100% | | | | | |
| | COVERAGE | | | | | | | 100% | | | | | |
| SAU201333 | SeqID | 10192 | | | | 11678 | | 12905 | | 13753 | | | |
| | IDENTITY | 41% | | | | 28% | | 100% | | 41% | | | |
| | COVERAGE | 100% | | | | 96% | | 101% | | 100% | | | |
| SAU201375 | SeqID | | | | | | 11929 | 12926 | | | | | |
| | IDENTITY | | | | | | 36% | 100% | | | | | |
| | COVERAGE | | | | | | 95% | 100% | | | | | |
| SAU201380 | SeqID | 10379 | 10499 | | 11313 | | 12024 | 12922 | | 13801 | | | |
| | IDENTITY | 34% | 25% | | 26% | | 25% | 100% | | 25% | | | |
| | COVERAGE | 94% | 93% | | 95% | | 89% | 100% | | 101% | | | |
| SAU201381 | SeqID | 10241 | 10597 | 10974 | 11387 | 11706 | 11833 | 12923 | 13387 | 13878 | | | |
| | IDENTITY | 68% | 59% | 46% | 44% | 56% | 57% | 100% | 52% | 64% | | | |
| | COVERAGE | 89% | 96% | 90% | 91% | 89% | 100% | 104% | 92% | 89% | | | |
| SAU201403 | SeqID | | | | | | | 12913 | | | | | |
| | IDENTITY | | | | | | | 100% | | | | | |
| | COVERAGE | | | | | | | 100% | | | | | |
| SAU201469 | SeqID | | | | | | | 12967 | | | | | |
| | IDENTITY | | | | | | | 100% | | | | | |
| | COVERAGE | | | | | | | 100% | | | | | |
| SAU201486 | SeqID | | | | | | | 13023 | | | | | |
| | IDENTITY | | | | | | | 100% | | | | | |
| | COVERAGE | | | | | | | 100% | | | | | |
| SAU201506 | SeqID | 10145 | | | | | 11963 | 12946 | | 13841 | | | |
| | IDENTITY | 49% | | | | | 49% | 100% | | 50% | | | |
| | COVERAGE | 101% | | | | | 102% | 100% | | 100% | | | |
| SAU201508 | SeqID | 10370 | | | | | 11874 | 12947 | | 13805 | | | |
| | IDENTITY | 37% | | | | | 42% | 100% | | 36% | | | |
| | COVERAGE | 73% | | | | | 72% | 100% | | 73% | | | |
| SAU201513 | SeqID | 10229 | | | | | | 12944 | | | | | |
| | IDENTITY | 29% | | | | | | 100% | | | | | |
| | COVERAGE | 71% | | | | | | 101% | | | | | |
| SAU201539 | SeqID | 10109 | | 11257 | | | 5099 | 12943 | 13625 | 13996 | | | |
| | IDENTITY | 33% | | 28% | | | 34% | 100% | 32% | 33% | | | |
| | COVERAGE | 95% | | 96% | | | 96% | 100% | 97% | 95% | | | |
| SAU201541 | SeqID | 10131 | 10500 | | | 11673 | 11951 | 12942 | 13474 | | | | |
| | IDENTITY | 50% | 39% | | | 33% | 41% | 100% | 41% | | | | |
| | COVERAGE | 71% | 74% | | | 77% | 73% | 100% | 73% | | | | |
| SAU201558 | SeqID | 10112 | | 11258 | 11396 | | 11875 | 12954 | 13598 | 14009 | | | |
| | IDENTITY | 51% | | 51% | 43% | | 49% | 100% | 46% | 51% | | | |
| | COVERAGE | 96% | | 94% | 94% | | 99% | 101% | 96% | 96% | | | |
| SAU201571 | SeqID | 10224 | 10951 | 11213 | 11357 | | 11905 | 12997 | 13268 | 13957 | | | |
| | IDENTITY | 50% | 61% | 47% | 50% | | 45% | 100% | 54% | 49% | | | |
| | COVERAGE | 98% | 94% | 99% | 92% | | 103% | 100% | 70% | 98% | | | |
| SAU201611 | SeqID | | | | 11539 | | 11902 | 12973 | 13243 | | | | |
| | IDENTITY | | | | 38% | | 48% | 100% | 58% | | | | |
| | COVERAGE | | | | 73% | | 99% | 100% | 95% | | | | |
| SAU201615 | SeqID | | | | | | 11962 | 12972 | | | | | |
| | IDENTITY | | | | | | 40% | 100% | | | | | |
| | COVERAGE | | | | | | 72% | 100% | | | | | |
| SAU201621 | SeqID | 10038 | 10842 | | 11392 | 11707 | 12047 | 12662 | | 13902 | | | |
| | IDENTITY | 49% | 53% | | 42% | 49% | 47% | 100% | | 46% | | | |
| | COVERAGE | 91% | 91% | | 91% | 91% | 91% | 101% | | 91% | | | |
| SAU201654 | SeqID | | | | | | | 12982 | | | | | |
| | IDENTITY | | | | | | | 100% | | | | | |
| | COVERAGE | | | | | | | 101% | | | | | |
| SAU201666 | SeqID | 10291 | 10900 | 11028 | 11557 | 11761 | 11811 | 12981 | | 13743 | | | |
| | IDENTITY | 33% | 29% | 35% | 31% | 32% | 34% | 100% | | 33% | | | |
| | COVERAGE | 71% | 80% | 71% | 76% | 79% | 73% | 100% | | 71% | | | |
| SAU201752 | SeqID | | 10623 | | | | | 12963 | 13689 | | | | |
| | IDENTITY | | 45% | | | | | 100% | 40% | | | | |
| | COVERAGE | | 89% | | | | | 100% | 92% | | | | |
| SAU201765 | SeqID | | | | | | | 12770 | | | | | |
| | IDENTITY | | | | | | | 100% | | | | | |
| | COVERAGE | | | | | | | 100% | | | | | |
| SAU20 | 1773 | SeqID | | | | | | | 12996 | | | | |
| | IDENTITY | | | | | | | 100% | | | | | |
| | COVERAGE | | | | | | | 100% | | | | | |
| SAU20 | 1775 | SeqID | | | | | | | 12996 | | | | |
| | IDENTITY | | | | | | | 100% | | | | | |
| | COVERAGE | | | | | | | 100% | | | | | |
| SAU201810 | SeqID | | | | | | | 12769 | | | | | |
| | IDENTITY | | | | | | | 100% | | | | | |
| | COVERAGE | | | | | | | 100% | | | | | |
| SAU201827 | SeqID | 10258 | 10783 | 11134 | 11310 | | 11787 | 13002 | 13411 | 14088 | | | |
| | IDENTITY | 38% | 46% | 41% | 41% | | 45% | 100% | 63% | 39% | | | |
| | COVERAGE | 108% | 100% | 100% | 104% | | 88% | 100% | 101% | 108% | | | |
| SAU201929 | SeqID | | | | | | | 13008 | | | | | |
| | IDENTITY | | | | | | | 100% | | | | | |
| | COVERAGE | | | | | | | 100% | | | | | |
| SAU20 | 1952 | SeqID | | | | | | | 13020 | | | | |
| | IDENTITY | | | | | | | 100% | | | | | |
| | COVERAGE | | | | | | | 100% | | | | | |
| SAU201971 | SeqID | | | | | | | 13015 | | | | | |
| | IDENTITY | | | | | | | 100% | | | | | |
| | COVERAGE | | | | | | | 101% | | | | | |
| SAU202006 | SeqID | | | | | | | 13018 | | | | | |
| | IDENTITY | | | | | | | 100% | | | | | |
| | COVERAGE | | | | | | | 100% | | | | | |
| SAU202039 | SeqID | | | | 11359 | | | 13009 | 13374 | | | | |
| | IDENTITY | | | | 44% | | | 100% | 48% | | | | |
| | COVERAGE | | | | 96% | | | 101% | 98% | | | | |
| SAU202126 | SeqID | 10261 | 10874 | 10983 | 11561 | | 11946 | 12714 | 13417 | 14085 | | | |
| | IDENTITY | 51% | 50% | 52% | 33% | | 46% | 100% | 58% | 52% | | | |
| | COVERAGE | 94% | 94% | 91% | 84% | | 93% | 101% | 94% | 94% | | | |
| SAU202174 | SeqID | | | | | | | 12895 | | | | | |
| | IDENTITY | | | | | | | 100% | | | | | |
| | COVERAGE | | | | | | | 101% | | | | | |
| SAU202 | 176 | SeqID | | | | | | | 12895 | | | | |
| | IDENTITY | | | | | | | 100% | | | | | |
| | COVERAGE | | | | | | | 101% | | | | | |
| SAU202186 | SeqID | 10062 | | | | | | 12731 | | | | | |
| | IDENTITY | 28% | | | | | | 100% | | | | | |
| | COVERAGE | 73% | | | | | | 101% | | | | | |
| SAU202267 | SeqID | | | | | | | 12727 | | | | | |
| | IDENTITY | | | | | | | 100% | | | | | |
| | COVERAGE | | | | | | | 100% | | | | | |
| SAU202708 | SeqID | 10428 | 10913 | | | | | 12855 | | 13735 | | | |
| | IDENTITY | 25% | 28% | | | | | 100% | | 25% | | | |
| | COVERAGE | 86% | 84% | | | | | 100% | | 86% | | | |
| SAU202736 | SeqID | 10148 | 10902 | 11181 | 11494 | 11677 | 11857 | 12927 | 13248 | 13844 | | | |
| | IDENTITY | 39% | 40% | 37% | 40% | 37% | 38% | 100% | 38% | 39% | | | |
| | COVERAGE | 95% | 93% | 98% | 91% | 80% | 93% | 100% | 103% | 95% | | | |
| SAU202756 | SeqID | 10436 | 10614 | 11071 | | | 5181 | 13027 | 13246 | 14045 | | | |
| | IDENTITY | 44% | 63% | 47% | | | 44% | 100% | 53% | 40% | | | |
| | COVERAGE | 97% | 92% | 86% | | | 92% | 100% | 91% | 97% | | | |
| SAU202781 | SeqID | | | | | | | 12718 | | | | | |
| | IDENTITY | | | | | | | 100% | | | | | |
| | COVERAGE | | | | | | | 100% | | | | | |
| SAU202872 | SeqID | | 10656 | | | | | 12866 | 13670 | | | | |
| | IDENTITY | | 45% | | | | | 100% | 28% | | | | |
| | COVERAGE | | 101% | | | | | 100% | 98% | | | | |
| SAU202882 | SeqID | | | | | | | 12848 | | | | | |
| | IDENTITY | | | | | | | 100% | | | | | |
| | COVERAGE | | | | | | | 101% | | | | | |
| SAU202930 | SeqID | | | | | | | 12871 | | | | | |
| | IDENTITY | | | | | | | 100% | | | | | |
| | COVERAGE | | | | | | | 100% | | | | | |
| SAU202945 | SeqID | | | | | | | 12868 | | | | | |
| | IDENTITY | | | | | | | 100% | | | | | |
| | COVERAGE | | | | | | | 100% | | | | | |
| SAU202968 | SeqID | | | | | | | 12886 | | | | | |
| | IDENTITY | | | | | | | 100% | | | | | |
| | COVERAGE | | | | | | | 100% | | | | | |
| SAU203001 | SeqID | | | | | | | 12894 | | | | | |
| | IDENTITY | | | | | | | 100% | | | | | |
| | COVERAGE | | | | | | | 100% | | | | | |
| SAU203007 | SeqID | | | | | | | 12893 | | | | | |
| | IDENTITY | | | | | | | 100% | | | | | |
| | COVERAGE | 100% | | | | | | | | | | | |
| SAU203196 | SeqID | | | | | | | 12945 | | | | | |
| | IDENTITY | | | | | | | 100% | | | | | |
| | COVERAGE | | | | | | | | 101% | | | | |
| SAU203293 | SeqID | | | | | | | 12979 | | | | | |
| | IDENTITY | | | | | | | 100% | | | | | |
| | COVERAGE | | | | | | | 101% | | | | | |
| SAU203296 | SeqID | | | | 11330 | | | 12263 | | | | | |
| | IDENTITY | | | | 29% | | | 100% | | | | | |
| | COVERAGE | | | | 88% | | | 101% | | | | | |
| SAU203524 | SeqID | | | | | | | 12957 | | | | | |
| | IDENTITY | | | | | | | 100% | | | | | |
| | COVERAGE | | | | | | | 100% | | | | | |
| SAU300110 | SeqID | 10054 | 10544 | | | 11662 | | 13031 | 13441 | | | | |
| | IDENTITY | 33% | 38% | | | 33% | | 100% | 30% | | | | |
| | COVERAGE | 82% | 109% | | | 73% | | 102% | 109% | | | | |
| SAU300131 | SeqID | 10344 | 10529 | 11112 | 11434 | | 5164 | 13034 | 13213 | 14103 | | | |
| | IDENTITY | 45% | 71% | 44% | 52% | | 47% | 100% | 60% | 44% | | | |
| | COVERAGE | 100% | 99% | 100% | 99% | | 99% | 101% | 99% | 100% | | | |
| SAU300156 | SeqID | | | | | | | 13036 | | | | | |
| | IDENTITY | | | | | | | 100% | | | | | |
| | COVERAGE | | | | | | | 100% | | | | | |
| SAU300191 | SeqID | | 10562 | | 11519 | | 11844 | 12367 | 13522 | | | | |
| | IDENTITY | | 43% | | 39% | | 32% | 100% | 41% | | | | |
| | COVERAGE | | 103% | | 91% | | 72% | 101% | 104% | | | | |
| SAU300572 | SeqID | | | | 11522 | | | 12717 | | | | | |
| | IDENTITY | | | | 32% | | | 100% | | | | | |
| | COVERAGE | | | | 108% | | | 100% | | | | | |
| SAU300617 | SeqID | | 10851 | | | | | 12513 | 13289 | | | | |
| | IDENTITY | | 50% | | | | | 100% | 49% | | | | |
| | COVERAGE | | 97% | | | | | 100% | 97% | | | | |
| SAU300713 | SeqID | | 10767 | | | | 11823 | 13058 | | | | | |
| | IDENTITY | | 26% | | | | 30% | 100% | | | | | |
| | COVERAGE | | 83% | | | | 93% | 100% | | | | | |
| SAU300719 | SeqID | 10468 | 10611 | 11246 | 11380 | 11644 | 11887 | 12987 | 13456 | 13726 | | | |
| | IDENTITY | 46% | 34% | 34% | 30% | 30% | 40% | 100% | 33% | 34% | | | |
| | COVERAGE | 100% | 87% | 101% | 94% | 101% | 100% | 101% | 96% | 100% | | | |
| SAU300732 | SeqID | 10282 | 10682 | | | | | 13061 | 13394 | | | | |
| | IDENTITY | 26% | 51% | | | | | 100% | 49% | | | | |
| | COVERAGE | 71% | 88% | | | | | 100% | 86% | | | | |
| SAU300825 | SeqID | | 10655 | | | | | 13068 | 13671 | | | | |
| | IDENTITY | | 52% | | | | | 100% | 41% | | | | |
| | COVERAGE | | 97% | | | | | 100% | 97% | | | | |
| SAU300975 | SeqID | | 10604 | | | | | 12203 | | | | | |
| | IDENTITY | | 30% | | | | | 100% | | | | | |
| | COVERAGE | | 72% | | | | | 102% | | | | | |
| SAU300998 | SeqID | | 10820 | | | | | 13077 | 13489 | | | | |
| | IDENTITY | | 40% | | | | | 100% | 40% | | | | |
| | COVERAGE | | 99% | | | | | 102% | 99% | | | | |
| SAU301004 | SeqID | | 10744 | | | | | 13079 | | | | | |
| | IDENTITY | | 40% | | | | | 100% | | | | | |
| | COVERAGE | | 101% | | | | | 100% | | | | | |
| SAU301030 | SeqID | | | | | | | 13080 | | | | | |
| | IDENTITY | | | | | | | 100% | | | | | |
| | COVERAGE | | | | | | | 100% | | | | | |
| SAU301080 | SeqID | | | | | | | 13083 | | | | | |
| | IDENTITY | | | | | | | 100% | | | | | |
| | COVERAGE | | | | | | | 100% | | | | | |
| SAU301118 | SeqID | 10242 | 10808 | 11092 | | 11653 | | 12904 | | 13795 | | | |
| | IDENTITY | 47% | 58% | 48% | | 53% | | 100% | | 48% | | | |
| | COVERAGE | 98% | 98% | 91% | | 78% | | 100% | | 96% | | | |
| SAU301133 | SeqID | | 10898 | | | | | 13087 | 13443 | | | | |
| | IDENTITY | | 39% | | | | | 100% | 30% | | | | |
| | COVERAGE | | 96% | | | | | | 100% | 93% | | | |
| SAU301223 | SeqID | 10297 | 10640 | 10964 | 11323 | | 11783 | 13090 | 13664 | 13737 | | | |
| | IDENTITY | 31% | 50% | 31% | 32% | | 34% | 100% | 48% | 32% | | | |
| | COVERAGE | 104% | 99% | 102% | 90% | | 102% | 100% | 98% | 104% | | | |
| SAU301230 | SeqID | 10252 | 10877 | 11010 | | 11669 | 11956 | 13092 | 13506 | 13704 | | | |
| | IDENTITY | 52% | 52% | 63% | | 52% | 59% | 100% | 59% | 52% | | | |
| | COVERAGE | 95% | 92% | 74% | | 95% | 77% | 100% | 92% | 95% | | | |
| SAU301268 | SeqID | | | | | | | 13102 | | | | | |
| | IDENTITY | | | | | | | 100% | | | | | |
| | COVERAGE | | | | | | | 100% | | | | | |
| SAU301275 | SeqID | 10048 | 10926 | 11014 | 11511 | | 11934 | 13103 | 13366 | 13897 | | | |
| | IDENTITY | 54% | 47% | 55% | 50% | | 53% | 100% | 46% | 54% | | | |
| | COVERAGE | 99% | 84% | 97% | 97% | | 97% | 101% | 84% | 99% | | | |
| SAU301357 | SeqID | | 10696 | 11063 | | 11766 | | 12859 | 13354 | | | | |
| | IDENTITY | | 74% | 32% | | 33% | | 100% | 76% | | | | |
| | COVERAGE | | 98% | 80% | | 93% | | 101% | 100% | | | | |
| SAU301433 | SeqID | | | | | | | 12845 | 13393 | | | | |
| | IDENTITY | | | | | | | 100% | 26% | | | | |
| | COVERAGE | | | | | | | 100% | 91% | | | | |
| SAU301465 | SeqID | 10210 | 10663 | 11214 | 11554 | | 11921 | 13013 | 13418 | 13925 | | | |
| | IDENTITY | 29% | 54% | 32% | 37% | | 28% | 100% | 52% | 29% | | | |
| | COVERAGE | 100% | 104% | 104% | 100% | | 101% | 100% | 103% | 102% | | | |
| SAU301472 | SeqID | 10157 | | | | | | 12925 | | | | | |
| | IDENTITY | 36% | | | | | | 100% | | | | | |
| | COVERAGE | 85% | | | | | | 100% | | | | | |
| SAU301592 | SeqID | | | | | | | 13137 | | | | | |
| | IDENTITY | | | | | | | 100% | | | | | |
| | COVERAGE | | | | | | | 100% | | | | | |
| SAU301620 | SeqID | | | | | | | 13140 | | | | | |
| | IDENTITY | | | | | | | 100% | | | | | |
| | COVERAGE | | | | | | | 100% | | | | | |
| SAU301758 | SeqID | | | | | | | 13156 | | | | | |
| | IDENTITY | | | | | | | 100% | | | | | |
| | COVERAGE | | | | | | | 100% | | | | | |
| SAU301773 | SeqID | | | | | | | 12729 | | | | | |
| | IDENTITY | | | | | | | 100% | | | | | |
| | COVERAGE | | | | | | | 100% | | | | | |
| SAU301829 | SeqID | 10107 | | | 11309 | | 11857 | 13162 | 13248 | 13935 | | | |
| | IDENTITY | 45% | | | 40% | | 42% | 100% | 38% | 41% | | | |
| | COVERAGE | 98% | | | 97% | | 96% | 100% | 106% | 99% | | | |
| SAU301869 | SeqID | | 10732 | | 11373 | | | 12903 | | | | | |
| | IDENTITY | | 30% | | 36% | | | 100% | | | | | |
| | COVERAGE | | 80% | | 95% | | | 100% | | | | | |
| SAU301898 | SeqID | | 10932 | | | | | 13057 | | | | | |
| | IDENTITY | | 27% | | | | | 100% | | | | | |
| | COVERAGE | | 71% | | | | | 100% | | | | | |
| SAU302060 | SeqID | | | | | | | 13042 | | | | | |
| | IDENTITY | | | | | | | 100% | | | | | |
| | COVERAGE | | | | | | | 100% | | | | | |
| SAU302513 | SeqID | | | | | | | 12851 | | | | | |
| | IDENTITY | | | | | | | 100% | | | | | |
| | COVERAGE | | | | | | | 100% | | | | | |
| SAU302626 | SeqID | | | | | | | 13105 | | | | | |
| | IDENTITY | | | | | | | 100% | | | | | |
| | COVERAGE | | | | | | | 100% | | | | | |
| SAU302685 | SeqID | | | | | | | 13113 | | | | | |
| | IDENTITY | | | | | | | 100% | | | | | |
| | COVERAGE | | | | | | | 100% | | | | | |
| SAU302698 | SeqID | | | | | | | 12725 | | | | | |
| | IDENTITY | | | | | | | 100% | | | | | |
| | COVERAGE | | | | | | | 100% | | | | | |
| SAU302699 | SeqID | | | | | | | 13115 | | | | | |
| | IDENTITY | | | | | | | 100% | | | | | |
| | COVERAGE | | | | | | | 100% | | | | | |
| SAU302805 | SeqID | | | | 11345 | | 13133 | | | | | | |
| | IDENTITY | | | | 33% | | 100% | | | | | | |
| | COVERAGE | | | | 75% | | 101% | | | | | | |
| SAU302901 | SeqID | | | | | | | 12872 | | | | | |
| | IDENTITY | | | | | | | 100% | | | | | |
| | COVERAGE | | | | | | | 100% | | | | | |
| SAU30293 | 1 | SeqID | | | | | | | 13155 | | | | |
| | IDENTITY | | | | | | | 100% | | | | | |
| | COVERAGE | | | | | | | 100% | | | | | |
| SAU302950 | SeqID | | | | | | | 12664 | | | | | |
| | IDENTITY | | | | | | | 100% | | | | | |
| | COVERAGE | | | | | | | 101% | | | | | |
| SAU302956 | SeqID | 10023 | | 11256 | | 11742 | 12044 | 12930 | 13372 | 14018 | | | |
| | IDENTITY | 32% | | 28% | | 31% | 26% | 100% | 31% | 32% | | | |
| | COVERAGE | 88% | | 88% | | 88% | 86% | 101% | 88% | 88% | | | |
| ECO100078 | SeqID | 10023 | | 11256 | | 11742 | 12044 | | 13595 | 14018 | | | |
| | IDENTITY | 100% | | 66% | | 95% | 65% | | 41% | 97% | | | |
| | COVERAGE | 100% | | 98% | | 100% | 99% | | 97% | 100% | | | |
| ECO100252 | SeqID | 10052 | | | 11503 | | 12078 | 12626 | | 13932 | | | |
| | IDENTITY | 100% | | | 41% | | 48% | 38% | | 40% | | | |
| | COVERAGE | 100% | | | 99% | | 96% | 93% | | 93% | | | |
| ECO100397 | SeqID | 10064 | 10781 | 10993 | 11499 | | 11959 | 12884 | 13614 | 13915 | | | |
| | IDENTITY | 100% | 50% | 71% | 38% | | 71% | 45% | 47% | 94% | | | |
| | COVERAGE | 100% | 96% | 100% | 97% | | 97% | 97% | 97% | 99% | | | |
| ECO100398 | SeqID | 10065 | 10653 | 10992 | 11311 | | 11958 | 12883 | 13177 | 13916 | | | |
| | IDENTITY | 100% | 53% | 81% | 46% | | 71% | 57% | 50% | 98% | | | |
| | COVERAGE | 100% | 95% | 101% | 98% | | 99% | 95% | 95% | 100% | | | |
| ECO100990 | SeqID | 10120 | | | | 11768 | | | | | | | |
| | IDENTITY | 100% | | | | 72% | | | | | | | |
| | COVERAGE | 100% | | | | 82% | | | | | | | |
| ECO102108 | SeqID | 10214 | 10608 | 11129 | | 11757 | 11852 | | 13627 | 13931 | | | |
| | IDENTITY | 100% | 36% | 74% | | 94% | 36% | | 36% | 96% | | | |
| | COVERAGE | 100% | 96% | 100% | | 100% | 97% | | 97% | 73% | | | |
| ECO102262 | SeqID | 10228 | | 11204 | | 11631 | 12038 | 13132 | | 13963 | | | |
| | IDENTITY | 100% | | 42% | | 86% | 51% | 35% | | 87% | | | |
| | COVERAGE | 100% | | 100% | | 81% | 100% | 100% | | 100% | | | |
| ECO102447 | SeqID | 10247 | | | | | 11812 | | | 13948 | | | |
| | IDENTITY | 100% | | | | | 47% | | | 99% | | | |
| | COVERAGE | 100% | | | | | 93% | | | 96% | | | |
| ECO102539 | SeqID | 10258 | 10628 | 11134 | 11489 | | 5192 | 12526 | 13636 | 14088 | | | |
| | IDENTITY | 100% | 46% | 77% | 48% | | 71% | 52% | 47% | 97% | | | |
| | COVERAGE | 100% | 101% | 100% | | 100% | 100% | 100% | 82% | 100% | | | |
| ECO102620 | SeqID | 10266 | 10510 | 11269 | 11524 | | 11819 | 12915 | 13279 | 14049 | | | |
| | IDENTITY | 100% | 51% | 26% | 30% | | 28% | 42% | 49% | 89% | | | |
| | COVERAGE | 100% | 93% | 80% | 94% | | 91% | 96% | 101% | 99% | | | |
| ECO103101 | SeqID | 10315 | 10763 | 11215 | 11615 | 11716 | 12052 | | 13662 | 13764 | | | |
| | IDENTITY | 100% | 37% | 73% | 26% | 96% | 64% | | 33% | 94% | | | |
| | COVERAGE | 100% | 74% | 100% | 76% | 100% | 100% | | 74% | 101% | | | |
| ECO104120 | SeqID | 10462 | 10609 | 11034 | | 11726 | 11853 | | | 13887 | | | |
| | IDENTITY | 100% | 29% | 34% | | 87% | 28% | | | 37% | | | |
| | COVERAGE | 100% | 79% | 89% | | 100% | 89% | | | 92% | | | |
| ECO104268 | SeqID | 10475 | 10607 | | | | | 12370 | 13166 | 13707 | | | |
| | IDENTITY | 100% | 43% | | | | | 43% | 38% | 95% | | | |
| | COVERAGE | 100% | 92% | | | | | 99% | 92%100% | | | | |
| KPN100432 | SeqID | 10258 | 10736 | 11134 | 11310 | 11628 | 5192 | 12789 | 13636 | 14088 | | | |
| | IDENTITY | 90% | 37% | 62% | 37% | 100% | 62% | 41% | 47% | 92% | | | |
| | COVERAGE | 100% | 97% | 100% | 93% | 101% 97% | 86% | 87%101% | | | | | |
| KPN100854 | SeqID | 10086 | 10652 | 11197 | 11565 | 11630 | 11862 | | 13389 | 14060 | | | |
| | IDENTITY | 35% | 29% | 26% | 27% | 100% | 42% | | 32% | 35% | | | |
| | COVERAGE | 74% | 72% | 72% | 85% | 100% 77% | | 71% | 74% | | | | |
| KPN101022 | SeqID | 10475 | 10607 | | | 11642 | | 12370 | 13166 | 13707 | | | |
| | IDENTITY | 90% | 29% | | | 100% | | 27% | 26% | 91% | | | |
| | COVERAGE | 100% | 77% | | | 101% | | 101% | 79%101% | | | | |
| KPN101026 | SeqID | 10228 | | 11204 | | 11631 | 12038 | 13132 | | 13963 | | | |
| | IDENTITY | 86% | | 44% | | 100% | 54% | 37% | | 85% | | | |
| | COVERAGE | 99% | | 97% | | 100% 98% | 99% | | 99% | | | | |
| KPN101729 | SeqID | 11045 | 11467 | 11647 | 12067 | 13032 | | | | | | | |
| | IDENTITY | 50% | 50% | 100% | 63% | 63% | | | | | | | |
| | COVERAGE | 96% | 96% | 102% 96% | 96% | | | | | | | | |
| KPN101750 | SeqID | 10052 | | | 11503 | 11652 | 12078 | 12626 | | 13918 | | | |
| | IDENTITY | 94% | | | 38% | 100% | 47% | 37% | | 34% | | | |
| | COVERAGE | 100% | | | 103% | 100% 100% | 96% | | 100% | | | | |
| KPN102057 | SeqID | 10406 | 10892 | 11035 | | 11661 | 11854 | 13153 | | 13883 | | | |
| | IDENTITY | 29% | 30% | 30% | | 100% | 27% | 28% | | 29% | | | |
| | COVERAGE | 96% | 96% | 84% | | 100% | 97% | 85% | | 96% | | | |
| KPN102638 | SeqID | 10266 | 10510 | | 11524 | 11667 | | 12915 | 13557 | 14049 | | | |
| | IDENTITY | 77% | 51% | | 29% | 100% | | 44% | 50% | 77% | | | |
| | COVERAGE | 79% | 79% | | 83% | 100% | | 80% | 79% | 79% | | | |
| KPN103882 | SeqID | 10315 | 10763 | 11215 | 11454 | 11716 | 12052 | | 13662 | 13764 | | | |
| | IDENTITY | 96% | 38% | 73% | 26% | 100% | 65% | | 33% | 93% | | | |
| | COVERAGE | 100% | 74% | 100% | 77% | 100% | 100% | | 74% | 101% | | | |
| KPN104183 | SeqID | 10065 | 10653 | 10992 | 11490 | 11650 | 11958 | 12883 | 13177 | 13916 | | | |
| | IDENTITY | 97% | 56% | 80% | 46% | 100% | 80% | 60% | 55% | 98% | | | |
| | COVERAGE | 85% | 74% | 89% | 86% | 100% | 85% | 74% | 74% | 85% | | | |
| KPN104281 | SeqID | 10023 | | 11256 | | 11742 | 12044 | | 13595 | 14018 | | | |
| | IDENTITY | 95% | | 68% | | 100% | 66% | | 41% | 95% | | | |
| | COVERAGE | 94% | | 92% | | 101% | 94% | | 91% | 101% | | | |
| KPN104538 | SeqID | 10462 | 10609 | 11034 | | 11726 | 11853 | | | 13887 | | | |
| | IDENTITY | 87% | 27% | 35% | | 100% | 29% | | | 38% | | | |
| | COVERAGE | 100% | 87% | 89% | | 100% | 89% | | | 94% | | | |
| KPN104716 | SeqID | 10214 | 10608 | 11129 | | 1175711852 | | 13627 | 13931 | | | | |
| | IDENTITY | 94% | 36% | 75% | | 100% | 36% | | 35% | 94% | | | |
| | COVERAGE | 100% | 96% | 100% | | 100% | 97% | | 97% | 73% | | | |
| KPN105779 | SeqID | | | | | 11770 | 12103 | | | | | | |
| | IDENTITY | | | | | 100% | 28% | | | | | | |
| | COVERAGE | | | | | 101% | 99% | | | | | | |
| KPN106659 | SeqID | 10064 | 10781 | 10993 | | 116491 | 1959 | 12884 | 13614 | 13915 | | | |
| | IDENTITY | 90% | 58% | 72% | | 100% | 74% | 51% | 58% | 91% | | | |
| | COVERAGE | 80% | 70% | 75% | | 101% | 74% | 72% | 70% | 81% | | | |
| KPN106840 | SeqID | 10259 | 10857 | 10978 | | 11664 | 12026 | 12182 | 13691 | 14087 | | | |
| | IDENTITY | 91% | 44% | 74% | | 100% | 55% | 38% | 42% | 91% | | | |
| | COVERAGE | 100% | 101% | 98% | | 100% | 99% | 94% | 92% | 100% | | | |
| KPN107776 | SeqID | 10222 | | 11132 | | 11771 | 11810 | | | 13936 | | | |
| | IDENTITY | 78% | | 37% | | 100% | 35% | | | 80% | | | |
| | COVERAGE | 98% | | 89% | | 102% | 87% | | | 98% | | | |
| SAU100968 | SeqID | 10064 | 10781 | 10993 | 11499 | | 11959 | 12643 | 13614 | 13915 | | | |
| | IDENTITY | 45% | 62% | 44% | 36% | | 46% | 100% | 62% | 46% | | | |
| | COVERAGE | 97% | 97% | 100% | 99% | | 97% | 100% | 98% | 97% | | | |
| SAU201145 | SeqID | 10064 | 10781 | 10993 | 11499 | | 11959 | 12884 | 13614 | 13915 | | | |
| | IDENTITY | 45% | 62% | 44% | 36% | | 46% | 100% | 62% | 46% | | | |
| | COVERAGE | 97% | 97% | 100% | 99% | | 97% | 100% | 98% | 97% | | | |
| SPN101971 | SeqID | 10064 | 10781 | 10993 | 11499 | | 11959 | 12884 | 13287 | 13915 | | | |
| | IDENTITY | 46% | 77% | 42% | 36% | | 48% | 62% | 100% | 46% | | | |
| | COVERAGE | 100% | 99% | 102% | 100% | | 100% | 99% | 100% | 100% | | | |
| SPN201024 | SeqID | 10064 | 10781 | 10993 | 11499 | | 11959 | 12884 | 13614 | 13915 | | | |
| | IDENTITY | 46% | 77% | 43% | 36% | | 49% | 62% | 100% | 46% | | | |
| | COVERAGE | 99% | 99% | 102% | 101% | | 99% | 99% | 100% | 99% | | | |
| STY000277 | SeqID | 10475 | 10607 | | | | | 1237013166 | 13707 | | | | |
| | IDENTITY | 95% | 44% | | | | | 42% | 38% | 100% | | | |
| | COVERAGE | 100% | 91% | | | | | 99% | 96%100% | | | | |
| STY000625 | SeqID | 10421 | | | | | | | | 13784 | | | |
| | IDENTITY | 93% | | | | | | | | 100% | | | |
| | COVERAGE | 100% | | | | | | | | 101% | | | |
| STY000773 | SeqID | 10315 | 10763 | 11215 | 11454 | 11716 | 12052 | | 13662 | 13764 | | | |
| | IDENTITY | 94% | 36% | 71% | 26% | 93% | 62% | | 31% | 100% | | | |
| | COVERAGE | 100% | 74% | 100% | 77% | 100% | 100% | | 74% | 100% | | | |
| STY001430 | SeqID | 10064 | 10781 | 10993 | 11499 | | 11959 | 1288413614 | 13915 | | | | |
| | IDENTITY | 94% | 49% | 70% | 37% | | 70% | 46% | 47% | 100% | | | |
| | COVERAGE | 100% | 96% | 101% | 98% | | 98% | 97% | 98% | 100% | | | |
| STY001433 | SeqID | 10065 | 10653 | 10992 | 11311 | | 11958 | 12883 | 13177 | 13916 | | | |
| | IDENTITY | 98% | 53% | 82% | 46% | | 72% | 58% | 50% | 100% | | | |
| | COVERAGE | 99% | 94% | 100% | 97% | | 99% | 94% | 94% | 100% | | | |
| STY001867 | SeqID | 10247 | | | | | 11812 | | | 13948 | | | |
| | IDENTITY | 99% | | | | | 47% | | | 100% | | | |
| | COVERAGE | 98% | | | | | 96% | | | 100% | | | |
| STY002995 | SeqID | 10023 | | 11256 | | 11742 | 12044 | | 13595 | 14018 | | | |
| | IDENTITY | 97% | | 67% | | 95% | 65% | | 40% | 100% | | | |
| | COVERAGE | 94% | | 92% | | 101% | 94% | | 91% | 101% | | | |
| STY003357 | SeqID | 10228 | | 11204 | | 11631 | 12038 | 13132 | | 13963 | | | |
| | IDENTITY | 87% | | 42% | | 85% | 49% | 36% | | 100% | | | |
| | COVERAGE | 100% | | 100% | | 81% | 101% | 100% | | 100% | | | |
| PA0028 | SeqID | | | | | | 5053 | | | | | | |
| | COVERAGE | | | | | | 100% | | | | | | |
| | IDENTITY | | | | | | 100% | | | | | | |
| PA0120 | SeqID | 10386 | | 10959 | | | 5054 | | | 13899 | | | |
| | COVERAGE | 96% | | 94% | | | 100% | | | 95% | | | |
| | IDENTITY | 28% | | 28% | | | 100% | | | 28% | | | |
| PA0129 | SeqID | 10265 | | | 11388 | | 5055 | 12844 | | 14048 | | | |
| | COVERAGE | 93% | | | 91% | | 100% | 94% | | 91% | | | |
| | IDENTITY | 67% | | | 32% | | 100% | 36% | | 67% | | | |
| PA0141 | SeqID | | | | | | 5056 | | | | | | |
| | COVERAGE | | | | | | 100% | | | | | | |
| | IDENTITY | | | | | | 100% | | | | | | |
| PA0221 | SeqID | | | 11250 | 11386 | 11701 | 5057 | 12781 | | 13778 | | | |
| | COVERAGE | | | 73% | 77% | 83% | 100% | 96% | | 77% | | | |
| | IDENTITY | | | 32% | 26% | 28% | 100% | 28% | | 29% | | | |
| PA0265 | SeqID | 10264 | 10550 | | 11466 | | 5058 | 12375 | 13316 | 14047 | | | |
| | COVERAGE | 100% | 97% | | 99% | | 100% | 96% | 91% | 100% | | | |
| | IDENTITY | 81% | 35% | | 26% | | 100% | 38% | 34% | 80% | | | |
| PA0321 | SeqID | | | | | | 5059 | | | | | | |
| | COVERAGE | | | | | | 100% | | | | | | |
| | IDENTITY | | | | | | 100% | | | | | | |
| PA0337 | SeqID | 10278 | 10785 | 11275 | | | 5060 | 12351 | 13281 | 13880 | | | |
| | COVERAGE | 99% | 73% | 72% | | | 100% | 72% | 73% | 99% | | | |
| | IDENTITY | 43% | 35% | 37% | | | 100% | 36% | 35% | 42% | | | |
| PA0353 | SeqID | 10408 | 11088 | | 11397 | 11749 | 5061 | 12159 | 13511 | 14034 | | | |
| | COVERAGE | 97% | | 100% | 88% | 101% | 100% | 100% | 96% | 101% | | | |
| | IDENTITY | 74% | | 75% | 28% | 74%100% | 45% | 38% | 74% | | | | |
| PA0378 | SeqID | 10324 | | 11130 | | | 5062 | | | 13730 | | | |
| | COVERAGE | 94% | | 80% | | | 100% | | | 95% | | | |
| | IDENTITY | 52% | | 49% | | | 100% | | | 53% | | | |
| PA0401 | SeqID | 10078 | 10858 | | | | 5063 | 12993 | 13560 | 13723 | | | |
| | COVERAGE | 99% | 100% | | | | 100% | 96% | 100% | 99% | | | |
| | IDENTITY | 26% | 31% | | | | 100% | 33% | 33% | 26% | | | |
| PA0413 | SeqID | | | | | | 5064 | | | | | | |
| | COVERAGE | | | | | | 100% | | | | | | |
| | IDENTITY | | | | | | 100% | | | | | | |
| PA0414 | SeqID | | | | | | 5065 | | | | | | |
| | COVERAGE | | | | | | 100% | | | | | | |
| | IDENTITY | | | | | | 100% | | | | | | |
| PA0419 | SeqID | 10296 | 10871 | 11003 | | 11660 | 5066 | 12971 | 13461 | 13738 | | | |
| | COVERAGE | 100% | 93% | 102% | | 78% | 100% | 100% | 91% | 100% | | | |
| | IDENTITY | 46% | 29% | 45% | | 47% | 100% | 27% | 29% | 47% | | | |
| PA0423 | SeqID | 10123 | | | 11424 | | 5067 | 12708 | | 14038 | | | |
| | COVERAGE | 99% | | | 97% | | 100% | 75% | | 99% | | | |
| | IDENTITY | 75% | | | 32% | | 100% | 32% | | 76% | | | |
| PA0469 | SeqID | | | | | | 5068 | | | | | | |
| | COVERAGE | | | | | | 100% | | | | | | |
| | IDENTITY | | | | | | 100% | | | | | | |
| PA0472 | SeqID | 10471 | | | | | 5069 | | | | | | |
| | COVERAGE | 88% | | | | | 100% | | | | | | |
| | IDENTITY | 47% | | | | | 100% | | | | | | |
| PA0506 | SeqID | | | | | | 5070 | | | | | | |
| | COVERAGE | | | | | | 100% | | | | | | |
| | IDENTITY | | | | | | 100% | | | | | | |
| PA0600 | SeqID | | | | | | 5071 | | | | | | |
| | COVERAGE | | | | | | 100% | | | | | | |
| | IDENTITY | | | | | | 100% | | | | | | |
| PA0642 | SeqID | | | | | | 5072 | | | | | | |
| | COVERAGE | | | | | | 100% | | | | | | |
| | IDENTITY | | | | | | 100% | | | | | | |
| PA0650 | SeqID | 10150 | | 11237 | 11581 | | 5073 | 12153 | 13459 | 13846 | | | |
| | COVERAGE | 95% | | 83% | 93% | | 100% | 76% | 95% | 95% | | | |
| | IDENTITY | 38% | | 38% | 35% | | 100% | 34% | 38% | 39% | | | |
| PA0715 | SeqID | | | | | | 5074 | | | | | | |
| | COVERAGE | | | | | | 100% | | | | | | |
| | IDENTITY | | | | | | 100% | | | | | | |
| PA0788 | SeqID | | | | | | 5075 | | | | | | |
| | COVERAGE | | | | | | 100% | | | | | | |
| | IDENTITY | | | | | | | 100% | | | | | |
| PA0882 | SeqID | 10233 | | | | | 5076 | | | 14013 | | | |
| | COVERAGE | 85% | | | | | 100% | | | 101% | | | |
| | IDENTITY | 33% | | | | | 100% | | | 28% | | | |
| PA0934 | SeqID | 10276 | 10876 | 11006 | | 11753 | 5077 | 12646 | 13483 | | | | |
| | COVERAGE | 101% | 93% | 101% | | 80% | 100% | 92% | 94% | | | | |
| | IDENTITY | 47% | 40% | 46% | | 37% | 100% | 39% | 38% | | | | |
| PA0938 | SeqID | | | | | | 5078 | | | | | | |
| | COVERAGE | | | | | | 100% | | | | | | |
| | IDENTITY | | | | | | | 100% | | | | | |
| PA1019 | SeqID | 10467 | 10592 | 11180 | | | 5079 | | | | | | |
| | COVERAGE | 88% | 84% | 88% | | | 100% | | | | | | |
| | IDENTITY | 26% | 25% | 28% | | | 100% | | | | | | |
| PA1072 | SeqID | 10377 | | | | | 5080 | | 13410 | 13813 | | | |
| | COVERAGE | 100% | | | | | 100% | | 71% | 100% | | | |
| | IDENTITY | 62% | | | | | 100% | | 36% | 61% | | | |
| PA1115 | SeqID | | | | | | 5081 | | | | | | |
| | COVERAGE | | | | | | 100% | | | | | | |
| | IDENTITY | | | | | | 100% | | | | | | |
| PA1270 | SeqID | 10328 | | | | 11751 | 5082 | | | 13946 | | | |
| | COVERAGE | 76% | | | | 79% | 100% | | | 76% | | | |
| | IDENTITY | 26% | | | | 25% | 100% | | | 26% | | | |
| PA1301 | SeqID | 10470 | | | | | 5083 | | | | | | |
| | COVERAGE | 96% | | | | | 100% | | | | | | |
| | IDENTITY | 28% | | | | | 100% | | | | | | |
| PA1360 | SeqID | 10104 | | | | | 5084 | | 13282 | 14000 | | | |
| | COVERAGE | 92% | | | | | 100% | | 97% | 92% | | | |
| | IDENTITY | 63% | | | | | 100% | | 25% | 63% | | | |
| PA1365 | SeqID | | | | | | 5085 | | | | | | |
| | COVERAGE | | | | | | 100% | | | | | | |
| | IDENTITY | | | | | | 100% | | | | | | |
| PA1398 | SeqID | | | | | | 5086 | | | | | | |
| | COVERAGE | | | | | | 100% | | | | | | |
| | IDENTITY | | | | | | 100% | | | | | | |
| PA1462 | SeqID | | 10915 | | 11559 | | 5087 | | | | | | |
| | COVERAGE | | 98% | | 101% | | 100% | | | | | | |
| | IDENTITY | | | 29% | | 30% | | 100% | | | | | |
| PA1493 | SeqID | 110423 | | | | 11718 | 5088 | | | 13786 | | | |
| | COVERAGE | 92% | | | | 97% | 100% | | | 92% | | | |
| | IDENTITY | 56% | | | | 49% | 100% | | | 56% | | | |
| PA1547SeqID | | | | 11377 | | 5089 | | | | | | | |
| | COVERAGE | | | | 88% | | 100% | | | | | | |
| | IDENTITY | | | | 28% | | 100% | | | | | | |
| SeqID | 110091 | | | | | 5090 | 12990 | | 13890 | | | | |
| | COVERAGE | 101% | | | | | 100% | 96% | | 81% | | | |
| | IDENTITY | 37% | | | | | 100% | 26% | | 32% | | | |
| PA1684 | SeqID | | | | | 11693 | 5091 | | | | | | |
| | COVERAGE | | | | | 99% | 100% | | | | | | |
| | IDENTITY | | | | | 59% | 100% | | | | | | |
| PA1868 | SeqID | 10361 | | | | | 5092 | | | | | | |
| | COVERAGE | 82% | | | | | 100% | | | | | | |
| | IDENTITY | 35% | | | | | 100% | | | | | | |
| PA1876 | SeqID | | | | | 11746 | 5093 | | 14036 | | | | |
| | COVERAGE | | | | | 76% | 100% | | 93% | | | | |
| | IDENTITY | | | | | 40% | 100% | | 39% | | | | |
| PA1918 | SeqID | 10153 | | 11033 | | | 5094 | | | 13745 | | | |
| | COVERAGE | 79% | | 82% | | | 100% | | | 79% | | | |
| | IDENTITY | 31% | | 28% | | | 100% | | | 28% | | | |
| PA1986 | SeqID | | | | | | 5095 | | | | | | |
| | COVERAGE | | | | | | 100% | | | | | | |
| | IDENTITY | | | | | | 100% | | | | | | |
| PA2009 | SeqID | | | | | | 5096 | | | | | | |
| | COVERAGE | | | | | | 100% | | | | | | |
| | IDENTITY | | | | | | 100% | | | | | | |
| PA2083 | SeqID | 10253 | | | | 11692 | 5097 | | | | | | |
| | COVERAGE | 87% | | | | 85% | 100% | | | | | | |
| | IDENTITY | 31% | | | | 35% | 100% | | | | | | |
| PA2101 | SeqID | 10198 | | | | | 5098 | | 13282 | 13861 | | | |
| | COVERAGE | 92% | | | | | 100% | | 88% | 95% | | | |
| | IDENTITY | 30% | | | | | 100% | | 25% | 28% | | | |
| PA2108 | SeqID | 10109 | | 11257 | | | 5099 | 12943 | 13625 | 13996 | | | |
| | COVERAGE | 96% | | 95% | | | 100% | 94% | 90% | 96% | | | |
| | IDENTITY | 37% | | 27% | | | 100% | 34% | 29% | 37% | | | |
| PA2128 | SeqID | 10472 | 10865 | | | 11752 | 5100 | | 13683 | 13893 | | | |
| | COVERAGE | 97% | 96% | | | 86% | 100% | | 80% | 97% | | | |
| | IDENTITY | 27% | 26% | | | 25% | 100% | | 27% | 33% | | | |
| PA2147 | SeqID | 10181 | | | | | 5101 | | | 13985 | | | |
| | COVERAGE | 98% | | | | | 100% | | | 98% | | | |
| | IDENTITY | 60% | | | | | 100% | | | 59% | | | |
| PA2196 | SeqID | 10169 | | | | | 5102 | | 13852 | | | | |
| | COVERAGE | 99% | | | | | 100% | | 99% | | | | |
| | IDENTITY | 43% | | | | | 100% | | 43% | | | | |
| PA2197 | SeqID | 10160 | | | | | 5103 | 12917 | | 13830 | | | |
| | COVERAGE | 100% | | | | | 100% | 97% | | 100% | | | |
| | IDENTITY | 74% | | | | | 100% | 44% | | 73% | | | |
| PA2222 | SeqID | | | | | | 5104 | | | | | | |
| | COVERAGE | | | | | | 100% | | | | | | |
| | IDENTITY | | | | | | 100% | | | | | | |
| PA2313 | SeqID | | | | | | 5105 | | | | | | |
| | COVERAGE | | | | | | 100% | | | | | | |
| | IDENTITY | | | | | | 100% | | | | | | |
| PA2398 | SeqID | 10132 | | | | | 5106 | | | | | | |
| | COVERAGE | 86% | | | | | 100% | | | | | | |
| | IDENTITY | 35% | | | | | 100% | | | | | | |
| PA2424 | SeqID | | | | | | 5107 | | | | | | |
| | COVERAGE | | | | | | 100% | | | | | | |
| | IDENTITY | | | | | | 100% | | | | | | |
| PA2461 | SeqID | | | | | | 5108 | | | | | | |
| | COVERAGE | | | | | | 100% | | | | | | |
| | IDENTITY | | | | | | 100% | | | | | | |
| PA2470 | SeqID | | | | | | 5109 | 13930 | | | | | |
| | COVERAGE | | | | | | 100% | 98% | | | | | |
| | IDENTITY | | | | | | 100% | 60% | | | | | |
| PA2488 | SeqID | 10189 | | 11172 | | | 5110 | | | 13980 | | | |
| | COVERAGE | 89% | | 70% | | | 100% | | | 87% | | | |
| | IDENTITY | 32% | | 28% | | | 100% | | | 29% | | | |
| PA2494 | SeqID | 10331 | | 11145 | 11516 | | 5111 | | | 13719 | | | |
| | COVERAGE | 99% | | 98% | 100% | | 100% | | | 98% | | | |
| | IDENTITY | 42% | | 31% | 26% | | 100% | | | 41% | | | |
| PA2584 | SeqID | 10195 | 10899 | 10967 | 11504 | | 5112 | 12330 | 13442 | 14058 | | | |
| | COVERAGE | 94% | 99% | 94% | 97% | | 100% | 99% | 92% | 94% | | | |
| | IDENTITY | 60% | 37% | 57% | 38% | | 100% | 41% | 42% | 58% | | | |
| PA2594 | SeqID | 10116 | | | | 11714 | 5113 | | | | | | |
| | COVERAGE | 97% | | | | 80% | 100% | | | | | | |
| | IDENTITY | 41% | | | | 45% | 100% | | | | | | |
| PA2634 | SeqID | 10441 | | | | | 5114 | | | | | | |
| | COVERAGE | 74% | | | | | 100% | | | | | | |
| | IDENTITY | 28% | | | | | 100% | | | | | | |
| PA2641 | SeqID | 10226 | 10566 | | | | 5115 | | | 13959 | | | |
| | COVERAGE | 95% | 89% | | | | 100% | | | 95% | | | |
| | IDENTITY | 80% | 37% | | | | 100% | | | 80% | | | |
| PA2671 | SeqID | | | | | | 5116 | | | | | | |
| | COVERAGE | | | | | | 100% | | | | | | |
| | IDENTITY | | | | | | 100% | | | | | | |
| PA2680 | SeqID | 10444 | 10703 | | | 11730 | 5117 | | | 14029 | | | |
| | COVERAGE | 101% | 74% | | | 90% | 100% | | | 101% | | | |
| | IDENTITY | 42% | 30% | | | 43% | 100% | | | 42% | | | |
| PA2684 | SeqID | 10384 | | | | | 5118 | | | | | | |
| | COVERAGE | 99% | | | | | 100% | | | | | | |
| | IDENTITY | 33% | | | | | 100% | | | | | | |
| PA2726 | SeqID | | | | | | 5119 | | | | | | |
| | COVERAGE | | | | | | 100% | | | | | | |
| | IDENTITY | | | | | | | 100% | | | | | |
| PA2742 | SeqID | 10177 | 10660 | 11222 | 11296 | | 5120 | 12628 | 13302 | | | | |
| | COVERAGE | 91% | 97% | 84% | 89% | | 100% | 97% | 97% | | | | |
| | IDENTITY | 64% | 50% | 67% | 47% | | 100% | 55% | 45% | | | | |
| PA3006 | SeqID | | | | | | 5121 | | | | | | |
| | COVERAGE | | | | | | 100% | | | | | | |
| | IDENTITY | | | | | | 100% | | | | | | |
| PA3011 | SeqID | 10151 | 10695 | 11233 | 11293 | | 5122 | 12339 | | 13848 | | | |
| | COVERAGE | 100% | 79% | 100% | 86% | | 100% | 75% | | 100% | | | |
| | IDENTITY | 68% | 40% | 64% | 39% | | 100% | 42% | | 66% | | | |
| PA3013 | SeqID | 10416 | 10494 | 11095 | 11525 | | 5123 | 12461 | | 13750 | | | |
| | COVERAGE | 98% | 80% | 102% | 102% | | 100% | 102% | | 98% | | | |
| | IDENTITY | 64% | 39% | 43% | 41% | | 100% | 40% | | 64% | | | |
| PA3041 | SeqID | 10307 | | | | | 5124 | | | 13777 | | | |
| | COVERAGE | 88% | | | | | 100% | | | 88% | | | |
| | IDENTITY | 32% | | | | | 100% | | | 32% | | | |
| PA3048 | SeqID | 10117 | | 10966 | | | 5125 | | | 14005 | | | |
| | COVERAGE | 99% | | 75% | | | 100% | | | 99% | | | |
| | IDENTITY | 47% | | 45% | | | 100% | | | 47% | | | |
| PA3068 | SeqID | | | | | | 5126 | | | | | | |
| | COVERAGE | | | | | | 100% | | | | | | |
| | IDENTITY | | | | | | 100% | | | | | | |
| PA3121 | SeqID | 10021 | | 11164 | 11363 | | 5127 | 12156 | | 14017 | | | |
| | COVERAGE | 99% | | 99% | 81% | | 100% | 99% | | 99% | | | |
| | IDENTITY | 63% | | 59% | 26% | | 100% | 56% | | 62% | | | |
| PA3153 | SeqID | | | | | | 5128 | | | | | | |
| | COVERAGE | | | | | | 100% | | | | | | |
| | IDENTITY | | | | | | 100% | | | | | | |
| PA3154 | SeqID | | | | | | 5129 | | | | | | |
| | COVERAGE | | | | | | 100% | | | | | | |
| | IDENTITY | | | | | | 100% | | | | | | |
| PA3160 | SeqID | | | | | | 5130 | | | | | | |
| | COVERAGE | | | | | | 100% | | | | | | |
| | IDENTITY | | | | | | 100% | | | | | | |
| PA3279 | SeqID | | | | | | 5131 | | | | | | |
| | COVERAGE | | | | | | 100% | | | | | | |
| | IDENTITY | | | | | | 100% | | | | | | |
| PA3280 | SeqID | | | | | | 5132 | | | | | | |
| | COVERAGE | | | | | | 100% | | | | | | |
| | IDENTITY | | | | | | 100% | | | | | | |
| PA3374 | SeqID | 10452 | | | | | 5133 | | | | | | |
| | COVERAGE | 99% | | | | | 100% | | | | | | |
| | IDENTITY | 55% | | | | | | 100% | | | | | |
| PA3479 | SeqID | | | | | | 5134 | | | | | | |
| | COVERAGE | | | | | | 100% | | | | | | |
| | IDENTITY | | | | | | 100% | | | | | | |
| PA3484 | SeqID | | | | | | 5135 | | | | | | |
| | COVERAGE | | | | | | 100% | | | | | | |
| | IDENTITY | | | | | | 100% | | | | | | |
| PA3522 | SeqID | 10331 | | 11145 | 11516 | | 5136 | | | 13719 | | | |
| | COVERAGE | 98% | | 99% | 99% | | 100% | | | 99% | | | |
| | IDENTITY | 41% | | 30% | 26% | | 100% | | | 40% | | | |
| PA3643 | SeqID | 10046 | | 11173 | 11378 | | 5137 | | | 13912 | | | |
| | COVERAGE | 99% | | 100% | 79% | | 100% | | | 99% | | | |
| | IDENTITY | 53% | | 51% | 30% | | 100% | | | 52% | | | |
| PA3703 | SeqID | 10194 | | | | | 5138 | | | 13751 | | | |
| | COVERAGE | 100% | | | | | 100% | | | 100% | | | |
| | IDENTITY | 30% | | | | | 100% | | | 31% | | | |
| PA3709 | SeqID | | | | | | 5139 | | | | | | |
| | COVERAGE | | | | | | 100% | | | | | | |
| | IDENTITY | | | | | | 100% | | | | | | |
| PA3716 | SeqID | | | | | | 5140 | | | | | | |
| | COVERAGE | | | | | | 100% | | | | | | |
| | IDENTITY | | | | | | 100% | | | | | | |
| PA3764 | SeqID | 10255 | | 10991 | | | 5141 | | | 13793 | | | |
| | COVERAGE | 94% | | 91% | | | 100% | | | 82% | | | |
| | IDENTITY | 38% | | 41% | | | 100% | | | 39% | | | |
| PA3845 | SeqID | 10277 | | 11200 | | | 5142 | | | 13882 | | | |
| | COVERAGE | 98% | | 98% | | | 100% | | | 98% | | | |
| | IDENTITY | 34% | | 30% | | | 100% | | | 35% | | | |
| PA3866 | SeqID | | | | | | 5143 | | | | | | |
| | COVERAGE | | | | | | 100% | | | | | | |
| | IDENTITY | | | | | | 100% | | | | | | |
| PA3876 | SeqID | 10144 | | | | | 5144 | | | 13840 | | | |
| | COVERAGE | 97% | | | | | 100% | | | 97% | | | |
| | IDENTITY | 61% | | | | | 100% | | | 58% | | | |
| PA3877 | SeqID | 10161 | | | | | 5145 | 12699 | | 13831 | | | |
| | COVERAGE | 98% | | | | | 100% | 92% | | 98% | | | |
| | IDENTITY | 28% | | | | | 100% | 27% | | 27% | | | |
| PA3931 | SeqID | 10050 | 10833 | 11067 | 11460 | 11656 | 5146 | 12548 | 13173 | 13720 | | | |
| | COVERAGE | 82% | 92% | 103% | 92% | 82% | 100% | 96% | 109% | 95% | | | |
| | IDENTITY | 50% | 43% | 41% | 49% | 48% | 100% | 44% | 36% | 35% | | | |
| PA3984 | SeqID | 10087 | | 11002 | | 11674 | 5147 | | | 14061 | | | |
| | COVERAGE | 97% | | 98% | | 91% | 100% | | | 99% | | | |
| | IDENTITY | 40% | | 37% | | 39% | 100% | | | 40% | | | |
| PA4024 | SeqID | 10244 | 10700 | | | 11736 | 5148 | | | 13951 | | | |
| | COVERAGE | 95% | 95% | | | 71% | 100% | | | 95% | | | |
| | IDENTITY | 50% | 50% | | | 72% | 100% | | | 50% | | | |
| PA4027 | SeqID | | | | | | 5149 | | | | | | |
| | COVERAGE | | | | | | 100% | | | | | | |
| | IDENTITY | | | | | | 100% | | | | | | |
| PA4037 | SeqID | 10102 | 10563 | 11194 | 11527 | 11725 | 5150 | 12958 | 13296 | 14002 | | | |
| | COVERAGE | 72% | 83% | 72% | 72% | 72% | 100% | 70% | 71% | 72% | | | |
| | IDENTITY | 35% | 30% | 33% | 34% | 33% | 100% | 35% | 31% | 34% | | | |
| PA4067 | SeqID | 10149 | | | | | 5151 | | | 13845 | | | |
| | COVERAGE | 98% | | | | | 100% | | | 99% | | | |
| | IDENTITY | 44% | | | | | 100% | | | 43% | | | |
| PA4070 | SeqID | 10159 | | | | | 5152 | | | | | | |
| | COVERAGE | 96% | | | | | 100% | | | | | | |
| | IDENTITY | 28% | | | | | 100% | | | | | | |
| PA408 | 1 | SeqID | | | | | | 5153 | | | | | |
| | COVERAGE | | | | | | 100% | | | | | | |
| | IDENTITY | | | | | | 100% | | | | | | |
| PA4105 | SeqID | | | | | | 5154 | | | | | | |
| | COVERAGE | | | | | | 100% | | | | | | |
| | IDENTITY | | | | | | 100% | | | | | | |
| PA4124 | SeqID | | | | | | 5155 | | | 14023 | | | |
| | COVERAGE | | | | | | 100% | | | 93% | | | |
| | IDENTITY | | | | | | 100% | | | 64% | | | |
| PA4125 | SeqID | | | | | | 5156 | | | 14024 | | | |
| | COVERAGE | | | | | | 100% | | | 94% | | | |
| | IDENTITY | | | | | | 100% | | | 67% | | | |
| PA4158 | SeqID | 10080 | 10610 | 11009 | 11379 | 11769 | 5157 | 12297 | | 13725 | | | |
| | COVERAGE | 98% | 95% | 88% | 83% | 74% | 100% | 96% | | 97% | | | |
| | IDENTITY | 61% | 38% | 31% | 28% | 61% | 100% | 50% | | 61% | | | |
| PA4237 | SeqID | 10333 | 10542 | 11123 | 11582 | | 5158 | 12232 | 13224 | 14093 | | | |
| | COVERAGE | 91% | 97% | 98% | 90% | | 100% | 92% | 97% | 91% | | | |
| | IDENTITY | 79% | 43% | 76% | 43% | | 100% | 45% | 42% | 79% | | | |
| PA4242 | SeqID | 10338 | 10538 | 11117 | 11428 | | 5159 | | | | | | |
| | COVERAGE | 100% | 100% | 100% | | 100% | 100% | | | | | | |
| | IDENTITY | 87% | 68% | 76% | 74% | | 100% | | | | | | |
| PA4244 | SeqID | 10340 | 10534 | 11116 | | | 5160 | 12225 | 13217 | 14099 | | | |
| | COVERAGE | 100% | 100% | 100% | 100% | | | 100% | 100% | 100% | | | |
| | IDENTITY | 65% | 46% | 63% | | | 100% | 42% | 43% | 65% | | | |
| PA4245 | SeqID | 10341 | 10532 | 11115 | | | 5161 | 12223 | 13216 | 13812 | | | |
| | COVERAGE | 95% | 98% | 95% | | | 100% | 98% | 98% | 78% | | | |
| | IDENTITY | 56% | 42% | 58% | | | 100% | 42% | 40% | 33% | | | |
| PA4246 | SeqID | 10342 | 10531 | 11114 | 11432 | | 5162 | 12222 | 13215 | 14101 | | | |
| | COVERAGE | 100% | 92% | 99% | 88% | | 100% | 99% | 92% | 100% | | | |
| | IDENTITY | 77% | 52% | 74% | 49% | | 100% | 52% | 53% | 77% | | | |
| PA4247 | SeqID | 10343 | 10530 | 11113 | 11433 | | 5163 | 12221 | 13214 | 14102 | | | |
| | COVERAGE | 99% | 98% | 99% | 97% | | 100% | 98% | 98% | 99% | | | |
| | IDENTITY | 59% | 52% | 63% | 37% | | 100% | 48% | 54% | 59% | | | |
| PA4248 | SeqID | 10344 | 10529 | 11112 | 11434 | | 5164 | 12220 | 13571 | 14103 | | | |
| | COVERAGE | 100% | 99% | 100% | 99% | | 100% | 99% | 99% | 100% | | | |
| | IDENTITY | 62% | 49% | 66% | 50% | | 100% | 43% | 47% | 62% | | | |
| PA4249 | SeqID | 10345 | 10528 | 11111 | 11435 | | 5165 | 13033 | 13212 | 14104 | | | |
| | COVERAGE | 99% | 102% | 99% | 100% | | 100% | 102% | 102% | 99% | | | |
| | IDENTITY | 64% | 46% | 64% | 40% | | 100% | 44% | 47% | 64% | | | |
| PA4250 | SeqID | 10346 | 10599 | 11110 | | | 5166 | 12737 | 13211 | 14105 | | | |
| | COVERAGE | 100% | 100% | 100% | | | 100% | 100% | 100% | 100% | | | |
| | IDENTITY | 69% | 43% | 63% | | | 100% | 46% | 53% | 67% | | | |
| PA4251 | SeqID | 10347 | 10527 | 11109 | 11589 | 11654 | 5167 | 12218 | 13210 | 14106 | | | |
| | COVERAGE | 99% | 99% | 99% | 99% | 99% | 100% | 90% | 98% | 99% | | | |
| | IDENTITY | 69% | 58% | 68% | 48% | 69% | 100% | 63% | 61% | 68% | | | |
| PA4252 | SeqID | 10348 | 10526 | 11108 | | | 5168 | 12217 | 13209 | 14107 | | | |
| | COVERAGE | 97% | 92% | 94% | | | 100% | 98% | 92% | 96% | | | |
| | IDENTITY | 65% | 49% | 62% | | | 100% | 49% | 46% | 64% | | | |
| PA4253 | SeqID | 10349 | 10525 | 11107 | 11436 | | 5169 | 12216 | 13208 | 14108 | | | |
| | COVERAGE | 101% | 100% | 101% | 100% | | 100% | 100% | 100% | 101% | | | |
| | IDENTITY | 85% | 66% | 85% | 65% | | 100% | 66% | 66% | 84% | | | |
| PA4254 | SeqID | 10350 | 10524 | 11106 | 11437 | | 5170 | 12215 | 13207 | | | | |
| | COVERAGE | 90% | 98% | 90% | 84% | | 100% | 89% | 89% | | | | |
| | IDENTITY | 71% | 53% | 62% | 45% | | 100% | 55% | 56% | | | | |
| PA4256 | SeqID | 10352 | 10560 | 11104 | 11439 | | 5171 | 12260 | 13204 | 13968 | | | |
| | COVERAGE | 100% | 100% | 100% | 96% | | 100% | 98% | 98% | 100% | | | |
| | IDENTITY | 77% | 54% | 77% | 65% | | 100% | 58% | 57% | 77% | | | |
| PA4257 | SeqID | 10353 | 10559 | 11103 | 11592 | | 5172 | 12259 | 13203 | 13969 | | | |
| | COVERAGE | 99% | 91% | 100% | 99% | | 100% | 91% | 93% | 99% | | | |
| | IDENTITY | 74% | 61% | 72% | 55% | | 100% | 57% | 59% | 74% | | | |
| PA4258 | SeqID | 10354 | 10558 | 11102 | 11593 | | 5173 | 12258 | 13202 | 13970 | | | |
| | COVERAGE | 100% | 91% | 100% | 95% | | 100% | 99% | 91% | 100% | | | |
| | IDENTITY | 69% | 57% | 70% | 41% | | 100% | 48% | 58% | 69% | | | |
| PA4259 | SeqID | 10355 | 10557 | 11101 | 11594 | | 5174 | 12255 | 13201 | | | | |
| | COVERAGE | 100% | 101% | 100% | 99% | | 100% | 100% | 100% | | | | |
| | IDENTITY | 82% | 70% | 84% | 61% | | 100% | 63% | 67% | | | | |
| PA4262 | SeqID | 10358 | 10549 | 11098 | 11595 | | 5175 | 12240 | 13198 | 13973 | | | |
| | COVERAGE | 100% | 95% | 100% | 96% | | 100% | 101% | 97% | 100% | | | |
| | IDENTITY | 68% | 45% | 72% | 36% | | 100% | 46% | 44% | 68% | | | |
| PA4263 | SeqID | 10359 | | 11097 | 11442 | | 5176 | 12235 | 13197 | 13974 | | | |
| | COVERAGE | 99% | | 98% | 91% | | 100% | 103% | 99% | 99% | | | |
| | IDENTITY | 75% | | 73% | 35% | | 100% | 46% | 51% | 75% | | | |
| PA4264 | SeqID | 10360 | 10533 | 11096 | 11443 | 11643 | 5177 | | 13196 | 13975 | | | |
| | COVERAGE | 100% | 75% | 100% | 95% | 100% | 100% | | 99% | 100% | | | |
| | IDENTITY | 90% | 58% | 92% | 57% | 92% | 100% | | 61% | 91% | | | |
| PA4268 | SeqID | 10365 | 10479 | 11062 | 11409 | | 5178 | 12445 | 13231 | 13967 | | | |
| | COVERAGE | 100% | 111% | 100% | 100% | | 100% | 111% | 111% | 100% | | | |
| | IDENTITY | 89% | 70% | 89% | 75% | | 100% | 68% | 70% | 89% | | | |
| PA4269 | SeqID | 10439 | 10627 | 11036 | 11410 | | 5179 | 12446 | 13646 | 14042 | | | |
| | COVERAGE | 100% | 100% | 100% | 109% | | 100% | 101% | 99% | 100% | | | |
| | IDENTITY | 76% | 46% | 73% | 47% | | 100% | 46% | 45% | 75% | | | |
| PA4271 | SeqID | 10437 | 10615 | 11072 | 11572 | | 5180 | 12449 | 13247 | 14044 | | | |
| | COVERAGE | 100% | 101% | 101% | 102% | | 100% | 98% | 100% | 100% | | | |
| | IDENTITY | 66% | 65% | 66% | 54% | | 100% | 58% | 58% | 64% | | | |
| PA4272 | SeqID | 10436 | 10614 | 11071 | | | 5181 | 12450 | 13246 | 14045 | | | |
| | COVERAGE | 99% | 95% | 100% | | | 100% | 99% | 95% | 99% | | | |
| | IDENTITY | 68% | 40% | 66% | | | 100% | 39% | 42% | 65% | | | |
| PA4316 | SeqID | 10200 | | 11235 | | | 5182 | | | 13821 | | | |
| | COVERAGE | 88% | | 90% | | | 100% | | | 91% | | | |
| | IDENTITY | 51% | | 47% | | | 100% | | | 51% | | | |
| PA4332 | SeqID | | | | | | 5183 | | | | | | |
| | COVERAGE | | | | | | 100% | | | | | | |
| | IDENTITY | 1 | | | | | | 100% | | | | | |
| PA4347 | SeqID | | | | | 11699 | 5184 | | | | | | |
| | COVERAGE | | | | | 86% | 100% | | | | | | |
| | IDENTITY | | | | | 27% | 100% | | | | | | |
| PA4363 | SeqID | 10292 | | | | 11740 | 5185 | | | 13742 | | | |
| | COVERAGE | 95% | | | | 81% | 100% | | | 95% | | | |
| | IDENTITY | 40% | | | | 36% | 100% | | | 41% | | | |
| PA4375 | SeqID | 10072 | | 11145 | 11516 | | 5186 | | | 13719 | | | |
| | COVERAGE | 101% | | 100% | 100% | | 100% | | | 101% | | | |
| | IDENTITY | 33% | | 45% | 28% | | 100% | | | 33% | | | |
| PA4413 | SeqID | 10030 | 10805 | 11188 | 11458 | | 5187 | 12360 | 13333 | 14077 | | | |
| | COVERAGE | 90% | 94% | 92% | 93% | | 100% | 93% | 98% | 90% | | | |
| | IDENTITY | 45% | 33% | 41% | 30% | | 100% | 33% | 32% | 44% | | | |
| PA4433 | SeqID | 10327 | 10602 | 11241 | 11289 | 11655 | 5188 | 12237 | 13356 | 13729 | | | |
| | COVERAGE | 100% | 99% | 100% | 94% | 72% | 100% | 99% | 99% | 100% | | | |
| | IDENTITY | 75% | 59% | 73% | 54% | 76% | 100% | 55% | 56% | 72% | | | |
| PA4473 | SeqID | 10463 | | 11195 | | | 5189 | | | 13986 | | | |
| | COVERAGE | 84% | | 81% | | | 100% | | | 84% | | | |
| | IDENTITY | 39% | | 37% | | | 100% | | | 39% | | | |
| PA4506 | SeqID | 10381 | 10658 | 11198 | 11314 | 11717 | 5190 | 12850 | 13248 | 13800 | | | |
| | COVERAGE | 99% | 77% | 98% | 79% | 91% | 100% | 99% | 81% | 99% | | | |
| | IDENTITY | 58% | 48% | 60% | 51% | 59% | 100% | 46% | 42% | 58% | | | |
| PA4512 | SeqID | | | | | | 5191 | | | 13815 | | | |
| | COVERAGE | 100% | | | | | | | | 99% | | | |
| | IDENTITY | | | | | | 100% | | | 57% | | | |
| PA4542 | SeqID | 10258 | 10628 | 11134 | 11489 | | 5192 | 12526 | 13421 | 14088 | | | |
| | COVERAGE | 100% | 101% | 100% | 100% | | 100% | 101% | 80% | 100% | | | |
| | IDENTITY | 71% | 47% | 70% | 49% | | 100% | 52% | 46% | 71% | | | |
| PA4576 | SeqID | | | | | | 5193 | | | | | | |
| | COVERAGE | | | | | | 100% | | | | | | |
| | IDENTITY | | | | | | 100% | | | | | | |
| PA4598 | SeqID | 10072 | | 11145 | 11516 | | 5194 | | | 13719 | | | |
| | COVERAGE | 100% | | 100% | 99% | | 100% | | | 100% | | | |
| | IDENTITY | 50% | | 29% | 28% | | 100% | | | 50% | | | |
| PA4665 | SeqID | 10143 | 10826 | 11251 | 11287 | 11675 | 5195 | 12380 | 13336 | 13979 | | | |
| | COVERAGE | 100% | 97% | 101% | 97% | 100% | 100% | 98% | 99% | 100% | | | |
| | IDENTITY | 66% | 54% | 64% | 52% | 65% | 100% | 53% | 50% | 66% | | | |
| PA4681 | SeqID | | | | | | 5196 | | | | | | |
| | COVERAGE | | | | | | 100% | | | | | | |
| | IDENTITY | | | | | | 100% | | | | | | |
| PA4709 | SeqID | | | | | | 5197 | | | | | | |
| | COVERAGE | | | | | | 100% | | | | | | |
| | IDENTITY | | | | | | 100% | | | | | | |
| PA4744 | SeqID | 10314 | | 11216 | 11501 | | 5198 | 12322 | 13663 | 13765 | | | |
| | COVERAGE | 107% | | 98% | 93% | | 100% | 78% | 91% | 107% | | | |
| | IDENTITY | 58% | | 58% | 39% | | 100% | 48% | 43% | 58% | | | |
| PA4771 | SeqID | 10387 | 11280 | 5199 | | | 13402 | 13828 | | | | | |
| | COVERAGE | 100% | | 99% | | | 100% | | 96% | 97% | | | |
| | IDENTITY | 87% | | 75% | | | 100% | | 33% | 33% | | | |
| PA4888 | SeqID | | | | | | 5200 | | | | | | |
| | COVERAGE | | | | | | 100% | | | | | | |
| | IDENTITY | | | | | | 100% | | | | | | |
| PA4942 | SeqID | 10455 | | 10972 | | | 5201 | | | 13856 | | | |
| | COVERAGE | 93% | | 91% | | | 100% | | | 95% | | | |
| | IDENTITY | 48% | | 41% | | | 100% | | | 48% | | | |
| PA4997 | SeqID | 10115 | 10619 | 10960 | 11394 | | 5202 | 12501 | 13458 | 14006 | | | |
| | COVERAGE | 86% | 82% | 97% | 83% | | 100% | 96% | 97% | 86% | | | |
| | IDENTITY | 43% | 36% | 44% | 31% | | 100% | 37% | 32% | 44% | | | |
| PA5030 | SeqID | 10165 | | | | | 5203 | | | | | | |
| | COVERAGE | 90% | | | | | 100% | | | | | | |
| | IDENTITY | 64% | | | | | 100% | | | | | | |
| PA5076 | SeqID | 10197 | 10796 | 11176 | 11383 | 11694 | 5204 | | 13292 | 14057 | | | |
| | COVERAGE | 94% | 82% | 97% | 97% | 90% | 100% | | 98% | 94% | | | |
| | IDENTITY | 29% | 33% | 27% | 26% | 29% | 100% | | 30% | 30% | | | |
| PA5088 | SeqID | | | | | | 5205 | | | | | | |
| | COVERAGE | | | | | | 100% | | | | | | |
| | IDENTITY | | | | | | 100% | | | | | | |
| PA5193 | SeqID | 10373 | | 11126 | | 11709 | 5206 | | | 13808 | | | |
| | COVERAGE | 100% | | 96% | | 77% | 100% | | | 100% | | | |
| | IDENTITY | 41% | | 39% | | 42% | 100% | | | 41% | | | |
| PA5199 | SeqID | 10375 | 10596 | | | 11711 | 5207 | | | 13810 | | | |
| | COVERAGE | 102% | 71% | | | 102% | 100% | | | 103% | | | |
| | IDENTITY | 33% | 26% | | | 34% | 100% | | | 32% | | | |
| PA5207 | SeqID | | | 11260 | 11612 | | 5208 | 12730 | | | | | |
| | COVERAGE | | | 100% | 88% | | 100% | 100% | | | | | |
| | IDENTITY | | | 54% | 39% | | 100% | 28% | | | | | |
| PA5209 | SeqID | 10302 | | | | | 5209 | | | 13758 | | | |
| | COVERAGE | 90% | | | | | 100% | | | 89% | | | |
| | IDENTITY | 29% | | | | | 100% | | | 28% | | | |
| PA5248 | SeqID | | | | | | 5210 | | | | | | |
| | COVERAGE | | | | | | 100% | | | | | | |
| | IDENTITY | | | | | | 100% | | | | | | |
| PA5299 | SeqID | | | | | | 5211 | | | | | | |
| | COVERAGE | | | | | | 100% | | | | | | |
| | IDENTITY | | | | | | 100% | | | | | | |
| PA5316 | SeqID | 10391 | | 11158 | 11327 | | 5212 | 12129 | | | | | |
| | COVERAGE | 100% | | 99% | 78% | | 100% | 73% | | | | | |
| | IDENTITY | 82% | | 79% | 39% | | 100% | 40% | | | | | |
| PA5388 | SeqID | | 10503 | | | | 5213 | | | | | | |
| | COVERAGE | | 85% | | | | 100% | | | | | | |
| | IDENTITY | | 28% | | | | 100% | | | | | | |
| PA5393 | SeqID | | | | | | 5214 | | | | | | |
| | COVERAGE | | | | | | 100% | | | | | | |
| | IDENTITY | | | | | | 100% | | | | | | |
| PA5436 | SeqID | 10330 | 10924 | 11160 | 11321 | | 5215 | 13127 | 13617 | 13885 | | | |
| | COVERAGE | 94% | 94% | 94% | 94% | | 100% | 94% | 94% | 94% | | | |
| | IDENTITY | 52% | 51% | 52% | 46% | | 100% | 55% | 54% | 52% | | | |
| PA5443 | SeqID | 10413 | 10788 | 11199 | 11452 | | 5216 | 12489 | 13643 | 13748 | | | |
| | COVERAGE | 100% | 103% | 100% | 96% | | 100% | 100% | 105% | 100% | | | |
| | IDENTITY | 64% | 38% | 56% | 35% | | 100% | 38% | 39% | 64% | | | |
| PA5490 | SeqID | | | | | | 5217 | | | | | | |
| | COVERAGE | | | | | | 100% | | | | | | |
| | IDENTITY | | | | | | 100% | | | | | | |
| PA5493 | SeqID | 10417 | 10668 | 11133 | 11609 | | 5218 | 12623 | 13236 | | | | |
| | COVERAGE | 102% | 102% | 102% | 102% | | 100% | 100% | 101% | | | | |
| | IDENTITY | 62% | 37% | 58% | 31% | | 100% | 38% | 37% | | | | |
| PA5507 | SeqID | 10119 | | | | | 5219 | | | | | | |
| | COVERAGE | 99% | | | | | 100% | | | | | | |
| | IDENTITY | 31% | | | | | 100% | | | | | | |
| PA5567 | SeqID | 10397 | 10911 | 11169 | 11450 | | 5220 | 12703 | 13338 | 13923 | | | |
| | COVERAGE | 99% | 103% | 99% | 100% | | 100% | 102% | 101% | 99% | | | |
| | IDENTITY | 67% | 39% | 64% | 33% | | 100% | 34% | 37% | 67% | | | |

TABLE VIIB

| | | | | Staphyl- |
|---|---|---|---|---|
| PathoSeq | Enterococcus | Escherichia | Pseudomonas | ococcus |
| Cluster ID | faecalis | coli | aeruginosa | aureus |
| 15 | EFA102326 | ECO101796 | PAE100280 | SAU102515 |
| 55 | EFA100151 | ECO104157 | PAE100416 | SAU100633 |
| 57 | EFA100617 | ECO102690 | PAE105434 | SAU100158 |
| 1443 | EFA100689 | ECO103692 | PAE101987 | SAU100952 |
| 1861 | EFA101412 | ECO103231 | PAE104331 | SAU101793 |
| 2286 | EFA103268 | ECO103265 | PAE104314 | SAU101756 |
| 2362 | EFA101425 | ECO100662 | PAE101537 | SAU101236 |
| 2367 | EFA101417 | ECO103226 | PAE103206 | SAU101798 |
| 2549 | EFA101410 | ECO103233 | PAE104329 | SAU101791 |
| 3816 | EFA101159 | ECO103243 | PAE104319 | SAU100546 |
| 3857 | EFA101415 | ECO103228 | PAE103204 | SAU101796 |
| 4322 | EFA101165 | ECO103237 | PAE104325 | SAU100141 |
| 4569 | EFA100955 | ECO103217 | PAE103215 | SAU101808 |
| 4948 | EFA101160 | ECO103242 | PAE104320 | SAU100547 |
| 5818 | EFA100742 | ECO103224 | PAE103208 | SAU101800 |
| 8159 | EFA101163 | ECO103239 | PAE104323 | SAU100139 |
| 8296 | EFA101164 | ECO103238 | PAE104324 | SAU100140 |
| 8316 | EFA101409 | ECO103234 | PAE104328 | SAU101790 |
| 8494 | EFA103062 | ECO103884 | PAE104311 | SAU100433 |
| 8498 | EFA101411 | ECO103232 | PAE104330 | SAU101792 |
| 8499 | EFA101416 | ECO103227 | PAE103205 | SAU101797 |
| 7 | | ECO100071 | PAE100837 | SAU102674 |
| 8 | EFA101340 | | PAE106580 | SAU100118 |
| 28 | EFA101403 | | PAE102647 | SAU100514 |
| 41 | EFA101753 | ECO100148 | | SAU101565 |
| 63 | EFA101685 | | PAE103857 | SAU100331 |
| 147 | | ECO100645 | PAE100543 | SAU100053 |
| 548 | | ECO100377 | PAE100604 | SAU100747 |
| 730 | | ECO103592 | PAE103108 | SAU100061 |
| 1721 | EFA101686 | ECO100663 | | SAU101996 |
| 1749 | EFA101477 | ECO102557 | | SAU100613 |
| 2153 | EFA102656 | ECO100184 | SAU101869 | |
| 2790 | EFA102764 | ECO100500 | SAU101578 | |
| 3164 | EFA101162 | ECO103240 | | SAU102602 |
| 3312 | EFA103174 | | PAE105008 | SAU100521 |
| 3926 | EFA100194 | ECO103220 | | SAU101806 |
| 4441 | EFA102541 | | PAE105364 | SAU101814 |
| 5685 | EFA100190 | ECO103264 | | SAU100157 |
| 7417 | EFA102788 | ECO101684 | | SAU102992 |
| 7437 | EFA102351 | ECO100084 | | SAU100056 |
| 7579 | | ECO102470 | PAE102641 | SAU100607 |
| 7726 | EFA102551 | ECO103221 | | SAU101805 |
| 7727 | EFA100978 | ECO103218 | | SAU101807 |
| 8092 | | ECO102035 | PAE102964 | SAU100794 |
| 8158 | EFA103365 | | PAE104318 | SAU102880 |
| 8161 | EFA100210 | | PAE104326 | SAU102527 |
| 8162 | EFA101414 | | PAE103203 | SAU101795 |
| 8164 | EFA100741 | ECO103223 | | SAU101801 |
| 8493 | EFA101141 | | PAE104310 | SAU100432 |
| 10185 | EFA102728 | ECO104092 | | SAU102578 |
| 35 | | ECO102870 | | SAU100497 |
| 44 | | | PAE101061 | SAU101143 |
| 54 | | | PAE100225 | SAU100123 |
| 85 | | ECO101104 | | SAU101262 |
| 184 | | | PAE104901 | SAU101366 |
| 362 | EFA102736 | | | SAU 100414 |
| 575 | EFA101790 | | | SAU100133 |
| 579 | EFA102110 | | | SAU101624 |
| 911 | | | PAE105432 | SAU102054 |
| 941 | | ECO101365 | | SAU102162 |
| 952 | EFA100615 | | | SAU100964 |
| 1084 | EFA100289 | ECO102819 | | |
| 1141 | | ECO102255 | | SAU102356 |
| 1232 | | ECO100703 | | SAU101346 |
| 1274 | | | PAE103655 | SAU102264 |
| 1337 | | ECO102562 | | SAU100567 |
| 1350 | | ECO100930 | PAE103901 | |
| 1374 | | ECO103659 | | SAU101385 |
| 1427 | EFA100394 | | | SAU100714 |
| 1535 | | ECO101207 | | SAU101561 |
| 1653 | EFA102655 | | | SAU101868 |
| 1849 | EFA100642 | | | SAU101653 |
| 1932 | EFA100919 | | | SAU101365 |
| 2156 | EFA101150 | | | SAU101271 |
| 2189 | | ECO102827 | PAE100476 | |
| 2238 | | ECO101436 | | SAU101092 |
| 2338 | EFA103038 | | | SAU100518 |
| 2411 | EFA102802 | | | SAU102246 |
| 2501 | EFA101121 | | | SAU100996 |
| 2974 | | | PAE102537 | SAU102125 |
| 3027 | | ECO103959 | | SAU200242 |
| 3239 | EFA103021 | | | SAU100300 |
| 3244 | EFA100399 | | | SAU101891 |
| 3386 | EFA100426 | | | SAU100886 |
| 3447 | EFA102915 | | | SAU102112 |
| 3460 | EFA102023 | | | SAU101399 |
| 3682 | EFA100740 | | | SAU101802 |
| 3771 | EFA101540 | | | SAU100275 |
| 4424 | EFA102542 | | | SAU101815 |
| 4654 | | ECO100488 | PAE106184 | |
| 5148 | EFA100065 | | | SAU100658 |
| 7227 | EFA100023 | | | SAU100436 |
| 7240 | | ECO103672 | | SAU101682 |
| 7278 | | | PAE101620 | SAU301370 |
| 7374 | | | PAE106765 | SAU103042 |
| 7375 | EFA102051 | | | SAU103038 |
| 7402 | | ECO103572 | PAE106044 | |
| 7419 | | ECO101686 | | SAU102693 |
| 7436 | EFA101792 | | | SAU101495 |
| 7504 | EFA101670 | | | SAU102603 |
| 7653 | EFA100397 | | | SAU100246 |
| 7660 | EFA102352 | ECO103698 | | |
| 7719 | EFA100756 | | | SAU100496 |
| 7725 | EFA100739 | | | SAU101803 |
| 8040 | EFA101736 | | | SAU101197 |
| 8058 | EFA103571 | | | SAU101242 |
| 8077 | EFA100200 | | | SAU102231 |
| 8082 | EFA101080 | | | SAU100199 |
| 8116 | EFA101963 | | | SAU101028 |
| 8122 | EFA101737 | | | SAU101198 |
| 8141 | EFA102780 | | | SAU102433 |
| 8177 | EFA103348 | | | SAU202126 |
| 8178 | EFA101022 | | | SAU102283 |
| 8181 | EFA101541 | | | SAU102909 |
| 8191 | EFA102022 | | | SAU101398 |
| 8234 | EFA103033 | | | SAU100745 |
| 8237 | EFA101682 | | | SAU101266 |
| 8238 | EFA103295 | | | SAU100963 |
| 8251 | | | PAE100662 | SAU100596 |
| 8300 | EFA101120 | | | SAU100944 |
| 8539 | EFA101339 | | | SAU101400 |
| 8610 | | ECO103661 | | SAU102298 |
| 8874 | EFA100748 | | | SAU101155 |
| 9028 | EFA103210 | | | SAU100731 |
| 9996 | EFA102338 | | | SAU100175 |
| 10234 | EFA102186 | | | SAU102933 |
| 10248 | | ECO102828 | | SAU101220 |
| 10297 | | | PAE105229 | SAU101381 |
| 10328 | EFA101079 | | | SAU101547 |
| 10345 | EFA100298 | | | SAU100659 |
| 10365 | EFA100641 | | | SAU101655 |
| 10393 | EFA103504 | | | SAU100961 |
| 10402 | EFA101833 | | | SAU100880 |
| 12426 | EFA101413 | | | SAU101794 |
| 14277 | EFA103081 | | | SAU200088 |
| 14330 | EFA101161 | | | SAU102881 |
| 14455 | EFA101424 | | | SAU101771 |
| 14520 | EFA100211 | | | SAU101789 |
| 15660 | EFA103375 | | | SAU102694 |

### Example 13

### Use of Identified Nucleic Acid Sequences as Probes

The sequences from Staphylococcus aureus, Salmonella typhimurium, Klebsiella pneumoniae, Pseudomonas aeruginosa, Enterococcus faecalis, Escherichia coli, Enterococcus faecalis, Haemophilus influenzae, Helicobacter pylori, or Salmonella typhi described herein, homologous coding nucleic acids, or homologous antisense nucleic acids can be used as probes to obtain the sequence of additional genes of interest from a second cell or microorganism. For example, probes to genes encoding potential bacterial target proteins may be hybridized to nucleic acids from other organisms including other bacteria and higher organisms, to identify homologous sequences in these other organisms. For example, the identified sequences from Staphylococcus aureus, Salmonella typhimurium, Klebsiella pneumoniae, Pseudomonas aeruginosa, Enterococcus faecalis, Escherichia coli, Enterococcus faecalis, Haemophilus influenzae, Helicobacter pylori, or Salmonella typhi, homologous coding nucleic acids, or homologous antisense nucleic acids may be used to identify homologous sequences in Anaplasma marginale, Aspergillus fumigatus, Bacillus anthracis, Bacterioides fragilis Bordetella pertussis, Burkholderia cepacia, Campylobacter jejuni, Candida albicans, Candida glabrata (also called Torulopsis glabrata), Candida tropicalis, Candida parapsilosis, Candida guilliermondii, Candida krusei, Candida kefyr (also called Candida pseudotropicalis), Candida dubliniensis, Chlamydia pneumoniae, Chlamydia trachomatus, Clostridium botulinum, Clostridium difficile, Clostridium perfringens, Coccidiodes immitis, Corynebacterium diptheriae, Cryptococcus neoformans, Enterobacter cloacae, Enterococcus faecalis, Enterococcus faecium, Escherichia coli, Haemophilus influenzae, Helicobacter pylori, Histoplasma capsulatum, Klebsiella pneumoniae, Listeria monocytogenes, Mycobacterium leprae, Mycobacterium tuberculosis, Neisseria gonorrhoeae, Neisseria meningitidis, Nocardia asteroides, Pasteurella haemolytica, Pasteurella multocida, Pneumocystis carinii, Proteus vulgaris, Pseudomonas aeruginosa, Salmonella bongori, Salmonella cholerasuis, Salmonella enterica, Salmonella paratyphi, Salmonella typhi, Salmonella typhimurium, Staphylococcus aureus, Listeria monocytogenes, Moxarella catarrhalis, Shigella boydii, Shigella dysenteriae, Shigella flexneri, Shigella sonnei, Staphylococcus epidermidis, Streptococcus pneumoniae, Streptococcus mutans, Treponema pallidum, Yersinia enterocolitica, Yersinia pestis and any species falling within the genera of any of the above species. In some embodiments of the present invention, the nucleic acids from Staphylococcus aureus, Salmonella typhimurium, Klebsiella pneumoniae, Pseudomonas aeruginosa, Enterococcus faecalis, Escherichia coli, Enterococcus faecalis, Haemophilus influenzae, Helicobacter pylori, or Salmonella typhi described herein, homologous coding nucleic acids, or homologous antisense nucleic acids may be used to identify homologous nucleic acids from a heterologous organism other than E. coli.

Hybridization between the nucleic acids from Staphylococcus aureus, Salmonella typhimurium, Klebsiella pneumoniae, Pseudomonas aeruginosa, Enterococcus faecalis, Escherichia coli, Enterococcus faecalis, Haemophilus influenzae, Helicobacter pylori, or Salmonella typhi described herein, homologous coding nucleic acids, or homologous antisense nucleic acids and nucleic acids from humans might indicate that the protein encoded by the gene to which the probe corresponds is found in humans and therefore not necessarily an optimal drug target. Alternatively, the gene can be conserved only in bacteria and therefore would be a good drug target for a broad spectrum antibiotic or antimicrobial. These probes can also be used in a known manner to isolate homologous nucleic acids from Staphylococcus, Salmonella, Klebsiella, Pseudomonas, Enterococcus or other cells or microorganisms, e.g. by screening a genomic or cDNA library.

Probes derived from the nucleic acid sequences from Staphylococcus aureus, Salmonella typhimurium, Klebsiella pneumoniae, Pseudomonas aeruginosa, Enterococcus faecalis, Escherichia coli Enterococcus faecalis, Haemophilus influenzae, Helicobacter pylori, or Salmonella typhi described herein, homologous coding nucleic acids, or homologous antisense nucleic acids, or portions thereof, can be labeled with detectable labels familiar to those skilled in the art, including radioisotopes and non-radioactive labels, to provide a detectable probe. The detectable probe can be single stranded or double stranded and can be made using techniques known in the art, including in vitro transcription, nick translation, or kinase reactions. A nucleic acid sample containing a sequence capable of hybridizing to the labeled probe is contacted with the labeled probe. If the nucleic acid in the sample is double stranded, it can be denatured prior to contacting the probe. In some applications, the nucleic acid sample can be immobilized on a surface such as a nitrocellulose or nylon membrane. The nucleic acid sample can comprise nucleic acids obtained from a variety of sources, including genomic DNA, cDNA libraries, RNA, or tissue samples.

Procedures used to detect the presence of nucleic acids capable of hybridizing to the detectable probe include well known techniques such as Southern blotting, Northern blotting, dot blotting, colony hybridization, and plaque hybridization. In some applications, the nucleic acid capable of hybridizing to the labeled probe can be cloned into vectors such as expression vectors, sequencing vectors, or in vitro transcription vectors to facilitate the characterization and expression of the hybridizing nucleic acids in the sample. For example, such techniques can be used to isolate, purify and clone sequences from a genomic library, made from a variety of bacterial species, which are capable of hybridizing to probes made from the sequences identified in Examples 5 and 6.

### Example 14

### Preparation of PCR Primers and Amplification of DNA

The identified Staphylococcus aureus, Salmonella typhimurium, Klebsiella pneumoniae, Pseudomonas aeruginosa, Enterococcus faecalis, Escherichia coli, Enterococcus faecalis, Haemophilus influenzae, Helicobacter pylori, or Salmonella typhi genes corresponding directly to or located within the operon of nucleic acid sequences required for proliferation, homologous coding nucleic acids, or homologous antisense nucleic acids or portions thereof can be used to prepare PCR primers for a variety of applications, including the identification or isolation of homologous sequences from other species. For example, the Staphylococcus aureus, Salmonella typhimurium, Klebsiella pneumoniae, Pseudomonas aeruginosa, Enterococcus faecalis, Escherichia coli, Enterococcus faecalis, Haemophilus influenzae, Helicobacter pylori, or Salmonella typhi genes may be used to prepare PCR primers to identify or isolate homologous sequences from Anaplasma marginale, Aspergillus fumigatus, Bacillus anthracis, Bacterioides fragilis Bordetella pertussis, Burkholderia cepacia, Campylobacter jejuni, Candida albicans, Candida glabrata (also called Torulopsis glabrata), Candida tropicalis, Candida parapsilosis, Candida guilliermondii, Candida krusei, Candida kefyr (also called Candida pseudotropicalis), Candida dubliniensis, Chlamydia pneumoniae, Chlamydia trachomatus, Clostridium botulinum, Clostridium difficile, Clostridium perfringens, Coccidiodes immitis, Corynebacterium diptheriae, Cryptococcus neoformans, Enterobacter cloacae, Enterococcus faecalis, Enterococcus faecium, Escherichia coli, Haemophilus influenzae, Helicobacter pylori, Histoplasma capsulatum, Klebsiella pneumoniae, Listeria monocytogenes, Mycobacterium leprae, Mycobacterium tuberculosis, Neisseria gonorrhoeae, Neisseria meningitidis; Nocardia asteroides, Pasteurella haemolytica, Pasteurella multocida, Pneumocystis carinii, Proteus vulgaris, Pseudomonas aeruginosa, Salmonella bongori, Salmonella cholerasuis, Salmonella enterica, Salmonella paratyphi, Salmonella typhi, Salmonella typhimurium, Staphylococcus aureus, Listeria monocytogenes, Moxarella catarrhalis, Shigella boydii, Shigella dysenteriae, Shigella flexneri, Shigella sonnei, Staphylococcus epidermidis, Streptococcus pneumoniae, Streptococcus mutans, Treponema pallidum, Yersinia enterocolitica, Yersinia pestis or any species falling within the genera of any of the above species. In some embodiments of the present invention, the PCR primers may be used to identify or isolate homologous nucleic acids from an organism other than E. coli.

The identified or isolated nucleic acids obtained using the PCR primers may contain part or all of the homologous nucleic acids. Because homologous nucleic acids are related but not identical in sequence, those skilled in the art will often employ degenerate sequence PCR primers. Such degenerate sequence primers are designed based on sequence regions that are either known to be conserved or suspected to be conserved such as conserved coding regions. The successful production of a PCR product using degenerate probes generated from the sequences identified herein would indicate the presence of a homologous gene sequence in the species being screened. The PCR primers are at least 10 nucleotides, and preferably at least 20 nucleotides in length. More preferably, the PCR primers are at least 20-30 nucleotides in length. In some embodiments, the PCR primers can be more than 30 nucleotides in length. It is preferred that the primer pairs have approximately the same G/C ratio, so that melting temperatures are approximately the same. A variety of PCR techniques are familiar to those skilled in the art. For a review of PCR technology, see Molecular Cloning to Genetic Engineering White, B. A. Ed. in Methods in Molecular Biology 67: Humana Press, Totowa 1997. When the entire coding sequence of the target gene is known, the 5′ and 3′ regions of the target gene can be used as the sequence source for PCR probe generation. In each of these PCR procedures, PCR primers on either side of the nucleic acid sequences to be amplified are added to a suitably prepared nucleic acid sample along with dNTPs and a thermostable polymerase such as Taq polymerase, Pfu polymerase, or Vent polymerase. The nucleic acid in the sample is denatured and the PCR primers are specifically hybridized to complementary nucleic acid sequences in the sample. The hybridized primers are extended. Thereafter, another cycle of denaturation, hybridization, and extension is initiated. The cycles are repeated multiple times to produce an amplified fragment containing the nucleic acid sequence between the primer sites.

### Example 15

### Inverse PCR

The technique of inverse polymerase chain reaction can be used to extend the known nucleic acid sequence identified in Examples 5 and 6. The inverse PCR reaction is described generally by Ochman et al., in Ch. 10 of PCR Technology: Principles and Applications for DNA Amplification, (Henry A. Erlich, Ed.) W.H. Freeman and Co. (1992). Traditional PCR requires two primers that are used to prime the synthesis of complementary strands of DNA. In inverse PCR, only a core sequence need be known.

Using the sequences identified as relevant from the techniques taught in Examples 5 and 6 and applied to other species of bacteria, a subset of nucleic sequences are identified that correspond to genes or operons that are required for bacterial proliferation. In species for which a genome sequence is not known, the technique of inverse PCR provides a method for obtaining the gene in order to determine the sequence or to place the probe sequences in full context to the target sequence to which the identified nucleic acid sequence binds.

To practice this technique, the genome of the target organism is digested with an appropriate restriction enzyme so as to create fragments of nucleic acid that contain the identified sequence as well as unknown sequences that flank the identified sequence. These fragments are then circularized and become the template for the PCR reaction. PCR primers are designed in accordance with the teachings of Example 15 and directed to the ends of the identified sequence. The primers direct nucleic acid synthesis away from the known sequence and toward the unknown sequence contained within the circularized template. After the PCR reaction is complete, the resulting PCR products can be sequenced so as to extend the sequence of the identified gene past the core sequence of the identified exogenous nucleic acid sequence identified. In this manner, the full sequence of each novel gene can be identified. Additionally the sequences of adjacent coding and noncoding regions can be identified.

### Example 16

### Identification of Genes Required for Escherichia coli Proliferation

Genes required for proliferation in Escherichia coli are identified according to the methods described above.

### Example 17

### Identification of Genes Required for Neisseria gonorrhoeae Proliferation

Genes required for proliferation in Neisseria gonorrhoeae are identified according to the methods described above.

### Example 18

### Identification of Genes Required for Salmonella enterica Proliferation

Genes required for proliferation in Salmonella enterica are identified according to the methods described above.

### Example 19

### Identification of Genes Required for Enterococcus faecium Proliferation

Genes required for proliferation in Enterococcus faecium are identified according to the methods described above.

### Example 20

### Identification of Genes Required for Haemophilus influenzae Proliferation

Genes required for proliferation in Haemophilus influenzae are identified according to the methods described above.

### Example 21

### Identification of Genes Required for Aspergillus fumigatus Proliferation

Genes required for proliferation in Aspergillus fumigatus are identified according to the methods described above.

### Example 22

### Identification of Genes Required for Helicobacter pylori Proliferation

Genes required for proliferation in Helicobacter pylori are identified according to the methods described above.

### Example 23

### Identification of Genes Required for Mycoplasma pneumoniae Proliferation

Genes required for proliferation in Mycoplasma pneumoniae are identified according to the methods described above.

### Example 24

### Identification of Genes Required for Plasmodium ovale Proliferation

Genes required for proliferation in Plasmodium ovale are identified according to the methods described above.

### Example 25

### Identification of Genes Required for Entamoeba histolytica Proliferation

Genes required for proliferation in Entamoeba histolytica are identified according to the methods described above.

### Example 26

### Identification of Genes Required for Candida albicans Proliferation

Genes required for proliferation in Candida albicans are identified according to the methods described above.

### Example 27

### Identification of Genes Required for Histoplasma capsulatum Proliferation

Genes required for proliferation in Histoplasma capsulatum are identified according to the methods described above.

### Example 28

### Identification of Genes Required for Salmonella typhi Proliferation

Genes required for proliferation in Salmonella typhi are identified according to the methods described above.

### Example 29

### Identification of Genes Required for Salmonella paratyphi Proliferation

Genes required for proliferation in Salmonella paratyphi are identified according to the methods described above.

### Example 30

### Identification of Genes Required for Salmonella cholerasuis Proliferation

Genes required for proliferation in Salmonella cholerasuis are identified according to the methods described above.

### Example 31

### Identification of Genes Required for Staphylococcus epidermis Proliferation

Genes required for proliferation in Staphylococcus epidermis are identified according to the methods described above.

### Example 32

### Identification of Genes Required for Mycobacterium tuberculosis Proliferation

Genes required for proliferation in Mycobacterium tuberculosis are identified according to the methods described above.

### Example 33

### Identification of Genes Required for Mycobacterium leprae Proliferation

Genes required for proliferation in Mycobacterium leprae are identified according to the methods described above.

### Example 34

### Identification of Genes Required for Treponema pallidum Proliferation

Genes required for proliferation in Treponema pallidum are identified according to the methods described above.

### Example 35

### Identification of Genes Required for Bacillus anthracis Proliferation

Genes required for proliferation in Bacillus anthracis are identified according to the methods described above.

### Example 36

### Identification of Genes Required for Yersinia pestis Proliferation

Genes required for proliferation in Yersinia pestis are identified according to the methods described above.

### Example 37

### Identification of Genes Required for Clostridium botulinum Proliferation

Genes required for proliferation in Clostridium botulinum are identified according to the methods described above.

### Example 38

### Identification of Genes Required for Campylobacter jejuni Proliferation

Genes required for proliferation in Campylobacter jejuni are identified according to the methods described above.

### Example 39

### Identification of Genes Required for Chlamydia trachomatis Proliferation

Genes required for proliferation in Chlamydia trachomatis are identified according to the methods described above.

### Example 40

### Identification of Genes Required for Staphylococcus aureus Proliferation

Genes required for proliferation in Staphylococcus aureus are identified according to the methods described above.

### Example 41

### Identification of Genes Required for Salmonella typhimurium Proliferation

Genes required for proliferation in Salmonella typhimurium are identified according to the methods described above.

### Example 42

### Identification of Genes Required for Klebsiella Pneumoniae Proliferation

Genes required for proliferation in Klebsiella Pneumoniae are identified according to the methods described above.

### Example 43

### Identification of Genes Required for Pseudomonas aeruginosa Proliferation

Genes required for proliferation in Pseudomonas aeruginosa are identified according to the methods described above.

### Example 44

### Identification of Genes Required for Enterococcus faecalis Proliferation

Genes required for proliferation in Enterococcus faecalis are identified according to the methods described above.

Use of Isolated Exogenous Nucleic Acid Fragments as Antisense Antibiotics

In addition to using the identified sequences to enable screening of molecule libraries to identify compounds useful to identify antibiotics, antisense nucleic acids complementary to the proliferation-required sequences or portions thereof, antisense nucleic acids complementary to homologous coding nucleic acids, or homologous antisense nucleic acids can be used as therapeutic agents. Specifically, the proliferation-required sequences or homolgous coding nucleic acids, or portions therof, in an antisense orientation or homologous antisense nucleic acids can be provided to an individual to inhibit the translation of a bacterial target gene or the processing, folding, or assembly into a protein/RNA complex of a nontranslated RNA.

### Example 45

### Generation of Antisense Therapeutics from Identified Exogenous Sequences

Antisense nucleic acids complementary to the proliferation-required sequences described herein, or portions thereof, antisense nucleic acids complementary to homologous coding nucleic acids, or portions thereof, or homologous antisense nucleic acids or portions thereof can be used as antisense therapeutics for the treatment of bacterial infections or simply for inhibition of bacterial growth in vitro or in vivo. For example, the antisense therapeutics may be used to treat bacterial infections caused by Staphylococcus aureus, Salmonella typhimurium, Klebsiella pneumoniae, Pseudomonas aeruginosa, Enterococcus faecalis, Escherichia coli, Enterococcus faecalis, Haemophilus influenzae, Helicobacter pylori, or Salmonella typhi or to inhibit the growth of these organisms. The antisense therapeutics may also be used to treat infections caused by or to inhibit the growth of Anaplasma marginale, Aspergillus fumigatus, Bacillus anthracis, Bacterioides fragilis Bordetella pertussis, Burkholderia cepacia, Campylobacter jejuni, Candida albicans, Candida glabrata (also called Torulopsis glabrata), Candida tropicalis, Candida parapsilosis, Candida guilliermondii, Candida krusei, Candida kefyr (also called Candida pseudotropicalis), Candida dubliniensis, Chlamydia pneumoniae, Chlamydia trachomatus, Clostridium botulinum, Clostridium difficile, Clostridium perfringens, Coccidiodes immitis, Corynebacterium diptheriae, Cryptococcus neoformans, Enterobacter cloacae, Enterococcus faecalis, Enterococcus faecium, Escherichia colt, Haemophilus influenzae, Helicobacter pylori, Histoplasma capsulatum, Klebsiella pneumoniae, Listeria monocytogenes, Mycobacterium leprae, Mycobacterium tuberculosis, Neisseria gonorrhoeae, Neisseria meningitidis, Nocardia asteroides, Pasteurella haemolytica, Pasteurella multocida, Pneumocystis carinii, Proteus vulgaris, Pseudomonas aeruginosa, Salmonella bongori, Salmonella cholerasuis, Salmonella enterica, Salmonella paratyphi, Salmonella typhi, Salmonella typhimurium, Staphylococcus aureus, Listeria monocytogenes, Moxarella catarrhalis, Shigella boydii, Shigella dysenteriae, Shigella flexneri, Shigella sonnei, Staphylococcus epidermidis, Streptococcus pneumoniae, Streptococcus mutans, Treponema pallidum, Yersinia enterocolitica, Yersinia pestis or any species falling within the genera of any of the above species. In some embodiments of the present invention, the antisense therapuetics may be used to treat infection by or inhibit the growth of an organism other than E. coli.

The therapy exploits the biological process in cells where genes are transcribed into messenger RNA (mRNA) that is then translated into proteins. Antisense RNA technology contemplates the use of antisense nucleic acids, including antisense oligonucleotides, complementary to a target gene that will bind to its target nucleic acid and decrease or inhibit the expression of the target gene. For example, the antisense nucleic acid may inhibit the translation or transcription of the target nucleic acid. In one embodiment, antisense oligonucleotides can be used to treat and control a bacterial infection of a cell culture containing a population of desired cells contaminated with bacteria. In another embodiment, the antisense oligonucleotides can be used to treat an organism with a bacterial infection.

Antisense oligonucleotides can be synthesized from any of the sequences of the present invention using methods well known in the art. In a preferred embodiment, antisense oligonucleotides are synthesized using artificial means. Uhlmann & Peymann, Chemical Rev. 90:543-584 (1990) review antisense oligonucleotide technology in detail. Modified or unmodified antisense oligonucleotides can be used as therapeutic agents. Modified antisense oligonucleotides are preferred. Modification of the phosphate backbones of the antisense oligonucleotides can be achieved by substituting the intemucleotide phosphate residues with methylphosphonates, phosphorothioates, phosphoramidates, and phosphate esters. Nonphosphate internucleotide analogs such as siloxane bridges, carbonate brides, thioester bridges, as well as many others known in the art may also be used. The preparation of certain antisense oligonucleotides with modified internucleotide linkages is described in U.S. Pat. No. 5,142,047, hereby incorporated by reference.

Modifications to the nucleoside units of the antisense oligonucleotides are also contemplated. These modifications can increase the half-life and increase cellular rates of uptake for the oligonucleotides in vivo. For example, α-anomeric nucleotide units and modified nucleotides such as 1,2-dideoxy-d-ribofaranose, 1,2-dideoxy-1-phenylribofuranose, and N⁴, N⁴-ethano-5-methyl-cytosine are contemplated for use in the present invention.

An additional form of modified antisense molecules is found in peptide nucleic acids. Peptide nucleic acids (PNA) have been developed to hybridize to single and double stranded nucleic acids. PNA are nucleic acid analogs in which the entire deoxyribose-phosphate backbone has been exchanged with a chemically different, but structurally homologous, polyamide (peptide) backbone containing 2-aminoethyl glycine units. Unlike DNA, which is highly negatively charged, the PNA backbone is neutral. Therefore, there is much less repulsive energy between complementary strands in a PNA-DNA hybrid than in the comparable DNA-DNA hybrid, and consequently they are much more stable. PNA can hybridize to DNA in either a Watson/Crick or Hoogsteen fashion (Demidov et al., Proc. Natl. Acad. Sci. U.S.A. 92:2637-2641, 1995; Egholm, Nature 365:566-568, 1993; Nielsen et al., Science 254:1497-1500, 1991; Dueholm et al., New J. Chem. 21:19-31, 1997).

Molecules called PNA “clamps” have been synthesized which have two identical PNA sequences joined by a flexible hairpin linker containing three 8-amino-3,6-dioxaoctanoic acid units. When a PNA clamp is mixed with a complementary homopurine or homopyrimidine DNA target sequence, a PNA-DNA-PNA triplex hybrid can form which has been shown to be extremely stable (Bentin et al., Biochemistry 35:8863-8869, 1996; Egholm et al., Nucleic Acids Res. 23:217-222, 1995; Griffith et al., J. Am. Chem. Soc. 117:831-832, 1995).

The sequence-specific and high affinity duplex and triplex binding of PNA have been extensively described (Nielsen et al., Science 254:1497-1500, 1991; Egholm et al., J. Am. Chem. Soc. 114:9677-9678, 1992; Egholm et al., Nature 365:566-568, 1993; Almarsson et al., Proc. Natl. Acad. Sci. USA. 90:9542-9546, 1993; Demidov et al., Proc. Natl. Acad. Sci. USA. 92:2637-2641, 1995). They have also been shown to be resistant to nuclease and protease digestion (Demidov et al., Biochem. Pharm. 48:1010-1313, 1994). PNA has been used to inhibit gene expression (Hanvey et al., Science 258:1481-1485,1992; Nielsen et al., Nucl. Acids. Res., 21:197-200, 1993; Nielsen et al., Gene 149:139-145, 1994; Good & Nielsen, Science, 95: 2073-2076, 1998; all of which are hereby incorporated by reference), to block restriction enzyme activity (Nielsen et al., supra., 1993), to act as an artificial transcription promoter (Mollegaard, Proc. Natl. Acad Sci. U.S.A. 91:3892-3895, 1994) and as a pseudo restriction endonuclease (Demidov et al., Nucl. Acids. Res. 21:2103-2107, 1993). Recently, PNA has also been shown to have antiviral and antitumoral activity mediated through an antisense mechanism (Norton, Nature Biotechnol., 14:615-619, 1996; Hirschman et al., J. Investig. Med. 44:347-351, 1996). PNAs have been linked to various peptides in order to promote PNA entry into cells (Basu et al., Bioconj. Chem. 8:481-488, 1997; Pardridge et al., Proc. Natl. Acad. Sci. U.S.A. 92:5592-5596, 1995).

The antisense oligonucleotides contemplated by the present invention can be administered by direct application of oligonucleotides to a target using standard techniques well known in the art. The antisense oligonucleotides can be generated within the target using a plasmid, or a phage. Alternatively, the antisense nucleic acid may be expressed from a sequence in the chromosome of the target cell. For example, a promoter may be introduced into the chromosome of the target cell near the target gene such that the promoter directs the transcription of the antisense nucleic acid. Alternatively, a nucleic acid containing the antisense sequence operably linked to a promoter may be introduced into the chromosome of the target cell. It is further contemplated that the antisense oligonucleotides are incorporated in a ribozyme sequence to enable the antisense to specifically bind and cleave its target mRNA. For technical applications of ribozyme and antisense oligonucleotides see Rossi et al., Pharmacol. Ther. 50(2):245-254, (1991), which is hereby incorporated by reference. The present invention also contemplates using a retron to introduce an antisense oligonucleotide to a cell. Retron technology is exemplified by U.S. Pat. No. 5,405,775, which is hereby incorporated by reference. Antisense oligonucleotides can also be delivered using liposomes or by electroporation techniques which are well known in the art.

The antisense nucleic acids described above can also be used to design antibiotic compounds comprising nucleic acids which function by intracellular triple helix formation. Triple helix oligonucleotides are used to inhibit transcription from a genome. The antisense nucleic acids can be used to inhibit cell or microorganism gene expression in individuals infected with such microorganisms or containing such cells. Traditionally, homopurine sequences were considered the most useful for triple helix strategies. However, homopyrimidine sequences can also inhibit gene expression. Such homopyrimidine oligonucleotides bind to the major groove at homopurine:homopyrimidine sequences. Thus, both types of sequences based on the sequences from Staphylococcus aureus, Salmonella typhimurium, Klebsiella pneumoniae, Pseudomonas aeruginosa, Enterococcus faecalis, Escherichia colt, Enterococcus faecalis, Haemophilus influenzae, Helicobacter pylori, or Salmonella typhi or homologous nucleic acids that are required for proliferation are contemplated for use as antibiotic compound templates.

The antisense nucleic acids, such as antisense oligonucleotides, which are complementary to the proliferation-required nucleic acids from Staphylococcus aureus, Salmonella typhimurium, Klebsiella pneumoniae, Pseudomonas aeruginosa, Enterococcus faecalis, Escherichia coli, Enterococcus faecalis, Haemophilus influenzae, Helicobacter pylori, or Salmonella typhi or to homologous coding nucleic acids, or portions thereof, may be used to induce bacterial cell death or at least bacterial stasis by inhibiting target nucleic acid transcription or translation. Antisense oligonucleotides complementary to about 8 to 40 nucleotides of the proliferation-required nucleic acids described herein or homologous coding nucleic acids have sufficient complementarity to form a duplex with the target sequence under physiological conditions.

To kill bacterial cells or inhibit their growth, the antisense oligonucleotides are applied to the bacteria or to the target cells under conditions that facilitate their uptake. These conditions include sufficient incubation times of cells and oligonucleotides so that the antisense oligonucleotides are taken up by the cells. In one embodiment, an incubation period of 7-10 days is sufficient to kill bacteria in a sample. An optimum concentration of antisense oligonucleotides is selected for use.

The concentration of antisense oligonucleotides to be used can vary depending on the type of bacteria sought to be controlled, the nature of the antisense oligonucleotide to be used, and the relative toxicity of the antisense oligonucleotide to the desired cells in the treated culture. Antisense oligonucleotides can be introduced to cell samples at a number of different concentrations preferably between 1×10⁻¹⁰M to 1×10⁻⁴M. Once the minimum concentration that can adequately control gene expression is identified, the optimized dose is translated into a dosage suitable for use in vivo. For example, an inhibiting concentration in culture of 1×10⁻⁷ translates into a dose of approximately 0.6 mg/kg body weight. Levels of oligonucleotide approaching 100 mg/kg body weight or higher may be possible after testing the toxicity of the oligonucleotide in laboratory animals. It is additionally contemplated that cells from the subject are removed, treated with the antisense oligonucleotide, and reintroduced into the subject. This range is merely illustrative and one of skill in the art are able to determine the optimal concentration to be used in a given case.

After the bacterial cells have been killed or controlled in a desired culture, the desired cell population may be used for other purposes.

### Example 46

### Use of Antisense Oligonucleotides to Treat Contaminated Cell Cultures

The following example demonstrates the ability of an Staphylococcus aureus, Salmonella typhimurium, Klebsiella pneumoniae, Pseudomonas aeruginosa, Enterococcus faecalis, Escherichia coli, Enterococcus faecalis, Haemophilus influenzae, Helicobacter pylori, or Salmonella typhi antisense oligonucleotide or an antisense oligonucleotide complementary to a homologous coding nucleic acid, or portions thereof, to act as a bacteriocidal or bacteriostatic agent to treat a contaminated cell culture system. The application of the antisense oligonucleotides of the present invention are thought to inhibit the translation of bacterial gene products required for proliferation. The antisense nucleic acids may also inhibit the transcription, folding or processing of the target RNA.

In one embodiment of the present invention, the antisense oligonucleotide may comprise a phosphorothioate modified nucleic acid comprising at least about 15, at least about 20, at least about 25, at least about 30, at least about 35, at least about 40, or more than 40 consecutive nucleotides of an antisense nucleic acid listed in Table IA. A sense oligodeoxynucleotide complementary to the antisense sequence is synthesized and used as a control. The oligonucleotides are synthesized and purified according to the procedures of Matsukura, et al., Gene 72:343 (1988). The test oligonucleotides are dissolved in a small volume of autoclaved water and added to culture medium to make a 100 micromolar stock solution.

Human bone marrow cells are obtained from the peripheral blood of two patients and cultured according standard procedures well known in the art. The culture is contaminated with Staphylococcus aureus, Salmonella typhimurium, Klebsiella pneumoniae, Pseudomonas aeruginosa, Enterococcus faecalis, Escherichia coli, Enterococcus faecalis, Haemophilus influenzae, Helicobacter pylori, or Salmonella typhi or an organism containing a homologous nucleic acid and incubated at 37° C. overnight to establish bacterial infection.

The control and antisense oligonucleotide containing solutions are added to the contaminated cultures and monitored for bacterial growth. After a 10 hour incubation of culture and oligonucleotides, samples from the control and experimental cultures are drawn and analyzed for the translation of the target bacterial gene using standard microbiological techniques well known in the art. The target Staphylococcus aureus, Salmonella typhimurium, Klebsiella pneumoniae, Pseudomonas aeruginosa, Enterococcus faecalis, Escherichia coli Enterococcus faecalis, Haemophilus influenzae, Helicobacter pylori, or Salmonella typhi gene or an organism containing the homologous coding nucleic acid is found to be translated in the control culture treated with the control oligonucleotide, however, translation of the target gene in the experimental culture treated with the antisense oligonucleotide of the present invention is not detected or reduced, indicating that the culture is no longer contaminated or is contaminated at a reduced level.

### Example 47

### Use of Antisense Oligonucleotides to Treat Infections

A subject suffering from a Staphylococcus aureus, Salmonella typhimurium, Klebsiella pneumoniae, Pseudomonas aeruginosa, Enterococcus faecalis, Escherichia coli, Enterococcus faecalis, Haemophilus influenzae, Helicobacter pylori, or Salmonella typhi infection or an infection with an organism containing a homologous coding nucleic acid is treated with the antisense oligonucleotide preparation above. The antisense oligonucleotide is provided in a pharmaceutically acceptable carrier at a concentration effective to inhibit the transcription or translation of the target nucleic acid. The present subject is treated with a concentration of antisense oligonucleotide sufficient to achieve a blood concentration of about 0.1-100 micromolar. The patient receives daily injections of antisense oligonucleotide to maintain this concentration for a period of 1 week. At the end of the week a blood sample is drawn and analyzed for the presence or absence of the organism using standard techniques well known in the art. There is no detectable evidence of Staphylococcus aureus, Salmonella typhimurium, Klebsiella pneumoniae, Pseudomonas aeruginosa, Enterococcus faecalis, Escherichia coli Enterococcus faecalis, Haemophilus influenzae, Helicobacter pylori, or Salmonella typhi or an organim containing a homologous coding nucleic acid and the treatment is terminated.

Antisense nucleic acids complementary to a homologous coding nucleic acid or a portion thereof may be used in the preceding method to treat individuals infected with an organism containing the homologous coding nucleic acid.

### Example 48

### Preparation and Use of Triple Helix Forming Oligonucleotides

The sequences of proliferation-required nucleic acids, homologous coding nucleic acids, or homologous antisense nucleic acids are scanned to identify 10-mer to 20-mer homopyrimidine or homopurine stretches that could be used in triple-helix based strategies for inhibiting gene expression. Following identification of candidate homopyrimidine or homopurine stretches, their efficiency in inhibiting gene expression is assessed by introducing varying amounts of oligonucleotides containing the candidate sequences into a population of bacterial cells that normally express the target gene. The oligonucleotides may be prepared on an oligonucleotide synthesizer or they may be purchased commercially from a company specializing in custom oligonucleotide synthesis.

The oligonucleotides can be introduced into the cells using a variety of methods known to those skilled in the art, including but not limited to calcium phosphate precipitation, DEAE-Dextran, electroporation, liposome-mediated transfection or native uptake.

Treated cells are monitored for a reduction in proliferation using techniques such as monitoring growth levels as compared to untreated cells using optical density measurements. The oligonucleotides that are effective in inhibiting gene expression in cultured cells can then be introduced in vivo using the techniques well known in that art at a dosage level shown to be effective.

In some embodiments, the natural (beta) anomers of the oligonucleotide units can be replaced with alpha anomers to render the oligonucleotide more resistant to nucleases. Further, an intercalating agent such as ethidium bromide, or the like, can be attached to the 3′ end of the alpha oligonucleotide to stabilize the triple helix. For information on the generation of oligonucleotides suitable for triple helix formation see Griffin et al. (Science 245:967-971 (1989), which is hereby incorporated by this reference).

### Example 49

### Identification of Bacterial Strains from Isolated Specimens by PCR

Classical bacteriological methods for the detection of various bacterial species are time consuming and costly. These methods include growing the bacteria isolated from a subject in specialized medium, cultivation on selective agar medium, followed by a set of confirmation assays that can take from 8 to 10 days or longer to complete. Use of the identified sequences of the present invention provides a method to dramatically reduce the time necessary to detect and identify specific bacterial species present in a sample.

In one exemplary method, bacteria are grown in enriched medium and DNA samples are isolated from specimens of, for example, blood, urine, stool, saliva or central nervous system fluid by conventional methods. A panel of PCR primers based on identified sequences unique to various species or types of cells or microorganisms are then utilized in accordance with Example 12 to amplify DNA of approximately 100-200 nucleotides in length from the specimen. A separate PCR reaction is set up for each pair of PCR primers and after the PCR reaction is complete, the reaction mixtures are assayed for the presence of PCR product. The presence or absence of bacteria from the species to which the PCR primer pairs belong is determined by the presence or absence of a PCR product in the various test PCR reaction tubes.

Although the PCR reaction is used to assay the isolated sample for the presence of various bacterial species, other assays such as the Southern blot hybridization are also contemplated.

Compounds which inhibit the activity or reduce the amount of gene products required for proliferation may be identified using rational drug design. These methods may be used with the proliferation-required polypeptides described herein or homologous polypeptides. In such methods, the structure of the gene product is determined using methods such as x-ray crystallography, NMR, or computer modelling. Compounds are screened to identify those which have a structure which allows them to interact with the gene product. In some embodiments, the compounds are screened to identify those which have structures which allow them to interact with regions of the gene product which are important for its activity. For example, the compounds may be screened to identify those which have structures which allow them to bind to the active site of the gene product to inhibit its activity. For example, the compound may be a suicide substrate which binds to the active site with high affinity, thereby preventing the gene product from acting on its natural substrate. Alternatively, the compound may bind to a region of the gene product which is involved in complex formation with other biomolecules. In such instances, the activity of the gene product is inhibited by blocking the interaction between the gene product and other members of the complex.

Thus, one embodiment of the present invention comprises a method of using a crystal of the gene products of the present invention and/or a dataset comprising the three-dimensional coordinates obtained from the crystal in a drug-screening assay. The present invention also includes agents (modulators or drugs) that are identified by the methods of the present invention, along with the method of using agents (modulators or drugs) identified by a method of the present invention, for inhibiting the activity of or modulating the amount of an essential gene product. The present invention also includes crystals comprising the gene products of the present invention or portions thereof.

In some embodiments of the present invention, the three-dimensional structure of the polypeptides required for proliferation is determined using X-ray crystallography or NMR. The coordinates of the determined structure are used in computer-assisted modeling programs to identify compounds that bind to and/or modulate the activity or amount of the encoded polypeptide. The method may include the following steps: 1) the generation of high-purity crystals of the encoded recombinant (or endogenous) polypeptide for analysis; 2) determination of the three-dimensional structure of the polypeptide; and, 3) the use of computer-assisted “docking” programs to analyze the molecular interaction of compound structure and the polypeptide (i.e., drug screening).

General methods for performing each of the above steps are described below and are also well known to those of skill in the art. Any method known to those of skill in the art, including those described herein, may be employed for generating the three-dimensional structure for each identified essential gene product and its use in the drug-screening assays.

Crystals of the gene products required for proliferation may be obtained as follows. Under certain conditions, molecules condense from solution into a highly-ordered crystalline lattice, which is defined by a unit cell, the smallest repeating volume of the crystalline array. The contents of such a cell can interact with and diffract certain electromagnetic and particle waves (e.g., X-rays, neutron beams, electron beams etc.). Due to the symmetry of the lattice, the diffracted waves interact to create a diffraction pattern. By measuring the diffraction pattern, crystallographers are able to reconstruct the three-dimensional structure of the atoms in the crystal.

Any method known to those of skill in the art, including those set forth below, may be employed to prepare high-purity crystals. For example, crystals of the product of the identified essential gene can be grown by a number of techniques including batch crystallization, vapor diffusion (either by sitting drop or hanging drop) and by microdialysis. Seeding of the crystals in some instances is required to obtain X-ray quality crystals. Standard micro and/or macro seeding of crystals may therefore be used. Exemplified below is the hanging-drop vapor diffusion procedure. Hanging drops of an essential gene product (2.5 μl, 10 mg/ml) in 20 mM Tris, pH=8.0, 100 mM NaCl are mixed with an equal amount of reservoir buffer containing 2.7-3.2 M sodium formate and 100 mM Tris buffer, pH=8.0, and kept at 4° C. Crystal showers may appear after 1-2 days with large single crystals growing to full size (0.3×0.3×0.15 mmd) within 2-3 weeks. Crystals are harvested in 3.5 M sodium formate and 100 mM Tris buffer, pH=8.0 and cryoprotected in 3.5 M sodium formate, 100 mM Tris buffer, pH=8.0, 10% (w/v) sucrose, and 10% (v/v) ethylene glycol before flash freezing in liquid propane.

In some embodiments, the crystal may be obtained using the methods described in U.S. Pat. No. 5,869,604, the disclosure of which is incorporated herein by reference in its entirety. The method involves (a) contacting a mixture containing uncrystallized polypeptides with an exogenous nucleating agent that has an areal lattice match of at least 90.4% to the polypeptide,(b) crystallizing the polypeptides, thereby forming at least one crystal of the polypeptide attached to the nucleating agent, the attached crystal being of a high purity, and at least one polypeptide crystal unattached to the nucleating agent, the unattached crystal being of a lower purity than the attached crystal, and (c) separating the crystal attached to the nucleating agent from the crystal unattached to the nucleating agent. The crystallized polypeptide may also be purified from contaminants by (a) contacting a mixture containing uncrystallized polypeptides and a contaminant with an exogenous nucleating agent that has an areal lattice match of at least 90.4% to the polypeptide, (b) crystallizing the polypeptides, thereby forming at least one crystal of the polypeptide attached to the nucleating agent, the attached crystal being of a high purity and produced in a high yield, and at least one crystal unattached to the nucleating agent, the unattached crystal being of a lower purity than the attached crystal, and (c) separating the crystal attached to the nucleating agent from the crystal unattached to the nucleating agent.

Once a crystal of the present invention is grown, X-ray diffraction data can be collected using methods familiar to those skilled in the art. Therefore, any person with skill in the art of protein crystallization having the present teachings and without undue experimentation can crystallize a large number of alternative forms of the essential gene products from a variety of different organisms, or polypeptides having conservative substitutions in their amino acid sequence.

A crystal lattice is defined by the symmetry of its unit cell and any structural motifs the unit cell contains. For example, there are 230 possible symmetry groups for an arbitrary crystal lattice, while the unit cell of the crystal lattice group may have an arbitrary dimension that depends on the molecules making up the lattice. Biological macromolecules, however, have asymmetric centers and are limited to 65 of the 230 symmetry groups. See Cantor et al., Biophysical Chemistry, Vol. III, W. H. Freeman & Company (1980), the disclosure of which is incorporated herein by reference in its entirety.

A crystal lattice interacts with electromagnetic or particle waves, such as X-rays or electron beams respectively, that have a wavelength with the same order of magnitude as the spacing between atoms in the unit cell. The diffracted waves are measured as an array of spots on a detection surface positioned adjacent to the crystal. Each spot has a three-dimensional position, hkl, and an intensity, I(hkl), both of which are used to reconstruct the three-dimensional electron density of the crystal with the so-called Electron Density Equation. The Electron Density Equation states that the three-dimensional electron density of the unit cell is the Fourier transform of the structure factors. Thus, in theory, if the structure factors are known for a sufficient number of spots in the detection space, then the three-dimensional electron density of the unit cell could be calculated using the Electron Density Equation.

In some embodiments of the present invention, an image of a crystal of a gene product required for proliferation or a portion thereof is obtained with the aid of a digital computer and the crystal's diffraction pattern as described in U.S. Pat. No. 5,353,236, the disclosure of which is incorporated herein by reference in its entirety. The diffraction pattern contains a plurality of reflections, each having an associated resolution. The image is obtained by (a) converting the diffraction pattern of the crystal into computer usable normalized amplitudes, the pattern being produced with a diffractometer; (b) determining from the diffraction pattern a dimension of a unit cell of the crystal; (c) providing an envelope defining the region of the unit cell occupied by the gene product or portion thereof in the crystal; (d) distributing a collection of scattering bodies within said envelope, the collection of scattering bodies having various arrangements, each of which has an associated pattern of Fourier amplitudes; (e) condensing the collection of scattering bodies to a condensed arrangement that results in a high correlation between a diffraction pattern and the pattern of Fourier amplitudes for said collection of scattering bodies; (f) determining the phase associated with at least one of the reflections of said diffraction pattern from the condensed arrangement of scattering bodies; (g) calculating an electron density distribution of the gene product or portion thereof within the unit cell from the phase determined in procedure f; and (h) displaying a graphical image of the gene product or portion thereof constructed from said electron density distribution.

The crystals of the gene products required for proliferation may be used in drug screening methods such as those described in U.S. Pat. No. 6,156,526, the disclosure of which is incorporated herein by reference in its entirety. Briefly, in such methods, a compound which inhibits the formation of a complex comprising the gene product or a portion thereof is identified as follows. A set of atomic coordinates defining the three-dimensional structure of a complex including the gene product of interest or a portion thereof are determined. A potential compound that binds to the gene product or a portion thereof involved in complex formation is selected using the atomic coordinates obtained above. The compound is contacted with the gene product or portion thereof and its binding partner(s) in the complex under conditions which would permit the complex to form in the absence of the potential compound. The binding affinity of the gene product or portion thereof for its binding partner(s) is determined and a potential compound is identified as a compound that inhibits the formation of the complex when there is a decrease in the binding affinity of the gene product or portion thereof for its binding partner(s).

In some embodiments of the present invention, the three dimensional structure of the essential gene product is determined and potential agonists and/or potential antagonists are designed with the aid of computer modeling [Bugg et al., Scientific American, Dec.:92-98 (1993); West et al., TIPS, 16:67-74 (1995); Dunbrack et al., Folding & Design, 2:27-42 (1997), the disclosures of which are incorporated herein by reference in their entireties].

Computer analysis may be performed with one or more of the computer programs including: QUANTA, CHARMM, INSIGHT, SYBYL, MACROMODEL and ICM [Dunbrack et al., Folding & Design, 2:27-42 (1997), the disclosure of which is incorporated herein by reference in its entirety]. In a further embodiment of this aspect of the invention, an initial drug-screening assay is performed using the three-dimensional structure so obtained, preferably along with a docking computer program. Such computer modeling can be performed with one or more Docking programs such as FlexX, DOC, GRAM and AUTO DOCK [Dunbrack et al., Folding & Design, 2:27-42 (1997)].

It should be understood that for each drug screening assay provided herein, a number of iterative cycles of any or all of the steps may be performed to optimize the selection. The drug screening assays of the present invention may use any of a number of means for determining the interaction between an agent or drug and an essential gene product.

In some embodiments of the present invention, a drug can be specifically designed to bind to an essential gene product of the present invention through NMR based methodology. [Shuker et al., pi Science 274:1531-1534 (1996) the disclosure of which is incorporated herein by reference herein in its entirety.] NMR spectra may be recorded using devices familiar to those skilled in the art, such as the Varian Unity Plus 500 and unity 600 spectrometers, each equipped with a pulsed-field gradient triple resonance probe as analyzed as described in Bagby et al., [Cell 82:857-867 (1995), the disclosure of which is incorporated herein by reference in its entirety]. Sequential resonance assignments of backbone ¹H, .¹⁵ N, and .¹³C atoms may be made using a combination of triple resonance experiments similar to those previously described [Bagby et al., Biochemistry, 33:2409-2421 (1994a), the disclosure of which is incorporated herein by reference in its entirety], except with enhanced sensitivity [Muhandiram and Kay, J. Magn. Reson., 103: 203-216 (1994), the disclosure of which is incorporated herein by reference in its entirety] and minimal H₂O saturation [Kay et al., J. Magn. Reson., 109:129-133 (1994), the disclosure of which is incorporated herein by reference in its entirety]. Side chain ¹H and ¹³ C assignments may be made using HCCH-TOCSY [Bax et al., J. Magn. Reson., 87:620-627 (1990), the disclosure of which is incorporated herein by reference in its entirety] experiments with mixing times of 8 ms and 16 ms.in solution but need not be included in structure calculations. Nuclear Overhauser effect (NOE) cross peaks in two-dimensional ¹H-¹H NOE spectroscopy (NOESY), three-dimensional ¹⁵N-edited NOESY-HSQC [Zhang et al., J. Biomol, NMR, 4:845-858 (1994), the disclosure of which is incorporated herein by reference in its entirety] and three-dimensional simultaneous acquisition ¹⁵N/¹³C-edited NOE [Pascal et al., J. Magn. Reson., 103:197-201 (1994), the disclosure of which is incorporated herein by reference in its entirety] spectra may be obtained with 100 ms NOE mixing times. Standard pseudo-atom distance corrections [Wuthrich et al., J. Mol. Biol., 169:949-961 (1983), the disclosure of which is incorporated herein by reference in its entirety] may be incorporated to account for center averaging. An additional 0.5 .ANG. may be added to the upper limits for distances involving methyl groups [Wagner et al., J. Mol. Biol., 196:611-639 (1987); Clore et al., Biochemistry, 26:8012-8023 (1987), the disclosures of which are incorporated herein by reference in their entireties].

The structures can be calculated using a simulated annealing protocol [Nilges et al., In computational Aspects of the Study of Biological Macromolecules by Nuclear Magnetic Resonance Spectroscopy, J. C. Hoch, F. M. Poulsen, and C. Redfield, eds., New York: Plenum Press, pp. 451-455 (1991, the disclosures of which are incorporated herein by reference in their entireties] within X-PLOR [Brunger, X-PLOR Manual, Version 3.1, New Haven, Conn.: Department of Molecular Biophysics and Biochemistry, Yale University (1993), the disclosure of which is incorporated herein by reference in its entirety] using the previously described strategy [Bagby et al., Structure, 2:107-122 (1994b), the disclosure of which is incorporated herein by reference in its entirety]. Interhelical anges may be calculated using a program written by K. Yap. Accessible surface areas were calculated using the program Naccess, available from Prof. J. Thornton, University College, London.

Compounds capable of reducing the activity or amount of gene products required for cellular proliferation may be identified using the methods described in U.S. Pat. No. 6,077,682, the disclosure of which is incorporated herein by reference in its entirety. Briefly, the three-dimensional structure of the gene product or portion thereof may be used in a drug screening assay by (a) selecting a potential drug by performing rational drug design with the three-dimensional structure determined from one or more sets of atomic coordinates of the gene product or portion thereof in conjunction with computer modeling; (b) contacting the potential drug with a polypeptide comprising the gene product or portion thereof and (c) detecting the binding of the potential drug with said polypeptide; wherein a potential drug is selected as a drug if the potential drug binds to the polypeptide. In some methods, the three-dimensional structure of the gene product or portion thereof is used in a drug screening assay involving (a) selecting a potential drug by performing structural based rotational drug design with the three-dimensional structure of the gene product or portion thereof; wherein said selecting is performed in conjunction with computer modeling; (b) contacting the potential drug with a polypeptide comprising the gene product or portion thereof in the presence of a substrate of the gene product; wherein in the absence of the potential drug the substrate is acted upon by the gene product; and (c) determining the extent to which the gene product acted upon the substrate; wherein a drug is selected when a decrease in the action of the gene product on the substrate is determined in the presence of the potential drug relative to in its absence. In some embodiments, the preceding method further involves(d) contacting the potential drug with the gene product or portion thereof for NMR analysis; wherein a binding complex forms between the potential drug and said gene product or portion thereof for NMR analysis; wherein the gene product or portion thereof for NMR analysis comprises a conservative amino acid substitution; (e) determining the three-dimensional structure of the binding complex by NMR; and (f) selecting a candidate drug by performing structural based rational drug design with the three-dimensional structure determined for the binding complex; wherein said selecting is performed in conjunction with computer modeling; (g) contacting the candidate drug with a second polypeptide comprising the gene product or portion thereof in the presence of a substrate of the gene product or portion thereof; wherein in the absence of the candidate drug the substrate is acted upon by the second polypeptide; and (h) determining the amount of action of the second polypeptide on the substrate; wherein a drug is selected when a decrease in the amount of action of the second polypeptide is determined in the presence of the candidate drug relative to in its absence.

Once the three-dimensional structure of a crystal comprising an essential gene product is determined, a potential modulator of its activity, can be examined through the use of computer modeling using a docking program such as FlexX, GRAM, DOCK, or AUTODOCK [Dunbrack et al., 1997, supra], to identify potential modulators. This procedure can include computer fitting of potential modulators to the polypeptide or fragments thereof to ascertain how well the shape and the chemical structure of the potential modulator will bind. Computer programs can also be employed to estimate the attraction, repulsion, and steric hindrance of the two binding partners (e.g., the essential gene product and a potential modulator). Generally the tighter the fit, the lower the steric hindrances, and the greater the attractive forces, the more potent the potential modulator since these properties are consistent with a tighter binding constant. Furthermore, the more specificity in the design of a potential drug the more likely that the drug will not interact as well with other proteins. This will minimize potential side-effects due to unwanted interactions with other proteins.

Compound and compound analogs can be systematically modified by computer modeling programs until one or more promising potential analogs is identified. In addition systematic modification of selected analogs can then be systematically modified by computer modeling programs until one or more potential analogs are identified. Such analysis has been shown to be effective in the development of HIV protease inhibitors [Lam et al., Science 263:380-384 (1994); Wlodawer et al., Ann. Rev. Biochem. 62:543-585 (1993); Appelt, Perspectives in Drug Discovery and Design 1:23-48 (1993); Erickson, Perspectives in Drug Discovery and Design 1:109-128 (1993), the disclosures of which are incorporated herein by reference in their entireties]. Alternatively a potential modulator could be obtained by initially screening a random peptide library produced by recombinant bacteriophage for example, [Scott and Smith, Science, 249:386-390 (1990); Cwirla et al., Proc. Natl. Acad. Sci., 87:6378-6382 (1990); Devlin et al., Science, 249:404-406 (1990), the disclosures of which are incorporated herein by reference in their entireties]. A peptide selected in this manner would then be systematically modified by computer modeling programs as described above, and then treated analogously to a structural analog.

Example 45 describes computer modelling of the structures of gene products required for proliferation.

### Example 50

### Determination of the Structure of Gene Products Required for Proliferation Using Computer Modelling

Three dimensional models were built by applying computer modelling methods to some of the gene products required for proliferation of Staphylococcus aureus using the amino acid sequences of the encoded proteins as follows. Sir Tom Blundell's program COMPOSER as provided by Tripos Associates in their BIOPOLYMER module to SYBYL was used to build the models. Skolnik's method of topology fingerprinting as implemented in Matchmaker was used to score the average mutation free energy. This number is in Boltzmans (units of kT) and should be negative (the more negative, the better the model.

Composer uses a Needleman Wunsch alignment with jumbling to find significant alignments. The reported parameters are percent identity and significance as measured from the jumbling. Those matches which were 30% identical and had a significance greater that 4 on the scale were judged to be good candidates for model building templates. If no three dimensional structures met these criteria, then a BLAST search was conducted against the most recent PDB sequence database. Any significant hits discovered in this manner were then added to the binary protein structure database and the candidate search was repeated in the manner discussed above.

In the next phase, Composer assigned structurally conserved and structurally variable regions and built the backbone structure and then searched the database for structures of the variable loops. These were then spliced in and a model of the protein resulted. Any loops (variable regions) which were unassignable were manually built and refined with a combination of dynamics.

The structure was then refined. Hydrogen atoms were added and a non-active aggregate was defined. 1000 pS of dynamics using AMBER ALL-ATOM and Kollman charges are performed. Next a minimization cycle of up 5000 steepest decent steps were performed and then the aggregate was thawed and the process was repeated on the entire protein.

The resulting structure was then validated in MATCHMAKER. The topologicaly scanned free energy determined from empirically derived protein topologies was computed and the average energy/residue is reported in Boltzamans was reported. As this number represents a free energy the more negative it is the more favorable it is.

Sixty six proteins required for the proliferation of Staphylococcus aureus were modelled as described above. MATCHMAKER energies were computed for these.

The distribution of the models built by class is shown in the table below.

TABLE 1
Distribution of models built with their MATCHMAKER energies in kT

| | | Average Matchmaker |
|---|---|---|
| Classification | Number of Models | Energy |
| Acylases | 1 | −0.10 |
| Dehydrogenases | 3 | −0.12 |
| DNA Related | 3 | −0.12 |
| Heat Shock Protein | 2 | −0.16 |
| Hydrolases | 3 | −0.16 |
| Isomerases | 1 | −0.05 |
| Ligases | 7 | −0.07 |
| Lyases | 1 | −0.09 |
| Membrane Anchored | 1 | −0.12 |
| Misc | 18 | −0.21 |
| Oxidoreductases | 6 | −0.09 |
| Proteases | 1 | −0.03 |
| Ribosome | 3 | −0.11 |
| Synthases | 4 | −0.14 |
| Transferases | 6 | −0.12 |

The validity of the above method was confirmed using FtsZ. In the case of FtsZ, a crystal structure from M. Janeschi was available. Examination of the gross structural features determined using the above modelling showed all of the folds in the 20 correct place, although there were some minor differences from the structure determined by x-ray crystallography.

### Example 51

### Functional Complementation

In another embodiment, gene products whose activities may be complemented by a proliferation-required gene product from Staphylococcus aureus, Salmonella typhimurium, Klebsiella pneumoniae, Pseudomonas aeruginosa, Enterococcus faecalis, Escherichia coli, Enterococcus faecalis, Haemophilus influenzae, Helicobacter pylori, or Salmonella typhi or homologous polypeptides are identified using merodiploids, created by introducing a plasmid or Bacterial Artificial Chromosome into an organism having a mutation in the essential gene which reduces or eliminates the activity of the gene product. In some embodiments, the mutation may be a conditional mutation, such as a temperature sensitive mutation, such that the organism proliferates under permissive conditions but is unable to proliferate under non-permissive conditions in the absence of complementation by the gene on the plasmid or Bacterial Artificial Chromosome. Alternatively, duplications may be constructed as described in Roth et al. (1987) Biosynthesis of Aromatic Amino Acids in Escherichia coli and Salmonella typhimurium, F. C. Neidhardt, ed., American Society for Microbiology, publisher, pp. 2269-2270, the disclosure of which is incorporated herein by reference in its entirety. Such methods are familiar to those skilled in the art.

Table VIII provides a cross reference for SEQ ID NOs. of the nucleotide sequences discussed herein and the SEQ ID NOs. of the polypeptides encoded by these nucleotide.

All documents cited herein are incorporated herein by reference in their entireties.

TABLE VIII

| Nucleotide SeqID | Protein SeqID | Nucleotide SeqID | Protein SeqID |
|---|---|---|---|
| 5916 | 10013 | 5961 | 10058 |
| 5917 | 10014 | 5962 | 10059 |
| 5918 | 10015 | 5963 | 10060 |
| 5919 | 10016 | 5964 | 10061 |
| 5920 | 10017 | 5965 | 10062 |
| 5921 | 10018 | 5966 | 10063 |
| 5922 | 10019 | 5967 | 10064 |
| 5923 | 10020 | 5968 | 10065 |
| 5924 | 10021 | 5969 | 10066 |
| 5925 | 10022 | 5970 | 10067 |
| 5926 | 10023 | 5971 | 10068 |
| 5927 | 10024 | 5972 | 10069 |
| 5928 | 10025 | 5973 | 10070 |
| 5929 | 10026 | 5974 | 10071 |
| 5930 | 10027 | 5975 | 10072 |
| 5931 | 10028 | 5976 | 10073 |
| 5932 | 10029 | 5977 | 10074 |
| 5933 | 10030 | 5978 | 10075 |
| 5934 | 10031 | 5979 | 10076 |
| 5935 | 10032 | 5980 | 10077 |
| 5936 | 10033 | 5981 | 10078 |
| 5937 | 10034 | 5982 | 10079 |
| 5938 | 10035 | 5983 | 10080 |
| 5939 | 10036 | 5984 | 10081 |
| 5940 | 10037 | 5985 | 10082 |
| 5941 | 10038 | 5986 | 10083 |
| 5942 | 10039 | 5987 | 10084 |
| 5943 | 10040 | 5988 | 10085 |
| 5944 | 10041 | 5989 | 10086 |
| 5945 | 10042 | 5990 | 10087 |
| 5946 | 10043 | 5991 | 10088 |
| 5947 | 10044 | 5992 | 10089 |
| 5948 | 10045 | 5993 | 10090 |
| 5949 | 10046 | 5994 | 10091 |
| 5950 | 10047 | 5995 | 10092 |
| 5951 | 10048 | 5996 | 10093 |
| 5952 | 10049 | 5997 | 10094 |
| 5953 | 10050 | 5998 | 10095 |
| 5954 | 10051 | 5999 | 10096 |
| 5955 | 10052 | 6000 | 10097 |
| 5956 | 10053 | 6001 | 10098 |
| 5957 | 10054 | 6002 | 10099 |
| 5958 | 10055 | 6003 | 10100 |
| 5959 | 10056 | 6004 | 10101 |
| 5960 | 10057 | 6005 | 10102 |
| 6006 | 10103 | 6053 | 10150 |
| 6007 | 10104 | 6054 | 10151 |
| 6008 | 10105 | 6055 | 10152 |
| 6009 | 10106 | 6056 | 10153 |
| 6010 | 10107 | 6057 | 10154 |
| 6011 | 10108 | 6058 | 10155 |
| 6012 | 10109 | 6059 | 10156 |
| 6013 | 10110 | 6060 | 10157 |
| 6014 | 10111 | 6061 | 10158 |
| 6015 | 10112 | 6062 | 10159 |
| 6016 | 10113 | 6063 | 10160 |
| 6017 | 10114 | 6064 | 10161 |
| 6018 | 10115 | 6065 | 10162 |
| 6019 | 10116 | 6066 | 10163 |
| 6020 | 10117 | 6067 | 10164 |
| 6021 | 10118 | 6068 | 10165 |
| 6022 | 10119 | 6069 | 10166 |
| 6023 | 10120 | 6070 | 10167 |
| 6024 | 10121 | 6071 | 10168 |
| 6025 | 10122 | 6072 | 10169 |
| 6026 | 10123 | 6073 | 10170 |
| 6027 | 10124 | 6074 | 10171 |
| 6028 | 10125 | 6075 | 10172 |
| 6029 | 10126 | 6076 | 10173 |
| 6030 | 10127 | 6077 | 10174 |
| 6031 | 10128 | 6078 | 10175 |
| 6032 | 10129 | 6079 | 10176 |
| 6033 | 10130 | 6080 | 10177 |
| 6034 | 10131 | 6081 | 10178 |
| 6035 | 10132 | 6082 | 10179 |
| 6036 | 10133 | 6083 | 10180 |
| 6037 | 10134 | 6084 | 10181 |
| 6038 | 10135 | 6085 | 10182 |
| 6039 | 10136 | 6086 | 10183 |
| 6040 | 10137 | 6087 | 10184 |
| 6041 | 10138 | 6088 | 10185 |
| 6042 | 10139 | 6089 | 10186 |
| 6043 | 10140 | 6090 | 10187 |
| 6044 | 10141 | 6091 | 10188 |
| 6045 | 10142 | 6092 | 10189 |
| 6046 | 10143 | 6093 | 10190 |
| 6047 | 10144 | 6094 | 10191 |
| 6048 | 10145 | 6095 | 10192 |
| 6049 | 10146 | 6096 | 10193 |
| 6050 | 10147 | 6097 | 10194 |
| 6051 | 10148 | 6098 | 10195 |
| 6052 | 10149 | 6099 | 10196 |
| 6100 | 10197 | 6147 | 10244 |
| 6101 | 10198 | 6148 | 10245 |
| 6102 | 10199 | 6149 | 10246 |
| 6103 | 10200 | 6150 | 10247 |
| 6104 | 10201 | 6151 | 10248 |
| 6105 | 10202 | 6152 | 10249 |
| 6106 | 10203 | 6153 | 10250 |
| 6107 | 10204 | 6154 | 10251 |
| 6108 | 10205 | 6155 | 10252 |
| 6109 | 10206 | 6156 | 10253 |
| 6110 | 10207 | 6157 | 10254 |
| 6111 | 10208 | 6158 | 10255 |
| 6112 | 10209 | 6159 | 10256 |
| 6113 | 10210 | 6160 | 10257 |
| 6114 | 10211 | 6161 | 10258 |
| 6115 | 10212 | 6162 | 10259 |
| 6116 | 10213 | 6163 | 10260 |
| 6117 | 10214 | 6164 | 10261 |
| 6118 | 10215 | 6165 | 10262 |
| 6119 | 10216 | 6166 | 10263 |
| 6120 | 10217 | 6167 | 10264 |
| 6121 | 10218 | 6168 | 10265 |
| 6122 | 10219 | 6169 | 10266 |
| 6123 | 10220 | 6170 | 10267 |
| 6124 | 10221 | 6171 | 10268 |
| 6125 | 10222 | 6172 | 10269 |
| 6126 | 10223 | 6173 | 10270 |
| 6127 | 10224 | 6174 | 10271 |
| 6128 | 10225 | 6175 | 10272 |
| 6129 | 10226 | 6176 | 10273 |
| 6130 | 10227 | 6177 | 10274 |
| 6131 | 10228 | 6178 | 10275 |
| 6132 | 10229 | 6179 | 10276 |
| 6133 | 10230 | 6180 | 10277 |
| 6134 | 10231 | 6181 | 10278 |
| 6135 | 10232 | 6182 | 10279 |
| 6136 | 10233 | 6183 | 10280 |
| 6137 | 10234 | 6184 | 10281 |
| 6138 | 10235 | 6185 | 10282 |
| 6139 | 10236 | 6186 | 10283 |
| 6140 | 10237 | 6187 | 10284 |
| 6141 | 10238 | 6188 | 10285 |
| 6142 | 10239 | 6189 | 10286 |
| 6143 | 10240 | 6190 | 10287 |
| 6144 | 10241 | 6191 | 10288 |
| 6145 | 10242 | 6192 | 10289 |
| 6146 | 10243 | 6193 | 10290 |
| 6194 | 10291 | 6241 | 10338 |
| 6195 | 10292 | 6242 | 10339 |
| 6196 | 10293 | 6243 | 10340 |
| 6197 | 10294 | 6244 | 10341 |
| 6198 | 10295 | 6245 | 10342 |
| 6199 | 10296 | 6246 | 10343 |
| 6200 | 10297 | 6247 | 10344 |
| 6201 | 10298 | 6248 | 10345 |
| 6202 | 10299 | 6249 | 10346 |
| 6203 | 10300 | 6250 | 10347 |
| 6204 | 10301 | 6251 | 10348 |
| 6205 | 10302 | 6252 | 10349 |
| 6206 | 10303 | 6253 | 10350 |
| 6207 | 10304 | 6254 | 10351 |
| 6208 | 10305 | 6255 | 10352 |
| 6209 | 10306 | 6256 | 10353 |
| 6210 | 10307 | 6257 | 10354 |
| 6211 | 10308 | 6258 | 10355 |
| 6212 | 10309 | 6259 | 10356 |
| 6213 | 10310 | 6260 | 10357 |
| 6214 | 10311 | 6261 | 10358 |
| 6215 | 10312 | 6262 | 10359 |
| 6216 | 10313 | 6263 | 10360 |
| 6217 | 10314 | 6264 | 10361 |
| 6218 | 10315 | 6265 | 10362 |
| 6219 | 10316 | 6266 | 10363 |
| 6220 | 10317 | 6267 | 10364 |
| 6221 | 10318 | 6268 | 10365 |
| 6222 | 10319 | 6269 | 10366 |
| 6223 | 10320 | 6270 | 10367 |
| 6224 | 10321 | 6271 | 10368 |
| 6225 | 10322 | 6272 | 10369 |
| 6226 | 10323 | 6273 | 10370 |
| 6227 | 10324 | 6274 | 10371 |
| 6228 | 10325 | 6275 | 10372 |
| 6229 | 10326 | 6276 | 10373 |
| 6230 | 10327 | 6277 | 10374 |
| 6231 | 10328 | 6278 | 10375 |
| 6232 | 10329 | 6279 | 10376 |
| 6233 | 10330 | 6280 | 10377 |
| 6234 | 10331 | 6281 | 10378 |
| 6235 | 10332 | 6282 | 10379 |
| 6236 | 10333 | 6283 | 10380 |
| 6237 | 10334 | 6284 | 10381 |
| 6238 | 10335 | 6285 | 10382 |
| 6239 | 10336 | 6286 | 10383 |
| 6240 | 10337 | 6287 | 10384 |
| 6288 | 10385 | 6335 | 10432 |
| 6289 | 10386 | 6336 | 10433 |
| 6290 | 10387 | 6337 | 10434 |
| 6291 | 10388 | 6338 | 10435 |
| 6292 | 10389 | 6339 | 10436 |
| 6293 | 10390 | 6340 | 10437 |
| 6294 | 10391 | 6341 | 10438 |
| 6295 | 10392 | 6342 | 10439 |
| 6296 | 10393 | 6343 | 10440 |
| 6297 | 10394 | 6344 | 10441 |
| 6298 | 10395 | 6345 | 10442 |
| 6299 | 10396 | 6346 | 10443 |
| 6300 | 10397 | 6347 | 10444 |
| 6301 | 10398 | 6348 | 10445 |
| 6302 | 10399 | 6349 | 10446 |
| 6303 | 10400 | 6350 | 10447 |
| 6304 | 10401 | 6351 | 10448 |
| 6305 | 10402 | 6352 | 10449 |
| 6306 | 10403 | 6353 | 10450 |
| 6307 | 10404 | 6354 | 10451 |
| 6308 | 10405 | 6355 | 10452 |
| 6309 | 10406 | 6356 | 10453 |
| 6310 | 10407 | 6357 | 10454 |
| 6311 | 10408 | 6358 | 10455 |
| 6312 | 10409 | 6359 | 10456 |
| 6313 | 10410 | 6360 | 10457 |
| 6314 | 10411 | 6361 | 10458 |
| 6315 | 10412 | 6362 | 10459 |
| 6316 | 10413 | 6363 | 10460 |
| 6317 | 10414 | 6364 | 10461 |
| 6318 | 10415 | 6365 | 10462 |
| 6319 | 10416 | 6366 | 10463 |
| 6320 | 10417 | 6367 | 10464 |
| 6321 | 10418 | 6368 | 10465 |
| 6322 | 10419 | 6369 | 10466 |
| 6323 | 10420 | 6370 | 10467 |
| 6324 | 10421 | 6371 | 10468 |
| 6325 | 10422 | 6372 | 10469 |
| 6326 | 10423 | 6373 | 10470 |
| 6327 | 10424 | 6374 | 10471 |
| 6328 | 10425 | 6375 | 10472 |
| 6329 | 10426 | 6376 | 10473 |
| 6330 | 10427 | 6377 | 10474 |
| 6331 | 10428 | 6378 | 10475 |
| 6332 | 10429 | 6379 | 10476 |
| 6333 | 10430 | 6380 | 10477 |
| 6334 | 10431 | 6381 | 10478 |
| 6382 | 10479 | 6429 | 10526 |
| 6383 | 10480 | 6430 | 10527 |
| 6384 | 10481 | 6431 | 10528 |
| 6385 | 10482 | 6432 | 10529 |
| 6386 | 10483 | 6433 | 10530 |
| 6387 | 10484 | 6434 | 10531 |
| 6388 | 10485 | 6435 | 10532 |
| 6389 | 10486 | 6436 | 10533 |
| 6390 | 10487 | 6437 | 10534 |
| 6391 | 10488 | 6438 | 10535 |
| 6392 | 10489 | 6439 | 10536 |
| 6393 | 10490 | 6440 | 10537 |
| 6394 | 10491 | 6441 | 10538 |
| 6395 | 10492 | 6442 | 10539 |
| 6396 | 10493 | 6443 | 10540 |
| 6397 | 10494 | 6444 | 10541 |
| 6398 | 10495 | 6445 | 10542 |
| 6399 | 10496 | 6446 | 10543 |
| 6400 | 10497 | 6447 | 10544 |
| 6401 | 10498 | 6448 | 10545 |
| 6402 | 10499 | 6449 | 10546 |
| 6403 | 10500 | 6450 | 10547 |
| 6404 | 10501 | 6451 | 10548 |
| 6405 | 10502 | 6452 | 10549 |
| 6406 | 10503 | 6453 | 10550 |
| 6407 | 10504 | 6454 | 10551 |
| 6408 | 10505 | 6455 | 10552 |
| 6409 | 10506 | 6456 | 10553 |
| 6410 | 10507 | 6457 | 10554 |
| 6411 | 10508 | 6458 | 10555 |
| 6412 | 10509 | 6459 | 10556 |
| 6413 | 10510 | 6460 | 10557 |
| 6414 | 10511 | 6461 | 10558 |
| 6415 | 10512 | 6462 | 10559 |
| 6416 | 10513 | 6463 | 10560 |
| 6417 | 10514 | 6464 | 10561 |
| 6418 | 10515 | 6465 | 10562 |
| 6419 | 10516 | 6466 | 10563 |
| 6420 | 10517 | 6467 | 10564 |
| 6421 | 10518 | 6468 | 10565 |
| 6422 | 10519 | 6469 | 10566 |
| 6423 | 10520 | 6470 | 10567 |
| 6424 | 10521 | 6471 | 10568 |
| 6425 | 10522 | 6472 | 10569 |
| 6426 | 10523 | 6473 | 10570 |
| 6427 | 10524 | 6474 | 10571 |
| 6428 | 10525 | 6475 | 10572 |
| 6476 | 10573 | 6523 | 10620 |
| 6477 | 10574 | 6524 | 10621 |
| 6478 | 10575 | 6525 | 10622 |
| 6479 | 10576 | 6526 | 10623 |
| 6480 | 10577 | 6527 | 10624 |
| 6481 | 10578 | 6528 | 10625 |
| 6482 | 10579 | 6529 | 10626 |
| 6483 | 10580 | 6530 | 10627 |
| 6484 | 10581 | 6531 | 10628 |
| 6485 | 10582 | 6532 | 10629 |
| 6486 | 10583 | 6533 | 10630 |
| 6487 | 10584 | 6534 | 10631 |
| 6488 | 10585 | 6535 | 10632 |
| 6489 | 10586 | 6536 | 10633 |
| 6490 | 10587 | 6537 | 10634 |
| 6491 | 10588 | 6538 | 10635 |
| 6492 | 10589 | 6539 | 10636 |
| 6493 | 10590 | 6540 | 10637 |
| 6494 | 10591 | 6541 | 10638 |
| 6495 | 10592 | 6542 | 10639 |
| 6496 | 10593 | 6543 | 10640 |
| 6497 | 10594 | 6544 | 10641 |
| 6498 | 10595 | 6545 | 10642 |
| 6499 | 10596 | 6546 | 10643 |
| 6500 | 10597 | 6547 | 10644 |
| 6501 | 10598 | 6548 | 10645 |
| 6502 | 10599 | 6549 | 10646 |
| 6503 | 10600 | 6550 | 10647 |
| 6504 | 10601 | 6551 | 10648 |
| 6505 | 10602 | 6552 | 10649 |
| 6506 | 10603 | 6553 | 10650 |
| 6507 | 10604 | 6554 | 10651 |
| 6508 | 10605 | 6555 | 10652 |
| 6509 | 10606 | 6556 | 10653 |
| 6510 | 10607 | 6557 | 10654 |
| 6511 | 10608 | 6558 | 10655 |
| 6512 | 10609 | 6559 | 10656 |
| 6513 | 10610 | 6560 | 10657 |
| 6514 | 10611 | 6561 | 10658 |
| 6515 | 10612 | 6562 | 10659 |
| 6516 | 10613 | 6563 | 10660 |
| 6517 | 10614 | 6564 | 10661 |
| 6518 | 10615 | 6565 | 10662 |
| 6519 | 10616 | 6566 | 10663 |
| 6520 | 10617 | 6567 | 10664 |
| 6521 | 10618 | 6568 | 10665 |
| 6522 | 10619 | 6569 | 10666 |
| 6570 | 10667 | 6617 | 10714 |
| 6571 | 10668 | 6618 | 10715 |
| 6572 | 10669 | 6619 | 10716 |
| 6573 | 10670 | 6620 | 10717 |
| 6574 | 10671 | 6621 | 10718 |
| 6575 | 10672 | 6622 | 10719 |
| 6576 | 10673 | 6623 | 10720 |
| 6577 | 10674 | 6624 | 10721 |
| 6578 | 10675 | 6625 | 10722 |
| 6579 | 10676 | 6626 | 10723 |
| 6580 | 10677 | 6627 | 10724 |
| 6581 | 10678 | 6628 | 10725 |
| 6582 | 10679 | 6629 | 10726 |
| 6583 | 10680 | 6630 | 10727 |
| 6584 | 10681 | 6631 | 10728 |
| 6585 | 10682 | 6632 | 10729 |
| 6586 | 10683 | 6633 | 10730 |
| 6587 | 10684 | 6634 | 10731 |
| 6588 | 10685 | 6635 | 10732 |
| 6589 | 10686 | 6636 | 10733 |
| 6590 | 10687 | 6637 | 10734 |
| 6591 | 10688 | 6638 | 10735 |
| 6592 | 10689 | 6639 | 10736 |
| 6593 | 10690 | 6640 | 10737 |
| 6594 | 10691 | 6641 | 10738 |
| 6595 | 10692 | 6642 | 10739 |
| 6596 | 10693 | 6643 | 10740 |
| 6597 | 10694 | 6644 | 10741 |
| 6598 | 10695 | 6645 | 10742 |
| 6599 | 10696 | 6646 | 10743 |
| 6600 | 10697 | 6647 | 10744 |
| 6601 | 10698 | 6648 | 10745 |
| 6602 | 10699 | 6649 | 10746 |
| 6603 | 10700 | 6650 | 10747 |
| 6604 | 10701 | 6651 | 10748 |
| 6605 | 10702 | 6652 | 10749 |
| 6606 | 10703 | 6653 | 10750 |
| 6607 | 10704 | 6654 | 10751 |
| 6608 | 10705 | 6655 | 10752 |
| 6609 | 10706 | 6656 | 10753 |
| 6610 | 10707 | 6657 | 10754 |
| 6611 | 10708 | 6658 | 10755 |
| 6612 | 10709 | 6659 | 10756 |
| 6613 | 10710 | 6660 | 10757 |
| 6614 | 10711 | 6661 | 10758 |
| 6615 | 10712 | 6662 | 10759 |
| 6616 | 10713 | 6663 | 10760 |
| 6664 | 10761 | 6711 | 10808 |
| 6665 | 10762 | 6712 | 10809 |
| 6666 | 10763 | 6713 | 10810 |
| 6667 | 10764 | 6714 | 10811 |
| 6668 | 10765 | 6715 | 10812 |
| 6669 | 10766 | 6716 | 10813 |
| 6670 | 10767 | 6717 | 10814 |
| 6671 | 10768 | 6718 | 10815 |
| 6672 | 10769 | 6719 | 10816 |
| 6673 | 10770 | 6720 | 10817 |
| 6674 | 10771 | 6721 | 10818 |
| 6675 | 10772 | 6722 | 10819 |
| 6676 | 10773 | 6723 | 10820 |
| 6677 | 10774 | 6724 | 10821 |
| 6678 | 10775 | 6725 | 10822 |
| 6679 | 10776 | 6726 | 10823 |
| 6680 | 10777 | 6727 | 10824 |
| 6681 | 10778 | 6728 | 10825 |
| 6682 | 10779 | 6729 | 10826 |
| 6683 | 10780 | 6730 | 10827 |
| 6684 | 10781 | 6731 | 10828 |
| 6685 | 10782 | 6732 | 10829 |
| 6686 | 10783 | 6733 | 10830 |
| 6687 | 10784 | 6734 | 10831 |
| 6688 | 10785 | 6735 | 10832 |
| 6689 | 10786 | 6736 | 10833 |
| 6690 | 10787 | 6737 | 10834 |
| 6691 | 10788 | 6738 | 10835 |
| 6692 | 10789 | 6739 | 10836 |
| 6693 | 10790 | 6740 | 10837 |
| 6694 | 10791 | 6741 | 10838 |
| 6695 | 10792 | 6742 | 10839 |
| 6696 | 10793 | 6743 | 10840 |
| 6697 | 10794 | 6744 | 10841 |
| 6698 | 10795 | 6745 | 10842 |
| 6699 | 10796 | 6746 | 10843 |
| 6700 | 10797 | 6747 | 10844 |
| 6701 | 10798 | 6748 | 10845 |
| 6702 | 10799 | 6749 | 10846 |
| 6703 | 10800 | 6750 | 10847 |
| 6704 | 10801 | 6751 | 10848 |
| 6705 | 10802 | 6752 | 10849 |
| 6706 | 10803 | 6753 | 10850 |
| 6707 | 10804 | 6754 | 10851 |
| 6708 | 10805 | 6755 | 10852 |
| 6709 | 10806 | 6756 | 10853 |
| 6710 | 10807 | 6757 | 10854 |
| 6758 | 10855 | 6805 | 10902 |
| 6759 | 10856 | 6806 | 10903 |
| 6760 | 10857 | 6807 | 10904 |
| 6761 | 10858 | 6808 | 10905 |
| 6762 | 10859 | 6809 | 10906 |
| 6763 | 10860 | 6810 | 10907 |
| 6764 | 10861 | 6811 | 10908 |
| 6765 | 10862 | 6812 | 10909 |
| 6766 | 10863 | 6813 | 10910 |
| 6767 | 10864 | 6814 | 10911 |
| 6768 | 10865 | 6815 | 10912 |
| 6769 | 10866 | 6816 | 10913 |
| 6770 | 10867 | 6817 | 10914 |
| 6771 | 10868 | 6818 | 10915 |
| 6772 | 10869 | 6819 | 10916 |
| 6773 | 10870 | 6820 | 10917 |
| 6774 | 10871 | 6821 | 10918 |
| 6775 | 10872 | 6822 | 10919 |
| 6776 | 10873 | 6823 | 10920 |
| 6777 | 10874 | 6824 | 10921 |
| 6778 | 10875 | 6825 | 10922 |
| 6779 | 10876 | 6826 | 10923 |
| 6780 | 10877 | 6827 | 10924 |
| 6781 | 10878 | 6828 | 10925 |
| 6782 | 10879 | 6829 | 10926 |
| 6783 | 10880 | 6830 | 10927 |
| 6784 | 10881 | 6831 | 10928 |
| 6785 | 10882 | 6832 | 10929 |
| 6786 | 10883 | 6833 | 10930 |
| 6787 | 10884 | 6834 | 10931 |
| 6788 | 10885 | 6835 | 10932 |
| 6789 | 10886 | 6836 | 10933 |
| 6790 | 10887 | 6837 | 10934 |
| 6791 | 10888 | 6838 | 10935 |
| 6792 | 10889 | 6839 | 10936 |
| 6793 | 10890 | 6840 | 10937 |
| 6794 | 10891 | 6841 | 10938 |
| 6795 | 10892 | 6842 | 10939 |
| 6796 | 10893 | 6843 | 10940 |
| 6797 | 10894 | 6844 | 10941 |
| 6798 | 10895 | 6845 | 10942 |
| 6799 | 10896 | 6846 | 10943 |
| 6800 | 10897 | 6847 | 10944 |
| 6801 | 10898 | 6848 | 10945 |
| 6802 | 10899 | 6849 | 10946 |
| 6803 | 10900 | 6850 | 10947 |
| 6804 | 10901 | 6851 | 10948 |
| 6852 | 10949 | 6899 | 10996 |
| 6853 | 10950 | 6900 | 10997 |
| 6854 | 10951 | 6901 | 10998 |
| 6855 | 10952 | 6902 | 10999 |
| 6856 | 10953 | 6903 | 11000 |
| 6857 | 10954 | 6904 | 11001 |
| 6858 | 10955 | 6905 | 11002 |
| 6859 | 10956 | 6906 | 11003 |
| 6860 | 10957 | 6907 | 11004 |
| 6861 | 10958 | 6908 | 11005 |
| 6862 | 10959 | 6909 | 11006 |
| 6863 | 10960 | 6910 | 11007 |
| 6864 | 10961 | 6911 | 11008 |
| 6865 | 10962 | 6912 | 11009 |
| 6866 | 10963 | 6913 | 11010 |
| 6867 | 10964 | 6914 | 11011 |
| 6868 | 10965 | 6915 | 11012 |
| 6869 | 10966 | 6916 | 11013 |
| 6870 | 10967 | 6917 | 11014 |
| 6871 | 10968 | 6918 | 11015 |
| 6872 | 10969 | 6919 | 11016 |
| 6873 | 10970 | 6920 | 11017 |
| 6874 | 10971 | 6921 | 11018 |
| 6875 | 10972 | 6922 | 11019 |
| 6876 | 10973 | 6923 | 11020 |
| 6877 | 10974 | 6924 | 11021 |
| 6878 | 10975 | 6925 | 11022 |
| 6879 | 10976 | 6926 | 11023 |
| 6880 | 10977 | 6927 | 11024 |
| 6881 | 10978 | 6928 | 11025 |
| 6882 | 10979 | 6929 | 11026 |
| 6883 | 10980 | 6930 | 11027 |
| 6884 | 10981 | 6931 | 11028 |
| 6885 | 10982 | 6932 | 11029 |
| 6886 | 10983 | 6933 | 11030 |
| 6887 | 10984 | 6934 | 11031 |
| 6888 | 10985 | 6935 | 11032 |
| 6889 | 10986 | 6936 | 11033 |
| 6890 | 10987 | 6937 | 11034 |
| 6891 | 10988 | 6938 | 11035 |
| 6892 | 10989 | 6939 | 11036 |
| 6893 | 10990 | 6940 | 11037 |
| 6894 | 10991 | 6941 | 11038 |
| 6895 | 10992 | 6942 | 11039 |
| 6896 | 10993 | 6943 | 11040 |
| 6897 | 10994 | 6944 | 11041 |
| 6898 | 10995 | 6945 | 11042 |
| 6946 | 11043 | 6993 | 11090 |
| 6947 | 11044 | 6994 | 11091 |
| 6948 | 11045 | 6995 | 11092 |
| 6949 | 11046 | 6996 | 11093 |
| 6950 | 11047 | 6997 | 11094 |
| 6951 | 11048 | 6998 | 11095 |
| 6952 | 11049 | 6999 | 11096 |
| 6953 | 11050 | 7000 | 11097 |
| 6954 | 11051 | 7001 | 11098 |
| 6955 | 11052 | 7002 | 11099 |
| 6956 | 11053 | 7003 | 11100 |
| 6957 | 11054 | 7004 | 11101 |
| 6958 | 11055 | 7005 | 11102 |
| 6959 | 11056 | 7006 | 11103 |
| 6960 | 11057 | 7007 | 11104 |
| 6961 | 11058 | 7008 | 11105 |
| 6962 | 11059 | 7009 | 11106 |
| 6963 | 11060 | 7010 | 11107 |
| 6964 | 11061 | 7011 | 11108 |
| 6965 | 11062 | 7012 | 11109 |
| 6966 | 11063 | 7013 | 11110 |
| 6967 | 11064 | 7014 | 11111 |
| 6968 | 11065 | 7015 | 11112 |
| 6969 | 11066 | 7016 | 11113 |
| 6970 | 11067 | 7017 | 11114 |
| 6971 | 11068 | 7018 | 11115 |
| 6972 | 11069 | 7019 | 11116 |
| 6973 | 11070 | 7020 | 11117 |
| 6974 | 11071 | 7021 | 11118 |
| 6975 | 11072 | 7022 | 11119 |
| 6976 | 11073 | 7023 | 11120 |
| 6977 | 11074 | 7024 | 11121 |
| 6978 | 11075 | 7025 | 11122 |
| 6979 | 11076 | 7026 | 11123 |
| 6980 | 11077 | 7027 | 11124 |
| 6981 | 11078 | 7028 | 11125 |
| 6982 | 11079 | 7029 | 11126 |
| 6983 | 11080 | 7030 | 11127 |
| 6984 | 11081 | 7031 | 11128 |
| 6985 | 11082 | 7032 | 11129 |
| 6986 | 11083 | 7033 | 11130 |
| 6987 | 11084 | 7034 | 11131 |
| 6988 | 11085 | 7035 | 11132 |
| 6989 | 11086 | 7036 | 11133 |
| 6990 | 11087 | 7037 | 11134 |
| 6991 | 11088 | 7038 | 11135 |
| 6992 | 11089 | 7039 | 11136 |
| 7040 | 11137 | 7087 | 11184 |
| 7041 | 11138 | 7088 | 11185 |
| 7042 | 11139 | 7089 | 11186 |
| 7043 | 11140 | 7090 | 11187 |
| 7044 | 11141 | 7091 | 11188 |
| 7045 | 11142 | 7092 | 11189 |
| 7046 | 11143 | 7093 | 11190 |
| 7047 | 11144 | 7094 | 11191 |
| 7048 | 11145 | 7095 | 11192 |
| 7049 | 11146 | 7096 | 11193 |
| 7050 | 11147 | 7097 | 11194 |
| 7051 | 11148 | 7098 | 11195 |
| 7052 | 11149 | 7099 | 11196 |
| 7053 | 11150 | 7100 | 11197 |
| 7054 | 11151 | 7101 | 11198 |
| 7055 | 11152 | 7102 | 11199 |
| 7056 | 11153 | 7103 | 11200 |
| 7057 | 11154 | 7104 | 11201 |
| 7058 | 11155 | 7105 | 11202 |
| 7059 | 11156 | 7106 | 11203 |
| 7060 | 11157 | 7107 | 11204 |
| 7061 | 11158 | 7108 | 11205 |
| 7062 | 11159 | 7109 | 11206 |
| 7063 | 11160 | 7110 | 11207 |
| 7064 | 11161 | 7111 | 11208 |
| 7065 | 11162 | 7112 | 11209 |
| 7066 | 11163 | 7113 | 11210 |
| 7067 | 11164 | 7114 | 11211 |
| 7068 | 11165 | 7115 | 11212 |
| 7069 | 11166 | 7116 | 11213 |
| 7070 | 11167 | 7117 | 11214 |
| 7071 | 11168 | 7118 | 11215 |
| 7072 | 11169 | 7119 | 11216 |
| 7073 | 11170 | 7120 | 11217 |
| 7074 | 11171 | 7121 | 11218 |
| 7075 | 11172 | 7122 | 11219 |
| 7076 | 11173 | 7123 | 11220 |
| 7077 | 11174 | 7124 | 11221 |
| 7078 | 11175 | 7125 | 11222 |
| 7079 | 11176 | 7126 | 11223 |
| 7080 | 11177 | 7127 | 11224 |
| 7081 | 11178 | 7128 | 11225 |
| 7082 | 11179 | 7129 | 11226 |
| 7083 | 11180 | 7130 | 11227 |
| 7084 | 11181 | 7131 | 11228 |
| 7085 | 11182 | 7132 | 11229 |
| 7086 | 11183 | 7133 | 11230 |
| 7134 | 11231 | 7181 | 11278 |
| 7135 | 11232 | 7182 | 11279 |
| 7136 | 11233 | 7183 | 11280 |
| 7137 | 11234 | 7184 | 11281 |
| 7138 | 11235 | 7185 | 11282 |
| 7139 | 11236 | 7186 | 11283 |
| 7140 | 11237 | 7187 | 11284 |
| 7141 | 11238 | 7188 | 11285 |
| 7142 | 11239 | 7189 | 11286 |
| 7143 | 11240 | 7190 | 11287 |
| 7144 | 11241 | 7191 | 11288 |
| 7145 | 11242 | 7192 | 11289 |
| 7146 | 11243 | 7193 | 11290 |
| 7147 | 11244 | 7194 | 11291 |
| 7148 | 11245 | 7195 | 11292 |
| 7149 | 11246 | 7196 | 11293 |
| 7150 | 11247 | 7197 | 11294 |
| 7151 | 11248 | 7198 | 11295 |
| 7152 | 11249 | 7199 | 11296 |
| 7153 | 11250 | 7200 | 11297 |
| 7154 | 11251 | 7201 | 11298 |
| 7155 | 11252 | 7202 | 11299 |
| 7156 | 11253 | 7203 | 11300 |
| 7157 | 11254 | 7204 | 11301 |
| 7158 | 11255 | 7205 | 11302 |
| 7159 | 11256 | 7206 | 11303 |
| 7160 | 11257 | 7207 | 11304 |
| 7161 | 11258 | 7208 | 11305 |
| 7162 | 11259 | 7209 | 11306 |
| 7163 | 11260 | 7210 | 11307 |
| 7164 | 11261 | 7211 | 11308 |
| 7165 | 11262 | 7212 | 11309 |
| 7166 | 11263 | 7213 | 11310 |
| 7167 | 11264 | 7214 | 11311 |
| 7168 | 11265 | 7215 | 11312 |
| 7169 | 11266 | 7216 | 11313 |
| 7170 | 11267 | 7217 | 11314 |
| 7171 | 11268 | 7218 | 11315 |
| 7172 | 11269 | 7219 | 11316 |
| 7173 | 11270 | 7220 | 11317 |
| 7174 | 11271 | 7221 | 11318 |
| 7175 | 11272 | 7222 | 11319 |
| 7176 | 11273 | 7223 | 11320 |
| 7177 | 11274 | 7224 | 11321 |
| 7178 | 11275 | 7225 | 11322 |
| 7179 | 11276 | 7226 | 11323 |
| 7180 | 11277 | 7227 | 11324 |
| 7228 | 11325 | 7275 | 11372 |
| 7229 | 11326 | 7276 | 11373 |
| 7230 | 11327 | 7277 | 11374 |
| 7231 | 11328 | 7278 | 11375 |
| 7232 | 11329 | 7279 | 11376 |
| 7233 | 11330 | 7280 | 11377 |
| 7234 | 11331 | 7281 | 11378 |
| 7235 | 11332 | 7282 | 11379 |
| 7236 | 11333 | 7283 | 11380 |
| 7237 | 11334 | 7284 | 11381 |
| 7238 | 11335 | 7285 | 11382 |
| 7239 | 11336 | 7286 | 11383 |
| 7240 | 11337 | 7287 | 11384 |
| 7241 | 11338 | 7288 | 11385 |
| 7242 | 11339 | 7289 | 11386 |
| 7243 | 11340 | 7290 | 11387 |
| 7244 | 11341 | 7291 | 11388 |
| 7245 | 11342 | 7292 | 11389 |
| 7246 | 11343 | 7293 | 11390 |
| 7247 | 11344 | 7294 | 11391 |
| 7248 | 11345 | 7295 | 11392 |
| 7249 | 11346 | 7296 | 11393 |
| 7250 | 11347 | 7297 | 11394 |
| 7251 | 11348 | 7298 | 11395 |
| 7252 | 11349 | 7299 | 11396 |
| 7253 | 11350 | 7300 | 11397 |
| 7254 | 11351 | 7301 | 11398 |
| 7255 | 11352 | 7302 | 11399 |
| 7256 | 11353 | 7303 | 11400 |
| 7257 | 11354 | 7304 | 11401 |
| 7258 | 11355 | 7305 | 11402 |
| 7259 | 11356 | 7306 | 11403 |
| 7260 | 11357 | 7307 | 11404 |
| 7261 | 11358 | 7308 | 11405 |
| 7262 | 11359 | 7309 | 11406 |
| 7263 | 11360 | 7310 | 11407 |
| 7264 | 11361 | 7311 | 11408 |
| 7265 | 11362 | 7312 | 11409 |
| 7266 | 11363 | 7313 | 11410 |
| 7267 | 11364 | 7314 | 11411 |
| 7268 | 11365 | 7315 | 11412 |
| 7269 | 11366 | 7316 | 11413 |
| 7270 | 11367 | 7317 | 11414 |
| 7271 | 11368 | 7318 | 11415 |
| 7272 | 11369 | 7319 | 11416 |
| 7273 | 11370 | 7320 | 11417 |
| 7274 | 11371 | 7321 | 11418 |
| 7322 | 11419 | 7369 | 11466 |
| 7323 | 11420 | 7370 | 11467 |
| 7324 | 11421 | 7371 | 11468 |
| 7325 | 11422 | 7372 | 11469 |
| 7326 | 11423 | 7373 | 11470 |
| 7327 | 11424 | 7374 | 11471 |
| 7328 | 11425 | 7375 | 11472 |
| 7329 | 11426 | 7376 | 11473 |
| 7330 | 11427 | 7377 | 11474 |
| 7331 | 11428 | 7378 | 11475 |
| 7332 | 11429 | 7379 | 11476 |
| 7333 | 11430 | 7380 | 11477 |
| 7334 | 11431 | 7381 | 11478 |
| 7335 | 11432 | 7382 | 11479 |
| 7336 | 11433 | 7383 | 11480 |
| 7337 | 11434 | 7384 | 11481 |
| 7338 | 11435 | 7385 | 11482 |
| 7339 | 11436 | 7386 | 11483 |
| 7340 | 11437 | 7387 | 11484 |
| 7341 | 11438 | 7388 | 11485 |
| 7342 | 11439 | 7389 | 11486 |
| 7343 | 11440 | 7390 | 11487 |
| 7344 | 11441 | 7391 | 11488 |
| 7345 | 11442 | 7392 | 11489 |
| 7346 | 11443 | 7393 | 11490 |
| 7347 | 11444 | 7394 | 11491 |
| 7348 | 11445 | 7395 | 11492 |
| 7349 | 11446 | 7396 | 11493 |
| 7350 | 11447 | 7397 | 11494 |
| 7351 | 11448 | 7398 | 11495 |
| 7352 | 11449 | 7399 | 11496 |
| 7353 | 11450 | 7400 | 11497 |
| 7354 | 11451 | 7401 | 11498 |
| 7355 | 11452 | 7402 | 11499 |
| 7356 | 11453 | 7403 | 11500 |
| 7357 | 11454 | 7404 | 11501 |
| 7358 | 11455 | 7405 | 11502 |
| 7359 | 11456 | 7406 | 11503 |
| 7360 | 11457 | 7407 | 11504 |
| 7361 | 11458 | 7408 | 11505 |
| 7362 | 11459 | 7409 | 11506 |
| 7363 | 11460 | 7410 | 11507 |
| 7364 | 11461 | 7411 | 11508 |
| 7365 | 11462 | 7412 | 11509 |
| 7366 | 11463 | 7413 | 11510 |
| 7367 | 11464 | 7414 | 11511 |
| 7368 | 11465 | 7415 | 11512 |
| 7416 | 11513 | 7463 | 11560 |
| 7417 | 11514 | 7464 | 11561 |
| 7418 | 11515 | 7465 | 11562 |
| 7419 | 11516 | 7466 | 11563 |
| 7420 | 11517 | 7467 | 11564 |
| 7421 | 11518 | 7468 | 11565 |
| 7422 | 11519 | 7469 | 11566 |
| 7423 | 11520 | 7470 | 11567 |
| 7424 | 11521 | 7471 | 11568 |
| 7425 | 11522 | 7472 | 11569 |
| 7426 | 11523 | 7473 | 11570 |
| 7427 | 11524 | 7474 | 11571 |
| 7428 | 11525 | 7475 | 11572 |
| 7429 | 11526 | 7476 | 11573 |
| 7430 | 11527 | 7477 | 11574 |
| 7431 | 11528 | 7478 | 11575 |
| 7432 | 11529 | 7479 | 11576 |
| 7433 | 11530 | 7480 | 11577 |
| 7434 | 11531 | 7481 | 11578 |
| 7435 | 11532 | 7482 | 11579 |
| 7436 | 11533 | 7483 | 11580 |
| 7437 | 11534 | 7484 | 11581 |
| 7438 | 11535 | 7485 | 11582 |
| 7439 | 11536 | 7486 | 11583 |
| 7440 | 11537 | 7487 | 11584 |
| 7441 | 11538 | 7488 | 11585 |
| 7442 | 11539 | 7489 | 11586 |
| 7443 | 11540 | 7490 | 11587 |
| 7444 | 11541 | 7491 | 11588 |
| 7445 | 11542 | 7492 | 11589 |
| 7446 | 11543 | 7493 | 11590 |
| 7447 | 11544 | 7494 | 11591 |
| 7448 | 11545 | 7495 | 11592 |
| 7449 | 11546 | 7496 | 11593 |
| 7450 | 11547 | 7497 | 11594 |
| 7451 | 11548 | 7498 | 11595 |
| 7452 | 11549 | 7499 | 11596 |
| 7453 | 11550 | 7500 | 11597 |
| 7454 | 11551 | 7501 | 11598 |
| 7455 | 11552 | 7502 | 11599 |
| 7456 | 11553 | 7503 | 11600 |
| 7457 | 11554 | 7504 | 11601 |
| 7458 | 11555 | 7505 | 11602 |
| 7459 | 11556 | 7506 | 11603 |
| 7460 | 11557 | 7507 | 11604 |
| 7461 | 11558 | 7508 | 11605 |
| 7462 | 11559 | 7509 | 11606 |
| 7510 | 11607 | 7557 | 11654 |
| 7511 | 11608 | 7558 | 11655 |
| 7512 | 11609 | 7559 | 11656 |
| 7513 | 11610 | 7560 | 11657 |
| 7514 | 11611 | 7561 | 11658 |
| 7515 | 11612 | 7562 | 11659 |
| 7516 | 11613 | 7563 | 11660 |
| 7517 | 11614 | 7564 | 11661 |
| 7518 | 11615 | 7565 | 11662 |
| 7519 | 11616 | 7566 | 11663 |
| 7520 | 11617 | 7567 | 11664 |
| 7521 | 11618 | 7568 | 11665 |
| 7522 | 11619 | 7569 | 11666 |
| 7523 | 11620 | 7570 | 11667 |
| 7524 | 11621 | 7571 | 11668 |
| 7525 | 11622 | 7572 | 11669 |
| 7526 | 11623 | 7573 | 11670 |
| 7527 | 11624 | 7574 | 11671 |
| 7528 | 11625 | 7575 | 11672 |
| 7529 | 11626 | 7576 | 11673 |
| 7530 | 11627 | 7577 | 11674 |
| 7531 | 11628 | 7578 | 11675 |
| 7532 | 11629 | 7579 | 11676 |
| 7533 | 11630 | 7580 | 11677 |
| 7534 | 11631 | 7581 | 11678 |
| 7535 | 11632 | 7582 | 11679 |
| 7536 | 11633 | 7583 | 11680 |
| 7537 | 11634 | 7584 | 11681 |
| 7538 | 11635 | 7585 | 11682 |
| 7539 | 11636 | 7586 | 11683 |
| 7540 | 11637 | 7587 | 11684 |
| 7541 | 11638 | 7588 | 11685 |
| 7542 | 11639 | 7589 | 11686 |
| 7543 | 11640 | 7590 | 11687 |
| 7544 | 11641 | 7591 | 11688 |
| 7545 | 11642 | 7592 | 11689 |
| 7546 | 11643 | 7593 | 11690 |
| 7547 | 11644 | 7594 | 11691 |
| 7548 | 11645 | 7595 | 11692 |
| 7549 | 11646 | 7596 | 11693 |
| 7550 | 11647 | 7597 | 11694 |
| 7551 | 11648 | 7598 | 11695 |
| 7552 | 11649 | 7599 | 11696 |
| 7553 | 11650 | 7600 | 11697 |
| 7554 | 11651 | 7601 | 11698 |
| 7555 | 11652 | 7602 | 11699 |
| 7556 | 11653 | 7603 | 11700 |
| 7604 | 11701 | 7651 | 11748 |
| 7605 | 11702 | 7652 | 11749 |
| 7606 | 11703 | 7653 | 11750 |
| 7607 | 11704 | 7654 | 11751 |
| 7608 | 11705 | 7655 | 11752 |
| 7609 | 11706 | 7656 | 11753 |
| 7610 | 11707 | 7657 | 11754 |
| 7611 | 11708 | 7658 | 11755 |
| 7612 | 11709 | 7659 | 11756 |
| 7613 | 11710 | 7660 | 11757 |
| 7614 | 11711 | 7661 | 11758 |
| 7615 | 11712 | 7662 | 11759 |
| 7616 | 11713 | 7663 | 11760 |
| 7617 | 11714 | 7664 | 11761 |
| 7618 | 11715 | 7665 | 11762 |
| 7619 | 11716 | 7666 | 11763 |
| 7620 | 11717 | 7667 | 11764 |
| 7621 | 11718 | 7668 | 11765 |
| 7622 | 11719 | 7669 | 11766 |
| 7623 | 11720 | 7670 | 11767 |
| 7624 | 11721 | 7671 | 11768 |
| 7625 | 11722 | 7672 | 11769 |
| 7626 | 11723 | 7673 | 11770 |
| 7627 | 11724 | 7674 | 11771 |
| 7628 | 11725 | 4052 | 5107 |
| 7629 | 11726 | 4053 | 5108 |
| 7630 | 11727 | 4054 | 5109 |
| 7631 | 11728 | 4055 | 5110 |
| 7632 | 11729 | 4056 | 5111 |
| 7633 | 11730 | 4057 | 5112 |
| 7634 | 11731 | 4058 | 5113 |
| 7635 | 11732 | 4059 | 5114 |
| 7636 | 11733 | 4060 | 5115 |
| 7637 | 11734 | 4061 | 5116 |
| 7638 | 11735 | 4062 | 5117 |
| 7639 | 11736 | 4063 | 5118 |
| 7640 | 11737 | 4064 | 5119 |
| 7641 | 11738 | 4065 | 5120 |
| 7642 | 11739 | 4066 | 5121 |
| 7643 | 11740 | 4067 | 5122 |
| 7644 | 11741 | 4068 | 5123 |
| 7645 | 11742 | 4069 | 5124 |
| 7646 | 11743 | 4070 | 5125 |
| 7647 | 11744 | 4071 | 5126 |
| 7648 | 11745 | 4072 | 5127 |
| 7649 | 11746 | 4073 | 5128 |
| 7650 | 11747 | 4074 | 5129 |
| 4075 | 5130 | 4122 | 5177 |
| 4076 | 5131 | 4123 | 5178 |
| 4077 | 5132 | 4124 | 5179 |
| 4078 | 5133 | 4125 | 5180 |
| 4079 | 5134 | 4126 | 5181 |
| 4080 | 5135 | 4127 | 5182 |
| 4081 | 5136 | 4128 | 5183 |
| 4082 | 5137 | 4129 | 5184 |
| 4083 | 5138 | 4130 | 5185 |
| 4084 | 5139 | 4131 | 5186 |
| 4085 | 5140 | 4132 | 5187 |
| 4086 | 5141 | 4133 | 5188 |
| 4087 | 5142 | 4134 | 5189 |
| 4088 | 5143 | 4135 | 5190 |
| 4089 | 5144 | 4136 | 5191 |
| 4090 | 5145 | 4137 | 5192 |
| 4091 | 5146 | 4138 | 5193 |
| 4092 | 5147 | 4139 | 5194 |
| 4093 | 5148 | 4140 | 5195 |
| 4094 | 5149 | 4141 | 5196 |
| 4095 | 5150 | 4142 | 5197 |
| 4096 | 5151 | 4143 | 5198 |
| 4097 | 5152 | 4144 | 5199 |
| 4098 | 5153 | 4145 | 5200 |
| 4099 | 5154 | 4146 | 5201 |
| 4100 | 5155 | 4147 | 5202 |
| 4101 | 5156 | 4148 | 5203 |
| 4102 | 5157 | 4149 | 5204 |
| 4103 | 5158 | 4150 | 5205 |
| 4104 | 5159 | 4151 | 5206 |
| 4105 | 5160 | 4152 | 5207 |
| 4106 | 5161 | 4153 | 5208 |
| 4107 | 5162 | 4154 | 5209 |
| 4108 | 5163 | 4155 | 5210 |
| 4109 | 5164 | 4156 | 5211 |
| 4110 | 5165 | 4157 | 5212 |
| 4111 | 5166 | 4158 | 5213 |
| 4112 | 5167 | 4159 | 5214 |
| 4113 | 5168 | 4160 | 5215 |
| 4114 | 5169 | 4161 | 5216 |
| 4115 | 5170 | 4162 | 5217 |
| 4116 | 5171 | 4163 | 5218 |
| 4117 | 5172 | 4164 | 5219 |
| 4118 | 5173 | 4165 | 5220 |
| 4119 | 5174 | 3998 | 5053 |
| 4120 | 5175 | 3999 | 5054 |
| 4121 | 5176 | 4000 | 5055 |
| 4001 | 5056 | 4048 | 5103 |
| 4002 | 5057 | 4049 | 5104 |
| 4003 | 5058 | 4050 | 5105 |
| 4004 | 5059 | 4051 | 5106 |
| 4005 | 5060 | 7675 | 11772 |
| 4006 | 5061 | 7676 | 11773 |
| 4007 | 5062 | 7677 | 11774 |
| 4008 | 5063 | 7678 | 11775 |
| 4009 | 5064 | 7676 | 11776 |
| 4010 | 5065 | 7679 | 11777 |
| 4011 | 5066 | 7680 | 11778 |
| 4012 | 5067 | 7681 | 11779 |
| 4013 | 5068 | 7682 | 11780 |
| 4014 | 5069 | 7683 | 11781 |
| 4015 | 5070 | 7684 | 11782 |
| 4016 | 5071 | 7685 | 11783 |
| 4017 | 5072 | 7686 | 11784 |
| 4018 | 5073 | 7687 | 11785 |
| 4019 | 5074 | 7688 | 11786 |
| 4020 | 5075 | 7689 | 11787 |
| 4021 | 5076 | 7690 | 11788 |
| 4022 | 5077 | 7691 | 11789 |
| 4023 | 5078 | 7692 | 11790 |
| 4024 | 5079 | 7693 | 11791 |
| 4025 | 5080 | 7694 | 11792 |
| 4026 | 5081 | 7695 | 11793 |
| 4027 | 5082 | 7696 | 11794 |
| 4028 | 5083 | 7697 | 11795 |
| 4029 | 5084 | 7698 | 11796 |
| 4030 | 5085 | 7699 | 11797 |
| 4031 | 5086 | 7700 | 11798 |
| 4032 | 5087 | 7701 | 11799 |
| 4033 | 5088 | 7702 | 11800 |
| 4034 | 5089 | 7703 | 11801 |
| 4035 | 5090 | 7704 | 11802 |
| 4036 | 5091 | 7705 | 11803 |
| 4037 | 5092 | 7706 | 11804 |
| 4038 | 5093 | 7707 | 11805 |
| 4039 | 5094 | 7708 | 11806 |
| 4040 | 5095 | 7709 | 11807 |
| 4041 | 5096 | 7710 | 11808 |
| 4042 | 5097 | 7711 | 11809 |
| 4043 | 5098 | 7712 | 11810 |
| 4044 | 5099 | 7713 | 11811 |
| 4045 | 5100 | 7714 | 11812 |
| 4046 | 5101 | 7715 | 11813 |
| 4047 | 5102 | 7716 | 11814 |
| 7717 | 11815 | 7764 | 11862 |
| 7718 | 11816 | 7765 | 11863 |
| 7719 | 11817 | 7766 | 11864 |
| 7720 | 11818 | 7767 | 11865 |
| 7721 | 11819 | 7768 | 11866 |
| 7722 | 11820 | 7769 | 11867 |
| 7723 | 11821 | 7770 | 11868 |
| 7724 | 11822 | 7771 | 11869 |
| 7725 | 11823 | 7772 | 11870 |
| 7726 | 11824 | 7773 | 11871 |
| 7727 | 11825 | 7774 | 11872 |
| 7728 | 11826 | 7775 | 11873 |
| 7729 | 11827 | 7776 | 11874 |
| 7730 | 11828 | 7777 | 11875 |
| 7731 | 11829 | 7778 | 11876 |
| 7732 | 11830 | 7779 | 11877 |
| 7733 | 11831 | 7780 | 11878 |
| 7734 | 11832 | 7781 | 11879 |
| 7735 | 11833 | 7782 | 11880 |
| 7736 | 11834 | 7783 | 11881 |
| 7737 | 11835 | 7784 | 11882 |
| 7738 | 11836 | 7785 | 11883 |
| 7739 | 11837 | 7786 | 11884 |
| 7740 | 11838 | 7787 | 11885 |
| 7741 | 11839 | 7788 | 11886 |
| 7742 | 11840 | 7789 | 11887 |
| 7743 | 11841 | 7790 | 11888 |
| 7744 | 11842 | 7791 | 11889 |
| 7745 | 11843 | 7792 | 11890 |
| 7746 | 11844 | 7793 | 11891 |
| 7747 | 11845 | 7794 | 11892 |
| 7748 | 11846 | 7795 | 11893 |
| 7749 | 11847 | 7796 | 11894 |
| 7750 | 11848 | 7797 | 11895 |
| 7751 | 11849 | 7798 | 11896 |
| 7752 | 11850 | 7799 | 11897 |
| 7753 | 11851 | 7800 | 11898 |
| 7754 | 11852 | 7801 | 11899 |
| 7755 | 11853 | 7802 | 11900 |
| 7756 | 11854 | 7803 | 11901 |
| 7757 | 11855 | 7804 | 11902 |
| 7758 | 11856 | 7805 | 11903 |
| 7759 | 11857 | 7806 | 11904 |
| 7760 | 11858 | 7807 | 11905 |
| 7761 | 11859 | 7808 | 11906 |
| 7762 | 11860 | 7809 | 11907 |
| 7763 | 11861 | 7810 | 11908 |
| 7811 | 11909 | 7858 | 11956 |
| 7812 | 11910 | 7859 | 11957 |
| 7813 | 11911 | 7860 | 11958 |
| 7814 | 11912 | 7861 | 11959 |
| 7815 | 11913 | 7862 | 11960 |
| 7816 | 11914 | 7863 | 11961 |
| 7817 | 11915 | 7864 | 11962 |
| 7818 | 11916 | 7865 | 11963 |
| 7819 | 11917 | 7866 | 11964 |
| 7820 | 11918 | 7867 | 11965 |
| 7821 | 11919 | 7868 | 11966 |
| 7822 | 11920 | 7869 | 11967 |
| 7823 | 11921 | 7870 | 11968 |
| 7824 | 11922 | 7871 | 11969 |
| 7825 | 11923 | 7872 | 11970 |
| 7826 | 11924 | 7873 | 11971 |
| 7827 | 11925 | 7874 | 11972 |
| 7828 | 11926 | 7875 | 11973 |
| 7829 | 11927 | 7876 | 11974 |
| 7830 | 11928 | 7877 | 11975 |
| 7831 | 11929 | 7878 | 11976 |
| 7832 | 11930 | 7879 | 11977 |
| 7833 | 11931 | 7880 | 11978 |
| 7834 | 11932 | 7881 | 11979 |
| 7835 | 11933 | 7882 | 11980 |
| 7836 | 11934 | 7883 | 11981 |
| 7837 | 11935 | 7884 | 11982 |
| 7838 | 11936 | 7885 | 11983 |
| 7839 | 11937 | 7886 | 11984 |
| 7840 | 11938 | 7887 | 11985 |
| 7841 | 11939 | 7888 | 11986 |
| 7842 | 11940 | 7889 | 11987 |
| 7843 | 11941 | 7890 | 11988 |
| 7844 | 11942 | 7891 | 11989 |
| 7845 | 11943 | 7892 | 11990 |
| 7846 | 11944 | 7893 | 11991 |
| 7847 | 11945 | 7894 | 11992 |
| 7848 | 11946 | 7895 | 11993 |
| 7849 | 11947 | 7896 | 11994 |
| 7850 | 11948 | 7897 | 11995 |
| 7851 | 11949 | 7898 | 11996 |
| 7852 | 11950 | 7899 | 11997 |
| 7853 | 11951 | 7900 | 11998 |
| 7854 | 11952 | 7901 | 11999 |
| 7855 | 11953 | 7902 | 12000 |
| 7856 | 11954 | 7903 | 12001 |
| 7857 | 11955 | 7904 | 12002 |
| 7905 | 12003 | 7952 | 12050 |
| 7906 | 12004 | 7953 | 12051 |
| 7907 | 12005 | 7954 | 12052 |
| 7908 | 12006 | 7955 | 12053 |
| 7909 | 12007 | 7956 | 12054 |
| 7910 | 12008 | 7957 | 12055 |
| 7911 | 12009 | 7958 | 12056 |
| 7912 | 12010 | 7959 | 12057 |
| 7913 | 12011 | 7960 | 12058 |
| 7914 | 12012 | 7961 | 12059 |
| 7915 | 12013 | 7962 | 12060 |
| 7916 | 12014 | 7963 | 12061 |
| 7917 | 12015 | 7964 | 12062 |
| 7918 | 12016 | 7965 | 12063 |
| 7919 | 12017 | 7966 | 12064 |
| 7920 | 12018 | 7967 | 12065 |
| 7921 | 12019 | 7968 | 12066 |
| 7922 | 12020 | 7969 | 12067 |
| 7923 | 12021 | 7970 | 12068 |
| 7924 | 12022 | 7971 | 12069 |
| 7925 | 12023 | 7972 | 12070 |
| 7926 | 12024 | 7973 | 12071 |
| 7927 | 12025 | 7974 | 12072 |
| 7928 | 12026 | 7975 | 12073 |
| 7929 | 12027 | 7976 | 12074 |
| 7930 | 12028 | 7977 | 12075 |
| 7931 | 12029 | 7978 | 12076 |
| 7932 | 12030 | 7979 | 12077 |
| 7933 | 12031 | 7980 | 12078 |
| 7934 | 12032 | 7981 | 12079 |
| 7935 | 12033 | 7982 | 12080 |
| 7936 | 12034 | 7983 | 12081 |
| 7937 | 12035 | 7984 | 12082 |
| 7938 | 12036 | 7985 | 12083 |
| 7939 | 12037 | 7986 | 12084 |
| 7940 | 12038 | 7987 | 12085 |
| 7941 | 12039 | 7988 | 12086 |
| 7942 | 12040 | 7989 | 12087 |
| 7943 | 12041 | 7990 | 12088 |
| 7944 | 12042 | 7991 | 12089 |
| 7945 | 12043 | 7992 | 12090 |
| 7946 | 12044 | 7993 | 12091 |
| 7947 | 12045 | 7994 | 12092 |
| 7948 | 12046 | 7995 | 12093 |
| 7949 | 12047 | 7996 | 12094 |
| 7950 | 12048 | 7997 | 12095 |
| 7951 | 12049 | 7998 | 12096 |
| 7999 | 12097 | 8046 | 12144 |
| 8000 | 12098 | 8047 | 12145 |
| 8001 | 12099 | 8048 | 12146 |
| 8002 | 12100 | 8049 | 12147 |
| 8003 | 12101 | 8050 | 12148 |
| 8004 | 12102 | 8051 | 12149 |
| 8005 | 12103 | 8052 | 12150 |
| 8006 | 12104 | 8053 | 12151 |
| 8007 | 12105 | 8054 | 12152 |
| 8008 | 12106 | 8055 | 12153 |
| 8009 | 12107 | 8056 | 12154 |
| 8010 | 12108 | 8057 | 12155 |
| 8011 | 12109 | 8058 | 12156 |
| 8012 | 12110 | 8059 | 12157 |
| 8013 | 12111 | 8060 | 12158 |
| 8014 | 12112 | 8061 | 12159 |
| 8015 | 12113 | 8062 | 12160 |
| 8016 | 12114 | 8063 | 12161 |
| 8017 | 12115 | 8064 | 12162 |
| 8018 | 12116 | 8065 | 12163 |
| 8019 | 12117 | 8066 | 12164 |
| 8020 | 12118 | 8067 | 12165 |
| 8021 | 12119 | 8068 | 12166 |
| 8022 | 12120 | 8069 | 12167 |
| 8023 | 12121 | 8070 | 12168 |
| 8024 | 12122 | 8071 | 12169 |
| 8025 | 12123 | 8072 | 12170 |
| 8026 | 12124 | 8073 | 12171 |
| 8027 | 12125 | 8074 | 12172 |
| 8028 | 12126 | 8075 | 12173 |
| 8029 | 12127 | 8076 | 12174 |
| 8030 | 12128 | 8077 | 12175 |
| 8031 | 12129 | 8078 | 12176 |
| 8032 | 12130 | 8079 | 12177 |
| 8033 | 12131 | 8080 | 12178 |
| 8034 | 12132 | 8081 | 12179 |
| 8035 | 12133 | 8082 | 12180 |
| 8036 | 12134 | 8083 | 12181 |
| 8037 | 12135 | 8084 | 12182 |
| 8038 | 12136 | 8085 | 12183 |
| 8039 | 12137 | 8086 | 12184 |
| 8040 | 12138 | 8087 | 12185 |
| 8041 | 12139 | 8088 | 12186 |
| 8042 | 12140 | 8089 | 12187 |
| 8043 | 12141 | 8090 | 12188 |
| 8044 | 12142 | 8091 | 12189 |
| 8045 | 12143 | 8092 | 12190 |
| 8093 | 12191 | 8140 | 12238 |
| 8094 | 12192 | 8141 | 12239 |
| 8095 | 12193 | 8142 | 12240 |
| 8096 | 12194 | 8143 | 12241 |
| 8097 | 12195 | 8144 | 12242 |
| 8098 | 12196 | 8145 | 12243 |
| 8099 | 12197 | 8146 | 12244 |
| 8100 | 12198 | 8147 | 12245 |
| 8101 | 12199 | 8148 | 12246 |
| 8102 | 12200 | 8149 | 12247 |
| 8103 | 12201 | 8150 | 12248 |
| 8104 | 12202 | 8151 | 12249 |
| 8105 | 12203 | 8152 | 12250 |
| 8106 | 12204 | 8153 | 12251 |
| 8107 | 12205 | 8154 | 12252 |
| 8108 | 12206 | 8155 | 12253 |
| 8109 | 12207 | 8156 | 12254 |
| 8110 | 12208 | 8157 | 12255 |
| 8111 | 12209 | 8158 | 12256 |
| 8112 | 12210 | 8159 | 12257 |
| 8113 | 12211 | 8160 | 12258 |
| 8114 | 12212 | 8161 | 12259 |
| 8115 | 12213 | 8162 | 12260 |
| 8116 | 12214 | 8163 | 12261 |
| 8117 | 12215 | 8164 | 12262 |
| 8118 | 12216 | 8165 | 12263 |
| 8119 | 12217 | 8166 | 12264 |
| 8120 | 12218 | 8167 | 12265 |
| 8121 | 12219 | 8168 | 12266 |
| 8122 | 12220 | 8169 | 12267 |
| 8123 | 12221 | 8170 | 12268 |
| 8124 | 12222 | 8171 | 12269 |
| 8125 | 12223 | 8172 | 12270 |
| 8126 | 12224 | 8173 | 12271 |
| 8127 | 12225 | 8174 | 12272 |
| 8128 | 12226 | 8175 | 12273 |
| 8129 | 12227 | 8176 | 12274 |
| 8130 | 12228 | 8177 | 12275 |
| 8131 | 12229 | 8178 | 12276 |
| 8132 | 12230 | 8179 | 12277 |
| 8133 | 12231 | 8180 | 12278 |
| 8134 | 12232 | 8181 | 12279 |
| 8135 | 12233 | 8182 | 12280 |
| 8136 | 12234 | 8183 | 12281 |
| 8137 | 12235 | 8184 | 12282 |
| 8138 | 12236 | 8185 | 12283 |
| 8139 | 12237 | 8186 | 12284 |
| 8187 | 12285 | 8234 | 12332 |
| 8188 | 12286 | 8235 | 12333 |
| 8189 | 12287 | 8236 | 12334 |
| 8190 | 12288 | 8237 | 12335 |
| 8191 | 12289 | 8238 | 12336 |
| 8192 | 12290 | 8239 | 12337 |
| 8193 | 12291 | 8240 | 12338 |
| 8194 | 12292 | 8241 | 12339 |
| 8195 | 12293 | 8242 | 12340 |
| 8196 | 12294 | 8243 | 12341 |
| 8197 | 12295 | 8244 | 12342 |
| 8198 | 12296 | 8245 | 12343 |
| 8199 | 12297 | 8246 | 12344 |
| 8200 | 12298 | 8247 | 12345 |
| 8201 | 12299 | 8248 | 12346 |
| 8202 | 12300 | 8249 | 12347 |
| 8203 | 12301 | 8250 | 12348 |
| 8204 | 12302 | 8251 | 12349 |
| 8205 | 12303 | 8252 | 12350 |
| 8206 | 12304 | 8253 | 12351 |
| 8207 | 12305 | 8254 | 12352 |
| 8208 | 12306 | 8255 | 12353 |
| 8209 | 12307 | 8256 | 12354 |
| 8210 | 12308 | 8257 | 12355 |
| 8211 | 12309 | 8258 | 12356 |
| 8212 | 12310 | 8259 | 12357 |
| 8213 | 12311 | 8260 | 12358 |
| 8214 | 12312 | 8261 | 12359 |
| 8215 | 12313 | 8262 | 12360 |
| 8216 | 12314 | 8263 | 12361 |
| 8217 | 12315 | 8264 | 12362 |
| 8218 | 12316 | 8265 | 12363 |
| 8219 | 12317 | 8266 | 12364 |
| 8220 | 12318 | 8267 | 12365 |
| 8221 | 12319 | 8268 | 12366 |
| 8222 | 12320 | 8269 | 12367 |
| 8223 | 12321 | 8270 | 12368 |
| 8224 | 12322 | 8271 | 12369 |
| 8225 | 12323 | 8272 | 12370 |
| 8226 | 12324 | 8273 | 12371 |
| 8227 | 12325 | 8274 | 12372 |
| 8228 | 12326 | 8275 | 12373 |
| 8229 | 12327 | 8276 | 12374 |
| 8230 | 12328 | 8277 | 12375 |
| 8231 | 12329 | 8278 | 12376 |
| 8232 | 12330 | 8279 | 12377 |
| 8233 | 12331 | 8280 | 12378 |
| 8281 | 12379 | 8328 | 12426 |
| 8282 | 12380 | 8329 | 12427 |
| 8283 | 12381 | 8330 | 12428 |
| 8284 | 12382 | 8331 | 12429 |
| 8285 | 12383 | 8332 | 12430 |
| 8286 | 12384 | 8333 | 12431 |
| 8287 | 12385 | 8334 | 12432 |
| 8288 | 12386 | 8335 | 12433 |
| 8289 | 12387 | 8336 | 12434 |
| 8290 | 12388 | 8337 | 12435 |
| 8291 | 12389 | 8338 | 12436 |
| 8292 | 12390 | 8339 | 12437 |
| 8293 | 12391 | 8340 | 12438 |
| 8294 | 12392 | 8341 | 12439 |
| 8295 | 12393 | 8342 | 12440 |
| 8296 | 12394 | 8343 | 12441 |
| 8297 | 12395 | 8344 | 12442 |
| 8298 | 12396 | 8345 | 12443 |
| 8299 | 12397 | 8346 | 12444 |
| 8300 | 12398 | 8347 | 12445 |
| 8301 | 12399 | 8348 | 12446 |
| 8302 | 12400 | 8349 | 12447 |
| 8303 | 12401 | 8350 | 12448 |
| 8304 | 12402 | 8351 | 12449 |
| 8305 | 12403 | 8352 | 12450 |
| 8306 | 12404 | 8353 | 12451 |
| 8307 | 12405 | 8354 | 12452 |
| 8308 | 12406 | 8355 | 12453 |
| 8309 | 12407 | 8356 | 12454 |
| 8310 | 12408 | 8357 | 12455 |
| 8311 | 12409 | 8358 | 12456 |
| 8312 | 12410 | 8359 | 12457 |
| 8313 | 12411 | 8360 | 12458 |
| 8314 | 12412 | 8361 | 12459 |
| 8315 | 12413 | 8362 | 12460 |
| 8316 | 12414 | 8363 | 12461 |
| 8317 | 12415 | 8364 | 12462 |
| 8318 | 12416 | 8365 | 12463 |
| 8319 | 12417 | 8366 | 12464 |
| 8320 | 12418 | 8367 | 12465 |
| 8321 | 12419 | 8368 | 12466 |
| 8322 | 12420 | 8369 | 12467 |
| 8323 | 12421 | 8370 | 12468 |
| 8324 | 12422 | 8371 | 12469 |
| 8325 | 12423 | 8372 | 12470 |
| 8326 | 12424 | 8373 | 12471 |
| 8327 | 12425 | 8374 | 12472 |
| 8375 | 12473 | 8422 | 12520 |
| 8376 | 12474 | 8423 | 12521 |
| 8377 | 12475 | 8424 | 12522 |
| 8378 | 12476 | 8425 | 12523 |
| 8379 | 12477 | 8426 | 12524 |
| 8380 | 12478 | 8427 | 12525 |
| 8381 | 12479 | 8428 | 12526 |
| 8382 | 12480 | 8429 | 12527 |
| 8383 | 12481 | 8430 | 12528 |
| 8384 | 12482 | 8431 | 12529 |
| 8385 | 12483 | 8432 | 12530 |
| 8386 | 12484 | 8433 | 12531 |
| 8387 | 12485 | 8434 | 12532 |
| 8388 | 12486 | 8435 | 12533 |
| 8389 | 12487 | 8436 | 12534 |
| 8390 | 12488 | 8437 | 12535 |
| 8391 | 12489 | 8438 | 12536 |
| 8392 | 12490 | 8439 | 12537 |
| 8393 | 12491 | 8440 | 12538 |
| 8394 | 12492 | 8441 | 12539 |
| 8395 | 12493 | 8442 | 12540 |
| 8396 | 12494 | 8443 | 12541 |
| 8397 | 12495 | 8444 | 12542 |
| 8398 | 12496 | 8445 | 12543 |
| 8399 | 12497 | 8446 | 12544 |
| 8400 | 12498 | 8447 | 12545 |
| 8401 | 12499 | 8448 | 12546 |
| 8402 | 12500 | 8449 | 12547 |
| 8403 | 12501 | 8450 | 12548 |
| 8404 | 12502 | 8451 | 12549 |
| 8405 | 12503 | 8452 | 12550 |
| 8406 | 12504 | 8453 | 12551 |
| 8407 | 12505 | 8454 | 12552 |
| 8408 | 12506 | 8455 | 12553 |
| 8409 | 12507 | 8456 | 12554 |
| 8410 | 12508 | 8457 | 12555 |
| 8411 | 12509 | 8458 | 12556 |
| 8412 | 12510 | 8459 | 12557 |
| 8413 | 12511 | 8460 | 12558 |
| 8414 | 12512 | 8461 | 12559 |
| 8415 | 12513 | 8462 | 12560 |
| 8416 | 12514 | 8463 | 12561 |
| 8417 | 12515 | 8464 | 12562 |
| 8418 | 12516 | 8465 | 12563 |
| 8419 | 12517 | 8466 | 12564 |
| 8420 | 12518 | 8467 | 12565 |
| 8421 | 12519 | 8468 | 12566 |
| 8469 | 12567 | 8516 | 12614 |
| 8470 | 12568 | 8517 | 12615 |
| 8471 | 12569 | 8518 | 12616 |
| 8472 | 12570 | 8519 | 12617 |
| 8473 | 12571 | 8520 | 12618 |
| 8474 | 12572 | 8521 | 12619 |
| 8475 | 12573 | 8522 | 12620 |
| 8476 | 12574 | 8523 | 12621 |
| 8477 | 12575 | 8524 | 12622 |
| 8478 | 12576 | 8525 | 12623 |
| 8479 | 12577 | 8526 | 12624 |
| 8480 | 12578 | 8527 | 12625 |
| 8481 | 12579 | 8528 | 12626 |
| 8482 | 12580 | 8529 | 12627 |
| 8483 | 12581 | 8530 | 12628 |
| 8484 | 12582 | 8531 | 12629 |
| 8485 | 12583 | 8532 | 12630 |
| 8486 | 12584 | 8533 | 12631 |
| 8487 | 12585 | 8534 | 12632 |
| 8488 | 12586 | 8535 | 12633 |
| 8489 | 12587 | 8536 | 12634 |
| 8490 | 12588 | 8537 | 12635 |
| 8491 | 12589 | 8538 | 12636 |
| 8492 | 12590 | 8539 | 12637 |
| 8493 | 12591 | 8540 | 12638 |
| 8494 | 12592 | 8541 | 12639 |
| 8495 | 12593 | 8542 | 12640 |
| 8496 | 12594 | 8543 | 12641 |
| 8497 | 12595 | 8544 | 12642 |
| 8498 | 12596 | 8545 | 12643 |
| 8499 | 12597 | 8546 | 12644 |
| 8500 | 12598 | 8547 | 12645 |
| 8501 | 12599 | 8548 | 12646 |
| 8502 | 12600 | 8549 | 12647 |
| 8503 | 12601 | 8550 | 12648 |
| 8504 | 12602 | 8551 | 12649 |
| 8505 | 12603 | 8552 | 12650 |
| 8506 | 12604 | 8553 | 12651 |
| 8507 | 12605 | 8554 | 12652 |
| 8508 | 12606 | 8555 | 12653 |
| 8509 | 12607 | 8556 | 12654 |
| 8510 | 12608 | 8557 | 12655 |
| 8511 | 12609 | 8558 | 12656 |
| 8512 | 12610 | 8559 | 12657 |
| 8513 | 12611 | 8560 | 12658 |
| 8514 | 12612 | 8561 | 12659 |
| 8515 | 12613 | 8562 | 12660 |
| 8563 | 12661 | 8610 | 12708 |
| 8564 | 12662 | 8611 | 12709 |
| 8565 | 12663 | 8612 | 12710 |
| 8566 | 12664 | 8613 | 12711 |
| 8567 | 12665 | 8614 | 12712 |
| 8568 | 12666 | 8615 | 12713 |
| 8569 | 12667 | 8616 | 12714 |
| 8570 | 12668 | 8617 | 12715 |
| 8571 | 12669 | 8618 | 12716 |
| 8572 | 12670 | 8619 | 12717 |
| 8573 | 12671 | 8620 | 12718 |
| 8574 | 12672 | 8621 | 12719 |
| 8575 | 12673 | 8622 | 12720 |
| 8576 | 12674 | 8623 | 12721 |
| 8577 | 12675 | 8624 | 12722 |
| 8578 | 12676 | 8625 | 12723 |
| 8579 | 12677 | 8626 | 12724 |
| 8580 | 12678 | 8627 | 12725 |
| 8581 | 12679 | 8628 | 12726 |
| 8582 | 12680 | 8629 | 12727 |
| 8583 | 12681 | 8630 | 12728 |
| 8584 | 12682 | 8631 | 12729 |
| 8585 | 12683 | 8632 | 12730 |
| 8586 | 12684 | 8633 | 12731 |
| 8587 | 12685 | 8634 | 12732 |
| 8588 | 12686 | 8635 | 12733 |
| 8589 | 12687 | 8636 | 12734 |
| 8590 | 12688 | 8637 | 12735 |
| 8591 | 12689 | 8638 | 12736 |
| 8592 | 12690 | 8639 | 12737 |
| 8593 | 12691 | 8640 | 12738 |
| 8594 | 12692 | 8641 | 12739 |
| 8595 | 12693 | 8642 | 12740 |
| 8596 | 12694 | 8643 | 12741 |
| 8597 | 12695 | 8644 | 12742 |
| 8598 | 12696 | 8645 | 12743 |
| 8599 | 12697 | 8646 | 12744 |
| 8600 | 12698 | 8647 | 12745 |
| 8601 | 12699 | 8648 | 12746 |
| 8602 | 12700 | 8649 | 12747 |
| 8603 | 12701 | 8650 | 12748 |
| 8604 | 12702 | 8651 | 12749 |
| 8605 | 12703 | 8652 | 12750 |
| 8606 | 12704 | 8653 | 12751 |
| 8607 | 12705 | 8654 | 12752 |
| 8608 | 12706 | 8655 | 12753 |
| 8609 | 12707 | 8656 | 12754 |
| 8657 | 12755 | 8704 | 12802 |
| 8658 | 12756 | 8705 | 12803 |
| 8659 | 12757 | 8706 | 12804 |
| 8660 | 12758 | 8707 | 12805 |
| 8661 | 12759 | 8708 | 12806 |
| 8662 | 12760 | 8709 | 12807 |
| 8663 | 12761 | 8710 | 12808 |
| 8664 | 12762 | 8711 | 12809 |
| 8665 | 12763 | 8712 | 12810 |
| 8666 | 12764 | 8713 | 12811 |
| 8667 | 12765 | 8714 | 12812 |
| 8668 | 12766 | 8715 | 12813 |
| 8669 | 12767 | 8716 | 12814 |
| 8670 | 12768 | 8717 | 12815 |
| 8671 | 12769 | 8718 | 12816 |
| 8672 | 12770 | 8719 | 12817 |
| 8673 | 12771 | 8720 | 12818 |
| 8674 | 12772 | 8721 | 12819 |
| 8675 | 12773 | 8722 | 12820 |
| 8676 | 12774 | 8723 | 12821 |
| 8677 | 12775 | 8724 | 12822 |
| 8678 | 12776 | 8725 | 12823 |
| 8679 | 12777 | 8726 | 12824 |
| 8680 | 12778 | 8727 | 12825 |
| 8681 | 12779 | 8728 | 12826 |
| 8682 | 12780 | 8729 | 12827 |
| 8683 | 12781 | 8730 | 12828 |
| 8684 | 12782 | 8731 | 12829 |
| 8685 | 12783 | 8732 | 12830 |
| 8686 | 12784 | 8733 | 12831 |
| 8687 | 12785 | 8734 | 12832 |
| 8688 | 12786 | 8735 | 12833 |
| 8689 | 12787 | 8736 | 12834 |
| 8690 | 12788 | 8737 | 12835 |
| 8691 | 12789 | 8738 | 12836 |
| 8692 | 12790 | 8739 | 12837 |
| 8693 | 12791 | 8740 | 12838 |
| 8694 | 12792 | 8741 | 12839 |
| 8695 | 12793 | 8742 | 12840 |
| 8696 | 12794 | 8743 | 12841 |
| 8697 | 12795 | 8744 | 12842 |
| 8698 | 12796 | 8745 | 12843 |
| 8699 | 12797 | 8746 | 12844 |
| 8700 | 12798 | 8747 | 12845 |
| 8701 | 12799 | 8748 | 12846 |
| 8702 | 12800 | 8749 | 12847 |
| 8703 | 12801 | 8750 | 12848 |
| 8751 | 12849 | 8798 | 12896 |
| 8752 | 12850 | 8799 | 12897 |
| 8753 | 12851 | 8800 | 12898 |
| 8754 | 12852 | 8801 | 12899 |
| 8755 | 12853 | 8802 | 12900 |
| 8756 | 12854 | 8803 | 12901 |
| 8757 | 12855 | 8804 | 12902 |
| 8758 | 12856 | 8805 | 12903 |
| 8759 | 12857 | 8806 | 12904 |
| 8760 | 12858 | 8807 | 12905 |
| 8761 | 12859 | 8808 | 12906 |
| 8762 | 12860 | 8809 | 12907 |
| 8763 | 12861 | 8810 | 12908 |
| 8764 | 12862 | 8811 | 12909 |
| 8765 | 12863 | 8812 | 12910 |
| 8766 | 12864 | 8813 | 12911 |
| 8767 | 12865 | 8814 | 12912 |
| 8768 | 12866 | 8815 | 12913 |
| 8769 | 12867 | 8816 | 12914 |
| 8770 | 12868 | 8817 | 12915 |
| 8771 | 12869 | 8818 | 12916 |
| 8772 | 12870 | 8819 | 12917 |
| 8773 | 12871 | 8820 | 12918 |
| 8774 | 12872 | 8821 | 12919 |
| 8775 | 12873 | 8822 | 12920 |
| 8776 | 12874 | 8823 | 12921 |
| 8777 | 12875 | 8824 | 12922 |
| 8778 | 12876 | 8825 | 12923 |
| 8779 | 12877 | 8826 | 12924 |
| 8780 | 12878 | 8827 | 12925 |
| 8781 | 12879 | 8828 | 12926 |
| 8782 | 12880 | 8829 | 12927 |
| 8783 | 12881 | 8830 | 12928 |
| 8784 | 12882 | 8831 | 12929 |
| 8785 | 12883 | 8832 | 12930 |
| 8786 | 12884 | 8833 | 12931 |
| 8787 | 12885 | 8834 | 12932 |
| 8788 | 12886 | 8835 | 12933 |
| 8789 | 12887 | 8836 | 12934 |
| 8790 | 12888 | 8837 | 12935 |
| 8791 | 12889 | 8838 | 12936 |
| 8792 | 12890 | 8839 | 12937 |
| 8793 | 12891 | 8840 | 12938 |
| 8794 | 12892 | 8841 | 12939 |
| 8795 | 12893 | 8842 | 12940 |
| 8796 | 12894 | 8843 | 12941 |
| 8797 | 12895 | 8844 | 12942 |
| 8845 | 12943 | 8892 | 12990 |
| 8846 | 12944 | 8893 | 12991 |
| 8847 | 12945 | 8894 | 12992 |
| 8848 | 12946 | 8895 | 12993 |
| 8849 | 12947 | 8896 | 12994 |
| 8850 | 12948 | 8897 | 12995 |
| 8851 | 12949 | 8898 | 12996 |
| 8852 | 12950 | 8899 | 12997 |
| 8853 | 12951 | 8900 | 12998 |
| 8854 | 12952 | 8901 | 12999 |
| 8855 | 12953 | 8902 | 13000 |
| 8856 | 12954 | 8903 | 13001 |
| 8857 | 12955 | 8904 | 13002 |
| 8858 | 12956 | 8905 | 13003 |
| 8859 | 12957 | 8906 | 13004 |
| 8860 | 12958 | 8907 | 13005 |
| 8861 | 12959 | 8908 | 13006 |
| 8862 | 12960 | 8909 | 13007 |
| 8863 | 12961 | 8910 | 13008 |
| 8864 | 12962 | 8911 | 13009 |
| 8865 | 12963 | 8912 | 13010 |
| 8866 | 12964 | 8913 | 13011 |
| 8867 | 12965 | 8914 | 13012 |
| 8868 | 12966 | 8915 | 13013 |
| 8869 | 12967 | 8916 | 13014 |
| 8870 | 12968 | 8917 | 13015 |
| 8871 | 12969 | 8918 | 13016 |
| 8872 | 12970 | 8919 | 13017 |
| 8873 | 12971 | 8920 | 13018 |
| 8874 | 12972 | 8921 | 13019 |
| 8875 | 12973 | 8922 | 13020 |
| 8876 | 12974 | 8923 | 13021 |
| 8877 | 12975 | 8924 | 13022 |
| 8878 | 12976 | 8925 | 13023 |
| 8879 | 12977 | 8926 | 13024 |
| 8880 | 12978 | 8927 | 13025 |
| 8881 | 12979 | 8928 | 13026 |
| 8882 | 12980 | 8929 | 13027 |
| 8883 | 12981 | 8930 | 13028 |
| 8884 | 12982 | 8931 | 13029 |
| 8885 | 12983 | 8932 | 13030 |
| 8886 | 12984 | 8933 | 13031 |
| 8887 | 12985 | 8934 | 13032 |
| 8888 | 12986 | 8935 | 13033 |
| 8889 | 12987 | 8936 | 13034 |
| 8890 | 12988 | 8937 | 13035 |
| 8891 | 12989 | 8938 | 13036 |
| 8939 | 13037 | 8986 | 13084 |
| 8940 | 13038 | 8987 | 13085 |
| 8941 | 13039 | 8988 | 13086 |
| 8942 | 13040 | 8989 | 13087 |
| 8943 | 13041 | 8990 | 13088 |
| 8944 | 13042 | 8991 | 13089 |
| 8945 | 13043 | 8992 | 13090 |
| 8946 | 13044 | 8993 | 13091 |
| 8947 | 13045 | 8994 | 13092 |
| 8948 | 13046 | 8995 | 13093 |
| 8949 | 13047 | 8996 | 13094 |
| 8950 | 13048 | 8997 | 13095 |
| 8951 | 13049 | 8998 | 13096 |
| 8952 | 13050 | 8999 | 13097 |
| 8953 | 13051 | 9000 | 13098 |
| 8954 | 13052 | 9001 | 13099 |
| 8955 | 13053 | 9002 | 13100 |
| 8956 | 13054 | 9003 | 13101 |
| 8957 | 13055 | 9004 | 13102 |
| 8958 | 13056 | 9005 | 13103 |
| 8959 | 13057 | 9006 | 13104 |
| 8960 | 13058 | 9007 | 13105 |
| 8961 | 13059 | 9008 | 13106 |
| 8962 | 13060 | 9009 | 13107 |
| 8963 | 13061 | 9010 | 13108 |
| 8964 | 13062 | 9011 | 13109 |
| 8965 | 13063 | 9012 | 13110 |
| 8966 | 13064 | 9013 | 13111 |
| 8967 | 13065 | 9014 | 13112 |
| 8968 | 13066 | 9015 | 13113 |
| 8969 | 13067 | 9016 | 13114 |
| 8970 | 13068 | 9017 | 13115 |
| 8971 | 13069 | 9018 | 13116 |
| 8972 | 13070 | 9019 | 13117 |
| 8973 | 13071 | 9020 | 13118 |
| 8974 | 13072 | 9021 | 13119 |
| 8975 | 13073 | 9022 | 13120 |
| 8976 | 13074 | 9023 | 13121 |
| 8977 | 13075 | 9024 | 13122 |
| 8978 | 13076 | 9025 | 13123 |
| 8979 | 13077 | 9026 | 13124 |
| 8980 | 13078 | 9027 | 13125 |
| 8981 | 13079 | 9028 | 13126 |
| 8982 | 13080 | 9029 | 13127 |
| 8983 | 13081 | 9030 | 13128 |
| 8984 | 13082 | 9031 | 13129 |
| 8985 | 13083 | 9032 | 13130 |
| 9033 | 13131 | 9080 | 13178 |
| 9034 | 13132 | 9081 | 13179 |
| 9035 | 13133 | 9082 | 13180 |
| 9036 | 13134 | 9083 | 13181 |
| 9037 | 13135 | 9084 | 13182 |
| 9038 | 13136 | 9085 | 13183 |
| 9039 | 13137 | 9086 | 13184 |
| 9040 | 13138 | 9087 | 13185 |
| 9041 | 13139 | 9088 | 13186 |
| 9042 | 13140 | 9089 | 13187 |
| 9043 | 13141 | 9090 | 13188 |
| 9044 | 13142 | 9091 | 13189 |
| 9045 | 13143 | 9092 | 13190 |
| 9046 | 13144 | 9093 | 13191 |
| 9047 | 13145 | 9094 | 13192 |
| 9048 | 13146 | 9095 | 13193 |
| 9049 | 13147 | 9096 | 13194 |
| 9050 | 13148 | 9097 | 13195 |
| 9051 | 13149 | 9098 | 13196 |
| 9052 | 13150 | 9099 | 13197 |
| 9053 | 13151 | 9100 | 13198 |
| 9054 | 13152 | 9101 | 13199 |
| 9055 | 13153 | 9102 | 13200 |
| 9056 | 13154 | 9103 | 13201 |
| 9057 | 13155 | 9104 | 13202 |
| 9058 | 13156 | 9105 | 13203 |
| 9059 | 13157 | 9106 | 13204 |
| 9060 | 13158 | 9107 | 13205 |
| 9061 | 13159 | 9108 | 13206 |
| 9062 | 13160 | 9109 | 13207 |
| 9063 | 13161 | 9110 | 13208 |
| 9064 | 13162 | 9111 | 13209 |
| 9065 | 13163 | 9112 | 13210 |
| 9066 | 13164 | 9113 | 13211 |
| 9067 | 13165 | 9114 | 13212 |
| 9068 | 13166 | 9115 | 13213 |
| 9069 | 13167 | 9116 | 13214 |
| 9070 | 13168 | 9117 | 13215 |
| 9071 | 13169 | 9118 | 13216 |
| 9072 | 13170 | 9119 | 13217 |
| 9073 | 13171 | 9120 | 13218 |
| 9074 | 13172 | 9121 | 13219 |
| 9075 | 13173 | 9122 | 13220 |
| 9076 | 13174 | 9123 | 13221 |
| 9077 | 13175 | 9124 | 13222 |
| 9078 | 13176 | 9125 | 13223 |
| 9079 | 13177 | 9126 | 13224 |
| 9127 | 13225 | 9174 | 13272 |
| 9128 | 13226 | 9175 | 13273 |
| 9129 | 13227 | 9176 | 13274 |
| 9130 | 13228 | 9177 | 13275 |
| 9131 | 13229 | 9178 | 13276 |
| 9132 | 13230 | 9179 | 13277 |
| 9133 | 13231 | 9180 | 13278 |
| 9134 | 13232 | 9181 | 13279 |
| 9135 | 13233 | 9182 | 13280 |
| 9136 | 13234 | 9183 | 13281 |
| 9137 | 13235 | 9184 | 13282 |
| 9138 | 13236 | 9185 | 13283 |
| 9139 | 13237 | 9186 | 13284 |
| 9140 | 13238 | 9187 | 13285 |
| 9141 | 13239 | 9188 | 13286 |
| 9142 | 13240 | 9189 | 13287 |
| 9143 | 13241 | 9190 | 13288 |
| 9144 | 13242 | 9191 | 13289 |
| 9145 | 13243 | 9192 | 13290 |
| 9146 | 13244 | 9193 | 13291 |
| 9147 | 13245 | 9194 | 13292 |
| 9148 | 13246 | 9195 | 13293 |
| 9149 | 13247 | 9196 | 13294 |
| 9150 | 13248 | 9197 | 13295 |
| 9151 | 13249 | 9198 | 13296 |
| 9152 | 13250 | 9199 | 13297 |
| 9153 | 13251 | 9200 | 13298 |
| 9154 | 13252 | 9201 | 13299 |
| 9155 | 13253 | 9202 | 13300 |
| 9156 | 13254 | 9203 | 13301 |
| 9157 | 13255 | 9204 | 13302 |
| 9158 | 13256 | 9205 | 13303 |
| 9159 | 13257 | 9206 | 13304 |
| 9160 | 13258 | 9207 | 13305 |
| 9161 | 13259 | 9208 | 13306 |
| 9162 | 13260 | 9209 | 13307 |
| 9163 | 13261 | 9210 | 13308 |
| 9164 | 13262 | 9211 | 13309 |
| 9165 | 13263 | 9212 | 13310 |
| 9166 | 13264 | 9213 | 13311 |
| 9167 | 13265 | 9214 | 13312 |
| 9168 | 13266 | 9215 | 13313 |
| 9169 | 13267 | 9216 | 13314 |
| 9170 | 13268 | 9217 | 13315 |
| 9171 | 13269 | 9218 | 13316 |
| 9172 | 13270 | 9219 | 13317 |
| 9173 | 13271 | 9220 | 13318 |
| 9221 | 13319 | 9268 | 13366 |
| 9222 | 13320 | 9269 | 13367 |
| 9223 | 13321 | 9270 | 13368 |
| 9224 | 13322 | 9271 | 13369 |
| 9225 | 13323 | 9272 | 13370 |
| 9226 | 13324 | 9273 | 13371 |
| 9227 | 13325 | 9274 | 13372 |
| 9228 | 13326 | 9275 | 13373 |
| 9229 | 13327 | 9276 | 13374 |
| 9230 | 13328 | 9277 | 13375 |
| 9231 | 13329 | 9278 | 13376 |
| 9232 | 13330 | 9279 | 13377 |
| 9233 | 13331 | 9280 | 13378 |
| 9234 | 13332 | 9281 | 13379 |
| 9235 | 13333 | 9282 | 13380 |
| 9236 | 13334 | 9283 | 13381 |
| 9237 | 13335 | 9284 | 13382 |
| 9238 | 13336 | 9285 | 13383 |
| 9239 | 13337 | 9286 | 13384 |
| 9240 | 13338 | 9287 | 13385 |
| 9241 | 13339 | 9288 | 13386 |
| 9242 | 13340 | 9289 | 13387 |
| 9243 | 13341 | 9290 | 13388 |
| 9244 | 13342 | 9291 | 13389 |
| 9245 | 13343 | 9292 | 13390 |
| 9246 | 13344 | 9293 | 13391 |
| 9247 | 13345 | 9294 | 13392 |
| 9248 | 13346 | 9295 | 13393 |
| 9249 | 13347 | 9296 | 13394 |
| 9250 | 13348 | 9297 | 13395 |
| 9251 | 13349 | 9298 | 13396 |
| 9252 | 13350 | 9299 | 13397 |
| 9253 | 13351 | 9300 | 13398 |
| 9254 | 13352 | 9301 | 13399 |
| 9255 | 13353 | 9302 | 13400 |
| 9256 | 13354 | 9303 | 13401 |
| 9257 | 13355 | 9304 | 13402 |
| 9258 | 13356 | 9305 | 13403 |
| 9259 | 13357 | 9306 | 13404 |
| 9260 | 13358 | 9307 | 13405 |
| 9261 | 13359 | 9308 | 13406 |
| 9262 | 13360 | 9309 | 13407 |
| 9263 | 13361 | 9310 | 13408 |
| 9264 | 13362 | 9311 | 13409 |
| 9265 | 13363 | 9312 | 13410 |
| 9266 | 13364 | 9313 | 13411 |
| 9267 | 13365 | 9314 | 13412 |
| 9315 | 13413 | 9362 | 13460 |
| 9316 | 13414 | 9363 | 13461 |
| 9317 | 13415 | 9364 | 13462 |
| 9318 | 13416 | 9365 | 13463 |
| 9319 | 13417 | 9366 | 13464 |
| 9320 | 13418 | 9367 | 13465 |
| 9321 | 13419 | 9368 | 13466 |
| 9322 | 13420 | 9369 | 13467 |
| 9323 | 13421 | 9370 | 13468 |
| 9324 | 13422 | 9371 | 13469 |
| 9325 | 13423 | 9372 | 13470 |
| 9326 | 13424 | 9373 | 13471 |
| 9327 | 13425 | 9374 | 13472 |
| 9328 | 13426 | 9375 | 13473 |
| 9329 | 13427 | 9376 | 13474 |
| 9330 | 13428 | 9377 | 13475 |
| 9331 | 13429 | 9378 | 13476 |
| 9332 | 13430 | 9379 | 13477 |
| 9333 | 13431 | 9380 | 13478 |
| 9334 | 13432 | 9381 | 13479 |
| 9335 | 13433 | 9382 | 13480 |
| 9336 | 13434 | 9383 | 13481 |
| 9337 | 13435 | 9384 | 13482 |
| 9338 | 13436 | 9385 | 13483 |
| 9339 | 13437 | 9386 | 13484 |
| 9340 | 13438 | 9387 | 13485 |
| 9341 | 13439 | 9388 | 13486 |
| 9342 | 13440 | 9389 | 13487 |
| 9343 | 13441 | 9390 | 13488 |
| 9344 | 13442 | 9391 | 13489 |
| 9345 | 13443 | 9392 | 13490 |
| 9346 | 13444 | 9393 | 13491 |
| 9347 | 13445 | 9394 | 13492 |
| 9348 | 13446 | 9395 | 13493 |
| 9349 | 13447 | 9396 | 13494 |
| 9350 | 13448 | 9397 | 13495 |
| 9351 | 13449 | 9398 | 13496 |
| 9352 | 13450 | 9399 | 13497 |
| 9353 | 13451 | 9400 | 13498 |
| 9354 | 13452 | 9401 | 13499 |
| 9355 | 13453 | 9402 | 13500 |
| 9356 | 13454 | 9403 | 13501 |
| 9357 | 13455 | 9404 | 13502 |
| 9358 | 13456 | 9405 | 13503 |
| 9359 | 13457 | 9406 | 13504 |
| 9360 | 13458 | 9407 | 13505 |
| 9361 | 13459 | 9408 | 13506 |
| 9409 | 13507 | 9456 | 13554 |
| 9410 | 13508 | 9457 | 13555 |
| 9411 | 13509 | 9458 | 13556 |
| 9412 | 13510 | 9459 | 13557 |
| 9413 | 13511 | 9460 | 13558 |
| 9414 | 13512 | 9461 | 13559 |
| 9415 | 13513 | 9462 | 13560 |
| 9416 | 13514 | 9463 | 13561 |
| 9417 | 13515 | 9464 | 13562 |
| 9418 | 13516 | 9465 | 13563 |
| 9419 | 13517 | 9466 | 13564 |
| 9420 | 13518 | 9467 | 13565 |
| 9421 | 13519 | 9468 | 13566 |
| 9422 | 13520 | 9469 | 13567 |
| 9423 | 13521 | 9470 | 13568 |
| 9424 | 13522 | 9471 | 13569 |
| 9425 | 13523 | 9472 | 13570 |
| 9426 | 13524 | 9473 | 13571 |
| 9427 | 13525 | 9474 | 13572 |
| 9428 | 13526 | 9475 | 13573 |
| 9429 | 13527 | 9476 | 13574 |
| 9430 | 13528 | 9477 | 13575 |
| 9431 | 13529 | 9478 | 13576 |
| 9432 | 13530 | 9479 | 13577 |
| 9433 | 13531 | 9480 | 13578 |
| 9434 | 13532 | 9481 | 13579 |
| 9435 | 13533 | 9482 | 13580 |
| 9436 | 13534 | 9483 | 13581 |
| 9437 | 13535 | 9484 | 13582 |
| 9438 | 13536 | 9485 | 13583 |
| 9439 | 13537 | 9486 | 13584 |
| 9440 | 13538 | 9487 | 13585 |
| 9441 | 13539 | 9488 | 13586 |
| 9442 | 13540 | 9489 | 13587 |
| 9443 | 13541 | 9490 | 13588 |
| 9444 | 13542 | 9491 | 13589 |
| 9445 | 13543 | 9492 | 13590 |
| 9446 | 13544 | 9493 | 13591 |
| 9447 | 13545 | 9494 | 13592 |
| 9448 | 13546 | 9495 | 13593 |
| 9449 | 13547 | 9496 | 13594 |
| 9450 | 13548 | 9497 | 13595 |
| 9451 | 13549 | 9498 | 13596 |
| 9452 | 13550 | 9499 | 13597 |
| 9453 | 13551 | 9500 | 13598 |
| 9454 | 13552 | 9501 | 13599 |
| 9455 | 13553 | 9502 | 13600 |
| 9503 | 13601 | 9550 | 13648 |
| 9504 | 13602 | 9551 | 13649 |
| 9505 | 13603 | 9552 | 13650 |
| 9506 | 13604 | 9553 | 13651 |
| 9507 | 13605 | 9554 | 13652 |
| 9508 | 13606 | 9555 | 13653 |
| 9509 | 13607 | 9556 | 13654 |
| 9510 | 13608 | 9557 | 13655 |
| 9511 | 13609 | 9558 | 13656 |
| 9512 | 13610 | 9559 | 13657 |
| 9513 | 13611 | 9560 | 13658 |
| 9514 | 13612 | 9561 | 13659 |
| 9515 | 13613 | 9562 | 13660 |
| 9516 | 13614 | 9563 | 13661 |
| 9517 | 13615 | 9564 | 13662 |
| 9518 | 13616 | 9565 | 13663 |
| 9519 | 13617 | 9566 | 13664 |
| 9520 | 13618 | 9567 | 13665 |
| 9521 | 13619 | 9568 | 13666 |
| 9522 | 13620 | 9569 | 13667 |
| 9523 | 13621 | 9570 | 13668 |
| 9524 | 13622 | 9571 | 13669 |
| 9525 | 13623 | 9572 | 13670 |
| 9526 | 13624 | 9573 | 13671 |
| 9527 | 13625 | 9574 | 13672 |
| 9528 | 13626 | 9575 | 13673 |
| 9529 | 13627 | 9576 | 13674 |
| 9530 | 13628 | 9577 | 13675 |
| 9531 | 13629 | 9578 | 13676 |
| 9532 | 13630 | 9579 | 13677 |
| 9533 | 13631 | 9580 | 13678 |
| 9534 | 13632 | 9581 | 13679 |
| 9535 | 13633 | 9582 | 13680 |
| 9536 | 13634 | 9583 | 13681 |
| 9537 | 13635 | 9584 | 13682 |
| 9538 | 13636 | 9585 | 13683 |
| 9539 | 13637 | 9586 | 13684 |
| 9540 | 13638 | 9587 | 13685 |
| 9541 | 13639 | 9588 | 13686 |
| 9542 | 13640 | 9589 | 13687 |
| 9543 | 13641 | 9590 | 13688 |
| 9544 | 13642 | 9591 | 13689 |
| 9545 | 13643 | 9592 | 13690 |
| 9546 | 13644 | 9593 | 13691 |
| 9547 | 13645 | 9594 | 13692 |
| 9548 | 13646 | 9595 | 13693 |
| 9549 | 13647 | 9596 | 13694 |
| 9597 | 13695 | 9644 | 13742 |
| 9598 | 13696 | 9645 | 13743 |
| 9599 | 13697 | 9646 | 13744 |
| 9600 | 13698 | 9647 | 13745 |
| 9601 | 13699 | 9648 | 13746 |
| 9602 | 13700 | 9649 | 13747 |
| 9603 | 13701 | 9650 | 13748 |
| 9604 | 13702 | 9651 | 13749 |
| 9605 | 13703 | 9652 | 13750 |
| 9606 | 13704 | 9653 | 13751 |
| 9607 | 13705 | 9654 | 13752 |
| 9608 | 13706 | 9655 | 13753 |
| 9609 | 13707 | 9656 | 13754 |
| 9610 | 13708 | 9657 | 13755 |
| 9611 | 13709 | 9658 | 13756 |
| 9612 | 13710 | 9659 | 13757 |
| 9613 | 13711 | 9660 | 13758 |
| 9614 | 13712 | 9661 | 13759 |
| 9615 | 13713 | 9662 | 13760 |
| 9616 | 13714 | 9663 | 13761 |
| 9617 | 13715 | 9664 | 13762 |
| 9618 | 13716 | 9665 | 13763 |
| 9619 | 13717 | 9666 | 13764 |
| 9620 | 13718 | 9667 | 13765 |
| 9621 | 13719 | 9668 | 13766 |
| 9622 | 13720 | 9669 | 13767 |
| 9623 | 13721 | 9670 | 13768 |
| 9624 | 13722 | 9671 | 13769 |
| 9625 | 13723 | 9672 | 13770 |
| 9626 | 13724 | 9673 | 13771 |
| 9627 | 13725 | 9674 | 13772 |
| 9628 | 13726 | 9675 | 13773 |
| 9629 | 13727 | 9676 | 13774 |
| 9630 | 13728 | 9677 | 13775 |
| 9631 | 13729 | 9678 | 13776 |
| 9632 | 13730 | 9679 | 13777 |
| 9633 | 13731 | 9680 | 13778 |
| 9634 | 13732 | 9681 | 13779 |
| 9635 | 13733 | 9682 | 13780 |
| 9636 | 13734 | 9683 | 13781 |
| 9637 | 13735 | 9684 | 13782 |
| 9638 | 13736 | 9685 | 13783 |
| 9639 | 13737 | 9686 | 13784 |
| 9640 | 13738 | 9687 | 13785 |
| 9641 | 13739 | 9688 | 13786 |
| 9642 | 13740 | 9689 | 13787 |
| 9643 | 13741 | 9690 | 13788 |
| 9691 | 13789 | 9738 | 13836 |
| 9692 | 13790 | 9739 | 13837 |
| 9693 | 13791 | 9740 | 13838 |
| 9694 | 13792 | 9741 | 13839 |
| 9695 | 13793 | 9742 | 13840 |
| 9696 | 13794 | 9743 | 13841 |
| 9697 | 13795 | 9744 | 13842 |
| 9698 | 13796 | 9745 | 13843 |
| 9699 | 13797 | 9746 | 13844 |
| 9700 | 13798 | 9747 | 13845 |
| 9701 | 13799 | 9748 | 13846 |
| 9702 | 13800 | 9749 | 13847 |
| 9703 | 13801 | 9750 | 13848 |
| 9704 | 13802 | 9751 | 13849 |
| 9705 | 13803 | 9752 | 13850 |
| 9706 | 13804 | 9753 | 13851 |
| 9707 | 13805 | 9754 | 13852 |
| 9708 | 13806 | 9755 | 13853 |
| 9709 | 13807 | 9756 | 13854 |
| 9710 | 13808 | 9757 | 13855 |
| 9711 | 13809 | 9758 | 13856 |
| 9712 | 13810 | 9759 | 13857 |
| 9713 | 13811 | 9760 | 13858 |
| 9714 | 13812 | 9761 | 13859 |
| 9715 | 13813 | 9762 | 13860 |
| 9716 | 13814 | 9763 | 13861 |
| 9717 | 13815 | 9764 | 13862 |
| 9718 | 13816 | 9765 | 13863 |
| 9719 | 13817 | 9766 | 13864 |
| 9720 | 13818 | 9767 | 13865 |
| 9721 | 13819 | 9768 | 13866 |
| 9722 | 13820 | 9769 | 13867 |
| 9723 | 13821 | 9770 | 13868 |
| 9724 | 13822 | 9771 | 13869 |
| 9725 | 13823 | 9772 | 13870 |
| 9726 | 13824 | 9773 | 13871 |
| 9727 | 13825 | 9774 | 13872 |
| 9728 | 13826 | 9775 | 13873 |
| 9729 | 13827 | 9776 | 13874 |
| 9730 | 13828 | 9777 | 13875 |
| 9731 | 13829 | 9778 | 13876 |
| 9732 | 13830 | 9779 | 13877 |
| 9733 | 13831 | 9780 | 13878 |
| 9734 | 13832 | 9781 | 13879 |
| 9735 | 13833 | 9782 | 13880 |
| 9736 | 13834 | 9783 | 13881 |
| 9737 | 13835 | 9784 | 13882 |
| 9785 | 13883 | 9832 | 13930 |
| 9786 | 13884 | 9833 | 13931 |
| 9787 | 13885 | 9834 | 13932 |
| 9788 | 13886 | 9835 | 13933 |
| 9789 | 13887 | 9836 | 13934 |
| 9790 | 13888 | 9837 | 13935 |
| 9791 | 13889 | 9838 | 13936 |
| 9792 | 13890 | 9839 | 13937 |
| 9793 | 13891 | 9840 | 13938 |
| 9794 | 13892 | 9841 | 13939 |
| 9795 | 13893 | 9842 | 13940 |
| 9796 | 13894 | 9843 | 13941 |
| 9797 | 13895 | 9844 | 13942 |
| 9798 | 13896 | 9845 | 13943 |
| 9799 | 13897 | 9846 | 13944 |
| 9800 | 13898 | 9847 | 13945 |
| 9801 | 13899 | 9848 | 13946 |
| 9802 | 13900 | 9849 | 13947 |
| 9803 | 13901 | 9850 | 13948 |
| 9804 | 13902 | 9851 | 13949 |
| 9805 | 13903 | 9852 | 13950 |
| 9806 | 13904 | 9853 | 13951 |
| 9807 | 13905 | 9854 | 13952 |
| 9808 | 13906 | 9855 | 13953 |
| 9809 | 13907 | 9856 | 13954 |
| 9810 | 13908 | 9857 | 13955 |
| 9811 | 13909 | 9858 | 13956 |
| 9812 | 13910 | 9859 | 13957 |
| 9813 | 13911 | 9860 | 13958 |
| 9814 | 13912 | 9861 | 13959 |
| 9815 | 13913 | 9862 | 13960 |
| 9816 | 13914 | 9863 | 13961 |
| 9817 | 13915 | 9864 | 13962 |
| 9818 | 13916 | 9865 | 13963 |
| 9819 | 13917 | 9866 | 13964 |
| 9820 | 13918 | 9867 | 13965 |
| 9821 | 13919 | 9868 | 13966 |
| 9822 | 13920 | 9869 | 13967 |
| 9823 | 13921 | 9870 | 13968 |
| 9824 | 13922 | 9871 | 13969 |
| 9825 | 13923 | 9872 | 13970 |
| 9826 | 13924 | 9873 | 13971 |
| 9827 | 13925 | 9874 | 13972 |
| 9828 | 13926 | 9875 | 13973 |
| 9829 | 13927 | 9876 | 13974 |
| 9830 | 13928 | 9877 | 13975 |
| 9831 | 13929 | 9878 | 13976 |
| 9879 | 13977 | 9926 | 14024 |
| 9880 | 13978 | 9927 | 14025 |
| 9881 | 13979 | 9928 | 14026 |
| 9882 | 13980 | 9929 | 14027 |
| 9883 | 13981 | 9930 | 14028 |
| 9884 | 13982 | 9931 | 14029 |
| 9885 | 13983 | 9932 | 14030 |
| 9886 | 13984 | 9933 | 14031 |
| 9887 | 13985 | 9934 | 14032 |
| 9888 | 13986 | 9935 | 14033 |
| 9889 | 13987 | 9936 | 14034 |
| 9890 | 13988 | 9937 | 14035 |
| 9891 | 13989 | 9938 | 14036 |
| 9892 | 13990 | 9939 | 14037 |
| 9893 | 13991 | 9940 | 14038 |
| 9894 | 13992 | 9941 | 14039 |
| 9895 | 13993 | 9942 | 14040 |
| 9896 | 13994 | 9943 | 14041 |
| 9897 | 13995 | 9944 | 14042 |
| 9898 | 13996 | 9945 | 14043 |
| 9899 | 13997 | 9946 | 14044 |
| 9900 | 13998 | 9947 | 14045 |
| 9901 | 13999 | 9948 | 14046 |
| 9902 | 14000 | 9949 | 14047 |
| 9903 | 14001 | 9950 | 14048 |
| 9904 | 14002 | 9951 | 14049 |
| 9905 | 14003 | 9952 | 14050 |
| 9906 | 14004 | 9953 | 14051 |
| 9907 | 14005 | 9954 | 14052 |
| 9908 | 14006 | 9955 | 14053 |
| 9909 | 14007 | 9956 | 14054 |
| 9910 | 14008 | 9957 | 14055 |
| 9911 | 14009 | 9958 | 14056 |
| 9912 | 14010 | 9959 | 14057 |
| 9913 | 14011 | 9960 | 14058 |
| 9914 | 14012 | 9961 | 14059 |
| 9915 | 14013 | 9962 | 14060 |
| 9916 | 14014 | 9963 | 14061 |
| 9917 | 14015 | 9964 | 14062 |
| 9918 | 14016 | 9965 | 14063 |
| 9919 | 14017 | 9966 | 14064 |
| 9920 | 14018 | 9967 | 14065 |
| 9921 | 14019 | 9968 | 14066 |
| 9922 | 14020 | 9969 | 14067 |
| 9923 | 14021 | 9970 | 14068 |
| 9924 | 14022 | 9971 | 14069 |
| 9925 | 14023 | 9972 | 14070 |
| 9973 | 14071 | | |
| 9974 | 14072 | | |
| 9975 | 14073 | | |
| 9976 | 14074 | | |
| 9977 | 14075 | | |
| 9978 | 14076 | | |
| 9979 | 14077 | | |
| 9980 | 14078 | | |
| 9981 | 14079 | | |
| 9982 | 14080 | | |
| 9983 | 14081 | | |
| 9984 | 14082 | | |
| 9985 | 14083 | | |
| 9986 | 14084 | | |
| 9987 | 14085 | | |
| 9988 | 14086 | | |
| 9989 | 14087 | | |
| 9990 | 14088 | | |
| 9991 | 14089 | | |
| 9992 | 14090 | | |
| 9993 | 14091 | | |
| 9994 | 14092 | | |
| 9995 | 14093 | | |
| 9996 | 14094 | | |
| 9997 | 14095 | | |
| 9998 | 14096 | | |
| 9999 | 14097 | | |
| 10000 | 14098 | | |
| 10001 | 14099 | | |
| 10002 | 14100 | | |
| 10003 | 14101 | | |
| 10004 | 14102 | | |
| 10005 | 14103 | | |
| 10006 | 14104 | | |
| 10007 | 14105 | | |
| 10008 | 14106 | | |
| 10009 | 14107 | | |
| 10010 | 14108 | | |
| 10011 | 14109 | | |
| 10012 | 14110 | | |

TABLE IA

| SeqID | Clone name | Organism |
|---|---|---|
| 8 | E3M10000001A02 | Enterococcus faecalis |
| 9 | E3M10000001A06 | Enterococcus faecalis |
| 10 | E3M10000001B01 | Enterococcus faecalis |
| 11 | E3M10000001B02 | Enterococcus faecalis |
| 12 | E3M10000001B05 | Enterococcus faecalis |
| 13 | E3M10000001B06 | Enterococcus faecalis |
| 14 | E3M10000001B08 | Enterococcus faecalis |
| 15 | E3M10000001B10 | Enterococcus faecalis |
| 16 | E3M10000001C02 | Enterococcus faecalis |
| 17 | E3M10000001C09 | Enterococcus faecalis |
| 18 | E3M10000001D02 | Enterococcus faecalis |
| 19 | E3M10000001D04 | Enterococcus faecalis |
| 20 | E3M10000001D05 | Enterococcus faecalis |
| 21 | E3M10000001D09 | Enterococcus faecalis |
| 22 | E3M10000001E01 | Enterococcus faecalis |
| 23 | E3M10000001E02 | Enterococcus faecalis |
| 24 | E3M10000001E03 | Enterococcus faecalis |
| 25 | E3M10000001E04 | Enterococcus faecalis |
| 26 | E3M10000001E08 | Enterococcus faecalis |
| 27 | E3M10000001E09 | Enterococcus faecalis |
| 28 | E3M10000001F02 | Enterococcus faecalis |
| 29 | E3M10000001F04 | Enterococcus faecalis |
| 30 | E3M10000001F06 | Enterococcus faecalis |
| 31 | E3M10000001F07 | Enterococcus faecalis |
| 32 | E3M10000001G02 | Enterococcus faecalis |
| 33 | E3M10000001G03 | Enterococcus faecalis |
| 34 | E3M10000001G04 | Enterococcus faecalis |
| 35 | E3M10000001G05 | Enterococcus faecalis |
| 36 | E3M10000001H02 | Enterococcus faecalis |
| 37 | E3M10000001H03 | Enterococcus faecalis |
| 38 | E3M10000001H04 | Enterococcus faecalis |
| 39 | E3M10000004A04 | Enterococcus faecalis |
| 40 | E3M10000004C03 | Enterococcus faecalis |
| 41 | E3M10000004D01 | Enterococcus faecalis |
| 42 | E3M10000004D02 | Enterococcus faecalis |
| 43 | E3M10000004D10 | Enterococcus faecalis |
| 44 | E3M10000004E11 | Enterococcus faecalis |
| 45 | E3M10000004F08 | Enterococcus faecalis |
| 46 | E3M10000004F10 | Enterococcus faecalis |
| 47 | E3M10000004G01 | Enterococcus faecalis |
| 48 | E3M10000004H11 | Enterococcus faecalis |
| 49 | E3M10000005A07 | Enterococcus faecalis |
| 50 | E3M10000005B01 | Enterococcus faecalis |
| 51 | E3M10000005B08 | Enterococcus faecalis |
| 52 | E3M10000005C01 | Enterococcus faecalis |
| 53 | E3M10000005C03 | Enterococcus faecalis |
| 54 | E3M10000005C04 | Enterococcus faecalis |
| 55 | E3M10000005D03 | Enterococcus faecalis |
| 56 | E3M10000005D04 | Enterococcus faecalis |
| 57 | E3M10000005D10 | Enterococcus faecalis |
| 58 | E3M10000005E01 | Enterococcus faecalis |
| 59 | E3M10000005E02 | Enterococcus faecalis |
| 60 | E3M10000005E03 | Enterococcus faecalis |
| 61 | E3M10000005E08 | Enterococcus faecalis |
| 62 | E3M10000005F07 | Enterococcus faecalis |
| 63 | E3M10000005F10 | Enterococcus faecalis |
| 64 | E3M10000005G05 | Enterococcus faecalis |
| 65 | E3M10000005H04 | Enterococcus faecalis |
| 66 | E3M10000006B03 | Enterococcus faecalis |
| 67 | E3M10000006C01 | Enterococcus faecalis |
| 68 | E3M10000006C12 | Enterococcus faecalis |
| 69 | E3M10000006D03 | Enterococcus faecalis |
| 70 | E3M10000006E11 | Enterococcus faecalis |
| 71 | E3M10000006F04 | Enterococcus faecalis |
| 72 | E3M10000006G04 | Enterococcus faecalis |
| 73 | E3M10000006G12 | Enterococcus faecalis |
| 74 | E3M10000006H09 | Enterococcus faecalis |
| 75 | E3M10000007A02 | Enterococcus faecalis |
| 76 | E3M10000007B02 | Enterococcus faecalis |
| 77 | E3M10000007B03 | Enterococcus faecalis |
| 78 | E3M10000007C03 | Enterococcus faecalis |
| 79 | E3M10000007C04 | Enterococcus faecalis |
| 80 | E3M10000007D03 | Enterococcus faecalis |
| 81 | E3M10000007E05 | Enterococcus faecalis |
| 82 | E3M10000007F01 | Enterococcus faecalis |
| 83 | E3M10000007F06 | Enterococcus faecalis |
| 84 | E3M10000007G01 | Enterococcus faecalis |
| 85 | E3M10000008C03 | Enterococcus faecalis |
| 86 | E3M10000008C08 | Enterococcus faecalis |
| 87 | E3M10000008C09 | Enterococcus faecalis |
| 88 | E3M10000008D08 | Enterococcus faecalis |
| 89 | E3M10000008E02 | Enterococcus faecalis |
| 90 | E3M10000008G05 | Enterococcus faecalis |
| 91 | E3M10000008G09 | Enterococcus faecalis |
| 92 | E3M10000008H02 | Enterococcus faecalis |
| 93 | E3M10000009C07 | Enterococcus faecalis |
| 94 | E3M10000009C09 | Enterococcus faecalis |
| 95 | E3M10000009D01 | Enterococcus faecalis |
| 96 | E3M10000009E02 | Enterococcus faecalis |
| 97 | E3M10000009E03 | Enterococcus faecalis |
| 98 | E3M10000009E05 | Enterococcus faecalis |
| 99 | E3M10000009G02 | Enterococcus faecalis |
| 100 | E3M10000010C08 | Enterococcus faecalis |
| 101 | E3M10000010D05 | Enterococcus faecalis |
| 102 | E3M10000010F01 | Enterococcus faecalis |
| 103 | E3M10000010G05 | Enterococcus faecalis |
| 104 | E3M10000010G07 | Enterococcus faecalis |
| 105 | E3M10000010G09 | Enterococcus faecalis |
| 106 | E3M10000010G10 | Enterococcus faecalis |
| 107 | E3M10000010H02 | Enterococcus faecalis |
| 108 | E3M10000011A09 | Enterococcus faecalis |
| 109 | E3M10000011B03 | Enterococcus faecalis |
| 110 | E3M10000011B09 | Enterococcus faecalis |
| 111 | E3M10000011C07 | Enterococcus faecalis |
| 112 | E3M10000011D03 | Enterococcus faecalis |
| 113 | E3M10000011H02 | Enterococcus faecalis |
| 114 | E3M10000011H05 | Enterococcus faecalis |
| 115 | E3M10000012B01 | Enterococcus faecalis |
| 116 | E3M10000012B02 | Enterococcus faecalis |
| 117 | E3M10000012B07 | Enterococcus faecalis |
| 118 | E3M10000012B08 | Enterococcus faecalis |
| 119 | E3M10000012C01 | Enterococcus faecalis |
| 120 | E3M10000012D10 | Enterococcus faecalis |
| 121 | E3M10000012E08 | Enterococcus faecalis |
| 122 | E3M10000012F05 | Enterococcus faecalis |
| 123 | E3M10000012F06 | Enterococcus faecalis |
| 124 | E3M10000012F07 | Enterococcus faecalis |
| 125 | E3M10000012F10 | Enterococcus faecalis |
| 126 | E3M10000012G02 | Enterococcus faecalis |
| 127 | E3M10000012G07 | Enterococcus faecalis |
| 128 | E3M10000013A06 | Enterococcus faecalis |
| 129 | E3M10000013A07 | Enterococcus faecalis |
| 130 | E3M10000013C05 | Enterococcus faecalis |
| 131 | E3M10000013D02 | Enterococcus faecalis |
| 132 | E3M10000013D08 | Enterococcus faecalis |
| 133 | E3M10000013D10 | Enterococcus faecalis |
| 134 | E3M10000013E02 | Enterococcus faecalis |
| 135 | E3M10000013E08 | Enterococcus faecalis |
| 136 | E3M10000013F05 | Enterococcus faecalis |
| 137 | E3M10000013F12 | Enterococcus faecalis |
| 138 | E3M10000013G10 | Enterococcus faecalis |
| 139 | E3M10000013H03 | Enterococcus faecalis |
| 140 | E3M10000013H05 | Enterococcus faecalis |
| 141 | E3M10000013H10 | Enterococcus faecalis |
| 142 | E3M10000014B12 | Enterococcus faecalis |
| 143 | E3M10000014E12 | Enterococcus faecalis |
| 144 | E3M10000014G09 | Enterococcus faecalis |
| 145 | E3M10000015B04 | Enterococcus faecalis |
| 146 | E3M10000015B12 | Enterococcus faecalis |
| 147 | E3M10000015E12 | Enterococcus faecalis |
| 148 | E3M10000016A03 | Enterococcus faecalis |
| 149 | E3M10000016A04 | Enterococcus faecalis |
| 150 | E3M10000016C11 | Enterococcus faecalis |
| 151 | E3M10000016D03 | Enterococcus faecalis |
| 152 | E3M10000016F06 | Enterococcus faecalis |
| 153 | E3M10000016F10 | Enterococcus faecalis |
| 154 | E3M10000016H05 | Enterococcus faecalis |
| 155 | E3M10000016H10 | Enterococcus faecalis |
| 156 | E3M10000017A09 | Enterococcus faecalis |
| 157 | E3M10000017D09 | Enterococcus faecalis |
| 158 | E3M10000018A07 | Enterococcus faecalis |
| 159 | E3M10000018C02 | Enterococcus faecalis |
| 160 | E3M10000018E01 | Enterococcus faecalis |
| 161 | E3M10000018G09 | Enterococcus faecalis |
| 162 | E3M10000018H06 | Enterococcus faecalis |
| 163 | E3M10000019B06 | Enterococcus faecalis |
| 164 | E3M10000019D02 | Enterococcus faecalis |
| 165 | E3M10000019E03 | Enterococcus faecalis |
| 166 | E3M10000019E04 | Enterococcus faecalis |
| 167 | E3M10000020G04 | Enterococcus faecalis |
| 168 | E3M10000020H05 | Enterococcus faecalis |
| 169 | E3M10000021A08 | Enterococcus faecalis |
| 170 | E3M10000021A11 | Enterococcus faecalis |
| 171 | E3M10000021B10 | Enterococcus faecalis |
| 172 | E3M10000021C03 | Enterococcus faecalis |
| 173 | E3M10000021C04 | Enterococcus faecalis |
| 174 | E3M10000021C08 | Enterococcus faecalis |
| 175 | E3M10000021D04 | Enterococcus faecalis |
| 176 | E3M10000021E10 | Enterococcus faecalis |
| 177 | E3M10000021G04 | Enterococcus faecalis |
| 178 | E3M10000021G10 | Enterococcus faecalis |
| 179 | E3M10000021G11 | Enterococcus faecalis |
| 180 | E3M10000021H11 | Enterococcus faecalis |
| 181 | E3M10000022A04 | Enterococcus faecalis |
| 182 | E3M10000022A11 | Enterococcus faecalis |
| 183 | E3M10000022B04 | Enterococcus faecalis |
| 184 | E3M10000022B05 | Enterococcus faecalis |
| 185 | E3M10000022B07 | Enterococcus faecalis |
| 186 | E3M10000022C05 | Enterococcus faecalis |
| 187 | E3M10000022C06 | Enterococcus faecalis |
| 188 | E3M10000022C09 | Enterococcus faecalis |
| 189 | E3M10000022D04 | Enterococcus faecalis |
| 190 | E3M10000022F05 | Enterococcus faecalis |
| 191 | E3M10000022F06 | Enterococcus faecalis |
| 192 | E3M10000022F08 | Enterococcus faecalis |
| 193 | E3M10000022G02 | Enterococcus faecalis |
| 194 | E3M10000022G12 | Enterococcus faecalis |
| 195 | E3M10000023A03 | Enterococcus faecalis |
| 196 | E3M10000023A06 | Enterococcus faecalis |
| 197 | E3M10000023A07 | Enterococcus faecalis |
| 198 | E3M10000023A09 | Enterococcus faecalis |
| 199 | E3M10000023B02 | Enterococcus faecalis |
| 200 | E3M10000023B06 | Enterococcus faecalis |
| 201 | E3M10000023C03 | Enterococcus faecalis |
| 202 | E3M10000023C04 | Enterococcus faecalis |
| 203 | E3M10000023C06 | Enterococcus faecalis |
| 204 | E3M10000023C08 | Enterococcus faecalis |
| 205 | E3M10000023C09 | Enterococcus faecalis |
| 206 | E3M10000023D02 | Enterococcus faecalis |
| 207 | E3M10000023D04 | Enterococcus faecalis |
| 208 | E3M10000023D10 | Enterococcus faecalis |
| 209 | E3M10000023E04 | Enterococcus faecalis |
| 210 | E3M10000023E07 | Enterococcus faecalis |
| 211 | E3M10000023E09 | Enterococcus faecalis |
| 212 | E3M10000023F02 | Enterococcus faecalis |
| 213 | E3M10000023F10 | Enterococcus faecalis |
| 214 | E3M10000023G02 | Enterococcus faecalis |
| 215 | E3M10000023G04 | Enterococcus faecalis |
| 216 | E3M10000023G10 | Enterococcus faecalis |
| 217 | E3M10000023H08 | Enterococcus faecalis |
| 218 | E3M10000024A03 | Enterococcus faecalis |
| 219 | E3M10000024A04 | Enterococcus faecalis |
| 220 | E3M10000024A08 | Enterococcus faecalis |
| 221 | E3M10000024C06 | Enterococcus faecalis |
| 222 | E3M10000025A06 | Enterococcus faecalis |
| 223 | E3M10000025B01 | Enterococcus faecalis |
| 224 | E3M10000025B03 | Enterococcus faecalis |
| 225 | E3M10000025B05 | Enterococcus faecalis |
| 226 | E3M10000025B10 | Enterococcus faecalis |
| 227 | E3M10000025C01 | Enterococcus faecalis |
| 228 | E3M10000025C04 | Enterococcus faecalis |
| 229 | E3M10000025C05 | Enterococcus faecalis |
| 230 | E3M10000025C07 | Enterococcus faecalis |
| 231 | E3M10000025C08 | Enterococcus faecalis |
| 232 | E3M10000025C09 | Enterococcus faecalis |
| 233 | E3M10000025C11 | Enterococcus faecalis |
| 234 | E3M10000025D0I | Enterococcus faecalis |
| 235 | E3M10000025D10 | Enterococcus faecalis |
| 236 | E3M10000025E07 | Enterococcus faecalis |
| 237 | E3M10000025E08 | Enterococcus faecalis |
| 238 | E3M10000025E12 | Enterococcus faecalis |
| 239 | E3M10000025F04 | Enterococcus faecalis |
| 240 | E3M10000025F06 | Enterococcus faecalis |
| 241 | E3M10000025F08 | Enterococcus faecalis |
| 242 | E3M10000025F09 | Enterococcus faecalis |
| 243 | E3M10000025F10 | Enterococcus faecalis |
| 244 | E3M10000025F11 | Enterococcus faecalis |
| 245 | E3M10000025F12 | Enterococcus faecalis |
| 246 | E3M10000025G02 | Enterococcus faecalis |
| 247 | E3M10000025G07 | Enterococcus faecalis |
| 248 | E3M10000025G09 | Enterococcus faecalis |
| 249 | E3M10000027A02 | Enterococcus faecalis |
| 250 | F3M10000027A07 | Enterococcus faecalis |
| 251 | E3M10000027A09 | Enterococcus faecalis |
| 252 | E3M10000027B07 | Enterococcus faecalis |
| 253 | E3M10000027B08 | Enterococcus faecalis |
| 254 | E3M10000027B09 | Enterococcus faecalis |
| 255 | E3M10000027C02 | Enterococcus faecalis |
| 256 | E3M10000027C03 | Enterococcus faecalis |
| 257 | E3M10000027C08 | Enterococcus faecalis |
| 258 | E3M10000027D03 | Enterococcus faecalis |
| 259 | E3M10000027D05 | Enterococcus faecalis |
| 260 | E3M10000027D08 | Enterococcus faecalis |
| 261 | E3M10000027D10 | Enterococcus faecalis |
| 262 | E3M10000027G01 | Enterococcus faecalis |
| 263 | E3M10000027G08 | Enterococcus faecalis |
| 264 | E3M10000027H04 | Enterococcus faecalis |
| 265 | E3M10000027H07 | Enterococcus faecalis |
| 266 | E3M10000028A02 | Enterococcus faecalis |
| 267 | E3M10000028A03 | Enterococcus faecalis |
| 268 | E3M10000028A04 | Enterococcus faecalis |
| 269 | E3M10000028A05 | Enterococcus faecalis |
| 270 | E3M10000028A06 | Enterococcus faecalis |
| 271 | E3M10000028A08 | Enterococcus faecalis |
| 272 | E3M10000028B01 | Enterococcus faecalis |
| 273 | E3M10000028B02 | Enterococcus faecalis |
| 274 | E3M10000028B03 | Enterococcus faecalis |
| 275 | E3M10000028B04 | Enterococcus faecalis |
| 276 | E3M10000028B05 | Enterococcus faecalis |
| 277 | E3M10000028B06 | Enterococcus faecalis |
| 278 | E3M10000028B07 | Enterococcus faecalis |
| 279 | E3M10000028B08 | Enterococcus faecalis |
| 280 | E3M10000028C01 | Enterococcus faecalis |
| 281 | E3M10000028C02 | Enterococcus faecalis |
| 282 | E3M10000028C04 | Enterococcus faecalis |
| 283 | E3M10000028C05 | Enterococcus faecalis |
| 284 | E3M10000028C06 | Enterococcus faecalis |
| 285 | E3M10000028C07 | Enterococcus faecalis |
| 286 | E3M10000028C08 | Enterococcus faecalis |
| 287 | E3M10000028D01 | Enterococcus faecalis |
| 288 | E3M10000028D02 | Enterococcus faecalis |
| 289 | E3M10000028D05 | Enterococcus faecalis |
| 290 | E3M10000028D06 | Enterococcus faecalis |
| 291 | E3M10000028D08 | Enterococcus faecalis |
| 292 | E3M10000028E01 | Enterococcus faecalis |
| 293 | E3M10000028E04 | Enterococcus faecalis |
| 294 | E3M10000028E07 | Enterococcus faecalis |
| 295 | E3M10000028F02 | Enterococcus faecalis |
| 296 | E3M10000028F03 | Enterococcus faecalis |
| 297 | E3M10000028F04 | Enterococcus faecalis |
| 298 | E3M10000028F05 | Enterococcus faecalis |
| 299 | E3M10000028F06 | Enterococcus faecalis |
| 300 | E3M10000028F07 | Enterococcus faecalis |
| 301 | E3M10000028G05 | Enterococcus faecalis |
| 302 | E3M10000028G06 | Enterococcus faecalis |
| 303 | E3M10000028G07 | Enterococcus faecalis |
| 304 | E3M10000028H04 | Enterococcus faecalis |
| 305 | E3M10000028H07 | Enterococcus faecalis |
| 306 | E3M10000029A02 | Enterococcus faecalis |
| 307 | E3M10000029A04 | Enterococcus faecalis |
| 308 | E3M10000029A05 | Enterococcus faecalis |
| 309 | E3M10000029A10 | Enterococcus faecalis |
| 310 | E3M10000029A11 | Enterococcus faecalis |
| 311 | E3M10000029B01 | Enterococcus faecalis |
| 312 | E3M10000029B02 | Enterococcus faecalis |
| 313 | E3M10000029B05 | Enterococcus faecalis |
| 314 | E3M10000029B06 | Enterococcus faecalis |
| 315 | E3M10000029B08 | Enterococcus faecalis |
| 316 | E3M10000029B11 | Enterococcus faecalis |
| 317 | E3M10000029B12 | Enterococcus faecalis |
| 318 | E3M10000029C01 | Enterococcus faecalis |
| 319 | E3M10000029C02 | Enterococcus faecalis |
| 320 | E3M10000029C03 | Enterococcus faecalis |
| 321 | E3M10000029C04 | Enterococcus faecalis |
| 322 | E3M10000029C05 | Enterococcus faecalis |
| 323 | E3M10000029C06 | Enterococcus faecalis |
| 324 | E3M10000029C07 | Enterococcus faecalis |
| 325 | E3M10000029C08 | Enterococcus faecalis |
| 326 | E3M10000029C09 | Enterococcus faecalis |
| 327 | E3M10000029C10 | Enterococcus faecalis |
| 328 | E3M10000029C12 | Enterococcus faecalis |
| 329 | E3M10000029D01 | Enterococcus faecalis |
| 330 | E3M10000029D03 | Enterococcus faecalis |
| 331 | E3M10000029D04 | Enterococcus faecalis |
| 332 | E3M10000029D05 | Enterococcus faecalis |
| 333 | E3M10000029D06 | Enterococcus faecalis |
| 334 | E3M10000029D08 | Enterococcus faecalis |
| 335 | E3M10000029D12 | Enterococcus faecalis |
| 336 | E3M10000029E01 | Enterococcus faecalis |
| 337 | E3M10000029E02 | Enterococcus faecalis |
| 338 | E3M10000029E03 | Enterococcus faecalis |
| 339 | E3M10000029E05 | Enterococcus faecalis |
| 340 | E3M10000029E07 | Enterococcus faecalis |
| 341 | E3M10000029E08 | Enterococcus faecalis |
| 342 | E3M10000029E09 | Enterococcus faecalis |
| 343 | E3M10000029E12 | Enterococcus faecalis |
| 344 | E3M10000029F01 | Enterococcus faecalis |
| 345 | E3M10000029F05 | Enterococcus faecalis |
| 346 | E3M10000029F06 | Enterococcus faecalis |
| 347 | E3M10000029F09 | Enterococcus faecalis |
| 348 | E3M10000029F10 | Enterococcus faecalis |
| 349 | E3M10000029F11 | Enterococcus faecalis |
| 350 | E3M10000029F12 | Enterococcus faecalis |
| 351 | E3M10000029G01 | Enterococcus faecalis |
| 352 | E3M10000029G04 | Enterococcus faecalis |
| 353 | E3M10000029G05 | Enterococcus faecalis |
| 354 | E3M10000029G07 | Enterococcus faecalis |
| 355 | E3M10000029G08 | Enterococcus faecalis |
| 356 | E3M10000029G09 | Enterococcus faecalis |
| 357 | E3M10000029G10 | Enterococcus faecalis |
| 358 | E3M10000029G11 | Enterococcus faecalis |
| 359 | E3M10000029G12 | Enterococcus faecalis |
| 360 | E3M10000029H02 | Enterococcus faecalis |
| 361 | E3M10000029H04 | Enterococcus faecalis |
| 362 | E3M10000029H05 | Enterococcus faecalis |
| 363 | E3M10000029H07 | Enterococcus faecalis |
| 364 | E3M10000029H08 | Enterococcus faecalis |
| 365 | E3M10000029H11 | Enterococcus faecalis |
| 366 | E3M10000030A05 | Enterococcus faecalis |
| 367 | E3M10000030A08 | Enterococcus faecalis |
| 368 | E3M10000030A09 | Enterococcus faecalis |
| 369 | E3M10000030A11 | Enterococcus faecalis |
| 370 | E3M10000030B03 | Enterococcus faecalis |
| 371 | E3M10000030B04 | Enterococcus faecalis |
| 372 | E3M10000030B05 | Enterococcus faecalis |
| 373 | E3M10000030B06 | Enterococcus faecalis |
| 374 | E3M10000030B07 | Enterococcus faecalis |
| 375 | E3M10000030B08 | Enterococcus faecalis |
| 376 | E3M10000030B10 | Enterococcus faecalis |
| 377 | E3M10000030B11 | Enterococcus faecalis |
| 378 | E3M10000030B12 | Enterococcus faecalis |
| 379 | E3M10000030C03 | Enterococcus faecalis |
| 380 | E3M10000030C04 | Enterococcus faecalis |
| 381 | E3M10000030C12 | Enterococcus faecalis |
| 382 | E3M10000030D02 | Enterococcus faecalis |
| 383 | E3M10000030D05 | Enterococcus faecalis |
| 384 | E3M10000030D08 | Enterococcus faecalis |
| 385 | E3M10000030D09 | Enterococcus faecalis |
| 386 | E3M10000030D10 | Enterococcus faecalis |
| 387 | E3M10000030D12 | Enterococcus faecalis |
| 388 | E3M10000030E01 | Enterococcus faecalis |
| 389 | E3M10000030E02 | Enterococcus faecalis |
| 390 | E3M10000030E04 | Enterococcus faecalis |
| 391 | E3M10000030E08 | Enterococcus faecalis |
| 392 | E3M10000030E09 | Enterococcus faecalis |
| 393 | E3M10000030E10 | Enterococcus faecalis |
| 394 | E3M10000030F01 | Enterococcus faecalis |
| 395 | E3M10000030F04 | Enterococcus faecalis |
| 396 | E3M10000030F06 | Enterococcus faecalis |
| 397 | E3M10000030F07 | Enterococcus faecalis |
| 398 | E3M10000030F10 | Enterococcus faecalis |
| 399 | E3M10000030F12 | Enterococcus faecalis |
| 400 | E3M10000030G01 | Enterococcus faecalis |
| 401 | E3M10000030G03 | Enterococcus faecalis |
| 402 | E3M10000030G06 | Enterococcus faecalis |
| 403 | E3M10000030G08 | Enterococcus faecalis |
| 404 | E3M10000030G09 | Enterococcus faecalis |
| 405 | E3M10000030G12 | Enterococcus faecalis |
| 406 | E3M10000030H03 | Enterococcus faecalis |
| 407 | E3M10000030H04 | Enterococcus faecalis |
| 408 | E3M10000030H06 | Enterococcus faecalis |
| 409 | E3M10000030H07 | Enterococcus faecalis |
| 410 | E3M10000030H08 | Enterococcus faecalis |
| 411 | E3M10000030H10 | Enterococcus faecalis |
| 412 | E3M10000030H11 | Enterococcus faecalis |
| 413 | E3M10000031A02 | Enterococcus faecalis |
| 414 | E3M10000031A06 | Enterococcus faecalis |
| 415 | E3M10000031A07 | Enterococcus faecalis |
| 416 | E3M10000031A08 | Enterococcus faecalis |
| 417 | E3M10000031B02 | Enterococcus faecalis |
| 418 | E3M10000031B03 | Enterococcus faecalis |
| 419 | E3M10000031B04 | Enterococcus faecalis |
| 420 | E3M10000031B09 | Enterococcus faecalis |
| 421 | E3M10000031B10 | Enterococcus faecalis |
| 422 | E3M10000031B11 | Enterococcus faecalis |
| 423 | E3M10000031B12 | Enterococcus faecalis |
| 424 | E3M10000031C01 | Enterococcus faecalis |
| 425 | E3M10000031C04 | Enterococcus faecalis |
| 426 | E3M10000031C06 | Enterococcus faecalis |
| 427 | E3M10000031C10 | Enterococcus faecalis |
| 428 | E3M10000031C11 | Enterococcus faecalis |
| 429 | E3M10000031C12 | Enterococcus faecalis |
| 430 | E3M10000031D03 | Enterococcus faecalis |
| 431 | E3M10000031D04 | Enterococcus faecalis |
| 432 | E3M10000031D08 | Enterococcus faecalis |
| 433 | E3M10000031E03 | Enterococcus faecalis |
| 434 | E3M10000031E09 | Enterococcus faecalis |
| 435 | E3M10000031F02 | Enterococcus faecalis |
| 436 | E3M10000031F04 | Enterococcus faecalis |
| 437 | E3M10000031F07 | Enterococcus faecalis |
| 438 | E3M10000031F09 | Enterococcus faecalis |
| 439 | E3M10000031F11 | Enterococcus faecalis |
| 440 | E3M10000031G03 | Enterococcus faecalis |
| 441 | E3M10000031G04 | Enterococcus faecalis |
| 442 | E3M10000031G05 | Enterococcus faecalis |
| 443 | E3M10000031G06 | Enterococcus faecalis |
| 444 | E3M10000031G07 | Enterococcus faecalis |
| 445 | E3M10000031G08 | Enterococcus faecalis |
| 446 | E3M10000031G11 | Enterococcus faecalis |
| 447 | E3M10000031H05 | Enterococcus faecalis |
| 448 | E3M10000031H06 | Enterococcus faecalis |
| 449 | E3M10000031H07 | Enterococcus faecalis |
| 450 | E3M10000031H08 | Enterococcus faecalis |
| 451 | E3M10000031H10 | Enterococcus faecalis |
| 452 | E3M10000031H11 | Enterococcus faecalis |
| 453 | E3M10000032A02 | Enterococcus faecalis |
| 454 | E3M10000032A04 | Enterococcus faecalis |
| 455 | E3M10000032A06 | Enterococcus faecalis |
| 456 | E3M10000032A07 | Enterococcus faecalis |
| 457 | E3M10000032A08 | Enterococcus faecalis |
| 458 | E3M10000032A09 | Enterococcus faecalis |
| 459 | E3M10000032A10 | Enterococcus faecalis |
| 460 | E3M10000032A11 | Enterococcus faecalis |
| 461 | E3M10000032B03 | Enterococcus faecalis |
| 462 | E3M10000032B04 | Enterococcus faecalis |
| 463 | E3M10000032B07 | Enterococcus faecalis |
| 464 | E3M10000032B08 | Enterococcus faecalis |
| 465 | E3M10000032B09 | Enterococcus faecalis |
| 466 | E3M10000032B11 | Enterococcus faecalis |
| 467 | E3M10000032B12 | Enterococcus faecalis |
| 468 | E3M10000032C01 | Enterococcus faecalis |
| 469 | E3M10000032C02 | Enterococcus faecalis |
| 470 | E3M10000032C03 | Enterococcus faecalis |
| 471 | E3M10000032C04 | Enterococcus faecalis |
| 472 | E3M10000032C06 | Enterococcus faecalis |
| 473 | E3M10000032C09 | Enterococcus faecalis |
| 474 | E3M10000032C11 | Enterococcus faecalis |
| 475 | E3M10000032C12 | Enterococcus faecalis |
| 476 | E3M10000032D01 | Enterococcus faecalis |
| 477 | E3M10000032D02 | Enterococcus faecalis |
| 478 | E3M10000032D03 | Enterococcus faecalis |
| 479 | E3M10000032D06 | Enterococcus faecalis |
| 480 | E3M10000032D09 | Enterococcus faecalis |
| 481 | E3M10000032D12 | Enterococcus faecalis |
| 482 | E3M10000032E04 | Enterococcus faecalis |
| 483 | E3M10000032E05 | Enterococcus faecalis |
| 484 | E3M10000032E08 | Enterococcus faecalis |
| 485 | E3M10000032E10 | Enterococcus faecalis |
| 486 | E3M10000032E11 | Enterococcus faecalis |
| 487 | E3M10000032E12 | Enterococcus faecalis |
| 488 | E3M10000032F02 | Enterococcus faecalis |
| 489 | E3M10000032F03 | Enterococcus faecalis |
| 490 | E3M10000032F05 | Enterococcus faecalis |
| 491 | E3M10000032F07 | Enterococcus faecalis |
| 492 | E3M10000032F08 | Enterococcus faecalis |
| 493 | E3M10000032F11 | Enterococcus faecalis |
| 494 | E3M10000032F12 | Enterococcus faecalis |
| 495 | E3M10000032G01 | Enterococcus faecalis |
| 496 | E3M10000032G02 | Enterococcus faecalis |
| 497 | E3M10000032G04 | Enterococcus faecalis |
| 498 | E3M10000032G05 | Enterococcus faecalis |
| 499 | E3M10000032G06 | Enterococcus faecalis |
| 500 | E3M10000032G07 | Enterococcus faecalis |
| 501 | E3M10000032H05 | Enterococcus faecalis |
| 502 | E3M10000032H06 | Enterococcus faecalis |
| 503 | E3M10000032H08 | Enterococcus faecalis |
| 504 | E3M10000032H09 | Enterococcus faecalis |
| 505 | E3M10000032H10 | Enterococcus faecalis |
| 506 | E3M10000033A03 | Enterococcus faecalis |
| 507 | E3M10000033A04 | Enterococcus faecalis |
| 508 | E3M10000033A05 | Enterococcus faecalis |
| 509 | E3M10000033A06 | Enterococcus faecalis |
| 510 | E3M10000033A07 | Enterococcus faecalis |
| 511 | E3M10000033A08 | Enterococcus faecalis |
| 512 | E3M10000033A11 | Enterococcus faecalis |
| 513 | E3M10000033B01 | Enterococcus faecalis |
| 514 | E3M10000033B02 | Enterococcus faecalis |
| 515 | E3M10000033B04 | Enterococcus faecalis |
| 516 | E3M10000033B05 | Enterococcus faecalis |
| 517 | E3M10000033B06 | Enterococcus faecalis |
| 518 | E3M10000033B08 | Enterococcus faecalis |
| 519 | E3M10000033B09 | Enterococcus faecalis |
| 520 | E3M10000033C01 | Enterococcus faecalis |
| 521 | E3M10000033C02 | Enterococcus faecalis |
| 522 | E3M10000033C05 | Enterococcus faecalis |
| 523 | E3M10000033C09 | Enterococcus faecalis |
| 524 | E3M10000033C10 | Enterococcus faecalis |
| 525 | E3M10000033C11 | Enterococcus faecalis |
| 526 | E3M10000033C12 | Enterococcus faecalis |
| 527 | E3M10000033D01 | Enterococcus faecalis |
| 528 | E3M10000033D04 | Enterococcus faecalis |
| 529 | E3M10000033D05 | Enterococcus faecalis |
| 530 | E3M10000033D06 | Enterococcus faecalis |
| 531 | E3M10000033D09 | Enterococcus faecalis |
| 532 | E3M10000033D10 | Enterococcus faecalis |
| 533 | E3M10000033D11 | Enterococcus faecalis |
| 534 | E3M10000033E02 | Enterococcus faecalis |
| 535 | E3M10000033E03 | Enterococcus faecalis |
| 536 | E3M10000033E04 | Enterococcus faecalis |
| 537 | E3M10000033E05 | Enterococcus faecalis |
| 538 | E3M10000033E07 | Enterococcus faecalis |
| 539 | E3M10000033E08 | Enterococcus faecalis |
| 540 | E3M10000033E09 | Enterococcus faecalis |
| 541 | E3M10000033E11 | Enterococcus faecalis |
| 542 | E3M10000033F01 | Enterococcus faecalis |
| 543 | E3M10000033F03 | Enterococcus faecalis |
| 544 | E3M10000033F04 | Enterococcus faecalis |
| 545 | E3M10000033F05 | Enterococcus faecalis |
| 546 | E3M10000033F07 | Enterococcus faecalis |
| 547 | E3M10000033F08 | Enterococcus faecalis |
| 548 | E3M10000033F10 | Enterococcus faecalis |
| 549 | E3M10000033F12 | Enterococcus faecalis |
| 550 | E3M10000033G01 | Enterococcus faecalis |
| 551 | E3M10000033G02 | Enterococcus faecalis |
| 552 | E3M10000033G03 | Enterococcus faecalis |
| 553 | E3M10000033G04 | Enterococcus faecalis |
| 554 | E3M10000033G06 | Enterococcus faecalis |
| 555 | E3M10000033G07 | Enterococcus faecalis |
| 556 | E3M10000033G08 | Enterococcus faecalis |
| 557 | E3M10000033G09 | Enterococcus faecalis |
| 558 | E3M10000033G12 | Enterococcus faecalis |
| 559 | E3M10000033H02 | Enterococcus faecalis |
| 560 | E3M10000033H04 | Enterococcus faecalis |
| 561 | E3M10000033H05 | Enterococcus faecalis |
| 562 | E3M10000033H07 | Enterococcus faecalis |
| 563 | E3M10000033H08 | Enterococcus faecalis |
| 564 | E3M10000033H09 | Enterococcus faecalis |
| 565 | E3M10000033H10 | Enterococcus faecalis |
| 566 | E3M10000033H11 | Enterococcus faecalis |
| 567 | E3M10000034A02 | Enterococcus faecalis |
| 568 | E3M10000034A03 | Enterococcus faecalis |
| 569 | E3M10000034A04 | Enterococcus faecalis |
| 570 | E3M10000034B02 | Enterococcus faecalis |
| 571 | E3M10000034B04 | Enterococcus faecalis |
| 572 | E3M10000034C04 | Enterococcus faecalis |
| 573 | E3M10000034D01 | Enterococcus faecalis |
| 574 | E3M10000034D02 | Enterococcus faecalis |
| 575 | E3M10000034E01 | Enterococcus faecalis |
| 576 | E3M10000034E04 | Enterococcus faecalis |
| 577 | E3M10000034F02 | Enterococcus faecalis |
| 578 | E3M10000034F03 | Enterococcus faecalis |
| 579 | E3M10000034F04 | Enterococcus faecalis |
| 580 | E3M10000034G02 | Enterococcus faecalis |
| 581 | E3M10000034G03 | Enterococcus faecalis |
| 582 | E3M10000034H02 | Enterococcus faecalis |
| 583 | E3M10000034H03 | Enterococcus faecalis |
| 584 | E3M10000035A02 | Enterococcus faecalis |
| 585 | E3M10000035A04 | Enterococcus faecalis |
| 586 | E3M10000035A05 | Enterococcus faecalis |
| 587 | E3M10000035A06 | Enterococcus faecalis |
| 588 | E3M10000035A08 | Enterococcus faecalis |
| 589 | E3M10000035A09 | Enterococcus faecalis |
| 590 | E3M1000003SA11 | Enterococcus faecalis |
| 591 | E3M10000035B01 | Enterococcus faecalis |
| 592 | E3M10000035B03 | Enterococcus faecalis |
| 593 | E3M10000035B06 | Enterococcus faecalis |
| 594 | E3M10000035B07 | Enterococcus faecalis |
| 595 | E3M10000035B08 | Enterococcus faecalis |
| 596 | E3M10000035B10 | Enterococcus faecalis |
| 597 | E3M1000003SB11 | Enterococcus faecalis |
| 598 | E3M10000035B12 | Enterococcus faecalis |
| 599 | E3M10000035C01 | Enterococcus faecalis |
| 600 | E3M10000035C03 | Enterococcus faecalis |
| 601 | E3M10000035C04 | Enterococcus faecalis |
| 602 | E3M1000003SC05 | Enterococcus faecalis |
| 603 | E3M10000035C06 | Enterococcus faecalis |
| 604 | E3M10000035C07 | Enterococcus faecalis |
| 605 | E3M10000035C08 | Enterococcus faecalis |
| 606 | E3M10000035C09 | Enterococcus faecalis |
| 607 | E3M10000035C11 | Enterococcus faecalis |
| 608 | E3M10000035C12 | Enterococcus faecalis |
| 609 | E3M10000035D02 | Enterococcus faecalis |
| 610 | E3M10000035D03 | Enterococcus faecalis |
| 611 | E3M10000035D04 | Enterococcus faecalis |
| 612 | E3M10000035D05 | Enterococcus faecalis |
| 613 | E3M10000035D10 | Enterococcus faecalis |
| 614 | E3M10000035D11 | Enterococcus faecalis |
| 615 | E3M10000035E03 | Enterococcus faecalis |
| 616 | E3M10000035E04 | Enterococcus faecalis |
| 617 | E3M10000035E05 | Enterococcus faecalis |
| 618 | E3M10000035E07 | Enterococcus faecalis |
| 619 | E3M10000035E08 | Enterococcus faecalis |
| 620 | E3M10000035E09 | Enterococcus faecalis |
| 621 | E3M10000035E10 | Enterococcus faecalis |
| 622 | E3M10000035E11 | Enterococcus faecalis |
| 623 | E3M10000035E12 | Enterococcus faecalis |
| 624 | E3M10000035F01 | Enterococcus faecalis |
| 625 | E3M10000035F02 | Enterococcus faecalis |
| 626 | E3M10000035F03 | Enterococcus faecalis |
| 627 | E3M10000035F06 | Enterococcus faecalis |
| 628 | E3M10000035F07 | Enterococcus faecalis |
| 629 | E3M10000035F08 | Enterococcus faecalis |
| 630 | E3M10000035F09 | Enterococcus faecalis |
| 631 | E3M10000035F11 | Enterococcus faecalis |
| 632 | E3M10000035F12 | Enterococcus faecalis |
| 633 | E3M10000035G02 | Enterococcus faecalis |
| 634 | E3M10000035G04 | Enterococcus faecalis |
| 635 | E3M10000035G05 | Enterococcus faecalis |
| 636 | E3M10000035G08 | Enterococcus faecalis |
| 637 | E3M10000035G09 | Enterococcus faecalis |
| 638 | E3M10000035G10 | Enterococcus faecalis |
| 639 | E3M10000035G11 | Enterococcus faecalis |
| 640 | E3M10000035H03 | Enterococcus faecalis |
| 641 | E3M10000035H06 | Enterococcus faecalis |
| 642 | E3M10000035H09 | Enterococcus faecalis |
| 643 | E3M10000035H11 | Enterococcus faecalis |
| 644 | E3M10000036A03 | Enterococcus faecalis |
| 645 | E3M10000036A04 | Enterococcus faecalis |
| 646 | E3M10000036A05 | Enterococcus faecalis |
| 647 | E3M10000036A06 | Enterococcus faecalis |
| 648 | E3M10000036A07 | Enterococcus faecalis |
| 649 | E3M10000036A08 | Enterococcus faecalis |
| 650 | E3M10000036A09 | Enterococcus faecalis |
| 651 | E3M10000036A10 | Enterococcus faecalis |
| 652 | E3M10000036B01 | Enterococcus faecalis |
| 653 | E3M10000036B03 | Enterococcus faecalis |
| 654 | E3M10000036B06 | Enterococcus faecalis |
| 655 | E3M10000036B07 | Enterococcus faecalis |
| 656 | E3M10000036B08 | Enterococcus faecalis |
| 657 | E3M10000036B09 | Enterococcus faecalis |
| 658 | E3M10000036B11 | Enterococcus faecalis |
| 659 | E3M10000036B12 | Enterococcus faecalis |
| 660 | E3M10000036C01 | Enterococcus faecalis |
| 661 | E3M10000036C03 | Enterococcus faecalis |
| 662 | E3M10000036C06 | Enterococcus faecalis |
| 663 | E3M10000036C07 | Enterococcus faecalis |
| 664 | E3M10000036C08 | Enterococcus faecalis |
| 665 | E3M10000036C09 | Enterococcus faecalis |
| 666 | E3M10000036C10 | Enterococcus faecalis |
| 667 | E3M10000036C11 | Enterococcus faecalis |
| 668 | E3M10000036D03 | Enterococcus faecalis |
| 669 | E3M10000036D04 | Enterococcus faecalis |
| 670 | E3M10000036D06 | Enterococcus faecalis |
| 671 | E3M10000036D08 | Enterococcus faecalis |
| 672 | E3M10000036D09 | Enterococcus faecalis |
| 673 | E3M10000036D10 | Enterococcus faecalis |
| 674 | E3M10000036D11 | Enterococcus faecalis |
| 675 | E3M10000036D12 | Enterococcus faecalis |
| 676 | E3M10000036E01 | Enterococcus faecalis |
| 677 | E3M10000036E04 | Enterococcus faecalis |
| 678 | E3M10000036E05 | Enterococcus faecalis |
| 679 | E3M10000036E07 | Enterococcus faecalis |
| 680 | E3M10000036E08 | Enterococcus faecalis |
| 681 | E3M10000036F03 | Enterococcus faecalis |
| 682 | E3M10000036F04 | Enterococcus faecalis |
| 683 | E3M10000036F05 | Enterococcus faecalis |
| 684 | E3M10000036F08 | Enterococcus faecalis |
| 685 | E3M10000036F09 | Enterococcus faecalis |
| 686 | E3M10000036F10 | Enterococcus faecalis |
| 687 | E3M10000036F12 | Enterococcus faecalis |
| 688 | E3M10000036G01 | Enterococcus faecalis |
| 689 | E3M10000036G02 | Enterococcus faecalis |
| 690 | E3M10000036G03 | Enterococcus faecalis |
| 691 | E3M10000036G04 | Enterococcus faecalis |
| 692 | E3M10000036G06 | Enterococcus faecalis |
| 693 | E3M10000036G10 | Enterococcus faecalis |
| 694 | E3M10000036H02 | Enterococcus faecalis |
| 695 | E3M10000036H03 | Enterococcus faecalis |
| 696 | E3M10000036H04 | Enterococcus faecalis |
| 697 | E3M10000036H05 | Enterococcus faecalis |
| 698 | E3M10000036H06 | Enterococcus faecalis |
| 699 | E3M10000036H07 | Enterococcus faecalis |
| 700 | E3M10000036H08 | Enterococcus faecalis |
| 701 | E3M10000036H09 | Enterococcus faecalis |
| 702 | E3M10000036H10 | Enterococcus faecalis |
| 703 | E3M10000037A03 | Enterococcus faecalis |
| 704 | E3M10000037A06 | Enterococcus faecalis |
| 705 | E3M10000037A08 | Enterococcus faecalis |
| 706 | E3M10000037A09 | Enterococcus faecalis |
| 707 | E3M10000037A10 | Enterococcus faecalis |
| 708 | E3M10000037B02 | Enterococcus faecalis |
| 709 | E3M10000037B07 | Enterococcus faecalis |
| 710 | E3M10000037B08 | Enterococcus faecalis |
| 711 | E3M10000037B11 | Enterococcus faecalis |
| 712 | E3M10000037C01 | Enterococcus faecalis |
| 713 | E3M10000037C02 | Enterococcus faecalis |
| 714 | E3M10000037C04 | Enterococcus faecalis |
| 715 | E3M10000037C05 | Enterococcus faecalis |
| 716 | E3M10000037C07 | Enterococcus faecalis |
| 717 | E3M10000037C11 | Enterococcus faecalis |
| 718 | E3M10000037C12 | Enterococcus faecalis |
| 719 | E3M10000037D02 | Enterococcus faecalis |
| 720 | E3M10000037D03 | Enterococcus faecalis |
| 721 | E3M10000037D04 | Enterococcus faecalis |
| 722 | E3M10000037D05 | Enterococcus faecalis |
| 723 | E3M10000037D06 | Enterococcus faecalis |
| 724 | E3M10000037D09 | Enterococcus faecalis |
| 725 | E3M10000037D11 | Enterococcus faecalis |
| 726 | E3M10000037E01 | Enterococcus faecalis |
| 727 | E3M10000037E02 | Enterococcus faecalis |
| 728 | E3M10000037E03 | Enterococcus faecalis |
| 729 | E3M10000037E05 | Enterococcus faecalis |
| 730 | E3M10000037E07 | Enterococcus faecalis |
| 731 | E3M10000037E08 | Enterococcus faecalis |
| 732 | E3M10000037E10 | Enterococcus faecalis |
| 733 | E3M10000037E12 | Enterococcus faecalis |
| 734 | E3M10000037F01 | Enterococcus faecalis |
| 735 | E3M10000037F02 | Enterococcus faecalis |
| 736 | E3M10000037F06 | Enterococcus faecalis |
| 737 | E3M10000037F07 | Enterococcus faecalis |
| 738 | E3M10000037F12 | Enterococcus faecalis |
| 739 | E3M10000037G01 | Enterococcus faecalis |
| 740 | E3M10000037G02 | Enterococcus faecalis |
| 741 | E3M10000037G03 | Enterococcus faecalis |
| 742 | E3M10000037G05 | Enterococcus faecalis |
| 743 | E3M10000037G06 | Enterococcus faecalis |
| 744 | E3M10000037G07 | Enterococcus faecalis |
| 745 | E3M10000037G08 | Enterococcus faecalis |
| 746 | E3M10000037G10 | Enterococcus faecalis |
| 747 | E3M10000037G11 | Enterococcus faecalis |
| 748 | E3M10000037H02 | Enterococcus faecalis |
| 749 | E3M10000037H05 | Enterococcus faecalis |
| 750 | E3M10000037H07 | Enterococcus faecalis |
| 751 | E3M10000037H10 | Enterococcus faecalis |
| 752 | E3M10000037H11 | Enterococcus faecalis |
| 753 | E3M10000038A02 | Enterococcus faecalis |
| 754 | E3M10000038A03 | Enterococcus faecalis |
| 755 | E3M10000038A05 | Enterococcus faecalis |
| 756 | E3M10000038A06 | Enterococcus faecalis |
| 757 | E3M10000038A07 | Enterococcus faecalis |
| 758 | E3M10000038A09 | Enterococcus faecalis |
| 759 | E3M10000038A10 | Enterococcus faecalis |
| 760 | E3M10000038A11 | Enterococcus faecalis |
| 761 | E3M10000038B02 | Enterococcus faecalis |
| 762 | E3M10000038B03 | Enterococcus faecalis |
| 763 | E3M10000038B04 | Enterococcus faecalis |
| 764 | E3M10000038B05 | Enterococcus faecalis |
| 765 | E3M10000038B07 | Enterococcus faecalis |
| 766 | E3M10000038B08 | Enterococcus faecalis |
| 767 | E3M10000038B09 | Enterococcus faecalis |
| 768 | E3M10000038B11 | Enterococcus faecalis |
| 769 | E3M10000038C02 | Enterococcus faecalis |
| 770 | E3M10000038C03 | Enterococcus faecalis |
| 771 | E3M10000038C05 | Enterococcus faecalis |
| 772 | E3M10000038C07 | Enterococcus faecalis |
| 773 | E3M10000038C10 | Enterococcus faecalis |
| 774 | E3M10000038C12 | Enterococcus faecalis |
| 775 | E3M10000038D01 | Enterococcus faecalis |
| 776 | E3M10000038D02 | Enterococcus faecalis |
| 777 | E3M10000038D04 | Enterococcus faecalis |
| 778 | E3M10000038D08 | Enterococcus faecalis |
| 779 | E3M10000038D10 | Enterococcus faecalis |
| 780 | E3M10000038D11 | Enterococcus faecalis |
| 781 | E3M10000038D12 | Enterococcus faecalis |
| 782 | E3M10000038E02 | Enterococcus faecalis |
| 783 | E3M10000038E03 | Enterococcus faecalis |
| 784 | E3M10000038E04 | Enterococcus faecalis |
| 785 | E3M10000038E05 | Enterococcus faecalis |
| 786 | E3M10000038E07 | Enterococcus faecalis |
| 787 | E3M10000038E08 | Enterococcus faecalis |
| 788 | E3M10000038E11 | Enterococcus faecalis |
| 789 | E3M10000038F02 | Enterococcus faecalis |
| 790 | E3M10000038F04 | Enterococcus faecalis |
| 791 | E3M10000038F05 | Enterococcus faecalis |
| 792 | E3M10000038F06 | Enterococcus faecalis |
| 793 | E3M10000038F07 | Enterococcus faecalis |
| 794 | E3M10000038F09 | Enterococcus faecalis |
| 795 | E3M10000038F10 | Enterococcus faecalis |
| 796 | E3M10000038F11 | Enterococcus faecalis |
| 797 | E3M10000038G02 | Enterococcus faecalis |
| 798 | E3M10000038G03 | Enterococcus faecalis |
| 799 | E3M10000038G06 | Enterococcus faecalis |
| 800 | E3M10000038G07 | Enterococcus faecalis |
| 801 | E3M10000038G11 | Enterococcus faecalis |
| 802 | E3M10000038H02 | Enterococcus faecalis |
| 803 | E3M10000038H05 | Enterococcus faecalis |
| 804 | E3M10000038H06 | Enterococcus faecalis |
| 805 | E3M10000038H07 | Enterococcus faecalis |
| 806 | E3M10000038H08 | Enterococcus faecalis |
| 807 | E3M10000038H09 | Enterococcus faecalis |
| 808 | E3M10000038H10 | Enterococcus faecalis |
| 809 | E3M10000039A02 | Enterococcus faecalis |
| 810 | E3M10000039A06 | Enterococcus faecalis |
| 811 | E3M10000039A07 | Enterococcus faecalis |
| 812 | E3M10000039A08 | Enterococcus faecalis |
| 813 | E3M10000039A10 | Enterococcus faecalis |
| 814 | E3M10000039A11 | Enterococcus faecalis |
| 815 | E3M10000039B01 | Enterococcus faecalis |
| 816 | E3M10000039B03 | Enterococcus faecalis |
| 817 | E3M10000039B04 | Enterococcus faecalis |
| 818 | E3M10000039B06 | Enterococcus faecalis |
| 819 | E3M10000039B07 | Enterococcus faecalis |
| 820 | E3M10000039B08 | Enterococcus faecalis |
| 821 | E3M10000039B09 | Enterococcus faecalis |
| 822 | E3M10000039B11 | Enterococcus faecalis |
| 823 | E3M10000039C02 | Enterococcus faecalis |
| 824 | E3M10000039C04 | Enterococcus faecalis |
| 825 | E3M10000039C05 | Enterococcus faecalis |
| 826 | E3M10000039C06 | Enterococcus faecalis |
| 827 | E3M10000039C07 | Enterococcus faecalis |
| 828 | E3M10000039C08 | Enterococcus faecalis |
| 829 | E3M10000039C09 | Enterococcus faecalis |
| 830 | E3M10000039C10 | Enterococcus faecalis |
| 831 | E3M10000039D02 | Enterococcus faecalis |
| 832 | E3M10000039D03 | Enterococcus faecalis |
| 833 | E3M10000039D04 | Enterococcus faecalis |
| 834 | E3M10000039D06 | Enterococcus faecalis |
| 835 | E3M10000039E01 | Enterococcus faecalis |
| 836 | E3M10000039E02 | Enterococcus faecalis |
| 837 | E3M10000039E03 | Enterococcus faecalis |
| 838 | E3M10000039E05 | Enterococcus faecalis |
| 839 | E3M10000039E07 | Enterococcus faecalis |
| 840 | E3M10000039E08 | Enterococcus faecalis |
| 841 | E3M10000039F01 | Enterococcus faecalis |
| 842 | E3M10000039F02 | Enterococcus faecalis |
| 843 | E3M10000039F03 | Enterococcus faecalis |
| 844 | E3M10000039F06 | Enterococcus faecalis |
| 845 | E3M10000039F07 | Enterococcus faecalis |
| 846 | E3M10000039F08 | Enterococcus faecalis |
| 847 | E3M10000039G01 | Enterococcus faecalis |
| 848 | E3M10000039G02 | Enterococcus faecalis |
| 849 | E3M10000039G05 | Enterococcus faecalis |
| 850 | E3M10000039G07 | Enterococcus faecalis |
| 851 | E3M10000039G09 | Enterococcus faecalis |
| 852 | E3M10000039G10 | Enterococcus faecalis |
| 853 | E3M10000039H02 | Enterococcus faecalis |
| 854 | E3M10000039H07 | Enterococcus faecalis |
| 855 | E3M10000039H08 | Enterococcus faecalis |
| 856 | E3M10000039H10 | Enterococcus faecalis |
| 857 | E3M10000039H11 | Enterococcus faecalis |
| 858 | E3M10000040A03 | Enterococcus faecalis |
| 859 | E3M10000040A05 | Enterococcus faecalis |
| 860 | E3M10000040A07 | Enterococcus faecalis |
| 861 | E3M10000040A09 | Enterococcus faecalis |
| 862 | E3M10000040A10 | Enterococcus faecalis |
| 863 | E3M10000040A11 | Enterococcus faecalis |
| 864 | E3M10000040B01 | Enterococcus faecalis |
| 865 | E3M10000040B02 | Enterococcus faecalis |
| 866 | E3M10000040B05 | Enterococcus faecalis |
| 867 | E3M10000040B06 | Enterococcus faecalis |
| 868 | E3M10000040B08 | Enterococcus faecalis |
| 869 | E3M10000040B09 | Enterococcus faecalis |
| 870 | E3M1000004GB10 | Enterococcus faecalis |
| 871 | E3M1000004GB11 | Enterococcus faecalis |
| 872 | E3M1000004GB12 | Enterococcus faecalis |
| 873 | E3M10000040C02 | Enterococcus faecalis |
| 874 | E3M10000040C05 | Enterococcus faecalis |
| 875 | E3M10000040C06 | Enterococcus faecalis |
| 876 | E3M10000040C07 | Enterococcus faecalis |
| 877 | E3M10000040C08 | Enterococcus faecalis |
| 878 | E3M10000040C09 | Enterococcus faecalis |
| 879 | E3M10000040C10 | Enterococcus faecalis |
| 880 | E3M10000040C11 | Enterococcus faecalis |
| 881 | E3M10000040C12 | Enterococcus faecalis |
| 882 | E3M10000040D03 | Enterococcus faecalis |
| 883 | E3M10000040D04 | Enterococcus faecalis |
| 884 | E3M10000040D08 | Enterococcus faecalis |
| 885 | E3M10000040D12 | Enterococcus faecalis |
| 886 | E3M10000040E02 | Enterococcus faecalis |
| 887 | E3M10000040E10 | Enterococcus faecalis |
| 888 | E3M10000040E11 | Enterococcus faecalis |
| 889 | E3M10000040E12 | Enterococcus faecalis |
| 890 | E3M10000040F01 | Enterococcus faecalis |
| 891 | E3M10000040F03 | Enterococcus faecalis |
| 892 | E3M10000040F08 | Enterococcus faecalis |
| 893 | E3M10000040F09 | Enterococcus faecalis |
| 894 | E3M10000040F10 | Enterococcus faecalis |
| 895 | E3M10000040G01 | Enterococcus faecalis |
| 896 | E3M10000040G02 | Enterococcus faecalis |
| 897 | E3M10000040G04 | Enterococcus faecalis |
| 898 | E3M10000040G05 | Enterococcus faecalis |
| 899 | E3M10000040G07 | Enterococcus faecalis |
| 900 | E3M10000040G08 | Enterococcus faecalis |
| 901 | E3M10000040G09 | Enterococcus faecalis |
| 902 | E3M10000040G11 | Enterococcus faecalis |
| 903 | E3M10000040H02 | Enterococcus faecalis |
| 904 | E3M10000040H03 | Enterococcus faecalis |
| 905 | E3M10000040H04 | Enterococcus faecalis |
| 906 | E3M10000040H05 | Enterococcus faecalis |
| 907 | E3M10000040H09 | Enterococcus faecalis |
| 908 | E3M10000041A03 | Enterococcus faecalis |
| 909 | E3M10000041A05 | Enterococcus faecalis |
| 910 | E3M10000041A08 | Enterococcus faecalis |
| 911 | E3M10000041A09 | Enterococcus faecalis |
| 912 | E3M10000041A10 | Enterococcus faecalis |
| 913 | E3M10000041A11 | Enterococcus faecalis |
| 914 | E3M10000041B02 | Enterococcus faecalis |
| 915 | E3M10000041B03 | Enterococcus faecalis |
| 916 | E3M10000041B05 | Enterococcus faecalis |
| 917 | E3M10000041B06 | Enterococcus faecalis |
| 918 | E3M10000041B08 | Enterococcus faecalis |
| 919 | E3M10000041B09 | Enterococcus faecalis |
| 920 | E3M10000041B10 | Enterococcus faecalis |
| 921 | E3M10000041B11 | Enterococcus faecalis |
| 922 | E3M10000041B12 | Enterococcus faecalis |
| 923 | E3M10000041C01 | Enterococcus faecalis |
| 924 | E3M10000041C07 | Enterococcus faecalis |
| 925 | E3M10000041C08 | Enterococcus faecalis |
| 926 | E3M10000041C09 | Enterococcus faecalis |
| 927 | E3M10000041C10 | Enterococcus faecalis |
| 928 | E3M10000041C11 | Enterococcus faecalis |
| 929 | E3M10000041C12 | Enterococcus faecalis |
| 930 | E3M10000041D02 | Enterococcus faecalis |
| 931 | E3M10000041D03 | Enterococcus faecalis |
| 932 | E3M10000041D04 | Enterococcus faecalis |
| 933 | E3M10000041D05 | Enterococcus faecalis |
| 934 | E3M10000041D06 | Enterococcus faecalis |
| 935 | E3M10000041D08 | Enterococcus faecalis |
| 936 | E3M10000041D09 | Enterococcus faecalis |
| 937 | E3M10000041D10 | Enterococcus faecalis |
| 938 | E3M10000041D11 | Enterococcus faecalis |
| 939 | E3M10000041D12 | Enterococcus faecalis |
| 940 | E3M10000041E02 | Enterococcus faecalis |
| 941 | E3M10000041E03 | Enterococcus faecalis |
| 942 | E3M10000041EO5 | Enterococcus faecalis |
| 943 | E3M10000041E07 | Enterococcus faecalis |
| 944 | E3M10000041E10 | Enterococcus faecalis |
| 945 | E3M1000004IE11 | Enterococcus faecalis |
| 946 | E3M10000041F03 | Enterococcus faecalis |
| 947 | E3M10000041F05 | Enterococcus faecalis |
| 948 | E3M10000041F06 | Enterococcus faecalis |
| 949 | E3M10000041F07 | Enterococcus faecalis |
| 950 | E3M10000041F08 | Enterococcus faecalis |
| 951 | E3M10000041F09 | Enterococcus faecalis |
| 952 | E3M10000041F10 | Enterococcus faecalis |
| 953 | E3M10000041F11 | Enterococcus faecalis |
| 954 | E3M10000041G02 | Enterococcus faecalis |
| 955 | E3M10000041G03 | Enterococcus faecalis |
| 956 | E3M10000041G04 | Enterococcus faecalis |
| 957 | E3M10000041G06 | Enterococcus faecalis |
| 958 | E3M10000041G07 | Enterococcus faecalis |
| 959 | E3M10000041G08 | Enterococcus faecalis |
| 960 | E3M10000041G09 | Enterococcus faecalis |
| 961 | E3M10000041G10 | Enterococcus faecalis |
| 962 | E3M10000041G12 | Enterococcus faecalis |
| 963 | E3M10000041H04 | Enterococcus faecalis |
| 964 | E3M10000041H05 | Enterococcus faecalis |
| 965 | E3M10000041H06 | Enterococcus faecalis |
| 966 | E3M10000041H07 | Enterococcus faecalis |
| 967 | E3M10000041H08 | Enterococcus faecalis |
| 968 | E3M10000041H09 | Enterococcus faecalis |
| 969 | E3M10000041H10 | Enterococcus faecalis |
| 970 | E3M10000041H11 | Enterococcus faecalis |
| 971 | E3M10000042A03 | Enterococcus faecalis |
| 972 | E3M10000042A08 | Enterococcus faecalis |
| 973 | E3M10000042A10 | Enterococcus faecalis |
| 974 | E3M10000042B01 | Enterococcus faecalis |
| 975 | E3M10000042B02 | Enterococcus faecalis |
| 976 | E3M10000042B04 | Enterococcus faecalis |
| 977 | E3M10000042B08 | Enterococcus faecalis |
| 978 | E3M10000042B09 | Enterococcus faecalis |
| 979 | E3M10000042B10 | Enterococcus faecalis |
| 980 | E3M10000042B11 | Enterococcus faecalis |
| 981 | E3M10000042C02 | Enterococcus faecalis |
| 982 | E3M10000042C03 | Enterococcus faecalis |
| 983 | E3M10000042C04 | Enterococcus faecalis |
| 984 | E3M10000042C10 | Enterococcus faecalis |
| 985 | E3M10000042D01 | Enterococcus faecalis |
| 986 | E3M10000042D02 | Enterococcus faecalis |
| 987 | E3M10000042D03 | Enterococcus faecalis |
| 988 | E3M10000042D06 | Enterococcus faecalis |
| 989 | E3M10000042D09 | Enterococcus faecalis |
| 990 | E3M10000042D11 | Enterococcus faecalis |
| 991 | E3M10000042D12 | Enterococcus faecalis |
| 992 | E3M10000042E05 | Enterococcus faecalis |
| 993 | E3M10000042E12 | Enterococcus faecalis |
| 994 | E3M10000042F11 | Enterococcus faecalis |
| 995 | E3M10000042G01 | Enterococcus faecalis |
| 996 | E3M10000042G05 | Enterococcus faecalis |
| 997 | E3M10000042G07 | Enterococcus faecalis |
| 998 | E3M10000042G08 | Enterococcus faecalis |
| 999 | E3M10000042G11 | Enterococcus faecalis |
| 1000 | E3M10000042G12 | Enterococcus faecalis |
| 1001 | E3M10000042H06 | Enterococcus faecalis |
| 1002 | E3M10000042H08 | Enterococcus faecalis |
| 1003 | E3M10000042H11 | Enterococcus faecalis |
| 1004 | E3M10000043A02 | Enterococcus faecalis |
| 1005 | E3M10000043A03 | Enterococcus faecalis |
| 1006 | E3M10000043A05 | Enterococcus faecalis |
| 1007 | E3M10000043A08 | Enterococcus faecalis |
| 1008 | E3M10000043A09 | Enterococcus faecalis |
| 1009 | E3M10000043A10 | Enterococcus faecalis |
| 1010 | E3M10000043A11 | Enterococcus faecalis |
| 1011 | E3M10000043B01 | Enterococcus faecalis |
| 1012 | E3M10000043B02 | Enterococcus faecalis |
| 1013 | E3M10000043B03 | Enterococcus faecalis |
| 1014 | E3M10000043B06 | Enterococcus faecalis |
| 1015 | E3M10000043B08 | Enterococcus faecalis |
| 1016 | E3M10000043B09 | Enterococcus faecalis |
| 1017 | E3M10000043B10 | Enterococcus faecalis |
| 1018 | E3M10000043B11 | Enterococcus faecalis |
| 1019 | E3M10000043B12 | Enterococcus faecalis |
| 1020 | E3M10000043C01 | Enterococcus faecalis |
| 1021 | E3M10000043C08 | Enterococcus faecalis |
| 1022 | E3M10000043C09 | Enterococcus faecalis |
| 1023 | E3M10000043D01 | Enterococcus faecalis |
| 1024 | E3M10000043D02 | Enterococcus faecalis |
| 1025 | E3M10000043D09 | Enterococcus faecalis |
| 1026 | E3M10000043D10 | Enterococcus faecalis |
| 1027 | E3M10000043D12 | Enterococcus faecalis |
| 1028 | E3M10000043E03 | Enterococcus faecalis |
| 1029 | E3M10000043E07 | Enterococcus faecalis |
| 1030 | E3M10000043E08 | Enterococcus faecalis |
| 1031 | E3M10000043E10 | Enterococcus faecalis |
| 1032 | E3M10000043E11 | Enterococcus faecalis |
| 1033 | E3M10000043F03 | Enterococcus faecalis |
| 1034 | E3M10000043F04 | Enterococcus faecalis |
| 1035 | E3M10000043F06 | Enterococcus faecalis |
| 1036 | E3M10000043F08 | Enterococcus faecalis |
| 1037 | E3M10000043F10 | Enterococcus faecalis |
| 1038 | E3M10000043F12 | Enterococcus faecalis |
| 1039 | E3M10000043G03 | Enterococcus faecalis |
| 1040 | E3M10000043G04 | Enterococcus faecalis |
| 1041 | E3M10000043G05 | Enterococcus faecalis |
| 1042 | E3M10000043G07 | Enterococcus faecalis |
| 1043 | E3M10000043G08 | Enterococcus faecalis |
| 1044 | E3M10000043G10 | Enterococcus faecalis |
| 1045 | E3M10000043G11 | Enterococcus faecalis |
| 1046 | E3M10000043G12 | Enterococcus faecalis |
| 1047 | E3M10000043H02 | Enterococcus faecalis |
| 1048 | E3M10000043H05 | Enterococcus faecalis |
| 1049 | E3M10000043H08 | Enterococcus faecalis |
| 1050 | E3M10000043H09 | Enterococcus faecalis |
| 1051 | E3M10000043H11 | Enterococcus faecalis |
| 1052 | E3M10000044C02 | Enterococcus faecalis |
| 1053 | E3M10000044E01 | Enterococcus faecalis |
| 1054 | K1M10000002F02 | Klebsiella pneumoniae |
| 1055 | K1M10000003C01 | Klebsiella pneumoniae |
| 1056 | K1M10000004F06 | Klebsiella pneumoniae |
| 1057 | K1M10000007F01 | Klebsiella pneumoniae |
| 1058 | K1M10000008C02 | Klebsiella pneumoniae |
| 1059 | K1M10000008C10 | Klebsiella pneumoniae |
| 1060 | K1M10000008G10 | Klebsiella pneumoniae |
| 1061 | K1M10000009D04 | Klebsiella pneumoniae |
| 1062 | K1M10000013E04 | Klebsiella pneumoniae |
| 1063 | K1M10000013E06 | Klebsiella pneumoniae |
| 1064 | K1M10000019D06 | Klebsiella pneumoniae |
| 1065 | K1M10000020B02 | Klebsiella pneumoniae |
| 1066 | K1M10000021HO6 | Klebsiella pneumoniae |
| 1067 | K1M10000022C10 | Klebsiella pneumoniae |
| 1068 | K1M10000023E09 | Klebsiella pneumoniae |
| 1069 | K1M10000023E10 | Klebsiella pneumoniae |
| 1070 | K1M10000030C07 | Klebsiella pneumoniae |
| 1071 | K1M10000030E07 | Klebsiella pneumoniae |
| 1072 | K1M10000031B11 | Klebsiella pneumoniae |
| 1073 | K1M10000032E11 | Klebsiella pneumoniae |
| 1074 | K1M10000033B02 | Klebsiella pneumoniae |
| 1075 | K1M10000033E01 | Klebsiella pneumoniae |
| 1076 | K1M10000036G08 | Klebsiella pneumoniae |
| 1077 | K1M10000037D10 | Klebsiella pneumoniae |
| 1078 | K1M10000038H09 | Klebsiella pneumoniae |
| 1079 | K1M10000039H03 | Klebsiella pneumoniae |
| 1080 | K1M10000043C01 | Klebsiella pneumoniae |
| 1081 | K1M10000043D05 | Klebsiella pneumoniae |
| 1082 | K1M10000043H10 | Klebsiella pneumoniae |
| 1083 | K1M10000044D05 | Klebsiella pneumoniae |
| 1084 | K1M10000044D08 | Klebsiella pneumoniae |
| 1085 | K1M10000044E05 | Klebsiella pneumoniae |
| 1086 | K1M10000044G05 | Klebsiella pneumoniae |
| 1087 | K1M10000045A07 | Klebsiella pneumoniae |
| 1088 | K1M10000045D10 | Klebsiella pneumoniae |
| 1089 | K1M10000003D03 | Klebsiella pneumoniae |
| 1090 | K1M10000010C02 | Klebsiella pneumoniae |
| 1091 | K1M10000021H10 | Klebsiella pneumoniae |
| 1092 | P1M10000008C06 | Pseudomonas aeruginosa |
| 1093 | P1M10000008G04 | Pseudomonas aeruginosa |
| 1094 | P1M10000010C03 | Pseudomonas aeruginosa |
| 1095 | P1M10000014H10 | Pseudomonas aeruginosa |
| 1096 | P1M10000015C06 | Pseudomonas aeruginosa |
| 1097 | P1M10000015C09 | Pseudomonas aeruginosa |
| 1098 | P1M10000016C04 | Pseudomonas aeruginosa |
| 1099 | P1M10000018B01 | Pseudomonas aeruginosa |
| 1100 | P1M10000018C01 | Pseudomonas aeruginosa |
| 1101 | P1M10000018E01 | Pseudomonas aeruginosa |
| 1102 | P1M10000018G01 | Pseudomonas aeruginosa |
| 1103 | P1M10000019F01 | Pseudomonas aeruginosa |
| 1104 | P1M10000021G03 | Pseudomonas aeruginosa |
| 1105 | P1M10000021G05 | Pseudomonas aeruginosa |
| 1106 | P1M10000022D09 | Pseudomonas aeruginosa |
| 1107 | P1M10000024D06 | Pseudomonas aeruginosa |
| 1108 | P1M10000024E06 | Pseudomonas aeruginosa |
| 1109 | P1M10000024H03 | Pseudomonas aeruginosa |
| 1110 | P1M10000025A06 | Pseudomonas aeruginosa |
| 1111 | P1M10000025G07 | Pseudomonas aeruginosa |
| 1112 | P1M10000025H07 | Pseudomonas aeruginosa |
| 1113 | P1M10000026E06 | Pseudomonas aeruginosa |
| 1114 | P1M10000026F04 | Pseudomonas aeruginosa |
| 1115 | P1M10000026G09 | Pseudomonas aeruginosa |
| 1116 | P1M10000026H02 | Pseudomonas aeruginosa |
| 1117 | P1M10000026H05 | Pseudomonas aeruginosa |
| 1118 | P1M10000027A06 | Pseudomonas aeruginosa |
| 1119 | P1M10000027B02 | Pseudomonas aeruginosa |
| 1120 | P1M10000027G05 | Pseudomonas aeruginosa |
| 1121 | P1M10000028A08 | Pseudomonas aeruginosa |
| 1122 | P1M10000028B01 | Pseudomonas aeruginosa |
| 1123 | P1M10000028E02 | Pseudomonas aeruginosa |
| 1124 | P1M10000029A09 | Pseudomonas aeruginosa |
| 1125 | P1M10000029G03 | Pseudomonas aeruginosa |
| 1126 | P1M10000029H05 | Pseudomonas aeruginosa |
| 1127 | P1M10000032F04 | Pseudomonas aeruginosa |
| 1128 | P1M10000033A02 | Pseudomonas aeruginosa |
| 1129 | P1M10000033B08 | Pseudomonas aeruginosa |
| 1130 | P1M10000033E03 | Pseudomonas aeruginosa |
| 1131 | P1M10000033F01 | Pseudomonas aeruginosa |
| 1132 | P1M10000033G08 | Pseudomonas aeruginosa |
| 1133 | P1M10000035A06 | Pseudomonas aeruginosa |
| 1134 | P1M10000037B12 | Pseudomonas aeruginosa |
| 1135 | P1M10000037G12 | Pseudomonas aeruginosa |
| 1136 | P1M10000038B08 | Pseudomonas aeruginosa |
| 1137 | P1M10000038C03 | Pseudomonas aeruginosa |
| 1138 | P1M10000038C06 | Pseudomonas aeruginosa |
| 1139 | P1M10000038F04 | Pseudomonas aeruginosa |
| 1140 | P1M10000038G02 | Pseudomonas aeruginosa |
| 1141 | P1M10000039G05 | Pseudomonas aeruginosa |
| 1142 | P1M10000039G12 | Pseudomonas aeruginosa |
| 1143 | PIM10000040C01 | Pseudomonas aeruginosa |
| 1144 | P1M10000040C04 | Pseudomonas aeruginosa |
| 1145 | P1M10000040D04 | Pseudomonas aeruginosa |
| 1146 | P1M10000040D05 | Pseudomonas aeruginosa |
| 1147 | P1M10000040E10 | Pseudomonas aeruginosa |
| 1148 | P1M10000040H03 | Pseudomonas aeruginosa |
| 1149 | P1M1OOOOO41A12 | Pseudomonas aeruginosa |
| 1150 | P1M10000041B02 | Pseudomonas aeruginosa |
| 1151 | P1M10000041E01 | Pseudomonas aeruginosa |
| 1152 | P1M10000041F01 | Pseudomonas aeruginosa |
| 1153 | P1M10000042B12 | Pseudomonas aeruginosa |
| 1154 | P1M10000042E08 | Pseudomonas aeruginosa |
| 1155 | P1M10000043A03 | Pseudomonas aeruginosa |
| 1156 | P1M10000043D06 | Pseudomonas aeruginosa |
| 1157 | P1M10000044F07 | Pseudomonas aeruginosa |
| 1158 | P1M10000046B03 | Pseudomonas aeruginosa |
| 1159 | P1M10000046C07 | Pseudomonas aeruginosa |
| 1160 | P1M10000046C08 | Pseudomonas aeruginosa |
| 1161 | P1M10000046C09 | Pseudomonas aeruginosa |
| 1162 | P1M10000046G11 | Pseudomonas aeruginosa |
| 1163 | P1M10000047B04 | Pseudomonas aeruginosa |
| 1164 | P1M10000047E11 | Pseudomonas aeruginosa |
| 1165 | P1M10000047F07 | Pseudomonas aeruginosa |
| 1166 | P1M10000047G10 | Pseudomonas aeruginosa |
| 1167 | P1M10000048A03 | Pseudomonas aeruginosa |
| 1168 | P1M10000049E08 | Pseudomonas aeruginosa |
| 1169 | P1M10000049G10 | Pseudomonas aeruginosa |
| 1170 | P1M10000050G11 | Pseudomonas aeruginosa |
| 1171 | P1M10000051D11 | Pseudomonas aeruginosa |
| 1172 | P1M10000051F01 | Pseudomonas aeruginosa |
| 1173 | P1M10000052C03 | Pseudomonas aeruginosa |
| 1174 | P1M10000052C12 | Pseudomonas aeruginosa |
| 1175 | P1M10000052E04 | Pseudomonas aeruginosa |
| 1176 | P1M10000053B12 | Pseudomonas aeruginosa |
| 1177 | P1M10000053C02 | Pseudomonas aeruginosa |
| 1178 | P1M10000053E07 | Pseudomonas aeruginosa |
| 1179 | P1M10000053F08 | Pseudomonas aeruginosa |
| 1180 | P1M10000055A11 | Pseudomonas aeruginosa |
| 1181 | P1M10000055C08 | Pseudomonas aeruginosa |
| 1182 | P1M10000055E05 | Pseudomonas aeruginosa |
| 1183 | P1M10000056C07 | Pseudomonas aeruginosa |
| 1184 | P1M10000056F05 | Pseudomonas aeruginosa |
| 1185 | P1M10000056F06 | Pseudomonas aeruginosa |
| 1186 | P1M10000056G01 | Pseudomonas aeruginosa |
| 1187 | P1M10000058B07 | Pseudomonas aeruginosa |
| 1188 | P1M10000059B04 | Pseudomonas aeruginosa |
| 1189 | P1M10000059B10 | Pseudomonas aeruginosa |
| 1190 | P1M10000059B11 | Pseudomonas aeruginosa |
| 1191 | P1M10000059D11 | Pseudomonas aeruginosa |
| 1192 | P1M10000059H08 | Pseudomonas aeruginosa |
| 1193 | P1M10000059H09 | Pseudomonas aeruginosa |
| 1194 | P1M10000060E03 | Pseudomonas aeruginosa |
| 1195 | P1M10000060H02 | Pseudomonas aeruginosa |
| 1196 | P1M10000060H04 | Pseudomonas aeruginosa |
| 1197 | P1M10000061B04 | Pseudomonas aeruginosa |
| 1198 | P1M10000061E04 | Pseudomonas aeruginosa |
| 1199 | P1M10000061F04 | Pseudomonas aeruginosa |
| 1200 | P1M10000062A12 | Pseudomonas aeruginosa |
| 1201 | P1M10000062C03 | Pseudomonas aeruginosa |
| 1202 | P1M10000062C04 | Pseudomonas aeruginosa |
| 1203 | P1M10000062C07 | Pseudomonas aeruginosa |
| 1204 | P1M10000062C12 | Pseudomonas aeruginosa |
| 1205 | P1M10000062D07 | Pseudomonas aeruginosa |
| 1206 | P1M10000062D08 | Pseudomonas aeruginosa |
| 1207 | P1M10000062E08 | Pseudomonas aeruginosa |
| 1208 | P1M10000062F06 | Pseudomonas aeruginosa |
| 1209 | P1M10000062G11 | Pseudomonas aeruginosa |
| 1210 | P1M10000062H01 | Pseudomonas aeruginosa |
| 1211 | P1M10000062H04 | Pseudomonas aeruginosa |
| 1212 | P1M10000063F02 | Pseudomonas aeruginosa |
| 1213 | P1M10000063G02 | Pseudomonas aeruginosa |
| 1214 | P1M10000063H02 | Pseudomonas aeruginosa |
| 1215 | P1M10000064A10 | Pseudomonas aeruginosa |
| 1216 | P1M10000064C02 | Pseudomonas aeruginosa |
| 1217 | P1M10000064C03 | Pseudomonas aeruginosa |
| 1218 | P1M10000064D03 | Pseudomonas aeruginosa |
| 1219 | P1M10000064E05 | Pseudomonas aeruginosa |
| 1220 | P1M10000064G12 | Pseudomonas aeruginosa |
| 1221 | P1M10000064H07 | Pseudomonas aeruginosa |
| 1222 | P1M10000065A04 | Pseudomonas aeruginosa |
| 1223 | P1M10000065B07 | Pseudomonas aeruginosa |
| 1224 | P1M10000065C03 | Pseudomonas aeruginosa |
| 1225 | P1M10000065C05 | Pseudomonas aeruginosa |
| 1226 | P1M10000065D06 | Pseudomonas aeruginosa |
| 1227 | P1M10000065F01 | Pseudomonas aeruginosa |
| 1228 | P1M10000065G06 | Pseudomonas aeruginosa |
| 1229 | P1M10000065H07 | Pseudomonas aeruginosa |
| 1230 | P1M10000066A10 | Pseudomonas aeruginosa |
| 1231 | P1M10000066A11 | Pseudomonas aeruginosa |
| 1232 | P1M10000066F04 | Pseudomonas aeruginosa |
| 1233 | P1M10000067A05 | Pseudomonas aeruginosa |
| 1234 | P1M10000067A06 | Pseudomonas aeruginosa |
| 1235 | P1M10000067A08 | Pseudomonas aeruginosa |
| 1236 | P1M10000067C04 | Pseudomonas aeruginosa |
| 1237 | P1M10000067C06 | Pseudomonas aeruginosa |
| 1238 | P1M10000067D05 | Pseudomonas aeruginosa |
| 1239 | P1M10000067F05 | Pseudomonas aeruginosa |
| 1240 | P1M10000067G05 | Pseudomonas aeruginosa |
| 1241 | P1M10000068A09 | Pseudomonas aeruginosa |
| 1242 | P1M10000068D04 | Pseudomonas aeruginosa |
| 1243 | P1M10000068F04 | Pseudomonas aeruginosa |
| 1244 | P1M10000068F08 | Pseudomonas aeruginosa |
| 1245 | P1M10000068G01 | Pseudomonas aeruginosa |
| 1246 | P1M10000068H05 | Pseudomonas aeruginosa |
| 1247 | P1M10000069D09 | Pseudomonas aeruginosa |
| 1248 | P1M10000069G06 | Pseudomonas aeruginosa |
| 1249 | P1M10000069H02 | Pseudomonas aeruginosa |
| 1250 | P1M10000070A05 | Pseudomonas aeruginosa |
| 1251 | P1M10000070B10 | Pseudomonas aeruginosa |
| 1252 | P1M10000070C06 | Pseudomonas aeruginosa |
| 1253 | P1M10000070D08 | Pseudomonas aeruginosa |
| 1254 | P1M10000070E03 | Pseudomonas aeruginosa |
| 1255 | P1M10000070G06 | Pseudomonas aeruginosa |
| 1256 | P1M10000070G12 | Pseudomonas aeruginosa |
| 1257 | P1M10000070H06 | Pseudomonas aeruginosa |
| 1258 | P1M10000071A03 | Pseudomonas aeruginosa |
| 1259 | P1M10000071C01 | Pseudomonas aeruginosa |
| 1260 | P1M10000071E04 | Pseudomonas aeruginosa |
| 1261 | P1M10000071F01 | Pseudomonas aeruginosa |
| 1262 | P1M10000073A06 | Pseudomonas aeruginosa |
| 1263 | P1M10000073B10 | Pseudomonas aeruginosa |
| 1264 | P1M10000073D04 | Pseudomonas aeruginosa |
| 1265 | P1M10000073D09 | Pseudomonas aeruginosa |
| 1266 | P1M10000073G03 | Pseudomonas aeruginosa |
| 1267 | PIM10000074B01 | Pseudomonas aeruginosa |
| 1268 | P1M10000074B04 | Pseudomonas aeruginosa |
| 1269 | P1M10000074E04 | Pseudomonas aeruginosa |
| 1270 | P1M10000074E09 | Pseudomonas aeruginosa |
| 1271 | P1M10000074F10 | Pseudomonas aeruginosa |
| 1272 | P1M10000074G12 | Pseudomonas aeruginosa |
| 1273 | P1M10000075A04 | Pseudomonas aeruginosa |
| 1274 | P1M10000075B03 | Pseudomonas aeruginosa |
| 1275 | P1M10000075F02 | Pseudomonas aeruginosa |
| 1276 | P1M10000075G05 | Pseudomonas aeruginosa |
| 1277 | P1M10000076D05 | Pseudomonas aeruginosa |
| 1278 | P1M10000076D10 | Pseudomonas aeruginosa |
| 1279 | P1M10000077A08 | Pseudomonas aeruginosa |
| 1280 | P1M10000077C08 | Pseudomonas aeruginosa |
| 1281 | P1M10000077E04 | Pseudomonas aeruginosa |
| 1282 | P1M10000077H05 | Pseudomonas aeruginosa |
| 1283 | P1M10000079A10 | Pseudomonas aeruginosa |
| 1284 | P1M10000079B10 | Pseudomonas aeruginosa |
| 1285 | P1M10000079C10 | Pseudomonas aeruginosa |
| 1286 | P1M10000079D01 | Pseudomonas aeruginosa |
| 1287 | P1M10000079D10 | Pseudomonas aeruginosa |
| 1288 | P1M10000079F06 | Pseudomonas aeruginosa |
| 1289 | P1M10000080B01 | Pseudomonas aeruginosa |
| 1290 | P1M10000080B06 | Pseudomonas aeruginosa |
| 1291 | P1M10000080C01 | Pseudomonas aeruginosa |
| 1292 | P1M10000080C06 | Pseudomonas aeruginosa |
| 1293 | P1M10000080E04 | Pseudomonas aeruginosa |
| 1294 | P1M10000081D12 | Pseudomonas aeruginosa |
| 1295 | P1M10000081G05 | Pseudomonas aeruginosa |
| 1296 | P1M10000081H05 | Pseudomonas aeruginosa |
| 1297 | P1M10000082A05 | Pseudomonas aeruginosa |
| 1298 | P1M10000082B04 | Pseudomonas aeruginosa |
| 1299 | P1M10000082C05 | Pseudomonas aeruginosa |
| 1300 | P1M10000082D05 | Pseudomonas aeruginosa |
| 1301 | P1M10000082E05 | Pseudomonas aeruginosa |
| 1302 | P1M10000083A11 | Pseudomonas aeruginosa |
| 1303 | P1M10000083B01 | Pseudomonas aeruginosa |
| 1304 | P1M10000083B12 | Pseudomonas aeruginosa |
| 1305 | P1M10000083C11 | Pseudomonas aeruginosa |
| 1306 | P1M10000083C12 | Pseudomonas aeruginosa |
| 1307 | P1M10000084A04 | Pseudomonas aeruginosa |
| 1308 | P1M10000084D03 | Pseudomonas aeruginosa |
| 1309 | P1M10000084E04 | Pseudomonas aeruginosa |
| 1310 | P1M10000084E11 | Pseudomonas aeruginosa |
| 1311 | P1M10000084F08 | Pseudomonas aeruginosa |
| 1312 | P1M10000085D06 | Pseudomonas aeruginosa |
| 1313 | P1M10000086A02 | Pseudomonas aeruginosa |
| 1314 | P1M10000086B01 | Pseudomonas aeruginosa |
| 1315 | P1M10000086D02 | Pseudomonas aeruginosa |
| 1316 | P1M10000086E05 | Pseudomonas aeruginosa |
| 1317 | P1M10000087A11 | Pseudomonas aeruginosa |
| 1318 | P1M10000087C09 | Pseudomonas aeruginosa |
| 1319 | P1M10000087E04 | Pseudomonas aeruginosa |
| 1320 | P1M10000087F04 | Pseudomonas aeruginosa |
| 1321 | P1M10000087F09 | Pseudomonas aeruginosa |
| 1322 | P1M10000088A07 | Pseudomonas aeruginosa |
| 1323 | P1M10000088D06 | Pseudomonas aeruginosa |
| 1324 | P1M10000089C08 | Pseudomonas aeruginosa |
| 1325 | P1M10000089D11 | Pseudomonas aeruginosa |
| 1326 | P1M10000089G08 | Pseudomonas aeruginosa |
| 1327 | P1M10000090B11 | Pseudomonas aeruginosa |
| 1328 | P1M10000090F06 | Pseudomonas aeruginosa |
| 1329 | P1M10000090F08 | Pseudomonas aeruginosa |
| 1330 | P1M10000091D02 | Pseudomonas aeruginosa |
| 1331 | P1M10000091E09 | Pseudomonas aeruginosa |
| 1332 | P1M10000091G10 | Pseudomonas aeruginosa |
| 1333 | P1M10000092B02 | Pseudomonas aeruginosa |
| 1334 | P1M10000092B10 | Pseudomonas aeruginosa |
| 1335 | P1M10000092D09 | Pseudomonas aeruginosa |
| 1336 | P1M10000092E02 | Pseudomonas aeruginosa |
| 1337 | P1M10000092F05 | Pseudomonas aeruginosa |
| 1338 | P1M10000093A03 | Pseudomonas aeruginosa |
| 1339 | P1M10000093B09 | Pseudomonas aeruginosa |
| 1340 | P1M10000093C08 | Pseudomonas aeruginosa |
| 1341 | P1M10000093E09 | Pseudomonas aeruginosa |
| 1342 | P1M10000093F03 | Pseudomonas aeruginosa |
| 1343 | P1M10000093H07 | Pseudomonas aeruginosa |
| 1344 | P1M10000094F04 | Pseudomonas aeruginosa |
| 1345 | P1M10000094H03 | Pseudomonas aeruginosa |
| 1346 | P1M10000095C01 | Pseudomonas aeruginosa |
| 1347 | P1M10000095C09 | Pseudomonas aeruginosa |
| 1348 | P1M10000095E04 | Pseudomonas aeruginosa |
| 1349 | P1M10000095G04 | Pseudomonas aeruginosa |
| 1350 | P1M10000096E04 | Pseudomonas aeruginosa |
| 1351 | P1M10000096E12 | Pseudomonas aeruginosa |
| 1352 | ID2 | Pseudomonas aeruginosa |
| 1353 | 4.1 | Pseudomonas aeruginosa |
| 1354 | S1M10000001A05 | Staphylococcus aureus |
| 1355 | S1M10000001A08 | Staphylococcus aureus |
| 1356 | S1M10000001A09 | Staphylococcus aureus |
| 1357 | S1M10000001A10 | Staphylococcus aureus |
| 1358 | S1M10000001C06 | Staphylococcus aureus |
| 1359 | S1M10000001D01 | Staphylococcus aureus |
| 1360 | S1M1000000ID02 | Staphylococcus aureus |
| 1361 | S1M1000000ID06 | Staphylococcus aureus |
| 1362 | S1M10000001D07 | Staphylococcus aureus |
| 1363 | S1M10000001E02 | Staphylococcus aureus |
| 1364 | S1M10000001E04 | Staphylococcus aureus |
| 1365 | S1M10000001E05 | Staphylococcus aureus |
| 1366 | S1M10000001E09 | Staphylococcus aureus |
| 1367 | S1M10000001E10 | Staphylococcus aureus |
| 1368 | S1M10000001E11 | Staphylococcus aureus |
| 1369 | S1M10000001F02 | Staphylococcus aureus |
| 1370 | S1M10000001F04 | Staphylococcus aureus |
| 1371 | S1M10000001F08 | Staphylococcus aureus |
| 1372 | S1M10000001F09 | Staphylococcus aureus |
| 1373 | S1M10000001F10 | Staphylococcus aureus |
| 1374 | S1M10000001F11 | Staphylococcus aureus |
| 1375 | S1M10000001G01 | Staphylococcus aureus |
| 1376 | S1M10000001G07 | Staphylococcus aureus |
| 1377 | S1M10000001G08 | Staphylococcus aureus |
| 1378 | S1M10000001G10 | Staphylococcus aureus |
| 1379 | S1M10000002A02 | Staphylococcus aureus |
| 1380 | S1M10000002A09 | Staphylococcus aureus |
| 1381 | S1M10000002A10 | Staphylococcus aureus |
| 1382 | S1M10000002A12 | Staphylococcus aureus |
| 1383 | S1M10000002B01 | Staphylococcus aureus |
| 1384 | S1M10000002B03 | Staphylococcus aureus |
| 1385 | S1M10000002B04 | Staphylococcus aureus |
| 1386 | S1M10000002B05 | Staphylococcus aureus |
| 1387 | S1M10000002B06 | Staphylococcus aureus |
| 1388 | S1M10000002B07 | Staphylococcus aureus |
| 1389 | S1M10000002B09 | Staphylococcus aureus |
| 1390 | S1M10000002B11 | Staphylococcus aureus |
| 1391 | S1M10000002C02 | Staphylococcus aureus |
| 1392 | S1M10000002C09 | Staphylococcus aureus |
| 1393 | S1M10000002C10 | Staphylococcus aureus |
| 1394 | S1M10000002C11 | Staphylococcus aureus |
| 1395 | S1M10000002C12 | Staphylococcus aureus |
| 1396 | S1M10000002D01 | Staphylococcus aureus |
| 1397 | S1M10000002D02 | Staphylococcus aureus |
| 1398 | S1M10000002D03 | Staphylococcus aureus |
| 1399 | S1M10000002D05 | Staphylococcus aureus |
| 1400 | S1M10000002D07 | Staphylococcus aureus |
| 1401 | S1M10000002D08 | Staphylococcus aureus |
| 1402 | S1M10000002D10 | Staphylococcus aureus |
| 1403 | S1M10000002D12 | Staphylococcus aureus |
| 1404 | S1M10000002E01 | Staphylococcus aureus |
| 1405 | S1M10000002E02 | Staphylococcus aureus |
| 1406 | S1M10000002E07 | Staphylococcus aureus |
| 1407 | S1M10000002E09 | Staphylococcus aureus |
| 1408 | S1M10000002E11 | Staphylococcus aureus |
| 1409 | S1M10000002E12 | Staphylococcus aureus |
| 1410 | S1M10000002F01 | Staphylococcus aureus |
| 1411 | S1M10000002F02 | Staphylococcus aureus |
| 1412 | S1M10000002F04 | Staphylococcus aureus |
| 1413 | S1M10000002F09 | Staphylococcus aureus |
| 1414 | S1M10000002F12 | Staphylococcus aureus |
| 1415 | S1M10000002G01 | Staphylococcus aureus |
| 1416 | S1M10000002G03 | Staphylococcus aureus |
| 1417 | S1M10000002G05 | Staphylococcus aureus |
| 1418 | S1M10000002G06 | Staphylococcus aureus |
| 1419 | S1M10000002G07 | Staphylococcus aureus |
| 1420 | S1M10000002G08 | Staphylococcus aureus |
| 1421 | S1M10000002G09 | Staphylococcus aureus |
| 1422 | S1M10000002G10 | Staphylococcus aureus |
| 1423 | S1M10000002G11 | Staphylococcus aureus |
| 1424 | S1M10000002G12 | Staphylococcus aureus |
| 1425 | S1M10000003A01 | Staphylococcus aureus |
| 1426 | S1M10000003A02 | Staphylococcus aureus |
| 1427 | S1M10000003A03 | Staphylococcus aureus |
| 1428 | S1M10000003A04 | Staphylococcus aureus |
| 1429 | S1M10000003A06 | Staphylococcus aureus |
| 1430 | S1M10000003A07 | Staphylococcus aureus |
| 1431 | S1M10000003A08 | Staphylococcus aureus |
| 1432 | S1M10000003A10 | Staphylococcus aureus |
| 1433 | S1M10000003A11 | Staphylococcus aureus |
| 1434 | S1M10000003B06 | Staphylococcus aureus |
| 1435 | S1M10000003B08 | Staphylococcus aureus |
| 1436 | S1M10000003B09 | Staphylococcus aureus |
| 1437 | S1M10000003B12 | Staphylococcus aureus |
| 1438 | S1M10000003C06 | Staphylococcus aureus |
| 1439 | S1M10000003C07 | Staphylococcus aureus |
| 1440 | S1M10000003C10 | Staphylococcus aureus |
| 1441 | S1M10000003C12 | Staphylococcus aureus |
| 1442 | S1M10000003D05 | Staphylococcus aureus |
| 1443 | S1M10000003D06 | Staphylococcus aureus |
| 1444 | S1M10000003D08 | Staphylococcus aureus |
| 1445 | S1M10000003D10 | Staphylococcus aureus |
| 1446 | S1M10000003E07 | Staphylococcus aureus |
| 1447 | S1M10000003E09 | Staphylococcus aureus |
| 1448 | S1M10000003E10 | Staphylococcus aureus |
| 1449 | S1M10000003E11 | Staphylococcus aureus |
| 1450 | S1M10000003F02 | Staphylococcus aureus |
| 1451 | S1M10000003F05 | Staphylococcus aureus |
| 1452 | S1M10000003F06 | Staphylococcus aureus |
| 1453 | S1M10000003F07 | Staphylococcus aureus |
| 1454 | S1M10000003F08 | Staphylococcus aureus |
| 1455 | S1M10000003F12 | Staphylococcus aureus |
| 1456 | S1M10000003G03 | Staphylococcus aureus |
| 1457 | S1M10000003G04 | Staphylococcus aureus |
| 1458 | S1M10000003G08 | Staphylococcus aureus |
| 1459 | S1M10000003G10 | Staphylococcus aureus |
| 1460 | S1M10000004A04 | Staphylococcus aureus |
| 1461 | S1M10000004A06 | Staphylococcus aureus |
| 1462 | S1M10000004A07 | Staphylococcus aureus |
| 1463 | S1M10000004A11 | Staphylococcus aureus |
| 1464 | S1M10000004A12 | Staphylococcus aureus |
| 1465 | S1M10000004B03 | Staphylococcus aureus |
| 1466 | S1M10000004B04 | Staphylococcus aureus |
| 1467 | S1M10000004B06 | Staphylococcus aureus |
| 1468 | S1M10000004B08 | Staphylococcus aureus |
| 1469 | S1M10000004B09 | Staphylococcus aureus |
| 1470 | S1M10000004B11 | Staphylococcus aureus |
| 1471 | S1M10000004C01 | Staphylococcus aureus |
| 1472 | S1M10000004C02 | Staphylococcus aureus |
| 1473 | S1M10000004C03 | Staphylococcus aureus |
| 1474 | S1M10000004C06 | Staphylococcus aureus |
| 1475 | S1M10000004C07 | Staphylococcus aureus |
| 1476 | S1M10000004C08 | Staphylococcus aureus |
| 1477 | S1M10000004C09 | Staphylococcus aureus |
| 1478 | S1M10000004C10 | Staphylococcus aureus |
| 1479 | S1M10000004C12 | Staphylococcus aureus |
| 1480 | S1M10000004D01 | Staphylococcus aureus |
| 1481 | S1M10000004D03 | Staphylococcus aureus |
| 1482 | S1M10000004D04 | Staphylococcus aureus |
| 1483 | S1M10000004D06 | Staphylococcus aureus |
| 1484 | S1M10000004D07 | Staphylococcus aureus |
| 1485 | S1M10000004D08 | Staphylococcus aureus |
| 1486 | S1M10000004D10 | Staphylococcus aureus |
| 1487 | S1M10000004D12 | Staphylococcus aureus |
| 1488 | S1M10000004E03 | Staphylococcus aureus |
| 1489 | S1M10000004E04 | Staphylococcus aureus |
| 1490 | S1M10000004E06 | Staphylococcus aureus |
| 1491 | S1M10000004E07 | Staphylococcus aureus |
| 1492 | S1M10000004E11 | Staphylococcus aureus |
| 1493 | S1M10000004E12 | Staphylococcus aureus |
| 1494 | S1M10000004F01 | Staphylococcus aureus |
| 1495 | S1M10000004F02 | Staphylococcus aureus |
| 1496 | S1M10000004F06 | Staphylococcus aureus |
| 1497 | S1M10000004F07 | Staphylococcus aureus |
| 1498 | S1M10000004F08 | Staphylococcus aureus |
| 1499 | S1M10000004F09 | Staphylococcus aureus |
| 1500 | S1M10000004F12 | Staphylococcus aureus |
| 1501 | S1M10000004G01 | Staphylococcus aureus |
| 1502 | S1M10000004G02 | Staphylococcus aureus |
| 1503 | S1M10000004G03 | Staphylococcus aureus |
| 1504 | S1M10000004G05 | Staphylococcus aureus |
| 1505 | S1M10000004G06 | Staphylococcus aureus |
| 1506 | S1M10000004G07 | Staphylococcus aureus |
| 1507 | S1M10000004G09 | Staphylococcus aureus |
| 1508 | S1M10000004G12 | Staphylococcus aureus |
| 1509 | S1M10000005A01 | Staphylococcus aureus |
| 1510 | S1M10000005A03 | Staphylococcus aureus |
| 1511 | S1M10000005A05 | Staphylococcus aureus |
| 1512 | S1M10000005A06 | Staphylococcus aureus |
| 1513 | S1M10000005A07 | Staphylococcus aureus |
| 1514 | S1M10000005A08 | Staphylococcus aureus |
| 1515 | S1M10000005A09 | Staphylococcus aureus |
| 1516 | S1M10000005A10 | Staphylococcus aureus |
| 1517 | S1M10000005A11 | Staphylococcus aureus |
| 1518 | S1M10000005B02 | Staphylococcus aureus |
| 1519 | S1M10000005B04 | Staphylococcus aureus |
| 1520 | S1M10000005B07 | Staphylococcus aureus |
| 1521 | S1M10000005B08 | Staphylococcus aureus |
| 1522 | S1M10000005B09 | Staphylococcus aureus |
| 1523 | S1M10000005B12 | Staphylococcus aureus |
| 1524 | S1M10000005C01 | Staphylococcus aureus |
| 1525 | S1M10000005C05 | Staphylococcus aureus |
| 1526 | S1M10000005C06 | Staphylococcus aureus |
| 1527 | S1M10000005C09 | Staphylococcus aureus |
| 1528 | S1M10000005C11 | Staphylococcus aureus |
| 1529 | S1M10000005D01 | Staphylococcus aureus |
| 1530 | S1M10000005D02 | Staphylococcus aureus |
| 1531 | S1M10000005D03 | Staphylococcus aureus |
| 1532 | S1M10000005D04 | Staphylococcus aureus |
| 1533 | S1M10000005D05 | Staphylococcus aureus |
| 1534 | S1M10000005D06 | Staphylococcus aureus |
| 1535 | S1M10000005D07 | Staphylococcus aureus |
| 1536 | S1M10000005D08 | Staphylococcus aureus |
| 1537 | S1M10000005D09 | Staphylococcus aureus |
| 1538 | S1M10000005D11 | Staphylococcus aureus |
| 1539 | S1M10000005D12 | Staphylococcus aureus |
| 1540 | S1M10000005E01 | Staphylococcus aureus |
| 1541 | S1M10000005E02 | Staphylococcus aureus |
| 1542 | S1M10000005E05 | Staphylococcus aureus |
| 1543 | S1M10000005E06 | Staphylococcus aureus |
| 1544 | S1M10000005E07 | Staphylococcus aureus |
| 1545 | S1M10000005E08 | Staphylococcus aureus |
| 1546 | S1M10000005E10 | Staphylococcus aureus |
| 1547 | S1M10000005E11 | Staphylococcus aureus |
| 1548 | S1M10000005E12 | Staphylococcus aureus |
| 1549 | S1M10000005F02 | Staphylococcus aureus |
| 1550 | S1M10000005F03 | Staphylococcus aureus |
| 1551 | S1M10000005F04 | Staphylococcus aureus |
| 1552 | S1M10000006A03 | Staphylococcus aureus |
| 1553 | S1M10000006A04 | Staphylococcus aureus |
| 1554 | S1M10000006A05 | Staphylococcus aureus |
| 1555 | S1M10000006A07 | Staphylococcus aureus |
| 1556 | S1M10000006A08 | Staphylococcus aureus |
| 1557 | S1M10000006A10 | Staphylococcus aureus |
| 1558 | S1M10000006A12 | Staphylococcus aureus |
| 1559 | S1M10000006B02 | Staphylococcus aureus |
| 1560 | S1M10000006B03 | Staphylococcus aureus |
| 1561 | S1M10000006B04 | Staphylococcus aureus |
| 1562 | S1M10000006B07 | Staphylococcus aureus |
| 1563 | S1M10000006B10 | Staphylococcus aureus |
| 1564 | S1M10000006B11 | Staphylococcus aureus |
| 1565 | S1M10000006C02 | Staphylococcus aureus |
| 1566 | S1M10000006C04 | Staphylococcus aureus |
| 1567 | S1M10000006C06 | Staphylococcus aureus |
| 1568 | S1M10000006C07 | Staphylococcus aureus |
| 1569 | S1M10000006C08 | Staphylococcus aureus |
| 1570 | S1M10000006C10 | Staphylococcus aureus |
| 1571 | S1M10000006D03 | Staphylococcus aureus |
| 1572 | S1M10000006D05 | Staphylococcus aureus |
| 1573 | S1M10000006D06 | Staphylococcus aureus |
| 1574 | S1M10000006D07 | Staphylococcus aureus |
| 1575 | S1M10000006D08 | Staphylococcus aureus |
| 1576 | S1M10000006E02 | Staphylococcus aureus |
| 1577 | S1M10000006E03 | Staphylococcus aureus |
| 1578 | S1M10000006E04 | Staphylococcus aureus |
| 1579 | S1M10000006E07 | Staphylococcus aureus |
| 1580 | S1M10000006E08 | Staphylococcus aureus |
| 1581 | S1M10000006F01 | Staphylococcus aureus |
| 1582 | S1M10000006F02 | Staphylococcus aureus |
| 1583 | S1M10000006F03 | Staphylococcus aureus |
| 1584 | S1M10000006F04 | Staphylococcus aureus |
| 1585 | S1M10000006F06 | Staphylococcus aureus |
| 1586 | S1M10000006G02 | Staphylococcus aureus |
| 1587 | S1M10000006G03 | Staphylococcus aureus |
| 1588 | S1M10000006G05 | Staphylococcus aureus |
| 1589 | S1M10000006G06 | Staphylococcus aureus |
| 1590 | S1M10000006G07 | Staphylococcus aureus |
| 1591 | S1M10000006G09 | Staphylococcus aureus |
| 1592 | S1M10000006G10 | Staphylococcus aureus |
| 1593 | S1M10000006G11 | Staphylococcus aureus |
| 1594 | S1M10000007A02 | Staphylococcus aureus |
| 1595 | S1M10000007A03 | Staphylococcus aureus |
| 1596 | S1M10000007B02 | Staphylococcus aureus |
| 1597 | S1M10000007B11 | Staphylococcus aureus |
| 1598 | S1M10000007C02 | Staphylococcus aureus |
| 1599 | S1M10000007C04 | Staphylococcus aureus |
| 1600 | S1M10000007C05 | Staphylococcus aureus |
| 1601 | S1M10000007CO6 | Staphylococcus aureus |
| 1602 | S1M10000007C07 | Staphylococcus aureus |
| 1603 | S1M10000007C08 | Staphylococcus aureus |
| 1604 | S1M10000007C09 | Staphylococcus aureus |
| 1605 | S1M10000007D03 | Staphylococcus aureus |
| 1606 | S1M10000007D06 | Staphylococcus aureus |
| 1607 | S1M10000007D08 | Staphylococcus aureus |
| 1608 | S1M10000007D10 | Staphylococcus aureus |
| 1609 | S1M10000007D11 | Staphylococcus aureus |
| 1610 | S1M10000007E04 | Staphylococcus aureus |
| 1611 | S1M10000007E06 | Staphylococcus aureus |
| 1612 | S1M10000007E07 | Staphylococcus aureus |
| 1613 | S1M10000007F01 | Staphylococcus aureus |
| 1614 | S1M10000007F02 | Staphylococcus aureus |
| 1615 | S1M10000007F04 | Staphylococcus aureus |
| 1616 | S1M10000007F08 | Staphylococcus aureus |
| 1617 | S1M10000007F09 | Staphylococcus aureus |
| 1618 | S1M10000007F10 | Staphylococcus aureus |
| 1619 | S1M10000007F11 | Staphylococcus aureus |
| 1620 | S1M10000007F12 | Staphylococcus aureus |
| 1621 | S1M10000007G02 | Staphylococcus aureus |
| 1622 | S1M10000007G03 | Staphylococcus aureus |
| 1623 | S1M10000007G05 | Staphylococcus aureus |
| 1624 | S1M10000007G07 | Staphylococcus aureus |
| 1625 | S1M10000007G08 | Staphylococcus aureus |
| 1626 | S1M10000008A03 | Staphylococcus aureus |
| 1627 | S1M10000008A04 | Staphylococcus aureus |
| 1628 | S1M10000008A05 | Staphylococcus aureus |
| 1629 | S1M10000008A08 | Staphylococcus aureus |
| 1630 | S1M10000008A09 | Staphylococcus aureus |
| 1631 | S1M10000008A12 | Staphylococcus aureus |
| 1632 | S1M10000008B03 | Staphylococcus aureus |
| 1633 | S1M10000008B04 | Staphylococcus aureus |
| 1634 | S1M10000008B06 | Staphylococcus aureus |
| 1635 | S1M10000008B08 | Staphylococcus aureus |
| 1636 | S1M10000008B09 | Staphylococcus aureus |
| 1637 | S1M10000008B10 | Staphylococcus aureus |
| 1638 | S1M10000008C05 | Staphylococcus aureus |
| 1639 | S1M10000008C06 | Staphylococcus aureus |
| 1640 | S1M10000008C07 | Staphylococcus aureus |
| 1641 | S1M10000008C08 | Staphylococcus aureus |
| 1642 | S1M10000008C09 | Staphylococcus aureus |
| 1643 | S1M10000008D05 | Staphylococcus aureus |
| 1644 | S1M10000008D09 | Staphylococcus aureus |
| 1645 | S1M10000008E05 | Staphylococcus aureus |
| 1646 | S1M10000008E08 | Staphylococcus aureus |
| 1647 | S1M10000008E09 | Staphylococcus aureus |
| 1648 | S1M10000008E10 | Staphylococcus aureus |
| 1649 | S1M10000008F01 | Staphylococcus aureus |
| 1650 | S1M10000008F02 | Staphylococcus aureus |
| 1651 | S1M10000008F03 | Staphylococcus aureus |
| 1652 | S1M10000008F06 | Staphylococcus aureus |
| 1653 | S1M10000008F08 | Staphylococcus aureus |
| 1654 | S1M10000008F09 | Staphylococcus aureus |
| 1655 | S1M10000008F10 | Staphylococcus aureus |
| 1656 | S1M10000008F11 | Staphylococcus aureus |
| 1657 | S1M10000008G02 | Staphylococcus aureus |
| 1658 | S1M10000008G03 | Staphylococcus aureus |
| 1659 | S1M10000008G05 | Staphylococcus aureus |
| 1660 | S1M10000009A02 | Staphylococcus aureus |
| 1661 | S1M10000009A04 | Staphylococcus aureus |
| 1662 | S1M10000009A07 | Staphylococcus aureus |
| 1663 | S1M10000009A08 | Staphylococcus aureus |
| 1664 | S1M10000009A09 | Staphylococcus aureus |
| 1665 | S1M10000009A10 | Staphylococcus aureus |
| 1666 | S1M10000009A11 | Staphylococcus aureus |
| 1667 | S1M10000009B01 | Staphylococcus aureus |
| 1668 | S1M10000009B02 | Staphylococcus aureus |
| 1669 | S1M10000009B03 | Staphylococcus aureus |
| 1670 | S1M10000009B04 | Staphylococcus aureus |
| 1671 | S1M10000009B05 | Staphylococcus aureus |
| 1672 | S1M10000009B06 | Staphylococcus aureus |
| 1673 | S1M10000009B07 | Staphylococcus aureus |
| 1674 | S1M10000009B10 | Staphylococcus aureus |
| 1675 | S1M10000009B11 | Staphylococcus aureus |
| 1676 | S1M10000009B12 | Staphylococcus aureus |
| 1677 | S1M10000009C01 | Staphylococcus aureus |
| 1678 | S1M10000009C02 | Staphylococcus aureus |
| 1679 | S1M10000009C05 | Staphylococcus aureus |
| 1680 | S1M10000009C06 | Staphylococcus aureus |
| 1681 | S1M10000009C07 | Staphylococcus aureus |
| 1682 | S1M10000009C08 | Staphylococcus aureus |
| 1683 | S1M10000009C09 | Staphylococcus aureus |
| 1684 | S1M10000009C10 | Staphylococcus aureus |
| 1685 | S1M10000009C11 | Staphylococcus aureus |
| 1686 | S1M10000009D01 | Staphylococcus aureus |
| 1687 | S1M10000009D02 | Staphylococcus aureus |
| 1688 | S1M10000009D03 | Staphylococcus aureus |
| 1689 | S1M10000009D04 | Staphylococcus aureus |
| 1690 | S1M10000009D05 | Staphylococcus aureus |
| 1691 | S1M10000009D07 | Staphylococcus aureus |
| 1692 | S1M10000009D09 | Staphylococcus aureus |
| 1693 | S1M10000009D11 | Staphylococcus aureus |
| 1694 | S1M10000009E02 | Staphylococcus aureus |
| 1695 | S1M10000009E06 | Staphylococcus aureus |
| 1696 | S1M10000009E08 | Staphylococcus aureus |
| 1697 | S1M10000009B09 | Staphylococcus aureus |
| 1698 | S1M10000009E11 | Staphylococcus aureus |
| 1699 | S1M10000009E12 | Staphylococcus aureus |
| 1700 | S1M10000009F01 | Staphylococcus aureus |
| 1701 | S1M10000009F02 | Staphylococcus aureus |
| 1702 | S1M10000009F03 | Staphylococcus aureus |
| 1703 | S1M10000009F05 | Staphylococcus aureus |
| 1704 | S1M10000009F06 | Staphylococcus aureus |
| 1705 | S1M10000009F07 | Staphylococcus aureus |
| 1706 | S1M10000009F09 | Staphylococcus aureus |
| 1707 | S1M10000009F10 | Staphylococcus aureus |
| 1708 | S1M10000009G02 | Staphylococcus aureus |
| 1709 | S1M10000009G03 | Staphylococcus aureus |
| 1710 | S1M10000009G05 | Staphylococcus aureus |
| 1711 | S1M10000009G06 | Staphylococcus aureus |
| 1712 | S1M10000009G07 | Staphylococcus aureus |
| 1713 | S1M10000009G09 | Staphylococcus aureus |
| 1714 | S1M10000009G10 | Staphylococcus aureus |
| 1715 | S1M10000009G11 | Staphylococcus aureus |
| 1716 | S1M10000009H01 | Staphylococcus aureus |
| 1717 | S1M10000009H02 | Staphylococcus aureus |
| 1718 | S1M10000009H03 | Staphylococcus aureus |
| 1719 | S1M10000009H05 | Staphylococcus aureus |
| 1720 | S1M10000009H07 | Staphylococcus aureus |
| 1721 | S1M10000009H09 | Staphylococcus aureus |
| 1722 | S1M10000009H11 | Staphylococcus aureus |
| 1723 | S1M10000011A02 | Staphylococcus aureus |
| 1724 | S1M10000011A03 | Staphylococcus aureus |
| 1725 | S1M10000011A04 | Staphylococcus aureus |
| 1726 | S1M10000011A06 | Staphylococcus aureus |
| 1727 | S1M10000011B01 | Staphylococcus aureus |
| 1728 | S1M10000011B02 | Staphylococcus aureus |
| 1729 | S1M10000011B03 | Staphylococcus aureus |
| 1730 | S1M10000011B04 | Staphylococcus aureus |
| 1731 | S1M10000011B05 | Staphylococcus aureus |
| 1732 | S1M10000011C01 | Staphylococcus aureus |
| 1733 | S1M10000011C05 | Staphylococcus aureus |
| 1734 | S1M10000011C06 | Staphylococcus aureus |
| 1735 | S1M10000011D01 | Staphylococcus aureus |
| 1736 | S1M10000011D02 | Staphylococcus aureus |
| 1737 | S1M10000011D04 | Staphylococcus aureus |
| 1738 | S1M10000011D06 | Staphylococcus aureus |
| 1739 | S1M10000011E02 | Staphylococcus aureus |
| 1740 | S1M10000011E03 | Staphylococcus aureus |
| 1741 | S1M10000011E04 | Staphylococcus aureus |
| 1742 | S1M10000011F01 | Staphylococcus aureus |
| 1743 | S1M10000011F03 | Staphylococcus aureus |
| 1744 | S1M10000011F04 | Staphylococcus aureus |
| 1745 | S1M10000011F06 | Staphylococcus aureus |
| 1746 | S1M10000011G01 | Staphylococcus aureus |
| 1747 | S1M10000011G03 | Staphylococcus aureus |
| 1748 | S1M10000011G04 | Staphylococcus aureus |
| 1749 | S1M10000011G05 | Staphylococcus aureus |
| 1750 | S1M10000011G06 | Staphylococcus aureus |
| 1751 | S1M10000011H01 | Staphylococcus aureus |
| 1752 | S1M10000011H03 | Staphylococcus aureus |
| 1753 | S1M10000011H04 | Staphylococcus aureus |
| 1754 | S1M10000012A02 | Staphylococcus aureus |
| 1755 | S1M10000012A06 | Staphylococcus aureus |
| 1756 | S1M10000012A08 | Staphylococcus aureus |
| 1757 | S1M10000012A09 | Staphylococcus aureus |
| 1758 | S1M10000012A10 | Staphylococcus aureus |
| 1759 | S1M10000012A11 | Staphylococcus aureus |
| 1760 | S1M10000012B01 | Staphylococcus aureus |
| 1761 | S1M10000012B05 | Staphylococcus aureus |
| 1762 | S1M10000012B06 | Staphylococcus aureus |
| 1763 | S1M10000012B07 | Staphylococcus aureus |
| 1764 | S1M10000012B11 | Staphylococcus aureus |
| 1765 | S1M10000012C01 | Staphylococcus aureus |
| 1766 | S1M10000012C03 | Staphylococcus aureus |
| 1767 | S1M10000012C04 | Staphylococcus aureus |
| 1768 | S1M10000012C05 | Staphylococcus aureus |
| 1769 | S1M10000012C06 | Staphylococcus aureus |
| 1770 | S1M10000012C11 | Staphylococcus aureus |
| 1771 | S1M10000012C12 | Staphylococcus aureus |
| 1772 | S1M10000012D04 | Staphylococcus aureus |
| 1773 | S1M10000012D06 | Staphylococcus aureus |
| 1774 | S1M10000012D07 | Staphylococcus aureus |
| 1775 | S1M10000012D08 | Staphylococcus aureus |
| 1776 | S1M10000012D09 | Staphylococcus aureus |
| 1777 | S1M10000012D12 | Staphylococcus aureus |
| 1778 | S1M10000012E01 | Staphylococcus aureus |
| 1779 | S1M10000012E02 | Staphylococcus aureus |
| 1780 | S1M10000012E04 | Staphylococcus aureus |
| 1781 | S1M10000012E07 | Staphylococcus aureus |
| 1782 | S1M10000012E08 | Staphylococcus aureus |
| 1783 | S1M10000012E12 | Staphylococcus aureus |
| 1784 | S1M10000012F04 | Staphylococcus aureus |
| 1785 | S1M10000012F07 | Staphylococcus aureus |
| 1786 | S1M10000012F08 | Staphylococcus aureus |
| 1787 | S1M10000012F09 | Staphylococcus aureus |
| 1788 | S1M10000012F10 | Staphylococcus aureus |
| 1789 | S1M10000012F11 | Staphylococcus aureus |
| 1790 | S1M10000012F12 | Staphylococcus aureus |
| 1791 | S1M10000012G01 | Staphylococcus aureus |
| 1792 | S1M10000012G02 | Staphylococcus aureus |
| 1793 | S1M10000012G03 | Staphylococcus aureus |
| 1794 | S1M10000012G06 | Staphylococcus aureus |
| 1795 | S1M10000012G07 | Staphylococcus aureus |
| 1796 | S1M10000012G08 | Staphylococcus aureus |
| 1797 | S1M10000012G10 | Staphylococcus aureus |
| 1798 | S1M10000012H05 | Staphylococcus aureus |
| 1799 | S1M10000012H08 | Staphylococcus aureus |
| 1800 | S1M10000012H09 | Staphylococcus aureus |
| 1801 | S1M10000012H10 | Staphylococcus aureus |
| 1802 | S1M10000012H11 | Staphylococcus aureus |
| 1803 | S1M10000013A02 | Staphylococcus aureus |
| 1804 | S1M10000013A03 | Staphylococcus aureus |
| 1805 | S1M10000013A05 | Staphylococcus aureus |
| 1806 | S1M10000013A07 | Staphylococcus aureus |
| 1807 | S1M10000013A08 | Staphylococcus aureus |
| 1808 | S1M10000013A09 | Staphylococcus aureus |
| 1809 | S1M10000013A10 | Staphylococcus aureus |
| 1810 | S1M10000013A11 | Staphylococcus aureus |
| 1811 | S1M10000013A12 | Staphylococcus aureus |
| 1812 | S1M10000013B02 | Staphylococcus aureus |
| 1813 | S1M10000013B03 | Staphylococcus aureus |
| 1814 | S1M10000013B04 | Staphylococcus aureus |
| 1815 | S1M10000013B05 | Staphylococcus aureus |
| 1816 | S1M10000013B06 | Staphylococcus aureus |
| 1817 | S1M10000013B07 | Staphylococcus aureus |
| 1818 | S1M10000013B09 | Staphylococcus aureus |
| 1819 | S1M10000013B11 | Staphylococcus aureus |
| 1820 | S1M10000013C03 | Staphylococcus aureus |
| 1821 | S1M10000013C05 | Staphylococcus aureus |
| 1822 | S1M10000013C07 | Staphylococcus aureus |
| 1823 | S1M10000013C08 | Staphylococcus aureus |
| 1824 | S1M10000013C09 | Staphylococcus aureus |
| 1825 | S1M10000013C10 | Staphylococcus aureus |
| 1826 | S1M10000013C11 | Staphylococcus aureus |
| 1827 | S1M10000013C12 | Staphylococcus aureus |
| 1828 | S1M10000013D08 | Staphylococcus aureus |
| 1829 | S1M10000013D09 | Staphylococcus aureus |
| 1830 | S1M10000013D11 | Staphylococcus aureus |
| 1831 | S1M10000013E01 | Staphylococcus aureus |
| 1832 | S1M10000013E02 | Staphylococcus aureus |
| 1833 | S1M10000013E04 | Staphylococcus aureus |
| 1834 | S1M10000013E06 | Staphylococcus aureus |
| 1835 | S1M10000013E08 | Staphylococcus aureus |
| 1836 | S1M10000013E09 | Staphylococcus aureus |
| 1837 | S1M10000013E10 | Staphylococcus aureus |
| 1838 | S1M10000013F02 | Staphylococcus aureus |
| 1839 | S1M10000013F03 | Staphylococcus aureus |
| 1840 | S1M10000013F06 | Staphylococcus aureus |
| 1841 | S1M10000013F07 | Staphylococcus aureus |
| 1842 | S1M10000013F08 | Staphylococcus aureus |
| 1843 | S1M10000013F09 | Staphylococcus aureus |
| 1844 | S1M10000013F12 | Staphylococcus aureus |
| 1845 | S1M10000013G01 | Staphylococcus aureus |
| 1846 | S1M10000013G04 | Staphylococcus aureus |
| 1847 | S1M10000013G05 | Staphylococcus aureus |
| 1848 | S1M10000013G06 | Staphylococcus aureus |
| 1849 | S1M10000013G07 | Staphylococcus aureus |
| 1850 | S1M10000013G10 | Staphylococcus aureus |
| 1851 | S1M10000013G11 | Staphylococcus aureus |
| 1852 | S1M10000013G12 | Staphylococcus aureus |
| 1853 | S1M10000013H03 | Staphylococcus aureus |
| 1854 | S1M10000013H04 | Staphylococcus aureus |
| 1855 | S1M10000013H05 | Staphylococcus aureus |
| 1856 | S1M10000013H07 | Staphylococcus aureus |
| 1857 | S1M10000013H09 | Staphylococcus aureus |
| 1858 | S1M10000013H10 | Staphylococcus aureus |
| 1859 | S1M10000013H11 | Staphylococcus aureus |
| 1860 | S1M10000014A02 | Staphylococcus aureus |
| 1861 | S1M10000014A03 | Staphylococcus aureus |
| 1862 | S1M10000014A05 | Staphylococcus aureus |
| 1863 | S1M10000014A07 | Staphylococcus aureus |
| 1864 | S1M10000014A08 | Staphylococcus aureus |
| 1865 | S1M10000014A11 | Staphylococcus aureus |
| 1866 | S1M10000014A12 | Staphylococcus aureus |
| 1867 | S1M10000014B01 | Staphylococcus aureus |
| 1868 | S1M10000014B02 | Staphylococcus aureus |
| 1869 | S1M10000014B03 | Staphylococcus aureus |
| 1870 | S1M10000014B04 | Staphylococcus aureus |
| 1871 | S1M10000014B05 | Staphylococcus aureus |
| 1872 | S1M10000014B06 | Staphylococcus aureus |
| 1873 | S1M10000014B07 | Staphylococcus aureus |
| 1874 | S1M10000014B08 | Staphylococcus aureus |
| 1875 | S1M10000014B10 | Staphylococcus aureus |
| 1876 | S1M10000014B11 | Staphylococcus aureus |
| 1877 | S1M10000014B12 | Staphylococcus aureus |
| 1878 | S1M10000014C01 | Staphylococcus aureus |
| 1879 | S1M10000014C05 | Staphylococcus aureus |
| 1880 | S1M10000014C06 | Staphylococcus aureus |
| 1881 | S1M10000014C07 | Staphylococcus aureus |
| 1882 | S1M10000014C09 | Staphylococcus aureus |
| 1883 | S1M10000014C10 | Staphylococcus aureus |
| 1884 | S1M10000014C11 | Staphylococcus aureus |
| 1885 | S1M10000014C12 | Staphylococcus aureus |
| 1886 | S1M10000014D03 | Staphylococcus aureus |
| 1887 | S1M10000014D06 | Staphylococcus aureus |
| 1888 | S1M10000014D08 | Staphylococcus aureus |
| 1889 | S1M10000014D09 | Staphylococcus aureus |
| 1890 | S1M10000014D10 | Staphylococcus aureus |
| 1891 | S1M10000014E01 | Staphylococcus aureus |
| 1892 | S1M10000014E04 | Staphylococcus aureus |
| 1893 | S1M10000014E05 | Staphylococcus aureus |
| 1894 | S1M10000014E07 | Staphylococcus aureus |
| 1895 | S1M10000014E08 | Staphylococcus aureus |
| 1896 | S1M10000014E09 | Staphylococcus aureus |
| 1897 | S1M10000014E10 | Staphylococcus aureus |
| 1898 | S1M10000014E12 | Staphylococcus aureus |
| 1899 | S1M10000014F02 | Staphylococcus aureus |
| 1900 | S1M10000014F03 | Staphylococcus aureus |
| 1901 | S1M10000014F04 | Staphylococcus aureus |
| 1902 | S1M10000014F05 | Staphylococcus aureus |
| 1903 | S1M10000014F08 | Staphylococcus aureus |
| 1904 | S1M10000014F09 | Staphylococcus aureus |
| 1905 | S1M10000014F10 | Staphylococcus aureus |
| 1906 | S1M10000014G02 | Staphylococcus aureus |
| 1907 | S1M10000014G04 | Staphylococcus aureus |
| 1908 | S1M10000014G06 | Staphylococcus aureus |
| 1909 | S1M10000014G07 | Staphylococcus aureus |
| 1910 | S1M10000014G08 | Staphylococcus aureus |
| 1911 | S1M10000014G12 | Staphylococcus aureus |
| 1912 | S1M10000014H02 | Staphylococcus aureus |
| 1913 | S1M10000014H03 | Staphylococcus aureus |
| 1914 | S1M10000014H04 | Staphylococcus aureus |
| 1915 | S1M10000014H05 | Staphylococcus aureus |
| 1916 | S1M10000014H06 | Staphylococcus aureus |
| 1917 | S1M10000014H07 | Staphylococcus aureus |
| 1918 | S1M10000014HO8 | Staphylococcus aureus |
| 1919 | S1M10000014H11 | Staphylococcus aureus |
| 1920 | S1M10000015A02 | Staphylococcus aureus |
| 1921 | S1M10000015A03 | Staphylococcus aureus |
| 1922 | S1M10000015A05 | Staphylococcus aureus |
| 1923 | S1M10000015A06 | Staphylococcus aureus |
| 1924 | S1M10000015A09 | Staphylococcus aureus |
| 1925 | S1M10000015A10 | Staphylococcus aureus |
| 1926 | S1M10000015A11 | Staphylococcus aureus |
| 1927 | S1M10000015A12 | Staphylococcus aureus |
| 1928 | S1M10000015B02 | Staphylococcus aureus |
| 1929 | S1M10000015B05 | Staphylococcus aureus |
| 1930 | S1M10000015B08 | Staphylococcus aureus |
| 1931 | S1M10000015B09 | Staphylococcus aureus |
| 1932 | S1M10000015B10 | Staphylococcus aureus |
| 1933 | S1M10000015C01 | Staphylococcus aureus |
| 1934 | S1M10000015C02 | Staphylococcus aureus |
| 1935 | S1M10000015C03 | Staphylococcus aureus |
| 1936 | S1M10000015C05 | Staphylococcus aureus |
| 1937 | S1M10000015C06 | Staphylococcus aureus |
| 1938 | S1M10000015C08 | Staphylococcus aureus |
| 1939 | S1M10000015C10 | Staphylococcus aureus |
| 1940 | S1M10000015C12 | Staphylococcus aureus |
| 1941 | S1M10000015D02 | Staphylococcus aureus |
| 1942 | S1M10000015D03 | Staphylococcus aureus |
| 1943 | S1M10000015D04 | Staphylococcus aureus |
| 1944 | S1M10000015D05 | Staphylococcus aureus |
| 1945 | S1M10000015D06 | Staphylococcus aureus |
| 1946 | S1M10000015D12 | Staphylococcus aureus |
| 1947 | S1M10000015E02 | Staphylococcus aureus |
| 1948 | S1M10000015E03 | Staphylococcus aureus |
| 1949 | S1M10000015E06 | Staphylococcus aureus |
| 1950 | S1M10000015E07 | Staphylococcus aureus |
| 1951 | S1M10000015E09 | Staphylococcus aureus |
| 1952 | S1M10000015E10 | Staphylococcus aureus |
| 1953 | S1M10000015E11 | Staphylococcus aureus |
| 1954 | S1M10000015E12 | Staphylococcus aureus |
| 1955 | S1M10000015F01 | Staphylococcus aureus |
| 1956 | S1M10000015F02 | Staphylococcus aureus |
| 1957 | S1M10000015F03 | Staphylococcus aureus |
| 1958 | SIM10000015F04 | Staphylococcus aureus |
| 1959 | S1M10000015F06 | Staphylococcus aureus |
| 1960 | S1M10000015F07 | Staphylococcus aureus |
| 1961 | S1M10000015F08 | Staphylococcus aureus |
| 1962 | S1M10000015F09 | Staphylococcus aureus |
| 1963 | S1M10000015F10 | Staphylococcus aureus |
| 1964 | S1M10000015G01 | Staphylococcus aureus |
| 1965 | S1M10000015G02 | Staphylococcus aureus |
| 1966 | S1M10000015G03 | Staphylococcus aureus |
| 1967 | S1M10000015G04 | Staphylococcus aureus |
| 1968 | S1M10000015G05 | Staphylococcus aureus |
| 1969 | S1M10000015G06 | Staphylococcus aureus |
| 1970 | S1M10000015G07 | Staphylococcus aureus |
| 1971 | S1M10000015G08 | Staphylococcus aureus |
| 1972 | SIM10000015G09 | Staphylococcus aureus |
| 1973 | S1M10000015G10 | Staphylococcus aureus |
| 1974 | S1M10000015G11 | Staphylococcus aureus |
| 1975 | S1M10000015H04 | Staphylococcus aureus |
| 1976 | S1M10000015H06 | Staphylococcus aureus |
| 1977 | S1M10000016A03 | Staphylococcus aureus |
| 1978 | S1M10000016A04 | Staphylococcus aureus |
| 1979 | S1M10000016A06 | Staphylococcus aureus |
| 1980 | S1M10000016A07 | Staphylococcus aureus |
| 1981 | S1M10000016A09 | Staphylococcus aureus |
| 1982 | S1M10000016A10 | Staphylococcus aureus |
| 1983 | S1M10000016A12 | Staphylococcus aureus |
| 1984 | S1M10000016B02 | Staphylococcus aureus |
| 1985 | S1M10000016B05 | Staphylococcus aureus |
| 1986 | S1M10000016B06 | Staphylococcus aureus |
| 1987 | S1M10000016B07 | Staphylococcus aureus |
| 1988 | S1M10000016B08 | Staphylococcus aureus |
| 1989 | S1M10000016B09 | Staphylococcus aureus |
| 1990 | S1M10000016B10 | Staphylococcus aureus |
| 1991 | S1M10000016B11 | Staphylococcus aureus |
| 1992 | S1M10000016B12 | Staphylococcus aureus |
| 1993 | S1M10000016C01 | Staphylococcus aureus |
| 1994 | S1M10000016C02 | Staphylococcus aureus |
| 1995 | S1M10000016C04 | Staphylococcus aureus |
| 1996 | S1M10000016C05 | Staphylococcus aureus |
| 1997 | S1M10000016C06 | Staphylococcus aureus |
| 1998 | S1M10000016C08 | Staphylococcus aureus |
| 1999 | S1M10000016C09 | Staphylococcus aureus |
| 2000 | S1M10000016C10 | Staphylococcus aureus |
| 2001 | S1M10000016C11 | Staphylococcus aureus |
| 2002 | S1M10000016C12 | Staphylococcus aureus |
| 2003 | S1M10000016D01 | Staphylococcus aureus |
| 2004 | S1M10000016D02 | Staphylococcus aureus |
| 2005 | S1M10000016D04 | Staphylococcus aureus |
| 2006 | S1M10000016D05 | Staphylococcus aureus |
| 2007 | S1M10000016D06 | Staphylococcus aureus |
| 2008 | S1M10000016D08 | Staphylococcus aureus |
| 2009 | S1M10000016D09 | Staphylococcus aureus |
| 2010 | S1M10000016D10 | Staphylococcus aureus |
| 2011 | S1M10000016D11 | Staphylococcus aureus |
| 2012 | S1M10000016E04 | Staphylococcus aureus |
| 2013 | S1M10000016E05 | Staphylococcus aureus |
| 2014 | S1M10000016E06 | Staphylococcus aureus |
| 2015 | S1M10000016E07 | Staphylococcus aureus |
| 2016 | S1M10000016E08 | Staphylococcus aureus |
| 2017 | S1M10000016E09 | Staphylococcus aureus |
| 2018 | S1M10000016E10 | Staphylococcus aureus |
| 2019 | S1M10000016E11 | Staphylococcus aureus |
| 2020 | S1M10000016E12 | Staphylococcus aureus |
| 2021 | S1M10000016F02 | Staphylococcus aureus |
| 2022 | S1M10000016F03 | Staphylococcus aureus |
| 2023 | S1M10000016F05 | Staphylococcus aureus |
| 2024 | S1M10000016F06 | Staphylococcus aureus |
| 2025 | S1M10000016F08 | Staphylococcus aureus |
| 2026 | S1M10000016F09 | Staphylococcus aureus |
| 2027 | S1M10000016F11 | Staphylococcus aureus |
| 2028 | S1M10000016G01 | Staphylococcus aureus |
| 2029 | S1M10000016G03 | Staphylococcus aureus |
| 2030 | S1M10000016G04 | Staphylococcus aureus |
| 2031 | S1M10000016G05 | Staphylococcus aureus |
| 2032 | S1M10000016H03 | Staphylococcus aureus |
| 2033 | S1M10000016H04 | Staphylococcus aureus |
| 2034 | S1M10000016H08 | Staphylococcus aureus |
| 2035 | S1M10000016H10 | Staphylococcus aureus |
| 2036 | S1M10000017A02 | Staphylococcus aureus |
| 2037 | S1M10000017A03 | Staphylococcus aureus |
| 2038 | S1M10000017A04 | Staphylococcus aureus |
| 2039 | S1M10000017A08 | Staphylococcus aureus |
| 2040 | S1M1OOOOO17A11 | Staphylococcus aureus |
| 2041 | S1M10000017A12 | Staphylococcus aureus |
| 2042 | S1M10000017B02 | Staphylococcus aureus |
| 2043 | S1M10000017B05 | Staphylococcus aureus |
| 2044 | S1M10000017B07 | Staphylococcus aureus |
| 2045 | S1M10000017B08 | Staphylococcus aureus |
| 2046 | S1M10000017B09 | Staphylococcus aureus |
| 2047 | S1M10000017B10 | Staphylococcus aureus |
| 2048 | S1M10000017B11 | Staphylococcus aureus |
| 2049 | S1M10000017B12 | Staphylococcus aureus |
| 2050 | S1M10000017C01 | Staphylococcus aureus |
| 2051 | S1M10000017C03 | Staphylococcus aureus |
| 2052 | S1M10000017C05 | Staphylococcus aureus |
| 2053 | S1M10000017C08 | Staphylococcus aureus |
| 2054 | S1M10000017C09 | Staphylococcus aureus |
| 2055 | S1M10000017C10 | Staphylococcus aureus |
| 2056 | S1M10000017C11 | Staphylococcus aureus |
| 2057 | S1M10000017C12 | Staphylococcus aureus |
| 2058 | S1M10000017D03 | Staphylococcus aureus |
| 2059 | S1M10000017D09 | Staphylococcus aureus |
| 2060 | S1M10000017D10 | Staphylococcus aureus |
| 2061 | S1M10000017E04 | Staphylococcus aureus |
| 2062 | S1M10000017E05 | Staphylococcus aureus |
| 2063 | S1M10000017E08 | Staphylococcus aureus |
| 2064 | S1M10000017E11 | Staphylococcus aureus |
| 2065 | S1M10000017F01 | Staphylococcus aureus |
| 2066 | S1M10000017FO4 | Staphylococcus aureus |
| 2067 | S1M10000017F05 | Staphylococcus aureus |
| 2068 | S1M10000017F06 | Staphylococcus aureus |
| 2069 | S1M10000017F11 | Staphylococcus aureus |
| 2070 | S1M10000017G02 | Staphylococcus aureus |
| 2071 | S1M10000017G05 | Staphylococcus aureus |
| 2072 | S1M10000017G06 | Staphylococcus aureus |
| 2073 | S1M10000018A03 | Staphylococcus aureus |
| 2074 | S1M10000018A04 | Staphylococcus aureus |
| 2075 | S1M10000018A05 | Staphylococcus aureus |
| 2076 | S1M10000018A06 | Staphylococcus aureus |
| 2077 | S1M10000018A08 | Staphylococcus aureus |
| 2078 | S1M10000018A09 | Staphylococcus aureus |
| 2079 | S1M10000018A10 | Staphylococcus aureus |
| 2080 | S1M10000018A11 | Staphylococcus aureus |
| 2081 | S1M10000018B02 | Staphylococcus aureus |
| 2082 | S1M10000018BO3 | Staphylococcus aureus |
| 2083 | S1M10000018B05 | Staphylococcus aureus |
| 2084 | S1M10000018B09 | Staphylococcus aureus |
| 2085 | S1M10000018B10 | Staphylococcus aureus |
| 2086 | S1M10000018B11 | Staphylococcus aureus |
| 2087 | S1M10000018C01 | Staphylococcus aureus |
| 2088 | S1M10000018C02 | Staphylococcus aureus |
| 2089 | S1M10000018C03 | Staphylococcus aureus |
| 2090 | S1M10000018C04 | Staphylococcus aureus |
| 2091 | S1M10000018C05 | Staphylococcus aureus |
| 2092 | S1M10000018C06 | Staphylococcus aureus |
| 2093 | S1M10000018C08 | Staphylococcus aureus |
| 2094 | S1M10000018C09 | Staphylococcus aureus |
| 2095 | S1M10000018C10 | Staphylococcus aureus |
| 2096 | S1M10000018C11 | Staphylococcus aureus |
| 2097 | S1M10000018C12 | Staphylococcus aureus |
| 2098 | S1M10000018D01 | Staphylococcus aureus |
| 2099 | S1M10000018D02 | Staphylococcus aureus |
| 2100 | S1M10000018D03 | Staphylococcus aureus |
| 2101 | S1M10000018D04 | Staphylococcus aureus |
| 2102 | S1M10000018D09 | Staphylococcus aureus |
| 2103 | S1M10000018D10 | Staphylococcus aureus |
| 2104 | S1M10000018D11 | Staphylococcus aureus |
| 2105 | S1M10000018D12 | Staphylococcus aureus |
| 2106 | S1M10000018E01 | Staphylococcus aureus |
| 2107 | S1M10000018E02 | Staphylococcus aureus |
| 2108 | S1M10000018E03 | Staphylococcus aureus |
| 2109 | S1M10000018E04 | Staphylococcus aureus |
| 2110 | S1M10000018E05 | Staphylococcus aureus |
| 2111 | S1M10000018E08 | Staphylococcus aureus |
| 2112 | S1M10000018E09 | Staphylococcus aureus |
| 2113 | S1M10000018E11 | Staphylococcus aureus |
| 2114 | S1M10000018E12 | Staphylococcus aureus |
| 2115 | S1M10000018F03 | Staphylococcus aureus |
| 2116 | S1M10000018F04 | Staphylococcus aureus |
| 2117 | S1M10000018F07 | Staphylococcus aureus |
| 2118 | S1M10000018F09 | Staphylococcus aureus |
| 2119 | SIM10000018F10 | Staphylococcus aureus |
| 2120 | S1M10000018F12 | Staphylococcus aureus |
| 2121 | S1M10000018G03 | Staphylococcus aureus |
| 2122 | S1M10000018G05 | Staphylococcus aureus |
| 2123 | S1M10000018G07 | Staphylococcus aureus |
| 2124 | S1M10000018G08 | Staphylococcus aureus |
| 2125 | S1M10000018G09 | Staphylococcus aureus |
| 2126 | S1M10000018G10 | Staphylococcus aureus |
| 2127 | S1M10000018G12 | Staphylococcus aureus |
| 2128 | S1M10000018H01 | Staphylococcus aureus |
| 2129 | S1M10000018H02 | Staphylococcus aureus |
| 2130 | S1M10000018H07 | Staphylococcus aureus |
| 2131 | S1M10000018H09 | Staphylococcus aureus |
| 2132 | S1M10000018H10 | Staphylococcus aureus |
| 2133 | S1M10000019A02 | Staphylococcus aureus |
| 2134 | S1M10000019A03 | Staphylococcus aureus |
| 2135 | S1M10000019A05 | Staphylococcus aureus |
| 2136 | S1M10000019A06 | Staphylococcus aureus |
| 2137 | S1M10000019A07 | Staphylococcus aureus |
| 2138 | S1M10000019A09 | Staphylococcus aureus |
| 2139 | S1M10000019A11 | Staphylococcus aureus |
| 2140 | S1M10000019A12 | Staphylococcus aureus |
| 2141 | S1M10000019B03 | Staphylococcus aureus |
| 2142 | S1M10000019B04 | Staphylococcus aureus |
| 2143 | S1M10000019B07 | Staphylococcus aureus |
| 2144 | S1M10000019B08 | Staphylococcus aureus |
| 2145 | S1M10000019B09 | Staphylococcus aureus |
| 2146 | S1M10000019B10 | Staphylococcus aureus |
| 2147 | S1M10000019B11 | Staphylococcus aureus |
| 2148 | S1M10000019B12 | Staphylococcus aureus |
| 2149 | S1M10000019C01 | Staphylococcus aureus |
| 2150 | S1M10000019C04 | Staphylococcus aureus |
| 2151 | S1M10000019C05 | Staphylococcus aureus |
| 2152 | S1M10000019C06 | Staphylococcus aureus |
| 2153 | S1M10000019C07 | Staphylococcus aureus |
| 2154 | S1M10000019C08 | Staphylococcus aureus |
| 2155 | S1M10000019C11 | Staphylococcus aureus |
| 2156 | S1M10000019C12 | Staphylococcus aureus |
| 2157 | S1M10000019D01 | Staphylococcus aureus |
| 2158 | S1M10000019D02 | Staphylococcus aureus |
| 2159 | S1M10000019D04 | Staphylococcus aureus |
| 2160 | S1M10000019D05 | Staphylococcus aureus |
| 2161 | S1M10000019D06 | Staphylococcus aureus |
| 2162 | S1M10000019D07 | Staphylococcus aureus |
| 2163 | S1M10000019D09 | Staphylococcus aureus |
| 2164 | S1M10000019D12 | Staphylococcus aureus |
| 2165 | S1M10000019E01 | Staphylococcus aureus |
| 2166 | S1M10000019E02 | Staphylococcus aureus |
| 2167 | S1M10000019E07 | Staphylococcus aureus |
| 2168 | S1M10000019F01 | Staphylococcus aureus |
| 2169 | S1M10000019F05 | Staphylococcus aureus |
| 2170 | S1M10000019F06 | Staphylococcus aureus |
| 2171 | S1M10000019F08 | Staphylococcus aureus |
| 2172 | S1M10000019F09 | Staphylococcus aureus |
| 2173 | S1M10000019F11 | Staphylococcus aureus |
| 2174 | S1M10000019G04 | Staphylococcus aureus |
| 2175 | S1M10000019G07 | Staphylococcus aureus |
| 2176 | S1M10000019G09 | Staphylococcus aureus |
| 2177 | S1M10000019G10 | Staphylococcus aureus |
| 2178 | S1M10000019G11 | Staphylococcus aureus |
| 2179 | S1M10000019H05 | Staphylococcus aureus |
| 2180 | S1M10000019H08 | Staphylococcus aureus |
| 2181 | S1M10000020A05 | Staphylococcus aureus |
| 2182 | S1M10000020A06 | Staphylococcus aureus |
| 2183 | S1M10000020A07 | Staphylococcus aureus |
| 2184 | S1M10000020A11 | Staphylococcus aureus |
| 2185 | S1M10000020A12 | Staphylococcus aureus |
| 2186 | S1M10000020B02 | Staphylococcus aureus |
| 2187 | S1M10000020B03 | Staphylococcus aureus |
| 2188 | S1M10000020B05 | Staphylococcus aureus |
| 2189 | S1M10000020B06 | Staphylococcus aureus |
| 2190 | S1M10000020B07 | Staphylococcus aureus |
| 2191 | S1M10000020B09 | Staphylococcus aureus |
| 2192 | S1M10000020B12 | Staphylococcus aureus |
| 2193 | S1M10000020C09 | Staphylococcus aureus |
| 2194 | S1M10000020C10 | Staphylococcus aureus |
| 2195 | S1M10000020C11 | Staphylococcus aureus |
| 2196 | S1M10000020D03 | Staphylococcus aureus |
| 2197 | S1M10000020D04 | Staphylococcus aureus |
| 2198 | S1M10000020D06 | Staphylococcus aureus |
| 2199 | S1M10000020D07 | Staphylococcus aureus |
| 2200 | S1M10000020D08 | Staphylococcus aureus |
| 2201 | S1M10000020D09 | Staphylococcus aureus |
| 2202 | S1M10000020D12 | Staphylococcus aureus |
| 2203 | S1M10000020E01 | Staphylococcus aureus |
| 2204 | S1M10000020E03 | Staphylococcus aureus |
| 2205 | S1M10000020E04 | Staphylococcus aureus |
| 2206 | S1M10000020E06 | Staphylococcus aureus |
| 2207 | S1M10000020E08 | Staphylococcus aureus |
| 2208 | S1M10000020E11 | Staphylococcus aureus |
| 2209 | S1M10000020E12 | Staphylococcus aureus |
| 2210 | S1M10000020F01 | Staphylococcus aureus |
| 2211 | S1M10000020F05 | Staphylococcus aureus |
| 2212 | S1M10000020F06 | Staphylococcus aureus |
| 2213 | 51M10000020F07 | Staphylococcus aureus |
| 2214 | S1M10000020F09 | Staphylococcus aureus |
| 2215 | S1M10000020F11 | Staphylococcus aureus |
| 2216 | S1M10000020F12 | Staphylococcus aureus |
| 2217 | S1M10000020G01 | Staphylococcus aureus |
| 2218 | S1M10000020G05 | Staphylococcus aureus |
| 2219 | S1M10000020G07 | Staphylococcus aureus |
| 2220 | S1M10000020G08 | Staphylococcus aureus |
| 2221 | S1M10000020G09 | Staphylococcus aureus |
| 2222 | S1M10000020G10 | Staphylococcus aureus |
| 2223 | S1M10000020G11 | Staphylococcus aureus |
| 2224 | S1M10000020G12 | Staphylococcus aureus |
| 2225 | S1M10000020H01 | Staphylococcus aureus |
| 2226 | S1M10000020H02 | Staphylococcus aureus |
| 2227 | S1M10000020H04 | Staphylococcus aureus |
| 2228 | S1M10000020H06 | Staphylococcus aureus |
| 2229 | S1M10000020H08 | Staphylococcus aureus |
| 2230 | S1M10000020H10 | Staphylococcus aureus |
| 2231 | S1M10000020H11 | Staphylococcus aureus |
| 2232 | S1M10000021A04 | Staphylococcus aureus |
| 2233 | S1M10000021A05 | Staphylococcus aureus |
| 2234 | S1M10000021A06 | Staphylococcus aureus |
| 2235 | S1M10000021A07 | Staphylococcus aureus |
| 2236 | S1M10000021A08 | Staphylococcus aureus |
| 2237 | S1M10000021A09 | Staphylococcus aureus |
| 2238 | S1M10000021A10 | Staphylococcus aureus |
| 2239 | S1M10000021B05 | Staphylococcus aureus |
| 2240 | S1M10000021B06 | Staphylococcus aureus |
| 2241 | S1M10000021B07 | Staphylococcus aureus |
| 2242 | S1M10000021B10 | Staphylococcus aureus |
| 2243 | S1M10000021C04 | Staphylococcus aureus |
| 2244 | S1M10000021C05 | Staphylococcus aureus |
| 2245 | S1M10000021C07 | Staphylococcus aureus |
| 2246 | S1M10000021C08 | Staphylococcus aureus |
| 2247 | S1M10000021C10 | Staphylococcus aureus |
| 2248 | S1M10000021C11 | Staphylococcus aureus |
| 2249 | S1M10000021C12 | Staphylococcus aureus |
| 2250 | S1M10000021D01 | Staphylococcus aureus |
| 2251 | S1M10000021D03 | Staphylococcus aureus |
| 2252 | S1M10000021D04 | Staphylococcus aureus |
| 2253 | S1M10000021D06 | Staphylococcus aureus |
| 2254 | S1M10000021D09 | Staphylococcus aureus |
| 2255 | S1M10000021D10 | Staphylococcus aureus |
| 2256 | S1M10000021E01 | Staphylococcus aureus |
| 2257 | S1M10000021E02 | Staphylococcus aureus |
| 2258 | S1M10000021E03 | Staphylococcus aureus |
| 2259 | S1M10000021E05 | Staphylococcus aureus |
| 2260 | S1M10000021E06 | Staphylococcus aureus |
| 2261 | S1M10000021E09 | Staphylococcus aureus |
| 2262 | S1M10000021E12 | Staphylococcus aureus |
| 2263 | S1M10000021F02 | Staphylococcus aureus |
| 2264 | S1M10000021F04 | Staphylococcus aureus |
| 2265 | S1M10000021F05 | Staphylococcus aureus |
| 2266 | S1M10000021F06 | Staphylococcus aureus |
| 2267 | S1M10000021F07 | Staphylococcus aureus |
| 2268 | S1M10000021F09 | Staphylococcus aureus |
| 2269 | S1M10000021F11 | Staphylococcus aureus |
| 2270 | S1M10000021G01 | Staphylococcus aureus |
| 2271 | S1M10000021G03 | Staphylococcus aureus |
| 2272 | S1M10000021G08 | Staphylococcus aureus |
| 2273 | S1M10000021H04 | Staphylococcus aureus |
| 2274 | S1M10000021H05 | Staphylococcus aureus |
| 2275 | S1M10000021H07 | Staphylococcus aureus |
| 2276 | S1M10000021H08 | Staphylococcus aureus |
| 2277 | S1M10000021H11 | Staphylococcus aureus |
| 2278 | S1M10000022A02 | Staphylococcus aureus |
| 2279 | S1M10000022A03 | Staphylococcus aureus |
| 2280 | S1M10000022A05 | Staphylococcus aureus |
| 2281 | S1M10000022A08 | Staphylococcus aureus |
| 2282 | S1M10000022A09 | Staphylococcus aureus |
| 2283 | S1M10000022A12 | Staphylococcus aureus |
| 2284 | S1M10000022B02 | Staphylococcus aureus |
| 2285 | S1M10000022B03 | Staphylococcus aureus |
| 2286 | S1M10000022B05 | Staphylococcus aureus |
| 2287 | S1M10000022B06 | Staphylococcus aureus |
| 2288 | S1M10000022B08 | Staphylococcus aureus |
| 2289 | S1M10000022B09 | Staphylococcus aureus |
| 2290 | S1M10000022B10 | Staphylococcus aureus |
| 2291 | S1M10000022B11 | Staphylococcus aureus |
| 2292 | S1M10000022B12 | Staphylococcus aureus |
| 2293 | S1M10000022C02 | Staphylococcus aureus |
| 2294 | S1M10000022C03 | Staphylococcus aureus |
| 2295 | S1M10000022C04 | Staphylococcus aureus |
| 2296 | S1M10000022C06 | Staphylococcus aureus |
| 2297 | S1M10000022C07 | Staphylococcus aureus |
| 2298 | S1M10000022C08 | Staphylococcus aureus |
| 2299 | S1M10000022C11 | Staphylococcus aureus |
| 2300 | S1M10000022D03 | Staphylococcus aureus |
| 2301 | S1M10000022D05 | Staphylococcus aureus |
| 2302 | S1M10000022D06 | Staphylococcus aureus |
| 2303 | S1M10000022D07 | Staphylococcus aureus |
| 2304 | S1M10000022D08 | Staphylococcus aureus |
| 2305 | S1M10000022D09 | Staphylococcus aureus |
| 2306 | S1M10000022D11 | Staphylococcus aureus |
| 2307 | S1M10000022E01 | Staphylococcus aureus |
| 2308 | S1M10000022E03 | Staphylococcus aureus |
| 2309 | S1M10000022E05 | Staphylococcus aureus |
| 2310 | S1M10000022E09 | Staphylococcus aureus |
| 2311 | S1M10000022F04 | Staphylococcus aureus |
| 2312 | S1M10000022F06 | Staphylococcus aureus |
| 2313 | S1M10000022F07 | Staphylococcus aureus |
| 2314 | S1M10000022F08 | Staphylococcus aureus |
| 2315 | S1M10000022F11 | Staphylococcus aureus |
| 2316 | S1M10000022G03 | Staphylococcus aureus |
| 2317 | S1M10000022G04 | Staphylococcus aureus |
| 2318 | S1M10000022G07 | Staphylococcus aureus |
| 2319 | S1M10000022G08 | Staphylococcus aureus |
| 2320 | S1M10000022G12 | Staphylococcus aureus |
| 2321 | S1M10000022H03 | Staphylococcus aureus |
| 2322 | S1M10000022H05 | Staphylococcus aureus |
| 2323 | S1M10000022H06 | Staphylococcus aureus |
| 2324 | S1M10000022H07 | Staphylococcus aureus |
| 2325 | S1M10000022H08 | Staphylococcus aureus |
| 2326 | S1M10000022H11 | Staphylococcus aureus |
| 2327 | S1M10000023A05 | Staphylococcus aureus |
| 2328 | S1M10000023A09 | Staphylococcus aureus |
| 2329 | S1M10000023A11 | Staphylococcus aureus |
| 2330 | S1M10000023A12 | Staphylococcus aureus |
| 2331 | S1M10000023B01 | Staphylococcus aureus |
| 2332 | S1M10000023B03 | Staphylococcus aureus |
| 2333 | S1M10000023B07 | Staphylococcus aureus |
| 2334 | S1M10000023B08 | Staphylococcus aureus |
| 2335 | S1M10000023B09 | Staphylococcus aureus |
| 2336 | S1M10000023B10 | Staphylococcus aureus |
| 2337 | S1M10000023B11 | Staphylococcus aureus |
| 2338 | S1M10000023B12 | Staphylococcus aureus |
| 2339 | S1M10000023C02 | Staphylococcus aureus |
| 2340 | S1M10000023C10 | Staphylococcus aureus |
| 2341 | S1M10000023C11 | Staphylococcus aureus |
| 2342 | S1M10000023C12 | Staphylococcus aureus |
| 2343 | S1M10000023D01 | Staphylococcus aureus |
| 2344 | S1M10000023D03 | Staphylococcus aureus |
| 2345 | S1M10000023D04 | Staphylococcus aureus |
| 2346 | S1M10000023D07 | Staphylococcus aureus |
| 2347 | S1M10000023D08 | Staphylococcus aureus |
| 2348 | S1M10000023D09 | Staphylococcus aureus |
| 2349 | S1M10000023D10 | Staphylococcus aureus |
| 2350 | S1M10000023D12 | Staphylococcus aureus |
| 2351 | S1M10000023E01 | Staphylococcus aureus |
| 2352 | S1M10000023E04 | Staphylococcus aureus |
| 2353 | S1M10000023E07 | Staphylococcus aureus |
| 2354 | S1M10000023E10 | Staphylococcus aureus |
| 2355 | S1M10000023E11 | Staphylococcus aureus |
| 2356 | S1M10000023F04 | Staphylococcus aureus |
| 2357 | S1M10000023F07 | Staphylococcus aureus |
| 2358 | S1M10000023F08 | Staphylococcus aureus |
| 2359 | S1M10000023F10 | Staphylococcus aureus |
| 2360 | S1M10000023F11 | Staphylococcus aureus |
| 2361 | S1M10000023F12 | Staphylococcus aureus |
| 2362 | S1M10000023G02 | Staphylococcus aureus |
| 2363 | S1M10000023G03 | Staphylococcus aureus |
| 2364 | S1M10000023G06 | Staphylococcus aureus |
| 2365 | S1M10000023G07 | Staphylococcus aureus |
| 2366 | S1M10000023G08 | Staphylococcus aureus |
| 2367 | S1M10000023G09 | Staphylococcus aureus |
| 2368 | S1M10000023G11 | Staphylococcus aureus |
| 2369 | S1M10000023H02 | Staphylococcus aureus |
| 2370 | S1M10000023H06 | Staphylococcus aureus |
| 2371 | S1M10000023H07 | Staphylococcus aureus |
| 2372 | S1M10000023H09 | Staphylococcus aureus |
| 2373 | S1M10000023H10 | Staphylococcus aureus |
| 2374 | S1M10000024A02 | Staphylococcus aureus |
| 2375 | S1M10000024A04 | Staphylococcus aureus |
| 2376 | S1M10000024A07 | Staphylococcus aureus |
| 2377 | S1M10000024A08 | Staphylococcus aureus |
| 2378 | S1M10000024A11 | Staphylococcus aureus |
| 2379 | S1M10000024B05 | Staphylococcus aureus |
| 2380 | S1M10000024B06 | Staphylococcus aureus |
| 2381 | S1M10000024B08 | Staphylococcus aureus |
| 2382 | S1M10000024B09 | Staphylococcus aureus |
| 2383 | S1M10000024B10 | Staphylococcus aureus |
| 2384 | S1M10000024C02 | Staphylococcus aureus |
| 2385 | S1M10000024C04 | Staphylococcus aureus |
| 2386 | S1M10000024C07 | Staphylococcus aureus |
| 2387 | S1M10000024D02 | Staphylococcus aureus |
| 2388 | S1M10000024D03 | Staphylococcus aureus |
| 2389 | S1M10000024D10 | Staphylococcus aureus |
| 2390 | S1M10000024D11 | Staphylococcus aureus |
| 2391 | S1M10000024E03 | Staphylococcus aureus |
| 2392 | S1M10000024E05 | Staphylococcus aureus |
| 2393 | S1M10000024E06 | Staphylococcus aureus |
| 2394 | S1M10000024E07 | Staphylococcus aureus |
| 2395 | S1M10000024E08 | Staphylococcus aureus |
| 2396 | S1M10000024F02 | Staphylococcus aureus |
| 2397 | S1M10000024F03 | Staphylococcus aureus |
| 2398 | S1M10000024F05 | Staphylococcus aureus |
| 2399 | S1M10000024F08 | Staphylococcus aureus |
| 2400 | S1M10000024F10 | Staphylococcus aureus |
| 2401 | S1M10000024G05 | Staphylococcus aureus |
| 2402 | S1M10000024G06 | Staphylococcus aureus |
| 2403 | S1M10000024G07 | Staphylococcus aureus |
| 2404 | S1M10000024G08 | Staphylococcus aureus |
| 2405 | S1M10000024G10 | Staphylococcus aureus |
| 2406 | S1M10000024G12 | Staphylococcus aureus |
| 2407 | S1M10000024H02 | Staphylococcus aureus |
| 2408 | S1M10000024H04 | Staphylococcus aureus |
| 2409 | S1M10000024H07 | Staphylococcus aureus |
| 2410 | S1M10000024H08 | Staphylococcus aureus |
| 2411 | S1M10000025A03 | Staphylococcus aureus |
| 2412 | S1M10000025A08 | Staphylococcus aureus |
| 2413 | S1M10000025A09 | Staphylococcus aureus |
| 2414 | S1M10000025A10 | Staphylococcus aureus |
| 2415 | S1M10000025B01 | Staphylococcus aureus |
| 2416 | S1M10000025B02 | Staphylococcus aureus |
| 2417 | S1M10000025B03 | Staphylococcus aureus |
| 2418 | S1M10000025B05 | Staphylococcus aureus |
| 2419 | S1M10000025B06 | Staphylococcus aureus |
| 2420 | S1M10000025B09 | Staphylococcus aureus |
| 2421 | S1M10000025B12 | Staphylococcus aureus |
| 2422 | S1M10000025C01 | Staphylococcus aureus |
| 2423 | S1M10000025C03 | Staphylococcus aureus |
| 2424 | S1M10000025C05 | Staphylococcus aureus |
| 2425 | S1M10000025C09 | Staphylococcus aureus |
| 2426 | S1M10000025C10 | Staphylococcus aureus |
| 2427 | S1M10000025C11 | Staphylococcus aureus |
| 2428 | S1M10000025D01 | Staphylococcus aureus |
| 2429 | S1M10000025D03 | Staphylococcus aureus |
| 2430 | S1M10000025D04 | Staphylococcus aureus |
| 2431 | S1M10000025D06 | Staphylococcus aureus |
| 2432 | S1M10000025D08 | Staphylococcus aureus |
| 2433 | S1M10000025D09 | Staphylococcus aureus |
| 2434 | S1M10000025D10 | Staphylococcus aureus |
| 2435 | S1M10000025E01 | Staphylococcus aureus |
| 2436 | S1M10000025E04 | Staphylococcus aureus |
| 2437 | S1M10000025E09 | Staphylococcus aureus |
| 2438 | S1M10000025E11 | Staphylococcus aureus |
| 2439 | S1M10000025F03 | Staphylococcus aureus |
| 2440 | S1M10000025F05 | Staphylococcus aureus |
| 2441 | S1M10000025F08 | Staphylococcus aureus |
| 2442 | S1M10000025F09 | Staphylococcus aureus |
| 2443 | S1M1000002SF10 | Staphylococcus aureus |
| 2444 | S1M1000002SF12 | Staphylococcus aureus |
| 2445 | S1M10000025G04 | Staphylococcus aureus |
| 2446 | S1M10000025G06 | Staphylococcus aureus |
| 2447 | S1M10000025G10 | Staphylococcus aureus |
| 2448 | S1M10000025H05 | Staphylococcus aureus |
| 2449 | S1M10000025H06 | Staphylococcus aureus |
| 2450 | S1M10000025H07 | Staphylococcus aureus |
| 2451 | S1M10000025H10 | Staphylococcus aureus |
| 2452 | S1M10000026A02 | Staphylococcus aureus |
| 2453 | S1M10000026A04 | Staphylococcus aureus |
| 2454 | S1M10000026A05 | Staphylococcus aureus |
| 2455 | S1M10000026A06 | Staphylococcus aureus |
| 2456 | S1M10000026A07 | Staphylococcus aureus |
| 2457 | S1M10000026A08 | Staphylococcus aureus |
| 2458 | S1M10000026A09 | Staphylococcus aureus |
| 2459 | S1M10000026A10 | Staphylococcus aureus |
| 2460 | S1M10000026A11 | Staphylococcus aureus |
| 2461 | S1M10000026B02 | Staphylococcus aureus |
| 2462 | S1M10000026B03 | Staphylococcus aureus |
| 2463 | S1M10000026B05 | Staphylococcus aureus |
| 2464 | S1M10000026B06 | Staphylococcus aureus |
| 2465 | S1M10000026B07 | Staphylococcus aureus |
| 2466 | S1M10000026B10 | Staphylococcus aureus |
| 2467 | S1M10000026B11 | Staphylococcus aureus |
| 2468 | S1M10000026B12 | Staphylococcus aureus |
| 2469 | S1M10000026C01 | Staphylococcus aureus |
| 2470 | S1M10000026C06 | Staphylococcus aureus |
| 2471 | S1M10000026C07 | Staphylococcus aureus |
| 2472 | S1M10000026C08 | Staphylococcus aureus |
| 2473 | S1M10000026C11 | Staphylococcus aureus |
| 2474 | S1M10000026C12 | Staphylococcus aureus |
| 2475 | S1M10000026D04 | Staphylococcus aureus |
| 2476 | S1M10000026D05 | Staphylococcus aureus |
| 2477 | S1M10000026D06 | Staphylococcus aureus |
| 2478 | S1M10000026D07 | Staphylococcus aureus |
| 2479 | S1M10000026D08 | Staphylococcus aureus |
| 2480 | S1M10000026D10 | Staphylococcus aureus |
| 2481 | S1M10000026D12 | Staphylococcus aureus |
| 2482 | S1M10000026E01 | Staphylococcus aureus |
| 2483 | S1M10000026E07 | Staphylococcus aureus |
| 2484 | S1M10000026E09 | Staphylococcus aureus |
| 2485 | S1M10000026E10 | Staphylococcus aureus |
| 2486 | S1M10000026E11 | Staphylococcus aureus |
| 2487 | S1M10000026E12 | Staphylococcus aureus |
| 2488 | S1M10000026F01 | Staphylococcus aureus |
| 2489 | S1M10000026F03 | Staphylococcus aureus |
| 2490 | S1M10000026F04 | Staphylococcus aureus |
| 2491 | S1M10000026F05 | Staphylococcus aureus |
| 2492 | S1M10000026F06 | Staphylococcus aureus |
| 2493 | S1M10000026F07 | Staphylococcus aureus |
| 2494 | S1M10000026F08 | Staphylococcus aureus |
| 2495 | S1M10000026F09 | Staphylococcus aureus |
| 2496 | S1M10000026F10 | Staphylococcus aureus |
| 2497 | S1M10000026F11 | Staphylococcus aureus |
| 2498 | S1M10000026F12 | Staphylococcus aureus |
| 2499 | S1M10000026G01 | Staphylococcus aureus |
| 2500 | S1M10000026G03 | Staphylococcus aureus |
| 2501 | S1M10000026G04 | Staphylococcus aureus |
| 2502 | S1M10000026G05 | Staphylococcus aureus |
| 2503 | S1M10000026G06 | Staphylococcus aureus |
| 2504 | S1M10000026G07 | Staphylococcus aureus |
| 2505 | S1M10000026G09 | Staphylococcus aureus |
| 2506 | S1M10000026G10 | Staphylococcus aureus |
| 2507 | S1M10000026G12 | Staphylococcus aureus |
| 2508 | S1M10000026H01 | Staphylococcus aureus |
| 2509 | S1M10000026H02 | Staphylococcus aureus |
| 2510 | S1M10000026H03 | Staphylococcus aureus |
| 2511 | S1M10000026H04 | Staphylococcus aureus |
| 2512 | S1M10000026H05 | Staphylococcus aureus |
| 2513 | S1M10000026H07 | Staphylococcus aureus |
| 2514 | S1M10000026H09 | Staphylococcus aureus |
| 2515 | S1M10000026H10 | Staphylococcus aureus |
| 2516 | S1M10000027A04 | Staphylococcus aureus |
| 2517 | S1M10000027A05 | Staphylococcus aureus |
| 2518 | S1M10000027A08 | Staphylococcus aureus |
| 2519 | S1M10000027A11 | Staphylococcus aureus |
| 2520 | S1M10000027B04 | Staphylococcus aureus |
| 2521 | S1M10000027B06 | Staphylococcus aureus |
| 2522 | S1M10000027B07 | Staphylococcus aureus |
| 2523 | S1M10000027B08 | Staphylococcus aureus |
| 2524 | S1M10000027B09 | Staphylococcus aureus |
| 2525 | S1M10000027B11 | Staphylococcus aureus |
| 2526 | S1M10000027C02 | Staphylococcus aureus |
| 2527 | S1M10000027C04 | Staphylococcus aureus |
| 2528 | S1M10000027C05 | Staphylococcus aureus |
| 2529 | S1M10000027C06 | Staphylococcus aureus |
| 2530 | S1M10000027C08 | Staphylococcus aureus |
| 2531 | S1M10000027C09 | Staphylococcus aureus |
| 2532 | S1M10000027D02 | Staphylococcus aureus |
| 2533 | S1M10000027D03 | Staphylococcus aureus |
| 2534 | S1M10000027D05 | Staphylococcus aureus |
| 2535 | S1M10000027D06 | Staphylococcus aureus |
| 2536 | S1M10000027D08 | Staphylococcus aureus |
| 2537 | S1M10000027D09 | Staphylococcus aureus |
| 2538 | S1M10000027D10 | Staphylococcus aureus |
| 2539 | S1M10000027D11 | Staphylococcus aureus |
| 2540 | S1M10000027E05 | Staphylococcus aureus |
| 2541 | S1M10000027E06 | Staphylococcus aureus |
| 2542 | S1M10000027E07 | Staphylococcus aureus |
| 2543 | S1M10000027E08 | Staphylococcus aureus |
| 2544 | S1M10000027E09 | Staphylococcus aureus |
| 2545 | S1M10000027E11 | Staphylococcus aureus |
| 2546 | S1M10000027F01 | Staphylococcus aureus |
| 2547 | S1M10000027F02 | Staphylococcus aureus |
| 2548 | S1M10000027F05 | Staphylococcus aureus |
| 2549 | S1M10000027F06 | Staphylococcus aureus |
| 2550 | S1M10000027F08 | Staphylococcus aureus |
| 2551 | S1M10000027F09 | Staphylococcus aureus |
| 2552 | S1M10000027G03 | Staphylococcus aureus |
| 2553 | S1M10000027G04 | Staphylococcus aureus |
| 2554 | S1M10000027G05 | Staphylococcus aureus |
| 2555 | S1M10000027G06 | Staphylococcus aureus |
| 2556 | S1M10000027G07 | Staphylococcus aureus |
| 2557 | S1M10000027G09 | Staphylococcus aureus |
| 2558 | S1M10000027G11 | Staphylococcus aureus |
| 2559 | S1M10000027H02 | Staphylococcus aureus |
| 2560 | S1M10000027H04 | Staphylococcus aureus |
| 2561 | S1M10000027H05 | Staphylococcus aureus |
| 2562 | S1M10000027H06 | Staphylococcus aureus |
| 2563 | S1M10000027H07 | Staphylococcus aureus |
| 2564 | S1M10000027H08 | Staphylococcus aureus |
| 2565 | S1M10000027H09 | Staphylococcus aureus |
| 2566 | S1M10000027H10 | Staphylococcus aureus |
| 2567 | S1M10000027H11 | Staphylococcus aureus |
| 2568 | S1M10000028A02 | Staphylococcus aureus |
| 2569 | S1M10000028A04 | Staphylococcus aureus |
| 2570 | S1M10000028A06 | Staphylococcus aureus |
| 2571 | S1M10000028A08 | Staphylococcus aureus |
| 2572 | S1M10000028B01 | Staphylococcus aureus |
| 2573 | S1M10000028B02 | Staphylococcus aureus |
| 2574 | S1M10000028B03 | Staphylococcus aureus |
| 2575 | S1M10000028B04 | Staphylococcus aureus |
| 2576 | S1M10000028B05 | Staphylococcus aureus |
| 2577 | S1M10000028B06 | Staphylococcus aureus |
| 2578 | S1M10000028B08 | Staphylococcus aureus |
| 2579 | S1M10000028B09 | Staphylococcus aureus |
| 2580 | S1M10000028C02 | Staphylococcus aureus |
| 2581 | S1M10000028C04 | Staphylococcus aureus |
| 2582 | S1M10000028C05 | Staphylococcus aureus |
| 2583 | S1M10000028C06 | Staphylococcus aureus |
| 2584 | S1M10000028C08 | Staphylococcus aureus |
| 2585 | S1M10000028D03 | Staphylococcus aureus |
| 2586 | S1M10000028D04 | Staphylococcus aureus |
| 2587 | S1M10000028D06 | Staphylococcus aureus |
| 2588 | S1M10000028D07 | Staphylococcus aureus |
| 2589 | S1M10000028D08 | Staphylococcus aureus |
| 2590 | S1M10000028D09 | Staphylococcus aureus |
| 2591 | S1M10000028E01 | Staphylococcus aureus |
| 2592 | S1M10000028E03 | Staphylococcus aureus |
| 2593 | S1M10000028E08 | Staphylococcus aureus |
| 2594 | S1M10000028F01 | Staphylococcus aureus |
| 2595 | S1M10000028F03 | Staphylococcus aureus |
| 2596 | S1M10000028F04 | Staphylococcus aureus |
| 2597 | S1M10000028F05 | Staphylococcus aureus |
| 2598 | S1M10000028F06 | Staphylococcus aureus |
| 2599 | S1M10000028F07 | Staphylococcus aureus |
| 2600 | S1M10000028G01 | Staphylococcus aureus |
| 2601 | S1M10000028G02 | Staphylococcus aureus |
| 2602 | S1M10000028G03 | Staphylococcus aureus |
| 2603 | S1M10000028G04 | Staphylococcus aureus |
| 2604 | S1M10000028G05 | Staphylococcus aureus |
| 2605 | S1M10000028G06 | Staphylococcus aureus |
| 2606 | S1M10000028G08 | Staphylococcus aureus |
| 2607 | S1M10000028H03 | Staphylococcus aureus |
| 2608 | S1M10000028H04 | Staphylococcus aureus |
| 2609 | S1M10000028H05 | Staphylococcus aureus |
| 2610 | S1M10000029A02 | Staphylococcus aureus |
| 2611 | S1M10000029A04 | Staphylococcus aureus |
| 2612 | S1M10000029A09 | Staphylococcus aureus |
| 2613 | S1M10000029A10 | Staphylococcus aureus |
| 2614 | S1M10000029A11 | Staphylococcus aureus |
| 2615 | S1M10000029A12 | Staphylococcus aureus |
| 2616 | S1M10000029B02 | Staphylococcus aureus |
| 2617 | S1M10000029B03 | Staphylococcus aureus |
| 2618 | S1M10000029B04 | Staphylococcus aureus |
| 2619 | S1M10000029B05 | Staphylococcus aureus |
| 2620 | S1M10000029B06 | Staphylococcus aureus |
| 2621 | S1M10000029B08 | Staphylococcus aureus |
| 2622 | S1M10000029B10 | Staphylococcus aureus |
| 2623 | S1M10000029C02 | Staphylococcus aureus |
| 2624 | S1M10000029C03 | Staphylococcus aureus |
| 2625 | S1M10000029C05 | Staphylococcus aureus |
| 2626 | S1M10000029C07 | Staphylococcus aureus |
| 2627 | S1M10000029C09 | Staphylococcus aureus |
| 2628 | S1M10000029C10 | Staphylococcus aureus |
| 2629 | S1M10000029C12 | Staphylococcus aureus |
| 2630 | S1M10000029D02 | Staphylococcus aureus |
| 2631 | S1M10000029D05 | Staphylococcus aureus |
| 2632 | S1M10000029D09 | Staphylococcus aureus |
| 2633 | S1M10000029D10 | Staphylococcus aureus |
| 2634 | S1M10000029D12 | Staphylococcus aureus |
| 2635 | S1M10000029E02 | Staphylococcus aureus |
| 2636 | S1M10000029E05 | Staphylococcus aureus |
| 2637 | S1M10000029E10 | Staphylococcus aureus |
| 2638 | S1M10000029E11 | Staphylococcus aureus |
| 2639 | S1M10000029F01 | Staphylococcus aureus |
| 2640 | S1M10000029F02 | Staphylococcus aureus |
| 2641 | S1M10000029F04 | Staphylococcus aureus |
| 2642 | S1M10000029F09 | Staphylococcus aureus |
| 2643 | S1M10000029F10 | Staphylococcus aureus |
| 2644 | S1M10000029F11 | Staphylococcus aureus |
| 2645 | S1M10000029F12 | Staphylococcus aureus |
| 2646 | S1M10000029G01 | Staphylococcus aureus |
| 2647 | S1M10000029G02 | Staphylococcus aureus |
| 2648 | S1M10000029G03 | Staphylococcus aureus |
| 2649 | S1M10000029G05 | Staphylococcus aureus |
| 2650 | S1M10000029G07 | Staphylococcus aureus |
| 2651 | S1M10000029G08 | Staphylococcus aureus |
| 2652 | S1M10000029G12 | Staphylococcus aureus |
| 2653 | S1M10000029H01 | Staphylococcus aureus |
| 2654 | S1M10000029H05 | Staphylococcus aureus |
| 2655 | S1M10000029H06 | Staphylococcus aureus |
| 2656 | S1M10000029H08 | Staphylococcus aureus |
| 2657 | S1M10000029H09 | Staphylococcus aureus |
| 2658 | S1M10000029H10 | Staphylococcus aureus |
| 2659 | S1M10000030A02 | Staphylococcus aureus |
| 2660 | S1M10000030A05 | Staphylococcus aureus |
| 2661 | S1M10000030A09 | Staphylococcus aureus |
| 2662 | S1M10000030A10 | Staphylococcus aureus |
| 2663 | S1M10000030A11 | Staphylococcus aureus |
| 2664 | S1M10000030B02 | Staphylococcus aureus |
| 2665 | S1M10000030B05 | Staphylococcus aureus |
| 2666 | S1M10000030B07 | Staphylococcus aureus |
| 2667 | S1M10000030B09 | Staphylococcus aureus |
| 2668 | S1M10000030C02 | Staphylococcus aureus |
| 2669 | S1M10000030C03 | Staphylococcus aureus |
| 2670 | S1M10000030C04 | Staphylococcus aureus |
| 2671 | S1M10000030C05 | Staphylococcus aureus |
| 2672 | S1M10000030C08 | Staphylococcus aureus |
| 2673 | S1M10000030C09 | Staphylococcus aureus |
| 2674 | S1M10000030C10 | Staphylococcus aureus |
| 2675 | S1M10000030C12 | Staphylococcus aureus |
| 2676 | S1M10000030D01 | Staphylococcus aureus |
| 2677 | S1M10000030D02 | Staphylococcus aureus |
| 2678 | S1M10000030D03 | Staphylococcus aureus |
| 2679 | S1M10000030D05 | Staphylococcus aureus |
| 2680 | S1M10000030D06 | Staphylococcus aureus |
| 2681 | S1M10000030D07 | Staphylococcus aureus |
| 2682 | S1M10000030D09 | Staphylococcus aureus |
| 2683 | S1M10000030D10 | Staphylococcus aureus |
| 2684 | S1M1000003OD11 | Staphylococcus aureus |
| 2685 | S1M10000030E02 | Staphylococcus aureus |
| 2686 | S1M10000030E06 | Staphylococcus aureus |
| 2687 | S1M10000030E07 | Staphylococcus aureus |
| 2688 | S1M10000030E11 | Staphylococcus aureus |
| 2689 | S1M10000030E12 | Staphylococcus aureus |
| 2690 | S1M10000030F01 | Staphylococcus aureus |
| 2691 | S1M10000030F07 | Staphylococcus aureus |
| 2692 | S1M10000030F08 | Staphylococcus aureus |
| 2693 | S1M10000030F09 | Staphylococcus aureus |
| 2694 | S1M10000030F10 | Staphylococcus aureus |
| 2695 | S1M10000030G03 | Staphylococcus aureus |
| 2696 | S1M10000030G05 | Staphylococcus aureus |
| 2697 | S1M10000030G07 | Staphylococcus aureus |
| 2698 | S1M10000030G08 | Staphylococcus aureus |
| 2699 | S1M10000030G09 | Staphylococcus aureus |
| 2700 | S1M10000030G10 | Staphylococcus aureus |
| 2701 | S1M10000030G11 | Staphylococcus aureus |
| 2702 | S1M10000030G12 | Staphylococcus aureus |
| 2703 | S1M10000030H01 | Staphylococcus aureus |
| 2704 | S1M10000030H02 | Staphylococcus aureus |
| 2705 | S1M10000030H03 | Staphylococcus aureus |
| 2706 | S1M10000030H05 | Staphylococcus aureus |
| 2707 | S1M10000030H07 | Staphylococcus aureus |
| 2708 | S1M10000030H09 | Staphylococcus aureus |
| 2709 | S1M10000031A03 | Staphylococcus aureus |
| 2710 | S1M10000031A08 | Staphylococcus aureus |
| 2711 | S1M10000031A10 | Staphylococcus aureus |
| 2712 | S1M10000031B01 | Staphylococcus aureus |
| 2713 | S1M10000031B02 | Staphylococcus aureus |
| 2714 | S1M10000031B04 | Staphylococcus aureus |
| 2715 | S1M10000031B11 | Staphylococcus aureus |
| 2716 | S1M10000031B12 | Staphylococcus aureus |
| 2717 | S1M10000031C04 | Staphylococcus aureus |
| 2718 | S1M10000031C07 | Staphylococcus aureus |
| 2719 | S1M10000031C09 | Staphylococcus aureus |
| 2720 | S1M10000031C11 | Staphylococcus aureus |
| 2721 | S1M10000031D06 | Staphylococcus aureus |
| 2722 | S1M10000031D07 | Staphylococcus aureus |
| 2723 | S1M10000031DO8 | Staphylococcus aureus |
| 2724 | S1M10000031D09 | Staphylococcus aureus |
| 2725 | S1M10000031E02 | Staphylococcus aureus |
| 2726 | S1M10000031E03 | Staphylococcus aureus |
| 2727 | S1M10000031EO4 | Staphylococcus aureus |
| 2728 | S1M10000031E07 | Staphylococcus aureus |
| 2729 | S1M10000031E08 | Staphylococcus aureus |
| 2730 | S1M10000031E10 | Staphylococcus aureus |
| 2731 | S1M10000031E12 | Staphylococcus aureus |
| 2732 | S1M10000031F02 | Staphylococcus aureus |
| 2733 | S1M10000031F03 | Staphylococcus aureus |
| 2734 | S1M10000031F04 | Staphylococcus aureus |
| 2735 | S1M10000031F05 | Staphylococcus aureus |
| 2736 | S1M10000031F08 | Staphylococcus aureus |
| 2737 | S1M10000031F10 | Staphylococcus aureus |
| 2738 | S1M10000031F11 | Staphylococcus aureus |
| 2739 | S1M10000031F12 | Staphylococcus aureus |
| 2740 | S1M10000031G02 | Staphylococcus aureus |
| 2741 | S1M10000031G03 | Staphylococcus aureus |
| 2742 | S1M10000031G04 | Staphylococcus aureus |
| 2743 | S1M10000031G06 | Staphylococcus aureus |
| 2744 | S1M10000031G09 | Staphylococcus aureus |
| 2745 | S1M10000031G10 | Staphylococcus aureus |
| 2746 | S1M10000031G11 | Staphylococcus aureus |
| 2747 | S1M10000031H01 | Staphylococcus aureus |
| 2748 | S1M10000031H02 | Staphylococcus aureus |
| 2749 | S1M10000031H06 | Staphylococcus aureus |
| 2750 | S1M10000031H09 | Staphylococcus aureus |
| 2751 | S1M10000031H11 | Staphylococcus aureus |
| 2752 | S1M10000032A03 | Staphylococcus aureus |
| 2753 | S1M10000032A05 | Staphylococcus aureus |
| 2754 | S1M10000032A06 | Staphylococcus aureus |
| 2755 | S1M10000032A07 | Staphylococcus aureus |
| 2756 | S1M10000032A08 | Staphylococcus aureus |
| 2757 | S1M10000032A10 | Staphylococcus aureus |
| 2758 | S1M10000032B01 | Staphylococcus aureus |
| 2759 | S1M10000032B05 | Staphylococcus aureus |
| 2760 | S1M10000032B07 | Staphylococcus aureus |
| 2761 | S1M10000032B08 | Staphylococcus aureus |
| 2762 | S1M10000032B11 | Staphylococcus aureus |
| 2763 | S1M10000032B12 | Staphylococcus aureus |
| 2764 | S1M10000032C01 | Staphylococcus aureus |
| 2765 | S1M10000032C03 | Staphylococcus aureus |
| 2766 | S1M10000032C04 | Staphylococcus aureus |
| 2767 | S1M10000032C05 | Staphylococcus aureus |
| 2768 | S1M10000032C09 | Staphylococcus aureus |
| 2769 | S1M10000032C10 | Staphylococcus aureus |
| 2770 | S1M10000032C11 | Staphylococcus aureus |
| 2771 | S1M10000032C12 | Staphylococcus aureus |
| 2772 | S1M10000032D03 | Staphylococcus aureus |
| 2773 | S1M10000032D06 | Staphylococcus aureus |
| 2774 | S1M10000032D07 | Staphylococcus aureus |
| 2775 | S1M10000032D09 | Staphylococcus aureus |
| 2776 | S1M10000032D11 | Staphylococcus aureus |
| 2777 | S1M10000032E02 | Staphylococcus aureus |
| 2778 | S1M10000032E03 | Staphylococcus aureus |
| 2779 | S1M10000032E04 | Staphylococcus aureus |
| 2780 | S1M10000032E06 | Staphylococcus aureus |
| 2781 | S1M10000032E08 | Staphylococcus aureus |
| 2782 | S1M10000032E09 | Staphylococcus aureus |
| 2783 | S1M10000032E10 | Staphylococcus aureus |
| 2784 | S1M10000032E11 | Staphylococcus aureus |
| 2785 | S1M10000032E12 | Staphylococcus aureus |
| 2786 | S1M10000032F01 | Staphylococcus aureus |
| 2787 | S1M10000032F04 | Staphylococcus aureus |
| 2788 | S1M10000032F05 | Staphylococcus aureus |
| 2789 | S1M10000032F10 | Staphylococcus aureus |
| 2790 | S1M10000032F11 | Staphylococcus aureus |
| 2791 | S1M10000032F12 | Staphylococcus aureus |
| 2792 | S1M10000032G02 | Staphylococcus aureus |
| 2793 | S1M10000032G03 | Staphylococcus aureus |
| 2794 | S1M10000032G04 | Staphylococcus aureus |
| 2795 | S1M10000032G06 | Staphylococcus aureus |
| 2796 | S1M10000032G08 | Staphylococcus aureus |
| 2797 | S1M10000032G10 | Staphylococcus aureus |
| 2798 | S1M10000032G12 | Staphylococcus aureus |
| 2799 | S1M10000032H01 | Staphylococcus aureus |
| 2800 | S1M10000032H04 | Staphylococcus aureus |
| 2801 | S1M10000032H07 | Staphylococcus aureus |
| 2802 | S1M10000032H09 | Staphylococcus aureus |
| 2803 | S1M10000032H11 | Staphylococcus aureus |
| 2804 | S1M10000033A02 | Staphylococcus aureus |
| 2805 | S1M10000033A07 | Staphylococcus aureus |
| 2806 | S1M10000033A08 | Staphylococcus aureus |
| 2807 | S1M10000033A10 | Staphylococcus aureus |
| 2808 | S1M10000033B02 | Staphylococcus aureus |
| 2809 | S1M10000033B07 | Staphylococcus aureus |
| 2810 | S1M10000033B08 | Staphylococcus aureus |
| 2811 | S1M10000033B11 | Staphylococcus aureus |
| 2812 | S1M10000033B12 | Staphylococcus aureus |
| 2813 | S1M10000033C04 | Staphylococcus aureus |
| 2814 | S1M10000033D02 | Staphylococcus aureus |
| 2815 | S1M10000033D03 | Staphylococcus aureus |
| 2816 | S1M10000033D04 | Staphylococcus aureus |
| 2817 | S1M10000033D05 | Staphylococcus aureus |
| 2818 | S1M10000033D06 | Staphylococcus aureus |
| 2819 | S1M10000033D10 | Staphylococcus aureus |
| 2820 | S1M10000033D12 | Staphylococcus aureus |
| 2821 | S1M10000033E04 | Staphylococcus aureus |
| 2822 | S1M10000033E10 | Staphylococcus aureus |
| 2823 | S1M10000033E12 | Staphylococcus aureus |
| 2824 | S1M10000033F02 | Staphylococcus aureus |
| 2825 | S1M10000033F03 | Staphylococcus aureus |
| 2826 | S1M10000033F06 | Staphylococcus aureus |
| 2827 | S1M10000033F07 | Staphylococcus aureus |
| 2828 | S1M10000033F09 | Staphylococcus aureus |
| 2829 | S1M10000033F11 | Staphylococcus aureus |
| 2830 | S1M10000033G05 | Staphylococcus aureus |
| 2831 | S1M10000033G07 | Staphylococcus aureus |
| 2832 | S1M10000033G09 | Staphylococcus aureus |
| 2833 | S1M10000033G10 | Staphylococcus aureus |
| 2834 | S1M10000033G11 | Staphylococcus aureus |
| 2835 | S1M10000033G12 | Staphylococcus aureus |
| 2836 | S1M10000033H01 | Staphylococcus aureus |
| 2837 | S1M10000033H02 | Staphylococcus aureus |
| 2838 | S1M10000033H03 | Staphylococcus aureus |
| 2839 | S1M10000033H07 | Staphylococcus aureus |
| 2840 | S1M10000033H08 | Staphylococcus aureus |
| 2841 | S1M10000033H09 | Staphylococcus aureus |
| 2842 | S1M10000033H10 | Staphylococcus aureus |
| 2843 | S1M10000033H11 | Staphylococcus aureus |
| 2844 | S1M10000034A02 | Staphylococcus aureus |
| 2845 | S1M10000034A03 | Staphylococcus aureus |
| 2846 | S1M10000034A04 | Staphylococcus aureus |
| 2847 | S1M10000034A05 | Staphylococcus aureus |
| 2848 | S1M10000034A08 | Staphylococcus aureus |
| 2849 | S1M10000034A09 | Staphylococcus aureus |
| 2850 | S1M10000034A11 | Staphylococcus aureus |
| 2851 | S1M10000034A12 | Staphylococcus aureus |
| 2852 | S1M10000034B03 | Staphylococcus aureus |
| 2853 | S1M10000034B05 | Staphylococcus aureus |
| 2854 | S1M10000034B06 | Staphylococcus aureus |
| 2855 | S1M10000034B07 | Staphylococcus aureus |
| 2856 | S1M10000034B08 | Staphylococcus aureus |
| 2857 | S1M10000034B09 | Staphylococcus aureus |
| 2858 | S1M10000034B10 | Staphylococcus aureus |
| 2859 | S1M10000034B12 | Staphylococcus aureus |
| 2860 | S1M10000034C02 | Staphylococcus aureus |
| 2861 | S1M10000034C06 | Staphylococcus aureus |
| 2862 | S1M10000034C07 | Staphylococcus aureus |
| 2863 | S1M10000034C09 | Staphylococcus aureus |
| 2864 | S1M10000034C12 | Staphylococcus aureus |
| 2865 | S1M10000034D01 | Staphylococcus aureus |
| 2866 | S1M10000034D05 | Staphylococcus aureus |
| 2867 | S1M10000034D06 | Staphylococcus aureus |
| 2868 | S1M10000034D07 | Staphylococcus aureus |
| 2869 | S1M10000034D08 | Staphylococcus aureus |
| 2870 | S1M10000034D10 | Staphylococcus aureus |
| 2871 | S1M10000034D11 | Staphylococcus aureus |
| 2872 | S1M10000034D12 | Staphylococcus aureus |
| 2873 | S1M10000034E01 | Staphylococcus aureus |
| 2874 | S1M10000034E02 | Staphylococcus aureus |
| 2875 | S1M10000034E04 | Staphylococcus aureus |
| 2876 | S1M10000034E05 | Staphylococcus aureus |
| 2877 | S1M10000034E06 | Staphylococcus aureus |
| 2878 | S1M10000034E07 | Staphylococcus aureus |
| 2879 | S1M10000034E10 | Staphylococcus aureus |
| 2880 | S1M10000034E11 | Staphylococcus aureus |
| 2881 | S1M10000034E12 | Staphylococcus aureus |
| 2882 | S1M10000034F01 | Staphylococcus aureus |
| 2883 | S1M10000034F02 | Staphylococcus aureus |
| 2884 | S1M10000034F03 | Staphylococcus aureus |
| 2885 | S1M10000034F04 | Staphylococcus aureus |
| 2886 | S1M10000034F05 | Staphylococcus aureus |
| 2887 | S1M10000034F07 | Staphylococcus aureus |
| 2888 | S1M10000034F08 | Staphylococcus aureus |
| 2889 | S1M10000034F09 | Staphylococcus aureus |
| 2890 | S1M10000034F10 | Staphylococcus aureus |
| 2891 | S1M10000034F12 | Staphylococcus aureus |
| 2892 | S1M10000034G02 | Staphylococcus aureus |
| 2893 | S1M10000034G03 | Staphylococcus aureus |
| 2894 | S1M10000034G06 | Staphylococcus aureus |
| 2895 | S1M10000034G07 | Staphylococcus aureus |
| 2896 | S1M10000034G08 | Staphylococcus aureus |
| 2897 | S1M10000034G09 | Staphylococcus aureus |
| 2898 | S1M10000034G11 | Staphylococcus aureus |
| 2899 | S1M10000034G12 | Staphylococcus aureus |
| 2900 | S1M10000034H01 | Staphylococcus aureus |
| 2901 | S1M10000034H02 | Staphylococcus aureus |
| 2902 | S1M10000034H03 | Staphylococcus aureus |
| 2903 | S1M10000034H06 | Staphylococcus aureus |
| 2904 | S1M10000034H07 | Staphylococcus aureus |
| 2905 | S1M10000034H08 | Staphylococcus aureus |
| 2906 | S1M10000034H09 | Staphylococcus aureus |
| 2907 | S1M10000034H10 | Staphylococcus aureus |
| 2908 | S1M10000035A03 | Staphylococcus aureus |
| 2909 | S1M10000035A08 | Staphylococcus aureus |
| 2910 | S1M10000035A09 | Staphylococcus aureus |
| 2911 | S1M10000035A10 | Staphylococcus aureus |
| 2912 | S1M10000035A11 | Staphylococcus aureus |
| 2913 | S1M10000035A12 | Staphylococcus aureus |
| 2914 | S1M10000035B01 | Staphylococcus aureus |
| 2915 | S1M10000035B03 | Staphylococcus aureus |
| 2916 | S1M10000035B04 | Staphylococcus aureus |
| 2917 | S1M10000035B08 | Staphylococcus aureus |
| 2918 | S1M10000035B11 | Staphylococcus aureus |
| 2919 | S1M10000035C01 | Staphylococcus aureus |
| 2920 | S1M10000035C02 | Staphylococcus aureus |
| 2921 | S1M10000035C04 | Staphylococcus aureus |
| 2922 | S1M10000035C06 | Staphylococcus aureus |
| 2923 | S1M10000035C11 | Staphylococcus aureus |
| 2924 | S1M10000035D01 | Staphylococcus aureus |
| 2925 | S1M10000035D04 | Staphylococcus aureus |
| 2926 | S1M10000035D06 | Staphylococcus aureus |
| 2927 | S1M10000035D09 | Staphylococcus aureus |
| 2928 | S1M10000035D12 | Staphylococcus aureus |
| 2929 | S1M10000035E02 | Staphylococcus aureus |
| 2930 | S1M10000035E03 | Staphylococcus aureus |
| 2931 | S1M10000035E04 | Staphylococcus aureus |
| 2932 | S1M10000035E08 | Staphylococcus aureus |
| 2933 | S1M10000035E09 | Staphylococcus aureus |
| 2934 | S1M10000035E12 | Staphylococcus aureus |
| 2935 | S1M10000035F03 | Staphylococcus aureus |
| 2936 | S1M10000035F04 | Staphylococcus aureus |
| 2937 | S1M10000035F09 | Staphylococcus aureus |
| 2938 | S1M10000035F12 | Staphylococcus aureus |
| 2939 | S1M10000035G02 | Staphylococcus aureus |
| 2940 | S1M10000035G09 | Staphylococcus aureus |
| 2941 | S1M10000035G11 | Staphylococcus aureus |
| 2942 | S1M10000035G12 | Staphylococcus aureus |
| 2943 | S1M10000035H01 | Staphylococcus aureus |
| 2944 | S1M10000035H07 | Staphylococcus aureus |
| 2945 | S1M10000035H08 | Staphylococcus aureus |
| 2946 | S1M10000035H09 | Staphylococcus aureus |
| 2947 | S1M10000035H10 | Staphylococcus aureus |
| 2948 | S1M10000035H11 | Staphylococcus aureus |
| 2949 | S1M10000036A02 | Staphylococcus aureus |
| 2950 | S1M10000036A03 | Staphylococcus aureus |
| 2951 | S1M10000036A04 | Staphylococcus aureus |
| 2952 | S1M10000036A05 | Staphylococcus aureus |
| 2953 | S1M10000036A08 | Staphylococcus aureus |
| 2954 | S1M10000036A11 | Staphylococcus aureus |
| 2955 | S1M10000036A12 | Staphylococcus aureus |
| 2956 | S1M10000036B04 | Staphylococcus aureus |
| 2957 | S1M10000036B06 | Staphylococcus aureus |
| 2958 | S1M10000036B07 | Staphylococcus aureus |
| 2959 | S1M10000036B08 | Staphylococcus aureus |
| 2960 | S1M10000036B11 | Staphylococcus aureus |
| 2961 | S1M10000036B12 | Staphylococcus aureus |
| 2962 | S1M10000036C01 | Staphylococcus aureus |
| 2963 | S1M10000036C03 | Staphylococcus aureus |
| 2964 | S1M10000036C04 | Staphylococcus aureus |
| 2965 | S1M10000036C05 | Staphylococcus aureus |
| 2966 | S1M10000036C06 | Staphylococcus aureus |
| 2967 | S1M10000036C07 | Staphylococcus aureus |
| 2968 | S1M10000036C09 | Staphylococcus aureus |
| 2969 | S1M10000036C10 | Staphylococcus aureus |
| 2970 | S1M10000036D02 | Staphylococcus aureus |
| 2971 | S1M10000036D03 | Staphylococcus aureus |
| 2972 | S1M10000036D06 | Staphylococcus aureus |
| 2973 | S1M10000036D08 | Staphylococcus aureus |
| 2974 | S1M10000036D10 | Staphylococcus aureus |
| 2975 | S1M10000036D11 | Staphylococcus aureus |
| 2976 | S1M10000036D12 | Staphylococcus aureus |
| 2977 | S1M10000036E06 | Staphylococcus aureus |
| 2978 | S1M10000036E08 | Staphylococcus aureus |
| 2979 | S1M10000036E11 | Staphylococcus aureus |
| 2980 | S1M10000036F06 | Staphylococcus aureus |
| 2981 | S1M10000036F07 | Staphylococcus aureus |
| 2982 | S1M10000036F08 | Staphylococcus aureus |
| 2983 | S1M10000036F09 | Staphylococcus aureus |
| 2984 | S1M10000036F10 | Staphylococcus aureus |
| 2985 | S1M10000036F11 | Staphylococcus aureus |
| 2986 | S1M10000036G03 | Staphylococcus aureus |
| 2987 | S1M10000036G07 | Staphylococcus aureus |
| 2988 | S1M10000036G08 | Staphylococcus aureus |
| 2989 | S1M10000036G11 | Staphylococcus aureus |
| 2990 | S1M10000036H01 | Staphylococcus aureus |
| 2991 | S1M10000036H02 | Staphylococcus aureus |
| 2992 | S1M10000036H03 | Staphylococcus aureus |
| 2993 | S1M10000036H04 | Staphylococcus aureus |
| 2994 | S1M10000036H05 | Staphylococcus aureus |
| 2995 | S1M10000036H06 | Staphylococcus aureus |
| 2996 | S1M10000036H08 | Staphylococcus aureus |
| 2997 | S1M10000036H11 | Staphylococcus aureus |
| 2998 | S1M10000037A02 | Staphylococcus aureus |
| 2999 | S1M10000037A03 | Staphylococcus aureus |
| 3000 | S1M10000037A06 | Staphylococcus aureus |
| 3001 | S1M10000037A08 | Staphylococcus aureus |
| 3002 | S1M10000037A09 | Staphylococcus aureus |
| 3003 | S1M10000037A11 | Staphylococcus aureus |
| 3004 | S1M10000037A12 | Staphylococcus aureus |
| 3005 | S1M10000037B03 | Staphylococcus aureus |
| 3006 | S1M10000037B04 | Staphylococcus aureus |
| 3007 | S1M10000037B05 | Staphylococcus aureus |
| 3008 | S1M10000037B06 | Staphylococcus aureus |
| 3009 | S1M10000037B07 | Staphylococcus aureus |
| 3010 | S1M10000037B08 | Staphylococcus aureus |
| 3011 | S1M10000037B10 | Staphylococcus aureus |
| 3012 | S1M10000037B11 | Staphylococcus aureus |
| 3013 | S1M10000037B12 | Staphylococcus aureus |
| 3014 | S1M10000037C05 | Staphylococcus aureus |
| 3015 | S1M10000037C06 | Staphylococcus aureus |
| 3016 | S1M10000037C07 | Staphylococcus aureus |
| 3017 | S1M10000037C08 | Staphylococcus aureus |
| 3018 | S1M10000037C09 | Staphylococcus aureus |
| 3019 | S1M10000037C10 | Staphylococcus aureus |
| 3020 | S1M10000037D04 | Staphylococcus aureus |
| 3021 | S1M10000037D05 | Staphylococcus aureus |
| 3022 | S1M10000037D06 | Staphylococcus aureus |
| 3023 | S1M10000037D09 | Staphylococcus aureus |
| 3024 | S1M10000037D12 | Staphylococcus aureus |
| 3025 | S1M10000037E02 | Staphylococcus aureus |
| 3026 | S1M10000037E03 | Staphylococcus aureus |
| 3027 | S1M10000037E06 | Staphylococcus aureus |
| 3028 | S1M10000037E08 | Staphylococcus aureus |
| 3029 | S1M10000037E09 | Staphylococcus aureus |
| 3030 | S1M10000037E10 | Staphylococcus aureus |
| 3031 | S1M10000037E11 | Staphylococcus aureus |
| 3032 | S1M10000037E12 | Staphylococcus aureus |
| 3033 | S1M10000037F02 | Staphylococcus aureus |
| 3034 | S1M10000037F03 | Staphylococcus aureus |
| 3035 | S1M10000037F04 | Staphylococcus aureus |
| 3036 | S1M10000037F05 | Staphylococcus aureus |
| 3037 | S1M10000037F06 | Staphylococcus aureus |
| 3038 | S1M10000037F07 | Staphylococcus aureus |
| 3039 | S1M10000037F08 | Staphylococcus aureus |
| 3040 | S1M10000037F09 | Staphylococcus aureus |
| 3041 | S1M10000037F10 | Staphylococcus aureus |
| 3042 | S1M10000037G01 | Staphylococcus aureus |
| 3043 | S1M10000037G02 | Staphylococcus aureus |
| 3044 | S1M10000037G03 | Staphylococcus aureus |
| 3045 | S1M10000037G06 | Staphylococcus aureus |
| 3046 | S1M10000037G07 | Staphylococcus aureus |
| 3047 | S1M10000037G08 | Staphylococcus aureus |
| 3048 | 51M10000037G10 | Staphylococcus aureus |
| 3049 | S1M10000037H02 | Staphylococcus aureus |
| 3050 | S1M10000037H03 | Staphylococcus aureus |
| 3051 | S1M10000037H05 | Staphylococcus aureus |
| 3052 | S1M10000037H07 | Staphylococcus aureus |
| 3053 | S1M10000037H08 | Staphylococcus aureus |
| 3054 | S1M10000037H09 | Staphylococcus aureus |
| 3055 | S1M10000037H11 | Staphylococcus aureus |
| 3056 | S1M10000038A04 | Staphylococcus aureus |
| 3057 | S1M10000038A07 | Staphylococcus aureus |
| 3058 | S1M10000038A08 | Staphylococcus aureus |
| 3059 | S1M10000038A09 | Staphylococcus aureus |
| 3060 | S1M10000038A11 | Staphylococcus aureus |
| 3061 | S1M10000038A12 | Staphylococcus aureus |
| 3062 | S1M10000038B01 | Staphylococcus aureus |
| 3063 | S1M10000038B03 | Staphylococcus aureus |
| 3064 | S1M10000038B07 | Staphylococcus aureus |
| 3065 | S1M10000038B08 | Staphylococcus aureus |
| 3066 | S1M10000038B09 | Staphylococcus aureus |
| 3067 | S1M10000038B12 | Staphylococcus aureus |
| 3068 | S1M10000038C01 | Staphylococcus aureus |
| 3069 | S1M10000038C02 | Staphylococcus aureus |
| 3070 | S1M10000038C06 | Staphylococcus aureus |
| 3071 | S1M10000038C08 | Staphylococcus aureus |
| 3072 | S1M10000038C10 | Staphylococcus aureus |
| 3073 | S1M10000038C11 | Staphylococcus aureus |
| 3074 | S1M10000038C12 | Staphylococcus aureus |
| 3075 | S1M10000038D02 | Staphylococcus aureus |
| 3076 | S1M10000038D05 | Staphylococcus aureus |
| 3077 | S1M10000038D07 | Staphylococcus aureus |
| 3078 | S1M10000038D08 | Staphylococcus aureus |
| 3079 | S1M10000038D09 | Staphylococcus aureus |
| 3080 | S1M10000038D10 | Staphylococcus aureus |
| 3081 | S1M10000038D11 | Staphylococcus aureus |
| 3082 | S1M10000038D12 | Staphylococcus aureus |
| 3083 | S1M10000038E01 | Staphylococcus aureus |
| 3084 | S1M10000038E02 | Staphylococcus aureus |
| 3085 | S1M10000038E03 | Staphylococcus aureus |
| 3086 | S1M10000038E04 | Staphylococcus aureus |
| 3087 | S1M10000038E05 | Staphylococcus aureus |
| 3088 | S1M10000038E06 | Staphylococcus aureus |
| 3089 | S1M10000038E07 | Staphylococcus aureus |
| 3090 | S1M10000038E10 | Staphylococcus aureus |
| 3091 | S1M10000038E12 | Staphylococcus aureus |
| 3092 | S1M10000038F03 | Staphylococcus aureus |
| 3093 | S1M10000038F04 | Staphylococcus aureus |
| 3094 | S1M10000038F05 | Staphylococcus aureus |
| 3095 | S1M10000038F06 | Staphylococcus aureus |
| 3096 | S1M10000038F08 | Staphylococcus aureus |
| 3097 | S1M10000038F09 | Staphylococcus aureus |
| 3098 | S1M10000038F10 | Staphylococcus aureus |
| 3099 | S1M10000038F11 | Staphylococcus aureus |
| 3100 | S1M10000038F12 | Staphylococcus aureus |
| 3101 | S1M10000038G01 | Staphylococcus aureus |
| 3102 | S1M10000038G03 | Staphylococcus aureus |
| 3103 | S1M10000038G04 | Staphylococcus aureus |
| 3104 | S1M10000038G06 | Staphylococcus aureus |
| 3105 | S1M10000038G08 | Staphylococcus aureus |
| 3106 | S1M10000038G10 | Staphylococcus aureus |
| 3107 | S1M10000038G11 | Staphylococcus aureus |
| 3108 | S1M10000038G12 | Staphylococcus aureus |
| 3109 | S1M10000038H03 | Staphylococcus aureus |
| 3110 | S1M10000038H07 | Staphylococcus aureus |
| 3111 | S1M10000038H09 | Staphylococcus aureus |
| 3112 | S1M10000038H11 | Staphylococcus aureus |
| 3113 | S1M10000039A02 | Staphylococcus aureus |
| 3114 | S1M10000039A05 | Staphylococcus aureus |
| 3115 | S1M10000039A07 | Staphylococcus aureus |
| 3116 | S1M10000039A08 | Staphylococcus aureus |
| 3117 | S1M10000039A11 | Staphylococcus aureus |
| 3118 | S1M10000039A12 | Staphylococcus aureus |
| 3119 | S1M10000039B02 | Staphylococcus aureus |
| 3120 | S1M10000039B06 | Staphylococcus aureus |
| 3121 | S1M10000039B07 | Staphylococcus aureus |
| 3122 | S1M10000039B10 | Staphylococcus aureus |
| 3123 | S1M10000039B12 | Staphylococcus aureus |
| 3124 | S1M10000039C04 | Staphylococcus aureus |
| 3125 | S1M10000039C06 | Staphylococcus aureus |
| 3126 | S1M10000039C07 | Staphylococcus aureus |
| 3127 | S1M10000039C08 | Staphylococcus aureus |
| 3128 | S1M10000039C09 | Staphylococcus aureus |
| 3129 | S1M10000039C10 | Staphylococcus aureus |
| 3130 | S1M10000039C11 | Staphylococcus aureus |
| 3131 | S1M10000039D02 | Staphylococcus aureus |
| 3132 | S1M10000039D09 | Staphylococcus aureus |
| 3133 | S1M10000039D10 | Staphylococcus aureus |
| 3134 | S1M10000039E01 | Staphylococcus aureus |
| 3135 | S1M10000039E08 | Staphylococcus aureus |
| 3136 | S1M10000039E09 | Staphylococcus aureus |
| 3137 | S1M10000039E10 | Staphylococcus aureus |
| 3138 | S1M10000039E11 | Staphylococcus aureus |
| 3139 | S1M10000039F02 | Staphylococcus aureus |
| 3140 | S1M10000039F03 | Staphylococcus aureus |
| 3141 | S1M10000039F05 | Staphylococcus aureus |
| 3142 | S1M10000039F07 | Staphylococcus aureus |
| 3143 | S1M10000039F08 | Staphylococcus aureus |
| 3144 | S1M10000039F09 | Staphylococcus aureus |
| 3145 | S1M10000039F10 | Staphylococcus aureus |
| 3146 | S1M10000039F12 | Staphylococcus aureus |
| 3147 | S1M10000039G03 | Staphylococcus aureus |
| 3148 | S1M10000039G04 | Staphylococcus aureus |
| 3149 | S1M10000039G07 | Staphylococcus aureus |
| 3150 | S1M10000039G10 | Staphylococcus aureus |
| 3151 | S1M10000039H02 | Staphylococcus aureus |
| 3152 | S1M10000039H03 | Staphylococcus aureus |
| 3153 | S1M10000039H04 | Staphylococcus aureus |
| 3154 | S1M10000039H06 | Staphylococcus aureus |
| 3155 | S1M10000039H07 | Staphylococcus aureus |
| 3156 | S1M10000039H08 | Staphylococcus aureus |
| 3157 | S1M10000040A04 | Staphylococcus aureus |
| 3158 | S1M10000040A05 | Staphylococcus aureus |
| 3159 | S1M10000040A07 | Staphylococcus aureus |
| 3160 | S1M10000040A08 | Staphylococcus aureus |
| 3161 | S1M10000040A10 | Staphylococcus aureus |
| 3162 | S1M10000040A11 | Staphylococcus aureus |
| 3163 | S1M10000040B01 | Staphylococcus aureus |
| 3164 | S1M10000040B03 | Staphylococcus aureus |
| 3165 | S1M10000040B07 | Staphylococcus aureus |
| 3166 | S1M10000040B11 | Staphylococcus aureus |
| 3167 | S1M10000040C03 | Staphylococcus aureus |
| 3168 | S1M10000040C04 | Staphylococcus aureus |
| 3169 | S1M10000040C05 | Staphylococcus aureus |
| 3170 | S1M10000040C06 | Staphylococcus aureus |
| 3171 | S1M10000040C07 | Staphylococcus aureus |
| 3172 | S1M10000040C08 | Staphylococcus aureus |
| 3173 | S1M10000040C10 | Staphylococcus aureus |
| 3174 | S1M10000040C11 | Staphylococcus aureus |
| 3175 | S1M10000040D01 | Staphylococcus aureus |
| 3176 | S1M10000040D03 | Staphylococcus aureus |
| 3177 | S1M10000040D08 | Staphylococcus aureus |
| 3178 | S1M10000040D09 | Staphylococcus aureus |
| 3179 | S1M10000040D11 | Staphylococcus aureus |
| 3180 | S1M10000040E01 | Staphylococcus aureus |
| 3181 | S1M10000040E02 | Staphylococcus aureus |
| 3182 | S1M10000040E04 | Staphylococcus aureus |
| 3183 | S1M10000040E05 | Staphylococcus aureus |
| 3184 | S1M10000040E06 | Staphylococcus aureus |
| 3185 | S1M10000040E07 | Staphylococcus aureus |
| 3186 | S1M10000040E09 | Staphylococcus aureus |
| 3187 | S1M10000040E10 | Staphylococcus aureus |
| 3188 | S1M10000040E11 | Staphylococcus aureus |
| 3189 | S1M10000040E12 | Staphylococcus aureus |
| 3190 | S1M10000040F01 | Staphylococcus aureus |
| 3191 | S1M10000040F02 | Staphylococcus aureus |
| 3192 | S1M10000040F03 | Staphylococcus aureus |
| 3193 | S1M10000040F04 | Staphylococcus aureus |
| 3194 | S1M10000040F05 | Staphylococcus aureus |
| 3195 | S1M10000040F06 | Staphylococcus aureus |
| 3196 | S1M10000040F08 | Staphylococcus aureus |
| 3197 | S1M10000040F09 | Staphylococcus aureus |
| 3198 | S1M10000040F12 | Staphylococcus aureus |
| 3199 | S1M10000040G01 | Staphylococcus aureus |
| 3200 | S1M10000040G02 | Staphylococcus aureus |
| 3201 | S1M10000040G04 | Staphylococcus aureus |
| 3202 | S1M10000040G07 | Staphylococcus aureus |
| 3203 | S1M10000040G08 | Staphylococcus aureus |
| 3204 | S1M10000040G12 | Staphylococcus aureus |
| 3205 | S1M10000040H02 | Staphylococcus aureus |
| 3206 | S1M10000040H03 | Staphylococcus aureus |
| 3207 | S1M10000040H04 | Staphylococcus aureus |
| 3208 | S1M10000040H05 | Staphylococcus aureus |
| 3209 | S1M10000040H07 | Staphylococcus aureus |
| 3210 | S1M10000040H10 | Staphylococcus aureus |
| 3211 | SIM10000041A03 | Staphylococcus aureus |
| 3212 | S1M10000041B02 | Staphylococcus aureus |
| 3213 | S1M10000041B03 | Staphylococcus aureus |
| 3214 | S1M10000041B05 | Staphylococcus aureus |
| 3215 | S1M10000041B06 | Staphylococcus aureus |
| 3216 | S1M10000041B07 | Staphylococcus aureus |
| 3217 | S1M10000041B12 | Staphylococcus aureus |
| 3218 | S1M10000041C08 | Staphylococcus aureus |
| 3219 | S1M10000041C10 | Staphylococcus aureus |
| 3220 | S1M10000041C11 | Staphylococcus aureus |
| 3221 | S1M10000041D06 | Staphylococcus aureus |
| 3222 | S1M10000041D07 | Staphylococcus aureus |
| 3223 | S1M10000041D08 | Staphylococcus aureus |
| 3224 | S1M10000041D10 | Staphylococcus aureus |
| 3225 | S1M10000041D12 | Staphylococcus aureus |
| 3226 | S1M10000041E03 | Staphylococcus aureus |
| 3227 | S1M10000041E06 | Staphylococcus aureus |
| 3228 | S1M10000041E09 | Staphylococcus aureus |
| 3229 | S1M10000041E12 | Staphylococcus aureus |
| 3230 | S1M10000041F03 | Staphylococcus aureus |
| 3231 | S1M10000041F11 | Staphylococcus aureus |
| 3232 | S1M10000041F12 | Staphylococcus aureus |
| 3233 | S1M10000041G01 | Staphylococcus aureus |
| 3234 | S1M10000041G06 | Staphylococcus aureus |
| 3235 | S1M10000041G08 | Staphylococcus aureus |
| 3236 | S1M10000041G10 | Staphylococcus aureus |
| 3237 | S1M10000041G11 | Staphylococcus aureus |
| 3238 | S1M10000041H01 | Staphylococcus aureus |
| 3239 | S1M10000041H04 | Staphylococcus aureus |
| 3240 | S1M10000041H05 | Staphylococcus aureus |
| 3241 | S1M10000041H07 | Staphylococcus aureus |
| 3242 | S1M10000041H08 | Staphylococcus aureus |
| 3243 | S1M10000041H09 | Staphylococcus aureus |
| 3244 | S1M10000042A04 | Staphylococcus aureus |
| 3245 | S1M10000042A05 | Staphylococcus aureus |
| 3246 | S1M10000042A06 | Staphylococcus aureus |
| 3247 | S1M10000042A07 | Staphylococcus aureus |
| 3248 | S1M10000042A09 | Staphylococcus aureus |
| 3249 | S1M10000042A11 | Staphylococcus aureus |
| 3250 | S1M10000042A12 | Staphylococcus aureus |
| 3251 | S1M10000042B02 | Staphylococcus aureus |
| 3252 | S1M10000042B03 | Staphylococcus aureus |
| 3253 | S1M10000042B06 | Staphylococcus aureus |
| 3254 | S1M10000042B07 | Staphylococcus aureus |
| 3255 | S1M10000042B08 | Staphylococcus aureus |
| 3256 | S1M10000042B09 | Staphylococcus aureus |
| 3257 | S1M10000042B10 | Staphylococcus aureus |
| 3258 | S1M10000042B11 | Staphylococcus aureus |
| 3259 | S1M10000042B12 | Staphylococcus aureus |
| 3260 | S1M10000042C02 | Staphylococcus aureus |
| 3261 | S1M10000042C06 | Staphylococcus aureus |
| 3262 | S1M10000042C10 | Staphylococcus aureus |
| 3263 | S1M10000042C11 | Staphylococcus aureus |
| 3264 | S1M10000042D04 | Staphylococcus aureus |
| 3265 | S1M10000042D07 | Staphylococcus aureus |
| 3266 | S1M10000042D10 | Staphylococcus aureus |
| 3267 | S1M10000042D11 | Staphylococcus aureus |
| 3268 | S1M10000042E03 | Staphylococcus aureus |
| 3269 | S1M10000042E06 | Staphylococcus aureus |
| 3270 | S1M10000042E08 | Staphylococcus aureus |
| 3271 | S1M10000042F01 | Staphylococcus aureus |
| 3272 | S1M10000042F02 | Staphylococcus aureus |
| 3273 | S1M10000042F05 | Staphylococcus aureus |
| 3274 | S1M10000042F06 | Staphylococcus aureus |
| 3275 | S1M10000042F08 | Staphylococcus aureus |
| 3276 | S1M10000042F09 | Staphylococcus aureus |
| 3277 | S1M10000042F10 | Staphylococcus aureus |
| 3278 | S1M10000042F11 | Staphylococcus aureus |
| 3279 | S1M10000042G01 | Staphylococcus aureus |
| 3280 | S1M10000042G03 | Staphylococcus aureus |
| 3281 | S1M10000042G08 | Staphylococcus aureus |
| 3282 | S1M10000042G09 | Staphylococcus aureus |
| 3283 | S1M10000042G12 | Staphylococcus aureus |
| 3284 | S1M10000042H05 | Staphylococcus aureus |
| 3285 | S1M10000042H07 | Staphylococcus aureus |
| 3286 | S1M10000042H11 | Staphylococcus aureus |
| 3287 | S1M10000043A02 | Staphylococcus aureus |
| 3288 | S1M10000043A03 | Staphylococcus aureus |
| 3289 | S1M10000043A04 | Staphylococcus aureus |
| 3290 | S1M10000043A06 | Staphylococcus aureus |
| 3291 | S1M10000043A07 | Staphylococcus aureus |
| 3292 | S1M10000043A08 | Staphylococcus aureus |
| 3293 | S1M10000043A10 | Staphylococcus aureus |
| 3294 | S1M10000043A11 | Staphylococcus aureus |
| 3295 | S1M10000043A12 | Staphylococcus aureus |
| 3296 | S1M10000043B01 | Staphylococcus aureus |
| 3297 | S1M10000043B02 | Staphylococcus aureus |
| 3298 | S1M10000043B07 | Staphylococcus aureus |
| 3299 | S1M10000043B08 | Staphylococcus aureus |
| 3300 | S1M10000043B09 | Staphylococcus aureus |
| 3301 | S1M10000043B10 | Staphylococcus aureus |
| 3302 | S1M10000043B12 | Staphylococcus aureus |
| 3303 | S1M10000043C02 | Staphylococcus aureus |
| 3304 | S1M10000043C07 | Staphylococcus aureus |
| 3305 | S1M10000043C11 | Staphylococcus aureus |
| 3306 | S1M10000043C12 | Staphylococcus aureus |
| 3307 | S1M10000043D01 | Staphylococcus aureus |
| 3308 | S1M10000043D02 | Staphylococcus aureus |
| 3309 | S1M10000043D04 | Staphylococcus aureus |
| 3310 | S1M10000043D10 | Staphylococcus aureus |
| 3311 | S1M10000043D12 | Staphylococcus aureus |
| 3312 | S1M10000043E02 | Staphylococcus aureus |
| 3313 | S1M10000043E03 | Staphylococcus aureus |
| 3314 | S1M10000043E05 | Staphylococcus aureus |
| 3315 | S1M10000043E07 | Staphylococcus aureus |
| 3316 | S1M10000043E08 | Staphylococcus aureus |
| 3317 | S1M10000043E10 | Staphylococcus aureus |
| 3318 | S1M10000043E11 | Staphylococcus aureus |
| 3319 | S1M10000043E12 | Staphylococcus aureus |
| 3320 | S1M10000043F01 | Staphylococcus aureus |
| 3321 | S1M10000043F05 | Staphylococcus aureus |
| 3322 | S1M10000043F07 | Staphylococcus aureus |
| 3323 | S1M10000043F08 | Staphylococcus aureus |
| 3324 | S1M10000043F09 | Staphylococcus aureus |
| 3325 | S1M10000043G01 | Staphylococcus aureus |
| 3326 | S1M10000043G04 | Staphylococcus aureus |
| 3327 | S1M10000043G05 | Staphylococcus aureus |
| 3328 | S1M10000043G09 | Staphylococcus aureus |
| 3329 | S1M10000043G10 | Staphylococcus aureus |
| 3330 | S1M10000043H01 | Staphylococcus aureus |
| 3331 | S1M10000043H03 | Staphylococcus aureus |
| 3332 | S1M10000043H04 | Staphylococcus aureus |
| 3333 | S1M10000043H05 | Staphylococcus aureus |
| 3334 | S1M10000043H06 | Staphylococcus aureus |
| 3335 | S1M10000043H09 | Staphylococcus aureus |
| 3336 | S1M10000043H10 | Staphylococcus aureus |
| 3337 | S1M10000043H11 | Staphylococcus aureus |
| 3338 | S1M10000044A02 | Staphylococcus aureus |
| 3339 | S1M10000044A06 | Staphylococcus aureus |
| 3340 | S1M10000044A08 | Staphylococcus aureus |
| 3341 | S1M10000044A09 | Staphylococcus aureus |
| 3342 | S1M10000044A11 | Staphylococcus aureus |
| 3343 | S1M10000044A12 | Staphylococcus aureus |
| 3344 | S1M10000044B01 | Staphylococcus aureus |
| 3345 | S1M10000044B02 | Staphylococcus aureus |
| 3346 | S1M10000044B05 | Staphylococcus aureus |
| 3347 | S1M10000044B06 | Staphylococcus aureus |
| 3348 | S1M10000044B08 | Staphylococcus aureus |
| 3349 | S1M10000044B11 | Staphylococcus aureus |
| 3350 | S1M10000044B12 | Staphylococcus aureus |
| 3351 | S1M10000044C04 | Staphylococcus aureus |
| 3352 | S1M10000044C06 | Staphylococcus aureus |
| 3353 | S1M10000044C07 | Staphylococcus aureus |
| 3354 | S1M10000044C08 | Staphylococcus aureus |
| 3355 | S1M10000044C11 | Staphylococcus aureus |
| 3356 | S1M10000044C12 | Staphylococcus aureus |
| 3357 | S1M10000044D01 | Staphylococcus aureus |
| 3358 | S1M10000044D04 | Staphylococcus aureus |
| 3359 | S1M10000044D06 | Staphylococcus aureus |
| 3360 | S1M10000044D08 | Staphylococcus aureus |
| 3361 | S1M10000044D09 | Staphylococcus aureus |
| 3362 | S1M10000044D10 | Staphylococcus aureus |
| 3363 | S1M10000044D11 | Staphylococcus aureus |
| 3364 | S1M10000044D12 | Staphylococcus aureus |
| 3365 | S1M10000044E01 | Staphylococcus aureus |
| 3366 | S1M10000044E02 | Staphylococcus aureus |
| 3367 | S1M10000044E06 | Staphylococcus aureus |
| 3368 | S1M10000044E07 | Staphylococcus aureus |
| 3369 | S1M10000044E09 | Staphylococcus aureus |
| 3370 | S1M10000044E10 | Staphylococcus aureus |
| 3371 | S1M10000044E11 | Staphylococcus aureus |
| 3372 | S1M10000044F02 | Staphylococcus aureus |
| 3373 | S1M10000044F06 | Staphylococcus aureus |
| 3374 | S1M10000044F08 | Staphylococcus aureus |
| 3375 | S1M10000044F10 | Staphylococcus aureus |
| 3376 | S1M10000044G02 | Staphylococcus aureus |
| 3377 | S1M10000044G05 | Staphylococcus aureus |
| 3378 | S1M10000044G08 | Staphylococcus aureus |
| 3379 | S1M10000044G10 | Staphylococcus aureus |
| 3380 | S1M10000044G11 | Staphylococcus aureus |
| 3381 | S1M10000044H06 | Staphylococcus aureus |
| 3382 | S1M10000044H07 | Staphylococcus aureus |
| 3383 | S1M10000044H08 | Staphylococcus aureus |
| 3384 | S1M10000044H09 | Staphylococcus aureus |
| 3385 | S1M10000044H10 | Staphylococcus aureus |
| 3386 | S1M10000044H11 | Staphylococcus aureus |
| 3387 | S1M10000045A02 | Staphylococcus aureus |
| 3388 | S1M10000045A06 | Staphylococcus aureus |
| 3389 | S1M10000045A07 | Staphylococcus aureus |
| 3390 | S1M10000045A08 | Staphylococcus aureus |
| 3391 | S1M10000045A12 | Staphylococcus aureus |
| 3392 | S1M10000045B01 | Staphylococcus aureus |
| 3393 | S1M10000045B02 | Staphylococcus aureus |
| 3394 | S1M10000045B03 | Staphylococcus aureus |
| 3395 | S1M10000045B07 | Staphylococcus aureus |
| 3396 | S1M10000045B10 | Staphylococcus aureus |
| 3397 | S1M10000045B11 | Staphylococcus aureus |
| 3398 | S1M10000045B12 | Staphylococcus aureus |
| 3399 | S1M10000045C02 | Staphylococcus aureus |
| 3400 | S1M10000045C03 | Staphylococcus aureus |
| 3401 | S1M10000045C04 | Staphylococcus aureus |
| 3402 | S1M10000045C05 | Staphylococcus aureus |
| 3403 | S1M10000045C07 | Staphylococcus aureus |
| 3404 | S1M10000045C09 | Staphylococcus aureus |
| 3405 | S1M10000045D01 | Staphylococcus aureus |
| 3406 | S1M10000045D03 | Staphylococcus aureus |
| 3407 | S1M10000045D07 | Staphylococcus aureus |
| 3408 | S1M10000045D08 | Staphylococcus aureus |
| 3409 | S1M10000045D09 | Staphylococcus aureus |
| 3410 | S1M10000045D10 | Staphylococcus aureus |
| 3411 | S1M10000045D11 | Staphylococcus aureus |
| 3412 | S1M10000045D12 | Staphylococcus aureus |
| 3413 | S1M10000045E04 | Staphylococcus aureus |
| 3414 | S1M10000045E05 | Staphylococcus aureus |
| 3415 | S1M10000045E08 | Staphylococcus aureus |
| 3416 | S1M10000045E09 | Staphylococcus aureus |
| 3417 | S1M10000045E10 | Staphylococcus aureus |
| 3418 | S1M10000045E11 | Staphylococcus aureus |
| 3419 | S1M10000045E12 | Staphylococcus aureus |
| 3420 | S1M10000045F04 | Staphylococcus aureus |
| 3421 | S1M10000045F05 | Staphylococcus aureus |
| 3422 | S1M10000045F08 | Staphylococcus aureus |
| 3423 | S1M10000045F11 | Staphylococcus aureus |
| 3424 | S1M10000045F12 | Staphylococcus aureus |
| 3425 | S1M10000045G03 | Staphylococcus aureus |
| 3426 | S1M10000045G06 | Staphylococcus aureus |
| 3427 | S1M10000045G07 | Staphylococcus aureus |
| 3428 | S1M10000045G08 | Staphylococcus aureus |
| 3429 | S1M10000045G10 | Staphylococcus aureus |
| 3430 | S1M10000045G12 | Staphylococcus aureus |
| 3431 | S1M10000045H06 | Staphylococcus aureus |
| 3432 | S1M10000045H10 | Staphylococcus aureus |
| 3433 | S1M10000045H11 | Staphylococcus aureus |
| 3434 | S1M10000046A03 | Staphylococcus aureus |
| 3435 | S1M10000046A04 | Staphylococcus aureus |
| 3436 | S1M10000046A06 | Staphylococcus aureus |
| 3437 | S1M10000046A08 | Staphylococcus aureus |
| 3438 | S1M10000046A09 | Staphylococcus aureus |
| 3439 | S1M10000046A11 | Staphylococcus aureus |
| 3440 | S1M10000046A12 | Staphylococcus aureus |
| 3441 | S1M10000046B01 | Staphylococcus aureus |
| 3442 | S1M10000046B03 | Staphylococcus aureus |
| 3443 | S1M10000046B04 | Staphylococcus aureus |
| 3444 | S1M10000046B05 | Staphylococcus aureus |
| 3445 | S1M10000046B07 | Staphylococcus aureus |
| 3446 | S1M10000046B08 | Staphylococcus aureus |
| 3447 | S1M10000046B09 | Staphylococcus aureus |
| 3448 | S1M10000046B11 | Staphylococcus aureus |
| 3449 | S1M10000046B12 | Staphylococcus aureus |
| 3450 | S1M10000046C02 | Staphylococcus aureus |
| 3451 | S1M10000046C04 | Staphylococcus aureus |
| 3452 | S1M10000046C05 | Staphylococcus aureus |
| 3453 | S1M10000046C06 | Staphylococcus aureus |
| 3454 | S1M10000046C07 | Staphylococcus aureus |
| 3455 | S1M10000046C08 | Staphylococcus aureus |
| 3456 | S1M10000046C11 | Staphylococcus aureus |
| 3457 | S1M10000046C12 | Staphylococcus aureus |
| 3458 | S1M10000046D01 | Staphylococcus aureus |
| 3459 | S1M10000046D02 | Staphylococcus aureus |
| 3460 | S1M10000046D03 | Staphylococcus aureus |
| 3461 | S1M10000046D04 | Staphylococcus aureus |
| 3462 | S1M10000046D05 | Staphylococcus aureus |
| 3463 | S1M10000046D08 | Staphylococcus aureus |
| 3464 | S1M10000046D09 | Staphylococcus aureus |
| 3465 | S1M10000046D10 | Staphylococcus aureus |
| 3466 | S1M10000046D11 | Staphylococcus aureus |
| 3467 | S1M10000046D12 | Staphylococcus aureus |
| 3468 | S1M10000046E01 | Staphylococcus aureus |
| 3469 | S1M10000046E02 | Staphylococcus aureus |
| 3470 | S1M10000046E04 | Staphylococcus aureus |
| 3471 | S1M10000046E07 | Staphylococcus aureus |
| 3472 | S1M10000046E08 | Staphylococcus aureus |
| 3473 | S1M10000046E10 | Staphylococcus aureus |
| 3474 | S1M10000046F01 | Staphylococcus aureus |
| 3475 | S1M10000046F02 | Staphylococcus aureus |
| 3476 | S1M10000046F05 | Staphylococcus aureus |
| 3477 | S1M10000046F06 | Staphylococcus aureus |
| 3478 | S1M10000046F08 | Staphylococcus aureus |
| 3479 | S1M10000046F09 | Staphylococcus aureus |
| 3480 | S1M10000046F10 | Staphylococcus aureus |
| 3481 | S1M10000046F12 | Staphylococcus aureus |
| 3482 | S1M10000046G01 | Staphylococcus aureus |
| 3483 | S1M10000046G02 | Staphylococcus aureus |
| 3484 | S1M10000046G03 | Staphylococcus aureus |
| 3485 | S1M10000046G04 | Staphylococcus aureus |
| 3486 | S1M10000046G07 | Staphylococcus aureus |
| 3487 | S1M10000046G09 | Staphylococcus aureus |
| 3488 | S1M10000046G10 | Staphylococcus aureus |
| 3489 | S1M10000046H01 | Staphylococcus aureus |
| 3490 | S1M10000046H10 | Staphylococcus aureus |
| 3491 | S1M10000047A03 | Staphylococcus aureus |
| 3492 | S1M10000047A04 | Staphylococcus aureus |
| 3493 | S1M10000047A05 | Staphylococcus aureus |
| 3494 | S1M10000047A06 | Staphylococcus aureus |
| 3495 | S1M10000047A07 | Staphylococcus aureus |
| 3496 | S1M10000047A08 | Staphylococcus aureus |
| 3497 | S1M10000047A09 | Staphylococcus aureus |
| 3498 | S1M10000047A10 | Staphylococcus aureus |
| 3499 | S1M10000047A11 | Staphylococcus aureus |
| 3500 | S1M10000047A12 | Staphylococcus aureus |
| 3501 | S1M10000047B02 | Staphylococcus aureus |
| 3502 | S1M10000047B04 | Staphylococcus aureus |
| 3503 | S1M10000047BOS | Staphylococcus aureus |
| 3504 | S1M10000047B06 | Staphylococcus aureus |
| 3505 | S1M10000047B08 | Staphylococcus aureus |
| 3506 | S1M10000047B09 | Staphylococcus aureus |
| 3507 | S1M10000047B10 | Staphylococcus aureus |
| 3508 | S1M10000047B12 | Staphylococcus aureus |
| 3509 | S1M10000047C01 | Staphylococcus aureus |
| 3510 | S1M10000047C02 | Staphylococcus aureus |
| 3511 | S1M10000047C03 | Staphylococcus aureus |
| 3512 | S1M10000047C04 | Staphylococcus aureus |
| 3513 | S1M10000047C06 | Staphylococcus aureus |
| 3514 | S1M10000047C08 | Staphylococcus aureus |
| 3515 | S1M10000047C09 | Staphylococcus aureus |
| 3516 | S1M10000047C11 | Staphylococcus aureus |
| 3517 | S1M10000047C12 | Staphylococcus aureus |
| 3518 | S1M10000047D02 | Staphylococcus aureus |
| 3519 | S1M10000047D03 | Staphylococcus aureus |
| 3520 | S1M10000047D04 | Staphylococcus aureus |
| 3521 | S1M10000047D05 | Staphylococcus aureus |
| 3522 | S1M10000047D09 | Staphylococcus aureus |
| 3523 | S1M10000047D10 | Staphylococcus aureus |
| 3524 | S1M10000047D11 | Staphylococcus aureus |
| 3525 | S1M10000047D12 | Staphylococcus aureus |
| 3526 | S1M10000047E01 | Staphylococcus aureus |
| 3527 | S1M10000047E02 | Staphylococcus aureus |
| 3528 | S1M10000047E03 | Staphylococcus aureus |
| 3529 | S1M10000047E04 | Staphylococcus aureus |
| 3530 | S1M10000047E05 | Staphylococcus aureus |
| 3531 | S1M10000047E06 | Staphylococcus aureus |
| 3532 | S1M10000047E08 | Staphylococcus aureus |
| 3533 | S1M10000047E09 | Staphylococcus aureus |
| 3534 | S1M10000047E10 | Staphylococcus aureus |
| 3535 | S1M10000047E11 | Staphylococcus aureus |
| 3536 | S1M10000047E12 | Staphylococcus aureus |
| 3537 | S1M10000047F02 | Staphylococcus aureus |
| 3538 | S1M10000047F03 | Staphylococcus aureus |
| 3539 | S1M10000047F04 | Staphylococcus aureus |
| 3540 | S1M10000047F05 | Staphylococcus aureus |
| 3541 | S1M10000047F06 | Staphylococcus aureus |
| 3542 | S1M10000047F07 | Staphylococcus aureus |
| 3543 | S1M10000047F08 | Staphylococcus aureus |
| 3544 | S1M10000047F09 | Staphylococcus aureus |
| 3545 | S1M10000047F10 | Staphylococcus aureus |
| 3546 | S1M10000047F11 | Staphylococcus aureus |
| 3547 | S1M10000047F12 | Staphylococcus aureus |
| 3548 | S1M10000047G01 | Staphylococcus aureus |
| 3549 | S1M10000047G02 | Staphylococcus aureus |
| 3550 | S1M10000047G04 | Staphylococcus aureus |
| 3551 | S1M10000047G05 | Staphylococcus aureus |
| 3552 | S1M10000047G06 | Staphylococcus aureus |
| 3553 | S1M10000047G07 | Staphylococcus aureus |
| 3554 | S1M10000047G08 | Staphylococcus aureus |
| 3555 | S1M10000047G09 | Staphylococcus aureus |
| 3556 | S1M10000047G10 | Staphylococcus aureus |
| 3557 | S1M10000047H03 | Staphylococcus aureus |
| 3558 | S1M10000047H04 | Staphylococcus aureus |
| 3559 | S1M10000047H05 | Staphylococcus aureus |
| 3560 | S1M10000047H06 | Staphylococcus aureus |
| 3561 | S1M10000047H07 | Staphylococcus aureus |
| 3562 | S1M10000047H08 | Staphylococcus aureus |
| 3563 | S1M10000047H09 | Staphylococcus aureus |
| 3564 | S1M10000047H11 | Staphylococcus aureus |
| 3565 | S1M10000048A02 | Staphylococcus aureus |
| 3566 | S1M10000048A03 | Staphylococcus aureus |
| 3567 | S1M10000048A04 | Staphylococcus aureus |
| 3568 | S1M10000048A05 | Staphylococcus aureus |
| 3569 | S1M10000048A06 | Staphylococcus aureus |
| 3570 | S1M10000048A07 | Staphylococcus aureus |
| 3571 | S1M10000048A09 | Staphylococcus aureus |
| 3572 | S1M10000048A10 | Staphylococcus aureus |
| 3573 | S1M10000048A11 | Staphylococcus aureus |
| 3574 | S1M10000048A12 | Staphylococcus aureus |
| 3575 | S1M10000048B02 | Staphylococcus aureus |
| 3576 | S1M10000048B05 | Staphylococcus aureus |
| 3577 | S1M10000048B08 | Staphylococcus aureus |
| 3578 | S1M10000048B10 | Staphylococcus aureus |
| 3579 | S1M10000048B11 | Staphylococcus aureus |
| 3580 | S1M10000048B12 | Staphylococcus aureus |
| 3581 | S1M10000048C01 | Staphylococcus aureus |
| 3582 | S1M10000048C02 | Staphylococcus aureus |
| 3583 | S1M10000048C03 | Staphylococcus aureus |
| 3584 | S1M10000048C05 | Staphylococcus aureus |
| 3585 | S1M10000048C06 | Staphylococcus aureus |
| 3586 | S1M10000048C07 | Staphylococcus aureus |
| 3587 | S1M10000048C08 | Staphylococcus aureus |
| 3588 | S1M10000048C09 | Staphylococcus aureus |
| 3589 | S1M10000048C11 | Staphylococcus aureus |
| 3590 | S1M10000048D02 | Staphylococcus aureus |
| 3591 | S1M10000048D08 | Staphylococcus aureus |
| 3592 | S1M10000048D09 | Staphylococcus aureus |
| 3593 | S1M10000048D10 | Staphylococcus aureus |
| 3594 | S1M10000048D12 | Staphylococcus aureus |
| 3595 | S1M10000048E02 | Staphylococcus aureus |
| 3596 | S1M10000048E03 | Staphylococcus aureus |
| 3597 | S1M10000048E04 | Staphylococcus aureus |
| 3598 | S1M10000048E06 | Staphylococcus aureus |
| 3599 | S1M10000048E07 | Staphylococcus aureus |
| 3600 | S1M10000048E08 | Staphylococcus aureus |
| 3601 | S1M10000048E10 | Staphylococcus aureus |
| 3602 | S1M10000048F02 | Staphylococcus aureus |
| 3603 | S1M10000048F07 | Staphylococcus aureus |
| 3604 | S1M10000048F08 | Staphylococcus aureus |
| 3605 | S1M10000048F09 | Staphylococcus aureus |
| 3606 | S1M10000048F11 | Staphylococcus aureus |
| 3607 | S1M10000048F12 | Staphylococcus aureus |
| 3608 | S1M10000048G02 | Staphylococcus aureus |
| 3609 | S1M10000048G03 | Staphylococcus aureus |
| 3610 | S1M10000048G04 | Staphylococcus aureus |
| 3611 | S1M10000048G05 | Staphylococcus aureus |
| 3612 | S1M10000048G07 | Staphylococcus aureus |
| 3613 | S1M10000048G10 | Staphylococcus aureus |
| 3614 | S1M10000048G11 | Staphylococcus aureus |
| 3615 | S1M10000048H01 | Staphylococcus aureus |
| 3616 | S1M10000048H02 | Staphylococcus aureus |
| 3617 | S1M10000048H03 | Staphylococcus aureus |
| 3618 | S1M10000048H04 | Staphylococcus aureus |
| 3619 | S1M10000048H05 | Staphylococcus aureus |
| 3620 | S1M10000048H07 | Staphylococcus aureus |
| 3621 | S1M10000048H08 | Staphylococcus aureus |
| 3622 | S1M10000048H09 | Staphylococcus aureus |
| 3623 | S1M10000048H10 | Staphylococcus aureus |
| 3624 | S1M10000048H11 | Staphylococcus aureus |
| 3625 | S1M10000009E10 | Staphylococcus aureus |
| 3626 | S1M10000001F01 | Staphylococcus aureus |
| 3627 | S1M10000006B12 | Staphylococcus aureus |
| 3628 | S1M10000003D09 | Staphylococcus aureus |
| 3629 | S1M10000001D11 | Staphylococcus aureus |
| 3630 | S1M10000003B07 | Staphylococcus aureus |
| 3631 | S1M10000002A07 | Staphylococcus aureus |
| 3632 | S1M10000003F11 | Staphylococcus aureus |
| 3633 | S1M10000047C07 | Staphylococcus aureus |
| 3634 | S1M10000013F10 | Staphylococcus aureus |
| 3635 | S1M10000014D11 | Staphylococcus aureus |
| 3636 | S1M10000015F05 | Staphylococcus aureus |
| 3637 | S1M10000048D01 | Staphylococcus aureus |
| 3638 | S1M10000011C03 | Staphylococcus aureus |
| 3639 | S1M10000012F03 | Staphylococcus aureus |
| 3640 | S1M10000002F07 | Staphylococcus aureus |
| 3641 | S1M10000048G01 | Staphylococcus aureus |
| 3642 | S1M10000009G12 | Staphylococcus aureus |
| 3643 | S1M10000012D05 | Staphylococcus aureus |
| 3644 | S1M10000014D07 | Staphylococcus aureus |
| 3645 | S1M10000047C05 | Staphylococcus aureus |
| 3646 | S1M10000018D08* | Staphylococcus aureus |
| 3647 | S1M10000047B01 | Staphylococcus aureus |
| 3648 | S1M10000047H10 | Staphylococcus aureus |
| 3649 | S1M10000001A04 | Staphylococcus aureus |
| 3650 | S1M10000016E01 | Staphylococcus aureus |
| 3651 | S1M10000017E12 | Staphylococcus aureus |
| 3652 | S1M10000019B01 | Staphylococcus aureus |
| 3653 | S1M10000048F03 | Staphylococcus aureus |
| 3654 | S1M10000034A07 | Staphylococcus aureus |
| 3655 | S1M10000023G01 | Staphylococcus aureus |
| 3656 | S1M10000021G12 | Staphylococcus aureus |
| 3657 | S1M10000024E04 | Staphylococcus aureus |
| 3658 | S1M10000028H08 | Staphylococcus aureus |
| 3659 | S1M10000022B07 | Staphylococcus aureus |
| 3660 | S1M10000003A05 | Staphylococcus aureus |
| 3661 | S1M10000003AO9 | Staphylococcus aureus |
| 3662 | S1M10000003E01 | Staphylococcus aureus |
| 3663 | S1M10000004C11 | Staphylococcus aureus |
| 3664 | S1M10000007E08 | Staphylococcus aureus |
| 3665 | S1M10000021G06 | Staphylococcus aureus |
| 3666 | S1M10000024C06 | Staphylococcus aureus |
| 3667 | S1M10000024D01 | Staphylococcus aureus |
| 3668 | S1M10000027D07 | Staphylococcus aureus |
| 3669 | S1M10000027E03 | Staphylococcus aureus |
| 3670 | S1M10000027G01 | Staphylococcus aureus |
| 3671 | S1M10000029A03 | Staphylococcus aureus |
| 3672 | S1M10000032B10 | Staphylococcus aureus |
| 3673 | S1M10000032C07 | Staphylococcus aureus |
| 3674 | S1M10000038D04 | Staphylococcus aureus |
| 3675 | S1M10000047D07 | Staphylococcus aureus |
| 3676 | S1M10000048B03 | Staphylococcus aureus |
| 3677 | S1M10000048B06 | Staphylococcus aureus |
| 3678 | S1M10000048C10 | Staphylococcus aureus |
| 3679 | S1M10000048F05 | Staphylococcus aureus |
| 3680 | S4M10000001C01 | Salmonella typhimurium |
| 3681 | S4M10000002B06 | Salmonella typhimurium |
| 3682 | S4M10000002B09 | Salmonella typhimurium |
| 3683 | S4M10000002G04 | Salmonella typhimurium |
| 3684 | S4M10000002G08 | Salmonella typhimurium |
| 3685 | S4M10000005G05 | Salmonella typhimurium |
| 3686 | S4M10000005H02 | Salmonella typhimurium |
| 3687 | S4M10000006A06 | Salmonella typhimurium |
| 3688 | S4M10000006A08 | Salmonella typhimurium |
| 3689 | S4M10000006C05 | Salmonella typhimurium |
| 3690 | S4M10000006F08 | Salmonella typhimurium |
| 3691 | S4M10000007G01 | Salmonella typhimurium |
| 3692 | S4M10000008C08 | Salmonella typhimurium |
| 3693 | S4M10000008H10 | Salmonella typhimurium |
| 3694 | S4M10000009A05 | Salmonella typhimurium |
| 3695 | S4M10000010B05 | Salmonella typhimurium |
| 3696 | S4M10000010D04 | Salmonella typhimurium |
| 3697 | S4M10000010H04 | Salmonella typhimurium |
| 3698 | S4M10000011D08 | Salmonella typhimurium |
| 3699 | S4M10000011E08 | Salmonella typhimurium |
| 3700 | S4M10000012B06 | Salmonella typhimurium |
| 3701 | S4M10000012B12 | Salmonella typhimurium |
| 3702 | S4M10000012D02 | Salmonella typhimurium |
| 3703 | S4M10000013H02 | Salmonella typhimurium |
| 3704 | S4M10000014B05 | Salmonella typhimurium |
| 3705 | S4M10000014D04 | Salmonella typhimurium |
| 3706 | S4M10000014D07 | Salmonella typhimurium |
| 3707 | S4M10000014H02 | Salmonella typhimurium |
| 3708 | S4M10000015B11 | Salmonella typhimurium |
| 3709 | S4M10000015E09 | Salmonella typhimurium |
| 3710 | S4M10000016A02 | Salmonella typhimurium |
| 3711 | S4M10000018D09 | Salmonella typhimurium |
| 3712 | S4M10000018E10 | Salmonella typhimurium |
| 3713 | S4M10000018F10 | Salmonella typhimurium |
| 3714 | S4M10000018G03 | Salmonella typhimurium |
| 3715 | S4M10000018H04 | Salmonella typhimurium |
| 3716 | S4M10000019F05 | Salmonella typhimurium |
| 3717 | S4M10000019G04 | Salmonella typhimurium |
| 3718 | S4M10000019G05 | Salmonella typhimurium |
| 3719 | S4M10000019H06 | Salmonella typhimurium |
| 3720 | S4M10000020A04 | Salmonella typhimurium |
| 3721 | S4M10000020F05 | Salmonella typhimurium |
| 3722 | S4M10000020G10 | Salmonella typhimurium |
| 3723 | S4M10000022D04 | Salmonella typhimurium |
| 3724 | S4M10000022D12 | Salmonella typhimurium |
| 3725 | S4M10000022E12 | Salmonella typhimurium |
| 3726 | S4M10000022G07 | Salmonella typhimurium |
| 3727 | S4M10000022H06 | Salmonella typhimurium |
| 3728 | S4M10000023F01 | Salmonella typhimurium |
| 3729 | S4M10000024B02 | Salmonella typhimurium |
| 3730 | S4M10000024C06 | Salmonella typhimurium |
| 3731 | S4M10000024C11 | Salmonella typhimurium |
| 3732 | S4M10000024F08 | Salmonella typhimurium |
| 3733 | S4M10000024G01 | Salmonella typhimurium |
| 3734 | S4M10000024G04 | Salmonella typhimurium |
| 3735 | S4M10000024G09 | Salmonella typhimurium |
| 3736 | S4M10000024H02 | Salmonella typhimurium |
| 3737 | S4M10000025A11 | Salmonella typhimurium |
| 3738 | S4M10000025E02 | Salmonella typhimurium |
| 3739 | S4M10000025E05 | Salmonella typhimurium |
| 3740 | S4M10000025H07 | Salmonella typhimurium |
| 3741 | S4M10000026C10 | Salmonella typhimurium |
| 3742 | S4M10000026D04 | Salmonella typhimurium |
| 3743 | S4M10000026E06 | Salmonella typhimurium |
| 3744 | S4M10000026E12 | Salmonella typhimurium |
| 3745 | S4M10000027C10 | Salmonella typhimurium |
| 3746 | S4M10000027E02 | Salmonella typhimurium |
| 3747 | S4M10000029B12 | Salmonella typhimurium |
| 3748 | S4M10000029D12 | Salmonella typhimurium |
| 3749 | S4M10000030D03 | Salmonella typhimurium |
| 3750 | S4M10000030F07 | Salmonella typhimurium |
| 3751 | S4M10000030G11 | Salmonella typhimurium |
| 3752 | S4M10000032B12 | Salmonella typhimurium |
| 3753 | S4M10000033F08 | Salmonella typhimurium |
| 3754 | S4M10000033G05 | Salmonella typhimurium |
| 3755 | S4M10000033G09 | Salmonella typhimurium |
| 3756 | S4M10000034A02 | Salmonella typhimurium |
| 3757 | S4M10000034A09 | Salmonella typhimurium |
| 3758 | S4M10000034D06 | Salmonella typhimurium |
| 3759 | S4M10000034H05 | Salmonella typhimurium |
| 3760 | S4M10000034H09 | Salmonella typhimurium |
| 3761 | S4M10000035B01 | Salmonella typhimurium |
| 3762 | S4M10000035D01 | Salmonella typhimurium |
| 3763 | S4M10000035D02 | Salmonella typhimurium |
| 3764 | S4M10000035E03 | Salmonella typhimurium |
| 3765 | S4M10000035F02 | Salmonella typhimurium |
| 3766 | S4M10000035F09 | Salmonella typhimurium |
| 3767 | S4M10000036D07 | Salmonella typhimurium |
| 3768 | S4M10000036F07 | Salmonella typhimurium |
| 3769 | S4M10000037A04 | Salmonella typhimurium |
| 3770 | S4M10000037A10 | Salmonella typhimurium |
| 3771 | S4M10000037E10 | Salmonella typhimurium |
| 3772 | S4M10000037H09 | Salmonella typhimurium |
| 3773 | S4M10000001H01 | Salmonella typhimurium |
| 3774 | S4M10000002F06 | Salmonella typhimurium |
| 3775 | S4M10000008D01 | Salmonella typhimurium |
| 3776 | S4M10000009G11 | Salmonella typhimurium |
| 3777 | S4M10000011F09 | Salmonella typhimurium |
| 3778 | S4M10000020F08 | Salmonella typhimurium |
| 3779 | S4M10000021E07 | Salmonella typhimurium |
| 3780 | S4M10000022B05 | Salmonella typhimurium |
| 3781 | S4M10000025H11 | Salmonella typhimurium |
| 3782 | S4M10000026B10 | Salmonella typhimurium |
| 3783 | S4M10000026E03 | Salmonella typhimurium |
| 3784 | S4M10000029A03 | Salmonella typhimurium |
| 3785 | S4M10000029C11 | Salmonella typhimurium |
| 3786 | S4M10000030F06 | Salmonella typhimurium |
| 3787 | S4M10000032F03 | Salmonella typhimurium |
| 3788 | S4M10000032G01 | Salmonella typhimurium |
| 3789 | S4M10000034C05 | Salmonella typhimurium |
| 3790 | S4M10000034H04 | Salmonella typhimurium |
| 3791 | S4M10000035A09 | Salmonella typhimurium |
| 3792 | S4M10000035B06 | Salmonella typhimurium |
| 3793 | S4M10000035F01 | Salmonella typhimurium |
| 3794 | S4M10000037A08 | Salmonella typhimurium |
| 3795 | S4M10000037E03 | Salmonella typhimurium |

TABLE 1B

| | | | | | full length |
|---|---|---|---|---|---|
| | | | | | ORF |
| | Clone | | Gene Seq ID | | Protein Seq |
| Clone name | Seq ID | PathoSeq Locus | (protein) | Genemarked gene | ID |
| E3M10000001A02 | 8 | EFA101409 | 4934 | EFA1c0022_orf_11p | 10524 |
| E3M10000001A06 | 9 | EFA100642 | 4884 | EFA1c0041_orf_56p | 10792 |
| E3M10000001B01 | 10 | EFA101409 | 4934 | EFA1c0022_orf_11p | 10524 |
| E3M10000001B02 | 11 | EFA100739 | 4888 | EFA1c0022_orf_23p | 10537 |
| E3M10000001B02 | 11 | EFA102549 | 5000 | EFA1c0022_orf_24p | 10538 |
| E3M10000001B02 | 11 | EFA1025S1 | 5001 | EFA1c0022_orf_25p | 10539 |
| E3M10000001B05 | 12 | EFA101165 | 4922 | EFA1c0022_orf_8p | 10559 |
| E3M10000001B06 | 13 | EFA101164 | 4921 | EFA1c0022_orf_7p | 10558 |
| E3M10000001B08 | 14 | EFA100642 | 4884 | EFA1c0041_orf_56p | 10792 |
| E3M10000001B10 | 15 | EFA101409 | 4934 | EFA1c0022_orf_11p | 10524 |
| E3M10000001C02 | 16 | EFA103038 | 5017 | EFA1c0030_orf_17p | 10613 |
| E3M10000001C09 | 17 | EFA103021 | 5015 | EFA1c0030_orf_16p | 10612 |
| E3M10000001D02 | 18 | EFA101159 | 4916 | EFA1c0022_orf_2p | 10543 |
| E3M10000001D04 | 19 | EFA100742 | 4891 | EFA1c0022_orf_20p | 10534 |
| E3M10000001D04 | 19 | EFA101417 | 4942 | EFA1c0022_orf_18p | 10531 |
| E3M10000001D04 | 19 | EFA102554 | 5002 | EFA1c0022_orf_19p | 10532 |
| E3M10000001D05 | 20 | EFA100955 | 4902 | EFA1c0022_orf_28p | 10542 |
| E3M10000001D05 | 20 | EFA100978 | 4904 | EFA1c0022_orf_27p | 10541 |
| E3M10000001D09 | 21 | EFA100210 | 4870 | EFA1c0022_orf_9p | 10560 |
| E3M10000001D09 | 21 | EFA100211 | 4871 | EFA1c0022_orf_10p | 10523 |
| B3M10000001E01 | 22 | EFA101162 | 4919 | EFA1c0022_orf_5p | 10555 |
| E3M10000001E01 | 22 | EFA101163 | 4920 | EFA1c0022_orf_6p | 10557 |
| E3M10000001E02 | 23 | EFA103038 | 5017 | EFA1c0030_orf_17p | 10613 |
| E3M10000001E03 | 24 | EFA100210 | 4870 | EFA1c0022_orf_9p | 10560 |
| E3M10000001E03 | 24 | EFA100211 | 4871 | EFA1c0022_orf_10p | 10523 |
| E3M10000001E04 | 25 | EFA100642 | 4884 | EFA1c0041_orf_56p | 10792 |
| E3M10000001E08 | 26 | EFA102502 | 4995 | EFA1c0031_orf_36p | 10627 |
| E3M10000001E09 | 27 | EFA100210 | 4870 | EFA1c0022_orf_9p | 10560 |
| E3M10000001E09 | 27 | EFA100211 | 4871 | EFA1c0022_orf_10p | 10523 |
| E3M10000001F02 | 28 | EFA102502 | 4995 | EFA1c0031_orf_36p | 10627 |
| E3M10000001F04 | 29 | EFA102541 | 4998 | EFA1c0028_orf_3p | 10602 |
| E3M10000001F06 | 30 | EFA100642 | 4884 | EFA1c0041_orf_56p | 10792 |
| E3M10000001F07 | 31 | EFA101164 | 4921 | EFA1c0022_orf_7p | 10558 |
| E3M10000001G02 | 32 | EFA101409 | 4934 | EFA1c0022_orf_11p | 10524 |
| E3M10000001G03 | 33 | EFA100210 | 4870 | EFA1c0022_orf_9p | 10560 |
| E3M10000001G03 | 33 | EFA100211 | 4871 | EFA1c0022_orf_10p | 10523 |
| E3M10000001G04 | 34 | EFA101165 | 4922 | EFA1c0022_orf_8p | 10559 |
| E3M10000001G05 | 35 | EFA101160 | 4917 | EFA1c0022_orf_3p | 10549 |
| E3M10000001H02 | 36 | EFA102541 | 4998 | EFA1c0028_orf_3p | 10602 |
| E3M10000001H03 | 37 | EFA100210 | 4870 | EFA1c0022_orf_9p | 10560 |
| E3M10000001H03 | 37 | EFA100211 | 4871 | EFA1c0022_orf_10p | 10523 |
| E3M10000001H04 | 38 | EFA100742 | 4891 | EFA1c0022_orf_20p | 10534 |
| E3M10000001H04 | 38 | EFA101417 | 4942 | EFA1c0022_orf_18p | 10531 |
| E3M10000001H04 | 38 | EFA102554 | 5002 | EFA1c0022_orf_19p | 10532 |
| E3M10000004A04 | 39 | EFA101417 | 4942 | EFA1c0022_orf_18p | 10531 |
| E3M10000004A04 | 39 | EFA102554 | 5002 | EFA1c0022_orf_19p | 10532 |
| E3M10000004C03 | 40 | EFA100478 | 4880 | EFA1c0012_orf_2p | 10486 |
| E3M10000004D01 | 41 | EFA101412 | 4937 | EFA1c0022_orf_14p | 10527 |
| E3M10000004D01 | 41 | EFA101413 | 4938 | #N/A | #N/A |
| E3M10000004D01 | 41 | EFA101414 | 4939 | EFA1c0022_orf_15p | 10528 |
| E3M10000004D02 | 42 | EFA102022 | 4974 | EFA1c0044_orf_106p | 10881 |
| E3M10000004D02 | 42 | EFA102023 | 4975 | EFA1c0044_orf_107p | 10882 |
| E3M10000004D10 | 43 | EFA101162 | 4919 | EFA1c0022_orf_5p | 10555 |
| E3M10000004D10 | 43 | EFA101163 | 4920 | EFA1c0022_orf_6p | 10557 |
| E3M10000004E11 | 44 | EFA101086 | 4910 | EFA1c0240_orf_90p | 10763 |
| E3M10000004F08 | 45 | EFA102549 | 5000 | EFA1c0022_orf_24p | 10538 |
| E3M10000004F08 | 45 | EFA102551 | 5001 | EFA1c0022_orf_25p | 10539 |
| B3M10000004F10 | 46 | EFA101086 | 4910 | EFA1c0040_orf_90p | 10763 |
| E3M10000004G01 | 47 | EFA103021 | 5015 | EFA1c0030_orf_16p | 10612 |
| E3M10000004H11 | 48 | EFA102549 | 5000 | EFA1c0022_orf_24p | 10538 |
| E3M10000004H11 | 48 | EFA102551 | 5001 | EFA1c0022_orf_25p | 10539 |
| E3M10000005A07 | 49 | EFA102541 | 4998 | EFA1c0028_orf_3p | 10602 |
| E3M10000005B01 | 50 | EFA101414 | 4939 | EFA1c0022_orf_15p | 10528 |
| E3M10000005B01 | 50 | EFA101415 | 4940 | EFA1c0022_orf_16p | 10529 |
| E3M10000005B08 | 51 | EFA102549 | 5000 | EFA1c0022_orf_24p | 10538 |
| E3M10000005B08 | 51 | EFA102551 | 5001 | EFA1c0022_orf_25p | 10539 |
| E3M10000005C01 | 52 | EFA103021 | 5015 | EFA1c0030_orf_16p | 10612 |
| E3M10000005C03 | 53 | EFA102541 | 4998 | EFA1c0028_orf_3p | 10602 |
| E3M10000005C04 | 54 | EFA102186 | 4981 | EFA1c0045_orf_94p | 10949 |
| E3M10000005C04 | 54 | EFA102453 | 4993 | EFA1c0045_orf_203p | 10931 |
| E3M10000005C04 | 54 | EFA102728 | 5006 | EFA1c0045_orf_93p | 10948 |
| E3M10000005D03 | 55 | EFA102541 | 4998 | EFA1c0028_orf_3p | 10602 |
| E3M10000005D04 | 56 | EFA103021 | 5015 | EFA1c0030_orf_16p | 10612 |
| E3M10000005D10 | 57 | EFA102549 | 5000 | EFA1c0022_orf_24p | 10538 |
| E3M10000005D10 | 57 | EFA102551 | 5001 | EFA1c0022_orf_25p | 10539 |
| E3M10000005E01 | 58 | EFA102549 | 5000 | EFA1c0022_orf_24p | 10538 |
| E3M10000005E01 | 58 | EFA102551 | 5001 | EFA1c0022_orf_25p | 10539 |
| E3M10000005E02 | 59 | EFA102549 | 5000 | EFA1c0022_orf_24p | 10538 |
| E3M10000005E02 | 59 | EFA102551 | 5001 | EFA1c0022_orf_25p | 10539 |
| E3M10000005E03 | 60 | EFA102541 | 4998 | EFA1c0028_orf_3p | 10602 |
| E3M10000005E08 | 61 | EFA101403 | 4932 | EFA1c0033_orf_54p | 10662 |
| E3M10000005F07 | 62 | EFA103021 | 5015 | EFA1c0030_orf_16p | 10612 |
| E3M10000005F10 | 63 | EFA102549 | 5000 | EFA1c0022_orf_24p | 10538 |
| E3M10000005F10 | 63 | EFA102551 | 5001 | EFA1c0022_orf_25p | 10539 |
| E3M10000005G05 | 64 | EFA102549 | 5000 | EFA1c0022_orf_24p | 10538 |
| E3M10000005G05 | 64 | EFA102551 | 5001 | EFA1c0022_orf_25p | 10539 |
| E3M10000005H04 | 65 | EFA103021 | 5015 | EFA1c0030_orf_16p | 10612 |
| E3M10000006B03 | 66 | EFA101162 | 4919 | EFA1c0022_orf_5p | 10555 |
| E3M10000006B03 | 66 | EFA101163 | 4920 | EFA1c0022_orf_6p | 10557 |
| E3M10000006C01 | 67 | EFA101416 | 4941 | EFA1c0022_orf_17p | 10530 |
| E3M10000006C01 | 67 | EFA101417 | 4942 | EFA1c0022_orf_18p | 10531 |
| E3M10000006C12 | 68 | EFA102549 | 5000 | EFA1c0022_orf_24p | 10538 |
| E3M10000006C12 | 68 | EFA102551 | 5001 | EFA1c0022_orf_25p | 10539 |
| E3M10000006D03 | 69 | EFA101416 | 4941 | EFA1c0022_orf_17p | 10530 |
| E3M10000006D03 | 69 | EFA101417 | 4942 | EFA1c0022_orf_18p | 10531 |
| E3M10000006E11 | 70 | EFA102541 | 4998 | EFA1c0028_orf_3p | 10602 |
| E3M10000006E11 | 70 | EFA102542 | 4999 | EFA1c0028_orf_4p | 10603 |
| E3M10000006F04 | 71 | EFA102541 | 4998 | EFA1c0028_orf_3p | 10602 |
| E3M10000006F04 | 71 | EFA102542 | 4999 | EFA1c0028_orf_4p | 10603 |
| E3M10000006G04 | 72 | EFA101162 | 4919 | EFA1c0022_orf_5p | 10555 |
| E3M10000006G04 | 72 | EFA101163 | 4920 | EFA1c0022_orf_6p | 10557 |
| E3M10000006G12 | 73 | EFA101162 | 4919 | EFA1c0022_orf_5p | 10555 |
| E3M10000006G12 | 73 | EFA101163 | 4920 | EFA1c0022_orf_6p | 10557 |
| E3M10000006H09 | 74 | EFA100642 | 4884 | EFA1c0041_orf_56p | 10792 |
| E3M10000007A02 | 75 | EFA101162 | 4919 | EFA1c0022_orf_5p | 10555 |
| E3M10000007A02 | 75 | EFA101163 | 4920 | EFA1c0022_orf_6p | 10557 |
| E3M10000007B02 | 76 | EFA101162 | 4919 | EFA1c0022_orf_5p | 10555 |
| E3M10000007B02 | 76 | EFA101163 | 4920 | EFA1c0022_orf_6p | 10557 |
| E3M10000007B03 | 77 | EFA101162 | 4919 | EFA1c0022_orf_5p | 10555 |
| E3M10000007B03 | 77 | EFA101163 | 4920 | EFA1c0022_orf_6p | 10557 |
| E3M10000007C03 | 78 | EFA101416 | 4941 | EFA1c0022_orf_17p | 10530 |
| E3M10000007C03 | 78 | EFA101417 | 4942 | EFA1c0022_orf_18p | 10531 |
| E3M10000007C04 | 79 | EFA100642 | 4884 | EFA1c0041_orf_56p | 10792 |
| E3M10000007D03 | 80 | EFA101162 | 4919 | EFA1c0022_orf_5p | 10555 |
| E3M10000007D03 | 80 | EFA101163 | 4920 | EFA1c0022_orf_6p | 10557 |
| E3M10000007E05 | 81 | EFA100742 | 4891 | EFA1c0022_orf_20p | 10534 |
| E3M10000007E05 | 81 | EFA101417 | 4942 | EFA1c0022_orf_18p | 10531 |
| E3M10000007E05 | 81 | EFA102554 | 5002 | EFA1c0022_orf_19p | 10532 |
| E3M10000007F01 | 82 | EFA101162 | 4919 | EFA1c0022_orf_5p | 10555 |
| E3M10000007F01 | 82 | EFA101163 | 4920 | EFA1c0022_orf_6p | 10557 |
| E3M10000007F06 | 83 | EFA101162 | 4919 | EFA1c0022_orf_5p | 10555 |
| E3M10000007F06 | 83 | EFA101163 | 4920 | EFA1c0022_orf_6p | 10557 |
| E3M10000007G01 | 84 | EFA101162 | 4919 | EFA1c0022_orf_5p | 10555 |
| E3M10000007G01 | 84 | EFA101163 | 4920 | EFA1c0022_orf_6p | 10557 |
| E3M10000008C03 | 85 | EFA102501 | 4994 | EFA1c0031_orf_35p | 10626 |
| E3M10000008C08 | 86 | EFA101536 | 4946 | EFA1c0042_orf_46p | 10823 |
| E3M10000008C09 | 87 | EFA101410 | 4935 | EFA1c0022_orf_12p | 10525 |
| E3M10000008D08 | 88 | EFA102501 | 4994 | EFA1c0031_orf_35p | 10626 |
| E3M10000008E02 | 89 | EFA100783 | 4895 | EFA1c0042_orf_141p | 10811 |
| E3M10000008G05 | 90 | EFA101162 | 4919 | EFA1c0022_orf_5p | 10555 |
| E3M10000008G05 | 90 | EFA101163 | 4920 | EFA1c0022_orf_6p | 10557 |
| E3M10000008G09 | 91 | EFA103021 | 5015 | EFA1c0030_orf_16p | 10612 |
| E3M10000008G09 | 91 | EFA103038 | 5017 | EFA1c0030_orf_17p | 10613 |
| E3M10000008H02 | 92 | EFA101695 | 4954 | EFA1c0031_orf_6p | 10629 |
| E3M10000009C07 | 93 | EFA103508 | 5029 | EFA1c0033_orf_95p | 10672 |
| E3M10000009C09 | 94 | EFA100870 | 4899 | EFA1c0031_orf_36p | 10627 |
| E3M10000009D01 | 95 | EFA101410 | 4935 | EFA1c0022_orf_12p | 10525 |
| E3M10000009E02 | 96 | EFA101410 | 4935 | EFA1c0022_orf_12p | 10525 |
| E3M10000009E02 | 96 | EFA101411 | 4936 | EFA1c0022_orf_13p | 10526 |
| E3M10000009E03 | 97 | EFA101160 | 4917 | EFA1c0022_orf_3p | 10549 |
| E3M10000009E05 | 98 | EFA102501 | 4994 | EFA1c0031_orf_35p | 10626 |
| E3M10000009G02 | 99 | EFA102501 | 4994 | EFA1c0031_orf_35p | 10626 |
| E3M10000010C08 | 100 | EFA100870 | 4899 | EFA1c0031_orf_36p | 10627 |
| E3M10000010D05 | 101 | EFA100757 | 4894 | EFA1c0044_orf_27p | 10897 |
| E3M10000010F01 | 102 | EFA102551 | 5001 | EFA1c0022_orf_25p | 10539 |
| E3M10000010G05 | 103 | EFA101164 | 4921 | EFA1c0022_orf_7p | 10558 |
| E3M10000010G07 | 104 | EFA101165 | 4922 | EFA1c0022_orf_8p | 10559 |
| E3M10000010G09 | 105 | EFA103571 | 5030 | EFA1c0044_orf_101p | 10879 |
| E3M10000010G10 | 106 | EFA102091 | 4977 | EFA1c0010_orf_3p | 10481 |
| E3M1000200210H | 107 | EFA100194 | 4868 | EFA1c0022_orf_26p | 10540 |
| E3M10000011A09 | 108 | EFA103038 | 5017 | EFA1c0030_orf_17p | 10613 |
| E3M10000011B03 | 109 | EFA102091 | 4977 | EFA1c0010_orf_3p | 10481 |
| E3M10000011B09 | 110 | EFA102501 | 4994 | EFA1c0031_orf_35p | 10626 |
| E3M10000011C07 | 111 | EFA101790 | 4959 | EFA1c0042_orf_111p | 10803 |
| E3M10000011D03 | 112 | EFA100210 | 4870 | EFA1c0022_orf_9p | 10560 |
| E3M10000011D03 | 112 | EFA100211 | 4871 | EFA1c0022_orf_10p | 10523 |
| E3M10000011H02 | 113 | EFA102541 | 4998 | EFA1c0028_orf_3p | 10602 |
| E3M10000011H05 | 114 | EFA101164 | 4921 | EFA1c0022_orf_7p | 10558 |
| E3M10000012B01 | 115 | EFA100642 | 4884 | EFA1c0041_orf_56p | 10792 |
| E3M10000012B02 | 116 | EFA100151 | 4864 | EFA1c0021_orf_14p | 10516 |
| E3M10000012B07 | 117 | EFA101410 | 4935 | EFA1c0022_orf_12p | 10525 |
| E3M10000012B07 | 117 | EFA101411 | 4936 | EFA1c0022_orf_13p | 10526 |
| E3M10000012B07 | 117 | EFA101412 | 4937 | EFA1c0022_orf_14p | 10527 |
| E3M10000012B08 | 118 | EFA101409 | 4934 | EFA1c0022_orf_11p | 10524 |
| E3M10000012C01 | 119 | EFA100642 | 4884 | EFA1c0041_orf_56p | 10792 |
| E3M10000012D10 | 120 | EFA102501 | 4994 | EFA1c0031_orf_35p | 10626 |
| E3M10000012E08 | 121 | EFA101164 | 4921 | EFA1c0022_orf_7p | 10558 |
| E3M10000012F05 | 122 | EFA101162 | 4919 | EFA1c0022_orf_5p | 10555 |
| E3M10000012F06 | 123 | EFA101409 | 4934 | EFA1c0022_orf_11p | 10524 |
| E3M10000012F07 | 124 | EFA101417 | 4942 | EFA1c0022_orf_18p | 10531 |
| E3M10000012F07 | 124 | EFA102554 | 5002 | EFA1c0022_orf_19p | 10532 |
| E3M10000012F10 | 125 | EFA101410 | 4935 | EFA1c0022_orf_12p | 10525 |
| E3M10000012F10 | 125 | EFA101411 | 4936 | EFA1c0022_orf_13p | 10526 |
| E3M10000012G02 | 126 | EFA101165 | 4922 | EFA1c0022_orf_8p | 10559 |
| E3M10000012G07 | 127 | EFA101410 | 4935 | EFA1c0022_orf_12p | 10525 |
| E3M10000012G07 | 127 | EFA101411 | 4936 | EFA1c0022_orf_13p | 10526 |
| E3M10000013A06 | 128 | EFA101159 | 4916 | EFA1c0022_orf_2p | 10543 |
| E3M10000013A07 | 129 | EFA101160 | 4917 | EFA1c0022_orf_3p | 10549 |
| E3M10000013C05 | 130 | EFA101160 | 4917 | EFA1c0022_orf_3p | 10549 |
| E3M10000013C05 | 130 | EFA101161 | 4918 | EFA1c0022_orf_4p | 10551 |
| E3M10000013D02 | 131 | EFA101160 | 4917 | EFA1c0022_orf_3p | 10549 |
| E3M10000013D08 | 132 | EFA101415 | 4940 | EFA1c0022_orf_16p | 10529 |
| E3M10000013D10 | 133 | EFA100210 | 4870 | EFA1c0022_orf_9p | 10560 |
| E3M10000013D10 | 133 | EFA100211 | 4871 | EFA1c0022_orf_10p | 10523 |
| E3M10000013E02 | 134 | EFA100642 | 4884 | EFA1c0041_orf_56p | 10792 |
| E3M10000013E08 | 135 | EFA102501 | 4994 | EFA1c0031_orf_35p | 10626 |
| E3M10000013F05 | 136 | EFA102541 | 4998 | EFA1c0028_orf_3p | 10602 |
| E3M10000013F12 | 137 | EFA101164 | 4921 | EFA1c0022_orf_7p | 10558 |
| E3M10000013F12 | 137 | EFA101165 | 4922 | EFA1c0022_orf_8p | 10559 |
| E3M10000013G10 | 138 | EFA103062 | 5019 | EFA1c0030_orf_19p | 10615 |
| E3M10000013H03 | 139 | EFA101412 | 4937 | EFA1c0022_orf_14p | 10527 |
| E3M10000013H05 | 140 | EFA101163 | 4920 | EFA1c0022_orf_6p | 10557 |
| E3M10000013H10 | 141 | EFA101164 | 4921 | EFA1c0022_orf_7p | 10558 |
| E3M10000014B12 | 142 | EFA100739 | 4888 | EFA1c0022_orf_23p | 10537 |
| E3M10000014B12 | 142 | EFA102549 | 5000 | EFA1c0022_orf_24p | 10538 |
| E3M10000014B12 | 142 | EFA102551 | 5001 | EFA1c0022_orf_25p | 10539 |
| E3M10000014E12 | 143 | EFA101409 | 4934 | EFA1c0022_orf_11p | 10524 |
| E3M10000014E12 | 143 | EFA101410 | 4935 | EFA1c0022_orf_12p | 10525 |
| E3M10000014G09 | 144 | EFA100991 | 4905 | EFA1c0035_orf_60p | 10681 |
| E3M10000014G09 | 144 | EFA103033 | 5016 | EFA1c0035_orf_60p | 10681 |
| E3M10000015B04 | 145 | EFA100065 | 4863 | EFA1c0042_orf_14p | 10813 |
| E3M10000015B12 | 146 | EFA101162 | 4919 | EFA1c0022_orf_5p | 10555 |
| E3M10000015E12 | 147 | EFA100210 | 4870 | EFA1c0022_orf_9p | 10560 |
| E3M10000015E12 | 147 | EFA100211 | 4871 | EFA1c0022_orf_10p | 10523 |
| E3M10000016A03 | 148 | EFA101753 | 4957 | EFA1c0022_orf_50p | 10552 |
| E3M10000016A04 | 149 | EFA101409 | 4934 | EFA1c0022_orf_11p | 10524 |
| E3M10000016C11 | 150 | EFA101163 | 4920 | EFA1c0022_orf_6p | 10557 |
| E3M10000016C11 | 150 | EFA101164 | 4921 | EFA1c0022_orf_7p | 10558 |
| E3M10000016D03 | 151 | EFA102774 | 5009 | EFA1c0044_orf_25p | 10896 |
| E3M10000016F06 | 152 | EFA102205 | 4983 | EFA1c0041_orf_115p | 10769 |
| E3M10000016F10 | 153 | EFA101410 | 4935 | EFA1c0022_orf_12p | 10525 |
| E3M10000016F10 | 153 | EFA101411 | 4936 | EFA1c0022_orf_13p | 10526 |
| E3M10000016H05 | 154 | EFA101160 | 4917 | EFA1c0022_orf_3p | 10549 |
| E3M10000016H10 | 155 | EFA101409 | 4934 | EFA1c0022_orf_11p | 10524 |
| E3M10000017A09 | 156 | EFA101161 | 4918 | EFA1c0022_orf_4p | 10551 |
| E3M10000017A09 | 156 | EFA101162 | 4919 | EFA1c0022_orf_5p | 10555 |
| E3M10000017D09 | 157 | EFA101412 | 4937 | EFA1c0022_orf_14p | 10527 |
| E3M10000018A07 | 158 | EFA102091 | 4977 | EFA1c0010_orf_3p | 10481 |
| E3M10000018C02 | 159 | EFA100642 | 4884 | EFA1c0041_orf_56p | 10792 |
| E3M10000018E01 | 160 | EFA103021 | 5015 | EFA1c0030_orf_16p | 10612 |
| E3M10000018G09 | 161 | EFA101583 | 4949 | EFA1c0026_orf_23p | 10593 |
| E3M10000018H06 | 162 | EFA101417 | 4942 | EFA1c0022_orf_18p | 10531 |
| E3M10000019B06 | 163 | EFA100151 | 4864 | EFA1c0021_orf_14p | 10516 |
| E3M10000019D02 | 164 | EFA102022 | 4974 | EFA1c0044_orf_106p | 10881 |
| E3M10000019E03 | 165 | EFA100870 | 4899 | EFA1c0031_orf_36p | 10627 |
| E3M10000019E03 | 165 | EFA102502 | 4995 | EFA1c0031_orf_36p | 10627 |
| E3M10000019E04 | 166 | EFA102501 | 4994 | EFA1c0031_orf_35p | 10626 |
| E3M10000020G04 | 167 | EFA100870 | 4899 | EFA1c0031_orf_36p | 10627 |
| E3M10000020G04 | 167 | EFA102502 | 4995 | EFA1c0031_orf_36p | 10627 |
| E3M10000020H05 | 168 | EFA103038 | 5017 | EFA1c0030_orf_17p | 10613 |
| E3M10000021A08 | 169 | EFA101162 | 4919 | EFA1c0022_orf_5p | 10555 |
| E3M10000021A08 | 169 | EFA101163 | 4920 | EFA1c0022_orf_6p | 10557 |
| E3M10000021A11 | 170 | EFA101417 | 4942 | EFA1c0022_orf_18p | 10531 |
| E3M10000021B10 | 171 | EFA101163 | 4920 | EFA1c0022_orf_6p | 10557 |
| E3M10000021C03 | 172 | EFA102501 | 4994 | EFA1c0031_orf_35p | 10626 |
| E3M10000021C04 | 173 | EFA101161 | 4918 | EFA1c0022_orf_4p | 10551 |
| E3M10000021C08 | 174 | EFA101160 | 4917 | EFA1c0022_orf_3p | 10549 |
| E3M10000021D04 | 175 | EFA100870 | 4899 | EFA1c0031_orf_36p | 10627 |
| E3M10000021D04 | 175 | EFA102502 | 4995 | EFA1c0031_orf_36p | 10627 |
| E3M10000021E10 | 176 | EFA100704 | 4887 | EFA1c0010_orf_4p | 10482 |
| E3M10000021G04 | 177 | EFA100955 | 4902 | EFA1c0022_orf_28p | 10542 |
| E3M10000021G10 | 178 | EFA100642 | 4884 | EFA1c0041_orf_56p | 10792 |
| E3M10000021G11 | 179 | EFA101163 | 4920 | EFA1c0022_orf_6p | 10557 |
| E3M10000021H11 | 180 | EFA101163 | 4920 | EFA1c0022_orf_6p | 10557 |
| E3M10000022A04 | 181 | EFA101410 | 4935 | EFA1c0022_orf_12p | 10525 |
| E3M10000022A11 | 182 | EFA101417 | 4942 | EFA1c0022_orf_18p | 10531 |
| E3M10000022B04 | 183 | EFA101410 | 4935 | EFA1c0022_orf_12p | 10525 |
| E3M10000022B05 | 184 | EFA101410 | 4935 | EFA1c0022_orf_12p | 10525 |
| E3M10000022B05 | 184 | EFA101411 | 4936 | EFA1c0022_orf_13p | 10526 |
| E3M10000022B07 | 185 | EFA103571 | 5030 | EFA1c0044_orf_101p | 10879 |
| E3M10000022C05 | 186 | EFA101160 | 4917 | EFA1c0022_orf_3p | 10549 |
| E3M10000022C05 | 186 | EFA101161 | 4918 | EFA1c0022_orf_4p | 10551 |
| E3M10000022C06 | 187 | EFA100978 | 4904 | EFA1c0022_orf_27p | 10541 |
| E3M10000022C09 | 188 | EFA101162 | 4919 | EFA1c0022_orf_5p | 10555 |
| E3M10000022D04 | 189 | EFA101412 | 4937 | EFA1c0022_orf_14p | 10527 |
| E3M10000022F05 | 190 | EFA102501 | 4994 | EFA1c0031_orf_35p | 10626 |
| E3M10000022F06 | 191 | EFA101161 | 4918 | EFA1c0022_orf_4p | 10551 |
| E3M10000022F06 | 191 | EFA101162 | 4919 | EFA1c0022_orf_5p | 10555 |
| E3M10000022F08 | 192 | EFA101410 | 4935 | EFA1c0022_orf_12p | 10525 |
| E3M10000022G02 | 193 | EFA101022 | 4906 | EFA1c0043_orf_69p | 10875 |
| E3M10000022G12 | 194 | EFA100704 | 4887 | EFA1c0010_orf_4p | 10482 |
| E3M10000023A03 | 195 | EFA101413 | 4938 | #N/A | #N/A |
| E3M10000023A06 | 196 | EFA100978 | 4904 | EFA1c0022_orf_27p | 10541 |
| E3M10000023A07 | 197 | EFA102502 | 4995 | EFA1c0031_orf_36p | 10627 |
| E3M10000023A09 | 198 | EFA100704 | 4887 | EFA1c0010_orf_4p | 10482 |
| E3M10000023B02 | 199 | EFA101159 | 4916 | EFA1c0022_orf_2p | 10543 |
| E3M10000023B02 | 199 | EFA101160 | 4917 | EFA1c0022_orf_3p | 10549 |
| E3M10000023B06 | 200 | EFA102541 | 4998 | EFA1c0028_orf_3p | 10602 |
| E3M10000023C03 | 201 | EFA101409 | 4934 | EFA1c0022_orf_11p | 10524 |
| E3M10000023C03 | 201 | EFA101410 | 4935 | EFA1c0022_orf_12p | 10525 |
| E3M10000023C04 | 202 | EFA102541 | 4998 | EFA1c0028_orf_3p | 10602 |
| E3M10000023C06 | 203 | EFA101413 | 4938 | #N/A | #N/A |
| E3M10000023C08 | 204 | EFA100955 | 4902 | EFA1c0022_orf_28p | 10542 |
| E3M10000023C09 | 205 | EFA101159 | 4916 | EFA1c0022_orf_2p | 10543 |
| E3M10000023C09 | 205 | EFA101160 | 4917 | EFA1c0022_orf_3p | 10549 |
| E3M10000023D02 | 206 | EFA102501 | 4994 | EFA1c0031_orf_35p | 10626 |
| E3M10000023D04 | 207 | EFA101160 | 4917 | EFA1c0022_orf_3p | 10549 |
| E3M10000023D10 | 208 | EFA101413 | 4938 | #N/A | #N/A |
| E3M10000023E04 | 209 | EFA101412 | 4937 | EFA1c0022_orf_14p | 10527 |
| E3M10000023E07 | 210 | EFA101162 | 4919 | EFA1c0022_orf_5p | 10555 |
| E3M10000023E09 | 211 | EFA102501 | 4994 | EFA1c0031_orf_35p | 10626 |
| E3M10000023F02 | 212 | EFA101412 | 4937 | EFA1c0022_orf_14p | 10527 |
| E3M10000023F10 | 213 | EFA102551 | 5001 | EFA1c0022_orf_25p | 10539 |
| E3M10000023G02 | 214 | EFA101160 | 4917 | EFA1c0022_orf_3p | 10549 |
| E3M10000023G04 | 215 | EFA101414 | 4939 | EFA1c0022_orf_15p | 10528 |
| E3M10000023G10 | 216 | EFA101411 | 4936 | EFA1c0022_orf_13p | 10526 |
| E3M10000023H08 | 217 | EFA102502 | 4995 | EFA1c0031_orf_36p | 10627 |
| E3M10000024A03 | 218 | EFA101161 | 4918 | EFA1c0022_orf_4p | 10551 |
| E3M10000024A04 | 219 | EFA102006 | 4973 | EFA1c0025_orf_17p | 10580 |
| E3M10000024A08 | 220 | EFA101160 | 4917 | EFA1c0022_orf_3p | 10549 |
| E3M10000024A08 | 220 | EFA101161 | 4918 | EFA1c0022_orf_4p | 10551 |
| E3M10000024C06 | 221 | EFA102501 | 4994 | EFA1c0031_orf_35p | 10626 |
| E3M10000025A06 | 222 | EFA101160 | 4917 | EFA1c0022_orf_3p | 10549 |
| E3M10000025B01 | 223 | EFA100194 | 4868 | EFA1c0022_orf_26p | 10540 |
| E3M10000025B01 | 223 | EFA100978 | 4904 | EFA1c0022_orf_27p | 10541 |
| E3M10000025B03 | 224 | EFA101411 | 4936 | EFA1c0022_orf_13p | 10526 |
| E3M10000025B03 | 224 | EFA101412 | 4937 | EFA1c0022_orf_14p | 10527 |
| E3M10000025B05 | 225 | EFA100978 | 4904 | EFA1c0022_orf_27p | 10541 |
| E3M10000025B10 | 226 | EFA101162 | 4919 | EFA1c0022_orf_5p | 10555 |
| E3M10000025C01 | 227 | EFA102502 | 4995 | EFA1c0031_orf_36p | 10627 |
| E3M10000025C04 | 228 | EFA101159 | 4916 | EFA1c0022_orf_2p | 10543 |
| E3M10000025C05 | 229 | EFA102549 | 5000 | EFA1c0022_orf_24p | 10538 |
| E3M10000025C05 | 229 | EFA102551 | 5001 | EFA1c0022_orf_25p | 10539 |
| E3M10000025C07 | 230 | EFA100642 | 4884 | EFA1c0041_orf_56p | 10792 |
| E3M10000025C08 | 231 | EFA100870 | 4899 | EFA1c0031_orf_36p | 10627 |
| E3M10000025C08 | 231 | EFA102502 | 4995 | EFA1c0031_orf_36p | 10627 |
| E3M10000025C09 | 232 | EFA102501 | 4994 | EFA1c0031_orf_35p | 10626 |
| E3M10000025C11 | 233 | EFA101162 | 4919 | EFA1c0022_orf_5p | 10555 |
| E3M10000025D01 | 234 | EFA101160 | 4917 | EFA1c0022_orf_3p | 10549 |
| E3M10000025D01 | 234 | EFA101161 | 4918 | EFA1c0022_orf_4p | 10551 |
| E3M10000025D10 | 235 | EFA102501 | 4994 | EFA1c0031_orf_35p | 10626 |
| E3M10000025E07 | 236 | EFA101165 | 4922 | EFA1c0022_orf_8p | 10559 |
| E3M10000025E08 | 237 | EFA100955 | 4902 | EFA1c0022_orf_28p | 10542 |
| E3M10000025E12 | 238 | EFA102728 | 5006 | EFA1c0045_orf_93p | 10948 |
| E3M10000025F04 | 239 | EFA101160 | 4917 | EFA1c0022_orf_3p | 10549 |
| E3M10000025F04 | 239 | EFA101161 | 4918 | EFA1c0022_orf_4p | 10551 |
| E3M10000025F06 | 240 | EFA101410 | 4935 | EFA1c0022_orf_12p | 10525 |
| E3M10000025F06 | 240 | EFA101411 | 4936 | EFA1c0022_orf_13p | 10526 |
| E3M10000025F06 | 240 | EFA101412 | 4937 | EFA1c0022_orf_14p | 10527 |
| E3M10000025F08 | 241 | EFA103038 | 5017 | EFA1c0030_orf_17p | 10613 |
| E3M10000025F09 | 242 | EFA100704 | 4887 | EFA1c0010_orf_4p | 10482 |
| E3M10000025F10 | 243 | EFA101161 | 4918 | EFA1c0022_orf_4p | 10551 |
| E3M10000025F11 | 244 | EFA100955 | 4902 | EFA1c0022_orf_28p | 10542 |
| E3M10000025F12 | 245 | EFA101163 | 4920 | EFA1c0022_orf_6p | 10557 |
| E3M10000025G02 | 246 | EFA101164 | 4921 | EFA1c0022_orf_7p | 10558 |
| E3M10000025007 | 247 | EFA101159 | 4916 | EFA1c0022_orf_2p | 10543 |
| E3M10000025G09 | 248 | EFA102185 | 4980 | EFA1c0045_orf_95p | 10950 |
| E3M10000027A02 | 249 | EFA101416 | 4941 | EFA1c0022_orf_17p | 10530 |
| E3M10000027A07 | 250 | EFA101160 | 4917 | EFA1c0022_orf_3p | 10549 |
| E3M10000027A09 | 251 | EFA101413 | 4938 | #N/A | #N/A |
| E3M10000027A09 | 251 | EFA101414 | 4939 | EFA1c0022_orf_15p | 10528 |
| E3M10000027B07 | 252 | EFA101163 | 4920 | EFA1c0022_orf_6p | 10557 |
| E3M10000027B08 | 253 | EFA101160 | 4917 | EFA1c0022_orf_3p | 10549 |
| E3M10000027B09 | 254 | EFA100870 | 4899 | EFA1c0031_orf_36p | 10627 |
| E3M10000027B09 | 254 | EFA102502 | 4995 | EFA1c0031_orf_36p | 10627 |
| E3M10000027C02 | 255 | EFA103062 | 5019 | EFA1c0030_orf_19p | 10615 |
| E3M10000027C03 | 256 | EFA101160 | 4917 | EFA1c0022_orf_3p | 10549 |
| E3M10000027C08 | 257 | EFA101165 | 4922 | EFA1c0022_orf_8p | 10559 |
| E3M10000027D03 | 258 | EFA100870 | 4899 | EFA1c0031_orf_36p | 10627 |
| E3M10000027D03 | 258 | EFA102502 | 4995 | EFA1c0031_orf_36p | 10627 |
| E3M10000027D05 | 259 | EFA101162 | 4919 | EFA1c0022_orf_5p | 10555 |
| E3M10000027D08 | 260 | EFA103504 | 5028 | EFA1c0033_orf_94p | 10671 |
| E3M10000027D10 | 261 | EFA100704 | 4887 | EFA1c0010_orf_4p | 10482 |
| E3M10000027G01 | 262 | EFA102186 | 4981 | EFA1c0045_orf_94p | 10949 |
| E3M10000027G08 | 263 | EFA101409 | 4934 | EFA1c0022_orf_11p | 10524 |
| E3M10000027H04 | 264 | EFA101160 | 4917 | EFA1c0022_orf_3p | 10549 |
| E3M10000027H07 | 265 | EFA101161 | 4918 | EFA1c0022_orf_4p | 10551 |
| E3M10000027H07 | 265 | EFA101162 | 4919 | EFA1c0022_orf_5p | 10555 |
| E3M10000028A02 | 266 | EFA102554 | 5002 | EFA1c0022_orf_19p | 10532 |
| E3M10000028A03 | 267 | EFA102551 | 5001 | EFA1c0022_orf_25p | 10539 |
| E3M10000028A04 | 268 | EFA101410 | 4935 | EFA1c0022_orf_12p | 10525 |
| E3M10000028A04 | 268 | EFA101411 | 4936 | EFA1c0022_orf_13p | 10526 |
| E3M10000028A05 | 269 | EFA101080 | 4909 | #N/A | #N/A |
| E3M10000028A05 | 269 | EFA102915 | 5014 | EFA1c0032_orf_27p | 10640 |
| E3M10000028A06 | 270 | EFA103210 | 5022 | EFA1c0036_orf_119p | 10688 |
| E3M10000028A08 | 271 | EFA101424 | 4943 | EFA1c0041_orf_39p | 10784 |
| E3M10000028A08 | 271 | EFA101425 | 4944 | EFA1c0041_orf_40p | 10785 |
| E3M10000028B01 | 272 | EFA103021 | 5015 | EFA1c0030_orf_16p | 10612 |
| E3M10000028B02 | 273 | EFA102541 | 4998 | EFA1c0028_orf_3p | 10602 |
| E3M10000028B02 | 273 | EFA102542 | 4999 | EFA1c0028_orf_4p | 10603 |
| E3M10000028B03 | 274 | EFA101160 | 4917 | EFA1c0022_orf_3p | 10549 |
| E3M10000028B04 | 275 | EFA101161 | 4918 | EFA1c0022_orf_4p | 10551 |
| E3M10000028B05 | 276 | EFA101424 | 4943 | EFA1c0041_orf_39p | 10784 |
| E3M10000028B05 | 276 | EFA101425 | 4944 | EFA1c0041_orf_40p | 10785 |
| E3M10000028B06 | 277 | EFA103038 | 5017 | EFA1c0030_orf_17p | 10613 |
| E3M10000028B07 | 278 | EFA101160 | 4917 | EFA1c0022_orf_3p | 10549 |
| E3M10000028B08 | 279 | EFA101417 | 4942 | EFA1c0022_orf_18p | 10531 |
| E3M10000028C01 | 280 | EFA102541 | 4998 | EFA1c0028_orf_3p | 10602 |
| E3M10000028C01 | 280 | EFA102542 | 4999 | EFA1c0028_orf_4p | 10603 |
| E3M10000028C02 | 281 | EFA102541 | 4998 | EFA1c0028_orf_3p | 10602 |
| E3M10000028C02 | 281 | EFA102542 | 4999 | EFA1c0028_orf_4p | 10603 |
| E3M10000028C04 | 282 | EFA101322 | 4927 | EFA1c0030_orf_57p | 10620 |
| E3M10000028C05 | 283 | EFA101160 | 4917 | EFA1c0022_orf_3p | 10549 |
| E3M10000028C06 | 284 | EFA100151 | 4864 | EFA1c0021_orf_14p | 10516 |
| E3M10000028C07 | 285 | EFA101022 | 4906 | EFA1c0043_orf_69p | 10875 |
| E3M10000028C08 | 286 | EFA102541 | 4998 | EFA1c0028_orf_3p | 10602 |
| E3M10000028C08 | 286 | EFA102542 | 4999 | EFA1c0028_orf_4p | 10603 |
| E3M10000028D01 | 287 | EFA100194 | 4868 | EFA1c0022_orf_26p | 10540 |
| E3M10000028D01 | 287 | EFA100978 | 4904 | EFA1c0022_orf_27p | 10541 |
| E3M10000028D02 | 288 | EFA101022 | 4906 | EFA1c0043_orf_69p | 10875 |
| E3M10000028D05 | 289 | EFA101080 | 4909 | #N/A | #N/A |
| E3M10000028D06 | 290 | EFA103021 | 5015 | EFA1c0030_orf_16p | 10612 |
| E3M10000028D08 | 291 | EFA103268 | 5023 | EFA1c0010_orf_1p | 10479 |
| E3M10000028E01 | 292 | EFA101121 | 4912 | EFA1c0036_orf_112p | 10686 |
| E3M10000028E04 | 293 | EFA101370 | 4931 | EFA1c0040_orf_103p | 10738 |
| E3M10000028E07 | 294 | EFA102541 | 4998 | EFA1c0028_orf_3p | 10602 |
| E3M10000028F02 | 295 | EFA101161 | 4918 | EFA1c0022_orf_4p | 10551 |
| E3M10000028F03 | 296 | EFA100742 | 4891 | EFA1c0022_orf_20p | 10534 |
| E3M10000028F03 | 296 | EFA101417 | 4942 | EFA1c0022_orf_18p | 10531 |
| E3M10000028F03 | 296 | EFA102554 | 5002 | EFA1c0022_orf_19p | 10532 |
| E3M10000028F04 | 297 | EFA101163 | 4920 | EFA1c0022_orf_6p | 10557 |
| E3M10000028F04 | 297 | EFA101164 | 4921 | EFA1c0022_orf_7p | 10558 |
| E3M10000028F05 | 298 | EFA100704 | 4887 | EFA1c0010_orf_4p | 10482 |
| E3M10000028F06 | 299 | EFA101164 | 4921 | EFA1c0022_orf_7p | 10558 |
| E3M10000028F07 | 300 | EFA100642 | 4884 | EFA1c0041_orf_56p | 10792 |
| E3M10000028G05 | 301 | EFA100704 | 4887 | EFA1c0010_orf_4p | 10482 |
| E3M10000028G06 | 302 | EFA100748 | 4892 | EFA1c0011_orf_10p | 10483 |
| E3M10000028G07 | 303 | EFA101410 | 4935 | EFA1c0022_orf_12p | 10525 |
| E3M10000028G07 | 303 | EFA101411 | 4936 | EFA1c0022_orf_13p | 10526 |
| E3M10000028H04 | 304 | EFA101409 | 4934 | EFA1c0022_orf_11p | 10524 |
| E3M10000028H07 | 305 | EFA103062 | 5019 | EFA1c0030_orf_19p | 10615 |
| E3M10000029A02 | 306 | EFA101160 | 4917 | EFA1c0022_orf_3p | 10549 |
| E3M10000029A04 | 307 | EFA100210 | 4870 | EFA1c0022_orf_9p | 10560 |
| E3M10000029A05 | 308 | EFA100642 | 4884 | EFA1c0041_orf_56p | 10792 |
| E3M10000029A10 | 309 | EFA102091 | 4977 | EFA1c0010_orf_3p | 10481 |
| E3M10000029A11 | 310 | EFA101413 | 4938 | #N/A | #N/A |
| E3M10000029B01 | 311 | EFA103295 | 5024 | EFA1c0032_orf_1p | 10633 |
| E3M10000029B02 | 312 | EFA101160 | 4917 | EFA1c0022_orf_3p | 10549 |
| E3M10000029B05 | 313 | EFA103038 | 5017 | EFA1c0030_orf_17p | 10613 |
| E3M10000029B06 | 314 | EFA100914 | 4900 | EFA1c0024_orf_9p | 10579 |
| E3M10000029B08 | 315 | EFA102338 | 4987 | EFA1c0032_orf_8p | 10651 |
| E3M10000029B11 | 316 | EFA100397 | 4877 | EFA1c0041_orf_148p | 10773 |
| E3M10000029B12 | 317 | EFA100642 | 4884 | EFA1c0041_orf_56p | 10792 |
| E3M10000029C01 | 318 | EFA102541 | 4998 | EFA1c0028_orf_3p | 10602 |
| E3M10000029C02 | 319 | EFA102788 | 5011 | EFA1c0033_orf_41p | 10661 |
| E3M10000029C03 | 320 | EFA102253 | 4984 | EFA1c0038_orf_85p | 10727 |
| E3M10000029C04 | 321 | EFA102503 | 4996 | EFA1c0032_orf_32p | 10643 |
| E3M10000029C05 | 322 | EFA100399 | 4878 | EFA1c0041_orf_104p | 10766 |
| E3M10000029C06 | 323 | EFA101414 | 4939 | EFA1c0022_orf_15p | 10528 |
| E3M10000029C06 | 323 | EFA101415 | 4940 | EFA1c0022_orf_16p | 10529 |
| E3M10000029C07 | 324 | EFA102352 | 4990 | EFA1c0032_orf_21p | 10635 |
| E3M10000029C07 | 324 | EFA102353 | 4991 | EFA1c0032_orf_22p | 10636 |
| E3M10000029C08 | 325 | EFA101868 | 4966 | EFA1c0042_orf_69p | 10829 |
| E3M10000029C09 | 326 | EFA101121 | 4912 | EFA1c0036_orf_112p | 10686 |
| E3M10000029C10 | 327 | EFA102656 | 5004 | EFA1c0039_orf_26p | 10734 |
| E3M10000029C12 | 328 | EFA101121 | 4912 | EFA1c0036_orf_112p | 10686 |
| E3M10000029D01 | 329 | EFA101080 | 4909 | #N/A | #N/A |
| E3M10000029D03 | 330 | EFA101160 | 4917 | EFA1c0022_orf_3p | 10549 |
| E3M10000029D04 | 331 | EFA102656 | 5004 | EFA1c0039_orf_26p | 10734 |
| E3M10000029D05 | 332 | EFA100210 | 4870 | EFA1c0022_orf_9p | 10560 |
| E3M10000029D06 | 333 | EFA100210 | 4870 | EFA1c0022_orf_9p | 10560 |
| E3M10000029D06 | 333 | EFA101165 | 4922 | EFA1c0022_orf_8p | 10559 |
| E3M10000029D08 | 334 | EFA102736 | 5007 | EFA1c0022_orf_60p | 10556 |
| E3M10000029D12 | 335 | EFA101410 | 4935 | EFA1c0022_orf_12p | 10525 |
| E3M10000029E01 | 336 | EFA101404 | 4933 | EFA1c0033_orf_55p | 10663 |
| E3M10000029E02 | 337 | EFA102051 | 4976 | #N/A | #N/A |
| E3M10000029E03 | 338 | EFA102502 | 4995 | EFA1c0031_orf_36p | 10627 |
| E3M10000029E05 | 339 | EFA101686 | 4953 | EFA1c0045_orf_63p | 10940 |
| E3M10000029E07 | 340 | EFA100919 | 4901 | EFA1c0013_orf_12p | 10491 |
| E3M10000029E08 | 341 | EFA101022 | 4906 | EFA1c0043_orf_69p | 10875 |
| E3M10000029E09 | 342 | EFA102656 | 5004 | EFA1c0039_orf_26p | 10734 |
| E3M10000029E12 | 343 | EFA100397 | 4877 | EFA1c0041_orf_148p | 10773 |
| E3M10000029F01 | 344 | EFA100023 | 4862 | EFA1c0017_orf_1p | 10505 |
| E3M10000029F05 | 345 | EFA102503 | 4996 | EFA1c0032_orf_32p | 10643 |
| E3M10000029F06 | 346 | EFA101795 | 4962 | EFA1c0045_orf_165p | 10922 |
| E3M10000029F09 | 347 | EFA100689 | 4886 | EFA1c0038_orf_54p | 10717 |
| E3M10000029F10 | 348 | EFA100919 | 4901 | EFA1c0013_orf_12p | 10491 |
| E3M10000029F11 | 349 | EFA102541 | 4998 | EFA1c0028_orf_3p | 10602 |
| E3M10000029F12 | 350 | EFA102282 | 4985 | EFA1c0038_orf_89p | 10729 |
| E3M10000029G01 | 351 | EFA100394 | 4876 | EFA1c0034_orf_6p | 10675 |
| E3M10000029G04 | 352 | EFA102656 | 5004 | EFA1c0039_orf_26p | 10734 |
| E3M10000029G05 | 353 | EFA102351 | 4989 | EFA1c0032_orf_20p | 10634 |
| E3M10000029G07 | 354 | EFA101121 | 4912 | EFA1c0036_orf_112p | 10686 |
| E3M10000029G08 | 355 | EFA103571 | 5030 | EFA1c0044_orf_101p | 10879 |
| E3M10000029G09 | 356 | EFA102201 | 4982 | #N/A | #N/A |
| E3M10000029G10 | 357 | EFA101797 | 4963 | EFA1c0045_orf_167p | 10924 |
| E3M10000029G11 | 358 | EFA102006 | 4973 | EFA1c0025_orf_17p | 10580 |
| E3M10000029G12 | 359 | EFA101541 | 4948 | EFA1c0012_orf_5p | 10488 |
| E3M10000029H02 | 360 | EFA101339 | 4928 | EFA1c0040_orf_13p | 10743 |
| E3M10000029H02 | 360 | EFA101340 | 4929 | EFA1c0040_orf_15p | 10745 |
| E3M10000029H04 | 361 | EFA102352 | 4990 | EFA1c0032_orf_21p | 10635 |
| E3M10000029H04 | 361 | EFA102353 | 4991 | EFA1c0032_orf_22p | 10636 |
| E3M10000029H05 | 362 | EFA102091 | 4977 | EFA1c0010_orf_3p | 10481 |
| E3M10000029H07 | 363 | EFA100190 | 4867 | EFA1c0010_orf_2p | 10480 |
| E3M10000029H08 | 364 | EFA101416 | 4941 | EFA1c0022_orf_17p | 10530 |
| E3M10000029H11 | 365 | EFA101159 | 4916 | EFA1c0022_orf_2p | 10543 |
| E3M10000030A05 | 366 | EFA102501 | 4994 | EFA1c0031_orf_35p | 10626 |
| E3M10000030A08 | 367 | EFA102351 | 4989 | EFA1c0032_orf_20p | 10634 |
| E3M10000030A09 | 368 | EFA102501 | 4994 | EFA1c0031_orf_35p | 10626 |
| E3M10000030A11 | 369 | EFA102736 | 5007 | EFA1c0022_orf_60p | 10556 |
| E3M10000030B03 | 370 | EFA100704 | 4887 | EFA1c0010_orf_4p | 10482 |
| E3M10000030B04 | 371 | EFA103038 | 5017 | EFA1c0030_orf_17p | 10613 |
| E3M10000030B05 | 372 | EFA102656 | 5004 | EFA1c0039_orf_26p | 10734 |
| E3M10000030B06 | 373 | EFA101162 | 4919 | EFA1c0022_orf_5p | 10555 |
| E3M10000030B07 | 374 | EFA100642 | 4884 | EFA1c0041_orf_56p | 10792 |
| E3M10000030B08 | 375 | EFA102656 | 5004 | EFA1c0039_orf_26p | 10734 |
| E3M10000030B10 | 376 | EFA102655 | 5003 | EFA1c0039_orf_25p | 10733 |
| E3M10000030B11 | 377 | EFA101121 | 4912 | EFA1c0036_orf_112p | 10686 |
| E3M10000030B12 | 378 | EFA102352 | 4990 | EFA1c0032_orf_21p | 10635 |
| E3M10000030B12 | 378 | EFA102353 | 4991 | EFA1c0032_orf_22p | 10636 |
| E3M10000030C03 | 379 | EFA100151 | 4864 | EFA1c0021_orf_14p | 10516 |
| E3M10000030C04 | 380 | EFA101165 | 4922 | EFA1c0022_orf_8p | 10559 |
| E3M10000030C12 | 381 | EFA102351 | 4989 | EFA1c0032_orf_20p | 10634 |
| E3M10000030D02 | 382 | EFA102350 | 4988 | EFA1c0032_orf_19p | 10632 |
| E3M10000030D05 | 383 | EFA101414 | 4939 | EFA1c0022_orf_15p | 10528 |
| E3M10000030D08 | 384 | EFA102780 | 5010 | EFA1c0045_orf_101p | 10908 |
| E3M10000030D09 | 385 | EFA102780 | 5010 | EFA1c0045_orf_101p | 10908 |
| E3M10000030D10 | 386 | EFA102656 | 5004 | EFA1c0039_orf_26p | 10734 |
| E3M10000030D12 | 387 | EFA101417 | 4942 | EFA1c0022_orf_18p | 10531 |
| E3M10000030E01 | 388 | EFA101410 | 4935 | EFA1c0022_orf_12p | 10525 |
| E3M10000030E01 | 388 | EFA101411 | 4936 | EFA1c0022_orf_13p | 10526 |
| E3M10000030E02 | 389 | EFA100329 | 4875 | EFA1c0041_orf_35p | 10782 |
| E3M10000030E04 | 390 | EFA102655 | 5003 | EFA1c0039_orf_25p | 10733 |
| E3M10000030E08 | 391 | EFA101540 | 4947 | EFA1c0012_orf_4p | 10487 |
| E3M10000030E09 | 392 | EFA103365 | 5026 | EFA1c0022_orf_1p | 10533 |
| E3M10000030E10 | 393 | EFA102656 | 5004 | EFA1c0039_orf_26p | 10734 |
| E3M10000030F01 | 394 | EFA102655 | 5003 | EFA1c0039_orf_25p | 10733 |
| E3M10000030F04 | 395 | EFA101162 | 4919 | EFA1c0022_orf_5p | 10555 |
| E3M10000030F06 | 396 | EFA101162 | 4919 | EFA1c0022_orf_5p | 10555 |
| E3M10000030F07 | 397 | EFA102656 | 5004 | EFA1c0039_orf_26p | 10734 |
| E3M10000030F10 | 398 | EFA101417 | 4942 | EFA1c0022_orf_18p | 10531 |
| E3M10000030F12 | 399 | EFA102091 | 4977 | EFA1c0010_orf_3p | 10481 |
| E3M10000030G01 | 400 | EFA102551 | 5001 | EFA1c0022_orf_25p | 10539 |
| E3M10000030G03 | 401 | EFA100023 | 4862 | EFA1c0017_orf_1p | 10505 |
| E3M10000030G06 | 402 | EFA101686 | 4953 | EFA1c0045_orf_63p | 10940 |
| E3M10000030G08 | 403 | EFA102501 | 4994 | EFA1c0031_orf_35p | 10626 |
| E3M10000030G09 | 404 | EFA103210 | 5022 | EFA1c0036_orf_119p | 10688 |
| E3M10000030G12 | 405 | EFA103504 | 5028 | EFA1c0033_orf_94p | 10671 |
| E3M10000030H03 | 406 | EFA101258 | 4926 | EFA1c0045_orf_160p | 10918 |
| E3M10000030H04 | 407 | EFA101121 | 4912 | EFA1c0036_orf_112p | 10686 |
| E3M10000030H06 | 408 | EFA101161 | 4918 | EFA1c0022_orf_4p | 10551 |
| E3M10000030H07 | 409 | EFA101165 | 4922 | EFA1c0022_orf_8p | 10559 |
| E3M10000030H08 | 410 | EFA102501 | 4994 | EFA1c0031_orf_35p | 10626 |
| E3M10000030H10 | 411 | EFA102091 | 4977 | EFA1c0010_orf_3p | 10481 |
| E3M10000030H11 | 412 | EFA100615 | 4881 | EFA1c0016_orf_29p | 10501 |
| E3M10000031A02 | 413 | EFA102006 | 4973 | EFA1c0025_orf_17p | 10580 |
| E3M10000031A06 | 414 | EFA100970 | 4903 | EFA1c0044_orf_98p | 10906 |
| E3M10000031A07 | 415 | EFA102201 | 4982 | #N/A | #N/A |
| E3M10000031A08 | 416 | EFA100642 | 4884 | EFA1c0041_orf_56p | 10792 |
| E3M10000031B02 | 417 | EFA100289 | 4872 | EFA1c0042_orf_139p | 10810 |
| E3M10000031B03 | 418 | EFA100426 | 4879 | EFA1c0036_orf_59p | 10702 |
| E3M10000031B04 | 419 | EFA100394 | 4876 | EFA1c0034_orf_6p | 10675 |
| E3M10000031B09 | 420 | EFA102183 | 4979 | EFA1c0045_orf_97p | 10952 |
| E3M10000031B10 | 421 | EFA101253 | 4924 | EFA1c0043_orf_178p | 10852 |
| E3M10000031B11 | 422 | EFA100190 | 4867 | EFA1c0010_orf_2p | 10480 |
| E3M10000031B12 | 423 | EFA100642 | 4884 | EFA1c0041_orf_56p | 10792 |
| E3M10000031C01 | 424 | EFA102736 | 5007 | EFA1c0022_orf_60p | 10556 |
| E3M10000031C04 | 425 | EFA101414 | 4939 | EFA1c0022_orf_15p | 10528 |
| E3M10000031C06 | 426 | EFA100704 | 4887 | EFA1c0010_orf_4p | 10482 |
| E3M10000031C10 | 427 | EFA101411 | 4936 | EFA1c0022_orf_13p | 10526 |
| E3M10000031C11 | 428 | EFA101120 | 4911 | EFA1c0036_orf_113p | 10687 |
| E3M10000031C12 | 429 | EFA100668 | 4885 | EFA1c0035_orf_58p | 10679 |
| E3M10000031D03 | 430 | EFA102503 | 4996 | EFA1c0032_orf_32p | 10643 |
| E3M10000031D04 | 431 | EFA102502 | 4995 | EFA1c0031_orf_36p | 10627 |
| E3M10000031D08 | 432 | EFA102503 | 4996 | EFA1c0032_orf_32p | 10643 |
| E3M10000031E03 | 433 | EFA102501 | 4994 | EFA1c0031_orf_35p | 10626 |
| E3M10000031E09 | 434 | EFA102736 | 5007 | EFA1c0022_orf_60p | 10556 |
| E3M10000031F02 | 435 | EFA100642 | 4884 | EFA1c0041_orf_56p | 10792 |
| E3M10000031F02 | 435 | EFA101685 | 4952 | EFA1c0041_orf_55p | 10791 |
| E3M10000031F04 | 436 | EFA101160 | 4917 | EFA1c0022_orf_3p | 10549 |
| E3M10000031F07 | 437 | EFA102656 | 5004 | EFA1c0039_orf_26p | 10734 |
| E3M10000031F09 | 438 | EFA102764 | 5008 | EFA1c0008_orf_3p | 10478 |
| E3M10000031F11 | 439 | EFA102549 | 5000 | EFA1c0022_orf_24p | 10538 |
| E3M10000031F11 | 439 | EFA102551 | 5001 | EFA1c0022_orf_25p | 10539 |
| E3M10000031G03 | 440 | EFA102655 | 5003 | EFA1c0039_orf_25p | 10733 |
| E3M10000031G04 | 441 | EFA103571 | 5030 | EFA1c0044_orf_101p | 10879 |
| E3M10000031G05 | 442 | EFA102501 | 4994 | EFA1c0031_orf_35p | 10626 |
| E3M10000031G06 | 443 | EFA102656 | 5004 | EFA1c0039_orf_26p | 10734 |
| E3M10000031G07 | 444 | EFA103038 | 5017 | EFA1c0030_orf_17p | 10613 |
| E3M10000031G08 | 445 | EFA100295 | 4873 | EFA1c0021_orf_15p | 10517 |
| E3M10000031G11 | 446 | EFA101162 | 4919 | EFA1c0022_orf_5p | 10555 |
| E3M10000031H05 | 447 | EFA101121 | 4912 | EFA1c0036_orf_112p | 10686 |
| E3M10000031H06 | 448 | EFA101540 | 4947 | EFA1c0012_orf_4p | 10487 |
| E3M10000031H07 | 449 | EFA103038 | 5017 | EFA1c0030_orf_17p | 10613 |
| E3M10000031H08 | 450 | EFA102736 | 5007 | EFA1c0022_orf_60p | 10556 |
| E3M10000031H10 | 451 | EFA103038 | 5017 | EFA1c0030_orf_17p | 10613 |
| E3M10000031H11 | 452 | EFA100642 | 4884 | EFA1c0041_orf_56p | 10792 |
| E3M10000031H11 | 452 | EFA101685 | 4952 | EFA1c0041_orf_55p | 10791 |
| E3M10000032A02 | 453 | EFA100704 | 4887 | EFA1c0010_orf_4p | 10482 |
| E3M10000032A04 | 454 | EFA101670 | 4950 | EFA1c0019_orf_20p | 10511 |
| E3M10000032A06 | 455 | EFA101022 | 4906 | EFA1c0043_orf_69p | 10875 |
| E3M10000032A07 | 456 | EFA101670 | 4950 | EFA1c0019_orf_20p | 10511 |
| E3M10000032A08 | 457 | EFA100329 | 4875 | EFA1c0041_orf_35p | 10782 |
| E3M10000032A09 | 458 | EFA100394 | 4876 | EFA1c0034_orf_6p | 10675 |
| E3M10000032A10 | 459 | EFA101410 | 4935 | EFA1c0022_orf_12p | 10525 |
| E3M10000032A11 | 460 | EFA100642 | 4884 | EFA1c0041_orf_56p | 10792 |
| E3M10000032A11 | 460 | EFA101685 | 4952 | EFA1c0041_orf_55p | 10791 |
| E3M10000032B03 | 461 | EFA101540 | 4947 | EFA1c0012_orf_4p | 10487 |
| E3M10000032B04 | 462 | EFA102091 | 4977 | EFA1c0010_orf_3p | 10481 |
| E3M10000032B07 | 463 | EFA101164 | 4921 | EFA1c0022_orf_7p | 10558 |
| E3M10000032B08 | 464 | EFA102698 | 5005 | EFA1c0045_orf_115p | 10909 |
| E3M10000032B09 | 465 | EFA102051 | 4976 | #N/A | #N/A |
| E3M10000032B11 | 466 | EFA102091 | 4977 | EFA1c0010_orf_3p | 10481 |
| E3M10000032B12 | 467 | EFA100295 | 4873 | EFA1c0021_orf_15p | 10517 |
| E3M10000032C01 | 468 | EFA103062 | 5019 | EFA1c0030_orf_19p | 10615 |
| E3M10000032C02 | 469 | EFA100151 | 4864 | EFA1c0021_orf_14p | 10516 |
| E3M10000032C03 | 470 | EFA103348 | 5025 | EFA1c0043_orf_67p | 10873 |
| E3M10000032C04 | 471 | EFA101163 | 4920 | EFA1c0022_orf_6p | 10557 |
| E3M10000032C06 | 472 | EFA101150 | 4915 | EFA1c0038_orf_57p | 10719 |
| E3M10000032C09 | 473 | EFA100740 | 4889 | EFA1c0022_orf_22p | 10536 |
| E3M10000032C11 | 474 | EFA102501 | 4994 | EFA1c0031_orf_35p | 10626 |
| E3M10000032C12 | 475 | EFA101165 | 4922 | EFA1c0022_orf_8p | 10559 |
| E3M10000032D01 | 476 | EFA103504 | 5028 | EFA1c0033_orf_94p | 10671 |
| E3M10000032D02 | 477 | EFA101162 | 4919 | EFA1c0022_orf_5p | 10555 |
| E3M10000032D03 | 478 | EFA100399 | 4878 | EFA1c0041_orf_104p | 10766 |
| E3M10000032D06 | 479 | EFA100151 | 4864 | EFA1c0021_orf_14p | 10516 |
| E3M10000032D09 | 480 | EFA100151 | 4864 | EFA1c0021_orf_14p | 10516 |
| E3M10000032D12 | 481 | EFA101165 | 4922 | EFA1c0022_orf_8p | 10559 |
| E3M10000032E04 | 482 | EFA101792 | 4961 | EFA1c0042_orf_113p | 10805 |
| E3M10000032E04 | 482 | EFA103786 | 5031 | EFA1c0042_orf_114p | 10806 |
| E3M10000032E05 | 483 | EFA103038 | 5017 | EFA1c0030_orf_17p | 10613 |
| E3M10000032E08 | 484 | EFA101164 | 4921 | EFA1c0022_orf_7p | 10558 |
| E3M10000032E10 | 485 | EFA100870 | 4899 | EFA1c0031_orf_36p | 10627 |
| E3M10000032E10 | 485 | EFA102502 | 4995 | EFA1c0031_orf_36p | 10627 |
| E3M10000032E11 | 486 | EFA101414 | 4939 | EFA1c0022_orf_15p | 10528 |
| E3M10000032E12 | 487 | EFA102326 | 4986 | #N/A | #N/A |
| E3M10000032F02 | 488 | EFA100210 | 4870 | EFA1c0022_orf_9p | 10560 |
| E3M10000032F02 | 488 | EFA101165 | 4922 | EFA1c0022_orf_8p | 10559 |
| E3M10000032F03 | 489 | EFA101414 | 4939 | EFA1c0022_orf_15p | 10528 |
| E3M10000032F05 | 490 | EFA102541 | 4998 | EFA1c0028_orf_3p | 10602 |
| E3M10000032F07 | 491 | EFA102780 | 5010 | EFA1c0045_orf_101p | 10908 |
| E3M10000032F08 | 492 | EFA102501 | 4994 | EFA1c0031_orf_35p | 10626 |
| E3M10000032F11 | 493 | EFA100642 | 4884 | EFA1c0041_orf_56p | 10792 |
| E3M10000032F12 | 494 | EFA102201 | 4982 | #N/A | #N/A |
| E3M10000032G01 | 495 | EFA102502 | 4995 | EFA1c0031_orf_36p | 10627 |
| E3M10000032G02 | 496 | EFA100870 | 4899 | EFA1c0031_orf_36p | 10627 |
| E3M10000032G04 | 497 | EFA100704 | 4887 | EFA1c0010_orf_4p | 10482 |
| E3M10000032G05 | 498 | EFA101540 | 4947 | EFA1c0012_orf_4p | 10487 |
| E3M10000032G06 | 499 | EFA100190 | 4867 | EFA1c0010_orf_2p | 10480 |
| E3M10000032G07 | 500 | EFA100919 | 4901 | EFA1c0013_orf_12p | 10491 |
| E3M10000032H05 | 501 | EFA100200 | 4869 | EFA1c0041_orf_88p | 10798 |
| E3M10000032H06 | 502 | EFA101833 | 4965 | EFA1c0038_orf_62p | 10720 |
| E3M10000032H08 | 503 | EFA102656 | 5004 | EFA1c0039_orf_26p | 10734 |
| E3M10000032H09 | 504 | EFA103571 | 5030 | EFA1c0044_orf_101p | 10879 |
| E3M10000032H10 | 505 | EFA100642 | 4884 | EFA1c0041_orf_56p | 10792 |
| E3M10000033A03 | 506 | EFA101253 | 4924 | EFA1c0043_orf_178p | 10852 |
| E3M10000033A04 | 507 | EFA102503 | 4996 | EFA1c0032_orf_32p | 10643 |
| E3M10000033A05 | 508 | EFA102551 | 5001 | EFA1c0022_orf_25p | 10539 |
| E3M10000033A06 | 509 | EFA101415 | 4940 | EFA1c0022_orf_16p | 10529 |
| E3M10000033A07 | 510 | EFA102774 | 5009 | EFA1c0044_orf_25p | 10896 |
| E3M10000033A08 | 511 | EFA102656 | 5004 | EFA1c0039_orf_26p | 10734 |
| E3M10000033A11 | 512 | EFA100642 | 4884 | EFA1c0041_orf_56p | 10792 |
| E3M10000033B01 | 513 | EFA102006 | 4973 | EFA1c0025_orf_17p | 10580 |
| E3M10000033B02 | 514 | EFA101412 | 4937 | EFA1c0022_orf_14p | 10527 |
| E3M10000033B04 | 515 | EFA101765 | 4958 | EFA1c0025_orf_33p | 10587 |
| E3M10000033B05 | 516 | EFA102541 | 4998 | EFA1c0028_orf_3p | 10602 |
| E3M10000033B06 | 517 | EFA102351 | 4989 | EFA1c0032_orf_20p | 10634 |
| E3M10000033B08 | 518 | EFA102091 | 4977 | EFA1c0010_orf_3p | 10481 |
| E3M10000033B09 | 519 | EFA100210 | 4870 | EFA1c0022_orf_9p | 10560 |
| E3M10000033C01 | 520 | EFA101540 | 4947 | EFA1c0012_orf_4p | 10487 |
| E3M10000033C02 | 521 | EFA103174 | 5021 | EFA1c0036_orf_120p | 10689 |
| E3M10000033C05 | 522 | EFA102541 | 4998 | EFA1c0028_orf_3p | 10602 |
| E3M10000033C05 | 522 | EFA102542 | 4999 | EFA1c0028_orf_4p | 10603 |
| E3M10000033C09 | 523 | EFA100811 | 4898 | EFA1c0022_orf_33p | 10546 |
| E3M10000033C10 | 524 | EFA101410 | 4935 | EFA1c0022_orf_12p | 10525 |
| E3M10000033C10 | 524 | EFA101411 | 4936 | EFA1c0022_orf_13p | 10526 |
| E3M10000033C11 | 525 | EFA103504 | 5028 | EFA1c0033_orf_94p | 10671 |
| E3M10000033C12 | 526 | EFA102389 | 4992 | EFA1c0044_orf_83p | 10904 |
| E3M10000033D01 | 527 | EFA102351 | 4989 | EFA1c0032_orf_20p | 10634 |
| E3M10000033D04 | 528 | EFA101682 | 4951 | EFA1c0041_orf_53p | 10789 |
| E3M10000033D05 | 529 | EFA101417 | 4942 | EFA1c0022_orf_18p | 10531 |
| E3M10000033D06 | 530 | EFA100641 | 4883 | EFA1c0041_orf_57p | 10793 |
| E3M10000033D06 | 530 | EFA100642 | 4884 | EFA1c0041_orf_56p | 10792 |
| E3M10000033D09 | 531 | EFA101414 | 4939 | EFA1c0022_orf_15p | 10528 |
| E3M10000033D10 | 532 | EFA102006 | 4973 | EFA1c0025_orf_17p | 10580 |
| E3M10000033D11 | 533 | EFA102091 | 4977 | EFA1c0010_orf_3p | 10481 |
| E3M10000033E02 | 534 | EFA101477 | 4945 | EFA1c0043_orf_224p | 10861 |
| E3M10000033E03 | 535 | EFA101414 | 4939 | EFA1c0022_orf_15p | 10528 |
| E3M10000033E03 | 535 | EFA101415 | 4940 | EFA1c0022_orf_16p | 10529 |
| E3M10000033E04 | 536 | EFA102656 | 5004 | EFA1c0039_orf_26p | 10734 |
| E3M10000033E05 | 537 | EFA102503 | 4996 | EFA1c0032_orf_32p | 10643 |
| E3M10000033E07 | 538 | EFA102502 | 4995 | EFA1c0031_orf_36p | 10627 |
| E3M10000033E08 | 539 | EFA102351 | 4989 | EFA1c0032_orf_20p | 10634 |
| E3M10000033E09 | 540 | EFA100617 | 4882 | EFA1c0040_orf_93p | 10764 |
| E3M10000033E11 | 541 | EFA102551 | 5001 | EFA1c0022_orf_25p | 10539 |
| E3M10000033F01 | 542 | EFA102502 | 4995 | EFA1c0031_orf_36p | 10627 |
| E3M10000033F03 | 543 | EFA101686 | 4953 | EFA1c0045_orf_63p | 10940 |
| E3M10000033F04 | 544 | EFA100704 | 4887 | EFA1c0010_orf_4p | 10482 |
| E3M10000033F05 | 545 | EFA102501 | 4994 | EFA1c0031_orf_35p | 10626 |
| E3M10000033F07 | 546 | EFA102502 | 4995 | EFA1c0031_orf_36p | 10627 |
| E3M10000033F08 | 547 | EFA101165 | 4922 | EFA1c0022_orf_8p | 10559 |
| E3M10000033F10 | 548 | EFA103571 | 5030 | EFA1c0044_orf_101p | 10879 |
| E3M10000033F12 | 549 | EFA102541 | 4998 | EFA1c0028_orf_3p | 10602 |
| E3M10000033F12 | 549 | EFA102542 | 4999 | EFA1c0028_orf_4p | 10603 |
| E3M10000033G01 | 550 | EFA101163 | 4920 | EFA1c0022_orf_6p | 10557 |
| E3M10000033G02 | 551 | EFA102813 | 5013 | EFA1c0043_orf_9p | 10878 |
| E3M10000033G03 | 552 | EFA102656 | 5004 | EFA1c0039_orf_26p | 10734 |
| E3M10000033G04 | 553 | EFA102326 | 4986 | #N/A | #N/A |
| E3M10000033G06 | 554 | EFA101404 | 4933 | EFA1c0033_orf_55p | 10663 |
| E3M10000033G07 | 555 | EFA101685 | 4952 | EFA1c0041_orf_55p | 10791 |
| E3M10000033G08 | 556 | EFA101141 | 4914 | EFA1c0030_orf_18p | 10614 |
| E3M10000033G09 | 557 | EFA102656 | 5004 | EFA1c0039_orf_26p | 10734 |
| E3M10000033G12 | 558 | EFA101686 | 4953 | EFA1c0045_orf_63p | 10940 |
| E3M10000033H02 | 559 | EFA101415 | 4940 | EFA1c0022_orf_16p | 10529 |
| E3M10000033H04 | 560 | EFA102780 | 5010 | EFA1c0045_orf_101p | 10908 |
| E3M10000033H05 | 561 | EFA100741 | 4890 | EFA1c0022_orf_21p | 10535 |
| E3M10000033H07 | 562 | EFA102502 | 4995 | EFA1c0031_orf_36p | 10627 |
| E3M10000033H08 | 563 | EFA101160 | 4917 | EFA1c0022_orf_3p | 10549 |
| E3M10000033H09 | 564 | EFA100642 | 4884 | EFA1c0041_orf_56p | 10792 |
| E3M10000033H10 | 565 | EFA101079 | 4908 | #N/A | #N/A |
| E3M10000033H11 | 566 | EFA100190 | 4867 | EFA1c0010_orf_2p | 10480 |
| E3M10000034A02 | 567 | EFA102501 | 4994 | EFA1c0031_orf_35p | 10626 |
| E3M10000034A03 | 568 | EFA100978 | 4904 | EFA1c0022_orf_27p | 10541 |
| E3M10000034A04 | 569 | EFA103038 | 5017 | EFA1c0030_orf_17p | 10613 |
| E3M10000034B02 | 570 | EFA103504 | 5028 | EFA1c0033_orf_94p | 10671 |
| E3M10000034B04 | 571 | EFA102655 | 5003 | EFA1c0039_orf_25p | 10733 |
| E3M10000034C04 | 572 | EFA102502 | 4995 | EFA1c0031_orf_36p | 10627 |
| E3M10000034D01 | 573 | EFA100704 | 4887 | EFA1c0010_orf_4p | 10482 |
| E3M10000034D02 | 574 | EFA100190 | 4867 | EFA1c0010_orf_2p | 10480 |
| E3M10000034E01 | 575 | EFA101162 | 4919 | EFA1c0022_orf_5p | 10555 |
| E3M10000034E04 | 576 | EFA100190 | 4867 | EFA1c0010_orf_2p | 10480 |
| E3M10000034F02 | 577 | EFA101162 | 4919 | EFA1c0022_orf_5p | 10555 |
| E3M10000034F03 | 578 | EFA103038 | 5017 | EFA1c0030_orf_17p | 10613 |
| E3M10000034F04 | 579 | EFA100190 | 4867 | EFA1c0010_orf_2p | 10480 |
| E3M10000034G02 | 580 | EFA102501 | 4994 | EFA1c0031_orf_35p | 10626 |
| E3M10000034G03 | 581 | EFA100740 | 4889 | EFA1c0022_orf_22p | 10536 |
| E3M10000034H02 | 582 | EFA101257 | 4925 | EFA1c0045_orf_159p | 10917 |
| E3M10000034H03 | 583 | EFA102501 | 4994 | EFA1c0031_orf_35p | 10626 |
| E3M10000035A02 | 584 | EFA103268 | 5023 | EFA1c0010_orf_1p | 10479 |
| E3M10000035A04 | 585 | EFA103571 | 5030 | EFA1c0044_orf_101p | 10879 |
| E3M10000035A05 | 586 | EFA101540 | 4947 | EFA1c0012_orf_4p | 10487 |
| E3M10000035A06 | 587 | EFA103571 | 5030 | EFA1c0044_orf_101p | 10879 |
| E3M10000035A08 | 588 | EFA103038 | 5017 | EFA1c0030_orf_17p | 10613 |
| E3M10000035A09 | 589 | EFA100210 | 4870 | EFA1c0022_orf_9p | 10560 |
| E3M10000035A11 | 590 | EFA100151 | 4864 | EFA1c0021_orf_14p | 10516 |
| E3M10000035B01 | 591 | EFA101022 | 4906 | EFA1c0043_orf_69p | 10875 |
| E3M10000035B03 | 592 | EFA100704 | 4887 | EFA1c0010_orf_4p | 10482 |
| E3M10000035B06 | 593 | EFA101164 | 4921 | EFA1c0022_orf_7p | 10558 |
| E3M10000035B07 | 594 | EFA103571 | 5030 | EFA1c0044_orf_101p | 10879 |
| E3M10000035B08 | 595 | EFA102780 | 5010 | EFA1c0045_orf_101p | 10908 |
| E3M10000035B10 | 596 | EFA100151 | 4864 | EFA1c0021_orf_14p | 10516 |
| E3M10000035B11 | 597 | EFA103571 | 5030 | EFA1c0044_orf_101p | 10879 |
| E3M10000035B12 | 598 | EFA103038 | 5017 | EFA1c0030_orf_17p | 10613 |
| E3M10000035C01 | 599 | EFA100704 | 4887 | EFA1c0010_orf_4p | 10482 |
| E3M10000035C03 | 600 | EFA101417 | 4942 | EFA1c0022_orf_18p | 10531 |
| E3M10000035C04 | 601 | EFA103038 | 5017 | EFA1c0030_orf_17p | 10613 |
| E3M10000035C05 | 602 | EFA100870 | 4899 | EFA1c0031_orf_36p | 10627 |
| E3M10000035C06 | 603 | EFA101160 | 4917 | EFA1c0022_orf_3p | 10549 |
| E3M10000035C07 | 604 | EFA100870 | 4899 | EFA1c0031_orf_36p | 10627 |
| E3M10000035C08 | 605 | EFA100741 | 4890 | EFA1c0022_orf_21p | 10535 |
| E3M10000035C08 | 605 | EFA100742 | 4891 | EFA1c0022_orf_20p | 10534 |
| E3M10000035C09 | 606 | EFA103062 | 5019 | EFA1c0030_orf_19p | 10615 |
| E3M10000035C11 | 607 | EFA100704 | 4887 | EFA1c0010_orf_4p | 10482 |
| E3M10000035C12 | 608 | EFA100704 | 4887 | EFA1c0010_orf_4p | 10482 |
| E3M10000035D02 | 609 | EFA101160 | 4917 | EFA1c0022_orf_3p | 10549 |
| E3M10000035D03 | 610 | EFA103504 | 5028 | EFA1c0033_orf_94p | 10671 |
| E3M10000035D04 | 611 | EFA101540 | 4947 | EFA1c0012_orf_4p | 10487 |
| E3M10000035D05 | 612 | EFA101162 | 4919 | EFA1c0022_orf_5p | 10555 |
| E3M10000035D10 | 613 | EFA103571 | 5030 | EFA1c0044_orf_101p | 10879 |
| E3M10000035D11 | 614 | EFA100919 | 4901 | EFA1c0013_orf_12p | 10491 |
| E3M10000035E03 | 615 | EFA101414 | 4939 | EFA1c0022_orf_15p | 10528 |
| E3M10000035E04 | 616 | EFA101141 | 4914 | EFA1c0030_orf_18p | 10614 |
| E3M10000035E05 | 617 | EFA102006 | 4973 | EFA1c0025_orf_17p | 10580 |
| E3M10000035E07 | 618 | EFA100919 | 4901 | EFA1c0013_orf_12p | 10491 |
| E3M10000035E08 | 619 | EFA101162 | 4919 | EFA1c0022_orf_5p | 10555 |
| E3M10000035E09 | 620 | EFA100312 | 4874 | EFA1c0032_orf_28p | 10641 |
| E3M10000035E10 | 621 | EFA101022 | 4906 | EFA1c0043_orf_69p | 10875 |
| E3M10000035E11 | 622 | EFA100870 | 4899 | EFA1c0031_orf_36p | 10627 |
| E3M10000035E12 | 623 | EFA100704 | 4887 | EFA1c0010_orf_4p | 10482 |
| E3M10000035F01 | 624 | EFA101417 | 4942 | EFA1c0022_orf_18p | 10531 |
| E3M10000035F02 | 625 | EFA101925 | 4971 | EFA1c0044_orf_19p | 10893 |
| E3M10000035F03 | 626 | EFA100312 | 4874 | EFA1c0032_orf_28p | 10641 |
| E3M10000035F06 | 627 | EFA101080 | 4909 | #N/A | #N/A |
| E3M10000035F07 | 628 | EFA101165 | 4922 | EFA1c0022_orf_8p | 10559 |
| E3M10000035F08 | 629 | EFA100704 | 4887 | EFA1c0010_orf_4p | 10482 |
| E3M10000035F09 | 630 | EFA101410 | 4935 | EFA1c0022_orf_12p | 10525 |
| E3M10000035F09 | 630 | EFA101411 | 4936 | EFA1c0022_orf_13p | 10526 |
| E3M10000035F11 | 631 | EFA100704 | 4887 | EFA1c0010_orf_4p | 10482 |
| E3M10000035F12 | 632 | EFA101120 | 4911 | EFA1c0036_orf_113p | 10687 |
| E3M10000035G02 | 633 | EFA100190 | 4867 | EFA1c0010_orf_2p | 10480 |
| E3M10000035G02 | 633 | EFA102091 | 4977 | EFA1c0010_orf_3p | 10481 |
| E3M10000035G04 | 634 | EFA100210 | 4870 | EFA1c0022_orf_9p | 10560 |
| E3M10000035G05 | 635 | EFA102502 | 4995 | EFA1c0031_orf_36p | 10627 |
| E3M10000035G08 | 636 | EFA100642 | 4884 | EFA1c00241_orf_56p | 10792 |
| E3M10000035G09 | 637 | EFA103504 | 5028 | EFA1c0033_orf_94p | 10671 |
| E3M10000035G09 | 637 | EFA103508 | 5029 | EFA1c0033_orf_95p | 10672 |
| E3M10000035G10 | 638 | EFA103038 | 5017 | EFA1c0030_orf_17p | 10613 |
| E3M10000035G11 | 639 | EFA101540 | 4947 | EFA1c0012_orf_4p | 10487 |
| E3M10000035H03 | 640 | EFA101080 | 4909 | #N/A | #N/A |
| E3M10000035H06 | 641 | EFA100210 | 4870 | EFA1c0022_orf_9p | 10560 |
| E3M10000035H09 | 642 | EFA102501 | 4994 | EFA1c0031_orf_35p | 10626 |
| E3M10000035H11 | 643 | EFA101257 | 4925 | EFA1c0045_orf_159p | 10917 |
| E3M10000035H11 | 643 | EFA101258 | 4926 | EFA1c0045_orf_160p | 10918 |
| E3M10000036A03 | 644 | EFA103504 | 5028 | EFA1c0033_orf_94p | 10671 |
| E3M10000036A04 | 645 | EFA101416 | 4941 | EFA1c0022_orf_17p | 10530 |
| B3M10000036A05 | 646 | EFA102780 | 5010 | EFA1c0045_orf_101p | 10908 |
| E3M10000036A06 | 647 | EFA101540 | 4947 | EFA1c0012_orf_4p | 10487 |
| E3M10000036A07 | 648 | EFA103268 | 5023 | EFA1c0010_orf_1p | 10479 |
| E3M10000036A08 | 649 | EFA103038 | 5017 | EFA1c0030_orf_17p | 10613 |
| E3M10000036A09 | 650 | EFA101165 | 4922 | EFA1c0022_orf_8p | 10559 |
| E3M10000036A10 | 651 | EFA100210 | 4870 | EFA1c0022_orf_9p | 10560 |
| E3M10000036B01 | 652 | EFA101121 | 4912 | EFA1c0036_orf_112p | 10686 |
| E3M10000036B03 | 653 | EFA101686 | 4953 | EFA1c0045_orf_63p | 10940 |
| E3M10000036B06 | 654 | EFA101162 | 4919 | EFA1c0022_orf_5p | 10555 |
| E3M10000036B07 | 655 | EFA103038 | 5017 | EFA1c0030_orf_17p | 10613 |
| E3M10000036B08 | 656 | EFA100151 | 4864 | EFA1c0021_orf_14p | 10516 |
| E3M10000036B09 | 657 | EFA100190 | 4867 | EFA1c0010_orf_2p | 10480 |
| E3M10000036B11 | 658 | EFA103504 | 5028 | EFA1c0033_orf_94p | 10671 |
| E3M10000036B12 | 659 | EFA101162 | 4919 | EFA1c0022_orf_5p | 10555 |
| E3M10000036B12 | 659 | EFA101163 | 4920 | EFA1c0022_orf_6p | 10557 |
| E3M10000036C01 | 660 | EFA101416 | 4941 | EFA1c0022_orf_17p | 10530 |
| E3M10000036C03 | 661 | EFA103571 | 5030 | EFA1c0044_orf_101p | 10879 |
| E3M10000036C06 | 662 | EFA102091 | 4977 | EFA1c0010_orf_3p | 10481 |
| E3M10000036C07 | 663 | EFA101141 | 4914 | EFA1c0030_orf_18p | 10614 |
| E3M10000036C08 | 664 | EFA100151 | 4864 | EFA1c0021_orf_14p | 10516 |
| E3M10000036C09 | 665 | EFA101540 | 4947 | EFA1c0012_orf_4p | 10487 |
| E3M10000036C10 | 666 | EFA101540 | 4947 | EFA1c0012_orf_4p | 10487 |
| E3M10000036C11 | 667 | EFA101417 | 4942 | EFA1c0022_orf_18p | 10531 |
| E3M10000036D03 | 668 | EFA100704 | 4887 | EFA1c0010_orf_4p | 10482 |
| E3M10000036D04 | 669 | EFA102201 | 4982 | #N/A | #N/A |
| E3M10000036D06 | 670 | EFA100740 | 4889 | EFA1c0022_orf_22p | 10536 |
| E3M10000036D08 | 671 | EFA101164 | 4921 | EFA1c0022_orf_7p | 10558 |
| E3M10000036D09 | 672 | EFA103571 | 5030 | EFA1c0044_orf_101p | 10879 |
| E3M10000036D10 | 673 | EFA101121 | 4912 | EFA1c0036_orf_112p | 10686 |
| E3M10000036D11 | 674 | EFA102502 | 4995 | EFA1c0031_orf_36p | 10627 |
| E3M10000036D12 | 675 | EFA102091 | 4977 | EFA1c0010_orf_3p | 10481 |
| E3M10000036E01 | 676 | EFA101121 | 4912 | EFA1c0036_orf_112p | 10686 |
| E3M10000036E04 | 677 | EFA101162 | 4919 | EFA1c0022_orf_5p | 10555 |
| E3M10000036E05 | 678 | EFA101414 | 4939 | EFA1c0022_orf_15p | 10528 |
| E3M10000036E07 | 679 | EFA101022 | 4906 | EFA1c0043_orf_69p | 10875 |
| E3M10000036E08 | 680 | EFA100210 | 4870 | EFA1c0022_orf_9p | 10560 |
| E3M10000036F03 | 681 | EFA100210 | 4870 | EFA1c0022_orf_9p | 10560 |
| E3M10000036F04 | 682 | EFA101686 | 4953 | EFA1c0045_orf_63p | 10940 |
| E3M10000036F05 | 683 | EFA101792 | 4961 | EFA1c0042_orf_113p | 10805 |
| E3M10000036F08 | 684 | EFA102091 | 4977 | EFA1c0010_orf_3p | 10481 |
| E3M10000036F09 | 685 | EFA101404 | 4933 | EFA1c0033_orf_55p | 10663 |
| E3M10000036F10 | 686 | EFA101162 | 4919 | EFA1c0022_orf_5p | 10555 |
| E3M10000036F12 | 687 | EFA101163 | 4920 | EFA1c0022_orf_6p | 10557 |
| E3M10000036G01 | 688 | EFA102549 | 5000 | EFA1c0022_orf_24p | 10538 |
| E3M10000036G01 | 688 | EFA102551 | 5001 | EFA1c0022_orf_25p | 10539 |
| E3M10000036G02 | 689 | EFA101121 | 4912 | EFA1c0036_orf_112p | 10686 |
| E3M10000036G03 | 690 | EFA102656 | 5004 | EFA1c0039_orf_26p | 10734 |
| E3M10000036G04 | 691 | EFA102091 | 4977 | EFA1c0010_orf_3p | 10481 |
| B3M10000036G06 | 692 | EFA100295 | 4873 | EFA1c0021_orf_15p | 10517 |
| E3M10000036G10 | 693 | EFA101162 | 4919 | EFA1c0022_orf_5p | 10555 |
| E3M10000036H02 | 694 | EFA103038 | 5017 | EFA1c0030_orf_17p | 10613 |
| E3M10000036H03 | 695 | EFA103571 | 5030 | EFA1c0044_orf_101p | 10879 |
| E3M10000036H04 | 696 | EFA103365 | 5026 | EFA1c0022_orf_1p | 10533 |
| E3M10000036H05 | 697 | EFA100194 | 4868 | EFA1c0022_orf_26p | 10540 |
| E3M10000036H06 | 698 | EFA101162 | 4919 | EFA1c0022_orf_5p | 10555 |
| E3M10000036H07 | 699 | EFA102501 | 4994 | EFA1c0031_orf_35p | 10626 |
| E3M10000036H08 | 700 | EFA103210 | 5022 | EFA1c0036_orf_119p | 10688 |
| E3M10000036H09 | 701 | EFA101162 | 4919 | EFA1c0022_orf_5p | 10555 |
| E3M10000036H10 | 702 | EFA101141 | 4914 | EFA1c0030_orf_18p | 10614 |
| E3M10000037A03 | 703 | EFA102091 | 4977 | EFA1c0010_orf_3p | 10481 |
| E3M10000037A06 | 704 | EFA100870 | 4899 | EFA1c0031_orf_36p | 10627 |
| E3M10000037A08 | 705 | EFA103365 | 5026 | EFA1c0022_orf_1p | 10533 |
| E3M10000037A09 | 706 | EFA100756 | 4893 | EFA1c0024_orf_39p | 10575 |
| E3M10000037A10 | 707 | EFA103268 | 5023 | EFA1c0010_orf_1p | 10479 |
| E3M10000037B02 | 708 | EFA100641 | 4883 | EFA1c0041_orf_57p | 10793 |
| E3M10000037B02 | 708 | EFA100642 | 4884 | EFA1c0041_orf_56p | 10792 |
| E3M10000037B07 | 709 | EFA101162 | 4919 | EFA1c0022_orf_5p | 10555 |
| E3M10000037B08 | 710 | EFA100151 | 4864 | EFA1c0021_orf_14p | 10516 |
| E3M10000037B11 | 711 | EFA101686 | 4953 | EFA1c0045_orf_63p | 10940 |
| E3M10000037C01 | 712 | EFA101080 | 4909 | #N/A | #N/A |
| E3M10000037C02 | 713 | EFA102351 | 4989 | EFA1c0032_orf_20p | 10634 |
| E3M10000037C04 | 714 | EFA103504 | 5028 | EFA1c0033_orf_94p | 10671 |
| E3M10000037C05 | 715 | EFA102655 | 5003 | EFA1c0039_orf_25p | 10733 |
| B3M10000037C07 | 716 | EFA101160 | 4917 | EFA1c0022_orf_3p | 10549 |
| E3M10000037C07 | 716 | EFA101161 | 4918 | EFA1c0022_orf_4p | 10551 |
| E3M10000037C11 | 717 | EFA100615 | 4881 | EFA1c0016_orf_29p | 10501 |
| E3M10000037C12 | 718 | EFA102502 | 4995 | EFA1c0031_orf_36p | 10627 |
| E3M10000037D02 | 719 | EFA100642 | 4884 | EFA1c0041_orf_56p | 10792 |
| E3M10000037D03 | 720 | EFA100795 | 4896 | EFA1c0043_orf_229p | 10863 |
| E3M10000037D03 | 720 | EFA103081 | 5020 | EFA1c0043_orf_28p | 10862 |
| E3M10000037D04 | 721 | EFA100210 | 4870 | EFA1c0022_orf_9p | 10560 |
| E3M10000037D05 | 722 | EFA101416 | 4941 | EFA1c0022_orf_17p | 10530 |
| E3M10000037D06 | 723 | EFA101161 | 4918 | EFA1c0022_orf_4p | 10551 |
| E3M10000037D09 | 724 | EFA100190 | 4867 | EFA1c0010_orf_2p | 10480 |
| E3M10000037D09 | 724 | EFA102091 | 4977 | EFA1c0010_orf_3p | 10481 |
| E3M10000037D11 | 725 | EFA100210 | 4870 | EFA1c0022_orf_9p | 10560 |
| E3M10000037E01 | 726 | EFA102736 | 5007 | EFA1c0022_orf_60p | 10556 |
| E3M10000037E02 | 727 | EFA100704 | 4887 | EFA1c0010_orf_4p | 10482 |
| E3M10000037E03 | 728 | EFA102503 | 4996 | EFA1c0032_orf_32p | 10643 |
| E3M10000037E05 | 729 | EFA101080 | 4909 | #N/A | #N/A |
| E3M10000037E07 | 730 | EFA100210 | 4870 | EFA1c0022_orf_9p | 10560 |
| E3M10000037E08 | 731 | EFA100642 | 4884 | EFA1c0041_orf_56p | 10792 |
| E3M10000037E10 | 732 | EFA101253 | 4924 | EFA1c0043_orf_178p | 10852 |
| E3M10000037E12 | 733 | EFA101686 | 4953 | EFA1c0045_orf_63p | 10940 |
| E3M10000037F01 | 734 | EFA103504 | 5028 | EFA1c0033_orf_94p | 10671 |
| E3M10000037F02 | 735 | EFA101160 | 4917 | EFA1c0022_orf_3p | 10549 |
| E3M10000037F06 | 736 | EFA100210 | 4870 | EFA1c0022_orf_9p | 10560 |
| E3M10000037F07 | 737 | EFA100210 | 4870 | EFA1c0022_orf_9p | 10560 |
| E3M10000037F12 | 738 | EFA101161 | 4918 | EFA1c0022_orf_4p | 10551 |
| E3M10000037G01 | 739 | EFA102656 | 5004 | EFA1c0039_orf_26p | 10734 |
| E3M10000037G02 | 740 | EFA101165 | 4922 | EFA1c0022_orf_8p | 10559 |
| E3M10000037G03 | 741 | EFA102780 | 5010 | EFA1c0045_orf_101p | 10908 |
| E3M10000037G05 | 742 | EFA102780 | 5010 | EFA1c0045_orf_101p | 10908 |
| E3M10000037G06 | 743 | EFA103295 | 5024 | EFA1c0032_orf_1p | 10633 |
| E3M10000037G07 | 744 | EFA101541 | 4948 | EFA1c0012_orf_5p | 10488 |
| E3M10000037G08 | 745 | EFA101121 | 4912 | EFA1c0036_orf_112p | 10686 |
| E3M10000037G10 | 746 | EFA101412 | 4937 | EFA1c0022_orf_14p | 10527 |
| E3M10000037G11 | 747 | EFA103038 | 5017 | EFA1c0030_orf_17p | 10613 |
| E3M10000037H02 | 748 | EFA101413 | 4938 | #N/A | #N/A |
| E3M10000037H05 | 749 | EFA101686 | 4953 | EFA1c0045_orf_63p | 10940 |
| E3M10000037H07 | 750 | EFA100955 | 4902 | EFA1c0022_orf_28p | 10542 |
| E3M10000037H10 | 751 | EFA101080 | 4909 | #N/A | #N/A |
| E3M10000037H11 | 752 | EFA102541 | 4998 | EFA1c0028_orf_3p | 10602 |
| E3M10000038A02 | 753 | EFA102541 | 4998 | EFA1c0028_orf_3p | 10602 |
| E3M10000038A03 | 754 | EFA103038 | 5017 | EFA1c0030_orf_17p | 10613 |
| E3M10000038A05 | 755 | EFA100151 | 4864 | EFA1c0021_orf_14p | 10516 |
| E3M10000038A06 | 756 | EFA102549 | 5000 | EFA1c0022_orf_24p | 10538 |
| E3M10000038A07 | 757 | EFA102501 | 4994 | EFA1c0031_orf_35p | 10626 |
| E3M10000038A09 | 758 | EFA102736 | 5007 | EFA1c0022_orf_60p | 10556 |
| E3M10000038A10 | 759 | EFA100210 | 4870 | EFA1c0022_orf_9p | 10560 |
| E3M10000038A11 | 760 | EFA101417 | 4942 | EFA1c0022_orf_18p | 10531 |
| E3M10000038B02 | 761 | EFA103210 | 5022 | EFA1c0036_orf_119p | 10688 |
| E3M10000038B03 | 762 | EFA102389 | 4992 | EFA1c0044_orf_83p | 10904 |
| E3M10000038B04 | 763 | EFA101414 | 4939 | EFA1c0022_orf_15p | 10528 |
| E3M10000038B05 | 764 | EFA100795 | 4896 | EFA1c0043_orf_229p | 10863 |
| E3M10000038B05 | 764 | EFA103081 | 5020 | EFA1c0043_orf_28p | 10862 |
| E3M10000038B07 | 765 | EFA100190 | 4867 | EFA1c0010_orf_2p | 10480 |
| E3M10000038B08 | 766 | EFA101160 | 4917 | EFA1c0022_orf_3p | 10549 |
| E3M10000038B09 | 767 | EFA101685 | 4952 | EFA1c0041_orf_55p | 10791 |
| E3M10000038B11 | 768 | EFA102656 | 5004 | EFA1c0039_orf_26p | 10734 |
| E3M10000038C02 | 769 | EFA102780 | 5010 | EFA1c0045_orf_101p | 10908 |
| E3M10000038C03 | 770 | EFA102656 | 5004 | EFA1c0039_orf_26p | 10734 |
| E3M10000038C05 | 771 | EFA101686 | 4953 | EFA1c0045_orf_63p | 10940 |
| E3M10000038C07 | 772 | EFA101963 | 4972 | EFA1c0043_orf_162p | 10848 |
| E3M10000038C10 | 773 | EFA102655 | 5003 | EFA1c0039_orf_25p | 10733 |
| E3M10000038C12 | 774 | EFA101080 | 4909 | #N/A | #N/A |
| E3M10000038D01 | 775 | EFA101540 | 4947 | EFA1c0012_orf_4p | 10487 |
| E3M10000038D02 | 776 | EFA103504 | 5028 | EFA1c0033_orf_94p | 10671 |
| E3M10000038D04 | 777 | EFA101540 | 4947 | EFA1c0012_orf_4p | 10487 |
| E3M10000038D08 | 778 | EFA101160 | 4917 | EFA1c0022_orf_3p | 10549 |
| B3M10000038D10 | 779 | EFA103504 | 5028 | EFA1c0033_orf_94p | 10671 |
| E3M10000038D11 | 780 | EFA103571 | 5030 | EFA1c0044_orf_101p | 10879 |
| E3M10000038D12 | 781 | EFA101540 | 4947 | EFA1c0012_orf_4p | 10487 |
| E3M10000038E02 | 782 | EFA100704 | 4887 | EFA1c0010_orf_4p | 10482 |
| E3M10000038E03 | 783 | EFA101159 | 4916 | EFA1c0022_orf_2p | 10543 |
| E3M10000038E04 | 784 | EFA101540 | 4947 | EFA1c0012_orf_4p | 10487 |
| E3M10000038E05 | 785 | EFA102656 | 5004 | EFA1c0039_orf_26p | 10734 |
| E3M10000038E07 | 786 | EFA102655 | 5003 | EFA1c0039_orf_25p | 10733 |
| E3M10000038E08 | 787 | EFA101121 | 4912 | EFA1c0036_orf_112p | 10686 |
| E3M10000038E11 | 788 | EFA102780 | 5010 | EFA1c0045_orf_101p | 10908 |
| E3M10000038F02 | 789 | EFA102541 | 4998 | EFA1c0028_orf_3p | 10602 |
| E3M10000038F04 | 790 | EFA101686 | 4953 | EFA1c0045_orf_63p | 10940 |
| E3M10000038F05 | 791 | EFA101160 | 4917 | EFA1c0022_orf_3p | 10549 |
| E3M10000038F05 | 791 | EFA101161 | 4918 | EFA1c0022_orf_4p | 10551 |
| E3M10000038F06 | 792 | EFA103571 | 5030 | EFA1c0044_orf_101p | 10879 |
| E3M10000038F07 | 793 | EFA103210 | 5022 | EFA1c0036_orf_119p | 10688 |
| E3M10000038F09 | 794 | EFA102185 | 4980 | EFA1c0045_orf_95p | 10950 |
| E3M10000038F10 | 795 | EFA101080 | 4909 | #N/A | #N/A |
| E3M10000038F11 | 796 | EFA100740 | 4889 | EFA1c0022_orf_22p | 10536 |
| E3M10000038G02 | 797 | EFA100919 | 4901 | EFA1c0013_orf_12p | 10491 |
| E3M10000038G03 | 798 | EFA101414 | 4939 | EFA1c0022_orf_15p | 10528 |
| E3M10000038G06 | 799 | EFA100704 | 4887 | EFA1c0010_orf_4p | 10482 |
| E3M10000038G07 | 800 | EFA102352 | 4990 | EFA1c0032_orf_21p | 10635 |
| E3M10000038G07 | 800 | EFA102353 | 4991 | EFA1c0032_orf_22p | 10636 |
| E3M10000038G11 | 801 | EFA102541 | 4998 | EFA1c0028_orf_3p | 10602 |
| E3M10000038H02 | 802 | EFA101414 | 4939 | EFA1c0022_orf_15p | 10528 |
| E3M10000038H05 | 803 | EFA101160 | 4917 | EFA1c0022_orf_3p | 10549 |
| E3M10000038H06 | 804 | EFA100295 | 4873 | EFA1c0021_orf_15p | 10517 |
| E3M10000038H07 | 805 | EFA101417 | 4942 | EFA1c0022_orf_18p | 10531 |
| E3M10000038H08 | 806 | EFA100295 | 4873 | EFA1c0021_orf_15p | 10517 |
| E3M10000038H09 | 807 | EFA102802 | 5012 | EFA1c0043_orf_18p | 10854 |
| E3M10000038H10 | 808 | EFA101541 | 4948 | EFA1c0012_orf_5p | 10488 |
| E3M10000039A02 | 809 | EFA101736 | 4955 | EFA1c0041_orf_14p | 10775 |
| E3M10000039A02 | 809 | EFA101737 | 4956 | EFA1c0041_orf_15p | 10778 |
| E3M10000039A06 | 810 | EFA102656 | 5004 | EFA1c0039_orf_26p | 10734 |
| E3M10000039A07 | 811 | EFA102006 | 4973 | EFA1c0025_orf_17p | 10580 |
| E3M10000039A08 | 812 | EFA101162 | 4919 | EFA1c0022_orf_5p | 10555 |
| E3M10000039A10 | 813 | EFA101257 | 4925 | EFA1c0045_orf_159p | 10917 |
| E3M10000039A11 | 814 | EFA101412 | 4937 | EFA1c0022_orf_14p | 10527 |
| E3M10000039B01 | 815 | EFA102541 | 4998 | EFA1c0028_orf_3p | 10602 |
| E3M10000039B03 | 816 | EFA101160 | 4917 | EFA1c0022_orf_3p | 10549 |
| E3M10000039B04 | 817 | EFA101415 | 4940 | EFA1c0022_orf_16p | 10529 |
| E3M10000039B04 | 817 | EFA101416 | 4941 | EFA1c0022_orf_17p | 10530 |
| E3M10000039B06 | 818 | EFA100870 | 4899 | EFA1c0031_orf_36p | 10627 |
| E3M10000039B07 | 819 | EFA102110 | 4978 | EFA1c0042_orf_99p | 10841 |
| E3M10000039B08 | 820 | EFA101416 | 4941 | EFA1c0022_orf_17p | 10530 |
| E3M10000039B09 | 821 | EFA101792 | 4961 | EFA1c0042_orf_113p | 10805 |
| E3M10000039B11 | 822 | EFA101080 | 4909 | #N/A | #N/A |
| E3M10000039C02 | 823 | EFA103062 | 5019 | EFA1c0030_orf_19p | 10615 |
| E3M10000039C04 | 824 | EFA101162 | 4919 | EFA1c0022_orf_5p | 10555 |
| E3M10000039C05 | 825 | EFA100739 | 4888 | EFA1c0022_orf_23p | 10537 |
| E3M10000039C06 | 826 | EFA103504 | 5028 | EFA1c0033_orf_94p | 10671 |
| E3M10000039C07 | 827 | EFA101791 | 4960 | EFA1c0042_orf_112p | 10804 |
| E3M10000039C07 | 827 | EFA101792 | 4961 | EFA1c0042_orf_113p | 10805 |
| E3M10000039C08 | 828 | EFA101159 | 4916 | EFA1c0022_orf_2p | 10543 |
| E3M10000039C09 | 829 | EFA102503 | 4996 | EFA1c0032_orf_32p | 10643 |
| E3M10000039C10 | 830 | EFA101162 | 4919 | EFA1c0022_orf_5p | 10555 |
| E3M10000039D02 | 831 | EFA101165 | 4922 | EFA1c0022_orf_8p | 10559 |
| E3M10000039D03 | 832 | EFA102655 | 5003 | EFA1c0039_orf_25p | 10733 |
| E3M10000039D04 | 833 | EFA102656 | 5004 | EFA1c0039_orf_26p | 10734 |
| E3M10000039D06 | 834 | EFA101540 | 4947 | EFA1c0012_orf_4p | 10487 |
| E3M10000039E01 | 835 | EFA102201 | 4982 | #N/A | #N/A |
| E3M10000039E02 | 836 | EFA101540 | 4947 | EFA1c0012_orf_4p | 10487 |
| E3M10000039E03 | 837 | EFA100919 | 4901 | EFA1c0013_orf_12p | 10491 |
| E3M10000039E05 | 838 | EFA101686 | 4953 | EFA1c0045_orf_63p | 10940 |
| E3M10000039E07 | 839 | EFA103295 | 5024 | EFA1c0032_orf_1p | 10633 |
| E3M10000039E08 | 840 | EFA101685 | 4952 | EFA1c0041_orf_55p | 10791 |
| E3M10000039F01 | 841 | EFA102656 | 5004 | EFA1c0039_orf_26p | 10734 |
| E3M10000039F02 | 842 | EFA103021 | 5015 | EFA1c0030_orf_16p | 10612 |
| E3M10000039F03 | 843 | EFA102788 | 5011 | EFA1c0033_orf_41p | 10661 |
| E3M10000039F03 | 843 | EFA103375 | 5027 | EFA1c0033_orf_40p | 10660 |
| E3M10000039F06 | 844 | EFA100739 | 4888 | EFA1c0022_orf_23p | 10537 |
| E3M10000039F07 | 845 | EFA102541 | 4998 | EFA1c0028_orf_3p | 10602 |
| E3M10000039F08 | 846 | EFA101162 | 4919 | EFA1c0022_orf_5p | 10555 |
| E3M10000039G01 | 847 | EFA102502 | 4995 | EFA1c0031_orf_36p | 10627 |
| E3M10000039G02 | 848 | EFA101686 | 4953 | EFA1c0045_orf_63p | 10940 |
| E3M10000039G05 | 849 | EFA100919 | 4901 | EFA1c0013_orf_12p | 10491 |
| E3M10000039G07 | 850 | EFA101686 | 4953 | EFA1c0045_orf_63p | 10940 |
| E3M10000039G09 | 851 | EFA102541 | 4998 | EFA1c0028_orf_3p | 10602 |
| E3M10000039G10 | 852 | EFA101682 | 4951 | EFA1c0041_orf_53p | 10789 |
| E3M10000039H02 | 853 | EFA101160 | 4917 | EFA1c0022_orf_3p | 10549 |
| E3M10000039H07 | 854 | EFA101080 | 4909 | #N/A | #N/A |
| E3M10000039H08 | 855 | EFA101121 | 4912 | EFA1c0036_orf_112p | 10686 |
| E3M10000039H10 | 856 | EFA101413 | 4938 | #N/A | #N/A |
| E3M10000039H11 | 857 | EFA101120 | 4911 | EFA1c0036_orf_113p | 10687 |
| E3M10000039H11 | 857 | EFA101121 | 4912 | EFA1c0036_orf_112p | 10686 |
| E3M10000040A03 | 858 | EFA101123 | 4913 | EFA1c0040_orf_22p | 10748 |
| E3M10000040A05 | 859 | EFA101080 | 4909 | #N/A | #N/A |
| E3M10000040A07 | 860 | EFA100157 | 4865 | EFA1c0034_orf_63p | 10673 |
| E3M10000040A09 | 861 | EFA102502 | 4995 | EFA1c0031_orf_36p | 10627 |
| E3M10000040A10 | 862 | EFA101417 | 4942 | EFA1c0022_orf_18p | 10531 |
| E3M10000040A11 | 863 | EFA101685 | 4952 | EFA1c0041_orf_55p | 10791 |
| E3M10000040B01 | 864 | EFA102788 | 5011 | EFA1c0033_orf_41p | 10661 |
| E3M10000040B02 | 865 | EFA102655 | 5003 | EFA1c0039_orf_25p | 10733 |
| E3M10000040B05 | 866 | EFA100190 | 4867 | EFA1c0010_orf_2p | 10480 |
| E3M10000040B05 | 866 | EFA103268 | 5023 | EFA1c0010_orf_1p | 10479 |
| E3M10000040B06 | 867 | EFA102518 | 4997 | EFA1c0032_orf_46p | 10647 |
| E3M10000040B08 | 868 | EFA100919 | 4901 | EFA1c0013_orf_12p | 10491 |
| E3M10000040B09 | 869 | EFA102502 | 4995 | EFA1c0031_orf_36p | 10627 |
| E3M10000040B10 | 870 | EFA102656 | 5004 | EFA1c0039_orf_26p | 10734 |
| E3M10000040B11 | 871 | EFA102764 | 5008 | EFA1c0008_orf_3p | 10478 |
| E3M10000040B12 | 872 | EFA100210 | 4870 | EFA1c0022_orf_9p | 10560 |
| E3M10000040C02 | 873 | EFA101080 | 4909 | #N/A | #N/A |
| E3M10000040C05 | 874 | EFA102501 | 4994 | EFA1c0031_orf_35p | 10626 |
| E3M10000040C06 | 875 | EFA102091 | 4977 | EFA1c0010_orf_3p | 10481 |
| E3M10000040C07 | 876 | EFA101121 | 4912 | EFA1c0036_orf_112p | 10686 |
| E3M10000040C08 | 877 | EFA102780 | 5010 | EFA1c0045_orf_101p | 10908 |
| E3M10000040C09 | 878 | EFA100165 | 4866 | EFA1c0032_orf_23p | 10637 |
| E3M10000040C09 | 878 | EFA102353 | 4991 | EFA1c0032_orf_22p | 10636 |
| E3M10000040C10 | 879 | EFA101686 | 4953 | EFA1c0045_orf_63p | 10940 |
| B3M10000040C11 | 880 | EFA102501 | 4994 | EFA1c0031_orf_35p | 10626 |
| E3M10000040C12 | 881 | EFA102780 | 5010 | EFA1c0045_orf_101p | 10908 |
| E3M10000040D03 | 882 | EFA102201 | 4982 | #N/A | #N/A |
| E3M10000040D04 | 883 | EFA101080 | 4909 | #N/A | #N/A |
| E3M10000040D08 | 884 | EFA101686 | 4953 | EFA1c0045_orf_63p | 10940 |
| E3M10000040D12 | 885 | EFA101686 | 4953 | EFA1c0045_orf_63p | 10940 |
| E3M10000040E02 | 886 | EFA102051 | 4976 | #N/A | #N/A |
| E3M10000040E10 | 887 | EFA101415 | 4940 | EFA1c0022_orf_16p | 10529 |
| E3M10000040E11 | 888 | EFA103039 | 5018 | EFA1c0043_orf_16p | 10850 |
| E3M10000040E12 | 889 | EFA102091 | 4977 | EFA1c0010_orf_3p | 10481 |
| E3M10000040F01 | 890 | EFA100295 | 4873 | EFA1c0021_orf_15p | 10517 |
| E3M10000040F03 | 891 | EFA102503 | 4996 | EFA1c0032_orf_32p | 10643 |
| E3M10000040F08 | 892 | EFA101080 | 4909 | #N/A | #N/A |
| E3M10000040F09 | 893 | EFA100919 | 4901 | EFA1c0013_orf_12p | 10491 |
| E3M10000040F10 | 894 | EFA102051 | 4976 | #N/A | #N/A |
| E3M10000040G01 | 895 | EFA101415 | 4940 | EFA1c0022_orf_16p | 10529 |
| E3M10000040G02 | 896 | EFA101424 | 4943 | EFA1c0041_orf_39p | 10784 |
| E3M10000040G02 | 896 | EFA101425 | 4944 | EFA1c0041_orf_40p | 10785 |
| E3M10000040G04 | 897 | EFA101141 | 4914 | EFA1c0030_orf_18p | 10614 |
| E3M10000040G05 | 898 | EFA101159 | 4916 | EFA1c0022_orf_2p | 10543 |
| E3M10000040G07 | 899 | EFA101079 | 4908 | #N/A | #N/A |
| E3M10000040G07 | 899 | EFA101080 | 4909 | #N/A | #N/A |
| E3M10000040G08 | 900 | EFA102186 | 4981 | EFA1c0045_orf_94p | 10949 |
| E3M10000040G09 | 901 | EFA103021 | 5015 | EFA1c0030_orf_16p | 10612 |
| E3M10000040G11 | 902 | EFA101414 | 4939 | EFA1c0022_orf_15p | 10528 |
| E3M10000040H02 | 903 | EFA102780 | 5010 | EFA1c0045_orf_101p | 10908 |
| E3M10000040H03 | 904 | EFA100394 | 4876 | EFA1c0034_orf_6p | 10675 |
| E3M10000040H04 | 905 | EFA100642 | 4884 | EFA1c0041_orf_56p | 10792 |
| E3M10000040H04 | 905 | EFA101685 | 4952 | EFA1c0041_orf_55p | 10791 |
| E3M10000040H05 | 906 | EFA100642 | 4884 | EFA1c0041_orf_56p | 10792 |
| E3M10000040H05 | 906 | EFA101685 | 4952 | EFA1c0041_orf_55p | 10791 |
| E3M10000040H09 | 907 | EFA101416 | 4941 | EFA1c0022_orf_17p | 10530 |
| E3M10000040H09 | 907 | EFA101417 | 4942 | EFA1c0022_orf_18p | 10531 |
| E3M10000041A03 | 908 | EFA100615 | 4881 | EFA1c0016_orf_29p | 10501 |
| E3M10000041A05 | 909 | EFA102502 | 4995 | EFA1c0031_orf_36p | 10627 |
| E3M10000041A08 | 910 | EFA100704 | 4887 | EFA1c0010_orf_4p | 10482 |
| E3M10000041A09 | 911 | EFA101354 | 4930 | EFA1c0032_orf_69p | 10648 |
| E3M10000041A10 | 912 | EFA10G001 | 4861 | EFA1c0030_orf_3p | 10618 |
| E3M10000041A11 | 913 | EFA100642 | 4884 | EFA1c0041_orf_56p | 10792 |
| E3M10000041A11 | 913 | EFA101685 | 4952 | EFA1c0041_orf_55p | 10791 |
| E3M10000041B02 | 914 | EFA102656 | 5004 | EFA1c0039_orf_26p | 10734 |
| E3M10000041B03 | 915 | EFA101414 | 4939 | EFA1c0022_orf_15p | 10528 |
| E3M10000041B05 | 916 | EFA102091 | 4977 | EFA1c0010_orf_3p | 10481 |
| E3M10000041B06 | 917 | EFA102091 | 4977 | EFA1c0010_orf_3p | 10481 |
| E3M10000041B08 | 918 | EFA102655 | 5003 | EFA1c0039_orf_25p | 10733 |
| E3M10000041B09 | 919 | EFA101924 | 4970 | EFA1c0044_orf_18p | 10891 |
| E3M10000041B09 | 919 | EFA101925 | 4971 | EFA1c0044_orf_19p | 10893 |
| E3M10000041B10 | 920 | EFA101080 | 4909 | #N/A | #N/A |
| E3M10000041B11 | 921 | EFA101416 | 4941 | EFA1c0022_orf_17p | 10530 |
| E3M10000041B11 | 921 | EFA101417 | 4942 | EFA1c0022_orf_18p | 10531 |
| E3M10000041B12 | 922 | EFA101411 | 4936 | EFA1c0022_orf_13p | 10526 |
| E3M10000041C01 | 923 | EFA100151 | 4864 | EFA1c0021_orf_14p | 10516 |
| E3M10000041C07 | 924 | EFA100739 | 4888 | EFA1c0022_orf_23p | 10537 |
| E3M10000041C08 | 925 | EFA101121 | 4912 | EFA1c0036_orf_112p | 10686 |
| E3M10000041C09 | 926 | EFA103365 | 5026 | EFA1c0022_orf_1p | 10533 |
| E3M10000041C10 | 927 | EFA102503 | 4996 | EFA1c0032_orf_32p | 10643 |
| E3M10000041C11 | 928 | EFA102655 | 5003 | EFA1c0039_orf_25p | 10733 |
| E3M10000041C12 | 929 | EFA100798 | 4897 | EFA1c0042_orf_160p | 10818 |
| E3M10000041D02 | 930 | EFA102502 | 4995 | EFA1c0031_orf_36p | 10627 |
| E3M10000041D03 | 931 | EFA101060 | 4907 | EFA1c0038_orf_73p | 10722 |
| E3M10000041D04 | 932 | EFA100642 | 4884 | EFA1c0041_orf_56p | 10792 |
| E3M10000041D04 | 932 | EFA101685 | 4952 | EFA1c0041_orf_55p | 10791 |
| E3M10000041D05 | 933 | EFA101080 | 4909 | #N/A | #N/A |
| E3M10000041D06 | 934 | EFA102656 | 5004 | EFA1c0039_orf_26p | 10734 |
| E3M10000041D08 | 935 | EFA101417 | 4942 | EFA1c0022_orf_18p | 10531 |
| E3M10000041D09 | 936 | EFA101120 | 4911 | EFA1c0036_orf_113p | 10687 |
| E3M10000041D10 | 937 | EFA102780 | 5010 | EFA1c0045_orf_101p | 10908 |
| E3M10000041D11 | 938 | EFA100704 | 4887 | EFA1c0010_orf_4p | 10482 |
| E3M10000041D12 | 939 | EFA100394 | 4876 | EFA1c0034_orf_6p | 10675 |
| E3M10000041E02 | 940 | EFA101797 | 4963 | EFA1c0045_orf_167p | 10924 |
| E3M10000041E03 | 941 | EFA102091 | 4977 | EFA1c0010_orf_3p | 10481 |
| E3M10000041E05 | 942 | EFA101415 | 4940 | EFA1c0022_orf_16p | 10529 |
| E3M10000041E07 | 943 | EFA102091 | 4977 | EFA1c0010_orf_3p | 10481 |
| E3M10000041E10 | 944 | EFA100704 | 4887 | EFA1c0010_orf_4p | 10482 |
| E3M10000041E11 | 945 | EFA100190 | 4867 | EFA1c0010_orf_2p | 10480 |
| E3M10000041F03 | 946 | EFA102503 | 4996 | EFA1c0032_orf_32p | 10643 |
| E3M10000041F05 | 947 | EFA102006 | 4973 | EFA1c0025_orf_17p | 10580 |
| E3M10000041F06 | 948 | EFA102501 | 4994 | EFA1c0031_orf_35p | 10626 |
| E3M10000041F07 | 949 | EFA101159 | 4916 | EFA1c0022_orf_2p | 10543 |
| E3M10000041F08 | 950 | EFA100295 | 4873 | EFA1c0021_orf_15p | 10517 |
| E3M10000041F09 | 951 | EFA101417 | 4942 | EFA1c0022_orf_18p | 10531 |
| E3M10000041F10 | 952 | EFA101079 | 4908 | #N/A | #N/A |
| E3M10000041F10 | 952 | EFA101080 | 4909 | #N/A | #N/A |
| E3M10000041F11 | 953 | EFA101160 | 4917 | EFA1c0022_orf_3p | 10549 |
| E3M10000041G02 | 954 | EFA101141 | 4914 | EFA1c0030_orf_18p | 10614 |
| E3M10000041G03 | 955 | EFA102253 | 4984 | EFA1c0038_orf_85p | 10727 |
| E3M10000041G04 | 956 | EFA101685 | 4952 | EFA1c0041_orf_55p | 10791 |
| E3M10000041G06 | 957 | EFA100978 | 4904 | EFA1c0022_orf_27p | 10541 |
| E3M10000041G07 | 958 | EFA101141 | 4914 | EFA1c0030_orf_18p | 10614 |
| E3M10000041G08 | 959 | EFA100704 | 4887 | EFA1c0010_orf_4p | 10482 |
| E3M10000041G09 | 960 | EFA100704 | 4887 | EFA1c0010_orf_4p | 10482 |
| E3M10000041G10 | 961 | EFA100394 | 4876 | EFA1c0034_orf_6p | 10675 |
| E3M10000041G12 | 962 | EFA100394 | 4876 | EFA1c0034_orf_6p | 10675 |
| E3M10000041H04 | 963 | EFA102351 | 4989 | EFA1c0032_orf_20p | 10634 |
| E3M10000041H05 | 964 | EFA100329 | 4875 | EFA1c0041_orf_35p | 10782 |
| E3M10000041H06 | 965 | EFA102502 | 4995 | EFA1c0031_orf_36p | 10627 |
| E3M10000041H07 | 966 | EFA103062 | 5019 | EFA1c0030_orf_19p | 10615 |
| E3M10000041H08 | 967 | EFA101686 | 4953 | EFA1c0045_orf_63p | 10940 |
| E3M10000041H09 | 968 | EFA102788 | 5011 | EFA1c0033_orf_41p | 10661 |
| E3M10000041H10 | 969 | EFA101685 | 4952 | EFA1c0041_orf_55p | 10791 |
| E3M10000041H11 | 970 | EFA102253 | 4984 | EFA1c0038_orf_85p | 10727 |
| E3M10000042A03 | 971 | EFA101120 | 4911 | EFA1c0036_orf_113p | 10687 |
| E3M10000042A03 | 971 | EFA101121 | 4912 | EFA1c0036_orf_112p | 10686 |
| E3M10000042A08 | 972 | EFA102351 | 4989 | EFA1c0032_orf_20p | 10634 |
| E3M10000042A10 | 973 | EFA101121 | 4912 | EFA1c0036_orf_112p | 10686 |
| E3M10000042B01 | 974 | EFA101404 | 4933 | EFA1c0033_orf_55p | 10663 |
| E3M10000042B02 | 975 | EFA100668 | 4885 | EFA1c0035_orf_58p | 10679 |
| E3M10000042B04 | 976 | EFA102186 | 4981 | EFA1c0045_orf_94p | 10949 |
| E3M10000042B04 | 976 | EFA102453 | 4993 | EFA1c0045_orf_203p | 10931 |
| E3M10000042B08 | 977 | EFA101417 | 4942 | EFA1c0022_orf_18p | 10531 |
| E3M10000042B09 | 978 | EFA101797 | 4963 | EFA1c0045_orf_167p | 10924 |
| E3M10000042B10 | 979 | EFA101121 | 4912 | EFA1c0036_orf_112p | 10686 |
| E3M10000042B11 | 980 | EFA101165 | 4922 | EFA1c0022_orf_8p | 10559 |
| E3M10000042C02 | 981 | EFA101150 | 4915 | EFA1c0038_orf_57p | 10719 |
| E3M10000042C03 | 982 | EFA102780 | 5010 | EFA1c0045_orf_101p | 10908 |
| E3M10000042C04 | 983 | EFA102656 | 5004 | EFA1c0039_orf_26p | 10734 |
| E3M10000042C10 | 984 | EFA100151 | 4864 | EFA1c0021_orf_14p | 10516 |
| E3M10000042C10 | 984 | EFA100295 | 4873 | EFA1c0021_orf_15p | 10517 |
| E3M10000042D01 | 985 | EFA100615 | 4881 | EFA1c0016_orf_29p | 10501 |
| E3M10000042D02 | 986 | EFA102501 | 4994 | EFA1c0031_orf_35p | 10626 |
| E3M10000042D03 | 987 | EFA100394 | 4876 | EFA1c0034_orf_6p | 10675 |
| E3M10000042D06 | 988 | EFA102091 | 4977 | EFA1c0010_orf_3p | 10481 |
| E3M10000042D09 | 989 | EFA101141 | 4914 | EFA1c0030_orf_18p | 10614 |
| E3M10000042D11 | 990 | EFA101412 | 4937 | EFA1c0022_orf_14p | 10527 |
| E3M10000042D12 | 991 | EFA100795 | 4896 | EFA1c0043_orf_229p | 10863 |
| E3M10000042E05 | 992 | EFA102501 | 4994 | EFA1c0031_orf_35p | 10626 |
| E3M10000042E12 | 993 | EFA102351 | 4989 | EFA1c0032_orf_20p | 10634 |
| E3M10000042F11 | 994 | EFA101792 | 4961 | EFA1c0042_orf_113p | 10805 |
| E3M10000042G01 | 995 | EFA101412 | 4937 | EFA1c0022_orf_14p | 10527 |
| E3M10000042G05 | 996 | EFA101685 | 4952 | EFA1c0041_orf_55p | 10791 |
| E3M10000042G07 | 997 | EFA101169 | 4923 | EFA1c0024_orf_38p | 10574 |
| E3M10000042G08 | 998 | EFA102780 | 5010 | EFA1c0045_orf_101p | 10908 |
| E3M10000042G11 | 999 | EFA101120 | 4911 | EFA1c0036_orf_113p | 10687 |
| E3M10000042G11 | 999 | EFA101121 | 4912 | EFA1c0036_orf_112p | 10686 |
| E3M10000042G12 | 1000 | EFA102501 | 4994 | EFA1c0031_orf_35p | 10626 |
| E3M10000042H06 | 1001 | EFA101799 | 4964 | EFA1c0045_orf_169p | 10926 |
| E3M10000042H08 | 1002 | EFA101120 | 4911 | EFA1c0036_orf_113p | 10687 |
| E3M10000042H11 | 1003 | EFA100668 | 4885 | EFA1c0035_orf_58p | 10679 |
| E3M10000043A02 | 1004 | EFA101799 | 4964 | EFA1c0045_orf_169p | 10926 |
| E3M10000043A03 | 1005 | EFA101414 | 4939 | EFA1c0022_orf_15p | 10528 |
| E3M10000043A05 | 1006 | EFA102502 | 4995 | EFA1c0031_orf_36p | 10627 |
| E3M10000043A08 | 1007 | EFA100689 | 4886 | EFA1c0038_orf_54p | 10717 |
| E3M10000043A09 | 1008 | EFA101414 | 4939 | EFA1c0022_orf_15p | 10528 |
| E3M10000043A09 | 1008 | EFA101415 | 4940 | EFA1c0022_orf_16p | 10529 |
| E3M10000043A10 | 1009 | EFA101080 | 4909 | #N/A | #N/A |
| E3M10000043A11 | 1010 | EFA102006 | 4973 | EFA1c0025_orf_17p | 10580 |
| E3M10000043B01 | 1011 | EFA100151 | 4864 | EFA1c0021_orf_14p | 10516 |
| E3M10000043B02 | 1012 | EFA101121 | 4912 | EFA1c0036_orf_112p | 10686 |
| E3M10000043B03 | 1013 | EFA103038 | 5017 | EFA1c0030_orf_17p | 10613 |
| E3M10000043B06 | 1014 | EFA101404 | 4933 | EFA1c0033_orf_55p | 10663 |
| E3M10000043B08 | 1015 | EFA101123 | 4913 | EFA1c0040_orf_22p | 10748 |
| E3M10000043B09 | 1016 | EFA101892 | 4969 | EFA1c0017_orf_21p | 10506 |
| E3M10000043B10 | 1017 | EFA102656 | 5004 | EFA1c0039_orf_26p | 10734 |
| E3M10000043B11 | 1018 | EFA100704 | 4887 | EFA1c0010_orf_4p | 10482 |
| E3M10000043B12 | 1019 | EFA100151 | 4864 | EFA1c0021_orf_14p | 10516 |
| E3M10000043C01 | 1020 | EFA102656 | 5004 | EFA1c0039_orf_26p | 10734 |
| E3M10000043C08 | 1021 | EFA101412 | 4937 | EFA1c0022_orf_14p | 10527 |
| E3M10000043C09 | 1022 | EFA100151 | 4864 | EFA1c0021_orf_14p | 10516 |
| E3M10000043D01 | 1023 | EFA101417 | 4942 | EFA1c0022_orf_18p | 10531 |
| E3M10000043D02 | 1024 | EFA102502 | 4995 | EFA1c0031_orf_36p | 10627 |
| E3M10000043D09 | 1025 | EFA102351 | 4989 | EFA1c0032_orf_20p | 10634 |
| E3M10000043D10 | 1026 | EFA101872 | 4967 | EFA1c0042_orf_152p | 10815 |
| E3M10000043D10 | 1026 | EFA101873 | 4968 | EFA1c0042_orf_153p | 10816 |
| E3M10000043D12 | 1027 | EFA102502 | 4995 | EFA1c0031_orf_36p | 10627 |
| E3M10000043E03 | 1028 | EFA100397 | 4877 | EFA1c0041_orf_148p | 10773 |
| E3M10000043E07 | 1029 | EFA101339 | 4928 | EFA1c0040_orf_13p | 10743 |
| E3M10000043E08 | 1030 | EFA101872 | 4967 | EFA1c0042_orf_152p | 10815 |
| E3M10000043E08 | 1030 | EFA101873 | 4968 | EFA1c0042_orf_153p | 10816 |
| E3M10000043E10 | 1031 | EFA102656 | 5004 | EFA1c0039_orf_26p | 10734 |
| E3M10000043E11 | 1032 | EFA102813 | 5013 | EFA1c0043_orf_9p | 10878 |
| E3M10000043F03 | 1033 | EFA102655 | 5003 | EFA1c0039_orf_25p | 10733 |
| E3M10000043F04 | 1034 | EFA102006 | 4973 | EFA1c0025_orf_17p | 10580 |
| E3M10000043F06 | 1035 | EFA100615 | 4881 | EFA1c0016_orf_29p | 10501 |
| E3M10000043F08 | 1036 | EFA101121 | 4912 | EFA1c0036_orf_112p | 10686 |
| E3M10000043F10 | 1037 | EFA101159 | 4916 | EFA1c0022_orf_2p | 10543 |
| E3M10000043F12 | 1038 | EFA101080 | 4909 | #N/A | #N/A |
| E3M10000043G03 | 1039 | EFA102502 | 4995 | EFA1c0031_orf_36p | 10627 |
| E3M10000043G04 | 1040 | EFA102502 | 4995 | EFA1c0031_orf_36p | 10627 |
| E3M10000043G05 | 1041 | EFA101686 | 4953 | EFA1c0045_orf_63p | 10940 |
| E3M10000043G07 | 1042 | EFA100157 | 4865 | EFA1c0034_orf_63p | 10673 |
| E3M10000043G08 | 1043 | EFA101080 | 4909 | #N/A | #N/A |
| E3M10000043G10 | 1044 | EFA101792 | 4961 | EFA1c0042_orf_113p | 10805 |
| E3M10000043G11 | 1045 | EFA101080 | 4909 | #N/A | #N/A |
| E3M10000043G12 | 1046 | EFA101121 | 4912 | EFA1c0036_orf_112p | 10686 |
| E3M10000043H02 | 1047 | EFA101414 | 4939 | EFA1c0022_orf_15p | 10528 |
| E3M10000043H05 | 1048 | EFA101080 | 4909 | #N/A | #N/A |
| E3M10000043H08 | 1049 | EFA100615 | 4881 | EFA1c0016_orf_29p | 10501 |
| E3M10000043H09 | 1050 | EFA102006 | 4973 | EFA1c0025_orf_17p | 10580 |
| E3M10000043H11 | 1051 | EFA102655 | 5003 | EFA1c0039_orf_25p | 10733 |
| E3M10000044C02 | 1052 | EFA100955 | 4902 | EFA1c0022_orf_28p | 10542 |
| E3M10000044E01 | 1053 | EFA102091 | 4977 | EFA1c0010_orf_3p | 10481 |
| K1M10000002F02 | 1054 | KPN101750 | 5037 | KPN1c1723_orf_1p | 11652 |
| K1M10000003C01 | 1055 | KPN103882 | 5040 | KPN1c2848_orf_1p | 11716 |
| K1M10000007F01 | 1057 | KPN104183 | 5041 | KPN1c1646_orf_2p | 11650 |
| K1M10000007F01 | 1057 | KPN106659 | 5049 | KPN1c1646_orf_1p | 11649 |
| K1M10000008C02 | 1058 | KPN107626 | 5051 | #N/A | #N/A |
| K1M10000008C10 | 1059 | KPN101729 | 5036 | KPN1c1566_orf_1p | 11647 |
| K1M10000008G10 | 1060 | KPN106840 | 5050 | KPN1c2087_orf_1p | 11664 |
| K1M10000009D04 | 1061 | KPN107776 | 5052 | KPN1c4041_orf_1p | 11771 |
| K1M10000013E04 | 1062 | KPN105779 | 5047 | KPN1c4012_orf_1p | 11770 |
| K1M10000020B02 | 1065 | KPN101729 | 5036 | KPN1c1566_orf_1p | 11647 |
| K1M10000022C10 | 1067 | KPN100854 | 5033 | KPN1c0845_orf_1p | 11630 |
| K1M10000030C07 | 1070 | KPN104716 | 5045 | KPN1c3094_orf_5p | 11757 |
| K1M10000030E07 | 1071 | KPN104538 | 5044 | KPN1c2918_orf_2p | 11726 |
| K1M10000032E11 | 1073 | KPN101729 | 5036 | KPN1c1566_orf_1p | 11647 |
| K1M10000033B02 | 1074 | KPN101729 | 5036 | KPN1c1566_orf_1p | 11647 |
| K1M10000033E01 | 1075 | KPN100432 | 5032 | KPN1c0331_orf_1p | 11628 |
| K1M10000036G08 | 1076 | KPN106044 | 5048 | #N/A | #N/A |
| K1M10000037D10 | 1077 | KPN104281 | 5042 | KPN1c3000_orf_3p | 11742 |
| K1M10000038H09 | 1078 | KPN102057 | 5038 | KPN1c1958_orf_1p | 11661 |
| K1M10000039H03 | 1079 | KPN106840 | 5050 | KPN1c2087_orf_1p | 11664 |
| K1M10000043D05 | 1081 | KPN102638 | 5039 | KPN1c2127_orf_1p | 11667 |
| K1M10000043H10 | 1082 | KPN105722 | 5046 | #N/A | #N/A |
| K1M10000044D08 | 1084 | KPN104430 | 5043 | #N/A | #N/A |
| K1M10000044G05 | 1086 | KPN101026 | 5035 | KPN1c0875_orf_1p | 11631 |
| K1M10000045A07 | 1087 | KPN101022 | 5034 | KPN1c1316_orf_3p | 11642 |
| K1M10000045D10 | 1088 | KPN102638 | 5039 | KPN1c2127_orf_1p | 11667 |
| P1M10000008C06 | 1092 | PA2424 | 5107 | #N/A | #N/A |
| P1M10000008G04 | 1093 | PA0337 | 5060 | #N/A | #N/A |
| P1M10000010C03 | 1094 | PA4997 | 5202 | #N/A | #N/A |
| P1M10000014H10 | 1095 | PA4252 | 5168 | #N/A | #N/A |
| P1M10000014H10 | 1095 | PA4253 | 5169 | #N/A | #N/A |
| P1M10000015C06 | 1096 | PA0413 | 5064 | #N/A | #N/A |
| P1M10000015C06 | 1096 | PA0414 | 5065 | #N/A | #N/A |
| P1M10000015C09 | 1097 | PA3041 | 5124 | #N/A | #N/A |
| P1M10000016C04 | 1098 | PA2680 | 5117 | #N/A | #N/A |
| P1M10000018B01 | 1099 | PA4264 | 5177 | #N/A | #N/A |
| P1M10000018C01 | 1100 | PA4264 | 5177 | #N/A | #N/A |
| P1M10000018E01 | 1101 | PA4067 | 5151 | #N/A | #N/A |
| P1M10000018G01 | 1102 | PA4067 | 5151 | #N/A | #N/A |
| P1M10000019F01 | 1103 | PA4271 | 5180 | #N/A | #N/A |
| P1M10000019F01 | 1103 | PA4272 | 5181 | #N/A | #N/A |
| P1M10000021G03 | 1104 | PA4264 | 5177 | #N/A | #N/A |
| P1M10000021G05 | 1105 | PA4251 | 5167 | #N/A | #N/A |
| P1M10000022D09 | 1106 | PA5299 | 5211 | #N/A | #N/A |
| P1M10000024D06 | 1107 | PA3160 | 5130 | #N/A | #N/A |
| P1M10000024E06 | 1108 | PA4888 | 5200 | #N/A | #N/A |
| P1M10000024H03 | 1109 | PA2313 | 5105 | #N/A | #N/A |
| P1M10000025A06 | 1110 | PA2222 | 5104 | #N/A | #N/A |
| P1M10000025G07 | 1111 | PA3153 | 5128 | #N/A | #N/A |
| P1M10000025H07 | 1112 | PA3153 | 5128 | #N/A | #N/A |
| P1M10000025H07 | 1112 | PA3154 | 5129 | #N/A | #N/A |
| P1M10000026E06 | 1113 | PA0715 | 5074 | #N/A | #N/A |
| P1M10000026F04 | 1114 | PA2222 | 5104 | #N/A | #N/A |
| P1M10000026G09 | 1115 | PA3011 | 5122 | #N/A | #N/A |
| P1M10000026H02 | 1116 | PA3013 | 5123 | #N/A | #N/A |
| P1M10000026H05 | 1117 | PA3154 | 5129 | #N/A | #N/A |
| P1M10000027A06 | 1118 | PA4257 | 5172 | #N/A | #N/A |
| P1M10000027B02 | 1119 | PA3154 | 5129 | #N/A | #N/A |
| P1M10000027G05 | 1120 | PA2313 | 5105 | #N/A | #N/A |
| P1M10000028A08 | 1121 | PA0788 | 5075 | #N/A | #N/A |
| P1M10000028B01 | 1122 | PA4263 | 5176 | #N/A | #N/A |
| P1M10000028E02 | 1123 | PA2584 | 5112 | #N/A | #N/A |
| P1M10000029A09 | 1124 | PA3154 | 5129 | #N/A | #N/A |
| P1M10000029G03 | 1125 | PA1301 | 5083 | #N/A | #N/A |
| P1M10000029H05 | 1126 | PA0353 | 5061 | #N/A | #N/A |
| P1M10000032F04 | 1127 | PA0265 | 5058 | #N/A | #N/A |
| P1M10000033A02 | 1128 | PA3068 | 5126 | #N/A | #N/A |
| P1M10000033B08 | 1129 | PA4244 | 5160 | #N/A | #N/A |
| P1M10000033E03 | 1130 | PA3984 | 5147 | #N/A | #N/A |
| P1M10000033F01 | 1131 | PA1986 | 5095 | #N/A | #N/A |
| P1M10000033G08 | 1132 | PA2009 | 5096 | #N/A | #N/A |
| P1M10000035A06 | 1133 | PA4249 | 5165 | #N/A | #N/A |
| P1M10000037B12 | 1134 | PA4254 | 5170 | #N/A | #N/A |
| P1M10000037G12 | 1135 | PA5076 | 5204 | #N/A | #N/A |
| P1M10000038B08 | 1136 | PA4070 | 5152 | #N/A | #N/A |
| P1M10000038C03 | 1137 | PA3931 | 5146 | #N/A | #N/A |
| P1M10000038C06 | 1138 | PA2197 | 5103 | #N/A | #N/A |
| P1M10000038F04 | 1139 | PA5207 | 5208 | #N/A | #N/A |
| P1M10000038G02 | 1140 | PA4542 | 5192 | #N/A | #N/A |
| P1M10000039G05 | 1141 | PA3764 | 5141 | #N/A | #N/A |
| P1M10000039G12 | 1142 | PA5567 | 5220 | #N/A | #N/A |
| P1M10000040C01 | 1143 | PA4105 | 5154 | #N/A | #N/A |
| P1M10000040C04 | 1144 | PA1115 | 5081 | #N/A | #N/A |
| P1M10000040D04 | 1145 | PA0378 | 5062 | #N/A | #N/A |
| P1M10000040D05 | 1146 | PA5209 | 5209 | #N/A | #N/A |
| P1M10000040E10 | 1147 | PA2128 | 5100 | #N/A | #N/A |
| P1M10000040H03 | 1148 | PA1115 | 5081 | #N/A | #N/A |
| P1M10000041A12 | 1149 | PA4254 | 5170 | #N/A | #N/A |
| P1M10000041B02 | 1150 | PA2128 | 5100 | #N/A | #N/A |
| P1M10000041E01 | 1151 | PA2398 | 5106 | #N/A | #N/A |
| P1M10000041F01 | 1152 | PA4681 | 5196 | #N/A | #N/A |
| P1M10000042B12 | 1153 | PA0642 | 5072 | #N/A | #N/A |
| P1M10000042E08 | 1154 | PA4252 | 5168 | #N/A | #N/A |
| P1M10000042E08 | 1154 | PA4253 | 5169 | #N/A | #N/A |
| P1M10000043A03 | 1155 | PA3006 | 5121 | #N/A | #N/A |
| P1M10000043D06 | 1156 | PA3764 | 5141 | #N/A | #N/A |
| P1M10000044F07 | 1157 | PA4244 | 5160 | #N/A | #N/A |
| P1M10000046B03 | 1158 | PA1462 | 5087 | #N/A | #N/A |
| P1M10000046C07 | 1159 | PA2671 | 5116 | #N/A | #N/A |
| P1M10000046C08 | 1160 | PA0472 | 5069 | #N/A | #N/A |
| P1M10000046C09 | 1161 | PA3764 | 5141 | #N/A | #N/A |
| P1M10000046G11 | 1162 | PA1115 | 5081 | #N/A | #N/A |
| P1M10000047B04 | 1163 | PA3006 | 5121 | #N/A | #N/A |
| P1M10000047E11 | 1164 | PA2684 | 5118 | #N/A | #N/A |
| P1M10000047F07 | 1165 | PA4506 | 5190 | #N/A | #N/A |
| P1M10000047G10 | 1166 | PA4259 | 5174 | #N/A | #N/A |
| P1M10000048A03 | 1167 | PA4105 | 5154 | #N/A | #N/A |
| P1M10000049E08 | 1168 | PA4272 | 5181 | #N/A | #N/A |
| P1M10000049G10 | 1169 | PA4027 | 5149 | #N/A | #N/A |
| P1M10000050G11 | 1170 | PA4249 | 5165 | #N/A | #N/A |
| P1M10000051D11 | 1171 | PA1365 | 5085 | #N/A | #N/A |
| P1M10000051F01 | 1172 | PA1115 | 5081 | #N/A | #N/A |
| P1M10000052C03 | 1173 | PA0938 | 5078 | #N/A | #N/A |
| P1M10000052C12 | 1174 | PA5076 | 5204 | #N/A | #N/A |
| P1M10000052E04 | 1175 | PA1398 | 5086 | #N/A | #N/A |
| P1M10000053B12 | 1176 | PA5436 | 5215 | #N/A | #N/A |
| P1M10000053C02 | 1177 | PA0353 | 5061 | #N/A | #N/A |
| P1M10000053E07 | 1178 | PA4254 | 5170 | #N/A | #N/A |
| P1M10000053F08 | 1179 | PA1270 | 5082 | #N/A | #N/A |
| P1M10000055A11 | 1180 | PA5076 | 5204 | #N/A | #N/A |
| P1M10000055C08 | 1181 | PA1493 | 5088 | #N/A | #N/A |
| P1M10000055E05 | 1182 | PA5507 | 5219 | #N/A | #N/A |
| P1M10000056C07 | 1183 | PA1360 | 5084 | #N/A | #N/A |
| P1M10000056F05 | 1184 | PA4258 | 5173 | #N/A | #N/A |
| P1M10000056F05 | 1184 | PA4259 | 5174 | #N/A | #N/A |
| P1M10000056F06 | 1185 | PA2634 | 5114 | #N/A | #N/A |
| P1M10000056G01 | 1186 | PA5076 | 5204 | #N/A | #N/A |
| P1M10000058B07 | 1187 | PA5436 | 5215 | #N/A | #N/A |
| P1M10000059B04 | 1188 | PA4375 | 5186 | #N/A | #N/A |
| P1M10000059B10 | 1189 | PA4269 | 5179 | #N/A | #N/A |
| P1M10000059B11 | 1190 | PA0934 | 5077 | #N/A | #N/A |
| P1M10000059D11 | 1191 | PA4027 | 5149 | #N/A | #N/A |
| P1M10000059H08 | 1192 | PA4027 | 5149 | #N/A | #N/A |
| P1M10000059H09 | 1193 | PA4271 | 5180 | #N/A | #N/A |
| P1M10000060E03 | 1194 | PA0423 | 5067 | #N/A | #N/A |
| P1M10000060H02 | 1195 | PA0221 | 5057 | #N/A | #N/A |
| P1M10000060H04 | 1196 | PA4473 | 5189 | #N/A | #N/A |
| P1M10000061B04 | 1197 | PA2726 | 5119 | #N/A | #N/A |
| P1M10000061E04 | 1198 | PA4244 | 5160 | #N/A | #N/A |
| P1M10000061F04 | 1199 | PA3522 | 5136 | #N/A | #N/A |
| P1M10000062A12 | 1200 | PA4598 | 5194 | #N/A | #N/A |
| P1M10000062C03 | 1201 | PA0321 | 5059 | #N/A | #N/A |
| P1M10000062C04 | 1202 | PA4254 | 5170 | #N/A | #N/A |
| P1M10000062C07 | 1203 | PA4251 | 5167 | #N/A | #N/A |
| P1M10000062C12 | 1204 | PA5316 | 5212 | #N/A | #N/A |
| P1M10000062D07 | 1205 | PA4247 | 5163 | #N/A | #N/A |
| P1M10000062D08 | 1206 | PA0882 | 5076 | #N/A | #N/A |
| P1M10000062E08 | 1207 | PA4248 | 5164 | #N/A | #N/A |
| P1M10000062E08 | 1207 | PA4249 | 5165 | #N/A | #N/A |
| P1M10000062F06 | 1208 | PA0028 | 5053 | #N/A | #N/A |
| P1M10000062G11 | 1209 | PA4506 | 5190 | #N/A | #N/A |
| P1M10000062H01 | 1210 | PA3121 | 5127 | #N/A | #N/A |
| P1M10000062H04 | 1211 | PA4254 | 5170 | #N/A | #N/A |
| P1M10000063F02 | 1212 | PA2684 | 5118 | #N/A | #N/A |
| P1M10000063G02 | 1213 | PA4262 | 5175 | #N/A | #N/A |
| P1M10000063H02 | 1214 | PA4081 | 5153 | #N/A | #N/A |
| P1M10000064A10 | 1215 | PA4268 | 5178 | #N/A | #N/A |
| P1M10000064C02 | 1216 | PA0650 | 5073 | #N/A | #N/A |
| P1M10000064C03 | 1217 | PA5030 | 5203 | #N/A | #N/A |
| P1M10000064D03 | 1218 | PA0129 | 5055 | #N/A | #N/A |
| P1M10000064E05 | 1219 | PA4512 | 5191 | #N/A | #N/A |
| P1M10000064G12 | 1220 | PA2147 | 5101 | #N/A | #N/A |
| P1M10000064H07 | 1221 | PA1072 | 5080 | #N/A | #N/A |
| P1M10000065A04 | 1222 | PA3522 | 5136 | #N/A | #N/A |
| P1M10000065B07 | 1223 | PA4347 | 5184 | #N/A | #N/A |
| P1M10000065C03 | 1224 | PA4347 | 5184 | #N/A | #N/A |
| P1M10000065C05 | 1225 | PA0642 | 5072 | #N/A | #N/A |
| P1M10000065D06 | 1226 | PA4347 | 5184 | #N/A | #N/A |
| P1M10000065F01 | 1227 | PA2494 | 5111 | #N/A | #N/A |
| P1M10000065G06 | 1228 | PA0423 | 5067 | #N/A | #N/A |
| P1M10000065H07 | 1229 | PA1019 | 5079 | #N/A | #N/A |
| P1M10000066A10 | 1230 | PA4709 | 5197 | #N/A | #N/A |
| P1M10000066A11 | 1231 | PA2594 | 5113 | #N/A | #N/A |
| P1M10000066F04 | 1232 | PA4024 | 5148 | #N/A | #N/A |
| P1M10000067A05 | 1233 | PA3876 | 5144 | #N/A | #N/A |
| P1M10000067A05 | 1233 | PA3877 | 5145 | #N/A | #N/A |
| P1M10000067A06 | 1234 | PA0419 | 5066 | #N/A | #N/A |
| P1M10000067A08 | 1235 | PA0600 | 5071 | #N/A | #N/A |
| P1M10000067C04 | 1236 | PA3845 | 5142 | #N/A | #N/A |
| P1M10000067C06 | 1237 | PA4433 | 5188 | #N/A | #N/A |
| P1M10000067D05 | 1238 | PA3479 | 5134 | #N/A | #N/A |
| P1M10000067F05 | 1239 | PA3643 | 5137 | #N/A | #N/A |
| P1M10000067G05 | 1240 | PA5199 | 5207 | #N/A | #N/A |
| P1M10000068A09 | 1241 | PA0353 | 5061 | #N/A | #N/A |
| P1M10000068D04 | 1242 | PA5388 | 5213 | #N/A | #N/A |
| P1M10000068F04 | 1243 | PA4237 | 5158 | #N/A | #N/A |
| P1M10000068F08 | 1244 | PA5193 | 5206 | #N/A | #N/A |
| P1M10000068G01 | 1245 | PA3716 | 5140 | #N/A | #N/A |
| P1M10000068H05 | 1246 | PA4268 | 5178 | #N/A | #N/A |
| P1M10000069D09 | 1247 | PA4246 | 5162 | #N/A | #N/A |
| P1M10000069G06 | 1248 | PA4246 | 5162 | #N/A | #N/A |
| P1M10000069H02 | 1249 | PA4433 | 5188 | #N/A | #N/A |
| P1M10000070A05 | 1250 | PA2470 | 5109 | #N/A | #N/A |
| P1M10000070B10 | 1251 | PA5393 | 5214 | #N/A | #N/A |
| P1M10000070C06 | 1252 | PA4237 | 5158 | #N/A | #N/A |
| P1M10000070D08 | 1253 | PA4105 | 5154 | #N/A | #N/A |
| P1M10000070E03 | 1254 | PA4709 | 5197 | #N/A | #N/A |
| P1M10000070G06 | 1255 | PA3374 | 5133 | #N/A | #N/A |
| P1M10000070G12 | 1256 | PA3121 | 5127 | #N/A | #N/A |
| P1M10000070H06 | 1257 | PA3374 | 5133 | #N/A | #N/A |
| P1M10000071A03 | 1258 | PA4251 | 5167 | #N/A | #N/A |
| P1M10000071C01 | 1259 | PA4251 | 5167 | #N/A | #N/A |
| P1M10000071E04 | 1260 | PA3484 | 5135 | #N/A | #N/A |
| P1M10000071F01 | 1261 | PA0506 | 5070 | #N/A | #N/A |
| P1M10000073A06 | 1262 | PA4246 | 5162 | #N/A | #N/A |
| P1M10000073B10 | 1263 | PA5248 | 5210 | #N/A | #N/A |
| P1M10000073D04 | 1264 | PA1115 | 5081 | #N/A | #N/A |
| P1M10000073D09 | 1265 | PA1918 | 5094 | #N/A | #N/A |
| P1M10000073G03 | 1266 | PA5248 | 5210 | #N/A | #N/A |
| P1M10000074B01 | 1267 | PA4771 | 5199 | #N/A | #N/A |
| P1M10000074B04 | 1268 | PA1684 | 5091 | #N/A | #N/A |
| P1M10000074E04 | 1269 | PA0120 | 5054 | #N/A | #N/A |
| P1M10000074E09 | 1270 | PA3479 | 5134 | #N/A | #N/A |
| P1M10000074F10 | 1271 | PA1019 | 5079 | #N/A | #N/A |
| P1M10000074G12 | 1272 | PA4244 | 5160 | #N/A | #N/A |
| P1M10000074G12 | 1272 | PA4245 | 5161 | #N/A | #N/A |
| P1M10000075A04 | 1273 | PA3279 | 5131 | #N/A | #N/A |
| P1M10000075A04 | 1273 | PA3280 | 5132 | #N/A | #N/A |
| P1M10000075B03 | 1274 | PA4576 | 5193 | #N/A | #N/A |
| P1M10000075F02 | 1275 | PA4254 | 5170 | #N/A | #N/A |
| P1M10000075G05 | 1276 | PA3709 | 5139 | #N/A | #N/A |
| P1M10000076D05 | 1277 | PA1876 | 5093 | #N/A | #N/A |
| P1M10000076D10 | 1278 | PA1636 | 5090 | #N/A | #N/A |
| P1M10000077A08 | 1279 | PA3479 | 5134 | #N/A | #N/A |
| P1M10000077C08 | 1280 | PA1019 | 5079 | #N/A | #N/A |
| P1M10000077E04 | 1281 | PA3522 | 5136 | #N/A | #N/A |
| P1M10000077H05 | 1282 | PA4246 | 5162 | #N/A | #N/A |
| P1M10000079A10 | 1283 | PA4576 | 5193 | #N/A | #N/A |
| P1M10000079B10 | 1284 | PA4576 | 5193 | #N/A | #N/A |
| P1M10000079C10 | 1285 | PA4576 | 5193 | #N/A | #N/A |
| P1M10000079D01 | 1286 | PA1547 | 5089 | #N/A | #N/A |
| P1M10000079D10 | 1287 | PA5490 | 5217 | #N/A | #N/A |
| P1M10000079F06 | 1288 | PA3006 | 5121 | #N/A | #N/A |
| P1M10000080B01 | 1289 | PA3866 | 5143 | #N/A | #N/A |
| P1M10000080B06 | 1290 | PA4244 | 5160 | #N/A | #N/A |
| P1M10000080B06 | 1290 | PA4245 | 5161 | #N/A | #N/A |
| P1M10000080C01 | 1291 | PA0469 | 5068 | #N/A | #N/A |
| P1M10000080C06 | 1292 | PA4250 | 5166 | #N/A | #N/A |
| P1M10000080E04 | 1293 | PA4250 | 5166 | #N/A | #N/A |
| P1M10000081D12 | 1294 | PA3006 | 5121 | #N/A | #N/A |
| P1M10000081G05 | 1295 | PA4037 | 5150 | #N/A | #N/A |
| P1M10000081H05 | 1296 | PA4316 | 5182 | #N/A | #N/A |
| P1M10000082A05 | 1297 | PA0401 | 5063 | #N/A | #N/A |
| P1M10000082B04 | 1298 | PA3006 | 5121 | #N/A | #N/A |
| P1M10000082C05 | 1299 | PA4246 | 5162 | #N/A | #N/A |
| P1M10000082D05 | 1300 | PA4256 | 5171 | #N/A | #N/A |
| P1M10000082E05 | 1301 | PA4246 | 5162 | #N/A | #N/A |
| P1M10000083A11 | 1302 | PA3006 | 5121 | #N/A | #N/A |
| P1M10000083B01 | 1303 | PA4271 | 5180 | #N/A | #N/A |
| P1M10000083B12 | 1304 | PA4268 | 5178 | #N/A | #N/A |
| P1M10000083C11 | 1305 | PA4242 | 5159 | #N/A | #N/A |
| P1M10000083C12 | 1306 | PA3006 | 5121 | #N/A | #N/A |
| P1M10000084A04 | 1307 | PA4942 | 5201 | #N/A | #N/A |
| P1M10000084D03 | 1308 | PA3006 | 5121 | #N/A | #N/A |
| P1M10000084E04 | 1309 | PA5493 | 5218 | #N/A | #N/A |
| P1M10000084B11 | 1310 | PA2196 | 5102 | #N/A | #N/A |
| P1M10000084F08 | 1311 | PA4271 | 5180 | #N/A | #N/A |
| P1M10000085D06 | 1312 | PA3006 | 5121 | #N/A | #N/A |
| P1M10000086A02 | 1313 | PA4413 | 5187 | #N/A | #N/A |
| P1M10000086B01 | 1314 | PA4158 | 5157 | #N/A | #N/A |
| P1M10000086D02 | 1315 | PA2641 | 5115 | #N/A | #N/A |
| P1M10000086E05 | 1316 | PA3006 | 5121 | #N/A | #N/A |
| P1M10000087A11 | 1317 | PA4268 | 5178 | #N/A | #N/A |
| P1M10000087C09 | 1318 | PA2083 | 5097 | #N/A | #N/A |
| P1M10000087E04 | 1319 | PA4246 | 5162 | #N/A | #N/A |
| P1M10000087F04 | 1320 | PA0141 | 5056 | #N/A | #N/A |
| P1M10000087F09 | 1321 | PA4124 | 5155 | #N/A | #N/A |
| P1M10000087F09 | 1321 | PA4125 | 5156 | #N/A | #N/A |
| P1M10000088A07 | 1322 | PA2742 | 5120 | #N/A | #N/A |
| P1M10000088D06 | 1323 | PA2108 | 5099 | #N/A | #N/A |
| P1M10000089C08 | 1324 | PA3048 | 5125 | #N/A | #N/A |
| P1M10000089D11 | 1325 | PA4268 | 5178 | #N/A | #N/A |
| P1M10000089G08 | 1326 | PA2461 | 5108 | #N/A | #N/A |
| P1M10000090B11 | 1327 | PA3153 | 5128 | #N/A | #N/A |
| P1M10000090F06 | 1328 | PA2313 | 5105 | #N/A | #N/A |
| P1M10000090F08 | 1329 | PA4258 | 5173 | #N/A | #N/A |
| P1M10000090F08 | 1329 | PA4259 | 5174 | #N/A | #N/A |
| P1M10000091D02 | 1330 | PA3866 | 5143 | #N/A | #N/A |
| P1M10000091E09 | 1331 | PA5316 | 5212 | #N/A | #N/A |
| P1M10000091G10 | 1332 | PA2742 | 5120 | #N/A | #N/A |
| P1M10000092B02 | 1333 | PA2641 | 5115 | #N/A | #N/A |
| P1M10000092B10 | 1334 | PA4268 | 5178 | #N/A | #N/A |
| P1M10000092D09 | 1335 | PA2128 | 5100 | #N/A | #N/A |
| P1M10000092E02 | 1336 | PA4256 | 5171 | #N/A | #N/A |
| P1M10000092F05 | 1337 | PA0423 | 5067 | #N/A | #N/A |
| P1M10000093A03 | 1338 | PA5088 | 5205 | #N/A | #N/A |
| P1M10000093B09 | 1339 | PA3703 | 5138 | #N/A | #N/A |
| P1M10000093C08 | 1340 | PA1868 | 5092 | #N/A | #N/A |
| P1M10000093E09 | 1341 | PA4332 | 5183 | #N/A | #N/A |
| P1M10000093F03 | 1342 | PA2101 | 5098 | #N/A | #N/A |
| P1M10000093H07 | 1343 | PA4665 | 5195 | #N/A | #N/A |
| P1M10000094F04 | 1344 | PA4268 | 5178 | #N/A | #N/A |
| P1M10000094H03 | 1345 | PA4744 | 5198 | #N/A | #N/A |
| P1M10000095C01 | 1346 | PA2488 | 5110 | #N/A | #N/A |
| P1M10000095C09 | 1347 | PA5443 | 5216 | #N/A | #N/A |
| P1M10000095E04 | 1348 | PA4363 | 5185 | #N/A | #N/A |
| P1M10000095G04 | 1349 | PA4256 | 5171 | #N/A | #N/A |
| P1M10000096E04 | 1350 | PA0353 | 5061 | #N/A | #N/A |
| P1M10000096E12 | 1351 | PA4246 | 5162 | #N/A | #N/A |
| S1M10000001A05 | 1354 | SAU103232 | 5769 | SAU1c0045_orf_341p | 12697 |
| S1M10000001A05 | 1354 | SAU201508 | 5819 | SAU2c0432_orf_19p | 12947 |
| S1M10000001A08 | 1355 | SAU102437 | 5670 | SAU1c0045_orf_33p | 12695 |
| S1M10000001A09 | 1356 | SAU101907 | 5574 | SAU1c0040_orf_79p | 12442 |
| S1M10000001A10 | 1357 | SAU102602 | 5708 | SAU1c0032_orf_5p | 12249 |
| S1M10000001C06 | 1358 | SAU102939 | 5747 | #N/A | #N/A |
| S1M10000001D01 | 1359 | SAU101907 | 5574 | SAU1c0040_orf_79p | 12442 |
| S1M10000001D02 | 1360 | SAU100527 | 5285 | SAU1c0037_orf_101p | 12341 |
| S1M10000001D02 | 1360 | SAU100880 | 5346 | SAU1c0037_orf_100p | 12340 |
| S1M10000001D06 | 1361 | SAU101632 | 5499 | SAU1c0039_orf_3p | 12407 |
| S1M10000001D07 | 1362 | SAU101360 | 5431 | SAU1c0044_orf_109p | 12555 |
| S1M10000001E02 | 1363 | SAU102602 | 5708 | SAU1c0032_orf_5p | 12249 |
| S1M10000001E04 | 1364 | SAU102284 | 5635 | SAU1c0038_orf_5p | 12389 |
| S1M10000001E04 | 1364 | SAU201469 | 5816 | SAU2c0438_orf_6p | 12967 |
| S1M10000001E05 | 1365 | SAU102939 | 5747 | #N/A | #N/A |
| S1M10000001E09 | 1366 | SAU201752 | 5832 | SAU2c0436_orf_19p | 12963 |
| S1M10000001E10 | 1367 | SAU103038 | 5757 | #N/A | #N/A |
| S1M10000001E11 | 1368 | SAU302513 | 5906 | SAU3c1298_orf_1p | 13085 |
| S1M10000001F02 | 1369 | SAU101907 | 5574 | SAU1c0040_orf_79p | 12442 |
| S1M10000001F04 | 1370 | SAU100300 | 5253 | SAU1c0040_orf_90p | 12451 |
| S1M10000001F08 | 1371 | SAU102437 | 5670 | SAU1c0045_orf_33p | 12695 |
| S1M10000001F09 | 1372 | SAU101907 | 5574 | SAU1c0040_orf_79p | 12442 |
| S1M10000001F10 | 1373 | SAU102602 | 5708 | SAU1c0032_orf_5p | 12249 |
| S1M10000001F11 | 1374 | SAU102939 | 5747 | #N/A | #N/A |
| S1M10000001G01 | 1375 | SAU102939 | 5747 | #N/A | #N/A |
| S1M10000001G07 | 1376 | SAU102939 | 5747 | #N/A | #N/A |
| S1M10000001G08 | 1377 | SAU102939 | 5747 | #N/A | #N/A |
| S1M10000001G10 | 1378 | SAU100300 | 5253 | SAU1c0040_orf_90p | 12451 |
| S1M10000002A02 | 1379 | SAU102631 | 5721 | SAU1c0045_orf_94p | 12712 |
| S1M10000002A09 | 1380 | SAU101495 | 5467 | SAU1c0037_orf_65p | 12360 |
| S1M10000002A10 | 1381 | SAU201810 | 5836 | SAU2c0308_orf_2p | 12769 |
| S1M10000002A10 | 1381 | SAU202174 | 5845 | SAU2c0412_orf_3p | 12895 |
| S1M10000002A10 | 1381 | SAU301148 | 5888 | #N/A | #N/A |
| S1M10000002A12 | 1382 | SAU200916 | 5797 | SAU2c0373_orf_4p | 12838 |
| S1M10000002A12 | 1382 | SAU300455 | 5872 | #N/A | #N/A |
| S1M10000002A12 | 1382 | SAU301620 | 5899 | SAU3c1478_orf_2p | 13140 |
| S1M10000002B01 | 1383 | SAU102602 | 5708 | SAU1c0032_orf_5p | 12249 |
| S1M10000002B03 | 1384 | SAU101034 | 5371 | SAU1c0044_orf_27p | 12608 |
| S1M10000002B04 | 1385 | SAU101907 | 5574 | SAU1c0040_orf_79p | 12442 |
| S1M10000002B05 | 1386 | SAU101868 | 5565 | SAU1c0036_orf_23p | 12320 |
| S1M10000002B06 | 1387 | SAU100157 | 5237 | SAU1c0040_orf_81p | 12444 |
| S1M10000002B07 | 1388 | SAU101389 | 5441 | SAU1c0038_orf_54p | 12387 |
| S1M10000002B09 | 1389 | SAU201810 | 5836 | SAU2c0308_orf_2p | 12769 |
| S1M10000002B09 | 1389 | SAU202174 | 5845 | SAU2c0412_orf_3p | 12895 |
| S1M10000002B09 | 1389 | SAU301148 | 5888 | #N/A | #N/A |
| S1M10000002B11 | 1390 | SAU100521 | 5283 | SAU1c0044_orf_250p | 12600 |
| S1M10000002C02 | 1391 | SAU201810 | 5836 | SAU2c0308_orf_2p | 12769 |
| S1M10000002C02 | 1391 | SAU202174 | 5845 | SAU2c0412_orf_3p | 12895 |
| S1M10000002C02 | 1391 | SAU301148 | 5888 | #N/A | #N/A |
| S1M10000002C09 | 1392 | SAU101752 | 5522 | SAU1c0040_orf_85p | 12447 |
| S1M10000002C10 | 1393 | SAU103077 | 5759 | SAU1c0039_orf_44p | 12408 |
| S1M10000002C11 | 1394 | SAU202267 | 5848 | SAU2c0204_orf_2p | 12727 |
| S1M10000002C11 | 1394 | SAU202781 | 5853 | SAU2c0109_orf_2p | 12718 |
| S1M10000002C11 | 1394 | SAU203001 | 5859 | SAU2c0412_orf_15p | 12894 |
| S1M10000002C11 | 1394 | SAU302698 | 5909 | SAU3c1408_orf_2p | 13114 |
| S1M10000002C11 | 1394 | SAU302699 | 5910 | SAU3c1408_orf_3p | 13115 |
| S1M10000002C12 | 1395 | SAU101039 | 5373 | SAU1c0043_orf_181p | 12522 |
| S1M10000002D01 | 1396 | SAU101907 | 5574 | SAU1c0040_orf_79p | 12442 |
| S1M10000002D02 | 1397 | SAU100741 | 5318 | SAU1c0039_orf_48p | 12409 |
| S1M10000002D03 | 1398 | SAU102631 | 5721 | SAU1c0045_orf_94p | 12712 |
| S1M10000002D05 | 1399 | SAU202930 | 5856 | SAU2c0396_orf_3p | 12871 |
| S1M10000002D07 | 1400 | SAU101652 | 5503 | SAU1c0042_orf_123p | 12492 |
| S1M10000002D07 | 1400 | SAU101653 | 5504 | SAU1c0042_orf_124p | 12493 |
| S1M10000002D08 | 1401 | SAU101652 | 5503 | SAU1c0042_orf_123p | 12492 |
| S1M10000002D10 | 1402 | SAU102939 | 5747 | #N/A | #N/A |
| S1M10000002D12 | 1403 | SAU100952 | 5358 | SAU1c0043_orf_182p | 12523 |
| S1M10000002E01 | 1404 | SAU101616 | 5495 | SAU1c0040_orf_32p | 12432 |
| S1M10000002E02 | 1405 | SAU200914 | 5796 | SAU2c0373_orf_2p | 12837 |
| S1M10000002E07 | 1406 | SAU201810 | 5836 | SAU2c0308_orf_2p | 12769 |
| S1M10000002E07 | 1406 | SAU202174 | 5845 | SAU2c0412_orf_3p | 12895 |
| S1M10000002E07 | 1406 | SAU301148 | 5888 | #N/A | #N/A |
| S1M10000002E09 | 1407 | SAU100158 | 5238 | SAU1c0040_orf_80p | 12443 |
| S1M10000002E11 | 1408 | SAU102631 | 5721 | SAU1c0045_orf_94p | 12712 |
| S1M10000002E12 | 1409 | SAU101869 | 5566 | SAU1c0036_orf_24p | 12321 |
| S1M10000002F01 | 1410 | SAU200928 | 5798 | SAU2c0365_orf_5p | 12815 |
| S1M10000002F02 | 1411 | SAU301620 | 5899 | SAU3c1478_orf_2p | 13140 |
| S1M10000002F04 | 1412 | SAU102939 | 5747 | #N/A | #N/A |
| S1M10000002F09 | 1413 | SAU302513 | 5906 | SAU3c1298_orf_1p | 13085 |
| S1M10000002F12 | 1414 | SAU101907 | 5574 | SAU1c0040_orf_79p | 12442 |
| S1M10000002G01 | 1415 | SAU102939 | 5747 | #N/A | #N/A |
| S1M10000002G03 | 1416 | SAU100608 | 5297 | SAU1c0034_orf_69p | 12293 |
| S1M10000002G05 | 1417 | SAU101907 | 5574 | SAU1c0040_orf_79p | 12442 |
| S1M10000002G06 | 1418 | SAU101907 | 5574 | SAU1c0040_orf_79p | 12442 |
| S1M10000002G07 | 1419 | SAU103038 | 5757 | #N/A | #N/A |
| S1M10000002G08 | 1420 | SAU100158 | 5238 | SAU1c0040_orf_80p | 12443 |
| S1M10000002G09 | 1421 | SAU102939 | 5747 | #N/A | #N/A |
| S1M10000002G10 | 1422 | SAU101495 | 5467 | SAU1c0037_orf_65p | 12360 |
| S1M10000002G11 | 1423 | SAU102939 | 5747 | #N/A | #N/A |
| S1M10000002G12 | 1424 | SAU101907 | 5574 | SAU1c0040_orf_79p | 12442 |
| S1M10000003A01 | 1425 | SAU201810 | 5836 | SAU2c0308_orf_2p | 12769 |
| S1M10000003A01 | 1425 | SAU202174 | 5845 | SAU2c0412_orf_3p | 12895 |
| S1M10000003A01 | 1425 | SAU301148 | 5888 | #N/A | #N/A |
| S1M10000003A02 | 1426 | SAU101624 | 5497 | SAU1c0040_orf_25p | 12429 |
| S1M10000003A03 | 1427 | SAU101752 | 5522 | SAU1c0040_orf_85p | 12447 |
| S1M10000003A04 | 1428 | SAU101360 | 5431 | SAU1c0044_orf_109p | 12555 |
| S1M10000003A06 | 1429 | SAU101266 | 5408 | SAU1c0042_orf_117p | 12490 |
| S1M10000003A07 | 1430 | SAU101907 | 5574 | SAU1c0040_orf_79p | 12442 |
| S1M10000003A08 | 1431 | SAU102939 | 5747 | #N/A | #N/A |
| S1M10000003A10 | 1432 | SAU100432 | 5271 | SAU1c0040_orf_88p | 12450 |
| S1M10000003A11 | 1433 | SAU101495 | 5467 | SAU1c0037_orf_65p | 12360 |
| S1M10000003B06 | 1434 | SAU102007 | 5590 | SAU1c0040_orf_108p | 12428 |
| S1M10000003B08 | 1435 | SAU100952 | 5358 | SAU1c0043_orf_182p | 12523 |
| S1M10000003B09 | 1436 | SAU100771 | 5325 | SAU1c0043_orf_49p | 12545 |
| S1M10000003B12 | 1437 | SAU302060 | 5905 | SAU3c0879_orf_1p | 13042 |
| S1M10000003C06 | 1438 | SAU102447 | 5672 | SAU1c0045_orf_24p | 12685 |
| S1M10000003C07 | 1439 | SAU101271 | 5411 | SAU1c0037_orf_90p | 12366 |
| S1M10000003C10 | 1440 | SAU101907 | 5574 | SAU1c0040_orf_79p | 12442 |
| S1M10000003C12 | 1441 | SAU101907 | 5574 | SAU1c0040_orf_79p | 12442 |
| S1M10000003D05 | 1442 | SAU102939 | 5747 | #N/A | #N/A |
| S1M10000003D06 | 1443 | SAU101996 | 5584 | SAU1c0040_orf_99p | 12456 |
| S1M10000003D08 | 1444 | SAU100793 | 5329 | SAU1c0028_orf_52p | 12188 |
| S1M10000003D10 | 1445 | SAU102422 | 5666 | SAU1c0030_orf_22p | 12207 |
| S1M10000003E07 | 1446 | SAU100964 | 5363 | SAU1c0044_orf_86p | 12641 |
| S1M10000003E09 | 1447 | SAU101907 | 5574 | SAU1c0040_orf_79p | 12442 |
| S1M10000003E10 | 1448 | SAU101674 | 5508 | SAU1c0044_orf_226p | 12594 |
| S1M10000003E11 | 1449 | SAU101907 | 5574 | SAU1c0040_orf_79p | 12442 |
| S1M10000003F02 | 1450 | SAU101907 | 5574 | SAU1c0040_orf_79p | 12442 |
| S1M10000003F05 | 1451 | SAU101092 | 5381 | SAU1c0028_orf_9p | 12192 |
| S1M10000003F06 | 1452 | SAU100158 | 5238 | SAU1c0040_orf_80p | 12443 |
| S1M10000003F07 | 1453 | SAU200914 | 5796 | SAU2c0373_orf_2p | 12837 |
| S1M10000003F08 | 1454 | SAU102939 | 5747 | #N/A | #N/A |
| S1M10000003F12 | 1455 | SAU101360 | 5431 | SAU1c0044_orf_109p | 12555 |
| S1M10000003G03 | 1456 | SAU101907 | 5574 | SAU1c0040_orf_79p | 12442 |
| S1M10000003G04 | 1457 | SAU201810 | 5836 | SAU2c0308_orf_2p | 12769 |
| S1M10000003G04 | 1457 | SAU202174 | 5845 | SAU2c0412_orf_3p | 12895 |
| S1M10000003G04 | 1457 | SAU301148 | 5888 | #N/A | #N/A |
| S1M10000003G08 | 1458 | SAU102939 | 5747 | #N/A | #N/A |
| S1M10000003G10 | 1459 | SAU102939 | 5747 | #N/A | #N/A |
| S1M10000004A04 | 1460 | SAU102631 | 5721 | SAU1c0045_orf_94p | 12712 |
| S1M10000004A06 | 1461 | SAU100964 | 5363 | SAU1c0044_orf_86p | 12641 |
| S1M10000004A07 | 1462 | SAU200916 | 5797 | SAU2c0373_orf_4p | 12838 |
| S1M10000004A11 | 1463 | SAU100521 | 5283 | SAU1c0044_orf_250p | 12600 |
| S1M10000004A12 | 1464 | SAU102132 | 5605 | SAU1c0027_orf_19p | 12177 |
| S1M10000004B03 | 1465 | SAU102610 | 5714 | SAU1c0041_orf_53p | 12474 |
| S1M10000004B04 | 1466 | SAU102059 | 5597 | SAU1c0034_orf_51p | 1286 |
| S1M10000004B06 | 1467 | SAU102939 | 5747 | #N/A | #N/A |
| S1M10000004B08 | 1468 | SAU100272 | 5251 | SAU1c0018_orf_7p | 12141 |
| S1M10000004B09 | 1469 | SAU101476 | 5459 | SAU1c0032_orf_69p | 12254 |
| S1M10000004B11 | 1470 | SAU101495 | 5467 | SAU1c0037_orf_65p | 12360 |
| S1M10000004C01 | 1471 | SAU102631 | 5721 | SAU1c0045_orf_94p | 12712 |
| S1M10000004C02 | 1472 | SAU201810 | 5836 | SAU2c0308_orf_2p | 12769 |
| S1M10000004C02 | 1472 | SAU202174 | 5845 | SAU2c0412_orf_3p | 12895 |
| S1M10000004C02 | 1472 | SAU301148 | 5888 | #N/A | #N/A |
| S1M10000004C03 | 1473 | SAU102939 | 5747 | #N/A | #N/A |
| S1M10000004C06 | 1474 | SAU102883 | 5741 | SAU1c0045_orf_38p | 12702 |
| S1M10000004C07 | 1475 | SAU102939 | 5747 | #N/A | #N/A |
| S1M10000004C08 | 1476 | SAU101455 | 5456 | SAU1c0045_orf_250p | 12686 |
| S1M10000004C08 | 1476 | SAU200916 | 5797 | SAU2c0373_orf_4p | 12838 |
| S1M10000004C09 | 1477 | SAU201810 | 5836 | SAU2c0308_orf_2p | 12769 |
| S1M10000004C09 | 1477 | SAU202174 | 5845 | SAU2c0412_orf_3p | 12895 |
| S1M10000004C09 | 1477 | SAU301148 | 5888 | #N/A | #N/A |
| S1M10000004C10 | 1478 | SAU101271 | 5411 | SAU1c0037_orf_90p | 12366 |
| S1M10000004C10 | 1478 | SAU101286 | 5413 | SAU1c0034_orf_67p | 12292 |
| S1M10000004C10 | 1478 | SAU302931 | 5913 | SAU3c1507_orf_10p | 13155 |
| S1M10000004C12 | 1479 | SAU102007 | 5590 | SAU1c0040_orf_108p | 12428 |
| S1M10000004D01 | 1480 | SAU101301 | 5416 | SAU1c0044_orf_114p | 12558 |
| S1M10000004D01 | 1480 | SAU101302 | 5417 | SAU1c0044_orf_115p | 12559 |
| S1M10000004D03 | 1481 | SAU102390 | 5657 | SAU1c0033_orf_38p | 12269 |
| S1M10000004D03 | 1481 | SAU201333 | 5810 | SAU2c0418_orf_8p | 12905 |
| S1M10000004D04 | 1482 | SAU101807 | 5547 | SAU1c0032_orf_26p | 12231 |
| S1M10000004D04 | 1482 | SAU101808 | 5548 | SAU1c0032_orf_27p | 12232 |
| S1M10000004D06 | 1483 | SAU201571 | 5824 | SAU2c0447_orf_17p | 12997 |
| S1M10000004D07 | 1484 | SAU201810 | 5836 | SAU2c0308_orf_2p | 12769 |
| S1M10000004D07 | 1484 | SAU202174 | 5845 | SAU2c0412_orf_3p | 12895 |
| S1M10000004D07 | 1484 | SAU301148 | 5888 | #N/A | #N/A |
| S1M10000004D08 | 1485 | SAU100414 | 5270 | SAU1c0022_orf_24p | 12148 |
| S1M10000004D10 | 1486 | SAU101365 | 5432 | SAU1c0044_orf_112p | 12556 |
| S1M10000004D12 | 1487 | SAU101545 | 5474 | SAU1c0037_orf_132p | 12348 |
| S1M10000004D12 | 1487 | SAU101546 | 5475 | SAU1c0037_orf_133p | 12349 |
| S1M10000004E03 | 1488 | SAU101371 | 5435 | SAU1c0033_orf_7p | 12275 |
| S1M10000004E04 | 1489 | SAU102602 | 5708 | SAU1c0032_orf_5p | 12249 |
| S1M10000004E06 | 1490 | SAU101791 | 5532 | SAU1c0032_orf_12p | 12216 |
| S1M10000004E07 | 1491 | SAU101476 | 5459 | SAU1c0032_orf_69p | 12254 |
| S1M10000004E11 | 1492 | SAU102939 | 5747 | #N/A | #N/A |
| S1M10000004E12 | 1493 | SAU101996 | 5584 | SAU1c0040_orf_99p | 12456 |
| S1M10000004F01 | 1494 | SAU101039 | 5373 | SAU1c0043_orf_181p | 12522 |
| S1M10000004F02 | 1495 | SAU100157 | 5237 | SAU1c0040_orf_81p | 12444 |
| S1M10000004F06 | 1496 | SAU201611 | 5825 | SAU2c0440_orf_14p | 12973 |
| S1M10000004F07 | 1497 | SAU102764 | 5734 | SAU1c0044_orf_56p | 12625 |
| S1M10000004F08 | 1498 | SAU101807 | 5547 | SAU1c0032_orf_26p | 12231 |
| S1M10000004F08 | 1498 | SAU101808 | 5548 | SAU1c0032_orf_27p | 12232 |
| S1M10000004F09 | 1499 | SAU201810 | 5836 | SAU2c0308_orf_2p | 12769 |
| S1M10000004F09 | 1499 | SAU202174 | 5845 | SAU2c0412_orf_3p | 12895 |
| S1M10000004F09 | 1499 | SAU301148 | 5888 | #N/A | #N/A |
| S1M10000004F12 | 1500 | SAU101652 | 5503 | SAU1c0042_orf_123p | 12492 |
| S1M10000004G01 | 1501 | SAU201810 | 5836 | SAU2c0308_orf_2p | 12769 |
| S1M10000004G01 | 1501 | SAU202174 | 5845 | SAU2c0412_orf_3p | 12895 |
| S1M10000004G01 | 1501 | SAU301148 | 5888 | #N/A | #N/A |
| S1M10000004G02 | 1502 | SAU102939 | 5747 | #N/A | #N/A |
| S1M10000004G03 | 1503 | SAU102449 | 5674 | SAU1c0045_orf_22p | 12677 |
| S1M10000004G05 | 1504 | SAU101907 | 5574 | SAU1c0040_orf_79p | 12442 |
| S1M10000004G06 | 1505 | SAU102939 | 5747 | #N/A | #N/A |
| S1M10000004G07 | 1506 | SAU100964 | 5363 | SAU1c0044_orf_86p | 12641 |
| S1M10000004G07 | 1506 | SAU100965 | 5364 | SAU1c0044_orf_57p | 12642 |
| S1M10000004G09 | 1507 | SAU101869 | 5566 | SAU1c0036_orf_24p | 12321 |
| S1M10000004G12 | 1508 | SAU100497 | 5280 | SAU1c0018_orf_3p | 12140 |
| S1M10000005A01 | 1509 | SAU201810 | 5836 | SAU2c0308_orf_2p | 12769 |
| S1M10000005A01 | 1509 | SAU202174 | 5845 | SAU2c0412_orf_3p | 12895 |
| S1M10000005A01 | 1509 | SAU301148 | 5888 | #N/A | #N/A |
| S1M10000005A03 | 1510 | SAU101090 | 5380 | SAU1c0028_orf_8p | 12191 |
| S1M10000005A05 | 1511 | SAU102939 | 5747 | #N/A | #N/A |
| S1M10000005A06 | 1512 | SAU102939 | 5747 | #N/A | #N/A |
| S1M10000005A07 | 1513 | SAU100952 | 5358 | SAU1c0043_orf_182p | 12523 |
| S1M10000005A08 | 1514 | SAU201810 | 5836 | SAU2c0308_orf_2p | 12769 |
| S1M10000005A08 | 1514 | SAU202174 | 5845 | SAU2c0412_orf_3p | 12895 |
| S1M10000005A08 | 1514 | SAU301148 | 5888 | #N/A | #N/A |
| S1M10000005A09 | 1515 | SAU103038 | 5757 | #N/A | #N/A |
| S1M10000005A10 | 1516 | SAU101239 | 5402 | SAU1c0044_orf_15p | 12570 |
| S1M10000005A10 | 1516 | SAU101240 | 5403 | SAU1c0044_orf_16p | 12573 |
| S1M10000005A11 | 1517 | SAU100964 | 5363 | SAU1c0044_orf_86p | 12641 |
| S1M10000005B02 | 1518 | SAU102527 | 5693 | SAU1c0032_orf_9p | 12260 |
| S1M10000005B04 | 1519 | SAU101545 | 5474 | SAU1c0037_orf_132p | 12348 |
| S1M10000005B07 | 1520 | SAU201810 | 5836 | SAU2c0308_orf_2p | 12769 |
| S1M10000005B07 | 1520 | SAU202174 | 5845 | SAU2c0412_orf_3p | 12895 |
| S1M10000005B07 | 1520 | SAU301148 | 5888 | #N/A | #N/A |
| S1M10000005B08 | 1521 | SAU101907 | 5574 | SAU1c0040_orf_79p | 12442 |
| S1M10000005B09 | 1522 | SAU102422 | 5666 | SAU1c0030_orf_22p | 12207 |
| S1M10000005B12 | 1523 | SAU102284 | 5635 | SAU1c0038_orf_5p | 12389 |
| S1M10000005B12 | 1523 | SAU201469 | 5816 | SAU2c0438_orf_6p | 12967 |
| S1M10000005C01 | 1524 | SAU201810 | 5836 | SAU2c0308_orf_2p | 12769 |
| S1M10000005C01 | 1524 | SAU202174 | 5845 | SAU2c0412_orf_3p | 12895 |
| S1M10000005C01 | 1524 | SAU301148 | 5888 | #N/A | #N/A |
| S1M10000005C05 | 1525 | SAU101869 | 5566 | SAU1c0036_orf_24p | 12321 |
| S1M10000005C06 | 1526 | SAU100885 | 5348 | SAU1c0038_orf_38p | 12376 |
| S1M10000005C09 | 1527 | SAU302513 | 5906 | SAU3c1298_orf_1p | 13085 |
| S1M10000005C11 | 1528 | SAU101495 | 5467 | SAU1c0037_orf_65p | 12360 |
| S1M10000005D01 | 1529 | SAU103038 | 5757 | #N/A | #N/A |
| S1M10000005D02 | 1530 | SAU102007 | 5590 | SAU1c0040_orf_108p | 12428 |
| S1M10000005D03 | 1531 | SAU101907 | 5574 | SAU1c0040_orf_79p | 12442 |
| S1M10000005D04 | 1532 | SAU101545 | 5474 | SAU1c0037_orf_132p | 12348 |
| S1M10000005D04 | 1532 | SAU101546 | 5475 | SAU1c0037_orf_133p | 12349 |
| S1M10000005D05 | 1533 | SAU100964 | 5363 | SAU1c0044_orf_86p | 12641 |
| S1M10000005D06 | 1534 | SAU101545 | 5474 | SAU1c0037_orf_132p | 12348 |
| S1M10000005D06 | 1534 | SAU101546 | 5475 | SAU1c0037_orf_133p | 12349 |
| S1M10000005D07 | 1535 | SAU101869 | 5566 | SAU1c0036_orf_24p | 12321 |
| S1M10000005D08 | 1536 | SAU101624 | 5497 | SAU1c0040_orf_25p | 12429 |
| S1M10000005D09 | 1537 | SAU101752 | 5522 | SAU1c0040_orf_85p | 12447 |
| S1M10000005D11 | 1538 | SAU100158 | 5238 | SAU1c0040_orf_80p | 12443 |
| S1M10000005D12 | 1539 | SAU100964 | 5363 | SAU1c0044_orf_86p | 12641 |
| S1M10000005E01 | 1540 | SAU100542 | 5288 | SAU1c0043_orf_210p | 12532 |
| S1M10000005E02 | 1541 | SAU102631 | 5721 | SAU1c0045_orf_94p | 12712 |
| S1M10000005E05 | 1542 | SAU201810 | 5836 | SAU2c0308_orf_2p | 12769 |
| S1M10000005E05 | 1542 | SAU202174 | 5845 | SAU2c0412_orf_3p | 12895 |
| S1M10000005E05 | 1542 | SAU301148 | 5888 | #N/A | #N/A |
| S1M10000005E06 | 1543 | SAU102939 | 5747 | #N/A | #N/A |
| S1M10000005E07 | 1544 | SAU102939 | 5747 | #N/A | #N/A |
| S1M10000005E08 | 1545 | SAU201810 | 5836 | SAU2c0308_orf_2p | 12769 |
| S1M10000005E08 | 1545 | SAU202174 | 5845 | SAU2c0412_orf_3p | 12895 |
| S1M10000005E08 | 1545 | SAU301148 | 5888 | #N/A | #N/A |
| S1M10000005E10 | 1546 | SAU102939 | 5747 | #N/A | #N/A |
| S1M10000005E11 | 1547 | SAU100381 | 5265 | SAU1c0033_orf_9p | 12276 |
| S1M10000005E12 | 1548 | SAU102939 | 5747 | #N/A | #N/A |
| S1M10000005F02 | 1549 | SAU100964 | 5363 | SAU1c0044_orf_86p | 12641 |
| S1M10000005F02 | 1549 | SAU100965 | 5364 | SAU1c0044_orf_87p | 12642 |
| S1M10000005F03 | 1550 | SAU100793 | 5329 | SAU1c0028_orf_52p | 12188 |
| S1M10000005F03 | 1550 | SAU301433 | 5895 | SAU3c1420_orf_2p | 13118 |
| S1M10000005F04 | 1551 | SAU102044 | 5593 | SAU1c0039_orf_65p | 12414 |
| S1M10000005F04 | 1551 | SAU102046 | 5594 | SAU1c0039_orf_66p | 12415 |
| S1M10000005F04 | 1551 | SAU201961 | 5840 | #N/A | #N/A |
| S1M10000006A03 | 1552 | SAU201810 | 5836 | SAU2c0308_orf_2p | 12769 |
| S1M10000006A03 | 1552 | SAU202174 | 5845 | SAU2c0412_orf_3p | 12895 |
| S1M10000006A03 | 1552 | SAU301148 | 5888 | #N/A | #N/A |
| S1M10000006A04 | 1553 | SAU101271 | 5411 | SAU1c0037_orf_90p | 12366 |
| S1M10000006A05 | 1554 | SAU101807 | 5547 | SAU1c0032_orf_26p | 12231 |
| S1M10000006A05 | 1554 | SAU101808 | 5548 | SAU1c0032_orf_27p | 12232 |
| S1M10000006A07 | 1555 | SAU100952 | 5358 | SAU1c0043_orf_182p | 12523 |
| S1M10000006A08 | 1556 | SAU201810 | 5836 | SAU2c0308_orf_2p | 12769 |
| S1M10000006A08 | 1556 | SAU202174 | 5845 | SAU2c0412_orf_3p | 12895 |
| S1M10000006A08 | 1556 | SAU301148 | 5888 | #N/A | #N/A |
| S1M10000006A10 | 1557 | SAU201810 | 5836 | SAU2c0308_orf_2p | 12769 |
| S1M10000006A10 | 1557 | SAU202174 | 5845 | SAU2c0412_orf_3p | 12895 |
| S1M10000006A10 | 1557 | SAU301148 | 5888 | #N/A | #N/A |
| S1M10000006A12 | 1558 | SAU101907 | 5574 | SAU1c0040_orf_79p | 12442 |
| S1M10000006B02 | 1559 | SAU100741 | 5318 | SAU1c0039_orf_48p | 12409 |
| S1M10000006B03 | 1560 | SAU102631 | 5721 | SAU1c0045_orf_94p | 12712 |
| S1M10000006B04 | 1561 | SAU201810 | 5836 | SAU2c0308_orf_2p | 12769 |
| S1M10000006B04 | 1561 | SAU202174 | 5845 | SAU2c0412_orf_3p | 12895 |
| S1M10000006B04 | 1561 | SAU301148 | 5888 | #N/A | #N/A |
| S1M10000006B07 | 1562 | SAU102059 | 5597 | SAU1c0034_orf_51p | 1286 |
| S1M10000006B10 | 1563 | SAU101791 | 5532 | SAU1c0032_orf_12p | 12216 |
| S1M10000006B11 | 1564 | SAU101365 | 5432 | SAU1c0044_orf_112p | 12556 |
| S1M10000006C02 | 1565 | SAU102939 | 5747 | #N/A | #N/A |
| S1M10000006C04 | 1566 | SAU102287 | 5637 | SAU1c0038_orf_7p | 12398 |
| S1M10000006C06 | 1567 | SAU102486 | 5687 | SAU1c0039_orf_93p | 12420 |
| S1M10000006C06 | 1567 | SAU102487 | 5688 | SAU1c0039_orf_92p | 12419 |
| S1M10000006C07 | 1568 | SAU100157 | 5237 | SAU1c0040_orf_81p | 12444 |
| S1M10000006C08 | 1569 | SAU102939 | 5747 | #N/A | #N/A |
| S1M10000006C10 | 1570 | SAU201810 | 5836 | SAU2c0308_orf_2p | 12769 |
| S1M10000006C10 | 1570 | SAU202174 | 5845 | SAU2c0412_orf_3p | 12895 |
| S1M10000006C10 | 1570 | SAU301148 | 5888 | #N/A | #N/A |
| S1M10000006D03 | 1571 | SAU100608 | 5297 | SAU1c0034_orf_69p | 12293 |
| S1M10000006D05 | 1572 | SAU201810 | 5836 | SAU2c0308_orf_2p | 12769 |
| S1M10000006D05 | 1572 | SAU202174 | 5845 | SAU2c0412_orf_3p | 12895 |
| S1M10000006D05 | 1572 | SAU301148 | 5888 | #N/A | #N/A |
| S1M10000006D06 | 1573 | SAU201810 | 5836 | SAU2c0308_orf_2p | 12769 |
| S1M10000006D06 | 1573 | SAU202174 | 5845 | SAU2c0412_orf_3p | 12895 |
| S1M10000006D06 | 1573 | SAU301148 | 5888 | #N/A | #N/A |
| S1M10000006D07 | 1574 | SAU102936 | 5746 | SAU1c0037_orf_57p | 12356 |
| S1M10000006D08 | 1575 | SAU102939 | 5747 | #N/A | #N/A |
| S1M10000006E02 | 1576 | SAU201810 | 5836 | SAU2c0308_orf_2p | 12769 |
| S1M10000006E02 | 1576 | SAU202174 | 5845 | SAU2c0412_orf_3p | 12895 |
| S1M10000006E02 | 1576 | SAU301148 | 5888 | #N/A | #N/A |
| S1M10000006E03 | 1577 | SAU100275 | 5252 | SAU1c0036_orf_15p | 12314 |
| S1M10000006E04 | 1578 | SAU101777 | 5527 | SAU1c0037_orf_39p | 12352 |
| S1M10000006E07 | 1579 | SAU201810 | 5836 | SAU2c0308_orf_2p | 12769 |
| S1M10000006E07 | 1579 | SAU202174 | 5845 | SAU2c0412_orf_3p | 12895 |
| S1M10000006E07 | 1579 | SAU301148 | 5888 | #N/A | #N/A |
| S1M10000006E08 | 1580 | SAU101793 | 5534 | SAU1c0032_orf_14p | 12218 |
| S1M10000006F01 | 1581 | SAU101869 | 5566 | SAU1c0036_orf_24p | 12321 |
| S1M10000006F02 | 1582 | SAU201469 | 5816 | SAU2c0438_orf_6p | 12967 |
| S1M10000006F03 | 1583 | SAU102294 | 5639 | SAU1c0044_orf_288p | 12610 |
| S1M10000006F03 | 1583 | SAU301080 | 5885 | SAU3c1287_orf_1p | 13083 |
| S1M10000006F04 | 1584 | SAU100964 | 5363 | SAU1c0044_orf_86p | 12641 |
| S1M10000006F06 | 1585 | SAU101907 | 5574 | SAU1c0040_orf_79p | 12442 |
| S1M10000006G02 | 1586 | SAU101833 | 5555 | SAU1c0038_orf_34p | 12373 |
| S1M10000006G03 | 1587 | SAU101400 | 5444 | SAU1c0036_orf_35p | 12326 |
| S1M10000006G05 | 1588 | SAU100275 | 5252 | SAU1c0036_orf_15p | 12314 |
| S1M10000006G06 | 1589 | SAU201571 | 5824 | SAU2c0447_orf_17p | 12997 |
| S1M10000006G07 | 1590 | SAU101612 | 5493 | SAU1c0044_orf_7p | 12637 |
| S1M10000006G07 | 1590 | SAU202945 | 5857 | SAU2c0394_orf_7p | 12868 |
| S1M10000006G09 | 1591 | SAU102939 | 5747 | #N/A | #N/A |
| S1M10000006G10 | 1592 | SAU102602 | 5708 | SAU1c0032_orf_5p | 12249 |
| S1M10000006G11 | 1593 | SAU101438 | 5450 | SAU1c0038_orf_40p | 12379 |
| S1M10000007A02 | 1594 | SAU102939 | 5747 | #N/A | #N/A |
| S1M10000007A03 | 1595 | SAU101653 | 5504 | SAU1c0042_orf_124p | 12493 |
| S1M10000007B02 | 1596 | SAU102352 | 5650 | SAU1c0040_orf_38p | 12434 |
| S1M10000007B02 | 1596 | SAU202872 | 5854 | SAU2c0393_orf_6p | 12866 |
| S1M10000007B11 | 1597 | SAU101476 | 5459 | SAU1c0032_orf_69p | 12254 |
| S1M10000007C02 | 1598 | SAU102939 | 5747 | #N/A | #N/A |
| S1M10000007C04 | 1599 | SAU100608 | 5297 | SAU1c0034_orf_69p | 12293 |
| S1M10000007C05 | 1600 | SAU100158 | 5238 | SAU1c0040_orf_80p | 12443 |
| S1M10000007C06 | 1601 | SAU101652 | 5503 | SAU1c0042_orf_123p | 12492 |
| S1M10000007C07 | 1602 | SAU101266 | 5408 | SAU1c0042_orf_117p | 12490 |
| S1M10000007C08 | 1603 | SAU101717 | 5513 | SAU1c0016_orf_16p | 12131 |
| S1M10000007C09 | 1604 | SAU102939 | 5747 | 4N/A | #N/A |
| S1M10000007D03 | 1605 | SAU201810 | 5836 | SAU2c0308_orf_2p | 12769 |
| S1M10000007D03 | 1605 | SAU202174 | 5845 | SAU2c0412_orf_3p | 12895 |
| S1M10000007D03 | 1605 | SAU301148 | 5888 | #N/A | #N/A |
| S1M10000007D06 | 1606 | SAU100158 | 5238 | SAU1c0040_orf_80p | 12443 |
| S1M10000007D08 | 1607 | SAU102939 | 5747 | #N/A | #N/A |
| S1M10000007D10 | 1608 | SAU100300 | 5253 | SAU1c0040_orf_90p | 12451 |
| S1M10000007D11 | 1609 | SAU101652 | 5503 | SAU1c0042_orf_123p | 12492 |
| S1M10000007E04 | 1610 | SAU201810 | 5836 | SAU2c0308_orf_2p | 12769 |
| S1M10000007E04 | 1610 | SAU202174 | 5845 | SAU2c0412_orf_3p | 12895 |
| S1M10000007E04 | 1610 | SAU301148 | 5888 | #N/A | #N/A |
| S1M10000007E06 | 1611 | SAU101495 | 5467 | SAU1c0037_orf_65p | 12360 |
| S1M10000007E07 | 1612 | SAU101365 | 5432 | SAU1c0044_orf_112p | 12556 |
| S1M10000007F01 | 1613 | SAU100275 | 5252 | SAU1c0036_orf_15p | 12314 |
| S1M10000007F02 | 1614 | SAU101685 | 5512 | SAU1c0023_orf_11p | 12152 |
| S1M10000007F04 | 1615 | SAU101491 | 5464 | SAU1c0025_orf_20p | 12165 |
| S1M10000007F08 | 1616 | SAU100794 | 5330 | SAU1c0028_orf_53p | 12189 |
| S1M10000007F09 | 1617 | SAU202930 | 5856 | SAU2c0396_orf_3p | 12871 |
| S1M10000007F10 | 1618 | SAU101791 | 5532 | SAU1c0032_orf_12p | 12216 |
| S1M10000007F11 | 1619 | SAU102939 | 5747 | #N/A | #N/A |
| S1M10000007F12 | 1620 | SAU102939 | 5747 | #N/A | #N/A |
| S1M10000007G02 | 1621 | SAU101270 | 5410 | SAU1c0037_orf_89p | 12365 |
| S1M10000007G03 | 1622 | SAU100952 | 5358 | SAU1c0043_orf_182p | 12523 |
| S1M10000007G05 | 1623 | SAU101907 | 5574 | SAU1c0040_orf_79p | 12442 |
| S1M10000007G07 | 1624 | SAU102652 | 5725 | SAU1c0045_orf_115p | 12653 |
| S1M10000007G08 | 1625 | SAU103038 | 5757 | #N/A | #N/A |
| S1M10000008A03 | 1626 | SAU101476 | 5459 | SAU1c0032_orf_69p | 12254 |
| S1M10000008A04 | 1627 | SAU101491 | 5464 | SAU1c0025_orf_20p | 12165 |
| S1M10000008A05 | 1628 | SAU102939 | 5747 | #N/A | #N/A |
| S1M10000008A08 | 1629 | SAU102905 | 5742 | SAU1c0033_orf_45p | 12273 |
| S1M10000008A08 | 1629 | SAU301869 | 5903 | SAU3c1353_orf_1p | 13093 |
| S1M10000008A09 | 1630 | SAU100741 | 5318 | SAU1c0039_orf_48p | 12409 |
| S1M10000008A12 | 1631 | SAU100608 | 5297 | SAU1c0034_orf_69p | 12293 |
| S1M10000008B03 | 1632 | SAU103144 | 5761 | SAU1c0045_orf_15p | 12663 |
| S1M10000008B04 | 1633 | SAU201810 | 5836 | SAU2c0308_orf_2p | 12769 |
| S1M10000008B04 | 1633 | SAU202174 | 5845 | SAU2c0412_orf_3p | 12895 |
| S1M10000008B04 | 1633 | SAU301148 | 5888 | #N/A | #N/A |
| S1M10000008B06 | 1634 | SAU101806 | 5546 | SAU1c0032_orf_25p | 12230 |
| S1M10000008B08 | 1635 | SAU101652 | 5503 | SAU1c0042_orf_123p | 12492 |
| S1M10000008B09 | 1636 | SAU102117 | 5603 | SAU1c0027_orf_6p | 12181 |
| S1M10000008B10 | 1637 | SAU100608 | 5297 | SAU1c0034_orf_69p | 12293 |
| S1M10000008C05 | 1638 | SAU102939 | 5747 | #N/A | #N/A |
| S1M10000008C06 | 1639 | SAU102939 | 5747 | #N/A | #N/A |
| S1M10000008C07 | 1640 | SAU102939 | 5747 | #N/A | #N/A |
| S1M10000008C08 | 1641 | SAU201571 | 5824 | SAU2c0447_orf_17p | 12997 |
| S1M10000008C09 | 1642 | SAU101793 | 5534 | SAU1c0032_orf_14p | 12218 |
| S1M10000008D05 | 1643 | SAU100414 | 5270 | SAU1c0022_orf_24p | 12148 |
| S1M10000008D09 | 1644 | SAU103038 | 5757 | #N/A | #N/A |
| S1M10000008E05 | 1645 | SAU101545 | 5474 | SAU1c0037_orf_132p | 12348 |
| S1M10000008E08 | 1646 | SAU101907 | 5574 | SAU1c0040_orf_79p | 12442 |
| S1M10000008Eo9 | 1647 | SAU101343 | 5425 | SAU1c0044_orf_40p | 12619 |
| S1M10000008E10 | 1648 | SAU101360 | 5431 | SAU1c0044_orf_109p | 12555 |
| S1M10000008F01 | 1649 | SAU102284 | 5635 | SAU1c0038_orf_5p | 12389 |
| S1M10000008F01 | 1649 | SAU201469 | 5816 | SAU2c0438_orf_6p | 12967 |
| S1M10000008F02 | 1650 | SAU102007 | 5590 | SAU1c0040_orf_108p | 12428 |
| S1M10000008F03 | 1651 | SAU101028 | 5370 | SAU1c0043_orf_7p | 12552 |
| S1M10000008F06 | 1652 | SAU100741 | 5318 | SAU1c0039_orf_48p | 12409 |
| S1M10000008F08 | 1653 | SAU101365 | 5432 | SAU1c0044_orf_112p | 12556 |
| S1M10000008F09 | 1654 | SAU201810 | 5836 | SAU2c0308_orf_2p | 12769 |
| S1M10000008F09 | 1654 | SAU202174 | 5845 | SAU2c0412_orf_3p | 12895 |
| S1M10000008F09 | 1654 | SAU301148 | 5888 | #N/A | #N/A |
| S1M10000008F10 | 1655 | SAU100300 | 5253 | SAU1c0040_orf_90p | 12451 |
| S1M10000008F11 | 1656 | SAU301620 | 5899 | SAU3c1478_orf_2p | 13140 |
| S1M10000008G02 | 1657 | SAU201167 | 5803 | SAU2c0407_orf_5p | 12887 |
| S1M10000008G03 | 1658 | SAU101637 | 5500 | SAU1c0029_orf_8p | 12201 |
| S1M10000008G05 | 1659 | SAU102870 | 5738 | SAU1c0026_orf_17p | 12170 |
| S1M10000009A02 | 1660 | SAU101159 | 5387 | SAU1c0036_orf_46p | 12331 |
| S1M10000009A04 | 1661 | SAU102979 | 5750 | SAU1c0043_orf_227p | 12536 |
| S1M10000009A07 | 1662 | SAU101371 | 5435 | SAU1c0033_orf_7p | 12275 |
| S1M10000009A08 | 1663 | SAU100658 | 5303 | SAU1c0038_orf_59p | 12388 |
| S1M10000009A08 | 1663 | SAU100659 | 5304 | SAU1c0038_orf_60p | 12390 |
| S1M10000009A09 | 1664 | SAU201571 | 5824 | SAU2c0447_orf_17p | 12997 |
| S1M10000009A10 | 1665 | SAU100658 | 5303 | SAU1c0038_orf_59p | 12388 |
| S1M10000009A11 | 1666 | SAU100114 | 5228 | SAU1c0043_orf_225p | 12535 |
| S1M10000009B01 | 1667 | SAU201506 | 5818 | SAU2c0432_orf_18p | 12946 |
| S1M10000009B02 | 1668 | SAU101159 | 5387 | SAU1c0036_orf_46p | 12331 |
| S1M10000009B03 | 1669 | SAU201506 | 5818 | SAU2c0432_orf_18p | 12946 |
| S1M10000009B04 | 1670 | SAU102117 | 5603 | SAU1c0027_orf_6p | 12181 |
| S1M10000009B05 | 1671 | SAU101752 | 5522 | SAU1c0040_orf_85p | 12447 |
| S1M10000009B06 | 1672 | SAU101271 | 5411 | SAU1c0037_orf_90p | 12366 |
| S1M10000009B07 | 1673 | SAU201952 | 5839 | SAU2c0457_orf_10p | 13020 |
| S1M10000009B10 | 1674 | SAU100141 | 5236 | SAU1c0032_orf_8p | 12259 |
| S1M10000009B10 | 1674 | SAU102527 | 5693 | SAU1c0032_orf_9p | 12260 |
| S1M10000009B11 | 1675 | SAU301898 | 5904 | SAU3c1079_orf_1p | 13057 |
| S1M10000009B12 | 1676 | SAU102433 | 5668 | SAU1c0045_orf_37p | 12701 |
| S1M10000009C01 | 1677 | SAU101572 | 5484 | SAU1c0044_orf_211p | 12586 |
| S1M10000009C01 | 1677 | SAU101573 | 5485 | SAU1c0044_orf_212p | 12587 |
| S1M10000009C02 | 1678 | SAU102418 | 5664 | SAU1c0030_orf_18p | 12205 |
| S1M10000009C05 | 1679 | SAU101752 | 5522 | SAU1c0040_orf_85p | 12447 |
| S1M10000009C06 | 1680 | SAU102613 | 5715 | SAU1c0041_orf_55p | 12475 |
| S1M10000009C07 | 1681 | SAU102460 | 5678 | SAU1c0026_orf_18p | 12171 |
| S1M10000009C08 | 1682 | SAU100658 | 5303 | SAU1c0038_orf_59p | 12388 |
| S1M10000009C09 | 1683 | SAU102129 | 5604 | SAU1c0027_orf_17p | 12176 |
| S1M10000009C10 | 1684 | SAU102336 | 5646 | SAU1c0045_off_146p | 12659 |
| S1M10000009C11 | 1685 | SAU102340 | 5647 | SAU1c0045_orf_149p | 12660 |
| S1M10000009D01 | 1686 | SAU102262 | 5627 | SAU1c0032_orf_58p | 12248 |
| S1M10000009D02 | 1687 | SAU100355 | 5263 | SAU1c0023_orf_6p | 12155 |
| S1M10000009D03 | 1688 | SAU102418 | 5664 | SAU1c0030_orf_18p | 12205 |
| S1M10000009D04 | 1689 | SAU102979 | 5750 | SAU1c0043_orf_227p | 12536 |
| S1M10000009D05 | 1690 | SAU100799 | 5331 | SAU1c0045_orf_243p | 12682 |
| S1M10000009D07 | 1691 | SAU200994 | 5802 | SAU2c0428_orf_4p | 12935 |
| S1M10000009D09 | 1692 | SAU101681 | 5510 | SAU1c0044_orf_220p | 12592 |
| S1M10000009D09 | 1692 | SAU101682 | 5511 | SAU1c0044_orf_219p | 12591 |
| S1M10000009D11 | 1693 | SAU101455 | 5456 | SAU1c0045_orf_250p | 12686 |
| S1M10000009D11 | 1693 | SAU200916 | 5797 | SAU2c0373_orf_4p | 12838 |
| S1M10000009D11 | 1693 | SAU301620 | 5899 | SAU3c1478_orf_2p | 13140 |
| S1M10000009E02 | 1694 | SAU101572 | 5484 | SAU1c0044_orf_211p | 12586 |
| S1M10000009E02 | 1694 | SAU101573 | 5485 | SAU1c0044_orf_212p | 12587 |
| S1M10000009E06 | 1695 | SAU102059 | 5597 | SAU1c0034_orf_51p | 1286 |
| S1M10000009E08 | 1696 | SAU201539 | 5821 | SAU2c0431_orf_15p | 12943 |
| S1M10000009E09 | 1697 | SAU100114 | 5228 | SAU1c0043_orf_225p | 12535 |
| S1M10000009E11 | 1698 | SAU101501 | 5541 | #N/A | #N/A |
| S1M10000009E12 | 1699 | SAU101572 | 5484 | SAU1c0044_orf_211p | 12586 |
| S1M10000009F01 | 1700 | SAU101452 | 5455 | SAU1c0045_orf_247p | 12684 |
| S1M10000009F02 | 1701 | SAU101818 | 5553 | SAU1c0038_orf_20p | 12369 |
| S1M10000009F03 | 1702 | SAU101488 | 5463 | SAU1c0025_orf_18p | 12164 |
| S1M10000009F05 | 1703 | SAU101752 | 5522 | SAU1c0040_orf_85p | 12447 |
| S1M10000009F06 | 1704 | SAU101752 | 5522 | SAU1c0040_orf_85p | 12447 |
| S1M10000009F07 | 1705 | SAU102607 | 5712 | SAU1c0041_orf_51p | 12472 |
| S1M10000009F07 | 1705 | SAU102944 | 5749 | SAU1c0041_orf_47p | 12468 |
| S1M10000009F09 | 1706 | SAU202176 | 5846 | SAU2c0412_orf_3p | 12895 |
| S1M10000009F09 | 1706 | SAU302805 | 5911 | SAU3c1458_orf_1p | 13133 |
| S1M10000009F10 | 1707 | SAU102392 | 5658 | SAU1c0033_orf_40p | 12270 |
| S1M10000009F10 | 1707 | SAU201541 | 5822 | SAU2c0431_orf_14p | 12942 |
| S1M10000009G02 | 1708 | SAU101572 | 5484 | SAU1c0044_orf_211p | 12586 |
| S1M10000009G02 | 1708 | SAU101573 | 5485 | SAU1c0044_orf_212p | 12587 |
| S1M10000009G03 | 1709 | SAU301620 | 5899 | SAU3c1478_orf_2p | 13140 |
| S1M10000009G05 | 1710 | SAU101752 | 5522 | SAU1c0040_orf_85p | 12447 |
| S1M10000009G06 | 1711 | SAU102909 | 5743 | SAU1c0036_orf_16p | 12315 |
| S1M10000009G07 | 1712 | SAU200468 | 5781 | SAU2c0429_orf_19p | 12937 |
| S1M10000009G09 | 1713 | SAU102693 | 5731 | SAU1c0044_orf_58p | 12627 |
| S1M10000009G10 | 1714 | SAU100646 | 5302 | SAU1c0025_orf_5p | 12168 |
| S1M10000009G11 | 1715 | SAU100131 | 5232 | SAU1c0043_orf_156p | 12517 |
| S1M10000009H01 | 1716 | SAU201506 | 5818 | SAU2c0432_orf_18p | 12946 |
| S1M10000009H02 | 1717 | SAU102658 | 5726 | SAU1c0045_orf_121p | 12654 |
| S1M10000009H03 | 1718 | SAU201654 | 5829 | SAU2c0442_orf_12p | 12982 |
| S1M10000009H05 | 1719 | SAU100582 | 5292 | SAU1c0042_orf_21p | 12503 |
| S1M10000009H05 | 1719 | SAU102165 | 5610 | SAU1c0041_orf_25p | 12460 |
| S1M10000009H05 | 1719 | SAU201929 | 5838 | SAU2c0451_orf_19p | 13008 |
| S1M10000009H07 | 1720 | SAU102297 | 5640 | SAU1c0045_orf_41p | 12704 |
| S1M10000009H09 | 1721 | SAU200928 | 5798 | SAU2c0365_orf_5p | 12815 |
| S1M10000009H11 | 1722 | SAU101801 | 5541 | #N/A | #N/A |
| S1M10000011A02 | 1723 | SAU100414 | 5270 | SAU1c0022_orf_24p | 12148 |
| S1M10000011A03 | 1724 | SAU101271 | 5411 | SAU1c0037_orf_90p | 12366 |
| S1M10000011A04 | 1725 | SAU101791 | 5532 | SAU1c0032_orf_12p | 12216 |
| S1M10000011A06 | 1726 | SAU101574 | 5486 | SAU1c0044_orf_213p | 12588 |
| S1M10000011A06 | 1726 | SAU101575 | 5487 | SAU1c0044_orf_214p | 12589 |
| S1M10000011B01 | 1727 | SAU102881 | 5740 | SAU1c0032_orf_4p | 12242 |
| S1M10000011B02 | 1728 | SAU101541 | 5472 | SAU1c0037_orf_128p | 12344 |
| S1M10000011B03 | 1729 | SAU101849 | 5559 | SAU1c0044_orf_148p | 12567 |
| S1M10000011B04 | 1730 | SAU101574 | 5486 | SAU1c0044_orf_213p | 12588 |
| S1M10000011B04 | 1730 | SAU101575 | 5487 | SAU1c0044_orf_214p | 12589 |
| S1M10000011B05 | 1731 | SAU200934 | 5799 | SAU2c0375_orf_9p | 12842 |
| S1M10000011C01 | 1732 | SAU101447 | 5454 | SAU1c0045_orf_244p | 12683 |
| S1M10000011C05 | 1733 | SAU100432 | 5271 | SAU1c0040_orf_88p | 12450 |
| S1M10000011C05 | 1733 | SAU202756 | 5852 | SAU2c0470_orf_1p | 13027 |
| S1M10000011C06 | 1734 | SAU102350 | 5649 | SAU1c0040_orf_36p | 12433 |
| S1M10000011D01 | 1735 | SAU101293 | 5414 | SAU1c0044_orf_61p | 12631 |
| S1M10000011D02 | 1736 | SAU100414 | 5270 | SAU1c0022_orf_24p | 12148 |
| S1M10000011D04 | 1737 | SAU102280 | 5632 | SAU1c0038_orf_3p | 12378 |
| S1M10000011D06 | 1738 | SAU102942 | 5748 | SAU1c0035_orf_103p | 12296 |
| S1M10000011E02 | 1739 | SAU101966 | 5580 | SAU1c0028_orf_41p | 12186 |
| S1M10000011E03 | 1740 | SAU101632 | 5499 | SAU1c0039_orf_3p | 12407 |
| S1M10000011E04 | 1741 | SAU101572 | 5484 | SAU1c0044_orf_211p | 12586 |
| S1M10000011F01 | 1742 | SAU101365 | 5432 | SAU1c0044_orf_112p | 12556 |
| S1M10000011F03 | 1743 | SAU102350 | 5649 | SAU1c0040_orf_36p | 12433 |
| S1M10000011F04 | 1744 | SAU101155 | 5385 | SAU1c0036_orf_11p | 12310 |
| S1M10000011F06 | 1745 | SAU101481 | 5460 | SAU1c0015_orf_9p | 12130 |
| S1M10000011F06 | 1745 | SAU101482 | 5461 | SAU1c0015_orf_10p | 12123 |
| S1M10000011G01 | 1746 | SAU301465 | 5896 | SAU3c1429_orf_4p | 13121 |
| S1M10000011G03 | 1747 | SAU302626 | 5907 | SAU3c1367_orf_3p | 13105 |
| S1M10000011G04 | 1748 | SAU101271 | 5411 | SAU1c0037_orf_90p | 12366 |
| S1M10000011G05 | 1749 | SAU102350 | 5649 | SAU1c0040_orf_36p | 12433 |
| S1M10000011G06 | 1750 | SAU102298 | 5641 | SAU1c0045_orf_42p | 12705 |
| S1M10000011H01 | 1751 | SAU201558 | 5823 | SAU2c0434_orf_5p | 12954 |
| S1M10000011H03 | 1752 | SAU100432 | 5271 | SAU1c0040_orf_88p | 12450 |
| S1M10000011H03 | 1752 | SAU202756 | 5852 | SAU2c0470_orf_1p | 13027 |
| S1M10000011H04 | 1753 | SAU200934 | 5799 | SAU2c0375_orf_9p | 12842 |
| S1M10000012A02 | 1754 | SAU102533 | 5695 | #N/A | #N/A |
| S1M10000012A02 | 1754 | SAU102534 | 5696 | #N/A | #N/A |
| S1M10000012A06 | 1755 | SAU100157 | 5237 | SAU1c0040_orf_81p | 12444 |
| S1M10000012A08 | 1756 | SAU101630 | 5498 | SAU1c0039_orf_4p | 12410 |
| S1M10000012A08 | 1756 | SAU300156 | 5867 | SAU3c0609_orf_2p | 13036 |
| S1M10000012A09 | 1757 | SAU102356 | 5652 | SAU1c0040_orf_41p | 12436 |
| S1M10000012A10 | 1758 | SAU101266 | 5408 | SAU1c0042_orf_117p | 12490 |
| S1M10000012A11 | 1759 | SAU100390 | 5267 | #N/A | #N/A |
| S1M10000012A11 | 1759 | SAU200028 | 5771 | SAU2c0145_orf_1p | 12721 |
| S1M10000012B01 | 1760 | SAU100751 | 5321 | SAU1c0036_orf_59p | 12335 |
| S1M10000012B05 | 1761 | SAU101573 | 5485 | SAU1c0044_orf_212p | 12587 |
| S1M10000012B06 | 1762 | SAU102350 | 5649 | SAU1c0040_orf_36p | 12433 |
| S1M10000012B07 | 1763 | SAU101814 | 5551 | SAU1c0032_orf_32p | 12237 |
| S1M10000012B07 | 1763 | SAU101815 | 5552 | SAU1c0032_orf_33p | 12238 |
| S1M10000012B11 | 1764 | SAU102551 | 5698 | SAU1c0045_orf_206p | 12672 |
| S1M10000012C01 | 1765 | SAU101652 | 5503 | SAU1c0042_orf_123p | 12492 |
| S1M10000012C03 | 1766 | SAU100776 | 5327 | SAU1c0041_orf_72p | 12482 |
| S1M10000012C04 | 1767 | SAU100776 | 5327 | SAU1c0041_orf_72p | 12482 |
| S1M10000012C05 | 1768 | SAU201558 | 5823 | SAU2c0434_orf_5p | 12954 |
| S1M10000012C06 | 1769 | SAU101570 | 5482 | SAU1c0044_orf_209p | 12584 |
| S1M10000012C06 | 1769 | SAU101571 | 5483 | SAU1c0044_orf_210p | 12585 |
| S1M10000012C11 | 1770 | SAU100547 | 5290 | SAU1c0032_orf_3p | 12240 |
| S1M10000012C11 | 1770 | SAU102881 | 5740 | SAU1c0032_orf_4p | 12242 |
| S1M10000012C12 | 1771 | SAU101781 | 5528 | SAU1c0037_orf_43p | 12353 |
| S1M10000012D04 | 1772 | SAU201952 | 5839 | SAU2c0457_orf_10p | 13020 |
| S1M10000012D06 | 1773 | SAU101271 | 5411 | SAU1c0037_orf_90p | 12366 |
| S1M10000012D07 | 1774 | SAU200928 | 5798 | SAU2c0365_orf_5p | 12815 |
| S1M10000012D08 | 1775 | SAU101652 | 5503 | SAU1c0042_orf_123p | 12492 |
| S1M10000012D09 | 1776 | SAU101752 | 5522 | SAU1c0040_orf_85p | 12447 |
| S1M10000012D12 | 1777 | SAU102620 | 5718 | SAU1c0041_orf_62p | 12479 |
| S1M10000012D12 | 1777 | SAU102621 | 5719 | SAU1c0041_orf_63p | 12480 |
| S1M10000012D12 | 1777 | SAU202006 | 5842 | SAU2c0456_orf_20p | 13018 |
| S1M10000012E01 | 1778 | SAU100733 | 5314 | SAU1c0044_orf_254p | 12602 |
| S1M10000012E01 | 1778 | SAU100734 | 5315 | SAU1c0044_orf_255p | 12603 |
| S1M10000012E02 | 1779 | SAU102485 | 5686 | SAU1c0039_orf_95p | 12421 |
| S1M10000012E04 | 1780 | SAU201486 | 5817 | SAU2c0457_orf_34p | 13023 |
| S1M10000012E07 | 1781 | SAU100390 | 5267 | #N/A | #N/A |
| S1M10000012E07 | 1781 | SAU200028 | 5771 | SAU2c0145_orf_1p | 12721 |
| S1M10000012E08 | 1782 | SAU101189 | 5392 | SAU1c0033_orf_25p | 12264 |
| S1M10000012E12 | 1783 | SAU201810 | 5836 | SAU2c0308_orf_2p | 12769 |
| S1M10000012E12 | 1783 | SAU202174 | 5845 | SAU2c0412_orf_3p | 12895 |
| S1M10000012E12 | 1783 | SAU301148 | 5888 | #N/A | #N/A |
| S1M10000012F04 | 1784 | SAU101793 | 5534 | SAU1c0032_orf_14p | 12218 |
| S1M10000012F07 | 1785 | SAU102284 | 5635 | SAU1c0038_orf_5p | 12389 |
| S1M10000012F07 | 1785 | SAU201469 | 5816 | SAU2c0438_orf_6p | 12967 |
| S1M10000012F08 | 1786 | SAU101189 | 5392 | SAU1c0033_orf_25p | 12264 |
| S1M10000012F09 | 1787 | SAU201403 | 5815 | SAU2c0423_orf_3p | 12913 |
| S1M10000012F10 | 1788 | SAU101752 | 5522 | SAU1c0040_orf_85p | 12447 |
| S1M10000012F11 | 1789 | SAU101781 | 5528 | SAU1c0037_orf_43p | 12353 |
| S1M10000012F12 | 1790 | SAU201810 | 5836 | SAU2c0308_orf_2p | 12769 |
| S1M10000012F12 | 1790 | SAU202174 | 5845 | SAU2c0412_orf_3p | 12895 |
| S1M10000012F12 | 1790 | SAU301148 | 5888 | #N/A | #N/A |
| S1M10000012G01 | 1791 | SAU102117 | 5603 | SAU1c0027_orf_6p | 12181 |
| S1M10000012G02 | 1792 | SAU301758 | 5900 | SAU3c1508_orf_5p | 13156 |
| S1M10000012G03 | 1793 | SAU201301 | 5809 | SAU2c0416_orf_17p | 12899 |
| S1M10000012G06 | 1794 | SAU101571 | 5483 | SAU1c0044_orf_210p | 12585 |
| S1M10000012G07 | 1795 | SAU101572 | 5484 | SAU1c0044_orf_211p | 12586 |
| S1M10000012G07 | 1795 | SAU101573 | 5485 | SAU1c0044_orf_212p | 12587 |
| S1M10000012G08 | 1796 | SAU102593 | 5704 | SAU1c0041_orf_39p | 12463 |
| S1M10000012G10 | 1797 | SAU100887 | 5350 | SAU1c0018_orf_15p | 12138 |
| S1M10000012H05 | 1798 | SAU100157 | 5237 | SAU1c0040_orf_81p | 12444 |
| S1M10000012H08 | 1799 | SAU202186 | 5847 | SAU2c0222_orf_1p | 12731 |
| S1M10000012H09 | 1800 | SAU100227 | 5244 | SAU1c0043_orf_188p | 12525 |
| S1M10000012H10 | 1801 | SAU100432 | 5271 | SAU1c0040_orf_88p | 12450 |
| S1M10000012H10 | 1801 | SAU100433 | 5272 | SAU1c0040_orf_87p | 12449 |
| S1M10000012H10 | 1801 | SAU101751 | 5521 | SAU1c0040_orf_86p | 12448 |
| S1M10000012H11 | 1802 | SAU301118 | 5886 | SAU3c1305_orf_3p | 13086 |
| S1M10000013A02 | 1803 | SAU102674 | 5730 | SAU1c0024_orf_12p | 12156 |
| S1M10000013A03 | 1804 | SAU101006 | 5367 | SAU1c0028_orf_59p | 12190 |
| S1M10000013A05 | 1805 | SAU102450 | 5675 | SAU1c0045_orf_21p | 12675 |
| S1M10000013A07 | 1806 | SAU102602 | 5708 | SAU1c0032_orf_5p | 12249 |
| S1M10000013A08 | 1807 | SAU101143 | 5383 | SAU1c0042_orf_159p | 12502 |
| S1M10000013A09 | 1808 | SAU101567 | 5481 | SAU1c0022_orf_10p | 12144 |
| S1M10000013A09 | 1808 | SAU200030 | 5772 | SAU2c0282_orf_3p | 12745 |
| S1M10000013A10 | 1809 | SAU201403 | 5815 | SAU2c0423_orf_3p | 12913 |
| S1M10000013A11 | 1810 | SAU101573 | 5485 | SAU1c0044_orf_212p | 12587 |
| S1M10000013A12 | 1811 | SAU100690 | 5309 | #N/A | #N/A |
| S1M10000013B02 | 1812 | SAU100433 | 5272 | SAU1c0040_orf_87p | 12449 |
| S1M10000013B03 | 1813 | SAU201236 | 5808 | SAU2c0409_orf_10p | 12891 |
| S1M10000013B04 | 1814 | SAU200928 | 5798 | SAU2c0365_orf_5p | 12815 |
| S1M10000013B05 | 1815 | SAU100300 | 5253 | SAU1c0040_orf_90p | 12451 |
| S1M10000013B06 | 1816 | SAU100118 | 5229 | SAU1c0015_orf_13p | 12125 |
| S1M10000013B07 | 1817 | SAU202174 | 5845 | SAU2c0412_orf_3p | 12895 |
| S1M10000013B07 | 1817 | SAU301148 | 5888 | #N/A | #N/A |
| S1M10000013B09 | 1818 | SAU200006 | 5770 | SAU2c0157_orf_1p | 12723 |
| S1M10000013B11 | 1819 | SAU103042 | 5758 | #N/A | #N/A |
| S1M10000013C03 | 1820 | SAU101781 | 5528 | SAU1c0037_orf_43p | 12353 |
| S1M10000013C05 | 1821 | SAU101038 | 5372 | SAU1c0043_orf_180p | 12521 |
| S1M10000013C07 | 1822 | SAU100300 | 5253 | SAU1c0040_orf_90p | 12451 |
| S1M10000013C08 | 1823 | SAU101571 | 5483 | SAU1c0044_orf_210p | 12585 |
| S1M10000013C09 | 1824 | SAU102059 | 5597 | SAU1c0034_orf_51p | 12286 |
| S1M10000013C10 | 1825 | SAU100736 | 5316 | SAU1c0038_orf_64p | 12391 |
| S1M10000013C11 | 1826 | SAU102059 | 5597 | SAU1c0034_orf_51p | 12286 |
| S1M10000013C12 | 1827 | SAU103038 | 5757 | #N/A | #N/A |
| S1M10000013D08 | 1828 | SAU101798 | 5538 | SAU1c0032_orf_18p | 12222 |
| S1M10000013D09 | 1829 | SAU102669 | 5728 | SAU1c0024_orf_7p | 12160 |
| S1M10000013D09 | 1829 | SAU302956 | 5915 | SAU3c1513_orf_9p | 13161 |
| S1M10000013D11 | 1830 | SAU102433 | 5668 | SAU1c0045_orf_37p | 12701 |
| S1M10000013E01 | 1831 | SAU102674 | 5730 | SAU1c0024_orf_12p | 12156 |
| S1M10000013E02 | 1832 | SAU101184 | 5391 | SAU1c0035_orf_80p | 12305 |
| S1M10000013E04 | 1833 | SAU101802 | 5542 | SAU1c0032_orf_22p | 12227 |
| S1M10000013E06 | 1834 | SAU101833 | 5555 | SAU1c0038_orf_34p | 12373 |
| S1M10000013E08 | 1835 | SAU100831 | 5335 | SAU1c0038_orf_93p | 12403 |
| S1M10000013E09 | 1836 | SAU101571 | 5483 | SAU1c0044_orf_210p | 12585 |
| S1M10000013E10 | 1837 | SAU101801 | 5541 | #N/A | #N/A |
| S1M10000013F02 | 1838 | SAU101570 | 5482 | SAU1c0044_orf_209p | 12584 |
| S1M10000013F03 | 1839 | SAU101907 | 5574 | SAU1c0040_orf_79p | 12442 |
| S1M10000013F06 | 1840 | SAU103038 | 5757 | #N/A | #N/A |
| S1M10000013F07 | 1841 | SAU101545 | 5474 | SAU1c0037_orf_132p | 12348 |
| S1M10000013F08 | 1842 | SAU100961 | 5360 | SAU1c0044_orf_83p | 12638 |
| S1M10000013F09 | 1843 | SAU101398 | 5442 | SAU1c0036_orf_33p | 12324 |
| S1M10000013F12 | 1844 | SAU102437 | 5670 | SAU1c0045_orf_33p | 12695 |
| S1M10000013G01 | 1845 | SAU100521 | 5283 | SAU1c0044_orf_250p | 12600 |
| S1M10000013G04 | 1846 | SAU101592 | 5490 | SAU1c0039_orf_37p | 12406 |
| S1M10000013G05 | 1847 | SAU102241 | 5617 | SAU1c0043_orf_25p | 12539 |
| S1M10000013G05 | 1847 | SAU102242 | 5618 | SAU1c0043_orf_26p | 12540 |
| S1M10000013G06 | 1848 | SAU102380 | 5654 | SAU1c0033_orf_29p | 12265 |
| S1M10000013G07 | 1849 | SAU101573 | 5485 | SAU1c0044_orf_212p | 12587 |
| S1M10000013G10 | 1850 | SAU201539 | 5821 | SAU2c0431_orf_15p | 12943 |
| S1M10000013G11 | 1851 | SAU101890 | 5570 | SAU1c0034_orf_29p | 12280 |
| S1M10000013G12 | 1852 | SAU100843 | 5339 | SAU1c0036_orf_40p | 12328 |
| S1M10000013H03 | 1853 | SAU100690 | 5309 | #N/A | #N/A |
| S1M10000013H04 | 1854 | SAU102450 | 5675 | SAU1c0045_orf_21p | 12675 |
| S1M10000013H05 | 1855 | SAU200914 | 5796 | SAU2c0373_orf_2p | 12837 |
| S1M10000013H07 | 1856 | SAU100414 | 5270 | SAU1c0022_orf_24p | 12148 |
| S1M10000013H09 | 1857 | SAU100444 | 5275 | SAU1c0038_orf_67p | 12392 |
| S1M10000013H09 | 1857 | SAU200721 | 5791 | SAU2c0339_orf_5p | 12797 |
| S1M10000013H10 | 1858 | SAU102059 | 5597 | SAU1c0034_orf_51p | 12286 |
| S1M10000013H11 | 1859 | SAU100690 | 5309 | #N/A | #N/A |
| S1M10000014A02 | 1860 | SAU200564 | 5784 | SAU2c0324_orf_6p | 12780 |
| S1M10000014A03 | 1861 | SAU101310 | 5418 | SAU1c0044_orf_125p | 12562 |
| S1M10000014A05 | 1862 | SAU101991 | 5582 | SAU1c0040_orf_94p | 12454 |
| S1M10000014A07 | 1863 | SAU101526 | 5470 | SAU1c0027_orf_32p | 12179 |
| S1M10000014A08 | 1864 | SAU103038 | 5757 | #N/A | #N/A |
| S1M10000014A11 | 1865 | SAU100866 | 5344 | SAU1c0044_orf_100p | 12553 |
| S1M10000014A12 | 1866 | SAU201571 | 5824 | SAU2c0447_orf_17p | 12997 |
| S1M10000014B01 | 1867 | SAU100547 | 5290 | SAU1c0032_orf_3p | 12240 |
| S1M10000014B02 | 1868 | SAU100432 | 5271 | SAU1c0040_orf_88p | 12450 |
| S1M10000014B02 | 1868 | SAU100433 | 5272 | SAU1c0040_orf_87p | 12449 |
| S1M10000014B03 | 1869 | SAU100414 | 5270 | SAU1c0022_orf_24p | 12148 |
| S1M10000014B04 | 1870 | SAU100778 | 5328 | SAU1c0043_orf_140p | 12514 |
| S1M10000014B05 | 1871 | SA1310476 | 5682 | SAU1c0026_orf_33p | 12175 |
| S1M10000014B06 | 1872 | SAU101199 | 5395 | SAU1c0035_orf_62p | 12302 |
| S1M10000014B07 | 1873 | SAU101756 | 5524 | SAU1c0040_orf_82p | 12445 |
| S1M10000014B08 | 1874 | SAU101752 | 5522 | SAU1c0040_orf_85p | 12447 |
| S1M10000014B10 | 1875 | SAU200006 | 5770 | SAU2c0157_orf_1p | 12723 |
| S1M10000014B11 | 1876 | SAU102534 | 5696 | #N/A | #N/A |
| S1M10000014B12 | 1877 | SAU102534 | 5696 | #N/A | #N/A |
| S1M10000014C01 | 1878 | SAU101575 | 5487 | SAU1c0044_orf_214p | 12589 |
| S1M10000014c05 | 1879 | SAU102602 | 5708 | SAU1c0032_orf_5p | 12249 |
| S1M10000014C06 | 1880 | SAU100305 | 5256 | SAU1c0038_orf_77p | 12397 |
| S1M10000014C07 | 1881 | SAU101801 | 5541 | #N/A | #N/A |
| S1M10000014C09 | 1882 | SAU100547 | 5290 | SAU1c0032_orf_3p | 12240 |
| S1M10000014C09 | 1882 | SAU102881 | 5740 | SAU1c0032_orf_4p | 12242 |
| S1M10000014C10 | 1883 | SAU302901 | 5912 | SAU3c1497_orf_8p | 13146 |
| S1M10000014C11 | 1884 | SAU100514 | 5281 | SAU1c0044_orf_57p | 12626 |
| S1M10000014C12 | 1885 | SAU101814 | 5551 | SAU1c0032_orf_32pf | 12237 |
| S1M10000014C12 | 1885 | SAU101815 | 5552 | SAU1c0032_orf_33p | 12238 |
| S1M10000014D03 | 1886 | SAU100885 | 5348 | SAU1c0038_orf_38p | 12376 |
| S1M10000014D06 | 1887 | SAU100305 | 5256 | SAU1c0038_orf_77p | 12397 |
| S1M10000014D08 | 1888 | SAU101752 | 5522 | SAU1c0040_orf_85p | 12447 |
| S1M10000014D09 | 1889 | SAU100808 | 5332 | SAU1c0037_orf_12p | 12345 |
| S1M10000014D10 | 1890 | SAU102292 | 5638 | SAU1c0038_orf_10p | 12368 |
| S1M10000014E01 | 1891 | SAU101793 | 5534 | SAU1c0032_orf_14p | 12218 |
| S1M10000014E01 | 1891 | SAU101794 | 5535 | #N/A | #N/A |
| S1M10000014E04 | 1892 | SAU100964 | 5363 | SAU1c0044_orf_86p | 12641 |
| S1M10000014E05 | 1893 | SAU101565 | 5480 | SAU1c0022_orf_8p | 12151 |
| S1M10000014E07 | 1894 | SAU100658 | 5303 | SAU1c0038_orf_59p | 12388 |
| S1M10000014E07 | 1894 | SAU100659 | 5304 | SAU1c0038_orf_60p | 12390 |
| S1M10000014E08 | 1895 | SAU202176 | 5846 | SAU2c0412_orf_3p | 12895 |
| S1M10000014E09 | 1896 | SAU102059 | 5597 | SAU1c0034_orf_51p | 12286 |
| S1M10000014E09 | 1896 | SAU300269 | 5869 | #N/A | #N/A |
| S1M10000014E10 | 1897 | SAU102453 | 5677 | SAU1c0045_orf_19p | 12669 |
| S1M10000014E12 | 1898 | SAU102284 | 5635 | SAU1c0038_orf_5p | 12389 |
| S1M10000014E12 | 1898 | SAU201469 | 5816 | SAU2c0438_orf_6p | 12967 |
| S1M10000014F02 | 1899 | SAU100128 | 5231 | #N/A | #N/A |
| S1M10000014F02 | 1899 | SAU101549 | 5476 | SAU1c0043_orf_64p | 12549 |
| S1M10000014F02 | 1899 | SAU101576 | 5488 | SAU1c0044_orf_105p | 12554 |
| S1M10000014F03 | 1900 | SAU102200 | 5611 | SAU1c0045_orf_168p | 12665 |
| S1M10000014F03 | 1900 | SAU102201 | 5612 | SAU1c0045_orf_169p | 12666 |
| S1M10000014F04 | 1901 | SAU102449 | 5674 | SAU1c0045_orf_22p | 12677 |
| S1M10000014F05 | 1902 | SAU200914 | 5796 | SAU2c0373_orf_2p | 12837 |
| S1M10000014F08 | 1903 | SAU102433 | 5668 | SAU1c0045_orf_37p | 12701 |
| S1M10000014F09 | 1904 | SAU102059 | 5597 | SAU1c0034_orf_51p | 12286 |
| S1M10000014F09 | 1904 | SAU300269 | 5869 | #N/A | #N/A |
| S1M10000014F10 | 1905 | SAU100887 | 5350 | SAU1c0018_orf_15p | 12138 |
| S1M10000014G02 | 1906 | SAU102054 | 5596 | SAU1c0039_orf_74p | 12417 |
| S1M10000014G04 | 1907 | SAU101242 | 5404 | SAU1c0044_orf_18p | 12578 |
| S1M10000014G06 | 1908 | SAU100275 | 5252 | SAU1c0036_orf_15p | 12314 |
| S1M10000014G07 | 1909 | SAU201620 | 5827 | #N/A | #N/A |
| S1M10000014G08 | 1910 | SAU100157 | 5237 | SAU1c0040_orf_81p | 12444 |
| S1M10000014G12 | 1911 | SAU102602 | 5708 | SAU1c0032_orf_5p | 12249 |
| S1M10000014H02 | 1912 | SAU100242 | 5246 | SAU1c0036_orf_5p | 12336 |
| S1M10000014H03 | 1913 | SAU102264 | 5628 | SAU1c0032_orf_60p | 12250 |
| S1M10000014H04 | 1914 | SAU100275 | 5252 | SAU1c0036_orf_15p | 12314 |
| S1M10000014H05 | 1915 | SAU102116 | 5602 | SAU1c0027_orf_5p | 12180 |
| S1M10000014H06 | 1916 | SAU100275 | 5252 | SAU1c0036_orf_15p | 12314 |
| S1M10000014H07 | 1917 | SAU103038 | 5757 | #N/A | #N/A |
| S1M10000014H08 | 1918 | SAU100157 | 5237 | SAU1c0040_orf_81p | 12444 |
| S1M10000014H11 | 1919 | SAU102534 | 5696 | #N/A | #N/A |
| S1M10000015A02 | 1920 | SAU100865 | 5343 | SAU1c0044_orf_99p | 12648 |
| S1M10000015A03 | 1921 | SAU102388 | 5655 | SAU1c0033_orf_35p | 12267 |
| S1M10000015A05 | 1922 | SAU101815 | 5552 | SAU1c0032_orf_33p | 12238 |
| S1M10000015A06 | 1923 | SAU101857 | 5560 | SAU1c0044_orf_156p | 12569 |
| S1M10000015A09 | 1924 | SAU100414 | 5270 | SAU1c0022_orf_24p | 12148 |
| S1M10000015A10 | 1925 | SAU103038 | 5757 | #N/A | #N/A |
| S1M10000015A11 | 1926 | SAU101791 | 5532 | SAU1c0032_orf_12p | 12216 |
| S1M10000015A12 | 1927 | SAU100158 | 5238 | SAU1c0040_orf_80p | 12443 |
| S1M10000015B02 | 1928 | SAU102340 | 5647 | SAU1c0045_orf_149p | 12660 |
| S1M10000015B05 | 1929 | SAU103038 | 5757 | #N/A | #N/A |
| S1M10000015B08 | 1930 | SAU101791 | 5532 | SAU1c0032_orf_12p | 12216 |
| S1M10000015B08 | 1930 | SAU101792 | 5533 | SAU1c0032_orf_13p | 12217 |
| S1M10000015B09 | 1931 | SAU102585 | 5703 | SAU1c0044_orf_289p | 12611 |
| S1M10000015B09 | 1931 | SAU201773 | 5834 | SAU2c0446_orf_4p | 12996 |
| S1M10000015B09 | 1931 | SAU302685 | 5908 | SAU3c1403_orf_1p | 13113 |
| S1M10000015B10 | 1932 | SAU102308 | 5642 | SAU1c0045_orf_50p | 12706 |
| S1M10000015C01 | 1933 | SAU100158 | 5238 | SAU1c0040_orf_80p | 12443 |
| S1M10000015C02 | 1934 | SAU102340 | 5647 | SAU1c0045_orf_149p | 12660 |
| S1M10000015C03 | 1935 | SAU102390 | 5657 | SAU1c0033_orf_38p | 12269 |
| S1M10000015C03 | 1935 | SAU201333 | 5810 | SAU2c0418_orf_8p | 12905 |
| S1M10000015C05 | 1936 | SAU100690 | 5309 | #N/A | #N/A |
| S1M10000015C06 | 1937 | SAU101815 | 5552 | SAU1c0032_orf_33p | 12238 |
| S1M10000015C08 | 1938 | SAU100133 | 5233 | SAU1c0044_orf_170p | 12574 |
| S1M10000015C08 | 1938 | SAU100323 | 5261 | SAU1c0044_orf_171p | 12575 |
| S1M10000015C10 | 1939 | SAU100414 | 5270 | SAU1c0022_orf_24p | 12148 |
| S1M10000015C12 | 1940 | SAU100305 | 5256 | SAU1c0038_orf_77p | 12397 |
| S1M10000015D02 | 1941 | SAU100794 | 5330 | SAU1c0028_orf_53p | 12189 |
| S1M10000015D03 | 1942 | SAU102032 | 5591 | SAU1c0029_orf_47p | 12198 |
| S1M10000015D04 | 1943 | SAU100131 | 5232 | SAU1c0043_orf_156p | 12517 |
| S1M10000015D05 | 1944 | SAU100793 | 5329 | SAU1c0028_orf_52p | 12188 |
| S1M10000015D06 | 1945 | SAU100736 | 5316 | SAU1c0038_orf_64p | 12391 |
| S1M10000015D12 | 1946 | SAU101814 | 5551 | SAU1c0032_orf_32p | 12237 |
| S1M10000015E02 | 1947 | SAU102390 | 5657 | SAU1c0033_orf_38p | 12269 |
| S1M10000015E02 | 1947 | SAU201333 | 5810 | SAU2c0418_orf_8p | 12905 |
| S1M10000015E03 | 1948 | SAU200468 | 5781 | SAU2c0429_orf_19p | 12937 |
| S1M10000015E06 | 1949 | SAU101320 | 5420 | SAU1c0015_orf_16p | 12128 |
| S1M10000015E07 | 1950 | SAU101545 | 5474 | SAU1c0037_orf_132p | 12348 |
| S1M10000015E09 | 1951 | SAU102433 | 5668 | SAU1c0045_orf_37p | 12701 |
| S1M10000015E10 | 1952 | SAU100114 | 5228 | SAU1c0043_orf_225p | 12535 |
| S1M10000015E11 | 1953 | SAU102286 | 5636 | SAU1c0038_orf_6p | 12393 |
| S1M10000015E11 | 1953 | SAU102287 | 5637 | SAU1c0038_orf_7p | 12398 |
| S1M10000015E12 | 1954 | SAU102352 | 5650 | SAU1c0040_orf_38p | 12434 |
| S1M10000015F01 | 1955 | SAU100123 | 5230 | SAU1c0043_orf_189p | 12526 |
| S1M10000015F01 | 1955 | SAU102001 | 5586 | SAU1c0040_orf_102p | 12424 |
| S1M10000015F01 | 1955 | SAU103159 | 5762 | SAU1c0045_orf_204p | 12670 |
| S1M10000015F01 | 1955 | SAU201827 | 5837 | SAU2c0449_orf_21p | 13002 |
| S1M10000015F02 | 1956 | SAU101561 | 5479 | SAU1c0022_orf_4p | 12149 |
| S1M10000015F03 | 1957 | SAU201403 | 5815 | SAU2c0423_orf_3p | 12913 |
| S1M10000015F04 | 1958 | SAU201403 | 5815 | SAU2c0423_orf_3p | 12913 |
| S1M10000015F06 | 1959 | SAU201385 | 5814 | #N/A | #N/A |
| S1M10000015F07 | 1960 | SAU101752 | 5522 | SAU1c0040_orf_85p | 12447 |
| S1M10000015F08 | 1961 | SAU102102 | 5600 | SAU1c0045_orf_340p | 12696 |
| S1M10000015F09 | 1962 | SAU101800 | 5540 | SAU1c0032_orf_20p | 12225 |
| S1M10000015F09 | 1962 | SAU101801 | 5541 | #N/A | #N/A |
| S1M10000015F10 | 1963 | SAU100114 | 5228 | SAU1c0043_orf_225p | 12535 |
| S1M10000015G01 | 1964 | SAU102481 | 5685 | SAU1c0039_orf_99p | 12422 |
| S1M10000015G02 | 1965 | SAU200058 | 5773 | SAU2c0134_orf_1p | 12719 |
| S1M10000015G02 | 1965 | SAU200059 | 5774 | SAU2c0134_orf_3p | 12720 |
| S1M10000015G03 | 1966 | SAU101070 | 5376 | SAU1c0034_orf_60p | 12291 |
| S1M10000015G04 | 1967 | SAU101242 | 5404 | SAU1c0044_orf_18p | 12578 |
| S1M10000015G05 | 1968 | SAU101573 | 5485 | SAU1c0044_orf_212p | 12587 |
| S1M10000015G06 | 1969 | SAU101156 | 5386 | SAU1c0036_orf_12p | 12311 |
| S1M10000015G07 | 1970 | SAU100158 | 5238 | SAU1c0040_orf_80p | 12443 |
| S1M10000015G08 | 1971 | SAU101814 | 5551 | SAU1c0032_orf_32p | 12237 |
| S1M10000015G09 | 1972 | SAU102143 | 5607 | SAU1c0041_orf_14p | 12458 |
| S1M10000015G09 | 1972 | SAU102144 | 5608 | SAU1c0041_orf_15p | 12459 |
| S1M10000015G10 | 1973 | SAU101752 | 5522 | SAU1c0040_orf_85p | 12447 |
| S1M10000015G11 | 1974 | SAU100275 | 5252 | SAU1c0036_orf_15p | 12314 |
| S1M10000015H04 | 1975 | SAU101801 | 5541 | #N/A | #N/A |
| S1M10000015H04 | 1975 | SAU101802 | 5542 | SAU1c0032_orf_22p | 12227 |
| S1M10000015H06 | 1976 | SAU201385 | 5814 | #N/A | #N/A |
| S1M10000016A03 | 1977 | SAU101803 | 5543 | SAU1c0032_orf_23p | 1228 |
| S1M10000016A03 | 1977 | SAU101804 | 5544 | #N/A | #N/A |
| S1M10000016A04 | 1978 | SAU100432 | 5271 | SAU1c0040_orf_88p | 12450 |
| S1M10000016A04 | 1978 | SAU100433 | 5272 | SAU1c0040_orf_87p | 12449 |
| S1M10000016A06 | 1979 | SAU200928 | 5798 | SAU2c0365_orf_5p | 12815 |
| S1M10000016A07 | 1980 | SAU100932 | 5356 | SAU1c0044_orf_308p | 12615 |
| S1M10000016A09 | 1981 | SAU101067 | 5375 | SAU1c0034_orf_58p | 12290 |
| S1M10000016A09 | 1981 | SAU300732 | 5877 | SAU3c1116_orf_1p | 13061 |
| S1M10000016A10 | 1982 | SAU101571 | 5483 | SAU1c0044_orf_210p | 12585 |
| S1M10000016A12 | 1983 | SAU100522 | 5284 | SAU1c0044_orf_249p | 12599 |
| S1M10000016B02 | 1984 | SAU102449 | 5674 | SAU1c0045_orf_22p | 12677 |
| S1M10000016B05 | 1985 | SAU101320 | 5420 | SAU1c0015_orf_16p | 12128 |
| S1M10000016B06 | 1986 | SAU100432 | 5271 | SAU1c0040_orf_88p | 12450 |
| S1M10000016B06 | 1986 | SAU100433 | 5272 | SAU1c0040_orf_87p | 12449 |
| S1M10000016B07 | 1987 | SAU103077 | 5759 | SAU1c0039_orf_44p | 12408 |
| S1M10000016B08 | 1988 | SAU101491 | 5464 | SAU1c0025_orf_20p | 12165 |
| S1M10000016B09 | 1989 | SAU301465 | 5896 | SAU3c1429_orf_4p | 13121 |
| S1M10000016B10 | 1990 | SAU101006 | 5367 | SAU1c0028_orf_59p | 12190 |
| S1M10000016B11 | 1991 | SAU101242 | 5404 | SAU1c0044_orf_18p | 12578 |
| S1M10000016B12 | 1992 | SAU101794 | 5535 | #N/A | #N/A |
| S1M10000016B12 | 1992 | SAU101795 | 5536 | SAU1c0032_orf_15p | 12219 |
| S1M10000016C01 | 1993 | SAU100845 | 5340 | SAU1c0036_orf_41p | 12329 |
| S1M10000016C02 | 1994 | SAU102049 | 5595 | SAU1c0039_orf_68p | 12416 |
| S1M10000016C04 | 1995 | SAU100921 | 5355 | SAU1c0038_orf_76p | 12396 |
| S1M10000016C05 | 1996 | SAU101777 | 5527 | SAU1c0037_orf_39p | 12352 |
| S1M10000016C06 | 1997 | SAU201810 | 5836 | SAU2c0308_orf_2p | 12769 |
| S1M10000016C06 | 1997 | SAU202174 | 5845 | SAU2c0412_orf_3p | 12895 |
| S1M10000016C06 | 1997 | SAU301148 | 5888 | #N/A | #N/A |
| S1M10000016C08 | 1998 | SAU101491 | 5464 | SAU1c0025_orf_20p | 12165 |
| S1M10000016C09 | 1999 | SAU102233 | 5616 | SAU1c0043_orf_20p | 12531 |
| S1M10000016C10 | 2000 | SAU201513 | 5820 | SAU2c0432_orf_10p | 12944 |
| S1M10000016C10 | 2000 | SAU203196 | 5861 | SAU2c0432_orf_11p | 12945 |
| S1M10000016C11 | 2001 | SAU101573 | 5485 | SAU1c0044_orf_212p | 12587 |
| S1M10000016C12 | 2002 | SAU101752 | 5522 | SAU1c0040_orf_85p | 12447 |
| S1M10000016D01 | 2003 | SAU102355 | 5651 | SAU1c0040_orf_40p | 12435 |
| S1M10000016D02 | 2004 | SAU200242 | 5777 | SAU2c0250_orf_2p | 12734 |
| S1M10000016D04 | 2005 | SAU100921 | 5355 | SAU1c0038_orf_76p | 12396 |
| S1M10000016D05 | 2006 | SAU100770 | 5324 | #N/A | #N/A |
| S1M10000016D06 | 2007 | SAU100952 | 5358 | SAU1c0043_orf_182p | 12523 |
| S1M10000016D08 | 2008 | SAU101070 | 5376 | SAU1c0034_orf_60p | 12291 |
| S1M10000016D09 | 2009 | SAU101868 | 5565 | SAU1c0036_orf_23p | 12320 |
| S1M10000016D10 | 2010 | SAU201513 | 5820 | SAU2c0432_orf_10p | 12944 |
| S1M10000016D10 | 2010 | SAU203196 | 5861 | SAU2c0432_orf_11p | 12945 |
| S1M10000016D11 | 2011 | SAU101573 | 5485 | SAU1c0044_orf_212p | 12587 |
| S1M10000016E04 | 2012 | SAU101371 | 5435 | SAU1c0033_orf_7p | 12275 |
| S1M10000016E05 | 2013 | SAU101320 | 5420 | SAU1c0015_orf_16p | 12128 |
| S1M10000016E06 | 2014 | SAU102639 | 5724 | #N/A | #N/A |
| S1M10000016E07 | 2015 | SAU102636 | 5722 | SAU1c0045_orf_101p | 12650 |
| S1MT0000016E07 | 2015 | SAU102637 | 5723 | SAU1c0045_orf_102p | 12651 |
| S1M10000016E08 | 2016 | SAU200928 | 5798 | SAU2c0365_orf_5p | 12815 |
| S1M10000016E09 | 2017 | SAU102527 | 5693 | SAU1c0032_orf_9p | 12260 |
| S1M10000016E10 | 2018 | SAU102983 | 5751 | SAU1c0045_orf_224p | 12676 |
| S1M10000016E11 | 2019 | SAU102281 | 5633 | SAU1c0038_orf_4p | 12384 |
| S1M10000016E12 | 2020 | SAU201571 | 5824 | SAU2c0447_orf_17p | 12997 |
| S1M10000016F02 | 2021 | SAU102113 | 5601 | SAU1c0027_orf_2p | 12178 |
| S1M10000016F02 | 2021 | SAU301223 | 5889 | SAU3c1345_orf_3p | 13090 |
| S1M10000016F03 | 2022 | SAU101864 | 5562 | SAU1c0044_orf_163p | 12572 |
| S1M10000016F05 | 2023 | SAU201168 | 5804 | SAU2c0407_orf_8p | 12889 |
| S1M10000016F06 | 2024 | SAU102407 | 5662 | #N/A | #N/A |
| S1M10000016F08 | 2025 | SAU101491 | 5464 | SAU1c0025_orf_20p | 12165 |
| S1M10000016F09 | 2026 | SAU102527 | 5693 | SAU1c0032_orf_9p | 12260 |
| S1M10000016F11 | 2027 | SAU102113 | 5601 | SAU1c0027_orf_2p | 12178 |
| S1M10000016F11 | 2027 | SAU301223 | 5889 | SAU3c1345_orf_3p | 13090 |
| S1M10000016G01 | 2028 | SAU102434 | 5669 | SAU1c0045_orf_36p | 12700 |
| S1M10000016G03 | 2029 | SAU101300 | 5415 | SAU1c0044_orf_113p | 12557 |
| S1M10000016G03 | 2029 | SAU101365 | 5432 | SAU1c0044_orf_112p | 12556 |
| S1M10000016G04 | 2030 | SAU102450 | 5675 | SAU1c0045_orf_21p | 12675 |
| S1M10000016G05 | 2031 | SAU102292 | 5638 | SAU1c0038_orf_10p | 12368 |
| S1M10000016H03 | 2032 | SAU101571 | 5483 | SAU1c0044_orf_210p | 12585 |
| S1M10000016H04 | 2033 | SAU101545 | 5474 | SAU1c0037_orf_132p | 12348 |
| S1M10000016H08 | 2034 | SAU101067 | 5375 | SAU1c0034_orf_58p | 12290 |
| S1M10000016H08 | 2034 | SAU300732 | 5877 | SAU3c1116_orf_1p | 13061 |
| S1M10000016H10 | 2035 | SAU101756 | 5524 | SAU1c0040_orf_82p | 12445 |
| S1M10000017A02 | 2036 | SAU101866 | 5564 | SAU1c0036_orf_21p | 12319 |
| S1M10000017A03 | 2037 | SAU101545 | 5474 | SAU1c0037_orf_132p | 12348 |
| S1M10000017A03 | 2037 | SAU101546 | 5475 | SAU1c0037_orf_133p | 12349 |
| S1M10000017A04 | 2038 | SAU102292 | 5638 | SAU1c0038_orf_10p | 12368 |
| S1M10000017A08 | 2039 | SAU102117 | 5603 | SAU1c0027_orf_6p | 12181 |
| S1M10000017A11 | 2040 | SAU102437 | 5670 | SAU1c0045_orf_33p | 12695 |
| S1M10000017A12 | 2041 | SAU301357 | 5893 | SAU3c1394_orf_2p | 13111 |
| S1M10000017B02 | 2042 | SAU102242 | 5618 | SAU1c0043_orf_26p | 12540 |
| S1M10000017B05 | 2043 | SAU302513 | 5906 | SAU3c1298_orf_1p | 13085 |
| S1M10000017B07 | 2044 | SAU101806 | 5546 | SAU1c0032_orf_25p | 12230 |
| S1M10000017B08 | 2045 | SAU101546 | 5475 | SAU1c0037_orf_133p | 12349 |
| S1M10000017B09 | 2046 | SAU200928 | 5798 | SAU2c0365_orf_5p | 12815 |
| S1M10000017B10 | 2047 | SAU101754 | 5523 | SAU1c0040_orf_84p | 12446 |
| S1M10000017B11 | 2048 | SAU101754 | 5523 | SAU1c0040_orf_84p | 12446 |
| S1M10000017B12 | 2049 | SAU201375 | 5811 | SAU2c0426_orf_4p | 12926 |
| S1M10000017C01 | 2050 | SAU101224 | 5397 | SAU1c0044_orf_98p | 12647 |
| S1M10000017C03 | 2051 | SAU101910 | 5576 | SAU1c0040_orf_76p | 12440 |
| S1M10000017C05 | 2052 | SAU200657 | 5789 | #N/A | #N/A |
| S1M10000017C08 | 2053 | SAU101890 | 5570 | SAU1c0034_orf_29p | 12280 |
| S1M10000017C09 | 2054 | SAU101398 | 5442 | SAU1c0036_orf_33p | 12324 |
| S1M10000017C10 | 2055 | SAU102614 | 5716 | SAU1c0041_orf_56p | 12476 |
| S1M10000017C10 | 2055 | SAU102615 | 5717 | SAU1c0041_orf_57p | 12477 |
| S1M10000017C11 | 2056 | SAU101799 | 5539 | SAU1c0032_orf_19p | 12223 |
| S1M10000017C11 | 2056 | SAU101800 | 5540 | SAU1c0032_orf_20p | 12225 |
| S1M10000017C12 | 2057 | SAU101782 | 5529 | SAU1c0037_orf_44p | 12354 |
| S1M10000017C12 | 2057 | SAU200994 | 5802 | SAU2c0428_orf_4p | 12935 |
| S1M10000017D03 | 2058 | SAU101752 | 5522 | SAU1c0040_orf_85p | 12447 |
| S1M10000017D09 | 2059 | SAU101799 | 5539 | SAU1c0032_orf_19p | 12223 |
| S1M10000017D09 | 2059 | SAU101800 | 5540 | SAU1c0032_orf_20p | 12225 |
| S1M10000017D10 | 2060 | SAU100633 | 5301 | SAU1c0043_orf_147p | 12515 |
| S1M10000017E04 | 2061 | SAU101801 | 5541 | #N/A | #N/A |
| S1M10000017E05 | 2062 | SAU102334 | 5645 | SAU1c0045_orf_144p | 12658 |
| S1M10000017E08 | 2063 | SAU101198 | 5394 | SAU1c0035_orf_61p | 12301 |
| S1M10000017E11 | 2064 | SAU102883 | 5741 | SAU1c0045_orf_38p | 12702 |
| S1M10000017F01 | 2065 | SAU100157 | 5237 | SAU1c0040_orf_81p | 12444 |
| S1M10000017F04 | 2066 | SAU100140 | 5235 | SAU1c0032_orf_7p | 12258 |
| S1M10000017F04 | 2066 | SAU100141 | 5236 | SAU1c0032_orf_8p | 12259 |
| S1M10000017F05 | 2067 | SAU102541 | 5697 | SAU1c0045_orf_195p | 12668 |
| S1M10000017F06 | 2068 | SAU102356 | 5652 | SAU1c0040_orf_41p | 12436 |
| S1M10000017F11 | 2069 | SAU101463 | 5458 | SAU1c0045_orf_232p | 12679 |
| S1M10000017G02 | 2070 | SAU102433 | 5668 | SAU1c0045_orf_37p | 12701 |
| S1M10000017G05 | 2071 | SAU102259 | 5624 | SAU1c0032_orf_55p | 12245 |
| S1M10000017G06 | 2072 | SAU200565 | 5785 | SAU2c0324_orf_7p | 12781 |
| S1M10000018A03 | 2073 | SAU100139 | 5234 | SAU1c0032_orf_6p | 12255 |
| S1M10000018A03 | 2073 | SAU102602 | 5708 | SAU1c0032_orf_5p | 12249 |
| S1M10000018A04 | 2074 | SAU102142 | 5606 | SAU1c0041_orf_13p | 12457 |
| S1M10000018A05 | 2075 | SAU100886 | 5349 | SAU1c0018_orf_16p | 12139 |
| S1M10000018A05 | 2075 | SAU100887 | 5350 | SAU1c0018_orf_15p | 12138 |
| S1M10000018A06 | 2076 | SAU100970 | 5365 | SAU1c0043_orf_197p | 12529 |
| S1M10000018A08 | 2077 | SAU100139 | 5234 | SAU1c0032_orf_6p | 12255 |
| S1M10000018A08 | 2077 | SAU102602 | 5708 | SAU1c0032_orf_5p | 12249 |
| S1M10000018A09 | 2078 | SAU102142 | 5606 | SAU1c0041_orf_13p | 12457 |
| S1M10000018A10 | 2079 | SAU100866 | 5344 | SAU1c0044_orf_100p | 12553 |
| S1M10000018A11 | 2080 | SAU100139 | 5234 | SAU1c0032_orf_6p | 12255 |
| S1M10000018A11 | 2080 | SAU102602 | 5708 | SAU1c0032_orf_5p | 12249 |
| S1M10000018B02 | 2081 | SAU100886 | 5349 | SAU1c0018_orf_16p | 12139 |
| S1M10000018B02 | 2081 | SAU100887 | 5350 | SAU1c0018_orf_15p | 12138 |
| S1M10000018B03 | 2082 | SAU101839 | 5556 | SAU1c0042_orf_12p | 12495 |
| S1M10000018B05 | 2083 | SAU100300 | 5253 | SAU1c0040_orf_90p | 12451 |
| S1M10000018B09 | 2084 | SAU100836 | 5336 | SAU1c0031_orf_13p | 12212 |
| S1M10000018B09 | 2084 | SAU202731 | 5850 | #N/A | #N/A |
| S1M10000018B10 | 2085 | SAU100401 | 5268 | SAU1c0044_orf_174p | 12576 |
| S1M10000018B10 | 2085 | SAU300335 | 5870 | #N/A | #N/A |
| S1M10000018B11 | 2086 | SAU100658 | 5303 | SAU1c0038_orf_59p | 12388 |
| S1M10000018C01 | 2087 | SAU101752 | 5522 | SAU1c0040_orf_85p | 12447 |
| S1M10000018C02 | 2088 | SAU102447 | 5672 | SAU1c0045_orf_24p | 12685 |
| S1M10000018C03 | 2089 | SAU100778 | 5328 | SAU1c0043_orf_140p | 12514 |
| S1M10000018C04 | 2090 | SAU100141 | 5236 | SAU1c0032_orf_8p | 12259 |
| S1M10000018C05 | 2091 | SAU103038 | 5757 | #N/A | #N/A |
| S1M10000018C06 | 2092 | SAU100684 | 5306 | SAU1c0044_orf_68p | 12632 |
| S1M10000018C08 | 2093 | SAU102256 | 5622 | SAU1c0032_orf_52p | 12243 |
| S1M10000018C08 | 2093 | SAU102257 | 5623 | SAU1c0032_orf_53p | 12244 |
| S1M10000018C09 | 2094 | SAU101065 | 5374 | SAU1c0034_orf_56p | 12289 |
| S1M10000018C09 | 2094 | SAU102068 | 5599 | SAU1c0034_orf_55p | 12288 |
| S1M10000018C10 | 2095 | SAU100112 | 5227 | SAU1c0044_orf_70p | 12634 |
| S1M10000018C11 | 2096 | SAU102663 | 5727 | SAU1c0024_orf_2p | 12158 |
| S1M10000018C12 | 2097 | SAU101948 | 5579 | SAU1c0045_orf_69p | 12709 |
| S1M10000018D01 | 2098 | SAU101452 | 5455 | SAU1c0045_orf_247p | 12684 |
| S1M10000018D02 | 2099 | SAU102284 | 5635 | SAU1c0038_orf_5p | 12389 |
| S1M10000018D02 | 2099 | SAU201469 | 5816 | SAU2c0438_orf_6p | 12967 |
| S1M10000018D03 | 2100 | SAU101793 | 5534 | SAU1c0032_orf_14p | 12218 |
| S1M10000018D04 | 2101 | SAU101798 | 5538 | SAU1c0032_orf_18p | 12222 |
| S1M10000018D09 | 2102 | SAU101067 | 5375 | SAU1c0034_orf_58p | 12290 |
| S1M10000018D10 | 2103 | SAU301898 | 5904 | SAU3c1079_orf_1p | 13057 |
| S1M10000018D11 | 2104 | SAU101752 | 5522 | SAU1c0040_orf_85p | 12447 |
| S1M10000018D12 | 2105 | SAU100866 | 5344 | SAU1c0044_orf_100p | 12553 |
| S1M10000018E01 | 2106 | SAU101092 | 5381 | SAU1c0028_orf_9p | 12192 |
| S1M10000018E02 | 2107 | SAU100265 | 5249 | SAU1c0014_orf_11p | 12122 |
| S1M10000018E03 | 2108 | SAU102420 | 5665 | SAU1c0030_orf_20p | 12206 |
| S1M10000018E04 | 2109 | SAU102035 | 5592 | SAU1c0029_orf_50P | 12199 |
| S1M10000018E05 | 2110 | SAU100596 | 5295 | SAU1c0043_orf_63p | 12548 |
| S1M10000018E08 | 2111 | SAU100793 | 5329 | SAU1c0028_orf_52p | 12188 |
| S1M10000018E09 | 2112 | SAU301898 | 5904 | SAU3c1079_orf_1p | 13057 |
| S1M10000018E11 | 2113 | SAU101799 | 5539 | SAU1c0032_orf_19p | 12223 |
| S1M10000018E11 | 2113 | SAU101800 | 5540 | SAU1c0032_orf_20p | 12225 |
| S1M10000018E12 | 2114 | SAU200914 | 5796 | SAU2c0373_orf_2p | 12837 |
| S1M10000018F03 | 2115 | SAU100887 | 5350 | SAU1c0018_orf_15p | 12138 |
| S1M10000018F04 | 2116 | SAU102396 | 5660 | SAU1c0033_orf_43p | 12272 |
| S1M10000018F04 | 2116 | SAU301118 | 5886 | SAU3c1305_orf_3p | 13086 |
| S1M10000018F07 | 2117 | SAU102629 | 5720 | SAU1c0041_orf_71p | 12481 |
| S1M10000018F09 | 2118 | SAU101810 | 5549 | SAU1c0032_orf_28p | 12233 |
| S1M10000018F09 | 2118 | SAU300110 | 5865 | SAU3c0533_orf_2p | 13031 |
| S1M10000018F10 | 2119 | SAU100432 | 5271 | SAU1c0040_orf_88p | 12450 |
| S1M10000018F10 | 2119 | SAU100433 | 5272 | SAU1c0040_orf_87p | 12449 |
| S1M10000018F12 | 2120 | SAU201469 | 5816 | SAU2c0438_orf_6p | 12967 |
| S1M10000018G03 | 2121 | SAU101808 | 5548 | SAU1c0032_orf_27p | 12232 |
| S1M10000018G05 | 2122 | SAU101999 | 5585 | SAU1c0040_orf_101p | 12423 |
| S1M10000018G07 | 2123 | SAU101727 | 5516 | SAU1c0016_orf_6p | 12133 |
| S1M10000018G08 | 2124 | SAU102200 | 5611 | SAU1c0045_orf_168p | 12665 |
| S1M10000018G08 | 2124 | SAU102201 | 5612 | SAU1c0045_orf_169p | 12666 |
| S1M10000018G09 | 2125 | SAU102200 | 5611 | SAU1c0045_orf_168p | 12665 |
| S1M10000018G09 | 2125 | SAU102201 | 5612 | SAU1c0045_orf_169p | 12666 |
| S1M10000018G10 | 2126 | SAU100141 | 5236 | SAU1c0032_orf_8p | 12259 |
| S1M10000018G10 | 2126 | SAU102527 | 5693 | SAU1c0032_orf_9p | 12260 |
| S1M10000018G12 | 2127 | SAU200928 | 5798 | SAU2c0365_orf_5p | 12815 |
| S1M10000018H01 | 2128 | SAU101663 | 5506 | SAU1c0033_orf_14p | 12261 |
| S1M10000018H02 | 2129 | SAU101652 | 5503 | SAU1c0042_orf_123p | 12492 |
| S1M10000018H02 | 2129 | SAU101653 | 5504 | SAU1c0042_orf_124p | 12493 |
| S1M10000018H07 | 2130 | SAU102437 | 5670 | SAU1c0045_orf_33p | 12695 |
| S1M10000018H09 | 2131 | SAU101622 | 5496 | SAU1c0040_orf_27p | 12430 |
| S1M10000018H10 | 2132 | SAU100157 | 5237 | SAU1c0040_orf_81p | 12444 |
| S1M10000019A02 | 2133 | SAU103077 | 5759 | SAU1c0039_orf_44p | 12408 |
| S1M10000019A03 | 2134 | SAU102352 | 5650 | SAU1c0400_orf_38p | 12434 |
| S1M10000019A05 | 2135 | SAU201469 | 5816 | SAU2c0438_orf_6p | 12967 |
| S1M10000019A06 | 2136 | SAU101311 | 5419 | SAU1c0044_orf_126p | 12563 |
| S1M10000019A07 | 2137 | SAU101727 | 5516 | SAU1c0016_orf_6p | 12133 |
| S1M10000019A07 | 2137 | SAU101728 | 5517 | SAU1c0016_orf_5p | 12132 |
| S1M10000019A09 | 2138 | SAU102117 | 5603 | SAU1c0027_orf_6p | 12181 |
| S1M10000019A11 | 2139 | SAU102292 | 5638 | SAU1c0038_orf_10p | 12368 |
| S1M10000019A12 | 2140 | SAU102693 | 5731 | SAU1c0044_orf_58p | 12627 |
| S1M10000019A12 | 2140 | SAU102694 | 5732 | SAU1c0044_orf_59p | 12628 |
| S1M10000019B03 | 2141 | SAU101156 | 5386 | SAU1c0036_orf_12p | 12311 |
| S1M10000019B04 | 2142 | SAU100899 | 5351 | SAU1c0034_orf_11p | 12277 |
| S1M10000019B04 | 2142 | SAU100901 | 5352 | SAU1c0034_orf_13p | 12278 |
| S1M10000019B07 | 2143 | SAU100300 | 5253 | SAU1c0040_orf_90p | 12451 |
| S1M10000019B08 | 2144 | SAU102422 | 5666 | SAU1c0030_orf_22p | 12207 |
| S1M10000019B08 | 2144 | SAU102423 | 5667 | SAU1c0030_orf_23p | 12208 |
| S1M10000019B09 | 2145 | SAU100182 | 5241 | SAU1c0037_orf_82p | 12362 |
| S1M10000019B09 | 2145 | SAU100251 | 5248 | SAU1c0037_orf_83p | 12363 |
| S1M10000019B10 | 2146 | SAU101570 | 5482 | SAU1c0044_orf_209p | 12584 |
| S1M10000019B11 | 2147 | SAU100879 | 5345 | SAU1c0041_orf_82p | 12483 |
| S1M10000019B12 | 2148 | SAU101793 | 5534 | SAU1c0032_orf_14p | 12218 |
| S1M10000019C01 | 2149 | SAU100414 | 5270 | SAU1c0022_orf_24p | 12148 |
| S1M10000019C04 | 2150 | SAU103175 | 5764 | SAU1c0045_orf_269p | 12687 |
| S1M10000019C04 | 2150 | SAU301472 | 5897 | SAU3c1431_orf_4p | 13124 |
| S1M10000019C05 | 2151 | SAU101756 | 5524 | SAU1c0040_orf_82p | 12445 |
| S1M10000019C06 | 2152 | SAU101790 | 5531 | SAU1c0032_orf_11p | 12215 |
| S1M10000019C06 | 2152 | SAU101791 | 5532 | SAU1c0032_orf_12p | 12216 |
| S1M10000019C07 | 2153 | SAU101400 | 5444 | SAU1c0036_orf_35p | 12326 |
| S1M10000019C08 | 2154 | SAU202126 | 5844 | SAU2c0045_orf_1p | 12714 |
| S1M10000019C11 | 2155 | SAU100301 | 5254 | SAU1c0040_orf_91p | 12452 |
| S1M10000019C12 | 2156 | SAU102117 | 5603 | SAU1c0027_orf_6p | 12181 |
| S1M10000019D01 | 2157 | SAU102270 | 5631 | SAU1c0032_orf_65p | 12253 |
| S1M10000019D02 | 2158 | SAU101145 | 5384 | SAU1c0035_orf_43p | 12299 |
| S1M10000019D04 | 2159 | SAU102292 | 5638 | SAU1c0038_orf_10p | 12368 |
| S1M10000019D05 | 2160 | SAU101400 | 5444 | SAU1c0036_orf_35p | 12326 |
| S1M10000019D06 | 2161 | SAU102526 | 5692 | SAU1c0045_orf_299p | 12691 |
| S1M10000019D07 | 2162 | SAU301898 | 5904 | SAU3c1079_orf_1p | 13057 |
| S1M10000019D09 | 2163 | SAU102639 | 5724 | #N/A | #N/A |
| S1M10000019D12 | 2164 | SAU101805 | 5545 | SAU1c0032_orf_24p | 12229 |
| S1M10000019E01 | 2165 | SAU100961 | 5360 | SAU1c0044_orf_83p | 12638 |
| S1M10000019E01 | 2165 | SAU100962 | 5361 | SAU1c0044_orf_84p | 12639 |
| S1M10000019E02 | 2166 | SAU101624 | 5497 | SAU1c0040_orf_25p | 12429 |
| S1M10000019E07 | 2167 | SAU102352 | 5650 | SAU1c0040_orf_38p | 12434 |
| S1M10000019F01 | 2168 | SAU102241 | 5617 | SAU1c0043_orf_25p | 12539 |
| S1M10000019F05 | 2169 | SAU101612 | 5493 | SAU1c0044_orf_7p | 12637 |
| S1M10000019F05 | 2169 | SAU202945 | 5857 | SAU2c0394_orf_7p | 12868 |
| S1M10000019F06 | 2170 | SAU101864 | 5562 | SAU1c0044_orf_163p | 12572 |
| S1M10000019F08 | 2171 | SAU101571 | 5483 | SAU1c0044_orf_210p | 12585 |
| S1M10000019F09 | 2172 | SAU100414 | 5270 | SAU1c0022_orf_24p | 12148 |
| S1M10000019F11 | 2173 | SAU101242 | 5404 | SAU1c0044_orf_18p | 12578 |
| S1M10000019G04 | 2174 | SAU101793 | 5534 | SAU1c0032_orf_14p | 12218 |
| S1M10000019G07 | 2175 | SAU100522 | 5284 | SAU1c0044_orf_249p | 12599 |
| S1M10000019G09 | 2176 | SAU100300 | 5253 | SAU1c0040_orf_90p | 12451 |
| S1M10000019G10 | 2177 | SAU101235 | 5400 | SAU1c0044_orf_11p | 12561 |
| S1M10000019G10 | 2177 | SAU101236 | 5401 | SAU1c0044_orf_12p | 12564 |
| S1M10000019G11 | 2178 | SAU101802 | 5542 | SAU1c0032_orf_22p | 12227 |
| S1M10000019H05 | 2179 | SAU101802 | 5542 | SAU1c0032_orf_22p | 12227 |
| S1M10000019H05 | 2179 | SAU101803 | 5543 | SAU1c0032_orf_23p | 1228 |
| S1M10000019H08 | 2180 | SAU102449 | 5674 | SAU1c0045_orf_22p | 12677 |
| S1M10000020A05 | 2181 | SAU101868 | 5565 | SAU1c0036_orf_23p | 12320 |
| S1M10000020A06 | 2182 | SAU101801 | 5541 | #N/A | #N/A |
| S1M10000020A07 | 2183 | SAU101567 | 5481 | SAU1c0022_orf_10p | 12144 |
| S1M10000020A07 | 2183 | SAU200030 | 5772 | SAU2c0282_orf_3p | 12745 |
| S1M10000020A11 | 2184 | SAU102437 | 5670 | SAU1c0045_orf_33p | 12695 |
| S1M10000020A12 | 2185 | SAU101907 | 5574 | SAU1c0040_orf_79p | 12442 |
| 51M10000020B02 | 2186 | SAU100475 | 5276 | SAU1c0036_orf_61p | 12337 |
| S1M10000020B03 | 2187 | SAU100059 | 5224 | SAU1c0045_orf_10p | 12652 |
| S1M10000020B05 | 2188 | SAU301133 | 5887 | SAU3c1311_orf_3p | 13087 |
| S1M10000020B06 | 2189 | SAU100747 | 5320 | SAU1c0044_orf_235p | 12597 |
| S1M10000020B07 | 2190 | SAU102433 | 5668 | SAU1c0045_orf_37p | 12701 |
| S1M10000020B09 | 2191 | SAU101371 | 5435 | SAU1c0033_orf_7p | 12275 |
| S1M10000020B12 | 2192 | SAU102143 | 5607 | SAU1c0041_orf_14p | 12458 |
| S1M10000020C09 | 2193 | SAU101545 | 5474 | SAU1c0037_orf_132p | 12348 |
| S1M10000020C10 | 2194 | SAU101799 | 5539 | SAU1c0032_orf_19p | 12223 |
| S1M10000020C10 | 2194 | SAU101800 | 5540 | SAU1c0032_orf_20p | 12225 |
| S1M10000020C11 | 2195 | SAU101452 | 5455 | SAU1c0045_orf_247p | 12684 |
| S1M10000020D03 | 2196 | SAU101752 | 5522 | SAU1c0040_orf_85p | 12447 |
| S1M10000020D04 | 2197 | SAU102481 | 5685 | SAU1c0039_orf_99p | 12422 |
| S1M10000020D06 | 2198 | SAU102578 | 5701 | SAU1c0039_orf_61p | 12411 |
| S1M10000020D07 | 2199 | SAU100198 | 5243 | SAU1c0009_orf_1p | 12120 |
| S1M10000020D08 | 2200 | SAU100547 | 5290 | SAU1c0032_orf_3p | 12240 |
| S1M10000020D09 | 2201 | SAU102939 | 5747 | #N/A | #N/A |
| S1M10000020D12 | 2202 | SAU200006 | 5770 | SAU2c0157_orf_1p | 12723 |
| S1M10000020E01 | 2203 | SAU200006 | 5770 | SAU2c0157_orf_1p | 12723 |
| S1M10000020E03 | 2204 | SAU100140 | 5235 | SAU1c0032_orf_7p | 12258 |
| S1M10000020E04 | 2205 | SAU101805 | 5545 | SAU1c0032_orf_24p | 12229 |
| S1M10000020E06 | 2206 | SAU102162 | 5609 | SAU1c0041_orf_27p | 12462 |
| S1M10000020E08 | 2207 | SAU101756 | 5524 | SAU1c0040_orf_82p | 12445 |
| S1M10000020E11 | 2208 | SAU101876 | 5567 | SAU1c0025_orf_9p | 12169 |
| S1M10000020E12 | 2209 | SAU200657 | 5789 | #N/A | #N/A |
| S1M10000020F01 | 2210 | SAU101592 | 5490 | SAU1c0039_orf_37p | 12406 |
| S1M10000020F05 | 2211 | SAU100547 | 5290 | SAU1c0032_orf_3p | 12240 |
| S1M10000020F06 | 2212 | SAU101652 | 5503 | SAU1c0042_orf_123p | 12492 |
| S1M10000020F06 | 2212 | SAU101653 | 5504 | SAU1c0042_orf_124p | 12493 |
| S1M10000020F07 | 2213 | SAU200731 | 5793 | SAU2c0352_orf_2p | 12808 |
| S1M10000020F09 | 2214 | SAU100114 | 5228 | SAU1c0043_orf_225p | 12535 |
| S1M10000020F11 | 2215 | SAU101663 | 5506 | SAU1c0033_orf_14p | 12261 |
| S1M10000020F11 | 2215 | SAU101664 | 5507 | SAU1c0033_orf_15p | 12262 |
| S1M10000020F12 | 2216 | SAU100745 | 5319 | SAU1c0044_orf_233p | 12596 |
| S1M10000020G01 | 2217 | SAU102905 | 5742 | SAU1c0033_orf_45p | 12273 |
| S1M10000020G05 | 2218 | SAU100114 | 5228 | SAU1c0043_orf_225p | 12535 |
| S1M10000020G07 | 2219 | SAU100114 | 5228 | SAU1c0043_orf_225p | 12535 |
| S1M10000020G08 | 2220 | SAU101652 | 5503 | SAU1c0042_orf_123p | 12492 |
| S1M10000020G09 | 2221 | SAU101652 | 5503 | SAU1c0042_orf_123p | 12492 |
| S1M10000020G10 | 2222 | SAU101807 | 5547 | SAU1c0032_orf_26p | 12231 |
| S1M10000020G10 | 2222 | SAU101808 | 5548 | SAU1c0032_orf_27p | 12232 |
| S1M10000020G11 | 2223 | SAU101592 | 5490 | SAU1c0039_orf_37p | 12406 |
| S1M10000020G12 | 2224 | SAU100865 | 5343 | SAU1c0044_orf_99p | 12648 |
| S1M10000020H01 | 2225 | SAU202039 | 5843 | SAU2c0452_orf_20p | 13009 |
| S1M10000020H02 | 2226 | SAU101754 | 5523 | SAU1c0040_orf_84p | 12446 |
| S1M10000020H04 | 2227 | SAU101791 | 5532 | SAU1c0032_orf_12p | 12216 |
| S1M10000020H06 | 2228 | SAU101541 | 5472 | SAU1c0037_orf_128p | 12344 |
| S1M10000020H08 | 2229 | SAU201558 | 5823 | SAU2c0434_orf_5p | 12954 |
| S1M10000020H10 | 2230 | SAU101754 | 5523 | SAU1c0040_orf_84p | 12446 |
| S1M10000020H11 | 2231 | SAU100053 | 5222 | SAU1c0020_orf_1p | 12143 |
| S1M10000021A04 | 2232 | SAU200752 | 5795 | SAU2c0354_orf_5p | 12809 |
| S1M10000021A04 | 2232 | SAU300975 | 5880 | SAU3c1240_orf_3p | 13075 |
| S1M10000021A05 | 2233 | SAU101408 | 5445 | SAU1c0035_orf_93p | 12308 |
| S1M10000021A06 | 2234 | SAU200928 | 5798 | SAU2c0365_orf_5p | 12815 |
| S1M10000021A07 | 2235 | SAU100496 | 5279 | SAU1c0041_orf_83p | 12484 |
| S1M10000021A07 | 2235 | SAU301004 | 5882 | SAU3c1255_orf_1p | 13079 |
| S1M10000021A08 | 2236 | SAU101183 | 5390 | SAU1c0035_orf_79p | 12304 |
| S1M10000021A09 | 2237 | SAU102933 | 5744 | SAU1c0039_orf_62p | 12412 |
| S1M10000021A09 | 2237 | SAU201184 | 5805 | SAU2c0351_orf_19p | 12807 |
| S1M10000021A10 | 2238 | SAU101545 | 5474 | SAU1c0037_orf_132p | 12348 |
| S1M10000021B05 | 2239 | SAU100139 | 5234 | SAU1c0032_orf_6p | 12255 |
| S1M10000021B05 | 2239 | SAU102602 | 5708 | SAU1c0032_orf_5p | 12249 |
| S1M10000021B06 | 2240 | SAU101752 | 5522 | SAU1c0040_orf_85p | 12447 |
| S1M1000001307 | 2241 | SAU101632 | 5499 | SAU1c0039_orf_3p | 12407 |
| S1M10000021B10 | 2242 | SAU101772 | 5526 | SAU1c0037_orf_34p | 12351 |
| S1M10000021C04 | 2243 | SAU200928 | 5798 | SAU2c0365_orf_5p | 12815 |
| S1M10000021C05 | 2244 | SAU101271 | 5411 | SAU1c0037_orf_90p | 12366 |
| S1M10000021C07 | 2245 | SAU202968 | 5858 | SAU2c0407_orf_2p | 12886 |
| S1M10000021C08 | 2246 | SAU102575 | 5700 | SAU1c0044_orf_283p | 12609 |
| S1M10000021C10 | 2247 | SAU101320 | 5420 | SAU1c0015_orf_16p | 12128 |
| S1M10000021C11 | 2248 | SAU200006 | 5770 | SAU2c0157_orf_1p | 12723 |
| S1M10000021C12 | 2249 | SAU101726 | 5515 | SAU1c0016_orf_7p | 12134 |
| S1M10000021D01 | 2250 | SAU102503 | 5691 | SAU1c0045_orf_274p | 12690 |
| S1M10000021D03 | 2251 | SAU101271 | 5411 | SAU1c0037_orf_90p | 12366 |
| S1M10000021D03 | 2251 | SAU101286 | 5413 | SAU1c0034_orf_67p | 12292 |
| S1M10000021D04 | 2252 | SAU100858 | 5341 | SAU1c0038_orf_86p | 12401 |
| S1M10000021D04 | 2252 | SAU100859 | 5342 | SAU1c0038_orf_87p | 12402 |
| S1M10000021D06 | 2253 | SAU100865 | 5343 | SAU1c0044_orf_99p | 12648 |
| S1M10000021D09 | 2254 | SAU101868 | 5565 | SAU1c0036_orf_23p | 12320 |
| S1M10000021D10 | 2255 | SAU100714 | 5312 | SAU1c0044_orf_74p | 12635 |
| S1M10000021E01 | 2256 | SAU101655 | 5505 | SAU1c0042_orf_125p | 12494 |
| S1M10000021E02 | 2257 | SAU102200 | 5611 | SAU1c0045_orf_168p | 12665 |
| S1M10000021E02 | 2257 | SAU102201 | 5612 | SAU1c0045_orf_169p | 12666 |
| S1M10000021E03 | 2258 | SAU101857 | 5560 | SAU1c0044_orf_156p | 12569 |
| S1M10000021E05 | 2259 | SAU101777 | 5527 | SAU1c0037_orf_39p | 12352 |
| S1M10000021E06 | 2260 | SAU102663 | 5727 | SAU1c0024_orf_2p | 12158 |
| S1M10000021E09 | 2261 | SAU200006 | 5770 | SAU2c0157_orf_1p | 12723 |
| S1M10000021E12 | 2262 | SAU102292 | 5638 | SAU1c0038_orf_10p | 12368 |
| S1M10000021F02 | 2263 | SAU102059 | 5597 | SAU1c0034_orf_51p | 12286 |
| S1M10000021F04 | 2264 | SAU100139 | 5234 | SAU1c0032_orf_6p | 12255 |
| S1M10000021F04 | 2264 | SAU102602 | 5708 | SAU1c0032_orf_5p | 12249 |
| S1M10000021F05 | 2265 | SAU102059 | 5597 | SAU1c0034_orf_51p | 12286 |
| S1M10000021F06 | 2266 | SAU101235 | 5400 | SAU1c0044_orf_11p | 12561 |
| S1M10000021F07 | 2267 | SAU101383 | 5438 | SAU1c0022_orf_20p | 12147 |
| S1M10000021F09 | 2268 | SAU102059 | 5597 | SAU1c0034_orf_51p | 12286 |
| S1M10000021F09 | 2268 | SAU301465 | 5896 | SAU3c1429_orf_4p | 13121 |
| S1M10000021F11 | 2269 | SAU101371 | 5435 | SAU1c0033_orf_7p | 12275 |
| S1M10000021G01 | 2270 | SAU200468 | 5781 | SAU2c0429_orf_19p | 12937 |
| S1M10000021G03 | 2271 | SAU301357 | 5893 | SAU3c1394_orf_2p | 13111 |
| S1M10000021G08 | 2272 | SAU100714 | 5312 | SAU1c0044_orf_74p | 12635 |
| S1M10000021H04 | 2273 | SAU100139 | 5234 | SAU1c0032_orf_6p | 12255 |
| S1M10000021H04 | 2273 | SAU102602 | 5708 | SAU1c0032_orf_5p | 12249 |
| S1M10000021H05 | 2274 | SAU300131 | 5866 | SAU3c0560_orf_2p | 13034 |
| S1M10000021H07 | 2275 | SAU101806 | 5546 | SAU1c0032_orf_25p | 12230 |
| S1M10000021H08 | 2276 | SAU102059 | 5597 | SAU1c0034_orf_51p | 12286 |
| S1M10000021H11 | 2277 | SAU101543 | 5473 | SAU1c0037_orf_130p | 12346 |
| S1M10000022A02 | 2278 | SAU100865 | 5343 | SAU1c0044_orf_99p | 12648 |
| S1M10000022A02 | 2278 | SAU301230 | 5890 | SAU3c1347_orf_6p | 13092 |
| S1M10000022A03 | 2279 | SAU201197 | 5806 | SAU2c0429_orf_2p | 12938 |
| S1M10000022A05 | 2280 | SAU101807 | 5547 | SAU1c0032_orf_26p | 12231 |
| S1M10000022A08 | 2281 | SAU101365 | 5432 | SAU1c0044_orf_112p | 12556 |
| S1M10000022A09 | 2282 | SAU102939 | 5747 | #N/A | #N/A |
| S1M10000022A12 | 2283 | SAU101868 | 5565 | SAU1c0036_orf_23p | 12320 |
| S1M10000022B02 | 2284 | SAU100865 | 5343 | SAU1c0044_orf_99p | 12648 |
| S1M10000022B02 | 2284 | SAU301230 | 5890 | SAU3c1347_orf_6p | 13092 |
| S1M10000022B03 | 2285 | SAU200468 | 5781 | SAU2c0429_orf_19p | 12937 |
| S1M10000022B05 | 2286 | SAU100920 | 5354 | SAU1c0038_orf_75p | 12395 |
| S1M10000022B06 | 2287 | SAU100714 | 5312 | SAU1c0044_orf_74p | 12635 |
| S1M10000022B08 | 2288 | SAU102292 | 5638 | SAU1c0038_orf_10p | 12368 |
| S1M10000022B09 | 2289 | SAU102939 | 5747 | #N/A | #N/A |
| S1M10000022B10 | 2290 | SAU101546 | 5475 | SAU1c0037_orf_133p | 12349 |
| S1M10000022B11 | 2291 | SAU101726 | 5515 | SAU1c0016_orf_7p | 12134 |
| S1M10000022B12 | 2292 | SAU101868 | 5565 | SAU1c0036_orf_23p | 12320 |
| S1M10000022C02 | 2293 | SAU102059 | 5597 | SAU1c0034_orf_51p | 12286 |
| S1M10000022C03 | 2294 | SAU101791 | 5532 | SAU1c0032_orf_12p | 12216 |
| S1M10000022C04 | 2295 | SAU100714 | 5312 | SAU1c0044_orf_74p | 12635 |
| S1M10000022C06 | 2296 | SAU100246 | 5247 | SAU1c0042_orf_130p | 12496 |
| S1M10000022C06 | 2296 | SAU300998 | 5881 | SAU3c1253_orf_3p | 13077 |
| S1M10000022C07 | 2297 | SAU101546 | 5475 | SAU1c0037_orf_133p | 12349 |
| S1M10000022C08 | 2298 | SAU100528 | 5286 | SAU1c0042_orf_87p | 12507 |
| S1M10000022C08 | 2298 | SAU103115 | 5760 | SAU1c0042_orf_88p | 12508 |
| S1M10000022C11 | 2299 | SAU102059 | 5597 | SAU1c0034_orf_51p | 12286 |
| S1M10000022D03 | 2300 | SAU101805 | 5545 | SAU1c0032_orf_24p | 12229 |
| S1M10000022D05 | 2301 | SAU101777 | 5527 | SAU1c0037_orf_39p | 12352 |
| S1M10000022D06 | 2302 | SAU100921 | 5355 | SAU1c0038_orf_76p | 12396 |
| S1M10000022D07 | 2303 | SAU101543 | 5473 | SAU1c0037_orf_130p | 12346 |
| S1M10000022D08 | 2304 | SAU101189 | 5392 | SAU1c0033_orf_25p | 12264 |
| S1M10000022D09 | 2305 | SAU101726 | 5515 | SAU1c0016_orf_7p | 12134 |
| S1M10000022D11 | 2306 | SAU101447 | 5454 | SAU1c0045_orf_244p | 12683 |
| S1M10000022E01 | 2307 | SAU200601 | 5787 | #N/A | #N/A |
| S1M10000022E03 | 2308 | SAU200468 | 5781 | SAU2c0429_orf_19p | 12937 |
| S1M10000022E05 | 2309 | SAU301465 | 5896 | SAU3c1429_orf_4p | 13121 |
| S1M10000022E09 | 2310 | SAU101235 | 5400 | SAU1c0044_orf_11p | 12561 |
| S1M10000022E09 | 2310 | SAU101236 | 5401 | SAU1c0044_orf_12p | 12564 |
| S1M10000022F04 | 2311 | SAU101592 | 5490 | SAU1c0039_orf_37p | 12406 |
| S1M10000022F06 | 2312 | SAU101868 | 5565 | SAU1c0036_orf_23p | 12320 |
| S1M10000022F07 | 2313 | SAU102117 | 5603 | SAU1c0027_orf_6p | 12181 |
| S1M10000022F08 | 2314 | SAU100414 | 5270 | SAU1c0022_orf_24p | 12148 |
| S1M10000022F11 | 2315 | SAU101592 | 5490 | SAU1c0039_orf_37p | 12406 |
| S1M10000022G03 | 2316 | SAU301465 | 5896 | SAU3c1429_orf_4p | 13121 |
| S1M10000022G04 | 2317 | SAU101777 | 5527 | SAU1c0037_orf_39p | 12352 |
| S1M10000022G07 | 2318 | SAU100414 | 5270 | SAU1c0022_orf_24p | 12148 |
| S1M10000022G08 | 2319 | SAU100557 | 5291 | SAU1c0044_orf_132p | 12565 |
| S1M10000022G12 | 2320 | SAU101546 | 5475 | SAU1c0037_orf_133p | 12349 |
| S1M10000022H03 | 2321 | SAU101006 | 5367 | SAU1c0028_orf_59p | 12190 |
| S1M10000022H05 | 2322 | SAU101814 | 5551 | SAU1c0032_orf_32p | 12237 |
| S1M10000022H06 | 2323 | SAU200928 | 5798 | SAU2c0365_orf_5p | 12815 |
| S1M10000022H07 | 2324 | SAU100866 | 5344 | SAU1c0044_orf_100p | 12553 |
| S1M10000022H08 | 2325 | SAU100887 | 5350 | SAU1c0018_orf_15p | 12138 |
| S1M10000022H11 | 2326 | SAU101610 | 5492 | SAU1c0044_orf_5p | 12629 |
| S1M10000023A05 | 2327 | SAU301465 | 5896 | SAU3c1429_orf_4p | 13121 |
| S1M10000023A09 | 2328 | SAU101340 | 5423 | SAU1c0038_orf_82p | 12400 |
| S1M10000023A11 | 2329 | SAU100547 | 5290 | SAU1c0032_orf_3p | 12240 |
| S1M10000023A12 | 2330 | SAU101651 | 5502 | SAU1c0042_orf_122p | 12491 |
| S1M10000023A12 | 2330 | SAU101652 | 5503 | SAU1c0042_orf_123p | 12492 |
| S1M10000023B01 | 2331 | SAU100886 | 5349 | SAU1c0018_orf_16p | 12139 |
| S1M10000023B03 | 2332 | SAU101652 | 5503 | SAU1c0042_orf_123p | 12492 |
| S1M10000023B03 | 2332 | SAU101653 | 5504 | SAU1c0042_orf_124p | 12493 |
| S1M10000023B07 | 2333 | SAU101857 | 5560 | SAU1c0044_orf_156p | 12569 |
| S1M10000023B08 | 2334 | SAU100140 | 5235 | SAU1c0032_orf_7p | 12258 |
| S1M10000023B08 | 2334 | SAU100141 | 5236 | SAU1c0032_orf_8p | 12259 |
| S1M10000023B09 | 2335 | SAU101340 | 5423 | SAU1c0038_orf_82p | 12400 |
| S1M10000023B10 | 2336 | SAU102578 | 5701 | SAU1c0039_orf_61p | 12411 |
| S1M10000023B11 | 2337 | SAU102613 | 5715 | SAU1c0041_orf_55p | 12475 |
| S1M10000023B12 | 2338 | SAU202174 | 5845 | SAU2c0412_orf_3p | 12895 |
| S1M10000023B12 | 2338 | SAU301148 | 5888 | #N/A | #N/A |
| S1M10000023C02 | 2339 | SAU100140 | 5235 | SAU1c0032_orf_7p | 12258 |
| S1M10000023C02 | 2339 | SAU100141 | 5236 | SAU1c0032_orf_8p | 12259 |
| S1M10000023C10 | 2340 | SAU102554 | 5699 | SAU1c0045_orf_209p | 12673 |
| S1M10000023C11 | 2341 | SAU102352 | 5650 | SAU1c0040_orf_38p | 12434 |
| S1M10000023C12 | 2342 | SAU100077 | 5226 | SAU1c0043_orf_178p | 12520 |
| S1M10000023D01 | 2343 | SAU100964 | 5363 | SAU1c0044_orf_86p | 12641 |
| S1M10000023D03 | 2344 | SAU101996 | 5584 | SAU1c0040_orf_99p | 12456 |
| S1M10000023D04 | 2345 | SAU102602 | 5708 | SAU1c0032_orf_5p | 12249 |
| S1M10000023D07 | 2346 | SAU101543 | 5473 | SAU1c0037_orf_130p | 12346 |
| S1M10000023D08 | 2347 | SAU100887 | 5350 | SAU1c0018_orf_15p | 12138 |
| S1M10000023D09 | 2348 | SAU100547 | 5290 | SAU1c0032_orf_3p | 12240 |
| S1M10000023D10 | 2349 | SAU100963 | 5362 | SAU1c0044_orf_85p | 12640 |
| S1M10000023D12 | 2350 | SAU102292 | 5638 | SAU1c0038_orf_10p | 12368 |
| S1M10000023E01 | 2351 | SAU101752 | 5522 | SAU1c0040_orf_85p | 12447 |
| S1M10000023E04 | 2352 | SAU102059 | 5597 | SAU1c0034_orf_51p | 12286 |
| S1M10000023E07 | 2353 | SAU101543 | 5473 | SAU1c0037_orf_130p | 12346 |
| S1M10000023E10 | 2354 | SAU203293 | 5862 | SAU2c0441_orf_21p | 12979 |
| S1M10000023E11 | 2355 | SAU102292 | 5638 | SAU1c0038_orf_10p | 12368 |
| S1M10000023F04 | 2356 | SAU101736 | 5518 | SAU1c0043_orf_166p | 12519 |
| S1M10000023F04 | 2356 | SAU101737 | 5519 | SAU1c0043_orf_165p | 12518 |
| S1M10000023F07 | 2357 | SAU100546 | 5289 | SAU1c0032_orf_2p | 12235 |
| S1M10000023F08 | 2358 | SAU102883 | 5741 | SAU1c0045_orf_38p | 12702 |
| S1M10000023F10 | 2359 | SAU102352 | 5650 | SAU1c0040_orf_38p | 12434 |
| S1M10000023F11 | 2360 | SAU100617 | 5300 | SAU1c0035_orf_102p | 12295 |
| S1M10000023F12 | 2361 | SAU102352 | 5650 | SAU1c0040_orf_38p | 12434 |
| S1M10000023G02 | 2362 | SAU301465 | 5896 | SAU3c1429_orf_4p | 13121 |
| S1M10000023G03 | 2363 | SAU101996 | 5584 | SAU1c0040_orf_99p | 12456 |
| S1M10000023G06 | 2364 | SAU100887 | 5350 | SAU1c0018_orf_15p | 12138 |
| S1M10000023G07 | 2365 | SAU301054 | 5884 | #N/A | #N/A |
| S1M10000023G08 | 2366 | SAU100964 | 5363 | SAU1c0044_orf_86p | 12641 |
| S1M10000023G09 | 2367 | SAU101968 | 5581 | SAU1c0028_orf_43p | 12187 |
| S1M10000023G11 | 2368 | SAU102613 | 5715 | SAU1c0041_orf_55p | 12475 |
| S1M10000023H02 | 2369 | SAU101996 | 5584 | SAU1c0040_orf_99p | 12456 |
| S1M10000023H06 | 2370 | SAU100158 | 5238 | SAU1c0040_orf_80p | 12443 |
| S1M10000023H07 | 2371 | SAU100300 | 5253 | SAU1c0040_orf_90p | 12451 |
| S1M10000023H09 | 2372 | SAU101340 | 5423 | SAU1c0038_orf_82p | 12400 |
| S1M10000023H10 | 2373 | SAU101365 | 5432 | SAU1c0044_orf_112p | 12556 |
| S1M10000024A02 | 2374 | SAU101798 | 5538 | SAU1c0032_orf_18p | 12222 |
| S1M10000024A04 | 2375 | SAU201571 | 5824 | SAU2c0447_orf_17p | 12997 |
| S1M10000024A07 | 2376 | SAU100414 | 5270 | SAU1c0022_orf_24p | 12148 |
| S1M10000024A08 | 2377 | SAU101231 | 5399 | SAU1c0035_orf_6p | 12303 |
| S1M10000024A11 | 2378 | SAU103226 | 5768 | SAU1c0045_orf_95p | 12713 |
| S1M10000024B05 | 2379 | SAU102418 | 5664 | SAU1c0030_orf_18p | 12205 |
| S1M10000024B06 | 2380 | SAU100158 | 5238 | SAU1c0040_orf_80p | 12443 |
| S1M10000024B08 | 2381 | SAU100601 | 5296 | SAU1c0044_orf_313p | 12616 |
| S1M10000024B09 | 2382 | SAU200468 | 5781 | SAU2c0429_orf_19p | 12937 |
| S1M10000024B10 | 2383 | SAU101265 | 5407 | #N/A | #N/A |
| S1M10000024C02 | 2384 | SAU101197 | 5393 | SAU1c0035_orf_60p | 12300 |
| S1M10000024C04 | 2385 | SAU101862 | 5561 | SAU1c0044_orf_161p | 12571 |
| S1M10000024C07 | 2386 | SAU101039 | 5373 | SAU1c0043_orf_181p | 12522 |
| S1M10000024D02 | 2387 | SAU100414 | 5270 | SAU1c0022_orf_24p | 12148 |
| S1M10000024D03 | 2388 | SAU100714 | 5312 | SAU1c0044_orf_74p | 12635 |
| S1M10000024D10 | 2389 | SAU100140 | 5235 | SAU1c0032_orf_7p | 12258 |
| S1M10000024D10 | 2389 | SAU100141 | 5236 | SAU1c0032_orf_8p | 12259 |
| S1M10000024D11 | 2390 | SAU101198 | 5394 | SAU1c0035_orf_61p | 12301 |
| S1M10000024E03 | 2391 | SAU201571 | 5824 | SAU2c0447_orf_17p | 12997 |
| S1M10000024E05 | 2392 | SAU101800 | 5540 | SAU1c0032_orf_20p | 12225 |
| S1M10000024E05 | 2392 | SAU101801 | 5541 | #N/A | #N/A |
| S1M10000024E06 | 2393 | SAU102418 | 5664 | SAU1c0030_orf_18p | 12205 |
| S1M10000024E07 | 2394 | SAU101039 | 5373 | SAU1c0043_orf_181p | 12522 |
| S1M10000024E08 | 2395 | SAU100414 | 5270 | SAU1c0022_orf_24p | 12148 |
| S1M10000024F02 | 2396 | SAU101447 | 5454 | SAU1c0045_orf_244p | 12683 |
| S1M10000024F03 | 2397 | SAU102992 | 5752 | SAU1c0044_orf_60p | 12630 |
| S1M10000024F05 | 2398 | SAU201197 | 5806 | SAU2c0429_orf_2p | 12938 |
| S1M10000024F08 | 2399 | SAU101726 | 5515 | SAU1c0016_orf_7p | 12134 |
| S1M10000024F10 | 2400 | SAU200468 | 5781 | SAU2c0429_orf_19p | 12937 |
| S1M10000024G05 | 2401 | SAU101800 | 5540 | SAU1c0032_orf_20p | 12225 |
| S1M10000024G05 | 2401 | SAU101801 | 5541 | #N/A | #N/A |
| S1M10000024G06 | 2402 | SAU102418 | 5664 | SAU1c0030_orf_18p | 12205 |
| S1M10000024G07 | 2403 | SAU102334 | 5645 | SAU1c0045_orf_144p | 12658 |
| S1M10000024G08 | 2404 | SAU101632 | 5499 | SAU1c0039_orf_3p | 12407 |
| S1M10000024G10 | 2405 | SAU202176 | 5846 | SAU2c0412_orf_3p | 12895 |
| S1M10000024G12 | 2406 | SAU100141 | 5236 | SAU1c0032_orf_8p | 12259 |
| S1M10000024H02 | 2407 | SAU201571 | 5824 | SAU2c0447_orf_17p | 12997 |
| S1M10000024H04 | 2408 | SAU100770 | 5324 | #N/A | #N/A |
| S1M10000024H07 | 2409 | SAU200725 | 5792 | SAU2c0428_orf_20p | 12933 |
| S1M10000024H08 | 2410 | SAU102002 | 5587 | SAU1c0040_orf_103p | 12425 |
| S1M10000024H08 | 2410 | SAU102003 | 5588 | SAU1c0040_orf_104p | 12426 |
| S1M10000025A03 | 2411 | SAU101247 | 5405 | SAU1c0043_orf_136p | 12512 |
| S1M10000025A08 | 2412 | SAU102766 | 5735 | #N/A | #N/A |
| S1M10000025A08 | 2412 | SAU201236 | 5808 | SAU2c0409_orf_10p | 12891 |
| S1M10000025A08 | 2412 | SAU300338 | 5871 | #N/A | #N/A |
| S1M10000025A09 | 2413 | SAU102292 | 5638 | SAU1c0038_orf_10p | 12368 |
| S1M10000025A10 | 2414 | SAU101455 | 5456 | SAU1c0045_orf_250p | 12686 |
| S1M10000025A10 | 2414 | SAU200916 | 5797 | SAU2c0373_orf_4p | 12838 |
| S1M10000025A10 | 2414 | SAU301620 | 5899 | SAU3c1478_orf_2p | 13140 |
| S1M10000025B01 | 2415 | SAU101655 | 5505 | SAU1c0042_orf_125p | 12494 |
| S1M10000025B02 | 2416 | SAU101808 | 5548 | SAU1c0032_orf_27p | 12232 |
| S1M10000025B03 | 2417 | SAU101385 | 5439 | SAU1c0038_orf_50p | 12385 |
| S1M10000025B05 | 2418 | SAU101455 | 5456 | SAU1c0045_orf_250p | 12686 |
| S1M10000025B05 | 2418 | SAU200916 | 5797 | SAU2c0373_orf_4p | 12838 |
| S1M10000025B05 | 2418 | SAU301620 | 5899 | SAU3c1478_orf_2p | 13140 |
| S1M10000025B06 | 2419 | SAU101545 | 5474 | SAU1c0037_orf_132p | 12348 |
| S1M10000025B09 | 2420 | SAU200928 | 5798 | SAU2c0365_orf_5p | 12815 |
| S1M10000025B12 | 2421 | SAU101791 | 5532 | SAU1c0032_orf_12p | 12216 |
| S1M10000025C01 | 2422 | SAU102292 | 5638 | SAU1c0038_orf_10p | 12368 |
| S1M10000025C03 | 2423 | SAU100139 | 5234 | SAU1c0032_orf_6p | 12255 |
| S1M10000025C05 | 2424 | SAU100139 | 5234 | SAU1c0032_orf_6p | 12255 |
| S1M10000025C09 | 2425 | SAU100793 | 5329 | SAU1c0028_orf_52p | 12188 |
| S1M10000025C09 | 2425 | SAU301433 | 5895 | SAU3c1420_orf_2p | 13118 |
| S1M10000025C10 | 2426 | SAU200928 | 5798 | SAU2c0365_orf_5p | 12815 |
| S1M10000025C11 | 2427 | SAU102117 | 5603 | SAU1c0027_orf_6p | 12181 |
| S1M10000025D01 | 2428 | SAU102117 | 5603 | SAU1c0027_orf_6p | 12181 |
| S1M10000025D03 | 2429 | SAU101771 | 5525 | SAU1c0037_orf_33p | 12350 |
| S1M10000025D03 | 2429 | SAU101772 | 5526 | SAU1c0037_orf_34p | 12351 |
| S1M10000025D04 | 2430 | SAU100970 | 5365 | SAU1c0043_orf_197p | 12529 |
| S1M10000025D06 | 2431 | SAU101543 | 5473 | SAU1c0037_orf_130p | 12346 |
| S1M10000025D08 | 2432 | SAU102598 | 5705 | SAU1c0041_orf_43p | 12464 |
| S1M10000025D08 | 2432 | SAU102599 | 5706 | SAU1c0041_orf_45p | 12466 |
| S1M10000025D08 | 2432 | SAU103191 | 5765 | SAU1c0041_orf_44p | 12465 |
| S1M10000025D09 | 2433 | SAU100522 | 5284 | SAU1c0044_orf_249p | 12599 |
| S1M10000025D10 | 2434 | SAU102200 | 5611 | SAU1c0045_orf_168p | 12665 |
| S1M10000025D10 | 2434 | SAU102201 | 5612 | SAU1c0045_orf_169p | 12666 |
| S1M10000025E01 | 2435 | SAU102117 | 5603 | SAU1c0027_orf_6p | 12181 |
| S1M10000025E04 | 2436 | SAU100389 | 5266 | SAU1c0034_orf_14p | 12279 |
| S1M10000025E09 | 2437 | SAU102117 | 5603 | SAU1c0027_orf_6p | 12181 |
| S1M10000025E11 | 2438 | SAU102437 | 5670 | SAU1c0045_orf_33p | 12695 |
| S1M10000025F03 | 2439 | SAU102297 | 5640 | SAU1c0045_orf_41p | 12704 |
| S1M10000025E05 | 2440 | SAU102200 | 5611 | SAU1c0045_orf_168p | 12665 |
| S1M10000025F05 | 2440 | SAU102201 | 5612 | SAU1c0045_orf_169p | 12666 |
| S1M10000025F08 | 2441 | SAU200685 | 5790 | SAU2c0344_orf_9p | 12801 |
| S1M10000025F09 | 2442 | SAU101907 | 5574 | SAU1c0040_orf_79p | 12442 |
| S1M10000025F10 | 2443 | SAU101571 | 5483 | SAU1c0044_orf_210p | 12585 |
| S1M10000025F12 | 2444 | SAU102200 | 5611 | SAU1c0045_orf_168p | 12665 |
| S1M10000025F12 | 2444 | SAU102201 | 5612 | SAU1c0045_orf_169p | 12666 |
| S1M10000025G04 | 2445 | SAU300617 | 5874 | SAU3c1046_orf_2p | 13056 |
| S1M10000025G06 | 2446 | SAU300617 | 5874 | SAU3c1046_orf_2p | 13056 |
| S1M10000025G10 | 2447 | SAU101869 | 5566 | SAU1c0036_orf_24p | 12321 |
| S1M10000025H05 | 2448 | SAU101907 | 5574 | SAU1c0040_orf_79p | 12442 |
| S1M10000025H06 | 2449 | SAU101907 | 5574 | SAU1c0040_orf_79p | 12442 |
| S1M10000025H07 | 2450 | SAU200752 | 5795 | SAU2c0354_orf_5p | 12809 |
| S1M10000025H07 | 2450 | SAU300975 | 5880 | SAU3c1240_orf_3p | 13075 |
| S1M10000025H10 | 2451 | SAU100590 | 5293 | SAU1c0013_orf_5p | 12121 |
| S1M10000025H10 | 2451 | SAU301268 | 5891 | SAU3c1364_orf_2p | 13102 |
| S1M10000026A02 | 2452 | SAU101907 | 5574 | SAU1c0040_orf_79p | 12442 |
| S1M10000026A04 | 2453 | SAU102340 | 5647 | SAU1c0045_orf_149p | 12660 |
| S1M10000026A05 | 2454 | SAU200934 | 5799 | SAU2c0375_orf_9p | 12842 |
| S1M10000026A06 | 2455 | SAU102059 | 5597 | SAU1c0034_orf_51p | 12286 |
| S1M10000026A07 | 2456 | SAU100970 | 5365 | SAU1c0043_orf_197p | 12529 |
| S1M10000026A08 | 2457 | SAU100266 | 5250 | SAU1c0032_orf_75p | 12256 |
| S1M10000026A09 | 2458 | SAU102452 | 5676 | SAU1c0045_orf_20p | 12674 |
| S1M10000026A09 | 2458 | SAU102453 | 5677 | SAU1c0045_orf_19p | 12669 |
| S1M10000026A10 | 2459 | SAU100970 | 5365 | SAU1c0043_orf_197p | 12529 |
| S1M10000026A11 | 2460 | SAU102259 | 5624 | SAU1c0032_orf_55p | 12245 |
| S1M10000026A11 | 2460 | SAU102260 | 5625 | SAU1c0032_orf_56p | 12246 |
| S1M10000026A11 | 2460 | SAU102261 | 5626 | SAU1c0032_orf_57p | 12247 |
| S1M10000026A11 | 2460 | SAU300868 | 5879 | #N/A | #N/A |
| S1M10000026B02 | 2461 | SAU101907 | 5574 | SAU1c0040_orf_79p | 12442 |
| S1M10000026B03 | 2462 | SAU100158 | 5238 | SAU1c0040_orf_80p | 12443 |
| S1M10000026B05 | 2463 | SAU101546 | 5475 | SAU1c0037_orf_133p | 12349 |
| S1M10000026B06 | 2464 | SAU101570 | 5482 | SAU1c0044_orf_209p | 12584 |
| S1M10000026B07 | 2465 | SAU101341 | 5424 | SAU1c0044_orf_38p | 12618 |
| S1M10000026B07 | 2465 | SAU301275 | 5892 | SAU3c1365_orf_2p | 13103 |
| S1M10000026B10 | 2466 | SAU101592 | 5490 | SAU1c0039_orf_37p | 12406 |
| S1M10000026B11 | 2467 | SAU101999 | 5585 | SAU1c0040_orf_101p | 12423 |
| S1M10000026B12 | 2468 | SAU100970 | 5365 | SAU1c0043_orf_197p | 12529 |
| S1M10000026C01 | 2469 | SAU100266 | 5250 | SAU1c0032_orf_75p | 12256 |
| S1M10000026C06 | 2470 | SAU101772 | 5526 | SAU1c0037_orf_34p | 12351 |
| S1M10000026C07 | 2471 | SAU101842 | 5557 | SAU1c0042_orf_9p | 12510 |
| S1M10000026C08 | 2472 | SAU100139 | 5234 | SAU1c0032_orf_6p | 12255 |
| S1M10000026C11 | 2473 | SAU200657 | 5789 | #N/A | #N/A |
| S1M10000026C12 | 2474 | SAU101726 | 5515 | SAU1c0016_orf_7p | 12134 |
| S1M10000026D04 | 2475 | SAU100658 | 5303 | SAU1c0038_orf_59p | 12388 |
| S1M10000026D05 | 2476 | SAU101491 | 5464 | SAU1c0025_orf_20p | 12165 |
| S1M10000026D06 | 2477 | SAU100139 | 5234 | SAU1c0032_orf_6p | 12255 |
| S1M10000026D07 | 2478 | SAU101815 | 5552 | SAU1c0032_orf_33p | 12238 |
| S1M10000026D08 | 2479 | SAU100690 | 5309 | #N/A | #N/A |
| S1M10000026D10 | 2480 | SAU203296 | 5863 | SAU2c0442_orf_18p | 12983 |
| S1M10000026D12 | 2481 | SAU100546 | 5289 | SAU1c0032_orf_2p | 12235 |
| S1M10000026E01 | 2482 | SAU101543 | 5473 | SAU1c0037_orf_130p | 12346 |
| S1M10000026E07 | 2483 | SAU102939 | 5747 | #N/A | #N/A |
| S1M10000026E09 | 2484 | SAU102001 | 5586 | SAU1c0040_orf_102p | 12424 |
| S1M10000026E09 | 2484 | SAU102002 | 5587 | SAU1c0040_orf_103p | 12425 |
| S1M10000026E10 | 2485 | SAU101869 | 5566 | SAU1c0036_orf_24p | 12321 |
| S1M10000026E11 | 2486 | SAU101791 | 5532 | SAU1c0032_orf_12p | 12216 |
| S1M10000026E12 | 2487 | SAU100964 | 5363 | SAU1c0044_orf_86p | 12641 |
| S1M10000026F01 | 2488 | SAU101784 | 5530 | SAU1c0037_orf_46p | 12355 |
| S1M10000026F03 | 2489 | SAU102200 | 5611 | SAU1c0045_orf_168p | 12665 |
| S1M10000026F03 | 2489 | SAU102201 | 5612 | SAU1c0045_orf_169p | 12666 |
| S1M10000026F04 | 2490 | SAU201571 | 5824 | SAU2c0447_orf_17p | 12997 |
| S1M10000026F05 | 2491 | SAU100139 | 5234 | SAU1c0032_orf_6p | 12255 |
| S1M10000026F06 | 2492 | SAU100414 | 5270 | SAU1c0022_orf_24p | 12148 |
| S1M10000026F07 | 2493 | SAU101869 | 5566 | SAU1c0036_orf_24p | 12321 |
| S1M10000026F08 | 2494 | SAU101756 | 5524 | SAU1c0040_orf_82p | 12445 |
| S1M10000026F09 | 2495 | SAU102939 | 5747 | #N/A | #N/A |
| S1M10000026F10 | 2496 | SAU101869 | 5566 | SAU1c0036_orf_24p | 12321 |
| S1M10000026E11 | 2497 | SAU102939 | 5747 | #N/A | #N/A |
| S1M10000026F12 | 2498 | SAU100414 | 5270 | SAU1c0022_orf_24p | 12148 |
| S1M10000026G01 | 2499 | SAU101869 | 5566 | SAU1c0036_orf_24p | 12321 |
| S1M10000026G03 | 2500 | SAU100547 | 5290 | SAU1c0032_orf_3p | 12240 |
| S1M10000026G04 | 2501 | SAU100690 | 5309 | #N/A | #N/A |
| S1M10000026G05 | 2502 | SAU101756 | 5524 | SAU1c0040_orf_82p | 12445 |
| S1M10000026G06 | 2503 | SAU101784 | 5530 | SAU1c0037_orf_46p | 12355 |
| S1M10000026G07 | 2504 | SAU100886 | 5349 | SAU1c0018_orf_16p | 12139 |
| S1M10000026G09 | 2505 | SAU100542 | 5288 | SAU1c0043_orf_210p | 12532 |
| S1M10000026G10 | 2506 | SAU100613 | 5299 | SAU1c0015_orf_14p | 12126 |
| S1M10000026G10 | 2506 | SAU102812 | 5736 | SAU1c0015_orf_15p | 12127 |
| S1M10000026G12 | 2507 | SAU101551 | 5477 | SAU1c0043_orf_67p | 12550 |
| S1M10000026H01 | 2508 | SAU101652 | 5503 | SAU1c0042_orf_123p | 12492 |
| S1M10000026H02 | 2509 | SAU102355 | 5651 | SAU1c0040_orf_40p | 12435 |
| S1M10000026H03 | 2510 | SAU101801 | 5541 | #N/A | #N/A |
| S1M10000026H04 | 2511 | SAU201810 | 5836 | SAU2c0308_orf_2p | 12769 |
| S1M10000026H04 | 2511 | SAU202174 | 5845 | SAU2c0412_orf_3p | 12895 |
| S1M10000026H04 | 2511 | SAU301148 | 5888 | #N/A | #N/A |
| S1M10000026H05 | 2512 | SAU101907 | 5574 | SAU1c0040_orf_79p | 12442 |
| S1M10000026H07 | 2513 | SAU101806 | 5546 | SAU1c0032_orf_25p | 12230 |
| S1M10000026H07 | 2513 | SAU101807 | 5547 | SAU1c0032_orf_26p | 12231 |
| S1M10000026H09 | 2514 | SAU202174 | 5845 | SAU2c0412_orf_3p | 12895 |
| S1M10000026H09 | 2514 | SAU301148 | 5888 | #N/A | #N/A |
| S1M10000026H10 | 2515 | SAU102479 | 5683 | SAU1c0039_orf_101p | 12405 |
| S1M10000027A04 | 2516 | SAU101756 | 5524 | SAU1c0040_orf_82p | 12445 |
| S1M10000027A05 | 2517 | SAU101805 | 5545 | SAU1c0032_orf_24p | 12229 |
| S1M10000027A08 | 2518 | SAU101772 | 5526 | SAU1c0037_orf_34p | 12351 |
| S1M10000027A11 | 2519 | SAU101551 | 5477 | SAU1c0043_orf_67p | 12550 |
| S1M10000027B04 | 2520 | SAU102939 | 5747 | #N/A | #N/A |
| S1M10000027B06 | 2521 | SAU100275 | 5252 | SAU1c0036_orf_15p | 12314 |
| S1M10000027B07 | 2522 | SAU100158 | 5238 | SAU1c0040_orf_80p | 12443 |
| S1M10000027B08 | 2523 | SAU101807 | 5547 | SAU1c0032_orf_26p | 12231 |
| S1M10000027B09 | 2524 | SAU102059 | 5597 | SAU1c0034_orf_51p | 12286 |
| S1M10000027B11 | 2525 | SAU101265 | 5407 | #N/A | #N/A |
| S1M10000027C02 | 2526 | SAU101327 | 5421 | SAU1c0044_orf_296p | 12612 |
| S1M10000027C04 | 2527 | SAU201236 | 5808 | SAU2c0409_orf_10p | 12891 |
| S1M10000027C05 | 2528 | SAU102117 | 5603 | SAU1c0027_orf_6p | 12181 |
| S1M10000027C06 | 2529 | SAU100114 | 5228 | SAU1c0043_orf_225p | 12535 |
| S1M10000027C08 | 2530 | SAU101807 | 5547 | SAU1c0032_orf_26p | 12231 |
| S1M10000027C09 | 2531 | SAU101545 | 5474 | SAU1c0037_orf_132p | 12348 |
| S1M10000027D02 | 2532 | SAU101652 | 5503 | SAU1c0042_orf_123p | 12492 |
| S1M10000027D02 | 2532 | SAU101653 | 5504 | SAU1c0042_orf_124p | 12493 |
| S1M10000027D03 | 2533 | SAU100300 | 5253 | SAU1c0040_orf_90p | 12451 |
| S1M10000027D05 | 2534 | SAU101554 | 5478 | SAU1c0043_orf_70p | 12551 |
| S1M10000027D06 | 2535 | SAU202708 | 5849 | SAU2c0385_orf_1p | 12855 |
| S1M10000027D08 | 2536 | SAU100714 | 5312 | SAU1c0044_orf_74p | 12635 |
| S1M10000027D09 | 2537 | SAU203524 | 5864 | SAU2c0435_orf_1p | 12957 |
| S1M10000027D10 | 2538 | SAU102283 | 5634 | SAU1c0006_orf_1p | 12119 |
| S1M10000027D11 | 2539 | SAU101996 | 5584 | SAU1c0040_orf_99p | 12456 |
| S1M10000027E05 | 2540 | SAU200916 | 5797 | SAU2c0373_orf_4p | 12838 |
| S1M10000027E05 | 2540 | SAU301620 | 5899 | SAU3c1478_orf_2p | 13140 |
| S1M10000027E06 | 2541 | SAU100690 | 5309 | #N/A | #N/A |
| S1M10000027E07 | 2542 | SAU100547 | 5290 | SAU1c0032_orf_3p | 12240 |
| S1M10000027E08 | 2543 | SAU201571 | 5824 | SAU2c0447_orf_17p | 12997 |
| S1M10000027E09 | 2544 | SAU101807 | 5547 | SAU1c0032_orf_26p | 12231 |
| S1M10000027E11 | 2545 | SAU101551 | 5477 | SAU1c0043_orf_67p | 12550 |
| S1M10000027F01 | 2546 | SAU103038 | 5757 | #N/A | 4N/A |
| S1M10000027F02 | 2547 | SAU101491 | 5464 | SAU1c0025_orf_20p | 12165 |
| S1M10000027F05 | 2548 | SAU100882 | 5347 | SAU1c0038_orf_35p | 12374 |
| S1M10000027F06 | 2549 | SAU100690 | 5309 | #N/A | #N/A |
| S1M10000027F08 | 2550 | SAU200006 | 5770 | SAU2c0157_orf_1p | 12723 |
| S1M10000027F09 | 2551 | SAU100858 | 5341 | SAU1c0038_orf_86p | 12401 |
| S1M10000027G03 | 2552 | SAU101756 | 5524 | SAU1c0040_orf_82p | 12445 |
| S1M10000027G04 | 2553 | SAU101777 | 5527 | SAU1c0037_orf_39p | 12352 |
| S1M10000027G05 | 2554 | SAU102526 | 5692 | SAU1c0045_orf_299p | 12691 |
| S1M10000027G06 | 2555 | SAU202708 | 5849 | SAU2c0385_orf_1p | 12855 |
| S1M10000027G07 | 2556 | SAU102265 | 5629 | SAU1c0032_orf_61p | 12251 |
| S1M10000027G09 | 2557 | SAU101807 | 5547 | SAU1c0032_orf_26p | 12231 |
| S1M10000027G11 | 2558 | SAU102533 | 5695 | #N/A | #N/A |
| S1M10000027G11 | 2558 | SAU102534 | 5696 | #N/A | #N/A |
| S1M10000027H02 | 2559 | SAU102059 | 5597 | SAU1c0034_orf_51p | 12286 |
| S1M10000027H04 | 2560 | SAU101777 | 5527 | SAU1c0037_orf_39p | 12352 |
| S1M10000027H05 | 2561 | SAU102526 | 5692 | SAU1c0045_orf_299p | 12691 |
| S1M10000027H06 | 2562 | SAU100690 | 5309 | #N/A | #N/A |
| S1M10000027H07 | 2563 | SAU100542 | 5288 | SAU1c0043_orf_210p | 12532 |
| S1M10000027H08 | 2564 | SAU201571 | 5824 | SAU2c0447_orf_17p | 12997 |
| S1M10000027H09 | 2565 | SAU101382 | 5437 | SAU1c0022_orf_19p | 12146 |
| S1M10000027H10 | 2566 | SAU100158 | 5238 | SAU1c0040_orf_80p | 12443 |
| S1M10000027H11 | 2567 | SAU102533 | 5695 | #N/A | #N/A |
| S1M10000027H11 | 2567 | SAU102534 | 5696 | #N/A | #N/A |
| S1M10000028A02 | 2568 | SAU101085 | 5378 | SAU1c0034_orf_42p | 1284 |
| S1M10000028A02 | 2568 | SAU101086 | 5379 | SAU1c0034_orf_43p | 1285 |
| S1M10000028A04 | 2569 | SAU101028 | 5370 | SAU1c0043_orf_7p | 12552 |
| S1M10000028A06 | 2570 | SAU100478 | 5277 | SAU1c0044_orf_265p | 12605 |
| S1M10000028A06 | 2570 | SAU100996 | 5366 | SAU1c0044_orf_266p | 12606 |
| S1M10000028A08 | 2571 | SAU102054 | 5596 | SAU1c0039_orf_74p | 12417 |
| S1M10000028B01 | 2572 | SAU101085 | 5378 | SAU1c0034_orf_42p | 12284 |
| S1M10000028B01 | 2572 | SAU101086 | 5379 | SAU1c0034_orf_43p | 12285 |
| S1M10000028B02 | 2573 | SAU102059 | 5597 | SAU1c0034_orf_51p | 12286 |
| S1M10000028B02 | 2573 | SAU301465 | 5896 | SAU3c1429_orf_4p | 13121 |
| S1M10000028B03 | 2574 | SAU100887 | 5350 | SAU1c0018_orf_15p | 12138 |
| S1M10000028B04 | 2575 | SAU102764 | 5734 | SAU1c0044_orf_56p | 12625 |
| S1M10000028B05 | 2576 | SAU101869 | 5566 | SAU1c0036_orf_24p | 12321 |
| S1M10000028B06 | 2577 | SAU201558 | 5823 | SAU2c0434_orf_5p | 12954 |
| S1M10000028B08 | 2578 | SAU100158 | 5238 | SAU1c0040_orf_80p | 12443 |
| S1M10000028B09 | 2579 | SAU100158 | 5238 | SAU1c0040_orf_80p | 12443 |
| S1M10000028C02 | 2580 | SAU203296 | 5863 | SAU2c0442_orf_18p | 12983 |
| S1M10000028C04 | 2581 | SAU101381 | 5436 | SAU1c0022_orf_18p | 12145 |
| S1M10000028C05 | 2582 | SAU100313 | 5259 | SAU1c0045_orf_153p | 12661 |
| S1M10000028C05 | 2582 | SAU100359 | 5264 | SAU1c0032_orf_35p | 12239 |
| S1M10000028C05 | 2582 | SAU200297 | 5778 | SAU2c0274_orf_2p | 12739 |
| S1M10000028C06 | 2583 | SAU103226 | 5768 | SAU1c0045_orf_95p | 12713 |
| S1M10000028C08 | 2584 | SAU101752 | 5522 | SAU1c0040_orf_85p | 12447 |
| S1M10000028D03 | 2585 | SAU301898 | 5904 | SAU3c1079_orf_1p | 13057 |
| S1M10000028D04 | 2586 | SAU101381 | 5436 | SAU1c0022_orf_18p | 12145 |
| S1M10000028D06 | 2587 | SAU200006 | 5770 | SAU2c0157_orf_1p | 12723 |
| S1M10000028D07 | 2588 | SAU101271 | 5411 | SAU1c0037_orf_90p | 12366 |
| S1M10000028D08 | 2589 | SAU100858 | 5341 | SAU1c0038_orf_86p | 12401 |
| S1M10000028D09 | 2590 | SAU100158 | 5238 | SAU1c0040_orf_80p | 12443 |
| S1M10000028E01 | 2591 | SAU100062 | 5225 | SAU1c0035_orf_98p | 12309 |
| S1M10000028E01 | 2591 | SAU100231 | 5245 | #N/A | #N/A |
| S1M10000028E03 | 2592 | SAU100770 | 5324 | #N/A | #N/A |
| S1M10000028E08 | 2593 | SAU101865 | 5563 | SAU1c0036_orf_20p | 12318 |
| S1M10000028F01 | 2594 | SAU101085 | 5378 | SAU1c0034_orf_42p | 12284 |
| S1M10000028F01 | 2594 | SAU101086 | 5379 | SAU1c0034_orf_43p | 12285 |
| S1M10000028F03 | 2595 | SAU100414 | 5270 | SAU1c0022_orf_24p | 12148 |
| S1M10000028F04 | 2596 | SAU100301 | 5254 | SAU1c0040_orf_91p | 12452 |
| S1M10000028F04 | 2596 | SAU100302 | 5255 | SAU1c0040_orf_92p | 12453 |
| S1M10000028F05 | 2597 | SAU100301 | 5254 | SAU1c0040_orf_91p | 12452 |
| S1M10000028F05 | 2597 | SAU100302 | 5255 | SAU1c0040_orf_92p | 12453 |
| S1M10000028F06 | 2598 | SAU100432 | 5271 | SAU1c0040_orf_88p | 12450 |
| S1M10000028F06 | 2598 | SAU202756 | 5852 | SAU2c0470_orf_1p | 13027 |
| S1M10000028F07 | 2599 | SAU101006 | 5367 | SAU1c0028_orf_59p | 12190 |
| S1M10000028G01 | 2600 | SAU102554 | 5699 | SAU1c0045_orf_209p | 12673 |
| S1M10000028G02 | 2601 | SAU201236 | 5808 | SAU2c0409_orf_10p | 12891 |
| S1M10000028G02 | 2601 | SAU300338 | 5871 | #N/A | #N/A |
| S1M10000028G03 | 2602 | SAU101231 | 5399 | SAU1c0035_orf_6p | 12303 |
| S1M10000028G04 | 2603 | SAU200916 | 5797 | SAU2c0373_orf_4p | 12838 |
| S1M10000028G04 | 2603 | SAU301620 | 5899 | SAU3c1478_orf_2p | 13140 |
| S1M10000028G05 | 2604 | SAU100690 | 5309 | #N/A | #N/A |
| S1M10000028G06 | 2605 | SAU101865 | 5563 | SAU1c0036_orf_20p | 12318 |
| S1M10000028G08 | 2606 | SAU101341 | 5424 | SAU1c0044_orf_38p | 12618 |
| S1M10000028G08 | 2606 | SAU301275 | 5892 | SAU3c1365_orf_2p | 13103 |
| S1M10000028H03 | 2607 | SAU101815 | 5552 | SAU1c0032_orf_33p | 12238 |
| S1M10000028H04 | 2608 | SAU103038 | 5757 | #N/A | #N/A |
| S1M10000028H05 | 2609 | SAU101869 | 5566 | SAU1c0036_orf_24p | 12321 |
| S1M10000029A02 | 2610 | SAU100887 | 5350 | SAU1c0018_orf_15p | 12138 |
| S1M10000029A04 | 2611 | SAU100489 | 5278 | SAU1c0044_orf_133p | 12566 |
| S1M10000029A04 | 2611 | SAU100557 | 5291 | SAU1c0044_orf_132p | 12565 |
| S1M10000029A09 | 2612 | SAU101495 | 5467 | SAU1c0037_orf_65p | 12360 |
| S1M10000029A10 | 2613 | SAU100414 | 5270 | SAU1c0022_orf_24p | 12148 |
| S1M10000029A11 | 2614 | SAU101868 | 5565 | SAU1c0036_orf_23p | 12320 |
| S1M10000029A12 | 2615 | SAU100865 | 5343 | SAU1c0044_orf_99p | 12648 |
| S1M10000029B02 | 2616 | SAU200928 | 5798 | SAU2c0365_orf_5p | 12815 |
| S1M10000029B03 | 2617 | SAU201225 | 5807 | SAU2c0412_orf_5p | 12896 |
| S1M10000029B04 | 2618 | SAU201621 | 5828 | SAU2c0437_orf_4p | 12966 |
| S1M10000029B05 | 2619 | SAU100355 | 5263 | SAU1c0023_orf_6p | 12155 |
| S1M10000029B06 | 2620 | SAU201571 | 5824 | SAU2c0447_orf_17p | 12997 |
| S1M10000029B08 | 2621 | SAU101360 | 5431 | SAU1c0044_orf_109p | 12555 |
| S1M10000029B10 | 2622 | SAU101891 | 5571 | SAU1c0034_orf_30p | 12281 |
| S1M10000029C02 | 2623 | SAU101271 | 5411 | SAU1c0037_orf_90p | 12366 |
| S1M10000029C03 | 2624 | SAU100690 | 5309 | #N/A | #N/A |
| S1M10000029C05 | 2625 | SAU200928 | 5798 | SAU2c0365_orf_5p | 12815 |
| S1M10000029C07 | 2626 | SAU102222 | 5613 | SAU1c0043_orf_12p | 12511 |
| S1M10000029C09 | 2627 | SAU101495 | 5467 | SAU1c0037_orf_65p | 12360 |
| S1M10000029C10 | 2628 | SAU101995 | 5583 | SAU1c0040_orf_98p | 12455 |
| S1M10000029C12 | 2629 | SAU100859 | 5342 | SAU1c0038_orf_87p | 12402 |
| S1M10000029D02 | 2630 | SAU101400 | 5444 | SAU1c0036_orf_35p | 12326 |
| S1M10000029D05 | 2631 | SAU100887 | 5350 | SAU1c0018_orf_15p | 12138 |
| S1M10000029D09 | 2632 | SAU101495 | 5467 | SAU1c0037_orf_65p | 12360 |
| S1M10000029D10 | 2633 | SAU101891 | 5571 | SAU1c0034_orf_30p | 12281 |
| S1M10000029D12 | 2634 | SAU100056 | 5223 | SAU1c0044_orf_176p | 12577 |
| S1M10000029E02 | 2635 | SAU101400 | 5444 | SAU1c0036_orf_35p | 12326 |
| S1M10000029E05 | 2636 | SAU100522 | 5284 | SAU1c0044_orf_249p | 12599 |
| S1M10000029E10 | 2637 | SAU101271 | 5411 | SAU1c0037_orf_90p | 12366 |
| S1M10000029E11 | 2638 | SAU101271 | 5411 | SAU1c0037_orf_90p | 12366 |
| S1M10000029F01 | 2639 | SAU101803 | 5543 | SAU1c0032_orf_23p | 1228 |
| S1M10000029F01 | 2639 | SAU101804 | 5544 | #N/A | #N/A |
| S1M10000029F02 | 2640 | SAU101271 | 5411 | SAU1c0037_orf_90p | 12366 |
| S1M10000029F02 | 2640 | SAU101286 | 5413 | SAU1c0034_orf_67p | 12292 |
| S1M10000029F04 | 2641 | SAU102639 | 5724 | #N/A | #N/A |
| S1M10000029F09 | 2642 | SAU100793 | 5329 | SAU1c0028_orf_52p | 12188 |
| S1M10000029F09 | 2642 | SAU301433 | 5895 | SAU3c1420_orf_2p | 13118 |
| S1M10000029F10 | 2643 | SAU102621 | 5719 | SAU1c0041_orf_63p | 12480 |
| S1M10000029F11 | 2644 | SAU102883 | 5741 | SAU1c0045_orf_38p | 12702 |
| S1M10000029F12 | 2645 | SAU102603 | 5709 | SAU1c0041_orf_48p | 12469 |
| S1M10000029F12 | 2645 | SAU102609 | 5713 | SAU1c0041_orf_52p | 12473 |
| S1M10000029G01 | 2646 | SAU101752 | 5522 | SAU1c0040_orf_85p | 12447 |
| S1M10000029002 | 2647 | SAU101622 | 5496 | SAU1c0040_orf_27p | 12430 |
| S1M10000029G03 | 2648 | SAU201571 | 5824 | SAU2c0447_orf_17p | 12997 |
| S1M10000029G05 | 2649 | SAU101156 | 5386 | SAU1c0036_orf_12p | 12311 |
| S1M10000029G07 | 2650 | SAU101622 | 5496 | SAU1c0040_orf_27p | 12430 |
| S1M10000029G08 | 2651 | SAU101365 | 5432 | SAU1c0044_orf_112p | 12556 |
| S1M10000029G12 | 2652 | SAU101270 | 5410 | SAU1c0037_orf_89p | 12365 |
| S1M10000029H01 | 2653 | SAU100414 | 5270 | SAU1c0022_orf_24p | 12148 |
| S1M10000029H05 | 2654 | SAU102613 | 5715 | SAU1c0041_orf_55p | 12475 |
| S1M10000029H06 | 2655 | SAU200928 | 5798 | SAU2c0365_orf_5p | 12815 |
| S1M10000029H08 | 2656 | SAU101271 | 5411 | SAU1c0037_orf_90p | 12366 |
| S1M10000029H09 | 2657 | SAU101365 | 5432 | SAU1c0044_orf_112p | 12556 |
| S1M10000029H10 | 2658 | SAU101271 | 5411 | SAU1c0037_orf_90p | 12366 |
| S1M10000030A02 | 2659 | SAU101543 | 5473 | SAU1c0037_orf_130p | 12346 |
| S1M10000030A05 | 2660 | SAU101491 | 5464 | SAU1c0025_orf_20p | 12165 |
| S1M10000030A09 | 2661 | SAU101242 | 5404 | SAU1c0044_orf_18p | 12578 |
| S1M10000030A10 | 2662 | SAU101092 | 5381 | SAU1c0028_orf_9p | 12192 |
| S1M10000030A10 | 2662 | SAU202882 | 5855 | SAU2c0381_orf_3p | 12848 |
| S1M10000030A11 | 2663 | SAU100414 | 5270 | SAU1c0022_orf_24p | 12148 |
| S1M10000030B02 | 2664 | SAU101573 | 5485 | SAU1c0044_orf_212p | 12587 |
| S1M10000030B05 | 2665 | SAU100275 | 5252 | SAU1c0036_orf_15p | 12314 |
| S1M10000030B07 | 2666 | SAU101180 | 5389 | SAU1c0045_orf_126p | 12656 |
| S1M10000030B09 | 2667 | SAU301898 | 5904 | SAU3c1079_orf_1p | 13057 |
| S1M10000030C02 | 2668 | SAU102531 | 5694 | SAU1c0045_orf_186p | 12667 |
| S1M10000030C03 | 2669 | SAU102629 | 5720 | SAU1c0041_orf_71p | 12481 |
| S1M10000030C04 | 2670 | SAU101999 | 5585 | SAU1c0040_orf_101p | 12423 |
| S1M10000030C05 | 2671 | SAU101999 | 5585 | SAU1c0040_orf_101p | 12423 |
| S1M10000030C08 | 2672 | SAU101175 | 5388 | SAU1c0031_orf_1p | 12213 |
| S1M10000030C09 | 2673 | SAU101752 | 5522 | SAU1c0040_orf_85p | 12447 |
| S1M10000030C10 | 2674 | SAU301592 | 5898 | SAU3c1467_orf_2p | 13137 |
| S1M10000030C12 | 2675 | SAU100961 | 5360 | SAU1c0044_orf_83p | 12638 |
| S1M10000030C12 | 2675 | SAU100962 | 5361 | SAU1c0044_orf_84p | 12639 |
| S1M10000030D01 | 2676 | SAU101495 | 5467 | SAU1c0037_orf_65p | 12360 |
| S1M10000030D02 | 2677 | SAU101573 | 5485 | SAU1c0044_orf_212p | 12587 |
| S1M10000030D03 | 2678 | SAU100731 | 5313 | SAU1c0044_orf_252p | 12601 |
| S1M10000030D05 | 2679 | SAU102222 | 5613 | SAU1c0043_orf_12p | 12511 |
| S1M10000030D06 | 2680 | SAU102392 | 5658 | SAU1c0033_orf_40p | 12270 |
| S1M10000030D06 | 2680 | SAU201541 | 5822 | SAU2c0431_orf_14p | 12942 |
| S1M10000030D07 | 2681 | SAU102392 | 5658 | SAU1c0033_orf_40p | 12270 |
| S1M10000030D07 | 2681 | SAU201541 | 5822 | SAU2c0431_orf_14p | 12942 |
| S1M10000029F09 | 2642 | SAU100793 | 5329 | SAU1c0028_orf_52p | 12188 |
| S1M10000029F09 | 2642 | SAU301433 | 5895 | SAU3c1420_orf_2p | 13118 |
| S1M10000029F10 | 2643 | SAU102621 | 5719 | SAU1c0041_orf_63p | 12480 |
| S1M10000029F11 | 2644 | SAU102883 | 5741 | SAU1c0045_orf_38p | 12702 |
| S1M10000029F12 | 2645 | SAU102603 | 5709 | SAU1c0041_orf_48p | 12469 |
| S1M10000029F12 | 2645 | SAU102609 | 5713 | SAU1c0041_orf_52p | 12473 |
| S1M10000029G01 | 2646 | SAU101752 | 5522 | SAU1c0040_orf_85p | 12447 |
| S1M10000029G02 | 2647 | SAU101622 | 5496 | SAU1c0040_orf_27p | 12430 |
| S1M10000029G03 | 2648 | SAU201571 | 5824 | SAU2c0447_orf_17p | 12997 |
| S1M10000029G05 | 2649 | SAU101156 | 5386 | SAU1c0036_orf_12p | 12311 |
| S1M10000029G07 | 2650 | SAU101622 | 5496 | SAU1c0040_orf_27p | 12430 |
| S1M10000029G08 | 2651 | SAU101365 | 5432 | SAU1c0044_orf_112p | 12556 |
| S1M10000029G12 | 2652 | SAU101270 | 5410 | SAU1c0037_orf_89p | 12365 |
| S1M10000029H01 | 2653 | SAU100414 | 5270 | SAU1c0022_orf_24p | 12148 |
| S1M10000029H05 | 2654 | SAU102613 | 5715 | SAU1c0041_orf_55p | 12475 |
| S1M10000029H06 | 2655 | SAU200928 | 5798 | SAU2c0365_orf_5p | 12815 |
| S1M10000029H08 | 2656 | SAU101271 | 5411 | SAU1c0037_orf_90p | 12366 |
| S1M10000029H09 | 2657 | SAU101365 | 5432 | SAU1c0044_orf_112p | 12556 |
| S1M10000029H10 | 2658 | SAU101271 | 5411 | SAU1c0037_orf_90p | 12366 |
| S1M10000030A02 | 2659 | SAU101543 | 5473 | SAU1c0037_orf_130p | 12346 |
| S1M10000030A05 | 2660 | SAU101491 | 5464 | SAU1c0025_orf_20p | 12165 |
| S1M10000030A09 | 2661 | SAU101242 | 5404 | SAU1c0044_orf_18p | 12578 |
| S1M10000030A10 | 2662 | SAU101092 | 5381 | SAU1c0028_orf_9p | 12192 |
| S1M10000030A10 | 2662 | SAU202882 | 5855 | SAU2c0381_orf_3p | 12848 |
| S1M10000030A11 | 2663 | SAU100414 | 5270 | SAU1c0022_orf_24p | 12148 |
| S1M10000030B02 | 2664 | SAU101573 | 5485 | SAU1c0044_orf_212p | 12587 |
| S1M10000030B05 | 2665 | SAU100275 | 5252 | SAU1c0036_orf_15p | 12314 |
| S1M10000030B07 | 2666 | SAU101180 | 5389 | SAU1c0045_orf_126p | 12656 |
| S1M10000030B09 | 2667 | SAU301898 | 5904 | SAU3c1079_orf_1p | 13057 |
| S1M10000030C02 | 2668 | SAU102531 | 5694 | SAU1c0045_orf_186p | 12667 |
| S1M10000030C03 | 2669 | SAU102629 | 5720 | SAU1c0041_orf_71p | 12481 |
| S1M10000030C04 | 2670 | SAU101999 | 5585 | SAU1c0040_orf_101p | 12423 |
| S1M10000030C05 | 2671 | SAU101999 | 5585 | SAU1c0040_orf_101p | 12423 |
| S1M10000030C08 | 2672 | SAU101175 | 5388 | SAU1c0031_orf_1p | 12213 |
| S1M10000030C09 | 2673 | SAU101752 | 5522 | SAU1c0040_orf_85p | 12447 |
| S1M10000030C10 | 2674 | SAU301592 | 5898 | SAU3c1467_orf_2p | 13137 |
| S1M10000030C12 | 2675 | SAU100961 | 5360 | SAU1c0044_orf_83p | 12638 |
| S1M10000030C12 | 2675 | SAU100962 | 5361 | SAU1c0044_orf_84p | 12639 |
| S1M10000030D01 | 2676 | SAU101495 | 5467 | SAU1c0037_orf_65p | 12360 |
| S1M10000030D02 | 2677 | SAU101573 | 5485 | SAU1c0044_orf_212p | 12587 |
| S1M10000030D03 | 2678 | SAU100731 | 5313 | SAU1c0044_orf_252p | 12601 |
| S1M10000030D05 | 2679 | SAU102222 | 5613 | SAU1c0043_orf_12p | 12511 |
| S1M10000030D06 | 2680 | SAU102392 | 5658 | SAU1c0033_orf_40p | 12270 |
| S1M10000030D06 | 2680 | SAU201541 | 5822 | SAU2c0431_orf_14p | 12942 |
| S1M10000030D07 | 2681 | SAU102392 | 5658 | SAU1c0033_orf_40p | 12270 |
| S1M10000030D07 | 2681 | SAU201541 | 5822 | SAU2c0431_orf_14p | 12942 |
| S1M10000030D09 | 2682 | SAU101271 | 5411 | SAU1c0037_orf_90p | 12366 |
| S1M10000030D10 | 2683 | SAU100313 | 5259 | SAU1c0045_orf_153p | 12661 |
| S1M10000030D10 | 2683 | SAU100359 | 5264 | SAU1c0032_orf_35p | 12239 |
| S1M10000030D11 | 2684 | SAU100414 | 5270 | SAU1c0022_orf_24p | 12148 |
| S1M10000030E02 | 2685 | SAU100731 | 5313 | SAU1c0044_orf_252p | 12601 |
| S1M10000030E06 | 2686 | SAU102909 | 5743 | SAU1c0036_orf_16p | 12315 |
| S1M10000030E07 | 2687 | SAU102939 | 5747 | #N/A | #N/A |
| S1M10000030E11 | 2688 | SAU101790 | 5531 | SAU1c0032_orf_11p | 12215 |
| S1M10000030E12 | 2689 | SAU100300 | 5253 | SAU1c0040_orf_90p | 12451 |
| S1M10000030F01 | 2690 | SAU100731 | 5313 | SAU1c0044_orf_252p | 12601 |
| S1M10000030F07 | 2691 | SAU102939 | 5747 | #N/A | #N/A |
| S1M10000030F08 | 2692 | SAU101800 | 5540 | SAU1c0032_orf_20p | 12225 |
| S1M10000030F08 | 2692 | SAU101801 | 5541 | #N/A | #N/A |
| S1M10000030F09 | 2693 | SAU101266 | 5408 | SAU1c0042_orf_117p | 12490 |
| S1M10000030F10 | 2694 | SAU102453 | 5677 | SAU1c0045_orf_19p | 12669 |
| S1M10000030G03 | 2695 | SAU101752 | 5522 | SAU1c0040_orf_85p | 12447 |
| S1M10000030G05 | 2696 | SAU102246 | 5619 | SAU1c0043_orf_30p | 12542 |
| S1M10000030G05 | 2696 | SAU102247 | 5620 | SAU1c0043_orf_31p | 12543 |
| S1M10000030G07 | 2697 | SAU102602 | 5708 | SAU1c0032_orf_5p | 12249 |
| S1M10000030G08 | 2698 | SAU100546 | 5289 | SAU1c0032_orf_2p | 12235 |
| S1M10000030G09 | 2699 | SAU101752 | 5522 | SAU1c0040_orf_85p | 12447 |
| S1M10000030G10 | 2700 | SAU102453 | 5677 | SAU1c0045_orf_19p | 12669 |
| S1M10000030G11 | 2701 | SAU101529 | 5471 | SAU1c0043_orf_39p | 12544 |
| S1M10000030G12 | 2702 | SAU201197 | 5806 | SAU2c0429_orf_2p | 12938 |
| S1M10000030H01 | 2703 | SAU200928 | 5798 | SAU2c0365_orf_5p | 12815 |
| S1M10000030H02 | 2704 | SAU200392 | 5780 | SAU2c0298_orf_3p | 12755 |
| S1M10000030H03 | 2705 | SAU102162 | 5609 | SAU1c0041_orf_27p | 12462 |
| S1M10000030H05 | 2706 | SAU102380 | 5654 | SAU1c0033_orf_29p | 12265 |
| S1M10000030H07 | 2707 | SAU100123 | 5230 | SAU1c0043_orf_189p | 12526 |
| S1M10000030H07 | 2707 | SAU102001 | 5586 | SAU1c0040_orf_102p | 12424 |
| S1M10000030H07 | 2707 | SAU103159 | 5762 | SAU1c0045_orf_204p | 12670 |
| S1M10000030H07 | 2707 | SAU201827 | 5837 | SAU2c0449_orf_21p | 13002 |
| S1M10000030H09 | 2708 | SAU100964 | 5363 | SAU1c0044_orf_86p | 12641 |
| S1M10000031A03 | 2709 | SAU100546 | 5289 | SAU1c0032_orf_2p | 12235 |
| S1M10000031A08 | 2710 | SAU101641 | 5501 | SAU1c0029_orf_12p | 12193 |
| S1M10000031A10 | 2711 | SAU102242 | 5618 | SAU1c0043_orf_26p | 12540 |
| S1M10000031B01 | 2712 | SAU101791 | 5532 | SAU1c0032_orf_12p | 12216 |
| S1M10000031B02 | 2713 | SAU102602 | 5708 | SAU1c0032_orf_5p | 12249 |
| S1M10000031B04 | 2714 | SAU200928 | 5798 | SAU2c0365_orf_5p | 12815 |
| S1M10000031B11 | 2715 | SAU101262 | 5406 | SAU1c0042_orf_113p | 12488 |
| S1M10000031B12 | 2716 | SAU101360 | 5431 | SAU1c0044_orf_109p | 12555 |
| S1M10000031C04 | 2717 | SAU100062 | 5225 | SAU1c0035_orf_98p | 12309 |
| S1M10000031C04 | 2717 | SAU100231 | 5245 | #N/A | #N/A |
| S1M10000031C07 | 2718 | SAU102059 | 5597 | SAU1c0034_orf_51p | 12286 |
| S1M10000031C09 | 2719 | SAU102117 | 5603 | SAU1c0027_orf_6p | 12181 |
| S1M10000031C11 | 2720 | SAU102935 | 5745 | #N/A | #N/A |
| S1M10000031D06 | 2721 | SAU201197 | 5806 | SAU2c0429_orf_2p | 12938 |
| S1M10000031D07 | 2722 | SAU101543 | 5473 | SAU1c0037_orf_130p | 12346 |
| S1M10000031D08 | 2723 | SAU101891 | 5571 | SAU1c0034_orf_30p | 12281 |
| S1M10000031D09 | 2724 | SAU102453 | 5677 | SAU1c0045_orf_19p | 12669 |
| S1M10000031E02 | 2725 | SAU101350 | 5429 | SAU1c0042_orf_109p | 12487 |
| S1M10000031E03 | 2726 | SAU101267 | 5409 | SAU1c0037_orf_86p | 12364 |
| S1M10000031E03 | 2726 | SAU300719 | 5876 | SAU3c1108_orf_3p | 13059 |
| S1M10000031E04 | 2727 | SAU101752 | 5522 | SAU1c0040_orf_85p | 12447 |
| S1M10000031E07 | 2728 | SAU102449 | 5674 | SAU1c0045_orf_22p | 12677 |
| S1M10000031E08 | 2729 | SAU100158 | 5238 | SAU1c0040_orf_80p | 12443 |
| S1M10000031E10 | 2730 | SAU102433 | 5668 | SAU1c0045_orf_37p | 12701 |
| S1M10000031E12 | 2731 | SAU101400 | 5444 | SAU1c0036_orf_35p | 12326 |
| S1M10000031F02 | 2732 | SAU101800 | 5540 | SAU1c0032_orf_20p | 12225 |
| S1M10000031F02 | 2732 | SAU101801 | 5541 | #N/A | 4N/A |
| S1M10000031F03 | 2733 | SAU101791 | 5532 | SAU1c0032_orf_12p | 12216 |
| S1M10000031F04 | 2734 | SAU101571 | 5483 | SAU1c0044_orf_210p | 12585 |
| S1M10000031F04 | 2734 | SAU101572 | 5484 | SAU1c0044_orf_211p | 12586 |
| S1M10000031F05 | 2735 | SAU101907 | 5574 | SAU1c0040_orf_79p | 12442 |
| S1M10000031F08 | 2736 | SAU101869 | 5566 | SAU1c0036_orf_24p | 12321 |
| S1M10000031F10 | 2737 | SAU102593 | 5704 | SAU1c0041_orf_39p | 12463 |
| S1M10000031F11 | 2738 | SAU102469 | 5679 | SAU1c0026_orf_25p | 12172 |
| S1M10000031F12 | 2739 | SAU102593 | 5704 | SAU1c0041_orf_39p | 12463 |
| S1M10000031002 | 2740 | SAU101797 | 5537 | SAU1c0032_orf_17p | 12221 |
| S1M10000031003 | 2741 | SAU101679 | 5509 | SAU1c0044_orf_222p | 12593 |
| S1M10000031G04 | 2742 | SAU103198 | 5766 | #N/A | #N/A |
| S1M10000031006 | 2743 | SAU101907 | 5574 | SAU1c0040_orf_79p | 12442 |
| S1M10000031G09 | 2744 | SAU201571 | 5824 | SAU2c0447_orf_17p | 12997 |
| S1M10000031G10 | 2745 | SAU100077 | 5226 | SAU1c0043_orf_178p | 12520 |
| S1M10000031G11 | 2746 | SAU100118 | 5229 | SAU1c0015_orf_13p | 12125 |
| S1M10000031H01 | 2747 | SAU103144 | 5761 | SAU1c0045_orf_15p | 12663 |
| S1M10000031H02 | 2748 | SAU100886 | 5349 | SAU1c0018_orf_16p | 12139 |
| S1M10000031H06 | 2749 | SAU100690 | 5309 | #N/A | #N/A |
| S1M10000031H09 | 2750 | SAU201743 | 5831 | #N/A | #N/A |
| S1M10000031H11 | 2751 | SAU100077 | 5226 | SAU1c0043_orf_178p | 12520 |
| S1M10000032A03 | 2752 | SAU202039 | 5843 | SAU2c0452_orf_20p | 13009 |
| S1M10000032A05 | 2753 | SAU100275 | 5252 | SAU1c0036_orf_15p | 12314 |
| S1M10000032A06 | 2754 | SAU100610 | 5298 | SAU1c0034_orf_71p | 12294 |
| S1M10000032A07 | 2755 | SAU102059 | 5597 | SAU1c0034_orf_51p | 12286 |
| S1M10000032A08 | 2756 | SAU102142 | 5606 | SAU1c0041_orf_13p | 12457 |
| S1M10000032A08 | 2756 | SAU102143 | 5607 | SAU1c0041_orf_14p | 12458 |
| S1M10000032A10 | 2757 | SAU101777 | 5527 | SAU1c0037_orf_39p | 12352 |
| S1M10000032B01 | 2758 | SAU301898 | 5904 | SAU3c1079_orf_1p | 13057 |
| S1M10000032B05 | 2759 | SAU102607 | 5712 | SAU1c0041_orf_51p | 12472 |
| S1M10000032B05 | 2759 | SAU102944 | 5749 | SAU1c0041_orf_47p | 12468 |
| S1M10000032B07 | 2760 | SAU100157 | 5237 | SAU1c0040_orf_81p | 12444 |
| S1M10000032B08 | 2761 | SAU100175 | 5240 | SAU1c0044_orf_204p | 12582 |
| S1M10000032B11 | 2762 | SAU100944 | 5357 | SAU1c0042_orf_5p | 12505 |
| S1M10000032B12 | 2763 | SAU102117 | 5603 | SAU1c0027_orf_6p | 12181 |
| S1M10000032C01 | 2764 | SAU101907 | 5574 | SAU1c0040_orf_79p | 12442 |
| S1M10000032C03 | 2765 | SAU102241 | 5617 | SAU1c0043_orf_25p | 12539 |
| S1M10000032C04 | 2766 | SAU102241 | 5617 | SAU1c0043_orf_25p | 12539 |
| S1M10000032C05 | 2767 | SAU101632 | 5499 | SAU1c0039_orf_3p | 12407 |
| S1M10000032C09 | 2768 | SAU101907 | 5574 | SAU1c0040_orf_79p | 12442 |
| S1M10000032C10 | 2769 | SAU201615 | 5826 | SAU2c0440_orf_10p | 12972 |
| S1M10000032C11 | 2770 | SAU102863 | 5737 | #N/A | #N/A |
| S1M10000032C12 | 2771 | SAU102863 | 5737 | #N/A | #N/A |
| S1M10000032D03 | 2772 | SAU100613 | 5299 | SAU1c0015_orf_14p | 12126 |
| S1M10000032D06 | 2773 | SAU101652 | 5503 | SAU1c0042_orf_123p | 12492 |
| S1M10000032D07 | 2774 | SAU200468 | 5781 | SAU2c0429_orf_19p | 12937 |
| S1M10000032D09 | 2775 | SAU100128 | 5231 | #N/A | #N/A |
| S1M10000032D09 | 2775 | SAU101549 | 5476 | SAU1c0043_orf_64p | 12549 |
| S1M10000032D09 | 2775 | SAU101576 | 5488 | SAU1c0044_orf_105p | 12554 |
| S1M10000032D11 | 2776 | SAU100128 | 5231 | #N/A | #N/A |
| S1M10000032D11 | 2776 | SAU101549 | 5476 | SAU1c0043_orf_64p | 12549 |
| S1M10000032D11 | 2776 | SAU101576 | 5488 | SAU1c0044_orf_105p | 12554 |
| S1M10000032E02 | 2777 | SAU101784 | 5530 | SAU1c0037_orf_46p | 12355 |
| S1M10000032E03 | 2778 | SAU101791 | 5532 | SAU1c0032_orf_12p | 12216 |
| S1M10000032E04 | 2779 | SAU201197 | 5806 | SAU2c0429_orf_2p | 12938 |
| S1M10000032E06 | 2780 | SAU101543 | 5473 | SAU1c0037_orf_130p | 12346 |
| S1M10000032E08 | 2781 | SAU102281 | 5633 | SAU1c0038_orf_4p | 12384 |
| S1M10000032E09 | 2782 | SAU100521 | 5283 | SAU1c0044_orf_250p | 12600 |
| S1M10000032E10 | 2783 | SAU101868 | 5565 | SAU1c0036_orf_23p | 12320 |
| S1M10000032E11 | 2784 | SAU101592 | 5490 | SAU1c0039_orf_37p | 12406 |
| S1M10000032E12 | 2785 | SAU101999 | 5585 | SAU1c0040_orf_101p | 12423 |
| S1M10000032F01 | 2786 | SAU102001 | 5586 | SAU1c0040_orf_102p | 12424 |
| S1M10000032F01 | 2786 | SAU102002 | 5587 | SAU1c0040_orf_103p | 12425 |
| S1M10000032F04 | 2787 | SAU101271 | 5411 | SAU1c0037_orf_90p | 12366 |
| S1M10000032F05 | 2788 | SAU101339 | 5422 | SAU1c0038_orf_81p | 12399 |
| S1M10000032F10 | 2789 | SAU102585 | 5703 | SAU1c0044_orf_289p | 12611 |
| S1M10000032F10 | 2789 | SAU201773 | 5834 | SAU2c0446_orf_4p | 12996 |
| S1M10000032F11 | 2790 | SAU101189 | 5392 | SAU1c0033_orf_25p | 12264 |
| S1M10000032F12 | 2791 | SAU100964 | 5363 | SAU1c0044_orf_86p | 12641 |
| S1M10000032G02 | 2792 | SAU100710 | 5311 | SAU1c0043_orf_54p | 12546 |
| S1M10000032G02 | 2792 | SAU200628 | 5788 | SAU2c0334_orf_4p | 12790 |
| S1M10000032G03 | 2793 | SAU100813 | 5334 | SAU1c0036_orf_29p | 12322 |
| S1M10000032G04 | 2794 | SAU101904 | 5573 | SAU1c0044_orf_36p | 12617 |
| S1M10000032G06 | 2795 | SAU101509 | 5469 | SAU1c0039_orf_81p | 12418 |
| S1M10000032G08 | 2796 | SAU101752 | 5522 | SAU1c0040_orf_85p | 12447 |
| S1M10000032G10 | 2797 | SAU101907 | 5574 | SAU1c0040_orf_79p | 12442 |
| S1M10000032G12 | 2798 | SAU101084 | 5377 | SAU1c0034_orf_41p | 12283 |
| S1M10000032H01 | 2799 | SAU101445 | 5452 | SAU1c0038_orf_47p | 12382 |
| S1M10000032H01 | 2799 | SAU101446 | 5453 | SAU1c0038_orf_48p | 12383 |
| S1M10000032H04 | 2800 | SAU101868 | 5565 | SAU1c0036_orf_23p | 12320 |
| S1M10000032H07 | 2801 | SAU101797 | 5537 | SAU1c0032_orf_17p | 12221 |
| S1M10000032H07 | 2801 | SAU101798 | 5538 | SAU1c0032_orf_18p | 12222 |
| S1M10000032H09 | 2802 | SAU101907 | 5574 | SAU1c0040_orf_79p | 12442 |
| S1M10000032H11 | 2803 | SAU202174 | 5845 | SAU2c0412_orf_3p | 12895 |
| S1M10000032H11 | 2803 | SAU301148 | 5888 | #N/A | #N/A |
| S1M100000323A0 | 2804 | SAU201775 | 5835 | SAU2c0446_orf_4p | 12996 |
| S1M100000323A0 | 2804 | SAU301080 | 5885 | SAU3c1287_orf_1p | 13083 |
| S1M10000033A07 | 2805 | SAU200949 | 5800 | SAU2c0380_orf_11p | 12846 |
| S1M10000033A08 | 2806 | SAU101231 | 5399 | SAU1c0035_orf_6p | 12303 |
| S1M10000033A10 | 2807 | SAU202039 | 5843 | SAU2c0452_orf_20p | 13009 |
| S1M10000033B02 | 2808 | SAU101808 | 5548 | SAU1c0032_orf_27p | 12232 |
| S1M10000033B07 | 2809 | SAU102044 | 5593 | SAU1c0039_orf_65p | 12414 |
| S1M10000033B08 | 2810 | SAU101868 | 5565 | SAU1c0036_orf_23p | 12320 |
| S1M10000033B11 | 2811 | SAU100793 | 5329 | SAU1c0028_orf_52p | 12188 |
| S1M10000033B11 | 2811 | SAU301433 | 5895 | SAU3c1420_orf_2p | 13118 |
| S1M10000033B12 | 2812 | SAU101104 | 5382 | SAU1c0029_orf_20p | 12195 |
| S1M10000033B12 | 2812 | SAU103010 | 5753 | SAU1c0029_orf_19p | 12194 |
| S1M10000033C04 | 2813 | SAU102933 | 5744 | SAU1c0039_orf_62p | 12412 |
| S1M10000033D02 | 2814 | SAU102333 | 5644 | SAU1c0045_orf_143p | 12657 |
| S1M10000033D03 | 2815 | SAU101752 | 5522 | SAU1c0040_orf_85p | 12447 |
| S1M10000033D04 | 2816 | SAU100745 | 5319 | SAU1c0044_orf_233p | 12596 |
| S1M10000033D05 | 2817 | 5AV100301 | 5254 | SAU1c0040_orf_91p | 12452 |
| S1M10000033D06 | 2818 | SAU102113 | 5601 | SAU1c0027_orf_2p | 12178 |
| S1M10000033D10 | 2819 | SAU100813 | 5334 | SAU1c0036_orf_29p | 12322 |
| S1M10000033D12 | 2820 | SAU101360 | 5431 | SAU1c0044_orf_109p | 12555 |
| S1M10000033E04 | 2821 | SAU102318 | 5643 | SAU1c0045_orf_60p | 12707 |
| S1M10000033E10 | 2822 | SAU100162 | 5239 | SAU1c0044_orf_206p | 12583 |
| S1M10000033E12 | 2823 | SAU100770 | 5324 | #N/A | #N/A |
| S1M10000033F02 | 2824 | SAU101724 | 5514 | SAU1c0016_orf_9p | 12136 |
| S1M10000033F03 | 2825 | SAU101784 | 5530 | SAU1c0037_orf_46p | 12355 |
| S1M10000033F06 | 2826 | SAU102449 | 5674 | SAU1c0045_orf_22p | 12677 |
| S1M10000033F07 | 2827 | SAU102044 | 5593 | SAU1c0039_orf_65p | 12414 |
| S1M10000033F09 | 2828 | SAU100414 | 5270 | SAU1c0022_orf_24p | 12148 |
| S1M10000033F11 | 2829 | SAU100689 | 5308 | SAU1c0036_orf_2p | 12323 |
| S1M10000033G05 | 2830 | SAU101904 | 5573 | SAU1c0044_orf_36p | 12617 |
| S1M10000033G07 | 2831 | SAU101824 | 5554 | SAU1c0038_orf_26p | 12371 |
| S1M10000033G09 | 2832 | SAU102380 | 5654 | SAU1c0033_orf_29p | 12265 |
| S1M10000033G10 | 2833 | SAU100793 | 5329 | SAU1c0028_orf_52p | 12188 |
| S1M10000033G10 | 2833 | SAU301433 | 5895 | SAU3c1420_orf_2p | 13118 |
| S1M10000033G11 | 2834 | SAU101968 | 5581 | SAU1c0028_orf_43p | 12187 |
| S1M10000033G12 | 2835 | SAU100300 | 5253 | SAU1c0040_orf_90p | 12451 |
| S1M10000033H01 | 2836 | SAU301465 | 5896 | SAU3c1429_orf_4p | 13121 |
| S1M10000033H02 | 2837 | SAU101907 | 5574 | SAU1c0040_orf_79p | 12442 |
| S1M10000033H03 | 2838 | SAU101833 | 5555 | SAU1c0038_orf_34p | 12373 |
| S1M10000033H07 | 2839 | SAU101996 | 5584 | SAU1c0040_orf_99p | 12456 |
| S1M10000033H08 | 2840 | SAU101175 | 5388 | SAU1c0031_orf_1p | 12213 |
| S1M10000033H09 | 2841 | SAU100710 | 5311 | SAU1c0043_orf_54p | 12546 |
| S1M10000033H10 | 2842 | SAU100690 | 5309 | #N/A | #N/A |
| S1M10000033H11 | 2843 | SAU102453 | 5677 | SAU1c0045_orf_19p | 12669 |
| S1M10000034A02 | 2844 | SAU101197 | 5393 | SAU1c0035_orf_60p | 12300 |
| S1M10000034A03 | 2845 | SAU102939 | 5747 | #N/A | #N/A |
| S1M10000034A04 | 2846 | SAU102578 | 5701 | SAU1c0039_orf_61p | 12411 |
| S1M10000034A05 | 2847 | SAU101242 | 5404 | SAU1c0044_orf_18p | 12578 |
| S1M10000034A08 | 2848 | SAU101020 | 5368 | SAU1c0045_orf_86p | 12710 |
| S1M10000034A09 | 2849 | SAU100773 | 5326 | SAU1c0038_orf_39p | 12377 |
| S1M10000034A11 | 2850 | SAU102389 | 5656 | SAU1c0033_orf_36p | 12268 |
| S1M10000034A12 | 2851 | SAU101632 | 5499 | SAU1c0039_orf_3p | 12407 |
| S1M10000034B03 | 2852 | SAU101907 | 5574 | SAU1c0040_orf_79p | 12442 |
| S1M10000034B05 | 2853 | SAU101630 | 5498 | SAU1c0039_orf_4p | 12410 |
| S1M10000034B06 | 2854 | SAU102607 | 5712 | SAU1c0041_orf_51p | 12472 |
| S1M10000034B06 | 2854 | SAU102944 | 5749 | SAU1c0041_orf_47p | 12468 |
| S1M10000034B07 | 2855 | SAU100077 | 5226 | SAU1c0043_orf_178p | 12520 |
| S1M10000034B08 | 2856 | SAU101341 | 5424 | SAU1c0044_orf_38p | 12618 |
| S1M10000034B09 | 2857 | SAU101909 | 5575 | SAU1c0040_orf_77p | 12441 |
| S1M10000034B10 | 2858 | SAU101882 | 5569 | SAU1c0025_orf_15p | 12163 |
| S1M10000034B12 | 2859 | SAU200593 | 5786 | SAU2c0327_orf_1p | 12784 |
| S1M10000034C02 | 2860 | SAU100557 | 5291 | SAU1c0044_orf_132p | 12565 |
| S1M10000034C06 | 2861 | SAU200157 | 5776 | #N/A | #N/A |
| S1M10000034C07 | 2862 | SAU101343 | 5425 | SAU1c0044_orf_40p | 12619 |
| S1M10000034C09 | 2863 | SAU102281 | 5633 | SAU1c0038_orf_4p | 12384 |
| S1M10000034C12 | 2864 | SAU100859 | 5342 | SAU1c0038_orf_87p | 12402 |
| S1M10000034D01 | 2865 | SAU100414 | 5270 | SAU1c0022_orf_24p | 12148 |
| S1M10000034D05 | 2866 | SAU101907 | 5574 | SAU1c0040_orf_79p | 12442 |
| S1M10000034D06 | 2867 | SAU200157 | 5776 | #N/A | #N/A |
| S1M10000034D07 | 2868 | SAU100745 | 5319 | SAU1c0044_orf_233p | 12596 |
| S1M10000034D08 | 2869 | SAU102284 | 5635 | SAU1c0038_orf_5p | 12389 |
| S1M10000034D08 | 2869 | SAU201469 | 5816 | SAU2c0438_orf_6p | 12967 |
| S1M10000034D10 | 2870 | SAU102474 | 5681 | SAU1c0026_orf_31p | 12174 |
| S1M10000034D11 | 2871 | SAU101881 | 5568 | SAU1c0025_orf_14p | 12162 |
| S1M10000034D12 | 2872 | SAU101632 | 5499 | SAU1c0039_orf_3p | 12407 |
| S1M10000034E01 | 2873 | SAU102433 | 5668 | SAU1c0045_orf_37p | 12701 |
| S1M10000034E02 | 2874 | SAU100557 | 5291 | SAU1c0044_orf_132p | 12565 |
| S1M10000034E04 | 2875 | SAU102602 | 5708 | SAU1c0032_orf_5p | 12249 |
| S1M10000034E05 | 2876 | SAU100738 | 5317 | SAU1c0044_orf_52p | 12624 |
| S1M10000034E06 | 2877 | SAU100347 | 5262 | SAU1c0036_orf_56p | 12334 |
| S1M10000034E06 | 2877 | SAU100443 | 5274 | SAU1c0036_orf_55p | 12333 |
| S1M10000034E07 | 2878 | SAU100617 | 5300 | SAU1c0035_orf_102p | 12295 |
| S1M10000034E10 | 2879 | SAU102401 | 5661 | SAU1c0030_orf_4p | 12209 |
| S1M10000034E11 | 2880 | SAU101881 | 5568 | SAU1c0025_orf_14p | 12162 |
| S1M10000034E12 | 2881 | SAU200960 | 5801 | SAU2c0377_orf_5p | 12843 |
| S1M10000034F01 | 2882 | SAU202731 | 5850 | #N/A | #N/A |
| S1M10000034F02 | 2883 | SAU201621 | 5828 | SAU2c0437_orf_4p | 12966 |
| S1M10000034F03 | 2884 | SAU201971 | 5841 | SAU2c0455_orf_17p | 13015 |
| S1M10000034F03 | 2884 | SAU301363 | 5894 | #N/A | #N/A |
| S1M10000034F04 | 2885 | SAU301620 | 5899 | SAU3c1478_orf_2p | 13140 |
| S1M10000034F05 | 2886 | SAU101630 | 5498 | SAU1c0039_orf_4p | 12410 |
| S1M10000034F07 | 2887 | SAU101175 | 5388 | SAU1c0031_orf_1p | 12213 |
| S1M10000034F08 | 2888 | SAU202736 | 5851 | SAU2c0426_orf_7p | 12927 |
| S1M10000034F09 | 2889 | SAU101869 | 5566 | SAU1c0036_orf_24p | 12321 |
| S1M10000034F10 | 2890 | SAU102350 | 5649 | SAU1c0040_orf_36p | 12433 |
| S1M10000034F12 | 2891 | SAU100522 | 5284 | SAU1c0044_orf_249p | 12599 |
| S1M10000034G02 | 2892 | SAU101543 | 5473 | SAU1c0037_orf_130p | 12346 |
| S1M10000034G03 | 2893 | SAU101198 | 5394 | SAU1c0035_orf_61p | 12301 |
| S1M10000034G06 | 2894 | SAU202174 | 5845 | SAU2c0412_orf_3p | 12895 |
| S1M10000034G07 | 2895 | SAU102380 | 5654 | SAU1c0033_orf_29p | 12265 |
| S1M10000034G08 | 2896 | SAU100158 | 5238 | SAU1c0040_orf_80p | 12443 |
| S1M10000034G09 | 2897 | SAU102294 | 5639 | SAU1c0044_orf_288p | 12610 |
| S1M10000034G09 | 2897 | SAU201775 | 5835 | SAU2c0446_orf_4p | 12996 |
| S1M10000034G11 | 2898 | SAU200558 | 5782 | SAU2c0322_orf_5p | 12777 |
| S1M10000034G12 | 2899 | SAU100557 | 5291 | SAU1c0044_orf_132p | 12565 |
| S1M10000034H01 | 2900 | SAU101293 | 5414 | SAU1c0044_orf_61p | 12631 |
| S1M10000034H02 | 2901 | SAU100414 | 5270 | SAU1c0022_orf_24p | 12148 |
| S1M10000034H03 | 2902 | SAU101571 | 5483 | SAU1c0044_orf_210p | 12585 |
| S1M10000034H06 | 2903 | SAU101570 | 5482 | SAU1c0044_orf_209p | 12584 |
| S1M10000034H07 | 2904 | SAU100077 | 5226 | SAU1c0043_orf_178p | 12520 |
| S1M10000034H08 | 2905 | SAU200740 | 5794 | SAU2c0340_orf_3p | 12798 |
| S1M10000034H09 | 2906 | SAU101791 | 5532 | SAU1c0032_orf_12p | 12216 |
| S1M10000034H10 | 2907 | SAU102422 | 5666 | SAU1c0030_orf_22p | 12207 |
| S1M10000035A03 | 2908 | SAU101360 | 5431 | SAU1c0044_orf_109p | 12555 |
| S1M10000035A08 | 2909 | SAU201403 | 5815 | SAU2c0423_orf_3p | 12913 |
| S1M10000035A09 | 2910 | SAU101350 | 5429 | SAU1c0042_orf_109p | 12487 |
| S1M10000035A09 | 2910 | SAU101351 | 5430 | SAU1c0042_orf_108p | 12486 |
| S1M10000035A10 | 2911 | SAU203296 | 5863 | SAU2c0442_orf_18p | 12983 |
| S1M10000035A11 | 2912 | SAU101756 | 5524 | SAU1c0040_orf_82p | 12445 |
| S1M10000035A12 | 2913 | SAU101455 | 5456 | SAU1c0045_orf_250p | 12686 |
| S1M10000035A12 | 2913 | SAU200916 | 5797 | SAU2c0373_orf_4p | 12838 |
| S1M10000035A12 | 2913 | SAU301620 | 5899 | SAU3c1478_orf_2p | 13140 |
| S1M10000035B01 | 2914 | SAU102584 | 5702 | SAU1c0043_orf_239p | 12537 |
| S1M10000035B03 | 2915 | SAU102246 | 5619 | SAU1c0043_orf_30p | 12542 |
| S1M10000035B04 | 2916 | SAU102246 | 5619 | SAU1c0043_orf_30p | 12542 |
| S1M10000035B08 | 2917 | SAU103232 | 5769 | SAU1c0045_orf_341p | 12697 |
| S1M10000035B11 | 2918 | SAU101756 | 5524 | SAU1c0040_orf_82p | 12445 |
| S1M10000035C01 | 2919 | SAU200928 | 5798 | SAU2c0365_orf_5p | 12815 |
| S1M10000035C02 | 2920 | SAU101039 | 5373 | SAU1c0043_orf_181p | 12522 |
| S1M10000035C04 | 2921 | SAU100214 | 5228 | SAU1c0043_orf_225p | 12535 |
| S1M10000035C06 | 2922 | SAU101497 | 5468 | SAU1c0037_orf_66p | 12361 |
| S1M10000035C11 | 2923 | SAU101752 | 5522 | SAU1c0040_orf_85p | 12447 |
| S1M10000035D01 | 2924 | SAU100414 | 5270 | SAU1c0022_orf_24p | 12148 |
| S1M10000035D04 | 2925 | SAU200928 | 5798 | SAU2c0365_orf_5p | 12815 |
| S1M10000035D06 | 2926 | SAU102117 | 5603 | SAU1c0027_orf_6p | 12181 |
| S1M10000035D09 | 2927 | SAU100970 | 5365 | SAU1c0043_orf_197p | 12529 |
| S1M10000035D12 | 2928 | SAU100608 | 5297 | SAU1c0034_orf_69p | 12293 |
| S1M10000035E02 | 2929 | SAU102883 | 5741 | SAU1c0045_orf_38p | 12702 |
| S1M10000035E03 | 2930 | SAU102447 | 5672 | SAU1c0045_orf_24p | 12685 |
| S1M10000035E04 | 2931 | SAU103025 | 5755 | SAU1c00229_orf_9p | 12202 |
| S1M10000035E08 | 2932 | SAU100690 | 5309 | #N/A | #N/A |
| S1M10000035E09 | 2933 | SAU101197 | 5393 | SAU1c0035_orf_60p | 12300 |
| S1M10000035E12 | 2934 | SAU102117 | 5603 | SAU1c0027_orf_6p | 12181 |
| S1M10000035F03 | 2935 | SAU101092 | 5381 | SAU1c0028_orf_9p | 12192 |
| S1M10000035E03 | 2935 | SAU202882 | 5855 | SAU2c0381_orf_3p | 12848 |
| S1M10000035F04 | 2936 | SAU101784 | 5530 | SAU1c0037_orf_46p | 12355 |
| S1M10000035F09 | 2937 | SAU203296 | 5863 | SAU2c0442_orf_18p | 12983 |
| S1M10000035F12 | 2938 | SAU101427 | 5447 | SAU1c0042_orf_144p | 12500 |
| S1M10000035F12 | 2938 | SAU103204 | 5767 | SAU1c0042_orf_143p | 12499 |
| S1M10000035G02 | 2939 | SAU101365 | 5432 | SAU1c0044_orf_112p | 12556 |
| S1M10000035G09 | 2940 | SAU203296 | 5863 | SAU2c0442_orf_18p | 12983 |
| S1M10000035G11 | 2941 | SAU101344 | 5426 | SAU1c0044_orf_41p | 12620 |
| S1M10000035G12 | 2942 | SAU101907 | 5574 | SAU1c0040_orf_79p | 12442 |
| S1M10000035H01 | 2943 | SAU100140 | 5235 | SAU1c0032_orf_7p | 12258 |
| S1M10000035H07 | 2944 | SAU100313 | 5259 | SAU1c0045_orf_153p | 12661 |
| S1M10000035H07 | 2944 | SAU100359 | 5264 | SAU1c0032_orf_35p | 12239 |
| S1M10000035H07 | 2944 | SAU200297 | 5778 | SAU2c0274_orf_2p | 12739 |
| S1M10000035H08 | 2945 | SAU101772 | 5526 | SAU1c0037_orf_34p | 12351 |
| S1M10000035H09 | 2946 | SAU100496 | 5279 | SAU1c0041_orf_83p | 12484 |
| S1M10000035H09 | 2946 | SAU301004 | 5882 | SAU3c1255_orf_1p | 13079 |
| S1M10000035H10 | 2947 | SAU101756 | 5524 | SAU1c0040_orf_82p | 12445 |
| S1M10000035H11 | 2948 | SAU101344 | 5426 | SAU1c0044_orf_41p | 12620 |
| S1M10000036A02 | 2949 | SAU102447 | 5672 | SAU1c0045_orf_24p | 12685 |
| S1M10000036A03 | 2950 | SAU101242 | 5404 | SAU1c0044_orf_18p | 12578 |
| S1M10000036A04 | 2951 | SAU200994 | 5802 | SAU2c0428_orf_4p | 12935 |
| S1M10000036A05 | 2952 | SAU101810 | 5549 | SAU1c0032_orf_28p | 12233 |
| S1M10000036A05 | 2952 | SAU101811 | 5550 | SAU1c0032_orf_29p | 12234 |
| S1M10000036A05 | 2952 | SAU300110 | 5865 | SAU3c0533_orf_2p | 13031 |
| S1M10000036A08 | 2953 | SAU101220 | 5396 | SAU1c0044_orf_94p | 12645 |
| S1M10000036A11 | 2954 | SAU102117 | 5603 | SAU1c0027_orf_6p | 12181 |
| S1M10000036A12 | 2955 | SAU100813 | 5334 | SAU1c0036_orf_29p | 12322 |
| S1M10000036B04 | 2956 | SAU101570 | 5482 | SAU1c0044_orf_209p | 12584 |
| S1M10000036B04 | 2956 | SAU101571 | 5483 | SAU1c0044_orf_210p | 12585 |
| S1M10000036B06 | 2957 | SAU101653 | 5504 | SAU1c0042_orf_124p | 12493 |
| S1M10000036B07 | 2958 | SAU100887 | 5350 | SAU1c0018_orf_15p | 12138 |
| S1M10000036B08 | 2959 | SAU101653 | 5504 | SAU1c0042_orf_124p | 12493 |
| S1M10000036B11 | 2960 | SAU102059 | 5597 | SAU1c0034_orf_51p | 12286 |
| S1M10000036B12 | 2961 | SAU101791 | 5532 | SAU1c0032_orf_12p | 12216 |
| S1M10000036C01 | 2962 | SAU100242 | 5246 | SAU1c0036_orf_5p | 12336 |
| S1M10000036C03 | 2963 | SAU101592 | 5490 | SAU1c0039_orf_37p | 12406 |
| S1M10000036C04 | 2964 | SAU102433 | 5668 | SAU1c0045_orf_37p | 12701 |
| S1M10000036C05 | 2965 | SAU100497 | 5280 | SAU1c0018_orf_3p | 12140 |
| S1M10000036C06 | 2966 | SAU100158 | 5238 | SAU1c0040_orf_80p | 12443 |
| S1M10000036C07 | 2967 | SAU101800 | 5540 | SAU1c0032_orf_20p | 12225 |
| S1M10000036C07 | 2967 | SAU101801 | 5541 | #N/A | #N/A |
| S1M10000036C09 | 2968 | SAU102585 | 5703 | SAU1c0044_orf_289p | 12611 |
| S1M10000036C09 | 2968 | SAU201773 | 5834 | SAU2c0446_orf_4p | 12996 |
| S1M10000036C09 | 2968 | SAU302685 | 5908 | SAU3c1403_orf_1p | 13113 |
| S1M10000036C10 | 2969 | SAU100433 | 5272 | SAU1c0040_orf_87p | 12449 |
| S1M10000036C10 | 2969 | SAU101751 | 5521 | SAU1c0040_orf_86p | 12448 |
| S1M10000036D02 | 2970 | SAU201197 | 5806 | SAU2c0429_orf_2p | 12938 |
| S1M10000036D03 | 2971 | SAU103038 | 5757 | #N/A | #N/A |
| S1M10000036D06 | 2972 | SAU103024 | 5754 | SAU1c0029_orf_6p | 12200 |
| S1M10000036D08 | 2973 | SAU101907 | 5574 | SAU1c0040_orf_79p | 12442 |
| S1M10000036D10 | 2974 | SAU102933 | 5744 | SAU1c0039_orf_62p | 12412 |
| S1M10000036D11 | 2975 | SAU101197 | 5393 | SAU1c0035_orf_60p | 12300 |
| S1M10000036D11 | 2975 | SAU101198 | 5394 | SAU1c0035_orf_61p | 12301 |
| S1M10000036D12 | 2976 | SAU102117 | 5603 | SAU1c0027_orf_6p | 12181 |
| S1M10000036E06 | 2977 | SAU100432 | 5271 | SAU1c0040_orf_88p | 12450 |
| S1M10000036E06 | 2977 | SAU202756 | 5852 | SAU2c0470_orf_1p | 13027 |
| S1M10000036E08 | 2978 | SAU101028 | 5370 | SAU1c0043_orf_7p | 12552 |
| S1M10000036E11 | 2979 | SAU101343 | 5425 | SAU1c0044_orf_40p | 12619 |
| S1M10000036F06 | 2980 | SAU101242 | 5404 | SAU1c0044_orf_18p | 12578 |
| S1M10000036F07 | 2981 | SAU200928 | 5798 | SAU2c0365_orf_5p | 12815 |
| S1M10000036F08 | 2982 | SAU200914 | 5796 | SAU2c0373_orf_2p | 12837 |
| S1M10000036F09 | 2983 | SAU100532 | 5287 | SAU1c0044_orf_198p | 12580 |
| S1M10000036F10 | 2984 | SAU101586 | 5489 | SAU1c0044_orf_242p | 12598 |
| S1M10000036F11 | 2985 | SAU201506 | 5818 | SAU2c0432_orf_18p | 12946 |
| S1M10000036G03 | 2986 | SAU101545 | 5474 | SAU1c0037_orf_132p | 12348 |
| S1M10000036G07 | 2987 | SAU102355 | 5651 | SAU1c0040_orf_40p | 12435 |
| S1M10000036G08 | 2988 | SAU102336 | 5646 | SAU1c0045_orf_146p | 12659 |
| S1M10000036G11 | 2989 | SAU101340 | 5423 | SAU1c0038_orf_82p | 12400 |
| S1M10000036H01 | 2990 | SAU101793 | 5534 | SAU1c0032_orf_14p | 12218 |
| S1M10000036H02 | 2991 | SAU102117 | 5603 | SAU1c0027_orf_6p | 12181 |
| S1M10000036H03 | 2992 | SAU102909 | 5743 | SAU1c0036_orf_16p | 12315 |
| S1M10000036H04 | 2993 | SAU102909 | 5743 | SAU1c0036_orf_16p | 12315 |
| S1M10000036H05 | 2994 | SAU101798 | 5538 | SAU1c0032_orf_18p | 12222 |
| S1M10000036H06 | 2995 | SAU102292 | 5638 | SAU1c0038_orf_10p | 12368 |
| S1M10000036H08 | 2996 | SAU102909 | 5743 | SAU1c0036_orf_16p | 12315 |
| S1M10000036H11 | 2997 | SAU101653 | 5504 | SAU1c0042_orf_124p | 12493 |
| S1M10000037A02 | 2998 | SAU101652 | 5503 | SAU1c0042_orf_123p | 12492 |
| S1M10000037A02 | 2998 | SAU101653 | 5504 | SAU1c0042_orf_124p | 12493 |
| S1M10000037A03 | 2999 | SAU100128 | 5231 | #N/A | #N/A |
| S1M10000037A03 | 2999 | SAU101549 | 5476 | SAU1c0043_orf_64p | 12549 |
| S1M10000037A03 | 2999 | SAU101576 | 5488 | SAU1c0044_orf_105p | 12554 |
| S1M10000037A06 | 3000 | SAU100964 | 5363 | SAU1c0044_orf_86p | 12641 |
| S1M10000037A08 | 3001 | SAU102669 | 5728 | SAU1c0024_orf_7p | 12160 |
| S1M10000037A09 | 3002 | SAU101455 | 5456 | SAU1c0045_orf_250p | 12686 |
| S1M10000037A09 | 3002 | SAU200916 | 5797 | SAU2c0373_orf_4p | 12838 |
| S1M10000037A11 | 3003 | SAU101436 | 5449 | SAU1c0028_orf_23p | 12183 |
| S1M10000037A12 | 3004 | SAU200914 | 5796 | SAU2c0373_orf_2p | 12837 |
| S1M10000037B03 | 3005 | SAU101999 | 5585 | SAU1c0040_orf_101p | 12423 |
| S1M10000037B04 | 3006 | SAU100767 | 5323 | SAU1c0044_orf_192p | 12579 |
| S1M10000037B05 | 3007 | SAU102578 | 5701 | SAU1c0039_orf_61p | 12411 |
| S1M10000037B06 | 3008 | SAU101806 | 5546 | SAU1c0032_orf_25p | 12230 |
| S1M10000037B06 | 3008 | SAU101807 | 5547 | SAU1c0032_orf_26p | 12231 |
| S1M10000037B07 | 3009 | SAU101915 | 5577 | SAU1c0040_orf_72p | 12439 |
| S1M10000037B08 | 3010 | SAU101592 | 5490 | SAU1c0039_orf_37p | 12406 |
| S1M10000037B10 | 3011 | SAU101346 | 5427 | SAU1c0044_orf_43p | 12621 |
| S1M10000037B11 | 3012 | SAU101399 | 5443 | SAU1c0036_orf_34p | 12325 |
| S1M10000037B12 | 3013 | SAU102117 | 5603 | SAU1c0027_orf_6p | 12181 |
| S1M10000037C05 | 3014 | SAU101482 | 5461 | SAU1c0015_orf_10p | 12123 |
| S1M10000037C06 | 3015 | SAU101653 | 5504 | SAU1c0042_orf_124p | 12493 |
| S1M10000037C07 | 3016 | SAU101641 | 5501 | SAU1c0029_orf_12p | 12193 |
| S1M10000037C08 | 3017 | SAU101752 | 5522 | SAU1c0040_orf_85p | 12447 |
| S1M10000037C09 | 3018 | SAU101818 | 5553 | SAU1c0038_orf_20p | 12369 |
| S1M10000037C10 | 3019 | SAU101752 | 5522 | SAU1c0040_orf_85p | 12447 |
| S1M10000037D04 | 3020 | SAU102283 | 5634 | SAU1c0006_orf_1p | 12119 |
| S1M10000037D05 | 3021 | SAU100114 | 5228 | SAU1c0043_orf_225p | 12535 |
| S1M10000037D06 | 3022 | SAU101996 | 5584 | SAU1c0040_orf_99p | 12456 |
| S1M10000037D09 | 3023 | SAU102246 | 5619 | SAU1c0043_orf_30p | 12542 |
| S1M10000037D12 | 3024 | SAU101999 | 5585 | SAU1c0040_orf_101p | 12423 |
| S1M10000037E02 | 3025 | SAU102447 | 5672 | SAU1c0045_orf_24p | 12685 |
| S1M10000037E02 | 3025 | SAU102448 | 5673 | SAU1c0045_orf_23p | 12681 |
| S1M10000037E03 | 3026 | SAU100813 | 5334 | SAU1c0036_orf_29p | 12322 |
| S1M10000037E06 | 3027 | SAU100921 | 5355 | SAU1c0038_orf_76p | 12396 |
| S1M10000037E08 | 3028 | SAU100139 | 5234 | SAU1c0032_orf_6p | 12255 |
| S1M10000037E08 | 3028 | SAU100140 | 5235 | SAU1c0032_orf_7p | 12258 |
| S1M10000037E09 | 3029 | SAU102049 | 5595 | SAU1c0039_orf_68p | 12416 |
| S1M10000037E10 | 3030 | SAU101444 | 5451 | SAU1c0038_orf_46p | 12381 |
| S1M10000037E11 | 3031 | SAU201571 | 5824 | SAU2c0447_orf_17p | 12997 |
| S1M10000037E12 | 3032 | SAU102602 | 5708 | SAU1c0032_orf_5p | 12249 |
| S1M10000037F02 | 3033 | SAU100776 | 5327 | SAU1c0041_orf_72p | 12482 |
| S1M10000037F03 | 3034 | SAU101339 | 5422 | SAU1c0038_orf_81p | 12399 |
| S1M10000037F04 | 3035 | SAU200468 | 5781 | SAU2c0429_orf_19p | 12937 |
| S1M10000037F05 | 3036 | SAU101807 | 5547 | SAU1c0032_orf_26p | 12231 |
| S1M10000037F06 | 3037 | SAU102585 | 5703 | SAU1c0044_orf_289p | 12611 |
| S1M10000037F06 | 3037 | SAU201773 | 5834 | SAU2c0446_orf_4p | 12996 |
| S1M10000037F07 | 3038 | SAU100793 | 5329 | SAU1c0028_orf_52p | 12188 |
| S1M10000037F07 | 3038 | SAU301433 | 5895 | SAU3c1420_orf_2p | 13118 |
| S1M10000037F08 | 3039 | SAU203001 | 5859 | SAU2c0412_orf_15p | 12894 |
| S1M10000037F08 | 3039 | SAU203007 | 5860 | SAU2c0412_orf_10p | 12893 |
| S1M10000037F09 | 3040 | SAU101592 | 5490 | SAU1c0039_orf_37p | 12406 |
| S1M10000037F10 | 3041 | SAU200468 | 5781 | SAU2c0429_orf_19p | 12937 |
| S1M10000037G01 | 3042 | SAU102502 | 5690 | SAU1c0045_orf_273p | 12689 |
| S1M10000037G01 | 3042 | SAU102503 | 5691 | SAU1c0045_orf_274p | 12690 |
| S1M10000037G02 | 3043 | SAU100658 | 5303 | SAU1c0038_orf_59p | 12388 |
| S1M10000037G03 | 3044 | SAU101344 | 5426 | SAU1c0044_orf_41p | 12620 |
| S1M10000037G06 | 3045 | SAU101752 | 5522 | SAU1c0040_orf_85p | 12447 |
| S1M10000037G07 | 3046 | SAU103038 | 5757 | #N/A | #N/A |
| S1M10000037G08 | 3047 | SAU100970 | 5365 | SAU1c0043_orf_197p | 12529 |
| S1M10000037G10 | 3048 | SAU100062 | 5225 | SAU1c0035_orf_98p | 12309 |
| S1M10000037H02 | 3049 | SAU102059 | 5597 | SAU1c0034_orf_51p | 12286 |
| S1M10000037H03 | 3050 | SAU100114 | 5228 | SAU1c0043_orf_225p | 12535 |
| S1M10000037H05 | 3051 | SAU100964 | 5363 | SAU1c0044_orf_86p | 12641 |
| S1M10000037H07 | 3052 | SAU101571 | 5483 | SAU1c0044_orf_210p | 12585 |
| S1M10000037H08 | 3053 | SAU200928 | 5798 | SAU2c0365_orf_5p | 12815 |
| S1M10000037H09 | 3054 | SAU100140 | 5235 | SAU1c0032_orf_7p | 12258 |
| S1M10000037H11 | 3055 | SAU100608 | 5297 | SAU1c0034_orf_69p | 12293 |
| S1M10000038A04 | 3056 | SAU101275 | 5412 | SAU1c0044_orf_257p | 12604 |
| S1M10000038A07 | 3057 | SAU100414 | 5270 | SAU1c0022_orf_24p | 12148 |
| S1M10000038A08 | 3058 | SAU102059 | 5597 | SAU1c0034_orf_51p | 12286 |
| S1M10000038A09 | 3059 | SAU100307 | 5257 | SAU1c0036_orf_134p | 12313 |
| S1M10000038A11 | 3060 | SAU100547 | 5290 | SAU1c0032_orf_3p | 12240 |
| S1M10000038A12 | 3061 | SAU101799 | 5539 | SAU1c0032_orf_19p | 12223 |
| S1M10000038B01 | 3062 | SAU101483 | 5462 | SAu1c0015_orf_11p | 12124 |
| S1M10000038B03 | 3063 | SAU101360 | 5431 | SAU1c0044_orf_109p | 12555 |
| S1M10000038B07 | 3064 | SAU102433 | 5668 | SAU1c0045_orf_37p | 12701 |
| S1M10000038B08 | 3065 | SAU100308 | 5258 | SAU1c0036_orf_133p | 12312 |
| S1M10000038B09 | 3066 | SAU101652 | 5503 | SAU1c0042_orf_123p | 12492 |
| S1M10000038B09 | 3066 | SAU101653 | 5504 | SAU1c0042_orf_124p | 12493 |
| S1M10000038B12 | 3067 | SAU102764 | 5734 | SAU1c0044_orf_56p | 12625 |
| S1M10000038C01 | 3068 | SAU101652 | 5503 | SAU1c0042_orf_123p | 12492 |
| S1M10000038C02 | 3069 | SAU200657 | 5789 | #N/A | #N/A |
| S1M10000038C06 | 3070 | SAU101320 | 5420 | SAU1c0015_orf_16p | 12128 |
| S1M10000038C08 | 3071 | SAU102132 | 5605 | SAU1c0027_orf_19p | 12177 |
| S1M10000038C10 | 3072 | SAU101346 | 5427 | SAU1c0044_orf_43p | 12621 |
| S1M10000038C10 | 3072 | SAU101347 | 5428 | SAU1c0044_orf_44p | 12622 |
| S1M10000038C11 | 3073 | SAU102602 | 5708 | SAU1c0032_orf_5p | 12249 |
| S1M10000038C12 | 3074 | SAU101792 | 5533 | SAU1c0032_orf_13p | 12217 |
| S1M10000038D02 | 3075 | SAU101842 | 5557 | SAU1c0042_orf_9p | 12510 |
| S1M10000038D05 | 3076 | SAU101653 | 5504 | SAU1c0042_orf_124p | 12493 |
| S1M10000038D07 | 3077 | SAU101652 | 5503 | SAU1c0042_orf_123p | 12492 |
| S1M10000038D08 | 3078 | SAU101341 | 5424 | SAU1c0044_orf_38p | 12618 |
| S1M10000038D08 | 3078 | SAU301275 | 5892 | SAU3c1365_orf_2p | 13103 |
| S1M10000038D09 | 3079 | SAU100887 | 5350 | SAU1c0018_orf_15p | 12138 |
| S1M10000038D10 | 3080 | SAU101653 | 5504 | SAU1c0042_orf_124p | 12493 |
| S1M10000038D11 | 3081 | SAU101300 | 5415 | SAU1c0044_orf_113p | 12557 |
| S1M10000038D11 | 3081 | SAU101365 | 5432 | SAU1c0044_orf_112p | 12556 |
| S1M10000038D12 | 3082 | SAU100752 | 5322 | SAU1c0043_orf_183p | 12524 |
| S1M10000038D12 | 3082 | SAU100952 | 5358 | SAU1c0043_orf_182p | 12523 |
| S1M10000038E01 | 3083 | SAU101814 | 5551 | SAU1c0032_orf_32p | 12237 |
| S1M10000038E02 | 3084 | SAU101842 | 5557 | SAU1c0042_orf_9p | 12510 |
| S1M10000038E03 | 3085 | SAU200928 | 5798 | SAU2c0365_orf_5p | 12815 |
| S1M10000038E04 | 3086 | SAU101573 | 5485 | SAU1c0044_orf_212p | 12587 |
| S1M10000038E05 | 3087 | SAU101653 | 5504 | SAU1c0042_orf_124p | 12493 |
| S1M10000038E06 | 3088 | SAU102231 | 5614 | SAU1c0043_orf_18p | 12527 |
| S1M10000038E06 | 3088 | SAU102232 | 5615 | SAU1c0043_orf_19p | 12530 |
| S1M10000038E07 | 3089 | SAU200593 | 5786 | SAU2c0327_orf_1p | 12784 |
| S1M10000038E10 | 3090 | SAU201558 | 5823 | SAU2c0434_orf_5p | 12954 |
| S1M10000038E12 | 3091 | SAU100838 | 5337 | SAU1c0031_orf_12p | 12211 |
| S1M10000038E12 | 3091 | SAU100839 | 5338 | SAU1c0031_orf_11p | 12210 |
| S1M10000038F03 | 3092 | SAU102117 | 5603 | SAU1c0027_orf_6p | 12181 |
| S1M10000038F04 | 3093 | SAU100964 | 5363 | SAU1c0044_orf_86p | 12641 |
| S1M10000038F04 | 3093 | SAU100965 | 5364 | SAU1c0044_orf_87p | 12642 |
| S1M10000038F05 | 3094 | SAU100964 | 5363 | SAU1c0044_orf_86p | 12641 |
| S1M10000038F05 | 3094 | SAU100965 | 5364 | SAU1c0044_orf_87p | 12642 |
| S1M10000038F06 | 3095 | SAU101189 | 5392 | SAU1c0033_orf_25p | 12264 |
| S1M10000038F08 | 3096 | SAU101752 | 5522 | SAU1c0040_orf_85p | 12447 |
| S1M10000038F09 | 3097 | SAU201666 | 5830 | SAU2c0442_orf_11p | 12981 |
| S1M10000038F10 | 3098 | SAU101197 | 5393 | SAU1c0035_orf_60p | 12300 |
| S1M10000038F11 | 3099 | SAU100747 | 5320 | SAU1c0044_orf_235p | 12597 |
| S1M10000038F12 | 3100 | SAU202039 | 5843 | SAU2c0452_orf_20p | 13009 |
| S1M10000038G01 | 3101 | SAU101271 | 5411 | SAU1c0037_orf_90p | 12366 |
| S1M10000038G03 | 3102 | SAU100158 | 5238 | SAU1c0040_orf_80p | 12443 |
| S1M10000038G04 | 3103 | SAU100475 | 5276 | SAU1c0036_orf_61p | 12337 |
| S1M10000038G06 | 3104 | SAU101189 | 5392 | SAU1c0033_orf_25p | 12264 |
| S1M10000038G08 | 3105 | SAU200928 | 5798 | SAU2c0365_orf_5p | 12815 |
| S1M10000038G10 | 3106 | SAU102602 | 5708 | SAU1c0032_orf_5p | 12249 |
| S1M10000038G11 | 3107 | SAU100123 | 5230 | SAU1c0043_orf_189p | 12526 |
| S1M10000038G11 | 3107 | SAU102001 | 5586 | SAU1c0040_orf_102p | 12424 |
| S1M10000038G12 | 3108 | SAU101184 | 5391 | SAU1c0035_orf_80p | 12305 |
| S1M10000038H03 | 3109 | SAU101798 | 5538 | SAU1c0032_orf_18p | 12222 |
| S1M10000038H07 | 3110 | SAU101752 | 5522 | SAU1c0040_orf_85p | 12447 |
| S1M10000038H09 | 3111 | SAU102340 | 5647 | SAU1c0045_orf_149p | 12660 |
| S1M10000038H11 | 3112 | SAU101452 | 5455 | SAU1c0045_orf_247p | 12684 |
| S1M10000039A02 | 3113 | SAU100496 | 5279 | SAU1c0041_orf_83p | 12484 |
| S1M10000039A02 | 3113 | SAU301004 | 5882 | SAU3c1255_orf_1p | 13079 |
| S1M10000039A05 | 3114 | SAU100964 | 5363 | SAU1c0044_orf_86p | 12641 |
| S1M10000039A05 | 3114 | SAU100965 | 5364 | SAU1c0044_orf_87p | 12642 |
| S1M10000039A07 | 3115 | SAU100131 | 5232 | SAU1c0043_orf_156p | 12517 |
| S1M10000039A08 | 3116 | SAU100522 | 5284 | SAU1c0044_orf_249p | 12599 |
| S1M10000039A11 | 3117 | SAU100613 | 5299 | SAU1c0015_orf_14p | 12126 |
| S1M10000039A12 | 3118 | SAU301465 | 5896 | SAU3c1429_orf_4p | 13121 |
| S1M10000039B02 | 3119 | SAU101455 | 5456 | SAU1c0045_orf_250p | 12686 |
| S1M10000039B02 | 3119 | SAU200916 | 5797 | SAU2c0373_orf_4p | 12838 |
| S1M10000039B06 | 3120 | SAU102350 | 5649 | SAU1c0040_orf_36p | 12433 |
| S1M10000039B07 | 3121 | SAU101869 | 5566 | SAU1c0036_orf_24p | 12321 |
| S1M10000039B10 | 3122 | SAU101752 | 5522 | SAU1c0040_orf_85p | 12447 |
| S1M10000039B12 | 3123 | SAU301118 | 5886 | SAU3c1305_orf_3p | 13086 |
| S1M10000039C04 | 3124 | SAU102252 | 5621 | SAU1c0032_orf_48p | 12241 |
| S1M10000039C06 | 3125 | SAU100633 | 5301 | SAU1c0043_orf_147p | 12515 |
| S1M10000039C07 | 3126 | SAU200657 | 5789 | #N/A | #N/A |
| S1M10000039C08 | 3127 | SAU200468 | 5781 | SAU2c0429_orf_19p | 12937 |
| S1M10000039C09 | 3128 | SAU100414 | 5270 | SAU1c0022_orf_24p | 12148 |
| S1M10000039C10 | 3129 | SAU101543 | 5473 | SAU1c0037_orf_130p | 12346 |
| S1M10000039C11 | 3130 | SAU200657 | 5789 | #N/A | #N/A |
| S1M10000039D02 | 3131 | SAU201403 | 5815 | SAU2c0423_orf_3p | 12913 |
| S1M10000039D09 | 3132 | SAU102294 | 5639 | SAU1c0044_orf_288p | 12610 |
| S1M10000039D09 | 3132 | SAU301080 | 5885 | SAU3c1287_orf_1p | 13083 |
| S1M10000039D10 | 3133 | SAU100323 | 5261 | SAU1c0044_orf_171p | 12575 |
| S1M10000039E01 | 3134 | SAU102264 | 5628 | SAU1c0032_orf_60p | 12250 |
| S1M10000039E08 | 3135 | SAU100412 | 5269 | SAU1c0029_orf_38p | 12197 |
| S1M10000039E09 | 3136 | SAU100056 | 5223 | SAU1c0044_orf_176p | 12577 |
| S1M10000039E10 | 3137 | SAU102394 | 5659 | SAU1c0033_orf_41p | 12271 |
| S1M10000039E10 | 3137 | SAU301118 | 5886 | SAU3c1305_orf_3p | 13086 |
| S1M10000039E11 | 3138 | SAU102473 | 5680 | SAU1c0026_orf_30p | 12173 |
| S1M10000039F02 | 3139 | SAU201571 | 5824 | SAU2c0447_orf_17p | 12997 |
| S1M10000039F03 | 3140 | SAU102527 | 5693 | SAU1c0032_orf_9p | 12260 |
| S1M10000039F05 | 3141 | SAU100118 | 5229 | SAU1c0015_orf_13p | 12125 |
| S1M10000039F07 | 3142 | SAU102531 | 5694 | SAU1c0045_orf_186p | 12667 |
| S1M10000039F08 | 3143 | SAU100158 | 5238 | SAU1c0040_orf_80p | 12443 |
| S1M10000039F09 | 3144 | SAU200157 | 5776 | #N/A | #N/A |
| S1M10000039F10 | 3145 | SAU100059 | 5224 | SAU1c0045_orf_10p | 12652 |
| S1M10000039F12 | 3146 | SAU101565 | 5480 | SAU1c0022_orf_8p | 12151 |
| S1M10000039G03 | 3147 | SAU101653 | 5504 | SAU1c0042_orf_124p | 12493 |
| S1M10000039G04 | 3148 | SAU102292 | 5638 | SAU1c0038_orf_10p | 12368 |
| S1M10000039G07 | 3149 | SAU100952 | 5358 | SAU1c0043_orf_182p | 12523 |
| S1M10000039G07 | 3149 | SAU101039 | 5373 | SAU1c0043_orf_181p | 12522 |
| S1M10000039G10 | 3150 | SAU101815 | 5552 | SAU1c0032_orf_33p | 12238 |
| S1M10000039H02 | 3151 | SAU102585 | 5703 | SAU1c0044_orf_289p | 12611 |
| S1M10000039H02 | 3151 | SAU201773 | 5834 | SAU2c0446_orf_4p | 12996 |
| S1M10000039H03 | 3152 | SAU100313 | 5259 | SAU1c0045_orf_153p | 12661 |
| S1M10000039H03 | 3152 | SAU100359 | 5264 | SAU1c0032_orf_35p | 12239 |
| S1M10000039H03 | 3152 | SAU200297 | 5778 | SAU2c0274_orf_2p | 12739 |
| S1M10000039H04 | 3153 | SAU101752 | 5522 | SAU1c0040_orf_85p | 12447 |
| S1M10000039H06 | 3154 | SAU102283 | 5634 | SAU1c0006_orf_1p | 12119 |
| S1M10000039H07 | 3155 | SAU100793 | 5329 | SAU1c0028_orf_52p | 12188 |
| S1M10000039H07 | 3155 | SAU301433 | 5895 | SAU3c1420_orf_2p | 13118 |
| S1M10000039H08 | 3156 | SAU102440 | 5671 | SAU1c0045_orf_30p | 12692 |
| S1M10000040A04 | 3157 | SAU100040 | 5221 | SAU1c0043_orf_217p | 12533 |
| S1M10000040A05 | 3158 | SAU102671 | 5729 | SAU1c0024_orf_9p | 12161 |
| S1M10000040A07 | 3159 | SAU101028 | 5370 | SAU1c0043_orf_7p | 12552 |
| S1M10000040A08 | 3160 | SAU200157 | 5776 | #N/A | #N/A |
| S1M10000040A10 | 3161 | SAU103038 | 5757 | #N/A | #N/A |
| S1M10000040A11 | 3162 | SAU101801 | 5541 | #N/A | #N/A |
| S1M10000040B01 | 3163 | SAU101461 | 5457 | SAU1c0045_orf_234p | 12680 |
| S1M10000040B03 | 3164 | SAU102102 | 5600 | SAU1c0045_orf_340p | 12696 |
| S1M10000040B07 | 3165 | SAU101432 | 5448 | SAU1c0028_orf_27p | 12184 |
| S1M10000040B11 | 3166 | SAU101198 | 5394 | SAU1c0035_orf_61p | 12301 |
| S1M10000040C03 | 3167 | SAU201971 | 5841 | SAU2c0455_orf_17p | 13015 |
| S1M10000040C03 | 3167 | SAU301363 | 5894 | #N/A | #N/A |
| S1M10000040C04 | 3168 | SAU102551 | 5698 | SAU1c0045_orf_206p | 12672 |
| S1M10000040C05 | 3169 | SAU102534 | 5696 | #N/A | #N/A |
| S1M10000040C06 | 3170 | SAU101247 | 5405 | SAU1c0043_orf_136p | 12512 |
| S1M10000040C07 | 3171 | SAU100970 | 5365 | SAU1c0043_orf_197p | 12529 |
| S1M10000040C08 | 3172 | SAU101197 | 5393 | SAU1c0035_orf_60p | 12300 |
| S1M10000040C10 | 3173 | SAU201810 | 5836 | SAU2c0308_orf_2p | 12769 |
| S1M10000040C10 | 3173 | SAU202174 | 5845 | SAU2c0412_orf_3p | 12895 |
| S1M10000040C10 | 3173 | SAU301148 | 5888 | #N/A | #N/A |
| S1M10000040C11 | 3174 | SAU101869 | 5566 | SAU1c0036_orf_24p | 12321 |
| S1M10000040D01 | 3175 | SAU101806 | 5546 | SAU1c0032_orf_25p | 12230 |
| S1M10000040D01 | 3175 | SAU101807 | 5547 | SAU1c0032_orf_26p | 12231 |
| S1M10000040D03 | 3176 | SAU102200 | 5611 | SAU1c0045_orf_168p | 12665 |
| S1M10000040D03 | 3176 | SAU102201 | 5612 | SAU1c0045_orf_169p | 12666 |
| S1M10000040D08 | 3177 | SAU100633 | 5301 | SAU1c0043_orf_147p | 12515 |
| S1M10000040D09 | 3178 | SAU101632 | 5499 | SAU1c0039_orf_3p | 12407 |
| S1M10000040D11 | 3179 | SAU101546 | 5475 | SAU1c0037_orf_133p | 12349 |
| S1M10000040E01 | 3180 | SAU100916 | 5353 | SAU1c0038_orf_71p | 12394 |
| S1M10000040E02 | 3181 | SAU101845 | 5558 | SAU1c0042_orf_7p | 12506 |
| S1M10000040E04 | 3182 | SAU101546 | 5475 | SAU1c0037_orf_133p | 12349 |
| S1M10000040E05 | 3183 | SAU101632 | 5499 | SAU1c0039_orf_3p | 12407 |
| S1M10000040E06 | 3184 | SAU101545 | 5474 | SAU1c0037_orf_132p | 12348 |
| S1M10000040E07 | 3185 | SAU101006 | 5367 | SAU1c0028_orf_59p | 12190 |
| S1M10000040E09 | 3186 | SAU102605 | 5710 | SAU1c0041_orf_49p | 12470 |
| S1M10000040E10 | 3187 | SAU100714 | 5312 | SAU1c0044_orf_74p | 12635 |
| S1M10000040E11 | 3188 | SAU101226 | 5398 | SAU1c0035_orf_2p | 12298 |
| S1M10000040E12 | 3189 | SAU102503 | 5691 | SAU1c0045_orf_274p | 12690 |
| S1M10000040E12 | 3189 | SAU201380 | 5812 | SAU2c0426_orf_11p | 12922 |
| S1M10000040F01 | 3190 | SAU101226 | 5398 | SAU1c0035_orf_2p | 12298 |
| S1M10000040F02 | 3191 | SAU101614 | 5494 | SAU1c0044_orf_9p | 12649 |
| S1M10000040F03 | 3192 | SAU101592 | 5490 | SAU1c0039_orf_37p | 12406 |
| S1M10000040F04 | 3193 | SAU100123 | 5230 | SAU1c0043_orf_189p | 12526 |
| S1M10000040F04 | 3193 | SAU102001 | 5586 | SAU1c0040_orf_102p | 12424 |
| S1M10000040F04 | 3193 | SAU103159 | 5762 | SAU1c0045_orf_204p | 12670 |
| S1M10000040F04 | 3193 | SAU201827 | 5837 | SAU2c0449_orf_21p | 13002 |
| S1M10000040F05 | 3194 | SAU102232 | 5615 | SAU1c0043_orf_19p | 12530 |
| S1M10000040F06 | 3195 | SAU100547 | 5290 | SAU1c0032_orf_3p | 12240 |
| S1M10000040F08 | 3196 | SAU300713 | 5875 | SAU3c1104_orf_1p | 13058 |
| S1M10000040F09 | 3197 | SAU101610 | 5492 | SAU1c0044_orf_5p | 12629 |
| S1M10000040F12 | 3198 | SAU101752 | 5522 | SAU1c0040_orf_85p | 12447 |
| S1M10000040G01 | 3199 | SAU200006 | 5770 | SAU2c0157_orf_1p | 12723 |
| S1M10000040G02 | 3200 | SAU200561 | 5783 | SAU2c0324_orf_3p | 12779 |
| S1M10000040G02 | 3200 | SAU301773 | 5901 | SAU3c1509_orf_2p | 13157 |
| S1M10000040G04 | 3201 | SAU100414 | 5270 | SAU1c0022_orf_24p | 12148 |
| S1M10000040G07 | 3202 | SAU101543 | 5473 | SAU1c0037_orf_130p | 12346 |
| S1M10000040G08 | 3203 | SAU101752 | 5522 | SAU1c0040_orf_85p | 12447 |
| S1M10000040G12 | 3204 | SAU101421 | 5446 | SAU1c0042_orf_138p | 12498 |
| S1M10000040H02 | 3205 | SAU100773 | 5326 | SAU1c0038_orf_39p | 12377 |
| S1M10000040H03 | 3206 | SAU100414 | 5270 | SAU1c0022_orf_24p | 12148 |
| S1M10000040H04 | 3207 | SAU200914 | 5796 | SAU2c0373_orf_2p | 12837 |
| S1M10000040H05 | 3208 | SAU101400 | 5444 | SAU1c0036_orf_35p | 12326 |
| S1M10000040H07 | 3209 | SAU100921 | 5355 | SAU1c0038_orf_76p | 12396 |
| S1M10000040H10 | 3210 | SAU202039 | 5843 | SAU2c0452_orf_20p | 13009 |
| S1M10000041A03 | 3211 | SAU102054 | 5596 | SAU1c0039_orf_74p | 12417 |
| S1M10000041B02 | 3212 | SAU101592 | 5490 | SAU1c0039_orf_37p | 12406 |
| S1M10000041B03 | 3213 | SAU101592 | 5490 | SAU1c0039_orf_37p | 12406 |
| S1M10000041B05 | 3214 | SAU101798 | 5538 | SAU1c0032_orf_18p | 12222 |
| S1M10000041B06 | 3215 | SAU301620 | 5899 | SAU3c1478_orf_2p | 13140 |
| S1M10000041B07 | 3216 | SAU101145 | 5384 | SAU1c0035_orf_43p | 12299 |
| S1M10000041B12 | 3217 | SAU102725 | 5733 | SAU1c0036_orf_68p | 12338 |
| S1M10000041C08 | 3218 | SAU102607 | 5712 | SAU1c0041_orf_51p | 12472 |
| S1M10000041C08 | 3218 | SAU102944 | 5749 | SAU1c0041_orf_47p | 12468 |
| S1M10000041C10 | 3219 | SAU101784 | 5530 | SAU1c0037_orf_46p | 12355 |
| S1M10000041C11 | 3220 | SAU101570 | 5482 | SAU1c0044_orf_209p | 12584 |
| S1M10000041D06 | 3221 | SAU101777 | 5527 | SAU1c0037_orf_39p | 12352 |
| S1M10000041D07 | 3222 | SAU102639 | 5724 | #N/A | #N/A |
| S1M10000041D08 | 3223 | SAU200030 | 5772 | SAU2c0282_orf_3p | 12745 |
| S1M10000041D10 | 3224 | SAU101573 | 5485 | SAU1c0044_orf_212p | 12587 |
| S1M10000041D12 | 3225 | SAU102658 | 5726 | SAU1c0045_orf_121p | 12654 |
| S1M10000041E03 | 3226 | SAU101573 | 5485 | SAU1c0044_orf_212p | 12587 |
| S1M10000041E06 | 3227 | SAU101996 | 5584 | SAU1c0040_orf_99p | 12456 |
| S1M10000041E09 | 3228 | SAU201236 | 5808 | SAU2c0409_orf_10p | 12891 |
| S1M10000041E12 | 3229 | SAU100952 | 5358 | SAU1c0043_orf_182p | 12523 |
| S1M10000041F03 | 3230 | SAU101571 | 5483 | SAU1c0044_orf_210p | 12585 |
| S1M10000041F03 | 3230 | SAU101572 | 5484 | SAU1c0044_orf_211p | 12586 |
| S1M10000041F11 | 3231 | SAU102117 | 5603 | SAU1c0027_orf_6p | 12181 |
| S1M10000041E12 | 3232 | SAU102480 | 5684 | SAU1c0039_orf_100p | 12404 |
| S1M10000041F12 | 3232 | SAU102481 | 5685 | SAU1c0039_orf_99p | 12422 |
| S1M10000041G01 | 3233 | SAU100532 | 5287 | SAU1c0044_orf_198p | 12580 |
| S1M10000041G06 | 3234 | SAU102345 | 5648 | SAU1c0045_orf_125p | 12655 |
| S1M10000041G08 | 3235 | SAU101546 | 5475 | SAU1c0037_orf_133p | 12349 |
| S1M10000041G10 | 3236 | SAU100866 | 5344 | SAU1c0044_orf_100p | 12553 |
| S1M10000041G11 | 3237 | SAU101802 | 5542 | SAU1c0032_orf_22p | 12227 |
| S1M10000041H01 | 3238 | SAU101198 | 5394 | SAU1c0035_orf_61p | 12301 |
| S1M10000041H04 | 3239 | SAU100497 | 5280 | SAU1c0018_orf_3p | 12140 |
| S1M10000041H05 | 3240 | SAU100242 | 5246 | SAU1c0036_orf_5p | 12336 |
| S1M10000041H07 | 3241 | SAU102486 | 5687 | SAU1c0039_orf_93p | 12420 |
| S1M10000041H07 | 3241 | SAU102487 | 5688 | SAU1c0039_orf_92p | 12419 |
| S1M10000041H08 | 3242 | SAU301133 | 5887 | SAU3c1311_orf_3p | 13087 |
| S1M10000041H09 | 3243 | SAU103169 | 5763 | SAU1c0045_orf_230p | 12678 |
| S1M10000042A04 | 3244 | SAU201236 | 5808 | SAU2c0409_orf_10p | 12891 |
| S1M10000042A05 | 3245 | SAU102433 | 5668 | SAU1c0045_orf_37p | 12701 |
| S1M10000042A06 | 3246 | SAU102578 | 5701 | SAU1c0039_orf_61p | 12411 |
| S1M10000042A07 | 3247 | SAU100633 | 5301 | SAU1c0043_orf_147p | 12515 |
| S1M10000042A09 | 3248 | SAU101495 | 5467 | SAU1c0037_orf_65p | 12360 |
| S1M10000042A11 | 3249 | SAU101815 | 5552 | SAU1c0032_orf_33p | 12238 |
| S1M10000042A12 | 3250 | SAU101632 | 5499 | SAU1c0039_orf_3p | 12407 |
| S1M10000042B02 | 3251 | SAU202736 | 5851 | SAU2c0426_orf_7p | 12927 |
| S1M10000042B03 | 3252 | SAU101907 | 5574 | SAU1c0040_orf_79p | 12442 |
| S1M10000042B06 | 3253 | SAU101652 | 5503 | SAU1c0042_orf_123p | 12492 |
| S1M10000042B07 | 3254 | SAU101343 | 5425 | SAU1c0044_orf_40p | 12619 |
| S1M10000042B08 | 3255 | SAU100443 | 5274 | SAU1c0036_orf_55p | 12333 |
| S1M10000042B09 | 3256 | SAU101802 | 5542 | SAU1c0032_orf_22p | 12227 |
| S1M10000042B10 | 3257 | SAU100141 | 5236 | SAU1c0032_orf_8p | 12259 |
| S1M10000042B10 | 3257 | SAU102527 | 5693 | SAU1c0032_orf_9p | 12260 |
| S1M10000042B11 | 3258 | SAU101815 | 5552 | SAU1c0032_orf_33p | 12238 |
| S1M10000042B12 | 3259 | SAU101653 | 5504 | SAU1c0042_orf_124p | 12493 |
| S1M10000042C02 | 3260 | SAU100617 | 5300 | SAU1c0035_orf_102p | 12295 |
| S1M10000042C06 | 3261 | SAU102032 | 5591 | SAU1c0029_orf_47p | 12198 |
| S1M10000042C10 | 3262 | SAU101495 | 5467 | SAU1c0037_orf_65p | 12360 |
| S1M10000042C11 | 3263 | SAU103037 | 5756 | SAU1c0044_orf_303p | 12613 |
| S1M10000042D04 | 3264 | SAU101571 | 5483 | SAU1c0044_orf_210p | 12585 |
| S1M10000042D07 | 3265 | SAU101632 | 5499 | SAU1c0039_orf_3p | 12407 |
| S1M10000042D10 | 3266 | SAU203296 | 5863 | SAU2c0442_orf_18p | 12983 |
| S1M10000042D11 | 3267 | SAU102663 | 5727 | SAU1c0024_orf_2p | 12158 |
| S1M10000042E03 | 3268 | SAU101495 | 5467 | SAU1c0037_orf_65p | 12360 |
| S1M10000042E06 | 3269 | SAU102433 | 5668 | SAU1c0045_orf_37p | 12701 |
| S1M10000042E08 | 3270 | SAU103198 | 5766 | #N/A | #N/A |
| S1M10000042F01 | 3271 | SAU102117 | 5603 | SAU1c0027_orf_6p | 12181 |
| S1M10000042F02 | 3272 | SAU101891 | 5571 | SAU1c0034_orf_30p | 12281 |
| S1M10000042F05 | 3273 | SAU101652 | 5503 | SAU1c0042_orf_123p | 12492 |
| S1M10000042F06 | 3274 | SAU100773 | 5326 | SAU1c0038_orf_39p | 12377 |
| S1M10000042F08 | 3275 | SAU100162 | 5239 | SAU1c0044_orf_206p | 12583 |
| S1M10000042F09 | 3276 | SAU100246 | 5247 | SAU1c0042_orf_130p | 12496 |
| S1M10000042F09 | 3276 | SAU300998 | 5881 | SAU3c1253_orf_3p | 13077 |
| S1M10000042F10 | 3277 | SAU102602 | 5708 | SAU1c0032_orf_5p | 12249 |
| S1M10000042F11 | 3278 | SAU101653 | 5504 | SAU1c0042_orf_124p | 12493 |
| S1M10000042G01 | 3279 | SAU100140 | 5235 | SAU1c0032_orf_7p | 12258 |
| S1M10000042G03 | 3280 | SAU101220 | 5396 | SAU1c0044_orf_94p | 12645 |
| S1M10000042G08 | 3281 | SAU101907 | 5574 | SAU1c0040_orf_79p | 12442 |
| S1M10000042G09 | 3282 | SAU100158 | 5238 | SAU1c0040_orf_80p | 12443 |
| S1M10000042G12 | 3283 | SAU100521 | 5283 | SAU1c0044_orf_250p | 12600 |
| S1M10000042H05 | 3284 | SAU101491 | 5464 | SAU1c0025_orf_20p | 12165 |
| S1M10000042H07 | 3285 | SAU100433 | 5272 | SAU1c0040_orf_87p | 12449 |
| S1M10000042H11 | 3286 | SAU101632 | 5499 | SAU1c0039_orf_3p | 12407 |
| S1M10000043A02 | 3287 | SAU203001 | 5859 | SAU2c0412_orf_15p | 12894 |
| S1M10000043A03 | 3288 | SAU101400 | 5444 | SAU1c0036_orf_35p | 12326 |
| S1M10000043A04 | 3289 | SAU200088 | 5775 | SAU2c0159_orf_1p | 12724 |
| S1M10000043A06 | 3290 | SAU100077 | 5226 | SAU1c0043_orf_178p | 12520 |
| S1M10000043A07 | 3291 | SAU101752 | 5522 | SAU1c0040_orf_85p | 12447 |
| S1M10000043A08 | 3292 | SAU101543 | 5473 | SAU1c0037_orf_130p | 12346 |
| S1M10000043A10 | 3293 | SAU100865 | 5343 | SAU1c0044_orf_99p | 12648 |
| S1M10000043A11 | 3294 | SAU100865 | 5343 | SAU1c0044_orf_99p | 12648 |
| S1M10000043A12 | 3295 | SAU100887 | 5350 | SAU1c0018_orf_15p | 12138 |
| S1M10000043B01 | 3296 | SAU102059 | 5597 | SAU1c0034_orf_51p | 12286 |
| S1M10000043B02 | 3297 | SAU100059 | 5224 | SAU1c0045_orf_10p | 12652 |
| S1M10000043B07 | 3298 | SAU101922 | 5578 | SAU1c0040_orf_66p | 12438 |
| S1M10000043B07 | 3298 | SAU200345 | 5779 | SAU2c0292_orf_3p | 12751 |
| S1M10000043B08 | 3299 | SAU100313 | 5259 | SAU1c0045_orf_153p | 12661 |
| S1M10000043B08 | 3299 | SAU100359 | 5264 | SAU1c0032_orf_35p | 12239 |
| S1M10000043B08 | 3299 | SAU200297 | 5778 | SAU2c0274_orf_2p | 12739 |
| S1M10000043B09 | 3300 | SAU100521 | 5283 | SAU1c0044_orf_250p | 12600 |
| S1M10000043B10 | 3301 | SAU100436 | 5273 | SAU1c0023_orf_20p | 12154 |
| S1M10000043B12 | 3302 | SAU102142 | 5606 | SAU1c0041_orf_13p | 12457 |
| S1M10000043C02 | 3303 | SAU101777 | 5527 | SAU1c0037_orf_39p | 12352 |
| S1M10000043C07 | 3304 | SAU101784 | 5530 | SAU1c0037_orf_46p | 12355 |
| S1M10000043C11 | 3305 | SAU201403 | 5815 | SAU2c0423_orf_3p | 12913 |
| S1M10000043C12 | 3306 | SAU102059 | 5597 | SAU1c0034_orf_51p | 12286 |
| S1M10000043D01 | 3307 | SAU100866 | 5344 | SAU1c0044_orf_100p | 12553 |
| S1M10000043D02 | 3308 | SAU301465 | 5896 | SAU3c1429_orf_4p | 13121 |
| S1M10000043D04 | 3309 | SAU200928 | 5798 | SAU2c0365_orf_5p | 12815 |
| S1M10000043D10 | 3310 | SAU102631 | 5721 | SAU1c0045_orf_94p | 12712 |
| S1M10000043D12 | 3311 | SAU100496 | 5279 | SAU1c0041_orf_83p | 12484 |
| S1M10000043D12 | 3311 | SAU301004 | 5882 | SAU3c1255_orf_1p | 13079 |
| S1M10000043E02 | 3312 | SAU100793 | 5329 | SAU1c0028_orf_52p | 12188 |
| S1M10000043E02 | 3312 | SAU301433 | 5895 | SAU3c1420_orf_2p | 13118 |
| S1M10000043E03 | 3313 | SAU102032 | 5591 | SAU1c0029_orf_47p | 12198 |
| S1M10000043E05 | 3314 | SAU102067 | 5598 | SAU1c0034_orf_54p | 12287 |
| S1M10000043E07 | 3315 | SAU102117 | 5603 | SAU1c0027_orf_6p | 12181 |
| S1M10000043E08 | 3316 | SAU101344 | 5426 | SAU1c0044_orf_41p | 12620 |
| S1M10000043E10 | 3317 | SAU100186 | 5242 | SAU1c0036_orf_19p | 12317 |
| S1M10000043E11 | 3318 | SAU102498 | 5689 | SAU1c0045_orf_270p | 12688 |
| S1M10000043E11 | 3318 | SAU201381 | 5813 | SAU2c0426_orf_16p | 12923 |
| S1M10000043E12 | 3319 | SAU101752 | 5522 | SAU1c0040_orf_85p | 12447 |
| S1M10000043F01 | 3320 | SAU101797 | 5537 | SAU1c0032_orf_17p | 12221 |
| S1M10000043F01 | 3320 | SAU101798 | 5538 | SAU1c0032_orf_18p | 12222 |
| S1M10000043F05 | 3321 | SAU101543 | 5473 | SAU1c0037_orf_130p | 12346 |
| S1M10000043F07 | 3322 | SAU102447 | 5672 | SAU1c0045_orf_24p | 12685 |
| S1M10000043F07 | 3322 | SAU102448 | 5673 | SAU1c0045_orf_23p | 12681 |
| S1M10000043F08 | 3323 | SAU101344 | 5426 | SAU1c0044_orf_41p | 12620 |
| S1M10000043F09 | 3324 | SAU101801 | 5541 | #N/A | #N/A |
| S1M10000043G01 | 3325 | SAU100059 | 5224 | SAU1c0045_orf_10p | 12652 |
| S1M10000043G04 | 3326 | SAU102423 | 5667 | SAU1c0030_orf_23p | 12208 |
| S1M10000043G05 | 3327 | SAU102602 | 5708 | SAU1c0032_orf_5p | 12249 |
| S1M10000043G09 | 3328 | SAU102585 | 5703 | SAU1c0044_orf_289p | 12611 |
| S1M10000043G09 | 3328 | SAU201773 | 5834 | SAU2c0446_orf_4p | 12996 |
| S1M10000043G10 | 3329 | SAU100158 | 5238 | SAU1c0040_orf_80p | 12443 |
| S1M10000043H01 | 3330 | SAU101797 | 5537 | SAU1c0032_orf_17p | 12221 |
| S1M10000043H01 | 3330 | SAU101798 | 5538 | SAU1c0032_orf_18p | 12222 |
| S1M10000043H03 | 3331 | SAU101803 | 5543 | SAU1c0032_orf_23p | 12228 |
| S1M10000043H03 | 3331 | SAU101804 | 5544 | #N/A | #N/A |
| S1M10000043H04 | 3332 | SAU100128 | 5231 | #N/A | #N/A |
| S1M10000043H04 | 3332 | SAU101549 | 5476 | SAU1c0043_orf_64p | 12549 |
| S1M10000043H04 | 3332 | SAU101576 | 5488 | SAU1c0044_orf_105p | 12554 |
| S1M10000043H05 | 3333 | SAU200058 | 5773 | SAU2c0134_orf_1p | 12719 |
| S1M10000043H05 | 3333 | SAU200059 | 5774 | SAU2c0134_orf_3p | 12720 |
| S1M10000043H06 | 3334 | SAU102417 | 5663 | SAU1c0030_orf_17p | 12204 |
| S1M10000043H06 | 3334 | SAU102863 | 5737 | #N/A | #N/A |
| S1M10000043H09 | 3335 | SAU302950 | 5914 | SAU3c1512_orf_12p | 13160 |
| S1M10000043H10 | 3336 | SAU101024 | 5369 | SAU1c0045_orf_90p | 12711 |
| S1M10000043H11 | 3337 | SAU101907 | 5574 | SAU1c0040_orf_79p | 12442 |
| S1M10000044A02 | 3338 | SAU101092 | 5381 | SAU1c0028_orf_9p | 12192 |
| S1M10000044A06 | 3339 | SAU101777 | 5527 | SAU1c0037_orf_39p | 12352 |
| S1M10000044A08 | 3340 | SAU101175 | 5388 | SAU1c0031_orf_1p | 12213 |
| S1M10000044A09 | 3341 | SAU102292 | 5638 | SAU1c0038_orf_10p | 12368 |
| S1M10000044A11 | 3342 | SAU102602 | 5708 | SAU1c0032_orf_5p | 12249 |
| S1M10000044A12 | 3343 | SAU101791 | 5532 | SAU1c0032_orf_12p | 12216 |
| S1M10000044B01 | 3344 | SAU102268 | 5630 | SAU1c0032_orf_63p | 12252 |
| S1M10000044B02 | 3345 | SAU101968 | 5581 | SAU1c0028_orf_43p | 12187 |
| S1M10000044B05 | 3346 | SAU100690 | 5309 | #N/A | #N/A |
| S1M10000044B06 | 3347 | SAU100547 | 5290 | SAU1c0032_orf_3p | 12240 |
| S1M10000044B06 | 3347 | SAU102881 | 5740 | SAU1c0032_orf_4p | 12242 |
| S1M10000044B08 | 3348 | SAU101752 | 5522 | SAU1c0040_orf_85p | 12447 |
| S1M10000044B11 | 3349 | SAU101573 | 5485 | SAU1c0044_orf_212p | 12587 |
| S1M10000044B12 | 3350 | SAU201197 | 5806 | SAU2c0429_orf_2p | 12938 |
| S1M10000044C04 | 3351 | SAU101793 | 5534 | SAU1c0032_orf_14p | 12218 |
| S1M10000044C06 | 3352 | SAU101614 | 5494 | SAU1c0044_orf_9p | 12649 |
| S1M10000044C07 | 3353 | SAU100964 | 5363 | SAU1c0044_orf_86p | 12641 |
| S1M10000044C07 | 3353 | SAU100965 | 5364 | SAU1c0044_orf_87p | 12642 |
| S1M10000044C08 | 3354 | SAU102909 | 5743 | SAU1c0036_orf_16p | 12315 |
| S1M10000044C11 | 3355 | SAU101793 | 5534 | SAU1c0032_orf_14p | 12218 |
| S1M10000044C12 | 3356 | SAU102280 | 5632 | SAU1c0038_orf_3p | 12378 |
| S1M10000044D01 | 3357 | SAU100546 | 5289 | SAU1c0032_orf_2p | 12235 |
| S1M10000044D01 | 3357 | SAU102880 | 5739 | SAU1c0032_orf_1p | 12224 |
| S1M10000044D04 | 3358 | SAU101793 | 5534 | SAU1c0032_orf_14p | 12218 |
| S1M10000044D06 | 3359 | SAU101300 | 5415 | SAU1c0044_orf_113p | 12557 |
| S1M10000044D06 | 3359 | SAU101365 | 5432 | SAU1c0044_orf_112p | 12556 |
| S1M10000044D08 | 3360 | SAU102270 | 5631 | SAU1c0032_orf_65p | 12253 |
| S1M10000044D09 | 3361 | SAU100131 | 5232 | SAU1c0043_orf_156p | 12517 |
| S1M10000044D10 | 3362 | SAU201197 | 5806 | SAU2c0429_orf_2p | 12938 |
| S1M10000044D11 | 3363 | SAU101571 | 5483 | SAU1c0044_orf_210p | 12585 |
| S1M10000044D12 | 3364 | SAU102231 | 5614 | SAU1c0043_orf_18p | 12527 |
| S1M10000044D12 | 3364 | SAU102232 | 5615 | SAU1c0043_orf_19p | 12530 |
| S1M10000044E01 | 3365 | SAU101371 | 5435 | SAU1c0033_orf_7p | 12275 |
| S1M10000044E02 | 3366 | SAU102283 | 5634 | SAU1c0006_orf_1p | 12119 |
| S1M10000044E06 | 3367 | SAU201571 | 5824 | SAU2c0447_orf_17p | 12997 |
| S1M10000044E07 | 3368 | SAU301829 | 5902 | SAU3c1515_orf_7p | 13162 |
| S1M10000044E09 | 3369 | SAU101320 | 5420 | SAU1c0015_orf_16p | 12128 |
| S1M10000044E10 | 3370 | SAU100497 | 5280 | SAU1c0018_orf_3p | 12140 |
| S1M10000044E11 | 3371 | SAU101270 | 5410 | SAU1c0037_orf_89p | 12365 |
| S1M10000044E02 | 3372 | SAU101632 | 5499 | SAU1c0039_orf_3p | 12407 |
| S1M10000044F06 | 3373 | SAU101756 | 5524 | SAU1c0040_orf_82p | 12445 |
| S1M10000044F08 | 3374 | SAU101262 | 5406 | SAU1c0042_orf_113p | 12488 |
| S1M10000044F10 | 3375 | SAU101092 | 5381 | SAU1c0028_orf_9p | 12192 |
| S1M10000044F10 | 3375 | SAU202882 | 5855 | SAU2c0381_orf_3p | 12848 |
| S1M10000044G02 | 3376 | SAU102933 | 5744 | SAU1c0039_orf_62p | 12412 |
| S1M10000044G05 | 3377 | SAU101242 | 5404 | SAU1c0044_orf_18p | 12578 |
| S1M10000044G08 | 3378 | SAU102601 | 5707 | SAU1c0041_orf_46p | 12467 |
| S1M10000044G08 | 3378 | SAU102606 | 5711 | SAU1c0041_orf_50p | 12471 |
| S1M10000044G10 | 3379 | SAU101092 | 5381 | SAU1c0028_orf_9p | 12192 |
| S1M10000044G10 | 3379 | SAU202882 | 5855 | SAU2c0381_orf_3p | 12848 |
| S1M10000044G11 | 3380 | SAU101546 | 5475 | SAU1c0037_orf_133p | 12349 |
| S1M10000044H06 | 3381 | SAU100964 | 5363 | SAU1c0044_orf_86p | 12641 |
| S1M10000044H06 | 3381 | SAU100965 | 5364 | SAU1c0044_orf_87p | 12642 |
| S1M10000044H07 | 3382 | SAU100595 | 5294 | SAU1c0043_orf_62p | 12547 |
| S1M10000044H08 | 3383 | SAU101543 | 5473 | SAU1c0037_orf_130p | 12346 |
| S1M10000044H09 | 3384 | SAU100886 | 5349 | SAU1c0018_orf_16p | 12139 |
| S1M10000044H09 | 3384 | SAU100887 | 5350 | SAU1c0018_orf_15p | 12138 |
| S1M10000044H10 | 3385 | SAU101573 | 5485 | SAU1c0044_orf_212p | 12587 |
| S1M10000044H11 | 3386 | SAU102578 | 5701 | SAU1c0039_orf_61p | 12411 |
| S1M10000045A02 | 3387 | SAU100866 | 5344 | SAU1c0044_orf_100p | 12553 |
| S1M10000045A06 | 3388 | SAU102602 | 5708 | SAU1c0032_orf_5p | 12249 |
| S1M10000045A07 | 3389 | SAU102378 | 5653 | SAU1c0040_orf_61p | 12437 |
| S1M10000045A08 | 3390 | SAU102336 | 5646 | SAU1c0045_orf_146p | 12659 |
| S1M10000045A12 | 3391 | SAU201765 | 5833 | SAU2c0309_orf_5p | 12770 |
| S1M10000045B01 | 3392 | SAU101791 | 5532 | SAU1c0032_orf_12p | 12216 |
| S1M10000045B02 | 3393 | SAU100546 | 5289 | SAU1c0032_orf_2p | 12235 |
| S1M10000045B03 | 3394 | SAU200928 | 5798 | SAU2c0365_orf_5p | 12815 |
| S1M10000045B07 | 3395 | SAU101803 | 5543 | SAU1c0032_orf_23p | 12228 |
| S1M10000045B10 | 3396 | SAU200468 | 5781 | SAU2c0429_orf_19p | 12937 |
| S1M10000045B11 | 3397 | SAU101571 | 5483 | SAU1c0044_orf_210p | 12585 |
| S1M10000045B12 | 3398 | SAU101571 | 5483 | SAU1c0044_orf_210p | 12585 |
| S1M10000045C02 | 3399 | SAU100690 | 5309 | #N/A | #N/A |
| S1M10000045C03 | 3400 | SAU100887 | 5350 | SAU1c0018_orf_15p | 12138 |
| S1M10000045C04 | 3401 | SAU102286 | 5636 | SAU1c0038_orf_6p | 12393 |
| S1M10000045C04 | 3401 | SAU102287 | 5637 | SAU1c0038_orf_7p | 12398 |
| S1M10000045C05 | 3402 | SAU101571 | 5483 | SAU1c0044_orf_210p | 12585 |
| S1M10000045C07 | 3403 | SAU101573 | 5485 | SAU1c0044_orf_212p | 12587 |
| S1M10000045C09 | 3404 | SAU101744 | 5520 | SAU1c0037_orf_94p | 12367 |
| S1M10000045C09 | 3404 | SAU300191 | 5868 | SAU3c0672_orf_1p | 13037 |
| S1M10000045D01 | 3405 | SAU101893 | 5572 | SAU1c0034_orf_32p | 12282 |
| S1M10000045D03 | 3406 | SAU101599 | 5491 | SAU1c0041_orf_5p | 12478 |
| S1M10000045D07 | 3407 | SAU101491 | 5464 | SAU1c0025_orf_20p | 12165 |
| S1M10000045D08 | 3408 | SAU102117 | 5603 | SAU1c0027_orf_6p | 12181 |
| S1M10000045D09 | 3409 | SAU101572 | 5484 | SAU1c0044_orf_211p | 12586 |
| S1M10000045D10 | 3410 | SAU100866 | 5344 | SAU1c0044_orf_100p | 12553 |
| S1M10000045D11 | 3411 | SAU101492 | 5465 | SAU1c0025_orf_21p | 12166 |
| S1M10000045D11 | 3411 | SAU101493 | 5466 | SAU1c0025_orf_22p | 12167 |
| S1M10000045D12 | 3412 | SAU101800 | 5540 | SAU1c0032_orf_20p | 12225 |
| S1M10000045D12 | 3412 | SAU101801 | 5541 | #N/A | #N/A |
| S1M10000045E04 | 3413 | SAU102132 | 5605 | SAU1c0027_orf_19p | 12177 |
| S1M10000045E05 | 3414 | SAU101491 | 5464 | SAU1c0025_orf_20p | 12165 |
| S1M10000045E08 | 3415 | SAU201752 | 5832 | SAU2c0436_orf_19p | 12963 |
| S1M10000045E09 | 3416 | SAU101794 | 5535 | #N/A | #N/A |
| S1M10000045E10 | 3417 | SAU101756 | 5524 | SAU1c0040_orf_82p | 12445 |
| S1M10000045E11 | 3418 | SAU100970 | 5365 | SAU1c0043_orf_197p | 12529 |
| S1M10000045E12 | 3419 | SAU100547 | 5290 | SAU1c0032_orf_3p | 12240 |
| S1M10000045F04 | 3420 | SAU102241 | 5617 | SAU1c0043_orf_25p | 12539 |
| S1M10000045F05 | 3421 | SAU100114 | 5228 | SAU1c0043_orf_225p | 12535 |
| S1M10000045F08 | 3422 | SAU200657 | 5789 | #N/A | #N/A |
| S1M10000045F11 | 3423 | SAU102117 | 5603 | SAU1c0027_orf_6p | 12181 |
| S1M10000045F12 | 3424 | SAU101806 | 5546 | SAU1c0032_orf_25p | 12230 |
| S1M10000045G03 | 3425 | SAU102059 | 5597 | SAU1c0034_orf_51p | 12286 |
| S1M10000045G06 | 3426 | SAU101400 | 5444 | SAU1c0036_orf_35p | 12326 |
| S1M10000045G07 | 3427 | SAU101561 | 5479 | SAU1c0022_orf_4p | 12149 |
| S1M10000045G08 | 3428 | SAU100690 | 5309 | #N/A | #N/A |
| S1M10000045G10 | 3429 | SAU201571 | 5824 | SAU2c0447_orf_17p | 12997 |
| S1M10000045G12 | 3430 | SAU101400 | 5444 | SAU1c0036_orf_35p | 12326 |
| S1M10000045H06 | 3431 | SAU200928 | 5798 | SAU2c0365_orf_5p | 12815 |
| S1M10000045H10 | 3432 | SAU100414 | 5270 | SAU1c0022_orf_24p | 12148 |
| S1M10000045H11 | 3433 | SAU100414 | 5270 | SAU1c0022_orf_24p | 12148 |
| S1M10000046A03 | 3434 | SAU202731 | 5850 | #N/A | #N/A |
| S1M10000046A04 | 3435 | SAU100062 | 5225 | SAU1c0035_orf_98p | 12309 |
| S1M10000046A04 | 3435 | SAU100231 | 5245 | #N/A | #N/A |
| S1M10000046A06 | 3436 | SAU101383 | 5438 | SAU1c0022_orf_20p | 12147 |
| S1M10000046A08 | 3437 | SAU200994 | 5802 | SAU2c0428_orf_4p | 12935 |
| S1M10000046A09 | 3438 | SAU100315 | 5260 | SAU1c0037_orf_62p | 12358 |
| S1M10000046A11 | 3439 | SAU100432 | 5271 | SAU1c0040_orf_88p | 12450 |
| S1M10000046A11 | 3439 | SAU100433 | 5272 | SAU1c0040_orf_87p | 12449 |
| S1M10000046A12 | 3440 | SAU101814 | 5551 | SAU1c0032_orf_32p | 12237 |
| S1M10000046B01 | 3441 | SAU102334 | 5645 | SAU1c0045_orf_144p | 12658 |
| S1M10000046B03 | 3442 | SAU101039 | 5373 | SAU1c0043_orf_181p | 12522 |
| S1M10000046B04 | 3443 | SAU101797 | 5537 | SAU1c0032_orf_17p | 12221 |
| S1M10000046B05 | 3444 | SAU101156 | 5386 | SAU1c0036_orf_12p | 12311 |
| S1M10000046B07 | 3445 | SAU100866 | 5344 | SAU1c0044_orf_100p | 12553 |
| S1M10000046B08 | 3446 | SAU101365 | 5432 | SAU1c0044_orf_112p | 12556 |
| S1M10000046B09 | 3447 | SAU100866 | 5344 | SAU1c0044_orf_100p | 12553 |
| S1M10000046B11 | 3448 | SAU102541 | 5697 | SAU1c0045_orf_195p | 12668 |
| S1M10000046B12 | 3449 | SAU101400 | 5444 | SAU1c0036_orf_35p | 12326 |
| S1M10000046C02 | 3450 | SAU200601 | 5787 | #N/A | #N/A |
| S1M10000046C04 | 3451 | SAU100118 | 5229 | SAU1c0015_orf_13p | 12125 |
| S1M10000046C05 | 3452 | SAU101159 | 5387 | SAU1c0036_orf_46p | 12331 |
| S1M10000046C06 | 3453 | SAU102585 | 5703 | SAU1c0044_orf_289p | 12611 |
| S1M10000046C06 | 3453 | SAU201773 | 5834 | SAU2c0446_orf_4p | 12996 |
| S1M10000046C07 | 3454 | SAU102602 | 5708 | SAU1c0032_orf_5p | 12249 |
| S1M10000046C08 | 3455 | SAU100414 | 5270 | SAU1c0022_orf_24p | 12148 |
| S1M10000046C11 | 3456 | SAU102144 | 5608 | SAU1c0041_orf_15p | 12459 |
| S1M10000046C12 | 3457 | SAU100313 | 5259 | SAU1c0045_orf_153p | 12661 |
| S1M10000046C12 | 3457 | SAU100359 | 5264 | SAU1c0032_orf_35p | 12239 |
| S1M10000046D01 | 3458 | SAU100158 | 5238 | SAU1c0040_orf_80p | 12443 |
| S1M10000046D02 | 3459 | SAU102144 | 5608 | SAU1c0041_orf_15p | 12459 |
| S1M10000046D03 | 3460 | SAU101857 | 5560 | SAU1c0044_orf_156p | 12569 |
| S1M10000046D04 | 3461 | SAU102433 | 5668 | SAU1c0045_orf_37p | 12701 |
| S1M10000046D05 | 3462 | SAU102602 | 5708 | SAU1c0032_orf_5p | 12249 |
| S1M10000046D08 | 3463 | SAU101495 | 5467 | SAU1c0037_orf_65p | 12360 |
| S1M10000046D09 | 3464 | SAU100679 | 5305 | SAU1c0018_orf_14p | 12137 |
| S1M10000046D10 | 3465 | SAU101808 | 5548 | SAU1c0032_orf_27p | 12232 |
| S1M10000046D11 | 3466 | SAU100496 | 5279 | SAU1c0041_orf_83p | 12484 |
| S1M10000046D11 | 3466 | SAU301004 | 5882 | SAU3c1255_orf_1p | 13079 |
| S1M10000046D12 | 3467 | SAU100496 | 5279 | SAU1c0041_orf_83p | 12484 |
| S1M10000046D12 | 3467 | SAU301004 | 5882 | SAU3c1255_orf_1p | 13079 |
| S1M10000046E01 | 3468 | SAU101610 | 5492 | SAU1c0044_orf_5p | 12629 |
| S1M10000046E02 | 3469 | SAU101857 | 5560 | SAU1c0044_orf_156p | 12569 |
| S1M10000046E04 | 3470 | SAU101800 | 5540 | SAU1c0032_orf_20p | 12225 |
| S1M10000046E04 | 3470 | SAU101801 | 5541 | #N/A | #N/A |
| S1M10000046E07 | 3471 | SAU100521 | 5283 | SAU1c0044_orf_250p | 12600 |
| S1M10000046E08 | 3472 | SAU102283 | 5634 | SAU1c0006_orf_1p | 12119 |
| S1M10000046E10 | 3473 | SAU102283 | 5634 | SAU1c0006_orf_1p | 12119 |
| S1M10000046F01 | 3474 | SAU101028 | 5370 | SAU1c0043_orf_7p | 12552 |
| S1M10000046F02 | 3475 | SAU100546 | 5289 | SAU1c0032_orf_2p | 12235 |
| S1M10000046F02 | 3475 | SAU102880 | 5739 | SAU1c0032_orf_1p | 12224 |
| S1M10000046F05 | 3476 | SAU102671 | 5729 | SAU1c0024_orf_9p | 12161 |
| S1M10000046F06 | 3477 | SAU100702 | 5310 | SAU1c0029_orf_34p | 12196 |
| S1M10000046F06 | 3477 | SAU300825 | 5878 | SAU3c1171_orf_1p | 13068 |
| S1M10000046F08 | 3478 | SAU102297 | 5640 | SAU1c0045_orf_41p | 12704 |
| S1M10000046F09 | 3479 | SAU100517 | 5282 | #N/A | #N/A |
| S1M10000046F10 | 3480 | SAU102059 | 5597 | SAU1c0034_orf_51p | 12286 |
| S1M10000046F12 | 3481 | SAU101365 | 5432 | SAU1c0044_orf_112p | 12556 |
| S1M10000046G01 | 3482 | SAU200752 | 5795 | SAU2c0354_orf_5p | 12809 |
| S1M10000046G01 | 3482 | SAU300975 | 5880 | SAU3c1240_orf_3p | 13075 |
| S1M10000046G02 | 3483 | SAU101571 | 5483 | SAU1c0044_orf_210p | 12585 |
| S1M10000046G03 | 3484 | SAU100773 | 5326 | SAU1c0038_orf_39p | 12377 |
| S1M10000046G04 | 3485 | SAU100436 | 5273 | SAU1c0023_orf_20p | 12154 |
| S1M10000046G07 | 3486 | SAU101866 | 5564 | SAU1c0036_orf_21p | 12319 |
| S1M10000046G09 | 3487 | SAU102663 | 5727 | SAU1c0024_orf_2p | 12158 |
| S1M10000046G10 | 3488 | SAU101756 | 5524 | SAU1c0040_orf_82p | 12445 |
| S1M10000046H01 | 3489 | SAU101445 | 5452 | SAU1c0038_orf_47p | 12382 |
| S1M10000046H01 | 3489 | SAU101446 | 5453 | SAU1c0038_orf_48p | 12383 |
| S1M10000046H10 | 3490 | SAU200928 | 5798 | SAU2c0365_orf_5p | 12815 |
| S1M10000047A03 | 3491 | SAU100157 | 5237 | SAU1c0040_orf_81p | 12444 |
| S1M10000047A04 | 3492 | SAU300572 | 5873 | SAU3c1019_orf_1p | 13051 |
| S1M10000047A05 | 3493 | SAU101805 | 5545 | SAU1c0032_orf_24p | 12229 |
| S1M10000047A06 | 3494 | SAU201775 | 5835 | SAU2c0446_orf_4p | 12996 |
| S1M10000047A06 | 3494 | SAU301030 | 5883 | SAU3c1268_orf_1p | 13080 |
| S1M10000047A07 | 3495 | SAU101807 | 5547 | SAU1c0032_orf_26p | 12231 |
| S1M10000047A08 | 3496 | SAU102602 | 5708 | SAU1c0032_orf_5p | 12249 |
| S1M10000047A09 | 3497 | SAU101271 | 5411 | SAU1c0037_orf_90p | 12366 |
| S1M10000047A10 | 3498 | SAU100751 | 5321 | SAU1c0036_orf_59p | 12335 |
| S1M10000047A11 | 3499 | SAU100131 | 5232 | SAU1c0043_orf_156p | 12517 |
| S1M10000047A12 | 3500 | SAU100300 | 5253 | SAU1c0040_orf_90p | 12451 |
| S1M10000047B02 | 3501 | SAU101791 | 5532 | SAU1c0032_orf_12p | 12216 |
| S1M10000047B04 | 3502 | SAU101366 | 5433 | SAU1c0033_orf_2p | 12266 |
| S1M10000047B05 | 3503 | SAU101545 | 5474 | SAU1c0037_orf_132p | 12348 |
| S1M10000047B06 | 3504 | SAU200006 | 5770 | SAU2c0157_orf_1p | 12723 |
| S1M10000047B08 | 3505 | SAU101808 | 5548 | SAU1c0032_orf_27p | 12232 |
| S1M10000047B09 | 3506 | SAU100131 | 5232 | SAU1c0043_orf_156p | 12517 |
| S1M10000047B10 | 3507 | SAU101156 | 5386 | SAU1c0036_orf_12p | 12311 |
| S1M10000047B12 | 3508 | SAU101868 | 5565 | SAU1c0036_orf_23p | 12320 |
| S1M10000047C01 | 3509 | SAU100275 | 5252 | SAU1c0036_orf_15p | 12314 |
| S1M10000047C02 | 3510 | SAU101156 | 5386 | SAU1c0036_orf_12p | 12311 |
| S1M10000047C03 | 3511 | SAU200006 | 5770 | SAU2c0157_orf_1p | 12723 |
| S1M10000047C04 | 3512 | SAU101271 | 5411 | SAU1c0037_orf_90p | 12366 |
| S1M10000047C06 | 3513 | SAU101815 | 5552 | SAU1c0032_orf_33p | 12238 |
| S1M10000047C08 | 3514 | SAU101808 | 5548 | SAU1c0032_orf_27p | 12232 |
| S1M10000047C09 | 3515 | SAU101271 | 5411 | SAU1c0037_orf_90p | 12366 |
| S1M10000047C11 | 3516 | SAU201775 | 5835 | SAU2c0446_orf_4p | 12996 |
| S1M10000047C11 | 3516 | SAU301030 | 5883 | SAU3c1268_orf_1p | 13080 |
| S1M10000047C12 | 3517 | SAU101868 | 5565 | SAU1c0036_orf_23p | 12320 |
| S1M10000047D02 | 3518 | SAU101387 | 5440 | SAU1c0038_orf_52p | 12386 |
| S1M10000047D03 | 3519 | SAU101868 | 5565 | SAU1c0036_orf_23p | 12320 |
| S1M10000047D04 | 3520 | SAU100157 | 5237 | SAU1c0040_orf_81p | 12444 |
| S1M10000047D05 | 3521 | SAU101271 | 5411 | SAU1c0037_orf_90p | 12366 |
| S1M10000047D09 | 3522 | SAU100921 | 5355 | SAU1c0038_orf_76p | 12396 |
| S1M10000047D10 | 3523 | SAU201571 | 5824 | SAU2c0447_orf_17p | 12997 |
| S1M10000047D11 | 3524 | SAU103038 | 5757 | #N/A | #N/A |
| S1M10000047D12 | 3525 | SAU101175 | 5388 | SAU1c0031_orf_1p | 12213 |
| S1M10000047E01 | 3526 | SAU100158 | 5238 | SAU1c0040_orf_80p | 12443 |
| S1M10000047E02 | 3527 | SAU100131 | 5232 | SAU1c0043_orf_156p | 12517 |
| S1M10000047E03 | 3528 | SAU102452 | 5676 | SAU1c0045_orf_20p | 12674 |
| S1M10000047E04 | 3529 | SAU101996 | 5584 | SAU1c0040_orf_99p | 12456 |
| S1M10000047E05 | 3530 | SAU101815 | 5552 | SAU1c0032_orf_33p | 12238 |
| S1M10000047E06 | 3531 | SAU101807 | 5547 | SAU1c0032_orf_26p | 12231 |
| S1M10000047E08 | 3532 | SAU102200 | 5611 | SAU1c0045_orf_168p | 12665 |
| S1M10000047E09 | 3533 | SAU100810 | 5333 | SAU1c0037_orf_11p | 12343 |
| S1M10000047E10 | 3534 | SAU200928 | 5798 | SAU2c0365_orf_5p | 12815 |
| S1M10000047E11 | 3535 | SAU101156 | 5386 | SAU1c0036_orf_12p | 12311 |
| S1M10000047E12 | 3536 | SAU200928 | 5798 | SAU2c0365_orf_5p | 12815 |
| S1M10000047F02 | 3537 | SAU100158 | 5238 | SAU1c0040_orf_80p | 12443 |
| S1M10000047F03 | 3538 | SAU101242 | 5404 | SAU1c0044_orf_18p | 12578 |
| S1M10000047F04 | 3539 | SAU300572 | 5873 | SAU3c1019_orf_1p | 13051 |
| S1M10000047F05 | 3540 | SAU101271 | 5411 | SAU1c0037_orf_90p | 12366 |
| S1M10000047F06 | 3541 | SAU200928 | 5798 | SAU2c0365_orf_5p | 12815 |
| S1M10000047F07 | 3542 | SAU102602 | 5708 | SAU1c0032_orf_5p | 12249 |
| S1M10000047F08 | 3543 | SAU101242 | 5404 | SAU1c0044_orf_18p | 12578 |
| S1M10000047F09 | 3544 | SAU100157 | 5237 | SAU1c0040_orf_81p | 12444 |
| S1M10000047F10 | 3545 | SAU201571 | 5824 | SAU2c0447_orf_17p | 12997 |
| S1M10000047F11 | 3546 | SAU101805 | 5545 | SAU1c0032_orf_24p | 12229 |
| S1M10000047F12 | 3547 | SAU101808 | 5548 | SAU1c0032_orf_27p | 12232 |
| S1M10000047G01 | 3548 | SAU101369 | 5434 | SAU1c0033_orf_5p | 12274 |
| S1M10000047G02 | 3549 | SAU100141 | 5236 | SAU1c0032_orf_8p | 12259 |
| S1M10000047G04 | 3550 | SAU101341 | 5424 | SAU1c0044_orf_38p | 12618 |
| S1M10000047G05 | 3551 | SAU100684 | 5306 | SAU1c0044_orf_68p | 12632 |
| S1M10000047G05 | 3551 | SAU100685 | 5307 | SAU1c0044_orf_69p | 12633 |
| S1M10000047G06 | 3552 | SAU100141 | 5236 | SAU1c0032_orf_8p | 12259 |
| S1M10000047G07 | 3553 | SAU101798 | 5538 | SAU1c0032_orf_18p | 12222 |
| S1M10000047G08 | 3554 | SAU101028 | 5370 | SAU1c0043_orf_7p | 12552 |
| S1M10000047G09 | 3555 | SAU100810 | 5333 | SAU1c0037_orf_11p | 12343 |
| S1M10000047G10 | 3556 | SAU102607 | 5712 | SAU1c0041_orf_51p | 12472 |
| S1M10000047H03 | 3557 | SAU201571 | 5824 | SAU2c0447_orf_17p | 12997 |
| S1M10000047H04 | 3558 | SAU102200 | 5611 | SAU1c0045_orf_168p | 12665 |
| S1M10000047H05 | 3559 | SAU102452 | 5676 | SAU1c0045_orf_20p | 12674 |
| S1M10000047H06 | 3560 | SAU103038 | 5757 | #N/A | #N/A |
| S1M10000047H07 | 3561 | SAU200006 | 5770 | SAU2c0157_orf_1p | 12723 |
| S1M10000047H08 | 3562 | SAU101798 | 5538 | SAU1c0032_orf_18p | 12222 |
| S1M10000047H09 | 3563 | SAU102578 | 5701 | SAU1c0039_orf_61p | 12411 |
| S1M10000047H11 | 3564 | SAU101028 | 5370 | SAU1c0043_orf_7p | 12552 |
| S1M10000048A02 | 3565 | SAU201571 | 5824 | SAU2c0447_orf_17p | 12997 |
| S1M10000048A03 | 3566 | SAU100866 | 5344 | SAU1c0044_orf_100p | 12553 |
| S1M10000048A04 | 3567 | SAU103038 | 5757 | #N/A | #N/A |
| S1M10000048A05 | 3568 | SAU101868 | 5565 | SAU1c0036_orf_23p | 12320 |
| S1M10000048A06 | 3569 | SAU100157 | 5237 | SAU1c0040_orf_81p | 12444 |
| S1M10000048A07 | 3570 | SAU101156 | 5386 | SAU1c0036_orf_12p | 12311 |
| S1M10000048A09 | 3571 | SAU100158 | 5238 | SAU1c0040_orf_80p | 12443 |
| S1M10000048A10 | 3572 | SAU201571 | 5824 | SAU2c0447_orf_17p | 12997 |
| S1M10000048A11 | 3573 | SAU101807 | 5547 | SAU1c0032_orf_26p | 12231 |
| S1M10000048A12 | 3574 | SAU101271 | 5411 | SAU1c0037_orf_90p | 12366 |
| S1M10000048B02 | 3575 | SAU100608 | 5297 | SAU1c0034_orf_69p | 12293 |
| S1M10000048B05 | 3576 | SAU101028 | 5370 | SAU1c0043_orf_7p | 12552 |
| S1M10000048B08 | 3577 | SAU102452 | 5676 | SAU1c0045_orf_20p | 12674 |
| S1M10000048B10 | 3578 | SAU100158 | 5238 | SAU1c0040_orf_80p | 12443 |
| S1M10000048B11 | 3579 | SAU103038 | 5757 | #N/A | #N/A |
| S1M10000048B12 | 3580 | SAU200916 | 5797 | SAU2c0373_orf_4p | 12838 |
| S1M10000048B12 | 3580 | SAU301620 | 5899 | SAU3c1478_orf_2p | 13140 |
| S1M10000048C01 | 3581 | SAU101028 | 5370 | SAU1c0043_orf_7p | 12552 |
| S1M10000048C02 | 3582 | SAU301465 | 5896 | SAU3c1429_orf_4p | 13121 |
| S1M10000048C03 | 3583 | SAU102200 | 5611 | SAU1c0045_orf_168p | 12665 |
| S1M10000048C05 | 3584 | SAU300998 | 5881 | SAU3c1253_orf_3p | 13077 |
| S1M10000048C06 | 3585 | SAU100684 | 5306 | SAU1c0044_orf_68p | 12632 |
| S1M10000048C06 | 3585 | SAU100685 | 5307 | SAU1c0044_orf_69p | 12633 |
| S1M10000048C07 | 3586 | SAU102452 | 5676 | SAU1c0045_orf_20p | 12674 |
| S1M10000048C08 | 3587 | SAU101632 | 5499 | SAU1c0039_orf_3p | 12407 |
| S1M10000048C09 | 3588 | SAU101907 | 5574 | SAU1c0040_orf_79p | 12442 |
| S1M10000048C11 | 3589 | SAU101815 | 5552 | SAU1c0032_orf_33p | 12238 |
| S1M10000048D02 | 3590 | SAU100123 | 5230 | SAU1c0043_orf_189p | 12526 |
| S1M10000048D02 | 3590 | SAU102001 | 5586 | SAU1c0040_orf_102p | 12424 |
| S1M10000048D02 | 3590 | SAU103159 | 5762 | SAU1c0045_orf_204p | 12670 |
| S1M10000048D02 | 3590 | SAU201827 | 5837 | SAU2c0449_orf_21p | 13002 |
| S1M10000048D08 | 3591 | SAU300572 | 5873 | SAU3c1019_orf_1p | 13051 |
| S1M10000048D09 | 3592 | SAU100141 | 5236 | SAU1c0032_orf_8p | 12259 |
| S1M10000048D10 | 3593 | SAU302950 | 5914 | SAU3c1512_orf_12p | 13160 |
| S1M10000048D12 | 3594 | SAU102599 | 5706 | SAU1c0041_orf_45p | 12466 |
| S1M10000048D12 | 3594 | SAU103191 | 5765 | SAU1c00241_orf_44p | 12465 |
| S1M10000048E02 | 3595 | SAU101028 | 5370 | SAU1c0043_orf_7p | 12552 |
| S1M10000048E03 | 3596 | SAU102200 | 5611 | SAU1c0045_orf_168p | 12665 |
| S1M10000048E04 | 3597 | SAU101545 | 5474 | SAU1c0037_orf_132p | 12348 |
| S1M10000048E06 | 3598 | SAU200006 | 5770 | SAU2c0157_orf_1p | 12723 |
| S1M10000048E07 | 3599 | SAU100959 | 5359 | SAU1c0042_orf_102p | 12485 |
| S1M10000048E08 | 3600 | SAU101807 | 5547 | SAU1c0032_orf_26p | 12231 |
| S1M10000048E10 | 3601 | SAU302950 | 5914 | SAU3c1512_orf_12p | 13160 |
| S1M10000048F02 | 3602 | SAU101387 | 5440 | SAU1c0038_orf_52p | 12386 |
| S1M10000048F07 | 3603 | SAU101175 | 5388 | SAU1c0031_orf_1p | 12213 |
| S1M10000048F08 | 3604 | SAU100157 | 5237 | SAU1c0040_orf_81p | 12444 |
| S1M10000048F09 | 3605 | SAU101793 | 5534 | SAU1c0032_orf_14p | 12218 |
| S1M10000048F11 | 3606 | SAU202174 | 5845 | SAU2c0412_orf_3p | 12895 |
| S1M10000048F11 | 3606 | SAU301148 | 5888 | #N/A | #N/A |
| S1M10000048F12 | 3607 | SAU103038 | 5757 | #N/A | #N/A |
| S1M10000048G02 | 3608 | SAU102453 | 5677 | SAU1c0045_orf_19p | 12669 |
| S1M10000048G03 | 3609 | SAU200928 | 5798 | SAU2c0365_orf_5p | 12815 |
| S1M10000048G04 | 3610 | SAU102602 | 5708 | SAU1c0032_orf_5p | 12249 |
| S1M10000048G05 | 3611 | SAU101752 | 5522 | SAU1c0040_orf_85p | 12447 |
| S1M10000048G07 | 3612 | SAU102006 | 5589 | SAU1c0040_orf_107p | 12427 |
| S1M10000048G07 | 3612 | SAU102007 | 5590 | SAU1c0040_orf_108p | 12428 |
| S1M10000048G10 | 3613 | SAU101793 | 5534 | SAU1c0032_orf_14p | 12218 |
| S1M10000048G11 | 3614 | SAU200006 | 5770 | SAU2c0157_orf_1p | 12723 |
| S1M10000048H01 | 3615 | SAU100608 | 5297 | SAU1c0034_orf_69p | 12293 |
| S1M10000048H02 | 3616 | SAU100158 | 5238 | SAU1c0040_orf_80p | 12443 |
| S1M10000048H03 | 3617 | SAU101815 | 5552 | SAU1c0032_orf_33p | 12238 |
| S1M10000048H04 | 3618 | SAU102200 | 5611 | SAU1c0045_orf_168p | 12665 |
| S1M10000048H05 | 3619 | SAU100157 | 5237 | SAU1c0040_orf_81p | 12444 |
| S1M10000048H07 | 3620 | SAU100157 | 5237 | SAU1c0040_orf_81p | 12444 |
| S1M10000048H08 | 3621 | SAU100141 | 5236 | SAU1c0032_orf_8p | 12259 |
| S1M10000048H09 | 3622 | SAU100157 | 5237 | SAU1c0040_orf_81p | 12444 |
| S1M10000048H10 | 3623 | SAU101791 | 5532 | SAU1c0032_orf_12p | 12216 |
| S1M10000048H11 | 3624 | SAU101271 | 5411 | SAU1c0037_orf_90p | 12366 |
| K1M10000037D10 | 1077 | ECO100078 | 10023 | #N/A | #N/A |
| K1M10000002F02 | 1054 | ECO100252 | 10052 | #N/A | #N/A |
| K1M10000007F01 | 1057 | ECO100397 | 10064 | #N/A | #N/A |
| K1M10000007F01 | 1057 | ECO100398 | 10065 | #N/A | #N/A |
| K1M10000004F06 | 1056 | ECO100990 | 10120 | #N/A | #N/A |
| K1M10000019D06 | 1064 | ECO100990 | 10120 | #N/A | #N/A |
| K1M10000030C07 | 1070 | ECO102108 | 10214 | #N/A | #N/A |
| K1M10000044G05 | 1086 | ECO102262 | 10228 | #N/A | #N/A |
| K1M10000036G08 | 1076 | ECO102447 | 10247 | #N/A | #N/A |
| K1M10000033E01 | 1075 | ECO102539 | 10258 | #N/A | #N/A |
| K1M10000043D05 | 1081 | ECO102620 | 10266 | #N/A | #N/A |
| K1M10000045D10 | 1088 | ECO102620 | 10266 | #N/A | #N/A |
| K1M10000003C01 | 1055 | ECO103101 | 10315 | #N/A | #N/A |
| K1M10000030E07 | 1071 | ECO104120 | 10462 | #N/A | #N/A |
| K1M10000045A07 | 1087 | ECO104268 | 10475 | #N/A | #N/A |
| S4M10000020F05 | 3721 | ECO100449 | #N/A | #N/A | #N/A |
| S4M10000026D04 | 3742 | ECO100676 | #N/A | #N/A | #N/A |
| S4M10000014D07 | 3706 | ECO100757 | #N/A | #N/A | #N/A |
| S4M10000015B11 | 3708 | ECO100757 | #N/A | #N/A | #N/A |
| S4M10000016A02 | 3710 | ECO100757 | #N/A | #N/A | #N/A |
| S4M10000022E12 | 3725 | ECO100757 | #N/A | #N/A | #N/A |
| S4M10000026E12 | 3744 | ECO100757 | #N/A | #N/A | #N/A |
| S4M10000035E03 | 3764 | ECO100757 | #N/A | #N/A | #N/A |
| S4M10000008H10 | 3693 | ECO100758 | 10101 | #N/A | #N/A |
| S4M10000014B05 | 3704 | ECO100758 | 10101 | #N/A | #N/A |
| S4M10000014D07 | 3706 | ECO100758 | 10101 | #N/A | #N/A |
| S4M10000015B11 | 3708 | ECO100758 | 10101 | #N/A | #N/A |
| S4M10000015E09 | 3709 | ECO100758 | 10101 | #N/A | #N/A |
| S4M10000016A02 | 3710 | ECO100758 | 10101 | #N/A | #N/A |
| S4M10000022E12 | 3725 | ECO100758 | 10101 | #N/A | #N/A |
| S4M10000029B12 | 3747 | ECO100758 | 10101 | #N/A | #N/A |
| S4M10000020G10 | 3722 | ECO100796 | 10105 | #N/A | #N/A |
| S4M10000023F01 | 3728 | ECO101916 | #N/A | #N/A | #N/A |
| S4M10000014H02 | 3707 | ECO102028 | #N/A | #N/A | #N/A |
| S4M10000012B06 | 3700 | ECO102066 | #N/A | #N/A | #N/A |
| S4M10000035D01 | 3762 | ECO102066 | #N/A | #N/A | #N/A |
| S4M10000024C06 | 3730 | ECO102189 | 10224 | #N/A | #N/A |
| S4M10000006C05 | 3689 | ECO102282 | #N/A | #N/A | #N/A |
| S4M10000037H09 | 3772 | ECO102296 | #N/A | #N/A | #N/A |
| S4M10000030G11 | 3751 | ECO102302 | #N/A | #N/A | #N/A |
| S4M10000026C10 | 3741 | ECO102416 | 10245 | #N/A | #N/A |
| 34M10000026E06 | 3743 | ECO102416 | 10245 | #N/A | #N/A |
| S4M10000036F07 | 3768 | ECO102416 | 10245 | #N/A | #N/A |
| S4M10000034A02 | 3756 | ECO102526 | #N/A | #N/A | #N/A |
| S4M10000006F08 | 3690 | ECO102541 | 10259 | #N/A | #N/A |
| S4M10000002G08 | 3684 | ECO102730 | #N/A | #N/A | #N/A |
| S4M10000026C10 | 3741 | ECO102870 | #N/A | #N/A | #N/A |
| S4M10000026E06 | 3743 | ECO102870 | #N/A | #N/A | #N/A |
| S4M10000036F07 | 3768 | ECO102870 | #N/A | #N/A | #N/A |
| S4M10000034H05 | 3759 | ECO102900 | #N/A | #N/A | #N/A |
| S4M10000006A08 | 3688 | ECO102944 | #N/A | #N/A | #N/A |
| S4M10000014D04 | 3705 | ECO102986 | 10301 | #N/A | #N/A |
| S4M10000022D12 | 3724 | ECO103238 | 10354 | #N/A | #N/A |
| S4M10000033F08 | 3753 | ECO103238 | 10354 | #N/A | #N/A |
| S4M10000033G09 | 3755 | ECO103238 | 10354 | #N/A | #N/A |
| S4M10000001C01 | 3680 | ECO103265 | 10365 | #N/A | #N/A |
| S4M10000024B02 | 3729 | ECO103280 | #N/A | #N/A | #N/A |
| S4M10000020A04 | 3720 | ECO103461 | #N/A | #N/A | #N/A |
| S4M10000002B06 | 3681 | ECO103666 | #N/A | #N/A | #N/A |
| S4M10000019H06 | 3719 | ECO103738 | #N/A | #N/A | #N/A |
| S4M10000024H02 | 3736 | ECO103738 | #N/A | #N/A | #N/A |
| S4M10000030F07 | 3750 | ECO103738 | #N/A | #N/A | #N/A |
| S4M10000034H09 | 3760 | ECO103738 | #N/A | #N/A | #N/A |
| S4M10000032B12 | 3752 | ECO103935 | #N/A | #N/A | #N/A |
| S4M10000002B09 | 3682 | ECO103936 | #N/A | #N/A | #N/A |
| S4M10000037A10 | 3770 | ECO103951 | #N/A | #N/A | #N/A |
| S4M10000018D09 | 3711 | ECO104080 | #N/A | #N/A | #N/A |
| S4M10000035F09 | 3766 | EFA101301 | #N/A | EFA1c0040_orf_173p | #N/A |
| S4M10000035F09 | 3766 | EFA102170 | #N/A | EFA1c0040_orf_121p | #N/A |
| S4M10000001C01 | 3680 | EFA103268 | #N/A | EFA1c0010_orf_1p | 10479 |
| S4M10000036F07 | 3768 | HPY200334 | #N/A | #N/A | #N/A |
| S4M10000001C01 | 3680 | HPY201116 | 11570 | #N/A | #N/A |
| S4M10000037A10 | 3770 | KPN100467 | #N/A | KPN1c0583_orf_2p | #N/A |
| S4M10000030G11 | 3751 | KPN101078 | #N/A | KPN1c1190_orf_1p | #N/A |
| S4M10000024B02 | 3729 | KPN101160 | #N/A | KPN1c1224_orf_1p | #N/A |
| S4M10000032B12 | 3752 | KPN101846 | #N/A | KPN1c1681_orf_2p | #N/A |
| S4M10000006C05 | 3689 | KPN102011 | #N/A | KPN1c1862_orf_4p | #N/A |
| S4M10000035B01 | 3761 | KPN102014 | #N/A | KPN1c1786_orf_1p | 11654 |
| S4M10000012B06 | 3700 | KPN102524 | #N/A | #N/A | #N/A |
| S4M10000035D01 | 3762 | KPN102524 | #N/A | #N/A | #N/A |
| S4M10000002G04 | 3683 | KPN102558 | #N/A | KPN1c1982_orf_3p | #N/A |
| S4M10000002G08 | 3684 | KPN102558 | #N/A | KPN1c1982_orf_3p | #N/A |
| S4M10000008H10 | 3693 | KPN103640 | #N/A | KPN1c2761_orf_1p | #N/A |
| S4M10000014B05 | 3704 | KPN103640 | #N/A | KPN1c2761_orf_1p | #N/A |
| S4M10000014D07 | 3706 | KPN103640 | #N/A | KPN1c2761_orf_1p | #N/A |
| S4M10000015B11 | 3708 | KPN103640 | #N/A | KPN1c2761_orf_1p | #N/A |
| S4M10000015E09 | 3709 | KPN103640 | #N/A | KPN1c2761_orf_1p | #N/A |
| S4M10000016A02 | 3710 | KPN103640 | #N/A | KPN1c2761_orf_1p | #N/A |
| S4M10000022E12 | 3725 | KPN103640 | #N/A | KPN1c2761_orf_1p | #N/A |
| S4M10000026E12 | 3744 | KPN103640 | #N/A | KPN1c2761_orf_1p | #N/A |
| S4M10000035E03 | 3764 | KPN103640 | #N/A | KPN1c2761_orf_1p | #N/A |
| S4M10000008H10 | 3693 | KPN103641 | #N/A | KPN1c2761_orf_2p | 11705 |
| S4M10000014B05 | 3704 | KPN103641 | #N/A | KPN1c2761_orf_2p | 11705 |
| S4M10000014D07 | 3706 | KPN103641 | #N/A | KPN1c2761_orf_2p | 11705 |
| S4M10000015B11 | 3708 | KPN103641 | #N/A | KPN1c2761_orf_2p | 11705 |
| S4M10000015E09 | 3709 | KPN103641 | #N/A | KPN1c2761_orf_2p | 11705 |
| S4M10000016A02 | 3710 | KPN103641 | #N/A | KPN1c2761_orf_2p | 11705 |
| S4M10000022E12 | 3725 | KPN103641 | #N/A | KPN1c2761_orf_2p | 11705 |
| S4M10000029B12 | 3747 | KPN103641 | #N/A | KPN1c2761_orf_2p | 11705 |
| S4M10000035E03 | 3764 | KPN103641 | #N/A | KPN1c2761_orf_2p | 11705 |
| S4M10000026C10 | 3741 | KPN103871 | #N/A | KPN1c2844_orf_2p | #N/A |
| S4M10000026E06 | 3743 | KPN103871 | #N/A | KPN1c2844_orf_2p | #N/A |
| S4M10000036F07 | 3768 | KPN103871 | #N/A | KPN1c2844_orf_2p | #N/A |
| S4M10000019H06 | 3719 | KPN104321 | #N/A | KPN1c3011_orf_1p | #N/A |
| S4M10000024H02 | 3736 | KPN104321 | #N/A | KPN1c3011_orf_1p | #N/A |
| S4M10000030E07 | 3750 | KPN104321 | #N/A | KPN1c3011_orf_1p | #N/A |
| S4M10000034H09 | 3760 | KPN104321 | #N/A | KPN1c3011_orf_1p | #N/A |
| S4M10000035F02 | 3765 | KPN104321 | #N/A | KPN1c3011_orf_1p | #N/A |
| S4M10000002B06 | 3681 | KPN104608 | #N/A | KPN1c3070_orf_3p | #N/A |
| S4M10000018D09 | 3711 | KPN105957 | #N/A | KPN1c3587_orf_1p | #N/A |
| S4M10000024C06 | 3730 | KPN106468 | #N/A | KPN1c1186_orf_1p | 11638 |
| S4M10000009A05 | 3694 | KPN106681 | #N/A | #N/A | #N/A |
| S4M10000010D04 | 3696 | KPN106681 | #N/A | #N/A | #N/A |
| S4M10000011E08 | 3699 | KPN106681 | #N/A | #N/A | #N/A |
| S4M10000020A04 | 3720 | KPN106813 | #N/A | KPN1c2010_orf_1p | #N/A |
| S4M10000005G05 | 3685 | KPN106840 | #N/A | KPN1c2087_orf_1p | 11664 |
| S4M10000006F08 | 3690 | KPN106840 | #N/A | KPN1c2087_orf_1p | 11664 |
| S4M10000007G01 | 3691 | KPN106840 | #N/A | KPN1c2087_orf_1p | 11664 |
| S4M10000008C08 | 3692 | KPN106840 | #N/A | KPN1c2087_orf_1p | 11664 |
| S4M10000018E10 | 3712 | KPN106840 | #N/A | KPN1c2087_orf_1p | 11664 |
| S4M10000018E10 | 3713 | KPN106840 | #N/A | KPN1c2087_orf_1p | 11664 |
| S4M10000019G04 | 3717 | KPN106840 | #N/A | KPN1c2087_orf_1p | 11664 |
| S4M10000001C01 | 3680 | SAU101756 | #N/A | SAU1c0040_orf_82p | 12445 |
| S4M10000001C01 | 3680 | SAU200931 | #N/A | SAU2c0151_orf_1p | 12722 |
| S4M10000001C01 | 3680 | SPN102008 | #N/A | SPN1c0007_orf_92p | #N/A |
| S4M10000001C01 | 3680 | SPN202419 | #N/A | SPN2c0592_orf_1p | #N/A |
| S4M10000019H06 | 3719 | STY000068 | #N/A | STYc00048_orf_26p | #N/A |
| S4M10000024H02 | 3736 | STY000068 | #N/A | STYc00048_orf_26p | #N/A |
| S4M10000030F07 | 3750 | STY000068 | #N/A | STYc00048_orf_26p | #N/A |
| S4M10000034H09 | 3760 | STY000068 | #N/A | STYc00048_orf_26p | #N/A |
| S4M10000035F02 | 3765 | STY000068 | #N/A | STYc00048_orf_26p | #N/A |
| S4M10000026C10 | 3741 | STY000225 | #N/A | STYc00041_orf_40p | 13740 |
| S4M10000026E06 | 3743 | STY000225 | #N/A | STYc00041_orf_40p | 13740 |
| S4M10000036F07 | 3768 | STY000225 | #N/A | STYc00041_orf_40p | 13740 |
| S4M10000026D04 | 3742 | STY000244 | #N/A | STYc00041_orf_11p | #N/A |
| S4M10000034H05 | 3759 | STY000244 | #N/A | STYc00041_orf_11p | #N/A |
| S4M10000027E02 | 3746 | STY000409 | #N/A | STYc00053_orf_110p | #N/A |
| S4M10000025E02 | 3738 | STY000498 | #N/A | STYc00072_orf_46p | #N/A |
| S4M10000034A02 | 3756 | STY000498 | #N/A | STYc00072_orf_46p | #N/A |
| S4M10000034D06 | 3758 | STY000625 | #N/A | STYc00062_orf_63p | 13784 |
| S4M10000014D04 | 3705 | STY000737 | #N/A | STYc00054_orf_108p | 13759 |
| S4M10000013H02 | 3703 | STY000753 | #N/A | STYc00054_orf_91p | #N/A |
| S4M10000006A08 | 3688 | STY000817 | #N/A | STYc00054_orf_145p | #N/A |
| S4M10000036D07 | 3767 | STY000817 | #N/A | STYc00054_orf_145p | #N/A |
| S4M10000018D09 | 3711 | STY000848 | #N/A | STYc00101_orf_23p | #N/A |
| S4M10000014H02 | 3707 | STY000968 | #N/A | STYc00086_orf_86p | #N/A |
| S4M10000024G04 | 3734 | STY000986 | #N/A | #N/A | #N/A |
| S4M10000025H07 | 3740 | STY000986 | #N/A | #N/A | #N/A |
| S4M10000029D12 | 3748 | STY000986 | #N/A | #N/A | #N/A |
| S4M10000037A10 | 3770 | STY001009 | #N/A | STYc00080_orf_144p | #N/A |
| S4M10000035F09 | 3766 | STY001185 | #N/A | STYc00098_orf_2p | #N/A |
| S4M10000026D04 | 3742 | STY001220 | #N/A | STYc00123_orf_17p | #N/A |
| S4M10000022H06 | 3727 | STY001285 | #N/A | #N/A | #N/A |
| S4M10000030D03 | 3749 | STY001285 | #N/A | #N/A | #N/A |
| S4M10000037E10 | 3771 | STY001363 | #N/A | STYc00034_orf_126p | #N/A |
| S4M10000002B06 | 3681 | STY001380 | #N/A | STYc00119_orf_3p | #N/A |
| S4M10000011D08 | 3698 | STY001534 | #N/A | #N/A | #N/A |
| S4M10000024C11 | 3731 | STY001582 | #N/A | #N/A | #N/A |
| S4M10000025A11 | 3737 | STY001582 | #N/A | #N/A | #N/A |
| S4M10000035F09 | 3766 | STY001619 | #N/A | #N/A | #N/A |
| S4M10000022D12 | 3724 | STY001777 | #N/A | STYc00187_orf_4p | 13970 |
| S4M10000033F08 | 3753 | STY001777 | #N/A | STYc00187_orf_4p | 13970 |
| S4M10000033G09 | 3755 | STY001777 | #N/A | STYc00187_orf_4p | 13970 |
| S4M10000001C01 | 3680 | STY001790 | #N/A | STYc00187_orf_14p | 13967 |
| S4M10000026C10 | 3741 | STY001853 | #N/A | STYc00180_orf_22p | #N/A |
| S4M10000026E06 | 3743 | STY001853 | #N/A | STYc00180_orf_22p | #N/A |
| S4M10000036F07 | 3768 | STY001853 | #N/A | STYc00180_orf_22p | #N/A |
| S4M10000020A04 | 3720 | STY002064 | #N/A | STYc00074_orf_163p | #N/A |
| S4M10000020A04 | 3720 | STY002066 | #N/A | #N/A | #N/A |
| S4M10000024B02 | 3729 | STY002145 | #N/A | STYc00074_orf_17p | #N/A |
| S4M10000010B05 | 3695 | STY002525 | #N/A | STYc00114_orf_159p | #N/A |
| S4M10000012B12 | 3701 | STY002525 | #N/A | STYc00114_orf_159p | #N/A |
| S4M10000022D04 | 3723 | STY002525 | #N/A | STYc00114_orf_159p | #N/A |
| S4M10000022G07 | 3726 | STY002525 | #N/A | STYc00114_orf_159p | #N/A |
| S4M10000024G01 | 3733 | STY002525 | #N/A | STYc00114_orf_159p | #N/A |
| S4M10000024G09 | 3735 | STY002525 | #N/A | STYc00114_orf_159p | #N/A |
| S4M10000025E05 | 3739 | STY002525 | #N/A | STYc00114_orf_159p | #N/A |
| S4M10000027C10 | 3745 | STY002525 | #N/A | STYc00114_orf_159p | #N/A |
| S4M10000034A09 | 3757 | STY002525 | #N/A | STYc00114_orf_159p | #N/A |
| S4M10000037H09 | 3772 | STY002590 | #N/A | STYc00114_orf_104p | #N/A |
| S4M10000002G04 | 3683 | STY002623 | #N/A | STYc00114_orf_90p | #N/A |
| S4M10000002G08 | 3684 | STY002623 | #N/A | STYc00114_orf_90p | #N/A |
| S4M10000033G05 | 3754 | STY002638 | #N/A | STYc00114_orf_15p | 13870 |
| S4M10000006A06 | 3687 | STY002672 | #N/A | STYc00114_orf_136p | #N/A |
| S4M10000010H04 | 3697 | STY002711 | #N/A | STYc00223_orf_1p | #N/A |
| S4M10000005H02 | 3686 | STY002738 | #N/A | STYc00223_orf_17p | #N/A |
| S4M10000012B06 | 3700 | STY002826 | #N/A | STYc00152_orf_12p | #N/A |
| S4M10000035D01 | 3762 | STY002826 | #N/A | STYc00152_orf_12p | #N/A |
| S4M10000018G03 | 3714 | STY002889 | #N/A | #N/A | #N/A |
| S4M10000018H04 | 3715 | STY002889 | #N/A | #N/A | #N/A |
| S4M10000019F05 | 3716 | STY002889 | #N/A | #N/A | #N/A |
| S4M10000019G05 | 3718 | STY002889 | #N/A | #N/A | #N/A |
| S4M10000037E10 | 3771 | STY002959 | #N/A | STYc00215_orf_11p | 14011 |
| S4M10000002B09 | 3682 | STY003082 | #N/A | STYc00238_orf_12p | #N/A |
| S4M10000032B12 | 3752 | STY003084 | #N/A | STYc00238_orf_13p | #N/A |
| S4M10000024C06 | 3730 | STY003375 | #N/A | STYc00183_orf_19p | 13957 |
| S4M10000012D02 | 3702 | STY003377 | #N/A | #N/A | #N/A |
| S4M10000030G11 | 3751 | STY003384 | #N/A | STYc00183_orf_130p | #N/A |
| S4M10000006F08 | 3690 | STY003460 | #N/A | STYc00339_orf_20p | 14087 |
| S4M10000024F08 | 3732 | STY003664 | #N/A | #N/A | #N/A |
| S4M10000020G10 | 3722 | STY004048 | #N/A | STYc00207_orf_161p | 13999 |
| S4M10000008H10 | 3693 | STY004152 | #N/A | STYc00207_orf_194p | 14003 |
| S4M10000014B05 | 3704 | STY004152 | #N/A | STYc00207_orf_194p | 14003 |
| S4M10000015E09 | 3709 | STY004152 | #N/A | STYc00207_orf_194p | 14003 |
| S4M10000016A02 | 3710 | STY004152 | #N/A | STYc00207_orf_194p | 14003 |
| S4M10000022E12 | 3725 | STY004152 | #N/A | STYc00207_orf_194p | 14003 |
| S4M10000029B12 | 3747 | STY004152 | #N/A | STYc00207_orf_194p | 14003 |
| S4M10000035E03 | 3764 | STY004152 | #N/A | STYc00207_orf_194p | 14003 |
| S4M10000008H10 | 3693 | STY004154 | #N/A | STYc00207_orf_195p | #N/A |
| S4M10000014B05 | 3704 | STY004154 | #N/A | STYc00207_orf_195p | #N/A |
| S4M10000014D07 | 3706 | STY004154 | #N/A | STYc00207_orf_195p | #N/A |
| S4M10000015B11 | 3708 | STY004154 | #N/A | STYc00207_orf_195p | #N/A |
| S4M10000015E09 | 3709 | STY004154 | #N/A | STYc00207_orf_195p | #N/A |
| S4M10000016A02 | 3710 | STY004154 | #N/A | STYc00207_orf_195p | #N/A |
| S4M10000022E12 | 3725 | STY004154 | #N/A | STYc00207_orf_195p | #N/A |
| S4M10000026E12 | 3744 | STY004154 | #N/A | STYc00207_orf_195p | #N/A |
| S4M10000035E03 | 3764 | STY004154 | #N/A | STYc00207_orf_195p | #N/A |
| S4M10000005G05 | 3685 | STY004239 | #N/A | #N/A | #N/A |
| S4M10000007G01 | 3691 | STY004239 | #N/A | #N/A | #N/A |
| S4M10000008C08 | 3692 | STY004239 | #N/A | #N/A | #N/A |
| S4M10000018E10 | 3712 | STY004239 | #N/A | #N/A | #N/A |
| S4M10000018F10 | 3713 | STY004239 | #N/A | #N/A | #N/A |
| S4M10000019G04 | 3717 | STY004239 | #N/A | #N/A | #N/A |
| S4M10000037A04 | 3769 | STY005016 | #N/A | #N/A | #N/A |
| K1M10000007F01 | 1057 | SAU100968 | #N/A | SAU1c0044_orf_90p | 12643 |
| K1M10000007E01 | 1057 | SAU201145 | #N/A | SAU2c0405_orf_7p | 12884 |
| K1M10000007F01 | 1057 | SPN101971 | #N/A | SPN1c0209_orf_54p | #N/A |
| K1M10000007F01 | 1057 | SPN201024 | #N/A | SPN2c0417_orf_4p | #N/A |
| K1M10000003C01 | 1055 | STY000773 | #N/A | STYc00054_orf_16p | 13764 |
| K1M10000023E09 | 1068 | STY000886 | #N/A | #N/A | #N/A |
| K1M10000023E10 | 1069 | STY000886 | #N/A | #N/A | #N/A |
| K1M10000007F01 | 1057 | STY001430 | #N/A | STYc00148_orf_11p | 13915 |
| K1M10000007F01 | 1057 | STY001433 | #N/A | STYc00148_orf_12p | 13916 |
| K1M10000036G08 | 1076 | STY001867 | #N/A | STYc00180_orf_50p | 13948 |
| K1M10000030C07 | 1070 | STY002768 | #N/A | #N/A | #N/A |
| K1M10000037D10 | 1077 | STY002995 | #N/A | STYc00215_orf_67p | 14018 |
| K1M10000044G05 | 1086 | STY003357 | #N/A | STYc00183_orf_91p | 13963 |

TABLE IC

| PathoSeq Gene Locus | Nucleotide SeqID | Protein SeqID |
|---|---|---|
| EFA100001 | 3806 | 4861 |
| EFA100023 | 3807 | 4862 |
| EFA100065 | 3808 | 4863 |
| EFA100151 | 3809 | 4864 |
| EFA100157 | 3810 | 4865 |
| EFA100165 | 3811 | 4866 |
| EFA100190 | 3812 | 4867 |
| EFA100194 | 3813 | 4868 |
| EFA100200 | 3814 | 4869 |
| EFA100210 | 3815 | 4870 |
| EFA100211 | 3816 | 4871 |
| EFA100289 | 3817 | 4872 |
| EFA100295 | 3818 | 4873 |
| EFA100312 | 3819 | 4874 |
| EFA100329 | 3820 | 4875 |
| EFA100394 | 3821 | 4876 |
| EFA100397 | 3822 | 4877 |
| EFA100399 | 3823 | 4878 |
| EFA100426 | 3824 | 4879 |
| EFA100478 | 3825 | 4880 |
| EFA100615 | 3826 | 4881 |
| EFA100617 | 3827 | 4882 |
| EFA100641 | 3828 | 4883 |
| EFA100642 | 3829 | 4884 |
| EFA100668 | 3830 | 4885 |
| EFA100689 | 3831 | 4886 |
| EFA100704 | 3832 | 4887 |
| EFA100739 | 3833 | 4888 |
| EFA100740 | 3834 | 4889 |
| EFA100741 | 3835 | 4890 |
| EFA100742 | 3836 | 4891 |
| EFA100748 | 3837 | 4892 |
| EFA100756 | 3838 | 4893 |
| EFA100757 | 3839 | 4894 |
| EFA100783 | 3840 | 4895 |
| EFA100795 | 3841 | 4896 |
| EFA100798 | 3842 | 4897 |
| EFA100811 | 3843 | 4898 |
| EFA100870 | 3844 | 4899 |
| EFA100914 | 3845 | 4900 |
| EFA100919 | 3846 | 4901 |
| EFA100955 | 3847 | 4902 |
| EFA100970 | 3848 | 4903 |
| EFA100978 | 3849 | 4904 |
| EFA100991 | 3850 | 4905 |
| EFA101022 | 3851 | 4906 |
| EFA101060 | 3852 | 4907 |
| EFA101079 | 3853 | 4908 |
| EFA101080 | 3854 | 4909 |
| EFA101086 | 3855 | 4910 |
| EFA101120 | 3856 | 4911 |
| EFA101121 | 3857 | 4912 |
| EFA101123 | 3858 | 4913 |
| EFA101141 | 3859 | 4914 |
| EFA101150 | 3860 | 4915 |
| EFA101159 | 3861 | 4916 |
| EFA101160 | 3862 | 4917 |
| EFA101161 | 3863 | 4918 |
| EFA101162 | 3864 | 4919 |
| EFA101163 | 3865 | 4920 |
| EFA101164 | 3866 | 4921 |
| EFA101165 | 3867 | 4922 |
| EFA101169 | 3868 | 4923 |
| EFA101253 | 3869 | 4924 |
| EFA101257 | 3870 | 4925 |
| EFA101258 | 3871 | 4926 |
| EFA101322 | 3872 | 4927 |
| EFA101339 | 3873 | 4928 |
| EFA101340 | 3874 | 4929 |
| EFA101354 | 3875 | 4930 |
| EFA101370 | 3876 | 4931 |
| EFA101403 | 3877 | 4932 |
| EFA101404 | 3878 | 4933 |
| EFA101409 | 3879 | 4934 |
| EFA101410 | 3880 | 4935 |
| EFA101411 | 3881 | 4936 |
| EFA101412 | 3882 | 4937 |
| EFA101413 | 3883 | 4938 |
| EFA101414 | 3884 | 4939 |
| EFA101415 | 3885 | 4940 |
| EFA101416 | 3886 | 4941 |
| EFA101417 | 3887 | 4942 |
| EFA101424 | 3888 | 4943 |
| EFA101425 | 3889 | 4944 |
| EFA101477 | 3890 | 4945 |
| EFA101536 | 3891 | 4946 |
| EFA101540 | 3892 | 4947 |
| EFA101541 | 3893 | 4948 |
| EFA101583 | 3894 | 4949 |
| EFA101670 | 3895 | 4950 |
| EFA101682 | 3896 | 4951 |
| EFA101685 | 3897 | 4952 |
| EFA101686 | 3898 | 4953 |
| EFA101695 | 3899 | 4954 |
| EFA101736 | 3900 | 4955 |
| EFA101737 | 3901 | 4956 |
| EFA101753 | 3902 | 4957 |
| EFA101765 | 3903 | 4958 |
| EFA101790 | 3904 | 4959 |
| EFA101791 | 3905 | 4960 |
| EFA101792 | 3906 | 4961 |
| EFA101795 | 3907 | 4962 |
| EFA101797 | 3908 | 4963 |
| EFA101799 | 3909 | 4964 |
| EFA101833 | 3910 | 4965 |
| EFA101868 | 3911 | 4966 |
| EFA101872 | 3912 | 4967 |
| EFA101873 | 3913 | 4968 |
| EFA101892 | 3914 | 4969 |
| EFA101924 | 3915 | 4970 |
| EFA101925 | 3916 | 4971 |
| EFA101963 | 3917 | 4972 |
| EFA102006 | 3918 | 4973 |
| EFA102022 | 3919 | 4974 |
| EFA102023 | 3920 | 4975 |
| EFA102051 | 3921 | 4976 |
| EFA102091 | 3922 | 4977 |
| EFA102110 | 3923 | 4978 |
| EFA102183 | 3924 | 4979 |
| EFA102185 | 3925 | 4980 |
| EFA102186 | 3926 | 4981 |
| EFA102201 | 3927 | 4982 |
| EFA102205 | 3928 | 4983 |
| EFA102253 | 3929 | 4984 |
| EFA102282 | 3930 | 4985 |
| EFA102326 | 3931 | 4986 |
| EFA102338 | 3932 | 4987 |
| EFA102350 | 3933 | 4988 |
| EFA102351 | 3934 | 4989 |
| EFA102352 | 3935 | 4990 |
| EFA102353 | 3936 | 4991 |
| EFA102389 | 3937 | 4992 |
| EFA102453 | 3938 | 4993 |
| EFA102501 | 3939 | 4994 |
| EFA102502 | 3940 | 4995 |
| EFA102503 | 3941 | 4996 |
| EFA102518 | 3942 | 4997 |
| EFA102541 | 3943 | 4998 |
| EFA102542 | 3944 | 4999 |
| EFA102549 | 3945 | 5000 |
| EFA102551 | 3946 | 5001 |
| EFA102554 | 3947 | 5002 |
| EFA102655 | 3948 | 5003 |
| EFA102656 | 3949 | 5004 |
| EFA102698 | 3950 | 5005 |
| EFA102728 | 3951 | 5006 |
| EFA102736 | 3952 | 5007 |
| EFA102764 | 3953 | 5008 |
| EFA102774 | 3954 | 5009 |
| EFA102780 | 3955 | 5010 |
| EFA102788 | 3956 | 5011 |
| EFA102802 | 3957 | 5012 |
| EFA102813 | 3958 | 5013 |
| EFA102915 | 3959 | 5014 |
| EFA103021 | 3960 | 5015 |
| EFA103033 | 3961 | 5016 |
| EFA103038 | 3962 | 5017 |
| EFA103039 | 3963 | 5018 |
| EFA103062 | 3964 | 5019 |
| EFA103081 | 3965 | 5020 |
| EFA103174 | 3966 | 5021 |
| EFA103210 | 3967 | 5022 |
| EFA103268 | 3968 | 5023 |
| EFA103295 | 3969 | 5024 |
| EFA103348 | 3970 | 5025 |
| EFA103365 | 3971 | 5026 |
| EFA103375 | 3972 | 5027 |
| EFA103504 | 3973 | 5028 |
| EFA103508 | 3974 | 5029 |
| EFA103571 | 3975 | 5030 |
| EFA103786 | 3976 | 5031 |
| KPN100432 | 3977 | 5032 |
| KPN100854 | 3978 | 5033 |
| KPN101022 | 3979 | 5034 |
| KPN101026 | 3980 | 5035 |
| KPN101729 | 3981 | 5036 |
| KPN101750 | 3982 | 5037 |
| KPN102057 | 3983 | 5038 |
| KPN102638 | 3984 | 5039 |
| KPN103882 | 3985 | 5040 |
| KPN104183 | 3986 | 5041 |
| KPN104281 | 3987 | 5042 |
| KPN104430 | 3988 | 5043 |
| KPN104538 | 3989 | 5044 |
| KPN104716 | 3990 | 5045 |
| KPN105722 | 3991 | 5046 |
| KPN105779 | 3992 | 5047 |
| KPN106044 | 3993 | 5048 |
| KPN106659 | 3994 | 5049 |
| KPN106840 | 3995 | 5050 |
| KPN107626 | 3996 | 5051 |
| KPN107776 | 3997 | 5052 |
| PA0028 | 3998 | 5053 |
| PA0120 | 3999 | 5054 |
| PA0129 | 4000 | 5055 |
| PA0141 | 4001 | 5056 |
| PA0221 | 4002 | 5057 |
| PA0265 | 4003 | 5058 |
| PA0321 | 4004 | 5059 |
| PA0337 | 4005 | 5060 |
| PA0353 | 4006 | 5061 |
| PA0378 | 4007 | 5062 |
| PA0401 | 4008 | 5063 |
| PA0413 | 4009 | 5064 |
| PA0414 | 4010 | 5065 |
| PA0419 | 4011 | 5066 |
| PA0423 | 4012 | 5067 |
| PA0469 | 4013 | 5068 |
| PA0472 | 4014 | 5069 |
| PA0506 | 4015 | 5070 |
| PA0600 | 4016 | 5071 |
| PA0642 | 4017 | 5072 |
| PA0650 | 4018 | 5073 |
| PA0715 | 4019 | 5074 |
| PA0788 | 4020 | 5075 |
| PA0882 | 4021 | 5076 |
| PA0934 | 4022 | 5077 |
| PA0938 | 4023 | 5078 |
| PA1019 | 4024 | 5079 |
| PA1072 | 4025 | 5080 |
| PA1115 | 4026 | 5081 |
| PA1270 | 4027 | 5082 |
| PA1301 | 4028 | 5083 |
| PA1360 | 4029 | 5084 |
| PA1365 | 4030 | 5085 |
| PA1398 | 4031 | 5086 |
| PA1462 | 4032 | 5087 |
| PA1493 | 4033 | 5088 |
| PA1547 | 4034 | 5089 |
| PA1636 | 4035 | 5090 |
| PA1684 | 4036 | 5091 |
| PA1868 | 4037 | 5092 |
| PA1876 | 4038 | 5093 |
| PA1918 | 4039 | 5094 |
| PA1986 | 4040 | 5095 |
| PA2009 | 4041 | 5096 |
| PA2083 | 4042 | 5097 |
| PA2101 | 4043 | 5098 |
| PA2108 | 4044 | 5099 |
| PA2128 | 4045 | 5100 |
| PA2147 | 4046 | 5101 |
| PA2196 | 4047 | 5102 |
| PA2197 | 4048 | 5103 |
| PA2222 | 4049 | 5104 |
| PA2313 | 4050 | 5105 |
| PA2398 | 4051 | 5106 |
| PA2424 | 4052 | 5107 |
| PA2461 | 4053 | 5108 |
| PA2470 | 4054 | 5109 |
| PA2488 | 4055 | 5110 |
| PA2494 | 4056 | 5111 |
| PA2584 | 4057 | 5112 |
| PA2594 | 4058 | 5113 |
| PA2634 | 4059 | 5114 |
| PA2641 | 4060 | 5115 |
| PA2671 | 4061 | 5116 |
| PA2680 | 4062 | 5117 |
| PA2684 | 4063 | 5118 |
| PA2726 | 4064 | 5119 |
| PA2742 | 4065 | 5120 |
| PA3006 | 4066 | 5121 |
| PA3011 | 4067 | 5122 |
| PA3013 | 4068 | 5123 |
| PA3041 | 4069 | 5124 |
| PA3048 | 4070 | 5125 |
| PA3068 | 4071 | 5126 |
| PA3121 | 4072 | 5127 |
| PA3153 | 4073 | 5128 |
| PA3154 | 4074 | 5129 |
| PA3160 | 4075 | 5130 |
| PA3279 | 4076 | 5131 |
| PA3280 | 4077 | 5132 |
| PA3374 | 4078 | 5133 |
| PA3479 | 4079 | 5134 |
| PA3484 | 4080 | 5135 |
| PA3522 | 4081 | 5136 |
| PA3643 | 4082 | 5137 |
| PA3703 | 4083 | 5138 |
| PA3709 | 4084 | 5139 |
| PA3716 | 4085 | 5140 |
| PA3764 | 4086 | 5141 |
| PA3845 | 4087 | 5142 |
| PA3866 | 4088 | 5143 |
| PA3876 | 4089 | 5144 |
| PA3877 | 4090 | 5145 |
| PA3931 | 4091 | 5146 |
| PA3984 | 4092 | 5147 |
| PA4024 | 4093 | 5148 |
| PA4027 | 4094 | 5149 |
| PA4037 | 4095 | 5150 |
| PA4067 | 4096 | 5151 |
| PA4070 | 4097 | 5152 |
| PA4081 | 4098 | 5153 |
| PA4105 | 4099 | 5154 |
| PA4124 | 4100 | 5155 |
| PA4125 | 4101 | 5156 |
| PA4158 | 4102 | 5157 |
| PA4237 | 4103 | 5158 |
| PA4242 | 4104 | 5159 |
| PA4244 | 4105 | 5160 |
| PA4245 | 4106 | 5161 |
| PA4246 | 4107 | 5162 |
| PA4247 | 4108 | 5163 |
| PA4248 | 4109 | 5164 |
| PA4249 | 4110 | 5165 |
| PA4250 | 4111 | 5166 |
| PA4251 | 4112 | 5167 |
| PA4252 | 4113 | 5168 |
| PA4253 | 4114 | 5169 |
| PA4254 | 4115 | 5170 |
| PA4256 | 4116 | 5171 |
| PA4257 | 4117 | 5172 |
| PA4258 | 4118 | 5173 |
| PA4259 | 4119 | 5174 |
| PA4262 | 4120 | 5175 |
| PA4263 | 4121 | 5176 |
| PA4264 | 4122 | 5177 |
| PA4268 | 4123 | 5178 |
| PA4269 | 4124 | 5179 |
| PA4271 | 4125 | 5180 |
| PA4272 | 4126 | 5181 |
| PA4316 | 4127 | 5182 |
| PA4332 | 4128 | 5183 |
| PA4347 | 4129 | 5184 |
| PA4363 | 4130 | 5185 |
| PA4375 | 4131 | 5186 |
| PA4413 | 4132 | 5187 |
| PA4433 | 4133 | 5188 |
| PA4473 | 4134 | 5189 |
| PA4506 | 4135 | 5190 |
| PA4512 | 4136 | 5191 |
| PA4542 | 4137 | 5192 |
| PA4576 | 4138 | 5193 |
| PA4598 | 4139 | 5194 |
| PA4665 | 4140 | 5195 |
| PA4681 | 4141 | 5196 |
| PA4709 | 4142 | 5197 |
| PA4744 | 4143 | 5198 |
| PA4771 | 4144 | 5199 |
| PA4888 | 4145 | 5200 |
| PA4942 | 4146 | 5201 |
| PA4997 | 4147 | 5202 |
| PA5030 | 4148 | 5203 |
| PA5076 | 4149 | 5204 |
| PA5088 | 4150 | 5205 |
| PA5193 | 4151 | 5206 |
| PA5199 | 4152 | 5207 |
| PA5207 | 4153 | 5208 |
| PA5209 | 4154 | 5209 |
| PA5248 | 4155 | 5210 |
| PA5299 | 4156 | 5211 |
| PA5316 | 4157 | 5212 |
| PA5388 | 4158 | 5213 |
| PA5393 | 4159 | 5214 |
| PA5436 | 4160 | 5215 |
| PA5443 | 4161 | 5216 |
| PA5490 | 4162 | 5217 |
| PA5493 | 4163 | 5218 |
| PA5507 | 4164 | 5219 |
| PA5567 | 4165 | 5220 |
| SAU100040 | 4166 | 5221 |
| SAU100053 | 4167 | 5222 |
| SAU100056 | 4168 | 5223 |
| SAU100059 | 4169 | 5224 |
| SAU100062 | 4170 | 5225 |
| SAU100077 | 4171 | 5226 |
| SAU100112 | 4172 | 5227 |
| SAU100114 | 4173 | 5228 |
| SAU100118 | 4174 | 5229 |
| SAU100123 | 4175 | 5230 |
| SAU100128 | 4176 | 5231 |
| SAU100131 | 4177 | 5232 |
| SAU100133 | 4178 | 5233 |
| SAU100139 | 4179 | 5234 |
| SAU100140 | 4180 | 5235 |
| SAU100141 | 4181 | 5236 |
| SAU100157 | 4182 | 5237 |
| SAU100158 | 4183 | 5238 |
| SAU100162 | 4184 | 5239 |
| SAU100175 | 4185 | 5240 |
| SAU100182 | 4186 | 5241 |
| SAU100186 | 4187 | 5242 |
| SAU100198 | 4188 | 5243 |
| SAU100227 | 4189 | 5244 |
| SAU100231 | 4190 | 5245 |
| SAU100242 | 4191 | 5246 |
| SAU100246 | 4192 | 5247 |
| SAU100251 | 4193 | 5248 |
| SAU100265 | 4194 | 5249 |
| SAU100266 | 4195 | 5250 |
| SAU100272 | 4196 | 5251 |
| SAU100275 | 4197 | 5252 |
| SAU100300 | 4198 | 5253 |
| SAU100301 | 4199 | 5254 |
| SAU100302 | 4200 | 5255 |
| SAU100305 | 4201 | 5256 |
| SAU100307 | 4202 | 5257 |
| SAU100308 | 4203 | 5258 |
| SAU100313 | 4204 | 5259 |
| SAU100315 | 4205 | 5260 |
| SAU100323 | 4206 | 5261 |
| SAU100347 | 4207 | 5262 |
| SAU100355 | 4208 | 5263 |
| SAU100359 | 4209 | 5264 |
| SAU100381 | 4210 | 5265 |
| SAU100389 | 4211 | 5266 |
| SAU100390 | 4212 | 5267 |
| SAU100401 | 4213 | 5268 |
| SAU100412 | 4214 | 5269 |
| SAU100414 | 4215 | 5270 |
| SAU100432 | 4216 | 5271 |
| SAU100433 | 4217 | 5272 |
| SAU100436 | 4218 | 5273 |
| SAU100443 | 4219 | 5274 |
| SAU100444 | 4220 | 5275 |
| SAU100475 | 4221 | 5276 |
| SAU100478 | 4222 | 5277 |
| SAU100489 | 4223 | 5278 |
| SAU100496 | 4224 | 5279 |
| SAU100497 | 4225 | 5280 |
| SAU100514 | 4226 | 5281 |
| SAU100517 | 4227 | 5282 |
| SAU100521 | 4228 | 5283 |
| SAU100522 | 4229 | 5284 |
| SAU100527 | 4230 | 5285 |
| SAU100528 | 4231 | 5286 |
| SAU100532 | 4232 | 5287 |
| SAU100542 | 4233 | 5288 |
| SAU100546 | 4234 | 5289 |
| SAU100547 | 4235 | 5290 |
| SAU100557 | 4236 | 5291 |
| SAU100582 | 4237 | 5292 |
| SAU100590 | 4238 | 5293 |
| SAU100595 | 4239 | 5294 |
| SAU100596 | 4240 | 5295 |
| SAU100601 | 4241 | 5296 |
| SAU100608 | 4242 | 5297 |
| SAU100610 | 4243 | 5298 |
| SAU100613 | 4244 | 5299 |
| SAU100617 | 4245 | 5300 |
| SAU100633 | 4246 | 5301 |
| SAU100646 | 4247 | 5302 |
| SAU100658 | 4248 | 5303 |
| SAU100659 | 4249 | 5304 |
| SAU100679 | 4250 | 5305 |
| SAU100684 | 4251 | 5306 |
| SAU100685 | 4252 | 5307 |
| SAU100689 | 4253 | 5308 |
| SAU100690 | 4254 | 5309 |
| SAU100702 | 4255 | 5310 |
| SAU100710 | 4256 | 5311 |
| SAU100714 | 4257 | 5312 |
| SAU100731 | 4258 | 5313 |
| SAU100733 | 4259 | 5314 |
| SAU100734 | 4260 | 5315 |
| SAU100736 | 4261 | 5316 |
| SAU100738 | 4262 | 5317 |
| SAU100741 | 4263 | 5318 |
| SAU100745 | 4264 | 5319 |
| SAU100747 | 4265 | 5320 |
| SAU100751 | 4266 | 5321 |
| SAU100752 | 4267 | 5322 |
| SAU100767 | 4268 | 5323 |
| SAU100770 | 4269 | 5324 |
| SAU100771 | 4270 | 5325 |
| SAU100773 | 4271 | 5326 |
| SAU100776 | 4272 | 5327 |
| SAU100778 | 4273 | 5328 |
| SAU100793 | 4274 | 5329 |
| SAU100794 | 4275 | 5330 |
| SAU100799 | 4276 | 5331 |
| SAU100808 | 4277 | 5332 |
| SAU100810 | 4278 | 5333 |
| SAU100813 | 4279 | 5334 |
| SAU100831 | 4280 | 5335 |
| SAU100836 | 4281 | 5336 |
| SAU100838 | 4282 | 5337 |
| SAU100839 | 4283 | 5338 |
| SAU100843 | 4284 | 5339 |
| SAU100845 | 4285 | 5340 |
| SAU100858 | 4286 | 5341 |
| SAU100859 | 4287 | 5342 |
| SAU100865 | 4288 | 5343 |
| SAU100866 | 4289 | 5344 |
| SAU100879 | 4290 | 5345 |
| SAU100880 | 4291 | 5346 |
| SAU100882 | 4292 | 5347 |
| SAU100885 | 4293 | 5348 |
| SAU100886 | 4294 | 5349 |
| SAU100887 | 4295 | 5350 |
| SAU100899 | 4296 | 5351 |
| SAU100901 | 4297 | 5352 |
| SAU100916 | 4298 | 5353 |
| SAU100920 | 4299 | 5354 |
| SAU100921 | 4300 | 5355 |
| SAU100932 | 4301 | 5356 |
| SAU100944 | 4302 | 5357 |
| SAU100952 | 4303 | 5358 |
| SAU100959 | 4304 | 5359 |
| SAU100961 | 4305 | 5360 |
| SAU100962 | 4306 | 5361 |
| SAU100963 | 4307 | 5362 |
| SAU100964 | 4308 | 5363 |
| SAU100965 | 4309 | 5364 |
| SAU100970 | 4310 | 5365 |
| SAU100996 | 4311 | 5366 |
| SAU101006 | 4312 | 5367 |
| SAU101020 | 4313 | 5368 |
| SAU101024 | 4314 | 5369 |
| SAU101028 | 4315 | 5370 |
| SAU101034 | 4316 | 5371 |
| SAU101038 | 4317 | 5372 |
| SAU101039 | 4318 | 5373 |
| SAU101065 | 4319 | 5374 |
| SAU101067 | 4320 | 5375 |
| SAU101070 | 4321 | 5376 |
| SAU101084 | 4322 | 5377 |
| SAU101085 | 4323 | 5378 |
| SAU101086 | 4324 | 5379 |
| SAU101090 | 4325 | 5380 |
| SAU101092 | 4326 | 5381 |
| SAU101104 | 4327 | 5382 |
| SAU101143 | 4328 | 5383 |
| SAU101145 | 4329 | 5384 |
| SAU101155 | 4330 | 5385 |
| SAU101156 | 4331 | 5386 |
| SAU101159 | 4332 | 5387 |
| SAU101175 | 4333 | 5388 |
| SAU101180 | 4334 | 5389 |
| SAU101183 | 4335 | 5390 |
| SAU101184 | 4336 | 5391 |
| SAU101189 | 4337 | 5392 |
| SAU101197 | 4338 | 5393 |
| SAU101198 | 4339 | 5394 |
| SAU101199 | 4340 | 5395 |
| SAU101220 | 4341 | 5396 |
| SAU101224 | 4342 | 5397 |
| SAU101226 | 4343 | 5398 |
| SAU101231 | 4344 | 5399 |
| SAU101235 | 4345 | 5400 |
| SAU101236 | 4346 | 5401 |
| SAU101239 | 4347 | 5402 |
| SAU101240 | 4348 | 5403 |
| SAU101242 | 4349 | 5404 |
| SAU101247 | 4350 | 5405 |
| SAU101262 | 4351 | 5406 |
| SAU101265 | 4352 | 5407 |
| SAU101266 | 4353 | 5408 |
| SAU101267 | 4354 | 5409 |
| SAU101270 | 4355 | 5410 |
| SAU101271 | 4356 | 5411 |
| SAU101275 | 4357 | 5412 |
| SAU101286 | 4358 | 5413 |
| SAU101293 | 4359 | 5414 |
| SAU101300 | 4360 | 5415 |
| SAU101301 | 4361 | 5416 |
| SAU101302 | 4362 | 5417 |
| SAU101310 | 4363 | 5418 |
| SAU101311 | 4364 | 5419 |
| SAU101320 | 4365 | 5420 |
| SAU101327 | 4366 | 5421 |
| SAU101339 | 4367 | 5422 |
| SAU101340 | 4368 | 5423 |
| SAU101341 | 4369 | 5424 |
| SAU101343 | 4370 | 5425 |
| SAU101344 | 4371 | 5426 |
| SAU101346 | 4372 | 5427 |
| SAU101347 | 4373 | 5428 |
| SAU101350 | 4374 | 5429 |
| SAU101351 | 4375 | 5430 |
| SAU101360 | 4376 | 5431 |
| SAU101365 | 4377 | 5432 |
| SAU101366 | 4378 | 5433 |
| SAU101369 | 4379 | 5434 |
| SAU101371 | 4380 | 5435 |
| SAU101381 | 4381 | 5436 |
| SAU101382 | 4382 | 5437 |
| SAU101383 | 4383 | 5438 |
| SAU101385 | 4384 | 5439 |
| SAU101387 | 4385 | 5440 |
| SAU101389 | 4386 | 5441 |
| SAU101398 | 4387 | 5442 |
| SAU101399 | 4388 | 5443 |
| SAU101400 | 4389 | 5444 |
| SAU101408 | 4390 | 5445 |
| SAU101421 | 4391 | 5446 |
| SAU101427 | 4392 | 5447 |
| SAU101432 | 4393 | 5448 |
| SAU101436 | 4394 | 5449 |
| SAU101438 | 4395 | 5450 |
| SAU101444 | 4396 | 5451 |
| SAU101445 | 4397 | 5452 |
| SAU101446 | 4398 | 5453 |
| SAU101447 | 4399 | 5454 |
| SAU101452 | 4400 | 5455 |
| SAU101455 | 4401 | 5456 |
| SAU101461 | 4402 | 5457 |
| SAU101463 | 4403 | 5458 |
| SAU101476 | 4404 | 5459 |
| SAU101481 | 4405 | 5460 |
| SAU101482 | 4406 | 5461 |
| SAU101483 | 4407 | 5462 |
| SAU101488 | 4408 | 5463 |
| SAU101491 | 4409 | 5464 |
| SAU101492 | 4410 | 5465 |
| SAU101493 | 4411 | 5466 |
| SAU101495 | 4412 | 5467 |
| SAU101497 | 4413 | 5468 |
| SAU101509 | 4414 | 5469 |
| SAU101526 | 4415 | 5470 |
| SAU101529 | 4416 | 5471 |
| SAU101541 | 4417 | 5472 |
| SAU101543 | 4418 | 5473 |
| SAU101545 | 4419 | 5474 |
| SAU101546 | 4420 | 5475 |
| SAU101549 | 4421 | 5476 |
| SAU101551 | 4422 | 5477 |
| SAU101554 | 4423 | 5478 |
| SAU101561 | 4424 | 5479 |
| SAU101565 | 4425 | 5480 |
| SAU101567 | 4426 | 5481 |
| SAU101570 | 4427 | 5482 |
| SAU101571 | 4428 | 5483 |
| SAU101572 | 4429 | 5484 |
| SAU101573 | 4430 | 5485 |
| SAU101574 | 4431 | 5486 |
| SAU101575 | 4432 | 5487 |
| SAU101576 | 4433 | 5488 |
| SAU101586 | 4434 | 5489 |
| SAU101592 | 4435 | 5490 |
| SAU101599 | 4436 | 5491 |
| SAU101610 | 4437 | 5492 |
| SAU101612 | 4438 | 5493 |
| SAU101614 | 4439 | 5494 |
| SAU101616 | 4440 | 5495 |
| SAU101622 | 4441 | 5496 |
| SAU101624 | 4442 | 5497 |
| SAU101630 | 4443 | 5498 |
| SAU101632 | 4444 | 5499 |
| SAU101637 | 4445 | 5500 |
| SAU101641 | 4446 | 5501 |
| SAU101651 | 4447 | 5502 |
| SAU101652 | 4448 | 5503 |
| SAU101653 | 4449 | 5504 |
| SAU101655 | 4450 | 5505 |
| SAU101663 | 4451 | 5506 |
| SAU101664 | 4452 | 5507 |
| SAU101674 | 4453 | 5508 |
| SAU101679 | 4454 | 5509 |
| SAU101681 | 4455 | 5510 |
| SAU101682 | 4456 | 5511 |
| SAU101685 | 4457 | 5512 |
| SAU101717 | 4458 | 5513 |
| SAU101724 | 4459 | 5514 |
| SAU101726 | 4460 | 5515 |
| SAU101727 | 4461 | 5516 |
| SAU101728 | 4462 | 5517 |
| SAU101736 | 4463 | 5518 |
| SAU101737 | 4464 | 5519 |
| SAU101744 | 4465 | 5520 |
| SAU101751 | 4466 | 5521 |
| SAU101752 | 4467 | 5522 |
| SAU101754 | 4468 | 5523 |
| SAU101756 | 4469 | 5524 |
| SAU101771 | 4470 | 5525 |
| SAU101772 | 4471 | 5526 |
| SAU101777 | 4472 | 5527 |
| SAU101781 | 4473 | 5528 |
| SAU101782 | 4474 | 5529 |
| SAU101784 | 4475 | 5530 |
| SAU101790 | 4476 | 5531 |
| SAU101791 | 4477 | 5532 |
| SAU101792 | 4478 | 5533 |
| SAU101793 | 4479 | 5534 |
| SAU101794 | 4480 | 5535 |
| SAU101795 | 4481 | 5536 |
| SAU101797 | 4482 | 5537 |
| SAU101798 | 4483 | 5538 |
| SAU101799 | 4484 | 5539 |
| SAU101800 | 4485 | 5540 |
| SAU101801 | 4486 | 5541 |
| SAU101802 | 4487 | 5542 |
| SAU101803 | 4488 | 5543 |
| SAU101804 | 4489 | 5544 |
| SAU101805 | 4490 | 5545 |
| SAU101806 | 4491 | 5546 |
| SAU101807 | 4492 | 5547 |
| SAU101808 | 4493 | 5548 |
| SAU101810 | 4494 | 5549 |
| SAU101811 | 4495 | 5550 |
| SAU101814 | 4496 | 5551 |
| SAU101815 | 4497 | 5552 |
| SAU101818 | 4498 | 5553 |
| SAU101824 | 4499 | 5554 |
| SAU101833 | 4500 | 5555 |
| SAU101839 | 4501 | 5556 |
| SAU101842 | 4502 | 5557 |
| SAU101845 | 4503 | 5558 |
| SAU101849 | 4504 | 5559 |
| SAU101857 | 4505 | 5560 |
| SAU101862 | 4506 | 5561 |
| SAU101864 | 4507 | 5562 |
| SAU101865 | 4508 | 5563 |
| SAU101866 | 4509 | 5564 |
| SAU101868 | 4510 | 5565 |
| SAU101869 | 4511 | 5566 |
| SAU101876 | 4512 | 5567 |
| SAU101881 | 4513 | 5568 |
| SAU101882 | 4514 | 5569 |
| SAU101890 | 4515 | 5570 |
| SAU101891 | 4516 | 5571 |
| SAU101893 | 4517 | 5572 |
| SAU101904 | 4518 | 5573 |
| SAU101907 | 4519 | 5574 |
| SAU101909 | 4520 | 5575 |
| SAU101910 | 4521 | 5576 |
| SAU101915 | 4522 | 5577 |
| SAU101922 | 4523 | 5578 |
| SAU101948 | 4524 | 5579 |
| SAU101966 | 4525 | 5580 |
| SAU101968 | 4526 | 5581 |
| SAU101991 | 4527 | 5582 |
| SAU101995 | 4528 | 5583 |
| SAU101996 | 4529 | 5584 |
| SAU101999 | 4530 | 5585 |
| SAU102001 | 4531 | 5586 |
| SAU102002 | 4532 | 5587 |
| SAU102003 | 4533 | 5588 |
| SAU102006 | 4534 | 5589 |
| SAU102007 | 4535 | 5590 |
| SAU102032 | 4536 | 5591 |
| SAU102035 | 4537 | 5592 |
| SAU102044 | 4538 | 5593 |
| SAU102046 | 4539 | 5594 |
| SAU102049 | 4540 | 5595 |
| SAU102054 | 4541 | 5596 |
| SAU102059 | 4542 | 5597 |
| SAU102067 | 4543 | 5598 |
| SAU102068 | 4544 | 5599 |
| SAU102102 | 4545 | 5600 |
| SAU102113 | 4546 | 5601 |
| SAU102116 | 4547 | 5602 |
| SAU102117 | 4548 | 5603 |
| SAU102129 | 4549 | 5604 |
| SAU102132 | 4550 | 5605 |
| SAU102142 | 4551 | 5606 |
| SAU102143 | 4552 | 5607 |
| SAU102144 | 4553 | 5608 |
| SAU102162 | 4554 | 5609 |
| SAU102165 | 4555 | 5610 |
| SAU102200 | 4556 | 5611 |
| SAU102201 | 4557 | 5612 |
| SAU102222 | 4558 | 5613 |
| SAU102231 | 4559 | 5614 |
| SAU102232 | 4560 | 5615 |
| SAU102233 | 4561 | 5616 |
| SAU102241 | 4562 | 5617 |
| SAU102242 | 4563 | 5618 |
| SAU102246 | 4564 | 5619 |
| SAU102247 | 4565 | 5620 |
| SAU102252 | 4566 | 5621 |
| SAU102256 | 4567 | 5622 |
| SAU102257 | 4568 | 5623 |
| SAU102259 | 4569 | 5624 |
| SAU102260 | 4570 | 5625 |
| SAU102261 | 4571 | 5626 |
| SAU102262 | 4572 | 5627 |
| SAU102264 | 4573 | 5628 |
| SAU102265 | 4574 | 5629 |
| SAU102268 | 4575 | 5630 |
| SAU102270 | 4576 | 5631 |
| SAU102280 | 4577 | 5632 |
| SAU102281 | 4578 | 5633 |
| SAU102283 | 4579 | 5634 |
| SAU102284 | 4580 | 5635 |
| SAU102286 | 4581 | 5636 |
| SAU102287 | 4582 | 5637 |
| SAU102292 | 4583 | 5638 |
| SAU102294 | 4584 | 5639 |
| SAU102297 | 4585 | 5640 |
| SAU102298 | 4586 | 5641 |
| SAU102308 | 4587 | 5642 |
| SAU102318 | 4588 | 5643 |
| SAU102333 | 4589 | 5644 |
| SAU102334 | 4590 | 5645 |
| SAU102336 | 4591 | 5646 |
| SAU102340 | 4592 | 5647 |
| SAU102345 | 4593 | 5648 |
| SAU102350 | 4594 | 5649 |
| SAU102352 | 4595 | 5650 |
| SAU102355 | 4596 | 5651 |
| SAU102356 | 4597 | 5652 |
| SAU102378 | 4598 | 5653 |
| SAU102380 | 4599 | 5654 |
| SAU102388 | 4600 | 5655 |
| SAU102389 | 4601 | 5656 |
| SAU102390 | 4602 | 5657 |
| SAU102392 | 4603 | 5658 |
| SAU102394 | 4604 | 5659 |
| SAU102396 | 4605 | 5660 |
| SAU102401 | 4606 | 5661 |
| SAU102407 | 4607 | 5662 |
| SAU102417 | 4608 | 5663 |
| SAU102418 | 4609 | 5664 |
| SAU102420 | 4610 | 5665 |
| SAU102422 | 4611 | 5666 |
| SAU102423 | 4612 | 5667 |
| SAU102433 | 4613 | 5668 |
| SAU102434 | 4614 | 5669 |
| SAU102437 | 4615 | 5670 |
| SAU102440 | 4616 | 5671 |
| SAU102447 | 4617 | 5672 |
| SAU102448 | 4618 | 5673 |
| SAU102449 | 4619 | 5674 |
| SAU102450 | 4620 | 5675 |
| SAU102452 | 4621 | 5676 |
| SAU102453 | 4622 | 5677 |
| SAU102460 | 4623 | 5678 |
| SAU102469 | 4624 | 5679 |
| SAU102473 | 4625 | 5680 |
| SAU102474 | 4626 | 5681 |
| SAU102476 | 4627 | 5682 |
| SAU102479 | 4628 | 5683 |
| SAU102480 | 4629 | 5684 |
| SAU102481 | 4630 | 5685 |
| SAU102485 | 4631 | 5686 |
| SAU102486 | 4632 | 5687 |
| SAU102487 | 4633 | 5688 |
| SAU102498 | 4634 | 5689 |
| SAU102502 | 4635 | 5690 |
| SAU102503 | 4636 | 5691 |
| SAU102526 | 4637 | 5692 |
| SAU102527 | 4638 | 5693 |
| SAU102531 | 4639 | 5694 |
| SAU102533 | 4640 | 5695 |
| SAU102534 | 4641 | 5696 |
| SAU102541 | 4642 | 5697 |
| SAU102551 | 4643 | 5698 |
| SAU102554 | 4644 | 5699 |
| SAU102575 | 4645 | 5700 |
| SAU102578 | 4646 | 5701 |
| SAU102584 | 4647 | 5702 |
| SAU102585 | 4648 | 5703 |
| SAU102593 | 4649 | 5704 |
| SAU102598 | 4650 | 5705 |
| SAU102599 | 4651 | 5706 |
| SAU102601 | 4652 | 5707 |
| SAU102602 | 4653 | 5708 |
| SAU102603 | 4654 | 5709 |
| SAU102605 | 4655 | 5710 |
| SAU102606 | 4656 | 5711 |
| SAU102607 | 4657 | 5712 |
| SAU102609 | 4658 | 5713 |
| SAU102610 | 4659 | 5714 |
| SAU102613 | 4660 | 5715 |
| SAU102614 | 4661 | 5716 |
| SAU102615 | 4662 | 5717 |
| SAU102620 | 4663 | 5718 |
| SAU102621 | 4664 | 5719 |
| SAU102629 | 4665 | 5720 |
| SAU102631 | 4666 | 5721 |
| SAU102636 | 4667 | 5722 |
| SAU102637 | 4668 | 5723 |
| SAU102639 | 4669 | 5724 |
| SAU102652 | 4670 | 5725 |
| SAU102658 | 4671 | 5726 |
| SAU102663 | 4672 | 5727 |
| SAU102669 | 4673 | 5728 |
| SAU102671 | 4674 | 5729 |
| SAU102674 | 4675 | 5730 |
| SAU102693 | 4676 | 5731 |
| SAU102694 | 4677 | 5732 |
| SAU102725 | 4678 | 5733 |
| SAU102764 | 4679 | 5734 |
| SAU102766 | 4680 | 5735 |
| SAU102812 | 4681 | 5736 |
| SAU102863 | 4682 | 5737 |
| SAU102870 | 4683 | 5738 |
| SAU102880 | 4684 | 5739 |
| SAU102881 | 4685 | 5740 |
| SAU102883 | 4686 | 5741 |
| SAU102905 | 4687 | 5742 |
| SAU102909 | 4688 | 5743 |
| SAU102933 | 4689 | 5744 |
| SAU102935 | 4690 | 5745 |
| SAU102936 | 4691 | 5746 |
| SAU102939 | 4692 | 5747 |
| SAU102942 | 4693 | 5748 |
| SAU102944 | 4694 | 5749 |
| SAU102979 | 4695 | 5750 |
| SAU102983 | 4696 | 5751 |
| SAU102992 | 4697 | 5752 |
| SAU103010 | 4698 | 5753 |
| SAU103024 | 4699 | 5754 |
| SAU103025 | 4700 | 5755 |
| SAU103037 | 4701 | 5756 |
| SAU103038 | 4702 | 5757 |
| SAU103042 | 4703 | 5758 |
| SAU103077 | 4704 | 5759 |
| SAU103115 | 4705 | 5760 |
| SAU103144 | 4706 | 5761 |
| SAU103159 | 4707 | 5762 |
| SAU103169 | 4708 | 5763 |
| SAU103175 | 4709 | 5764 |
| SAU103191 | 4710 | 5765 |
| SAU103198 | 4711 | 5766 |
| SAU103204 | 4712 | 5767 |
| SAU103226 | 4713 | 5768 |
| SAU103232 | 4714 | 5769 |
| SAU200006 | 4715 | 5770 |
| SAU200028 | 4716 | 5771 |
| SAU200030 | 4717 | 5772 |
| SAU200058 | 4718 | 5773 |
| SAU200059 | 4719 | 5774 |
| SAU200088 | 4720 | 5775 |
| SAU200157 | 4721 | 5776 |
| SAU200242 | 4722 | 5777 |
| SAU200297 | 4723 | 5778 |
| SAU200345 | 4724 | 5779 |
| SAU200392 | 4725 | 5780 |
| SAU200468 | 4726 | 5781 |
| SAU200558 | 4727 | 5782 |
| SAU200561 | 4728 | 5783 |
| SAU200564 | 4729 | 5784 |
| SAU200565 | 4730 | 5785 |
| SAU200593 | 4731 | 5786 |
| SAU200601 | 4732 | 5787 |
| SAU200628 | 4733 | 5788 |
| SAU200657 | 4734 | 5789 |
| SAU200685 | 4735 | 5790 |
| SAU200721 | 4736 | 5791 |
| SAU200725 | 4737 | 5792 |
| SAU200731 | 4738 | 5793 |
| SAU200740 | 4739 | 5794 |
| SAU200752 | 4740 | 5795 |
| SAU200914 | 4741 | 5796 |
| SAU200916 | 4742 | 5797 |
| SAU200928 | 4743 | 5798 |
| SAU200934 | 4744 | 5799 |
| SAU200949 | 4745 | 5800 |
| SAU200960 | 4746 | 5801 |
| SAU200994 | 4747 | 5802 |
| SAU201167 | 4748 | 5803 |
| SAU201168 | 4749 | 5804 |
| SAU201184 | 4750 | 5805 |
| SAU201197 | 4751 | 5806 |
| SAU201225 | 4752 | 5807 |
| SAU201236 | 4753 | 5808 |
| SAU201301 | 4754 | 5809 |
| SAU201333 | 4755 | 5810 |
| SAU201375 | 4756 | 5811 |
| SAU201380 | 4757 | 5812 |
| SAU201381 | 4758 | 5813 |
| SAU201385 | 4759 | 5814 |
| SAU201403 | 4760 | 5815 |
| SAU201469 | 4761 | 5816 |
| SAU201486 | 4762 | 5817 |
| SAU201506 | 4763 | 5818 |
| SAU201508 | 4764 | 5819 |
| SAU201513 | 4765 | 5820 |
| SAU201539 | 4766 | 5821 |
| SAU201541 | 4767 | 5822 |
| SAU201558 | 4768 | 5823 |
| SAU201571 | 4769 | 5824 |
| SAU201611 | 4770 | 5825 |
| SAU201615 | 4771 | 5826 |
| SAU201620 | 4772 | 5827 |
| SAU201621 | 4773 | 5828 |
| SAU201654 | 4774 | 5829 |
| SAU201666 | 4775 | 5830 |
| SAU201743 | 4776 | 5831 |
| SAU201752 | 4777 | 5832 |
| SAU201765 | 4778 | 5833 |
| SAU201773 | 4779 | 5834 |
| SAU201775 | 4780 | 5835 |
| SAU201810 | 4781 | 5836 |
| SAU201827 | 4782 | 5837 |
| SAU201929 | 4783 | 5838 |
| SAU201952 | 4784 | 5839 |
| SAU201961 | 4785 | 5840 |
| SAU201971 | 4786 | 5841 |
| SAU202006 | 4787 | 5842 |
| SAU202039 | 4788 | 5843 |
| SAU202126 | 4789 | 5844 |
| SAU202174 | 4790 | 5845 |
| SAU202176 | 4791 | 5846 |
| SAU202186 | 4792 | 5847 |
| SAU202267 | 4793 | 5848 |
| SAU202708 | 4794 | 5849 |
| SAU202731 | 4795 | 5850 |
| SAU202736 | 4796 | 5851 |
| SAU202756 | 4797 | 5852 |
| SAU202781 | 4798 | 5853 |
| SAU202872 | 4799 | 5854 |
| SAU202882 | 4800 | 5855 |
| SAU202930 | 4801 | 5856 |
| SAU202945 | 4802 | 5857 |
| SAU202968 | 4803 | 5858 |
| SAU203001 | 4804 | 5859 |
| SAU203007 | 4805 | 5860 |
| SAU203196 | 4806 | 5861 |
| SAU203293 | 4807 | 5862 |
| SAU203296 | 4808 | 5863 |
| SAU203524 | 4809 | 5864 |
| SAU300110 | 4810 | 5865 |
| SAU300131 | 4811 | 5866 |
| SAU300156 | 4812 | 5867 |
| SAU300191 | 4813 | 5868 |
| SAU300269 | 4814 | 5869 |
| SAU300335 | 4815 | 5870 |
| SAU300338 | 4816 | 5871 |
| SAU300455 | 4817 | 5872 |
| SAU300572 | 4818 | 5873 |
| SAU300617 | 4819 | 5874 |
| SAU300713 | 4820 | 5875 |
| SAU300719 | 4821 | 5876 |
| SAU300732 | 4822 | 5877 |
| SAU300825 | 4823 | 5878 |
| SAU300868 | 4824 | 5879 |
| SAU300975 | 4825 | 5880 |
| SAU300998 | 4826 | 5881 |
| SAU301004 | 4827 | 5882 |
| SAU301030 | 4828 | 5883 |
| SAU301054 | 4829 | 5884 |
| SAU301080 | 4830 | 5885 |
| SAU301118 | 4831 | 5886 |
| SAU301133 | 4832 | 5887 |
| SAU301148 | 4833 | 5888 |
| SAU301223 | 4834 | 5889 |
| SAU301230 | 4835 | 5890 |
| SAU301268 | 4836 | 5891 |
| SAU301275 | 4837 | 5892 |
| SAU301357 | 4838 | 5893 |
| SAU301363 | 4839 | 5894 |
| SAU301433 | 4840 | 5895 |
| SAU301465 | 4841 | 5896 |
| SAU301472 | 4842 | 5897 |
| SAU301592 | 4843 | 5898 |
| SAU301620 | 4844 | 5899 |
| SAU301758 | 4845 | 5900 |
| SAU301773 | 4846 | 5901 |
| SAU301829 | 4847 | 5902 |
| SAU301869 | 4848 | 5903 |
| SAU301898 | 4849 | 5904 |
| SAU302060 | 4850 | 5905 |
| SAU302513 | 4851 | 5906 |
| SAU302626 | 4852 | 5907 |
| SAU302685 | 4853 | 5908 |
| SAU302698 | 4854 | 5909 |
| SAU302699 | 4855 | 5910 |
| SAU302805 | 4856 | 5911 |
| SAU302901 | 4857 | 5912 |
| SAU302931 | 4858 | 5913 |
| SAU302950 | 4859 | 5914 |
| SAU302956 | 4860 | 5915 |

What is claimed is:

1. A purified or isolated nucleic acid sequence comprising a nucleotide sequence consisting essentially of one of SEQ ID NOs: 8-3795, wherein expression of said nucleic acid inhibits proliferation of a cell.

2. A purified or isolated nucleic acid comprising a fragment of one of SEQ ID NOs.: 8-3795, said fragment selected from the group consisting of fragments comprising at least 10, at least 20, at least 25, at least 30, at least 50 and more than 50 consecutive nucleotides of one of SEQ ID NOs: 8-3795.

3. A purified or isolated antisense nucleic acid comprising a nucleotide sequence complementary to at least a portion of an intragenic sequence, intergenic sequence, sequences spanning at least a portion of two or more genes, 5′ noncoding region, or 3′ noncoding region within an operon comprising a proliferation-required gene whose activity or expression is inhibited by an antisense nucleic acid comprising the nucleotide sequence of one of SEQ ID NOs.: 8-3795.

4. A purified or isolated nucleic acid comprising a nucleotide sequence having at least 70% identity to a nucleotide sequence selected from the group consisting of SEQ ID NOs.: 8-3795, fragments comprising at least 25 consecutive nucleotides of SEQ ID NOs.: 8-3795, the nucleotide sequences complementary to SEQ ID NOs.: 8-3795 and the sequences complementary to fragments comprising at least 25 consecutive nucleotides of SEQ ID NOs.: 8-3795 as determined using BLASTN version 2.0 with the default parameters.

5. A vector comprising a promoter operably linked to a nucleic acid encoding a polypeptide whose expression is inhibited by an antisense nucleic acid comprising a nucleotide sequence of any one of SEQ ID NOs.: 8-3795.

6. A purified or isolated polypeptide comprising a polypeptide whose expression is inhibited by an antisense nucleic acid comprising a nucleotide sequence of any one of SEQ ID NOs.: 8-3795, or a fragment selected from the group consisting of fragments comprising at least 5, at least 10, at least 20, at least 30, at least 40, at least 50, at least 60 or more than 60 consecutive amino acids of one of the said polypeptides.

7. A purified or isolated polypeptide comprising a polypeptide having at least 25% amino acid identity to a polypeptide whose expression is inhibited by a nucleic acid comprising a nucleotide sequence selected from the group consisting of SEQ ID NOs.: 8-3795, or at least 25% amino acid identity to a fragment comprising at least 10, at least 20, at least 30, at least 40, at least 50, at least 60 or more than 60 consecutive amino acids of a polypeptide whose expression is inhibited by a nucleic acid comprising a nucleotide sequence selected from the group consisting of SEQ ID NOs.: 8-3795 as determined using FASTA version 3.0t78 with the default parameters.

8. A method of producing a polypeptide, comprising introducing a vector comprising a promoter operably linked to a nucleic acid comprising a nucleotide sequence encoding a polypeptide whose expression is inhibited by an antisense nucleic acid comprising one of SEQ ID NOs.: 8-3795 into a cell.

9. A method of inhibiting proliferation of a cell in an individual comprising inhibiting the activity or reducing the amount of a gene product whose expression is inhibited by an antisense nucleic acid comprising a nucleotide sequence selected from the group consisting of SEQ ID NOs.: 8-3795 or inhibiting the activity or reducing the amount of a nucleic acid encoding said gene product.

10. A method for identifying a compound which influences the activity of a gene product required for proliferation, said gene product comprising a gene product whose expression is inhibited by an antisense nucleic acid comprising a nucleotide sequence selected from the group consisting of SEQ ID NOs.: 8-3795, said method comprising:
contacting said gene product with a candidate compound; and
determining whether said compound influences the activity of said gene product.

11. A method for identifying a compound or nucleic acid having the ability to reduce the activity or level of a gene product required for proliferation, said gene product comprising a gene product whose activity or expression is inhibited by an antisense nucleic acid comprising a nucleotide sequence selected from the group consisting of SEQ ID NOs.: 8-3795, said method comprising:
(a) contacting a target gene or RNA encoding said gene product with a candidate compound or nucleic acid; and
(b) measuring an activity of said target.

12. A method for identifying a compound which reduces the activity or level of a gene product required for proliferation of a cell, wherein the activity or expression of said gene product is inhibited by an antisense nucleic acid comprising a nucleotide sequence selected from the group consisting of SEQ ID NOs.: 8-3795, said method comprising the steps of:
(a) providing a sublethal level of an antisense nucleic acid comprising a nucleotide sequence complementary to a nucleic acid comprising a nucleotide sequence encoding said gene product in a cell to reduce the activity or amount of said gene product in said cell, thereby producing a sensitized cell;
(b) contacting said sensitized cell with a compound; and
(c) determining the degree to which said compound inhibits proliferation of said sensitized cell relative to a cell which does not contain said antisense nucleic acid.

13. A method for inhibiting cellular proliferation comprising introducing an effective amount of a compound with activity against a gene whose activity or expression is inhibited by an antisense nucleic acid comprising a nucleotide sequence selected from the group consisting of SEQ ID NOs.: 8-3795 or a compound with activity against the product of said gene into a population of cells expressing said gene.

14. A composition comprising an effective concentration of an antisense nucleic acid comprising a nucleotide sequence selected from the group consisting of SEQ ID NOs.: 8-3795, or a proliferation-inhibiting portion thereof in a pharmaceutically acceptable carrier.

15. A method for inhibiting the activity or expression of a gene in an operon required for proliferation wherein the activity or expression of at least one gene in said operon is inhibited by an antisense nucleic acid comprising a sequence selected from the group consisting of SEQ ID NOs.: 8-3795, said method comprising contacting a cell in a cell population with an antisense nucleic acid complementary to at least a portion of said operon.

16. A method for identifying a gene which is required for proliferation of a cell comprising:
(a) contacting a cell with an antisense nucleic acid comprising a nucleotide sequence selected from the group consisting of SEQ ID NOs.: 8-3795, wherein said cell is a cell other than the organism from which said nucleic acid was obtained;
(b) determining whether said nucleic acid inhibits proliferation of said cell; and
(c) identifying the gene in said cell which encodes the mRNA which is complementary to said antisense nucleic acid or a portion thereof.

17. A method for identifying a compound having the ability to inhibit proliferation of a cell comprising:
(a) identifying a homolog of a gene or gene product whose activity or level is inhibited by a nucleic acid comprising a nucleotide sequence selected from the group consisting of SEQ ID NOs. 8-3795 in a test cell, wherein said test cell is not the cell from which said nucleic acid was obtained;
(b) identifying an inhibitory nucleic acid sequence which inhibits the activity of said homolog in said test cell;
(c) contacting said test cell with a sublethal level of said inhibitory nucleic acid, thus sensitizing said cell;
(d) contacting the sensitized cell of step (c) with a compound; and
(e) determining the degree to which said compound inhibits proliferation of said sensitized cell relative to a cell which does not contain said inhibitory nucleic acid.

18. A method of identifying a compound having the ability to inhibit proliferation comprising:
(a) contacting a test cell with a sublethal level of a nucleic acid comprising a nucleotide sequence selected from the group consisting of SEQ ID NOs. 8-3795 or a portion thereof which inhibits the proliferation of the cell from which said nucleic acid was obtained, thus sensitizing said test cell;
(b) contacting the sensitized test cell of step (a) with a compound; and
(c) determining the degree to which said compound inhibits proliferation of said sensitized test cell relative to a cell which does not contain said nucleic acid.

19. A method for identifying a compound having activity against a biological pathway required for proliferation comprising:
(a) sensitizing a cell by providing a sublethal level of an antisense nucleic acid complementary to a nucleic acid encoding a gene product required for proliferation, wherein the activity or expression of said gene product is inhibited by an antisense nucleic acid comprising a nucleotide sequence selected from the group consisting of SEQ ID NOs.: 8-3795, in said cell to reduce the activity or amount of said gene product;
(b) contacting the sensitized cell with a compound; and
(c) determining the degree to which said compound inhibits the growth of said sensitized cell relative to a cell which does not contain said antisense nucleic acid.

20. A method for identifying a compound having the ability to inhibit cellular proliferation comprising:
(a) contacting a cell with an agent which reduces the activity or level of a gene product required for proliferation of said cell, wherein said gene product is a gene product whose activity or expression is inhibited by an antisense nucleic acid comprising a nucleotide sequence selected from the group consisting of SEQ ID NOs.: 8-3795;
(b) contacting said cell with a compound; and
(c) determining whether said compound reduces proliferation of said contacted cell by acting on said gene product.

21. A method for identifying the biological pathway in which a proliferation-required gene or its gene product lies, wherein said gene or gene product comprises a gene or gene product whose activity or expression is inhibited by an antisense nucleic acid comprising a sequence selected from the group consisting of SEQ ID NOs.: 8-3795, said method comprising:
(a) providing a sublethal level of an antisense nucleic acid which inhibits the activity of said proliferation-required gene or gene product in a test cell;
(b) contacting said test cell with a compound known to inhibit growth or proliferation of a cell, wherein the biological pathway on which said compound acts is known; and
(c) determining the degree to which said proliferation of said test cell is inhibited relative to a cell which was not contacted with said compound.

22. A method for determining the biological pathway on which a test compound acts comprising:
(a) providing a sublethal level of an antisense nucleic acid complementary to a proliferation-required nucleic acid in a first cell, wherein the activity or expression of said proliferation-required nucleic acid is inhibited by an antisense nucleic acid comprising a sequence selected from the group consisting of SEQ ID NOs.: 8-3795 and wherein the biological pathway in which said proliferation-required nucleic acid or a protein encoded by said proliferation-required nucleic acid lies is known,
(b) contacting said first cell with said test compound; and
(c) determining the degree to which said test compound inhibits proliferation of said first cell relative to a cell which does not contain said antisense nucleic acid.

23. A purified or isolated nucleic acid comprising a sequence selected from the group consisting of SEQ ID NOs.: 8-3795.

24. A compound which interacts with a gene or gene product whose activity or expression is inhibited by an antisense nucleic acid comprising a nucleotide sequence of one of SEQ ID NOs.: 8-3795 to inhibit proliferation.

25. A compound which interacts with a gene product whose expression is inhibited by an antisense nucleic acid comprising a nucleotide sequence of one of SEQ ID NOs.: 8-3795 to inhibit proliferation.

26. A method for manufacturing an antibiotic comprising the steps of:
screening one or more candidate compounds to identify a compound that reduces the activity or level of a gene product required for proliferation, said gene product comprising a gene product whose activity or expression is inhibited by an antisense nucleic acid comprising a nucleotide sequence selected from the group consisting of SEQ ID NOs.: 8-3795; and
manufacturing the compound so identified.

27. A purified or isolated nucleic acid comprising a nucleic acid having at least 70% nucleotide sequence identity to a nucleotide sequence selected from the group consisting of SEQ ID NOs.: 3796-3800, 3806-4860, 5916-10012, fragments comprising at least 25 consecutive nucleotides of SEQ ID NOs.: 3796-3800, 3806-4860, 5916-10012, the nucleotide sequences complementary to SEQ ID NOs.:3796-3800, 3806-4860, 5916-10012, and the nucleotide sequences complementary to fragments comprising at least 25 consecutive nucleotides of SEQ ID NOs.: 3796-3800, 3806-4860, 5916-10012 as determined using BLASTN version 2.0 with the default parameters.

28. A method of inhibiting proliferation of a cell comprising inhibiting the activity or reducing the amount of a gene product in said cell or inhibiting the activity or reducing the amount of a nucleic acid encoding said gene product in said cell, wherein said gene product is selected from the group consisting of a gene product having having at least 70% nucleotide sequence identity as determined using BLASTN version 2.0 with the default parameters to a gene product whose expression is inhibited by an antisense nucleic acid comprising a nucleotide sequence selected from the group consisting of SEQ ID NOs.: 8-3795, a gene product encoded by a nucleic acid having at least 70% nucleotide sequence identity as determined using BLASTN version 2.0 with the default parameters to a nucleic acid encoding a gene product whose expression is inhibited by an antisense nucleic acid comprising a nucleotide sequence selected from the group consisting of SEQ ID NOs:8-3795, a gene product having at least 25% amino acid identity as determined using FASTA version 3.0t78 with the default parameters to a gene product whose expression is inhibited by an antisense nucleic acid comprising a nucleotide sequence selected from the group consisting of SEQ ID NOs.: 8-3795, a gene product encoded by a nucleic acid which hybridizes to a nucleic acid comprising a nucleotide sequence selected from the group consisting of SEQ ID NOs.: 8-3795 under stringent conditions, a gene product encoded by a nucleic acid which hybridizes to a nucleic acid comprising a nucleotide sequence selected from the group consisting of SEQ ID NOs.: 8-3795 under moderate conditions, and a gene product whose activity may be complemented by the gene product whose activity is inhibited by a nucleic acid comprising a nucleotide sequence selected from the group consisting of SEQ ID NOs: 8-3795.

29. A method for identifying a compound which influences the activity of a gene product required for proliferation comprising:
contacting a candidate compound with a gene product selected from the group consisting of a gene product having at least 70% nucleotide sequence identity as determined using BLASTN version 2.0 with the default parameters to a gene product whose expression is inhibited by an antisense nucleic acid comprising a nucleotide sequence selected from the group consisting of SEQ ID NOs.: 8-3795, a gene product encoded by a nucleic acid having at least 70% nucleotide sequence identity as determined using BLASTN version 2.0 with the default parameters to a nucleic acid encoding a gene product whose expression is inhibited by an antisense nucleic acid comprising a nucleotide sequence selected from the group consisting of SEQ ID NOs:8-3795, a gene product having at least 25% amino acid identity as determined using FASTA version 3.0t78 with the default parameters to a gene product whose expression is inhibited by an antisense nucleic acid comprising a nucleotide sequence selected from the group consisting of SEQ ID NOs.: 8-3795, a gene product encoded by a nucleic acid comprising a nucleotide sequence which hybridizes to a nucleic acid selected from the group consisting of SEQ ID NOs.: 8-3795 under stringent conditions, a gene product encoded by a nucleic acid comprising a nucleotide sequence which hybridizes to a nucleic acid selected from the group consisting of SEQ ID NOs.: 8-3795 under moderate conditions, and a gene product whose activity may be complemented by the gene product whose activity is inhibited by a nucleic acid selected from the group consisting of SEQ ID NOs: 8-3795; and
determining whether said candidate compound influences the activity of said gene product.

30. A method for identifying a compound or nucleic acid having the ability to reduce the activity or level of a gene product required for proliferation comprising:
(a) providing a target that is a gene or RNA, wherein said target comprises a nucleic acid that encodes a gene product selected from the group consisting of a gene product having having at least 70% nucleotide sequence identity as determined using BLASTN version 2.0 with the default parameters to a gene product whose expression is inhibited by an antisense nucleic acid comprising a nucleotide sequence selected from the group consisting of SEQ ID NOs.: 8-3795, a gene product encoded by a nucleic acid having at least 70% nucleic acid identity as determined using BLASTN version 2.0 with the default parameters to a nucleic acid encoding a gene product whose expression is inhibited by an antisense nucleic acid comprising a nucleotide sequence selected from the group consisting of SEQ ID NOs:8-3795, a gene product having at least 25% amino acid identity as determined using FASTA version 3.0t78 with the default parameters to a gene product whose expression is inhibited by an antisense nucleic acid comprising a sequence selected from the group consisting of SEQ ID NOs.: 8-3795, a gene product encoded by a nucleic acid comprising a nucleotide sequence which hybridizes to a nucleic acid selected from the group consisting of SEQ ID NOs.: 8-3795 under stringent conditions, a gene product encoded by a nucleic acid comprising a nucleotide sequence which hybridizes to a nucleic acid selected from the group consisting of SEQ ID NOs.: 8-3795 under moderate conditions, and a gene product whose activity may be complemented by the gene product whose activity is inhibited by a nucleic acid selected from the group consisting of SEQ ID NOs: 8-3795;
(b) contacting said target with a candidate compound or nucleic acid; and
(c) measuring an activity of said target.

31. A method for identifying a compound which reduces the activity or level of a gene product required for proliferation of a cell comprising:
(a) providing a sublethal level of an antisense nucleic acid complementary to a nucleic acid encoding said gene product in a cell to reduce the activity or amount of said gene product in said cell, thereby producing a sensitized cell, wherein said gene product is selected from the group consisting of a gene product having having at least 70% nucleic acid identity as determined using BLASTN version 2.0 with the default parameters to a gene product whose expression is inhibited by an antisense nucleic acid comprising a nucleotide sequence selected from the group consisting of SEQ ID NOs.: 8-3795, a gene product encoded by a nucleic acid having at least 70% nucleotide sequence identity as determined using BLASTN version 2.0 with the default parameters to a nucleic acid encoding a gene product whose expression is inhibited by an antisense nucleic acid comprising a nucleotide sequence selected from the group consisting of SEQ ID NOs:8-3795, a gene product having at least 25% amino acid identity as determined using FASTA version 3.0t78 with the default parameters to a gene product whose expression is inhibited by an antisense nucleic acid comprising a nucleotide sequence selected from the group consisting of SEQ ID NOs.: 8-3795, a gene product encoded by a nucleic acid comprising a nucleotide sequence which hybridizes to a nucleic acid selected from the group consisting of SEQ ID NOs.: 8-3795 under stringent conditions, a gene product encoded by a nucleic acid comprising a nucleotide sequence which hybridizes to a nucleic acid comprising a nucleotide sequence selected from the group consisting of SEQ ID NOs.: 8-3795 under moderate conditions, and a gene product whose activity may be complemented by the gene product whose activity is inhibited by a nucleic acid selected from the group consisting of SEQ ID NOs: 8-3795;
(b) contacting said sensitized cell with a compound; and
(c) determining the degree to which said compound inhibits the growth of said sensitized cell relative to a cell which does not contain said antisense nucleic acid.

32. A method for inhibiting cellular proliferation comprising introducing a compound with activity against a gene product or a compound with activity against a gene encoding said gene product into a population of cells expressing said gene product, wherein said gene product is selected from the group consisting of a gene product having at least 70% nucleotide sequence identity as determined using BLASTN version 2.0 with the default parameters to a gene product whose expression is inhibited by an antisense nucleic acid comprising a nucleotide sequence selected from the group consisting of SEQ ID NOs.: 8-3795, a gene product encoded by a nucleic acid having at least 70% nucleotide sequence identity as determined using BLASTN version 2.0 with the default parameters to a nucleic acid encoding a gene product whose expression is inhibited by an antisense nucleic acid comprising a nucleotide sequence selected from the group consisting of SEQ ID NOs:8-3795, a gene product having at least 25% amino acid identity as determined using FASTA version 3.0t78 with the default parameters to a gene product whose expression is inhibited by an antisense nucleic acid comprising a nucleotide sequence selected from the group consisting of SEQ ID NOs.: 8-3795, a gene product encoded by a nucleic acid comprising a nucleotide sequence which hybridizes to a nucleic acid selected from the group consisting of SEQ ID NOs.: 8-3795 under stringent conditions, a gene product encoded by a nucleic acid comprising a nucleotide sequence which hybridizes to a nucleic acid selected from the group consisting of SEQ ID NOs.: 8-3795 under moderate conditions, and a gene product whose activity may be complemented by the gene product whose activity is inhibited by a nucleic acid selected from the group consisting of SEQ ID NOs: 8-3795.

33. A preparation comprising an effective concentration of an antisense nucleic acid in a pharmaceutically acceptable carrier wherein said antisense nucleic acid is selected from the group consisting of a nucleic acid comprising a sequence having at least 70% nucleotide sequence identity as determined using BLASTN version 2.0 with the default parameters to a nucleotide sequence selected from the group consisting of SEQ ID NOs.: 8-3795 or a proliferation-inhibiting portion thereof, a nucleic acid comprising a nucleotide sequence which hybridizes to a nucleic acid selected from the group consisting of SEQ ID NOs.: 8-3795 under stringent conditions, and a nucleic acid comprising a nucleotide sequence which hybridizes to a nucleic acid selected from the group consisting of SEQ ID NOs.: 8-3795 under moderate conditions.

34. A method for inhibiting the activity or expression of a gene in an operon which encodes a gene product required for proliferation comprising contacting a cell in a cell population with an antisense nucleic acid comprising at least a proliferation-inhibiting portion of said operon in an antisense orientation, wherein said gene product is selected from the group consisting of a gene product having at least 70% nucleotide sequence identity as determined using BLASTN version 2.0 with the default parameters to a gene product whose expression is inhibited by an antisense nucleic acid comprising a nucleotide sequence selected from the group consisting of SEQ ID NOs.: 8-3795, a gene product encoded by a nucleic acid having at least 70% nucleotide sequence identity as determined using BLASTN version 2.0 with the default parameters to a nucleic acid encoding a gene product whose expression is inhibited by an antisense nucleic acid comprising a nucleotide sequence selected from the group consisting of SEQ ID NOs:8-3795, a gene product having at least 25% amino acid identity as determined using FASTA version 3.0t78 with the default parameters to a gene product whose expression is inhibited by an antisense nucleic acid comprising a nucleotide sequence selected from the group consisting of SEQ ID NOs.: 8-3795, a gene product encoded by a nucleic acid comprising a nucleotide sequence which hybridizes to a nucleic acid selected from the group consisting of SEQ ID NOs.: 8-3795 under stringent conditions, a gene product encoded by a nucleic acid comprising a nucleotide sequence which hybridizes to a nucleic acid selected from the group consisting of SEQ ID NOs.: 8-3795 under moderate conditions, and a gene product whose activity may be complemented by the gene product whose activity is inhibited by a nucleic acid selected from the group consisting of SEQ ID NOs: 8-3795.

35. A method for identifying a gene which is required for proliferation of a cell comprising:
(a) contacting a cell with an antisense nucleic acid selected from the group consisting of a nucleic acid at least 70% nucleotide sequence identity as determined using BLASTN version 2.0 with the default parameters to a nucleotide sequence selected from the group consisting of SEQ ID NOs.: 8-3795 or a proliferation-inhibiting portion thereof, a nucleic acid comprising a nucleotide sequence which hybridizes to a nucleic acid selected from the group consisting of SEQ ID NOs.: 8-3795 under stringent conditions, and a nucleic acid comprising a nucleotide sequence which hybridizes to a nucleic acid selected from the group consisting of SEQ ID NOs.: 8-3795 under moderate conditions, wherein said cell is a cell other than the organism from which said nucleic acid was obtained;
(b) determining whether said nucleic acid inhibits proliferation of said cell; and
(c) identifying the gene in said cell which encodes the mRNA which is complementary to said antisense nucleic acid or a portion thereof.

36. A method for identifying a compound having the ability to inhibit proliferation of a cell comprising:
(a) identifying a homolog of a gene or gene product whose activity or level is inhibited by an antisense nucleic acid in a test cell, wherein said test cell is not the microorgaism from which the antisense nucleic acid was obtained, wherein said antisense nucleic acid is selected from the group consisting of a nucleic acid having at least 70% nucleotide sequence identity as determined using BLASTN version 2.0 with the default parameters to a nucleotide sequence selected from the group consisting of SEQ ID NOs. 8-3795, a nucleic acid comprising a nucleotide sequence which hybridizes to a nucleic acid selected from the group consisting of SEQ ID NOs.: 8-3795 under stringent conditions, and a nucleic acid comprising a nucleotide sequence which hybridizes to a nucleic acid selected from the group consisting of SEQ ID NOs.: 8-3795 under moderate conditions;
(b) identifying an inhibitory nucleic acid sequence which inhibits the activity of said homolog in said test cell;
(c) contacting said test cell with a sublethal level of said inhibitory nucleic acid, thus sensitizing said cell;
(d) contacting the sensitized cell of step (c) with a compound; and
(e) determining the degree to which said compound inhibits proliferation of said sensitized cell relative to a cell which does not express said inhibitory nucleic acid.

37. A method of identifying a compound having the ability to inhibit proliferation comprising:
(a) sensitizing a test cell by contacting said test cell with a sublethal level of an antisense nucleic acid, wherein said antisense nucleic acid is selected from the group consisting of a nucleic acid having at least 70% nucleotide sequence identity as determined using BLASTN version 2.0 with the default parameters to a nucleotide sequence selected from the group consisting of SEQ ID NOs. 8-3795 or a portion thereof which inhibits the proliferation of the cell from which said nucleic acid was obtained, a nucleic acid comprising a nucleotide sequence which hybridizes to a nucleic acid selected from the group consisting of SEQ ID NOs.: 8-3795 under stringent conditions, and a nucleic acid comprising a nucleotide sequence which hybridizes to a nucleic acid selected from the group consisting of SEQ ID NOs.: 8-3795 under moderate conditionst;
(b) contacting the sensitized test cell of step (a) with a compound; and
(c) determining the degree to which said compound inhibits proliferation of said sensitized test cell relative to a cell which does not contain said antisense nucleic acid.

38. A method for identifying a compound having activity against a biological pathway required for proliferation comprising:
(a) sensitizing a cell by providing a sublethal level of an antisense nucleic acid complementary to a nucleic acid encoding a gene product required for proliferation, wherein said gene product is selected from the group consisting of a gene product having at least 70% nucleotide sequence identity as determined using BLASTN version 2.0 with the default parameters to a gene product whose expression is inhibited by an antisense nucleic acid comprising a nucleotide sequence selected from the group consisting of SEQ ID NOs.: 8-3795, a gene product encoded by a nucleic acid having at least 70% nucleotide sequence identity as determined using BLASTN version 2.0 with the default parameters to a nucleic acid encoding a gene product whose expression is inhibited by an antisense nucleic acid comprising a nucleotide sequence selected from the group consisting of SEQ ID NOs:8-3795, a gene product having at least 25% amino acid identity as determined using FASTA version 3.0t78 with the default parameters to a gene product whose expression is inhibited by an antisense nucleic acid comprising a nucleotide sequence selected from the group consisting of SEQ ID NOs.: 8-3795, a gene product encoded by a nucleic acid comprising a nucleotide sequence which hybridizes to a nucleic acid selected from the group consisting of SEQ ID NOs.: 8-3795 under stringent conditions, a gene product encoded by a nucleic acid comprising a nucleotide sequence which hybridizes to a nucleic acid selected from the group consisting of SEQ ID NOs.: 8-3795 under moderate conditions, and a gene product whose activity may be complemented by the gene product whose activity is inhibited by a nucleic acid selected from the group consisting of SEQ ID NOs: 8-3795;
(b) contacting the sensitized cell with a compound; and
(c) determining the extent to which said compound inhibits the growth of said sensitized cell relative to a cell which does not contain said antisense nucleic acid.

39. A method for identifying a compound having the ability to inhibit cellular proliferation comprising:
(a) contacting a cell with an agent which reduces the activity or level of a gene product required for proliferation of said cell, wherein said gene product is selected from the group consisting of a gene product having at least 70% nucleotide sequence identity as determined using BLASTN version 2.0 with the default parameters to a gene product whose expression is inhibited by an antisense nucleic acid comprising a nucleotide sequence selected from the group consisting of SEQ ID NOs.: 8-3795, a gene product encoded by a nucleic acid having at least 70% nucleotide sequence identity as determined using BLASTN version 2.0 with the default parameters to a nucleic acid encoding a gene product whose expression is inhibited by an antisense nucleic acid comprising a nucleotide sequence selected from the group consisting of SEQ ID NOs:8-3795, a gene product having at least 25% amino acid identity as determined using FASTA version 3.0t78 with the default parameters to a gene product whose expression is inhibited by an antisense nucleic acid comprising a nucleotide sequence selected from the group consisting of SEQ ID NOs.: 8-3795, a gene product encoded by a nucleic acid comprising a nucleotide sequence which hybridizes to a nucleic acid selected from the group consisting of SEQ ID NOs.: 8-3795 under stringent conditions, a gene product encoded by a nucleic acid comprising a nucleotide sequence which hybridizes to a nucleic acid selected from the group consisting of SEQ ID NOs.: 8-3795 under moderate conditions, and a gene product whose activity may be complemented by the gene product whose activity is inhibited by a nucleic acid selected from the group consisting of SEQ ID NOs: 8-3795;
(b) contacting said cell with a compound; and
(c) determining the degree to which said compound reduces proliferation of said contacted cell relative to a cell which was not contacted with said agent.

40. A method for identifying the biological pathway in which a proliferation-required gene product or a gene encoding a proliferation-required gene product lies comprising:
(a) providing a sublethal level of an antisense nucleic acid which inhibits the activity or reduces the level of said gene encoding a proliferation-required gene product or said said proliferation-required gene product in a test cell, wherein said proliferation-required gene product is selected from the group consisting of a gene product having at least 70% nucleotide sequence identity as determined using BLASTN version 2.0 with the default parameters to a gene product whose expression is inhibited by an antisense nucleic acid comprising a nucleotide sequence selected from the group consisting of SEQ ID NOs.: 8-3795, a gene product encoded by a nucleic acid having at least 70% nucleotide sequence identity as determined using BLASTN version 2.0 with the default parameters to a nucleic acid encoding a gene product whose expression is inhibited by an antisense nucleic acid comprising a nucleotide sequence selected from the group consisting of SEQ ID NOs:8-3795, a gene product having at least 25% amino acid identity as determined using FASTA version 3.0t78 with the default parameters to a gene product whose expression is inhibited by an antisense nucleic acid comprising a nucleotide sequence selected from the group consisting of SEQ ID NOs.: 8-3795, a gene product encoded by a nucleic acid comprising a nucleotide sequence which hybridizes to a nucleic acid selected from the group consisting of SEQ ID NOs.: 8-3795 under stringent conditions, a gene product encoded by a nucleic acid comprising a nucleotide sequence which hybridizes to a nucleic acid selected from the group consisting of SEQ ID NOs.: 8-3795 under moderate conditions, and a gene product whose activity may be complemented by the gene product whose activity is inhibited by a nucleic acid selected from the group consisting of SEQ ID NOs: 8-3795;
(b) contacting said test cell with a compound known to inhibit growth or proliferation of a cell, wherein the biological pathway on which said compound acts is known; and
(c) determining the degree to which said compound inhibits proliferation of said test cell relative to a cell which does not contain said antisense nucleic acid.

41. A method for determining the biological pathway on which a test compound acts comprising:
(a) providing a sublethal level of an antisense nucleic acid complementary to a proliferation-required nucleic acid in a cell, thereby producing a sensitized cell, wherein said antisense nucleic acid is selected from the group consisting of a nucleic acid having at least 70% nucleotide sequence identity as determined using BLASTN version 2.0 with the default parameters to a nucleotide sequence selected from the group consisting of SEQ ID NOs:8-3795 or a proliferation-inhibiting portion thereof,a nucleic acid comprising a nucleotide sequence which hybridizes to a nucleic acid selected from the group consisting of SEQ ID NOs.: 8-3795 under stringent conditions, and a nucleic acid comprising a nucleotide sequence which hybridizes to a nucleic acid selected from the group consisting of SEQ ID NOs.: 8-3795 under moderate conditions and wherein the biological pathway in which said proliferation-required nucleic acid or a protein encoded by said proliferation-required polypeptide lies is known,
(b) contacting said cell with said test compound; and
(c) determining the degree to which said compound inhibits proliferation of said sensitized cell relative to a cell which does not contain said antisense nucleic acid.

42. A compound which inhibits proliferation by interacting with a gene encoding a gene product required for proliferation or with a gene product required for proliferation, wherein said gene product is selected from the group consisting of a gene product having at least 70% nucleotide sequence identity as determined using BLASTN version 2.0 with the default parameters to a gene product whose expression is inhibited by an anti sense nucleic acid comprising a nucleotide sequence selected from the group consisting of SEQ ID NOs.: 8-3795, a gene product encoded by a nucleic acid having at least 70% nucleotide sequence identity as determined using BLASTN version 2.0 with the default parameters to a nucleic acid encoding a gene product whose expression is inhibited by an antisense nucleic acid comprising a nucleotide sequence selected from the group consisting of SEQ ID NOs:8-3795, a gene product having at least 25% amino acid identity as determined using FASTA version 3.0t78 with the default parameters to a gene product whose expression is inhibited by an antisense nucleic acid comprising a nucleotide sequence selected from the group consisting of SEQ ID NOs.: 8-3795, a gene product encoded by a nucleic acid comprising a nucleotide sequence which hybridizes to a nucleic acid selected from the group consisting of SEQ ID NOs.: 8-3795 under stringent conditions, a gene product encoded by a nucleic acid comprising a nucleotide sequence which hybridizes to a nucleic acid selected from the group consisting of SEQ ID NOs.: 8-3795 under moderate conditions, and a gene product whose activity may be complemented by the gene product whose activity is inhibited by a nucleic acid selected from the group consisting of SEQ ID NOs: 8-3795.

43. A method for manufacturing an antibiotic comprising the steps of:
screening one or more candidate compounds to identify a compound that reduces the activity or level of a gene product required for proliferation wherein said gene product is selected from the group consisting of a gene product having at least 70% nucleotide sequence identity as determined using BLASTN version 2.0 with the default parameters to a gene product whose expression is inhibited by an antisense nucleic acid comprising a nucleotide sequence selected from the group consisting of SEQ ID NOs.: 8-3795, a gene product encoded by a nucleic acid having at least 70% nucleotide sequence identity as determined using BLASTN version 2.0 with the default parameters to a nucleic acid encoding a gene product whose expression is inhibited by an antisense nucleic acid comprising a nucleotide sequence selected from the group consisting of SEQ ID NOs:8-3795, a gene product having at least 25% amino acid identity as determined using FASTA version 3.0t78 with the default parameters to a gene product whose expression is inhibited by an antisense nucleic acid comprising a nucleotide sequence selected from the group consisting of SEQ ID NOs.: 8-3795, a gene product encoded by a nucleic acid comprising a nucleotide sequence which hybridizes to a nucleic acid selected from the group consisting of SEQ ID NOs.: 8-3795 under stringent conditions, a gene product encoded by a nucleic acid comprising a nucleotide sequence which hybridizes to a nucleic acid selected from the group consisting of SEQ ID NOs.: 8-3795 under moderate conditions, and a gene product whose activity may be complemented by the gene product whose activity is inhibited by a nucleic acid selected from the group consisting of SEQ ID NOs: 8-3795; and
manufacturing the compound so identified.

44. A method for inhibiting proliferation of a cell in a subject comprising administering an effective amount of a compound that reduces the activity or level of a gene product required for proliferation of said cell, wherein said gene product is selected from the group consisting of a gene product having at least 70% nucleotide sequence identity as determined using BLASTN version 2.0 with the default parameters to a gene product whose expression is inhibited by an antisense nucleic acid comprising a nucleotide sequence selected from the group consisting of SEQ ID NOs.: 8-3795, a gene product encoded by a nucleic acid having at least 70% nucleotide sequence identity as determined using BLASTN version 2.0 with the default parameters to a nucleic acid encoding a gene product whose expression is inhibited by an antisense nucleic acid comprising a nucleotide sequence selected from the group consisting of SEQ ID NOs:8-3795, a gene product having at least 25% amino acid identity as determined using FASTA version 3.0t78 with the default parameters to a gene product whose expression is inhibited by an antisense nucleic acid comprising a nucleotide sequence selected from the group consisting of SEQ ID NOs.: 8-3795, a gene product encoded by a nucleic acid comprising a nucleotide sequence which hybridizes to a nucleic acid selected from the group consisting of SEQ ID NOs.: 8-3795 under stringent conditions, a gene product encoded by a nucleic acid comprising a nucleotide sequence which hybridizes to a nucleic acid selected from the group consisting of SEQ ID NOs.: 8-3795 under moderate conditions, and a gene product whose activity may be complemented by the gene product whose activity is inhibited by a nucleic acid selected from the group consisting of SEQ ID NOs: 8-3795.
